(19) United States
(12) Patent Application Publication
(10) Pub. No.: US 2026/0308594 A1
(43) Pub. Date: Oct. 8, 2026

(54) TREATMENT OF GPAM RELATED DISEASES AND DISORDERS

(71) Applicant: Empirico Inc., San Diego, CA (US)

(72) Inventors: Omri GOTTESMAN, San Diego, CA (US); Emma BRANDT, Madison, WI (US); Shannon BRUSE, San Diego, CA (US); Brian CAJES, San Diego, CA (US); David JAKUBOSKY, San Diego, CA (US); David LEWIS, Madison, WI (US); Gregory MCINNES, San Diego, CA (US); John VEKICH, San Diego, CA (US); David ROZEMA, Cross Plains, WI (US)

(21) Appl. No.: 18/874,062

(22) PCT Filed: Jun. 20, 2023
(86) PCT No.: PCT/US2023/068741

§ 371 Date: Dec. 11, 2024

Related U.S. Application Data
(60) Provisional application No. 63/354,115, filed on Jun. 21, 2022.
(60) Provisional application No. 63/433,365, filed on Dec. 16, 2022.

(51) Int. Cl. C12N 15/113; A61P 1/16

(52) U.S. Cl. CPC C12N 15/1137; A61P 1/16; C12Y 203/01015; C12N 2310/14; C12N 2310/312; C12N 2310/3125; C12N 2310/313; C12N 2310/314; C12N 2310/315; C12N 2310/316; C12N 2310/321; C12N 2310/322; C12N 2310/3231; C12N 2310/351; C12N 2310/3515

(57) ABSTRACT

Metabolic disorders such as liver disorders and cardiovascular disorders are widely abundant, and may affect a wide variety of people. Improved therapeutics are needed for treating these disorders. Disclosed herein are compositions comprising an oligonucleotide that targets GRAM. The oligonucleotide may include a small interfering RNA (siRNA) or an antisense oligonucleotide (ASO). Also provided herein are methods of treating conditions associated with GRAM gene mutations that include providing an oligonucleotide that targets GRAM in a subject. Some examples of diseases that may be treated include liver diseases or cardiometabolic diseases.

### CROSS-REFERENCE

This application claims the benefit of U.S. Provisional Application No. 63/354,115, filed Jun. 21, 2022, and U.S. Provisional Application No. 63/433,365, filed Dec. 16, 2022, which applications are incorporated herein by reference.

### INCORPORATION BY REFERENCE OF SEQUENCE LISTING

The present application is being filed along with a Sequence Listing in electronic format. The Sequence Listing is provided as a file entitled 54462-742_601_SL.xml, created Jun. 16, 2023, which is 17,347,954 bytes in size. The information in the electronic format of the Sequence Listing is incorporated by reference in its entirety.

### BACKGROUND

Metabolic disorders such as liver disorders and cardiovascular disorders are widely abundant, and may affect a wide variety of people. Improved therapeutics are needed for treating these disorders.

### SUMMARY

Described herein are compositions that target GPAM. Described herein are compositions comprising an oligonucleotide that targets GPAM and when administered to a subject in an effective amount reduces a GPAM mRNA or protein level. A composition comprising an oligonucleotide that targets GPAM and when administered to a subject in an effective amount decreases circulating cholesterol, apolipoprotein B, bilirubin, alanine aminotransferase, aspartate aminotransferase, or aspartate aminotransferase in a subject. In some embodiments, the cholesterol comprises total cholesterol, low density lipoprotein cholesterol, or non-high density lipoprotein cholesterol. In some embodiments, the decreased by about 10% or more, as compared to prior to administration. Described herein are compositions comprising an oligonucleotide that targets GPAM and when administered to a subject in an effective amount decreases a liver fibrosis score, non-alcoholic fatty liver disease (NAFLD) activity score, or liver fat percentage in a subject. In some embodiments, the decrease is by about 10% or more, as compared to prior to administration. Described herein are compositions comprising an oligonucleotide that targets GPAM and when administered to a subject in an effective amount decreases a use of statin (HMG CoA reductase inhibitor) medication. In some embodiments, the decrease is by about 10% or more, as compared to prior to administration. Described herein are compositions comprising an oligonucleotide that targets GPAM and when administered to a subject in an effective amount decreases a measurement that reflects a phenotype of esophageal varices, portal hypertension, NAFLD, NASH, alcoholic liver disease, liver fibrosis, liver cirrhosis, hepatocellular carcinoma, hyperlipidemia, ischemic heart disease, or coronary heart disease in a subject. In some embodiments, the decrease is by about 10% or more, as compared to prior to administration. Described herein are compositions comprising an oligonucleotide that targets GPAM and when administered to a subject in an effective amount increases circulating ketone bodies in a subject. In some embodiments, the increase is by about 10% or more, as compared to prior to administration. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-O-(2-methoxyethyl), 2′-O-alkyl, 2′-O-allyl, 2′-O-allyl, 2′-fluoro, or 2′-deoxy, or a combination thereof. In some embodiments, the modified nucleoside comprises an LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the oligonucleotide comprises 1, 2, 3, 4, 5, 6, 7, 8, 9, 10, 11, 12, 13, 14, 15, 16, 17, 18, 19, 20, or 21 modified nucleosides. In some embodiments, the oligonucleotide comprises a hydrophobic moiety attached at a 3′ or 5′ terminus of the oligonucleotide. In some embodiments, the hydrophobic moiety comprises cholesterol, myristoyl, palmitoyl, stearoyl, lithocholoyl, docosanoyl, docosahexaenoyl, myristyl, palmityl, stearyl, or α-tocopherol, or a combination thereof. In some embodiments, the hydrophobic moiety comprises stearyl, t-butylphenyl, n-butylphenyl, octylphenyl, dodecylphenyl, phenyl n-dodecyl, octadecylbenzamide, hexadecylbenzamide, or octadecylcyclohexyl. In some embodiments, the hydrophobic moiety comprises any one of the following structures:

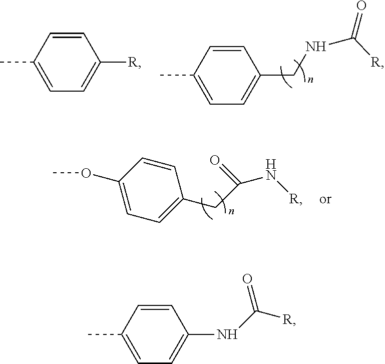

wherein the dotted line indicates a covalent connection to an end of the oligonucleotide, n is 1-3, and R is an alkyl group containing 4-18 carbons. In some embodiments, the oligonucleotide comprises an N-acetylgalactosamine (GalNAc) moiety, an N-acetylglucosamine (GlcNAc) moiety, or a mannose moiety, attached at a 3′ or 5′ terminus of the oligonucleotide. In some embodiments, the oligonucleotide comprises a GalNAc moiety. Some embodiments include:

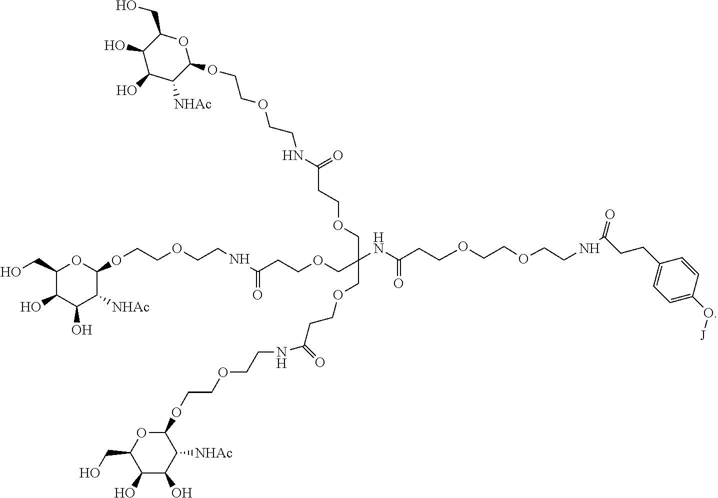

wherein J comprises the oligonucleotide, and wherein J comprises an optional phosphate or phosphorothioate linking to the oligonucleotide. In some embodiments, the oligonucleotide comprises a small interfering RNA (siRNA) comprising a sense strand and an antisense strand. In some embodiments, the sense strand is 12-30 nucleosides in length. In some embodiments, the antisense strand is 12-30 nucleosides in length. Described herein are compositions comprising an oligonucleotide that inhibits the expression of GPAM, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 12867. In some embodiments, any one of the following is true with regard to the sense strand: (i) all purines comprise 2′ fluoro modified purines, and all pyrimidines comprise a mixture of 2′ fluoro and 2′-O-methyl modified pyrimidines; (ii) all purines comprise 2′-O-methyl modified purines, and all pyrimidines comprise a mixture of 2′ fluoro and 2′-O-methyl modified pyrimidines; (iii) all purines comprise 2′ fluoro modified purines, and all pyrimidines comprise 2′-O-methyl modified pyrimidines; (iv) all pyrimidines comprise 2′ fluoro modified pyrimidines, and all purines comprise a mixture of 2′ fluoro and 2′-O-methyl modified purines; (v) all pyrimidines comprise 2′-O-methyl modified pyrimidines, and all purines comprise a mixture of 2′ fluoro and 2′-O-methyl modified purines; or (vi) all pyrimidines comprise 2′ fluoro modified pyrimidines, and all purines comprise 2′-O-methyl modified purines. In some embodiments, the sense strand comprises any one of modification patterns 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 545, 55S, 56S, 57S, 58S, 59S, 60S, 61S, 62S, 63S, 64S, 65S, 66S, 67S, 68S, 69S, or 70S. In some embodiments, any one of the following is true with regard to the antisense strand: (i) all purines comprise 2′ fluoro modified purines, and all pyrimidines comprise a mixture of 2′ fluoro and 2′-O-methyl modified pyrimidines; (ii) all purines comprise 2′-O-methyl modified purines, and all pyrimidines comprise a mixture of 2′ fluoro and 2′-O-methyl modified pyrimidines; (iii) all purines comprise 2′-O-methyl modified purines, and all pyrimidines comprise 2′ fluoro modified pyrimidines; (iv) all pyrimidines comprise 2′ fluoro modified pyrimidines, and all purines comprise a mixture of 2′ fluoro and 2′-O-methyl modified purines; (v) all pyrimidines comprise 2′-O-methyl modified pyrimidines, and all purines comprise a mixture of 2′ fluoro and 2′-O-methyl modified purines; or (vi) all pyrimidines comprise 2′-O-methyl modified pyrimidines, and all purines comprise 2′ fluoro modified purines. In some embodiments, the antisense strand comprises any one of modification patterns 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the oligonucleotide comprises an antisense oligonucleotide (ASO). In some embodiments, the ASO is 12-30 nucleosides in length. Described herein are compositions comprising an oligonucleotide that inhibits the expression of GPAM, wherein the oligonucleotide comprises an ASO about 12-30 nucleosides in length and a nucleoside sequence complementary to about 12-30 contiguous nucleosides of SEQ ID NO: 12867. Some embodiments include a pharmaceutically acceptable carrier. Described herein are methods of treating a subject having liver disease, comprising administering an effective amount of the composition of any one of claims 1-42 to the subject. In some embodiments, the liver disease comprises NAFLD, NASH, alcoholic liver disease, liver fibrosis, liver cirrhosis, or hepatocellular carcinoma. Described herein are methods of treating a subject having cardiometabolic disease, comprising administering an effective amount of the composition of any one of claims 1-42 to the subject. In some embodiments, the cardiometabolic disease comprises hyperlipidemia, ischemic heart disease, or coronary heart disease.

### DETAILED DESCRIPTION

Large-scale human genetic data can improve the success rate of pharmaceutical discovery and development. A Genome Wide Association Study (GWAS) detects associations between genetic variants and traits in a population sample, and this improves understanding of the biology of disease and provides evidence of applicable treatments. A GWAS generally utilizes genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is considered associated with disease. Association statistics used in a GWAS include p-values, as a measure of statistical significance; odds ratios (OR), as a measure of effect size, or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation is used to identify a causal genetic variant identified via GWAS, and in many cases leads to identification of disease-causing genes. In particular, understanding the functional effect of a causal genetic variant (for example, loss of protein function, gain of protein function, increase in gene expression, or decrease in gene expression) allows that variant to be used as a proxy for therapeutic modulation of the target gene, or to gain insight into potential therapeutic efficacy and safety of a therapeutic that modulates that target.

Identification of such gene-disease associations has provided insights into disease biology and is used to identify novel therapeutic targets for the pharmaceutical industry. In order to translate the therapeutic insights derived from human genetics, disease biology in patients is exogenously ‘programmed’ into replicating the observation from human genetics. There are several options for therapeutic modalities that may be brought to bear in translating therapeutic targets identified via human genetics into novel medicines. These include well established therapeutic modalities such as small molecules and monoclonal antibodies, maturing modalities such as oligonucleotides, and emerging modalities such as gene therapy and gene editing. The choice of therapeutic modality depends on factors such as the location of a target (for example, intracellular, extracellular, or secreted), a relevant tissue (for example, fat or liver) and a relevant indication.

The GPAM (also known as GPAT or GPAT1) gene is located on chromosome 10, and encodes glycerol-3-phosphate acyltransferase, mitochondrial (GPAM). GPAM may include 828 amino acids and have a mass of about 94 kDa. GPAM may be expressed in liver, adipose, adrenal, thyroid, heart, gall bladder, brain, salivary gland, and testis cells. GPAM may be intracellular. GPAM may exist as two different enzymatic forms, in the mitochondria or in the endoplasmic reticulum. GPAM may catalyze the initial and committing step in glycerolipid biosynthesis and can play a significant role in the regulation of cellular triacylglycerol and phospholipid levels. The mitochondrial enzyme GPAM may preferentially use saturated fatty acids as a substrate for the synthesis of glycerolipids. GPAM may catalyze the first step in this metabolic pathway. GPAM may interact with APP, SREBF1, AGPAT1, AGPAT2, AGPAT3, AGPAT4, AGPAT5, AGPAT6, AGPAT9, GPD1, or MBOAT2. An example of an 828 amino acid sequence, and further description of GPAM is included at uniprot.org under accession no. Q9HCL2 (last modified May 25, 2022). A potential alternatively spliced isoform of GPAM producing a 710 amino acid sequence is included at uniprot.org under accession no. Q5VW52 (last modified Dec. 7, 2004).

Here it is shown that genetic loss-of-function GPAM variants result in protective blood ketone associations, liver disease-related associations, liver function associations, and blood lipid associations. Therefore, inhibition of GPAM may serve as a therapeutic for treatment of liver or cardiometabolic diseases or disorders such as non-alcoholic fatty liver disease (NAFLD), non-alcoholic steatohepatitis (NASH), alcoholic liver disease, liver fibrosis, liver cirrhosis, hepatocellular carcinoma, hyperlipidemia, ischemic heart disease, or coronary heart disease. The GPAM inhibition may result in an improved liver function, cardiovascular function, and metabolic phenotypes including favorable liver fat percentage, liver fibrosis score, NAFLD activity score, liver enzyme function test, serum metabolite test, or serum lipid panel test.

Disclosed herein are compositions comprising an oligonucleotide that targets GPAM. Where inhibition or targeting of GPAM is disclosed, it is contemplated that some embodiments may include inhibiting or targeting a GPAM protein or GPAM RNA. For example, by inhibiting or targeting an RNA (e.g. mRNA) encoded by the GPAM gene using an oligonucleotide described herein, the GPAM protein may be inhibited or targeted as a result of there being less production of the GPAM protein by translation of the GPAM RNA; or a GPAM protein may be targeted or inhibited by an oligonucleotide that binds or interacts with a GPAM RNA and reduces production of the GPAM protein from the GPAM RNA. Thus, targeting GPAM may refer to binding a GPAM RNA and reducing GPAM RNA or protein levels. The oligonucleotide may include a small interfering RNA (siRNA) or an antisense oligonucleotide (ASO). Administration of the oligonucleotide to a subject may improve (e.g. decrease) a liver fat percentage, liver fibrosis score, NAFLD activity score, blood alanine aminotransferase (ALT), blood aspartate aminotransferase (AST), blood aspartate aminotransferase (ASP), blood bilirubin, low-density lipoprotein (LDL), total cholesterol, non-HDL cholesterol, or apolipoprotein B (APOB) measurement, or a combination thereof in the subject. Also provided herein are methods of treating a liver or cardiometabolic disorder by providing an oligonucleotide that targets GPAM to a subject in need thereof

### I. COMPOSITIONS

Disclosed herein, in some embodiments, are compositions comprising an oligonucleotide. In some embodiments, the composition comprises an oligonucleotide that targets GPAM. In some embodiments, the composition consists of an oligonucleotide that targets GPAM. In some embodiments, the oligonucleotide reduces GPAM mRNA expression in the subject. In some embodiments, the oligonucleotide reduces GPAM protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder as described herein.

Some embodiments include a composition comprising an oligonucleotide that targets GPAM and when administered to a subject in an effective amount decreases GPAM mRNA or protein levels in a cell, fluid or tissue. In some embodiments, the composition comprises an oligonucleotide that targets GPAM and when administered to a subject in an effective amount decreases GPAM mRNA levels in a cell or tissue. In some embodiments, the tissue is liver tissue. In some embodiments, the tissue is fat tissue. In some embodiments, the cell is a hepatocyte. In some embodiments, the cell is an adipocyte. In some embodiments, the GPAM mRNA levels are decreased by about 2.5% or more, about 5% or more, or about 7.5% or more, as compared to prior to administration. In some embodiments, the GPAM mRNA levels are decreased by about 10% or more, as compared to prior to administration. In some embodiments, the GPAM mRNA levels are decreased by about 20% or more, about 30% or more, about 40% or more, about 50% or more, about 60% or more, about 70% or more, about 80% or more, about 90% or more, or about 100%, as compared to prior to administration. In some embodiments, the GPAM mRNA levels are decreased by no more than about 2.5%, no more than about 5%, or no more than about 7.5%, as compared to prior to administration. In some embodiments, the GPAM mRNA levels are decreased by no more than about 10%, as compared to prior to administration. In some embodiments, the GPAM mRNA levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the GPAM mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets GPAM and when administered to a subject in an effective amount decreases GPAM protein levels in a cell, fluid or tissue. In some embodiments, the composition decreases GPAM protein levels in a cell or tissue. In some embodiments, the tissue is liver tissue. In some embodiments, the tissue is fat tissue. In some embodiments, the cell is a hepatocyte. In some embodiments, the cell is an adipocyte. In some embodiments, the GPAM protein levels are decreased by about 2.5% or more, about 5% or more, or about 7.5% or more, as compared to prior to administration. In some embodiments, the GPAM protein levels are decreased by about 10% or more, as compared to prior to administration. In some embodiments, the GPAM protein levels are decreased by about 20% or more, about 30% or more, about 40% or more, about 50% or more, about 60% or more, about 70% or more, about 80% or more, about 90% or more, or about 100%, as compared to prior to administration. In some embodiments, the GPAM protein levels are decreased by no more than about 2.5%, no more than about 5%, or no more than about 7.5%, as compared to prior to administration. In some embodiments, the GPAM protein levels are decreased by no more than about 10%, as compared to prior to administration. In some embodiments, the GPAM protein levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the GPAM protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets GPAM and when administered to a subject in an effective amount diminishes a liver disease phenotype. The liver disease may include non-alcoholic fatty liver disease (NAFLD), non-alcoholic steatohepatitis (NASH), alcoholic liver disease, liver fibrosis, liver cirrhosis, or hepatocellular carcinoma. In some embodiments, the liver disease phenotype is decreased by about 2.5% or more, about 5% or more, or about 7.5% or more, as compared to prior to administration. In some embodiments, the liver disease phenotype is decreased by about 10% or more, as compared to prior to administration. In some embodiments, the liver disease phenotype is decreased by about 20% or more, about 30% or more, about 40% or more, about 50% or more, about 60% or more, about 70% or more, about 80% or more, about 90% or more, or about 100%, as compared to prior to administration. In some embodiments, the liver disease phenotype is decreased by no more than about 2.5%, no more than about 5%, or no more than about 7.5%, as compared to prior to administration. In some embodiments, the liver disease phenotype is decreased by no more than about 10%. as compared to prior to administration. In some embodiments, the liver disease phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the liver disease phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets GPAM and when administered to a subject in an effective amount diminishes a cardiometabolic disease phenotype. The cardiometabolic disease may include hyperlipidemia, ischemic heart disease, or coronary heart disease. In some embodiments, the cardiometabolic disease phenotype is decreased by about 2.5% or more, about 5% or more, or about 7.5% or more, as compared to prior to administration. In some embodiments, the cardiometabolic disease phenotype is decreased by about 10% or more, as compared to prior to administration. In some embodiments, the cardiometabolic disease phenotype is decreased by about 20% or more, about 30% or more, about 40% or more, about 50% or more, about 60% or more, about 70% or more, about 80% or more, about 90% or more, or about 100%, as compared to prior to administration. In some embodiments, the cardiometabolic disease phenotype is decreased by no more than about 2.5%, no more than about 5%, or no more than about 7.5%, as compared to prior to administration. In some embodiments, the cardiometabolic disease phenotype is decreased by no more than about 10%, as compared to prior to administration. In some embodiments, the cardiometabolic disease phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the cardiometabolic disease phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets GPAM and when administered to a subject in an effective amount enhances a protective phenotype against a liver disease in the subject. The liver disease may include non-alcoholic fatty liver disease (NAFLD), non-alcoholic steatohepatitis (NASH), alcoholic liver disease, liver fibrosis, liver cirrhosis, or hepatocellular carcinoma. In some embodiments, the protective phenotype is increased by about 2.5% or more, about 5% or more, or about 7.5% or more, as compared to prior to administration. In some embodiments, the protective phenotype is increased by about 10% or more, as compared to prior to administration. In some embodiments, the protective phenotype is increased by about 20% or more, about 30% or more, about 40% or more, about 50% or more, about 60% or more, about 70% or more, about 80% or more, about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more, about 900% or more, or about 1000% or more, as compared to prior to administration. In some embodiments, the protective phenotype is increased by no more than about 2.5%, no more than about 50%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets GPAM and when administered to a subject in an effective amount enhances a protective phenotype against a cardiometabolic disease in the subject. The cardiometabolic disease may include hyperlipidemia, ischemic heart disease, or coronary heart disease. In some embodiments, the protective phenotype is increased by about 2.5% or more, about 5% or more, or about 7.5% or more, as compared to prior to administration. In some embodiments, the protective phenotype is increased by about 10% or more, as compared to prior to administration. In some embodiments, the protective phenotype is increased by about 20% or more, about 30% or more, about 40% or more, about 50% or more, about 60% or more, about 70% or more, about 80% or more, about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more, about 900% or more, or about 1000% or more, as compared to prior to administration. In some embodiments, the protective phenotype is increased by no more than about 2.5%, no more than about 5%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150% 200%, 250% 300% 400%, 500% 600%, 700%, 800%, 900%, or 1000%, or by a range defined by any of the two aforementioned percentages.

In some embodiments, the composition comprises an oligonucleotide that targets GPAM and when administered to a subject in an effective amount decreases circulating cholesterol in the subject. The circulating cholesterol may include total cholesterol or non-high density lipoprotein (HDL) cholesterol. The circulating cholesterol may include total cholesterol. The circulating cholesterol may include non-HDL cholesterol. In some embodiments, the circulating cholesterol is decreased by about 2.5% or more, about 5% or more, or about 7.5% or more, as compared to prior to administration. In some embodiments, the circulating cholesterol is decreased by about 10% or more, as compared to prior to administration. In some embodiments, the circulating cholesterol is decreased by about 20% or more, about 30% or more, about 40% or more, about 50% or more, about 60% or more, about 70% or more, about 80% or more, or about 90% or more, or about 100%, as compared to prior to administration. In some embodiments, the circulating cholesterol is decreased by no more than about 2.5%, no more than about 5%, or no more than about 7.5%, as compared to prior to administration. In some embodiments, the circulating cholesterol is decreased by no more than about 10%, as compared to prior to administration. In some embodiments, the circulating cholesterol is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating cholesterol is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases circulating low density lipoproteins (LDL) in the subject. In some embodiments, the circulating LDL is decreased by about 2.5% or more, about 50% or more, or about 7.5% or more, as compared to prior to administration. In some embodiments, the circulating LDL is decreased by about 10% or more, as compared to prior to administration. In some embodiments, the circulating LDL is decreased by about 20% or more, about 30% or more, about 40% or more, about 50% or more, about 60% or more, about 70% or more, about 80% or more, or about 90% or more, or about 100%, as compared to prior to administration. In some embodiments, the circulating LDL is decreased by no more than about 2.5%, no more than about 5%, or no more than about 7.5%, as compared to prior to administration. In some embodiments, the circulating LDL is decreased by no more than about 10%, as compared to prior to administration. In some embodiments, the circulating LDL is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating LDL is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases circulating apolipoprotein B (APOB) in the subject. In some embodiments, the circulating APOB is decreased by about 2.5% or more, about 5% or more, or about 7.5% or more, as compared to prior to administration. In some embodiments, the circulating APOB is decreased by about 10% or more, as compared to prior to administration. In some embodiments, the circulating APOB is decreased by about 20% or more, about 30% or more, about 40% or more, about 50% or more, about 60% or more, about 70% or more, about 80% or more, or about 90% or more, or about 100%, as compared to prior to administration. In some embodiments, the circulating APOB is decreased by no more than about 2.5%, no more than about 5%, or no more than about 7.5%, as compared to prior to administration. In some embodiments, the circulating APOB is decreased by no more than about 10%, as compared to prior to administration. In some embodiments, the circulating APOB is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating APOB is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases circulating alanine aminotransferase (ALT) in the subject. In some embodiments, the circulating ALT is decreased by about 2.5% or more, about 50% or more, or about 7.5% or more, as compared to prior to administration. In some embodiments, the circulating ALT is decreased by about 10% or more, as compared to prior to administration. In some embodiments, the circulating ALT is decreased by about 20% or more, about 30% or more, about 40% or more, about 50% or more, about 60% or more, about 70% or more, about 80% or more, or about 90% or more, or about 100%, as compared to prior to administration. In some embodiments, the circulating ALT is decreased by no more than about 2.5%, no more than about 5%, or no more than about 7.5%, as compared to prior to administration. In some embodiments, the circulating ALT is decreased by no more than about 10%, as compared to prior to administration. In some embodiments, the circulating ALT is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating ALT is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases circulating aspartate aminotransferase (AST) in the subject. In some embodiments, the circulating AST is decreased by about 2.5% or more, about 5% or more, or about 7.5% or more, as compared to prior to administration. In some embodiments, the circulating AST is decreased by about 10% or more, as compared to prior to administration. In some embodiments, the circulating AST is decreased by about 20% or more, about 30% or more, about 40% or more, about 50% or more, about 60% or more, about 70% or more, about 80% or more, or about 90% or more, or about 100%, as compared to prior to administration. In some embodiments, the circulating AST is decreased by no more than about 2.5%, no more than about 5%, or no more than about 7.5%, as compared to prior to administration. In some embodiments, the circulating AST is decreased by no more than about 10%, as compared to prior to administration. In some embodiments, the circulating AST is decreased by no more than about 20%, no more than about 30%, no more than about 40%, no more than about 50%, no more than about 60%, no more than about 70%. no more than about 80%, or no more than about 90%, as compared to prior to administration. In some embodiments, the circulating AST is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases circulating aspartate aminotransferase (ASP) in the subject. In some embodiments, the circulating ASP is decreased by about 2.5% or more, about 5% or more, or about 7.5% or more, as compared to prior to administration. In some embodiments, the circulating ASP is decreased by about 10% or more, as compared to prior to administration. In some embodiments, the circulating ASP is decreased by about 20% or more, about 30% or more, about 40% or more, about 50% or more, about 60% or more, about 70% or more, about 80% or more, or about 90% or more, or about 100%, as compared to prior to administration. In some embodiments, the circulating ASP is decreased by no more than about 2.5%, no more than about 5%, or no more than about 7.5%, as compared to prior to administration. In some embodiments, the circulating ASP is decreased by no more than about 10%, as compared to prior to administration. In some embodiments, the circulating ASP is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating ASP is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases circulating bilirubin in the subject. In some embodiments, the circulating bilirubin is decreased by about 20.5% or more, about 5% or more, or about 7.5% or more, as compared to prior to administration. In some embodiments, the circulating bilirubin is decreased by about 10% or more, as compared to prior to administration. In some embodiments, the circulating bilirubin is decreased by about 20% or more, about 30% or more, about 40% or more, about 50% or more, about 60% or more, about 70% or more, about 80% or more, or about 90% or more, or about 100%, as compared to prior to administration. In some embodiments, the circulating bilirubin is decreased by no more than about 2.5%, no more than about 5%, or no more than about 7.5%, as compared to prior to administration. In some embodiments, the circulating bilirubin is decreased by no more than about 10%, as compared to prior to administration. In some embodiments, the circulating bilirubin is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating bilirubin is decreased by 2.5%, 5%, 7.5%, 10%, 15%, 20%, %, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases a nonalcoholic fatty liver disease (NAFLD) activity score in the subject. In some embodiments, the NAFLD activity score is decreased by about 2.5% or more, about 5% or more, or about 7.5% or more, as compared to prior to administration. In some embodiments, the NAFLD activity score is decreased by about 10% or more, as compared to prior to administration. In some embodiments, the NAFLD activity score is decreased by about 20% or more, about 30% or more, about 40% or more, about 50% or more, about 60% or more, about 70% or more, about 80% or more, or about 90% or more, or about 100%, as compared to prior to administration. In some embodiments, the NAFLD activity score is decreased by no more than about 2.5%, no more than about 50%, or no more than about 7.5%, as compared to prior to administration. In some embodiments, the NAFLD activity score is decreased by no more than about 10%, as compared to prior to administration. In some embodiments, the NAFLD activity score is decreased by no more than about 20%, no more than about 30%, no more than about 40%, no more than about 50%, no more than about 60%, no more than about 70%, no more than about 80%, or no more than about 90%, as compared to prior to administration. In some embodiments, the NAFLD activity score is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases a liver fat percentage in the subject. In some embodiments, the liver fat percentage is decreased by about 2.5% or more, about 5% or more, or about 7.5% or more, as compared to prior to administration. In some embodiments, the liver fat percentage is decreased by about 10% or more, as compared to prior to administration. In some embodiments, the liver fat percentage is decreased by about 20% or more, about 30% or more, about 40% or more, about 50% or more, about 60% or more, about 70% or more, about 80% or more, or about 90% or more, or about 100%, as compared to prior to administration. In some embodiments, the liver fat percentage is decreased by no more than about 2.5%, no more than about 5%, or no more than about 7.5%, as compared to prior to administration. In some embodiments, the liver fat percentage is decreased by no more than about 10%, as compared to prior to administration. In some embodiments, the liver fat percentage is decreased by no more than about 20%, no more than about 30%, no more than about 40%, no more than about 50%, no more than about 60%, no more than about 70%, no more than about 80%, or no more than about 90%, as compared to prior to administration. In some embodiments, the liver fat percentage is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases a liver fibrosis score in the subject. In some embodiments, the liver fibrosis score is decreased by about 2.5% or more, about 5% or more, or about 7.5% or more, as compared to prior to administration. In some embodiments, the liver fibrosis score is decreased by about 10% or more, as compared to prior to administration. In some embodiments, the liver fibrosis score is decreased by about 20% or more, about 30% or more, about 40% or more, about 50% or more, about 60% or more, about 70% or more, about 80% or more, or about 90% or more, or about 100%, as compared to prior to administration. In some embodiments, the liver fibrosis score is decreased by no more than about 2.5%, no more than about 5%, or no more than about 7.5%, as compared to prior to administration. In some embodiments, the liver fibrosis score is decreased by no more than about 10%, as compared to prior to administration. In some embodiments, the liver fibrosis score is decreased by no more than about 20%, no more than about 30%, no more than about 40%, no more than about 50%, no more than about 60%, no more than about 70%, no more than about 80%, or no more than about 90%, as compared to prior to administration. In some embodiments, the liver fibrosis score is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount decreases a use of an HGM CoA reductase inhibitor (e.g. a statin) medication in the subject. In some embodiments, the use of statin medications is decreased by about 2.5% or more, about 5% or more, or about 7.5% or more, as compared to prior to administration. In some embodiments, the use of statin medications is decreased by about 10% or more, as compared to prior to administration. In some embodiments, the use of statin medications is decreased by about 20% or more, about 30% or more, about 40% or more, about 50% or more, about 60% or more, about 70% or more, about 80% or more, or about 90% or more, or about 100%, as compared to prior to administration. In some embodiments, the use of statin medications is decreased by no more than about 2.5%, no more than about 5%, or no more than about 7.5%, as compared to prior to administration. In some embodiments, the use of statin medications is decreased by no more than about 10%, as compared to prior to administration. In some embodiments, the use of statin medications is decreased by no more than about 20%, no more than about 30%, no more than about 40%, no more than about 50%, no more than about 60%, no more than about 70%, no more than about 80%, or no more than about 90%, as compared to prior to administration. In some embodiments, the use of statin medications is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 100%, or by a range defined by any of the two aforementioned percentages.

In some embodiments, the composition comprises an oligonucleotide that targets GPAM and when administered to a subject in an effective amount increases circulating ketone bodies in the subject. In some embodiments, the circulating ketone body 3-hydroxybutyrate is increased by about 2.5% or more, about 50% or more, or about 7.50% or more, as compared to prior to administration. In some embodiments, the circulating ketone body 3-hydroxybutyrate is increased by about 10% or more, as compared to prior to administration. In some embodiments, the circulating ketone body 3-hydroxybutyrate is increased by about 20% or more, about 30% or more, about 40% or more, about 50% or more, about 60% or more, about 70% or more, about 80% or more, about 90% or more, or about 100% or more, as compared to prior to administration. In some embodiments, the circulating ketone body 3-hydroxybutyrate is increased by about 200% or more, about 300% or more, about 400% or more, about 500% or more, about 600% or more, about 700% or more, about 800% or more, about 900% or more, or about 1000% or more, as compared to prior to administration. In some embodiments, the circulating ketone body 3-hydroxybutyrate is increased by no more than about 2.5%, no more than about 5%, or no more than about 7.5%, as compared to prior to administration. In some embodiments, the circulating ketone body 3-hydroxybutyrate is increased by no more than about 10%, as compared to prior to administration. In some embodiments, the circulating ketone body 3-hydroxybutyrate is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the circulating ketone body 3-hydroxybutyrate is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the circulating ketone body 3-hydroxybutyrate is increased by 2.5%, 5%, 7.5%, 10%, 15%, 20%, 25%, 30%, 35%, 40%, 45%, 50%, 55%, 60%/, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets GPAM and when administered to a subject in an effective amount increases circulating ketone bodies in the subject. In some embodiments, the circulating ketone body acetoacetate is increased by about 2.5% or more, about 5% or more, or about 7.5% or more, as compared to prior to administration. In some embodiments, the circulating ketone body acetoacetate is increased by about 10% or more, as compared to prior to administration. In some embodiments, the circulating ketone body acetoacetate is increased by about 20% or more, about 30% or more, about 40% or more, about 50% or more, about 60% or more, about 70% or more, about 80% or more, about 90% or more, or about 100% or more, as compared to prior to administration. In some embodiments, the circulating ketone body acetoacetate is increased by about 200% or more, about 300% or more, about 400% or more, about 500% or more, about 600% or more, about 700% or more, about 800% or more, about 900% or more, or about 1000% or more, as compared to prior to administration. In some embodiments, the circulating ketone body acetoacetate is increased by no more than about 2.5%, no more than about 5%, or no more than about 7.5%, as compared to prior to administration. In some embodiments, the circulating ketone body acetoacetate is increased by no more than about 10%, as compared to prior to administration. In some embodiments, the circulating ketone body acetoacetate is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the circulating ketone body acetoacetate is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the circulating ketone body acetoacetate is increased by 2.5%, 5%, 7.5%, 10%, 15%, 20%, 25%, 30%, 35%, 40%, 45%, 50%, 55%, 60%, 65%, 70%, 75%, 80%, 85%0, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets GPAM and when administered to a subject in an effective amount increases circulating ketone bodies in the subject. In some embodiments, the circulating ketone body acetone is increased by about 2.5% or more, about 5% or more, or about 7.5% or more, as compared to prior to administration. In some embodiments, the circulating ketone body acetone is increased by about 10% or more, as compared to prior to administration. In some embodiments, the circulating ketone body acetone is increased by about 20% or more, about 30% or more, about 40% or more, about 50% or more, about 60% or more, about 70% or more, about 80% or more, about 90% or more, or about 100% or more, as compared to prior to administration. In some embodiments, the circulating ketone body acetone is increased by about 200% or more, about 300% or more, about 400% or more, about 500% or more, about 600% or more, about 700% or more, about 800% or more, about 900% or more, or about 1000% or more, as compared to prior to administration. In some embodiments, the circulating ketone body acetone is increased by no more than about 2.5%, no more than about 5%, or no more than about 7.5%, as compared to prior to administration. In some embodiments, the circulating ketone body acetone is increased by no more than about 10%, as compared to prior to administration. In some embodiments, the circulating ketone body acetone is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the circulating ketone body acetone is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the circulating ketone body acetone is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

A. siRNAs

In some embodiments, the composition comprises an oligonucleotide that targets GPAM, wherein the oligonucleotide comprises a small interfering RNA (siRNA). In some embodiments, the composition comprises an oligonucleotide that targets GPAM, wherein the oligonucleotide comprises a small interfering RNA (siRNA) comprising a sense strand and an antisense strand.

In some embodiments, the composition comprises an oligonucleotide that inhibits the expression of GPAM, wherein the oligonucleotide comprises an siRNA comprising a sense strand and an antisense strand, wherein the sense strand is 12-30 nucleosides in length. In some embodiments, the composition comprises a sense strange that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The sense strand may be 14-30 nucleosides in length. In some embodiments, the composition comprises an antisense strand is 12-30 nucleosides in length. In some embodiments, the composition comprises an antisense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The antisense strand may be 14-30 nucleosides in length.

In some embodiments, the composition comprises an oligonucleotide that inhibits the expression of GPAM, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of a full-length human GPAM mRNA sequence such as SEQ ID NO: 12867. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 12867.

In some embodiments, the composition comprises an oligonucleotide that inhibits the expression of GPAM, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of GPAM, wherein the oligonucleotide comprises an siRNA comprising a sense strand and an antisense strand, wherein the siRNA binds with a 19mer in a human GPAM mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human GPAM mRNA.

In some embodiments, the composition comprises an oligonucleotide that inhibits the expression of GPAM, wherein the oligonucleotide comprises an siRNA comprising a sense strand and an antisense strand, wherein the siRNA binds with a 17mer in a non-human primate GPAM mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate GPAM mRNA.

In some embodiments, the composition comprises an oligonucleotide that inhibits the expression of GPAM, wherein the oligonucleotide comprises an siRNA comprising a sense strand and an antisense strand, wherein the siRNA binds with a human GPAM mRNA and less than or equal to 20 human off-targets, with no more than 2 mismatches in the antisense strand. In some embodiments, the siRNA binds with a human GPAM mRNA and less than or equal to 10 human off-targets, with no more than 2 mismatches in the antisense strand. In some embodiments, the siRNA binds with a human GPAM mRNA and less than or equal to 30 human off-targets, with no more than 2 mismatches in the antisense strand. In some embodiments, the siRNA binds with a human GPAM mRNA and less than or equal to 40 human off-targets, with no more than 2 mismatches in the antisense strand. In some embodiments, the siRNA binds with a human GPAM mRNA and less than or equal to 50 human off-targets, with no more than 2 mismatches in the antisense strand. In some embodiments, the siRNA binds with a human GPAM mRNA and less than or equal to 10 human off-targets, with no more than 3 mismatches in the antisense strand. In some embodiments, the siRNA binds with a human GPAM mRNA and less than or equal to 20 human off-targets, with no more than 3 mismatches in the antisense strand. In some embodiments, the siRNA binds with a human GPAM mRNA and less than or equal to 30 human off-targets, with no more than 3 mismatches in the antisense strand. In some embodiments, the siRNA binds with a human GPAM mRNA and less than or equal to 40 human off-targets, with no more than 3 mismatches in the antisense strand. In some embodiments, the siRNA binds with a human GPAM mRNA and less than or equal to 50 human off-targets, with no more than 3 mismatches in the antisense strand.

In some embodiments, the composition comprises an oligonucleotide that inhibits the expression of GPAM, wherein the oligonucleotide comprises an siRNA comprising a sense strand and an antisense strand, siRNA binds with a human GPAM mRNA target site that does not harbor an SNP, with a minor allele frequency (MAF) greater or equal to 1% (pos. 2-18). In some embodiments, the MAF is greater or equal to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the siRNA comprises a sense strand having a sequence in accordance with any of SEQ ID NOs: 1-6354. In some embodiments, the sense strand sequence comprises or consists of sequence at least 75% identical to any one of SEQ ID NOs: 1-6354, at least 80% identical to any one of SEQ ID NOs: 1-6354, at least 85% identical to of any one of SEQ ID NOs: 1-6354, at least 90% identical to any one of SEQ ID NOs 1-6354, or at least 95% identical to any one of SEQ ID NOs: 1-6354. In some embodiments, the sense strand sequence comprises or consists of the sequence of any one of SEQ ID NOs: 1-6354, or a sense strand sequence thereof having 1, 2, 3, or 4 nucleoside substitutions, additions, or deletions. In some embodiments, the sense strand sequence comprises or consists of the sequence of any one of SEQ ID NOs: 1-6354, or a sense strand sequence thereof having 1 or 2 nucleoside substitutions, additions, or deletions. In some embodiments, the sense strand sequence comprises or consists of a sequence 100% identical to SEQ ID NOs: 1-6354. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise a modification pattern described herein. The sense strand may comprise an overhang. The sense strand may comprise a lipid moiety. The sense strand may comprise a GalNAc moiety. In some embodiments, the siRNA comprises an antisense strand having a sequence in accordance with any of SEQ ID NOs: 6355-12708. In some embodiments, the antisense strand sequence comprises or consists of sequence at least 75% identical to any one of SEQ ID NOs: 6355-12708, at least 80% identical to any one of SEQ ID NOs: 6355-12708, at least 85% identical to of any one of SEQ ID NOs: 6355-12708, at least 90% identical to any one of SEQ ID NOs: 6355-12708, or at least 95% identical to any one of SEQ ID NOs: 6355-12708. In some embodiments, the antisense strand sequence comprises or consists of the sequence of any one of SEQ ID NOs: 6355-12708, or an antisense strand sequence thereof having 1, 2, 3, or 4 nucleoside substitutions, additions, or deletions. In some embodiments, the antisense strand sequence comprises or consists of the sequence of any one of SEQ ID NOs: 6355-12708, or an antisense strand sequence thereof having 1 or 2 nucleoside substitutions, additions, or deletions. In some embodiments, the antisense strand sequence comprises or consists of a sequence 100% identical to SEQ ID NOs: 6355-12708. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein. The antisense strand may comprise a lipid moiety or a GalNAc moiety.

In some embodiments, the siRNA comprises a sense strand having a sequence in accordance with any of SEQ ID NOs: 13082-13402. In some embodiments, the sense strand sequence comprises or consists of sequence at least 75% identical to any one of SEQ ID NOs: 13082-13402, at least 80% identical to any one of SEQ ID NOs: 13082-13402, at least 85% identical to of any one of SEQ ID NOs: 13082-13402, at least 90% identical to any one of SEQ ID NOs 13082-13402, or at least 95% identical to any one of SEQ ID NOs: 13082-13402. In some embodiments, the sense strand sequence comprises or consists of the sequence of any one of SEQ ID NOs: 13082-13402, or a sense strand sequence thereof having 1, 2, 3, or 4 nucleoside substitutions, additions, or deletions. In some embodiments, the sense strand sequence comprises or consists of the sequence of any one of SEQ ID NOs: 13082-13402, or a sense strand sequence thereof having 1 or 2 nucleoside substitutions, additions, or deletions. In some embodiments, the sense strand sequence comprises or consists of a sequence 100% identical to SEQ ID NOs: 13082-13402. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise a modification pattern described herein. The sense strand may comprise an overhang. The sense strand may comprise a lipid moiety. The sense strand may comprise a GalNAc moiety. In some embodiments, the siRNA comprises an antisense strand having a sequence in accordance with any of SEQ ID NOs: 13403-13723. In some embodiments, the antisense strand sequence comprises or consists of sequence at least 75% identical to any one of SEQ ID NOs: 13403-13723, at least 80% identical to any one of SEQ ID NOs: 13403-13723, at least 85% identical to of any one of SEQ ID NOs: 13403-13723, at least 90% identical to any one of SEQ ID NOs: 13403-13723, or at least 95% identical to any one of SEQ ID NOs: 13403-13723. In some embodiments, the antisense strand sequence comprises or consists of the sequence of any one of SEQ ID NOs: 13403-13723, or an antisense strand sequence thereof having 1, 2, 3, or 4 nucleoside substitutions, additions, or deletions. In some embodiments, the antisense strand sequence comprises or consists of the sequence of any one of SEQ ID NOs: 13403-13723, or an antisense strand sequence thereof having 1 or 2 nucleoside substitutions, additions, or deletions. In some embodiments, the antisense strand sequence comprises or consists of a sequence 100% identical to SEQ ID NOs: 13403-13723. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein. The antisense strand may comprise a lipid moiety or a GalNAc moiety.

In some embodiments, the siRNA comprises a sense strand or antisense strand having a sequence in accordance with the sense strand or antisense strand sequence of an siRNA of subset A. In some embodiments, the sense strand or antisense strand comprises a sequence at least 75% identical, at least 80% identical, at least 85% identical, at least 90% identical, or at least 95% identical, to a sense strand or antisense strand sequence of subset A. In some embodiments, the sense strand or antisense strand comprises a sequence of a sense or antisense strand of subset A, or a sequence thereof having 3 or 4 nucleoside substitutions, additions, or deletions. In some embodiments, the sense strand or antisense strand comprises a sequence of a sense or antisense strand of subset A, or a sequence thereof having 1 or 2 nucleoside substitutions, additions, or deletions. In some embodiments, the sense strand or antisense sequence comprises or consists of a sequence 100% identical to a sense strand or antisense strand sequence of subset A. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset B. In some embodiments, the sense strand or antisense strand comprises a sequence at least 75% identical, at least 80% identical, at least 85% identical, at least 90% identical, or at least 95% identical, to a sense strand or antisense strand sequence of subset B. In some embodiments, the sense strand or antisense strand comprises a sequence of a sense or antisense strand of subset B, or a sequence thereof having 3 or 4 nucleoside substitutions, additions, or deletions. In some embodiments, the sense strand or antisense strand comprises a sequence of a sense or antisense strand of subset B, or a sequence thereof having 1 or 2 nucleoside substitutions, additions, or deletions. In some embodiments, the sense strand or antisense sequence comprises or consists of a sequence 100% identical to a sense strand or antisense strand sequence of subset B. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset C. In some embodiments, the sense strand or antisense strand comprises a sequence at least 750% identical, at least 80% identical, at least 85% identical, at least 90% identical, or at least 95% identical, to a sense strand or antisense strand sequence of subset C. In some embodiments, the sense strand or antisense strand comprises a sequence of a sense or antisense strand of subset C, or a sequence thereof having 3 or 4 nucleoside substitutions, additions, or deletions. In some embodiments, the sense strand or antisense strand comprises a sequence of a sense or antisense strand of subset C, or a sequence thereof having 1 or 2 nucleoside substitutions, additions, or deletions. In some embodiments, the sense strand or antisense sequence comprises or consists of a sequence 100% identical to a sense strand or antisense strand sequence of subset C. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset D. In some embodiments, the sense strand or antisense strand comprises a sequence at least 750% identical, at least 80% identical, at least 85% identical, at least 90% identical, or at least 95% identical, to a sense strand or antisense strand sequence of subset D. In some embodiments, the sense strand or antisense strand comprises a sequence of a sense or antisense strand of subset D, or a sequence thereof having 3 or 4 nucleoside substitutions, additions, or deletions. In some embodiments, the sense strand or antisense strand comprises a sequence of a sense or antisense strand of subset D, or a sequence thereof having 1 or 2 nucleoside substitutions, additions, or deletions. In some embodiments, the sense strand or antisense sequence comprises or consists of a sequence 100% identical to a sense strand or antisense strand sequence of subset D. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset E. In some embodiments, the sense strand or antisense strand comprises a sequence at least 75% identical, at least 80% identical, at least 85% identical, at least 90% identical, or at least 95% identical, to a sense strand or antisense strand sequence of subset E. In some embodiments, the sense strand or antisense strand comprises a sequence of a sense or antisense strand of subset E, or a sequence thereof having 3 or 4 nucleoside substitutions, additions, or deletions. In some embodiments, the sense strand or antisense strand comprises a sequence of a sense or antisense strand of subset E, or a sequence thereof having 1 or 2 nucleoside substitutions, additions, or deletions. In some embodiments, the sense strand or antisense sequence comprises or consists of a sequence 100% identical to a sense strand or antisense strand sequence of subset E. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset F. In some embodiments, the sense strand or antisense strand comprises a sequence at least 750% identical, at least 80% identical, at least 85% identical, at least 90% identical, or at least 95% identical, to a sense strand or antisense strand sequence of subset F. In some embodiments, the sense strand or antisense strand comprises a sequence of a sense or antisense strand of subset F, or a sequence thereof having 3 or 4 nucleoside substitutions, additions, or deletions. In some embodiments, the sense strand or antisense strand comprises a sequence of a sense or antisense strand of subset F, or a sequence thereof having 1 or 2 nucleoside substitutions, additions, or deletions. In some embodiments, the sense strand or antisense sequence comprises or consists of a sequence 100% identical to a sense strand or antisense strand sequence of subset F. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset I. In some embodiments, the sense strand or antisense strand comprises a sequence at least 750% identical, at least 80% identical, at least 85% identical, at least 90% identical, or at least 95% identical, to a sense strand or antisense strand sequence of subset I. In some embodiments, the sense strand or antisense strand comprises a sequence of a sense or antisense strand of subset I, or a sequence thereof having 3 or 4 nucleoside substitutions, additions, or deletions. In some embodiments, the sense strand or antisense strand comprises a sequence of a sense or antisense strand of subset I, or a sequence thereof having 1 or 2 nucleoside substitutions, additions, or deletions. In some embodiments, the sense strand or antisense sequence comprises or consists of a sequence 100% identical to a sense strand or antisense strand sequence of subset I. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset J. In some embodiments, the sense strand or antisense strand comprises a sequence at least 750% identical, at least 80% identical, at least 85% identical, at least 90% identical, or at least 95% identical, to a sense strand or antisense strand sequence of subset J. In some embodiments, the sense strand or antisense strand comprises a sequence of a sense or antisense strand of subset J, or a sequence thereof having 3 or 4 nucleoside substitutions, additions, or deletions. In some embodiments, the sense strand or antisense strand comprises a sequence of a sense or antisense strand of subset J, or a sequence thereof having 1 or 2 nucleoside substitutions, additions, or deletions. In some embodiments, the sense strand or antisense sequence comprises or consists of a sequence 100% identical to a sense strand or antisense strand sequence of subset J. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA herein (such as an siRNA in a table herein). In some embodiments, the sense strand or antisense strand comprises a sequence at least 75% identical, at least 80% identical, at least 85% identical, at least 90% identical, or at least 95% identical, to a sense strand or antisense strand sequence of an siRNA herein. In some embodiments, the sense strand or antisense strand comprises a sequence of a sense or antisense strand of an siRNA herein, or a sequence thereof having 3 or 4 nucleoside substitutions, additions, or deletions. In some embodiments, the sense strand or antisense strand comprises a sequence of a sense or antisense strand of an siRNA herein, or a sequence thereof having 1 or 2 nucleoside substitutions, additions, or deletions. In some embodiments, the sense strand or antisense sequence comprises or consists of a sequence 100% identical to a sense strand or antisense strand sequence of an siRNA herein. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 8. In some embodiments, the sense strand or antisense strand comprises a sequence at least 75% identical, at least 80% identical, at least 85% identical, at least 90% identical, or at least 95% identical, to a sense strand or antisense strand sequence of Table 8. In some embodiments, the sense strand or antisense strand comprises a sequence of a sense or antisense strand of Table 8, or a sequence thereof having 3 or 4 nucleoside substitutions, additions, or deletions. In some embodiments, the sense strand or antisense strand comprises a sequence of a sense or antisense strand of Table 8, or a sequence thereof having 1 or 2 nucleoside substitutions, additions, or deletions. In some embodiments, the sense strand or antisense sequence comprises or consists of a sequence 100% identical to a sense strand or antisense strand sequence of Table 8. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 9. In some embodiments, the sense strand or antisense strand comprises a sequence at least 75% identical, at least 80% identical, at least 85% identical, at least 90% identical, or at least 95% identical, to a sense strand or antisense strand sequence of Table 9. In some embodiments, the sense strand or antisense strand comprises a sequence of a sense or antisense strand of Table 9, or a sequence thereof having 3 or 4 nucleoside substitutions, additions, or deletions. In some embodiments, the sense strand or antisense strand comprises a sequence of a sense or antisense strand of Table 9, or a sequence thereof having 1 or 2 nucleoside substitutions, additions, or deletions. In some embodiments, the sense strand or antisense sequence comprises or consists of a sequence 100% identical to a sense strand or antisense strand sequence of Table 9. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 11A. In some embodiments, the sense strand or antisense strand comprises a sequence at least 750% identical, at least 80% identical, at least 85% identical, at least 90% identical, or at least 95% identical, to a sense strand or antisense strand sequence of Table 11A. In some embodiments, the sense strand or antisense strand comprises a sequence of a sense or antisense strand of Table 11A, or a sequence thereof having 3 or 4 nucleoside substitutions, additions, or deletions. In some embodiments, the sense strand or antisense strand comprises a sequence of a sense or antisense strand of Table 11A, or a sequence thereof having 1 or 2 nucleoside substitutions, additions, or deletions. In some embodiments, the sense strand or antisense sequence comprises or consists of a sequence 100% identical to a sense strand or antisense strand sequence of Table 11A. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 11B. In some embodiments, the sense strand or antisense strand comprises a sequence at least 750% identical, at least 80% identical, at least 85% identical, at least 90% identical, or at least 95% identical, to a sense strand or antisense strand sequence of Table 11B. In some embodiments, the sense strand or antisense strand comprises a sequence of a sense or antisense strand of Table 11B, or a sequence thereof having 3 or 4 nucleoside substitutions, additions, or deletions. In some embodiments, the sense strand or antisense strand comprises a sequence of a sense or antisense strand of Table 11B, or a sequence thereof having 1 or 2 nucleoside substitutions, additions, or deletions. In some embodiments, the sense strand or antisense sequence comprises or consists of a sequence 100% identical to a sense strand or antisense strand sequence of Table 11B. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 14. In some embodiments, the sense strand or antisense strand comprises a sequence at least 75% identical, at least 80% identical, at least 85% identical, at least 90% identical, or at least 95% identical, to a sense strand or antisense strand sequence of Table 14. In some embodiments, the sense strand or antisense strand comprises a sequence of a sense or antisense strand of Table 14, or a sequence thereof having 3 or 4 nucleoside substitutions, additions, or deletions. In some embodiments, the sense strand or antisense strand comprises a sequence of a sense or antisense strand of Table 14, or a sequence thereof having 1 or 2 nucleoside substitutions, additions, or deletions. In some embodiments, the sense strand or antisense sequence comprises or consists of a sequence 100% identical to a sense strand or antisense strand sequence of Table 14. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 18. In some embodiments, the sense strand or antisense strand comprises a sequence at least 75% identical, at least 80% identical, at least 85% identical, at least 90% identical, or at least 95% identical, to a sense strand or antisense strand sequence of Table 18. In some embodiments, the sense strand or antisense strand comprises a sequence of a sense or antisense strand of Table 18, or a sequence thereof having 3 or 4 nucleoside substitutions, additions, or deletions. In some embodiments, the sense strand or antisense strand comprises a sequence of a sense or antisense strand of Table 18, or a sequence thereof having 1 or 2 nucleoside substitutions, additions, or deletions. In some embodiments, the sense strand or antisense sequence comprises or consists of a sequence 100% identical to a sense strand or antisense strand sequence of Table 18. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 22. In some embodiments, the sense strand or antisense strand comprises a sequence at least 75% identical, at least 80% identical, at least 85% identical, at least 90% identical, or at least 95% identical, to a sense strand or antisense strand sequence of Table 22. In some embodiments, the sense strand or antisense strand comprises a sequence of a sense or antisense strand of Table 22, or a sequence thereof having 3 or 4 nucleoside substitutions, additions, or deletions. In some embodiments, the sense strand or antisense strand comprises a sequence of a sense or antisense strand of Table 22, or a sequence thereof having 1 or 2 nucleoside substitutions, additions, or deletions. In some embodiments, the sense strand or antisense sequence comprises or consists of a sequence 100% identical to a sense strand or antisense strand sequence of Table 22. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 25. In some embodiments, the sense strand or antisense strand comprises a sequence at least 750% identical, at least 80% identical, at least 85% identical, at least 90% identical, or at least 95% identical, to a sense strand or antisense strand sequence of Table 25. In some embodiments, the sense strand or antisense strand comprises a sequence of a sense or antisense strand of Table 25, or a sequence thereof having 3 or 4 nucleoside substitutions, additions, or deletions. In some embodiments, the sense strand or antisense strand comprises a sequence of a sense or antisense strand of Table 25, or a sequence thereof having 1 or 2 nucleoside substitutions, additions, or deletions. In some embodiments, the sense strand or antisense sequence comprises or consists of a sequence 100% identical to a sense strand or antisense strand sequence of Table 25. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 28. In some embodiments, the sense strand or antisense strand comprises a sequence at least 750% identical, at least 80% identical, at least 85% identical, at least 90% identical, or at least 95% identical, to a sense strand or antisense strand sequence of Table 28. In some embodiments, the sense strand or antisense strand comprises a sequence of a sense or antisense strand of Table 28, or a sequence thereof having 3 or 4 nucleoside substitutions, additions, or deletions. In some embodiments, the sense strand or antisense strand comprises a sequence of a sense or antisense strand of Table 28, or a sequence thereof having 1 or 2 nucleoside substitutions, additions, or deletions. In some embodiments, the sense strand or antisense sequence comprises or consists of a sequence 100% identical to a sense strand or antisense strand sequence of Table 28. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 31. In some embodiments, the sense strand or antisense strand comprises a sequence at least 750% identical, at least 80% identical, at least 85% identical, at least 90% identical, or at least 95% identical, to a sense strand or antisense strand sequence of Table 31. In some embodiments, the sense strand or antisense strand comprises a sequence of a sense or antisense strand of Table 31, or a sequence thereof having 3 or 4 nucleoside substitutions, additions, or deletions. In some embodiments, the sense strand or antisense strand comprises a sequence of a sense or antisense strand of Table 31, or a sequence thereof having 1 or 2 nucleoside substitutions, additions, or deletions. In some embodiments, the sense strand or antisense sequence comprises or consists of a sequence 100% identical to a sense strand or antisense strand sequence of Table 31. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 35. In some embodiments, the sense strand or antisense strand comprises a sequence at least 750% identical, at least 80% identical, at least 85% identical, at least 90% identical, or at least 95% identical, to a sense strand or antisense strand sequence of Table 35. In some embodiments, the sense strand or antisense strand comprises a sequence of a sense or antisense strand of Table 35, or a sequence thereof having 3 or 4 nucleoside substitutions, additions, or deletions. In some embodiments, the sense strand or antisense strand comprises a sequence of a sense or antisense strand of Table 35, or a sequence thereof having 1 or 2 nucleoside substitutions, additions, or deletions. In some embodiments, the sense strand or antisense sequence comprises or consists of a sequence 100% identical to a sense strand or antisense strand sequence of Table 35. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 49. In some embodiments, the sense strand or antisense strand comprises a sequence at least 750% identical, at least 80% identical, at least 85% identical, at least 90% identical, or at least 95% identical, to a sense strand or antisense strand sequence of Table 49. In some embodiments, the sense strand or antisense strand comprises a sequence of a sense or antisense strand of Table 49, or a sequence thereof having 3 or 4 nucleoside substitutions, additions, or deletions. In some embodiments, the sense strand or antisense strand comprises a sequence of a sense or antisense strand of Table 49, or a sequence thereof having 1 or 2 nucleoside substitutions, additions, or deletions. In some embodiments, the sense strand or antisense sequence comprises or consists of a sequence 100% identical to a sense strand or antisense strand sequence of Table 49. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 63. In some embodiments, the sense strand or antisense strand comprises a sequence at least 75% identical, at least 80% identical, at least 85% identical, at least 90% identical, or at least 95% identical, to a sense strand or antisense strand sequence of Table 63. In some embodiments, the sense strand or antisense strand comprises a sequence of a sense or antisense strand of Table 63, or a sequence thereof having 3 or 4 nucleoside substitutions, additions, or deletions. In some embodiments, the sense strand or antisense strand comprises a sequence of a sense or antisense strand of Table 63, or a sequence thereof having 1 or 2 nucleoside substitutions, additions, or deletions. In some embodiments, the sense strand or antisense sequence comprises or consists of a sequence 100% identical to a sense strand or antisense strand sequence of Table 63. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 66. In some embodiments, the sense strand or antisense strand comprises a sequence at least 750% identical, at least 80% identical, at least 85% identical, at least 90% identical, or at least 95% identical, to a sense strand or antisense strand sequence of Table 66. In some embodiments, the sense strand or antisense strand comprises a sequence of a sense or antisense strand of Table 66, or a sequence thereof having 3 or 4 nucleoside substitutions, additions, or deletions. In some embodiments, the sense strand or antisense strand comprises a sequence of a sense or antisense strand of Table 66, or a sequence thereof having 1 or 2 nucleoside substitutions, additions, or deletions. In some embodiments, the sense strand or antisense sequence comprises or consists of a sequence 100% identical to a sense strand or antisense strand sequence of Table 66. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand having a sequence in accordance with any of SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274. In some embodiments, the sense strand sequence comprises or consists of sequence at least 75% identical to any one of SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274., at least 80% identical to any one of SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274., at least 85% identical to of any one of SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274., at least 90% identical to any one of SEQ ID NOs 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274., or at least 95% identical to any one of SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274.. In some embodiments, the sense strand sequence comprises or consists of the sequence of any one of SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274., or a sense strand sequence thereof having 1, 2, 3, or 4 nucleoside substitutions, additions, or deletions. In some embodiments, the sense strand sequence comprises or consists of the sequence of any one of SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274., or a sense strand sequence thereof having 1 or 2 nucleoside substitutions, additions, or deletions. In some embodiments, the sense strand sequence comprises or consists of a sequence 100% identical to SEQ ID NOs: 13329, 13334, 13335, 13091, 13259, 13260, 13261, 13262, 13263, 13264, 13265, 13266, 13336, 13311, 13312, 13341, 13099, 13165, 13166, 13167, 13168, 13169, 13170, 13171, 13172, 13173, 13100, 13157, 13158, 13159, 13160, 13161, 13162, 13163, 13164, 13344, 13103, 13216, 13217, 13218, 13219, 13220, 13221, 13222, 13223, 13224, 13307, 13308, 13309, 13310, 13345, 13346, 13106, 13267, 13268, 13269, 13270, 13271, 13272, 13273, and 13274.. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise a modification pattern described herein. The sense strand may comprise an overhang. The sense strand may comprise a lipid moiety. The sense strand may comprise a GalNAc moiety.

In some embodiments, the siRNA comprises an antisense strand having a sequence in accordance with any of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595. In some embodiments, the antisense strand sequence comprises or consists of sequence at least 75% identical to any one of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595, at least 80% identical to any one of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595, at least 85% identical to of any one of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595, at least 90% identical to any one of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595, or at least 95% identical to any one of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595. In some embodiments, the antisense strand sequence comprises or consists of the sequence of any one of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595, or an antisense strand sequence thereof having 1, 2, 3, or 4 nucleoside substitutions, additions, or deletions. In some embodiments, the antisense strand sequence comprises or consists of the sequence of any one of SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595, or an antisense strand sequence thereof having 1 or 2 nucleoside substitutions, additions, or deletions. In some embodiments, the antisense strand sequence comprises or consists of a sequence 100% identical to SEQ ID NOs: 13650, 13655, 13656, 13412, 13580, 13581, 13582, 13583, 13584, 13585, 13586, 13587, 13657, 13632, 13633, 13662, 13420, 13486, 13487, 13488, 13489, 13490, 13491, 13492, 13493, 13494, 13421, 13478, 13479, 13480, 13481, 13482, 13483, 13484, 13485, 13665, 13424, 13537, 13538, 13539, 13540, 13541, 13542, 13543, 13544, 13545, 13628, 13629, 13630, 13631, 13666, 13667, 13427, 13588, 13589, 13590, 13591, 13592, 13593, 13594, or 13595. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein. The antisense strand may comprise a lipid moiety or a GalNAc moiety.

In some embodiments, the siRNA comprises a sense strand having a sequence in accordance with any of SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300. In some embodiments, the sense strand sequence comprises or consists of sequence at least 75% identical to any one of SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300, at least 80% identical to any one of SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300, at least 85% identical to of any one of SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300, at least 90% identical to any one of SEQ ID NOs 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300, or at least 95% identical to any one of SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300. In some embodiments, the sense strand sequence comprises or consists of the sequence of any one of SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300, or a sense strand sequence thereof having 1, 2, 3, or 4 nucleoside substitutions, additions, or deletions. In some embodiments, the sense strand sequence comprises or consists of the sequence of any one of SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300, or a sense strand sequence thereof having 1 or 2 nucleoside substitutions, additions, or deletions. In some embodiments, the sense strand sequence comprises or consists of a sequence 100% identical to SEQ ID NOs: 13329, 13334, 13335, 13260, 13264, 13336, 13311, 13341, 13168, 13173, 13161, 13344, 13224, 13345, 13346, 13267, 13268, 13353, 13358, 13359, 13276, 13280, 13360, 13317, 13365, 13185, 13190, 13178, 13368, 13241, 13369, 13370, 13283, 13284, 13377, 13382, 13383, 13292, 13296, 13384, 13323, 13389, 13202, 13207, 13195, 13392, 13258, 13393, 13394, 13299, or 13300. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise a modification pattern described herein. The sense strand may comprise an overhang. The sense strand may comprise a lipid moiety. The sense strand may comprise a GalNAc moiety.

In some embodiments, the siRNA comprises an antisense strand having a sequence in accordance with any of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621. In some embodiments, the antisense strand sequence comprises or consists of sequence at least 75% identical to any one of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621, at least 80% identical to any one of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621, at least 85% identical to of any one of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621, at least 90% identical to any one of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621, or at least 95% identical to any one of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621. In some embodiments, the antisense strand sequence comprises or consists of the sequence of any one of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621, or an antisense strand sequence thereof having 1, 2, 3, or 4 nucleoside substitutions, additions, or deletions. In some embodiments, the antisense strand sequence comprises or consists of the sequence of any one of SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621, or an antisense strand sequence thereof having 1 or 2 nucleoside substitutions, additions, or deletions. In some embodiments, the antisense strand sequence comprises or consists of a sequence 100% identical to SEQ ID NOs: 13650, 13655, 13656, 13581, 13585, 13657, 13632, 13662, 13489, 13494, 13482, 13665, 13545, 13666, 13667, 13588, 13589, 13674, 13679, 13680, 13597, 13601, 13681, 13638, 13686, 13506, 13511, 13499, 13689, 13562, 13690, 13691, 13604, 13605, 13698, 13703, 13704, 13613, 13617, 13705, 13644, 13710, 13523, 13528, 13516, 13713, 13579, 13714, 13715, 13620, or 13621. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein. The antisense strand may comprise a lipid moiety or a GalNAc moiety.

### B. ASOs

In some embodiments, the composition comprises an oligonucleotide that inhibits the expression of GPAM, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

In some embodiments, the composition comprises an oligonucleotide that inhibits the expression of GPAM, wherein the oligonucleotide comprises an ASO about 12-30 nucleosides in length and comprising a nucleoside sequence complementary to about 12-30 contiguous nucleosides of a full-length human GPAM mRNA sequence such as SEQ ID NO: 12867; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 12867.

### C. Oligonucleotide Modifications

In some embodiments, the composition comprises an oligonucleotide that inhibits the expression of GPAM, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. A phosphorothioate may include a nonbridging oxygen atom in a phosphate backbone of the oligonucleotide that is replaced by sulfur. Modified internucleoside linkages may be included in siRNAs or ASOs. Benefits of the modified internucleoside linkage may include decreased toxicity or improved pharmacokinetics.

In some embodiments, the composition comprises an oligonucleotide that inhibits the expression of GPAM, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of GPAM, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-O-(2-methoxyethyl), 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-(2-methoxyethyl) group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-(2-methoxyethyl). In some embodiments, the modified nucleoside comprises a 2-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

In some embodiments, the composition comprises an oligonucleotide that inhibits the expression of GPAM, wherein the oligonucleotide comprises a moiety attached at a 3′ or 5′ terminus of the oligonucleotide. Examples of moieties include a hydrophobic moiety or a sugar moiety, or a combination thereof. In some embodiments, the oligonucleotide is an siRNA having a sense strand, and the moiety is attached to a 5′ end of the sense strand. In some embodiments, the oligonucleotide is an siRNA having a sense strand, and the moiety is attached to a 3′ end of the sense strand. In some embodiments, the oligonucleotide is an siRNA having an antisense strand, and the moiety is attached to a 5′ end of the antisense strand. In some embodiments, the oligonucleotide is an siRNA having an antisense strand, and the moiety is attached to a 3′ end of the antisense strand. In some embodiments, the oligonucleotide is an ASO, and the moiety is attached to a 5′ end of the ASO. In some embodiments, the oligonucleotide is an ASO, and the moiety is attached to a 3′ end of the ASO.

The oligonucleotide may include purines. Examples of purines include adenine (A) or guanine (G), or modified versions thereof. The oligonucleotide may include pyrimidines. Examples of pyrimidines include cytosine (C), thymine (T), or uracil (U), or modified versions thereof.

In some embodiments, purines of the oligonucleotide comprise 2′ fluoro modified purines. In some embodiments, purines of the oligonucleotide comprise 2′-O-methyl modified purines. In some embodiments, purines of the oligonucleotide comprise a mixture of 2′ fluoro and 2′-O-methyl modified purines. In some embodiments, all purines of the oligonucleotide comprise 2′ fluoro modified purines. In some embodiments, all purines of the oligonucleotide comprise 2′-O-methyl modified purines. In some embodiments, all purines of the oligonucleotide comprise a mixture of 2′ fluoro and 2′-O-methyl modified purines. 2′-O-methyl may include 2′ O-methyl.

In some embodiments, pyrimidines of the oligonucleotide comprise 2′ fluoro modified pyrimidines. In some embodiments, pyrimidines of the oligonucleotide comprise 2′-O-methyl modified pyrimidines. In some embodiments, pyrimidines of the oligonucleotide comprise a mixture of 2′ fluoro and 2′-O-methyl modified pyrimidines. In some embodiments, all pyrimidines of the oligonucleotide comprise 2′ fluoro modified pyrimidines. In some embodiments, all pyrimidines of the oligonucleotide comprise 2′-O-methyl modified pyrimidines. In some embodiments, all pyrimidines of the oligonucleotide comprise a mixture of 2′ fluoro and 2′-O-methyl modified pyrimidines.

In some embodiments, purines of the oligonucleotide comprise 2′ fluoro modified purines, and pyrimidines of the oligonucleotide comprise a mixture of 2′ fluoro and 2′-O-methyl modified pyrimidines. In some embodiments, purines of the oligonucleotide comprise 2′-O-methyl modified purines, and pyrimidines of the oligonucleotide comprise a mixture of 2′ fluoro and 2′-O-methyl modified pyrimidines. In some embodiments, purines of the oligonucleotide comprise 2′ fluoro modified purines, and pyrimidines of the oligonucleotide comprise 2′-O-methyl modified pyrimidines. In some embodiments, purines of the oligonucleotide comprise 2′-O-methyl modified purines, and pyrimidines of the oligonucleotide comprise 2′ fluoro modified pyrimidines. In some embodiments, pyrimidines of the oligonucleotide comprise 2′ fluoro modified pyrimidines, and purines of the oligonucleotide comprise a mixture of 2′ fluoro and 2′-O-methyl modified purines. In some embodiments, pyrimidines of the oligonucleotide comprise 2′-O-methyl modified pyrimidines, and purines of the oligonucleotide comprise a mixture of 2′ fluoro and 2′-O-methyl modified purines. In some embodiments, pyrimidines of the oligonucleotide comprise 2′ fluoro modified pyrimidines, and purines of the oligonucleotide comprise 2′-O-methyl modified purines. In some embodiments, pyrimidines of the oligonucleotide comprise 2′-O-methyl modified pyrimidines, and purines of the oligonucleotide comprise 2′ fluoro modified purines.

In some embodiments, all purines of the oligonucleotide comprise 2′ fluoro modified purines, and all pyrimidines of the oligonucleotide comprise a mixture of 2′ fluoro and 2′-O-methyl modified pyrimidines. In some embodiments, all purines of the oligonucleotide comprise 2′-O-methyl modified purines, and all pyrimidines of the oligonucleotide comprise a mixture of 2′ fluoro and 2′-O-methyl modified pyrimidines. In some embodiments, all purines of the oligonucleotide comprise 2′ fluoro modified purines, and all pyrimidines of the oligonucleotide comprise 2′-O-methyl modified pyrimidines. In some embodiments, all purines of the oligonucleotide comprise 2′-O-methyl modified purines, and all pyrimidines of the oligonucleotide comprise 2′ fluoro modified pyrimidines. In some embodiments, all pyrimidines of the oligonucleotide comprise 2′ fluoro modified pyrimidines, and all purines of the oligonucleotide comprise a mixture of 2′ fluoro and 2′-O-methyl modified purines. In some embodiments, all pyrimidines of the oligonucleotide comprise 2′-O-methyl modified pyrimidines, and all purines of the oligonucleotide comprise a mixture of 2′ fluoro and 2′-O-methyl modified purines. In some embodiments, all pyrimidines of the oligonucleotide comprise 2′ fluoro modified pyrimidines, and all purines of the oligonucleotide comprise 2′-O-methyl modified purines. In some embodiments, all pyrimidines of the oligonucleotide comprise 2′-O-methyl modified pyrimidines, and all purines of the oligonucleotide comprise 2′ fluoro modified purines.

In some cases, the oligonucleotide comprises a particular modification pattern. In some embodiments, position 9 counting from the 5′ end of the of a strand of the oligonucleotide may have a 2′F modification. In some embodiments, when position 9 of a strand of the oligonucleotide is a pyrimidine, then all purines in a strand of the oligonucleotide have a 2′OMe modification. In some embodiments, when position 9 is the only pyrimidine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are pyrimidines, then both of these pyrimidines are the only two positions with a 2′F modification in a strand of the oligonucleotide. In some embodiments, when position 9 and only two other bases between positions 5 and 11 of a strand of the oligonucleotide are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of a strand of the oligonucleotide, then all combinations of pyrimidines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some embodiments, when position 9 of a strand of the oligonucleotide is a purine, then all purines in a strand of the oligonucleotide have a 2′OMe modification. In some embodiments, when position 9 is the only purine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are purines, then both of these purines are the only two positions with a 2′F modification in a strand of the oligonucleotide. In some embodiments, when position 9 and only two other bases between positions 5 and 11 of a strand of the oligonucleotide are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of a strand of the oligonucleotide, then all combinations of purines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some cases, position 9 of a strand of the oligonucleotide can be a 2′deoxy. In these cases, 2′F and 2′OMe modifications may occur at the other positions of a strand of the oligonucleotide. In some cases, a strand of the oligonucleotide of any of the siRNAs comprises a modification pattern which conforms to these a strand of the oligonucleotide rules.

In some embodiments, position nine of the sense strand comprises a 2′ fluoro-modified pyrimidine. In some embodiments, all purines of the sense strand comprise 2′-O-methyl modified purines. In some embodiments, 1, 2, 3, 4, or 5 pyrimidines between positions 5 and 11 comprise a 2′flouro-modified pyrimidine, provided there are not three 2′ fluoro-modified pyrimidines in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified pyrimidine; all purines of the sense strand comprises 2′-O-methyl modified purines; 1, 2, 3, 4, or 5 pyrimidines between positions 5 and 11 comprise a 2′flouro-modified pyrimidine, provided there are not three 2′ fluoro-modified pyrimidines in a row; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises a 2′ fluoro-modified purine. In some embodiments, all pyrimidines of the sense strand comprise 2′-O-methyl modified purines. In some embodiments, 1, 2, 3, 4, or 5 purines between positions 5 and 11 comprise a 2′flouro-modified purine, provided there are not three 2′ fluoro-modified purine in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified purine; all pyrimidine of the sense strand comprises 2′-O-methyl modified pyrimidines; 1, 2, 3, 4, or 5 purines between positions 5 and 11 comprise a 2′flouro-modified purines, provided there are not three 2′ fluoro-modified purines in a row; the odd-numbered positions of the antisense strand comprise 2′-O-methyl modified nucleotides; and the even-numbered positions of the antisense strand comprise 2′flouro-modified nucleotides and unmodified deoxyribonucleotides. In some embodiments, there are not three 2′ fluoro-modified purines in a row. In some embodiments, there are not three 2′ fluoro-modified pyrimidines in a row.

In some embodiments, position nine of the sense strand comprises an unmodified deoxyribonucleotide. In some embodiments, positions 5, 7, and 8 of the sense strand comprise 2′fluoro-modified nucleotides. In some embodiments, all pyrimidines in positions 10 to 21 of the sense strand comprise 2′-O-methyl modified pyrimidines and all purines in positions 10 to 21 of the comprise 2′-O-methyl modified purines or 2′fluoro-modified purines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all pyrimidines in positions 10 to 21 of the sense strand comprise 2′-O-methyl modified pyrimidines and all purines in positions 10 to 21 of the comprise 2′-O-methyl modified purines or 2′fluoro-modified purines; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2′fluoro-modified nucleotides. In some embodiments, all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines; the odd-numbered positions of the antisense strand comprise 2′-O-methyl modified nucleotides; and the even-numbered positions of the antisense strand comprise 2′flouro-modified nucleotides and unmodified deoxyribonucleotide.

In some embodiments, the moiety includes a negatively charged group attached at a 5′ end of the oligonucleotide. This may be referred to as a 5′-end group. In some embodiments, the negatively charged group is attached at a 5′ end of an antisense strand of an siRNA disclosed herein. The 5′-end group may be or include a 5′-end phosphorothioate, 5′-end phosphorodithioate, 5′-end vinylphosphonate (5′-VP), 5′-end methylphosphonate, 5′-end cyclopropyl phosphonate, or a 5′-deoxy-5′-C-malonyl. The 5′-end group may comprise 5′-VP. In some embodiments, the 5′-VP comprises a trans-vinylphosphate or cis-vinylphosphate. The 5′-end group may include an extra 5′ phosphate. A combination of 5′-end groups may be used.

In some embodiments, the oligonucleotide includes a negatively charged group. The negatively charged group may aid in cell or tissue penetration. The negatively charged group may be attached at a 5′ or 3′ end (e.g. a 5′ end) of the oligonucleotide. This may be referred to as an end group. The end group may be or include a phosphorothioate, phosphorodithioate, vinylphosphonate, methylphosphonate, cyclopropyl phosphonate, or a deoxy-C-malonyl. The end group may include an extra 5′ phosphate such as an extra 5′ phosphate. A combination of end groups may be used.

In some embodiments, the oligonucleotide includes a phosphate mimic. In some embodiments, the phosphate mimic comprises vinyl phosphonate. In some embodiments, the vinyl phosphonate comprises a trans-vinylphosphate. In some embodiments, the vinyl phosphonate comprises a cis-vinylphosphate. An example of a nucleotide that includes a vinyl phosphonate is shown below.

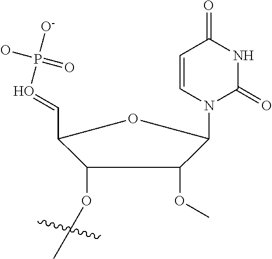

### 5 vinylphosphonate 2′ O Methyl Uridine

In some embodiments, the vinyl phosphonate increases the stability of the oligonucleotide. In some embodiments, the vinyl phosphonate increases the accumulation of the oligonucleotide in tissues. In some embodiments, the vinyl phosphonate protects the oligonucleotide from an exonuclease or a phosphatase. In some embodiments, the vinyl phosphonate improves the binding affinity of the oligonucleotide with the siRNA processing machinery.

In some embodiments, the oligonucleotide includes 1 vinyl phosphonate. In some embodiments, the oligonucleotide includes 2 vinyl phosphonates. In some embodiments, the oligonucleotide includes 3 vinyl phosphonates. In some embodiments, the oligonucleotide includes 4 vinyl phosphonates. In some embodiments, the antisense strand of the oligonucleotide comprises a vinyl phosphonate at the 5′ end. In some embodiments, the antisense strand of the oligonucleotide comprises a vinyl phosphonate at the 3′ end. In some embodiments, the sense strand of the oligonucleotide comprises a vinyl phosphonate at the 5′ end. In some embodiments, the sense strand of the oligonucleotide comprises a vinyl phosphonate at the 3′ end.

### 1. Hydrophobic Moieties

In some embodiments, the composition comprises an oligonucleotide that inhibits the expression of GPAM, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of GPAM, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof.

In some embodiments, the composition comprises an oligonucleotide that inhibits the expression of GPAM, wherein the oligonucleotide comprises a hydrophobic ligand or moiety. In some embodiments, the hydrophobic ligand or moiety comprises cholesterol. In some embodiments, the hydrophobic ligand or moiety comprises a cholesterol derivative. In some embodiments, the hydrophobic ligand or moiety is attached at a 3′ terminus of the oligonucleotide. In some embodiments, the hydrophobic ligand or moiety s attached at a 5′ terminus of the oligonucleotide. In some embodiments, the composition comprises a sense strand, and the hydrophobic ligand or moiety is attached to the sense strand (e.g. attached to a 5′ end of the sense strand, or attached to a 3′ end of the sense strand). In some embodiments, the composition comprises an antisense strand, and the hydrophobic ligand or moiety is attached to the antisense strand (e.g. attached to a 5′ end of the antisense strand, or attached to a 3′ end of the antisense strand). In some embodiments, the composition comprises a hydrophobic ligand or moiety attached at a 3′ or 5′ terminus of the oligonucleotide.

In some embodiments, a hydrophobic moiety is attached to the oligonucleotide (e.g. a sense strand and/or an antisense strand of a siRNA). In some embodiments, a hydrophobic moiety is attached at a 3′ terminus of the oligonucleotide. In some embodiments, a hydrophobic moiety is attached at a 5′ terminus of the oligonucleotide. In some embodiments, the hydrophobic moiety comprises cholesterol. In some embodiments, the hydrophobic moiety includes a cyclohexanyl.

In some embodiments, the composition comprises an oligonucleotide that inhibits the expression of GPAM, wherein the oligonucleotide comprises a lipid attached at a 3′ or 5′ terminus of the oligonucleotide. In some embodiments, a lipid is attached at a 3′ terminus of the oligonucleotide. In some embodiments, a lipid is attached at a 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof. In some embodiments, the lipid comprises stearyl, lithocholyl, docosanyl, docosahexaenyl, or myristyl. In some embodiments, the lipid comprises cholesterol. In some embodiments, the lipid includes a sterol such as cholesterol. In some embodiments, the lipid comprises stearyl, t-butylphenol, n-butylphenol, octylphenol, dodecylphenol, phenyl n-dodecyl, octadecylbenzamide, hexadecylbenzamide, or octadecylcyclohexyl. In some embodiments, the lipid comprises phenyl para C12.

In some embodiments, the oligonucleotide comprises any aspect of the following structure:

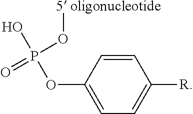

In some embodiments, the oligonucleotide comprises any aspect of the following structure:

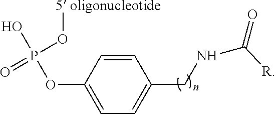

In some embodiments, the oligonucleotide comprises any aspect of the following structure:

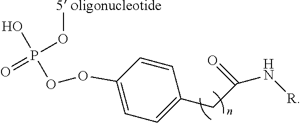

In some embodiments, the oligonucleotide comprises any aspect of the following structure: The aspect included in the oligonucleotide may include the entire structure, or may include the lipid moiety, of any of the structures shown. In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, the alkyl group contains 4-18 carbons. In some embodiments, the lipid moiety comprises an alcohol or ether.

In some embodiments, the lipid includes a fatty acid. In some embodiments, the lipid comprises a lipid depicted in Table 1. The example lipid moieties in Table 1 are shown attached at a 5′ end of an oligonucleotide, in which the 5′ terminal phosphate of the oligonucleotide is shown with the lipid moiety. In some embodiments, a lipid moiety in Table 1 may be attached at a different point of attachment than shown. For example, the point of attachment of any of the lipid moieties in the table may be at a 3′ oligonucleotide end. In some embodiments, the lipid is used for targeting the oligonucleotide to a non-hepatic cell or tissue.

TABLE 1
Hydrophobic moiety examples

| Hydro- | Hydro- | |
|---|---|---|
| phobic | phobic | |
| Moiety | Moiety | |
| Description | Name | Example Conjugation |
| stearyl | ETL3 | 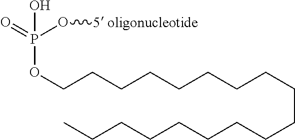 |
| t-butylphenyl | ETL7 | 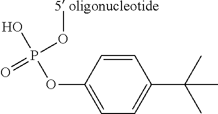 |
| n-butylphenyl | ETL8 | 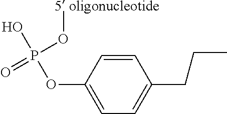 |
| octylphenyl | ETL9 | 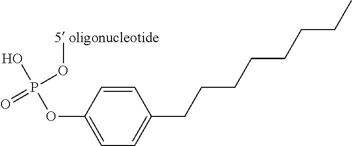 |
| dodecylphenyl | ETL10 | 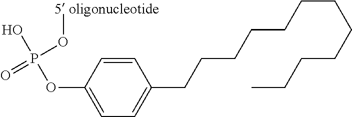 |
| phenyl n-dodecyl | ETL12 | 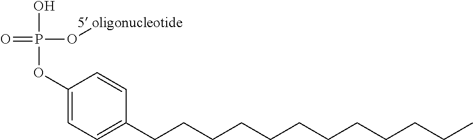 |
| octadecyl- benzamide | ETL13 | 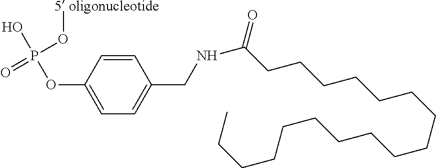 |
| hexadecyl- benzamide | ETL15 | 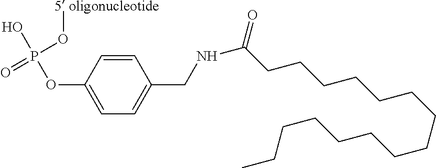 |
| octadecyl- cyclohexyl | ETL16 | 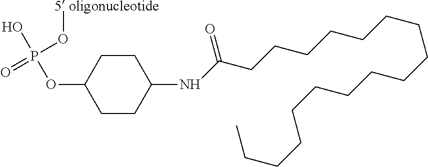 |
| myristamido methylphenyl | ETL18 | 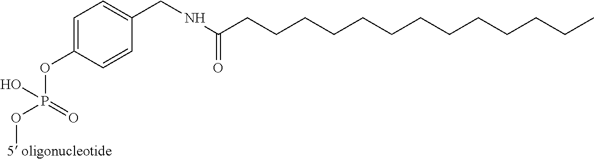 |
| lauramido methylphenyl | ETL19 | 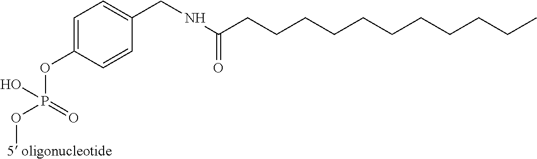 |
| phenethyl- palmityl | ETL20 | 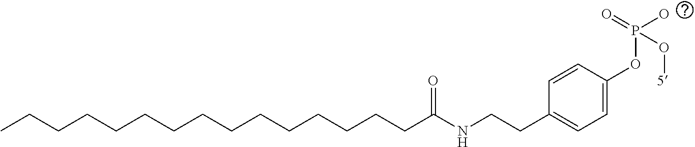 |

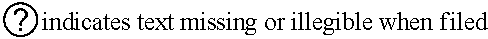

In some embodiments, the lipid or lipid moiety includes 16 to 18 carbons. In some embodiments, the lipid includes 16 carbons. In some embodiments, the lipid includes 17 carbons. In some embodiments, the lipid includes 18 carbons. In some embodiments, the lipid moiety includes 16 carbons. In some embodiments, the lipid moiety includes 17 carbons. In some embodiments, the lipid moiety includes 18 carbons.

The hydrophobic moiety may include a linker that comprises a carbocycle. The carbocycle may be six-membered. Some examples of a carbocycle include phenyl or cyclohexyl. The linker may include a phenyl. The linker may include a cyclohexyl. The lipid may be attached to the carbocycle, which may in turn be attached at a phosphate (e.g. 5′ or 3′ phosphate) of the oligonucleotide. In some embodiments, the lipid or hydrocarbon, and the end of the sense are connected to the phenyl or cyclohexyl linker in the 1,4; 1,3; or 1,2 substitution pattern (e.g. the pama, meta, or ortho phenyl configuration). In some embodiments, the lipid or hydrocarbon, and the end of the sense are connected to the phenyl or cyclohexyl linker in the 1,4 substitution pattern (e.g. the para phenyl configuration). The lipid may be attached to the carbocycle in the 1,4 substitution pattern relative to the oligonucleotide. The lipid may be attached to the carbocycle in the 1,3 substitution pattern relative to the oligonucleotide. The lipid may be attached to the carbocycle in the 1,2 substitution pattern relative to the oligonucleotide. The lipid may be attached to the carbocycle in the ortho orientation relative to the oligonucleotide. The lipid may be attached to the carbocycle in the para orientation relative to the oligonucleotide. The lipid may be attached to the carbocycle in the meta orientation relative to the oligonucleotide.

The lipid moiety may comprise or consist of the following structure

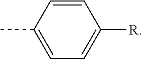

In some embodiments, the lipid moiety comprises or consists of the following structure:

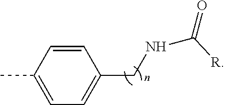

In some embodiments, the lipid moiety comprises the following structure:

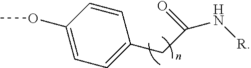

In some embodiments, the lipid moiety comprises or consist of the following structure:

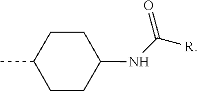

In some embodiments, the dotted line indicates a covalent connection. The covalent connection may between an end of the sense or antisense strand. For example, the connection may be to the 5′ end of the sense strand. In some embodiments, n is 0-3. In some embodiments, n is 1-3. In some embodiments, n is 0. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, or 10. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons.

The lipid moiety may be attached at a 5′ end of the oligonucleotide. The 5′ end may have one phosphate linking the lipid moiety to a 5′ carbon of a sugar of the oligonucleotide. The 5′ end may have two phosphates linking the lipid moiety to a 5′ carbon of a sugar of the oligonucleotide. The 5′ end may have three phosphates linking the lipid moiety to a 5′ carbon of a sugar of the oligonucleotide. The 5′ end may have one phosphate connected to the 5′ carbon of a sugar of the oligonucleotide, where the one phosphate is connected to the lipid moiety. The 5′ end may have two phosphates connected to the 5′ carbon of a sugar of the oligonucleotide, where the one of the two phosphates is connected to the lipid moiety. The 5′ end may have three phosphates connected to the 5′ carbon of a sugar of the oligonucleotide, where the one of the three phosphates is connected to the lipid moiety. The sugar may include a ribose. The sugar may include a deoxyribose. The sugar may be modified a such as a 2′ modified sugar (e.g. a 2′ O-methyl or 2′ fluoro ribose). A phosphate of the 5′ end may include a modification such as a sulfur in place of an oxygen. Two phosphates of the 5′ end may include a modification such as a sulfur in place of an oxygen. Three phosphates of the 5′ end may include a modification such as a sulfur in place of an oxygen.

In some embodiments, the oligonucleotide includes 1 lipid moiety. In some embodiments, the oligonucleotide includes 2 lipid moieties. In some embodiments, the oligonucleotide includes 3 lipid moieties. In some embodiments, the oligonucleotide includes 4 lipid moieties.

Some embodiments relate to a method of making an oligonucleotide comprising a hydrophobic conjugate. A strategy for making hydrophobic conjugates may include use of a phosphoramidite reagent based upon a 6-membered ring alcohol such as a phenol or cyclohexanol. The phosphoramidite may be reacted to a nucleotide to connect the nucleotide to the hydrophobic moiety, and thereby produce the hydrophobic conjugate. Some examples of phosphoramidite reagents that may be used to produce a hydrophobic conjugate are provided as follows:

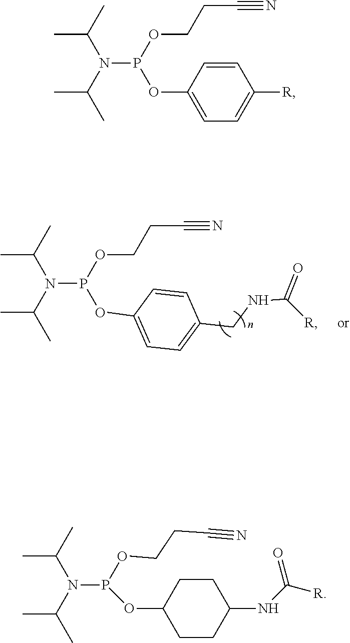

In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons. Any one of the phosphoramidite reagents may be reacted to a 5′ end of an oligonucleotide to produce an oligonucleotide comprising a hydrophobic moiety. In some embodiments, the phosphoramidite reagents is reacted to a 5′ end of a sense strand of an siRNA. The sense strand may then be hybridized to an antisense strand to form a duplex. The hybridization may be performed by incubating the sense and antisense strands in solution at a given temperature. The temperature may be gradually reduced. The temperature may comprise or include a temperature comprising an annealing temperature for the sense and antisense strands. The temperature may be below or include a temperature below the annealing temperature for the sense and antisense strands. The temperature may be below a melting temperature of the sense and antisense strands.

The lipid may be attached to the oligonucleotide by a linker. The linker may include a polyethyleneglycol (e.g. tetraethyleneglycol).

The modifications described herein may be useful for delivery to a cell or tissue, for example, extrahepatic delivery or targeting of an oligonucleotide composition. The modifications described herein may be useful for targeting an oligonucleotide composition to a cell or tissue.

### 2. Sugar Moieties

In some embodiments, the composition comprises an oligonucleotide that inhibits the expression of GPAM, wherein the oligonucleotide comprises a sugar moiety. The sugar moiety may include an N-acetyl galactose moiety (e.g. an N-acetylgalactosamine (GalNAc) moiety), an N-acetyl glucose moiety (e.g. an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages when they target or bind a mannose receptor such as CD206. The sugar moiety may be useful for binding or targeting an asialoglycoprotein receptor such as an asialoglycoprotein receptor of a hepatocyte. The GalNAc moiety may bind to an asialoglycoprotein receptor. The GalNAc moiety may target a hepatocyte.

In some embodiments, the composition comprises an oligonucleotide that inhibits the expression of GPAM, wherein the oligonucleotide comprises an N-acetylgalactosamine (GalNAc) moiety. GalNAc may be useful for hepatocyte targeting. The GalNAc moiety may include a bivalent or trivalent branched linker. The oligo may be attached to 1, 2 or 3 GalNAcs through a bivalent or trivalent branched linker. The GalNAc moiety may include 1, 2, 3, or more GalNAc molecules. The GalNAc moiety may be attached at a 3′ or 5′ terminus of the oligonucleotide.

In some embodiments, the composition comprises an oligonucleotide that inhibits the expression of GPAM, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

Disclosed herein, in some embodiments, are compositions comprising an oligonucleotide that inhibits the expression of GPAM, wherein the oligonucleotide comprises a GalNAc moiety. The GalNAc moiety may be included in any formula, structure, or GalNAc moiety shown below. In some embodiments, described herein is a compound (e.g. oligonucleotide) represented by Formula (I) or (II):

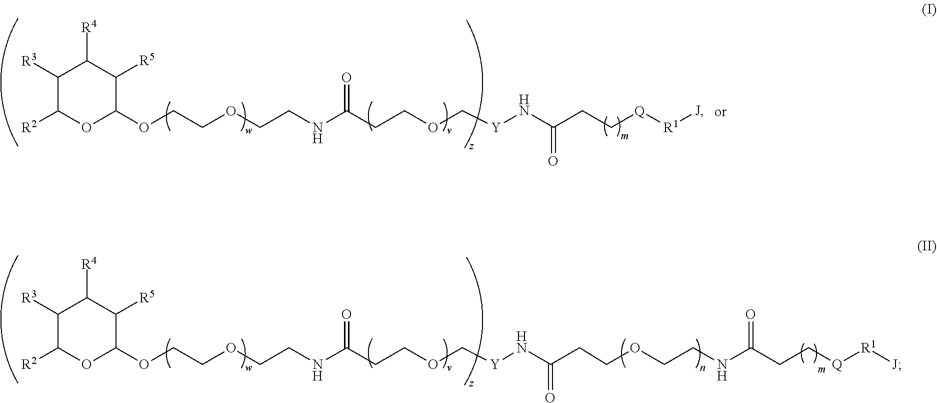

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
   if z is 3, Y is C
   if z is 2, Y is CR⁶, or
   if z is 1, Y is C(R⁶)₂;
Q is selected from:
   C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂;
R¹ is a linker selected from:
   —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from:
   C₁₋₆ alkyl optionally substituted with one or more substituents independently selected from halogen, —OR⁷, —SR⁷, —N(R²)₂, —C(O)R⁷, —C(O)N(R⁷)₂, —N(R²)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
R³ and R⁴ are each independently selected from:
   —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from:
   —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
   hydrogen;
   halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
   C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃0.10 carbocycle, 3- to 10-membered heterocycle, and C₁₋₆ haloalkyl.
      In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is selected from 1 and 2. In some embodiments, z is 3 and Y is C. In some embodiments, Q is selected from C₅₋₆ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, Q is selected from C₅₋₆ carbocycle optionally substituted with one or more substituents independently selected from halogen, —CN, —OH, —SH, —NO₂, and —NH₂. In some embodiments, Q is selected from phenyl and cyclohexyl, each of which is optionally substituted with one or more substituents independently selected from halogen, —CN, —OH, —SH, —NO₂, and —NH₂. In some embodiments, Q is selected from phenyl. In some embodiments, Q is selected from cyclohexyl. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂—NR². In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, and —OP(OR⁷)O—. In some embodiments, R¹ is selected from —OP(O)(OR⁷)O—, —OP(S)(OR⁷)O—, —OP(O)(O⁻)O—, —OP(S)(O⁻)O—, —OP(O)(S⁻)O—, and —OP(OR⁷)O—. In some embodiments, R¹ is selected from —OP(O)(OR⁷)O— and —OP(OR⁷)O—. In some embodiments, R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, R² is selected from C₁₋₃ alkyl substituted with one or more substituents independently selected from —OR⁷, —OC(O)R⁷, —SR⁷, and —N(R⁷)₂. In some embodiments, R² is selected from C₁₋₃ alkyl substituted with one or more substituents independently selected from —OR⁷ and —OC(O)R⁷. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from —OR⁷, —SR⁷, —OC(O)R⁷, and —N(R⁷)₂. In some embodiments, R³ is selected from —OR⁷— and —OC(O)R⁷. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from —OR⁷, —SR⁷, —OC(O)R⁷, and —N(R⁷)₂. In some embodiments, R⁴ is selected from —OR⁷— and —OC(O)R⁷. In some embodiments, R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, R⁵ is selected from —OC(O)R⁷ and —N(R⁷)C(O)R⁷. In some embodiments, each R⁷ is independently selected from: hydrogen; and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from C₁₋₆ alkyl optionally substituted with one or more substituents independently selected from halogen, —CN, —OH, and —SH. In some embodiments, w is 1; v is 1; n is 2; m is 1 or 2; z is 3 and Y is C; Q is phenyl or cyclohexyl, each of which is optionally substituted with one or more substituents independently selected from halogen, —CN, —OH, —SH, —NO₂, —NH₂, and C₁₋₃ alkyl; R¹ is selected from —OP(O)(OR⁷)O—, —OP(S)(OR⁷)O—, —OP(O)(O⁻)O—, —OP(S)(O⁻)O—, —OP(O)(S⁻)O—, and —OP(OR⁷)O—; R² is C₁ alkyl substituted with —OH or —OC(O)CH₃;
R³ is —OH or —OC(O)CH₃; R⁴ is —OH or —OC(O)CH₃; and R⁵ is —NH(O)CH₃. In some embodiments, the compound comprises:

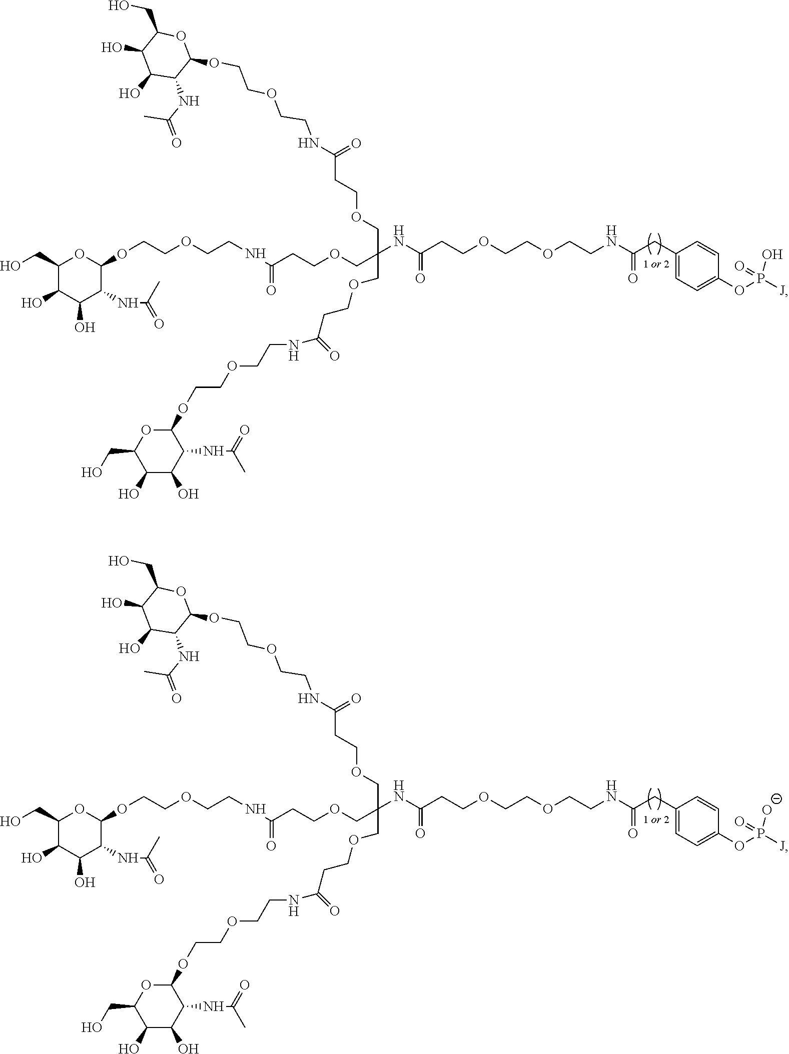

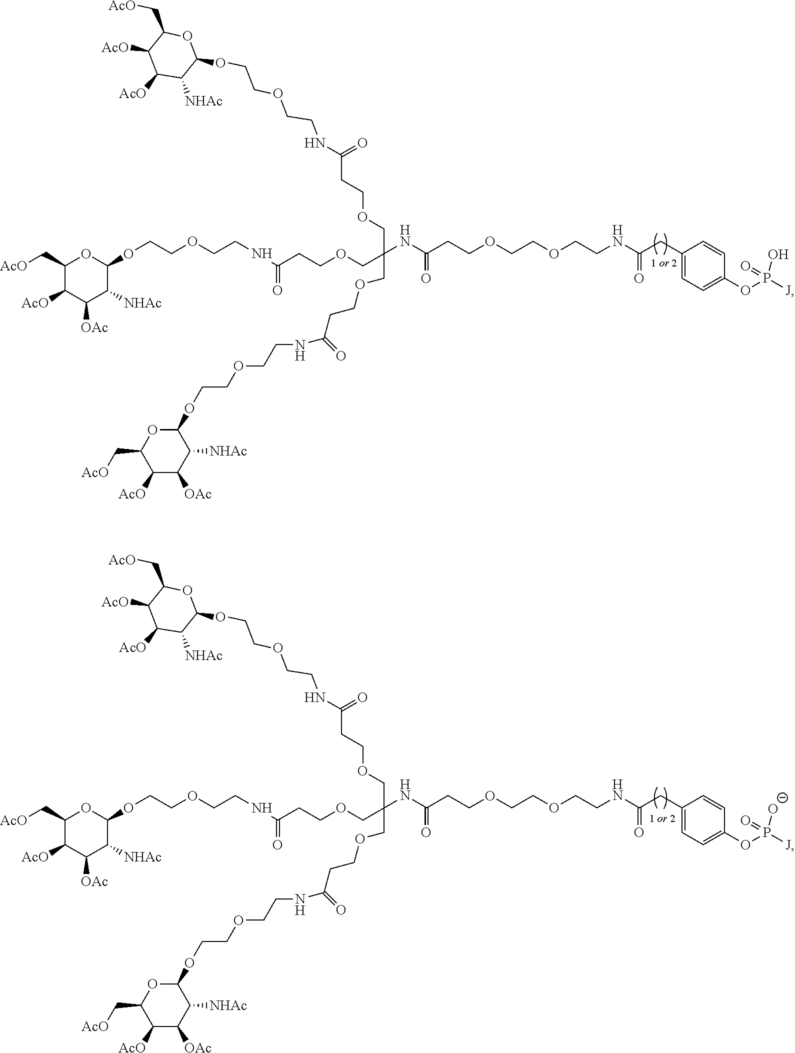

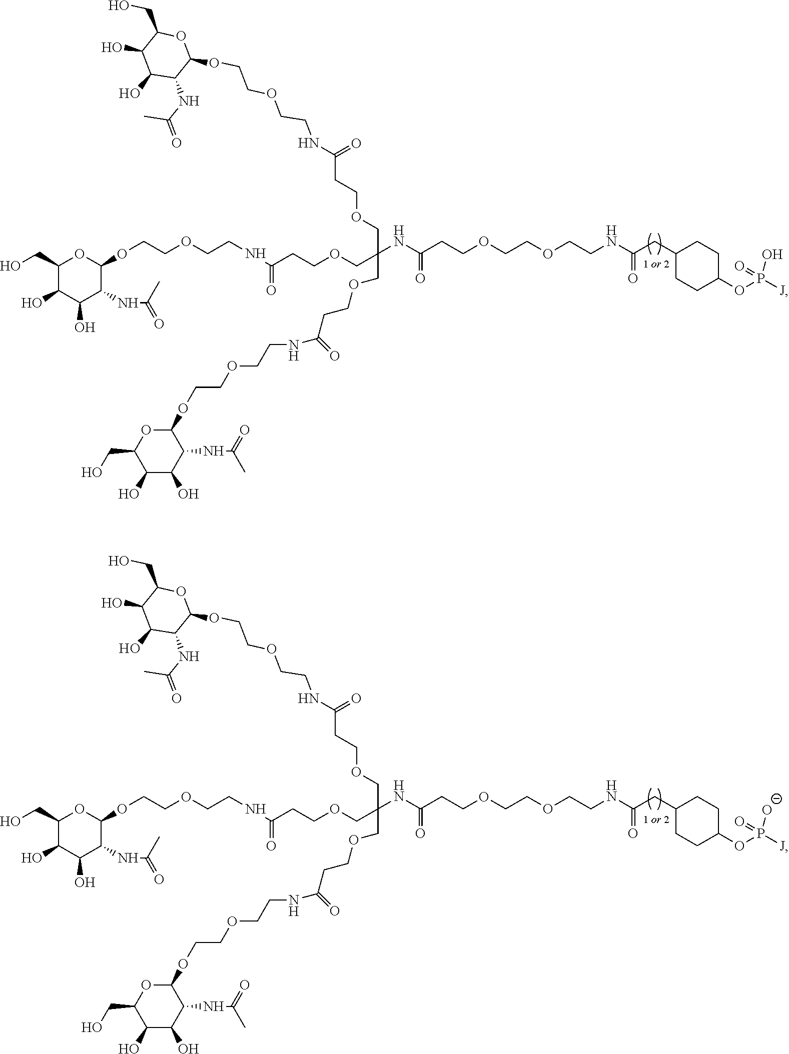

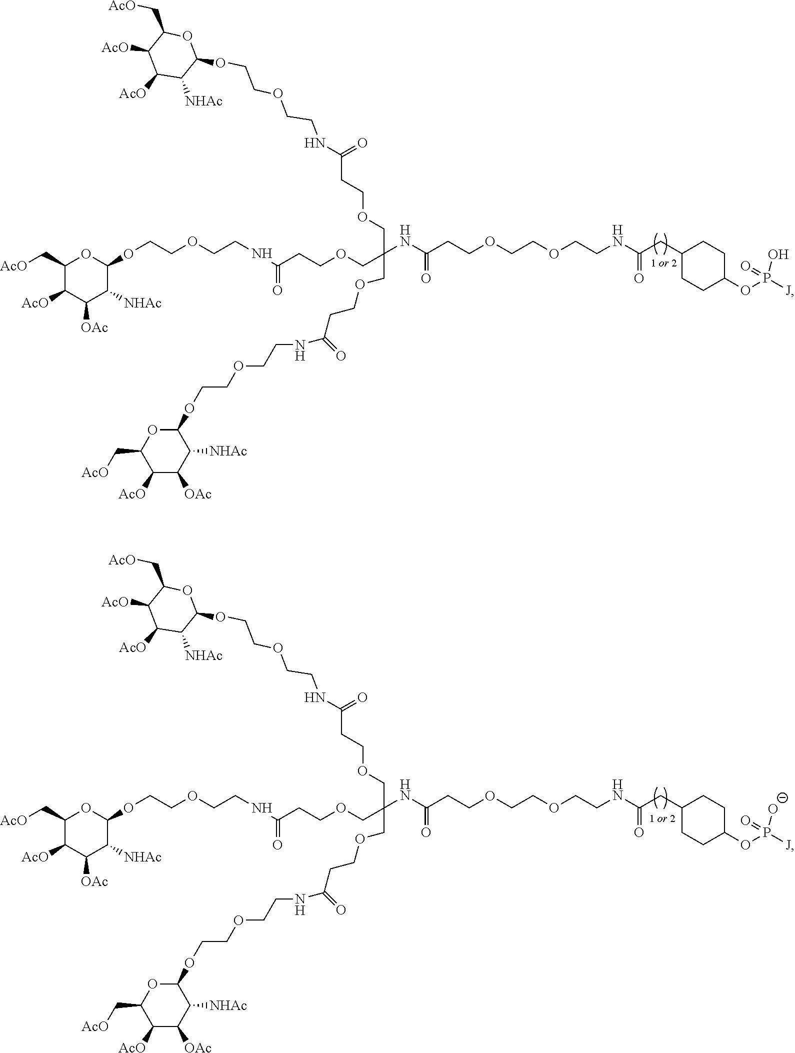

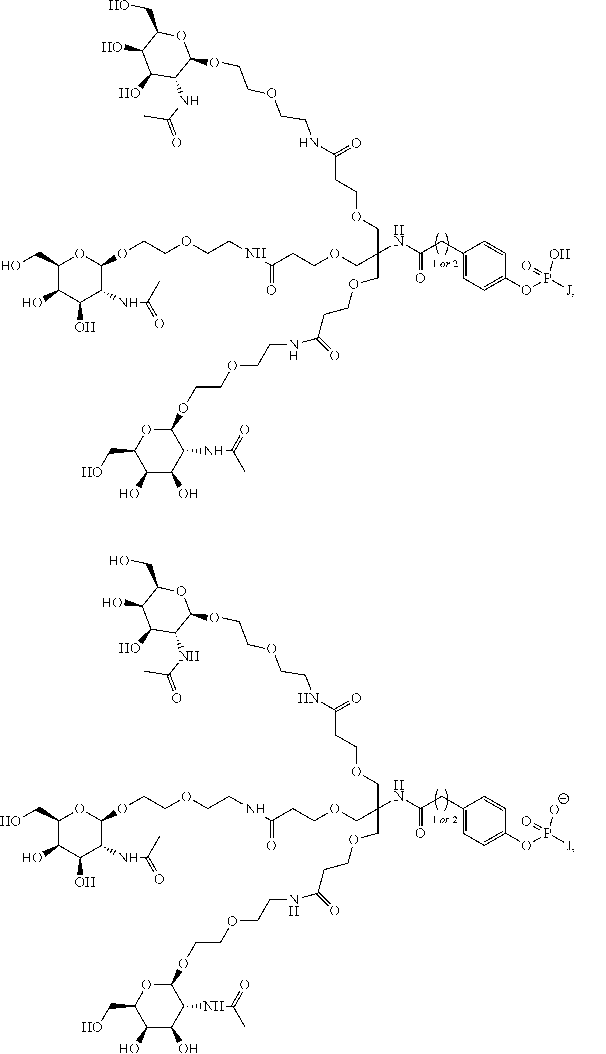

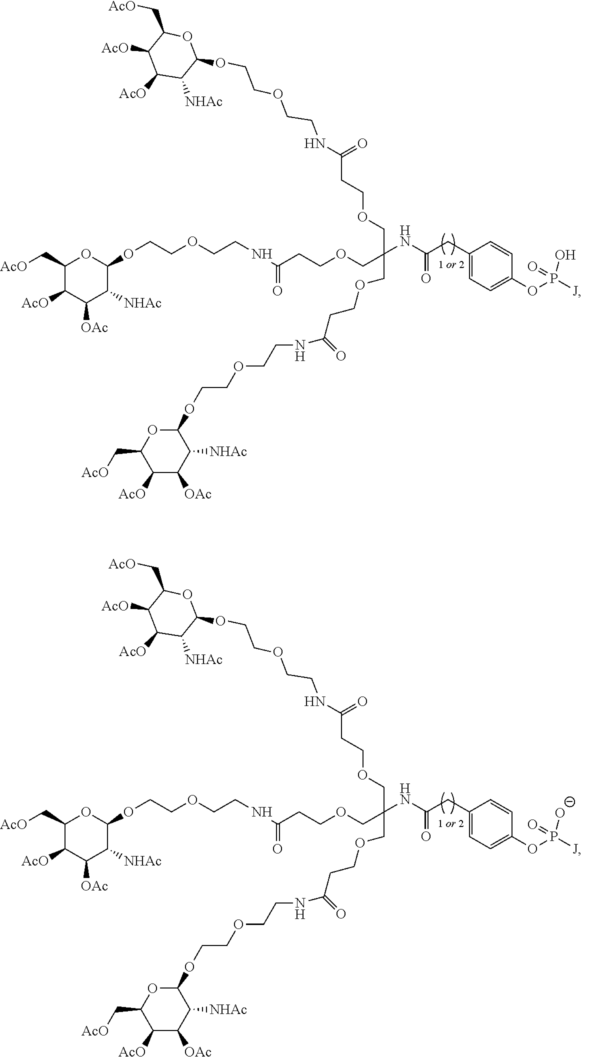

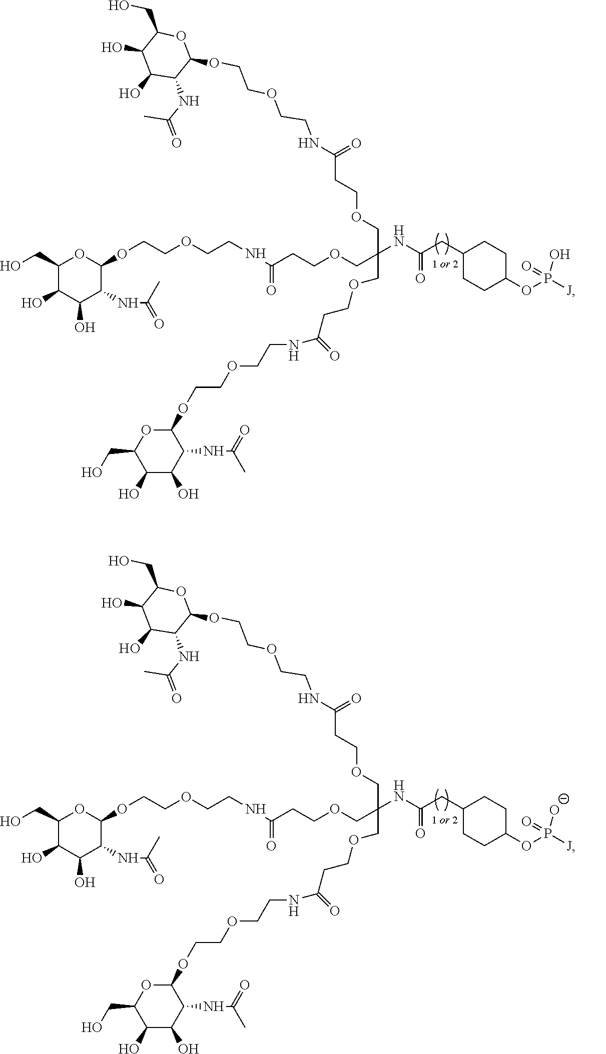

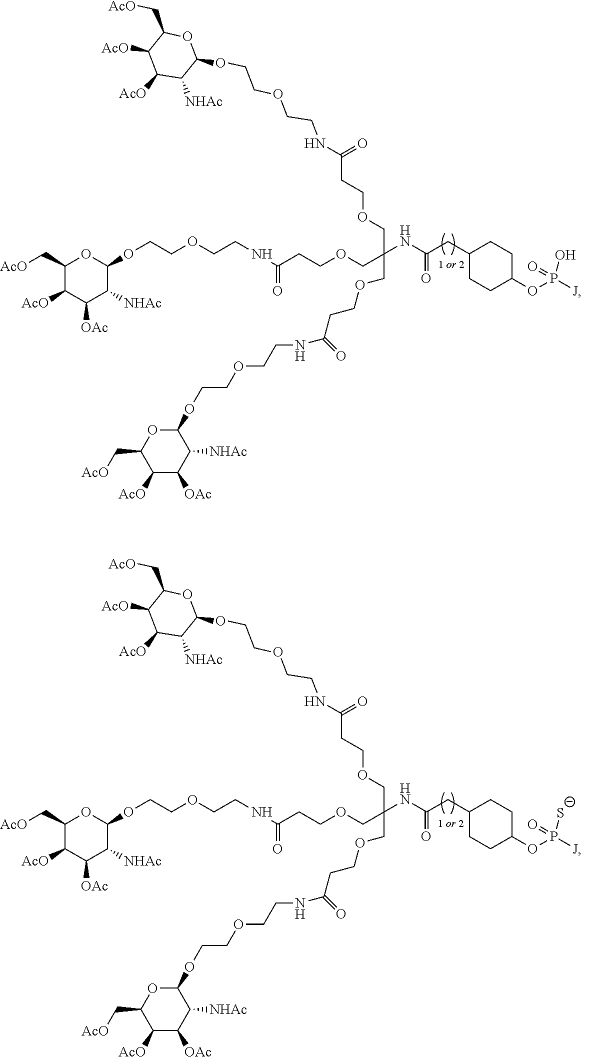

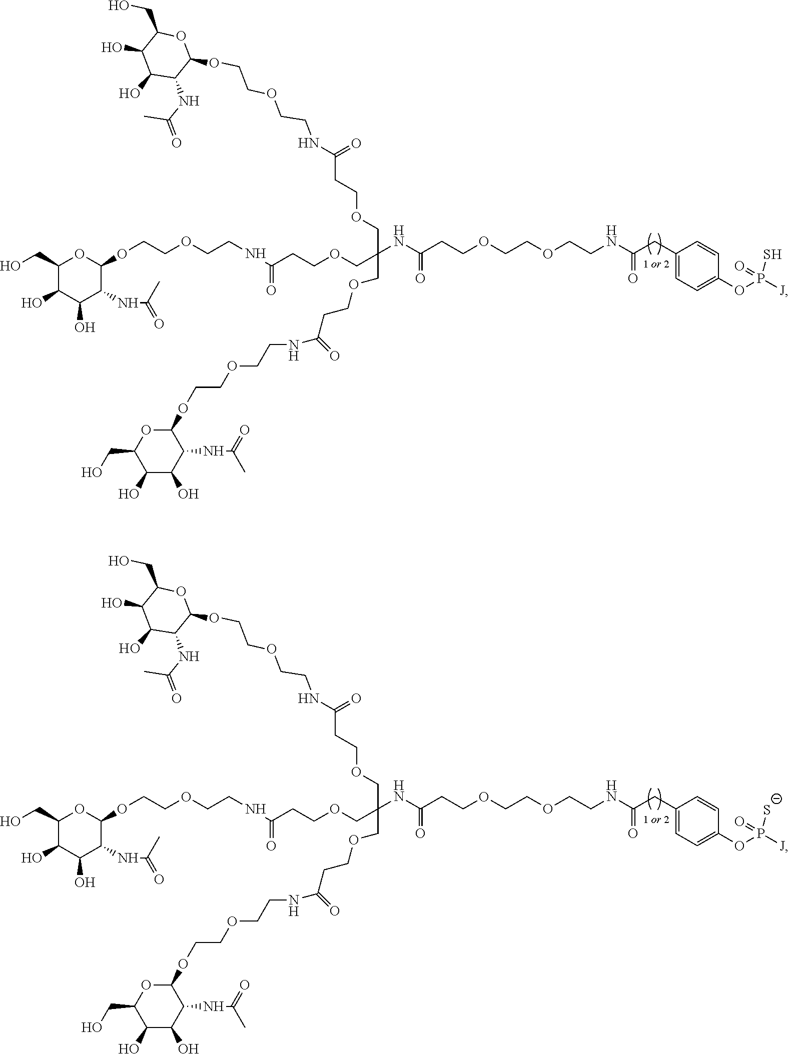

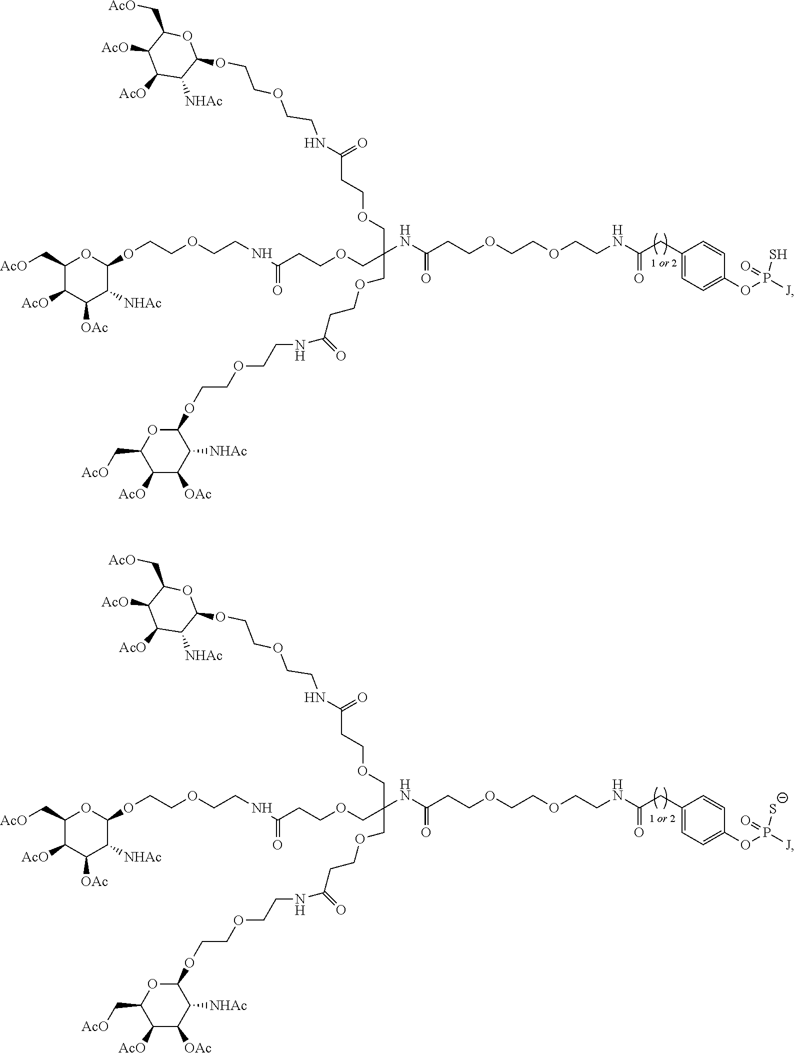

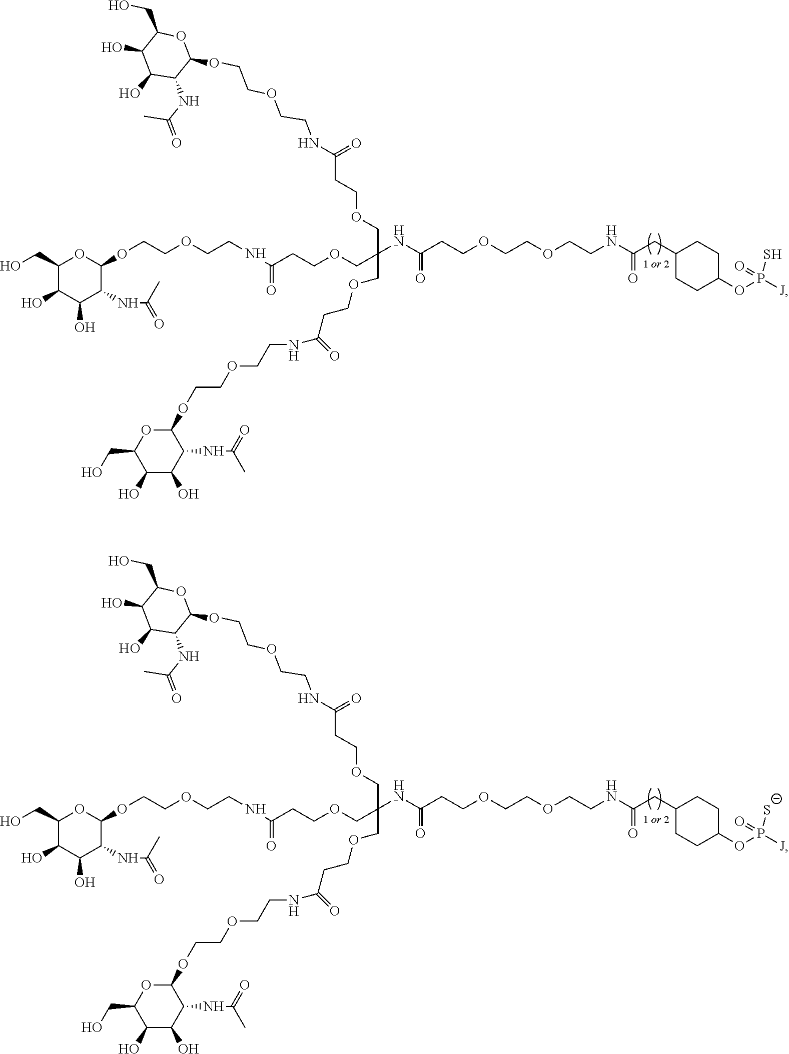

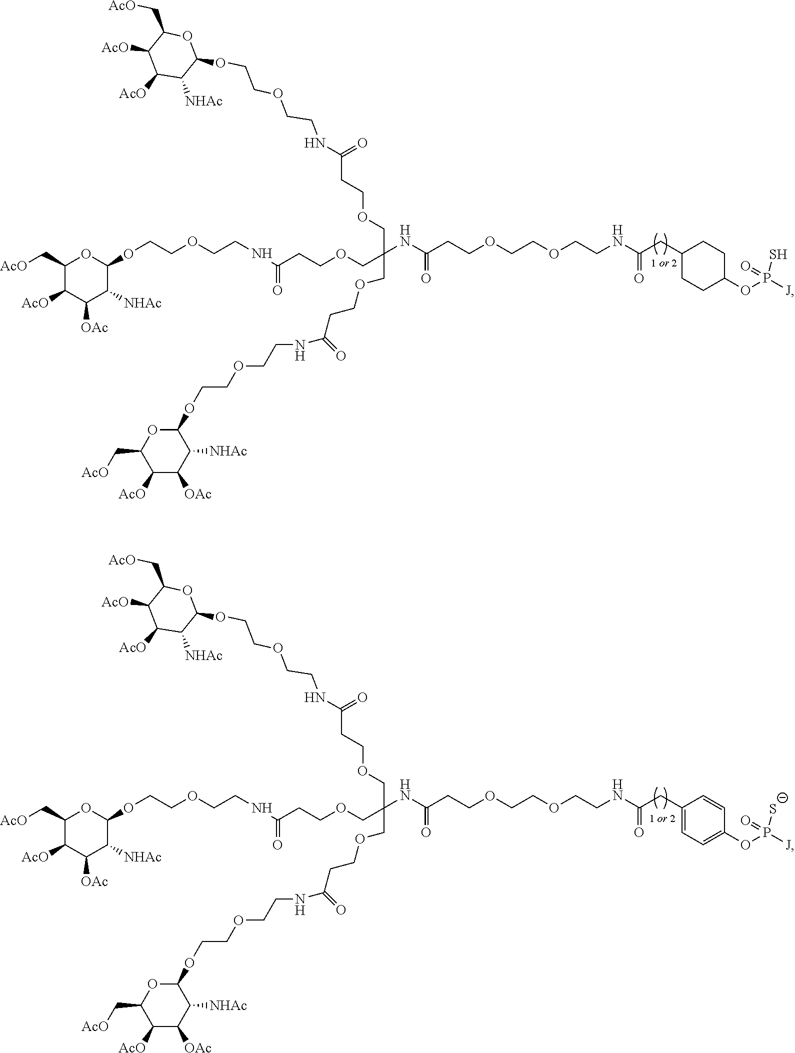

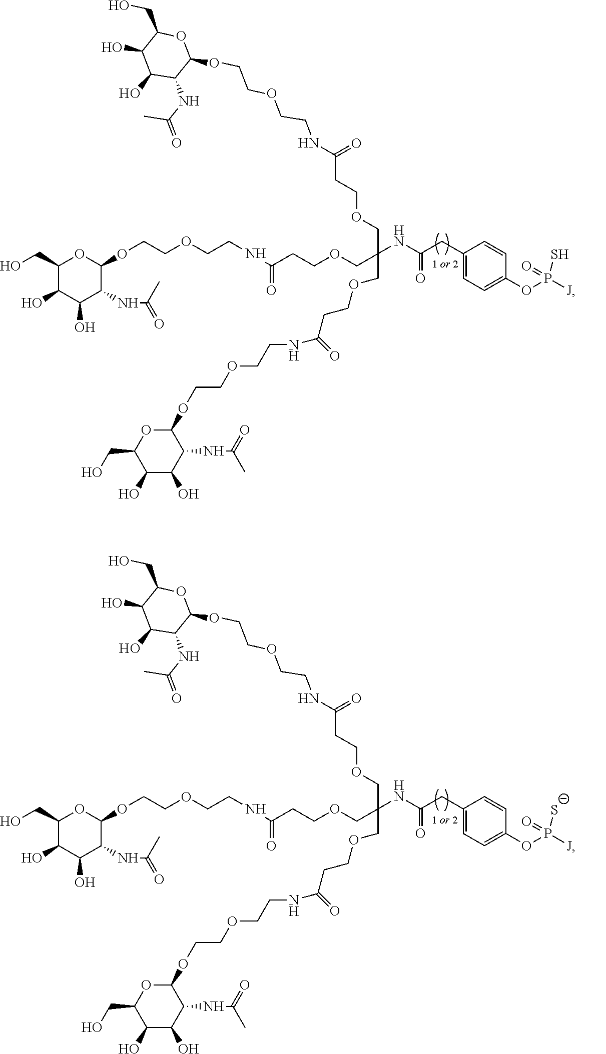

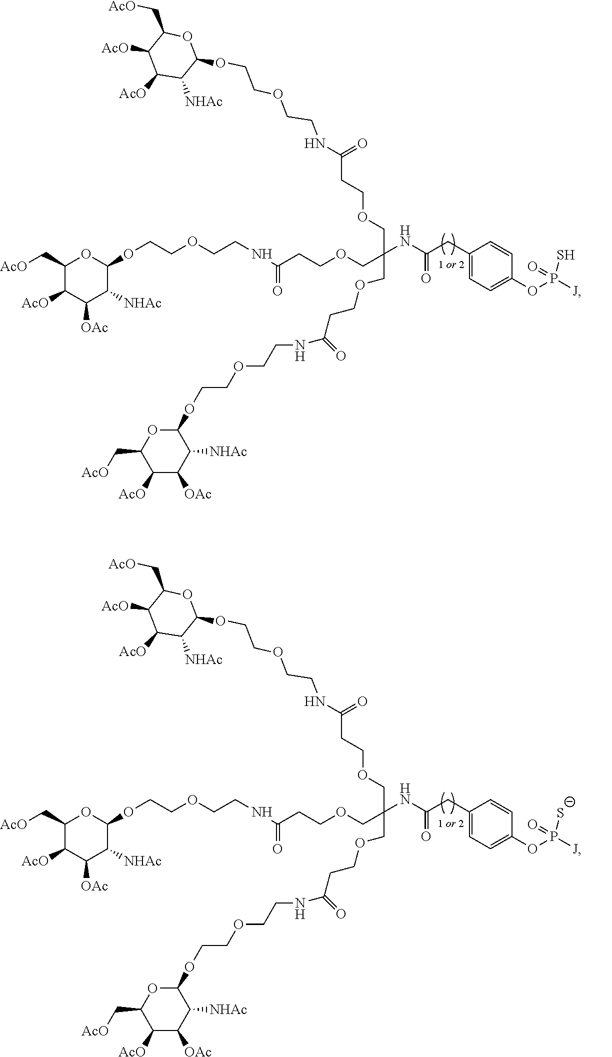

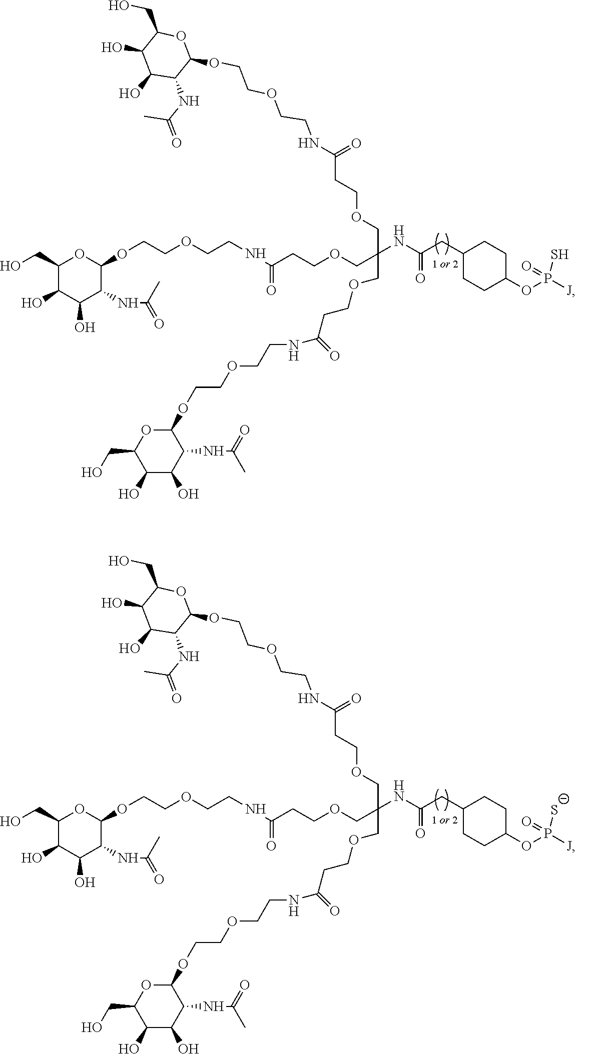

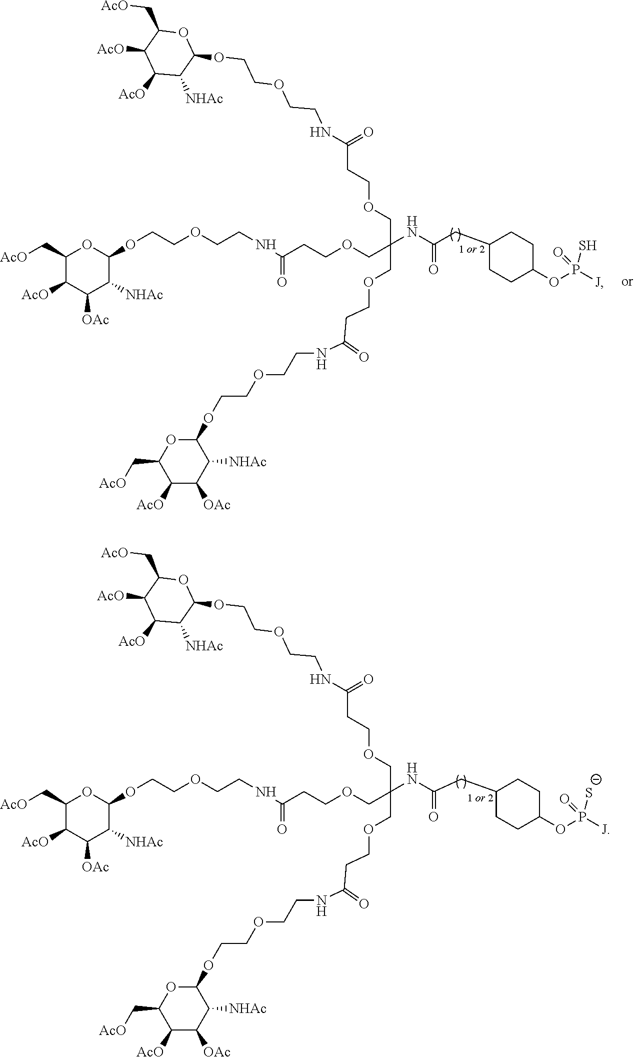

In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the compound binds to an asialoglycoprotein receptor. In some embodiments, the compound targets a hepatocyte.

Some embodiments include the following, where J is the oligonucleotide:

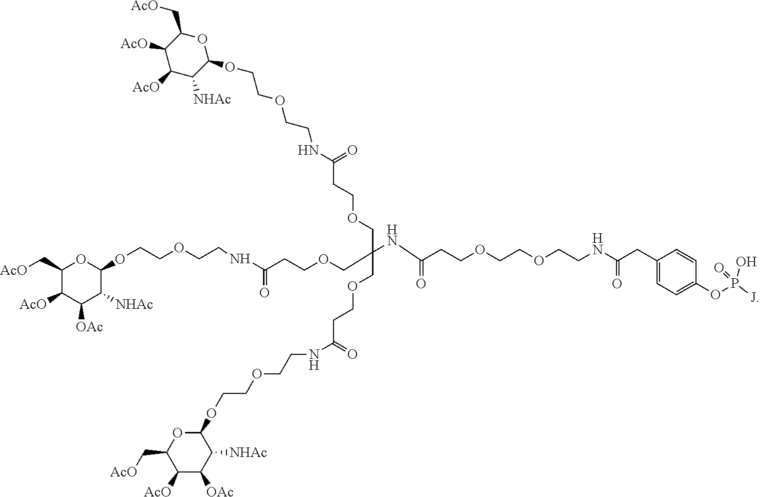

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

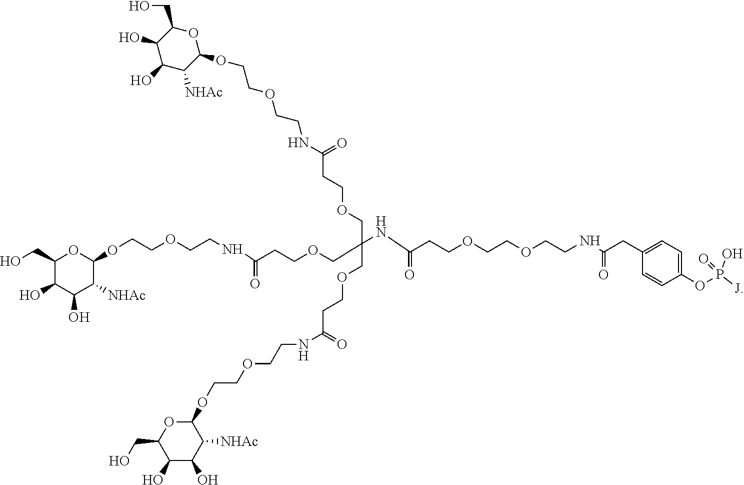

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

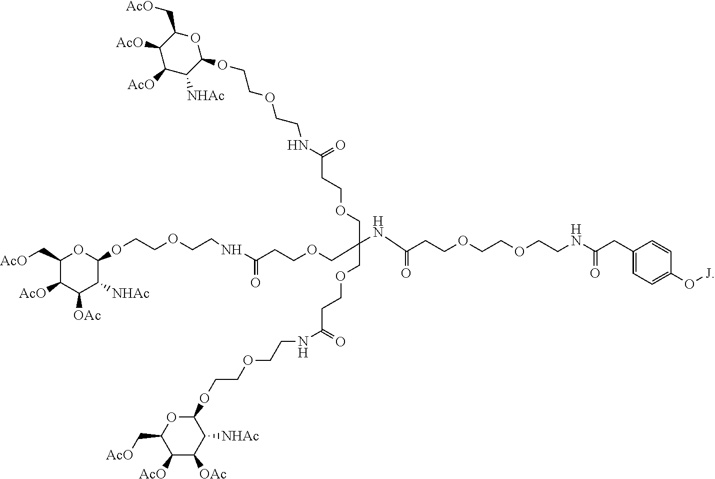

include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

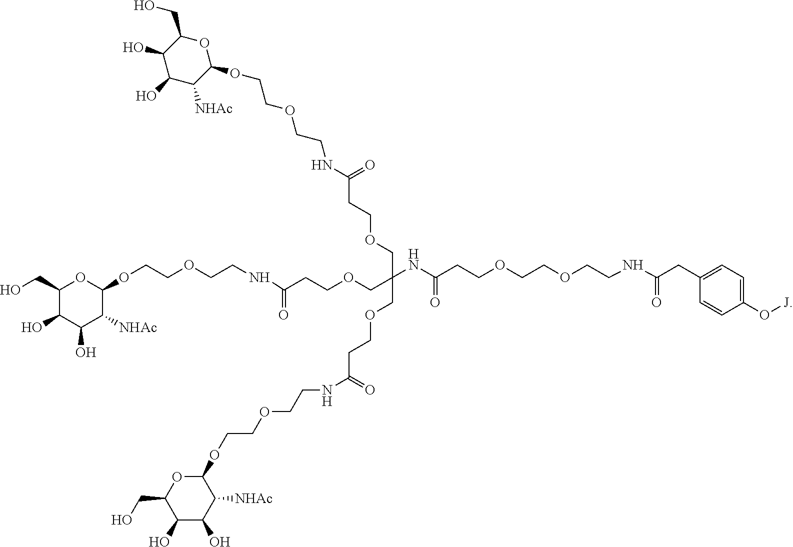

The structure in this compound attached to the oligonucleotide (J) is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

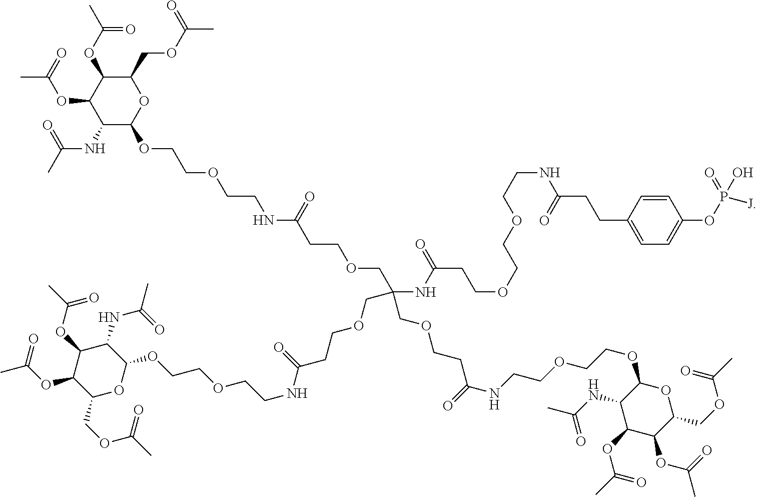

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

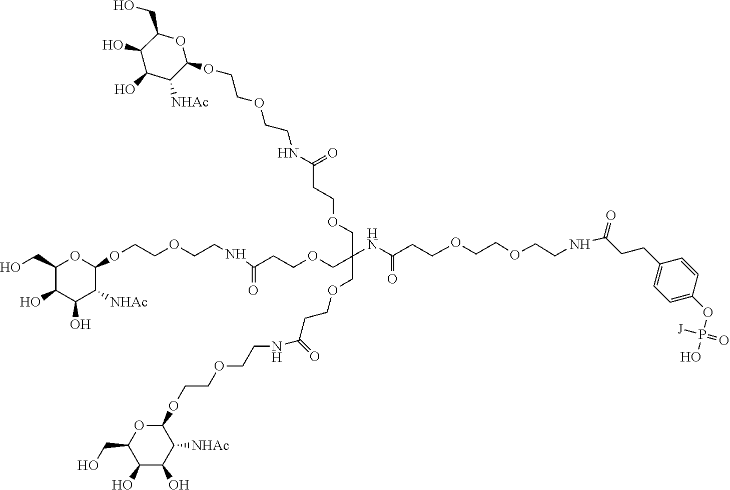

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

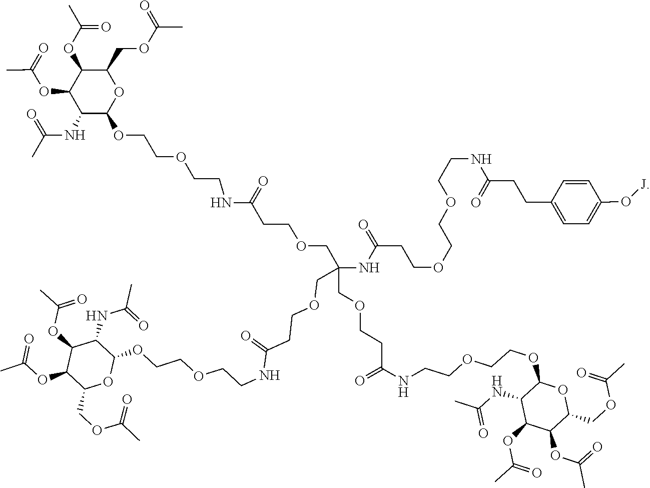

J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.
Some embodiments include the following, where J is the oligonucleotide:

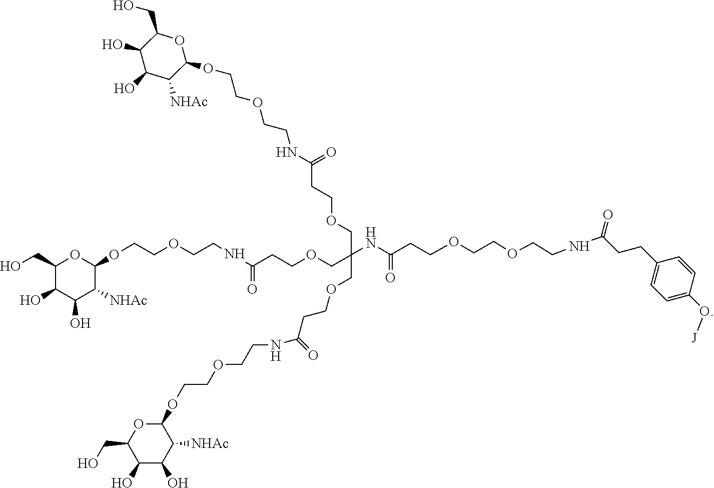

The structure in this compound attached to the oligonucleotide (J) may be referred to as “ETL17,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where the phosphate or “5′” indicates a connection to the oligonucleotide:

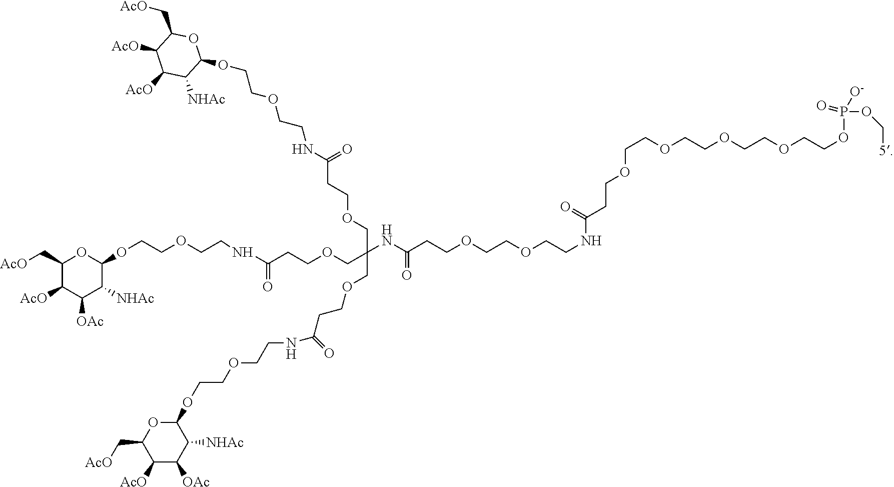

Some embodiments include the following, where the phosphate or “5′” indicates a connection to the oligonucleotide:

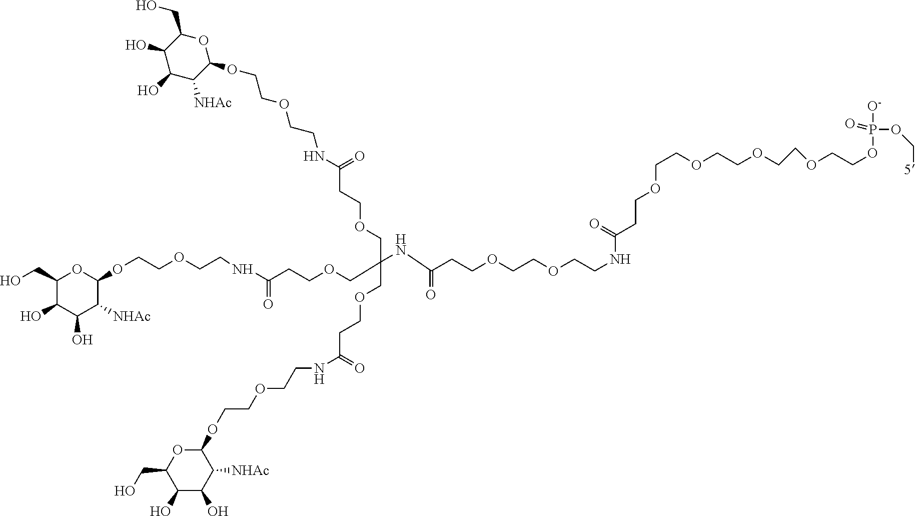

Some embodiments include the following, where J is the oligonucleotide:

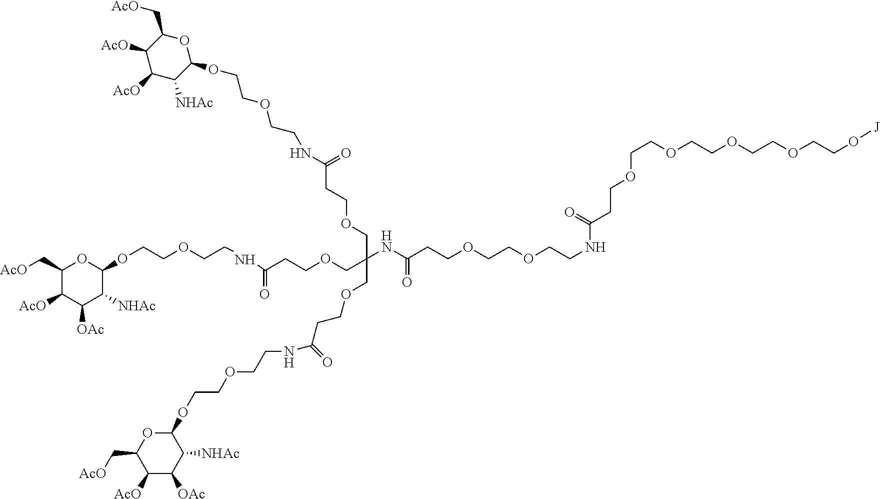

include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

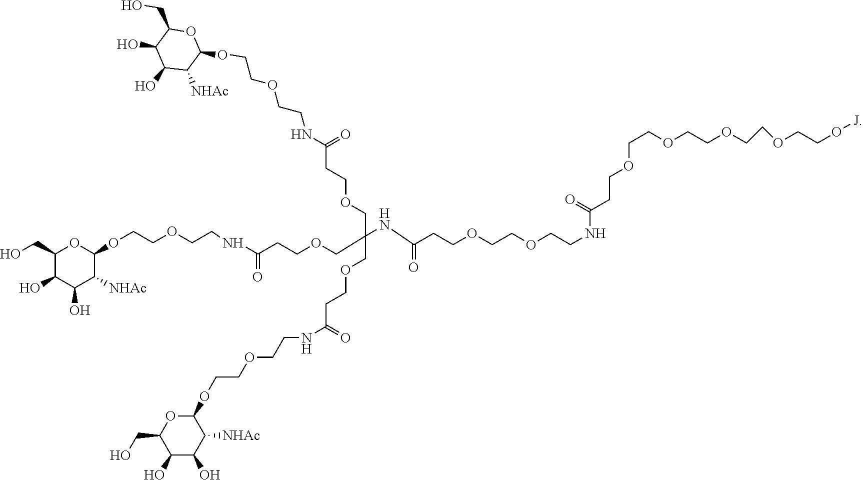

The structure in the compound attached to the oligonucleotide (J) may be referred to as “ETL1,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide
3. Modified siRNAs

In some embodiments, the composition comprises an oligonucleotide that inhibits the expression of GPAM, wherein the oligonucleotide comprises an siRNA comprising a sense strand and an antisense strand, wherein the sense strand comprises modification pattern 1S: 5′-NfsnsNfnNfnNfNfNfnNfnNfnNfnNfnNfsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 3S: 5′-nsnsnnNfnNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-moiety-3′, wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S: 5′-nsnsnnNfnNfNfNfnnnnnnnnnnsnsnN-moiety-3′, wherein “Nf” is a 2′ fluoro-modified nucleoside, “n” is a 2′ O-methyl modified nucleoside, “s” is a phosphorothioate linkage, and N comprises one or more nucleosides. In some embodiments, the moiety in modification pattern 4S or 5S is a lipid moiety. In some embodiments, the moiety in modification pattern 4S or 5S is a sugar moiety. In some embodiments, the sense strand comprises modification pattern 6S: 5′-NfsnsNfnNfnNfnNfnNfnNfnNfnNfnNfsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 7S: 5′-nsnsnnNfNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 8S: 5′-nsnsnnnNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 9S: 5′-nsnsnnnnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 10S: 5′-snnnnNfNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 11S: 5′-snnnnnNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 12S: 5′-snnnnnNfNfNfdNnnnnnnnnnnsnsn-3′, wherein “Nf” is a 2′ fluoro-modified nucleoside, “dN” is a 2′ deoxy-modified nucleoside, “n” is a 2′ O-methyl modified nucleoside, and “s” is a phosphorothioate linkage. In some embodiments, the sense strand comprises modification pattern 13S: 5′-snnnnnNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 14S: 5′-snnnnNfnnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 15S: 5′-snnnnNfnnnNfnNfnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 16S: 5′-snnnnNfNfnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 17S: 5′-snnnnnnnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 18S: 5′-snnnnnNfNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 19S: 5′-snnnnNfnnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 20S: 5′-snnnnnNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 21S: 5′-snnnnnNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 22S: 5′-nsnsnnnnNfnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 23S: 5′-nsnsnnnNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 24S: 5′-nsnsnnnNfNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 25S: 5′-nsnsnnnNfNfnNfnNfNnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 26S: 5′-nsnsnnNfnnnNfnNfnnnnnnnnsnsn-3′, wherein “NF” is a 2′ fluoro-modified nucleoside, “n” is a 2′ O-methyl modified nucleoside, and “s” is a phosphorothioate linkage. In some embodiments, the sense strand comprises modification pattern 27S: 5′-nsnsnnNfnNfnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 28S: 5′-nsnsnnNfNfnnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 29S: 5′-nsnsnnnnnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 30S: 5′-nsnsnnnnnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 31S: 5′-nsnsnnnNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 32S: 5′-nsnsnnNfnnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 33S: 5′-nsnsnnNfNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 34S: 5′-nsnsnnnnnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 35S: 5′-nsnsnnnNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 36S: 5′-nsnsnnnNfnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 37S: 5′-nsnsnnnNfNfNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 38S: 5′-nsnsnnNfnnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 39S: 5′-nsnsnnNfNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 40S: 5′-nsnsnnNfNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 41S: 5′-nsnsnnNfNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 42S: 5′-nsnsnnnnNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 43S: 5′-nsnsnnnNfnnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 44S: 5′-nsnsnnnnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 45S: 5′-nsnsnnnnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 46S: 5′-nsnsnnnnNfNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 47S: 5′-nsnsnnnNfnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 48S: 5′-nsnsnnnNfNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 49S: 5′-nsnsnnNfnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 50S: 5′-nsnsnnnNfNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 51S: 5′-nsnsnnNfnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 52S: 5′-nsnsnnNfnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 53S:5′-snnnnnNfNfNfdNnnnnnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 54S:5′-snnnnnNfNfNfNfnnnnnNmnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 55S: 5′-snnnnTmNfNfNfNfnnnNmnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 56S:5′-snnnnnnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 57S:5′-snnnnnnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 58S:5′-snnnnNfndNnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 59S:5′-snnnNmnNfNfNfNfnnnNmnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 60S:5′-snnnNmnNfNfNfNfnnNmnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 61S:5′-snnnNmnNfNfNfNfnnnNmnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 62S:5′-snnnNmnNfNfNfNfnnnNmnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-0-(2-methoxyethyl) thymine. In some embodiments, the sense strand comprises modification pattern 63S:5′-snnnNmnNfNfNfNfnnnNmnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 64S:5′-snnnnNfnNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 65AS:5′-snnnNmnnNfNfNfNfnnNmnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 66AS:5′-snnnNmNfnNfNfNfNfnnNmnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 67AS:5′-snnnNmnNfNfNfNfnnNmnnnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 68AS:5′-snnnNmnNfNfNfNfnnnnNmnnnnnsnsn-3′, wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-0-(2-methoxyethyl) thymine. In some embodiments, the sense strand comprises modification pattern 69AS:5′-snnnNmNfnNfNfNfNfnNmnnnnnnnsnsn-3′ wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine. In some embodiments, the sense strand comprises modification pattern 70AS:5′-snnnNmNfnNfNfNfNfnnnNmnnnnnsnsn-3′ wherein “Nf” is a 2′ fluoro-modified nucleoside, “n” is a 2′ O-methyl modified nucleoside, “Nm” is a 2′-O-(2-methoxyethyl) nucleoside, and “s” is a phosphorothioate linkage. In some embodiments, the Nm is a 2′-O-(2-methoxyethyl) thymine.

In some embodiments, the composition comprises an oligonucleotide that inhibits the expression of GPAM, wherein the oligonucleotide comprises an siRNA comprising a sense strand and an antisense strand, wherein the antisense strand comprises modification pattern IAS: 5′-nsNfsnNfnNfnNfnNfnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: 5′-nsNfsnnnNfnNffnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: 5′-nsNfsnnnNfnnnnnnnNfnfnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: 5′-nsNfsnNfnNfnnnnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 5AS: 5′-nsNfsnnnnnnnnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 6AS: 5′-nsNfsnnnNfnnNfnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 7AS: 5′-nsNfsnNfnNfNfnNfnNfnNfNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 8AS: 5′-nsNfsnnnnnnnnnnnNfnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 9AS:5′-nsNfsnnnNfnNfnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 10AS: 5′-nsNfsnNfnNfnnNfinnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 11AS:5′-nsNfsnNfnNfnfnNfnNfnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 12AS:5′-nsNfsnNfnnNfnNfnNfnnNfnNfnNfnsnsn-3′ wherein “Nf” is a 2′ fluoro-modified nucleoside, “n” is a 2′ O-methyl modified nucleoside, and “s” is a phosphorothioate linkage. In some embodiments, the sense strand comprises modification pattern 13AS:5′-nsNfsnnNfnfNfnNfffnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 14AS:5′-nsNfsnNfnNfnNfnnnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 15AS:5′-nsNfsnnnNfnNfnnnNfnNfnNfnNfasnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 16AS:5′-nsNfsnnnNfnNfnNfnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 17AS:5′-nsNfsnNfnnNfnnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 18AS:5′-nsNfsnNfnnNfnnNfnnnNfnNfNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 19AS:5′-nsNfsnnNfnNfnnNfnNfNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 20AS:5′-nsNfsnNfnnnNfnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 21AS:5′-nsNfsnnnnNfnNfnnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 22AS:5′-nsNfsnNfnnNfnNfnnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 23AS:5′-nsNfsnnNfnNfnNfnnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 24AS:5′-nsNfsnnNfnNfnNfnnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 25AS:5′-nsNfsnNfnNfnNfnNfnNfnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 26AS:5′-nsNfsnNfnnNfnnnnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 27AS:5′-nsNfsnNfnnNfnNfnnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 28AS:5′-nsNfsnnNfnNfnnNfnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 29AS:5′-nsNfsnnnNfnnnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 30AS:5′-nsNfsnnnNfNfnnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 31AS:5′-nsNfsnnNfnNfNfnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 32AS:5′-nsNfsnnNfnNfnfnnnNfnNfnNfnNfnsnsn-3′, wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of GPAM, wherein the oligonucleotide comprises an siRNA comprising a sense strand and an antisense strand, wherein the sense strand comprises pattern 1S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 2S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 3S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 4S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 5S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 6S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 7S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 8S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 9S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 10S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 11S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 12S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 13S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 14S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 15S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 16S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 17S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 18S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 19S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 20S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 21S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 22S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 23S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 24S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 25S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 26S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 27S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 28S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 29S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 30S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 31S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 32S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 33S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 34S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 35S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 36S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 37S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 38S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 39S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 40S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 41S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 42S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 43S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 44S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 45S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 46S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 47S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 48S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 49S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 50S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 51S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 52S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 53S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 54S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 55S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 56S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 57S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 58S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 59S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 60S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 61S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 62S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 63S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 64S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 65S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 66S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 67S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 68S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 69S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the sense strand comprises pattern 70S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS.

In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 54S, 55S, 56S, 57S, 58S, 59S, 60S, 61S, 62S, 63S, 64S, 65S, 66S, 67S, 685, 69S, or 70S and the antisense strand comprises pattern 1AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 2AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 3AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 4AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 5AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 515, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 6AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 7AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 8AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 9AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 515, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 10AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 505, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 11AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 12AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 13AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 505, 51S, 52S, 52S, 54S, 55S, 56S, 57S, 58S, 59S, 60S, 615, 62S, 63S, 64S, 65S, 66S, 67S, 68S, 69S, or 70S and the antisense strand comprises pattern 14AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 15AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 16AS. In some embodiments, the sense strand comprises pattern 1S, 2S, 3S, 4S, 5S, 6S, 7S, 8S, 9S, 10S, 1 1S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 17AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 455, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 18AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 19AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 20AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 21AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 22AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 23AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 24AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 25AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 26AS. In some embodiments, the sense strand comprises pattern 1S, 2S, 3S, 4S, 5S, 6S, 7S, 8S, 9S, 10S, 1 1S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 27AS. In some embodiments, the sense strand comprises pattern 1S, 2S, 3S, 4S, 5S, 6S, 7S, 8S, 9S, 10S, 11S, 12S, 135, 14S, 15S, 165, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 28AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 29AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 30AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 31AS. In some embodiments, the sense strand comprises pattern 1S, 2S, 3S, 4S, 5S, 6S, 7S, 8S, 9S, 10S, S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S and the antisense strand comprises pattern 32AS.

In some embodiments, the sense strand comprises modification pattern 1S, 2S, 3S, 4S, 5S, 6S, 7S, 8S, 9S, 10S, 11S, 12S, 135, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, or 70S. In some embodiments, the sense strand comprises modification pattern 1AS, 2AS, 3AS, 4AS, 5AS, 6AS, 7AS, or 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the antisense strand comprises modification pattern 1AS, 2AS, 3AS, 4AS, 5AS, 6AS, 7AS, or 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS. In some embodiments, the antisense strand comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 585, 59S, 60S, 61S, 62S, 63S, 64S, 65S, 66S, 67S, 685, 69S, or 70S. In some embodiments, the sense strand or the antisense strand comprises modification pattern ASO1.

In some embodiments, purines of the sense strand comprise 2′ fluoro modified purines. In some embodiments, purines of the sense strand comprise 2′-O-methyl modified purines. In some embodiments, purines of the sense strand comprise a mixture of 2′ fluoro and 2′-O-methyl modified purines. In some embodiments, all purines of the sense strand comprise 2′ fluoro modified purines. In some embodiments, all purines of the sense strand comprise 2′-O-methyl modified purines. In some embodiments, all purines of the sense strand comprise a mixture of 2′ fluoro and 2′-O-methyl modified purines.

In some embodiments, pyrimidines of the sense strand comprise 2′ fluoro modified pyrimidines. In some embodiments, pyrimidines of the sense strand comprise 2′-O-methyl modified pyrimidines. In some embodiments, pyrimidines of the sense strand comprise a mixture of 2′ fluoro and 2′-O-methyl modified pyrimidines. In some embodiments, all pyrimidines of the sense strand comprise 2′ fluoro modified pyrimidines. In some embodiments, all pyrimidines of the sense strand comprise 2′-O-methyl modified pyrimidines. In some embodiments, all pyrimidines of the sense strand comprise a mixture of 2′ fluoro and 2′-O-methyl modified pyrimidines.

In some embodiments, purines of the sense strand comprise 2′ fluoro modified purines, and pyrimidines of the sense strand comprise a mixture of 2′ fluoro and 2′-O-methyl modified pyrimidines. In some embodiments, purines of the sense strand comprise 2′-O-methyl modified purines, and pyrimidines of the sense strand comprise a mixture of 2′ fluoro and 2′-O-methyl modified pyrimidines. In some embodiments, purines of the sense strand comprise 2′ fluoro modified purines, and pyrimidines of the sense strand comprise 2′-O-methyl modified pyrimidines. In some embodiments, purines of the sense strand comprise 2′-O-methyl modified purines, and pyrimidines of the sense strand comprise 2′ fluoro modified pyrimidines. In some embodiments, pyrimidines of the sense strand comprise 2′ fluoro modified pyrimidines, and purines of the sense strand comprise a mixture of 2′ fluoro and 2′-O-methyl modified purines. In some embodiments, pyrimidines of the sense strand comprise 2′-O-methyl modified pyrimidines, and purines of the sense strand comprise a mixture of 2′ fluoro and 2′-O-methyl modified purines. In some embodiments, pyrimidines of the sense strand comprise 2′ fluoro modified pyrimidines, and purines of the sense strand comprise 2′-O-methyl modified purines. In some embodiments, pyrimidines of the sense strand comprise 2′-O-methyl modified pyrimidines, and purines of the sense strand comprise 2′ fluoro modified purines.

In some embodiments, all purines of the sense strand comprise 2′ fluoro modified purines, and all pyrimidines of the sense strand comprise a mixture of 2′ fluoro and 2′-O-methyl modified pyrimidines. In some embodiments, all purines of the sense strand comprise 2′-O-methyl modified purines, and all pyrimidines of the sense strand comprise a mixture of 2′ fluoro and 2′-O-methyl modified pyrimidines. In some embodiments, all purines of the sense strand comprise 2′ fluoro modified purines, and all pyrimidines of the sense strand comprise 2′-O-methyl modified pyrimidines. In some embodiments, all purines of the sense strand comprise 2′-O-methyl modified purines, and all pyrimidines of the sense strand comprise 2′ fluoro modified pyrimidines. In some embodiments, all pyrimidines of the sense strand comprise 2′ fluoro modified pyrimidines, and all purines of the sense strand comprise a mixture of 2′ fluoro and 2′-O-methyl modified purines. In some embodiments, all pyrimidines of the sense strand comprise 2′-O-methyl modified pyrimidines, and all purines of the sense strand comprise a mixture of 2′ fluoro and 2′-O-methyl modified purines. In some embodiments, all pyrimidines of the sense strand comprise 2′ fluoro modified pyrimidines, and all purines of the sense strand comprise 2′-O-methyl modified purines. In some embodiments, all pyrimidines of the sense strand comprise 2′-O-methyl modified pyrimidines, and all purines of the sense strand comprise 2′ fluoro modified purines.

In some embodiments, purines of the antisense strand comprise 2′ fluoro modified purines. In some embodiments, purines of the antisense strand comprise 2′-O-methyl modified purines. In some embodiments, purines of the antisense strand comprise a mixture of 2′ fluoro and 2′-O-methyl modified purines. In some embodiments, all purines of the antisense strand comprise 2′ fluoro modified purines. In some embodiments, all purines of the antisense strand comprise 2′-O-methyl modified purines. In some embodiments, all purines of the antisense strand comprise a mixture of 2′ fluoro and 2′-O-methyl modified purines.

In some embodiments, pyrimidines of the antisense strand comprise 2′ fluoro modified pyrimidines. In some embodiments, pyrimidines of the antisense strand comprise 2′-O-methyl modified pyrimidines. In some embodiments, pyrimidines of the antisense strand comprise a mixture of 2′ fluoro and 2′-O-methyl modified pyrimidines. In some embodiments, all pyrimidines of the antisense strand comprise 2′ fluoro modified pyrimidines. In some embodiments, all pyrimidines of the antisense strand comprise 2′-O-methyl modified pyrimidines. In some embodiments, all pyrimidines of the antisense strand comprise a mixture of 2′ fluoro and 2′-O-methyl modified pyrimidines.

In some embodiments, purines of the antisense strand comprise 2′ fluoro modified purines, and pyrimidines of the antisense strand comprise a mixture of 2′ fluoro and 2′-O-methyl modified pyrimidines. In some embodiments, purines of the antisense strand comprise 2′-O-methyl modified purines, and pyrimidines of the antisense strand comprise a mixture of 2′ fluoro and 2′-O-methyl modified pyrimidines. In some embodiments, purines of the antisense strand comprise 2′ fluoro modified purines, and pyrimidines of the antisense strand comprise 2′-O-methyl modified pyrimidines. In some embodiments, purines of the antisense strand comprise 2′-O-methyl modified purines, and pyrimidines of the antisense strand comprise 2′ fluoro modified pyrimidines. In some embodiments, pyrimidines of the antisense strand comprise 2′ fluoro modified pyrimidines, and purines of the antisense strand comprise a mixture of 2′ fluoro and 2′-O-methyl modified purines. In some embodiments, pyrimidines of the antisense strand comprise 2′-O-methyl modified pyrimidines, and purines of the antisense strand comprise a mixture of 2′ fluoro and 2′-O-methyl modified purines. In some embodiments, pyrimidines of the antisense strand comprise 2′ fluoro modified pyrimidines, and purines of the antisense strand comprise 2′-O-methyl modified purines. In some embodiments, pyrimidines of the antisense strand comprise 2′-O-methyl modified pyrimidines, and purines of the antisense strand comprise 2′ fluoro modified purines.

In some embodiments, all purines of the antisense strand comprise 2′ fluoro modified purines, and all pyrimidines of the antisense strand comprise a mixture of 2′ fluoro and 2′-O-methyl modified pyrimidines. In some embodiments, all purines of the antisense strand comprise 2′-O-methyl modified purines, and all pyrimidines of the antisense strand comprise a mixture of 2′ fluoro and 2′-O-methyl modified pyrimidines. In some embodiments, all purines of the antisense strand comprise 2′ fluoro modified purines, and all pyrimidines of the antisense strand comprise 2′-O-methyl modified pyrimidines. In some embodiments, all purines of the antisense strand comprise 2′-O-methyl modified purines, and all pyrimidines of the antisense strand comprise 2′ fluoro modified pyrimidines. In some embodiments, all pyrimidines of the antisense strand comprise 2′ fluoro modified pyrimidines, and all purines of the antisense strand comprise a mixture of 2′ fluoro and 2′-O-methyl modified purines. In some embodiments, all pyrimidines of the antisense strand comprise 2′-O-methyl modified pyrimidines, and all purines of the antisense strand comprise a mixture of 2′ fluoro and 2′-O-methyl modified purines. In some embodiments, all pyrimidines of the antisense strand comprise 2′ fluoro modified pyrimidines, and all purines of the antisense strand comprise 2′-O-methyl modified purines. In some embodiments, all pyrimidines of the antisense strand comprise 2′-O-methyl modified pyrimidines, and all purines of the antisense strand comprise 2′ fluoro modified purines.

Disclosed herein, in some embodiments, are modified oligonucleotides. The modified oligonucleotide may be an siRNA that includes modifications to the ribose rings, and phosphate linkages. The modifications may be in particular patterns that maximize cell delivery, stability, and efficiency. The siRNA may also include a vinyl phosphonate and a hydrophobic group. These modifications may aid in delivery to a cell or tissue within a subject. The modified oligonucleotide may be used in a method such as a treatment method or a method of reducing gene expression.

In some embodiments, the oligonucleotide comprises a duplex consisting of 21 nucleotide single strands with base pairing between 19 of the base pairs. In some embodiments, the duplex comprises single-stranded 2 nucleotide overhangs are at the 3′ ends of each strand. One strand (antisense strand) is complementary to a GPAM mRNA. Each end of the antisense strand has one to two phosphorothioate bonds. The 5′ end has an optional phosphate mimic such as a vinyl phosphonate. In some embodiments, the oligonucleotide is used to knock down a GPAM mRNA or a target protein. In some embodiments, the sense strand has the same sequence as the GPAM mRNA. In some embodiments, there are 1-2 phosphorothioates at the 3′ end. In some embodiments, there are 1 or no phosphorothioates at the 5′ end. In some embodiments, there is a hydrophobic conjugate of 12 to 25 carbons attached at the 5′ end via a phosphodiester bond.

In some cases, the sense strand of any of the siRNAs comprises siRNA with a particular modification pattern. In some embodiments of the modification pattern, position 9 counting from the 5′ end of the sense strand may have a 2′F modification. In some embodiments, when position 9 of the sense strand is a pyrimidine, then all purines in the sense strand have a 2′OMe modification. In some embodiments, when position 9 is the only pyrimidine between positions 5 and 11 of the sense stand, then position 9 is the only position with a 2′F modification in the sense strand. In some embodiments, when position 9 and only one other base between positions 5 and 11 of the sense strand are pyrimidines, then both of these pyrimidines are the only two positions with a 2′F modification in the sense strand. In some embodiments, when position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of the sense strand, then all combinations of pyrimidines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some embodiments, when position 9 of the sense strand is a purine, then all purines in the sense strand have a 2′OMe modification. In some embodiments, when position 9 is the only purine between positions 5 and 11 of the sense stand, then position 9 is the only position with a 2′F modification in the sense strand. In some embodiments, when position 9 and only one other base between positions 5 and 11 of the sense strand are purines, then both of these purines are the only two positions with a 2′F modification in the sense strand. In some embodiments, when position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of the sense strand, then all combinations of purines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some embodiments, the siRNA comprises a sense strand, an antisense strand, and a lipid moiety connected to an end of the sense or antisense strand; wherein the lipid moiety comprises a phenyl or cyclohexanyl linker, wherein the linker is connected to a lipid and to the end of the sense or antisense strand. In some embodiments, any one of the following is true with regard to the sense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′ fluoro modified purines, and all pyrimidines comprise 2′-O-methyl modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′ fluoro modified pyrimidines, and all purines comprise 2′-O-methyl modified purines. In some embodiments, any one of the following is true with regard to the antisense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′-O-methyl modified purines, and all pyrimidines comprise 2′ fluoro modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′-O-methyl modified pyrimidines, and all purines comprise 2′ fluoro modified purines. In some embodiments, the siRNA comprises comprising a sense strand and an antisense strand; wherein the antisense strand comprises a 5′ end comprising a vinyl phosphonate and 2 phosphorothioate linkages, and a 3′ end comprising 2 phosphorothioate linkages; wherein the sense strand comprises a 5′ end comprising a hydrophobic moiety, and a 3′ end comprising 2 phosphorothioate linkages; wherein any one of the following is true with regard to the sense strand: all purines comprise 2′ fluoro modified purines, and all pyrimidines comprise a mixture of 2′ fluoro and 2′-O-methyl modified pyrimidines, all purines comprise 2′-O-methyl modified purines, and all pyrimidines comprise a mixture of 2′ fluoro and 2′-O-methyl modified pyrimidines, all purines comprise 2′ fluoro modified purines, and all pyrimidines comprise 2′-O-methyl modified pyrimidines, all pyrimidines comprise 2′ fluoro modified pyrimidines, and all purines comprise a mixture of 2′ fluoro and 2′-O-methyl modified purines, all pyrimidines comprise 2′-O-methyl modified pyrimidines, and all purines comprise a mixture of 2′ fluoro and 2′-O-methyl modified purines, or all pyrimidines comprise 2′ fluoro modified pyrimidines, and all purines comprise 2′-O-methyl modified purines; and wherein any one of the following is true with regard to the antisense strand: all purines comprise 2′ fluoro modified purines, and all pyrimidines comprise a mixture of 2′ fluoro and 2′-O-methyl modified pyrimidines, all purines comprise 2′-O-methyl modified purines, and all pyrimidines comprise a mixture of 2′ fluoro and 2′-O-methyl modified pyrimidines, all purines comprise 2′-O-methyl modified purines, and all pyrimidines comprise 2′ fluoro modified pyrimidines, all pyrimidines comprise 2′ fluoro modified pyrimidines, and all purines comprise a mixture of 2′ fluoro and 2′-O-methyl modified purines, all pyrimidines comprise 2′-O-methyl modified pyrimidines, and all purines comprise a mixture of 2′ fluoro and 2′-O-methyl modified purines, or all pyrimidines comprise 2′-O-methyl modified pyrimidines, and all purines comprise 2′ fluoro modified purines.

In some embodiments, any one of the following is true with regard to the sense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′ fluoro modified purines, and all pyrimidines comprise 2′-O-methyl modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′ fluoro modified pyrimidines, and all purines comprise 2′-O-methyl modified purines; with the proviso that in any of the foregoing, the sense strand may include a deoxy nucleoside. In some embodiments, all purines comprise 2′ fluoro modified purines, and all pyrimidines comprise a mixture of 2′ fluoro and 2′-O-methyl modified pyrimidines; with the proviso that a deoxy nucleoside may be included in the sense strand. In some embodiments, in the sense strand, all purines comprise 2′-O-methyl modified purines, and all pyrimidines comprise a mixture of 2′ fluoro and 2′-O-methyl modified pyrimidines; with the proviso that a deoxy nucleoside may be included in the sense strand. In some embodiments, in the sense strand, all purines comprise 2′ fluoro modified purines, and all pyrimidines comprise 2′-O-methyl modified pyrimidines; with the proviso that a deoxy nucleoside may be included in the sense strand. In some embodiments, all pyrimidines comprise 2′ fluoro modified pyrimidines, and all purines comprise a mixture of 2′ fluoro and 2′-O-methyl modified purines; with the proviso that a deoxy nucleoside may be included in the sense strand. In some embodiments, in the sense strand, all pyrimidines comprise 2′-O-methyl modified pyrimidines, and all purines comprise a mixture of 2′ fluoro and 2′-O-methyl modified purines; with the proviso that a deoxy nucleoside may be included in the sense strand. In some embodiments, in the sense strand, all pyrimidines comprise 2′ fluoro modified pyrimidines, and all purines comprise 2′-O-methyl modified purines; with the proviso that a deoxy nucleoside may be included in the sense strand. In some embodiments, the sense strand includes the deoxy nucleoside. The deoxy nucleoside may be at nucleoside position 9 of the sense strand. In some embodiments, the sense strand does not include a deoxy nucleoside. The deoxy nucleoside of the sense strand may be otherwise unmodified.

In some embodiments, any one of the following is true with regard to the antisense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′-O-methyl modified purines, and all pyrimidines comprise 2′ fluoro modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′-O-methyl modified pyrimidines, and all purines comprise 2′ fluoro modified purines; with the proviso that in any of the foregoing, the sense strand may include a deoxy nucleoside. In some embodiments, all purines comprise 2′ fluoro modified purines, and all pyrimidines comprise a mixture of 2′ fluoro and 2′-O-methyl modified pyrimidines; with the proviso that a deoxy nucleoside may be included in the antisense strand. In some embodiments, in the antisense strand, all purines comprise 2′-O-methyl modified purines, and all pyrimidines comprise a mixture of 2′ fluoro and 2′-O-methyl modified pyrimidines; with the proviso that a deoxy nucleoside may be included in the antisense strand. In some embodiments, in the antisense strand, all purines comprise 2′-O-methyl modified purines, and all pyrimidines comprise 2′ fluoro modified pyrimidines; with the proviso that a deoxy nucleoside may be included in the antisense strand. In some embodiments, in the antisense strand, all pyrimidines comprise 2′ fluoro modified pyrimidines, and all purines comprise a mixture of 2′ fluoro and 2′-O-methyl modified purines; with the proviso that a deoxy nucleoside may be included in the antisense strand. In some embodiments, in the antisense strand, all pyrimidines comprise 2′-O-methyl modified pyrimidines, and all purines comprise a mixture of 2′ fluoro and 2′-O-methyl modified purines; with the proviso that a deoxy nucleoside may be included in the antisense strand. In some embodiments, in the antisense strand, all pyrimidines comprise 2′-O-methyl modified pyrimidines, and all purines comprise 2′ fluoro modified purines; with the proviso that a deoxy nucleoside may be included in the antisense strand. In some embodiments, the antisense strand includes the deoxy nucleoside. The deoxy nucleoside may be at nucleoside position 9 of the antisense strand. In some embodiments, the antisense strand does not include a deoxy nucleoside. The deoxy nucleoside of the antisense strand may be otherwise unmodified.

In some embodiments, any one of the following is true with regard to the sense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′ fluoro modified purines, and all pyrimidines comprise 2′-O-methyl modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′ fluoro modified pyrimidines, and all purines comprise 2′-O-methyl modified purines; with the proviso that in any of the foregoing, the sense strand may include a deoxy nucleoside or a 2′-O-(2-methoxyethyl) nucleoside. In some embodiments, all purines comprise 2′ fluoro modified purines, and all pyrimidines comprise a mixture of 2′ fluoro and 2′-O-methyl modified pyrimidines; with the proviso that a deoxy nucleoside or a 2′-O-(2-methoxyethyl) nucleoside may be included in the sense strand. In some embodiments, in the sense strand, all purines comprise 2′-O-methyl modified purines, and all pyrimidines comprise a mixture of 2′ fluoro and 2′-O-methyl modified pyrimidines; with the proviso that a deoxy nucleoside or a 2′-O-(2-methoxyethyl) nucleoside may be included in the sense strand. In some embodiments, in the sense strand, all purines comprise 2′ fluoro modified purines, and all pyrimidines comprise 2′-O-methyl modified pyrimidines; with the proviso that a deoxy nucleoside or a 2′-O-(2-methoxyethyl) nucleoside may be included in the sense strand. In some embodiments, all pyrimidines comprise 2′ fluoro modified pyrimidines, and all purines comprise a mixture of 2′ fluoro and 2′-O-methyl modified purines; with the proviso that a deoxy nucleoside or a 2′-O-(2-methoxyethyl) nucleoside may be included in the sense strand. In some embodiments, in the sense strand, all pyrimidines comprise 2′-O-methyl modified pyrimidines, and all purines comprise a mixture of 2′ fluoro and 2′-O-methyl modified purines; with the proviso that a deoxy nucleoside or a 2′-O-(2-methoxyethyl) nucleoside may be included in the sense strand. In some embodiments, in the sense strand, all pyrimidines comprise 2′ fluoro modified pyrimidines, and all purines comprise 2′-O-methyl modified purines; with the proviso that a deoxy nucleoside or a 2′-O-(2-methoxyethyl) nucleoside may be included in the sense strand. In some embodiments, the sense strand includes the deoxy nucleoside. The deoxy nucleoside may be at nucleoside position 9 of the sense strand. In some embodiments, the sense strand does not include a deoxy nucleoside. The deoxy nucleoside of the sense strand may be otherwise unmodified. In some embodiments, the sense strand includes the a 2′-O-(2-methoxyethyl) nucleoside. The 2′-O-(2-methoxyethyl) nucleoside may be at nucleoside position 4 of the sense strand. The 2′-O-(2-methoxyethyl) nucleoside may include a 2′-O-(2-methoxyethyl) thymine nucleoside. In some embodiments, the sense strand does not include the a 2′-O-(2-methoxyethyl) nucleoside. The 2′-O-(2-methoxyethyl) nucleoside of the sense strand may be otherwise unmodified.

In some embodiments, any one of the following is true with regard to the antisense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′-O-methyl modified purines, and all pyrimidines comprise 2′ fluoro modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′-O-methyl modified pyrimidines, and all purines comprise 2′ fluoro modified purines; with the proviso that in any of the foregoing, the sense strand may include a deoxy nucleoside or a 2′-O-(2-methoxyethyl) nucleoside. In some embodiments, all purines comprise 2′ fluoro modified purines, and all pyrimidines comprise a mixture of 2′ fluoro and 2′-O-methyl modified pyrimidines; with the proviso that a deoxy nucleoside or a 2′-O-(2-methoxyethyl) nucleoside may be included in the antisense strand. In some embodiments, in the antisense strand, all purines comprise 2′-O-methyl modified purines, and all pyrimidines comprise a mixture of 2′ fluoro and 2′-O-methyl modified pyrimidines; with the proviso that a deoxy nucleoside or a 2′-O-(2-methoxyethyl) nucleoside may be included in the antisense strand. In some embodiments, in the antisense strand, all purines comprise 2′-O-methyl modified purines, and all pyrimidines comprise 2′ fluoro modified pyrimidines; with the proviso that a deoxy nucleoside or a 2′-O-(2-methoxyethyl) nucleoside may be included in the antisense strand. In some embodiments, in the antisense strand, all pyrimidines comprise 2′ fluoro modified pyrimidines, and all purines comprise a mixture of 2′ fluoro and 2′-O-methyl modified purines; with the proviso that a deoxy nucleoside or a 2′-O-(2-methoxyethyl) nucleoside may be included in the antisense strand. In some embodiments, in the antisense strand, all pyrimidines comprise 2′-O-methyl modified pyrimidines, and all purines comprise a mixture of 2′ fluoro and 2′-O-methyl modified purines; with the proviso that a deoxy nucleoside or a 2′-O-(2-methoxyethyl) nucleoside may be included in the antisense strand. In some embodiments, in the antisense strand, all pyrimidines comprise 2′-O-methyl modified pyrimidines, and all purines comprise 2′ fluoro modified purines; with the proviso that a deoxy nucleoside or a 2′-O-(2-methoxyethyl) nucleoside may be included in the antisense strand. In some embodiments, the antisense strand includes the deoxy nucleoside. The deoxy nucleoside may be at nucleoside position 9 of the antisense strand. In some embodiments, the antisense strand does not include a deoxy nucleoside. The deoxy nucleoside of the antisense strand may be otherwise unmodified. In some embodiments, the antisense strand includes the a 2′-O-(2-methoxyethyl) nucleoside. The 2′-O-(2-methoxyethyl) nucleoside may be at nucleoside position 4 of the sense strand. The 2′-O-(2-methoxyethyl) nucleoside may include a 2′-O-(2-methoxyethyl) thymine nucleoside. In some embodiments, the antisense strand does not include the a 2′-O-(2-methoxyethyl) nucleoside. The 2′-O-(2-methoxyethyl) nucleoside of the antisense strand may be otherwise unmodified.

In some embodiments, any one of the following is true with regard to the sense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′ fluoro modified purines, and all pyrimidines comprise 2′-O-methyl modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′ fluoro modified pyrimidines, and all purines comprise 2′-O-methyl modified purines; with the proviso that in any of the foregoing, the sense strand may include a 2′-O-(2-methoxyethyl) nucleoside. In some embodiments, all purines comprise 2′ fluoro modified purines, and all pyrimidines comprise a mixture of 2′ fluoro and 2′-O-methyl modified pyrimidines; with the proviso that a 2′-O-(2-methoxyethyl) nucleoside may be included in the sense strand. In some embodiments, in the sense strand, all purines comprise 2′-O-methyl modified purines, and all pyrimidines comprise a mixture of 2′ fluoro and 2′-O-methyl modified pyrimidines; with the proviso that a 2′-O-(2-methoxyethyl) nucleoside may be included in the sense strand. In some embodiments, in the sense strand, all purines comprise 2′ fluoro modified purines, and all pyrimidines comprise 2′-O-methyl modified pyrimidines; with the proviso that a 2′-O-(2-methoxyethyl) nucleoside may be included in the sense strand. In some embodiments, all pyrimidines comprise 2′ fluoro modified pyrimidines, and all purines comprise a mixture of 2′ fluoro and 2′-O-methyl modified purines; with the proviso that a 2′-O-(2-methoxyethyl) nucleoside may be included in the sense strand. In some embodiments, in the sense strand, all pyrimidines comprise 2′-O-methyl modified pyrimidines, and all purines comprise a mixture of 2′ fluoro and 2′-O-methyl modified purines; with the proviso that a 2′-O-(2-methoxyethyl) nucleoside may be included in the sense strand. In some embodiments, in the sense strand, all pyrimidines comprise 2′ fluoro modified pyrimidines, and all purines comprise 2′-O-methyl modified purines; with the proviso that a 2′-O-(2-methoxyethyl) nucleoside may be included in the sense strand. In some embodiments, the sense strand includes the a 2′-O-(2-methoxyethyl) nucleoside. The 2′-0-(2-methoxyethyl) nucleoside may be at nucleoside position 4 of the sense strand. The 2′-O-(2-methoxyethyl) nucleoside may include a 2′-O-(2-methoxyethyl) thymine nucleoside. In some embodiments, the sense strand does not include the a 2′-O-(2-methoxyethyl) nucleoside. The 2′-O-(2-methoxyethyl) nucleoside of the sense strand may be otherwise unmodified.

In some embodiments, any one of the following is true with regard to the antisense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′-O-methyl modified purines, and all pyrimidines comprise 2′ fluoro modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′-O-methyl modified pyrimidines, and all purines comprise 2′ fluoro modified purines; with the proviso that in any of the foregoing, the sense strand may include a deoxy nucleoside or a 2′-O-(2-methoxyethyl) nucleoside. In some embodiments, all purines comprise 2′ fluoro modified purines, and all pyrimidines comprise a mixture of 2′ fluoro and 2′-O-methyl modified pyrimidines; with the proviso that a 2′-O-(2-methoxyethyl) nucleoside may be included in the antisense strand. In some embodiments, in the antisense strand, all purines comprise 2′-O-methyl modified purines, and all pyrimidines comprise a mixture of 2′ fluoro and 2′-O-methyl modified pyrimidines; with the proviso that a 2′-O-(2-methoxyethyl) nucleoside may be included in the antisense strand. In some embodiments, in the antisense strand, all purines comprise 2′-O-methyl modified purines, and all pyrimidines comprise 2′ fluoro modified pyrimidines; with the proviso that a 2′-O-(2-methoxyethyl) nucleoside may be included in the antisense strand. In some embodiments, in the antisense strand, all pyrimidines comprise 2′ fluoro modified pyrimidines, and all purines comprise a mixture of 2′ fluoro and 2′-O-methyl modified purines; with the proviso that a 2′-0-(2-methoxyethyl) nucleoside may be included in the antisense strand. In some embodiments, in the antisense strand, all pyrimidines comprise 2′-O-methyl modified pyrimidines, and all purines comprise a mixture of 2′ fluoro and 2′-O-methyl modified purines; with the proviso that a 2′-O-(2-methoxyethyl) nucleoside may be included in the antisense strand. In some embodiments, in the antisense strand, all pyrimidines comprise 2′-O-methyl modified pyrimidines, and all purines comprise 2′ fluoro modified purines; with the proviso that a 2′-O-(2-methoxyethyl) nucleoside may be included in the antisense strand. In some embodiments, the antisense strand includes the a 2′-O-(2-methoxyethyl) nucleoside. The 2′-O-(2-methoxyethyl) nucleoside may be at nucleoside position 4 of the sense strand. The 2′-O-(2-methoxyethyl) nucleoside may include a 2′-O-(2-methoxyethyl) thymine nucleoside. In some embodiments, the antisense strand does not include the a 2′-O-(2-methoxyethyl) nucleoside. The 2′-O-(2-methoxyethyl) nucleoside of the antisense strand may be otherwise unmodified.

In some embodiments, any one of the following is true with regard to the sense strand, with the proviso that the sense strand may include a 2′ deoxy nucleoside: all purine nucleosides comprise 2′ fluoro, and all pyrimidine nucleosides are modified with a mixture of 2′ fluoro and 2′-O-methyl, all purine nucleosides comprise 2′-O-methyl, and all pyrimidine nucleosides are modified with a mixture of 2′ fluoro and 2′-O-methyl, all purine nucleosides comprise 2′ fluoro, and all pyrimidine nucleosides comprise 2′-O-methyl, all pyrimidine nucleosides comprise 2′ fluoro, and all purine nucleosides are modified with a mixture of 2′ fluoro and 2′-O-methyl, all pyrimidine nucleosides comprise 2′-O-methyl, and all purine nucleosides are modified with a mixture of 2′ fluoro and 2′-O-methyl, or all pyrimidine nucleosides comprise 2′ fluoro, and all purine nucleosides comprise 2′-O-methyl. In some embodiments, in the sense strand, all purine nucleosides comprise 2′ fluoro, and all pyrimidine nucleosides are modified with a mixture of 2′ fluoro and 2′-O-methyl, with the proviso that the sense strand may include a 2′ deoxy nucleoside. In some embodiments, in the sense strand, all purine nucleosides comprise 2′-O-methyl, and all pyrimidine nucleosides are modified with a mixture of 2′ fluoro and 2′-O-methyl, with the proviso that the sense strand may include a 2′ deoxy nucleoside. In some embodiments, in the sense strand, all purine nucleosides comprise 2′ fluoro, and all pyrimidine nucleosides comprise 2′-O-methyl, with the proviso that the sense strand may include a 2′ deoxy nucleoside. In some embodiments, in the sense strand, all pyrimidine nucleosides comprise 2′ fluoro, and all purine nucleosides are modified with a mixture of 2′ fluoro and 2′-O-methyl, with the proviso that the sense strand may include a 2′ deoxy nucleoside. In some embodiments, in the sense strand, all pyrimidine nucleosides comprise 2′-O-methyl, and all purine nucleosides are modified with a mixture of 2′ fluoro and 2′-O-methyl, with the proviso that the sense strand may include a 2′ deoxy nucleoside. In some embodiments, in the sense strand, all pyrimidine nucleosides comprise 2′ fluoro, and all purine nucleosides comprise 2′-O-methyl, with the proviso that the sense strand may include a 2′ deoxy nucleoside. In some embodiments, the sense strand includes the 2′ deoxy nucleoside. In some embodiments, the sense strand does not include the 2′ deoxy nucleoside. Some embodiments include a proviso that the sense strand may include a 2′-O-(2-methoxyethyl) nucleoside (e.g. at position 4, counting from 5′ to 3′). Some embodiments include the 2′-O-(2-methoxyethyl) nucleoside in the sense strand. Some embodiments do not include the 2′-O-(2-methoxyethyl) nucleoside in the sense strand.

In some embodiments, any one of the following is true with regard to the sense strand: all purine nucleosides comprise 2′ fluoro, and all pyrimidine nucleosides are modified with a mixture of 2′ fluoro and 2′-O-methyl, all purine nucleosides comprise 2′-O-methyl, and all pyrimidine nucleosides are modified with a mixture of 2′ fluoro and 2′-O-methyl, all purine nucleosides comprise 2′ fluoro, and all pyrimidine nucleosides comprise 2′-O-methyl, all pyrimidine nucleosides comprise 2′ fluoro, and all purine nucleosides are modified with a mixture of 2′ fluoro and 2′-O-methyl, all pyrimidine nucleosides comprise 2′-O-methyl, and all purine nucleosides are modified with a mixture of 2′ fluoro and 2′-O-methyl, or all pyrimidine nucleosides comprise 2′ fluoro, and all purine nucleosides comprise 2′-O-methyl. In some embodiments, in the sense strand, all purine nucleosides comprise 2′ fluoro, and all pyrimidine nucleosides are modified with a mixture of 2′ fluoro and 2′-O-methyl. In some embodiments, in the sense strand, all purine nucleosides comprise 2′-O-methyl, and all pyrimidine nucleosides are modified with a mixture of 2′ fluoro and 2′-O-methyl. In some embodiments, in the sense strand, all purine nucleosides comprise 2′ fluoro, and all pyrimidine nucleosides comprise 2′-O-methyl. In some embodiments, in the sense strand, all pyrimidine nucleosides comprise 2′ fluoro, and all purine nucleosides are modified with a mixture of 2′ fluoro and 2′-O-methyl. In some embodiments, in the sense strand, all pyrimidine nucleosides comprise 2′-O-methyl, and all purine nucleosides are modified with a mixture of 2′ fluoro and 2′-O-methyl. In some embodiments, in the sense strand, all pyrimidine nucleosides comprise 2′ fluoro, and all purine nucleosides comprise 2′-O-methyl. Some embodiments include a proviso that the sense strand may include a 2′-O-methoxyethyl nucleoside (e.g. at position 4, counting from 5′ to 3′). Some embodiments include the 2′-O-methoxyethyl nucleoside in the sense strand. Some embodiments do not include the 2′-O-methoxyethyl nucleoside in the sense strand.

In some embodiments, any one of the following is true with regard to the antisense strand, with the proviso that the antisense strand may include a 2′ deoxy nucleoside: all purine nucleosides comprise 2′ fluoro, and all pyrimidine nucleosides are modified with a mixture of 2′ fluoro and 2′-O-methyl, all purine nucleosides comprise 2′-O-methyl, and all pyrimidine nucleosides are modified with a mixture of 2′ fluoro and 2′-O-methyl, all purine nucleosides comprise 2′-O-methyl, and all pyrimidine nucleosides comprise 2′ fluoro, all pyrimidine nucleosides comprise 2′ fluoro, and all purine nucleosides are modified with a mixture of 2′ fluoro and 2′-O-methyl, all pyrimidine nucleosides comprise 2′-O-methyl, and all purine nucleosides are modified with a mixture of 2′ fluoro and 2′-O-methyl, or all pyrimidine nucleosides comprise 2′-O-methyl, and all purine nucleosides comprise 2′ fluoro. In some embodiments, in the antisense strand, all purine nucleosides comprise 2′ fluoro, and all pyrimidine nucleosides are modified with a mixture of 2′ fluoro and 2′-O-methyl, with the proviso that the antisense strand may include a 2′ deoxy nucleoside. In some embodiments, in the antisense strand, all purine nucleosides comprise 2′-O-methyl, and all pyrimidine nucleosides are modified with a mixture of 2′ fluoro and 2′-O-methyl, with the proviso that the antisense strand may include a 2′ deoxy nucleoside. In some embodiments, in the antisense strand, all purine nucleosides comprise 2′ fluoro, and all pyrimidine nucleosides comprise 2′-O-methyl, with the proviso that the antisense strand may include a 2′ deoxy nucleoside. In some embodiments, in the antisense strand, all pyrimidine nucleosides comprise 2′ fluoro, and all purine nucleosides are modified with a mixture of 2′ fluoro and 2′-O-methyl, with the proviso that the antisense strand may include a 2′ deoxy nucleoside. In some embodiments, in the antisense strand, all pyrimidine nucleosides comprise 2′-O-methyl, and all purine nucleosides are modified with a mixture of 2′ fluoro and 2′-O-methyl, with the proviso that the antisense strand may include a 2′ deoxy nucleoside. In some embodiments, in the antisense strand, all pyrimidine nucleosides comprise 2′ fluoro, and all purine nucleosides comprise 2′-O-methyl, with the proviso that the antisense strand may include a 2′ deoxy nucleoside. In some embodiments, the antisense strand includes the 2′ deoxy nucleoside. In some embodiments, the antisense strand does not include the 2′ deoxy nucleoside. Some embodiments include a proviso that the antisense strand may include a 2′-O-methoxyethyl nucleoside (e.g. at position 4, counting from 5′ to 3′). Some embodiments include the 2′-O-methoxyethyl nucleoside in the antisense strand. Some embodiments do not include the 2′-O-methoxyethyl nucleoside in the antisense strand.

In some embodiments, any one of the following is true with regard to the antisense strand: all purine nucleosides comprise 2′ fluoro, and all pyrimidine nucleosides are modified with a mixture of 2′ fluoro and 2′-O-methyl, all purine nucleosides comprise 2′-O-methyl, and all pyrimidine nucleosides are modified with a mixture of 2′ fluoro and 2′-O-methyl, all purine nucleosides comprise 2′-O-methyl, and all pyrimidine nucleosides comprise 2′ fluoro, all pyrimidine nucleosides comprise 2′ fluoro, and all purine nucleosides are modified with a mixture of 2′ fluoro and 2′-O-methyl, all pyrimidine nucleosides comprise 2′-O-methyl, and all purine nucleosides are modified with a mixture of 2′ fluoro and 2′-O-methyl, or all pyrimidine nucleosides comprise 2′-O-methyl, and all purine nucleosides comprise 2′ fluoro. In some embodiments, in the antisense strand, all purine nucleosides comprise 2′ fluoro, and all pyrimidine nucleosides are modified with a mixture of 2′ fluoro and 2′-O-methyl. In some embodiments, in the antisense strand, all purine nucleosides comprise 2′-O-methyl, and all pyrimidine nucleosides are modified with a mixture of 2′ fluoro and 2′-O-methyl. In some embodiments, in the antisense strand, all purine nucleosides comprise 2′ fluoro, and all pyrimidine nucleosides comprise 2′-O-methyl. In some embodiments, in the antisense strand, all pyrimidine nucleosides comprise 2′ fluoro, and all purine nucleosides are modified with a mixture of 2′ fluoro and 2′-O-methyl. In some embodiments, in the antisense strand, all pyrimidine nucleosides comprise 2′-O-methyl, and all purine nucleosides are modified with a mixture of 2′ fluoro and 2′-O-methyl. In some embodiments, in the antisense strand, all pyrimidine nucleosides comprise 2′ fluoro, and all purine nucleosides comprise 2′-O-methyl. Some embodiments include a proviso that the antisense strand may include a 2′-O-methoxyethyl nucleoside (e.g. at position 4, counting from 5′ to 3′). Some embodiments include the 2′-O-methoxyethyl nucleoside in the antisense strand. Some embodiments do not include the 2′-O-methoxyethyl nucleoside in the antisense strand.

In some embodiments, the antisense strand comprises one or two 3′ phosphorothioate linkages. For example, there may be a phosphorothioate linkage between the first and second nucleotides from the 3′ end of the antisense strand, or there may be phosphorothioate linkages between the first, second and third nucleotides from the 3′ end of the antisense strand. In some embodiments, the sense strand comprises one or two 5′ phosphorothioate linkages. For example, there may be a phosphorothioate linkage between the first and second nucleotides from the 5′ end of the sense strand, or there may be phosphorothioate linkages between the first, second and third nucleotides from the 5′ end of the sense strand. In some embodiments, the sense strand does not comprise one or two 5′ phosphorothioate linkages. For example, in some embodiments, there are no phosphorothioate linkages between the last 3 nucleotides at the 5′ end of the sense strand. In some embodiments, the sense strand comprises 5′ phosphate linkages. In some embodiments, the sense strand comprises one or two 3′ phosphorothioate linkages. For example, there may be a phosphorothioate linkage between the first and second nucleotides from the 3′ end of the sense strand, or there may be phosphorothioate linkages between the first, second and third nucleotides from the 3′ end of the sense strand.

In some embodiments, the antisense strand comprises a 5′ end comprising 2 phosphorothioate linkages. The 5′ end may comprise 3 nucleosides separated by the 2 phosphorothioate linkages. In some embodiments, the antisense strand comprises a 3′ end comprising 2 phosphorothioate linkages. The 3′ end may comprise 3 nucleosides separated by the 2 phosphorothioate linkages.

In some cases, position 9 of the sense strand can be a 2′deoxy. In these cases, 2′F and 2′OMe modifications may occur at the other positions of the sense strand. In some cases, the sense strand of any of the siRNAs comprises a modification pattern which conforms to these sense strand rules.

In some cases, the sense strand of any of the siRNAs comprises a modification pattern which conforms to these sense strand rules.

Disclosed herein, in some embodiments are compositions comprising an oligonucleotide that targets GPAM and when administered to a cell decreases expression of GPAM, wherein the oligonucleotide comprises a small interfering RNA (siRNA) comprising a sense strand and an antisense strand, wherein the sense strand comprises a sense strand sequence described herein in which at least one internucleoside linkage is modified and at least one nucleoside is modified, or an sense strand sequence comprising 1 or 2 nucleoside substitutions, additions, or deletions of the oligonucleotide sequence in which at least one internucleoside linkage is modified and at least one nucleoside is modified, and wherein the antisense strand comprises an antisense strand sequence described herein in which at least one internucleoside linkage is modified and at least one nucleoside is modified, or an oligonucleotide sequence comprising 1 or 2 nucleoside substitutions, additions, or deletions of the antisense strand sequence in which at least one internucleoside linkage is modified and at least one nucleoside is modified. Some embodiments relate to methods that include administering the composition to a subject.

In some embodiments, the siRNA comprises a sense strand having a sequence in accordance with any of SEQ ID NOs: 12709-12733. In some embodiments, the sense strand sequence comprises or consists of sequence at least 75% identical to any one of SEQ ID NOs: 12709-12733, at least 80% identical to any one of SEQ ID NOs: 12709-12733, at least 85% identical to of any one of SEQ ID NOs: 12709-12733, at least 90% identical to any one of SEQ ID NOs: 12709-12733, or at least 95% identical to any one of SEQ ID NOs: 12709-12733. In some embodiments, the sense strand sequence comprises or consists of the sequence of any one of SEQ ID NOs: 12709-12733, or a sense strand sequence thereof having 1, 2, 3, or 4 nucleoside substitutions, additions, or deletions. In some embodiments, the sense strand sequence comprises or consists of the sequence of any one of SEQ ID NOs: 12709-12733, or a sense strand sequence thereof having 1 or 2 nucleoside substitutions, additions, or deletions. In some embodiments, the sense strand sequence comprises or consists of a sequence 100% identical to SEQ ID NOs: 12709-12733. In some embodiments, the siRNA comprises a sense strand having a sequence in accordance with any of SEQ ID NOs: 12759-12866. In some embodiments, the sense strand sequence comprises or consists of sequence at least 75% identical to any one of SEQ ID NOs: 12759-12866, at least 80% identical to any one of SEQ ID NOs: 12759-12866, at least 85% identical to of any one of SEQ ID NOs: 12759-12866, at least 90% identical to any one of SEQ ID NOs: 12759-12866, or at least 95% identical to any one of SEQ ID NOs: 12759-12866. In some embodiments, the sense strand sequence comprises or consists of the sequence of any one of SEQ ID NOs: 12759-12866, or a sense strand sequence thereof having 1, 2, 3, or 4 nucleoside substitutions, additions, or deletions. In some embodiments, the sense strand sequence comprises or consists of the sequence of any one of SEQ ID NOs: 12759-12866, or a sense strand sequence thereof having 1 or 2 nucleoside substitutions, additions, or deletions. In some embodiments, the sense strand sequence comprises or consists of a sequence 100% identical to SEQ ID NOs: 12759-12866. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the first 19 nucleotides of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise an overhang. The sense strand may comprise a modification pattern described herein, such as a different set of modifications or modification pattern than the aforementioned sequences. The sense strand may comprise a lipid moiety. The sense strand may comprise a GalNAc moiety. In some embodiments, the siRNA comprises an antisense strand having a sequence in accordance with any of SEQ ID NOs: 12734-12758. In some embodiments, the antisense strand sequence comprises or consists of sequence at least 75% identical to any one of SEQ ID NOs: 12734-12758, at least 80% identical to any one of SEQ ID NOs: 12734-12758, at least 85% identical to of any one of SEQ ID NOs: 12734-12758, at least 90% identical to any one of SEQ ID NOs: 12734-12758, or at least 95% identical to any one of SEQ ID NOs: 12734-12758. In some embodiments, the antisense strand sequence comprises or consists of the sequence of any one of SEQ ID NOs: 12734-12758, or an antisense strand sequence thereof having 1, 2, 3, or 4 nucleoside substitutions, additions, or deletions. In some embodiments, the antisense strand sequence comprises or consists of the sequence of any one of SEQ ID NOs: 12734-12758, or an antisense strand sequence thereof having 1 or 2 nucleoside substitutions, additions, or deletions. In some embodiments, the antisense strand sequence comprises or consists of a sequence 100% identical to SEQ ID NOs: 12734-12758. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the first 19 nucleotides of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein, such as a different set of modifications or modification pattern than the aforementioned sequences. The antisense strand may comprise a lipid moiety or a GalNAc moiety.

In some embodiments, the siRNA comprises a sense strand having a sequence in accordance with any of SEQ ID NOs: 12868-12974. In some embodiments, the sense strand sequence comprises or consists of sequence at least 75% identical to any one of SEQ ID NOs: 12868-12974, at least 80% identical to any one of SEQ ID NOs: 12868-12974, at least 85% identical to of any one of SEQ ID NOs: 12868-12974, at least 90% identical to any one of SEQ ID NOs: 12868-12974, or at least 95% identical to any one of SEQ ID NOs: 12868-12974. In some embodiments, the sense strand sequence comprises or consists of the sequence of any one of SEQ ID NOs: 12868-12974, or a sense strand sequence thereof having 1, 2, 3, or 4 nucleoside substitutions, additions, or deletions. In some embodiments, the sense strand sequence comprises or consists of the sequence of any one of SEQ ID NOs: 12868-12974, or a sense strand sequence thereof having 1 or 2 nucleoside substitutions, additions, or deletions. In some embodiments, the sense strand sequence comprises or consists of a sequence 100% identical to SEQ ID NOs: 12868-12974. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the first 19 nucleotides of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise an overhang. The sense strand may comprise a modification pattern described herein, such as a different set of modifications or modification pattern than the aforementioned sequences. The sense strand may comprise a lipid moiety. The sense strand may comprise a GalNAc moiety. In some embodiments, the siRNA comprises an antisense strand having a sequence in accordance with any of SEQ ID NOs: 12975-13081. In some embodiments, the antisense strand sequence comprises or consists of sequence at least 75% identical to any one of SEQ ID NOs: 12975-13081, at least 80% identical to any one of SEQ ID NOs: 12975-13081, at least 85% identical to of any one of SEQ ID NOs: 12975-13081, at least 90% identical to any one of SEQ ID NOs: 12975-13081, or at least 95% identical to any one of SEQ ID NOs: 12975-13081. In some embodiments, the antisense strand sequence comprises or consists of the sequence of any one of SEQ ID NOs: 12975-13081, or an antisense strand sequence thereof having 1, 2, 3, or 4 nucleoside substitutions, additions, or deletions. In some embodiments, the antisense strand sequence comprises or consists of the sequence of any one of SEQ ID NOs: 12975-13081, or an antisense strand sequence thereof having 1 or 2 nucleoside substitutions, additions, or deletions. In some embodiments, the antisense strand sequence comprises or consists of a sequence 100% identical to SEQ ID NOs: 12975-13081. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the first 19 nucleotides of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein, such as a different set of modifications or modification pattern than the aforementioned sequences. The antisense strand may comprise a lipid moiety or a GalNAc moiety.

In some embodiments, the siRNA comprises a sense strand having a sequence in accordance with any of SEQ ID NOs: 13724-13751. In some embodiments, the sense strand sequence comprises or consists of sequence at least 75% identical to any one of SEQ ID NOs: 13724-13751, at least 80% identical to any one of SEQ ID NOs: 13724-13751, at least 85% identical to of any one of SEQ ID NOs: 13724-13751, at least 90% identical to any one of SEQ ID NOs: 13724-13751, or at least 95% identical to any one of SEQ ID NOs: 13724-13751. In some embodiments, the sense strand sequence comprises or consists of the sequence of any one of SEQ ID NOs: 13724-13751, or a sense strand sequence thereof having 1, 2, 3, or 4 nucleoside substitutions, additions, or deletions. In some embodiments, the sense strand sequence comprises or consists of the sequence of any one of SEQ ID NOs: 13724-13751, or a sense strand sequence thereof having 1 or 2 nucleoside substitutions, additions, or deletions. In some embodiments, the sense strand sequence comprises or consists of a sequence 100% identical to SEQ ID NOs: 13724-13751. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the first 19 nucleotides of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise an overhang. The sense strand may comprise a modification pattern described herein, such as a different set of modifications or modification pattern than the aforementioned sequences. The sense strand may comprise a lipid moiety. The sense strand may comprise a GalNAc moiety. In some embodiments, the siRNA comprises an antisense strand having a sequence in accordance with any of SEQ ID NOs: 13752-13779. In some embodiments, the antisense strand sequence comprises or consists of sequence at least 75% identical to any one of SEQ ID NOs: 13752-13779, at least 80% identical to any one of SEQ ID NOs: 13752-13779, at least 85% identical to of any one of SEQ ID NOs: 13752-13779, at least 90% identical to any one of SEQ ID NOs: 13752-13779, or at least 95% identical to any one of SEQ ID NOs: 13752-13779. In some embodiments, the antisense strand sequence comprises or consists of the sequence of any one of SEQ ID NOs: 13752-13779, or an antisense strand sequence thereof having 1, 2, 3, or 4 nucleoside substitutions, additions, or deletions. In some embodiments, the antisense strand sequence comprises or consists of the sequence of any one of SEQ ID NOs: 13752-13779, or an antisense strand sequence thereof having 1 or 2 nucleoside substitutions, additions, or deletions. In some embodiments, the antisense strand sequence comprises or consists of a sequence 100% identical to SEQ ID NOs: 13752-13779. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the first 19 nucleotides of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein, such as a different set of modifications or modification pattern than the aforementioned sequences. The antisense strand may comprise a lipid moiety or a GalNAc moiety.

In some embodiments, the siRNA comprises a sense strand or antisense strand having a sequence in accordance with the sense strand or antisense strand sequence of an siRNA of subset G. In some embodiments, the sense strand or antisense strand comprises a sequence at least 750% identical, at least 80% identical, at least 85% identical, at least 90% identical, or at least 95% identical, to a sense strand or antisense strand sequence of subset G. In some embodiments, the sense strand or antisense strand comprises a sequence of a sense or antisense strand of subset G, or a sequence thereof having 3 or 4 nucleoside substitutions, additions, or deletions. In some embodiments, the sense strand or antisense strand comprises a sequence of a sense or antisense strand of subset G, or a sequence thereof having 1 or 2 nucleoside substitutions, additions, or deletions. In some embodiments, the sense strand or antisense sequence comprises or consists of a sequence 100% identical to a sense strand or antisense strand sequence of subset G. The sense strand or antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand or antisense strand sequence may include the first 19 nucleotides of any of the aforementioned sequences. The sense strand or antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand or antisense strand may comprise an overhang. The sense strand or antisense strand may comprise any modifications described herein (e.g. a different set of modifications or modification pattern than subset G). The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset H. In some embodiments, the sense strand or antisense strand comprises a sequence at least 750% identical, at least 80% identical, at least 85% identical, at least 90% identical, or at least 95% identical, to a sense strand or antisense strand sequence of subset H. In some embodiments, the sense strand or antisense strand comprises a sequence of a sense or antisense strand of subset H, or a sequence thereof having 3 or 4 nucleoside substitutions, additions, or deletions. In some embodiments, the sense strand or antisense strand comprises a sequence of a sense or antisense strand of subset H, or a sequence thereof having 1 or 2 nucleoside substitutions, additions, or deletions. In some embodiments, the sense strand or antisense sequence comprises or consists of a sequence 100% identical to a sense strand or antisense strand sequence of subset H. The sense strand or antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand or antisense strand sequence may include the first 19 nucleotides of any of the aforementioned sequences. The sense strand or antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand or antisense strand may comprise an overhang. The sense strand or antisense strand may comprise any modifications described herein (e.g. a different set of modifications or modification pattern than subset H). The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 10. In some embodiments, the sense strand or antisense strand comprises a sequence at least 75% identical, at least 80% identical, at least 85% identical, at least 90% identical, or at least 95% identical, to a sense strand or antisense strand sequence of Table 10. In some embodiments, the sense strand or antisense strand comprises a sequence of a sense or antisense strand of Table 10, or a sequence thereof having 3 or 4 nucleoside substitutions, additions, or deletions. In some embodiments, the sense strand or antisense strand comprises a sequence of a sense or antisense strand of Table 10, or a sequence thereof having 1 or 2 nucleoside substitutions, additions, or deletions. In some embodiments, the sense strand or antisense sequence comprises or consists of a sequence

In some embodiments, the siRNA comprises a sense strand or antisense strand having a sequence in accordance with the sense strand or antisense strand sequence of an siRNA of Table 11. In some embodiments, the sense strand or antisense strand comprises a sequence at least 750% identical, at least 80% identical, at least 85% identical, at least 90% identical, or at least 95% identical, to a sense strand or antisense strand sequence of Table 11. In some embodiments, the sense strand or antisense strand comprises a sequence of a sense or antisense strand of Table 11, or a sequence thereof having 3 or 4 nucleoside substitutions, additions, or deletions. In some embodiments, the sense strand or antisense strand comprises a sequence of a sense or antisense strand of Table 11, or a sequence thereof having 1 or 2 nucleoside substitutions, additions, or deletions. In some embodiments, the sense strand or antisense sequence comprises or consists of a sequence 100% identical to a sense strand or antisense strand sequence of Table 11. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 15. In some embodiments, the sense strand or antisense strand comprises a sequence at least 750% identical, at least 80% identical, at least 85% identical, at least 90% identical, or at least 95% identical, to a sense strand or antisense strand sequence of Table 15. In some embodiments, the sense strand or antisense strand comprises a sequence of a sense or antisense strand of Table 15, or a sequence thereof having 3 or 4 nucleoside substitutions, additions, or deletions. In some embodiments, the sense strand or antisense strand comprises a sequence of a sense or antisense strand of Table 15, or a sequence thereof having 1 or 2 nucleoside substitutions, additions, or deletions. In some embodiments, the sense strand or antisense sequence comprises or consists of a sequence. 100% identical to a sense strand or antisense strand sequence of Table 15. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 19. In some embodiments, the sense strand or antisense strand comprises a sequence at least 750% identical, at least 80% identical, at least 85% identical, at least 90% identical, or at least 95% identical, to a sense strand or antisense strand sequence of Table 19. In some embodiments, the sense strand or antisense strand comprises a sequence of a sense or antisense strand of Table 19, or a sequence thereof having 3 or 4 nucleoside substitutions, additions, or deletions. In some embodiments, the sense strand or antisense strand comprises a sequence of a sense or antisense strand of Table 19, or a sequence thereof having 1 or 2 nucleoside substitutions, additions, or deletions. In some embodiments, the sense strand or antisense sequence comprises or consists of a sequence 100% identical to a sense strand or antisense strand sequence of Table 19. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 21. In some embodiments, the sense strand or antisense strand comprises a sequence at least 750% identical, at least 80% identical, at least 85% identical, at least 90% identical, or at least 95% identical, to a sense strand or antisense strand sequence of Table 21. In some embodiments, the sense strand or antisense strand comprises a sequence of a sense or antisense strand of Table 21, or a sequence thereof having 3 or 4 nucleoside substitutions, additions, or deletions. In some embodiments, the sense strand or antisense strand comprises a sequence of a sense or antisense strand of Table 21, or a sequence thereof having 1 or 2 nucleoside substitutions, additions, or deletions. In some embodiments, the sense strand or antisense sequence comprises or consists of a sequence 100% identical to a sense strand or antisense strand sequence of Table 21. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 24. In some embodiments, the sense strand or antisense strand comprises a sequence at least 750% identical, at least 80% identical, at least 85% identical, at least 90% identical, or at least 95% identical, to a sense strand or antisense strand sequence of Table 24. In some embodiments, the sense strand or antisense strand comprises a sequence of a sense or antisense strand of Table 24, or a sequence thereof having 3 or 4 nucleoside substitutions, additions, or deletions. In some embodiments, the sense strand or antisense strand comprises a sequence of a sense or antisense strand of Table 24, or a sequence thereof having 1 or 2 nucleoside substitutions, additions, or deletions. In some embodiments, the sense strand or antisense sequence comprises or consists of a sequence 100% identical to a sense strand or antisense strand sequence of Table 24. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 27. In some embodiments, the sense strand or antisense strand comprises a sequence at least 750% identical, at least 80% identical, at least 85% identical, at least 90% identical, or at least 95% identical, to a sense strand or antisense strand sequence of Table 27. In some embodiments, the sense strand or antisense strand comprises a sequence of a sense or antisense strand of Table 27, or a sequence thereof having 3 or 4 nucleoside substitutions, additions, or deletions. In some embodiments, the sense strand or antisense strand comprises a sequence of a sense or antisense strand of Table 27, or a sequence thereof having 1 or 2 nucleoside substitutions, additions, or deletions. In some embodiments, the sense strand or antisense sequence comprises or consists of a sequence 100% identical to a sense strand or antisense strand sequence of Table 27. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 30. In some embodiments, the sense strand or antisense strand comprises a sequence at least 75% identical, at least 80% identical, at least 85% identical, at least 90% identical, or at least 95% identical, to a sense strand or antisense strand sequence of Table 30. In some embodiments, the sense strand or antisense strand comprises a sequence of a sense or antisense strand of Table 30, or a sequence thereof having 3 or 4 nucleoside substitutions, additions, or deletions. In some embodiments, the sense strand or antisense strand comprises a sequence of a sense or antisense strand of Table 30, or a sequence thereof having 1 or 2 nucleoside substitutions, additions, or deletions. In some embodiments, the sense strand or antisense sequence comprises or consists of a sequence 100% identical to a sense strand or antisense strand sequence of Table 30. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 34. In some embodiments, the sense strand or antisense strand comprises a sequence at least 75% identical, at least 80% identical, at least 85% identical, at least 90% identical, or at least 95% identical, to a sense strand or antisense strand sequence of Table 34. In some embodiments, the sense strand or antisense strand comprises a sequence of a sense or antisense strand of Table 34, or a sequence thereof having 3 or 4 nucleoside substitutions, additions, or deletions. In some embodiments, the sense strand or antisense strand comprises a sequence of a sense or antisense strand of Table 34, or a sequence thereof having 1 or 2 nucleoside substitutions, additions, or deletions. In some embodiments, the sense strand or antisense sequence comprises or consists of a sequence 100% identical to a sense strand or antisense strand sequence of Table 34. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 48. In some embodiments, the sense strand or antisense strand comprises a sequence at least 750% identical, at least 80% identical, at least 85% identical, at least 90% identical, or at least 95% identical, to a sense strand or antisense strand sequence of Table 48. In some embodiments, the sense strand or antisense strand comprises a sequence of a sense or antisense strand of Table 48, or a sequence thereof having 3 or 4 nucleoside substitutions, additions, or deletions. In some embodiments, the sense strand or antisense strand comprises a sequence of a sense or antisense strand of Table 48, or a sequence thereof having 1 or 2 nucleoside substitutions, additions, or deletions. In some embodiments, the sense strand or antisense sequence comprises or consists of a sequence 100% identical to a sense strand or antisense strand sequence of Table 48. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of Table 62. In some embodiments, the sense strand or antisense strand comprises a sequence at least 750% identical, at least 80% identical, at least 85% identical, at least 90% identical, or at least 95% identical, to a sense strand or antisense strand sequence of Table 62. In some embodiments, the sense strand or antisense strand comprises a sequence of a sense or antisense strand of Table 62, or a sequence thereof having 3 or 4 nucleoside substitutions, additions, or deletions. In some embodiments, the sense strand or antisense strand comprises a sequence of a sense or antisense strand of Table 62, or a sequence thereof having 1 or 2 nucleoside substitutions, additions, or deletions. In some embodiments, the sense strand or antisense sequence comprises or consists of a sequence 100% identical to a sense strand or antisense strand sequence of Table 62. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand having a sequence in accordance with any of SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942. In some embodiments, the sense strand sequence comprises or consists of sequence at least 75% identical to any one of SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942, at least 80% identical to any one of SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942, at least 85% identical to of any one of SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942, at least 90% identical to any one of SEQ ID NOs: 13403-13723, or at least 95% identical to any one of SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942. In some embodiments, the sense strand sequence comprises or consists of the sequence of any one of SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942, or a sense strand sequence thereof having 1, 2, 3, or 4 nucleoside substitutions, additions, or deletions. In some embodiments, the sense strand sequence comprises or consists of the sequence of any one of SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942, or a sense strand sequence thereof having 1 or 2 nucleoside substitutions, additions, or deletions. In some embodiments, the sense strand sequence comprises or consists of a sequence 100% identical to SEQ ID NOs: 12953, 12958, 13724, 13725, 13726, 13727, 13728, 13729, 13730, 13731, 13732, 13733, 13734, 13735, 12959, 13736, 13737, 13738, 13739, 13740, 13741, 13742, 13743, 13744, 13745, 13746, 13747, 12877, 12927, 12928, 12929, 12930, 12931, 12932, 12933, 12934, 12960, 13748, 13749, 13750, 13751, 12883, 12910, 12911, 12912, 12913, 12914, 12915, 12916, 12917, 12947, 12948, 12965, 12885, 12901, 12902, 12903, 12904, 12905, 12906, 12907, 12908, 12909, 12886, 12893, 12894, 12895, 12896, 12897, 12898, 12899, 12900, 12968, 12889, 12918, 12919, 12920, 12921, 12922, 12923, 12924, 12925, 12926, 12943, 12944, 12945, 12946, 12969, 12970, 12892, 12935, 12936, 12937, 12938, 12939, 12940, 12941, or 12942. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise an overhang. The sense strand may comprise a modification pattern described herein. The sense strand may comprise a lipid moiety or a GalNAc moiety.

In some embodiments, the siRNA comprises an antisense strand having a sequence in accordance with any of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049. In some embodiments, the antisense strand sequence comprises or consists of sequence at least 75% identical to any one of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049, at least 80% identical to any one of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049, at least 85% identical to of any one of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049, at least 90% identical to any one of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049, or at least 95% identical to any one of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049. In some embodiments, the antisense strand sequence comprises or consists of the sequence ofany one of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049, or an antisense strand sequence thereof having 1, 2, 3, or 4 nucleoside substitutions, additions, or deletions. In some embodiments, the antisense strand sequence comprises or consists of the sequence of any one of SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049, or an antisense strand sequence thereof having 1 or 2 nucleoside substitutions, additions, or deletions. In some embodiments, the antisense strand sequence comprises or consists of a sequence 100% identical to SEQ ID NOs: 13060, 13065, 13752, 13753, 13754, 13755, 13756, 13757, 13758, 13759, 13760, 13761, 13762, 13763, 13066, 13764, 12984, 13034, 13035, 13036, 13037, 13038, 13039, 13040, 13041, 13067, 13765, 13766, 13767, 13768, 12990, 13017, 13018, 13019, 13020, 13021, 13022, 13023, 13024, 13054, 13055, 13072, 12992, 13008, 13009, 13010, 13011, 13012, 13013, 13014, 13015, 13016, 12993, 13000, 13001, 13002, 13003, 13004, 13005, 13006, 13007, 13075, 12996, 13025, 13026, 13027, 13028, 13029, 13030, 13031, 13032, 13033, 13050, 13051, 13052, 13053, 13076, 13077, 12999, 13042, 13043, 13044, 13045, 13046, 13047, 13048, or 13049. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein. The antisense strand may comprise a lipid moiety or a GalNAc moiety.

In some embodiments, the siRNA comprises a sense strand having a sequence in accordance with any of SEQ ID NOs: 12959, 12928, 12932, 12960, 12947, 12965, 12904, 12909, 12897, 12968, 12926, 12969, 12970, 12935, or 12936.. In some embodiments, the sense strand sequence comprises or consists of sequence at least 75% identical to any one of SEQ ID NOs: 12959, 12928, 12932, 12960, 12947, 12965, 12904, 12909, 12897, 12968, 12926, 12969, 12970, 12935, or 12936., at least 80% identical to any one of SEQ ID NOs: 12959, 12928, 12932, 12960, 12947, 12965, 12904, 12909, 12897, 12968, 12926, 12969, 12970, 12935, or 12936., at least 85% identical to of any one of SEQ ID NOs: 12959, 12928, 12932, 12960, 12947, 12965, 12904, 12909, 12897, 12968, 12926, 12969, 12970, 12935, or 12936., at least 90% identical to any one of SEQ ID NOs: 13403-13723, or at least 95% identical to any one of SEQ ID NOs: 12959, 12928, 12932, 12960, 12947, 12965, 12904, 12909, 12897, 12968, 12926, 12969, 12970, 12935, or 12936.. In some embodiments, the sense strand sequence comprises or consists of the sequence of any one of SEQ ID NOs: 12959, 12928, 12932, 12960, 12947, 12965, 12904, 12909, 12897, 12968, 12926, 12969, 12970, 12935, or 12936., or a sense strand sequence thereof having 1, 2, 3, or 4 nucleoside substitutions, additions, or deletions. In some embodiments, the sense strand sequence comprises or consists of the sequence of any one of SEQ ID NOs: 12959, 12928, 12932, 12960, 12947, 12965, 12904, 12909, 12897, 12968, 12926, 12969, 12970, 12935, or 12936., or a sense strand sequence thereof having 1 or 2 nucleoside substitutions, additions, or deletions. In some embodiments, the sense strand sequence comprises or consists of a sequence 100% identical to SEQ ID NOs: 12959, 12928, 12932, 12960, 12947, 12965, 12904, 12909, 12897, 12968, 12926, 12969, 12970, 12935, or 12936.. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise an overhang. The sense strand may comprise a modification pattern described herein. The sense strand may comprise a lipid moiety or a GalNAc moiety.

In some embodiments, the siRNA comprises an antisense strand having a sequence in accordance with any of SEQ ID NOs: 13060, 13065, 13066, 13035, 13039, 13067, 13054, 13072, 13011, 13016, 13004, 13075, 13033, 13076, 13077, 13042, or 13043. In some embodiments, the antisense strand sequence comprises or consists of sequence at least 75% identical to any one of SEQ ID NOs: 13060, 13065, 13066, 13035, 13039, 13067, 13054, 13072, 13011, 13016, 13004, 13075, 13033, 13076, 13077, 13042, or 13043, at least 80% identical to any one of SEQ ID NOs: 13060, 13065, 13066, 13035, 13039, 13067, 13054, 13072, 13011, 13016, 13004, 13075, 13033, 13076, 13077, 13042, or 13043, at least 85% identical to of any one of SEQ ID NOs: 13060, 13065, 13066, 13035, 13039, 13067, 13054, 13072, 13011, 13016, 13004, 13075, 13033, 13076, 13077, 13042, or 13043, at least 90% identical to any one of SEQ ID NOs: 13060, 13065, 13066, 13035, 13039, 13067, 13054, 13072, 13011, 13016, 13004, 13075, 13033, 13076, 13077, 13042, or 13043, or at least 95% identical to any one of SEQ ID NOs: 13060, 13065, 13066, 13035, 13039, 13067, 13054, 13072, 13011, 13016, 13004, 13075, 13033, 13076, 13077, 13042, or 13043. In some embodiments, the antisense strand sequence comprises or consists of the sequence of any one of SEQ ID NOs: 13060, 13065, 13066, 13035, 13039, 13067, 13054, 13072, 13011, 13016, 13004, 13075, 13033, 13076, 13077, 13042, or 13043, or an antisense strand sequence thereof having 1, 2, 3, or 4 nucleoside substitutions, additions, or deletions. In some embodiments, the antisense strand sequence comprises or consists of the sequence of any one of SEQ ID NOs: 13060, 13065, 13066, 13035, 13039, 13067, 13054, 13072, 13011, 13016, 13004, 13075, 13033, 13076, 13077, 13042, or 13043, or an antisense strand sequence thereof having 1 or 2 nucleoside substitutions, additions, or deletions. In some embodiments, the antisense strand sequence comprises or consists of a sequence 100% identical to SEQ ID NOs: 13060, 13065, 13066, 13035, 13039, 13067, 13054, 13072, 13011, 13016, 13004, 13075, 13033, 13076, 13077, 13042, or 13043. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein. The antisense strand may comprise a lipid moiety or a GalNAc moiety. 4. Modified ASOs

In some embodiments, the composition comprises an oligonucleotide that inhibits the expression of GPAM, wherein the oligonucleotide comprises an antisense oligonucleotide (ASO). In some embodiments, the ASO comprises modification pattern ASO1: 5′-nsnsnsnsnsdNsdNsdNsdNsdNsdNsdNsdNsdNsdNsnsnsnsnsn-3′ (SEQ ID NO: 12928), wherein “dN” is any deoxynucleotide, “n” is a 2′O-methyl or 2′-O-(2-methoxyethyl)-modified nucleoside, and “s” is a phosphorothioate linkage. In some embodiments, the ASO comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 52S, 54S, 55S, 56S, 57S, 58S, 59S, 60S, 61S, 62S, 63S, 64S, 65S, 66S, 67S, 68S, 69S, 70S, 1AS, 2AS, 3AS, 4AS, 5AS, 6AS, 7AS, or 8AS, 9AS, 10AS, 11AS, 12AS, 13AS, 14AS, 15AS, 16AS, 17AS, 18AS, 19AS, 20AS, 21AS, 22AS, 23AS, 24AS, 25AS, 26AS, 27AS, 28AS, 29AS, 30AS, 31AS, or 32AS.

### D. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof

### II. METHODS AND USES

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject. The disorder may comprise a disease.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection.

### A. Disorders

Some embodiments of the methods described herein include treating a disorder in a subject in need thereof. In some embodiments, the disorder is a liver disease. Non-limiting examples of liver diseases may include non-alcoholic fatty liver disease (NAFLD), non-alcoholic steatohepatitis (NASH), alcoholic liver disease, liver fibrosis, liver cirrhosis, or hepatocellular carcinoma. In some embodiments, the disorder is a cardiometabolic disease. Non-limiting examples of cardiometabolic diseases may include hyperlipidemia, ischemic heart disease, or coronary heart disease. In some embodiments, the disorder comprises NAFLD. In some embodiments, the disorder comprises NASH. In some embodiments, the disorder comprises alcoholic liver disease. In some embodiments, the disorder comprises liver fibrosis. In some embodiments, the disorder comprises liver cirrhosis. In some embodiments, the disorder comprises hepatocellular carcinoma. In some embodiments, the disorder comprises hyperlipidemia. In some embodiments, the disorder comprises a heart disease. In some embodiments, the heart disease comprises ischemic heart disease. In some embodiments, the heart disease comprises coronary heart disease.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female. In some embodiments, the subject is an adult (e.g. at least 18 years old).

In some embodiments, the subject has a body mass index (BMI) of 25, 26, 27, 28, 29, 30, 31, 32, 33, 34, 35, 36, 37, 38, 39, 40, 41, 42, 43, 44, 45, 46, 47, 48, 49, 50, or more, or a range defined by any two of the aforementioned integers. In some embodiments, the subject is overweight. In some embodiments, the subject has a BMI of 25 or more. In some embodiments, the subject has a BMI of 25-29. In some embodiments, the subject is obese. In some embodiments, the subject has a BMI of 30 or more. In some embodiments, the subject has a BMI of 30-39. In some embodiments, the subject has a BMI of 40-50. In some embodiments, the subject has a BMI of 25-50.

### C. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline liver fat percentage measurement, a baseline liver fibrosis score measurement, a baseline NAFLD activity score measurement, a baseline blood alanine aminotransferase (ALT) measurement, a baseline blood aspartate aminotransferase (AST) measurement, a baseline blood aspartate aminotransferase (ASP) measurement, a baseline blood bilirubin measurement, a baseline low-density lipoprotein (LDL) measurement, a baseline total cholesterol measurement, a baseline non-HDL cholesterol measurement, a baseline apolipoprotein B (APOB), a baseline GPAM protein measurement, or a baseline GPAM mRNA measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device.

In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline liver fat percentage measurement. In some embodiments, the baseline liver fat percentage measurement is a baseline percentage as measured by MRI. In some embodiments, the baseline liver fat percentage measurement is a baseline percentage as measured by CT scan. In some embodiments, the baseline liver fat percentage is measured in a liver biopsy.

In some embodiments, the baseline measurement is a baseline NAFLD activity score measurement. In some embodiments, the baseline NAFLD activity score measurement is measured by histological analysis. In some embodiments, the baseline NAFLD activity score measurement includes a numerical score for steatosis (0-3), hepatocyte ballooning (1-2), and lobular inflammation (0-3). In some embodiments, the score thresholds of <3 correlates with a diagnosis of not-NASH. In some embodiments, the score thresholds of >5 correlates with a diagnosis of NASH. In some embodiments, a baseline NAFLD activity score measurement is assayed by imaging.

In some embodiments, the baseline measurement is a baseline alanine aminotransferase (ALT) measurement. In some embodiments, the baseline ALT measurement includes a baseline blood ALT measurement. In some embodiments, the baseline ALT measurement is measured by enzymatic activity. In some embodiments, the baseline ALT measurement is measured by presence of an ALT epitope. In some embodiments, the baseline ALT measurement is a baseline circulating ALT measurement. In some embodiments, the baseline ALT measurement is obtained by an assay such as an immunoassay, a colorimetric assay, or a fluorescence assay.

In some embodiments, the baseline measurement is a baseline aspartate aminotransferase (AST) measurement. In some embodiments, the baseline AST measurement includes a baseline blood AST measurement. In some embodiments, the baseline AST measurement is measured by enzymatic activity. In some embodiments, the baseline AST measurement is measured by presence of an AST epitope. In some embodiments, the baseline AST measurement is a baseline circulating AST measurement. In some embodiments, the baseline AST measurement is obtained by an assay such as an immunoassay, a colorimetric assay, or a fluorescence assay.

In some embodiments, the baseline measurement is a baseline aspartate aminotransferase (ASP) measurement. In some embodiments, the baseline ASP measurement includes a baseline blood ASP measurement. In some embodiments, the baseline ASP measurement is measured by enzymatic activity. In some embodiments, the baseline ASP measurement is measured by presence of an ASP epitope. In some embodiments, the baseline ASP measurement is a baseline circulating ASP measurement. In some embodiments, the baseline ASP measurement is obtained by an assay such as an immunoassay, a colorimetric assay, or a fluorescence assay.

In some embodiments, the baseline measurement is a baseline bilirubin measurement. In some embodiments, the baseline bilirubin measurement includes a baseline blood bilirubin measurement. In some embodiments, the baseline bilirubin measurement is measured by presence of a bilirubin epitope. In some embodiments, the baseline bilirubin measurement is a baseline circulating bilirubin measurement. In some embodiments, the baseline bilirubin measurement is obtained by an assay such as an immunoassay, a colorimetric assay, or a fluorescence assay.

In some embodiments, the baseline measurement is a baseline low-density lipoprotein (LDL) measurement. In some embodiments, the baseline LDL measurement includes a baseline blood LDL measurement. In some embodiments, the baseline LDL measurement is measured by presence of an LDL epitope. In some embodiments, the baseline LDL measurement is a baseline circulating LDL measurement. In some embodiments, the baseline LDL measurement is obtained by an assay such as an immunoassay, a colorimetric assay, or a fluorescence assay.

In some embodiments, the baseline measurement is a baseline total cholesterol measurement. In some embodiments, the baseline total cholesterol measurement includes a baseline blood total cholesterol measurement. In some embodiments, the baseline total cholesterol is measured by presence of a total cholesterol epitope. In some embodiments, the baseline total cholesterol measurement is a baseline circulating total cholesterol measurement. In some embodiments, the baseline total cholesterol measurement is obtained by an assay such as an immunoassay, a colorimetric assay, or a fluorescence assay.

In some embodiments, the baseline measurement is a baseline non-HDL cholesterol measurement. In some embodiments, the baseline non-HDL cholesterol measurement includes a baseline blood non-HDL cholesterol measurement. In some embodiments, the baseline non-HDL cholesterol measurement is a baseline circulating non-HDL cholesterol measurement. In some embodiments, the baseline non-HDL cholesterol measurement is obtained by an assay such as an immunoassay, a colorimetric assay, or a fluorescence assay.

In some embodiments, the baseline measurement is a baseline apolipoprotein B (APOB) measurement. In some embodiments, the baseline APOB includes a baseline blood APOB measurement. In some embodiments, the baseline APOB is measured by presence of an APOB epitope. In some embodiments, the baseline APOB measurement is a baseline circulating APOB measurement. In some embodiments, the baseline APOB measurement is obtained by an assay such as an immunoassay, a colorimetric assay, or a fluorescence assay.

In some embodiments, the baseline measurement is a baseline liver fibrosis measurement. In some embodiments, the baseline liver fibrosis measurement is a baseline liver fibrosis score (LFS). In some embodiments, the baseline LFS comprises a score of 0, 1, 2, 3, or 4, or a range of scores defined by any two of the aforementioned numbers. In some embodiments, the baseline LFS comprises a score of 0-4. In some embodiments, the baseline LFS is obtained using a scoring system exemplified in Table 2. In some embodiments, the baseline LFS measurement is obtained noninvasively. In some embodiments, the baseline LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the baseline LFS measurement is obtained in a liver sample. In some embodiments, the baseline LFS is obtained by an assay such as an immunoassay, a colorimetric assay, or a fluorescence assay. In some embodiments, the baseline LFS is obtained using one or more indirect markers or measures of liver fibrosis such as an aspartate aminotransferase-to-platelet ratio index (APRI), a Fibrosis-4 (FIB-4) index, a FibroIndex, a Forns Index, a Hepascore, or a FibroTest. In some embodiments, the baseline LFS is obtained using one or more indirect markers or measures of liver fibrosis such as a FIBROSpect test or a FIBROSpect II test. In some embodiments, the baseline LFS is obtained by RT-qPCR or RNA sequencing of one or more fibrosis-related genes such as a collagen gene. In some embodiments, the baseline LFS or the baseline LFS is obtained using a scoring system upon a visual inspection of a sample such as a histological sample. In some embodiments, the baseline LFS or the baseline LFS is obtained using a stain with an affinity to collagen. In some embodiments, the baseline LFS measurement is measured by FibroScan® in kilopascals (kPa). In some embodiments, the baseline LFS is measured by histological analysis. In some embodiments, the baseline LFS measurement is a baseline LFS measurement assayed by imaging. In some embodiments, the imaging is by MRI or CT scan.

TABLE 2
Non-Limiting Examples of Liver Fibrosis Scoring Systems

| Score | IASL | Batts-Ludwig | Metavir |
|---|---|---|---|
| 0 | No fibrosis | No fibrosis | No fibrosis |
| 1 | Mild fibrosis | Fibrous portal expansion | Periportal fibrotic expansion |
| 2 | Moderate fibrosis | Rare bridges or septae | Periportal septae (>1 septum) |
| 3 | Severe fibrosis | Numerous bridges or septae | Portal-central septae |
| 4 | Cirrhosis | Cirrhosis | Cirrhosis |

In some embodiments, the baseline measurement is a baseline GPAM mRNA measurement. In some embodiments, the baseline GPAM mRNA measurement comprises a baseline GPAM mRNA level. In some embodiments, the baseline GPAM mRNA level is indicated as an amount or percentage of GPAM mRNA per sample weight. In some embodiments, the baseline GPAM mRNA level is indicated as an amount or percentage of GPAM mRNA per sample volume. In some embodiments, the baseline GPAM mRNA level is indicated as an amount or percentage of GPAM mRNA per total mRNA within the sample. In some embodiments, the baseline GPAM mRNA level is indicated as an amount or percentage of GPAM mRNA per total nucleic acids within the sample. In some embodiments, the baseline GPAM mRNA level is indicated relative to another mRNA level, such as an mRNA level of a housekeeping gene, within the sample. In some embodiments, the baseline GPAM mRNA measurement is a baseline tissue GPAM mRNA measurement. In some embodiments, the baseline GPAM mRNA measurement is obtained by an assay such as a polymerase chain reaction (PCR) assay. In some embodiments, the PCR comprises quantitative PCR (qPCR). In some embodiments, the PCR comprises reverse transcription of the GPAM mRNA.

In some embodiments, the baseline measurement is a baseline GPAM protein measurement. In some embodiments, the baseline GPAM protein measurement comprises a baseline GPAM protein level. In some embodiments, the baseline GPAM protein level is indicated as a mass or percentage of GPAM protein per sample weight. In some embodiments, the baseline GPAM protein level is indicated as a mass or percentage of GPAM protein per sample volume. In some embodiments, the baseline GPAM protein level is indicated as a mass or percentage of GPAM protein per total protein within the sample. In some embodiments, the baseline GPAM protein measurement is a baseline tissue GPAM protein measurement. In some embodiments, the baseline GPAM protein measurement is obtained by an assay such as an immunoassay, a colorimetric assay, or a fluorescence assay.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample is obtained from the subject in a fasted state. In some embodiments, the sample is obtained from the subject after an overnight fasting period. In some embodiments, the sample is obtained from the subject in a fed state.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. A blood sample may be a plasma sample. In some embodiments, the sample comprises serum. In some embodiments, the sample is a serum sample. A blood sample may be a serum sample.

In some embodiments, the sample comprises a tissue. In some embodiments, the sample is a tissue sample. In some embodiments, the tissue comprises liver or adipose tissue. For example, the baseline GPAM mRNA measurement, or the baseline GPAM protein measurement, may be obtained in a liver or adipose sample obtained from the patient. In some embodiments, the tissue comprises adipose tissue. In some embodiments, the adipose tissue comprises white adipose tissue. The adipose tissue may include adipocytes. The adipose tissue may include preadipocytes In some embodiments, the tissue comprises liver tissue. The liver may include hepatocytes. In some embodiments, the tissue comprises vasculature tissue. In some embodiments, the vasculature tissue comprises endothelial cells.

In some embodiments, the sample includes cells. In some embodiments, the sample comprises a cell. In some embodiments, the cell comprises an adipose cell or a liver cell. In some embodiments, the cell is an adipose cell. In some embodiments, the adipose cell is an adipocyte. In some embodiments, the cell is a liver cell. In some embodiments, the liver cell is a hepatocyte. In some embodiments, the cell is a vasculature cell. In some embodiments, the vasculature cell is an endothelial cell.

### D. Effects

In some embodiments, the composition or administration of the composition affects a measurement such as a liver fat percentage measurement, a liver fibrosis score measurement, a NAFLD activity score measurement, a blood alanine aminotransferase (ALT) measurement, a blood aspartate aminotransferase (AST) measurement, a blood aspartate aminotransferase (ASP) measurement, a blood bilirubin measurement, a low-density lipoprotein (LDL) measurement, a total cholesterol measurement, a non-HDL cholesterol measurement, an apolipoprotein B (APOB), a GPAM protein measurement, or a GPAM mRNA measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, a chromatography (e.g. HPLC) assay, or a PCR assay. In some embodiments, the measurement is obtained by an assay such as an immunoassay, a colorimetric assay, a fluorescence assay, or a chromatography (e.g. HPLC) assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. For example, an adverse phenotype of NAFLD, NASH, alcoholic liver disease, liver fibrosis, liver cirrhosis, hepatocellular carcinoma, hyperlipidemia, ischemic heart disease, or coronary heart disease may be reduced upon administration of the composition. In some embodiments, the reduction is measured in a second sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. For example, a protective liver function phenotype, metabolic phenotype, or level of circulating ketone bodies may be increased upon administration of the composition. In some embodiments, the increase is measured in a second sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a liver fat percentage (LFP) measurement. In some embodiments, the LFP measurement is a percentage as measured by MRI. In some embodiments, the LFP measurement is a percentage as measured by CT scan. In some embodiments, the LFP is measured in a liver biopsy.

In some embodiments, the composition reduces the LFP measurement relative to the baseline LFP measurement. In some embodiments, the LFP measurement is decreased by about 2.5% or more, about 5% or more, or about 7.5% or more, relative to the baseline LFP measurement. In some embodiments, the LFP measurement is decreased by about 10% or more, relative to the baseline LFP measurement. In some embodiments, the LFP measurement is decreased by about 20% or more, about 30% or more, about 40% or more, about 50% or more, about 60% or more, about 70% or more, about 80% or more, about 90% or more, or about 100% or more relative to the baseline LFP measurement. In some embodiments, the LFP measurement is decreased by no more than about 2.5%, no more than about 5%, or no more than about 7.5%, relative to the baseline LFP measurement. In some embodiments, the LFP measurement is decreased by no more than about 10%, relative to the baseline LFP measurement. In some embodiments, the LFP measurement is decreased by no more than about 20%, no more than about 30%, no more than about 40%, no more than about 50%, no more than about 60%, no more than about 70%, no more than about 80%, no more than about 90%, or about 100% relative to the baseline LFP measurement. In some embodiments, the LFP measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a liver fibrosis measurement. In some embodiments, the liver fibrosis measurement is a liver fibrosis score (LFS). In some embodiments, the LFS comprises a score of 0, 1, 2, 3, or 4, or a range of scores defined by any two of the aforementioned numbers. In some embodiments, the LFS comprises a score of 0-4. In some embodiments, the LFS is obtained using a scoring system exemplified in Table 2. In some embodiments, the LFS measurement is obtained noninvasively. In some embodiments, the LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the LFS measurement is obtained in a liver sample. In some embodiments, the LFS is obtained by an assay such as an immunoassay, a colorimetric assay, or a fluorescence assay. In some embodiments, the LFS is obtained using one or more indirect markers or measures of liver fibrosis such as an aspartate aminotransferase-to-platelet ratio index (APRI), a Fibrosis-4 (FIB-4) index, a FibroIndex, a Forns Index, a Hepascore, or a FibroTest. In some embodiments, the LFS is obtained using one or more indirect markers or measures of liver fibrosis such as a FIBROSpect test or a FIBROSpect II test. In some embodiments, the LFS is obtained by RT-qPCR or RNA sequencing of one or more fibrosis-related genes such as a collagen gene. In some embodiments, the LFS or the LFS is obtained using a scoring system upon a visual inspection of a sample such as a histological sample. In some embodiments, the LES or the LFS is obtained using a stain with an affinity to collagen. In some embodiments, the LFS measurement is measured by FibroScan® in kilopascals (kPa). In some embodiments, the LFS is measured by histological analysis. In some embodiments, the LES measurement is a LES measurement assayed by imaging. In some embodiments, the imaging is by MRI or CT scan.

In some embodiments, the composition reduces the LFS measurement relative to the baseline LFS measurement. In some embodiments, the LUS measurement is decreased by about 2.5% or more, about 5% or more, or about 7.5% or more, relative to the baseline LFS measurement. In some embodiments, the LFS measurement is decreased by about 10% or more, relative to the baseline LFS measurement. In some embodiments, the LFS measurement is decreased by about 20% or more, about 30% or more, about 40% or more, about 50% or more, about 60% or more, about 70% or more, about 80% or more, about 90% or more, or about 100% or more relative to the baseline LFS measurement. In some embodiments, the LFS measurement is decreased by no more than about 2.5%, no more than about 5%, or no more than about 7.5%, relative to the baseline LFS measurement. In some embodiments, the LFS measurement is decreased by no more than about 10%, relative to the baseline LFS measurement. In some embodiments, the LFS measurement is decreased by no more than about 20%, no more than about 30%, no more than about 40%, no more than about 50%, no more than about 60%, no more than about 70%, no more than about 80%, no more than about 90%, or about 100% relative to the baseline LFS measurement. In some embodiments, the LES measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a NAFLD activity score measurement. In some embodiments, the NAFLD activity score measurement is measured by histological analysis. In some embodiments, the NAFLD activity score measurement includes a numerical score for steatosis (0-3), hepatocyte ballooning (1-2), and lobular inflammation (0-3). In some embodiments, the score thresholds of <3 correlates with a diagnosis of not-NASH. In some embodiments, the score thresholds of >5 correlates with a diagnosis of NASH. In some embodiments, a NAFLD activity score measurement is assayed by imaging.

In some embodiments, the composition reduces the NAFLD activity score measurement relative to the baseline NAFLD activity score measurement. In some embodiments, the NAFLD activity score measurement is decreased by about 2.5% or more, about 5% or more, or about 7.5% or more, relative to the baseline NAFLD activity score measurement. In some embodiments, the NAFLD activity score measurement is decreased by about 10% or more, relative to the baseline NAFLD activity score measurement. In some embodiments, the NAFLD activity score measurement is decreased by about 20% or more, about 30% or more, about 40% or more, about 50% or more, about 60% or more, about 70% or more, about 80% or more, about 90% or more, or about 100% or more relative to the baseline NAFLD activity score measurement. In some embodiments, the NAFLD ACTIVITY SCORE measurement is decreased by no more than about 2.5%, no more than about 5%, or no more than about 7.5%, relative to the baseline NAFLD ACTIVITY SCORE measurement. In some embodiments, the NAFLD ACTIVITY SCORE measurement is decreased by no more than about 10%, relative to the baseline NAFLD ACTIVITY SCORE measurement. In some embodiments, the NAFLD ACTIVITY SCORE measurement is decreased by no more than about 20%, no more than about 30%, no more than about 40%, no more than about 50%, no more than about 60%, no more than about 70%, no more than about 80%, no more than about 90%, or about 100% relative to the baseline NAFLD ACTIVITY SCORE measurement. In some embodiments, the NAFLD ACTIVITY SCORE measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an alanine aminotransferase (ALT) measurement. In some embodiments, the ALT measurement includes a blood ALT measurement. In some embodiments, the ALT measurement is measured by enzymatic activity. In some embodiments, the ALT measurement is measured by presence of an ALT epitope. In some embodiments, the ALT measurement is a circulating ALT measurement. In some embodiments, the ALT measurement is obtained by an assay such as an immunoassay, a colorimetric assay, or a fluorescence assay. In some embodiments, the ALT measurement is a blood ALT concentration (for example, units per liter (U/L)). In some embodiments, the ALT measurement is a circulating ALT measurement. In some embodiments, the ALT measurement is obtained by an assay such as an immunoassay, a colorimetric assay, or a fluorescence assay.

In some embodiments, the composition reduces the ALT measurement relative to the baseline ALT measurement. In some embodiments, the composition reduces circulating ALTs relative to the baseline ALT measurement. In some embodiments, the reduced ALTs are measured in a second sample obtained from the subject after administering the composition to the subject. In some embodiments, the ALT measurement is decreased by about 2.5% or more, about 5% or more, or about 7.5% or more, relative to the baseline ALT measurement. In some embodiments, the ALT measurement is decreased by about 10% or more, relative to the baseline ALT measurement. In some embodiments, the ALT measurement is decreased by about 20% or more, about 30% or more, about 40% or more, about 50% or more, about 60% or more, about 70% or more, about 80% or more, about 90% or more, or about 100% or more relative to the baseline ALT measurement. In some embodiments, the ALT measurement is decreased by no more than about 2.5%, no more than about 5%, or no more than about 7.5%, relative to the baseline ALT measurement. In some embodiments, the ALT measurement is decreased by no more than about 10%, relative to the baseline ALT measurement. In some embodiments, the ALT measurement is decreased by no more than about 20%, no more than about 30%, no more than about 40%, no more than about 50%, no more than about 60%, no more than about 70%, no more than about 80%, no more than about 90%, or about 100% relative to the baseline ALT measurement. In some embodiments, the ALT measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an aspartate aminotransferase (AST) measurement. In some embodiments, the AST measurement includes a blood AST measurement. In some embodiments, the AST measurement is measured by enzymatic activity. In some embodiments, the AST measurement is measured by presence of an AST epitope. In some embodiments, the AST measurement is a blood AST concentration (for example, units per liter (U/L)). In some embodiments, the AST measurement is a circulating AST measurement. In some embodiments, the AST measurement is obtained by an assay such as an immunoassay, a colorimetric assay, or a fluorescence assay.

In some embodiments, the composition reduces the AST measurement relative to the baseline AST measurement. In some embodiments, the composition reduces circulating ASTs relative to the baseline AST measurement. In some embodiments, the reduced ASTs are measured in a second sample obtained from the subject after administering the composition to the subject. In some embodiments, the AST measurement is decreased by about 2.5% or more, about 5% or more, or about 7.5% or more, relative to the baseline AST measurement. In some embodiments, the AST measurement is decreased by about 10% or more, relative to the baseline AST measurement. In some embodiments, the AST measurement is decreased by about 20% or more, about 30% or more, about 40% or more, about 50% or more, about 60% or more, about 70% or more, about 80% or more, about 90% or more, or about 100% or more relative to the baseline AST measurement. In some embodiments, the AST measurement is decreased by no more than about 2.5%, no more than about 5%, or no more than about 7.5%, relative to the baseline AST measurement. In some embodiments, the AST measurement is decreased by no more than about 10%, relative to the baseline AST measurement. In some embodiments, the AST measurement is decreased by no more than about 20%, no more than about 30%, no more than about 40%, no more than about 50%, no more than about 60%, no more than about 70%, no more than about 80%, no more than about 90%, or about 100% relative to the baseline AST measurement. In some embodiments, the AST measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an aspartate aminotransferase (ASP) measurement. In some embodiments, the ASP measurement includes a blood ASP measurement. In some embodiments, the ASP measurement is a blood ASP concentration (for example, units per liter (U/L)). In some embodiments, the ASP measurement is measured by enzymatic activity. In some embodiments, the ASP measurement is measured by presence of an ASP epitope. In some embodiments, the ASP measurement is a circulating ASP measurement. In some embodiments, the ASP measurement is obtained by an assay such as an immunoassay, a colorimetric assay, or a fluorescence assay.

In some embodiments, the composition reduces the ASP measurement relative to the baseline ASP measurement. In some embodiments, the composition reduces circulating ASPs relative to the baseline ASP measurement. In some embodiments, the reduced ASPs are measured in a second sample obtained from the subject after administering the composition to the subject. In some embodiments, the ASP measurement is decreased by about 2.5% or more, about 5% or more, or about 7.5% or more, relative to the baseline ASP measurement. In some embodiments, the ASP measurement is decreased by about 10% or more, relative to the baseline ASP measurement. In some embodiments, the ASP measurement is decreased by about 20% or more, about 30% or more, about 40% or more, about 50% or more, about 60% or more, about 70% or more, about 80% or more, about 90% or more, or about 100% or more relative to the baseline ASP measurement. In some embodiments, the ASP measurement is decreased by no more than about 2.5%, no more than about 5%, or no more than about 7.5%, relative to the baseline ASP measurement. In some embodiments, the ASP measurement is decreased by no more than about 10%, relative to the baseline ASP measurement. In some embodiments, the ASP measurement is decreased by no more than about 20%, no more than about 30%, no more than about 40%, no more than about 50%, no more than about 60%, no more than about 70%, no more than about 80%, no more than about 90%, or about 100% relative to the baseline ASP measurement. In some embodiments, the ASP measurement is decreased by 2.5%, 5%, 7.5%0, 10%, 20%, 30%, 40%, 50%, 60%, 70%, 80%, 90%, or 100%, or by a range defined by any of the two aforementioned percentages.

In some embodiments, the measurement is a bilirubin measurement. In some embodiments, the bilirubin measurement includes a blood bilirubin measurement. In some embodiments, the bilirubin measurement is a blood bilirubin concentration (for example, units per liter (pmol/L)). In some embodiments, the bilirubin measurement is measured by presence of a bilirubin epitope. In some embodiments, the bilirubin measurement is a circulating bilirubin measurement. In some embodiments, the bilirubin measurement is obtained by an assay such as an immunoassay, a colorimetric assay, or a fluorescence assay.

In some embodiments, the composition reduces the bilirubin measurement relative to the baseline bilirubin measurement. In some embodiments, the composition reduces circulating bilirubin relative to the baseline bilirubin measurement. In some embodiments, the reduced bilirubin is measured in a second sample obtained from the subject after administering the composition to the subject. In some embodiments, the bilirubin measurement is decreased by about 2.5% or more, about 5% or more, or about 7.5% or more, relative to the baseline bilirubin measurement. In some embodiments, the bilirubin measurement is decreased by about 10% or more, relative to the baseline bilirubin measurement. In some embodiments, the bilirubin measurement is decreased by about 20% or more, about 30% or more, about 40% or more, about 50% or more, about 60% or more, about 70% or more, about 80% or more, about 90% or more, or about 100% or more relative to the baseline bilirubin measurement. In some embodiments, the bilirubin measurement is decreased by no more than about 2.5%, no more than about 5%, or no more than about 7.5%, relative to the baseline bilirubin measurement. In some embodiments, the bilirubin measurement is decreased by no more than about 10%, relative to the baseline bilirubin measurement. In some embodiments, the bilirubin measurement is decreased by no more than about 20%, no more than about 30%, no more than about 40%, no more than about 50%, no more than about 60%, no more than about 70%, no more than about 80%, no more than about 90%, or about 100% relative to the baseline bilirubin measurement. In some embodiments, the bilimbin measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a low-density lipoprotein (LDL) measurement. In some embodiments, the LDL measurement includes a blood LDL measurement. In some embodiments, the LDL measurement is a blood LDL concentration (for example, units per liter (mg/dL)). In some embodiments, the LDL measurement is measured by presence of an LDL epitope. In some embodiments, the LDL measurement is a circulating LDL measurement. In some embodiments, the LDL measurement is obtained by an assay such as an immunoassay, a colorimetric assay, or a fluorescence assay.

In some embodiments, the composition reduces the LDL measurement relative to the baseline LDL measurement. In some embodiments, the composition reduces circulating LDLs relative to the baseline LDL measurement. In some embodiments, the reduced LDLs are measured in a second sample obtained from the subject after administering the composition to the subject. In some embodiments, the LDL measurement is decreased by about 2.5% or more, about 5% or more, or about 7.5% or more, relative to the baseline LDL measurement. In some embodiments, the LDL measurement is decreased by about 10% or more, relative to the baseline LDL measurement. In some embodiments, the LDL measurement is decreased by about 20% or more, about 30% or more, about 40% or more, about 50% or more, about 60% or more, about 70% or more, about 80% or more, about 90% or more, or about 100% or more relative to the baseline LDL measurement. In some embodiments, the LDL measurement is decreased by no more than about 2.5%, no more than about 5%, or no more than about 7.5%, relative to the baseline LDL measurement. In some embodiments, the LDL measurement is decreased by no more than about 10%, relative to the baseline LDL measurement. In some embodiments, the LDL measurement is decreased by no more than about 20%, no more than about 30%, no more than about 40%, no more than about 50%, no more than about 60%, no more than about 70%, no more than about 80%, no more than about 90%, or about 100% relative to the baseline LDL measurement. In some embodiments, the LDL measurement is decreased by 2.5%, 5%, 7.5%0, 10%, 20%, 30%, 40%, 50%, 60%, 70%, 80%, 90%, or 100%, or by a range defined by any of the two aforementioned percentages.

In some embodiments, the measurement is an APOB measurement. In some embodiments, the APOB measurement is a blood APOB concentration (for example, units per liter (mg/dL)). In some embodiments, the APOB measurement is a circulating APOB measurement. In some embodiments, the APOB measurement is obtained by an assay such as an immunoassay, a colorimetric assay, or a fluorescence assay.

In some embodiments, the composition reduces the APOB measurement relative to the baseline APOB measurement. In some embodiments, the composition reduces circulating APOBs relative to the baseline APOB measurement. In some embodiments, the reduced APOBs are measured in a second sample obtained from the subject after administering the composition to the subject. In some embodiments, the APOB measurement is decreased by about 2.5% or more, about 5% or more, or about 7.5% or more, relative to the baseline APOB measurement. In some embodiments, the APOB measurement is decreased by about 10% or more, relative to the baseline APOB measurement. In some embodiments, the APOB measurement is decreased by about 20% or more, about 30% or more, about 40% or more, about 50% or more, about 60% or more, about 70% or more, about 80% or more, about 90% or more, or about 100% or more relative to the baseline APOB measurement. In some embodiments, the APOB measurement is decreased by no more than about 2.5%, no more than about 5%, or no more than about 7.5%, relative to the baseline APOB measurement. In some embodiments, the APOB measurement is decreased by no more than about 10%, relative to the baseline APOB measurement. In some embodiments, the APOB measurement is decreased by no more than about 20%, no more than about 30%, no more than about 40%, no more than about 50%, no more than about 60%, no more than about 70%, no more than about 80%, no more than about 90%, or about 100% relative to the baseline APOB measurement. In some embodiments, the APOB measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a total cholesterol measurement. In some embodiments, the total cholesterol measurement is a blood total cholesterol concentration (for example, units per liter (mg/dL)). In some embodiments, the total cholesterol measurement is a circulating total cholesterol measurement. In some embodiments, the total cholesterol measurement is obtained by an assay such as an immunoassay, a colorimetric assay, or a fluorescence assay.

In some embodiments, the composition reduces the total cholesterol measurement relative to the baseline total cholesterol measurement. In some embodiments, the composition reduces circulating total cholesterol relative to the baseline total cholesterol measurement. In some embodiments, the reduced total cholesterol is measured in a second sample obtained from the subject after administering the composition to the subject. In some embodiments, the total cholesterol measurement is decreased by about 2.5% or more, about 5% or more, or about 7.5% or more, relative to the baseline total cholesterol measurement. In some embodiments, the total cholesterol measurement is decreased by about 10% or more, relative to the baseline total cholesterol measurement. In some embodiments, the total cholesterol measurement is decreased by about 20% or more, about 30% or more, about 40% or more, about 50% or more, about 60% or more, about 70% or more, about 80% or more, about 90% or more, or about 100% or more relative to the baseline total cholesterol measurement. In some embodiments, the total cholesterol measurement is decreased by no more than about 2.5%, no more than about 5%, or no more than about 7.5%, relative to the baseline total cholesterol measurement. In some embodiments, the total cholesterol measurement is decreased by no more than about 10%, relative to the baseline total cholesterol measurement. In some embodiments, the total cholesterol measurement is decreased by no more than about 20%, no more than about 30%, no more than about 40%, no more than about 50%, no more than about 60%, no more than about 70%, no more than about 80%, no more than about 90%, or about 100% relative to the baseline total cholesterol measurement. In some embodiments, the total cholesterol measurement is decreased by 2.5%, 5%, 7.5%0, 10%, 20%, 30%, 40%, 50%, 60%, 70%, 80%, 90%, or 100%, or by a range defined by any of the two aforementioned percentages.

In some embodiments, the measurement is a non-HDL cholesterol measurement. In some embodiments, the non-HDL cholesterol measurement is a blood non-HDL cholesterol concentration (for example, units per liter (mg/dL)). In some embodiments, the non-HDL cholesterol measurement is a circulating non-HDL cholesterol measurement. In some embodiments, the non-HDL cholesterol measurement is obtained by an assay such as an immunoassay, a colorimetric assay, or a fluorescence assay.

In some embodiments, the composition reduces the non-HDL cholesterol measurement relative to the baseline non-HDL cholesterol measurement. In some embodiments, the composition reduces circulating non-HDL cholesterol relative to the baseline non-HDL cholesterol measurement. In some embodiments, the reduced non-HDL cholesterol is measured in a second sample obtained from the subject after administering the composition to the subject. In some embodiments, the non-HDL cholesterol measurement is decreased by about 2.5% or more, about 5% or more, or about 7.5% or more, relative to the baseline non-HDL cholesterol measurement. In some embodiments, the non-HDL cholesterol measurement is decreased by about 10% or more, relative to the baseline non-HDL cholesterol measurement. In some embodiments, the non-HDL cholesterol measurement is decreased by about 20% or more, about 30% or more, about 40% or more, about 50% or more, about 60% or more, about 70% or more, about 80% or more, about 90% or more, or about 100% or more relative to the baseline non-HDL cholesterol measurement. In some embodiments, the non-HDL cholesterol measurement is decreased by no more than about 2.5%, no more than about 5%, or no more than about 7.5%, relative to the baseline non-HDL cholesterol measurement. In some embodiments, the non-HDL cholesterol measurement is decreased by no more than about 10%, relative to the baseline non-HDL cholesterol measurement. In some embodiments, the non-HDL cholesterol measurement is decreased by no more than about 20%, no more than about 30%, no more than about 40%, no more than about 50%, no more than about 60%, no more than about 70%, no more than about 80%, no more than about 90%, or about 100% relative to the baseline non-HDL cholesterol measurement. In some embodiments, the non-HDL cholesterol measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an GPAM protein measurement. In some embodiments, the GPAM protein measurement comprises an GPAM protein level. In some embodiments, the GPAM protein level is indicated as a mass or percentage of GPAM protein per sample weight. In some embodiments, the GPAM protein level is indicated as a mass or percentage of GPAM protein per sample volume. In some embodiments, the GPAM protein level is indicated as a mass or percentage of GPAM protein per total protein within the sample. In some embodiments, the GPAM protein measurement is a tissue GPAM protein measurement. In some embodiments, the GPAM protein measurement is obtained by an assay such as an immunoassay, a colorimetric assay, or a fluorescence assay.

In some embodiments, the composition reduces the GPAM protein measurement relative to the baseline GPAM protein measurement.. In some embodiments, the composition reduces tissue GPAM protein levels relative to the baseline GPAM protein measurement. In some embodiments, the reduced GPAM protein levels are measured in a second sample obtained from the subject after administering the composition to the subject. In some embodiments, the GPAM protein measurement is decreased by about 2.5% or more, about 5% or more, or about 7.5% or more, relative to the baseline GPAM protein measurement. In some embodiments, the GPAM protein measurement is decreased by about 10% or more, relative to the baseline GPAM protein measurement. In some embodiments, the GPAM protein measurement is decreased by about 20% or more, about 30% or more, about 40% or more, about 50% or more, about 60% or more, about 70% or more, about 80% or more, about 90% or more, or about 100%, relative to the baseline GPAM protein measurement. In some embodiments, the GPAM protein measurement is decreased by no more than about 2.5%, no more than about 5%, or no more than about 7.5%, relative to the baseline GPAM protein measurement. In some embodiments, the GPAM protein measurement is decreased by no more than about 10%, relative to the baseline GPAM protein measurement. In some embodiments, the GPAM protein measurement is decreased by no more than about 20%, no more than about 30%, no more than about 40%, no more than about 50%, no more than about 60%, no more than about 70%, no more than about 80%, no more than about 90%, or no more than about 100% relative to the baseline GPAM protein measurement. In some embodiments, the GPAM protein measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is an GPAM mRNA measurement. In some embodiments, the GPAM mRNA measurement comprises an GPAM mRNA level. In some embodiments, the GPAM mRNA level is indicated as an amount or percentage of GPAM mRNA per sample weight. In some embodiments, the GPAM mRNA level is indicated as an amount or percentage of GPAM mRNA per sample volume. In some embodiments, the GPAM mRNA level is indicated as an amount or percentage of GPAM mRNA per total mRNA within the sample. In some embodiments, the GPAM mRNA level is indicated as an amount or percentage of GPAM mRNA per total nucleic acids within the sample. In some embodiments, the GPAM mRNA level is indicated relative to another mRNA level, such as an mRNA level of a housekeeping gene, within the sample. In some embodiments, the GPAM mRNA measurement is a tissue GPAM mRNA measurement. In some embodiments, the GPAM mRNA measurement is obtained by an assay such as a PCR assay. In some embodiments, the PCR comprises qPCR. In some embodiments, the PCR comprises reverse transcription of the GPAM mRNA.

In some embodiments, the composition reduces the GPAM mRNA measurement relative to the baseline GPAM mRNA measurement. In some embodiments, the GPAM mRNA measurement is obtained in a second sample obtained from the subject after administering the composition to the subject. In some embodiments, the composition reduces GPAM mRNA levels relative to the baseline GPAM mRNA levels. In some embodiments, the reduced GPAM mRNA levels are measured in a second sample obtained from the subject after administering the composition to the subject. In some embodiments, the second sample is a liver sample. In some embodiments, the second sample is an adipose sample. In some embodiments, the GPAM mRNA measurement is reduced by about 2.5% or more, about 5% or more, or about 7.50% or more, relative to the baseline GPAM mRNA measurement. In some embodiments, the GPAM mRNA measurement is decreased by about 10% or more, relative to the baseline GPAM mRNA measurement. In some embodiments, the GPAM mRNA measurement is decreased by about 20% or more, about 30% or more, about 40% or more, about 50% or more, about 60% or more, about 70% or more, about 80% or more, about 90% or more, or about 100%, relative to the baseline GPAM mRNA measurement. In some embodiments, the GPAM mRNA measurement is decreased by no more than about 2.5%, no more than about 5%, or no more than about 7.5%, relative to the baseline GPAM mRNA measurement. In some embodiments, the GPAM mRNA measurement is decreased by no more than about 10%, relative to the baseline GPAM mRNA measurement. In some embodiments, the GPAM mRNA measurement is decreased by no more than about 20%, no more than about 30%, no more than about 40%, no more than about 50%, no more than about 60%, no more than about 70%, no more than about 80%, no more than about 90%, or no more than about 100%, relative to the baseline GPAM mRNA measurement. In some embodiments, the GPAM mRNA measurement is decreased by 2.5%, 5%, 7.5%, 10%, 20%, 30%, 40%, 50%, 60%, 70%, 80%, 90%, or 100% or by a range defined by any of the two aforementioned percentages.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “Cx-y” or “Cx-Cy” when used in conjunction with a chemical moiety, such as alkyl, alkenyl, or alkynyl is meant to include groups that contain from x to y carbons in the chain. For example, the term “C1-6alkyl” refers to substituted or unsubstituted saturated hydrocarbon groups, including straight-chain alkyl and branched-chain alkyl groups that contain from 1 to 6 carbons.

The terms “Cx-yalkenyl” and “Cx-yalkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2 trifluoroethyl, 1 chloromethyl 2 fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3 benzodioxolyl, benzofuranyl, benzoxazolyl, benzo[d]thiazolyl, benzothiadiazolyl, benzo[b][1,4]dioxepinyl, benzo[b][1,4]oxazinyl, 1,4 benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2 d]pyrimidinyl, benzotriazolyl, benzo[4,6]imidazo[1,2 a]pyridinyl, carbazolyl, cinnolinyl, cyclopenta[d]pyrimidinyl, 6,7 dihydro 5H cyclopenta[4,5]thieno[2,3 d]pyrimidinyl, 5,6 dihydrobenzo[h]quinazolinyl, 5,6 dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2 c]pyridinyl, 5,6,7,8,9,10 hexahydrocycloocta[d]pyrimidinyl, 5,6,7,8,9,10 hexahydrocycloocta[d]pyridazinyl, 5,6,7,8,9,10 hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8 methano 5,6,7,8 tetrahydroquinazolinyl, naphthyridinyl, 1,6 naphthyridinonyl, oxadiazolyl, 2 oxoazepinyl, oxazolyl, oxiranyl, 5,6,6a, 7,8,9,10,10a octahydrobenzo[h]quinazolinyl, 1 phenyl 1H pyrrolyl, phenazinyl, phenothiazinyl, phenoxazinyl, phthalazinyl, pteridinyl, purinyl, pyrrolyl, pyrazolyl, pyrazolo[3,4 d]pyrimidinyl, pyridinyl, pyrido[3,2 d]pyrimidinyl, pyrido[3,4 d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8 tetrahydroquinazolinyl, 5,6,7,8 tetrahydrobenzo[4,5]thieno[2,3 d]pyrimidinyl, 6,7,8,9 tetrahydro 5H cyclohepta[4,5]thieno[2,3 d]pyrimidinyl, 5,6,7,8 tetrahydropyrido[4,5 c]pyridazinyl, thiazolyl, thiadiazolyl, triazolyl, tetrazolyl, triazinyl, thieno[2,3 d]pyrimidinyl, thieno[3,2 d]pyrimidinyl, thieno[2,3 c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, 2-oxa-6-azaspiro[3.3]heptane, and 1,1 dioxo thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH2 of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO2), imino (═N—H), oximo (═N—OH), hydrazino (═N—NH2), —Rb ORa, —Rb OC(O) Ra, —Rb OC(O) ORa, —Rb OC(O) N(Ra)2, —Rb N(Ra)2, —Rb C(O)Ra, —Rb C(O)ORa, —Rb C(O)N(Ra)2, —Rb O Re C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2), and —Rb S(O)tN(Ra)2 (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO2), imino (═N—H), oximo (═N—OH), hydrazine (═N—NH2), —Rb ORa, —Rb OC(O) Ra, —Rb OC(O) ORa, —Rb OC(O) N(Ra)₂, —Rb N(Ra)₂, —Rb C(O)Ra, —Rb C(O)ORa, —Rb C(O)N(Ra)₂, —Rb O Rc C(O)N(Ra)₂, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)2 (where t is 1 or 2); wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each Ra, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO2), imino (═N—H), oximo (═N—OH), hydrazine (═N—NH2), —Rb ORa, —Rb OC(O) Ra, —Rb OC(O) ORa, —Rb OC(O) N(Ra)₂, —Rb N(Ra)₂, —Rb C(O)Ra, —Rb C(O)ORa, —Rb C(O)N(Ra)₂, —Rb O Rc C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)2 (where t is 1 or 2); and wherein each Rb is independently selected from a direct bond or a straight or branched alkylene, alkenylene, or alkynylene chain, and each Rc is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

In some embodiments, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

Some embodiments refer to nucleic acid sequence information. It is contemplated that in some embodiments, thymine (T) may be interchanged with uracil (U), or vice versa. For example, some sequences in the sequence listing may recite Ts, but these may be replaced with Us in some embodiments. In some oligonucleotides with nucleic acid sequences that include uracil, the uracil may be replaced with thymine. Similarly, in some oligonucleotides with nucleic acid sequences that include thymine, the thymine may be replaced with uracil. In some embodiments, an oligonucleotide such as an siRNA comprises or consists of RNA. In some embodiments, the oligonucleotide may include DNA. For example, the oligonucleotide may include 2′ deoxyribonucleotides. An ASO may comprise or consist of DNA. To any extent that the sequence listing contradicts the disclosure in the specification, the specification takes precedence.

Some aspects include sequences with nucleotide modifications or modified internucleoside linkages. Generally, and unless otherwise specified, Nf (e.g. Af, Cf, Gf, Tf, or Uf) refers to a 2′ fluoro-modified nucleoside, dN (e.g. dA, dC, dG, dT, or dU) refers to a 2′ deoxy nucleoside, n (e.g. a, c, g, t, or u) refers to a 2′ O-methyl modified nucleoside, and “s” refers to a phosphorothioate linkage.

A pyrimidine may include cytosine (C), thymine (T), or uracil (U). A pyrimidine may include C or U. A pyrimidine may include C or T. A reference to a pyrimidine may include a nucleoside or nucleotide comprising the pyrimidine. A purine may include guanine (G) or adenine (A). A reference to a purine may include a nucleoside or nucleotide comprising a purine.

The section headings used herein are for organizational purposes only and are not to be construed as limiting the subject matter described.

### VI. EXAMPLES

### Example 1: Functional Variants in GPAM Demonstrate Protective Associations for Non-Alcoholic Fatty Liver Disease, Alcoholic Liver Disease, Decreased Liver Fat Percentage and Decreased Blood Lipids

Variants in GPAM were evaluated for associations with liver disease, liver fat percentage, liver function parameters, and blood lipid levels in approximately 452,000 individuals from the UK Biobank cohort. Variants evaluated included rs2792751, a common (AAF=0.73) missense variant (I43V); and rs57128949 a common (AAF=0.18) intergenic variant that is a GPAM mRNA expression quantitative trait locus (eQTL) associated with decreased GPAM mRNA expression in multiple tissues, including liver. Stepwise conditional analyses in multiple traits, as well as direct evaluation of linkage disequilibrium, confirmed that rs2792751 and rs57128949 are independent variants. Some details of these variants are shown in Table 3.

TABLE 3
GPAM variants

| Variant | Consequence | AAF |
|---|---|---|
| rs2792751 | missense (I43V) | 0.73 |
| rs57128949 | eQTL | 0.18 |

The analyses resulted in identification of associations for the individual GPAM variants and phenotypes. Applicant hypothesizes that individually these variants result in a decrease in the abundance and/or activity of the GPAM gene product, and that it is this loss of function that leads to the observed genetic associations. This hypothesis is supported by associations between the GPAM variants and increased levels of blood ketone bodies as measured by nuclear magnetic resonance in the UK Biobank, including 3-hydroxybutyrate, acetoacetate and acetone (Table 4). These results were consistent with observations in GPAM knockout mice, which demonstrate increased plasma ketone bodies, and with the known function of GPAM in triacylglycerol (TAG) metabolism.

TABLE 4
GPAM variant blood ketone associations

| | 3-Hydroxybutyrate | | Acetoacetate | | Acetone | |
|---|---|---|---|---|---|---|
| | (n = 108,920) | | (n = 110,462) | | (n = 110,671) | |
| Variant | P value | Beta | P value | Beta | P value | Beta |
| rs2792751 | 3.03E−22 | ↑0.046 | 1.27E−07 | ↑0.025 | 1.05E−06 | ↑0.023 |
| rs57128949 | 2.80E−04 | ↑0.020 | 7.76E−04 | ↑0.019 | 0.265 | ↑0.006 |

The analyses resulted in identification of associations for the GPAM variants. For example, there were protective associations with multiple liver disease-related traits. GPAM variants are individually associated with decreased risk of non-alcoholic fatty liver disease (NAFLD), alcoholic liver disease, liver fibrosis and cirrhosis, decreased risk of sequelae of chronic liver disease such as esophageal varices and portal hypertension, and with decreased Mm-derived liver fat percentage (Table 5). GPAM variants are also associated with decreased blood levels of liver enzymes, including alanine aminotransferase (ALT), aspartate aminotransferase (AST) and alkaline phosphatase (ALP), and with decreased blood bilirubin (Table 6).

TABLE 5
GPAM variant liver disease-related associations

| | Nonalcoholic Fatty Liver | | MRI Liver Fat | | Alcoholic Liver | |
|---|---|---|---|---|---|---|
| | Disease (NAFLD) | | Percentage | | Disease | |
| | (n = 7,792) | | (n = 9,011) | | (n = 1,915) | |
| Variant | P value | OR | P value | Beta | P value | OR |
| rs2792751 | 1.14E−10 | ↓0.869 | 1.05E−05 | ↓−0.024 | 3.09E−03 | ↓0.908 |
| rs57128949 | 4.30E−03 | ↓0.927 | 0.015 | ↓−0.015 | 0.066 | ↓0.933 |
| | Liver Fibrosis and | | Esophageal Varices | | Portal Hypertension | |
| | Cirrhosis (n = 2,298) | | (n = 1,216) | | (n = 1,249) | |
| Variant | P value | OR | P value | OR | P value | OR |
| rs2792751 | 3.22E−03 | ↓0.915 | 5.94E−04 | ↓0.860 | 0.026 | ↓0.919 |
| rs57128949 | 0.156 | ↓0.948 | 3.45E−03 | ↓0.854 | 0.048 | ↓0.902 |

TABLE 6
GPAM variant liver function associations

| | Alanine | | Aspartate | | Alkaline | | | |
|---|---|---|---|---|---|---|---|---|
| | Aminotransferase | | Aminotransferase | | Phosphatase | | Bilirubin | |
| | (n = 430,545) | | (n = 429,246) | | (n = 431,716) | | (n = 428,967) | |
| Variant | P value | Beta | P value | Beta | P value | Beta | P value | Beta |
| rs2792751 | 3.37E−28 | ↓−0.005 | 1.65E−10 | ↓−0.002 | 1.48E−46 | ↓−0.004 | 7.87E−22 | ↓−0.003 |
| rs57128949 | 1.94E−16 | ↓−0.004 | 2.17E−06 | ↓−0.001 | 4.79E−23 | ↓−0.003 | 6.40E−08 | ↓−0.002 |

GPAM variants are also associated with decreased LDL-cholesterol, total cholesterol and APOB, and with decreased use of ‘statin’ (HMG CoA reductase inhibitor) medications (Table 7). These protective associations with putative loss of function variants in GPAM across several related and distinct diseases and traits suggest that inhibition of GPAM could be therapeutic in these and related diseases.

TABLE 7
GPAM variant blood lipid associations

| | LDL Cholesterol | | Total Cholesterol | |
|---|---|---|---|---|
| | (n = 431,767) | | (n = 432,322) | |
| Variant | P value | Beta | P value | Beta |
| rs2792751 | 1.09E−24 | ↓−0.003 | 1.11E−35 | ↓−0.002 |
| rs57128949 | 1.85E−08 | ↓−0.002 | 9.76E−13 | ↓−0.002 |

| | APOB | | Statin Medication | |
|---|---|---|---|---|
| | (n = 430,494) | | (n = 104,279) | |
| Variant | P value | Beta | P value | OR |
| rs2792751 | 7.30E−05 | ↓−0.001 | 4.18E−05 | ↓0.978 |
| rs57128949 | 0.085 | ↓−4.78E−04 | 0.024 | ↓0.992 |

### Example 2: Bioinformatic Selection of Sequences in Order to Identify Therapeutic siRNAs to Downmodulate Expression of the GPAM mRNA

Screening sets were defined based on bioinformatic analysis. Therapeutic siRNAs were designed to target human GPAM. Predicted specificity in human, rhesus monkey, cynomolgus monkey, mouse, rat, rabbit, and dog was determined for sense (S) and antisense (AS) strands. These were assigned a “specificity score” which considers the likelihood of unintended downregulation of any other transcript by full or partial complementarity of an siRNA strand (up to 2 mismatches within positions 2-18) as well as the number and positions of mismatches. Thus, off-target(s) transcripts for antisense and sense strands of each siRNA were identified. As identified, siRNAs with high specificity and a low number of predicted off-targets provided a benefit of increased targeting specificity.

In addition to selecting siRNA sequences with high sequence specificity to GPAM mRNA, siRNA sequences within the seed region were analyzed for similarity to seed regions of known miRNAs. siRNAs can function in a miRNA like manner via base-pairing with complementary sequences within the 3′-UTR of mRNA molecules. The complementarity typically encompasses the 5′-bases at positions 2-7 of the miRNA (seed region). To circumvent siRNAs to act via functional miRNA binding sites, siRNA strands containing natural miRNA seed regions can be avoided. Seed regions identified in miRNAs from human, mouse, rat, rhesus monkey, dog, rabbit, and pig are referred to as “conserved”. Combining the “specificity score” with miRNA seed analysis yielded a “specificity category”. This is divided into categories 1-4, with 1 having the highest specificity and 4 having the lowest specificity. Each strand of the siRNA is assigned to a specificity category.

Analysis of the Genome Aggregation Database (gnomAD) to identify siRNAs targeting regions with known SNPs was also carried out to identify siRNAs that may be non-functional in individuals containing the SNP. Information regarding the positions of SNPs within the target sequence as well as minor allele frequency (MAF) in case data was obtained in this analysis.

Initial analysis of the relevant GPAM mRNA sequence revealed few sequences that fulfil the specificity parameters and at the same time target GPAM mRNA in all the analyzed relevant species. Therefore, independent screening subsets were designed for the therapeutic siRNAs.

The siRNAs in these subsets recognized at least the human GPAM sequences. Therefore, the siRNAs in these subsets can be used to target human GPAM in a therapeutic setting.

The number of siRNA sequences derived from human GPAM mRNA (ENST00000348367.9, SEQ ID NO: 12867) without consideration of specificity or species cross-reactivity was 6354 (sense and antisense strand sequences included in SEQ ID NOS: 1-6354 and 6355-12708, respectively).

Prioritizing sequences for target specificity, miRNA seed region sequences and SNPs as described above yields subset A. Subset A contains 1348 siRNAs whose base sequences are shown in Table 8.

The above methods can be used to identify therapeutic siRNAs to downmodulate expression of the GPAM mRNA.

TABLE 8
Sequences in siRNA subset A

| | SEQ | | SEQ | |
|---|---|---|---|---|
| siRNA | ID | Sense strand | ID | Antisense strand |
| Name | NO: | sequence (5′-3′) | NO: | sequence (5′-3′) |
| siRNA 13 | 13 | CUGGCAUUGGCCGGGACUG | 6367 | CAGUCCCGGCCAAUGCCAG |
| siRNA 18 | 18 | AUUGGCCGGGACUGGAAGU | 6372 | ACUUCCAGUCCCGGCCAAU |
| siRNA 23 | 23 | CCGGGACUGGAAGUGCGGG | 6377 | CCCGCACUUCCAGUCCCGG |
| siRNA 26 | 26 | GGACUGGAAGUGCGGGCUU | 6380 | AAGCCCGCACUUCCAGUCC |
| siRNA 28 | 28 | ACUGGAAGUGCGGGCUUCU | 6382 | AGAAGCCCGCACUUCCAGU |
| siRNA 54 | 54 | CCGAAGCUGGAGCUGCUAG | 6408 | CUAGCAGCUCCAGCUUCGG |
| siRNA 62 | 62 | GGAGCUGCUAGGGCAGCAG | 6416 | CUGCUGCCCUAGCAGCUCC |
| siRNA 79 | 79 | AGCGGCUCCCCUGUUGUAU | 6433 | AUACAACAGGGGAGCCGCU |
| siRNA 81 | 81 | CGGCUCCCCUGUUGUAUGG | 6435 | CCAUACAACAGGGGAGCCG |
| siRNA 82 | 82 | GGCUCCCCUGUUGUAUGGA | 6436 | UCCAUACAACAGGGGAGCC |
| siRNA 83 | 83 | GCUCCCCUGUUGUAUGGAC | 6437 | GUCCAUACAACAGGGGAGC |
| siRNA 84 | 84 | CUCCCCUGUUGUAUGGACA | 6438 | UGUCCAUACAACAGGGGAG |
| siRNA 88 | 88 | CCUGUUGUAUGGACAUUCU | 6442 | AGAAUGUCCAUACAACAGG |
| siRNA 90 | 90 | UGUUGUAUGGACAUUCUGC | 6444 | GCAGAAUGUCCAUACAACA |
| siRNA 91 | 91 | GUUGUAUGGACAUUCUGCA | 6445 | UGCAGAAUGUCCAUACAAC |
| siRNA 94 | 94 | GUAUGGACAUUCUGCACCC | 6448 | GGGUGCAGAAUGUCCAUAC |
| siRNA 95 | 95 | UAUGGACAUUCUGCACCCG | 6449 | CGGGUGCAGAAUGUCCAUA |
| siRNA 96 | 96 | AUGGACAUUCUGCACCCGA | 6450 | UCGGGUGCAGAAUGUCCAU |
| siRNA 98 | 98 | GGACAUUCUGCACCCGAAA | 6452 | UUUCGGGUGCAGAAUGUCC |
| siRNA 100 | 100 | ACAUUCUGCACCCGAAACU | 6454 | AGUUUCGGGUGCAGAAUGU |
| siRNA 101 | 101 | CAUUCUGCACCCGAAACUG | 6455 | CAGUUUCGGGUGCAGAAUG |
| siRNA 106 | 106 | UGCACCCGAAACUGAUAGC | 6460 | GCUAUCAGUUUCGGGUGCA |
| siRNA 107 | 107 | GCACCCGAAACUGAUAGCU | 6461 | AGCUAUCAGUUUCGGGUGC |
| siRNA 108 | 108 | CACCCGAAACUGAUAGCUG | 6462 | CAGCUAUCAGUUUCGGGUG |
| siRNA 109 | 109 | ACCCGAAACUGAUAGCUGA | 6463 | UCAGCUAUCAGUUUCGGGU |
| siRNA 110 | 110 | CCCGAAACUGAUAGCUGAG | 6464 | CUCAGCUAUCAGUUUCGGG |
| siRNA 111 | 111 | CCGAAACUGAUAGCUGAGU | 6465 | ACUCAGCUAUCAGUUUCGG |
| siRNA 113 | 113 | GAAACUGAUAGCUGAGUCC | 6467 | GGACUCAGCUAUCAGUUUC |
| siRNA 114 | 114 | AAACUGAUAGCUGAGUCCU | 6468 | AGGACUCAGCUAUCAGUUU |
| siRNA 118 | 118 | UGAUAGCUGAGUCCUGAAG | 6472 | CUUCAGGACUCAGCUAUCA |
| siRNA 119 | 119 | GAUAGCUGAGUCCUGAAGU | 6473 | ACUUCAGGACUCAGCUAUC |
| siRNA 127 | 127 | AGUCCUGAAGUUUUAUGUU | 6481 | AACAUAAAACUUCAGGACU |
| siRNA 161 | 161 | UUUCAUCCCAGCACAUGAU | 6515 | AUCAUGUGCUGGGAUGAAA |
| siRNA 162 | 162 | UUCAUCCCAGCACAUGAUU | 6516 | AAUCAUGUGCUGGGAUGAA |
| siRNA 170 | 170 | AGCACAUGAUUUGGGAAUU | 6524 | AAUUCCCAAAUCAUGUGCU |
| siRNA 173 | 173 | ACAUGAUUUGGGAAUUACA | 6527 | UGUAAUUCCCAAAUCAUGU |
| siRNA 175 | 175 | AUGAUUUGGGAAUUACACU | 6529 | AGUGUAAUUCCCAAAUCAU |
| siRNA 176 | 176 | UGAUUUGGGAAUUACACUU | 6530 | AAGUGUAAUUCCCAAAUCA |
| siRNA 177 | 177 | GAUUUGGGAAUUACACUUU | 6531 | AAAGUGUAAUUCCCAAAUC |
| siRNA 179 | 179 | UUUGGGAAUUACACUUUGU | 6533 | ACAAAGUGUAAUUCCCAAA |
| siRNA 181 | 181 | UGGGAAUUACACUUUGUGA | 6535 | UCACAAAGUGUAAUUCCCA |
| siRNA 190 | 190 | CACUUUGUGACAUGGAUGA | 6544 | UCAUCCAUGUCACAAAGUG |
| siRNA 191 | 191 | ACUUUGUGACAUGGAUGAA | 6545 | UUCAUCCAUGUCACAAAGU |
| siRNA 194 | 194 | UUGUGACAUGGAUGAAUCU | 6548 | AGAUUCAUCCAUGUCACAA |
| siRNA 202 | 202 | UGGAUGAAUCUGCACUGAC | 6556 | GUCAGUGCAGAUUCAUCCA |
| siRNA 206 | 206 | UGAAUCUGCACUGACCCUU | 6560 | AAGGGUCAGUGCAGAUUCA |
| siRNA 207 | 207 | GAAUCUGCACUGACCCUUG | 6561 | CAAGGGUCAGUGCAGAUUC |
| siRNA 211 | 211 | CUGCACUGACCCUUGGUAC | 6565 | GUACCAAGGGUCAGUGCAG |
| siRNA 216 | 216 | CUGACCCUUGGUACAAUAG | 6570 | CUAUUGUACCAAGGGUCAG |
| siRNA 218 | 218 | GACCCUUGGUACAAUAGAU | 6572 | AUCUAUUGUACCAAGGGUC |
| siRNA 221 | 221 | CCUUGGUACAAUAGAUGUU | 6575 | AACAUCUAUUGUACCAAGG |
| siRNA 232 | 232 | UAGAUGUUUCUUAUCUGCC | 6586 | GGCAGAUAAGAAACAUCUA |
| siRNA 241 | 241 | CUUAUCUGCCACAUUCAUC | 6595 | GAUGAAUGUGGCAGAUAAG |
| siRNA 246 | 246 | CUGCCACAUUCAUCAGAAU | 6600 | AUUCUGAUGAAUGUGGCAG |
| siRNA 247 | 247 | UGCCACAUUCAUCAGAAUA | 6601 | UAUUCUGAUGAAUGUGGCA |
| siRNA 257 | 257 | AUCAGAAUACAGUGUUGGU | 6611 | ACCAACACUGUAUUCUGAU |
| siRNA 260 | 260 | AGAAUACAGUGUUGGUCGA | 6614 | UCGACCAACACUGUAUUCU |
| siRNA 261 | 261 | GAAUACAGUGUUGGUCGAU | 6615 | AUCGACCAACACUGUAUUC |
| siRNA 262 | 262 | AAUACAGUGUUGGUCGAUG | 6616 | CAUCGACCAACACUGUAUU |
| siRNA 263 | 263 | AUACAGUGUUGGUCGAUGU | 6617 | ACAUCGACCAACACUGUAU |
| siRNA 264 | 264 | UACAGUGUUGGUCGAUGUA | 6618 | UACAUCGACCAACACUGUA |
| siRNA 265 | 265 | ACAGUGUUGGUCGAUGUAA | 6619 | UUACAUCGACCAACACUGU |
| siRNA 267 | 267 | AGUGUUGGUCGAUGUAAGC | 6621 | GCUUACAUCGACCAACACU |
| siRNA 275 | 275 | UCGAUGUAAGCACACAAGU | 6629 | ACUUGUGUGCUUACAUCGA |
| siRNA 278 | 278 | AUGUAAGCACACAAGUGAG | 6632 | CUCACUUGUGUGCUUACAU |
| siRNA 279 | 279 | UGUAAGCACACAAGUGAGG | 6633 | CCUCACUUGUGUGCUUACA |
| siRNA 280 | 280 | GUAAGCACACAAGUGAGGA | 6634 | UCCUCACUUGUGUGCUUAC |
| siRNA 284 | 284 | GCACACAAGUGAGGAAUGG | 6638 | CCAUUCCUCACUUGUGUGC |
| siRNA 303 | 303 | GGUGAGUGUGGCUUUAGAC | 6657 | GUCUAAAGCCACACUCACC |
| siRNA 331 | 331 | UCAGAUCUGCAACUUUAAA | 6685 | UUUAAAGUUGCAGAUCUGA |
| siRNA 354 | 354 | AAAGAAAGCCUAAUGAGUC | 6708 | GACUCAUUAGGCUUUCUUU |
| siRNA 356 | 356 | AGAAAGCCUAAUGAGUCGG | 6710 | CCGACUCAUUAGGCUUUCU |
| siRNA 357 | 357 | GAAAGCCUAAUGAGUCGGA | 6711 | UCCGACUCAUUAGGCUUUC |
| siRNA 358 | 358 | AAAGCCUAAUGAGUCGGAA | 6712 | UUCCGACUCAUUAGGCUUU |
| siRNA 359 | 359 | AAGCCUAAUGAGUCGGAAA | 6713 | UUUCCGACUCAUUAGGCUU |
| siRNA 360 | 360 | AGCCUAAUGAGUCGGAAAA | 6714 | UUUUCCGACUCAUUAGGCU |
| siRNA 361 | 361 | GCCUAAUGAGUCGGAAAAG | 6715 | CUUUUCCGACUCAUUAGGC |
| siRNA 362 | 362 | CCUAAUGAGUCGGAAAAGG | 6716 | CCUUUUCCGACUCAUUAGG |
| siRNA 363 | 363 | CUAAUGAGUCGGAAAAGGC | 6717 | GCCUUUUCCGACUCAUUAG |
| siRNA 364 | 364 | UAAUGAGUCGGAAAAGGCC | 6718 | GGCCUUUUCCGACUCAUUA |
| siRNA 365 | 365 | AAUGAGUCGGAAAAGGCCA | 6719 | UGGCCUUUUCCGACUCAUU |
| siRNA 370 | 370 | GUCGGAAAAGGCCAUUUGU | 6724 | ACAAAUGGCCUUUUCCGAC |
| siRNA 386 | 386 | UGUUGGAAGAUGUUGUUAC | 6740 | GUAACAACAUCUUCCAACA |
| siRNA 397 | 397 | GUUGUUACUCCUGCACUCC | 6751 | GGAGUGCAGGAGUAACAAC |
| siRNA 401 | 401 | UUACUCCUGCACUCCCCAG | 6755 | CUGGGGAGUGCAGGAGUAA |
| siRNA 403 | 403 | ACUCCUGCACUCCCCAGAG | 6757 | CUCUGGGGAGUGCAGGAGU |
| siRNA 417 | 417 | CAGAGCUGGGACAAAUUUU | 6771 | AAAAUUUGUCCCAGCUCUG |
| siRNA 425 | 425 | GGACAAAUUUUUCAACCCC | 6779 | GGGGUUGAAAAAUUUGUCC |
| siRNA 427 | 427 | ACAAAUUUUUCAACCCCAG | 6781 | CUGGGGUUGAAAAAUUUGU |
| siRNA 432 | 432 | UUUUUCAACCCCAGUAUCC | 6786 | GGAUACUGGGGUUGAAAAA |
| siRNA 435 | 435 | UUCAACCCCAGUAUCCCGU | 6789 | ACGGGAUACUGGGGUUGAA |
| siRNA 436 | 436 | UCAACCCCAGUAUCCCGUC | 6790 | GACGGGAUACUGGGGUUGA |
| siRNA 444 | 444 | AGUAUCCCGUCUUUGGGUU | 6798 | AACCCAAAGACGGGAUACU |
| siRNA 450 | 450 | CCGUCUUUGGGUUUGCGGA | 6804 | UCCGCAAACCCAAAGACGG |
| siRNA 454 | 454 | CUUUGGGUUUGCGGAAUGU | 6808 | ACAUUCCGCAAACCCAAAG |
| siRNA 457 | 457 | UGGGUUUGCGGAAUGUUAU | 6811 | AUAACAUUCCGCAAACCCA |
| siRNA 458 | 458 | GGGUUUGCGGAAUGUUAUU | 6812 | AAUAACAUUCCGCAAACCC |
| siRNA 487 | 487 | AAACUCACACAAGACACCG | 6841 | CGGUGUCUUGUGUGAGUUU |
| siRNA 493 | 493 | ACACAAGACACCGCGGAUG | 6847 | CAUCCGCGGUGUCUUGUGU |
| siRNA 494 | 494 | CACAAGACACCGCGGAUGG | 6848 | CCAUCCGCGGUGUCUUGUG |
| siRNA 495 | 495 | ACAAGACACCGCGGAUGGC | 6849 | GCCAUCCGCGGUGUCUUGU |
| siRNA 496 | 496 | CAAGACACCGCGGAUGGCU | 6850 | AGCCAUCCGCGGUGUCUUG |
| siRNA 497 | 497 | AAGACACCGCGGAUGGCUU | 6851 | AAGCCAUCCGCGGUGUCUU |
| siRNA 498 | 498 | AGACACCGCGGAUGGCUUG | 6852 | CAAGCCAUCCGCGGUGUCU |
| siRNA 499 | 499 | GACACCGCGGAUGGCUUGC | 6853 | GCAAGCCAUCCGCGGUGUC |
| siRNA 501 | 501 | CACCGCGGAUGGCUUGCAA | 6855 | UUGCAAGCCAUCCGCGGUG |
| siRNA 506 | 506 | CGGAUGGCUUGCAAGACGC | 6860 | GCGUCUUGCAAGCCAUCCG |
| siRNA 507 | 507 | GGAUGGCUUGCAAGACGCC | 6861 | GGCGUCUUGCAAGCCAUCC |
| siRNA 510 | 510 | UGGCUUGCAAGACGCCUUU | 6864 | AAAGGCGUCUUGCAAGCCA |
| siRNA 511 | 511 | GGCUUGCAAGACGCCUUUC | 6865 | GAAAGGCGUCUUGCAAGCC |
| siRNA 515 | 515 | UGCAAGACGCCUUUCUUAC | 6869 | GUAAGAAAGGCGUCUUGCA |
| siRNA 517 | 517 | CAAGACGCCUUUCUUACGU | 6871 | ACGUAAGAAAGGCGUCUUG |
| siRNA 518 | 518 | AAGACGCCUUUCUUACGUU | 6872 | AACGUAAGAAAGGCGUCUU |
| siRNA 519 | 519 | AGACGCCUUUCUUACGUUC | 6873 | GAACGUAAGAAAGGCGUCU |
| siRNA 520 | 520 | GACGCCUUUCUUACGUUCU | 6874 | AGAACGUAAGAAAGGCGUC |
| siRNA 521 | 521 | ACGCCUUUCUUACGUUCUU | 6875 | AAGAACGUAAGAAAGGCGU |
| siRNA 522 | 522 | CGCCUUUCUUACGUUCUUU | 6876 | AAAGAACGUAAGAAAGGCG |
| siRNA 532 | 532 | ACGUUCUUUUUAUUCAAGA | 6886 | UCUUGAAUAAAAAGAACGU |
| siRNA 536 | 536 | UCUUUUUAUUCAAGAGCGA | 6890 | UCGCUCUUGAAUAAAAAGA |
| siRNA 537 | 537 | CUUUUUAUUCAAGAGCGAG | 6891 | CUCGCUCUUGAAUAAAAAG |
| siRNA 542 | 542 | UAUUCAAGAGCGAGAUGUG | 6896 | CACAUCUCGCUCUUGAAUA |
| siRNA 543 | 543 | AUUCAAGAGCGAGAUGUGC | 6897 | GCACAUCUCGCUCUUGAAU |
| siRNA 544 | 544 | UUCAAGAGCGAGAUGUGCA | 6898 | UGCACAUCUCGCUCUUGAA |
| siRNA 547 | 547 | AAGAGCGAGAUGUGCAUAA | 6901 | UUAUGCACAUCUCGCUCUU |
| siRNA 548 | 548 | AGAGCGAGAUGUGCAUAAG | 6902 | CUUAUGCACAUCUCGCUCU |
| siRNA 549 | 549 | GAGCGAGAUGUGCAUAAGG | 6903 | CCUUAUGCACAUCUCGCUC |
| siRNA 550 | 550 | AGCGAGAUGUGCAUAAGGG | 6904 | CCCUUAUGCACAUCUCGCU |
| siRNA 551 | 551 | GCGAGAUGUGCAUAAGGGC | 6905 | GCCCUUAUGCACAUCUCGC |
| siRNA 554 | 554 | AGAUGUGCAUAAGGGCAUG | 6908 | CAUGCCCUUAUGCACAUCU |
| siRNA 562 | 562 | AUAAGGGCAUGUUUGCCAC | 6916 | GUGGCAAACAUGCCCUUAU |
| siRNA 564 | 564 | AAGGGCAUGUUUGCCACCA | 6918 | UGGUGGCAAACAUGCCCUU |
| siRNA 565 | 565 | AGGGCAUGUUUGCCACCAA | 6919 | UUGGUGGCAAACAUGCCCU |
| siRNA 596 | 596 | UGUGCUGAACAGCAGUAGA | 6950 | UCUACUGCUGUUCAGCACA |
| siRNA 598 | 598 | UGCUGAACAGCAGUAGAGU | 6952 | ACUCUACUGCUGUUCAGCA |
| siRNA 601 | 601 | UGAACAGCAGUAGAGUACA | 6955 | UGUACUCUACUGCUGUUCA |
| siRNA 603 | 603 | AACAGCAGUAGAGUACAAG | 6957 | CUUGUACUCUACUGCUGUU |
| siRNA 613 | 613 | GAGUACAAGAGGCAAUUGC | 6967 | GCAAUUGCCUCUUGUACUC |
| siRNA 643 | 643 | CUGAAUUAAACCCUGAUGG | 6997 | CCAUCAGGGUUUAAUUCAG |
| siRNA 644 | 644 | UGAAUUAAACCCUGAUGGU | 6998 | ACCAUCAGGGUUUAAUUCA |
| siRNA 646 | 646 | AAUUAAACCCUGAUGGUUC | 7000 | GAACCAUCAGGGUUUAAUU |
| siRNA 647 | 647 | AUUAAACCCUGAUGGUUCU | 7001 | AGAACCAUCAGGGUUUAAU |
| siRNA 664 | 664 | CUGCCCAGCAGCAAUCAAA | 7018 | UUUGAUUGCUGCUGGGCAG |
| siRNA 671 | 671 | GCAGCAAUCAAAAGCCGUU | 7025 | AACGGCUUUUGAUUGCUGC |
| siRNA 672 | 672 | CAGCAAUCAAAAGCCGUUA | 7026 | UAACGGCUUUUGAUUGCUG |
| siRNA 673 | 673 | AGCAAUCAAAAGCCGUUAA | 7027 | UUAACGGCUUUUGAUUGCU |
| siRNA 674 | 674 | GCAAUCAAAAGCCGUUAAC | 7028 | GUUAACGGCUUUUGAUUGC |
| siRNA 675 | 675 | CAAUCAAAAGCCGUUAACA | 7029 | UGUUAACGGCUUUUGAUUG |
| siRNA 676 | 676 | AAUCAAAAGCCGUUAACAA | 7030 | UUGUUAACGGCUUUUGAUU |
| siRNA 679 | 679 | CAAAAGCCGUUAACAAAGU | 7033 | ACUUUGUUAACGGCUUUUG |
| siRNA 681 | 681 | AAAGCCGUUAACAAAGUGA | 7035 | UCACUUUGUUAACGGCUUU |
| siRNA 683 | 683 | AGCCGUUAACAAAGUGAAA | 7037 | UUUCACUUUGUUAACGGCU |
| siRNA 703 | 703 | AGAAAGCUAAAAGGAUUCU | 7057 | AGAAUCCUUUUAGCUUUCU |
| siRNA 723 | 723 | CAAGAAAUGGUUGCCACUG | 7077 | CAGUGGCAACCAUUUCUUG |
| siRNA 730 | 730 | UGGUUGCCACUGUCUCACC | 7084 | GGUGAGACAGUGGCAACCA |
| siRNA 731 | 731 | GGUUGCCACUGUCUCACCG | 7085 | CGGUGAGACAGUGGCAACC |
| siRNA 733 | 733 | UUGCCACUGUCUCACCGGC | 7087 | GCCGGUGAGACAGUGGCAA |
| siRNA 736 | 736 | CCACUGUCUCACCGGCAAU | 7090 | AUUGCCGGUGAGACAGUGG |
| siRNA 737 | 737 | CACUGUCUCACCGGCAAUG | 7091 | CAUUGCCGGUGAGACAGUG |
| siRNA 738 | 738 | ACUGUCUCACCGGCAAUGA | 7092 | UCAUUGCCGGUGAGACAGU |
| siRNA 739 | 739 | CUGUCUCACCGGCAAUGAU | 7093 | AUCAUUGCCGGUGAGACAG |
| siRNA 740 | 740 | UGUCUCACCGGCAAUGAUC | 7094 | GAUCAUUGCCGGUGAGACA |
| siRNA 741 | 741 | GUCUCACCGGCAAUGAUCA | 7095 | UGAUCAUUGCCGGUGAGAC |
| siRNA 742 | 742 | UCUCACCGGCAAUGAUCAG | 7096 | CUGAUCAUUGCCGGUGAGA |
| siRNA 744 | 744 | UCACCGGCAAUGAUCAGAC | 7098 | GUCUGAUCAUUGCCGGUGA |
| siRNA 745 | 745 | CACCGGCAAUGAUCAGACU | 7099 | AGUCUGAUCAUUGCCGGUG |
| siRNA 746 | 746 | ACCGGCAAUGAUCAGACUG | 7100 | CAGUCUGAUCAUUGCCGGU |
| siRNA 748 | 748 | CGGCAAUGAUCAGACUGAC | 7102 | GUCAGUCUGAUCAUUGCCG |
| siRNA 753 | 753 | AUGAUCAGACUGACUGGGU | 7107 | ACCCAGUCAGUCUGAUCAU |
| siRNA 756 | 756 | AUCAGACUGACUGGGUGGG | 7110 | CCCACCCAGUCAGUCUGAU |
| siRNA 760 | 760 | GACUGACUGGGUGGGUGCU | 7114 | AGCACCCACCCAGUCAGUC |
| siRNA 765 | 765 | ACUGGGUGGGUGCUGCUAA | 7119 | UUAGCAGCACCCACCCAGU |
| siRNA 766 | 766 | CUGGGUGGGUGCUGCUAAA | 7120 | UUUAGCAGCACCCACCCAG |
| siRNA 770 | 770 | GUGGGUGCUGCUAAAACUG | 7124 | CAGUUUUAGCAGCACCCAC |
| siRNA 771 | 771 | UGGGUGCUGCUAAAACUGU | 7125 | ACAGUUUUAGCAGCACCCA |
| siRNA 777 | 777 | CUGCUAAAACUGUUCAACA | 7131 | UGUUGAACAGUUUUAGCAG |
| siRNA 781 | 781 | UAAAACUGUUCAACAGCUU | 7135 | AAGCUGUUGAACAGUUUUA |
| siRNA 790 | 790 | UCAACAGCUUCUUUUGGAA | 7144 | UUCCAAAAGAAGCUGUUGA |
| siRNA 809 | 809 | CAUUCAAAUUCACAAAGGU | 7163 | ACCUUUGUGAAUUUGAAUG |
| siRNA 814 | 814 | AAAUUCACAAAGGUCAACU | 7168 | AGUUGACCUUUGUGAAUUU |
| siRNA 819 | 819 | CACAAAGGUCAACUUGAGA | 7173 | UCUCAAGUUGACCUUUGUG |
| siRNA 820 | 820 | ACAAAGGUCAACUUGAGAU | 7174 | AUCUCAAGUUGACCUUUGU |
| siRNA 824 | 824 | AGGUCAACUUGAGAUGGUU | 7178 | AACCAUCUCAAGUUGACCU |
| siRNA 826 | 826 | GUCAACUUGAGAUGGUUAA | 7180 | UUAACCAUCUCAAGUUGAC |
| siRNA 834 | 834 | GAGAUGGUUAAAGCUGCAA | 7188 | UUGCAGCUUUAACCAUCUC |
| siRNA 835 | 835 | AGAUGGUUAAAGCUGCAAC | 7189 | GUUGCAGCUUUAACCAUCU |
| siRNA 839 | 839 | GGUUAAAGCUGCAACUGAG | 7193 | CUCAGUUGCAGCUUUAACC |
| siRNA 843 | 843 | AAAGCUGCAACUGAGACGA | 7197 | UCGUCUCAGUUGCAGCUUU |
| siRNA 845 | 845 | AGCUGCAACUGAGACGAAU | 7199 | AUUCGUCUCAGUUGCAGCU |
| siRNA 846 | 846 | GCUGCAACUGAGACGAAUU | 7200 | AAUUCGUCUCAGUUGCAGC |
| siRNA 850 | 850 | CAACUGAGACGAAUUUGCC | 7204 | GGCAAAUUCGUCUCAGUUG |
| siRNA 851 | 851 | AACUGAGACGAAUUUGCCG | 7205 | CGGCAAAUUCGUCUCAGUU |
| siRNA 852 | 852 | ACUGAGACGAAUUUGCCGC | 7206 | GCGGCAAAUUCGUCUCAGU |
| siRNA 853 | 853 | CUGAGACGAAUUUGCCGCU | 7207 | AGCGGCAAAUUCGUCUCAG |
| siRNA 854 | 854 | UGAGACGAAUUUGCCGCUU | 7208 | AAGCGGCAAAUUCGUCUCA |
| siRNA 855 | 855 | GAGACGAAUUUGCCGCUUC | 7209 | GAAGCGGCAAAUUCGUCUC |
| siRNA 857 | 857 | GACGAAUUUGCCGCUUCUG | 7211 | CAGAAGCGGCAAAUUCGUC |
| siRNA 859 | 859 | CGAAUUUGCCGCUUCUGUU | 7213 | AACAGAAGCGGCAAAUUCG |
| siRNA 867 | 867 | CCGCUUCUGUUUCUACCAG | 7221 | CUGGUAGAAACAGAAGCGG |
| siRNA 874 | 874 | UGUUUCUACCAGUUCAUAG | 7228 | CUAUGAACUGGUAGAAACA |
| siRNA 878 | 878 | UCUACCAGUUCAUAGAUCC | 7232 | GGAUCUAUGAACUGGUAGA |
| siRNA 879 | 879 | CUACCAGUUCAUAGAUCCC | 7233 | GGGAUCUAUGAACUGGUAG |
| siRNA 880 | 880 | UACCAGUUCAUAGAUCCCA | 7234 | UGGGAUCUAUGAACUGGUA |
| siRNA 887 | 887 | UCAUAGAUCCCAUAUUGAC | 7241 | GUCAAUAUGGGAUCUAUGA |
| siRNA 889 | 889 | AUAGAUCCCAUAUUGACUA | 7243 | UAGUCAAUAUGGGAUCUAU |
| siRNA 891 | 891 | AGAUCCCAUAUUGACUAUC | 7245 | GAUAGUCAAUAUGGGAUCU |
| siRNA 892 | 892 | GAUCCCAUAUUGACUAUCU | 7246 | AGAUAGUCAAUAUGGGAUC |
| siRNA 893 | 893 | AUCCCAUAUUGACUAUCUG | 7247 | CAGAUAGUCAAUAUGGGAU |
| siRNA 894 | 894 | UCCCAUAUUGACUAUCUGC | 7248 | GCAGAUAGUCAAUAUGGGA |
| siRNA 895 | 895 | CCCAUAUUGACUAUCUGCU | 7249 | AGCAGAUAGUCAAUAUGGG |
| siRNA 896 | 896 | CCAUAUUGACUAUCUGCUG | 7250 | CAGCAGAUAGUCAAUAUGG |
| siRNA 933 | 933 | UGCCAUAACAUCAAAGCAC | 7287 | GUGCUUUGAUGUUAUGGCA |
| siRNA 939 | 939 | AACAUCAAAGCACCAUACA | 7293 | UGUAUGGUGCUUUGAUGUU |
| siRNA 944 | 944 | CAAAGCACCAUACAUUGCU | 7298 | AGCAAUGUAUGGUGCUUUG |
| siRNA 945 | 945 | AAAGCACCAUACAUUGCUU | 7299 | AAGCAAUGUAUGGUGCUUU |
| siRNA 956 | 956 | CAUUGCUUCAGGCAAUAAU | 7310 | AUUAUUGCCUGAAGCAAUG |
| siRNA 959 | 959 | UGCUUCAGGCAAUAAUCUC | 7313 | GAGAUUAUUGCCUGAAGCA |
| siRNA 960 | 960 | GCUUCAGGCAAUAAUCUCA | 7314 | UGAGAUUAUUGCCUGAAGC |
| siRNA 963 | 963 | UCAGGCAAUAAUCUCAACA | 7317 | UGUUGAGAUUAUUGCCUGA |
| siRNA 964 | 964 | CAGGCAAUAAUCUCAACAU | 7318 | AUGUUGAGAUUAUUGCCUG |
| siRNA 966 | 966 | GGCAAUAAUCUCAACAUCC | 7320 | GGAUGUUGAGAUUAUUGCC |
| siRNA 974 | 974 | UCUCAACAUCCCAAUCUUC | 7328 | GAAGAUUGGGAUGUUGAGA |
| siRNA 984 | 984 | CCAAUCUUCAGUACCUUGA | 7338 | UCAAGGUACUGAAGAUUGG |
| siRNA 991 | 991 | UCAGUACCUUGAUCCAUAA | 7345 | UUAUGGAUCAAGGUACUGA |
| siRNA 993 | 993 | AGUACCUUGAUCCAUAAGC | 7347 | GCUUAUGGAUCAAGGUACU |
| siRNA 994 | 994 | GUACCUUGAUCCAUAAGCU | 7348 | AGCUUAUGGAUCAAGGUAC |
| siRNA 997 | 997 | CCUUGAUCCAUAAGCUUGG | 7351 | CCAAGCUUAUGGAUCAAGG |
| siRNA 998 | 998 | CUUGAUCCAUAAGCUUGGG | 7352 | CCCAAGCUUAUGGAUCAAG |
| siRNA 1000 | 1000 | UGAUCCAUAAGCUUGGGGG | 7354 | CCCCCAAGCUUAUGGAUCA |
| siRNA 1001 | 1001 | GAUCCAUAAGCUUGGGGGC | 7355 | GCCCCCAAGCUUAUGGAUC |
| siRNA 1005 | 1005 | CAUAAGCUUGGGGGCUUCU | 7359 | AGAAGCCCCCAAGCUUAUG |
| siRNA 1006 | 1006 | AUAAGCUUGGGGGCUUCUU | 7360 | AAGAAGCCCCCAAGCUUAU |
| siRNA 1018 | 1018 | GCUUCUUCAUACGACGAAG | 7372 | CUUCGUCGUAUGAAGAAGC |
| siRNA 1019 | 1019 | CUUCUUCAUACGACGAAGG | 7373 | CCUUCGUCGUAUGAAGAAG |
| siRNA 1021 | 1021 | UCUUCAUACGACGAAGGCU | 7375 | AGCCUUCGUCGUAUGAAGA |
| siRNA 1022 | 1022 | CUUCAUACGACGAAGGCUC | 7376 | GAGCCUUCGUCGUAUGAAG |
| siRNA 1023 | 1023 | UUCAUACGACGAAGGCUCG | 7377 | CGAGCCUUCGUCGUAUGAA |
| siRNA 1024 | 1024 | UCAUACGACGAAGGCUCGA | 7378 | UCGAGCCUUCGUCGUAUGA |
| siRNA 1025 | 1025 | CAUACGACGAAGGCUCGAU | 7379 | AUCGAGCCUUCGUCGUAUG |
| siRNA 1026 | 1026 | AUACGACGAAGGCUCGAUG | 7380 | CAUCGAGCCUUCGUCGUAU |
| siRNA 1029 | 1029 | CGACGAAGGCUCGAUGAAA | 7383 | UUUCAUCGAGCCUUCGUCG |
| siRNA 1030 | 1030 | GACGAAGGCUCGAUGAAAC | 7384 | GUUUCAUCGAGCCUUCGUC |
| siRNA 1031 | 1031 | ACGAAGGCUCGAUGAAACA | 7385 | UGUUUCAUCGAGCCUUCGU |
| siRNA 1032 | 1032 | CGAAGGCUCGAUGAAACAC | 7386 | GUGUUUCAUCGAGCCUUCG |
| siRNA 1033 | 1033 | GAAGGCUCGAUGAAACACC | 7387 | GGUGUUUCAUCGAGCCUUC |
| siRNA 1034 | 1034 | AAGGCUCGAUGAAACACCA | 7388 | UGGUGUUUCAUCGAGCCUU |
| siRNA 1035 | 1035 | AGGCUCGAUGAAACACCAG | 7389 | CUGGUGUUUCAUCGAGCCU |
| siRNA 1044 | 1044 | GAAACACCAGAUGGACGGA | 7398 | UCCGUCCAUCUGGUGUUUC |
| siRNA 1046 | 1046 | AACACCAGAUGGACGGAAA | 7400 | UUUCCGUCCAUCUGGUGUU |
| siRNA 1048 | 1048 | CACCAGAUGGACGGAAAGA | 7402 | UCUUUCCGUCCAUCUGGUG |
| siRNA 1052 | 1052 | AGAUGGACGGAAAGAUGUU | 7406 | AACAUCUUUCCGUCCAUCU |
| siRNA 1054 | 1054 | AUGGACGGAAAGAUGUUCU | 7408 | AGAACAUCUUUCCGUCCAU |
| siRNA 1058 | 1058 | ACGGAAAGAUGUUCUCUAU | 7412 | AUAGAGAACAUCUUUCCGU |
| siRNA 1065 | 1065 | GAUGUUCUCUAUAGAGCUU | 7419 | AAGCUCUAUAGAGAACAUC |
| siRNA 1071 | 1071 | CUCUAUAGAGCUUUGCUCC | 7425 | GGAGCAAAGCUCUAUAGAG |
| siRNA 1072 | 1072 | UCUAUAGAGCUUUGCUCCA | 7426 | UGGAGCAAAGCUCUAUAGA |
| siRNA 1079 | 1079 | AGCUUUGCUCCAUGGGCAU | 7433 | AUGCCCAUGGAGCAAAGCU |
| siRNA 1084 | 1084 | UGCUCCAUGGGCAUAUAGU | 7438 | ACUAUAUGCCCAUGGAGCA |
| siRNA 1086 | 1086 | CUCCAUGGGCAUAUAGUUG | 7440 | CAACUAUAUGCCCAUGGAG |
| siRNA 1087 | 1087 | UCCAUGGGCAUAUAGUUGA | 7441 | UCAACUAUAUGCCCAUGGA |
| siRNA 1091 | 1091 | UGGGCAUAUAGUUGAAUUA | 7445 | UAAUUCAACUAUAUGCCCA |
| siRNA 1092 | 1092 | GGGCAUAUAGUUGAAUUAC | 7446 | GUAAUUCAACUAUAUGCCC |
| siRNA 1093 | 1093 | GGCAUAUAGUUGAAUUACU | 7447 | AGUAAUUCAACUAUAUGCC |
| siRNA 1096 | 1096 | AUAUAGUUGAAUUACUUCG | 7450 | CGAAGUAAUUCAACUAUAU |
| siRNA 1097 | 1097 | UAUAGUUGAAUUACUUCGA | 7451 | UCGAAGUAAUUCAACUAUA |
| siRNA 1098 | 1098 | AUAGUUGAAUUACUUCGAC | 7452 | GUCGAAGUAAUUCAACUAU |
| siRNA 1099 | 1099 | UAGUUGAAUUACUUCGACA | 7453 | UGUCGAAGUAAUUCAACUA |
| siRNA 1100 | 1100 | AGUUGAAUUACUUCGACAG | 7454 | CUGUCGAAGUAAUUCAACU |
| siRNA 1102 | 1102 | UUGAAUUACUUCGACAGCA | 7456 | UGCUGUCGAAGUAAUUCAA |
| siRNA 1105 | 1105 | AAUUACUUCGACAGCAGCA | 7459 | UGCUGCUGUCGAAGUAAUU |
| siRNA 1106 | 1106 | AUUACUUCGACAGCAGCAA | 7460 | UUGCUGCUGUCGAAGUAAU |
| siRNA 1111 | 1111 | UUCGACAGCAGCAAUUCUU | 7465 | AAGAAUUGCUGCUGUCGAA |
| siRNA 1113 | 1113 | CGACAGCAGCAAUUCUUGG | 7467 | CCAAGAAUUGCUGCUGUCG |
| siRNA 1121 | 1121 | GCAAUUCUUGGAGAUCUUC | 7475 | GAAGAUCUCCAAGAAUUGC |
| siRNA 1122 | 1122 | CAAUUCUUGGAGAUCUUCC | 7476 | GGAAGAUCUCCAAGAAUUG |
| siRNA 1134 | 1134 | AUCUUCCUGGAAGGCACAC | 7488 | GUGUGCCUUCCAGGAAGAU |
| siRNA 1135 | 1135 | UCUUCCUGGAAGGCACACG | 7489 | CGUGUGCCUUCCAGGAAGA |
| siRNA 1136 | 1136 | CUUCCUGGAAGGCACACGU | 7490 | ACGUGUGCCUUCCAGGAAG |
| siRNA 1140 | 1140 | CUGGAAGGCACACGUUCUA | 7494 | UAGAACGUGUGCCUUCCAG |
| siRNA 1141 | 1141 | UGGAAGGCACACGUUCUAG | 7495 | CUAGAACGUGUGCCUUCCA |
| siRNA 1142 | 1142 | GGAAGGCACACGUUCUAGG | 7496 | CCUAGAACGUGUGCCUUCC |
| siRNA 1145 | 1145 | AGGCACACGUUCUAGGAGU | 7499 | ACUCCUAGAACGUGUGCCU |
| siRNA 1151 | 1151 | ACGUUCUAGGAGUGGAAAA | 7505 | UUUUCCACUCCUAGAACGU |
| siRNA 1152 | 1152 | CGUUCUAGGAGUGGAAAAA | 7506 | UUUUUCCACUCCUAGAACG |
| siRNA 1166 | 1166 | AAAAACCUCUUGUGCUCGG | 7520 | CCGAGCACAAGAGGUUUUU |
| siRNA 1167 | 1167 | AAAACCUCUUGUGCUCGGG | 7521 | CCCGAGCACAAGAGGUUUU |
| siRNA 1169 | 1169 | AACCUCUUGUGCUCGGGCA | 7523 | UGCCCGAGCACAAGAGGUU |
| siRNA 1177 | 1177 | GUGCUCGGGCAGGACUUUU | 7531 | AAAAGUCCUGCCCGAGCAC |
| siRNA 1180 | 1180 | CUCGGGCAGGACUUUUGUC | 7534 | GACAAAAGUCCUGCCCGAG |
| siRNA 1181 | 1181 | UCGGGCAGGACUUUUGUCA | 7535 | UGACAAAAGUCCUGCCCGA |
| siRNA 1191 | 1191 | CUUUUGUCAGUUGUGGUAG | 7545 | CUACCACAACUGACAAAAG |
| siRNA 1194 | 1194 | UUGUCAGUUGUGGUAGAUA | 7548 | UAUCUACCACAACUGACAA |
| siRNA 1196 | 1196 | GUCAGUUGUGGUAGAUACU | 7550 | AGUAUCUACCACAACUGAC |
| siRNA 1197 | 1197 | UCAGUUGUGGUAGAUACUC | 7551 | GAGUAUCUACCACAACUGA |
| siRNA 1199 | 1199 | AGUUGUGGUAGAUACUCUG | 7553 | CAGAGUAUCUACCACAACU |
| siRNA 1202 | 1202 | UGUGGUAGAUACUCUGUCU | 7556 | AGACAGAGUAUCUACCACA |
| siRNA 1207 | 1207 | UAGAUACUCUGUCUACCAA | 7561 | UUGGUAGACAGAGUAUCUA |
| siRNA 1208 | 1208 | AGAUACUCUGUCUACCAAU | 7562 | AUUGGUAGACAGAGUAUCU |
| siRNA 1209 | 1209 | GAUACUCUGUCUACCAAUG | 7563 | CAUUGGUAGACAGAGUAUC |
| siRNA 1210 | 1210 | AUACUCUGUCUACCAAUGU | 7564 | ACAUUGGUAGACAGAGUAU |
| siRNA 1211 | 1211 | UACUCUGUCUACCAAUGUC | 7565 | GACAUUGGUAGACAGAGUA |
| siRNA 1212 | 1212 | ACUCUGUCUACCAAUGUCA | 7566 | UGACAUUGGUAGACAGAGU |
| siRNA 1213 | 1213 | CUCUGUCUACCAAUGUCAU | 7567 | AUGACAUUGGUAGACAGAG |
| siRNA 1217 | 1217 | GUCUACCAAUGUCAUCCCA | 7571 | UGGGAUGACAUUGGUAGAC |
| siRNA 1230 | 1230 | AUCCCAGACAUCUUGAUAA | 7584 | UUAUCAAGAUGUCUGGGAU |
| siRNA 1231 | 1231 | UCCCAGACAUCUUGAUAAU | 7585 | AUUAUCAAGAUGUCUGGGA |
| siRNA 1233 | 1233 | CCAGACAUCUUGAUAAUAC | 7587 | GUAUUAUCAAGAUGUCUGG |
| siRNA 1236 | 1236 | GACAUCUUGAUAAUACCUG | 7590 | CAGGUAUUAUCAAGAUGUC |
| siRNA 1241 | 1241 | CUUGAUAAUACCUGUUGGA | 7595 | UCCAACAGGUAUUAUCAAG |
| siRNA 1243 | 1243 | UGAUAAUACCUGUUGGAAU | 7597 | AUUCCAACAGGUAUUAUCA |
| siRNA 1245 | 1245 | AUAAUACCUGUUGGAAUCU | 7599 | AGAUUCCAACAGGUAUUAU |
| siRNA 1246 | 1246 | UAAUACCUGUUGGAAUCUC | 7600 | GAGAUUCCAACAGGUAUUA |
| siRNA 1249 | 1249 | UACCUGUUGGAAUCUCCUA | 7603 | UAGGAGAUUCCAACAGGUA |
| siRNA 1252 | 1252 | CUGUUGGAAUCUCCUAUGA | 7606 | UCAUAGGAGAUUCCAACAG |
| siRNA 1255 | 1255 | UUGGAAUCUCCUAUGAUCG | 7609 | CGAUCAUAGGAGAUUCCAA |
| siRNA 1256 | 1256 | UGGAAUCUCCUAUGAUCGC | 7610 | GCGAUCAUAGGAGAUUCCA |
| siRNA 1257 | 1257 | GGAAUCUCCUAUGAUCGCA | 7611 | UGCGAUCAUAGGAGAUUCC |
| siRNA 1259 | 1259 | AAUCUCCUAUGAUCGCAUU | 7613 | AAUGCGAUCAUAGGAGAUU |
| siRNA 1260 | 1260 | AUCUCCUAUGAUCGCAUUA | 7614 | UAAUGCGAUCAUAGGAGAU |
| siRNA 1263 | 1263 | UCCUAUGAUCGCAUUAUCG | 7617 | CGAUAAUGCGAUCAUAGGA |
| siRNA 1264 | 1264 | CCUAUGAUCGCAUUAUCGA | 7618 | UCGAUAAUGCGAUCAUAGG |
| siRNA 1265 | 1265 | CUAUGAUCGCAUUAUCGAA | 7619 | UUCGAUAAUGCGAUCAUAG |
| siRNA 1266 | 1266 | UAUGAUCGCAUUAUCGAAG | 7620 | CUUCGAUAAUGCGAUCAUA |
| siRNA 1267 | 1267 | AUGAUCGCAUUAUCGAAGG | 7621 | CCUUCGAUAAUGCGAUCAU |
| siRNA 1268 | 1268 | UGAUCGCAUUAUCGAAGGU | 7622 | ACCUUCGAUAAUGCGAUCA |
| siRNA 1269 | 1269 | GAUCGCAUUAUCGAAGGUC | 7623 | GACCUUCGAUAAUGCGAUC |
| siRNA 1270 | 1270 | AUCGCAUUAUCGAAGGUCA | 7624 | UGACCUUCGAUAAUGCGAU |
| siRNA 1272 | 1272 | CGCAUUAUCGAAGGUCACU | 7626 | AGUGACCUUCGAUAAUGCG |
| siRNA 1279 | 1279 | UCGAAGGUCACUACAAUGG | 7633 | CCAUUGUAGUGACCUUCGA |
| siRNA 1281 | 1281 | GAAGGUCACUACAAUGGUG | 7635 | CACCAUUGUAGUGACCUUC |
| siRNA 1283 | 1283 | AGGUCACUACAAUGGUGAA | 7637 | UUCACCAUUGUAGUGACCU |
| siRNA 1285 | 1285 | GUCACUACAAUGGUGAACA | 7639 | UGUUCACCAUUGUAGUGAC |
| siRNA 1286 | 1286 | UCACUACAAUGGUGAACAA | 7640 | UUGUUCACCAUUGUAGUGA |
| siRNA 1288 | 1288 | ACUACAAUGGUGAACAACU | 7642 | AGUUGUUCACCAUUGUAGU |
| siRNA 1289 | 1289 | CUACAAUGGUGAACAACUG | 7643 | CAGUUGUUCACCAUUGUAG |
| siRNA 1297 | 1297 | GUGAACAACUGGGCAAACC | 7651 | GGUUUGCCCAGUUGUUCAC |
| siRNA 1299 | 1299 | GAACAACUGGGCAAACCUA | 7653 | UAGGUUUGCCCAGUUGUUC |
| siRNA 1300 | 1300 | AACAACUGGGCAAACCUAA | 7654 | UUAGGUUUGCCCAGUUGUU |
| siRNA 1301 | 1301 | ACAACUGGGCAAACCUAAG | 7655 | CUUAGGUUUGCCCAGUUGU |
| siRNA 1305 | 1305 | CUGGGCAAACCUAAGAAGA | 7659 | UCUUCUUAGGUUUGCCCAG |
| siRNA 1308 | 1308 | GGCAAACCUAAGAAGAAUG | 7662 | CAUUCUUCUUAGGUUUGCC |
| siRNA 1329 | 1329 | AGCCUGUGGAGUGUAGCAA | 7683 | UUGCUACACUCCACAGGCU |
| siRNA 1330 | 1330 | GCCUGUGGAGUGUAGCAAG | 7684 | CUUGCUACACUCCACAGGC |
| siRNA 1333 | 1333 | UGUGGAGUGUAGCAAGAGG | 7687 | CCUCUUGCUACACUCCACA |
| siRNA 1339 | 1339 | GUGUAGCAAGAGGUGUUAU | 7693 | AUAACACCUCUUGCUACAC |
| siRNA 1340 | 1340 | UGUAGCAAGAGGUGUUAUU | 7694 | AAUAACACCUCUUGCUACA |
| siRNA 1341 | 1341 | GUAGCAAGAGGUGUUAUUA | 7695 | UAAUAACACCUCUUGCUAC |
| siRNA 1343 | 1343 | AGCAAGAGGUGUUAUUAGA | 7697 | UCUAAUAACACCUCUUGCU |
| siRNA 1346 | 1346 | AAGAGGUGUUAUUAGAAUG | 7700 | CAUUCUAAUAACACCUCUU |
| siRNA 1350 | 1350 | GGUGUUAUUAGAAUGUUAC | 7704 | GUAACAUUCUAAUAACACC |
| siRNA 1352 | 1352 | UGUUAUUAGAAUGUUACGA | 7706 | UCGUAACAUUCUAAUAACA |
| siRNA 1354 | 1354 | UUAUUAGAAUGUUACGAAA | 7708 | UUUCGUAACAUUCUAAUAA |
| siRNA 1355 | 1355 | UAUUAGAAUGUUACGAAAA | 7709 | UUUUCGUAACAUUCUAAUA |
| siRNA 1358 | 1358 | UAGAAUGUUACGAAAAAAC | 7712 | GUUUUUUCGUAACAUUCUA |
| siRNA 1359 | 1359 | AGAAUGUUACGAAAAAACU | 7713 | AGUUUUUUCGUAACAUUCU |
| siRNA 1361 | 1361 | AAUGUUACGAAAAAACUAU | 7715 | AUAGUUUUUUCGUAACAUU |
| siRNA 1363 | 1363 | UGUUACGAAAAAACUAUGG | 7717 | CCAUAGUUUUUUCGUAACA |
| siRNA 1367 | 1367 | ACGAAAAAACUAUGGUUGU | 7721 | ACAACCAUAGUUUUUUCGU |
| siRNA 1373 | 1373 | AAACUAUGGUUGUGUCCGA | 7727 | UCGGACACAACCAUAGUUU |
| siRNA 1374 | 1374 | AACUAUGGUUGUGUCCGAG | 7728 | CUCGGACACAACCAUAGUU |
| siRNA 1426 | 1426 | AUUUAGAAAGCCAAAGUCA | 7780 | UGACUUUGGCUUUCUAAAU |
| siRNA 1432 | 1432 | AAAGCCAAAGUCAGAAACC | 7786 | GGUUUCUGACUUUGGCUUU |
| siRNA 1434 | 1434 | AGCCAAAGUCAGAAACCGG | 7788 | CCGGUUUCUGACUUUGGCU |
| siRNA 1436 | 1436 | CCAAAGUCAGAAACCGGUG | 7790 | CACCGGUUUCUGACUUUGG |
| siRNA 1461 | 1461 | CUACUUUCCCUGGAGCAAG | 7815 | CUUGCUCCAGGGAAAGUAG |
| siRNA 1464 | 1464 | CUUUCCCUGGAGCAAGCGU | 7818 | ACGCUUGCUCCAGGGAAAG |
| siRNA 1466 | 1466 | UUCCCUGGAGCAAGCGUUG | 7820 | CAACGCUUGCUCCAGGGAA |
| siRNA 1467 | 1467 | UCCCUGGAGCAAGCGUUGU | 7821 | ACAACGCUUGCUCCAGGGA |
| siRNA 1468 | 1468 | CCCUGGAGCAAGCGUUGUU | 7822 | AACAACGCUUGCUCCAGGG |
| siRNA 1469 | 1469 | CCUGGAGCAAGCGUUGUUA | 7823 | UAACAACGCUUGCUCCAGG |
| siRNA 1470 | 1470 | CUGGAGCAAGCGUUGUUAC | 7824 | GUAACAACGCUUGCUCCAG |
| siRNA 1472 | 1472 | GGAGCAAGCGUUGUUACCA | 7826 | UGGUAACAACGCUUGCUCC |
| siRNA 1473 | 1473 | GAGCAAGCGUUGUUACCAG | 7827 | CUGGUAACAACGCUUGCUC |
| siRNA 1474 | 1474 | AGCAAGCGUUGUUACCAGC | 7828 | GCUGGUAACAACGCUUGCU |
| siRNA 1476 | 1476 | CAAGCGUUGUUACCAGCUA | 7830 | UAGCUGGUAACAACGCUUG |
| siRNA 1477 | 1477 | AAGCGUUGUUACCAGCUAU | 7831 | AUAGCUGGUAACAACGCUU |
| siRNA 1478 | 1478 | AGCGUUGUUACCAGCUAUA | 7832 | UAUAGCUGGUAACAACGCU |
| siRNA 1479 | 1479 | GCGUUGUUACCAGCUAUAC | 7833 | GUAUAGCUGGUAACAACGC |
| siRNA 1480 | 1480 | CGUUGUUACCAGCUAUACU | 7834 | AGUAUAGCUGGUAACAACG |
| siRNA 1481 | 1481 | GUUGUUACCAGCUAUACUU | 7835 | AAGUAUAGCUGGUAACAAC |
| siRNA 1482 | 1482 | UUGUUACCAGCUAUACUUC | 7836 | GAAGUAUAGCUGGUAACAA |
| siRNA 1483 | 1483 | UGUUACCAGCUAUACUUCC | 7837 | GGAAGUAUAGCUGGUAACA |
| siRNA 1484 | 1484 | GUUACCAGCUAUACUUCCU | 7838 | AGGAAGUAUAGCUGGUAAC |
| siRNA 1488 | 1488 | CCAGCUAUACUUCCUUCAA | 7842 | UUGAAGGAAGUAUAGCUGG |
| siRNA 1525 | 1525 | AUGAAGGUAGAGACACGUC | 7879 | GACGUGUCUCUACCUUCAU |
| siRNA 1530 | 1530 | GGUAGAGACACGUCCAUUA | 7884 | UAAUGGACGUGUCUCUACC |
| siRNA 1531 | 1531 | GUAGAGACACGUCCAUUAA | 7885 | UUAAUGGACGUGUCUCUAC |
| siRNA 1533 | 1533 | AGAGACACGUCCAUUAAUG | 7887 | CAUUAAUGGACGUGUCUCU |
| siRNA 1534 | 1534 | GAGACACGUCCAUUAAUGA | 7888 | UCAUUAAUGGACGUGUCUC |
| siRNA 1537 | 1537 | ACACGUCCAUUAAUGAGUC | 7891 | GACUCAUUAAUGGACGUGU |
| siRNA 1538 | 1538 | CACGUCCAUUAAUGAGUCC | 7892 | GGACUCAUUAAUGGACGUG |
| siRNA 1539 | 1539 | ACGUCCAUUAAUGAGUCCA | 7893 | UGGACUCAUUAAUGGACGU |
| siRNA 1540 | 1540 | CGUCCAUUAAUGAGUCCAG | 7894 | CUGGACUCAUUAAUGGACG |
| siRNA 1542 | 1542 | UCCAUUAAUGAGUCCAGAA | 7896 | UUCUGGACUCAUUAAUGGA |
| siRNA 1543 | 1543 | CCAUUAAUGAGUCCAGAAA | 7897 | UUUCUGGACUCAUUAAUGG |
| siRNA 1546 | 1546 | UUAAUGAGUCCAGAAAUGC | 7900 | GCAUUUCUGGACUCAUUAA |
| siRNA 1565 | 1565 | AACAGAUGAAUCCCUACGA | 7919 | UCGUAGGGAUUCAUCUGUU |
| siRNA 1566 | 1566 | ACAGAUGAAUCCCUACGAA | 7920 | UUCGUAGGGAUUCAUCUGU |
| siRNA 1569 | 1569 | GAUGAAUCCCUACGAAGGA | 7923 | UCCUUCGUAGGGAUUCAUC |
| siRNA 1570 | 1570 | AUGAAUCCCUACGAAGGAG | 7924 | CUCCUUCGUAGGGAUUCAU |
| siRNA 1571 | 1571 | UGAAUCCCUACGAAGGAGG | 7925 | CCUCCUUCGUAGGGAUUCA |
| siRNA 1572 | 1572 | GAAUCCCUACGAAGGAGGU | 7926 | ACCUCCUUCGUAGGGAUUC |
| siRNA 1574 | 1574 | AUCCCUACGAAGGAGGUUG | 7928 | CAACCUCCUUCGUAGGGAU |
| siRNA 1577 | 1577 | CCUACGAAGGAGGUUGAUU | 7931 | AAUCAACCUCCUUCGUAGG |
| siRNA 1578 | 1578 | CUACGAAGGAGGUUGAUUG | 7932 | CAAUCAACCUCCUUCGUAG |
| siRNA 1580 | 1580 | ACGAAGGAGGUUGAUUGCA | 7934 | UGCAAUCAACCUCCUUCGU |
| siRNA 1582 | 1582 | GAAGGAGGUUGAUUGCAAA | 7936 | UUUGCAAUCAACCUCCUUC |
| siRNA 1584 | 1584 | AGGAGGUUGAUUGCAAAUC | 7938 | GAUUUGCAAUCAACCUCCU |
| siRNA 1586 | 1586 | GAGGUUGAUUGCAAAUCUG | 7940 | CAGAUUUGCAAUCAACCUC |
| siRNA 1600 | 1600 | AUCUGGCUGAGCAUAUUCU | 7954 | AGAAUAUGCUCAGCCAGAU |
| siRNA 1608 | 1608 | GAGCAUAUUCUAUUCACUG | 7962 | CAGUGAAUAGAAUAUGCUC |
| siRNA 1613 | 1613 | UAUUCUAUUCACUGCUAGC | 7967 | GCUAGCAGUGAAUAGAAUA |
| siRNA 1614 | 1614 | AUUCUAUUCACUGCUAGCA | 7968 | UGCUAGCAGUGAAUAGAAU |
| siRNA 1615 | 1615 | UUCUAUUCACUGCUAGCAA | 7969 | UUGCUAGCAGUGAAUAGAA |
| siRNA 1617 | 1617 | CUAUUCACUGCUAGCAAGU | 7971 | ACUUGCUAGCAGUGAAUAG |
| siRNA 1618 | 1618 | UAUUCACUGCUAGCAAGUC | 7972 | GACUUGCUAGCAGUGAAUA |
| siRNA 1619 | 1619 | AUUCACUGCUAGCAAGUCC | 7973 | GGACUUGCUAGCAGUGAAU |
| siRNA 1622 | 1622 | CACUGCUAGCAAGUCCUGU | 7976 | ACAGGACUUGCUAGCAGUG |
| siRNA 1625 | 1625 | UGCUAGCAAGUCCUGUGCC | 7979 | GGCACAGGACUUGCUAGCA |
| siRNA 1634 | 1634 | GUCCUGUGCCAUUAUGUCC | 7988 | GGACAUAAUGGCACAGGAC |
| siRNA 1635 | 1635 | UCCUGUGCCAUUAUGUCCA | 7989 | UGGACAUAAUGGCACAGGA |
| siRNA 1639 | 1639 | GUGCCAUUAUGUCCACACA | 7993 | UGUGUGGACAUAAUGGCAC |
| siRNA 1645 | 1645 | UUAUGUCCACACACAUUGU | 7999 | ACAAUGUGUGUGGACAUAA |
| siRNA 1647 | 1647 | AUGUCCACACACAUUGUGG | 8001 | CCACAAUGUGUGUGGACAU |
| siRNA 1650 | 1650 | UCCACACACAUUGUGGCUU | 8004 | AAGCCACAAUGUGUGUGGA |
| siRNA 1651 | 1651 | CCACACACAUUGUGGCUUG | 8005 | CAAGCCACAAUGUGUGUGG |
| siRNA 1655 | 1655 | ACACAUUGUGGCUUGCCUG | 8009 | CAGGCAAGCCACAAUGUGU |
| siRNA 1656 | 1656 | CACAUUGUGGCUUGCCUGC | 8010 | GCAGGCAAGCCACAAUGUG |
| siRNA 1686 | 1686 | CACAGGCAGGGAAUUGAUC | 8040 | GAUCAAUUCCCUGCCUGUG |
| siRNA 1688 | 1688 | CAGGCAGGGAAUUGAUCUC | 8042 | GAGAUCAAUUCCCUGCCUG |
| siRNA 1689 | 1689 | AGGCAGGGAAUUGAUCUCU | 8043 | AGAGAUCAAUUCCCUGCCU |
| siRNA 1693 | 1693 | AGGGAAUUGAUCUCUCCAC | 8047 | GUGGAGAGAUCAAUUCCCU |
| siRNA 1697 | 1697 | AAUUGAUCUCUCCACAUUG | 8051 | CAAUGUGGAGAGAUCAAUU |
| siRNA 1702 | 1702 | AUCUCUCCACAUUGGUCGA | 8056 | UCGACCAAUGUGGAGAGAU |
| siRNA 1704 | 1704 | CUCUCCACAUUGGUCGAAG | 8058 | CUUCGACCAAUGUGGAGAG |
| siRNA 1705 | 1705 | UCUCCACAUUGGUCGAAGA | 8059 | UCUUCGACCAAUGUGGAGA |
| siRNA 1706 | 1706 | CUCCACAUUGGUCGAAGAC | 8060 | GUCUUCGACCAAUGUGGAG |
| siRNA 1707 | 1707 | UCCACAUUGGUCGAAGACU | 8061 | AGUCUUCGACCAAUGUGGA |
| siRNA 1708 | 1708 | CCACAUUGGUCGAAGACUU | 8062 | AAGUCUUCGACCAAUGUGG |
| siRNA 1709 | 1709 | CACAUUGGUCGAAGACUUC | 8063 | GAAGUCUUCGACCAAUGUG |
| siRNA 1710 | 1710 | ACAUUGGUCGAAGACUUCU | 8064 | AGAAGUCUUCGACCAAUGU |
| siRNA 1733 | 1733 | GAUGAAAGAGGAAGUCCUG | 8087 | CAGGACUUCCUCUUUCAUC |
| siRNA 1736 | 1736 | GAAAGAGGAAGUCCUGGCU | 8090 | AGCCAGGACUUCCUCUUUC |
| siRNA 1742 | 1742 | GGAAGUCCUGGCUCGUGAU | 8096 | AUCACGAGCCAGGACUUCC |
| siRNA 1744 | 1744 | AAGUCCUGGCUCGUGAUUU | 8098 | AAAUCACGAGCCAGGACUU |
| siRNA 1746 | 1746 | GUCCUGGCUCGUGAUUUUG | 8100 | CAAAAUCACGAGCCAGGAC |
| siRNA 1753 | 1753 | CUCGUGAUUUUGACCUGGG | 8107 | CCCAGGUCAAAAUCACGAG |
| siRNA 1754 | 1754 | UCGUGAUUUUGACCUGGGG | 8108 | CCCCAGGUCAAAAUCACGA |
| siRNA 1757 | 1757 | UGAUUUUGACCUGGGGUUC | 8111 | GAACCCCAGGUCAAAAUCA |
| siRNA 1779 | 1779 | GGAAAUUCAGAAGAUGUAG | 8133 | CUACAUCUUCUGAAUUUCC |
| siRNA 1784 | 1784 | UUCAGAAGAUGUAGUAAUG | 8138 | CAUUACUACAUCUUCUGAA |
| siRNA 1786 | 1786 | CAGAAGAUGUAGUAAUGCA | 8140 | UGCAUUACUACAUCUUCUG |
| siRNA 1795 | 1795 | UAGUAAUGCAUGCCAUACA | 8149 | UGUAUGGCAUGCAUUACUA |
| siRNA 1818 | 1818 | CUGGGAAAUUGUGUCACAA | 8172 | UUGUGACACAAUUUCCCAG |
| siRNA 1824 | 1824 | AAUUGUGUCACAAUCACCC | 8178 | GGGUGAUUGUGACACAAUU |
| siRNA 1829 | 1829 | UGUCACAAUCACCCACACU | 8183 | AGUGUGGGUGAUUGUGACA |
| siRNA 1831 | 1831 | UCACAAUCACCCACACUAG | 8185 | CUAGUGUGGGUGAUUGUGA |
| siRNA 1832 | 1832 | CACAAUCACCCACACUAGC | 8186 | GCUAGUGUGGGUGAUUGUG |
| siRNA 1868 | 1868 | UAUCACCCCCAGCACAACU | 8222 | AGUUGUGCUGGGGGUGAUA |
| siRNA 1869 | 1869 | AUCACCCCCAGCACAACUG | 8223 | CAGUUGUGCUGGGGGUGAU |
| siRNA 1871 | 1871 | CACCCCCAGCACAACUGUC | 8225 | GACAGUUGUGCUGGGGGUG |
| siRNA 1872 | 1872 | ACCCCCAGCACAACUGUCC | 8226 | GGACAGUUGUGCUGGGGGU |
| siRNA 1873 | 1873 | CCCCCAGCACAACUGUCCC | 8227 | GGGACAGUUGUGCUGGGGG |
| siRNA 1877 | 1877 | CAGCACAACUGUCCCAUCA | 8231 | UGAUGGGACAGUUGUGCUG |
| siRNA 1880 | 1880 | CACAACUGUCCCAUCAGUC | 8234 | GACUGAUGGGACAGUUGUG |
| siRNA 1885 | 1885 | CUGUCCCAUCAGUCUUCGA | 8239 | UCGAAGACUGAUGGGACAG |
| siRNA 1886 | 1886 | UGUCCCAUCAGUCUUCGAA | 8240 | UUCGAAGACUGAUGGGACA |
| siRNA 1887 | 1887 | GUCCCAUCAGUCUUCGAAC | 8241 | GUUCGAAGACUGAUGGGAC |
| siRNA 1890 | 1890 | CCAUCAGUCUUCGAACUCA | 8244 | UGAGUUCGAAGACUGAUGG |
| siRNA 1891 | 1891 | CAUCAGUCUUCGAACUCAA | 8245 | UUGAGUUCGAAGACUGAUG |
| siRNA 1892 | 1892 | AUCAGUCUUCGAACUCAAC | 8246 | GUUGAGUUCGAAGACUGAU |
| siRNA 1894 | 1894 | CAGUCUUCGAACUCAACUU | 8248 | AAGUUGAGUUCGAAGACUG |
| siRNA 1896 | 1896 | GUCUUCGAACUCAACUUCU | 8250 | AGAAGUUGAGUUCGAAGAC |
| siRNA 1899 | 1899 | UUCGAACUCAACUUCUACA | 8253 | UGUAGAAGUUGAGUUCGAA |
| siRNA 1911 | 1911 | UUCUACAGCAAUGGGGUAC | 8265 | GUACCCCAUUGCUGUAGAA |
| siRNA 1915 | 1915 | ACAGCAAUGGGGUACUUCA | 8269 | UGAAGUACCCCAUUGCUGU |
| siRNA 1917 | 1917 | AGCAAUGGGGUACUUCAUG | 8271 | CAUGAAGUACCCCAUUGCU |
| siRNA 1918 | 1918 | GCAAUGGGGUACUUCAUGU | 8272 | ACAUGAAGUACCCCAUUGC |
| siRNA 1919 | 1919 | CAAUGGGGUACUUCAUGUC | 8273 | GACAUGAAGUACCCCAUUG |
| siRNA 1920 | 1920 | AAUGGGGUACUUCAUGUCU | 8274 | AGACAUGAAGUACCCCAUU |
| siRNA 1926 | 1926 | GUACUUCAUGUCUUUAUCA | 8280 | UGAUAAAGACAUGAAGUAC |
| siRNA 1930 | 1930 | UUCAUGUCUUUAUCAUGGA | 8284 | UCCAUGAUAAAGACAUGAA |
| siRNA 1934 | 1934 | UGUCUUUAUCAUGGAGGCC | 8288 | GGCCUCCAUGAUAAAGACA |
| siRNA 1938 | 1938 | UUUAUCAUGGAGGCCAUCA | 8292 | UGAUGGCCUCCAUGAUAAA |
| siRNA 1939 | 1939 | UUAUCAUGGAGGCCAUCAU | 8293 | AUGAUGGCCUCCAUGAUAA |
| siRNA 1940 | 1940 | UAUCAUGGAGGCCAUCAUA | 8294 | UAUGAUGGCCUCCAUGAUA |
| siRNA 1945 | 1945 | UGGAGGCCAUCAUAGCUUG | 8299 | CAAGCUAUGAUGGCCUCCA |
| siRNA 1946 | 1946 | GGAGGCCAUCAUAGCUUGC | 8300 | GCAAGCUAUGAUGGCCUCC |
| siRNA 1949 | 1949 | GGCCAUCAUAGCUUGCAGC | 8303 | GCUGCAAGCUAUGAUGGCC |
| siRNA 1950 | 1950 | GCCAUCAUAGCUUGCAGCC | 8304 | GGCUGCAAGCUAUGAUGGC |
| siRNA 1955 | 1955 | CAUAGCUUGCAGCCUUUAU | 8309 | AUAAAGGCUGCAAGCUAUG |
| siRNA 1957 | 1957 | UAGCUUGCAGCCUUUAUGC | 8311 | GCAUAAAGGCUGCAAGCUA |
| siRNA 1962 | 1962 | UGCAGCCUUUAUGCAGUUC | 8316 | GAACUGCAUAAAGGCUGCA |
| siRNA 1963 | 1963 | GCAGCCUUUAUGCAGUUCU | 8317 | AGAACUGCAUAAAGGCUGC |
| siRNA 1973 | 1973 | UGCAGUUCUGAACAAGAGG | 8327 | CCUCUUGUUCAGAACUGCA |
| siRNA 1975 | 1975 | CAGUUCUGAACAAGAGGGG | 8329 | CCCCUCUUGUUCAGAACUG |
| siRNA 1976 | 1976 | AGUUCUGAACAAGAGGGGA | 8330 | UCCCCUCUUGUUCAGAACU |
| siRNA 1977 | 1977 | GUUCUGAACAAGAGGGGAC | 8331 | GUCCCCUCUUGUUCAGAAC |
| siRNA 1978 | 1978 | UUCUGAACAAGAGGGGACU | 8332 | AGUCCCCUCUUGUUCAGAA |
| siRNA 1982 | 1982 | GAACAAGAGGGGACUGGGG | 8336 | CCCCAGUCCCCUCUUGUUC |
| siRNA 1992 | 1992 | GGACUGGGGGGUCCCACUA | 8346 | UAGUGGGACCCCCCAGUCC |
| siRNA 1993 | 1993 | GACUGGGGGGUCCCACUAG | 8347 | CUAGUGGGACCCCCCAGUC |
| siRNA 1996 | 1996 | UGGGGGGUCCCACUAGCAC | 8350 | GUGCUAGUGGGACCCCCCA |
| siRNA 1997 | 1997 | GGGGGGUCCCACUAGCACC | 8351 | GGUGCUAGUGGGACCCCCC |
| siRNA 1999 | 1999 | GGGGUCCCACUAGCACCCC | 8353 | GGGGUGCUAGUGGGACCCC |
| siRNA 2001 | 2001 | GGUCCCACUAGCACCCCAC | 8355 | GUGGGGUGCUAGUGGGACC |
| siRNA 2006 | 2006 | CACUAGCACCCCACCUAAC | 8360 | GUUAGGUGGGGUGCUAGUG |
| siRNA 2007 | 2007 | ACUAGCACCCCACCUAACC | 8361 | GGUUAGGUGGGGUGCUAGU |
| siRNA 2008 | 2008 | CUAGCACCCCACCUAACCU | 8362 | AGGUUAGGUGGGGUGCUAG |
| siRNA 2010 | 2010 | AGCACCCCACCUAACCUGA | 8364 | UCAGGUUAGGUGGGGUGCU |
| siRNA 2012 | 2012 | CACCCCACCUAACCUGAUC | 8366 | GAUCAGGUUAGGUGGGGUG |
| siRNA 2017 | 2017 | CACCUAACCUGAUCAGCCA | 8371 | UGGCUGAUCAGGUUAGGUG |
| siRNA 2039 | 2039 | GCAGCUGGUGCGGAAGGCG | 8393 | CGCCUUCCGCACCAGCUGC |
| siRNA 2055 | 2055 | GCGGCCAGCCUGUGCUACC | 8409 | GGUAGCACAGGCUGGCCGC |
| siRNA 2067 | 2067 | UGCUACCUUCUCUCCAAUG | 8421 | CAUUGGAGAGAAGGUAGCA |
| siRNA 2076 | 2076 | CUCUCCAAUGAAGGCACCA | 8430 | UGGUGCCUUCAUUGGAGAG |
| siRNA 2077 | 2077 | UCUCCAAUGAAGGCACCAU | 8431 | AUGGUGCCUUCAUUGGAGA |
| siRNA 2078 | 2078 | CUCCAAUGAAGGCACCAUC | 8432 | GAUGGUGCCUUCAUUGGAG |
| siRNA 2082 | 2082 | AAUGAAGGCACCAUCUCAC | 8436 | GUGAGAUGGUGCCUUCAUU |
| siRNA 2093 | 2093 | CAUCUCACUGCCUUGCCAG | 8447 | CUGGCAAGGCAGUGAGAUG |
| siRNA 2105 | 2105 | UUGCCAGACAUUUUACCAA | 8459 | UUGGUAAAAUGUCUGGCAA |
| siRNA 2110 | 2110 | AGACAUUUUACCAAGUCUG | 8464 | CAGACUUGGUAAAAUGUCU |
| siRNA 2112 | 2112 | ACAUUUUACCAAGUCUGCC | 8466 | GGCAGACUUGGUAAAAUGU |
| siRNA 2125 | 2125 | UCUGCCAUGAAACAGUAGG | 8479 | CCUACUGUUUCAUGGCAGA |
| siRNA 2126 | 2126 | CUGCCAUGAAACAGUAGGA | 8480 | UCCUACUGUUUCAUGGCAG |
| siRNA 2131 | 2131 | AUGAAACAGUAGGAAAGUU | 8485 | AACUUUCCUACUGUUUCAU |
| siRNA 2141 | 2141 | AGGAAAGUUUAUCCAGUAU | 8495 | AUACUGGAUAAACUUUCCU |
| siRNA 2142 | 2142 | GGAAAGUUUAUCCAGUAUG | 8496 | CAUACUGGAUAAACUUUCC |
| siRNA 2143 | 2143 | GAAAGUUUAUCCAGUAUGG | 8497 | CCAUACUGGAUAAACUUUC |
| siRNA 2146 | 2146 | AGUUUAUCCAGUAUGGCAU | 8500 | AUGCCAUACUGGAUAAACU |
| siRNA 2158 | 2158 | AUGGCAUUCUUACAGUGGC | 8512 | GCCACUGUAAGAAUGCCAU |
| siRNA 2160 | 2160 | GGCAUUCUUACAGUGGCAG | 8514 | CUGCCACUGUAAGAAUGCC |
| siRNA 2166 | 2166 | CUUACAGUGGCAGAGCACG | 8520 | CGUGCUCUGCCACUGUAAG |
| siRNA 2167 | 2167 | UUACAGUGGCAGAGCACGA | 8521 | UCGUGCUCUGCCACUGUAA |
| siRNA 2170 | 2170 | CAGUGGCAGAGCACGAUGA | 8524 | UCAUCGUGCUCUGCCACUG |
| siRNA 2171 | 2171 | AGUGGCAGAGCACGAUGAC | 8525 | GUCAUCGUGCUCUGCCACU |
| siRNA 2182 | 2182 | ACGAUGACCAGGAAGAUAU | 8536 | AUAUCUUCCUGGUCAUCGU |
| siRNA 2187 | 2187 | GACCAGGAAGAUAUCAGUC | 8541 | GACUGAUAUCUUCCUGGUC |
| siRNA 2190 | 2190 | CAGGAAGAUAUCAGUCCUA | 8544 | UAGGACUGAUAUCUUCCUG |
| siRNA 2192 | 2192 | GGAAGAUAUCAGUCCUAGU | 8546 | ACUAGGACUGAUAUCUUCC |
| siRNA 2193 | 2193 | GAAGAUAUCAGUCCUAGUC | 8547 | GACUAGGACUGAUAUCUUC |
| siRNA 2199 | 2199 | AUCAGUCCUAGUCUUGCUG | 8553 | CAGCAAGACUAGGACUGAU |
| siRNA 2205 | 2205 | CCUAGUCUUGCUGAGCAGC | 8559 | GCUGCUCAGCAAGACUAGG |
| siRNA 2294 | 2294 | GGAGGAACAGCGAGAUUGC | 8648 | GCAAUCUCGCUGUUCCUCC |
| siRNA 2295 | 2295 | GAGGAACAGCGAGAUUGCU | 8649 | AGCAAUCUCGCUGUUCCUC |
| siRNA 2296 | 2296 | AGGAACAGCGAGAUUGCUA | 8650 | UAGCAAUCUCGCUGUUCCU |
| siRNA 2297 | 2297 | GGAACAGCGAGAUUGCUAC | 8651 | GUAGCAAUCUCGCUGUUCC |
| siRNA 2299 | 2299 | AACAGCGAGAUUGCUACCU | 8653 | AGGUAGCAAUCUCGCUGUU |
| siRNA 2301 | 2301 | CAGCGAGAUUGCUACCUGA | 8655 | UCAGGUAGCAAUCUCGCUG |
| siRNA 2302 | 2302 | AGCGAGAUUGCUACCUGAA | 8656 | UUCAGGUAGCAAUCUCGCU |
| siRNA 2303 | 2303 | GCGAGAUUGCUACCUGAAG | 8657 | CUUCAGGUAGCAAUCUCGC |
| siRNA 2304 | 2304 | CGAGAUUGCUACCUGAAGG | 8658 | CCUUCAGGUAGCAAUCUCG |
| siRNA 2314 | 2314 | ACCUGAAGGUGAGCCAAUC | 8668 | GAUUGGCUCACCUUCAGGU |
| siRNA 2324 | 2324 | GAGCCAAUCCAAGGAGCAC | 8678 | GUGCUCCUUGGAUUGGCUC |
| siRNA 2332 | 2332 | CCAAGGAGCACCAGCAGUU | 8686 | AACUGCUGGUGCUCCUUGG |
| siRNA 2340 | 2340 | CACCAGCAGUUUAUCACCU | 8694 | AGGUGAUAAACUGCUGGUG |
| siRNA 2344 | 2344 | AGCAGUUUAUCACCUUCUU | 8698 | AAGAAGGUGAUAAACUGCU |
| siRNA 2345 | 2345 | GCAGUUUAUCACCUUCUUA | 8699 | UAAGAAGGUGAUAAACUGC |
| siRNA 2366 | 2366 | GAGACUCCUUGGGCCUUUG | 8720 | CAAAGGCCCAAGGAGUCUC |
| siRNA 2367 | 2367 | AGACUCCUUGGGCCUUUGC | 8721 | GCAAAGGCCCAAGGAGUCU |
| siRNA 2378 | 2378 | GCCUUUGCUGGAGGCCUAC | 8732 | GUAGGCCUCCAGCAAAGGC |
| siRNA 2407 | 2407 | CCAUCUUUGUUCACAACUU | 8761 | AAGUUGUGAACAAAGAUGG |
| siRNA 2415 | 2415 | GUUCACAACUUCAGUGGUC | 8769 | GACCACUGAAGUUGUGAAC |
| siRNA 2427 | 2427 | AGUGGUCCUGUUCCAGAAC | 8781 | GUUCUGGAACAGGACCACU |
| siRNA 2435 | 2435 | UGUUCCAGAACCUGAGUAU | 8789 | AUACUCAGGUUCUGGAACA |
| siRNA 2436 | 2436 | GUUCCAGAACCUGAGUAUC | 8790 | GAUACUCAGGUUCUGGAAC |
| siRNA 2437 | 2437 | UUCCAGAACCUGAGUAUCU | 8791 | AGAUACUCAGGUUCUGGAA |
| siRNA 2440 | 2440 | CAGAACCUGAGUAUCUGCA | 8794 | UGCAGAUACUCAGGUUCUG |
| siRNA 2449 | 2449 | AGUAUCUGCAAAAGUUGCA | 8803 | UGCAACUUUUGCAGAUACU |
| siRNA 2453 | 2453 | UCUGCAAAAGUUGCACAAA | 8807 | UUUGUGCAACUUUUGCAGA |
| siRNA 2461 | 2461 | AGUUGCACAAAUACCUAAU | 8815 | AUUAGGUAUUUGUGCAACU |
| siRNA 2462 | 2462 | GUUGCACAAAUACCUAAUA | 8816 | UAUUAGGUAUUUGUGCAAC |
| siRNA 2463 | 2463 | UUGCACAAAUACCUAAUAA | 8817 | UUAUUAGGUAUUUGUGCAA |
| siRNA 2464 | 2464 | UGCACAAAUACCUAAUAAC | 8818 | GUUAUUAGGUAUUUGUGCA |
| siRNA 2465 | 2465 | GCACAAAUACCUAAUAACC | 8819 | GGUUAUUAGGUAUUUGUGC |
| siRNA 2466 | 2466 | CACAAAUACCUAAUAACCA | 8820 | UGGUUAUUAGGUAUUUGUG |
| siRNA 2468 | 2468 | CAAAUACCUAAUAACCAGA | 8822 | UCUGGUUAUUAGGUAUUUG |
| siRNA 2486 | 2486 | AACAGAAAGAAAUGUUGCA | 8840 | UGCAACAUUUCUUUCUGUU |
| siRNA 2494 | 2494 | GAAAUGUUGCAGUAUAUGC | 8848 | GCAUAUACUGCAACAUUUC |
| siRNA 2496 | 2496 | AAUGUUGCAGUAUAUGCUG | 8850 | CAGCAUAUACUGCAACAUU |
| siRNA 2498 | 2498 | UGUUGCAGUAUAUGCUGAG | 8852 | CUCAGCAUAUACUGCAACA |
| siRNA 2504 | 2504 | AGUAUAUGCUGAGAGUGCC | 8858 | GGCACUCUCAGCAUAUACU |
| siRNA 2507 | 2507 | AUAUGCUGAGAGUGCCACA | 8861 | UGUGGCACUCUCAGCAUAU |
| siRNA 2511 | 2511 | GCUGAGAGUGCCACAUAUU | 8865 | AAUAUGUGGCACUCUCAGC |
| siRNA 2515 | 2515 | AGAGUGCCACAUAUUGUCU | 8869 | AGACAAUAUGUGGCACUCU |
| siRNA 2518 | 2518 | GUGCCACAUAUUGUCUUGU | 8872 | ACAAGACAAUAUGUGGCAC |
| siRNA 2523 | 2523 | ACAUAUUGUCUUGUGAAGA | 8877 | UCUUCACAAGACAAUAUGU |
| siRNA 2524 | 2524 | CAUAUUGUCUUGUGAAGAA | 8878 | UUCUUCACAAGACAAUAUG |
| siRNA 2557 | 2557 | UUAAGGAUAUUGGGGUUUU | 8911 | AAAACCCCAAUAUCCUUAA |
| siRNA 2561 | 2561 | GGAUAUUGGGGUUUUCAAG | 8915 | CUUGAAAACCCCAAUAUCC |
| siRNA 2564 | 2564 | UAUUGGGGUUUUCAAGGAG | 8918 | CUCCUUGAAAACCCCAAUA |
| siRNA 2574 | 2574 | UUCAAGGAGACCAAACAAA | 8928 | UUUGUUUGGUCUCCUUGAA |
| siRNA 2575 | 2575 | UCAAGGAGACCAAACAAAA | 8929 | UUUUGUUUGGUCUCCUUGA |
| siRNA 2597 | 2597 | AGUGUCUGUUUUAGAACUG | 8951 | CAGUUCUAAAACAGACACU |
| siRNA 2600 | 2600 | GUCUGUUUUAGAACUGAGC | 8954 | GCUCAGUUCUAAAACAGAC |
| siRNA 2603 | 2603 | UGUUUUAGAACUGAGCAGC | 8957 | GCUGCUCAGUUCUAAAACA |
| siRNA 2618 | 2618 | CAGCACUUUUCUACCUCAA | 8972 | UUGAGGUAGAAAAGUGCUG |
| siRNA 2619 | 2619 | AGCACUUUUCUACCUCAAU | 8973 | AUUGAGGUAGAAAAGUGCU |
| siRNA 2620 | 2620 | GCACUUUUCUACCUCAAUG | 8974 | CAUUGAGGUAGAAAAGUGC |
| siRNA 2626 | 2626 | UUCUACCUCAAUGCAACCG | 8980 | CGGUUGCAUUGAGGUAGAA |
| siRNA 2627 | 2627 | UCUACCUCAAUGCAACCGA | 8981 | UCGGUUGCAUUGAGGUAGA |
| siRNA 2628 | 2628 | CUACCUCAAUGCAACCGAC | 8982 | GUCGGUUGCAUUGAGGUAG |
| siRNA 2629 | 2629 | UACCUCAAUGCAACCGACA | 8983 | UGUCGGUUGCAUUGAGGUA |
| siRNA 2630 | 2630 | ACCUCAAUGCAACCGACAA | 8984 | UUGUCGGUUGCAUUGAGGU |
| siRNA 2631 | 2631 | CCUCAAUGCAACCGACAAA | 8985 | UUUGUCGGUUGCAUUGAGG |
| siRNA 2632 | 2632 | CUCAAUGCAACCGACAAAA | 8986 | UUUUGUCGGUUGCAUUGAG |
| siRNA 2633 | 2633 | UCAAUGCAACCGACAAAAA | 8987 | UUUUUGUCGGUUGCAUUGA |
| siRNA 2635 | 2635 | AAUGCAACCGACAAAAACU | 8989 | AGUUUUUGUCGGUUGCAUU |
| siRNA 2637 | 2637 | UGCAACCGACAAAAACUUC | 8991 | GAAGUUUUUGUCGGUUGCA |
| siRNA 2641 | 2641 | ACCGACAAAAACUUCUAGA | 8995 | UCUAGAAGUUUUUGUCGGU |
| siRNA 2642 | 2642 | CCGACAAAAACUUCUAGAA | 8996 | UUCUAGAAGUUUUUGUCGG |
| siRNA 2649 | 2649 | AAACUUCUAGAAUAUAUUC | 9003 | GAAUAUAUUCUAGAAGUUU |
| siRNA 2656 | 2656 | UAGAAUAUAUUCUGAGUUU | 9010 | AAACUCAGAAUAUAUUCUA |
| siRNA 2660 | 2660 | AUAUAUUCUGAGUUUUGUG | 9014 | CACAAAACUCAGAAUAUAU |
| siRNA 2661 | 2661 | UAUAUUCUGAGUUUUGUGG | 9015 | CCACAAAACUCAGAAUAUA |
| siRNA 2674 | 2674 | UUGUGGUGCUGUAGGUAAC | 9028 | GUUACCUACAGCACCACAA |
| siRNA 2676 | 2676 | GUGGUGCUGUAGGUAACGU | 9030 | ACGUUACCUACAGCACCAC |
| siRNA 2678 | 2678 | GGUGCUGUAGGUAACGUGU | 9032 | ACACGUUACCUACAGCACC |
| siRNA 2679 | 2679 | GUGCUGUAGGUAACGUGUG | 9033 | CACACGUUACCUACAGCAC |
| siRNA 2680 | 2680 | UGCUGUAGGUAACGUGUGG | 9034 | CCACACGUUACCUACAGCA |
| siRNA 2681 | 2681 | GCUGUAGGUAACGUGUGGC | 9035 | GCCACACGUUACCUACAGC |
| siRNA 2683 | 2683 | UGUAGGUAACGUGUGGCAC | 9037 | GUGCCACACGUUACCUACA |
| siRNA 2684 | 2684 | GUAGGUAACGUGUGGCACU | 9038 | AGUGCCACACGUUACCUAC |
| siRNA 2692 | 2692 | CGUGUGGCACUGCUGGCAA | 9046 | UUGCCAGCAGUGCCACACG |
| siRNA 2702 | 2702 | UGCUGGCAAAUGAAGGUCA | 9056 | UGACCUUCAUUUGCCAGCA |
| siRNA 2708 | 2708 | CAAAUGAAGGUCAUGAGAU | 9062 | AUCUCAUGACCUUCAUUUG |
| siRNA 2711 | 2711 | AUGAAGGUCAUGAGAUGAG | 9065 | CUCAUCUCAUGACCUUCAU |
| siRNA 2713 | 2713 | GAAGGUCAUGAGAUGAGUU | 9067 | AACUCAUCUCAUGACCUUC |
| siRNA 2725 | 2725 | AUGAGUUCCUUGUAGGUAC | 9079 | GUACCUACAAGGAACUCAU |
| siRNA 2726 | 2726 | UGAGUUCCUUGUAGGUACC | 9080 | GGUACCUACAAGGAACUCA |
| siRNA 2727 | 2727 | GAGUUCCUUGUAGGUACCA | 9081 | UGGUACCUACAAGGAACUC |
| siRNA 2740 | 2740 | GUACCAGCUUCUGGCUCAA | 9094 | UUGAGCCAGAAGCUGGUAC |
| siRNA 2808 | 2808 | CUCCUGGAAGACAAGUGCC | 9162 | GGCACUUGUCUUCCAGGAG |
| siRNA 2816 | 2816 | AGACAAGUGCCUUCUCCCU | 9170 | AGGGAGAAGGCACUUGUCU |
| siRNA 2818 | 2818 | ACAAGUGCCUUCUCCCUCC | 9172 | GGAGGGAGAAGGCACUUGU |
| siRNA 2826 | 2826 | CUUCUCCCUCCAUGGAUCU | 9180 | AGAUCCAUGGAGGGAGAAG |
| siRNA 2829 | 2829 | CUCCCUCCAUGGAUCUGUG | 9183 | CACAGAUCCAUGGAGGGAG |
| siRNA 2834 | 2834 | UCCAUGGAUCUGUGAUCUU | 9188 | AAGAUCACAGAUCCAUGGA |
| siRNA 2855 | 2855 | CAGCUCUGCAUCAACACAG | 9209 | CUGUGUUGAUGCAGAGCUG |
| siRNA 2872 | 2872 | AGCAGCCUGCAGAUAACAC | 9226 | GUGUUAUCUGCAGGCUGCU |
| siRNA 2878 | 2878 | CUGCAGAUAACACUUGGGG | 9232 | CCCCAAGUGUUAUCUGCAG |
| siRNA 2880 | 2880 | GCAGAUAACACUUGGGGGG | 9234 | CCCCCCAAGUGUUAUCUGC |
| siRNA 2883 | 2883 | GAUAACACUUGGGGGGACC | 9237 | GGUCCCCCCAAGUGUUAUC |
| siRNA 2886 | 2886 | AACACUUGGGGGGACCUCA | 9240 | UGAGGUCCCCCCAAGUGUU |
| siRNA 2896 | 2896 | GGGACCUCAGCCUCUAUUC | 9250 | GAAUAGAGGCUGAGGUCCC |
| siRNA 2898 | 2898 | GACCUCAGCCUCUAUUCGC | 9252 | GCGAAUAGAGGCUGAGGUC |
| siRNA 2899 | 2899 | ACCUCAGCCUCUAUUCGCA | 9253 | UGCGAAUAGAGGCUGAGGU |
| siRNA 2905 | 2905 | GCCUCUAUUCGCAACUCAU | 9259 | AUGAGUUGCGAAUAGAGGC |
| siRNA 2906 | 2906 | CCUCUAUUCGCAACUCAUA | 9260 | UAUGAGUUGCGAAUAGAGG |
| siRNA 2907 | 2907 | CUCUAUUCGCAACUCAUAA | 9261 | UUAUGAGUUGCGAAUAGAG |
| siRNA 2909 | 2909 | CUAUUCGCAACUCAUAAUC | 9263 | GAUUAUGAGUUGCGAAUAG |
| siRNA 2910 | 2910 | UAUUCGCAACUCAUAAUCC | 9264 | GGAUUAUGAGUUGCGAAUA |
| siRNA 2911 | 2911 | AUUCGCAACUCAUAAUCCG | 9265 | CGGAUUAUGAGUUGCGAAU |
| siRNA 2912 | 2912 | UUCGCAACUCAUAAUCCGU | 9266 | ACGGAUUAUGAGUUGCGAA |
| siRNA 2913 | 2913 | UCGCAACUCAUAAUCCGUA | 9267 | UACGGAUUAUGAGUUGCGA |
| siRNA 2914 | 2914 | CGCAACUCAUAAUCCGUAG | 9268 | CUACGGAUUAUGAGUUGCG |
| siRNA 2915 | 2915 | GCAACUCAUAAUCCGUAGA | 9269 | UCUACGGAUUAUGAGUUGC |
| siRNA 2916 | 2916 | CAACUCAUAAUCCGUAGAC | 9270 | GUCUACGGAUUAUGAGUUG |
| siRNA 2917 | 2917 | AACUCAUAAUCCGUAGACU | 9271 | AGUCUACGGAUUAUGAGUU |
| siRNA 2918 | 2918 | ACUCAUAAUCCGUAGACUA | 9272 | UAGUCUACGGAUUAUGAGU |
| siRNA 2919 | 2919 | CUCAUAAUCCGUAGACUAC | 9273 | GUAGUCUACGGAUUAUGAG |
| siRNA 2920 | 2920 | UCAUAAUCCGUAGACUACA | 9274 | UGUAGUCUACGGAUUAUGA |
| siRNA 2921 | 2921 | CAUAAUCCGUAGACUACAA | 9275 | UUGUAGUCUACGGAUUAUG |
| siRNA 2922 | 2922 | AUAAUCCGUAGACUACAAG | 9276 | CUUGUAGUCUACGGAUUAU |
| siRNA 2924 | 2924 | AAUCCGUAGACUACAAGAU | 9278 | AUCUUGUAGUCUACGGAUU |
| siRNA 2925 | 2925 | AUCCGUAGACUACAAGAUG | 9279 | CAUCUUGUAGUCUACGGAU |
| siRNA 2926 | 2926 | UCCGUAGACUACAAGAUGA | 9280 | UCAUCUUGUAGUCUACGGA |
| siRNA 2927 | 2927 | CCGUAGACUACAAGAUGAA | 9281 | UUCAUCUUGUAGUCUACGG |
| siRNA 2928 | 2928 | CGUAGACUACAAGAUGAAA | 9282 | UUUCAUCUUGUAGUCUACG |
| siRNA 2931 | 2931 | AGACUACAAGAUGAAAUCU | 9285 | AGAUUUCAUCUUGUAGUCU |
| siRNA 2939 | 2939 | AGAUGAAAUCUCAAUAAAU | 9293 | AUUUAUUGAGAUUUCAUCU |
| siRNA 3003 | 3003 | AGGACUAAUUACUGUGAUG | 9357 | CAUCACAGUAAUUAGUCCU |
| siRNA 3004 | 3004 | GGACUAAUUACUGUGAUGG | 9358 | CCAUCACAGUAAUUAGUCC |
| siRNA 3005 | 3005 | GACUAAUUACUGUGAUGGA | 9359 | UCCAUCACAGUAAUUAGUC |
| siRNA 3007 | 3007 | CUAAUUACUGUGAUGGACA | 9361 | UGUCCAUCACAGUAAUUAG |
| siRNA 3033 | 3033 | GUAGCUGUGUUCUGGAACU | 9387 | AGUUCCAGAACACAGCUAC |
| siRNA 3046 | 3046 | GGAACUGAAUCUUACAUGG | 9400 | CCAUGUAAGAUUCAGUUCC |
| siRNA 3047 | 3047 | GAACUGAAUCUUACAUGGU | 9401 | ACCAUGUAAGAUUCAGUUC |
| siRNA 3048 | 3048 | AACUGAAUCUUACAUGGUA | 9402 | UACCAUGUAAGAUUCAGUU |
| siRNA 3049 | 3049 | ACUGAAUCUUACAUGGUAU | 9403 | AUACCAUGUAAGAUUCAGU |
| siRNA 3052 | 3052 | GAAUCUUACAUGGUAUACU | 9406 | AGUAUACCAUGUAAGAUUC |
| siRNA 3054 | 3054 | AUCUUACAUGGUAUACUUA | 9408 | UAAGUAUACCAUGUAAGAU |
| siRNA 3055 | 3055 | UCUUACAUGGUAUACUUAG | 9409 | CUAAGUAUACCAUGUAAGA |
| siRNA 3056 | 3056 | CUUACAUGGUAUACUUAGU | 9410 | ACUAAGUAUACCAUGUAAG |
| siRNA 3057 | 3057 | UUACAUGGUAUACUUAGUG | 9411 | CACUAAGUAUACCAUGUAA |
| siRNA 3058 | 3058 | UACAUGGUAUACUUAGUGC | 9412 | GCACUAAGUAUACCAUGUA |
| siRNA 3059 | 3059 | ACAUGGUAUACUUAGUGCU | 9413 | AGCACUAAGUAUACCAUGU |
| siRNA 3073 | 3073 | GUGCUGCUGGGUAAUUUGU | 9427 | ACAAAUUACCCAGCAGCAC |
| siRNA 3078 | 3078 | GCUGGGUAAUUUGUUGGUA | 9432 | UACCAACAAAUUACCCAGC |
| siRNA 3080 | 3080 | UGGGUAAUUUGUUGGUAUA | 9434 | UAUACCAACAAAUUACCCA |
| siRNA 3081 | 3081 | GGGUAAUUUGUUGGUAUAU | 9435 | AUAUACCAACAAAUUACCC |
| siRNA 3086 | 3086 | AUUUGUUGGUAUAUUAUCU | 9440 | AGAUAAUAUACCAACAAAU |
| siRNA 3090 | 3090 | GUUGGUAUAUUAUCUGGUU | 9444 | AACCAGAUAAUAUACCAAC |
| siRNA 3093 | 3093 | GGUAUAUUAUCUGGUUAGU | 9447 | ACUAACCAGAUAAUAUACC |
| siRNA 3097 | 3097 | UAUUAUCUGGUUAGUGGUU | 9451 | AACCACUAACCAGAUAAUA |
| siRNA 3100 | 3100 | UAUCUGGUUAGUGGUUAAU | 9454 | AUUAACCACUAACCAGAUA |
| siRNA 3106 | 3106 | GUUAGUGGUUAAUGCUUCC | 9460 | GGAAGCAUUAACCACUAAC |
| siRNA 3111 | 3111 | UGGUUAAUGCUUCCUUUAA | 9465 | UUAAAGGAAGCAUUAACCA |
| siRNA 3131 | 3131 | AAUAAUUGAGUCAUCCAUU | 9485 | AAUGGAUGACUCAAUUAUU |
| siRNA 3159 | 3159 | UCAGUUUUAUCUGUCAAUA | 9513 | UAUUGACAGAUAAAACUGA |
| siRNA 3162 | 3162 | GUUUUAUCUGUCAAUAGUA | 9516 | UACUAUUGACAGAUAAAAC |
| siRNA 3163 | 3163 | UUUUAUCUGUCAAUAGUAG | 9517 | CUACUAUUGACAGAUAAAA |
| siRNA 3164 | 3164 | UUUAUCUGUCAAUAGUAGC | 9518 | GCUACUAUUGACAGAUAAA |
| siRNA 3165 | 3165 | UUAUCUGUCAAUAGUAGCU | 9519 | AGCUACUAUUGACAGAUAA |
| siRNA 3166 | 3166 | UAUCUGUCAAUAGUAGCUA | 9520 | UAGCUACUAUUGACAGAUA |
| siRNA 3168 | 3168 | UCUGUCAAUAGUAGCUACA | 9522 | UGUAGCUACUAUUGACAGA |
| siRNA 3169 | 3169 | CUGUCAAUAGUAGCUACAU | 9523 | AUGUAGCUACUAUUGACAG |
| siRNA 3172 | 3172 | UCAAUAGUAGCUACAUUUU | 9526 | AAAAUGUAGCUACUAUUGA |
| siRNA 3181 | 3181 | GCUACAUUUUUAAUGGGAG | 9535 | CUCCCAUUAAAAAUGUAGC |
| siRNA 3185 | 3185 | CAUUUUUAAUGGGAGCACC | 9539 | GGUGCUCCCAUUAAAAAUG |
| siRNA 3189 | 3189 | UUUAAUGGGAGCACCUUUU | 9543 | AAAAGGUGCUCCCAUUAAA |
| siRNA 3190 | 3190 | UUAAUGGGAGCACCUUUUA | 9544 | UAAAAGGUGCUCCCAUUAA |
| siRNA 3191 | 3191 | UAAUGGGAGCACCUUUUAU | 9545 | AUAAAAGGUGCUCCCAUUA |
| siRNA 3192 | 3192 | AAUGGGAGCACCUUUUAUC | 9546 | GAUAAAAGGUGCUCCCAUU |
| siRNA 3194 | 3194 | UGGGAGCACCUUUUAUCCC | 9548 | GGGAUAAAAGGUGCUCCCA |
| siRNA 3195 | 3195 | GGGAGCACCUUUUAUCCCA | 9549 | UGGGAUAAAAGGUGCUCCC |
| siRNA 3205 | 3205 | UUUAUCCCAAAGUGCUUUA | 9559 | UAAAGCACUUUGGGAUAAA |
| siRNA 3219 | 3219 | CUUUAUAAAUUGAGUGGAC | 9573 | GUCCACUCAAUUUAUAAAG |
| siRNA 3233 | 3233 | UGGACUGAUAUAUAUCACA | 9587 | UGUGAUAUAUAUCAGUCCA |
| siRNA 3234 | 3234 | GGACUGAUAUAUAUCACAC | 9588 | GUGUGAUAUAUAUCAGUCC |
| siRNA 3238 | 3238 | UGAUAUAUAUCACACCCAG | 9592 | CUGGGUGUGAUAUAUAUCA |
| siRNA 3239 | 3239 | GAUAUAUAUCACACCCAGG | 9593 | CCUGGGUGUGAUAUAUAUC |
| siRNA 3241 | 3241 | UAUAUAUCACACCCAGGUA | 9595 | UACCUGGGUGUGAUAUAUA |
| siRNA 3244 | 3244 | AUAUCACACCCAGGUAUCA | 9598 | UGAUACCUGGGUGUGAUAU |
| siRNA 3246 | 3246 | AUCACACCCAGGUAUCACU | 9600 | AGUGAUACCUGGGUGUGAU |
| siRNA 3247 | 3247 | UCACACCCAGGUAUCACUG | 9601 | CAGUGAUACCUGGGUGUGA |
| siRNA 3265 | 3265 | GUGCUGUCCUUUGCUGUCA | 9619 | UGACAGCAAAGGACAGCAC |
| siRNA 3268 | 3268 | CUGUCCUUUGCUGUCAGAU | 9622 | AUCUGACAGCAAAGGACAG |
| siRNA 3269 | 3269 | UGUCCUUUGCUGUCAGAUU | 9623 | AAUCUGACAGCAAAGGACA |
| siRNA 3274 | 3274 | UUUGCUGUCAGAUUUAGAA | 9628 | UUCUAAAUCUGACAGCAAA |
| siRNA 3305 | 3305 | GCUAUGUGAAAACAGACAA | 9659 | UUGUCUGUUUUCACAUAGC |
| siRNA 3316 | 3316 | ACAGACAAUAUUAGUUUAG | 9670 | CUAAACUAAUAUUGUCUGU |
| siRNA 3317 | 3317 | CAGACAAUAUUAGUUUAGG | 9671 | CCUAAACUAAUAUUGUCUG |
| siRNA 3318 | 3318 | AGACAAUAUUAGUUUAGGU | 9672 | ACCUAAACUAAUAUUGUCU |
| siRNA 3321 | 3321 | CAAUAUUAGUUUAGGUCGG | 9675 | CCGACCUAAACUAAUAUUG |
| siRNA 3322 | 3322 | AAUAUUAGUUUAGGUCGGG | 9676 | CCCGACCUAAACUAAUAUU |
| siRNA 3323 | 3323 | AUAUUAGUUUAGGUCGGGA | 9677 | UCCCGACCUAAACUAAUAU |
| siRNA 3324 | 3324 | UAUUAGUUUAGGUCGGGAA | 9678 | UUCCCGACCUAAACUAAUA |
| siRNA 3325 | 3325 | AUUAGUUUAGGUCGGGAAC | 9679 | GUUCCCGACCUAAACUAAU |
| siRNA 3326 | 3326 | UUAGUUUAGGUCGGGAACU | 9680 | AGUUCCCGACCUAAACUAA |
| siRNA 3327 | 3327 | UAGUUUAGGUCGGGAACUG | 9681 | CAGUUCCCGACCUAAACUA |
| siRNA 3330 | 3330 | UUUAGGUCGGGAACUGAGA | 9684 | UCUCAGUUCCCGACCUAAA |
| siRNA 3331 | 3331 | UUAGGUCGGGAACUGAGAU | 9685 | AUCUCAGUUCCCGACCUAA |
| siRNA 3333 | 3333 | AGGUCGGGAACUGAGAUAU | 9687 | AUAUCUCAGUUCCCGACCU |
| siRNA 3334 | 3334 | GGUCGGGAACUGAGAUAUU | 9688 | AAUAUCUCAGUUCCCGACC |
| siRNA 3338 | 3338 | GGGAACUGAGAUAUUGUAA | 9692 | UUACAAUAUCUCAGUUCCC |
| siRNA 3341 | 3341 | AACUGAGAUAUUGUAAUCA | 9695 | UGAUUACAAUAUCUCAGUU |
| siRNA 3358 | 3358 | CAAAUAGUUAACAUCAGGA | 9712 | UCCUGAUGUUAACUAUUUG |
| siRNA 3365 | 3365 | UUAACAUCAGGAAGUUAAU | 9719 | AUUAACUUCCUGAUGUUAA |
| siRNA 3370 | 3370 | AUCAGGAAGUUAAUUUGGC | 9724 | GCCAAAUUAACUUCCUGAU |
| siRNA 3374 | 3374 | GGAAGUUAAUUUGGCUGGC | 9728 | GCCAGCCAAAUUAACUUCC |
| siRNA 3380 | 3380 | UAAUUUGGCUGGCAAAAUU | 9734 | AAUUUUGCCAGCCAAAUUA |
| siRNA 3382 | 3382 | AUUUGGCUGGCAAAAUUCU | 9736 | AGAAUUUUGCCAGCCAAAU |
| siRNA 3383 | 3383 | UUUGGCUGGCAAAAUUCUA | 9737 | UAGAAUUUUGCCAGCCAAA |
| siRNA 3388 | 3388 | CUGGCAAAAUUCUAGGGAA | 9742 | UUCCCUAGAAUUUUGCCAG |
| siRNA 3404 | 3404 | GAAACUUGGCCAGAAAACU | 9758 | AGUUUUCUGGCCAAGUUUC |
| siRNA 3406 | 3406 | AACUUGGCCAGAAAACUGG | 9760 | CCAGUUUUCUGGCCAAGUU |
| siRNA 3409 | 3409 | UUGGCCAGAAAACUGGUGU | 9763 | ACACCAGUUUUCUGGCCAA |
| siRNA 3411 | 3411 | GGCCAGAAAACUGGUGUUG | 9765 | CAACACCAGUUUUCUGGCC |
| siRNA 3425 | 3425 | UGUUGAAGGCUUUUGCUCA | 9779 | UGAGCAAAAGCCUUCAACA |
| siRNA 3429 | 3429 | GAAGGCUUUUGCUCAUAUA | 9783 | UAUAUGAGCAAAAGCCUUC |
| siRNA 3431 | 3431 | AGGCUUUUGCUCAUAUAAA | 9785 | UUUAUAUGAGCAAAAGCCU |
| siRNA 3434 | 3434 | CUUUUGCUCAUAUAAACAA | 9788 | UUGUUUAUAUGAGCAAAAG |
| siRNA 3439 | 3439 | GCUCAUAUAAACAAGUGCC | 9793 | GGCACUUGUUUAUAUGAGC |
| siRNA 3440 | 3440 | CUCAUAUAAACAAGUGCCA | 9794 | UGGCACUUGUUUAUAUGAG |
| siRNA 3445 | 3445 | AUAAACAAGUGCCAUUGAG | 9799 | CUCAAUGGCACUUGUUUAU |
| siRNA 3446 | 3446 | UAAACAAGUGCCAUUGAGU | 9800 | ACUCAAUGGCACUUGUUUA |
| siRNA 3447 | 3447 | AAACAAGUGCCAUUGAGUU | 9801 | AACUCAAUGGCACUUGUUU |
| siRNA 3452 | 3452 | AGUGCCAUUGAGUUUCAAA | 9806 | UUUGAAACUCAAUGGCACU |
| siRNA 3454 | 3454 | UGCCAUUGAGUUUCAAAUG | 9808 | CAUUUGAAACUCAAUGGCA |
| siRNA 3464 | 3464 | UUUCAAAUGACCAGCAAAU | 9818 | AUUUGCUGGUCAUUUGAAA |
| siRNA 3465 | 3465 | UUCAAAUGACCAGCAAAUA | 9819 | UAUUUGCUGGUCAUUUGAA |
| siRNA 3466 | 3466 | UCAAAUGACCAGCAAAUAU | 9820 | AUAUUUGCUGGUCAUUUGA |
| siRNA 3467 | 3467 | CAAAUGACCAGCAAAUAUA | 9821 | UAUAUUUGCUGGUCAUUUG |
| siRNA 3468 | 3468 | AAAUGACCAGCAAAUAUAU | 9822 | AUAUAUUUGCUGGUCAUUU |
| siRNA 3470 | 3470 | AUGACCAGCAAAUAUAUUU | 9824 | AAAUAUAUUUGCUGGUCAU |
| siRNA 3480 | 3480 | AAUAUAUUUAGAACCCUUC | 9834 | GAAGGGUUCUAAAUAUAUU |
| siRNA 3481 | 3481 | AUAUAUUUAGAACCCUUCC | 9835 | GGAAGGGUUCUAAAUAUAU |
| siRNA 3487 | 3487 | UUAGAACCCUUCCUGUUUU | 9841 | AAAACAGGAAGGGUUCUAA |
| siRNA 3496 | 3496 | UUCCUGUUUUAUGUCUGUA | 9850 | UACAGACAUAAAACAGGAA |
| siRNA 3498 | 3498 | CCUGUUUUAUGUCUGUACC | 9852 | GGUACAGACAUAAAACAGG |
| siRNA 3503 | 3503 | UUUAUGUCUGUACCUCGUC | 9857 | GACGAGGUACAGACAUAAA |
| siRNA 3504 | 3504 | UUAUGUCUGUACCUCGUCC | 9858 | GGACGAGGUACAGACAUAA |
| siRNA 3508 | 3508 | GUCUGUACCUCGUCCACCC | 9862 | GGGUGGACGAGGUACAGAC |
| siRNA 3516 | 3516 | CUCGUCCACCCCUCAGGUA | 9870 | UACCUGAGGGGUGGACGAG |
| siRNA 3518 | 3518 | CGUCCACCCCUCAGGUAAU | 9872 | AUUACCUGAGGGGUGGACG |
| siRNA 3520 | 3520 | UCCACCCCUCAGGUAAUAC | 9874 | GUAUUACCUGAGGGGUGGA |
| siRNA 3522 | 3522 | CACCCCUCAGGUAAUACCU | 9876 | AGGUAUUACCUGAGGGGUG |
| siRNA 3523 | 3523 | ACCCCUCAGGUAAUACCUG | 9877 | CAGGUAUUACCUGAGGGGU |
| siRNA 3524 | 3524 | CCCCUCAGGUAAUACCUGC | 9878 | GCAGGUAUUACCUGAGGGG |
| siRNA 3526 | 3526 | CCUCAGGUAAUACCUGCCU | 9880 | AGGCAGGUAUUACCUGAGG |
| siRNA 3537 | 3537 | ACCUGCCUCUCACAGGUAC | 9891 | GUACCUGUGAGAGGCAGGU |
| siRNA 3556 | 3556 | AGCUGUUUCUUGGAAAUCC | 9910 | GGAUUUCCAAGAAACAGCU |
| siRNA 3559 | 3559 | UGUUUCUUGGAAAUCCUCC | 9913 | GGAGGAUUUCCAAGAAACA |
| siRNA 3563 | 3563 | UCUUGGAAAUCCUCCAACC | 9917 | GGUUGGAGGAUUUCCAAGA |
| siRNA 3571 | 3571 | AUCCUCCAACCAAAUAGCA | 9925 | UGCUAUUUGGUUGGAGGAU |
| siRNA 3572 | 3572 | UCCUCCAACCAAAUAGCAG | 9926 | CUGCUAUUUGGUUGGAGGA |
| siRNA 3574 | 3574 | CUCCAACCAAAUAGCAGUU | 9928 | AACUGCUAUUUGGUUGGAG |
| siRNA 3575 | 3575 | UCCAACCAAAUAGCAGUUU | 9929 | AAACUGCUAUUUGGUUGGA |
| siRNA 3576 | 3576 | CCAACCAAAUAGCAGUUUU | 9930 | AAAACUGCUAUUUGGUUGG |
| siRNA 3583 | 3583 | AAUAGCAGUUUUCCUAACU | 9937 | AGUUAGGAAAACUGCUAUU |
| siRNA 3584 | 3584 | AUAGCAGUUUUCCUAACUU | 9938 | AAGUUAGGAAAACUGCUAU |
| siRNA 3591 | 3591 | UUUUCCUAACUUGAUUAGC | 9945 | GCUAAUCAAGUUAGGAAAA |
| siRNA 3593 | 3593 | UUCCUAACUUGAUUAGCUU | 9947 | AAGCUAAUCAAGUUAGGAA |
| siRNA 3594 | 3594 | UCCUAACUUGAUUAGCUUG | 9948 | CAAGCUAAUCAAGUUAGGA |
| siRNA 3595 | 3595 | CCUAACUUGAUUAGCUUGA | 9949 | UCAAGCUAAUCAAGUUAGG |
| siRNA 3598 | 3598 | AACUUGAUUAGCUUGAGCU | 9952 | AGCUCAAGCUAAUCAAGUU |
| siRNA 3599 | 3599 | ACUUGAUUAGCUUGAGCUG | 9953 | CAGCUCAAGCUAAUCAAGU |
| siRNA 3606 | 3606 | UAGCUUGAGCUGACAGACU | 9960 | AGUCUGUCAGCUCAAGCUA |
| siRNA 3614 | 3614 | GCUGACAGACUGUUAGAAU | 9968 | AUUCUAACAGUCUGUCAGC |
| siRNA 3616 | 3616 | UGACAGACUGUUAGAAUAC | 9970 | GUAUUCUAACAGUCUGUCA |
| siRNA 3620 | 3620 | AGACUGUUAGAAUACAGUU | 9974 | AACUGUAUUCUAACAGUCU |
| siRNA 3646 | 3646 | CACAGCUGAUGAGGGCUUU | 10000 | AAAGCCCUCAUCAGCUGUG |
| siRNA 3647 | 3647 | ACAGCUGAUGAGGGCUUUC | 10001 | GAAAGCCCUCAUCAGCUGU |
| siRNA 3656 | 3656 | GAGGGCUUUCUGUACUGCA | 10010 | UGCAGUACAGAAAGCCCUC |
| siRNA 3659 | 3659 | GGCUUUCUGUACUGCACAC | 10013 | GUGUGCAGUACAGAAAGCC |
| siRNA 3662 | 3662 | UUUCUGUACUGCACACAGA | 10016 | UCUGUGUGCAGUACAGAAA |
| siRNA 3667 | 3667 | GUACUGCACACAGAUUGUG | 10021 | CACAAUCUGUGUGCAGUAC |
| siRNA 3670 | 3670 | CUGCACACAGAUUGUGUAC | 10024 | GUACACAAUCUGUGUGCAG |
| siRNA 3671 | 3671 | UGCACACAGAUUGUGUACU | 10025 | AGUACACAAUCUGUGUGCA |
| siRNA 3672 | 3672 | GCACACAGAUUGUGUACUG | 10026 | CAGUACACAAUCUGUGUGC |
| siRNA 3674 | 3674 | ACACAGAUUGUGUACUGCA | 10028 | UGCAGUACACAAUCUGUGU |
| siRNA 3677 | 3677 | CAGAUUGUGUACUGCACCC | 10031 | GGGUGCAGUACACAAUCUG |
| siRNA 3682 | 3682 | UGUGUACUGCACCCCAGUC | 10036 | GACUGGGGUGCAGUACACA |
| siRNA 3683 | 3683 | GUGUACUGCACCCCAGUCC | 10037 | GGACUGGGGUGCAGUACAC |
| siRNA 3684 | 3684 | UGUACUGCACCCCAGUCCA | 10038 | UGGACUGGGGUGCAGUACA |
| siRNA 3690 | 3690 | GCACCCCAGUCCAGGUGAC | 10044 | GUCACCUGGACUGGGGUGC |
| siRNA 3699 | 3699 | UCCAGGUGACUGGUACCCA | 10053 | UGGGUACCAGUCACCUGGA |
| siRNA 3701 | 3701 | CAGGUGACUGGUACCCACU | 10055 | AGUGGGUACCAGUCACCUG |
| siRNA 3704 | 3704 | GUGACUGGUACCCACUCGA | 10058 | UCGAGUGGGUACCAGUCAC |
| siRNA 3709 | 3709 | UGGUACCCACUCGAGUUGU | 10063 | ACAACUCGAGUGGGUACCA |
| siRNA 3710 | 3710 | GGUACCCACUCGAGUUGUG | 10064 | CACAACUCGAGUGGGUACC |
| siRNA 3711 | 3711 | GUACCCACUCGAGUUGUGC | 10065 | GCACAACUCGAGUGGGUAC |
| siRNA 3714 | 3714 | CCCACUCGAGUUGUGCCGU | 10068 | ACGGCACAACUCGAGUGGG |
| siRNA 3715 | 3715 | CCACUCGAGUUGUGCCGUG | 10069 | CACGGCACAACUCGAGUGG |
| siRNA 3716 | 3716 | CACUCGAGUUGUGCCGUGC | 10070 | GCACGGCACAACUCGAGUG |
| siRNA 3717 | 3717 | ACUCGAGUUGUGCCGUGCA | 10071 | UGCACGGCACAACUCGAGU |
| siRNA 3718 | 3718 | CUCGAGUUGUGCCGUGCAC | 10072 | GUGCACGGCACAACUCGAG |
| siRNA 3719 | 3719 | UCGAGUUGUGCCGUGCACA | 10073 | UGUGCACGGCACAACUCGA |
| siRNA 3731 | 3731 | GUGCACAACCUGUCCAGUA | 10085 | UACUGGACAGGUUGUGCAC |
| siRNA 3733 | 3733 | GCACAACCUGUCCAGUAUA | 10087 | UAUACUGGACAGGUUGUGC |
| siRNA 3734 | 3734 | CACAACCUGUCCAGUAUAU | 10088 | AUAUACUGGACAGGUUGUG |
| siRNA 3740 | 3740 | CUGUCCAGUAUAUGCAUGU | 10094 | ACAUGCAUAUACUGGACAG |
| siRNA 3746 | 3746 | AGUAUAUGCAUGUGGUGGC | 10100 | GCCACCACAUGCAUAUACU |
| siRNA 3749 | 3749 | AUAUGCAUGUGGUGGCCCU | 10103 | AGGGCCACCACAUGCAUAU |
| siRNA 3752 | 3752 | UGCAUGUGGUGGCCCUACU | 10106 | AGUAGGGCCACCACAUGCA |
| siRNA 3753 | 3753 | GCAUGUGGUGGCCCUACUG | 10107 | CAGUAGGGCCACCACAUGC |
| siRNA 3757 | 3757 | GUGGUGGCCCUACUGACUG | 10111 | CAGUCAGUAGGGCCACCAC |
| siRNA 3758 | 3758 | UGGUGGCCCUACUGACUGG | 10112 | CCAGUCAGUAGGGCCACCA |
| siRNA 3759 | 3759 | GGUGGCCCUACUGACUGGU | 10113 | ACCAGUCAGUAGGGCCACC |
| siRNA 3761 | 3761 | UGGCCCUACUGACUGGUAA | 10115 | UUACCAGUCAGUAGGGCCA |
| siRNA 3763 | 3763 | GCCCUACUGACUGGUAAUG | 10117 | CAUUACCAGUCAGUAGGGC |
| siRNA 3765 | 3765 | CCUACUGACUGGUAAUGGU | 10119 | ACCAUUACCAGUCAGUAGG |
| siRNA 3766 | 3766 | CUACUGACUGGUAAUGGUU | 10120 | AACCAUUACCAGUCAGUAG |
| siRNA 3771 | 3771 | GACUGGUAAUGGUUAGAGG | 10125 | CCUCUAACCAUUACCAGUC |
| siRNA 3773 | 3773 | CUGGUAAUGGUUAGAGGCA | 10127 | UGCCUCUAACCAUUACCAG |
| siRNA 3780 | 3780 | UGGUUAGAGGCAUUUAUGG | 10134 | CCAUAAAUGCCUCUAACCA |
| siRNA 3808 | 3808 | UUUGAGGAAAAACCAUGAC | 10162 | GUCAUGGUUUUUCCUCAAA |
| siRNA 3810 | 3810 | UGAGGAAAAACCAUGACUU | 10164 | AAGUCAUGGUUUUUCCUCA |
| siRNA 3829 | 3829 | UUAACAAAUUUUUAUGGGU | 10183 | ACCCAUAAAAAUUUGUUAA |
| siRNA 3832 | 3832 | ACAAAUUUUUAUGGGUUAU | 10186 | AUAACCCAUAAAAAUUUGU |
| siRNA 3836 | 3836 | AUUUUUAUGGGUUAUAUGC | 10190 | GCAUAUAACCCAUAAAAAU |
| siRNA 3840 | 3840 | UUAUGGGUUAUAUGCCUAA | 10194 | UUAGGCAUAUAACCCAUAA |
| siRNA 3842 | 3842 | AUGGGUUAUAUGCCUAAAC | 10196 | GUUUAGGCAUAUAACCCAU |
| siRNA 3845 | 3845 | GGUUAUAUGCCUAAACCCU | 10199 | AGGGUUUAGGCAUAUAACC |
| siRNA 3848 | 3848 | UAUAUGCCUAAACCCUUAU | 10202 | AUAAGGGUUUAGGCAUAUA |
| siRNA 3849 | 3849 | AUAUGCCUAAACCCUUAUG | 10203 | CAUAAGGGUUUAGGCAUAU |
| siRNA 3853 | 3853 | GCCUAAACCCUUAUGCCAC | 10207 | GUGGCAUAAGGGUUUAGGC |
| siRNA 3854 | 3854 | CCUAAACCCUUAUGCCACA | 10208 | UGUGGCAUAAGGGUUUAGG |
| siRNA 3859 | 3859 | ACCCUUAUGCCACAUAGUG | 10213 | CACUAUGUGGCAUAAGGGU |
| siRNA 3861 | 3861 | CCUUAUGCCACAUAGUGGU | 10215 | ACCACUAUGUGGCAUAAGG |
| siRNA 3863 | 3863 | UUAUGCCACAUAGUGGUAA | 10217 | UUACCACUAUGUGGCAUAA |
| siRNA 3864 | 3864 | UAUGCCACAUAGUGGUAAA | 10218 | UUUACCACUAUGUGGCAUA |
| siRNA 3865 | 3865 | AUGCCACAUAGUGGUAAAU | 10219 | AUUUACCACUAUGUGGCAU |
| siRNA 3866 | 3866 | UGCCACAUAGUGGUAAAUA | 10220 | UAUUUACCACUAUGUGGCA |
| siRNA 3871 | 3871 | CAUAGUGGUAAAUAAUUAU | 10225 | AUAAUUAUUUACCACUAUG |
| siRNA 3874 | 3874 | AGUGGUAAAUAAUUAUGAA | 10228 | UUCAUAAUUAUUUACCACU |
| siRNA 3896 | 3896 | UGGUCUGUUCAUAAUUGGU | 10250 | ACCAAUUAUGAACAGACCA |
| siRNA 3898 | 3898 | GUCUGUUCAUAAUUGGUAG | 10252 | CUACCAAUUAUGAACAGAC |
| siRNA 3905 | 3905 | CAUAAUUGGUAGGUGCCUU | 10259 | AAGGCACCUACCAAUUAUG |
| siRNA 3926 | 3926 | GUGAGCAGGGAGCAUAAUU | 10280 | AAUUAUGCUCCCUGCUCAC |
| siRNA 3931 | 3931 | CAGGGAGCAUAAUUAUUGG | 10285 | CCAAUAAUUAUGCUCCCUG |
| siRNA 3933 | 3933 | GGGAGCAUAAUUAUUGGUU | 10287 | AACCAAUAAUUAUGCUCCC |
| siRNA 3934 | 3934 | GGAGCAUAAUUAUUGGUUU | 10288 | AAACCAAUAAUUAUGCUCC |
| siRNA 3986 | 3986 | UGUAAUGUUAAAACGUUUU | 10340 | AAAACGUUUUAACAUUACA |
| siRNA 3991 | 3991 | UGUUAAAACGUUUUCUAAC | 10345 | GUUAGAAAACGUUUUAACA |
| siRNA 3995 | 3995 | AAAACGUUUUCUAACAGUU | 10349 | AACUGUUAGAAAACGUUUU |
| siRNA 3996 | 3996 | AAACGUUUUCUAACAGUUU | 10350 | AAACUGUUAGAAAACGUUU |
| siRNA 3997 | 3997 | AACGUUUUCUAACAGUUUA | 10351 | UAAACUGUUAGAAAACGUU |
| siRNA 3998 | 3998 | ACGUUUUCUAACAGUUUAC | 10352 | GUAAACUGUUAGAAAACGU |
| siRNA 4004 | 4004 | UCUAACAGUUUACUGUUGC | 10358 | GCAACAGUAAACUGUUAGA |
| siRNA 4005 | 4005 | CUAACAGUUUACUGUUGCU | 10359 | AGCAACAGUAAACUGUUAG |
| siRNA 4010 | 4010 | AGUUUACUGUUGCUUAUCU | 10364 | AGAUAAGCAACAGUAAACU |
| siRNA 4011 | 4011 | GUUUACUGUUGCUUAUCUC | 10365 | GAGAUAAGCAACAGUAAAC |
| siRNA 4016 | 4016 | CUGUUGCUUAUCUCCAAGA | 10370 | UCUUGGAGAUAAGCAACAG |
| siRNA 4019 | 4019 | UUGCUUAUCUCCAAGAUAU | 10373 | AUAUCUUGGAGAUAAGCAA |
| siRNA 4022 | 4022 | CUUAUCUCCAAGAUAUUAU | 10376 | AUAAUAUCUUGGAGAUAAG |
| siRNA 4024 | 4024 | UAUCUCCAAGAUAUUAUGG | 10378 | CCAUAAUAUCUUGGAGAUA |
| siRNA 4025 | 4025 | AUCUCCAAGAUAUUAUGGA | 10379 | UCCAUAAUAUCUUGGAGAU |
| siRNA 4053 | 4053 | UUUUCCAGAUGAGUGUUAC | 10407 | GUAACACUCAUCUGGAAAA |
| siRNA 4057 | 4057 | CCAGAUGAGUGUUACAUAG | 10411 | CUAUGUAACACUCAUCUGG |
| siRNA 4058 | 4058 | CAGAUGAGUGUUACAUAGA | 10412 | UCUAUGUAACACUCAUCUG |
| siRNA 4073 | 4073 | UAGAUUCUUUGAAUUUAGU | 10427 | ACUAAAUUCAAAGAAUCUA |
| siRNA 4087 | 4087 | UUAGUAUAAAAGUACUGAG | 10441 | CUCAGUACUUUUAUACUAA |
| siRNA 4091 | 4091 | UAUAAAAGUACUGAGAAUU | 10445 | AAUUCUCAGUACUUUUAUA |
| siRNA 4095 | 4095 | AAAGUACUGAGAAUUAAGU | 10449 | ACUUAAUUCUCAGUACUUU |
| siRNA 4099 | 4099 | UACUGAGAAUUAAGUUUGU | 10453 | ACAAACUUAAUUCUCAGUA |
| siRNA 4101 | 4101 | CUGAGAAUUAAGUUUGUAC | 10455 | GUACAAACUUAAUUCUCAG |
| siRNA 4106 | 4106 | AAUUAAGUUUGUACUUCCA | 10460 | UGGAAGUACAAACUUAAUU |
| siRNA 4115 | 4115 | UGUACUUCCAUAAGCUUGG | 10469 | CCAAGCUUAUGGAAGUACA |
| siRNA 4116 | 4116 | GUACUUCCAUAAGCUUGGA | 10470 | UCCAAGCUUAUGGAAGUAC |
| siRNA 4142 | 4142 | CACUGAUAGUAUCUCAUGA | 10496 | UCAUGAGAUACUAUCAGUG |
| siRNA 4146 | 4146 | GAUAGUAUCUCAUGAGUAA | 10500 | UUACUCAUGAGAUACUAUC |
| siRNA 4148 | 4148 | UAGUAUCUCAUGAGUAAUG | 10502 | CAUUACUCAUGAGAUACUA |
| siRNA 4149 | 4149 | AGUAUCUCAUGAGUAAUGU | 10503 | ACAUUACUCAUGAGAUACU |
| siRNA 4151 | 4151 | UAUCUCAUGAGUAAUGUGU | 10505 | ACACAUUACUCAUGAGAUA |
| siRNA 4179 | 4179 | GAGGGAGGGAUGCUGAUUG | 10533 | CAAUCAGCAUCCCUCCCUC |
| siRNA 4180 | 4180 | AGGGAGGGAUGCUGAUUGA | 10534 | UCAAUCAGCAUCCCUCCCU |
| siRNA 4186 | 4186 | GGAUGCUGAUUGAUAUUUC | 10540 | GAAAUAUCAAUCAGCAUCC |
| siRNA 4197 | 4197 | GAUAUUUCACAUUGUAUGA | 10551 | UCAUACAAUGUGAAAUAUC |
| siRNA 4207 | 4207 | AUUGUAUGAAAUACCAUGU | 10561 | ACAUGGUAUUUCAUACAAU |
| siRNA 4211 | 4211 | UAUGAAAUACCAUGUUUGA | 10565 | UCAAACAUGGUAUUUCAUA |
| siRNA 4218 | 4218 | UACCAUGUUUGAAACUCAU | 10572 | AUGAGUUUCAAACAUGGUA |
| siRNA 4220 | 4220 | CCAUGUUUGAAACUCAUAG | 10574 | CUAUGAGUUUCAAACAUGG |
| siRNA 4227 | 4227 | UGAAACUCAUAGCAAUAAU | 10581 | AUUAUUGCUAUGAGUUUCA |
| siRNA 4256 | 4256 | UGUGAUCCCUCUCAAGUUC | 10610 | GAACUUGAGAGGGAUCACA |
| siRNA 4262 | 4262 | CCCUCUCAAGUUCUGCAUU | 10616 | AAUGCAGAACUUGAGAGGG |
| siRNA 4265 | 4265 | UCUCAAGUUCUGCAUUUAA | 10619 | UUAAAUGCAGAACUUGAGA |
| siRNA 4319 | 4319 | AUGAAGUCAUUGUCAGUUG | 10673 | CAACUGACAAUGACUUCAU |
| siRNA 4324 | 4324 | GUCAUUGUCAGUUGUAGUA | 10678 | UACUACAACUGACAAUGAC |
| siRNA 4325 | 4325 | UCAUUGUCAGUUGUAGUAG | 10679 | CUACUACAACUGACAAUGA |
| siRNA 4327 | 4327 | AUUGUCAGUUGUAGUAGCU | 10681 | AGCUACUACAACUGACAAU |
| siRNA 4330 | 4330 | GUCAGUUGUAGUAGCUCUG | 10684 | CAGAGCUACUACAACUGAC |
| siRNA 4333 | 4333 | AGUUGUAGUAGCUCUGAUG | 10687 | CAUCAGAGCUACUACAACU |
| siRNA 4334 | 4334 | GUUGUAGUAGCUCUGAUGU | 10688 | ACAUCAGAGCUACUACAAC |
| siRNA 4335 | 4335 | UUGUAGUAGCUCUGAUGUU | 10689 | AACAUCAGAGCUACUACAA |
| siRNA 4336 | 4336 | UGUAGUAGCUCUGAUGUUG | 10690 | CAACAUCAGAGCUACUACA |
| siRNA 4361 | 4361 | AUAUCAUGUUUUAGCAUUC | 10715 | GAAUGCUAAAACAUGAUAU |
| siRNA 4377 | 4377 | UUCCAUUUUACUGACUAGG | 10731 | CCUAGUCAGUAAAAUGGAA |
| siRNA 4381 | 4381 | AUUUUACUGACUAGGGUAG | 10735 | CUACCCUAGUCAGUAAAAU |
| siRNA 4383 | 4383 | UUUACUGACUAGGGUAGAA | 10737 | UUCUACCCUAGUCAGUAAA |
| siRNA 4384 | 4384 | UUACUGACUAGGGUAGAAG | 10738 | CUUCUACCCUAGUCAGUAA |
| siRNA 4385 | 4385 | UACUGACUAGGGUAGAAGA | 10739 | UCUUCUACCCUAGUCAGUA |
| siRNA 4389 | 4389 | GACUAGGGUAGAAGAACAC | 10743 | GUGUUCUUCUACCCUAGUC |
| siRNA 4400 | 4400 | AAGAACACUUUUCUUGGCU | 10754 | AGCCAAGAAAAGUGUUCUU |
| siRNA 4401 | 4401 | AGAACACUUUUCUUGGCUA | 10755 | UAGCCAAGAAAAGUGUUCU |
| siRNA 4403 | 4403 | AACACUUUUCUUGGCUACA | 10757 | UGUAGCCAAGAAAAGUGUU |
| siRNA 4418 | 4418 | UACAUUUGGAGGAUACCCA | 10772 | UGGGUAUCCUCCAAAUGUA |
| siRNA 4419 | 4419 | ACAUUUGGAGGAUACCCAG | 10773 | CUGGGUAUCCUCCAAAUGU |
| siRNA 4428 | 4428 | GGAUACCCAGGGAGUCUUG | 10782 | CAAGACUCCCUGGGUAUCC |
| siRNA 4429 | 4429 | GAUACCCAGGGAGUCUUGG | 10783 | CCAAGACUCCCUGGGUAUC |
| siRNA 4430 | 4430 | AUACCCAGGGAGUCUUGGG | 10784 | CCCAAGACUCCCUGGGUAU |
| siRNA 4437 | 4437 | GGGAGUCUUGGGUGUUCCU | 10791 | AGGAACACCCAAGACUCCC |
| siRNA 4441 | 4441 | GUCUUGGGUGUUCCUUAUC | 10795 | GAUAAGGAACACCCAAGAC |
| siRNA 4448 | 4448 | GUGUUCCUUAUCUGGGGAA | 10802 | UUCCCCAGAUAAGGAACAC |
| siRNA 4467 | 4467 | GCAAACAUUUCACUAGUCU | 10821 | AGACUAGUGAAAUGUUUGC |
| siRNA 4468 | 4468 | CAAACAUUUCACUAGUCUC | 10822 | GAGACUAGUGAAAUGUUUG |
| siRNA 4478 | 4478 | ACUAGUCUCUUUUUUUCAU | 10832 | AUGAAAAAAAGAGACUAGU |
| siRNA 4479 | 4479 | CUAGUCUCUUUUUUUCAUC | 10833 | GAUGAAAAAAAGAGACUAG |
| siRNA 4506 | 4506 | UGUAAAUUAAGGAUUACUC | 10860 | GAGUAAUCCUUAAUUUACA |
| siRNA 4508 | 4508 | UAAAUUAAGGAUUACUCAA | 10862 | UUGAGUAAUCCUUAAUUUA |
| siRNA 4510 | 4510 | AAUUAAGGAUUACUCAAGC | 10864 | GCUUGAGUAAUCCUUAAUU |
| siRNA 4511 | 4511 | AUUAAGGAUUACUCAAGCU | 10865 | AGCUUGAGUAAUCCUUAAU |
| siRNA 4512 | 4512 | UUAAGGAUUACUCAAGCUC | 10866 | GAGCUUGAGUAAUCCUUAA |
| siRNA 4517 | 4517 | GAUUACUCAAGCUCACCAU | 10871 | AUGGUGAGCUUGAGUAAUC |
| siRNA 4518 | 4518 | AUUACUCAAGCUCACCAUU | 10872 | AAUGGUGAGCUUGAGUAAU |
| siRNA 4519 | 4519 | UUACUCAAGCUCACCAUUA | 10873 | UAAUGGUGAGCUUGAGUAA |
| siRNA 4520 | 4520 | UACUCAAGCUCACCAUUAU | 10874 | AUAAUGGUGAGCUUGAGUA |
| siRNA 4521 | 4521 | ACUCAAGCUCACCAUUAUU | 10875 | AAUAAUGGUGAGCUUGAGU |
| siRNA 4525 | 4525 | AAGCUCACCAUUAUUCAAG | 10879 | CUUGAAUAAUGGUGAGCUU |
| siRNA 4526 | 4526 | AGCUCACCAUUAUUCAAGA | 10880 | UCUUGAAUAAUGGUGAGCU |
| siRNA 4529 | 4529 | UCACCAUUAUUCAAGAUUG | 10883 | CAAUCUUGAAUAAUGGUGA |
| siRNA 4531 | 4531 | ACCAUUAUUCAAGAUUGGG | 10885 | CCCAAUCUUGAAUAAUGGU |
| siRNA 4532 | 4532 | CCAUUAUUCAAGAUUGGGA | 10886 | UCCCAAUCUUGAAUAAUGG |
| siRNA 4535 | 4535 | UUAUUCAAGAUUGGGACUC | 10889 | GAGUCCCAAUCUUGAAUAA |
| siRNA 4536 | 4536 | UAUUCAAGAUUGGGACUCG | 10890 | CGAGUCCCAAUCUUGAAUA |
| siRNA 4537 | 4537 | AUUCAAGAUUGGGACUCGC | 10891 | GCGAGUCCCAAUCUUGAAU |
| siRNA 4538 | 4538 | UUCAAGAUUGGGACUCGCU | 10892 | AGCGAGUCCCAAUCUUGAA |
| siRNA 4540 | 4540 | CAAGAUUGGGACUCGCUUC | 10894 | GAAGCGAGUCCCAAUCUUG |
| siRNA 4541 | 4541 | AAGAUUGGGACUCGCUUCC | 10895 | GGAAGCGAGUCCCAAUCUU |
| siRNA 4543 | 4543 | GAUUGGGACUCGCUUCCCA | 10897 | UGGGAAGCGAGUCCCAAUC |
| siRNA 4548 | 4548 | GGACUCGCUUCCCAGUCGA | 10902 | UCGACUGGGAAGCGAGUCC |
| siRNA 4549 | 4549 | GACUCGCUUCCCAGUCGAC | 10903 | GUCGACUGGGAAGCGAGUC |
| siRNA 4551 | 4551 | CUCGCUUCCCAGUCGACAC | 10905 | GUGUCGACUGGGAAGCGAG |
| siRNA 4552 | 4552 | UCGCUUCCCAGUCGACACU | 10906 | AGUGUCGACUGGGAAGCGA |
| siRNA 4555 | 4555 | CUUCCCAGUCGACACUCUG | 10909 | CAGAGUGUCGACUGGGAAG |
| siRNA 4564 | 4564 | CGACACUCUGCCCUGCCUG | 10918 | CAGGCAGGGCAGAGUGUCG |
| siRNA 4577 | 4577 | UGCCUGUCAUUGCUGCAAA | 10931 | UUUGCAGCAAUGACAGGCA |
| siRNA 4600 | 4600 | UGCUGCUUUGCCAACCUAA | 10954 | UUAGGUUGGCAAAGCAGCA |
| siRNA 4602 | 4602 | CUGCUUUGCCAACCUAAGC | 10956 | GCUUAGGUUGGCAAAGCAG |
| siRNA 4603 | 4603 | UGCUUUGCCAACCUAAGCA | 10957 | UGCUUAGGUUGGCAAAGCA |
| siRNA 4604 | 4604 | GCUUUGCCAACCUAAGCAA | 10958 | UUGCUUAGGUUGGCAAAGC |
| siRNA 4615 | 4615 | CUAAGCAAAGAAAAUACGG | 10969 | CCGUAUUUUCUUUGCUUAG |
| siRNA 4621 | 4621 | AAAGAAAAUACGGCUUCUC | 10975 | GAGAAGCCGUAUUUUCUUU |
| siRNA 4623 | 4623 | AGAAAAUACGGCUUCUCUU | 10977 | AAGAGAAGCCGUAUUUUCU |
| siRNA 4663 | 4663 | GGUUUGUUUUCUAGAAGUA | 11017 | UACUUCUAGAAAACAAACC |
| siRNA 4665 | 4665 | UUUGUUUUCUAGAAGUACG | 11019 | CGUACUUCUAGAAAACAAA |
| siRNA 4666 | 4666 | UUGUUUUCUAGAAGUACGU | 11020 | ACGUACUUCUAGAAAACAA |
| siRNA 4667 | 4667 | UGUUUUCUAGAAGUACGUU | 11021 | AACGUACUUCUAGAAAACA |
| siRNA 4669 | 4669 | UUUUCUAGAAGUACGUUCA | 11023 | UGAACGUACUUCUAGAAAA |
| siRNA 4670 | 4670 | UUUCUAGAAGUACGUUCAG | 11024 | CUGAACGUACUUCUAGAAA |
| siRNA 4671 | 4671 | UUCUAGAAGUACGUUCAGA | 11025 | UCUGAACGUACUUCUAGAA |
| siRNA 4672 | 4672 | UCUAGAAGUACGUUCAGAU | 11026 | AUCUGAACGUACUUCUAGA |
| siRNA 4673 | 4673 | CUAGAAGUACGUUCAGAUG | 11027 | CAUCUGAACGUACUUCUAG |
| siRNA 4675 | 4675 | AGAAGUACGUUCAGAUGCU | 11029 | AGCAUCUGAACGUACUUCU |
| siRNA 4681 | 4681 | ACGUUCAGAUGCUUUGGGG | 11035 | CCCCAAAGCAUCUGAACGU |
| siRNA 4687 | 4687 | AGAUGCUUUGGGGAAUGCA | 11041 | UGCAUUCCCCAAAGCAUCU |
| siRNA 4694 | 4694 | UUGGGGAAUGCAAUGUAUG | 11048 | CAUACAUUGCAUUCCCCAA |
| siRNA 4695 | 4695 | UGGGGAAUGCAAUGUAUGA | 11049 | UCAUACAUUGCAUUCCCCA |
| siRNA 4708 | 4708 | GUAUGAUUUGCUAGCUCUC | 11062 | GAGAGCUAGCAAAUCAUAC |
| siRNA 4709 | 4709 | UAUGAUUUGCUAGCUCUCU | 11063 | AGAGAGCUAGCAAAUCAUA |
| siRNA 4710 | 4710 | AUGAUUUGCUAGCUCUCUC | 11064 | GAGAGAGCUAGCAAAUCAU |
| siRNA 4713 | 4713 | AUUUGCUAGCUCUCUCACC | 11067 | GGUGAGAGAGCUAGCAAAU |
| siRNA 4718 | 4718 | CUAGCUCUCUCACCACUUA | 11072 | UAAGUGGUGAGAGAGCUAG |
| siRNA 4723 | 4723 | UCUCUCACCACUUAACUCA | 11077 | UGAGUUAAGUGGUGAGAGA |
| siRNA 4725 | 4725 | UCUCACCACUUAACUCACU | 11079 | AGUGAGUUAAGUGGUGAGA |
| siRNA 4727 | 4727 | UCACCACUUAACUCACUGU | 11081 | ACAGUGAGUUAAGUGGUGA |
| siRNA 4732 | 4732 | ACUUAACUCACUGUGAGGA | 11086 | UCCUCACAGUGAGUUAAGU |
| siRNA 4734 | 4734 | UUAACUCACUGUGAGGAUA | 11088 | UAUCCUCACAGUGAGUUAA |
| siRNA 4735 | 4735 | UAACUCACUGUGAGGAUAA | 11089 | UUAUCCUCACAGUGAGUUA |
| siRNA 4737 | 4737 | ACUCACUGUGAGGAUAAAU | 11091 | AUUUAUCCUCACAGUGAGU |
| siRNA 4768 | 4768 | UGUAAUUAACUGGUGCUUU | 11122 | AAAGCACCAGUUAAUUACA |
| siRNA 4771 | 4771 | AAUUAACUGGUGCUUUGAA | 11125 | UUCAAAGCACCAGUUAAUU |
| siRNA 4772 | 4772 | AUUAACUGGUGCUUUGAAA | 11126 | UUUCAAAGCACCAGUUAAU |
| siRNA 4804 | 4804 | GAGAAAAAUCUCAACCAAA | 11158 | UUUGGUUGAGAUUUUUCUC |
| siRNA 4805 | 4805 | AGAAAAAUCUCAACCAAAG | 11159 | CUUUGGUUGAGAUUUUUCU |
| siRNA 4812 | 4812 | UCUCAACCAAAGUUAUGCU | 11166 | AGCAUAACUUUGGUUGAGA |
| siRNA 4814 | 4814 | UCAACCAAAGUUAUGCUCA | 11168 | UGAGCAUAACUUUGGUUGA |
| siRNA 4821 | 4821 | AAGUUAUGCUCAUCCAGAC | 11175 | GUCUGGAUGAGCAUAACUU |
| siRNA 4823 | 4823 | GUUAUGCUCAUCCAGACAA | 11177 | UUGUCUGGAUGAGCAUAAC |
| siRNA 4825 | 4825 | UAUGCUCAUCCAGACAAGC | 11179 | GCUUGUCUGGAUGAGCAUA |
| siRNA 4830 | 4830 | UCAUCCAGACAAGCUGACC | 11184 | GGUCAGCUUGUCUGGAUGA |
| siRNA 4838 | 4838 | ACAAGCUGACCUUUGAGUU | 11192 | AACUCAAAGGUCAGCUUGU |
| siRNA 4844 | 4844 | UGACCUUUGAGUUAAUUUC | 11198 | GAAAUUAACUCAAAGGUCA |
| siRNA 4856 | 4856 | UAAUUUCAGCACAACUCAU | 11210 | AUGAGUUGUGCUGAAAUUA |
| siRNA 4873 | 4873 | AUUCUUCAGUGCCUCAUGA | 11227 | UCAUGAGGCACUGAAGAAU |
| siRNA 4917 | 4917 | AAGCAUCUUCACAAUGAAG | 11271 | CUUCAUUGUGAAGAUGCUU |
| siRNA 4918 | 4918 | AGCAUCUUCACAAUGAAGC | 11272 | GCUUCAUUGUGAAGAUGCU |
| siRNA 4919 | 4919 | GCAUCUUCACAAUGAAGCU | 11273 | AGCUUCAUUGUGAAGAUGC |
| siRNA 4929 | 4929 | AAUGAAGCUUCCAGAUAGC | 11283 | GCUAUCUGGAAGCUUCAUU |
| siRNA 4931 | 4931 | UGAAGCUUCCAGAUAGCAC | 11285 | GUGCUAUCUGGAAGCUUCA |
| siRNA 4932 | 4932 | GAAGCUUCCAGAUAGCACC | 11286 | GGUGCUAUCUGGAAGCUUC |
| siRNA 4934 | 4934 | AGCUUCCAGAUAGCACCGU | 11288 | ACGGUGCUAUCUGGAAGCU |
| siRNA 4936 | 4936 | CUUCCAGAUAGCACCGUUU | 11290 | AAACGGUGCUAUCUGGAAG |
| siRNA 4937 | 4937 | UUCCAGAUAGCACCGUUUU | 11291 | AAAACGGUGCUAUCUGGAA |
| siRNA 4942 | 4942 | GAUAGCACCGUUUUGCUAA | 11296 | UUAGCAAAACGGUGCUAUC |
| siRNA 4943 | 4943 | AUAGCACCGUUUUGCUAAA | 11297 | UUUAGCAAAACGGUGCUAU |
| siRNA 4947 | 4947 | CACCGUUUUGCUAAAAGAU | 11301 | AUCUUUUAGCAAAACGGUG |
| siRNA 4948 | 4948 | ACCGUUUUGCUAAAAGAUA | 11302 | UAUCUUUUAGCAAAACGGU |
| siRNA 4949 | 4949 | CCGUUUUGCUAAAAGAUAC | 11303 | GUAUCUUUUAGCAAAACGG |
| siRNA 4973 | 4973 | CAUUGUUUUCCAACAGUGA | 11327 | UCACUGUUGGAAAACAAUG |
| siRNA 4993 | 4993 | GGCUUCCACAUAAGGUUAA | 11347 | UUAACCUUAUGUGGAAGCC |
| siRNA 4994 | 4994 | GCUUCCACAUAAGGUUAAA | 11348 | UUUAACCUUAUGUGGAAGC |
| siRNA 4996 | 4996 | UUCCACAUAAGGUUAAACA | 11350 | UGUUUAACCUUAUGUGGAA |
| siRNA 4997 | 4997 | UCCACAUAAGGUUAAACAA | 11351 | UUGUUUAACCUUAUGUGGA |
| siRNA 5002 | 5002 | AUAAGGUUAAACAAACUAG | 11356 | CUAGUUUGUUUAACCUUAU |
| siRNA 5003 | 5003 | UAAGGUUAAACAAACUAGG | 11357 | CCUAGUUUGUUUAACCUUA |
| siRNA 5005 | 5005 | AGGUUAAACAAACUAGGUG | 11359 | CACCUAGUUUGUUUAACCU |
| siRNA 5009 | 5009 | UAAACAAACUAGGUGCUUG | 11363 | CAAGCACCUAGUUUGUUUA |
| siRNA 5011 | 5011 | AACAAACUAGGUGCUUGUA | 11365 | UACAAGCACCUAGUUUGUU |
| siRNA 5012 | 5012 | ACAAACUAGGUGCUUGUAA | 11366 | UUACAAGCACCUAGUUUGU |
| siRNA 5013 | 5013 | CAAACUAGGUGCUUGUAAA | 11367 | UUUACAAGCACCUAGUUUG |
| siRNA 5016 | 5016 | ACUAGGUGCUUGUAAAUAA | 11370 | UUAUUUACAAGCACCUAGU |
| siRNA 5031 | 5031 | AUAAUUUAUUACAGUUUAC | 11385 | GUAAACUGUAAUAAAUUAU |
| siRNA 5036 | 5036 | UUAUUACAGUUUACUCUAU | 11390 | AUAGAGUAAACUGUAAUAA |
| siRNA 5037 | 5037 | UAUUACAGUUUACUCUAUC | 11391 | GAUAGAGUAAACUGUAAUA |
| siRNA 5039 | 5039 | UUACAGUUUACUCUAUCGC | 11393 | GCGAUAGAGUAAACUGUAA |
| siRNA 5040 | 5040 | UACAGUUUACUCUAUCGCA | 11394 | UGCGAUAGAGUAAACUGUA |
| siRNA 5042 | 5042 | CAGUUUACUCUAUCGCAUU | 11396 | AAUGCGAUAGAGUAAACUG |
| siRNA 5043 | 5043 | AGUUUACUCUAUCGCAUUU | 11397 | AAAUGCGAUAGAGUAAACU |
| siRNA 5044 | 5044 | GUUUACUCUAUCGCAUUUC | 11398 | GAAAUGCGAUAGAGUAAAC |
| siRNA 5046 | 5046 | UUACUCUAUCGCAUUUCUG | 11400 | CAGAAAUGCGAUAGAGUAA |
| siRNA 5047 | 5047 | UACUCUAUCGCAUUUCUGU | 11401 | ACAGAAAUGCGAUAGAGUA |
| siRNA 5052 | 5052 | UAUCGCAUUUCUGUAACAU | 11406 | AUGUUACAGAAAUGCGAUA |
| siRNA 5053 | 5053 | AUCGCAUUUCUGUAACAUG | 11407 | CAUGUUACAGAAAUGCGAU |
| siRNA 5067 | 5067 | ACAUGAAAUGCAUGCCCUU | 11421 | AAGGGCAUGCAUUUCAUGU |
| siRNA 5082 | 5082 | CCUUCUUCAGGGGAAGACU | 11436 | AGUCUUCCCCUGAAGAAGG |
| siRNA 5093 | 5093 | GGAAGACUGUGGUCAAGUU | 11447 | AACUUGACCACAGUCUUCC |
| siRNA 5101 | 5101 | GUGGUCAAGUUAAAAAAAA | 11455 | UUUUUUUUAACUUGACCAC |
| siRNA 5125 | 5125 | AAUAUUAAACAACAUGAAA | 11479 | UUUCAUGUUGUUUAAUAUU |
| siRNA 5156 | 5156 | UUUGAAAAUGAGAAUGUCC | 11510 | GGACAUUCUCAUUUUCAAA |
| siRNA 5162 | 5162 | AAUGAGAAUGUCCUAAGUG | 11516 | CACUUAGGACAUUCUCAUU |
| siRNA 5168 | 5168 | AAUGUCCUAAGUGAUUCAG | 11522 | CUGAAUCACUUAGGACAUU |
| siRNA 5170 | 5170 | UGUCCUAAGUGAUUCAGAA | 11524 | UUCUGAAUCACUUAGGACA |
| siRNA 5192 | 5192 | AGGAGGGAAGUUGUGCACU | 11546 | AGUGCACAACUUCCCUCCU |
| siRNA 5194 | 5194 | GAGGGAAGUUGUGCACUCU | 11548 | AGAGUGCACAACUUCCCUC |
| siRNA 5202 | 5202 | UUGUGCACUCUGAAAAUGC | 11556 | GCAUUUUCAGAGUGCACAA |
| siRNA 5205 | 5205 | UGCACUCUGAAAAUGCAUG | 11559 | CAUGCAUUUUCAGAGUGCA |
| siRNA 5218 | 5218 | UGCAUGAAAAACAAAGGCA | 11572 | UGCCUUUGUUUUUCAUGCA |
| siRNA 5233 | 5233 | GGCAAAAACUAGUGGGAAA | 11587 | UUUCCCACUAGUUUUUGCC |
| siRNA 5240 | 5240 | ACUAGUGGGAAAUGUGUAG | 11594 | CUACACAUUUCCCACUAGU |
| siRNA 5242 | 5242 | UAGUGGGAAAUGUGUAGAA | 11596 | UUCUACACAUUUCCCACUA |
| siRNA 5244 | 5244 | GUGGGAAAUGUGUAGAACU | 11598 | AGUUCUACACAUUUCCCAC |
| siRNA 5246 | 5246 | GGGAAAUGUGUAGAACUGU | 11600 | ACAGUUCUACACAUUUCCC |
| siRNA 5251 | 5251 | AUGUGUAGAACUGUUAACU | 11605 | AGUUAACAGUUCUACACAU |
| siRNA 5279 | 5279 | UCGAGUCUUCCUUCUGGAA | 11633 | UUCCAGAAGGAAGACUCGA |
| siRNA 5301 | 5301 | GUUAAAUUUCACAAAGUCA | 11655 | UGACUUUGUGAAAUUUAAC |
| siRNA 5312 | 5312 | CAAAGUCAUGAGGGUAAAU | 11666 | AUUUACCCUCAUGACUUUG |
| siRNA 5314 | 5314 | AAGUCAUGAGGGUAAAUGG | 11668 | CCAUUUACCCUCAUGACUU |
| siRNA 5318 | 5318 | CAUGAGGGUAAAUGGAGAA | 11672 | UUCUCCAUUUACCCUCAUG |
| siRNA 5340 | 5340 | AUUUCUGGGAUUACAAUGA | 11694 | UCAUUGUAAUCCCAGAAAU |
| siRNA 5349 | 5349 | AUUACAAUGAAUGUAAGCC | 11703 | GGCUUACAUUCAUUGUAAU |
| siRNA 5353 | 5353 | CAAUGAAUGUAAGCCCAAA | 11707 | UUUGGGCUUACAUUCAUUG |
| siRNA 5359 | 5359 | AUGUAAGCCCAAAUUGUGG | 11713 | CCACAAUUUGGGCUUACAU |
| siRNA 5360 | 5360 | UGUAAGCCCAAAUUGUGGA | 11714 | UCCACAAUUUGGGCUUACA |
| siRNA 5364 | 5364 | AGCCCAAAUUGUGGAAUUG | 11718 | CAAUUCCACAAUUUGGGCU |
| siRNA 5385 | 5385 | AGUAACCUGGAUGGGGAAA | 11739 | UUUCCCCAUCCAGGUUACU |
| siRNA 5395 | 5395 | AUGGGGAAAAGCAUUUCCC | 11749 | GGGAAAUGCUUUUCCCCAU |
| siRNA 5403 | 5403 | AAGCAUUUCCCAUAGCACU | 11757 | AGUGCUAUGGGAAAUGCUU |
| siRNA 5411 | 5411 | CCCAUAGCACUCCAUGUAA | 11765 | UUACAUGGAGUGCUAUGGG |
| siRNA 5413 | 5413 | CAUAGCACUCCAUGUAAUA | 11767 | UAUUACAUGGAGUGCUAUG |
| siRNA 5417 | 5417 | GCACUCCAUGUAAUAUGAG | 11771 | CUCAUAUUACAUGGAGUGC |
| siRNA 5421 | 5421 | UCCAUGUAAUAUGAGUGCU | 11775 | AGCACUCAUAUUACAUGGA |
| siRNA 5422 | 5422 | CCAUGUAAUAUGAGUGCUC | 11776 | GAGCACUCAUAUUACAUGG |
| siRNA 5430 | 5430 | UAUGAGUGCUCUGUGAGAU | 11784 | AUCUCACAGAGCACUCAUA |
| siRNA 5432 | 5432 | UGAGUGCUCUGUGAGAUGU | 11786 | ACAUCUCACAGAGCACUCA |
| siRNA 5441 | 5441 | UGUGAGAUGUUCAUCAGUG | 11795 | CACUGAUGAACAUCUCACA |
| siRNA 5462 | 5462 | UUAUAGAAAUGGUGUUGCU | 11816 | AGCAACACCAUUUCUAUAA |
| siRNA 5463 | 5463 | UAUAGAAAUGGUGUUGCUG | 11817 | CAGCAACACCAUUUCUAUA |
| siRNA 5477 | 5477 | UGCUGGGAAACCAAGUUUG | 11831 | CAAACUUGGUUUCCCAGCA |
| siRNA 5485 | 5485 | AACCAAGUUUGCACCUGGA | 11839 | UCCAGGUGCAAACUUGGUU |
| siRNA 5486 | 5486 | ACCAAGUUUGCACCUGGAA | 11840 | UUCCAGGUGCAAACUUGGU |
| siRNA 5487 | 5487 | CCAAGUUUGCACCUGGAAA | 11841 | UUUCCAGGUGCAAACUUGG |
| siRNA 5497 | 5497 | ACCUGGAAACUUACAAUGC | 11851 | GCAUUGUAAGUUUCCAGGU |
| siRNA 5498 | 5498 | CCUGGAAACUUACAAUGCA | 11852 | UGCAUUGUAAGUUUCCAGG |
| siRNA 5500 | 5500 | UGGAAACUUACAAUGCACU | 11854 | AGUGCAUUGUAAGUUUCCA |
| siRNA 5505 | 5505 | ACUUACAAUGCACUUUAGC | 11859 | GCUAAAGUGCAUUGUAAGU |
| siRNA 5507 | 5507 | UUACAAUGCACUUUAGCGC | 11861 | GCGCUAAAGUGCAUUGUAA |
| siRNA 5508 | 5508 | UACAAUGCACUUUAGCGCA | 11862 | UGCGCUAAAGUGCAUUGUA |
| siRNA 5509 | 5509 | ACAAUGCACUUUAGCGCAG | 11863 | CUGCGCUAAAGUGCAUUGU |
| siRNA 5510 | 5510 | CAAUGCACUUUAGCGCAGU | 11864 | ACUGCGCUAAAGUGCAUUG |
| siRNA 5512 | 5512 | AUGCACUUUAGCGCAGUAA | 11866 | UUACUGCGCUAAAGUGCAU |
| siRNA 5513 | 5513 | UGCACUUUAGCGCAGUAAG | 11867 | CUUACUGCGCUAAAGUGCA |
| siRNA 5514 | 5514 | GCACUUUAGCGCAGUAAGG | 11868 | CCUUACUGCGCUAAAGUGC |
| siRNA 5515 | 5515 | CACUUUAGCGCAGUAAGGG | 11869 | CCCUUACUGCGCUAAAGUG |
| siRNA 5516 | 5516 | ACUUUAGCGCAGUAAGGGC | 11870 | GCCCUUACUGCGCUAAAGU |
| siRNA 5517 | 5517 | CUUUAGCGCAGUAAGGGCU | 11871 | AGCCCUUACUGCGCUAAAG |
| siRNA 5521 | 5521 | AGCGCAGUAAGGGCUUGGC | 11875 | GCCAAGCCCUUACUGCGCU |
| siRNA 5522 | 5522 | GCGCAGUAAGGGCUUGGCA | 11876 | UGCCAAGCCCUUACUGCGC |
| siRNA 5523 | 5523 | CGCAGUAAGGGCUUGGCAU | 11877 | AUGCCAAGCCCUUACUGCG |
| siRNA 5526 | 5526 | AGUAAGGGCUUGGCAUCCG | 11880 | CGGAUGCCAAGCCCUUACU |
| siRNA 5527 | 5527 | GUAAGGGCUUGGCAUCCGG | 11881 | CCGGAUGCCAAGCCCUUAC |
| siRNA 5528 | 5528 | UAAGGGCUUGGCAUCCGGU | 11882 | ACCGGAUGCCAAGCCCUUA |
| siRNA 5529 | 5529 | AAGGGCUUGGCAUCCGGUA | 11883 | UACCGGAUGCCAAGCCCUU |
| siRNA 5531 | 5531 | GGGCUUGGCAUCCGGUAGU | 11885 | ACUACCGGAUGCCAAGCCC |
| siRNA 5532 | 5532 | GGCUUGGCAUCCGGUAGUG | 11886 | CACUACCGGAUGCCAAGCC |
| siRNA 5533 | 5533 | GCUUGGCAUCCGGUAGUGA | 11887 | UCACUACCGGAUGCCAAGC |
| siRNA 5535 | 5535 | UUGGCAUCCGGUAGUGAAA | 11889 | UUUCACUACOGGAUGCCAA |
| siRNA 5536 | 5536 | UGGCAUCCGGUAGUGAAAA | 11890 | UUUUCACUACCGGAUGCCA |
| siRNA 5539 | 5539 | CAUCCGGUAGUGAAAAACU | 11893 | AGUUUUUCACUACCGGAUG |
| siRNA 5540 | 5540 | AUCCGGUAGUGAAAAACUG | 11894 | CAGUUUUUCACUACCGGAU |
| siRNA 5541 | 5541 | UCCGGUAGUGAAAAACUGU | 11895 | ACAGUUUUUCACUACCGGA |
| siRNA 5542 | 5542 | CCGGUAGUGAAAAACUGUC | 11896 | GACAGUUUUUCACUACCGG |
| siRNA 5545 | 5545 | GUAGUGAAAAACUGUCUAA | 11899 | UUAGACAGUUUUUCACUAC |
| siRNA 5547 | 5547 | AGUGAAAAACUGUCUAACC | 11901 | GGUUAGACAGUUUUUCACU |
| siRNA 5548 | 5548 | GUGAAAAACUGUCUAACCC | 11902 | GGGUUAGACAGUUUUUCAC |
| siRNA 5556 | 5556 | CUGUCUAACCCAGCAUUGC | 11910 | GCAAUGCUGGGUUAGACAG |
| siRNA 5557 | 5557 | UGUCUAACCCAGCAUUGCC | 11911 | GGCAAUGCUGGGUUAGACA |
| siRNA 5560 | 5560 | CUAACCCAGCAUUGCCCAA | 11914 | UUGGGCAAUGCUGGGUUAG |
| siRNA 5565 | 5565 | CCAGCAUUGCCCAAACUAU | 11919 | AUAGUUUGGGCAAUGCUGG |
| siRNA 5567 | 5567 | AGCAUUGCCCAAACUAUUU | 11921 | AAAUAGUUUGGGCAAUGCU |
| siRNA 5568 | 5568 | GCAUUGCCCAAACUAUUUU | 11922 | AAAAUAGUUUGGGCAAUGC |
| siRNA 5572 | 5572 | UGCCCAAACUAUUUUGACA | 11926 | UGUCAAAAUAGUUUGGGCA |
| siRNA 5573 | 5573 | GCCCAAACUAUUUUGACAC | 11927 | GUGUCAAAAUAGUUUGGGC |
| siRNA 5574 | 5574 | CCCAAACUAUUUUGACACC | 11928 | GGUGUCAAAAUAGUUUGGG |
| siRNA 5575 | 5575 | CCAAACUAUUUUGACACCA | 11929 | UGGUGUCAAAAUAGUUUGG |
| siRNA 5578 | 5578 | AACUAUUUUGACACCAGGA | 11932 | UCCUGGUGUCAAAAUAGUU |
| siRNA 5580 | 5580 | CUAUUUUGACACCAGGACC | 11934 | GGUCCUGGUGUCAAAAUAG |
| siRNA 5581 | 5581 | UAUUUUGACACCAGGACCU | 11935 | AGGUCCUGGUGUCAAAAUA |
| siRNA 5601 | 5601 | UUUCUCCUUUGGGAUACUU | 11955 | AAGUAUCCCAAAGGAGAAA |
| siRNA 5603 | 5603 | UCUCCUUUGGGAUACUUAU | 11957 | AUAAGUAUCCCAAAGGAGA |
| siRNA 5604 | 5604 | CUCCUUUGGGAUACUUAUG | 11958 | CAUAAGUAUCCCAAAGGAG |
| siRNA 5605 | 5605 | UCCUUUGGGAUACUUAUGA | 11959 | UCAUAAGUAUCCCAAAGGA |
| siRNA 5608 | 5608 | UUUGGGAUACUUAUGAACC | 11962 | GGUUCAUAAGUAUCCCAAA |
| siRNA 5610 | 5610 | UGGGAUACUUAUGAACCUC | 11964 | GAGGUUCAUAAGUAUCCCA |
| siRNA 5614 | 5614 | AUACUUAUGAACCUCUCAC | 11968 | GUGAGAGGUUCAUAAGUAU |
| siRNA 5617 | 5617 | CUUAUGAACCUCUCACUAA | 11971 | UUAGUGAGAGGUUCAUAAG |
| siRNA 5618 | 5618 | UUAUGAACCUCUCACUAAU | 11972 | AUUAGUGAGAGGUUCAUAA |
| siRNA 5622 | 5622 | GAACCUCUCACUAAUGUCC | 11976 | GGACAUUAGUGAGAGGUUC |
| siRNA 5623 | 5623 | AACCUCUCACUAAUGUCCU | 11977 | AGGACAUUAGUGAGAGGUU |
| siRNA 5625 | 5625 | CCUCUCACUAAUGUCCUGU | 11979 | ACAGGACAUUAGUGAGAGG |
| siRNA 5626 | 5626 | CUCUCACUAAUGUCCUGUG | 11980 | CACAGGACAUUAGUGAGAG |
| siRNA 5630 | 5630 | CACUAAUGUCCUGUGGAGA | 11984 | UCUCCACAGGACAUUAGUG |
| siRNA 5631 | 5631 | ACUAAUGUCCUGUGGAGAA | 11985 | UUCUCCACAGGACAUUAGU |
| siRNA 5633 | 5633 | UAAUGUCCUGUGGAGAACA | 11987 | UGUUCUCCACAGGACAUUA |
| siRNA 5643 | 5643 | UGGAGAACAUUUUGGGAAA | 11997 | UUUCCCAAAAUGUUCUCCA |
| siRNA 5650 | 5650 | CAUUUUGGGAAACACUAUG | 12004 | CAUAGUGUUUCCCAAAAUG |
| siRNA 5651 | 5651 | AUUUUGGGAAACACUAUGU | 12005 | ACAUAGUGUUUCCCAAAAU |
| siRNA 5660 | 5660 | AACACUAUGUUAGAUAGUU | 12014 | AACUAUCUAACAUAGUGUU |
| siRNA 5661 | 5661 | ACACUAUGUUAGAUAGUUC | 12015 | GAACUAUCUAACAUAGUGU |
| siRNA 5662 | 5662 | CACUAUGUUAGAUAGUUCU | 12016 | AGAACUAUCUAACAUAGUG |
| siRNA 5663 | 5663 | ACUAUGUUAGAUAGUUCUU | 12017 | AAGAACUAUCUAACAUAGU |
| siRNA 5664 | 5664 | CUAUGUUAGAUAGUUCUUU | 12018 | AAAGAACUAUCUAACAUAG |
| siRNA 5673 | 5673 | AUAGUUCUUUAAGGAGACA | 12027 | UGUCUCCUUAAAGAACUAU |
| siRNA 5679 | 5679 | CUUUAAGGAGACAAAACGG | 12033 | CCGUUUUGUCUCCUUAAAG |
| siRNA 5680 | 5680 | UUUAAGGAGACAAAACGGU | 12034 | ACCGUUUUGUCUCCUUAAA |
| siRNA 5681 | 5681 | UUAAGGAGACAAAACGGUA | 12035 | UACCGUUUUGUCUCCUUAA |
| siRNA 5682 | 5682 | UAAGGAGACAAAACGGUAA | 12036 | UUACCGUUUUGUCUCCUUA |
| siRNA 5683 | 5683 | AAGGAGACAAAACGGUAAU | 12037 | AUUACCGUUUUGUCUCCUU |
| siRNA 5687 | 5687 | AGACAAAACGGUAAUGAAC | 12041 | GUUCAUUACCGUUUUGUCU |
| siRNA 5688 | 5688 | GACAAAACGGUAAUGAACA | 12042 | UGUUCAUUACCGUUUUGUC |
| siRNA 5689 | 5689 | ACAAAACGGUAAUGAACAG | 12043 | CUGUUCAUUACCGUUUUGU |
| siRNA 5693 | 5693 | AACGGUAAUGAACAGAUAG | 12047 | CUAUCUGUUCAUUACCGUU |
| siRNA 5694 | 5694 | ACGGUAAUGAACAGAUAGC | 12048 | GCUAUCUGUUCAUUACCGU |
| siRNA 5696 | 5696 | GGUAAUGAACAGAUAGCAC | 12050 | GUGCUAUCUGUUCAUUACC |
| siRNA 5701 | 5701 | UGAACAGAUAGCACUGGGG | 12055 | CCCCAGUGCUAUCUGUUCA |
| siRNA 5712 | 5712 | CACUGGGGCAGAAUAUGCA | 12066 | UGCAUAUUCUGCCCCAGUG |
| siRNA 5714 | 5714 | CUGGGGCAGAAUAUGCAUG | 12068 | CAUGCAUAUUCUGCCCCAG |
| siRNA 5716 | 5716 | GGGGCAGAAUAUGCAUGCA | 12070 | UGCAUGCAUAUUCUGCCCC |
| siRNA 5718 | 5718 | GGCAGAAUAUGCAUGCAUU | 12072 | AAUGCAUGCAUAUUCUGCC |
| siRNA 5724 | 5724 | AUAUGCAUGCAUUUUGUAA | 12078 | UUACAAAAUGCAUGCAUAU |
| siRNA 5727 | 5727 | UGCAUGCAUUUUGUAACGU | 12081 | ACGUUACAAAAUGCAUGCA |
| siRNA 5728 | 5728 | GCAUGCAUUUUGUAACGUC | 12082 | GACGUUACAAAAUGCAUGC |
| siRNA 5733 | 5733 | CAUUUUGUAACGUCCAGUG | 12087 | CACUGGACGUUACAAAAUG |
| siRNA 5734 | 5734 | AUUUUGUAACGUCCAGUGU | 12088 | ACACUGGACGUUACAAAAU |
| siRNA 5739 | 5739 | GUAACGUCCAGUGUGGCGU | 12093 | ACGCCACACUGGACGUUAC |
| siRNA 5740 | 5740 | UAACGUCCAGUGUGGCGUU | 12094 | AACGCCACACUGGACGUUA |
| siRNA 5741 | 5741 | AACGUCCAGUGUGGCGUUG | 12095 | CAACGCCACACUGGACGUU |
| siRNA 5746 | 5746 | CCAGUGUGGCGUUGAAUAG | 12100 | CUAUUCAACGCCACACUGG |
| siRNA 5748 | 5748 | AGUGUGGCGUUGAAUAGAU | 12102 | AUCUAUUCAACGCCACACU |
| siRNA 5749 | 5749 | GUGUGGCGUUGAAUAGAUG | 12103 | CAUCUAUUCAACGCCACAC |
| siRNA 5751 | 5751 | GUGGCGUUGAAUAGAUGUG | 12105 | CACAUCUAUUCAACGCCAC |
| siRNA 5752 | 5752 | UGGCGUUGAAUAGAUGUGU | 12106 | ACACAUCUAUUCAACGCCA |
| siRNA 5753 | 5753 | GGCGUUGAAUAGAUGUGUA | 12107 | UACACAUCUAUUCAACGCC |
| siRNA 5754 | 5754 | GCGUUGAAUAGAUGUGUAU | 12108 | AUACACAUCUAUUCAACGC |
| siRNA 5763 | 5763 | AGAUGUGUAUUUCCUCCCC | 12117 | GGGGAGGAAAUACACAUCU |
| siRNA 5768 | 5768 | UGUAUUUCCUCCCCUGCAG | 12122 | CUGCAGGGGAGGAAAUACA |
| siRNA 5782 | 5782 | UGCAGAAAAUAAGCACAGA | 12136 | UCUGUGCUUAUUUUCUGCA |
| siRNA 5798 | 5798 | AGAAAAUUAUAAUGUAGGU | 12152 | ACCUACAUUAUAAUUUUCU |
| siRNA 5800 | 5800 | AAAAUUAUAAUGUAGGUGA | 12154 | UCACCUACAUUAUAAUUUU |
| siRNA 5801 | 5801 | AAAUUAUAAUGUAGGUGAU | 12155 | AUCACCUACAUUAUAAUUU |
| siRNA 5802 | 5802 | AAUUAUAAUGUAGGUGAUC | 12156 | GAUCACCUACAUUAUAAUU |
| siRNA 5803 | 5803 | AUUAUAAUGUAGGUGAUCG | 12157 | CGAUCACCUACAUUAUAAU |
| siRNA 5805 | 5805 | UAUAAUGUAGGUGAUCGGA | 12159 | UCCGAUCACCUACAUUAUA |
| siRNA 5807 | 5807 | UAAUGUAGGUGAUCGGAGC | 12161 | GCUCCGAUCACCUACAUUA |
| siRNA 5810 | 5810 | UGUAGGUGAUCGGAGCUCU | 12164 | AGAGCUCCGAUCACCUACA |
| siRNA 5811 | 5811 | GUAGGUGAUCGGAGCUCUU | 12165 | AAGAGCUCCGAUCACCUAC |
| siRNA 5812 | 5812 | UAGGUGAUCGGAGCUCUUU | 12166 | AAAGAGCUCCGAUCACCUA |
| siRNA 5813 | 5813 | AGGUGAUCGGAGCUCUUUC | 12167 | GAAAGAGCUCCGAUCACCU |
| siRNA 5817 | 5817 | GAUCGGAGCUCUUUCCUUU | 12171 | AAAGGAAAGAGCUCCGAUC |
| siRNA 5819 | 5819 | UCGGAGCUCUUUCCUUUGA | 12173 | UCAAAGGAAAGAGCUCCGA |
| siRNA 5820 | 5820 | CGGAGCUCUUUCCUUUGAU | 12174 | AUCAAAGGAAAGAGCUCCG |
| siRNA 5830 | 5830 | UCCUUUGAUAGAGAGAACA | 12184 | UGUUCUCUCUAUCAAAGGA |
| siRNA 5833 | 5833 | UUUGAUAGAGAGAACAGCC | 12187 | GGCUGUUCUCUCUAUCAAA |
| siRNA 5835 | 5835 | UGAUAGAGAGAACAGCCCC | 12189 | GGGGCUGUUCUCUCUAUCA |
| siRNA 5839 | 5839 | AGAGAGAACAGCCCCAAUG | 12193 | CAUUGGGGCUGUUCUCUCU |
| siRNA 5849 | 5849 | GCCCCAAUGAUCCUGGCUU | 12203 | AAGCCAGGAUCAUUGGGGC |
| siRNA 5865 | 5865 | CUUUUUCACUGAACGUAUC | 12219 | GAUACGUUCAGUGAAAAAG |
| siRNA 5866 | 5866 | UUUUUCACUGAACGUAUCA | 12220 | UGAUACGUUCAGUGAAAAA |
| siRNA 5867 | 5867 | UUUUCACUGAACGUAUCAG | 12221 | CUGAUACGUUCAGUGAAAA |
| siRNA 5868 | 5868 | UUUCACUGAACGUAUCAGA | 12222 | UCUGAUACGUUCAGUGAAA |
| siRNA 5872 | 5872 | ACUGAACGUAUCAGAAUAC | 12226 | GUAUUCUGAUACGUUCAGU |
| siRNA 5873 | 5873 | CUGAACGUAUCAGAAUACA | 12227 | UGUAUUCUGAUACGUUCAG |
| siRNA 5875 | 5875 | GAACGUAUCAGAAUACAUG | 12229 | CAUGUAUUCUGAUACGUUC |
| siRNA 5891 | 5891 | AUGGAUGAAUUGGGGUAAA | 12245 | UUUACCCCAAUUCAUCCAU |
| siRNA 5892 | 5892 | UGGAUGAAUUGGGGUAAAU | 12246 | AUUUACCCCAAUUCAUCCA |
| siRNA 5902 | 5902 | GGGGUAAAUAAGGUUUUAA | 12256 | UUAAAACCUUAUUUACCCC |
| siRNA 5917 | 5917 | UUAAUUCAGAUCUAGAAGA | 12271 | UCUUCUAGAUCUGAAUUAA |
| siRNA 5959 | 5959 | AUUUUUAUCCACAGAUAGU | 12313 | ACUAUCUGUGGAUAAAAAU |
| siRNA 5964 | 5964 | UAUCCACAGAUAGUUGUAG | 12318 | CUACAACUAUCUGUGGAUA |
| siRNA 5965 | 5965 | AUCCACAGAUAGUUGUAGU | 12319 | ACUACAACUAUCUGUGGAU |
| siRNA 5966 | 5966 | UCCACAGAUAGUUGUAGUG | 12320 | CACUACAACUAUCUGUGGA |
| siRNA 5967 | 5967 | CCACAGAUAGUUGUAGUGU | 12321 | ACACUACAACUAUCUGUGG |
| siRNA 5968 | 5968 | CACAGAUAGUUGUAGUGUU | 12322 | AACACUACAACUAUCUGUG |
| siRNA 5971 | 5971 | AGAUAGUUGUAGUGUUUAG | 12325 | CUAAACACUACAACUAUCU |
| siRNA 5972 | 5972 | GAUAGUUGUAGUGUUUAGA | 12326 | UCUAAACACUACAACUAUC |
| siRNA 5973 | 5973 | AUAGUUGUAGUGUUUAGAC | 12327 | GUCUAAACACUACAACUAU |
| siRNA 5974 | 5974 | UAGUUGUAGUGUUUAGACA | 12328 | UGUCUAAACACUACAACUA |
| siRNA 5977 | 5977 | UUGUAGUGUUUAGACAUGA | 12331 | UCAUGUCUAAACACUACAA |
| siRNA 5983 | 5983 | UGUUUAGACAUGACAGGAC | 12337 | GUCCUGUCAUGUCUAAACA |
| siRNA 5984 | 5984 | GUUUAGACAUGACAGGACC | 12338 | GGUCCUGUCAUGUCUAAAC |
| siRNA 5986 | 5986 | UUAGACAUGACAGGACCUA | 12340 | UAGGUCCUGUCAUGUCUAA |
| siRNA 5987 | 5987 | UAGACAUGACAGGACCUAU | 12341 | AUAGGUCCUGUCAUGUCUA |
| siRNA 5988 | 5988 | AGACAUGACAGGACCUAUC | 12342 | GAUAGGUCCUGUCAUGUCU |
| siRNA 5990 | 5990 | ACAUGACAGGACCUAUCGU | 12344 | ACGAUAGGUCCUGUCAUGU |
| siRNA 5992 | 5992 | AUGACAGGACCUAUCGUUG | 12346 | CAACGAUAGGUCCUGUCAU |
| siRNA 5993 | 5993 | UGACAGGACCUAUCGUUGA | 12347 | UCAACGAUAGGUCCUGUCA |
| siRNA 5994 | 5994 | GACAGGACCUAUCGUUGAG | 12348 | CUCAACGAUAGGUCCUGUC |
| siRNA 5995 | 5995 | ACAGGACCUAUCGUUGAGG | 12349 | CCUCAACGAUAGGUCCUGU |
| siRNA 5996 | 5996 | CAGGACCUAUCGUUGAGGU | 12350 | ACCUCAACGAUAGGUCCUG |
| siRNA 5997 | 5997 | AGGACCUAUCGUUGAGGUU | 12351 | AACCUCAACGAUAGGUCCU |
| siRNA 5998 | 5998 | GGACCUAUCGUUGAGGUUU | 12352 | AAACCUCAACGAUAGGUCC |
| siRNA 5999 | 5999 | GACCUAUCGUUGAGGUUUC | 12353 | GAAACCUCAACGAUAGGUC |
| siRNA 6001 | 6001 | CCUAUCGUUGAGGUUUCUA | 12355 | UAGAAACCUCAACGAUAGG |
| siRNA 6005 | 6005 | UCGUUGAGGUUUCUAAGAC | 12359 | GUCUUAGAAACCUCAACGA |
| siRNA 6007 | 6007 | GUUGAGGUUUCUAAGACUU | 12361 | AAGUCUUAGAAACCUCAAC |
| siRNA 6010 | 6010 | GAGGUUUCUAAGACUUACU | 12364 | AGUAAGUCUUAGAAACCUC |
| siRNA 6012 | 6012 | GGUUUCUAAGACUUACUAU | 12366 | AUAGUAAGUCUUAGAAACC |
| siRNA 6016 | 6016 | UCUAAGACUUACUAUGGGC | 12370 | GCCCAUAGUAAGUCUUAGA |
| siRNA 6018 | 6018 | UAAGACUUACUAUGGGCUG | 12372 | CAGCCCAUAGUAAGUCUUA |
| siRNA 6028 | 6028 | UAUGGGCUGUAAACCUGUU | 12382 | AACAGGUUUACAGCCCAUA |
| siRNA 6029 | 6029 | AUGGGCUGUAAACCUGUUU | 12383 | AAACAGGUUUACAGCCCAU |
| siRNA 6032 | 6032 | GGCUGUAAACCUGUUUUUU | 12386 | AAAAAACAGGUUUACAGCC |
| siRNA 6056 | 6056 | UAUUUUAGAAACCUGAGAC | 12410 | GUCUCAGGUUUCUAAAAUA |
| siRNA 6061 | 6061 | UAGAAACCUGAGACUUGCC | 12415 | GGCAAGUCUCAGGUUUCUA |
| siRNA 6063 | 6063 | GAAACCUGAGACUUGCCGU | 12417 | ACGGCAAGUCUCAGGUUUC |
| siRNA 6065 | 6065 | AACCUGAGACUUGCCGUCU | 12419 | AGACGGCAAGUCUCAGGUU |
| siRNA 6067 | 6067 | CCUGAGACUUGCCGUCUGG | 12421 | CCAGACGGCAAGUCUCAGG |
| siRNA 6070 | 6070 | GAGACUUGCCGUCUGGCAU | 12424 | AUGCCAGACGGCAAGUCUC |
| siRNA 6071 | 6071 | AGACUUGCCGUCUGGCAUU | 12425 | AAUGCCAGACGGCAAGUCU |
| siRNA 6078 | 6078 | CCGUCUGGCAUUUUAGUUU | 12432 | AAACUAAAAUGCCAGACGG |
| siRNA 6098 | 6098 | AUACAAACUAAUGAUUGCA | 12452 | UGCAAUCAUUAGUUUGUAU |
| siRNA 6113 | 6113 | UGCAUUUGAAAGAGAUUCU | 12467 | AGAAUCUCUUUCAAAUGCA |
| siRNA 6132 | 6132 | UGACCUUAUUUCUAAACGU | 12486 | ACGUUUAGAAAUAAGGUCA |
| siRNA 6133 | 6133 | GACCUUAUUUCUAAACGUC | 12487 | GACGUUUAGAAAUAAGGUC |
| siRNA 6134 | 6134 | ACCUUAUUUCUAAACGUCU | 12488 | AGACGUUUAGAAAUAAGGU |
| siRNA 6136 | 6136 | CUUAUUUCUAAACGUCUAG | 12490 | CUAGACGUUUAGAAAUAAG |
| siRNA 6137 | 6137 | UUAUUUCUAAACGUCUAGA | 12491 | UCUAGACGUUUAGAAAUAA |
| siRNA 6138 | 6138 | UAUUUCUAAACGUCUAGAG | 12492 | CUCUAGACGUUUAGAAAUA |
| siRNA 6139 | 6139 | AUUUCUAAACGUCUAGAGC | 12493 | GCUCUAGACGUUUAGAAAU |
| siRNA 6141 | 6141 | UUCUAAACGUCUAGAGCUC | 12495 | GAGCUCUAGACGUUUAGAA |
| siRNA 6142 | 6142 | UCUAAACGUCUAGAGCUCU | 12496 | AGAGCUCUAGACGUUUAGA |
| siRNA 6145 | 6145 | AAACGUCUAGAGCUCUGAA | 12499 | UUCAGAGCUCUAGACGUUU |
| siRNA 6146 | 6146 | AACGUCUAGAGCUCUGAAA | 12500 | UUUCAGAGCUCUAGACGUU |
| siRNA 6148 | 6148 | CGUCUAGAGCUCUGAAAUG | 12502 | CAUUUCAGAGCUCUAGACG |
| siRNA 6169 | 6169 | UUGAUGGAAGGUAUUAAAC | 12523 | GUUUAAUACCUUCCAUCAA |
| siRNA 6170 | 6170 | UGAUGGAAGGUAUUAAACU | 12524 | AGUUUAAUACCUUCCAUCA |
| siRNA 6177 | 6177 | AGGUAUUAAACUAUUUGCC | 12531 | GGCAAAUAGUUUAAUACCU |
| siRNA 6178 | 6178 | GGUAUUAAACUAUUUGCCU | 12532 | AGGCAAAUAGUUUAAUACC |
| siRNA 6179 | 6179 | GUAUUAAACUAUUUGCCUG | 12533 | CAGGCAAAUAGUUUAAUAC |
| siRNA 6184 | 6184 | AAACUAUUUGCCUGUUGUA | 12538 | UACAACAGGCAAAUAGUUU |
| siRNA 6189 | 6189 | AUUUGCCUGUUGUACAAAG | 12543 | CUUUGUACAACAGGCAAAU |
| siRNA 6192 | 6192 | UGCCUGUUGUACAAAGAAA | 12546 | UUUCUUUGUACAACAGGCA |
| siRNA 6204 | 6204 | AAAGAAAUGUUAAGACUCG | 12558 | CGAGUCUUAACAUUUCUUU |
| siRNA 6205 | 6205 | AAGAAAUGUUAAGACUCGU | 12559 | ACGAGUCUUAACAUUUCUU |
| siRNA 6209 | 6209 | AAUGUUAAGACUCGUGAAA | 12563 | UUUCACGAGUCUUAACAUU |
| siRNA 6211 | 6211 | UGUUAAGACUCGUGAAAAG | 12565 | CUUUUCACGAGUCUUAACA |
| siRNA 6212 | 6212 | GUUAAGACUCGUGAAAAGA | 12566 | UCUUUUCACGAGUCUUAAC |
| siRNA 6219 | 6219 | CUCGUGAAAAGAAUUACUA | 12573 | UAGUAAUUCUUUUCACGAG |
| siRNA 6220 | 6220 | UCGUGAAAAGAAUUACUAU | 12574 | AUAGUAAUUCUUUUCACGA |
| siRNA 6228 | 6228 | AGAAUUACUAUAAGGUACU | 12582 | AGUACCUUAUAGUAAUUCU |
| siRNA 6229 | 6229 | GAAUUACUAUAAGGUACUG | 12583 | CAGUACCUUAUAGUAAUUC |
| siRNA 6234 | 6234 | ACUAUAAGGUACUGUGAAA | 12588 | UUUCACAGUACCUUAUAGU |
| siRNA 6239 | 6239 | AAGGUACUGUGAAAUAACU | 12593 | AGUUAUUUCACAGUACCUU |
| siRNA 6243 | 6243 | UACUGUGAAAUAACUGCGA | 12597 | UCGCAGUUAUUUCACAGUA |
| siRNA 6244 | 6244 | ACUGUGAAAUAACUGCGAU | 12598 | AUCGCAGUUAUUUCACAGU |
| siRNA 6245 | 6245 | CUGUGAAAUAACUGCGAUU | 12599 | AAUCGCAGUUAUUUCACAG |
| siRNA 6246 | 6246 | UGUGAAAUAACUGCGAUUU | 12600 | AAAUCGCAGUUAUUUCACA |
| siRNA 6248 | 6248 | UGAAAUAACUGCGAUUUUG | 12602 | CAAAAUCGCAGUUAUUUCA |
| siRNA 6258 | 6258 | GCGAUUUUGUGAGCAAAAC | 12612 | GUUUUGCUCACAAAAUCGC |
| siRNA 6267 | 6267 | UGAGCAAAACAUACUUGGA | 12621 | UCCAAGUAUGUUUUGCUCA |
| siRNA 6282 | 6282 | UGGAAAUGCUGAUUGAUUU | 12636 | AAAUCAAUCAGCAUUUCCA |
| siRNA 6290 | 6290 | CUGAUUGAUUUUUAUGCUU | 12644 | AAGCAUAAAAAUCAAUCAG |
| siRNA 6296 | 6296 | GAUUUUUAUGCUUGUUAGU | 12650 | ACUAACAAGCAUAAAAAUC |
| siRNA 6299 | 6299 | UUUUAUGCUUGUUAGUGUA | 12653 | UACACUAACAAGCAUAAAA |
| siRNA 6300 | 6300 | UUUAUGCUUGUUAGUGUAU | 12654 | AUACACUAACAAGCAUAAA |
| siRNA 6314 | 6314 | UGUAUUGCAAGAAACACAG | 12668 | CUGUGUUUCUUGCAAUACA |
| siRNA 6330 | 6330 | CAGAAAAUGUAGUUUUGUU | 12684 | AACAAAACUACAUUUUCUG |
| siRNA 6349 | 6349 | UUAAUAAACCAAAAAUUGA | 12703 | UCAAUUUUUGGUUUAUUAA |

The siRNAs in subset A had the following characteristics: Cross-reactivity: With 19mer in human GPAM mRNA; Specificity category: For human: AS2 or better, SS3 or better; and miRNA seeds: AS+SS strand: seed region not conserved in human, mouse, and rat and not present in >4 species, Off-target frequency: ≤30 human off-targets matched with 2 mismatches in antisense strand; and SNPs: siRNA target sites do not harbor SNPs with a MAF≥1% (pos. 2-18.)

The siRNA sequences in subset A were selected for more stringent specificity to yield subset B. Subset B included the following 1344 siRNAs: 13, 18, 23, 26, 28, 54, 62, 79, 81, 82, 83, 84, 88, 90, 91, 94, 95, 96, 98, 100, 101, 106, 107, 108, 109, 110, 111, 113, 114, 118, 119, 127, 161, 162, 170, 173, 175, 176, 177, 179, 181, 190, 191, 194, 202, 206, 207, 211, 216, 218, 221, 232, 241, 246, 247, 257, 260, 261, 262, 263, 264, 265, 267, 275, 278, 279, 280, 284, 303, 331, 354, 356, 357, 358, 359, 360, 361, 362, 363, 364, 365, 370, 386, 397, 401, 403, 417, 425, 427, 432, 435, 436, 444, 450, 454, 457, 458, 487, 493, 494, 495, 496, 497, 498, 499, 501, 506, 507, 510, 511, 515, 517, 518, 519, 520, 521, 522, 532, 536, 537, 542, 543, 544, 547, 548, 549, 550, 551, 554, 562, 564, 565, 596, 598, 601, 603, 613, 643, 644, 646, 647, 664, 671, 672, 673, 674, 675, 676, 679, 681, 683, 703, 723, 730, 731, 733, 736, 737, 738, 739, 740, 741, 742, 744, 745, 746, 748, 753, 756, 760, 765, 766, 770, 771, 777, 781, 790, 809, 814, 819, 820, 824, 826, 834, 835, 839, 843, 845, 846, 850, 851, 852, 853, 854, 855, 857, 859, 867, 874, 878, 879, 880, 887, 889, 891, 892, 893, 894, 895, 896, 933, 939, 944, 945, 956, 959, 960, 963, 964, 966, 974, 984, 991, 993, 994, 997, 998, 1000, 1001, 1005, 1006, 1018, 1019, 1021, 1022, 1023, 1024, 1025, 1026, 1029, 1030, 1031, 1032, 1033, 1034, 1035, 1044, 1046, 1048, 1052, 1054, 1058, 1065, 1071, 1072, 1079, 1084, 1086, 1087, 1091, 1092, 1093, 1096, 1097, 1098, 1099, 1100, 1102, 1105, 1106, 1111, 1113, 1121, 1122, 1134, 1135, 1136, 1140, 1141, 1142, 1145, 1151, 1152, 1166, 1167, 1169, 1177, 1180, 1181, 1191, 1194, 1196, 1197, 1199, 1202, 1207, 1208, 1209, 1210, 1211, 1212, 1213, 1217, 1230, 1231, 1233, 1236, 1241, 1243, 1245, 1246, 1249, 1252, 1255, 1256, 1257, 1259, 1260, 1263, 1264, 1265, 1266, 1267, 1268, 1269, 1270, 1272, 1279, 1281, 1283, 1285, 1286, 1288, 1289, 1297, 1299, 1300, 1301, 1305, 1308, 1329, 1330, 1333, 1339, 1340, 1341, 1343, 1346, 1350, 1352, 1354, 1355, 1358, 1359, 1361, 1363, 1367, 1373, 1374, 1426, 1432, 1434, 1436, 1461, 1464, 1466, 1467, 1468, 1469, 1470, 1472, 1473, 1474, 1476, 1477, 1478, 1479, 1480, 1481, 1482, 1483, 1484, 1488, 1525, 1530, 1531, 1533, 1534, 1537, 1538, 1539, 1540, 1542, 1543, 1546, 1565, 1566, 1569, 1570, 1571, 1572, 1574, 1577, 1578, 1580, 1582, 1584, 1586, 1600, 1608, 1613, 1614, 1615, 1617, 1618, 1619, 1622, 1625, 1634, 1635, 1639, 1645, 1647, 1650, 1651, 1655, 1656, 1686, 1688, 1689, 1693, 1697, 1702, 1704, 1705, 1706, 1707, 1708, 1709, 1710, 1733, 1736, 1742, 1744, 1746, 1753, 1754, 1757, 1779, 1784, 1786, 1795, 1818, 1824, 1829, 1831, 1832, 1868, 1869, 1871, 1872, 1873, 1877, 1880, 1885, 1886, 1887, 1890, 1891, 1892, 1894, 1896, 1899, 1911, 1915, 1917, 1918, 1919, 1920, 1926, 1930, 1934, 1938, 1939, 1940, 1945, 1946, 1949, 1950, 1955, 1957, 1962, 1963, 1973, 1975, 1976, 1977, 1978, 1982, 1992, 1993, 1996, 1997, 1999, 2001, 2006, 2007, 2008, 2010, 2012, 2017, 2055, 2067, 2076, 2077, 2078, 2082, 2093, 2105, 2110, 2112, 2125, 2126, 2131, 2141, 2142, 2143, 2146, 2158, 2160, 2166, 2167, 2170, 2171, 2182, 2187, 2190, 2192, 2193, 2199, 2205, 2294, 2295, 2296, 2297, 2299, 2301, 2302, 2303, 2304, 2314, 2324, 2332, 2340, 2344, 2345, 2366, 2367, 2378, 2407, 2415, 2427, 2435, 2436, 2437, 2440, 2449, 2453, 2461, 2462, 2463, 2464, 2465, 2466, 2468, 2486, 2494, 2496, 2498, 2504, 2507, 2511, 2515, 2518, 2523, 2524, 2557, 2561, 2564, 2574, 2575, 2597, 2600, 2603, 2618, 2619, 2620, 2626, 2627, 2628, 2629, 2630, 2631, 2632, 2633, 2635, 2637, 2641, 2642, 2649, 2656, 2660, 2661, 2674, 2676, 2678, 2679, 2680, 2681, 2683, 2684, 2692, 2702, 2708, 2711, 2713, 2725, 2726, 2727, 2740, 2816, 2818, 2826, 2829, 2834, 2855, 2872, 2878, 2880, 2883, 2886, 2896, 2898, 2899, 2905, 2906, 2907, 2909, 2910, 2911, 2912, 2913, 2914, 2915, 2916, 2917, 2918, 2919, 2920, 2921, 2922, 2924, 2925, 2926, 2927, 2928, 2931, 2939, 3003, 3004, 3005, 3007, 3033, 3046, 3047, 3048, 3049, 3052, 3054, 3055, 3056, 3057, 3058, 3059, 3073, 3078, 3080, 3081, 3086, 3090, 3093, 3097, 3100, 3106, 3111, 3131, 3159, 3162, 3163, 3164, 3165, 3166, 3168, 3169, 3172, 3181, 3185, 3189, 3190, 3191, 3192, 3194, 3195, 3205, 3219, 3233, 3234, 3238, 3239, 3241, 3244, 3246, 3247, 3265, 3268, 3269, 3274, 3305, 3316, 3317, 3318, 3321, 3322, 3323, 3324, 3325, 3326, 3327, 3330, 3331, 3333, 3334, 3338, 3341, 3358, 3365, 3370, 3374, 3380, 3382, 3383, 3388, 3404, 3406, 3409, 3411, 3425, 3429, 3431, 3434, 3439, 3440, 3445, 3446, 3447, 3452, 3454, 3464, 3465, 3466, 3467, 3468, 3470, 3480, 3481, 3487, 3498, 3503, 3504, 3508, 3516, 3518, 3520, 3522, 3523, 3524, 3526, 3537, 3556, 3559, 3563, 3571, 3572, 3574, 3575, 3576, 3583, 3584, 3591, 3593, 3594, 3595, 3598, 3599, 3606, 3614, 3616, 3620, 3646, 3647, 3656, 3659, 3662, 3667, 3670, 3671, 3672, 3674, 3677, 3682, 3683, 3684, 3699, 3701, 3704, 3709, 3710, 3711, 3714, 3715, 3716, 3717, 3718, 3719, 3731, 3733, 3734, 3740, 3746, 3749, 3752, 3753, 3757, 3758, 3759, 3761, 3763, 3765, 3766, 3771, 3773, 3780, 3808, 3810, 3829, 3832, 3836, 3840, 3842, 3845, 3848, 3849, 3853, 3854, 3859, 3861, 3863, 3864, 3865, 3866, 3871, 3874, 3896, 3898, 3905, 3926, 3931, 3933, 3934, 3986, 3991, 3995, 3996, 3997, 3998, 4004, 4005, 4010, 4011, 4016, 4019, 4022, 4024, 4025, 4053, 4057, 4058, 4073, 4087, 4091, 4095, 4099, 4101, 4106, 4115, 4116, 4142, 4146, 4148, 4149, 4151, 4179, 4180, 4186, 4197, 4207, 4211, 4218, 4220, 4227, 4256, 4262, 4265, 4319, 4324, 4325, 4327, 4330, 4333, 4334, 4335, 4336, 4361, 4377, 4381, 4383, 4384, 4385, 4389, 4400, 4401, 4403, 4418, 4419, 4428, 4429, 4430, 4437, 4441, 4448, 4467, 4468, 4478, 4479, 4506, 4508, 4510, 4511, 4512, 4517, 4518, 4519, 4520, 4521, 4525, 4526, 4529, 4531, 4532, 4535, 4536, 4537, 4538, 4540, 4541, 4543, 4548, 4549, 4551, 4552, 4555, 4564, 4577, 4600, 4602, 4603, 4604, 4615, 4621, 4623, 4663, 4665, 4666, 4667, 4669, 4670, 4671, 4672, 4673, 4675, 4681, 4687, 4694, 4695, 4708, 4709, 4710, 4713, 4718, 4723, 4725, 4727, 4732, 4734, 4735, 4737, 4768, 4771, 4772, 4804, 4805, 4812, 4814, 4821, 4823, 4825, 4830, 4838, 4844, 4856, 4873, 4917, 4918, 4919, 4929, 4931, 4932, 4934, 4936, 4937, 4942, 4943, 4947, 4948, 4949, 4973, 4993, 4994, 4996, 4997, 5002, 5003, 5005, 5009, 5011, 5012, 5013, 5016, 5031, 5036, 5037, 5039, 5040, 5042, 5043, 5044, 5046, 5047, 5052, 5053, 5067, 5082, 5093, 5101, 5125, 5156, 5162, 5168, 5170, 5192, 5194, 5202, 5205, 5218, 5233, 5240, 5242, 5244, 5246, 5251, 5279, 5301, 5312, 5314, 5318, 5340, 5349, 5353, 5359, 5360, 5364, 5385, 5395, 5403, 5411, 5413, 5417, 5421, 5422, 5430, 5432, 5441, 5462, 5463, 5477, 5485, 5486, 5487, 5497, 5498, 5500, 5505, 5507, 5508, 5509, 5510, 5512, 5513, 5514, 5515, 5516, 5517, 5521, 5522, 5523, 5526, 5527, 5528, 5529, 5531, 5532, 5533, 5535, 5536, 5539, 5540, 5541, 5542, 5545, 5547, 5548, 5556, 5557, 5560, 5565, 5567, 5568, 5572, 5573, 5574, 5575, 5578, 5580, 5581, 5601, 5603, 5604, 5605, 5608, 5610, 5614, 5617, 5618, 5622, 5623, 5625, 5626, 5630, 5631, 5633, 5643, 5650, 5651, 5660, 5661, 5662, 5663, 5664, 5673, 5679, 5680, 5681, 5682, 5683, 5687, 5688, 5689, 5693, 5694, 5696, 5701, 5712, 5714, 5716, 5718, 5724, 5727, 5728, 5733, 5734, 5739, 5740, 5741, 5746, 5748, 5749, 5751, 5752, 5753, 5754, 5763, 5768, 5782, 5798, 5800, 5801, 5802, 5803, 5805, 5807, 5810, 5811, 5812, 5813, 5817, 5819, 5820, 5830, 5833, 5835, 5839, 5849, 5865, 5866, 5867, 5868, 5872, 5873, 5875, 5891, 5892, 5902, 5917, 5959, 5964, 5965, 5966, 5967, 5968, 5971, 5972, 5973, 5974, 5977, 5983, 5984, 5986, 5987, 5988, 5990, 5992, 5993, 5994, 5995, 5996, 5997, 5998, 5999, 6001, 6005, 6007, 6010, 6012, 6016, 6018, 6028, 6029, 6032, 6056, 6061, 6063, 6065, 6067, 6070, 6071, 6078, 6098, 6113, 6132, 6133, 6134, 6136, 6137, 6138, 6139, 6141, 6142, 6145, 6146, 6148, 6169, 6170, 6177, 6178, 6179, 6184, 6189, 6192, 6204, 6205, 6209, 6211, 6212, 6219, 6220, 6228, 6229, 6234, 6239, 6243, 6244, 6245, 6246, 6248, 6258, 6267, 6282, 6290, 6296, 6299, 6300, 6314, 6330, and 6349.

The siRNAs in subset B had the following characteristics: Cross-reactivity: With 19mer in human GPAM mRNA; Specificity category: For human: AS2 or better, SS3 or better; miRNA seeds: AS+SS strand: seed region not conserved in human, mouse, and rat and not present in >4 species; Off-target frequency: ≤20 human off-targets matched with 2 mismatches in antisense strand; and SNPs: siRNA target sites do not harbor SNPs with a MAF≥1% (pos. 2-18).

The siRNA sequences in subset B were further selected for absence of seed regions in the AS strand that are identical to a seed region of known human miRNA to yield subset C. Subset C included the following 931 siRNAs: 18, 23, 62, 79, 81, 82, 83, 84, 88, 90, 94, 95, 96, 98, 101, 106, 107, 108, 110, 113, 114, 118, 119, 127, 173, 175, 190, 191, 194, 207, 211, 218, 221, 232, 241, 257, 260, 261, 262, 263, 264, 265, 278, 280, 284, 331, 354, 357, 358, 359, 360, 361, 363, 364, 365, 386, 432, 435, 444, 450, 454, 458, 487, 493, 494, 495, 496, 497, 498, 499, 501, 506, 507, 515, 517, 518, 519, 520, 521, 537, 542, 543, 547, 549, 550, 551, 554, 562, 564, 565, 596, 601, 603, 613, 643, 646, 647, 664, 671, 673, 674, 675, 679, 681, 683, 703, 730, 733, 736, 737, 741, 742, 745, 746, 748, 753, 756, 760, 770, 777, 781, 790, 814, 819, 826, 839, 843, 845, 846, 850, 852, 854, 855, 857, 859, 867, 874, 878, 879, 887, 889, 891, 892, 893, 894, 895, 896, 933, 939, 944, 956, 959, 963, 966, 974, 991, 993, 994, 997, 998, 1006, 1018, 1019, 1024, 1025, 1026, 1029, 1030, 1031, 1032, 1033, 1035, 1044, 1046, 1052, 1054, 1058, 1065, 1071, 1079, 1084, 1086, 1087, 1092, 1093, 1097, 1098, 1099, 1100, 1105, 1106, 1111, 1113, 1135, 1136, 1140, 1145, 1151, 1152, 1166, 1169, 1180, 1181, 1191, 1194, 1196, 1197, 1199, 1202, 1207, 1208, 1209, 1210, 1211, 1212, 1230, 1241, 1243, 1246, 1252, 1255, 1256, 1257, 1259, 1260, 1263, 1264, 1265, 1266, 1267, 1268, 1269, 1285, 1286, 1288, 1289, 1297, 1300, 1308, 1330, 1333, 1339, 1340, 1341, 1343, 1346, 1352, 1354, 1355, 1358, 1359, 1361, 1363, 1367, 1373, 1374, 1426, 1432, 1434, 1436, 1461, 1464, 1466, 1467, 1468, 1470, 1472, 1473, 1476, 1477, 1479, 1480, 1481, 1482, 1484, 1525, 1530, 1531, 1533, 1534, 1537, 1538, 1539, 1543, 1546, 1565, 1566, 1569, 1571, 1577, 1578, 1580, 1582, 1584, 1586, 1600, 1608, 1613, 1617, 1618, 1619, 1625, 1634, 1635, 1645, 1647, 1651, 1655, 1686, 1689, 1702, 1704, 1705, 1706, 1708, 1710, 1742, 1753, 1757, 1779, 1786, 1795, 1824, 1831, 1868, 1869, 1871, 1873, 1877, 1880, 1885, 1886, 1887, 1890, 1891, 1894, 1899, 1917, 1919, 1920, 1926, 1930, 1938, 1945, 1946, 1950, 1955, 1957, 1962, 1963, 1973, 1978, 1982, 1997, 1999, 2008, 2010, 2012, 2017, 2055, 2067, 2077, 2078, 2082, 2093, 2105, 2110, 2125, 2126, 2131, 2141, 2142, 2166, 2167, 2170, 2171, 2182, 2187, 2192, 2193, 2294, 2295, 2299, 2301, 2303, 2304, 2314, 2332, 2340, 2345, 2367, 2407, 2427, 2435, 2436, 2437, 2440, 2453, 2462, 2463, 2464, 2465, 2466, 2486, 2494, 2496, 2504, 2511, 2515, 2518, 2524, 2574, 2575, 2618, 2620, 2627, 2628, 2629, 2630, 2631, 2632, 2633, 2635, 2637, 2660, 2674, 2676, 2678, 2679, 2680, 2681, 2708, 2713, 2725, 2726, 2727, 2808, 2826, 2855, 2872, 2883, 2886, 2896, 2898, 2899, 2905, 2906, 2907, 2909, 2910, 2911, 2912, 2913, 2915, 2916, 2918, 2920, 2921, 2924, 2925, 2926, 2928, 2931, 2939, 3003, 3004, 3007, 3033, 3046, 3049, 3052, 3054, 3055, 3056, 3057, 3058, 3059, 3073, 3078, 3080, 3086, 3090, 3093, 3100, 3106, 3111, 3159, 3162, 3163, 3164, 3165, 3166, 3168, 3169, 3172, 3181, 3185, 3190, 3191, 3194, 3195, 3244, 3316, 3317, 3321, 3322, 3323, 3324, 3325, 3327, 3331, 3333, 3334, 3338, 3341, 3358, 3365, 3370, 3380, 3382, 3383, 3404, 3425, 3429, 3434, 3439, 3440, 3445, 3446, 3454, 3465, 3466, 3467, 3468, 3480, 3487, 3496, 3498, 3503, 3504, 3516, 3518, 3524, 3537, 3556, 3563, 3571, 3572, 3574, 3575, 3584, 3591, 3593, 3594, 3595, 3606, 3616, 3646, 3647, 3656, 3659, 3662, 3667, 3671, 3672, 3674, 3677, 3682, 3683, 3690, 3699, 3704, 3709, 3710, 3714, 3716, 3718, 3719, 3733, 3734, 3740, 3746, 3752, 3753, 3757, 3759, 3761, 3763, 3780, 3808, 3810, 3829, 3832, 3836, 3842, 3849, 3859, 3861, 3863, 3864, 3865, 3871, 3874, 3896, 3898, 3926, 3933, 3934, 3986, 3991, 3996, 3998, 4005, 4010, 4019, 4024, 4025, 4053, 4058, 4091, 4095, 4099, 4106, 4115, 4116, 4142, 4146, 4149, 4151, 4186, 4197, 4207, 4211, 4218, 4220, 4227, 4256, 4262, 4265, 4319, 4324, 4325, 4327, 4330, 4334, 4336, 4361, 4383, 4384, 4385, 4401, 4403, 4429, 4430, 4437, 4441, 4467, 4468, 4478, 4479, 4506, 4510, 4511, 4518, 4519, 4520, 4521, 4525, 4529, 4531, 4532, 4536, 4537, 4538, 4540, 4548, 4549, 4551, 4552, 4600, 4602, 4603, 4604, 4615, 4621, 4663, 4665, 4666, 4667, 4669, 4672, 4675, 4687, 4694, 4695, 4708, 4709, 4713, 4725, 4732, 4734, 4737, 4771, 4805, 4812, 4814, 4830, 4844, 4856, 4873, 4917, 4918, 4919, 4929, 4931, 4932, 4936, 4947, 4948, 4949, 4993, 4994, 4996, 4997, 5002, 5003, 5005, 5011, 5012, 5013, 5016, 5031, 5036, 5039, 5040, 5042, 5043, 5044, 5047, 5052, 5053, 5093, 5101, 5125, 5156, 5168, 5170, 5192, 5218, 5233, 5242, 5244, 5251, 5301, 5312, 5349, 5353, 5359, 5360, 5364, 5403, 5421, 5422, 5432, 5441, 5462, 5463, 5477, 5485, 5486, 5498, 5500, 5507, 5508, 5509, 5512, 5513, 5515, 5516, 5521, 5526, 5528, 5529, 5531, 5532, 5535, 5536, 5539, 5540, 5542, 5545, 5547, 5548, 5556, 5557, 5560, 5565, 5567, 5572, 5573, 5575, 5578, 5580, 5581, 5601, 5603, 5604, 5605, 5610, 5614, 5622, 5623, 5630, 5643, 5650, 5651, 5660, 5661, 5662, 5663, 5673, 5679, 5680, 5681, 5682, 5683, 5687, 5689, 5693, 5694, 5696, 5701, 5712, 5716, 5727, 5728, 5734, 5739, 5748, 5749, 5751, 5752, 5798, 5800, 5801, 5802, 5803, 5805, 5807, 5811, 5812, 5819, 5820, 5833, 5839, 5865, 5866, 5867, 5868, 5872, 5875, 5891, 5892, 5902, 5917, 5959, 5964, 5966, 5971, 5972, 5974, 5977, 5983, 5984, 5986, 5987, 5988, 5990, 5992, 5993, 5995, 5996, 5997, 5999, 6001, 6005, 6007, 6010, 6012, 6016, 6018, 6029, 6056, 6061, 6063, 6065, 6067, 6071, 6098, 6113, 6132, 6133, 6134, 6136, 6141, 6145, 6169, 6170, 6177, 6179, 6189, 6192, 6205, 6209, 6219, 6220, 6228, 6234, 6239, 6243, 6244, 6245, 6246, 6258, 6282, 6290, 6296, 6300, 6314, 6330, and 6349.

The siRNAs in subset C had the following characteristics: Cross-reactivity: With 19mer in human GPAM mRNA; Specificity category: For human: AS2 or better, SS3 or better; miRNA seeds: AS+SS strand: seed region not conserved in human, mouse, and rat and not present in >4 species. AS strand: seed region not identical to seed region of known human miRNA; Off-target frequency: ≤30 human off-targets matched with 2 mismatches by antisense strand; and SNPs: siRNA target sites do not harbor SNPs with a MAF≥1% (pos. 2-18).

The siRNA sequences in subset C were also selected for absence of seed regions in the AS or S strands that are identical to a seed region of known human miRNA in addition to having an off-target frequency of ≤30 human off-targets matched with 2 mismatches by antisense strand to yield subset D. Subset D included the following 595 siRNAs: 18, 23, 79, 83, 94, 95, 98, 106, 107, 108, 110, 114, 118, 127, 173, 175, 190, 191, 207, 218, 221, 241, 257, 260,261, 262, 264, 278, 284, 354, 357, 359, 360, 361, 363, 364, 365, 386, 435, 450, 454, 494, 495, 497, 498, 499, 501, 507, 517, 518, 520, 537, 547, 549, 550, 551, 554, 596, 601, 613, 646, 647, 664, 673, 675, 681, 683, 703, 737, 745, 746, 748, 753, 756, 819, 826, 839, 845, 850, 854, 855, 857, 859, 874, 878, 879, 887, 889, 891, 893, 894, 895, 933, 956, 959, 966, 974, 991, 993, 997, 998, 1006, 1024, 1025, 1026, 1029, 1030, 1031, 1033, 1035, 1044, 1046, 1058, 1065, 1071, 1084, 1087, 1092, 1093, 1097, 1098, 1099, 1100, 1106, 1111, 1135, 1145, 1151, 1152, 1191, 1194, 1196, 1197, 1199, 1207, 1208, 1209, 1210, 1212, 1241, 1243, 1246, 1252, 1256, 1257, 1259, 1263, 1264, 1265, 1266, 1267, 1268, 1269, 1285, 1289, 1297, 1300, 1308, 1339, 1340, 1352, 1354, 1355, 1358, 1359, 1361, 1363, 1367, 1373, 1374, 1426, 1432, 1434, 1461, 1472, 1476, 1477, 1479, 1480, 1481, 1482, 1484, 1531, 1534, 1538, 1539, 1543, 1546, 1569, 1571, 1577, 1578, 1580, 1586, 1600, 1608, 1613, 1618, 1619, 1634, 1645, 1651, 1655, 1705, 1706, 1708, 1710, 1742, 1753, 1757, 1779, 1795, 1824, 1831, 1868, 1873, 1877, 1880, 1887, 1890, 1894, 1899, 1917, 1962, 1963, 1973, 1978, 1982, 1999, 2010, 2012, 2017, 2055, 2067, 2078, 2082, 2110, 2131, 2141, 2142, 2166, 2182, 2299, 2301, 2303, 2367, 2407, 2427, 2435, 2440, 2462, 2463, 2464, 2466, 2486, 2494, 2504, 2524, 2618, 2620, 2627, 2629, 2630, 2631, 2632, 2633, 2635, 2637, 2660, 2674, 2676, 2678, 2679, 2725, 2726, 2727, 2855, 2872, 2883, 2886, 2898, 2899, 2905, 2906, 2907, 2909, 2910, 2911, 2912, 2913, 2915, 2918, 2920, 2921, 2925, 2926, 2928, 2931, 2939, 3007, 3033, 3046, 3052, 3054, 3056, 3059, 3073, 3086, 3090, 3093, 3111, 3162, 3163, 3164, 3165, 3166, 3169, 3181, 3185, 3190, 3244, 3317, 3321, 3323, 3324, 3325, 3327, 3331, 3333, 3334, 3338, 3341, 3370, 3380, 3382, 3383, 3404, 3425, 3429, 3434, 3439, 3445, 3446, 3454, 3468, 3496, 3498, 3503, 3504, 3516, 3518, 3524, 3556, 3572, 3574, 3575, 3591, 3593, 3594, 3595, 3616, 3646, 3667, 3672, 3674, 3677, 3683, 3690, 3699, 3709, 3710, 3716, 3719, 3734, 3746, 3752, 3757, 3780, 3829, 3832, 3836, 3859, 3861, 3864, 3871, 3898, 3926, 3934, 3986, 3991, 3996, 3998, 4005, 4010, 4019, 4024, 4053, 4058, 4095, 4099, 4106, 4115, 4142, 4146, 4149, 4186, 4197, 4211, 4220, 4256, 4265, 4327, 4330, 4336, 4361, 4383, 4384, 4401, 4403, 4429, 4441, 4467, 4468, 4506, 4510, 4518, 4519, 4521, 4529, 4540, 4548, 4549, 4600, 4602, 4604, 4621, 4665, 4666, 4667, 4669, 4687, 4708, 4709, 4732, 4771, 4805, 4812, 4814, 4830, 4856, 4917, 4918, 4919, 4929, 4947, 4948, 4949, 4994, 4996, 4997, 5002, 5003, 5005, 5011, 5012, 5016, 5040, 5042, 5043, 5044, 5052, 5053, 5093, 5170, 5244, 5251, 5301, 5349, 5353, 5359, 5360, 5364, 5422, 5441, 5462, 5463, 5485, 5507, 5509, 5513, 5515, 5516, 5521, 5526, 5528, 5531, 5532, 5536, 5539, 5540, 5542, 5547, 5548, 5556, 5557, 5560, 5567, 5572, 5575, 5578, 5580, 5581, 5603, 5604, 5610, 5614, 5630, 5650, 5651, 5661, 5662, 5663, 5673, 5681, 5687, 5693, 5694, 5696, 5701, 5712, 5727, 5728, 5734, 5739, 5751, 5752, 5798, 5800, 5802, 5803, 5805, 5807, 5812, 5819, 5833, 5839, 5865, 5867, 5868, 5875, 5917, 5959, 5966, 5971, 5972, 5974, 5977, 5983, 5984, 5986, 5987, 5988, 5990, 5992, 5993, 5996, 5999, 6001, 6005, 6012, 6016, 6056, 6061, 6063, 6065, 6071, 6113, 6133, 6134, 6136, 6145, 6189, 6209, 6219, 6220, 6228, 6234, 6239, 6246, 6258, 6290, 6296, 6314, 6330, and 6349.

The siRNA sequences in subset D were also selected to have an off-target frequency of ≤20 human off-targets matched with 2 mismatches by antisense strand to yield subset E. Subset E included the following 593 siRNAs: 18, 23, 79, 83, 94, 95, 98, 106, 107, 108, 110, 114,118, 127, 173, 175, 190, 191, 207, 218, 221, 241, 257, 260, 261, 262, 264, 278, 284, 354, 357, 359, 360, 361, 363, 364, 365, 386, 435, 450, 454, 494, 495, 497, 498, 499, 501, 507, 517, 518, 520, 537, 547, 549, 550, 551, 554, 596, 601, 613, 646, 647, 664, 673, 675, 681, 683, 703, 737, 745, 746, 748, 753, 756, 819, 826, 839, 845, 850, 854, 855, 857, 859, 874, 878, 879, 887, 889, 891, 893, 894, 895, 933, 956, 959, 966, 974, 991, 993, 997, 998, 1006, 1024, 1025, 1026, 1029, 1030, 1031, 1033, 1035, 1044, 1046, 1058, 1065, 1071, 1084, 1087, 1092, 1093, 1097, 1098, 1099, 1100, 1106, 1111, 1135, 1145, 1151, 1152, 1191, 1194, 1196, 1197, 1199, 1207, 1208, 1209, 1210, 1212, 1241, 1243, 1246, 1252, 1256, 1257, 1259, 1263, 1264, 1265, 1266, 1267, 1268, 1269, 1285, 1289, 1297, 1300, 1308, 1339, 1340, 1352, 1354, 1355, 1358, 1359, 1361, 1363, 1367, 1373, 1374, 1426, 1432, 1434, 1461, 1472, 1476, 1477, 1479, 1480, 1481, 1482, 1484, 1531, 1534, 1538, 1539, 1543, 1546, 1569, 1571, 1577, 1578, 1580, 1586, 1600, 1608, 1613, 1618, 1619, 1634, 1645, 1651, 1655, 1705, 1706, 1708, 1710, 1742, 1753, 1757, 1779, 1795, 1824, 1831, 1868, 1873, 1877, 1880, 1887, 1890, 1894, 1899, 1917, 1962, 1963, 1973, 1978, 1982, 1999, 2010, 2012, 2017, 2055, 2067, 2078, 2082, 2110, 2131, 2141, 2142, 2166, 2182, 2299, 2301, 2303, 2367, 2407, 2427, 2435, 2440, 2462, 2463, 2464, 2466, 2486, 2494, 2504, 2524, 2618, 2620, 2627, 2629, 2630, 2631, 2632, 2633, 2635, 2637, 2660, 2674, 2676, 2678, 2679, 2725, 2726, 2727, 2855, 2872, 2883, 2886, 2898, 2899, 2905, 2906, 2907, 2909, 2910, 2911, 2912, 2913, 2915, 2918, 2920, 2921, 2925, 2926, 2928, 2931, 2939, 3007, 3033, 3046, 3052, 3054, 3056, 3059, 3073, 3086, 3090, 3093, 3111, 3162, 3163, 3164, 3165, 3166, 3169, 3181, 3185, 3190, 3244, 3317, 3321, 3323, 3324, 3325, 3327, 3331, 3333, 3334, 3338, 3341, 3370, 3380, 3382, 3383, 3404, 3425, 3429, 3434, 3439, 3445, 3446, 3454, 3468, 3498, 3503, 3504, 3516, 3518, 3524, 3556, 3572, 3574, 3575, 3591, 3593, 3594, 3595, 3616, 3646, 3667, 3672, 3674, 3677, 3683, 3699, 3709, 3710, 3716, 3719, 3734, 3746, 3752, 3757, 3780, 3829, 3832, 3836, 3859, 3861, 3864, 3871, 3898, 3926, 3934, 3986, 3991, 3996, 3998, 4005, 4010, 4019, 4024, 4053, 4058, 4095, 4099, 4106, 4115, 4142, 4146, 4149, 4186, 4197, 4211, 4220, 4256, 4265, 4327, 4330, 4336, 4361, 4383, 4384, 4401, 4403, 4429, 4441, 4467, 4468, 4506, 4510, 4518, 4519, 4521, 4529, 4540, 4548, 4549, 4600, 4602, 4604, 4621, 4665, 4666, 4667, 4669, 4687, 4708, 4709, 4732, 4771, 4805, 4812, 4814, 4830, 4856, 4917, 4918, 4919, 4929, 4947, 4948, 4949, 4994, 4996, 4997, 5002, 5003, 5005, 5011, 5012, 5016, 5040, 5042, 5043, 5044, 5052, 5053, 5093, 5170, 5244, 5251, 5301, 5349, 5353, 5359, 5360, 5364, 5422, 5441, 5462, 5463, 5485, 5507, 5509, 5513, 5515, 5516, 5521, 5526, 5528, 5531, 5532, 5536, 5539, 5540, 5542, 5547, 5548, 5556, 5557, 5560, 5567, 5572, 5575, 5578, 5580, 5581, 5603, 5604, 5610, 5614, 5630, 5650, 5651, 5661, 5662, 5663, 5673, 5681, 5687, 5693, 5694, 5696, 5701, 5712, 5727, 5728, 5734, 5739, 5751, 5752, 5798, 5800, 5802, 5803, 5805, 5807, 5812, 5819, 5833, 5839, 5865, 5867, 5868, 5875, 5917, 5959, 5966, 5971, 5972, 5974, 5977, 5983, 5984, 5986, 5987, 5988, 5990, 5992, 5993, 5996, 5999, 6001, 6005, 6012, 6016, 6056, 6061, 6063, 6065, 6071, 6113, 6133, 6134, 6136, 6145, 6189, 6209, 6219, 6220, 6228, 6234, 6239, 6246, 6258, 6290, 6296, 6314, 6330, and 6349.

Any siRNA among any of subsets A-E may comprise any modification pattern described herein. If a sequence has a different number of nucleotides in length than a modification pattern, the modification pattern may still be used with the appropriate number of additional nucleotides added 5′ or 3′ to match the number of nucleotides in the modification pattern. For example, if a sense or antisense strand of the siRNA among any of subsets A-E comprises 19 nucleotides, and a modification pattern comprises 21 nucleotides, UU may be added onto the 5′ end of the sense or antisense strand.

Therapeutic siRNAs were designed to target human GPAM as described above and, in some cases, the GPAM sequence of at least one toxicology-relevant species, in this case, the non-human primate (NHP) cynomolgus monkey. The siRNAs included in subset F had the following characteristics: Cross-reactivity: With 19mer in human GPAM mRNA, with 17mer/19mer in NHP GPAM; Specificity category: For human and NHP: AS2 or better, SS3 or better. Subset F included 25 siRNAs whose base sequences are shown in Table 9.

TABLE 9
Sequences and siRNA names in subset F (Screening Set)

| siRNA | SEQ ID | Sense strand | SEQ ID | Antisense strand |
|---|---|---|---|---|
| name | NO: | sequence (5′-3′) | NO: | sequence (5′-3′) |
| siRNA 257 | 257 | AUCAGAAUACAGUGUUGGU | 6611 | ACCAACACUGUAUUCUGAU |
| siRNA 261 | 261 | GAAUACAGUGUUGGUCGAU | 6615 | AUCGACCAACACUGUAUUC |
| siRNA 454 | 454 | CUUUGGGUUUGCGGAAUGU | 6808 | ACAUUCCGCAAACCCAAAG |
| siRNA 562 | 562 | AUAAGGGCAUGUUUGCCAC | 6916 | GUGGCAAACAUGCCCUUAU |
| siRNA 845 | 845 | AGCUGCAACUGAGACGAAU | 7199 | AUUCGUCUCAGUUGCAGCU |
| siRNA 878 | 878 | UCUACCAGUUCAUAGAUCC | 7232 | GGAUCUAUGAACUGGUAGA |
| siRNA 879 | 879 | CUACCAGUUCAUAGAUCCC | 7233 | GGGAUCUAUGAACUGGUAG |
| siRNA 998 | 998 | CUUGAUCCAUAAGCUUGGG | 7352 | CCCAAGCUUAUGGAUCAAG |
| siRNA 1058 | 1058 | ACGGAAAGAUGUUCUCUAU | 7412 | AUAGAGAACAUCUUUCCGU |
| siRNA 1087 | 1087 | UCCAUGGGCAUAUAGUUGA | 7441 | UCAACUAUAUGCCCAUGGA |
| siRNA 1097 | 1097 | UAUAGUUGAAUUACUUCGA | 7451 | UCGAAGUAAUUCAACUAUA |
| siRNA 1098 | 1098 | AUAGUUGAAUUACUUCGAC | 7452 | GUCGAAGUAAUUCAACUAU |
| siRNA 1100 | 1100 | AGUUGAAUUACUUCGACAG | 7454 | CUGUCGAAGUAAUUCAACU |
| siRNA 1111 | 1111 | UUCGACAGCAGCAAUUCUU | 7465 | AAGAAUUGCUGCUGUCGAA |
| siRNA 1199 | 1199 | AGUUGUGGUAGAUACUCUG | 7553 | CAGAGUAUCUACCACAACU |
| siRNA 1256 | 1256 | UGGAAUCUCCUAUGAUCGC | 7610 | GCGAUCAUAGGAGAUUCCA |
| siRNA 1279 | 1279 | UCGAAGGUCACUACAAUGG | 7633 | CCAUUGUAGUGACCUUCGA |
| siRNA 1352 | 1352 | UGUUAUUAGAAUGUUACGA | 7706 | UCGUAACAUUCUAAUAACA |
| siRNA 1373 | 1373 | AAACUAUGGUUGUGUCCGA | 7727 | UCGGACACAACCAUAGUUU |
| siRNA 1477 | 1477 | AAGCGUUGUUACCAGCUAU | 7831 | AUAGCUGGUAACAACGCUU |
| siRNA 1618 | 1618 | UAUUCACUGCUAGCAAGUC | 7972 | GACUUGCUAGCAGUGAAUA |
| siRNA 1619 | 1619 | AUUCACUGCUAGCAAGUCC | 7973 | GGACUUGCUAGCAGUGAAU |
| siRNA 1645 | 1645 | UUAUGUCCACACACAUUGU | 7999 | ACAAUGUGUGUGGACAUAA |
| siRNA 2486 | 2486 | AACAGAAAGAAAUGUUGCA | 8840 | UGCAACAUUUCUUUCUGUU |
| siRNA 2660 | 2660 | AUAUAUUCUGAGUUUUGUG | 9014 | CACAAAACUCAGAAUAUAU |

In some cases, the sense strand of any of the siRNAs of subset F comprises siRNA with a particular modification pattern. In this modification pattern, position 9 counting from the 5′ end of the of the sense strand is has the 2′F modification. If position 9 of the sense strand is a pyrimidine, then all purines in the sense strand have the 2′OMe modification. If position 9 is the only pyrimidine between positions 5 and 11 of the sense stand, then position 9 is the only position with the 2′F modification in the sense strand. If position 9 and only one other base between positions 5 and 11 of the sense strand are pyrimidines, then both of these pyrimidines are the only two positions with the 2′F modification in the sense strand. If position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. If there are >2 pyrimidines between positions 5 and 11 of the sense strand, then all combinations of pyrimidines having the 2′F modification are allowed that have three to five 2′F modifications in total, provided that the sense strand does not have three 2′F modifications in a row.

If position 9 of the sense strand is a purine, then all purines in the sense strand have the 2′OMe modification. If position 9 is the only purine between positions 5 and 11 of the sense stand, then position 9 is the only position with the 2′F modification in the sense strand. If position 9 and only one other base between positions 5 and 11 of the sense strand are purines, then both of these purines are the only two positions with the 2′F modification in the sense strand. If position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. If there are >2 purines between positions 5 and 11 of the sense strand, then all combinations of purines having the 2′F modification are allowed that have three to five 2′F modifications in total, provided that the sense strand does not have three 2′F modifications in a row.

In some cases, position 9 of the sense strand can be a 2′deoxy. In these cases, 2′F and 2′OMe modifications may occur at the other positions of the sense strand.

In some cases, the sense strand of any of the siRNAs of subset F comprises a modification pattern which conforms to these sense strand rules (Table 10).

In some cases, the antisense strand of any of the siRNAs of subset F comprise a modification or modification pattern. Some such examples are included in Table 10. The siRNAs in Table 10 include a GalNAc moiety. Table 11 includes some additional sense strand modifications of the siRNAs in subset F. The siRNAs in subset F may comprise any other modification pattern(s). In the tables, Nf (e.g. Af, Cf, Gf, Tf, or Uf) is a 2′ fluoro-modified nucleoside, dN (e.g. dA, dC, dG, dT, or dU) is a 2′ deoxy nucleoside, n (e.g. a, c, g, t, or u) is a 2′ O-methyl modified nucleoside, and “s” is a phosphorothioate linkage.

TABLE 10
Modified Screening Set (Subset G)

| siRNA | SEQ ID | Sense strand sequence (5′-3′), | SEQ ID | |
|---|---|---|---|---|
| Name | NO: | with GalNAc moiety | NO: | Antisense strand sequence (5′-3′) |
| ETD01994 | 12709 | [ETL17]saucaGfAfauAfcAfguguuggasusu | 12734 | usCfscAfaCfaCfuGfuAfuUfcUfgAfususu |
| ETD01995 | 12710 | [ETL17]sgaauaCfagUfgUfuggucgaasusu | 12735 | usUfscGfaCfcAfaCfaCfuGfuAfuUfcsusu |
| ETD01996 | 12711 | [ETL17]scuuugGfGfUfdTugcggaaugasusu | 12736 | usCfsaUfuCfcGfcAfaAfcCfcAfaAfgsusu |
| ETD01997 | 12712 | [ETL17]sauaaGfGfgcAfuGfuuugccaasusu | 12737 | usUfsgGfcAfaAfcAfuGfcCfcUfuAfususu |
| ETD01998 | 12713 | [ETL17]sagcugCfaaCfUfgagacgaaasusu | 12738 | usUfsuCfgUfcUfcAfgUfuGfcAfgCfususu |
| ETD01999 | 12714 | [ETL17]sucuaCfCfagUfuCfauagaucasusu | 12739 | usGfsaUfcUfaUfgAfaCfuGfgUfaGfasusu |
| ETD02000 | 12715 | [ETL17]scuacCfagUfUfcauagauccasusu | 12740 | usGfsgAfuCfuAfuGfaAfcUfgGfuAfgsusu |
| ETD02001 | 12716 | [ETL17]scuugAfuccAfuAfagcuuggasusu | 12741 | usCfscAfaGfcUfuAfuGfgAfuCfaAfgsusu |
| ETD02002 | 12717 | [ETL17]sacggAfAfaGfAfuGfuucucuaasusu | 12742 | usUfsaGfaGfaAfcAfuCfuUfuCfcGfususu |
| ETD02003 | 12718 | [ETL17]succaUfgggCfaUfauaguugasusu | 12743 | usCfsaAfcUfaUfaUfgCfcCfaUfgGfasusu |
| ETD02004 | 12719 | [ETL17]suauaGfuuGfAfauuacuucgasusu | 12744 | usCfsgAfaGfuAfaUfuCfaAfcUfaUfasusu |
| ETD02005 | 12720 | [ETL17]sauaguUfGfAfdAuuacuucgaasusu | 12745 | usUfscGfaAfgUfaAfuUfcAfaCfuAfususu |
| ETD02006 | 12721 | [ETL17]saguugaaUfUfaCfuucgacaasusu | 12746 | usUfsgUfcGfaAfgUfaAfuUfcAfaCfususu |
| ETD02007 | 12722 | [ETL17]suucgaCfAfGfdCagcaauucuasusu | 12747 | usAfsgAfaUfuGfcUfgCfuGfuCfgAfasusu |
| ETD02008 | 12723 | [ETL17]saguugUfGfGfdTagauacucuasusu | 12748 | usAfsgAfgUfaUfcUfaCfcAfcAfaCfususu |
| ETD02009 | 12724 | [ETL17]suggaaUfCfuCfCfuaugaucgasusu | 12749 | usCfsgAfuCfaUfaGfgAfgAfuUfcCfasusu |
| ETD02010 | 12725 | [ETL17]sucgaaggUfCfaCfuacaaugasusu | 12750 | usCfsaUfuGfuAfgUfgAfcCfuUfcGfasusu |
| ETD02011 | 12726 | [ETL17]suguuAfuuAfGfaAfuguuacgasusu | 12751 | usCfsgUfaAfcAfuUfcUfaAfuAfaCfasusu |
| ETD02012 | 12727 | [ETL17]saaacuAfuGfGfuuguguccgasusu | 12752 | usCfsgGfaCfaCfaAfcCfaUfaGfuUfususu |
| ETD02013 | 12728 | [ETL17]saagcgUfUfgUfUfaccagcuaasusu | 12753 | usUfsaGfcUfgGfuAfaCfaAfcGfcUfususu |
| ETD02014 | 12729 | [ETL17]suauucAfCfUfdGcuagcaaguasusu | 12754 | usAfscUfuGfcUfaGfcAfgUfgAfaUfasusu |
| ETD02015 | 12730 | [ETL17]sauucaCfUfgCfUfagcaagucasusu | 12755 | usGfsaCfuUfgCfuAfgCfaGfuGfaAfususu |
| ETD02016 | 12731 | [ETL17]suuauGfuccAfcAfcacauugasusu | 12756 | usCfsaAfuGfuGfuGfuGfgAfcAfuAfasusu |
| ETD02017 | 12732 | [ETL17]saacaGfAfaAfGfaAfauguugcasusu | 12757 | usGfscAfaCfaUfuUfcUfuUfcUfgUfususu |
| ETD02018 | 12733 | [ETL17]sauauaUfUfCfUfgaguuuuguasusu | 12758 | usAfscAfaAfaCfuCfaGfaAfuAfuAfususu |

TABLE 11
Additional Examples of Modified siRNAs (Subset H)

| siRNA | SEQ ID | | SEQ ID | |
|---|---|---|---|---|
| Name | NO: | Sense strand sequence (5′-3′) | NO: | Antisense strand sequence (5′-3′) |
| siRNA 12759 | 12759 | asuscagaAfuAfcAfguguuggasusu | 12734 | usCfscAfaCfaCfuGfuAfuUfcUfgAfususu |
| siRNA 12760 | 12760 | asuscagAfauAfcAfguguuggasusu | 12734 | usCfscAfaCfaCfuGfuAfuUfcUfgAfususu |
| siRNA 12761 | 12761 | asuscagAfAfuAfcaguguuggasusu | 12734 | usCfscAfaCfaCfuGfuAfuUfcUfgAfususu |
| siRNA 12762 | 12762 | asuscagAfAfuAfcAfguguuggasusu | 12734 | usCfscAfaCfaCfuGfuAfuUfcUfgAfususu |
| siRNA 12763 | 12763 | asuscaGfaauAfcAfguguuggasusu | 12734 | usCfscAfaCfaCfuGfuAfuUfcUfgAfususu |
| siRNA 12764 | 12764 | asuscaGfaAfuAfcaguguuggasusu | 12734 | usCfscAfaCfaCfuGfuAfuUfcUfgAfususu |
| siRNA 12765 | 12765 | asuscaGfaAfuAfcAfguguuggasusu | 12734 | usCfscAfaCfaCfuGfuAfuUfcUfgAfususu |
| siRNA 12766 | 12766 | asuscaGfAfauAfcaguguuggasusu | 12734 | usCfscAfaCfaCfuGfuAfuUfcUfgAfususu |
| siRNA 12767 | 12767 | csusuuggguUfUfgcggaaugasusu | 12736 | usCfsaUfuCfcGfcAfaAfcCfcAfaAfgsusu |
| siRNA 12768 | 12768 | csusuugggUfUfugcggaaugasusu | 12736 | usCfsaUfuCfcGfcAfaAfcCfcAfaAfgsusu |
| siRNA 12769 | 12769 | asusaaggGfcAfuGfuuugccaasusu | 12737 | usUfsgGfcAfaAfcAfuGfcCfcUfuAfususu |
| siRNA 12770 | 12770 | asusaagGfgcAfuGfuuugccaasusu | 12737 | usUfsgGfcAfaAfcAfuGfcCfcUfuAfususu |
| siRNA 12771 | 12771 | asusaagGfGfcAfuguuugccaasusu | 12737 | usUfsgGfcAfaAfcAfuGfcCfcUfuAfususu |
| siRNA 12772 | 12772 | asusaagGfGfcAfuGfuuugccaasusu | 12737 | usUfsgGfcAfaAfcAfuGfcCfcUfuAfususu |
| siRNA 12773 | 12773 | asusaaGfggcAfuGfuuugccaasusu | 12737 | usUfsgGfcAfaAfcAfuGfcCfcUfuAfususu |
| siRNA 12774 | 12774 | asusaaGfgGfcAfuguuugccaasusu | 12737 | usUfsgGfcAfaAfcAfuGfcCfcUfuAfususu |
| siRNA 12775 | 12775 | asusaaGfgGfcAfuGfuuugccaasusu | 12737 | usUfsgGfcAfaAfcAfuGfcCfcUfuAfususu |
| siRNA 12776 | 12776 | asusaaGfGfgcAfuguuugccaasusu | 12737 | usUfsgGfcAfaAfcAfuGfcCfcUfuAfususu |
| siRNA 12777 | 12777 | uscsuacCfagUfuCfauagaucasusu | 12739 | usGfsaUfcUfaUfgAfaCfuGfgUfaGfasusu |
| siRNA 12778 | 12778 | uscsuacCfagUfUfcauagaucasusu | 12739 | usGfsaUfcUfaUfgAfaCfuGfgUfaGfasusu |
| siRNA 12779 | 12779 | uscsuaCfcagUfuCfauagaucasusu | 12739 | usGfsaUfcUfaUfgAfaCfuGfgUfaGfasusu |
| siRNA 12780 | 12780 | uscsuaCfcagUfUfcauagaucasusu | 12739 | usGfsaUfcUfaUfgAfaCfuGfgUfaGfasusu |
| siRNA 12781 | 12781 | uscsuaCfCfagUfucauagaucasusu | 12739 | usGfsaUfcUfaUfgAfaCfuGfgUfaGfasusu |
| siRNA 12782 | 12782 | uscsuaCfCfagUfUfcauagaucasusu | 12739 | usGfsaUfcUfaUfgAfaCfuGfgUfaGfasusu |
| siRNA 12783 | 12783 | csusacCfaguUfCfauagauccasusu | 12740 | usGfsgAfuCfuAfuGfaAfcUfgGfuAfgsusu |
| siRNA 12784 | 12784 | ascsggaaaGfAfuGfuucucuaasusu | 12742 | usUfsaGfaGfaAfcAfuCfuUfuCfcGfususu |
| siRNA 12785 | 12785 | ascsggaaAfgAfuGfuucucuaasusu | 12742 | usUfsaGfaGfaAfcAfuCfuUfuCfcGfususu |
| siRNA 12786 | 12786 | ascsggaAfagAfuGfuucucuaasusu | 12742 | usUfsaGfaGfaAfcAfuCfuUfuCfcGfususu |
| siRNA 12787 | 12787 | ascsggaAfaGfAfuguucucuaasusu | 12742 | usUfsaGfaGfaAfcAfuCfuUfuCfcGfususu |
| siRNA 12788 | 12788 | ascsggaAfaGfAfuGfuucucuaasusu | 12742 | usUfsaGfaGfaAfcAfuCfuUfuCfcGfususu |
| siRNA 12789 | 12789 | ascsggaAfAfgAfuguucucuaasusu | 12742 | usUfsaGfaGfaAfcAfuCfuUfuCfcGfususu |
| siRNA 12790 | 12790 | ascsggaAfAfgAfuGfuucucuaasusu | 12742 | usUfsaGfaGfaAfcAfuCfuUfuCfcGfususu |
| siRNA 12791 | 12791 | ascsggaAfAfGfAfuguucucuaasusu | 12742 | usUfsaGfaGfaAfcAfuCfuUfuCfcGfususu |
| siRNA 12792 | 12792 | ascsggaAfAfGfAfuGfuucucuaasusu | 12742 | usUfsaGfaGfaAfcAfuCfuUfuCfcGfususu |
| siRNA 12793 | 12793 | ascsggAfaagAfuGfuucucuaasusu | 12742 | usUfsaGfaGfaAfcAfuCfuUfuCfcGfususu |
| siRNA 12794 | 12794 | ascsggAfaaGfAfuguucucuaasusu | 12742 | usUfsaGfaGfaAfcAfuCfuUfuCfcGfususu |
| siRNA 12795 | 12795 | ascsggAfaaGfAfuGfuucucuaasusu | 12742 | usUfsaGfaGfaAfcAfuCfuUfuCfcGfususu |
| siRNA 12796 | 12796 | ascsggAfaAfgAfuguucucuaasusu | 12742 | usUfsaGfaGfaAfcAfuCfuUfuCfcGfususu |
| siRNA 12797 | 12797 | ascsggAfaAfgAfuGfuucucuaasusu | 12742 | usUfsaGfaGfaAfcAfuCfuUfuCfcGfususu |
| siRNA 12798 | 12798 | ascsggAfAfagAfuguucucuaasusu | 12742 | usUfsaGfaGfaAfcAfuCfuUfuCfcGfususu |
| siRNA 12799 | 12799 | ascsggAfAfagAfuGfuucucuaasusu | 12742 | usUfsaGfaGfaAfcAfuCfuUfuCfcGfususu |
| siRNA 12800 | 12800 | ascsggAfAfaGfAfuguucucuaasusu | 12742 | usUfsaGfaGfaAfcAfuCfuUfuCfcGfususu |
| siRNA 12801 | 12801 | ascsggAfAfAfGfAfuguucucuaasusu | 12742 | usUfsaGfaGfaAfcAfuCfuUfuCfcGfususu |
| siRNA 12802 | 12802 | usasuaGfuugAfAfuuacuucgasusu | 12744 | usCfsgAfaGfuAfaUfuCfaAfcUfaUfasusu |
| siRNA 12803 | 12803 | asusaguugAfAfuuacuucgaasusu | 12745 | usUfscGfaAfgUfaAfuUfcAfaCfuAfususu |
| siRNA 12804 | 12804 | asusaguuGfaAfuuacuucgaasusu | 12745 | usUfscGfaAfgUfaAfuUfcAfaCfuAfususu |
| siRNA 12805 | 12805 | ususcgaCfagCfagcaauucuasusu | 12747 | usAfsgAfaUfuGfcUfgCfuGfuCfgAfasusu |
| siRNA 12806 | 12806 | usgsgaaucUfCfcUfaugaucgasusu | 12749 | usCfsgAfuCfaUfaGfgAfgAfuUfcCfasusu |
| siRNA 12807 | 12807 | usgsgaaucUfCfCfUfaugaucgasusu | 12749 | usCfsgAfuCfaUfaGfgAfgAfuUfcCfasusu |
| siRNA 12808 | 12808 | usgsgaauCfuCfcUfaugaucgasusu | 12749 | usCfsgAfuCfaUfaGfgAfgAfuUfcCfasusu |
| siRNA 12809 | 12809 | usgsgaauCfuCfCfuaugaucgasusu | 12749 | usCfsgAfuCfaUfaGfgAfgAfuUfcCfasusu |
| siRNA 12810 | 12810 | usgsgaauCfUfCfCfuaugaucgasusu | 12749 | usCfsgAfuCfaUfaGfgAfgAfuUfcCfasusu |
| siRNA 12811 | 12811 | usgsgaauCfUfCfCfUfaugaucgasusu | 12749 | usCfsgAfuCfaUfaGfgAfgAfuUfcCfasusu |
| siRNA 12812 | 12812 | usgsgaaUfcuCfcUfaugaucgasusu | 12749 | usCfsgAfuCfaUfaGfgAfgAfuUfcCfasusu |
| siRNA 12813 | 12813 | usgsgaaUfcuCfCfuaugaucgasusu | 12749 | usCfsgAfuCfaUfaGfgAfgAfuUfcCfasusu |
| siRNA 12814 | 12814 | usgsgaaUfcUfCfcuaugaucgasusu | 12749 | usCfsgAfuCfaUfaGfgAfgAfuUfcCfasusu |
| siRNA 12815 | 12815 | usgsgaaUfcUfCfcUfaugaucgasusu | 12749 | usCfsgAfuCfaUfaGfgAfgAfuUfcCfasusu |
| siRNA 12816 | 12816 | usgsgaaUfcUfCfCfUfaugaucgasusu | 12749 | usCfsgAfuCfaUfaGfgAfgAfuUfcCfasusu |
| siRNA 12817 | 12817 | usgsgaaUfCfuCfcuaugaucgasusu | 12749 | usCfsgAfuCfaUfaGfgAfgAfuUfcCfasusu |
| siRNA 12818 | 12818 | usgsgaaUfCfuCfcUfaugaucgasusu | 12749 | usCfsgAfuCfaUfaGfgAfgAfuUfcCfasusu |
| siRNA 12819 | 12819 | usgsgaaUfCfUfCfcuaugaucgasusu | 12749 | usCfsgAfuCfaUfaGfgAfgAfuUfcCfasusu |
| siRNA 12820 | 12820 | usgsgaaUfCfUfCfcUfaugaucgasusu | 12749 | usCfsgAfuCfaUfaGfgAfgAfuUfcCfasusu |
| siRNA 12821 | 12821 | usgsgaaUfCfUfCfCfuaugaucgasusu | 12749 | usCfsgAfuCfaUfaGfgAfgAfuUfcCfasusu |
| siRNA 12822 | 12822 | usgsuuauuAfGfaAfuguuacgasusu | 12751 | usCfsgUfaAfcAfuUfcUfaAfuAfaCfasusu |
| siRNA 12823 | 12823 | usgsuuauuAfGfAfAfuguuacgasusu | 12751 | usCfsgUfaAfcAfuUfcUfaAfuAfaCfasusu |
| siRNA 12824 | 12824 | usgsuuAfuuaGfaAfuguuacgasusu | 12751 | usCfsgUfaAfcAfuUfcUfaAfuAfaCfasusu |
| siRNA 12825 | 12825 | usgsuuAfuuaGfAfauguuacgasusu | 12751 | usCfsgUfaAfcAfuUfcUfaAfuAfaCfasusu |
| siRNA 12826 | 12826 | usgsuuAfuuAfGfaauguuacgasusu | 12751 | usCfsgUfaAfcAfuUfcUfaAfuAfaCfasusu |
| siRNA 12827 | 12827 | usgsuuAfuuAfGfAfAfuguuacgasusu | 12751 | usCfsgUfaAfcAfuUfcUfaAfuAfaCfasusu |
| siRNA 12828 | 12828 | asasgcguUfgUfUfaccagcuaasusu | 12753 | usUfsaGfcUfgGfuAfaCfaAfcGfcUfususu |
| siRNA 12829 | 12829 | asasgcgUfugUfUfaccagcuaasusu | 12753 | usUfsaGfcUfgGfuAfaCfaAfcGfcUfususu |
| siRNA 12830 | 12830 | asasgcgUfUfgUfuaccagcuaasusu | 12753 | usUfsaGfcUfgGfuAfaCfaAfcGfcUfususu |
| siRNA 12831 | 12831 | asusucacUfgCfUfagcaagucasusu | 12755 | usGfsaCfuUfgCfuAfgCfaGfuGfaAfususu |
| siRNA 12832 | 12832 | asusucaCfugCfUfagcaagucasusu | 12755 | usGfsaCfuUfgCfuAfgCfaGfuGfaAfususu |
| siRNA 12833 | 12833 | asusucaCfUfgCfuagcaagucasusu | 12755 | usGfsaCfuUfgCfuAfgCfaGfuGfaAfususu |
| siRNA 12834 | 12834 | asascagaaAfGfaAfauguugcasusu | 12757 | usGfscAfaCfaUfuUfcUfuUfcUfgUfususu |
| siRNA 12835 | 12835 | asascagaaAfGfAfAfauguugcasusu | 12757 | usGfscAfaCfaUfuUfcUfuUfcUfgUfususu |
| siRNA 12836 | 12836 | asascagaAfaGfaAfauguugcasusu | 12757 | usGfscAfaCfaUfuUfcUfuUfcUfgUfususu |
| siRNA 12837 | 12837 | asascagaAfaGfAfaauguugcasusu | 12757 | usGfscAfaCfaUfuUfcUfuUfcUfgUfususu |
| siRNA 12838 | 12838 | asascagaAfAfGfAfaauguugcasusu | 12757 | usGfscAfaCfaUfuUfcUfuUfcUfgUfususu |
| siRNA 12839 | 12839 | asascagaAfAfGfAfAfauguugcasusu | 12757 | usGfscAfaCfaUfuUfcUfuUfcUfgUfususu |
| siRNA 12840 | 12840 | asascagAfaaGfaAfauguugcasusu | 12757 | usGfscAfaCfaUfuUfcUfuUfcUfgUfususu |
| siRNA 12841 | 12841 | asascagAfaaGfAfaauguugcasusu | 12757 | usGfscAfaCfaUfuUfcUfuUfcUfgUfususu |
| siRNA 12842 | 12842 | asascagAfaAfGfaaauguugcasusu | 12757 | usGfscAfaCfaUfuUfcUfuUfcUfgUfususu |
| siRNA 12843 | 12843 | asascagAfaAfGfaAfauguugcasusu | 12757 | usGfscAfaCfaUfuUfcUfuUfcUfgUfususu |
| siRNA 12844 | 12844 | asascagAfaAfGfAfAfauguugcasusu | 12757 | usGfscAfaCfaUfuUfcUfuUfcUfgUfususu |
| siRNA 12845 | 12845 | asascagAfAfaGfaaauguugcasusu | 12757 | usGfscAfaCfaUfuUfcUfuUfcUfgUfususu |
| siRNA 12846 | 12846 | asascagAfAfaGfaAfauguugcasusu | 12757 | usGfscAfaCfaUfuUfcUfuUfcUfgUfususu |
| siRNA 12847 | 12847 | asascagAfAfaGfAfaauguugcasusu | 12757 | usGfscAfaCfaUfuUfcUfuUfcUfgUfususu |
| siRNA 12848 | 12848 | asascagAfAfAfGfaaauguugcasusu | 12757 | usGfscAfaCfaUfuUfcUfuUfcUfgUfususu |
| siRNA 12849 | 12849 | asascagAfAfAfGfaAfauguugcasusu | 12757 | usGfscAfaCfaUfuUfcUfuUfcUfgUfususu |
| siRNA 12850 | 12850 | asascagAfAfAfGfAfaauguugcasusu | 12757 | usGfscAfaCfaUfuUfcUfuUfcUfgUfususu |
| siRNA 12851 | 12851 | asascaGfaaaGfaAfauguugcasusu | 12757 | usGfscAfaCfaUfuUfcUfuUfcUfgUfususu |
| siRNA 12852 | 12852 | asascaGfaaaGfAfaauguugcasusu | 12757 | usGfscAfaCfaUfuUfcUfuUfcUfgUfususu |
| siRNA 12853 | 12853 | asascaGfaaAfGfaaauguugcasusu | 12757 | usGfscAfaCfaUfuUfcUfuUfcUfgUfususu |
| siRNA 12854 | 12854 | asascaGfaaAfGfaAfauguugcasusu | 12757 | usGfscAfaCfaUfuUfcUfuUfcUfgUfususu |
| siRNA 12855 | 12855 | asascaGfaaAfGfAfAfauguugcasusu | 12757 | usGfscAfaCfaUfuUfcUfuUfcUfgUfususu |
| siRNA 12856 | 12856 | asascaGfaAfaGfaaauguugcasusu | 12757 | usGfscAfaCfaUfuUfcUfuUfcUfgUfususu |
| siRNA 12857 | 12857 | asascaGfaAfaGfaAfauguugcasusu | 12757 | usGfscAfaCfaUfuUfcUfuUfcUfgUfususu |
| siRNA 12858 | 12858 | asascaGfaAfaGfAfaauguugcasusu | 12757 | usGfscAfaCfaUfuUfcUfuUfcUfgUfususu |
| siRNA 12859 | 12859 | asascaGfaAfAfGfAfaauguugcasusu | 12757 | usGfscAfaCfaUfuUfcUfuUfcUfgUfususu |
| siRNA 12860 | 12860 | asascaGfAfaaGfaaauguugcasusu | 12757 | usGfscAfaCfaUfuUfcUfuUfcUfgUfususu |
| siRNA 12861 | 12861 | asascaGfAfaaGfaAfauguugcasusu | 12757 | usGfscAfaCfaUfuUfcUfuUfcUfgUfususu |
| siRNA 12862 | 12862 | asascaGfAfaaGfAfaauguugcasusu | 12757 | usGfscAfaCfaUfuUfcUfuUfcUfgUfususu |
| siRNA 12863 | 12863 | asascaGfAfaAfGfaaauguugcasusu | 12757 | usGfscAfaCfaUfuUfcUfuUfcUfgUfususu |
| siRNA 12864 | 12864 | asascaGfAfAfAfGfaaauguugcasusu | 12757 | usGfscAfaCfaUfuUfcUfuUfcUfgUfususu |
| siRNA 12865 | 12865 | asusauaUfuCfUfgaguuuuguasusu | 12758 | usAfscAfaAfaCfuCfaGfaAfuAfuAfususu |
| siRNA 12866 | 12866 | asusauaUfUfcUfgaguuuuguasusu | 12758 | usAfscAfaAfaCfuCfaGfaAfuAfuAfususu |

The siRNAs targeting human GPAM can also be selected using other criteria including cross-reactivity with the NHP cynomolgus GPAM, and in some cases mouse GPAM. Selection of these siRNAs yields Subset I. Subset I includes 25 siRNAs whose base sequences are shown in Table 11A.

TABLE 11A
Subset I siRNAs

| | Sense | | Antisense | |
|---|---|---|---|---|
| | strand | | strand | |
| siRNA | SEQ ID | Sense strand | SEQ ID | Antisense strand |
| Name | NO: | sequence (5′-3′) | NO: | sequence (5′-3′) |
| siRNA 257 | 257 | AUCAGAAUACAGUGUUGGU | 6611 | ACCAACACUGUAUUCUGAU |
| siRNA 261 | 261 | GAAUACAGUGUUGGUCGAU | 6615 | AUCGACCAACACUGUAUUC |
| siRNA 454 | 454 | CUUUGGGUUUGCGGAAUGU | 6808 | ACAUUCCGCAAACCCAAAG |
| siRNA 562 | 562 | AUAAGGGCAUGUUUGCCAC | 6916 | GUGGCAAACAUGCCCUUAU |
| siRNA 845 | 845 | AGCUGCAACUGAGACGAAU | 7199 | AUUCGUCUCAGUUGCAGCU |
| siRNA 878 | 878 | UCUACCAGUUCAUAGAUCC | 7232 | GGAUCUAUGAACUGGUAGA |
| siRNA 879 | 879 | CUACCAGUUCAUAGAUCCC | 7233 | GGGAUCUAUGAACUGGUAG |
| siRNA 998 | 998 | CUUGAUCCAUAAGCUUGGG | 7352 | CCCAAGCUUAUGGAUCAAG |
| siRNA 1058 | 1058 | ACGGAAAGAUGUUCUCUAU | 7412 | AUAGAGAACAUCUUUCCGU |
| siRNA 1087 | 1087 | UCCAUGGGCAUAUAGUUGA | 7441 | UCAACUAUAUGCCCAUGGA |
| siRNA 1097 | 1097 | UAUAGUUGAAUUACUUCGA | 7451 | UCGAAGUAAUUCAACUAUA |
| siRNA 1098 | 1098 | AUAGUUGAAUUACUUCGAC | 7452 | GUCGAAGUAAUUCAACUAU |
| siRNA 1100 | 1100 | AGUUGAAUUACUUCGACAG | 7454 | CUGUCGAAGUAAUUCAACU |
| siRNA 1111 | 1111 | UUCGACAGCAGCAAUUCUU | 7465 | AAGAAUUGCUGCUGUCGAA |
| siRNA 1199 | 1199 | AGUUGUGGUAGAUACUCUG | 7553 | CAGAGUAUCUACCACAACU |
| siRNA 1256 | 1256 | UGGAAUCUCCUAUGAUCGC | 7610 | GCGAUCAUAGGAGAUUCCA |
| siRNA 1279 | 1279 | UCGAAGGUCACUACAAUGG | 7633 | CCAUUGUAGUGACCUUCGA |
| siRNA 1352 | 1352 | UGUUAUUAGAAUGUUACGA | 7706 | UCGUAACAUUCUAAUAACA |
| siRNA 1373 | 1373 | AAACUAUGGUUGUGUCCGA | 7727 | UCGGACACAACCAUAGUUU |
| siRNA 1477 | 1477 | AAGCGUUGUUACCAGCUAU | 7831 | AUAGCUGGUAACAACGCUU |
| siRNA 1618 | 1618 | UAUUCACUGCUAGCAAGUC | 7972 | GACUUGCUAGCAGUGAAUA |
| siRNA 1619 | 1619 | AUUCACUGCUAGCAAGUCC | 7973 | GGACUUGCUAGCAGUGAAU |
| siRNA 1645 | 1645 | UUAUGUCCACACACAUUGU | 7999 | ACAAUGUGUGUGGACAUAA |
| siRNA 2486 | 2486 | AACAGAAAGAAAUGUUGCA | 8840 | UGCAACAUUUCUUUCUGUU |
| siRNA 2660 | 2660 | AUAUAUUCUGAGUUUUGUG | 9014 | CACAAAACUCAGAAUAUAU |

The siRNAs in subset I have the following characteristics:
Cross-reactivity: With 19mer in human GPAM mRNA, with 17mer or 19mer in NHP GPAM mRNA, and in some cases 17mer or 19mer in mouse GPAM.
Specificity category: For human and NHP only cross-reactive siRNAs: human AS1 or better, human SS3 or better. For human, NHP and mouse cross-reactive siRNAs, human AS2 or better, human AS3 or better.
miRNA seeds: For human and NHP only cross-reactive siRNAs: AS and SS: seed region not in any human siRNA, seed region not conserved in human, mouse, and rat and not present in >3 species. For human, NHP and mouse cross-reactive siRNAs: seed region not in any human siRNA, seed region not conserved in human, mouse, and rat and not present in >1 species.
SNPs: siRNA target sites do not harbor SNPs with a MAF≥1% (pos. 2-18)

The siRNAs targeting human GPAM can also be selected using at least Selection Set C criteria, and including cross-reactivity with the NHP cynomolgus GPAM. Selection of these siRNAs yields Subset J. Subset J includes 24 siRNAs whose base sequences are shown in Table 11B.

TABLE 11B
Subset J siRNAs

| | Sense | | Antisense | |
|---|---|---|---|---|
| | strand | | strand | |
| siRNA | SEQ ID | Sense strand | SEQ ID | Antisense strand |
| Name | NO: | sequence (5′-3′) | NO: | sequence (5′-3′) |
| siRNA 114 | 114 | AAACUGAUAGCUGAGUCCU | 6468 | AGGACUCAGCUAUCAGUUU |
| siRNA 194 | 194 | UUGUGACAUGGAUGAAUCU | 6548 | AGAUUCAUCCAUGUCACAA |
| siRNA 260 | 260 | AGAAUACAGUGUUGGUCGA | 6614 | UCGACCAACACUGUAUUCU |
| siRNA 262 | 262 | AAUACAGUGUUGGUCGAUG | 6616 | CAUCGACCAACACUGUAUU |
| siRNA 263 | 263 | AUACAGUGUUGGUCGAUGU | 6617 | ACAUCGACCAACACUGUAU |
| siRNA 264 | 264 | UACAGUGUUGGUCGAUGUA | 6618 | UACAUCGACCAACACUGUA |
| siRNA 278 | 278 | AUGUAAGCACACAAGUGAG | 6632 | CUCACUUGUGUGCUUACAU |
| siRNA 432 | 432 | UUUUUCAACCCCAGUAUCC | 6786 | GGAUACUGGGGUUGAAAAA |
| siRNA 537 | 537 | CUUUUUAUUCAAGAGCGAG | 6891 | CUCGCUCUUGAAUAAAAAG |
| siRNA 542 | 542 | UAUUCAAGAGCGAGAUGUG | 6896 | CACAUCUCGCUCUUGAAUA |
| siRNA 956 | 956 | CAUUGCUUCAGGCAAUAAU | 7310 | AUUAUUGCCUGAAGCAAUG |
| siRNA 1105 | 1105 | AAUUACUUCGACAGCAGCA | 7459 | UGCUGCUGUCGAAGUAAUU |
| siRNA 1106 | 1106 | AUUACUUCGACAGCAGCAA | 7460 | UUGCUGCUGUCGAAGUAAU |
| siRNA 1191 | 1191 | CUUUUGUCAGUUGUGGUAG | 7545 | CUACCACAACUGACAAAAG |
| siRNA 1241 | 1241 | CUUGAUAAUACCUGUUGGA | 7595 | UCCAACAGGUAUUAUCAAG |
| siRNA 1243 | 1243 | UGAUAAUACCUGUUGGAAU | 7597 | AUUCCAACAGGUAUUAUCA |
| siRNA 1346 | 1346 | AAGAGGUGUUAUUAGAAUG | 7700 | CAUUCUAAUAACACCUCUU |
| siRNA 1363 | 1363 | UGUUACGAAAAAACUAUGG | 7717 | CCAUAGUUUUUUCGUAACA |
| siRNA 1600 | 1600 | AUCUGGCUGAGCAUAUUCU | 7954 | AGAAUAUGCUCAGCCAGAU |
| siRNA 1617 | 1617 | CUAUUCACUGCUAGCAAGU | 7971 | ACUUGCUAGCAGUGAAUAG |
| siRNA 1795 | 1795 | UAGUAAUGCAUGCCAUACA | 8149 | UGUAUGGCAUGCAUUACUA |
| siRNA 1920 | 1920 | AAUGGGGUACUUCAUGUCU | 8274 | AGACAUGAAGUACCCCAUU |
| siRNA 2141 | 2141 | AGGAAAGUUUAUCCAGUAU | 8495 | AUACUGGAUAAACUUUCCU |
| siRNA 2453 | 2453 | UCUGCAAAAGUUGCACAAA | 8807 | UUUGUGCAACUUUUGCAGA |

The siRNAs in subset J have the following characteristics:
Cross-reactivity: With 19mer in human GPAM mRNA, with 17mer or 19mer in NHP GPAM mRNA.
Specificity category: human AS2 or better, human SS3 or better. NHP AS2 or better, NHP SS3 or better
miRNA seeds: For human and NHP only cross-reactive siRNAs: AS strand: seed region not in any human siRNA, seed region not conserved in human, mouse, and rat and not present in >4 species. SS strand: seed region not conserved in human, mouse, and rat and not present in >4 species.
SNPs: siRNA target sites do not harbor SNPs with a MAF>10% (pos. 2-18)

### Example 3: Chemically Modified GPAM siRNAs

Any set of modifications, or any modification pattern may be used. For example, The siRNAs targeting GPAM can be synthesized with chemical modifications with the sense strand having modification pattern 1S and antisense strand having modification pattern 1AS. In some embodiments, the siRNAs targeting GPAM can also be synthesized with chemical modifications with the sense strand having modification pattern 2S and antisense strand having modification pattern 3AS. In some embodiments, the modifications included in Table 10 or Table 11 are used. In addition, adenosine can be placed at position 19 in the sense strand and uridine at position 1 in the antisense strand.

### Example 4: SiRNA-Mediated Knockdown of GPAM in Human Hepatocyte Cells

siRNAs targeted to the GPAM mRNA that downregulate levels of GPAM mRNA may lead to subsequent decrease of lysophosphatidic acid (LPA), a metabolite produced by the GPAM gene product, when administered to cultured hepatocyte cells.

On Day 0, the hepatocyte cells are to be seeded at 150,000 cells/mL into a Falcon 24-well tissue culture plate (ThermoFisher Cat. No. 353047) at 0.5 mL per well.

On Day 1, the GPAM siRNA and negative control siRNA master mixes are prepared. The GPAM siRNA master mix contains 350 uL of Opti-MEM (ThermoFisher Cat. No. 4427037-s1288 Lot No. AS02B02D) and 3.5 ul of a mixture of the two GPAM siRNAs (10 uM stock). The negative control siRNA master mix contains 350 uL of Opti-MEM and 3.5 ul of negative control siRNA (ThermoFisher Cat. No. 4390843, 10 uM stock). Next, 3 uL of TransIT-X2 (Minis Cat. No. MIR6000) is added to each master mix. The mixes are incubated for 15 minutes to allow transfection complexes to form, then 51 ul of the appropriate master mix+TransIT-X2 is added to duplicate wells of hepatocyte cells with a final siRNA concentration of 10 nM.

On Day 3, 48 hours post transfection, cells are lysed using the Cells-to-Ct kit according to the manufacturer's protocol (ThermoFisher Cat. No. 4399002). In brief, cells are washed with 50 ul using cold IX PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR was performed in triplicate using TaqMan Gene Expression Assays (Applied Biosystems FAM/GPAM using a BioRad iCycler).

A decrease in GPAM mRNA expression in the hepatocyte cells is expected after transfection with the GPAM siRNAs compared to GPAM mRNA levels in hepatocyte cells transfected with the non-specific control siRNA 48 hours after transfection. There is an expected decrease in the amount of LPA in wells containing hepatocytes cells transfected with the GPAM siRNAs relative to the amount of LPA in wells containing hepatocyte cells transfected with a non-specific control siRNA 48 hours after transfection. These results show that the GPAM siRNAs elicit knockdown of GPAM mRNA in hepatocyte cells and that the decrease in GPAM expression is correlated with a decrease in LPA production.

The GPAM siRNAs showing the greatest degree of knockdown of GPAM mRNA at 10 nM will be tested in a second screen for activity at 1 nM concentration using the transfection procedures as described above.

### Example 5: SiRNA-Mediated Knockdown of GPAM in Human Adipocyte Cells

Adipose tissue is a major site of endogenous triglyceride synthesis. siRNAs targeted to the GPAM mRNA that downregulate levels of GPAM mRNA may lead to subsequent decrease of lysophosphatidic acid (LPA), a metabolite produced by the GPAM gene product, when administered to cultured adipocyte cells. A decrease in LPA production may lead to a subsequent decrease in the levels of other components of the glycerol phosphate pathway for de novo triacylglycerol synthesis. Namely, phosphatidic acid (PA), phosphatidylserine (DAG), and triacylglycerol (TAG) may be decreased after siRNA-mediated knockdown of GPAM in human adipocyte cells.

On Day 0, the differentiated adipocyte cells (Cell Applications, Inc.) are to be seeded at 150,000 cells/mL into a Falcon 24-well tissue culture plate (ThermoFisher Cat. No. 353047) at 0.5 mL per well and cultured in adipocyte maintenance medium according to manufacturer's instructions.

On Day 1, the GPAM siRNA and negative control siRNA master mixes are prepared. The GPAM siRNA master mix contains 350 uL of Opti-MEM (ThermoFisher Cat. No. 4427037-s1288 Lot No. AS02B02D) and 3.5 ul of a mixture of the two GPAM siRNAs (10 uM stock). The negative control siRNA master mix contains 350 uL of Opti-MEM and 3.5 ul of negative control siRNA (ThermoFisher Cat. No. 4390843, 10 uM stock). Next, 3 uL of TransIT-X2 (Minis Cat. No. MIR6000) is added to each master mix. The mixes are incubated for 15 minutes to allow transfection complexes to form, then 51 ul of the appropriate master mix+TransIT-X2 is added to duplicate wells of differentiated adipocyte cells with a final siRNA concentration of 10 nM.

On Day 3, 48 hours post transfection, cells are lysed using the Cells-to-Ct kit according to the manufacturer's protocol (ThermoFisher Cat. No. 4399002). In brief, cells are washed with 50 ul using cold IX PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR was performed in triplicate using TaqMan Gene Expression Assays (Applied Biosystems FAM/GPAM using a BioRad iCycler).

A decrease in GPAM mRNA expression in the adipocyte cells is expected after transfection with the GPAM siRNAs compared to GPAM mRNA levels in adipocyte cells transfected with the non-specific control siRNA 48 hours after transfection. There is an expected decrease in the amount of LPA in wells containing adipocyte cells transfected with the GPAM siRNAs relative to the amount of LPA in wells containing adipocyte cells transfected with a non-specific control siRNA 48 hours after transfection. These results show that the GPAM siRNAs elicit knockdown of GPAM mRNA in adipocyte cells and that the decrease in GPAM expression is correlated with a decrease in LPA production. PA, DAG, and TAG levels are measured in wells containing adipocyte cells transfected with a GPAM siRNA and with a non-specific control siRNA 48 hours after transfection if there was compensation for reduced GPAM activity at various steps of the triacylglycerol synthesis pathway. These results show that the GPAM siRNAs elicit knockdown of GPAM mRNA in adipocyte cells and that the decrease in GPAM expression is correlated with a decrease in LPA production, PA production, DAG production, and TAG production.

The GPAM siRNAs showing the greatest degree of knockdown of GPAM mRNA at 10 nM will be tested in a second screen for activity at 1 nM concentration using the transfection procedures as described above.

### Example 6: ASO-Mediated Knockdown of GPAM in Human Hepatocyte Cells

ASOs targeted to the GPAM mRNA that downregulate levels of GPAM mRNA leading to subsequent decrease of LPA, a metabolite produced by the GPAM gene product, when administered to cultured hepatocyte cells.

On Day 0, the hepatocyte cells are seeded at 150,000 cells/mL into a Falcon 24-well tissue culture plate (Cat. No. 353047) at 0.5 mL per well.

On Day 1, the GPAM ASO and negative control ASO master mixes are prepared. The GPAM ASO master mix contains 350 uL of Opti-MEM (ThermoFisher Cat. No. 4427037-s1288 Lot No. AS02B02D) and 3.5 ul of a mixture of the two GPAM ASOs (10 uM stock). The negative control ASO master mix contains 350 uL of Opti-MEM and 3.5 ul of negative control ASO (ThermoFisher Cat. No. 4390843, 10 uM stock). Next, 3 uL of TransIT-X2 (Mimus Cat. No. MIR6000) is added to each master mix. The mixes are incubated for 15 minutes to allow transfection complexes to form, then 51 ul of the appropriate master mix+TransIT-X2 is added to duplicate wells of hepatocyte cells with a final ASO concentration of 10 nM.

On Day 3, 48 hours post transfection, cells are lysed using the Cells-to-Ct kit according to the manufacturer's protocol (ThermoFisher Cat. No. 4399002). In brief, cells are washed with 50 ul using cold IX PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR was performed in triplicate using TaqMan Gene Expression Assays (Applied Biosystems FAM/GPAM using a BioRad iCycler).

A decrease in GPAM mRNA expression in the hepatocyte cells is expected after transfection with the GPAM ASOs compared to GPAM mRNA levels in hepatocyte cells transfected with the non-specific control ASO 48 hours after transfection. There is an expected decrease in the amount of LPA in wells containing hepatocytes cells transfected with the GPAM ASOs relative to the amount of LPA in wells containing hepatocyte cells transfected with a non-specific control ASO 48 hours after transfection. These results show that the GPAM ASOs elicit knockdown of GPAM mRNA in hepatocyte cells and that the decrease in GPAM expression is correlated with a decrease in LPA production.

### Example 7: Inhibition of GPAM in a Mouse Model for Fatty Liver Disease Using Modified GPAM siRNAs and ASOs

A murine model of fatty liver disease is used to evaluate the effect of siRNA or ASO inhibition of GPAM. In the murine model, fatty liver disease is induced by feeding mice a Western Diet (WD) containing 21.1% fat, 41% Sucrose, and 1.25% Cholesterol by weight (Teklad diets, TD. 120528) and a high sugar solution (23.1 g/L d-fructose (Sigma-Aldrich, G8270) and 18.9 g/L d-glucose (Sigma-Aldrich, F0127)) for 12 weeks. At 4-week-old C57BL/6J mice are fed a Western Diet instead of regular chow for 12 weeks.

Briefly, mice are divided into five groups: Group 1—a fatty liver disease group treated with non-targeting control siRNA, Group 2—a fatty liver disease group treated with non-targeting control ASO, Group 3—a fatty liver disease group treated with GPAM siRNA 1, Group 4—a fatty liver disease group treated with GPAM ASO1, Group 5—control mice on a normal chow diet. Each group contains eight mice (4 males, 4 females).

At weeks 12 weeks of Western Diet, blood samples are collected from each group prior to the first treatment.

Administration of siRNA or ASO is achieved with a 200 ul subcutaneous injection of siRNA or ASO resuspended in PBS at concentration of 10 uM. On Study Day 0, Group 1 mice are injected subcutaneously with non-targeting control siRNA, Group 2 mice are injected subcutaneously with non-targeting control ASO, Group 3 mice are injected subcutaneously with siRNA1 targeting mouse GPAM, Group 4 mice are injected subcutaneously with ASO1 targeting mouse GPAM, and Group 5 mice are injected subcutaneously with vehicle. Every other week thereafter starting on Day 14 the animals from each group are dosed as on Day 0 for a total of 5 injections.

Weekly blood draws are taken and serum and plasma isolated. ALT, AST, ALP bilirubin, total cholesterol, HDL cholesterol and triglyceride levels are measured using VITROS 5,1 FS (Ortho Clinical Diagnostics). Non-fasting plasma insulin is measured with the Ultrasensitive Mouse Insulin ELISA kit (Crystal Chem, 90080) according to the manufacturer's instructions. Non-fasting blood glucose is assayed with the One Touch Ultra (Life Scan). HOMA IR and QUICKI are calculated. Blood ketones, including 3-hydroxybutyrate, acetoacetate and acetone are measured using EnzyChrom Ketone Body Assay Kit (BioAssay Systems, EKBD-100).

At the end of 12 weeks of Western Diet and siRNA/ASO treatment, mice are sacrificed by cervical dislocation following an intraperitoneal injection of 0.3 ml Nembutal (5 mg/ml). Terminal blood draw is collected via cardiac puncture and final ALT, AST, ALP bilirubin, total cholesterol, HDL cholesterol and triglyceride levels are measured along with non-fasting plasma insulin and glucose and blood ketones. Livers and adipose tissue are removed and divided into three sections each; one section placed in RNAlater for mRNA isolation, one section flash-frozen for protein isolation, one section fixed in formalin and then paraffin-embedded.

mRNA is isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR was performed in triplicate using TaqMan Gene Expression Assays (Applied Biosystems FAM/GPAM using a BioRad iCycler). A decrease in GPAM mRNA expression in the liver and adipose tissue from mice is dosed with the GPAM siRNAs and ASOs compared to GPAM mRNA levels in the liver and adipose tissue from mice is dosed with the non-specific control siRNA and ASO. There is an expected decrease in the amount of SDF-1 in the liver and adipose tissue from mice that receive the GPAM siRNAs and ASOs compared to the amount of SDF-1 in the liver and adipose tissue from mice that receive the non-specific control siRNA or ASO. These results show that the GPAM siRNAs and ASOs elicit knockdown of GPAM mRNA in liver and adipose tissue and that the decrease in GPAM expression is correlated with a decrease in SDF-1 production. There is an expected decrease in blood ALT, AST, ALP, bilirubin and total cholesterol, and an expected increase in blood ketones in mice that receive the GPAM siRNA or ASO compared to the blood ALT, AST, ALP, bilirubin, total cholesterol and ketones in mice that receive the non-specific control. These results show that the GPAM siRNA or ASO elicit knockdown of GPAM mRNA in liver and adipose tissue and that the decrease in GPAM expression is correlated with a decrease in blood ALT, AST, ALP, bilirubin and total cholesterol, and an increase in blood ketones.

Formalin-fixed, paraffin-embedded liver sections are stained with hematoxylin and eosin (H&E) for assessment of liver histology, with Sirius Red (Sigma, 365548-5G)/Fast Green (Sigma, F258) for assessment of fibrosis, and with periodic acid-Schiff (PAS) for assessment of glycogen accumulation. NAFLD Activity Score (NAS) and fibrosis stage are evaluated by an expert pathologist according to the NASH CRN scoring system. The histological scoring is performed blinded, with no knowledge by the pathologist of the treatment(s) received. These results show that the GPAM siRNAs and ASOs elicit knockdown of GPAM mRNA in liver tissue and that the decrease in GPAM expression is correlated with a decrease in NAS and NASH CRN.

### Example 8: Oligonucleotide Synthesis

Oligonucleotides such as siRNAs may be synthesized according to phosphoramidite technology on a solid phase. For example, a K&A oligonucleotide synthesizer may be used. Syntheses may be performed on a solid support made of controlled pore glass (CPG, 500 Å or 600 Å, obtained from AM Chemicals, Oceanside, CA, USA). All 2′-OMe and 2′-F phosphoramidites may be purchased from Hongene Biotech (Union City, CA, USA). All phosphoramidites may be dissolved in anhydrous acetonitrile (100 mM) and molecular sieves (3 Å) may be added. 5-Benzylthio-1H-tetrazole (BTT, 250 mM in acetonitrile) or 5-Ethylthio-1H-tetrazole (ETT, 250 mM in acetonitrile) may be used as activator solution. Coupling times may be 9-18 min (e.g. with a GalNAc such as ETL17), 6 min (e.g. with 2′OMe and 2′F). In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile may be employed.

After solid phase synthesis, the dried solid support may be treated with a 1:1 volume solution of 40 wt. % methylamine in water and 28% ammonium hydroxide solution (Aldrich) for two hours at 30° C. The solution may be evaporated and the solid residue may be reconstituted in water and purified by anionic exchange HPLC using a TKSgel SuperQ-5PW 13u column. Buffer A may be 20 mM Tris, 5 mM EDTA, pH 9.0 and contained 20% Acetonitrile and buffer B may be the same as buffer A with the addition of 1 M sodium chloride. UV traces at 260 nm may be recorded. Appropriate fractions may be pooled then desalted using Sephadex G-25 medium.

Equimolar amounts of sense and antisense strand may be combined to prepare a duplex. The duplex solution may be prepared in 0.1×PBS (Phosphate-Buffered Saline, 1×, Gibco). The duplex solution may be annealed at 95° C. for 5 min, and cooled to room temperature slowly. Duplex concentration may be determined by measuring the solution absorbance on a UV-Vis spectrometer at 260 nm in 0.1×PBS. For some experiments, a conversion factor may be calculated from an experimentally determined extinction coefficient.

### Example 9: GalNAc Ligand for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. Reagents for GalNAc conjugation to oligonucleotides are shown in Table 12.

TABLE 12
GalNAc Conjugation Reagents

| Type of | |
|---|---|
| conjugation | Structure |
| Solid phase 3′ attachment where squiggly line is rest of oligonucleotide chain and right-most OH is where attachment to solid phase is. | 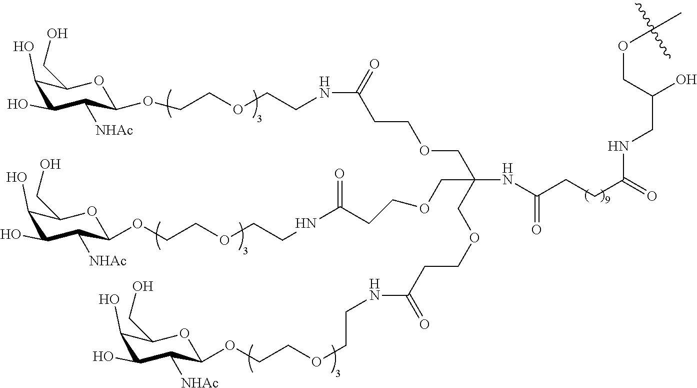 |
| | This GalNAc ligand may be referred to as “GalNAc23” or “GalNAc#23.” |
| Solid phase 5′ attachment phosphoramidite | 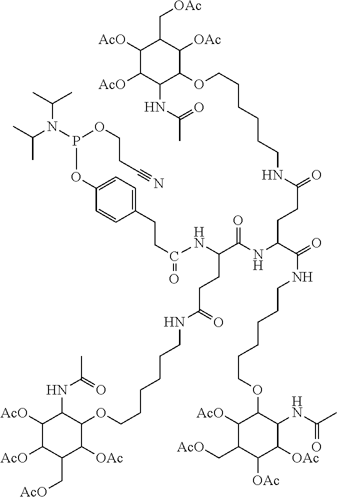 [SMILES: CC(=O)NC1C(OCCCCCCNC(=O)CCC(NC(=O)CCc2ccc(OP(OCCC#N)N(C(C)C)C(C)C)cc2)C(=O)NC(CCC(=O)NCCCCCCOC2C(NC(C)=O)C(OC(C)=O)C(OC(C)=O)C(COC(C)=O)C2OC(C)=O)C(=O)NCCCCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solid phase 5' attachment Phosphoramidite | 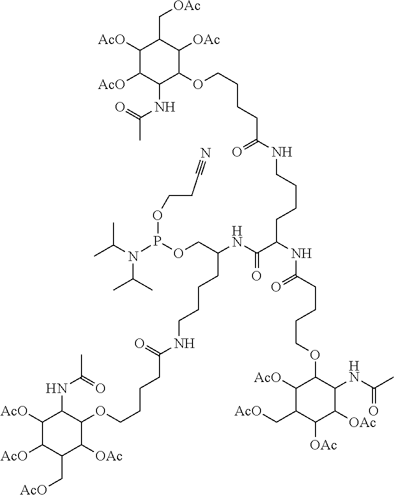 [SMILES: CC(=O)NC1C(OCCCCC(=O)NCCCCC(COP(OCCC#N)N(C(C)C)C(C)C)NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solution phase Carboxylic acid for amide coupling anywhere on oligonucleotide | 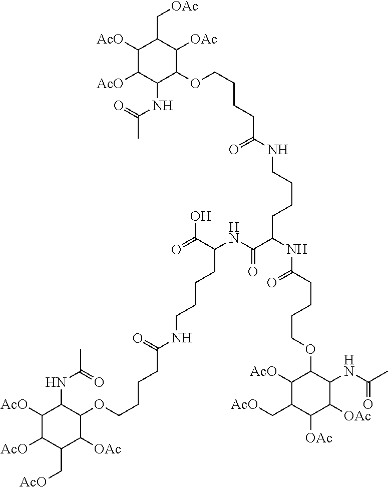 [SMILES: CC(=O)NC1C(OCCCCC(=O)NCCCCC(NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(=O)O)C(OC(C)=O)C(COC(C)=O)C(OC(C)=O)C1OC(C)=O] |
| | Where Ac is an acetyl group or other hydroxyl protecting group that can be removed |
| | under basic, acid or reducing conditions. |

In solution phase conjugation, the oligonucleotide sequence—including a reactive conjugation site—is formed on the resin. The oligonucleotide is then removed from the resin and GalNAc is conjugated to the reactive site.

The carboxy GalNAc derivatives may be coupled to amino-modified oligonucleotides. The peptide coupling conditions are known to the skilled in the art using a carbodiimide coupling agent like DCC (N,N′-Dicyclohexylcarbodiimide), EDC (N-(3-dimethylaminopropyl)-N′-ethylcarbodiimide) or EDC·HCl (N-(3-dimethylaminopropyl)-N′-ethylcarbodiimide hydrochloride and an additive like HOBt (1-hydroxybenztriazole), HOSu (N-hydroxysuccinimide), TBTU (N,N,N′,N′-Tetramethyl-O-(benzotriazol-1-yl)uronium tetrafluoroborate, HBTU (2-(1H-benzotriazol-1-yl)-1,1,3,3-tetramethyluronium hexafluorophosphate) or HOAt (1-Hydroxy-7-azabenzotriazole and common combinations thereof such as TBTU/HOBt or HBTU/HOAt to form activated amine-reactive esters.

Amine groups may be incorporated into oligonucleotides using a number of known, commercially available reagents at the 5′ terminus, 3′ terminus or anywhere in between.

Non-limiting examples of reagents for oligonucleotide synthesis to incorporate an amino group include:
5′ attachment:
6-(4-Monomethoxytritylamino)hexyl-(2-cyanoethyl)-(N,N-diisopropyl)-phosphoramidite CAS Number: 114616-27-2
5′-Amino-Modifier TEG CE-Phosphoramidite
10-(O-trifluoroacetamido-N-ethyl)-triethyleneglycol-1-[(2-cyanoethyl)-(N,N-diisopropyl)]-phosphoramidite
3′ attachment:
3′-Amino-Modifier Serinol CPG
3-Dimethoxytrityloxy-2-(3-(fluorenylmethoxycarbonylamino)propanamido)propyl-1-O-succinyl-long chain alkylamino-CPG (where CPG stands for controlled-pore glass and is the solid support)
Amino-Modifier Serinol Phosphoramidite
3-Dimethoxytrityloxy-2-(3-(fluorenylmethoxycarbonylamino)propanamido)propyl-1-O-(2-cyanoethyl)-(N,N-diisopropyl)-phosphoramidite

### Internal (Base Modified):

Amino-Modifier C₆ dT
5′-Dimethoxytrityl-5-[N-(trifluoroacetylaminohexyl)-3-acrylimido]-2′-deoxyUridine, 3′-[(2-cyanoethyl)-(N,N-diisopropyl)]-phosphoramidite. CAS Number: 178925-21-8

Solution phase conjugations may occur after oligonucleotide synthesis via reactions between non-nucleosidic nucleophilic functional groups that are attached to the oligonucleotide and electrophilic GalNAc reagents. Examples of nucleophilic groups include amines and thiols, and examples of electrophilic reagents include activated esters (e.g. N-hydroxysuccinimide, pentafluorophenyl) and maleimides.

### Example 10: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. A non-limiting example of a phosphoramidite reagent for GalNAc conjugation to a 5′ end oligonucleotide is shown in Table 13.

TABLE 13
GalNAc Conjugation Reagent

| Type of | |
|---|---|
| con- | |
| jugation | Structure |
| Solid phase 5′ attach- ment phos- phora- midite | 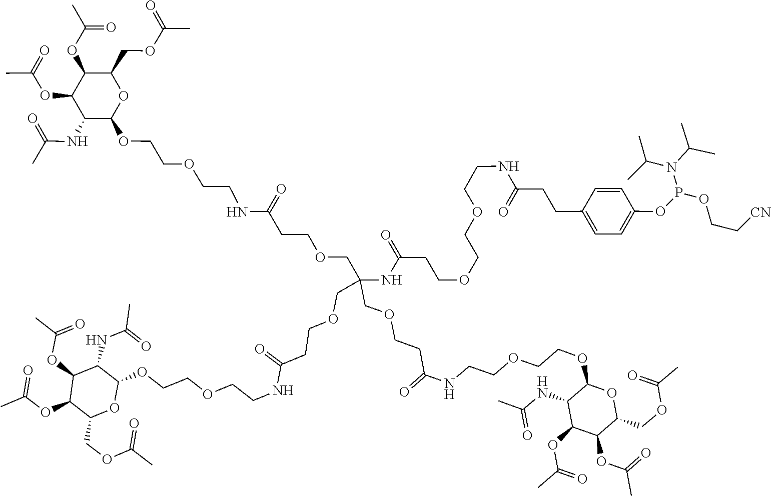 [SMILES: CC(=O)N[C@@H]1[C@H](OCCOCCNC(=O)CCOCC(COCCC(=O)NCCOCCO[C@H]2O[C@H](COC(C)=O)[C@@H](OC(C)=O)[C@@H](OC(C)=O)[C@@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)N(C(C)C)C(C)C)cc2)O[C@H](COC(C)=O)[C@@H](OC(C)=O)[C@H]1OC(C)=O] |

The following includes examples of synthesis reactions used to create a GalNAc moiety:

### Scheme for the Preparation of NAcegal-Linker-TMSOTf

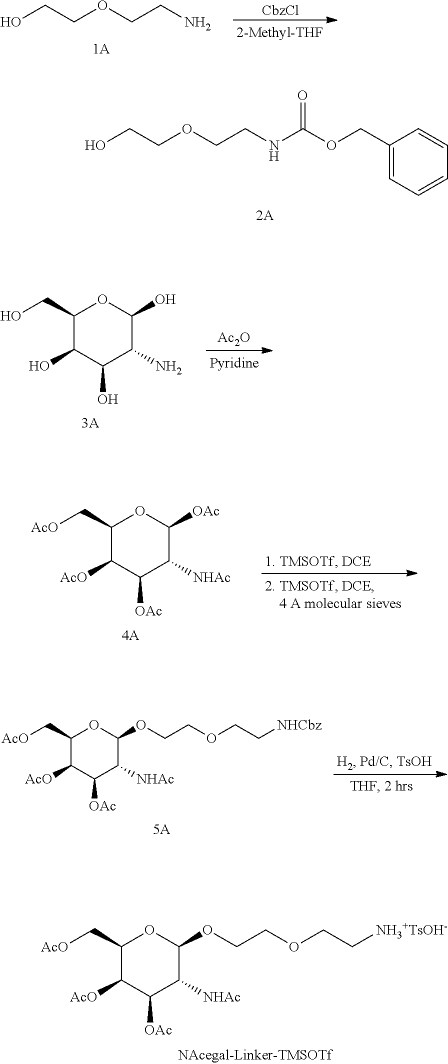

### General Procedure for Preparation of Compound 2A

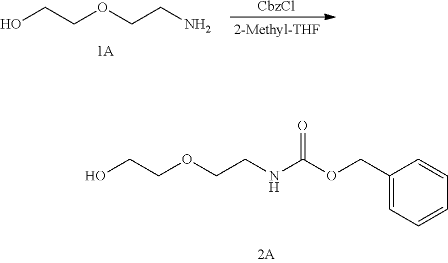

To a solution of Compound 1A (500 g, 4.76 mol, 476 mL) in 2-Methyl-THF (2.00 L) is added CbzCl (406 g, 2 38 mol, 338 mL) in 2-Methyl-THF (750 mL) dropwise at 0° C. The mixture is stirred at 25° C. for 2 hrs under N₂ atmosphere. TLC (DCM:MeOH=20:1, PMA) may indicate CbzCl is consumed completely and one new spot (R_{f}=0.43) formed. The reaction mixture is added HCl/EtOAc (1 N, 180 mL) and stirred for 30 mins, white solid is removed by filtration through celite, the filtrate is concentrated under vacuum to give Compound 2A (540 g, 2.26 mol, 47.5% yield) as a pale yellow oil and used into the next step without further purification. ¹H NMR: δ 7.28-7.41 (m, 5H), 5.55 (br s, 1H), 5.01-5.22 (m, 2H), 3.63-3.80 (m, 2H), 3.46-3.59 (m, 4H), 3.29-3.44 (m, 2H), 2.83-3.02 (m, 1H).

### General Procedure for Preparation of Compound 4A

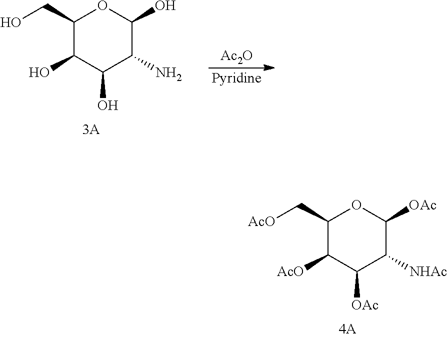 [SMILES: "Ac2O" ; N[C@@H]1[C@@H](O)[C@@H](O)[C@@H](CO)O[C@H]1O ; "Pyridine" ; CC(=O)N[C@H]1[C@H](OC(C)=O)O[C@H](COC(C)=O)[C@H](OC(C)=O)[C@@H]1OC(C)=O]

To a solution of Compound 3A (1.00 kg, 4.64 mol, HCl) in pyridine (5.00 L) is added acetyl acetate (4.73 kg, 46.4 mol, 4.34 L) dropwise at 0° C. under N₂ atmosphere. The mixture is stirred at 25° C. for 16 hrs under N₂ atmosphere. TLC (DCM:MeOH=20:1, PMA) indicated Compound 3A is consumed completely and two new spots (R_{f}=0.35) formed. The reaction mixture is added to cold water (30.0 L) and stirred at 0° C. for 0.5 hr, white solid formed, filtered and dried to give Compound 4A (1.55 kg, 3.98 mol, 85.8% yield) as a white solid and used in the next step without further purification. 1H NMR: 5 7.90 (d, J=9.29 Hz, 1H), 5.64 (d, J=8.78 Hz, 1H), 5.26 (d, J=3.01 Hz, 1H), 5.06 (dd, J=11.29, 3.26 Hz, 1H), 4.22 (t, J=6.15 Hz, 1H), 3.95-4.16 (m, 3H), 2.12 (s, 3H), 2.03 (s, 3H), 1.99 (s, 3H), 1.90 (s, 3H), 1.78 (s, 3H).

### General Procedure for Preparation of Compound 5A

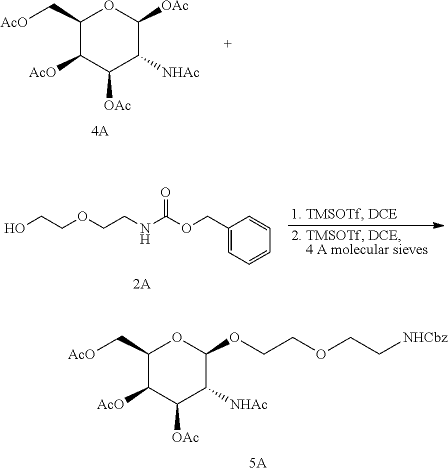

To a solution of Compound 4A (300 g, 771 mmol) in DCE (1.50 L) is added TMSOTf (257 g, 1.16 mol, 209 mL) and stirred for 2 hrs at 60° C., and then stirred for 1 hr at 25° C. Compound 2A (203 g, 848 mmol) is dissolved in DCE (1.50 L) and added 4 Å powder molecular sieves (150 g) stirring for 30 mins under N₂ atmosphere. Then the solution of Compound 4A in DCE is added dropwise to the mixture at 0° C. The mixture is stirred at 25° C. for 16 hrs under N₂ atmosphere. TLC (DCM:MeOH=25:1, PMA) indicated Compound 4A is consumed completely and new spot (R_{f}=0.24) formed. The reaction mixture is filtered and washed with sat. NaHCO₃ (2.00 L), water (2.00 L) and sat. brine (2.00 L). The organic layer is dried over anhydrous Na₂SO4, filtered and concentrated under reduced pressure to give a residue. The residue is triturated with 2-Me-THE/heptane (5/3, v/v, 1.80 L) for 2 hrs, filtered and dried to give Compound 5A (225 g, 389 mmol, 50.3% yield, 98.4% purity) as a white solid. ¹H NMR: δ 7.81 (d, J=9.29 Hz, 1H), 7.20-7.42 (m, 6H), 5.21 (d, J=3.26 Hz, 1H), 4.92-5.05 (m, 3H), 4.55 (d, J=8.28 Hz, 1H), 3.98-4.07 (m, 3H), 3.82-3.93 (m, 1H), 3.71-3.81 (m, 1H), 3.55-3.62 (m, 1H), 3.43-3.53 (m, 2H), 3.37-3.43 (m, 2H), 3.14 (q, J=5.77 Hz, 2H), 2.10 (s, 3H), 1.99 (s, 3H), 1.89 (s, 3H), 1.77 (s, 3H).

### General Procedure for Preparation of NAcegal-Linker-Tosylate Salt

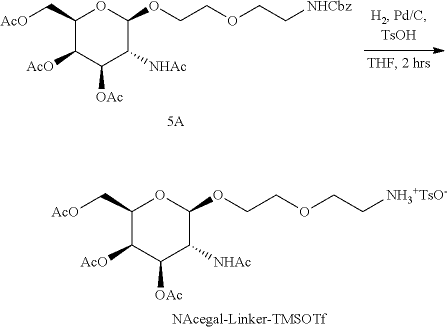

To a solution of Compound 5A (200 g, 352 mmol) in THF (1.0 L) is added dry Pd/C (15.0 g, 10% purity) and TsOH (60.6 g, 352 mmol) under N₂ atmosphere. The suspension is degassed under vacuum and purged with H₂ several times. The mixture is stirred at 25° C. for 3 hrs under H₂ (45 psi) atmosphere. TLC (DCM:MeOH=10:1, PMA) indicated Compound 5A is consumed completely and one new spot (R_{f}=0.04) is formed. The reaction mixture is filtered and concentrated (≤40° C.) under reduced pressure to give a residue. Diluted with anhydrous DCM (500 mL, dried overnight with 4 Å molecular sieves (dried at 300° C. for 12 hrs)) and concentrate to give a residue and run Karl Fisher (KF) to check for water content. This is repeated 3 times with anhydrous DCM (500 mL) dilutions and concentration to give NAcegal-Linker-TMSOTf (205 g, 95.8% yield, TsOH salt) as a foamy white solid. ¹H NMR: δ 7.91 (d, J=9.03 Hz, 1H), 7.53-7.86 (m, 2H), 7.49 (d, J=8.03 Hz, 2H), 7.13 (d, J=8.03 Hz, 2H), 5.22 (d, J=3.26 Hz, 1H), 4.98 (dd, J=11.29, 3.26 Hz, 1H), 4.57 (d, J=8.53 Hz, 1H), 3.99-4.05 (m, 3H), 3.87-3.94 (m, 1H), 3.79-3.85 (m, 1H), 3.51-3.62 (m, 5H), 2.96 (brt, J=5.14 Hz, 2H), 2.29 (s, 3H), 2.10 (s, 3H), 2.00 (s, 3H), 1.89 (s, 3H), 1.78 (s, 3H).

Scheme for the preparation of TRIS-PEG2-CBZ

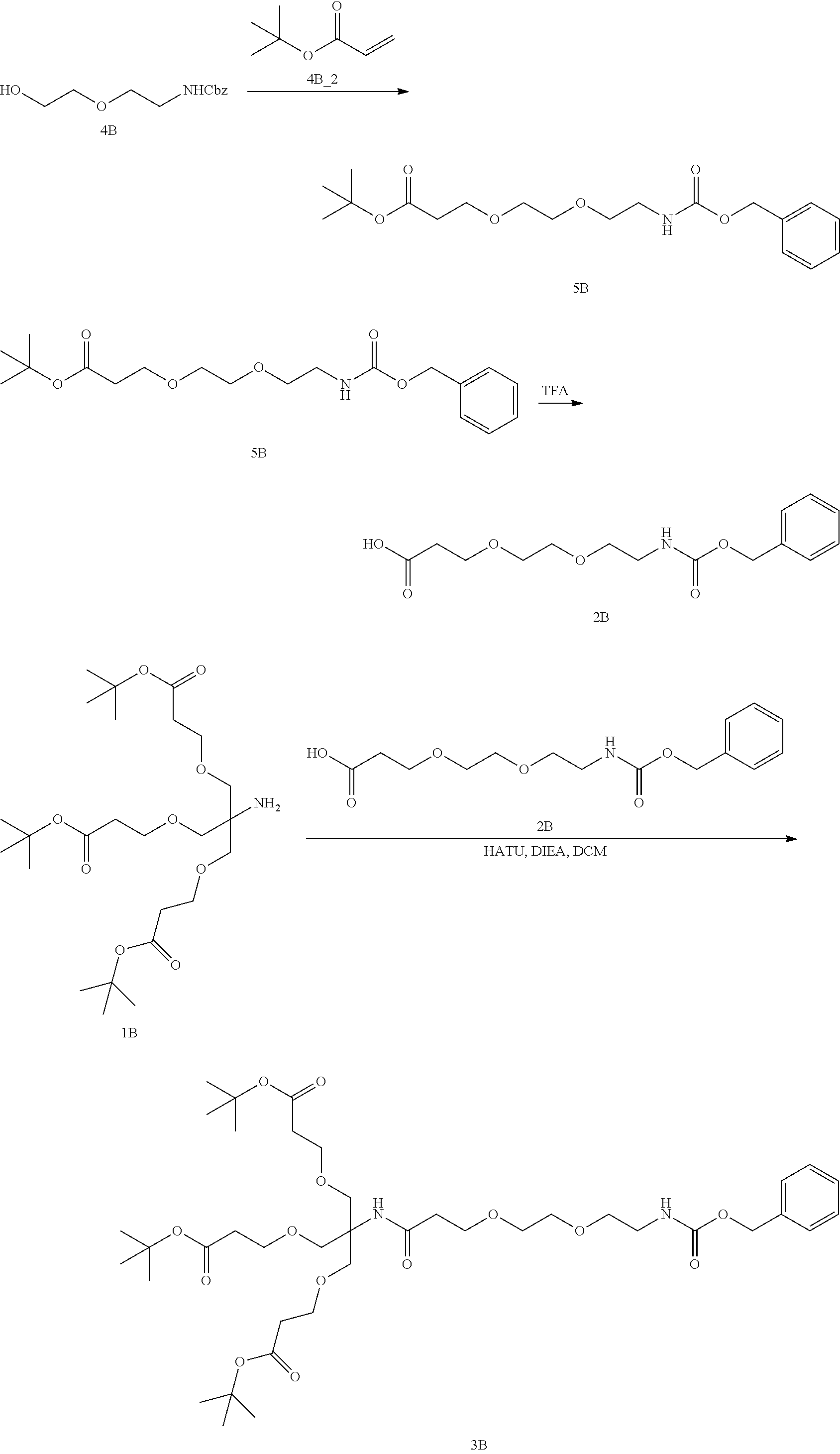

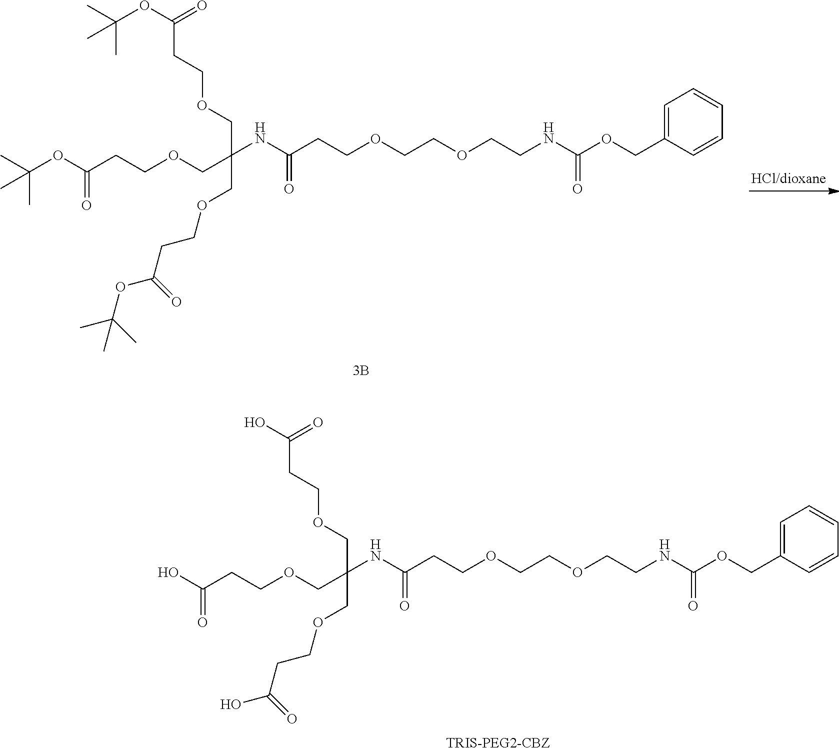 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

### General Procedure for Preparation of Compound 5B

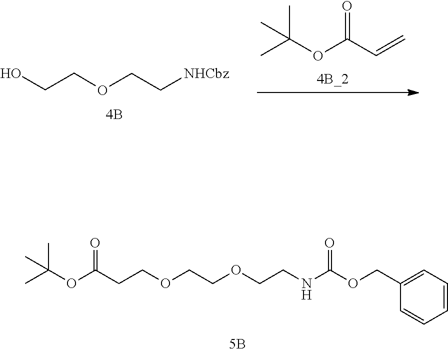

To a solution of Compound 4B (400 g, 1.67 mol, 1.00 eq) and NaOH (10 M, 16.7 mL, 0.10 eq) in THE (2.00 L) is added Compound 4B_2 (1.07 kg, 8.36 mol, 1.20 L, 5.00 eq), the mixture is stirred at 30° C. for 2 hrs. LCMS showed the desired MS is given. Five batches of solution are combined to one batch, then the mixture is diluted with water (6.00 L), extracted with ethyl acetate (3.00 L*3), the combined organic layer is washed with brine (3.00 L), dried over Na₂SO₄, filtered and concentrated under vacuum. The crude is purified by column chromatography (SiO₂, petroleum ether:ethyl acetate=100:1-10:1, R_{f}=0.5) to give Compound 5B (2.36 kg, 6.43 mol, 76.9% yield) as light yellow oil. HNMR: δ 7.31-7.36 (m, 5H), 5.38 (s, 1H), 5.11-5.16 (m, 2H), 3.75 (t, J=6.4 Hz), 3.54-3.62 (m, 6H), 3.39 (d, J=5.2 Hz), 2.61 (t, J=6.0 Hz).

### General Procedure for Preparation of 3-Oxo-1-Phenyl-2,7,10-Trioxa-4-Azatridecan-13-Oic Acid (Compound 2B Below)

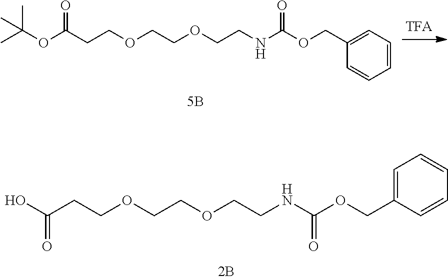 [SMILES: CC(C)(C)OC(=O)CCOCCOCCNC(=O)OCc1ccccc1 ; O=C(O)CCOCCOCCNC(=O)OCc1ccccc1]

To a solution of Compound 5B (741 g, 2.02 mol, 1.00 eq) in DCM (2.80 L) is added TFA (1.43 kg, 12.5 mol, 928 mL, 6.22 eq), the mixture is stirred at 25° C. for 3 hrs. LCMS showed the desired MS is given. The mixture is diluted with DCM (5.00 L), washed with water (3.00 L*3), brine (2.00 L), the combined organic layer is dried over Na₂SO₄, filtered and concentrated under vacuum to give Compound 2B (1800 g, crude) as light yellow oil. HNMR: δ 9.46 (s, 5H), 7.27-7.34 (m, 5H), 6.50-6.65 (m, 1H), 5.71 (s, 1H), 5.10-5.15 (m, 2H), 3.68-3.70 (m, 14H), 3.58-3.61 (m, 6H), 3.39 (s, 2H), 2.55 (s, 6H), 2.44 (s, 2H).

### General Procedure for Preparation of Compound 3B

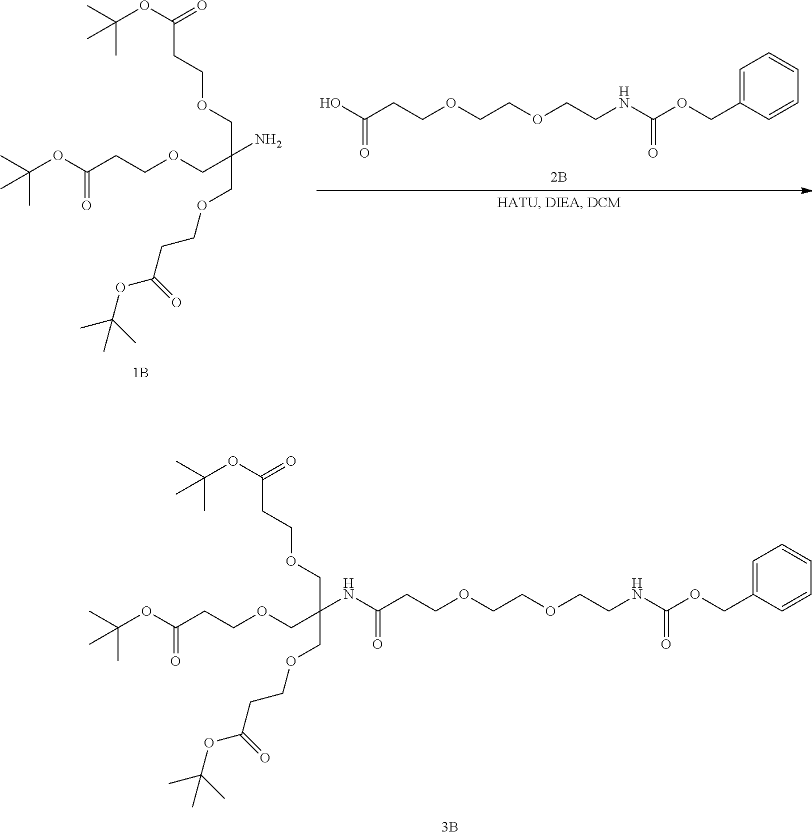 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

To a solution of Compound 2B (375 g, 999 mmol, 83.0% purity, 1.00 eq) in DCM (1.80 L) is added HATU (570 g, 1.50 mol, 1.50 eq) and DIEA (258 g, 2.00 mol, 348 mL, 2.00 eq) at 0° C., the mixture is stirred at 0° C. for 30 min, then Compound 1B (606 g, 1.20 mol, 1.20 eq) is added, the mixture is stirred at 25° C. for 1 hr. LCMS showed desired MS is given. The mixture is combined to one batch, then the mixture is diluted with DCM (5.00 L), washed with 1 N HCl aqueous solution (2.00 L*2), then the organic layer is washed with saturated Na₂CO₃ aqueous solution (2.00 L*2) and brine (2.00 L), the organic layer is dried over Na₂SO₄, filtered and concentrated under vacuum to give Compound 3B (3.88 kg, crude) as yellow oil.

### General Procedure for Preparation of TRIS-PEG2-CBZ.

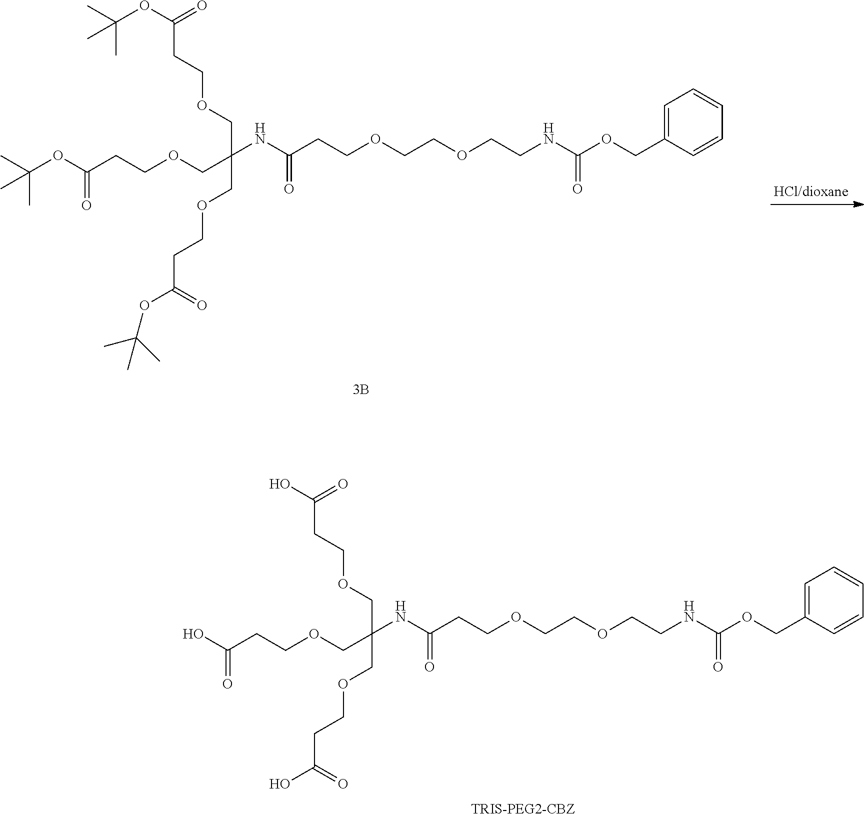 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

A solution of Compound 3B (775 g, 487 mmol, 50.3% purity, 1.00 eq) in HCl/dioxane (4 M, 2.91 L, 23.8 eq) is stirred at 25° C. for 2 hrs. LCMS showed the desired MS is given. The mixture is concentrated under vacuum to give a residue. Then the combined residue is diluted with DCM (5.00 L), adjusted to pH=8 with 2.5 M NaOH aqueous solution, and separated. The aqueous phase is extracted with DCM (3.00 L) again, then the aqueous solution is adjusted to pH=3 with 1 N HCl aqueous solution, then extracted with DCM (5.00 L*2), the combined organic layer is washed with brine (3.00 L), dried over Na₂SO₄, filtered and concentrated under vacuum. The crude is purified by column chromatography (SiO₂, DCM:MeOH=0:1-12:1, 0.10% HOAc, R_{f}=0.4). The residue is diluted with DCM (5.00 L), adjusted to pH=8 with 2.5 M NaOH aqueous solution, separated, the aqueous solution is extracted with DCM (3.00 L) again, then the aqueous solution is adjusted to pH=3 with 6 N HCl aqueous solution, extracted with DCM:MeOH=10:1 (5.00 L*2), the combined organic layer is washed with brine (2.00 L), dried over Na₂SO₄, filtered and concentrated under vacuum to give a residue. Then the residue is diluted with MeCN (5.00 L), concentrated under vacuum, repeat this procedure twice to remove water to give TRIS-PEG2-CBZ (1.25 kg, 1.91 mol, 78.1% yield, 95.8% purity) as light yellow oil. ¹HNMR: 400 MHz, MeOD, δ 7.30-7.35 (5H), 5.07 (s, 2H), 3.65-3.70 (m, 16H), 3.59 (s, 4H), 3.45 (t, J=5.6 Hz), 2.51 (t, J=6.0 Hz), 2.43 (t, 6.4 Hz).

### Scheme for the Preparation of TriNGal-TRIS-Peg2-Phosph 8c

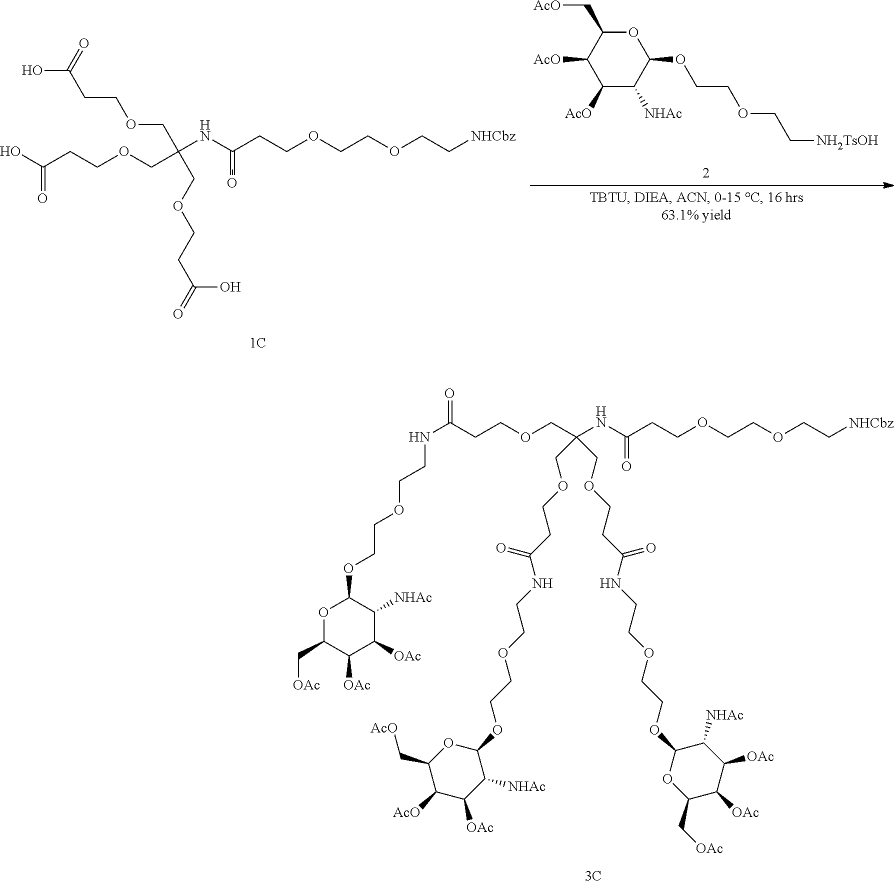

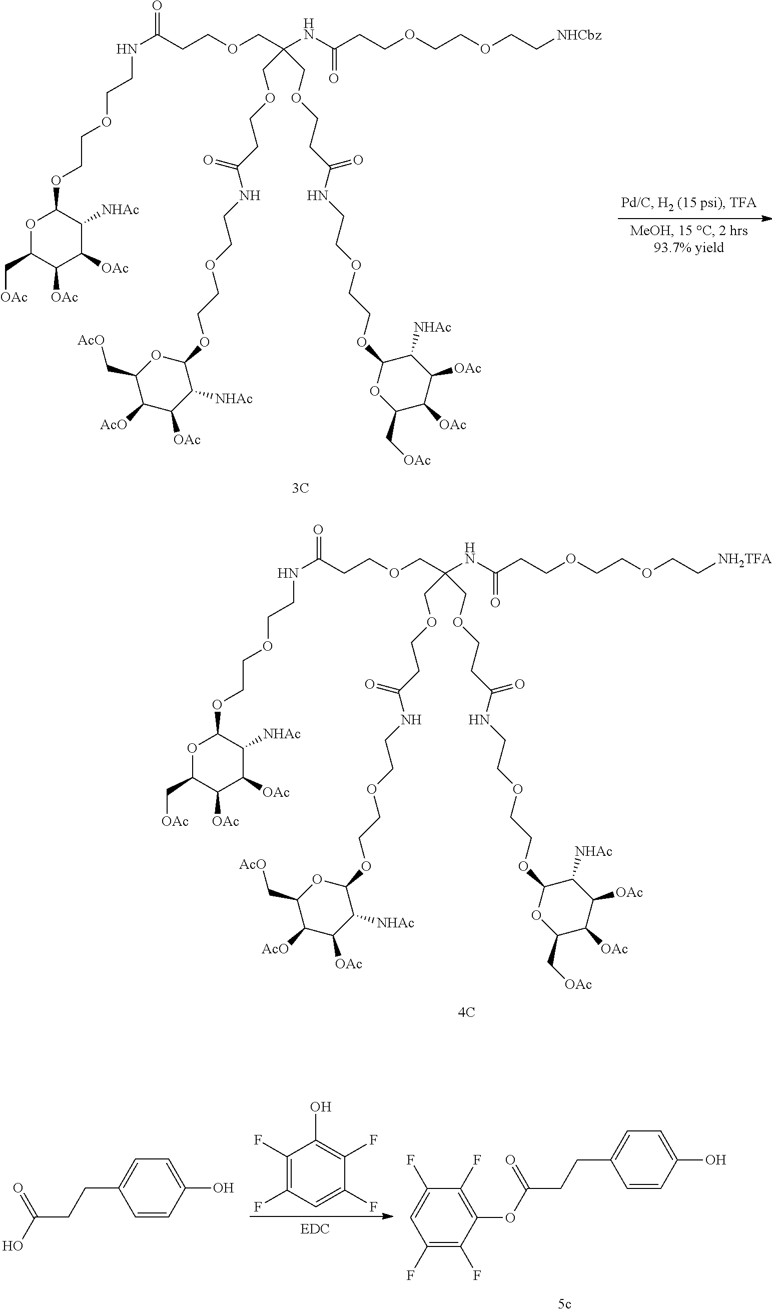

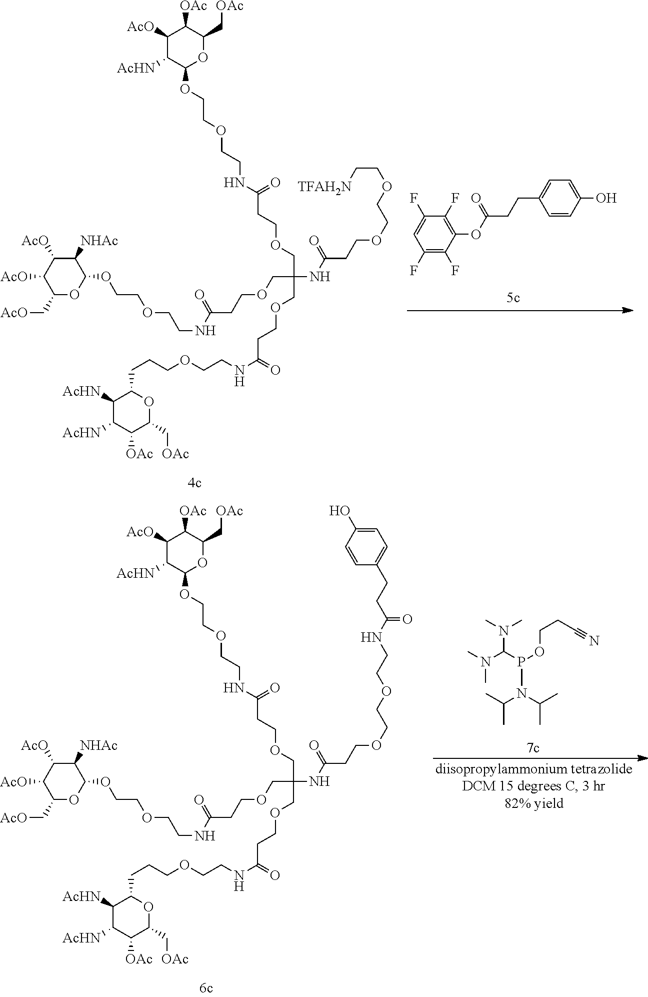

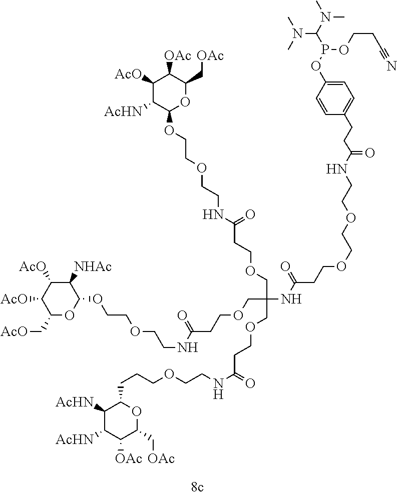 [SMILES: CC(=O)N[C@@H]1[C@@H](NC(C)=O)[C@H](CCCOCCNC(=O)CCOCC(COCCC(=O)NCCOCCO[C@@H]2O[C@H](COC(C)=O)[C@H](OC(C)=O)[C@H](OC(C)=O)[C@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)C(N(C)C)N(C)C)cc2)O[C@H](COC(C)=O)[C@@H]1OC(C)=O]

### TriGNal-TRIS-Peg2-Phosph 8c

### General Procedure for Preparation of Compound 3C

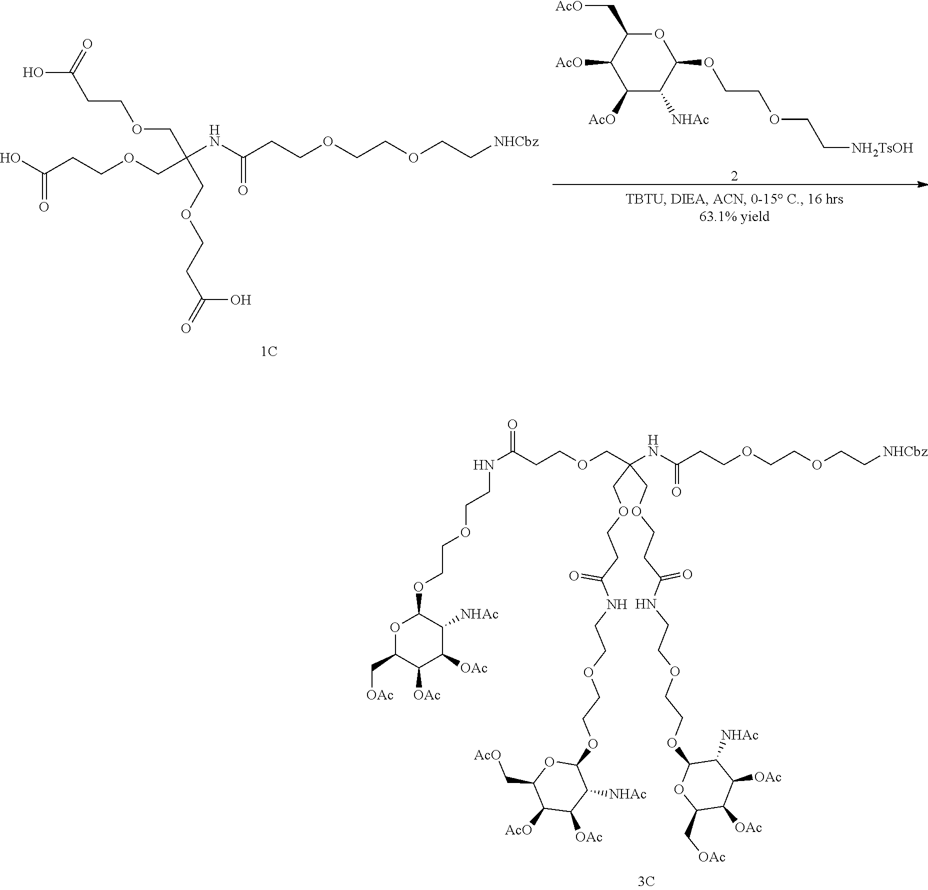

To a solution of Compound 1C (155 g, 245 mmol, 1.00 eq) in ACN (1500 mL) is added TBTU (260 g, 811 mmol, 3.30 eq), DIEA (209 g, 1.62 mol, 282 mL, 6.60 eq) and Compound 2C (492 g, 811 mmol, 3.30 eq, TsOH) at 0° C., the mixture is stirred at 15° C. for 16 hrs. LCMS showed the desired MS is given. The mixture is concentrated under vacuum to give a residue, then the mixture is diluted with DCM (2000 mL), washed with 1 N HCl aqueous solution (700 mL*2), then saturated NaHCO₃ aqueous solution (700 mL*2) and concentrated under vacuum. The crude is purified by column chromatography to give Compound 3C (304 g, 155 mmol, 63.1% yield, 96.0% purity) as a yellow solid

### General Procedure for Preparation of Compound 4C

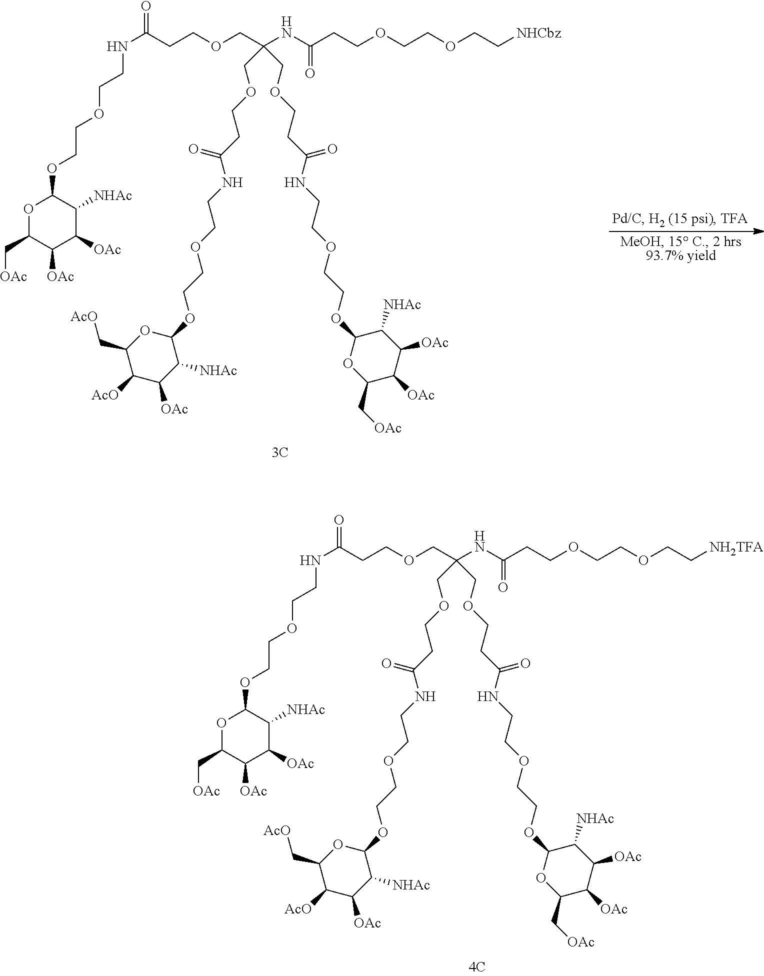

Two batches solution of Compound 3C (55.0 g, 29.2 mmol, 1.00 eq) in MeOH (1600 mL) is added Pd/C (6.60 g, 19.1 mmol, 10.0% purity) and TFA (3.34 g, 29.2 mmol, 2.17 mL, 1.00 eq), the mixture is degassed under vacuum and purged with H₂. The mixture is stirred under H₂ (15 psi) at 15° C. for 2 hours. LCMS showed the desired MS is given. The mixture is filtered and the filtrate is concentrated under vacuum to give Compound 4C (106 g, 54.8 mmol, 93.7% yield, 96.2% purity, TFA) as a white solid.

### General Procedure for Preparation of Compound 5C

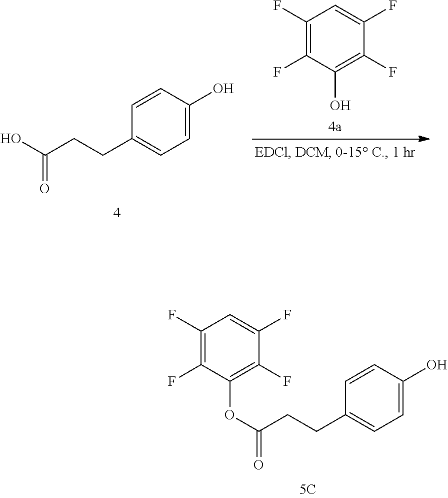 [SMILES: O=C(O)CCc1ccc(O)cc1 ; Oc1c(F)c(F)cc(F)c1F ; O=C(CCc1ccc(O)cc1)Oc1c(F)c(F)cc(F)c1F]

Two batches in parallel. To a solution of EDCI (28.8 g, 150 mmol, 1.00 eq) in DCM (125 mL) is added compound 4a (25.0 g, 150 mmol, 1.00 eq) dropwise at 0° C., then the mixture is added to compound 4 (25.0 g, 150 mmol, 1.00 eq) in DCM (125 mL) at 0° C., then the mixture is stirred at 25° C. for 1 hr. TLC (Petroleum ether:Ethyl acetate=3:1, R_{f}=0.45) showed the reactant is consumed and one new spot is formed. The reaction mixture is diluted with DCM (100 mL) then washed with aq·NaHCO₃ (250 mL*1) and brine (250 mL), dried over Na₂SO₄, filtered and concentrated under reduced pressure to give a residue. The residue is purified by column chromatography (SiO₂, Petroleum ether:Ethyl acetate=100:1 to 3:1), TLC (SiO₂, Petroleum ether:Ethyl acetate=3:1), R_{f}=0.45, then concentrated under reduced pressure to give a residue. Compound 5C (57.0 g, 176 mmol, 58.4% yield, 96.9% purity) is obtained as colorless oil and confirmed ¹HNMR: EW33072-2-P1A, 400 MHz, DMSO δ 9.21 (s, 1H), 7.07-7.09 (m, 2H), 6.67-6.70 (m, 2H), 3.02-3.04 (m, 2H), 2.86-2.90 (m, 2H)

### General Procedure for Preparation of Compound 6

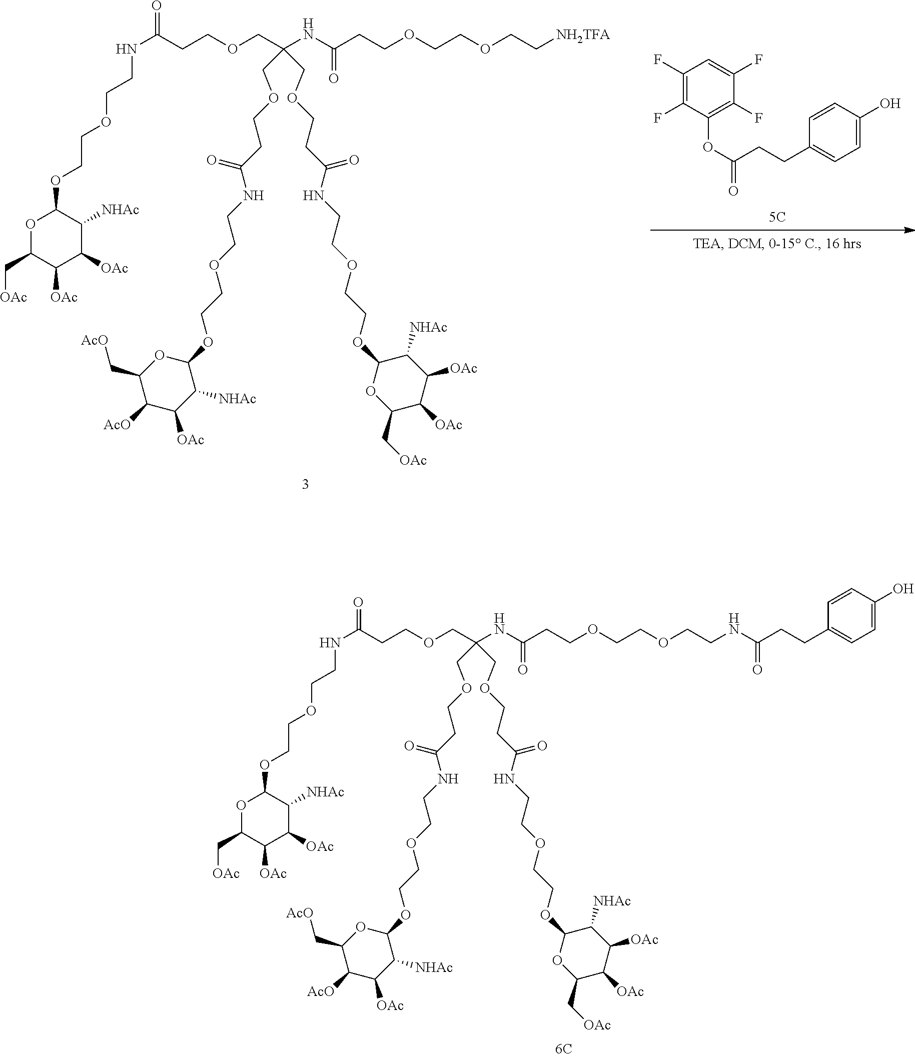

To a mixture of compound 3 (79.0 g, 41.0 mmol, 96.4% purity, 1.00 eq, TFA) and compound 6C (14.2 g, 43.8 mmol, 96.9% purity, 1.07 eq) in DCM (800 mL) is added TEA (16.6 g, 164 mmol, 22.8 mL, 4.00 eq) dropwise at 0° C., the mixture is stirred at 15° C. for 16 hrs. LCMS (EW33072-12-P1B, Rt=0.844 min) showed the desired mass is detected. The reaction mixture is diluted with DCM (400 mL) and washed with aq·NaHCO₃ (400 mL*1) and brine (400 mL*1), then the mixture is diluted with DCM (2.00 L) and washed with 0.7 M Na₂CO₃ (1000 mL*3) and brine (800 mL*3), dried over Na₂SO₄, filtered and concentrated under reduced pressure to give a residue. The residue is used to next step directly without purification. Compound 6 (80.0 g, crude) is obtained as white solid and confirmed via ¹HNMR: EW33072-12-P1A, 400 MHz, MeOD 6 7.02-7.04 (m, 2H), 6.68-6.70 (m, 2H), 5.34-5.35 (s, 3H), 5.07-5.08 (d, J=4.00 Hz, 3H), 4.62-4.64 (d, J=8.00 Hz, 3H), 3.71-4.16 (m, 16H), 3.31-3.70 (m, 44H), 2.80-2.83 (m, 2H), 2.68 (m, 2H), 2.46-2.47 (m, 10H), 2.14 (s, 9H), 2.03 (s, 9H), 1.94-1.95 (d, J=4.00 Hz, 18H).

### General Procedure for Preparation of TriGNal-TRIS-Peg2-Phosph 8c

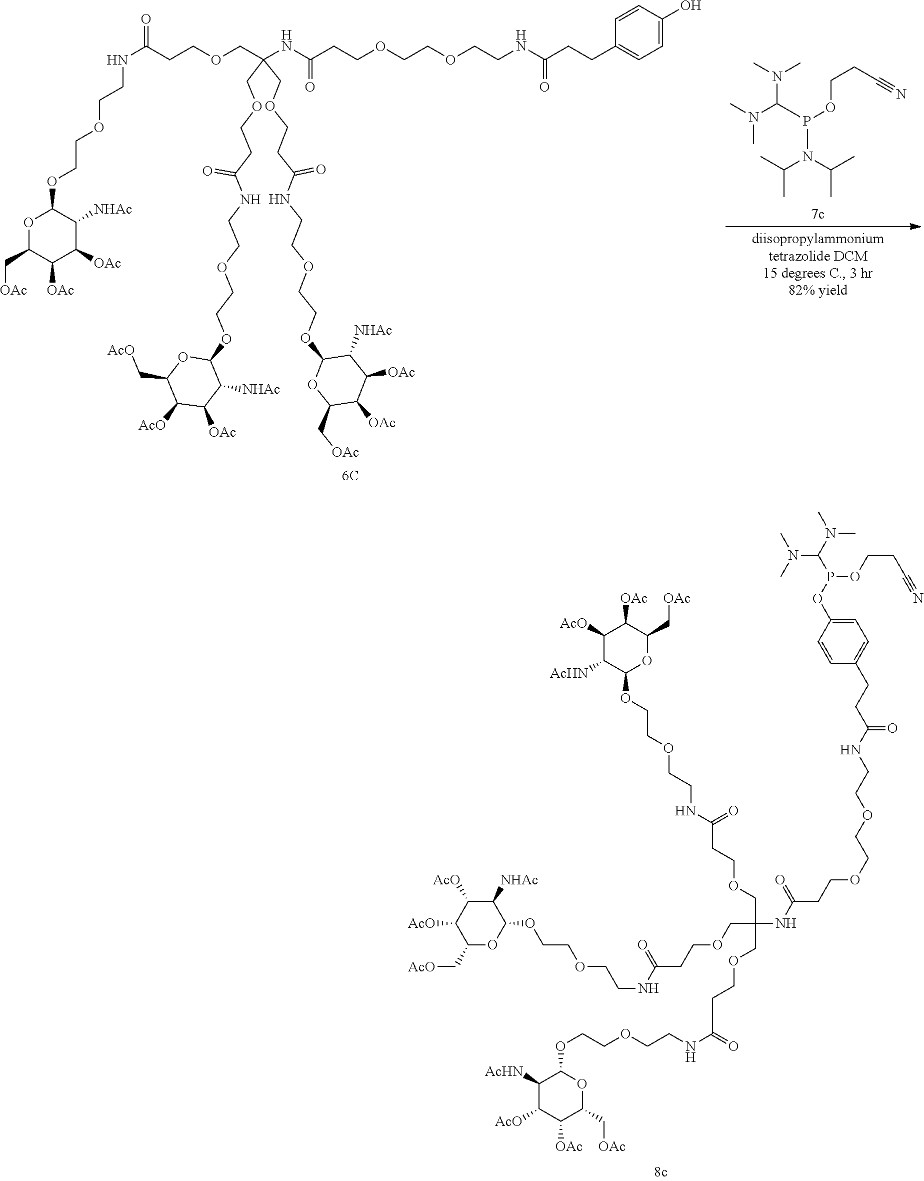

Two batches are synthesized in parallel. To a solution of compound 6C (40.0 g, 21.1 mmol, 1.00 eq in DCM (600 mL) is added diisopropylammonium tetrazolide (3.62 g, 21.1 mmol, 1.00 eq) and compound 7c (6.37 g, 21.1 mmol, 6.71 mL, 1.00 eq) in DCM (8.00 mL) drop-wise, the mixture is stirred at 30° C. for 1 hr, then added compound 7c (3.18 g, 10.6 mmol, 3.35 mL, 0.50 eq) in DCM (8.00 mL) drop-wise, the mixture is stirred at 30° C. for 30 mins, then added compound 7c (3.18 g, 10.6 mmol, 3.35 mL, 0.50 eq) in DCM (8.00 mL) drop-wise, the mixture is stirred at 30° C. for 1.5 hrs. LCMS (EW33072-17-P1C1, Rt=0.921 min) showed the desired MS+1 is detected. LCMS (EW33072-17-P1C2, Rt=0.919 min) showed the desired MS+1 is detected. Two batches are combined for work-up. The mixture is diluted with DCM (1.20 L), washed with saturated NaHCO₃ aqueous solution (1.60 L*2), 3% DMF in H₂O (1.60 L*2), H₂O (1.60 L*3), brine (1.60 L), dried over Na₂SO₄, filtered and concentrated under reduced pressure to give a residue. The residue is purified by column chromatography (SiO₂, DCM:MeOH:TEA=100:3:2) TLC (SiO₂, DCM:MeOH=10:1, R_{f}=0.45), then concentrated under reduced pressure to give a residue. Compound 8C (76.0 g, 34.8 mmol, 82.5% yield, 96.0% purity) is obtained as white solid and confirmed via ¹HNMR: EW33072-19-P1C, 400 MHz, MeOD δ 7.13-7.15 (d, J=8.50 Hz, 2H), 6.95-6.97 (dd, J=8.38, 1.13 Hz, 2H), 5.34 (d, J=2.88 Hz, 3H), 0.09 (dd, J=11.26, 3.38 Hz, 3H), 4.64 (d, J=8.50 Hz, 3H), 3.99-4.20 (m, 12H), 3.88-3.98 (m, 5H), 3.66-3.83 (m, 20H), 3.51-3.65 (m, 17H), 3.33-3.50 (m, 9H), 2.87 (t, J=7.63 Hz, 2H), 2.76 (t, J=5.94 Hz, 2H), 2.42-2.50 (m, 10H), 2.14 (s, 9H), 2.03 (s, 9H), 1.94-1.95 (d, J=6.13 Hz, 18H), 1.24-1.26 (d, J=6.75 Hz, 6H), 1.18-1.20 (d, J=6.75 Hz, 6H)

### Example 11: Modification Motif 1

An example GPAM siRNA includes a combination of the following modifications:
Position 9 (from 5′ to 3′) of the sense strand is 2′ F.
If position 9 is a pyrimidine then all purines in the Sense Strand are 2′OMe, and 1-5 pyrimidines between positions 5 and 11 are 2′ F provided that there are never three 2′F modifications in a row.
If position 9 is a purine then all pyrimidines in the Sense Strand are 2′OMe, and 1-5 purines between positions 5 and 11 are 2′ F provided that there are never three 2′F modifications in a row.
Antisense strand odd-numbered positions are 2′OMe and even-numbered positions are a mixture of 2′ F, 2′OMe and 2′ deoxy.

### Example 12: Modification Motif 2

An example GPAM siRNA includes a combination of the following modifications:
Position 9 (from 5′ to 3′) of the sense strand is 2′ deoxy.
Sense strand positions 5, 7 and 8 are 2′ F.
All pyrimidines in positions 10-21 are 2′ OMe, and purines are a mixture of 2′ OMe and 2′ F. Alternatively, all purines in positions 10-21 are 2′ OMe and all pyrimidines in positions 10-21 are a mixture of 2′ OMe and 2′ F.
Antisense strand odd-numbered positions are 2′OMe and even-numbered positions are a mixture of 2′ F, 2′OMe and 2′ deoxy.

### Example 12. Screening of siRNAs ETD01994-ETD02018 Targeting Human GPAM mRNA in Mice Transfected with AAV8-TBG-h-GPAM

The activities of siRNAs, namely ETD01994-ETD02018, were assessed in mice transiently expressing human GPAM. The siRNAs contain the GalNAc ligand ETL17 followed by a phosphorothioate linkage at the 5′ end of the sense strand. The siRNA sequences that were used are shown in Table 14, where Nf (e.g. Af, Cf, Gf, Tf, or Uf) is a 2′ fluoro-modified nucleoside, n (e.g. a, c, g, t, or u) is a 2′ O-methyl modified nucleoside, dN (e.g. dA, dC, dG, dT, or dU) is a 2′ deoxynucleoside, and “s” is a phosphorothioate linkage. The base sequences for each siRNA, with and without the 3′ UU extension, are shown in Table 15. ETD01994-ETD02004 were tested in Part 1 of the study and ETD02005-ETD02018 were tested in Part 2.

Six to eight week old female mice (C57Bl/6) were injected with 5 uL of a recombinant adeno-associated virus 8 (AAV8) vector (1.3×10E13 genome copies/mL) by the retroorbital route on Day −14. The recombinant AAV8 contains the open reading frame, the 5′ UTR, and a portion of the 3′UTR of the human GPAM sequence (ENST00000348367) under the control of the human thyroxine binding globulin promoter in an AAV2 backbone packaged in AAV8 capsid (AAV8-TBG-h-GPAM). On Day 0, infected mice were given a subcutaneous injection of a single 100 ug dose of a GalNAc-conjugated siRNA or PBS as vehicle control (n=4).

Mice were euthanized on Day 10 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver GPAM mRNA were assessed by RT-qPCR in triplicate on a QuantStudio™ 6 Pro Real-Time PCR System using TaqMan assays for human GPAM (ThermoFisher, assay #Hs01573684_m1), or mouse GPAM (ThermoFisher, assay #Mm01261106_m1), and the mouse housekeeping gene PPIA (ThermoFisher, assay #Mm02342430_g1), and PerfeCTa® qPCR FastMix®, Low ROX™ (VWR, Catalog #101419-222). Only ETD01997, ETD01998, ETD02003, ETD02010, ETD02018 have highly similar target sequences in human and mouse GPAM. Data were normalized to the mean GPAM mRNA level in animals receiving a subcutaneous injection of PBS. Results for Part 1 are shown in Table 16, and those for Part 2 in Table 17. Of the siRNAs in this screening set, mice injected with ETD02009, ETD02011, ETD02012 or ETD02015 had the highest level of human GPAM mRNA knockdown in the liver.

TABLE 14
Example siRNA Sequences

| | SEQ ID | Sense Strand Sequence (5′-3′) | SEQ ID | |
|---|---|---|---|---|
| ETD# | NO | with GalNAc moiety | NO | Antisense Strand Sequence (5′-3′) |
| ETD01994 | 12868 | [ETL17]saucaGfAfauAfcAfguguuggasusu | 12975 | usCfscAfaCfaCfuGfuAfuUfcUfgAfususu |
| ETD01995 | 12869 | [ETL17]sgaauaCfagUfgUfuggucgaasusu | 12976 | usUfscGfaCfcAfaCfaCfuGfuAfuUfcsusu |
| ETD01996 | 12870 | [ETL17]scuuugGfGfUfdTugcggaaugasusu | 12977 | usCfsaUfuCfcGfcAfaAfcCfcAfaAfgsusu |
| ETD01997 | 12871 | [ETL17]sauaaGfGfgcAfuGfuuugccaasusu | 12978 | usUfsgGfcAfaAfcAfuGfcCfcUfuAfususu |
| ETD01998 | 12872 | [ETL17]sagcugCfaaCfUfgagacgaaasusu | 12979 | usUfsuCfgUfcUfcAfgUfuGfcAfgCfususu |
| ETD01999 | 12873 | [ETL17]sucuaCfCfagUfuCfauagaucasusu | 12980 | usGfsaUfcUfaUfgAfaCfuGfgUfaGfasusu |
| ETD02000 | 12874 | [ETL17]scuacCfagUfUfcauagauccasusu | 12981 | usGfsgAfuCfuAfuGfaAfcUfgGfuAfgsusu |
| ETD02001 | 12875 | [ETL17]scuugAfuccAfuAfagcuuggasusu | 12982 | usCfscAfaGfcUfuAfuGfgAfuCfaAfgsusu |
| ETD02002 | 12876 | [ETL17]sacggAfAfaGfAfuGfuucucuaasusu | 12983 | usUfsaGfaGfaAfcAfuCfuUfuCfcGfususu |
| ETD02003 | 12877 | [ETL17]succaUfgggCfaUfauaguugasusu | 12984 | usCfsaAfcUfaUfaUfgCfcCfaUfgGfasusu |
| ETD02004 | 12878 | [ETL17]suauaGfuuGfAfauuacuucgasusu | 12985 | usCfsgAfaGfuAfaUfuCfaAfcUfaUfasusu |
| ETD02005 | 12879 | [ETL17]sauaguUfGfAfdAuuacuucgaasusu | 12986 | usUfscGfaAfgUfaAfuUfcAfaCfuAfususu |
| ETD02006 | 12880 | [ETL17]saguugaaUfUfaCfuucgacaasusu | 12987 | usUfsgUfcGfaAfgUfaAfuUfcAfaCfususu |
| ETD02007 | 12881 | [ETL17]suucgaCfAfGfdCagcaauucuasusu | 12988 | usAfsgAfaUfuGfcUfgCfuGfuCfgAfasusu |
| ETD02008 | 12882 | [ETL17]saguugUfGfGfdTagauacucuasusu | 12989 | usAfsgAfgUfaUfcUfaCfcAfcAfaCfususu |
| ETD02009 | 12883 | [ETL17]suggaaUfCfuCfCfuaugaucgasusu | 12990 | usCfsgAfuCfaUfaGfgAfgAfuUfcCfasusu |
| ETD02010 | 12884 | [ETL17]sucgaaggUfCfaCfuacaaugasusu | 12991 | usCfsaUfuGfuAfgUfgAfcCfuUfcGfasusu |
| ETD02011 | 12885 | [ETL17]suguuAfuuAfGfaAfuguuacgasusu | 12992 | usCfsgUfaAfcAfuUfcUfaAfuAfaCfasusu |
| ETD02012 | 12886 | [ETL17]saaacuAfuGfGfuuguguccgasusu | 12993 | usCfsgGfaCfaCfaAfcCfaUfaGfuUfususu |
| ETD02013 | 12887 | [ETL17]saagcgUfUfgUfUfaccagcuaasusu | 12994 | usUfsaGfcUfgGfuAfaCfaAfcGfcUfususu |
| ETD02014 | 12888 | [ETL17]suauucAfCfUfdGcuagcaaguasusu | 12995 | usAfscUfuGfcUfaGfcAfgUfgAfaUfasusu |
| ETD02015 | 12889 | [ETL17]sauucaCfUfgCfUfagcaagucasusu | 12996 | usGfsaCfuUfgCfuAfgCfaGfuGfaAfususu |
| ETD02016 | 12890 | [ETL17]suuauGfuccAfcAfcacauugasusu | 12997 | usCfsaAfuGfuGfuGfuGfgAfcAfuAfasusu |
| ETD02017 | 12891 | [ETL17]saacaGfAfaAfGfaAfauguugcasusu | 12998 | usGfscAfaCfaUfuUfcUfuUfcUfgUfususu |
| ETD02018 | 12892 | [ETL17]sauauaUfUfCfUfgaguuuuguasusu | 12999 | usAfscAfaAfaCfuCfaGfaAfuAfuAfususu |

TABLE 15
Example siRNA Base Sequences

| siRNA | SEQ | | SEQ ID | |
|---|---|---|---|---|
| Name | ID NO: | | NO: | |
| | | Sense Strand Base | | Antisense Strand Base |
| | | Sequence (5′ to 3′) | | Sequence (5′ to 3′) |
| ETD01994 | 13082 | AUCAGAAUACAGUGUUGGAUU | 13403 | UCCAACACUGUAUUCUGAUUU |
| ETD01995 | 13083 | GAAUACAGUGUUGGUCGAAUU | 13404 | UUCGACCAACACUGUAUUCUU |
| ETD01996 | 13084 | CUUUGGGUUUGCGGAAUGAUU | 13405 | UCAUUCCGCAAACCCAAAGUU |
| ETD01997 | 13085 | AUAAGGGCAUGUUUGCCAAUU | 13406 | UUGGCAAACAUGCCCUUAUUU |
| ETD01998 | 13086 | AGCUGCAACUGAGACGAAAUU | 13407 | UUUCGUCUCAGUUGCAGCUUU |
| ETD01999 | 13087 | UCUACCAGUUCAUAGAUCAUU | 13408 | UGAUCUAUGAACUGGUAGAUU |
| ETD02000 | 13088 | CUACCAGUUCAUAGAUCCAUU | 13409 | UGGAUCUAUGAACUGGUAGUU |
| ETD02001 | 13089 | CUUGAUCCAUAAGCUUGGAUU | 13410 | UCCAAGCUUAUGGAUCAAGUU |
| ETD02002 | 13090 | ACGGAAAGAUGUUCUCUAAUU | 13411 | UUAGAGAACAUCUUUCCGUUU |
| ETD02003 | 13091 | UCCAUGGGCAUAUAGUUGAUU | 13412 | UCAACUAUAUGCCCAUGGAUU |
| ETD02004 | 13092 | UAUAGUUGAAUUACUUCGAUU | 13413 | UCGAAGUAAUUCAACUAUAUU |
| ETD02005 | 13093 | AUAGUUGAAUUACUUCGAAUU | 13414 | UUCGAAGUAAUUCAACUAUUU |
| ETD02006 | 13094 | AGUUGAAUUACUUCGACAAUU | 13415 | UUGUCGAAGUAAUUCAACUUU |
| ETD02007 | 13095 | UUCGACAGCAGCAAUUCUAUU | 13416 | UAGAAUUGCUGCUGUCGAAUU |
| ETD02008 | 13096 | AGUUGUGGUAGAUACUCUAUU | 13417 | UAGAGUAUCUACCACAACUUU |
| ETD02009 | 13097 | UGGAAUCUCCUAUGAUCGAUU | 13418 | UCGAUCAUAGGAGAUUCCAUU |
| ETD02010 | 13098 | UCGAAGGUCACUACAAUGAUU | 13419 | UCAUUGUAGUGACCUUCGAUU |
| ETD02011 | 13099 | UGUUAUUAGAAUGUUACGAUU | 13420 | UCGUAACAUUCUAAUAACAUU |
| ETD02012 | 13100 | AAACUAUGGUUGUGUCCGAUU | 13421 | UCGGACACAACCAUAGUUUUU |
| ETD02013 | 13101 | AAGCGUUGUUACCAGCUAAUU | 13422 | UUAGCUGGUAACAACGCUUUU |
| ETD02014 | 13102 | UAUUCACUGCUAGCAAGUAUU | 13423 | UACUUGCUAGCAGUGAAUAUU |
| ETD02015 | 13103 | AUUCACUGCUAGCAAGUCAUU | 13424 | UGACUUGCUAGCAGUGAAUUU |
| ETD02016 | 13104 | UUAUGUCCACACACAUUGAUU | 13425 | UCAAUGUGUGUGGACAUAAUU |
| ETD02017 | 13105 | AACAGAAAGAAAUGUUGCAUU | 13426 | UGCAACAUUUCUUUCUGUUUU |
| ETD02018 | 13106 | AUAUAUUCUGAGUUUUGUAUU | 13427 | UACAAAACUCAGAAUAUAUUU |
| | | Sense Strand Base | | Antisense Strand Base |
| | | Sequence (5′ to 3′), | | Sequence (5′ to 3′), |
| | | without 3′ overhangs | | without 3′ overhangs |
| ETD01994 | 13107 | AUCAGAAUACAGUGUUGGA | 13428 | UCCAACACUGUAUUCUGAU |
| ETD01995 | 13108 | GAAUACAGUGUUGGUCGAA | 13429 | UUCGACCAACACUGUAUUC |
| ETD01996 | 13109 | CUUUGGGUUUGCGGAAUGA | 13430 | UCAUUCCGCAAACCCAAAG |
| ETD01997 | 13110 | AUAAGGGCAUGUUUGCCAA | 13431 | UUGGCAAACAUGCCCUUAU |
| ETD01998 | 13111 | AGCUGCAACUGAGACGAAA | 13432 | UUUCGUCUCAGUUGCAGCU |
| ETD01999 | 13112 | UCUACCAGUUCAUAGAUCA | 13433 | UGAUCUAUGAACUGGUAGA |
| ETD02000 | 13113 | CUACCAGUUCAUAGAUCCA | 13434 | UGGAUCUAUGAACUGGUAG |
| ETD02001 | 13114 | CUUGAUCCAUAAGCUUGGA | 13435 | UCCAAGCUUAUGGAUCAAG |
| ETD02002 | 13115 | ACGGAAAGAUGUUCUCUAA | 13436 | UUAGAGAACAUCUUUCCGU |
| ETD02003 | 13116 | UCCAUGGGCAUAUAGUUGA | 13437 | UCAACUAUAUGCCCAUGGA |
| ETD02004 | 13117 | UAUAGUUGAAUUACUUCGA | 13438 | UCGAAGUAAUUCAACUAUA |
| ETD02005 | 13118 | AUAGUUGAAUUACUUCGAA | 13439 | UUCGAAGUAAUUCAACUAU |
| ETD02006 | 13119 | AGUUGAAUUACUUCGACAA | 13440 | UUGUCGAAGUAAUUCAACU |
| ETD02007 | 13120 | UUCGACAGCAGCAAUUCUA | 13441 | UAGAAUUGCUGCUGUCGAA |
| ETD02008 | 13121 | AGUUGUGGUAGAUACUCUA | 13442 | UAGAGUAUCUACCACAACU |
| ETD02009 | 13122 | UGGAAUCUCCUAUGAUCGA | 13443 | UCGAUCAUAGGAGAUUCCA |
| ETD02010 | 13123 | UCGAAGGUCACUACAAUGA | 13444 | UCAUUGUAGUGACCUUCGA |
| ETD02011 | 13124 | UGUUAUUAGAAUGUUACGA | 13445 | UCGUAACAUUCUAAUAACA |
| ETD02012 | 13125 | AAACUAUGGUUGUGUCCGA | 13446 | UCGGACACAACCAUAGUUU |
| ETD02013 | 13126 | AAGCGUUGUUACCAGCUAA | 13447 | UUAGCUGGUAACAACGCUU |
| ETD02014 | 13127 | UAUUCACUGCUAGCAAGUA | 13448 | UACUUGCUAGCAGUGAAUA |
| ETD02015 | 13128 | AUUCACUGCUAGCAAGUCA | 13449 | UGACUUGCUAGCAGUGAAU |
| ETD02016 | 13129 | UUAUGUCCACACACAUUGA | 13450 | UCAAUGUGUGUGGACAUAA |
| ETD02017 | 13130 | AACAGAAAGAAAUGUUGCA | 13451 | UGCAACAUUUCUUUCUGUU |
| ETD02018 | 13131 | AUAUAUUCUGAGUUUUGUA | 13452 | UACAAAACUCAGAAUAUAU |
| ETD01994 | 13132 | AUCAGAAUACAGUGUUGG | 13453 | CCAACACUGUAUUCUGAU |
| ETD01995 | 13133 | GAAUACAGUGUUGGUCGA | 13454 | UCGACCAACACUGUAUUC |
| ETD01996 | 13134 | CUUUGGGUUUGCGGAAUG | 13455 | CAUUCCGCAAACCCAAAG |
| ETD01997 | 13135 | AUAAGGGCAUGUUUGCCA | 13456 | UGGCAAACAUGCCCUUAU |
| ETD01998 | 13136 | AGCUGCAACUGAGACGAA | 13457 | UUCGUCUCAGUUGCAGCU |
| ETD01999 | 13137 | UCUACCAGUUCAUAGAUC | 13458 | GAUCUAUGAACUGGUAGA |
| ETD02000 | 13138 | CUACCAGUUCAUAGAUCC | 13459 | GGAUCUAUGAACUGGUAG |
| ETD02001 | 13139 | CUUGAUCCAUAAGCUUGG | 13460 | CCAAGCUUAUGGAUCAAG |
| ETD02002 | 13140 | ACGGAAAGAUGUUCUCUA | 13461 | UAGAGAACAUCUUUCCGU |
| ETD02003 | 13141 | UCCAUGGGCAUAUAGUUG | 13462 | CAACUAUAUGCCCAUGGA |
| ETD02004 | 13142 | UAUAGUUGAAUUACUUCG | 13463 | CGAAGUAAUUCAACUAUA |
| ETD02005 | 13143 | AUAGUUGAAUUACUUCGA | 13464 | UCGAAGUAAUUCAACUAU |
| ETD02006 | 13144 | AGUUGAAUUACUUCGACA | 13465 | UGUCGAAGUAAUUCAACU |
| ETD02007 | 13145 | UUCGACAGCAGCAAUUCU | 13466 | AGAAUUGCUGCUGUCGAA |
| ETD02008 | 13146 | AGUUGUGGUAGAUACUCU | 13467 | AGAGUAUCUACCACAACU |
| ETD02009 | 13147 | UGGAAUCUCCUAUGAUCG | 13468 | CGAUCAUAGGAGAUUCCA |
| ETD02010 | 13148 | UCGAAGGUCACUACAAUG | 13469 | CAUUGUAGUGACCUUCGA |
| ETD02011 | 13149 | UGUUAUUAGAAUGUUACG | 13470 | CGUAACAUUCUAAUAACA |
| ETD02012 | 13150 | AAACUAUGGUUGUGUCCG | 13471 | CGGACACAACCAUAGUUU |
| ETD02013 | 13151 | AAGCGUUGUUACCAGCUA | 13472 | UAGCUGGUAACAACGCUU |
| ETD02014 | 13152 | UAUUCACUGCUAGCAAGU | 13473 | ACUUGCUAGCAGUGAAUA |
| ETD02015 | 13153 | AUUCACUGCUAGCAAGUC | 13474 | GACUUGCUAGCAGUGAAU |
| ETD02016 | 13154 | UUAUGUCCACACACAUUG | 13475 | CAAUGUGUGUGGACAUAA |
| ETD02017 | 13155 | AACAGAAAGAAAUGUUGC | 13476 | GCAACAUUUCUUUCUGUU |
| ETD02018 | 13156 | AUAUAUUCUGAGUUUUGU | 13477 | ACAAAACUCAGAAUAUAU |

TABLE 16
Relative GPAM mRNA Levels in Livers of Mice
Transfected with AAV8-TGB-h-GPAM - Part 1

| | | | | Mean GPAM mRNA | |
|---|---|---|---|---|---|
| | | | | (Normalized to Group 1, Day 10) | |
| | | | Dose | Human | Mouse |
| Group | n | Treatment | (ug) | GPAM mRNA | GPAM mRNA |
| 1 | 4 | PBS | | 1.00 | 1.00 |
| 2 | 4 | ETD01994 | 100 | 1.02 | 0.98 |
| 3 | 4 | ETD01995 | 100 | 1.31 | 1.96 |
| 4 | 4 | ETD01996 | 100 | 1.13 | 1.91 |
| 5 | 4 | ETD01997 | 100 | 0.85 | 0.76 |
| 6 | 4 | ETD01998 | 100 | 1.05 | 1.29 |
| 7 | 4 | ETD01999 | 100 | 0.85 | 0.96 |
| 8 | 4 | ETD02000 | 100 | 0.37 | 1.15 |
| 9 | 4 | ETD02001 | 100 | 0.68 | 0.99 |
| 10 | 4 | ETD02002 | 100 | 0.64 | 0.98 |
| 11 | 4 | ETD02003 | 100 | 0.96 | 1.03 |
| 12 | 4 | ETD02004 | 100 | 1.43 | 0.88 |

TABLE 17
Relative GPAM mRNA Levels in Livers of Mice
Transfected with AAV8-TGB-h-GPAM - Part 2

| | | | | Mean GPAM mRNA | |
|---|---|---|---|---|---|
| | | | | (Normalized to Group 1, Day 10) | |
| | | | Dose | Human | Mouse |
| Group | n | Treatment | (ug) | GPAM mRNA | GPAM mRNA |
| 1 | 4 | PBS | | 1.00 | 1.00 |
| 2 | 4 | ETD02005 | 100 | 0.33 | 1.27 |
| 3 | 4 | ETD02006 | 100 | 0.70 | 1.05 |
| 4 | 4 | ETD02007 | 100 | 0.44 | 0.75 |
| 5 | 4 | ETD02008 | 100 | 0.29 | 0.90 |
| 6 | 4 | ETD02009 | 100 | 0.07 | 0.93 |
| 7 | 4 | ETD02010 | 100 | 0.60 | 1.20 |
| 8 | 4 | ETD02011 | 100 | 0.08 | 0.94 |
| 9 | 4 | ETD02012 | 100 | 0.01 | 0.86 |
| 10 | 4 | ETD02013 | 100 | 0.34 | 0.83 |
| 11 | 4 | ETD02014 | 100 | 0.56 | 0.75 |
| 12 | 4 | ETD02015 | 100 | 0.18 | 0.76 |
| 13 | 4 | ETD02016 | 100 | 0.45 | 0.58 |
| 14 | 4 | ETD02017 | 100 | 0.49 | 0.72 |
| 15 | 4 | ETD02018 | 100 | 0.35 | 0.50 |

### Example 13. Screening siRNAs with Alternative Modification Patterns of ETD02012 and ETD02011 in Mice Transfected with AAV8-TBG-h-GPAM

The base sequences of ETD02012 and ETD02011 were synthesized to generate siRNAs (ETD02012, ETD02285-ETD02292 and ETD02011, ETD02301-ETD02309, respectively) with alternative modification patterns. The activities of the siRNAs were assessed using mice transiently expressing human GRAM. The siRNAs contain the GalNAc ligand ETL17 followed by a phosphorothioate linkage at the 5′ end of the sense strand. The siRNA sequences that were used are shown in Table 18, where Nf (e.g. Af, Cf, Gf, Tf, or Uf) is a 2′ fluoro-modified nucleoside, n (e.g. a, c, g, t, or u) is a 2′ O-methyl modified nucleoside, Nm (e.g. Am, Cm, Gm, Tm, or Um) is a 2′-O-(2-methoxyethyl) modified nucleoside, dN (e.g. dA, dC, dG, dT, or dU) is a 2′ deoxynucleoside, and “s” is a phosphorothioate linkage. The base sequences for each siRNA, with and without the 3′ UU extension, are shown in Table 19.

Six to eight week old female mice (C57Bl/6) were injected with 5 uL of a recombinant adeno-associated virus 8 (AAV8) vector (1.2×10E13 genome copies/mL) by the retroorbital route on Day −14. The recombinant AAV8 contains the open reading frame, the 5′ UTR, and a portion of the 3′UTR of the human GPAM sequence (ENST00000348367) under the control of the human thyroxine binding globulin promoter in an AAV2 backbone packaged in AAV8 capsid (AAV8-TBG-h-GPAM). On Day 0, infected mice were given a subcutaneous injection of a single 100 ug dose of a GalNAc-conjugated siRNA or PBS as vehicle control (n=5).

Mice were euthanized on Day 14 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver GPAM mRNA were assessed by RT-qPCR in triplicate on a QuantStudio™ 6 Pro Real-Time PCR System using TaqMan assays for human GPAM (ThermoFisher, assay #Hs01573684_m1) and the mouse housekeeping gene PPIA (ThermoFisher, assay #Mm02342430_g1), and PerfeCTa® qPCR FastMix®, Low ROX™ (VWR, Catalog #101419-222). Mice that gave low liver expression of human GPAM, as defined by having a Ct value of >30, were omitted from further analysis. Data were normalized to the mean GPAM mRNA level in animals receiving a subcutaneous injection of PBS. Results are shown in Table 20. Of the alternatively modified versions of ETD02012, ETD02289 had the greatest activity. Of the alternatively modified versions of ETD02011, ETD02304 and ETD02309 had the greatest activity.

TABLE 18
Example siRNA Sequences

| | SEQ | | SEQ | |
|---|---|---|---|---|
| | ID | Sense Strand Sequence (5′-3′) | ID | |
| ETD# | NO: | with GalNAc moiety | NO: | Antisense Strand Sequence (5′-3′) |
| ETD02012 | 12886 | [ETL17]saaacuAfuGfGfuuguguccgasusu | 12993 | usCfsgGfaCfaCfaAfcCfaUfaGfuUfususu |
| ETD02285 | 12893 | [ETL17]saaacuAfUfGfGfuugugUmccgasusu | 13000 | usCfsgGfaCfaCfaAfcCfaUfaGfuUfususu |
| ETD02286 | 12894 | [ETL17]saaacuAfuGfGfuuguguccgasusu | 13001 | usCfsggaCfaCfaAfcCfaUfaGfuUfususu |
| ETD02287 | 12895 | [ETL17]saaacuAfuGfGfuuguguccgasusu | 13002 | usCfsgGfaCfaCfaAfccaUfaGfuUfususu |
| ETD02288 | 12896 | [ETL17]saaacuAfuGfGfuuguguccgasusu | 13003 | usCfsgGfaCfacAfaccaUfaGfuUfususu |
| ETD02289 | 12897 | [ETL17]saaacuAfuGfGfuuguguccgasusu | 13004 | usCfsgGfaCfacAfaCfcaUfaGfuUfususu |
| ETD02290 | 12898 | [ETL17]saaacuAfuGfGfuuguguccgasusu | 13005 | usCfsgGfacAfcAfaCfcaUfaGfuUfususu |
| ETD02291 | 12899 | [ETL17]saaacuAfuGfGfuuguguccgasusu | 13006 | usCfsggAfcAfcAfaCfcaUfaGfuUfususu |
| ETD02292 | 12900 | [ETL17]saaacUmAfUfGfGfuugUmguccgasusu | 13007 | usCfsgGfaCfaCfaAfcCfaUfaGfuUfususu |
| ETD02011 | 12885 | [ETL17]suguuAfuuAfGfaAfuguuacgasusu | 12992 | usCfsgUfaAfcAfuUfcUfaAfuAfaCfasusu |
| ETD02301 | 12901 | [ETL17]suguuauuAfGfAfAfuguuacgasusu | 13008 | usCfsgUfaAfcAfuUfcUfaAfuAfaCfasusu |
| ETD02302 | 12902 | [ETL17]suguuAfuuAfGfaAfuguuacgasusu | 13009 | usCfsguaAfcAfuUfcUfaAfuAfaCfasusu |
| ETD02303 | 12903 | [ETL17]suguuAfuuAfGfaAfuguuacgasusu | 13010 | usCfsgUfaAfcAfuUfcuaAfuAfaCfasusu |
| ETD02304 | 12904 | [ETL17]suguuAfuuAfGfaAfuguuacgasusu | 13011 | usCfsgUfaAfcAfuucUfaAfuAfaCfasusu |
| ETD02305 | 12905 | [ETL17]suguuAfuuAfGfaAfuguuacgasusu | 13012 | usCfsguaAfcAfuucUfaAfuAfaCfasusu |
| ETD02306 | 12906 | [ETL17]suguuAfuuAfGfaAfuguuacgasusu | 13013 | usCfsguaAfcAfuUfcuaAfuAfaCfasusu |
| ETD02307 | 12907 | [ETL17]suguuAfuuAfGfaAfuguuacgasusu | 13014 | usCfsgUfaaCfauUfcUfaAfuAfaCfasusu |
| ETD02308 | 12908 | [ETL17]suguuAfuuAfGfaAfuguuacgasusu | 13015 | usCfsgUfaaCfauUfcuaAfuAfaCfasusu |
| ETD02309 | 12909 | [ETL17]suguuAfuuAfGfaAfuguuacgasusu | 13016 | usCfsguAfaCfauUfcUfaAfuAfaCfasusu |

TABLE 19
Example siRNA BASE Sequences

| siRNA | SEQ ID | Sense Strand Base | SEQ ID | Antisense Strand Base |
|---|---|---|---|---|
| Name | NO: | Sequence (5′ to 3′) | NO: | Sequence (5′ to 3′) |
| ETD02012 | 13100 | AAACUAUGGUUGUGUCCGAUU | 13421 | UCGGACACAACCAUAGUUUUU |
| ETD02285 | 13157 | AAACUAUGGUUGUGTCCGAUU | 13478 | UCGGACACAACCAUAGUUUUU |
| ETD02286 | 13158 | AAACUAUGGUUGUGUCCGAUU | 13479 | UCGGACACAACCAUAGUUUUU |
| ETD02287 | 13159 | AAACUAUGGUUGUGUCCGAUU | 13480 | UCGGACACAACCAUAGUUUUU |
| ETD02288 | 13160 | AAACUAUGGUUGUGUCCGAUU | 13481 | UCGGACACAACCAUAGUUUUU |
| ETD02289 | 13161 | AAACUAUGGUUGUGUCCGAUU | 13482 | UCGGACACAACCAUAGUUUUU |
| ETD02290 | 13162 | AAACUAUGGUUGUGUCCGAUU | 13483 | UCGGACACAACCAUAGUUUUU |
| ETD02291 | 13163 | AAACUAUGGUUGUGUCCGAUU | 13484 | UCGGACACAACCAUAGUUUUU |
| ETD02292 | 13164 | AAACUAUGGUUGUGUCCGAUU | 13485 | UCGGACACAACCAUAGUUUUU |
| ETD02011 | 13099 | UGUUAUUAGAAUGUUACGAUU | 13420 | UCGUAACAUUCUAAUAACAUU |
| ETD02301 | 13165 | UGUUAUUAGAAUGUUACGAUU | 13486 | UCGUAACAUUCUAAUAACAUU |
| ETD02302 | 13166 | UGUUAUUAGAAUGUUACGAUU | 13487 | UCGUAACAUUCUAAUAACAUU |
| ETD02303 | 13167 | UGUUAUUAGAAUGUUACGAUU | 13488 | UCGUAACAUUCUAAUAACAUU |
| ETD02304 | 13168 | UGUUAUUAGAAUGUUACGAUU | 13489 | UCGUAACAUUCUAAUAACAUU |
| ETD02305 | 13169 | UGUUAUUAGAAUGUUACGAUU | 13490 | UCGUAACAUUCUAAUAACAUU |
| ETD02306 | 13170 | UGUUAUUAGAAUGUUACGAUU | 13491 | UCGUAACAUUCUAAUAACAUU |
| ETD02307 | 13171 | UGUUAUUAGAAUGUUACGAUU | 13492 | UCGUAACAUUCUAAUAACAUU |
| ETD02308 | 13172 | UGUUAUUAGAAUGUUACGAUU | 13493 | UCGUAACAUUCUAAUAACAUU |
| ETD02309 | 13173 | UGUUAUUAGAAUGUUACGAUU | 13494 | UCGUAACAUUCUAAUAACAUU |
| | | Sense Strand Base | | Antisense Strand Base |
| siRNA | | Sequence (5′ to 3′), | | Sequence (5′ to 3′), |
| Name | | without 3′ overhangs | | without 3′ overhangs |
| ETD02012 | 13125 | AAACUAUGGUUGUGUCCGA | 13446 | UCGGACACAACCAUAGUUU |
| ETD02285 | 13174 | AAACUAUGGUUGUGUCCGA | 13495 | UCGGACACAACCAUAGUUU |
| ETD02286 | 13175 | AAACUAUGGUUGUGUCCGA | 13496 | UCGGACACAACCAUAGUUU |
| ETD02287 | 13176 | AAACUAUGGUUGUGUCCGA | 13497 | UCGGACACAACCAUAGUUU |
| ETD02288 | 13177 | AAACUAUGGUUGUGUCCGA | 13498 | UCGGACACAACCAUAGUUU |
| ETD02289 | 13178 | AAACUAUGGUUGUGUCCGA | 13499 | UCGGACACAACCAUAGUUU |
| ETD02290 | 13179 | AAACUAUGGUUGUGUCCGA | 13500 | UCGGACACAACCAUAGUUU |
| ETD02291 | 13180 | AAACUAUGGUUGUGUCCGA | 13501 | UCGGACACAACCAUAGUUU |
| ETD02292 | 13181 | AAACUAUGGUUGUGUCCGA | 13502 | UCGGACACAACCAUAGUUU |
| ETD02011 | 13124 | UGUUAUUAGAAUGUUACGA | 13445 | UCGUAACAUUCUAAUAACA |
| ETD02301 | 13182 | UGUUAUUAGAAUGUUACGA | 13503 | UCGUAACAUUCUAAUAACA |
| ETD02302 | 13183 | UGUUAUUAGAAUGUUACGA | 13504 | UCGUAACAUUCUAAUAACA |
| ETD02303 | 13184 | UGUUAUUAGAAUGUUACGA | 13505 | UCGUAACAUUCUAAUAACA |
| ETD02304 | 13185 | UGUUAUUAGAAUGUUACGA | 13506 | UCGUAACAUUCUAAUAACA |
| ETD02305 | 13186 | UGUUAUUAGAAUGUUACGA | 13507 | UCGUAACAUUCUAAUAACA |
| ETD02306 | 13187 | UGUUAUUAGAAUGUUACGA | 13508 | UCGUAACAUUCUAAUAACA |
| ETD02307 | 13188 | UGUUAUUAGAAUGUUACGA | 13509 | UCGUAACAUUCUAAUAACA |
| ETD02308 | 13189 | UGUUAUUAGAAUGUUACGA | 13510 | UCGUAACAUUCUAAUAACA |
| ETD02309 | 13190 | UGUUAUUAGAAUGUUACGA | 13511 | UCGUAACAUUCUAAUAACA |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02012 | 13150 | AAACUAUGGUUGUGUCCG | 13471 | CGGACACAACCAUAGUUU |
| ETD02285 | 13191 | AAACUAUGGUUGUGUCCG | 13512 | CGGACACAACCAUAGUUU |
| ETD02286 | 13192 | AAACUAUGGUUGUGUCCG | 13513 | CGGACACAACCAUAGUUU |
| ETD02287 | 13193 | AAACUAUGGUUGUGUCCG | 13514 | CGGACACAACCAUAGUUU |
| ETD02288 | 13194 | AAACUAUGGUUGUGUCCG | 13515 | CGGACACAACCAUAGUUU |
| ETD02289 | 13195 | AAACUAUGGUUGUGUCCG | 13516 | CGGACACAACCAUAGUUU |
| ETD02290 | 13196 | AAACUAUGGUUGUGUCCG | 13517 | CGGACACAACCAUAGUUU |
| ETD02291 | 13197 | AAACUAUGGUUGUGUCCG | 13518 | CGGACACAACCAUAGUUU |
| ETD02292 | 13198 | AAACUAUGGUUGUGUCCG | 13519 | CGGACACAACCAUAGUUU |
| ETD02011 | 13149 | UGUUAUUAGAAUGUUACG | 13470 | CGUAACAUUCUAAUAACA |
| ETD02301 | 13199 | UGUUAUUAGAAUGUUACG | 13520 | CGUAACAUUCUAAUAACA |
| ETD02302 | 13200 | UGUUAUUAGAAUGUUACG | 13521 | CGUAACAUUCUAAUAACA |
| ETD02303 | 13201 | UGUUAUUAGAAUGUUACG | 13522 | CGUAACAUUCUAAUAACA |
| ETD02304 | 13202 | UGUUAUUAGAAUGUUACG | 13523 | CGUAACAUUCUAAUAACA |
| ETD02305 | 13203 | UGUUAUUAGAAUGUUACG | 13524 | CGUAACAUUCUAAUAACA |
| ETD02306 | 13204 | UGUUAUUAGAAUGUUACG | 13525 | CGUAACAUUCUAAUAACA |
| ETD02307 | 13205 | UGUUAUUAGAAUGUUACG | 13526 | CGUAACAUUCUAAUAACA |
| ETD02308 | 13206 | UGUUAUUAGAAUGUUACG | 13527 | CGUAACAUUCUAAUAACA |
| ETD02309 | 13207 | UGUUAUUAGAAUGUUACG | 13528 | CGUAACAUUCUAAUAACA |

TABLE 20
Relative GPAM mRNA Levels in Livers of
Mice Transfected with AAV8-TGB-h-GPAM

| | | | Dose | Mean human GPAM mRNA level |
|---|---|---|---|---|
| Group | n | Treatment | (ug) | (Normalized to Group 1, Day 14) |
| 1 | 5 | PBS | | 1.00 |
| 2 | 3 | ETD02012 | 100 | 0.48 |
| 3 | 2 | ETD02285 | 100 | 0.70 |
| 4 | 4 | ETD02286 | 100 | 0.82 |
| 5 | 3 | ETD02287 | 100 | 0.60 |
| 6 | 0 | ETD02288 | 100 | — |
| 7 | 4 | ETD02289 | 100 | 0.10 |
| 8 | 5 | ETD02290 | 100 | 0.82 |
| 9 | 3 | ETD02291 | 100 | 1.07 |
| 10 | 5 | ETD02292 | 100 | 0.77 |
| 11 | 5 | ETD02011 | 100 | 0.16 |
| 12 | 5 | ETD02301 | 100 | 0.22 |
| 13 | 4 | ETD02302 | 100 | 0.45 |
| 14 | 3 | ETD02303 | 100 | 0.27 |
| 15 | 4 | ETD02304 | 100 | 0.19 |
| 16 | 2 | ETD02305 | 100 | 0.35 |
| 17 | 3 | ETD02306 | 100 | 0.36 |
| 18 | 5 | ETD02307 | 100 | 0.24 |
| 19 | 5 | ETD02308 | 100 | 0.68 |
| 20 | 4 | ETD02309 | 100 | 0.19 |

### Example 14. Screening siRNAs with Alternative Modification Patterns of ETD02009 and ETD02015 in Mice Transfected with AAV8-TBG-h-GPAM

The base sequences of ETD02009 and ETD02015 were synthesized to generate siRNAs (ETD02293-ETD02300, and ETD02015-ETD02318, respectively) with alternative modification patterns. The activities of the siRNAs were assessed using mice transiently expressing human GPAM. The siRNAs contain the GalNAc ligand ETL17 followed by a phosphorothioate linkage at the 5′ end of the sense strand. The siRNA sequences that were used are shown in Table 21, where Nf (e.g. Af, Cf, Gf, Tf, or Uf) is a 2′ fluoro-modified nucleoside, n (e.g. a, c, g, t, or u) is a 2′ O-methyl modified nucleoside, Nm (e.g. Am, Cm, Gm, Tm, or Urn) is a 2′-O-(2-methoxyethyl) modified nucleoside, dN (e.g. dA, dC, dG, dT, or dU) is a 2′ deoxynucleoside, and “s” is a phosphorothioate linkage. The base sequences for each siRNA, with and without the 3′ UU extension, are shown in Table 22.

Six to eight week old female mice (C57Bl/6) were injected with 5 uL of a recombinant adeno-associated virus 8 (AAV8) vector (1.2×10E13 genome copies/mL) by the retroorbital route on Day −14. The recombinant AAV8 contains the open reading frame, the 5′ UTR, and a portion of the 3′UTR of the human GPAM sequence (ENST00000348367) under the control of the human thyroxine binding globulin promoter in an AAV2 backbone packaged in AAV8 capsid (AAV8-TBG-h-GPAM). On Day 0, infected mice were given a subcutaneous injection of a single 100 ug dose of a GalNAc-conjugated siRNA or PBS as vehicle control (n=5).

Mice were euthanized on Day 14 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver GPAM mRNA were assessed by RT-qPCR in triplicate on a QuantStudio™ 6 Pro Real-Time PCR System using TaqMan assays for human GPAM (ThermoFisher, assay #Hs01573684_m1) and the mouse housekeeping gene PPIA (ThermoFisher, assay #Mm02342430_g1), and PerfeCTa® qPCR FastMix®, Low ROX™ (VWR, Catalog #101419-222). Mice that gave low liver expression of human GPAM, as defined by having a Ct value of >30, were omitted from further analysis. Data were normalized to the mean GPAM mRNA level in animals receiving a subcutaneous injection of PBS. Results are shown in Table 23. Of the alternatively modified versions of ETD02009, ETD02296 had the greatest activity. Of the alternatively modified versions of ETD02015, ETD02318 had the greatest activity.

TABLE 21
Example siRNA Sequences

| | SEQ | | SEQ | |
|---|---|---|---|---|
| | ID | Sense Strand Sequence (5′-3′) with | ID | |
| ETD# | NO: | GalNAc moiety | NO: | Antisense Strand Sequence (5′-3′) |
| ETD02009 | 12883 | [ETL17]suggaaUfCfuCfCfuaugaucgasusu | 12990 | usCfsgAfuCfaUfaGfgAfgAfuUfcCfasusu |
| ETD02293 | 12910 | [ETL17]suggaaUfCfUfCfcuaugaucgasusu | 13017 | usCfsgAfuCfaUfaGfgAfgAfuUfcCfasusu |
| ETD02294 | 12911 | [ETL17]suggaaUfCfuCfCfuaugaucgasusu | 13018 | usCfsgauCfaUfaGfgAfgAfuUfcCfasusu |
| ETD02295 | 12912 | [ETL17]suggaaUfCfuCfCfuaugaucgasusu | 13019 | usCfsgAfucaUfaGfgAfgAfuUfcCfasusu |
| ETD02296 | 12913 | [ETL17]suggaaUfCfuCfCfuaugaucgasusu | 13020 | usCfsgAfuCfaUfaGfgagAfuUfcCfasusu |
| ETD02297 | 12914 | [ETL17]suggaaUfCfuCfCfuaugaucgasusu | 13021 | usCfsgauCfaUfaGfgagAfuUfcCfasusu |
| ETD02298 | 12915 | [ETL17]suggaaUfCfuCfCfuaugaucgasusu | 13022 | usCfsgAfucAfuaGfgAfgAfuUfcCfasusu |
| ETD02299 | 12916 | [ETL17]suggaaUfCfuCfCfuaugaucgasusu | 13023 | usCfsgaucAfuAfggAfgAfuUfcCfasusu |
| ETD02300 | 12917 | [ETL17]suggaaUfCfuCfCfuaugaucgasusu | 13024 | usCfsgAfucAfuAfggagAfuUfcCfasusu |
| ETD02015 | 12889 | [ETL17]sauucaCfUfgCfUfagcaagucasusu | 12996 | usGfsaCfuUfgCfuAfgCfaGfuGfaAfususu |
| ETD02310 | 12918 | [ETL17]sauucacUfgCfUfagcaagucasusu | 13025 | usGfsaCfuUfgCfuAfgCfaGfuGfaAfususu |
| ETD02311 | 12919 | [ETL17]sauucaCfUfgCfUfagcaagucasusu | 13026 | usGfsacuUfgCfuAfgCfaGfuGfaAfususu |
| ETD02312 | 12920 | [ETL17]sauucaCfUfgCfUfagcaagucasusu | 13027 | usGfsaCfuUfgCfuAfgcaGfuGfaAfususu |
| ETD02313 | 12921 | [ETL17]sauucaCfUfgCfUfagcaagucasusu | 13028 | usGfsacuUfgCfuAfgcaGfuGfaAfususu |
| ETD02314 | 12922 | [ETL17]sauucaCfUfgCfUfagcaagucasusu | 13029 | usGfsacuUfgCfuagCfaGfuGfaAfususu |
| ETD02315 | 12923 | [ETL17]sauucaCfUfgCfUfagcaagucasusu | 13030 | usGfsaCfuuGfcuAfgCfaGfuGfaAfususu |
| ETD02316 | 12924 | [ETL17]sauucaCfUfgCfUfagcaagucasusu | 13031 | usGfsacUfuGfcuAfgCfaGfuGfaAfususu |
| ETD02317 | 12925 | [ETL17]sauucaCfUfgCfUfagcaagucasusu | 13032 | usGfsacUfuGfcUfagCfaGfuGfaAfususu |
| ETD02318 | 12926 | [ETL17]sauucaCfUfgCfUfagcaagucasusu | 13033 | usGfsacUfuGfcUfagcaGfuGfaAfususu |

TABLE 22
Example siRNA Base Sequences

| siRNA | SEQ ID | Sense Strand Base Sequence | SEQ ID | Antisense Strand Base |
|---|---|---|---|---|
| Name | NO: | (5′ to 3′) | NO | Sequence (5′ to 3′) |
| ETD02009 | 13097 | UGGAAUCUCCUAUGAUCGAUU | 13418 | UCGAUCAUAGGAGAUUCCAUU |
| ETD02293 | 13208 | UGGAAUCUCCUAUGAUCGAUU | 13529 | UCGAUCAUAGGAGAUUCCAUU |
| ETD02294 | 13209 | UGGAAUCUCCUAUGAUCGAUU | 13530 | UCGAUCAUAGGAGAUUCCAUU |
| ETD02295 | 13210 | UGGAAUCUCCUAUGAUCGAUU | 13531 | UCGAUCAUAGGAGAUUCCAUU |
| ETD02296 | 13211 | UGGAAUCUCCUAUGAUCGAUU | 13532 | UCGAUCAUAGGAGAUUCCAUU |
| ETD02297 | 13212 | UGGAAUCUCCUAUGAUCGAUU | 13533 | UCGAUCAUAGGAGAUUCCAUU |
| ETD02298 | 13213 | UGGAAUCUCCUAUGAUCGAUU | 13534 | UCGAUCAUAGGAGAUUCCAUU |
| ETD02299 | 13214 | UGGAAUCUCCUAUGAUCGAUU | 13535 | UCGAUCAUAGGAGAUUCCAUU |
| ETD02300 | 13215 | UGGAAUCUCCUAUGAUCGAUU | 13536 | UCGAUCAUAGGAGAUUCCAUU |
| ETD02015 | 13103 | AUUCACUGCUAGCAAGUCAUU | 13424 | UGACUUGCUAGCAGUGAAUUU |
| ETD02310 | 13216 | AUUCACUGCUAGCAAGUCAUU | 13537 | UGACUUGCUAGCAGUGAAUUU |
| ETD02311 | 13217 | AUUCACUGCUAGCAAGUCAUU | 13538 | UGACUUGCUAGCAGUGAAUUU |
| ETD02312 | 13218 | AUUCACUGCUAGCAAGUCAUU | 13539 | UGACUUGCUAGCAGUGAAUUU |
| ETD02313 | 13219 | AUUCACUGCUAGCAAGUCAUU | 13540 | UGACUUGCUAGCAGUGAAUUU |
| ETD02314 | 13220 | AUUCACUGCUAGCAAGUCAUU | 13541 | UGACUUGCUAGCAGUGAAUUU |
| ETD02315 | 13221 | AUUCACUGCUAGCAAGUCAUU | 13542 | UGACUUGCUAGCAGUGAAUUU |
| ETD02316 | 13222 | AUUCACUGCUAGCAAGUCAUU | 13543 | UGACUUGCUAGCAGUGAAUUU |
| ETD02317 | 13223 | AUUCACUGCUAGCAAGUCAUU | 13544 | UGACUUGCUAGCAGUGAAUUU |
| ETD02318 | 13224 | AUUCACUGCUAGCAAGUCAUU | 13545 | UGACUUGCUAGCAGUGAAUUU |
| | | Sense Strand Base | | Antisense Strand Base |
| siRNA | SEQ ID | Sequence (5′ to 3′), | SEQ ID | Sequence (5′ to 3′), |
| Name | NO: | without 3′ overhangs | NO: | without 3′ overhangs |
| ETD02009 | 13122 | UGGAAUCUCCUAUGAUCGA | 13443 | UCGAUCAUAGGAGAUUCCA |
| ETD02293 | 13225 | UGGAAUCUCCUAUGAUCGA | 13546 | UCGAUCAUAGGAGAUUCCA |
| ETD02294 | 13226 | UGGAAUCUCCUAUGAUCGA | 13547 | UCGAUCAUAGGAGAUUCCA |
| ETD02295 | 13227 | UGGAAUCUCCUAUGAUCGA | 13548 | UCGAUCAUAGGAGAUUCCA |
| ETD02296 | 13228 | UGGAAUCUCCUAUGAUCGA | 13549 | UCGAUCAUAGGAGAUUCCA |
| ETD02297 | 13229 | UGGAAUCUCCUAUGAUCGA | 13550 | UCGAUCAUAGGAGAUUCCA |
| ETD02298 | 13230 | UGGAAUCUCCUAUGAUCGA | 13551 | UCGAUCAUAGGAGAUUCCA |
| ETD02299 | 13231 | UGGAAUCUCCUAUGAUCGA | 13552 | UCGAUCAUAGGAGAUUCCA |
| ETD02300 | 13232 | UGGAAUCUCCUAUGAUCGA | 13553 | UCGAUCAUAGGAGAUUCCA |
| ETD02015 | 13128 | AUUCACUGCUAGCAAGUCA | 13449 | UGACUUGCUAGCAGUGAAU |
| ETD02310 | 13233 | AUUCACUGCUAGCAAGUCA | 13554 | UGACUUGCUAGCAGUGAAU |
| ETD02311 | 13234 | AUUCACUGCUAGCAAGUCA | 13555 | UGACUUGCUAGCAGUGAAU |
| ETD02312 | 13235 | AUUCACUGCUAGCAAGUCA | 13556 | UGACUUGCUAGCAGUGAAU |
| ETD02313 | 13236 | AUUCACUGCUAGCAAGUCA | 13557 | UGACUUGCUAGCAGUGAAU |
| ETD02314 | 13237 | AUUCACUGCUAGCAAGUCA | 13558 | UGACUUGCUAGCAGUGAAU |
| ETD02315 | 13238 | AUUCACUGCUAGCAAGUCA | 13559 | UGACUUGCUAGCAGUGAAU |
| ETD02316 | 13239 | AUUCACUGCUAGCAAGUCA | 13560 | UGACUUGCUAGCAGUGAAU |
| ETD02317 | 13240 | AUUCACUGCUAGCAAGUCA | 13561 | UGACUUGCUAGCAGUGAAU |
| ETD02318 | 13241 | AUUCACUGCUAGCAAGUCA | 13562 | UGACUUGCUAGCAGUGAAU |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02009 | 13147 | UGGAAUCUCCUAUGAUCG | 13468 | CGAUCAUAGGAGAUUCCA |
| ETD02293 | 13242 | UGGAAUCUCCUAUGAUCG | 13563 | CGAUCAUAGGAGAUUCCA |
| ETD02294 | 13243 | UGGAAUCUCCUAUGAUCG | 13564 | CGAUCAUAGGAGAUUCCA |
| ETD02295 | 13244 | UGGAAUCUCCUAUGAUCG | 13565 | CGAUCAUAGGAGAUUCCA |
| ETD02296 | 13245 | UGGAAUCUCCUAUGAUCG | 13566 | CGAUCAUAGGAGAUUCCA |
| ETD02297 | 13246 | UGGAAUCUCCUAUGAUCG | 13567 | CGAUCAUAGGAGAUUCCA |
| ETD02298 | 13247 | UGGAAUCUCCUAUGAUCG | 13568 | CGAUCAUAGGAGAUUCCA |
| ETD02299 | 13248 | UGGAAUCUCCUAUGAUCG | 13569 | CGAUCAUAGGAGAUUCCA |
| ETD02300 | 13249 | UGGAAUCUCCUAUGAUCG | 13570 | CGAUCAUAGGAGAUUCCA |
| ETD02015 | 13153 | AUUCACUGCUAGCAAGUC | 13474 | GACUUGCUAGCAGUGAAU |
| ETD02310 | 13250 | AUUCACUGCUAGCAAGUC | 13571 | GACUUGCUAGCAGUGAAU |
| ETD02311 | 13251 | AUUCACUGCUAGCAAGUC | 13572 | GACUUGCUAGCAGUGAAU |
| ETD02312 | 13252 | AUUCACUGCUAGCAAGUC | 13573 | GACUUGCUAGCAGUGAAU |
| ETD02313 | 13253 | AUUCACUGCUAGCAAGUC | 13574 | GACUUGCUAGCAGUGAAU |
| ETD02314 | 13254 | AUUCACUGCUAGCAAGUC | 13575 | GACUUGCUAGCAGUGAAU |
| ETD02315 | 13255 | AUUCACUGCUAGCAAGUC | 13576 | GACUUGCUAGCAGUGAAU |
| ETD02316 | 13256 | AUUCACUGCUAGCAAGUC | 13577 | GACUUGCUAGCAGUGAAU |
| ETD02317 | 13257 | AUUCACUGCUAGCAAGUC | 13578 | GACUUGCUAGCAGUGAAU |
| ETD02318 | 13258 | AUUCACUGCUAGCAAGUC | 13579 | GACUUGCUAGCAGUGAAU |

TABLE 23
Relative GPAM mRNA Levels in Livers of
Mice Transfected with AAV8-TGB-h-GPAM

| | | | Dose | Mean human GPAM mRNA level |
|---|---|---|---|---|
| Group | n | Treatment | (ug) | (Normalized to Group 1, Day 14) |
| 1 | 5 | PBS | | 1.00 |
| 2 | 5 | ETD02009 | 100 | 1.46 |
| 3 | 5 | ETD02293 | 100 | 1.24 |
| 4 | 5 | ETD02294 | 100 | 0.61 |
| 5 | 5 | ETD02295 | 100 | 0.55 |
| 6 | 5 | ETD02296 | 100 | 0.42 |
| 7 | 5 | ETD02297 | 100 | 0.53 |
| 8 | 5 | ETD02298 | 100 | 0.70 |
| 9 | 5 | ETD02299 | 100 | 1.29 |
| 10 | 5 | ETD02300 | 100 | 0.42 |
| 11 | 5 | ETD02015 | 100 | 0.71 |
| 12 | 5 | ETD02310 | 100 | 0.21 |
| 13 | 5 | ETD02311 | 100 | 0.16 |
| 14 | 5 | ETD02312 | 100 | 0.15 |
| 15 | 5 | ETD02313 | 100 | 0.35 |
| 16 | 5 | ETD02314 | 100 | 0.29 |
| 17 | 5 | ETD02315 | 100 | 0.15 |
| 18 | 5 | ETD02316 | 100 | 0.33 |
| 19 | 5 | ETD02317 | 100 | 0.20 |
| 20 | 5 | ETD02318 | 100 | 0.08 |

### Example 15. Screening siRNAs with Alternative Modification Patterns of ETD02003 and ETD02018 in Mice Transfected with AAV8-TBG-h-GPAM

The base sequences of ETD02003 and ETD02018 were synthesized to generate siRNAs (ETD02223-ETD02230, and ETD02201-ETD02208, respectively) with alternative modification patterns. The activities of the siRNAs were assessed using mice transiently expressing human GPAM. The siRNAs contain the GalNAc ligand ETL17 followed by a phosphorothioate linkage at the 5′ end of the sense strand. The siRNA sequences that were used are shown in Table 24, where Nf (e.g. Af, Cf, Gf, Tf, or Uf) is a 2′ fluoro-modified nucleoside, n (e.g. a, c, g, t, or u) is a 2′ O-methyl modified nucleoside, dN (e.g. dA, dC, dG, dT, or dU) is a 2′ deoxynucleoside, and “s” is a phosphorothioate linkage. The base sequences for each siRNA, with and without the 3′ UU extension, are shown in Table 25.

Six to eight week old female mice (C57Bl/6) were injected with 5 uL of a recombinant adeno-associated virus 8 (AAV8) vector (1.3×10E13 genome copies/mL) by the retroorbital route on Day −14. The recombinant AAV8 contains the open reading frame, the 5′ UTR, and a portion of the 3′UTR of the human GPAM sequence (ENST00000348367) under the control of the human thyroxine binding globulin promoter in an AAV2 backbone packaged in AAV8 capsid (AAV8-TBG-h-GPAM). On Day 0, infected mice were given a subcutaneous injection of a single 100 ug dose of a GalNAc-conjugated siRNA or PBS as vehicle control (n=4).

Mice were euthanized on Day 14 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver GPAM mRNA were assessed by RT-qPCR in triplicate on a QuantStudio™ 6 Pro Real-Time PCR System using TaqMan assays for human GPAM (ThermoFisher, assay #Hs01573684_m1) or mouse GPAM (ThermoFisher, assay #Mm01261106_m1), and the mouse housekeeping gene PPIA (ThermoFisher, assay #Mm02342430_g1), and PerfeCTa® qPCR FastMix®, Low ROX™ (VWR, Catalog #101419-222). Data were normalized to the mean GPAM mRNA level in animals receiving a subcutaneous injection of PBS. Results are shown in Table 26. Of the alternatively modified versions of ETD02003, ETD02224 and ETD02228 had the greatest activity in terms of knockdown of the human GPAM mRNA. Of the alternatively modified versions of ETD02018, ETD02202 had the greatest activity in terms of knockdown of the human GPAM mRNA.

TABLE 24
Example siRNA Sequences

| | | | SEQ | |
|---|---|---|---|---|
| | SEQ | Sense Strand Sequence (5′-3′) with | ID | |
| ETD# | ID NO: | GalNAc moiety | NO: | Antisense Strand Sequence (5′-3′) |
| ETD02223 | 12927 | [ETL17]succaUfgdGgCfaUfauaguugasusu | 13034 | usCfsaAfcUfaUfaUfgCfcCfaUfgGfasusu |
| ETD02224 | 12928 | [ETL17]succaUfgggCfaUfauaguugasusu | 13035 | usCfsaacUfaUfaUfgCfcCfaUfgGfasusu |
| ETD02225 | 12929 | [ETL17]succaUfgggCfaUfauaguugasusu | 13036 | usCfsaacUfaUfaUfgccCfaUfgGfasusu |
| ETD02226 | 12930 | [ETL17]succaUfgggCfaUfauaguugasusu | 13037 | usCfsaAfcUfaUfaUfgccCfaUfgGfasusu |
| ETD02227 | 12931 | [ETL17]succaUfgggCfaUfauaguugasusu | 13038 | usCfsaAfcuAfuaUfgCfcCfaUfgGfasusu |
| ETD02228 | 12932 | [ETL17]succaUfgggCfaUfauaguugasusu | 13039 | usCfsaAfcuAfuaUfgccCfaUfgGfasusu |
| ETD02229 | 12933 | [ETL17]succaUfgggCfaUfauaguugasusu | 13040 | usCfsaaCfuAfuAfuGfccCfaUfgGfasusu |
| ETD02230 | 12934 | [ETL17]succaUfgggCfaUfauaguugasusu | 13041 | usCfsaAfcUfaUfaUfgCfcCfaUfggasusu |
| ETD02201 | 12935 | [ETL17]sauauaUfUfCfUfgaguuuuguasusu | 13042 | usAfscaaAfaCfuCfaGfaAfuAfuAfususu |
| ETD02202 | 12936 | [ETL17]sauauaUfUfCfUfgaguuuuguasusu | 13043 | usAfscaaAfaCfuCfagaAfuAfuAfususu |
| ETD02203 | 12937 | [ETL17]sauauaUfUfCfUfgaguuuuguasusu | 13044 | usAfscAfaAfaCfuCfagaAfuAfuAfususu |
| ETD02204 | 12938 | [ETL17]sauauaUfUfCfUfgaguuuuguasusu | 13045 | usAfscAfaAfaCfucaGfaAfuAfuAfususu |
| ETD02205 | 12939 | [ETL17]sauauaUfUfCfUfgaguuuuguasusu | 13046 | usAfscAfaaAfcucaGfaAfuAfuAfususu |
| ETD02206 | 12940 | [ETL17]sauauaUfUfCfUfgaguuuuguasusu | 13047 | usAfscAfaaAfcUfcaGfaAfuAfuAfususu |
| ETD02207 | 12941 | [ETL17]sauauaUfUfCfUfgaguuuuguasusu | 13048 | usAfscAfaaAfcuCfagaAfuAfuAfususu |
| ETD02208 | 12942 | [ETL17]sauauaUfUfCfUfgaguuuuguasusu | 13049 | usAfscaAfaAfcuCfagaAfuAfuAfususu |

TABLE 25
Example siRNA BASE Sequences

| siRNA | SEQ ID | Sense Strand Base Sequence | SEQ ID | Antisense Strand Base |
|---|---|---|---|---|
| Name | NO: | (5′ to 3′) | NO | Sequence (5′ to 3′) |
| ETD02223 | 13259 | UCCAUGGGCAUAUAGUUGAUU | 13580 | UCAACUAUAUGCCCAUGGAUU |
| ETD02224 | 13260 | UCCAUGGGCAUAUAGUUGAUU | 13581 | UCAACUAUAUGCCCAUGGAUU |
| ETD02225 | 13261 | UCCAUGGGCAUAUAGUUGAUU | 13582 | UCAACUAUAUGCCCAUGGAUU |
| ETD02226 | 13262 | UCCAUGGGCAUAUAGUUGAUU | 13583 | UCAACUAUAUGCCCAUGGAUU |
| ETD02227 | 13263 | UCCAUGGGCAUAUAGUUGAUU | 13584 | UCAACUAUAUGCCCAUGGAUU |
| ETD02228 | 13264 | UCCAUGGGCAUAUAGUUGAUU | 13585 | UCAACUAUAUGCCCAUGGAUU |
| ETD02229 | 13265 | UCCAUGGGCAUAUAGUUGAUU | 13586 | UCAACUAUAUGCCCAUGGAUU |
| ETD02230 | 13266 | UCCAUGGGCAUAUAGUUGAUU | 13587 | UCAACUAUAUGCCCAUGGAUU |
| ETD02201 | 13267 | AUAUAUUCUGAGUUUUGUAUU | 13588 | UACAAAACUCAGAAUAUAUUU |
| ETD02202 | 13268 | AUAUAUUCUGAGUUUUGUAUU | 13589 | UACAAAACUCAGAAUAUAUUU |
| ETD02203 | 13269 | AUAUAUUCUGAGUUUUGUAUU | 13590 | UACAAAACUCAGAAUAUAUUU |
| ETD02204 | 13270 | AUAUAUUCUGAGUUUUGUAUU | 13591 | UACAAAACUCAGAAUAUAUUU |
| ETD02205 | 13271 | AUAUAUUCUGAGUUUUGUAUU | 13592 | UACAAAACUCAGAAUAUAUUU |
| ETD02206 | 13272 | AUAUAUUCUGAGUUUUGUAUU | 13593 | UACAAAACUCAGAAUAUAUUU |
| ETD02207 | 13273 | AUAUAUUCUGAGUUUUGUAUU | 13594 | UACAAAACUCAGAAUAUAUUU |
| ETD02208 | 13274 | AUAUAUUCUGAGUUUUGUAUU | 13595 | UACAAAACUCAGAAUAUAUUU |
| | | Sense Strand Base | | Antisense Strand Base |
| siRNA | SEQ ID | Sequence (5′ to 3′), | SEQ ID | Sequence (5′ to 3′), |
| Name | NO: | without 3′ overhangs | NO | without 3′ overhangs |
| ETD02223 | 13275 | UCCAUGGGCAUAUAGUUGA | 13596 | UCAACUAUAUGCCCAUGGA |
| ETD02224 | 13276 | UCCAUGGGCAUAUAGUUGA | 13597 | UCAACUAUAUGCCCAUGGA |
| ETD02225 | 13277 | UCCAUGGGCAUAUAGUUGA | 13598 | UCAACUAUAUGCCCAUGGA |
| ETD02226 | 13278 | UCCAUGGGCAUAUAGUUGA | 13599 | UCAACUAUAUGCCCAUGGA |
| ETD02227 | 13279 | UCCAUGGGCAUAUAGUUGA | 13600 | UCAACUAUAUGCCCAUGGA |
| ETD02228 | 13280 | UCCAUGGGCAUAUAGUUGA | 13601 | UCAACUAUAUGCCCAUGGA |
| ETD02229 | 13281 | UCCAUGGGCAUAUAGUUGA | 13602 | UCAACUAUAUGCCCAUGGA |
| ETD02230 | 13282 | UCCAUGGGCAUAUAGUUGA | 13603 | UCAACUAUAUGCCCAUGGA |
| ETD02201 | 13283 | AUAUAUUCUGAGUUUUGUA | 13604 | UACAAAACUCAGAAUAUAU |
| ETD02202 | 13284 | AUAUAUUCUGAGUUUUGUA | 13605 | UACAAAACUCAGAAUAUAU |
| ETD02203 | 13285 | AUAUAUUCUGAGUUUUGUA | 13606 | UACAAAACUCAGAAUAUAU |
| ETD02204 | 13286 | AUAUAUUCUGAGUUUUGUA | 13607 | UACAAAACUCAGAAUAUAU |
| ETD02205 | 13287 | AUAUAUUCUGAGUUUUGUA | 13608 | UACAAAACUCAGAAUAUAU |
| ETD02206 | 13288 | AUAUAUUCUGAGUUUUGUA | 13609 | UACAAAACUCAGAAUAUAU |
| ETD02207 | 13289 | AUAUAUUCUGAGUUUUGUA | 13610 | UACAAAACUCAGAAUAUAU |
| ETD02208 | 13290 | AUAUAUUCUGAGUUUUGUA | 13611 | UACAAAACUCAGAAUAUAU |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02223 | 13291 | UCCAUGGGCAUAUAGUUG | 13612 | CAACUAUAUGCCCAUGGA |
| ETD02224 | 13292 | UCCAUGGGCAUAUAGUUG | 13613 | CAACUAUAUGCCCAUGGA |
| ETD02225 | 13293 | UCCAUGGGCAUAUAGUUG | 13614 | CAACUAUAUGCCCAUGGA |
| ETD02226 | 13294 | UCCAUGGGCAUAUAGUUG | 13615 | CAACUAUAUGCCCAUGGA |
| ETD02227 | 13295 | UCCAUGGGCAUAUAGUUG | 13616 | CAACUAUAUGCCCAUGGA |
| ETD02228 | 13296 | UCCAUGGGCAUAUAGUUG | 13617 | CAACUAUAUGCCCAUGGA |
| ETD02229 | 13297 | UCCAUGGGCAUAUAGUUG | 13618 | CAACUAUAUGCCCAUGGA |
| ETD02230 | 13298 | UCCAUGGGCAUAUAGUUG | 13619 | CAACUAUAUGCCCAUGGA |
| ETD02201 | 13299 | AUAUAUUCUGAGUUUUGU | 13620 | ACAAAACUCAGAAUAUAU |
| ETD02202 | 13300 | AUAUAUUCUGAGUUUUGU | 13621 | ACAAAACUCAGAAUAUAU |
| ETD02203 | 13301 | AUAUAUUCUGAGUUUUGU | 13622 | ACAAAACUCAGAAUAUAU |
| ETD02204 | 13302 | AUAUAUUCUGAGUUUUGU | 13623 | ACAAAACUCAGAAUAUAU |
| ETD02205 | 13303 | AUAUAUUCUGAGUUUUGU | 13624 | ACAAAACUCAGAAUAUAU |
| ETD02206 | 13304 | AUAUAUUCUGAGUUUUGU | 13625 | ACAAAACUCAGAAUAUAU |
| ETD02207 | 13305 | AUAUAUUCUGAGUUUUGU | 13626 | ACAAAACUCAGAAUAUAU |
| ETD02208 | 13306 | AUAUAUUCUGAGUUUUGU | 13627 | ACAAAACUCAGAAUAUAU |

TABLE 26
Relative GPAM mRNA Levels in Livers of
Mice Transfected with AAV8-TGB-h-GPAM

| | | | | Mean GPAM mRNA | |
|---|---|---|---|---|---|
| | | | | (Normalized to Group 1, Day 14) | |
| | | | Dose | Human | Mouse |
| Group | n | Treatment | (ug) | GPAM mRNA | GPAM mRNA |
| 1 | 4 | PBS | | 1.00 | 1.00 |
| 2 | 4 | ETD02223 | 100 | 0.54 | 0.52 |
| 3 | 4 | ETD02224 | 100 | 0.17 | 0.10 |
| 4 | 4 | ETD02225 | 100 | 1.25 | 0.88 |
| 5 | 4 | ETD02226 | 100 | 0.35 | 0.12 |
| 6 | 4 | ETD02227 | 100 | 0.19 | 0.16 |
| 7 | 4 | ETD02228 | 100 | 0.11 | 0.16 |
| 8 | 4 | ETD02229 | 100 | 0.34 | 0.10 |
| 9 | 4 | ETD02230 | 100 | 0.29 | 0.08 |
| 10 | 4 | ETD02201 | 100 | 0.44 | 0.04 |
| 11 | 4 | ETD02202 | 100 | 0.29 | 0.06 |
| 12 | 4 | ETD02203 | 100 | 0.78 | 0.08 |
| 13 | 4 | ETD02204 | 100 | 0.57 | 0.11 |
| 14 | 4 | ETD02205 | 100 | 0.60 | 0.12 |
| 15 | 4 | ETD02206 | 100 | 0.44 | 0.07 |
| 16 | 4 | ETD02207 | 100 | 0.55 | 0.08 |
| 17 | 4 | ETD02208 | 100 | 1.03 | 0.07 |

### Example 16. Screening Modification Patterns of ETD02009 and ETD02015 Containing 2′-O-(2-Methoxyethyl) in Mice Transfected with AAV8-TBG-h-GPAM

The base sequences of ETD02009 and ETD02015 were synthesized to generate siRNAs (ETD02543-ETD02544, and ETD02539-ETD02542, respectively) with alternative modification patterns including 2′-O-(2-methoxyethyl). The activities of the siRNAs were assessed using mice transiently expressing human GPAM. The siRNAs contain the GalNAc ligand ETL17 followed by a phosphorothioate linkage at the 5′ end of the sense strand. The siRNA sequences that were used are shown in Table 27, where Nf (e.g. Af, Cf, Gf, Tf, or Uf) is a 2′ fluoro-modified nucleoside, n (e.g. a, c, g, t, or u) is a 2′ O-methyl modified nucleoside, Nm (e.g. Am, Cm, Gm, Tm, or Um) is a 2′-O-(2-methoxyethyl) modified nucleoside, and “s” is a phosphorothioate linkage. The base sequences for each siRNA, with and without the 3′ UU extension, are shown in Table 28.

Six to eight week old female mice (C57Bl/6) were injected with 5 uL of a recombinant adeno-associated virus 8 (AAV8) vector (1.2×10E13 genome copies/mL) by the retroorbital route on Day −14. The recombinant AAV8 contains the open reading frame, the 5′ UTR, and a portion of the 3′UTR of the human GPAM sequence (ENST00000348367) under the control of the human thyroxine binding globulin promoter in an AAV2 backbone packaged in AAV8 capsid (AAV8-TBG-h-GPAM). On Day 0, infected mice were given a subcutaneous injection of a single 100 ug dose of a GalNAc-conjugated siRNA or PBS as vehicle control (n=5).

Mice were euthanized on Day 14 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver GPAM mRNA were assessed by RT-qPCR in triplicate on a QuantStudio™ 6 Pro Real-Time PCR System using TaqMan assays for human GPAM (ThermoFisher, assay #Hs00326039_m1) and the mouse housekeeping gene PPIA (ThermoFisher, assay #Mm02342430_g1), and PerfeCTa® qPCR FastMix®, Low ROX™ (VWR, Catalog #101419-222). Data were normalized to the mean GPAM mRNA level in animals receiving a subcutaneous injection of PBS. Results are shown in Table 29. Of the alternatively modified versions of ETD02015, ETD02541 had the greatest activity. Of the alternatively modified versions of ETD02009, ETD02543 and ETD2544 had the greatest activity.

TABLE 27
Example siRNA Sequences

| | SEQ | | SEQ | |
|---|---|---|---|---|
| | ID | Sense Strand Sequence (5′-3′) with GalNAc | ID | |
| ETD# | NO | moiety | NO | Antisense Strand Sequence (5′-3′) |
| ETD02015 | 12889 | [ETL17]sauucaCfUfgCfUfagcaagucasusu | 12996 | usGfsaCfuUfgCfuAfgCfaGfuGfaAfususu |
| ETD02009 | 12883 | [ETL17]suggaaUfCfuCfCfuaugaucgasusu | 12990 | usCfsgAfuCfaUfaGfgAfgAfuUfcCfasusu |
| ETD02539 | 12943 | [ETL17]sauu CmaCfUfGfCfuagCmaagucasusu | 13050 | usGfsacuUfgCfuAfgCfaGfuGfaAfususu |
| ETD02540 | 12944 | [ETL17]sauuCmaCfUfGfCfuaGmcaagucasusu | 13051 | usGfsacuUfgCfuAfgCfaGfuGfaAfususu |
| ETD02541 | 12945 | [ETL17]sauuCmaCfUfGfCfuaGmcaagucasusu | 13052 | usGfsacUfuGfcUfagcaGfuGfaAfususu |
| ETD02542 | 12946 | [ETL17]sauuCmaCfUfGfCfuagCmaagucasusu | 13053 | usGfsacUfuGfcUfagcaGfuGfaAfususu |
| ETD02543 | 12947 | [ETL17]suggAmaUfCfUfCfcuAmugaucgasusu | 13054 | usCfsgAfucAfuAfggagAfuUfcCfasusu |
| ETD02544 | 12948 | [ETL17]suggAmaUfCfUfCfcuaTmgaucgasusu | 13055 | usCfsgAfucAfuAfggagAfuUfcCfasusu |

TABLE 28
Example siRNA BASE Sequences

| siRNA | SEQ ID | Sense Strand Base Sequence | SEQ ID | Antisense Strand Base |
|---|---|---|---|---|
| Name | NO: | (5′ to 3′) | NO | Sequence (5′ to 3′) |
| ETD02015 | 13103 | AUUCACUGCUAGCAAGUCAUU | 13424 | UGACUUGCUAGCAGUGAAUUU |
| ETD02009 | 13097 | UGGAAUCUCCUAUGAUCGAUU | 13418 | UCGAUCAUAGGAGAUUCCAUU |
| ETD02539 | 13307 | AUUCACUGCUAGCAAGUCAUU | 13628 | UGACUUGCUAGCAGUGAAUUU |
| ETD02540 | 13308 | AUUCACUGCUAGCAAGUCAUU | 13629 | UGACUUGCUAGCAGUGAAUUU |
| ETD02541 | 13309 | AUUCACUGCUAGCAAGUCAUU | 13630 | UGACUUGCUAGCAGUGAAUUU |
| ETD02542 | 13310 | AUUCACUGCUAGCAAGUCAUU | 13631 | UGACUUGCUAGCAGUGAAUUU |
| ETD02543 | 13311 | UGGAAUCUCCUAUGAUCGAUU | 13632 | UCGAUCAUAGGAGAUUCCAUU |
| ETD02544 | 13312 | UGGAAUCUCCUAUGAUCGAUU | 13633 | UCGAUCAUAGGAGAUUCCAUU |
| | | Sense Strand Base | | Antisense Strand Base |
| siRNA | SEQ ID | Sequence (5′ to 3′), | SEQ ID | Sequence (5′ to 3′), |
| Name | NO: | without 3′ overhangs | NO | without 3′ overhangs |
| ETD02015 | 13128 | AUUCACUGCUAGCAAGUCA | 13449 | UGACUUGCUAGCAGUGAAU |
| ETD02009 | 13122 | UGGAAUCUCCUAUGAUCGA | 13443 | UCGAUCAUAGGAGAUUCCA |
| ETD02539 | 13313 | AUUCACUGCUAGCAAGUCA | 13634 | UGACUUGCUAGCAGUGAAU |
| ETD02540 | 13314 | AUUCACUGCUAGCAAGUCA | 13635 | UGACUUGCUAGCAGUGAAU |
| ETD02541 | 13315 | AUUCACUGCUAGCAAGUCA | 13636 | UGACUUGCUAGCAGUGAAU |
| ETD02542 | 13316 | AUUCACUGCUAGCAAGUCA | 13637 | UGACUUGCUAGCAGUGAAU |
| ETD02543 | 13317 | UGGAAUCUCCUAUGAUCGA | 13638 | UCGAUCAUAGGAGAUUCCA |
| ETD02544 | 13318 | UGGAAUCUCCUAUGAUCGA | 13639 | UCGAUCAUAGGAGAUUCCA |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02015 | 13153 | AUUCACUGCUAGCAAGUC | 13474 | GACUUGCUAGCAGUGAAU |
| ETD02009 | 13147 | UGGAAUCUCCUAUGAUCG | 13468 | CGAUCAUAGGAGAUUCCA |
| ETD02539 | 13319 | AUUCACUGCUAGCAAGUC | 13640 | GACUUGCUAGCAGUGAAU |
| ETD02540 | 13320 | AUUCACUGCUAGCAAGUC | 13641 | GACUUGCUAGCAGUGAAU |
| ETD02541 | 13321 | AUUCACUGCUAGCAAGUC | 13642 | GACUUGCUAGCAGUGAAU |
| ETD02542 | 13322 | AUUCACUGCUAGCAAGUC | 13643 | GACUUGCUAGCAGUGAAU |
| ETD02543 | 13323 | UGGAAUCUCCUAUGAUCG | 13644 | CGAUCAUAGGAGAUUCCA |
| ETD02544 | 13324 | UGGAAUCUCCUAUGAUCG | 13645 | CGAUCAUAGGAGAUUCCA |

TABLE 29
Relative GPAM mRNA Levels in Livers of
Mice Transfected with AAV8-TGB-h-GPAM

| | | | Dose | Mean human GPAM mRNA level |
|---|---|---|---|---|
| Group | n | Treatment | (ug) | (Normalized to Group 1, Day 14) |
| 1 | 5 | PBS | | 1.00 |
| 2 | 5 | ETD02015 | 100 | 1.25 |
| 3 | 5 | ETD02539 | 100 | 1.27 |
| 4 | 5 | ETD02540 | 100 | 0.84 |
| 5 | 5 | ETD02541 | 100 | 0.79 |
| 6 | 5 | ETD02542 | 100 | 0.88 |
| 7 | 5 | ETD02009 | 100 | 1.64 |
| 8 | 5 | ETD02543 | 100 | 0.33 |
| 9 | 5 | ETD02544 | 100 | 0.43 |

### Example 17. Screening of siRNAs ETD02553-ETD02576 Targeting Human GPAM mRNA in Mice Transfected with AAV8-TBG-h-GPAM

The activities of siRNAs, namely ETD02553-ETD02576, were assessed in mice transiently expressing human GPAM. The siRNAs contain the GalNAc ligand ETL17 followed by a phosphorothioate linkage at the 5′ end of the sense strand. The siRNA sequences that were used are shown in Table 30, where Nf (e.g. Af, Cf, Gf, Tf, or Uf) is a 2′ fluoro-modified nucleoside, n (e.g. a, c, g, t, or u) is a 2′ O-methyl modified nucleoside, Nm (e.g. Am, Cm, Gm, Tm, or Um) is a 2′-O-(2-methoxyethyl) modified nucleoside, and “s” is a phosphorothioate linkage. The base sequences for each siRNA, with and without the 3′ UU extension, are shown in Table 31. ETD02553-ETD02664 were tested in Part 1 of the study and ETD02565-ETD02576 were tested in Part 2.

Six to eight week old female mice (C57Bl/6) were injected with 10 uL of a recombinant adeno-associated virus 8 (AAV8) vector (1.3×10E13 genome copies/mL) by the retroorbital route on Day −14. The recombinant AAV8 contains the open reading frame, the 5′ UTR, and a portion of the 3′UTR of the human GPAM sequence (ENST00000348367) under the control of the human thyroxine binding globulin promoter in an AAV2 backbone packaged in AAV8 capsid (AAV8-TBG-h-GPAM). On Day 0, infected mice were given a subcutaneous injection of a single 100 ug dose of a GalNAc-conjugated siRNA or PBS as vehicle control (n=5).

Mice were euthanized on Day 14 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver GPAM mRNA were assessed by RT-qPCR in triplicate on a QuantStudio™ 6 Pro Real-Time PCR System using TaqMan assays for human GPAM (ThermoFisher, assay #Hs01573684_m1) and the mouse housekeeping gene PPIA (ThermoFisher, assay #Mm02342430_g1), and PerfeCTa® qPCR FastMix®, Low ROX™ (VWR, Catalog #101419-222). Mice that gave low liver expression of human GPAM in Part 2, as defined by having a Ct value of >32, were omitted from further analysis. Data were normalized to the mean GPAM mRNA level in animals receiving a subcutaneous injection of PBS. Results for Part 1 are shown in Table 32, and those for Part 2 in Table 33. Of the siRNAs in this screening set, mice injected with ETD02557, ETD02562, ETD02563, ETD02564, ETD02572, ETD02573 or ETD02574 had the highest level of human GPAM mRNA knockdown in the liver.

TABLE 30
Example siRNA Sequences

| | SEQ | | SEQ | |
|---|---|---|---|---|
| | ID | Sense Strand Sequence (5′-3′) with | ID | |
| ETD# | NO: | GalNAc moiety | NO: | Antisense Strand Sequence (5′-3′) |
| ETD02553 | 12949 | [ETL17]saaaCmuGfAfUfAfgcuGmaguccasusu | 13056 | usGfsgAfcUfcAfgCfuAfuCfaGfuUfususu |
| ETD02554 | 12950 | [ETL17]suugTmgAfCfAfUfggaTmgaaucasusu | 13057 | usGfsaUfuCfaUfcCfaUfgUfcAfcAfasusu |
| ETD02555 | 12951 | [ETL17]sagaAmuAfCfAfGfuguTmggucgasusu | 13058 | usCfsgAfcCfaAfcAfcUfgUfaUfuCfususu |
| ETD02556 | 12952 | [ETL17]saauAmcAfGfUfGfuugGmucgauasusu | 13059 | usAfsuCfgAfcCfaAfcAfcUfgUfaUfususu |
| ETD02557 | 12953 | [ETL17]sauaCmaGfUfGfUfuggTmegaugasusu | 13060 | usCfsaUfcGfaCfcAfaCfaCfuGfuAfususu |
| ETD02558 | 12954 | [ETL17]suacAmgUfGfUfUfgguCmgauguasusu | 13061 | usAfscAfuCfgAfcCfaAfcAfcUfgUfasusu |
| ETD02559 | 12955 | [ETL17]saugTmaAfGfCfAfcacAmagugaasusu | 13062 | usUfscAfcUfuGfuGfuGfcUfuAfcAfususu |
| ETD02560 | 12956 | [ETL17]suuuTmuCfAfAfCfcccAmguaucasusu | 13063 | usGfsaUfaCfuGfgGfgUfuGfaAfaAfasusu |
| ETD02561 | 12957 | [ETL17]scuuTmuUfAfUfUfcaaGmagegaasusu | 13064 | usUfscGfcUfcUfuGfaAfuAfaAfaAfgsusu |
| ETD02562 | 12958 | [ETL17]suauTmcAfAfGfAfgcgAmgauguasusu | 13065 | usAfscAfuCfuCfgCfuCfuUfgAfaUfasusu |
| ETD02563 | 12959 | [ETL17]scauTmgCfUfUfCfaggCmaauaaasusu | 13066 | usUfsuAfuUfgCfcUfgAfaGfcAfaUfgsusu |
| ETD02564 | 12960 | [ETL17]saauTmaCfUfUfCfgacAmgcagcasusu | 13067 | usGfscUfgCfuGfuCfgAfaGfuAfaUfususu |
| ETD02565 | 12961 | [ETL17]sauuAmcUfUfCfGfacaGmcagcaasusu | 13068 | usUfsgCfuGfcUfgUfcGfaAfgUfaAfususu |
| ETD02566 | 12962 | [ETL17]scuuTmuGfUfCfAfguuGmugguaasusu | 13069 | usUfsaCfcAfcAfaCfuGfaCfaAfaAfgsusu |
| ETD02567 | 12963 | [ETL17]scuuGmaUfAfAfUfaccTmguuggasusu | 13070 | usCfscAfaCfaGfgUfaUfuAfuCfaAfgsusu |
| ETD02568 | 12964 | [ETL17]sugaTmaAfUfAfCfcugTmuggaaasusu | 13071 | usUfsuCfcAfaCfaGfgUfaUfuAfuCfasusu |
| ETD02569 | 12965 | [ETL17]saagAmgGfUfGfUfuauTmagaauasusu | 13072 | usAfsuUfcUfaAfuAfaCfaCfcUfcUfususu |
| ETD02570 | 12966 | [ETL17]suguTmaCfGfAfAfaaaAmcuaugasusu | 13073 | usCfsaUfaGfuUfuUfuUfcGfuAfaCfasusu |
| ETD02571 | 12967 | [ETL17]saucTmgGfCfUfGfagcAmuauucasusu | 13074 | usGfsaAfuAfuGfcUfcAfgCfcAfgAfususu |
| ETD02572 | 12968 | [ETL17]scuaTmuCfAfCfUfgcuAmgcaagasusu | 13075 | usCfsuUfgCfuAfgCfaGfuGfaAfuAfgsusu |
| ETD02573 | 12969 | [ETL17]suagTmaAfUfGfCfaugCmcauacasusu | 13076 | usGfsuAfuGfgCfaUfgCfaUfuAfcUfasusu |
| ETD02574 | 12970 | [ETL17]saauGmgGfGfUfAfcuuCmaugucasusu | 13077 | usGfsaCfaUfgAfaGfuAfcCfcCfaUfususu |
| ETD02575 | 12971 | [ETL17]saggAmaAfGfUfUfuauCmcaguaasusu | 13078 | usUfsaCfuGfgAfuAfaAfcUfuUfcCfususu |
| ETD02576 | 12972 | [ETL17]sucuGmcAfAfAfAfguuGmcacaaasusu | 13079 | usUfsuGfuGfcAfaCfuUfuUfgCfaGfasusu |

TABLE 31
Example siRNA BASE Sequences

| siRNA | SEQ ID | Sense Strand Base Sequence | SEQ ID | Antisense Strand Base |
|---|---|---|---|---|
| Name | NO: | (5′ to 3′) | NO | Sequence (5′ to 3′) |
| ETD02553 | 13325 | AAACUGAUAGCUGAGUCCAUU | 13646 | UGGACUCAGCUAUCAGUUUUU |
| ETD02554 | 13326 | UUGUGACAUGGAUGAAUCAUU | 13647 | UGAUUCAUCCAUGUCACAAUU |
| ETD02555 | 13327 | AGAAUACAGUGUUGGUCGAUU | 13648 | UCGACCAACACUGUAUUCUUU |
| ETD02556 | 13328 | AAUACAGUGUUGGUCGAUAUU | 13649 | UAUCGACCAACACUGUAUUUU |
| ETD02557 | 13329 | AUACAGUGUUGGUCGAUGAUU | 13650 | UCAUCGACCAACACUGUAUUU |
| ETD02558 | 13330 | UACAGUGUUGGUCGAUGUAUU | 13651 | UACAUCGACCAACACUGUAUU |
| ETD02559 | 13331 | AUGUAAGCACACAAGUGAAUU | 13652 | UUCACUUGUGUGCUUACAUUU |
| ETD02560 | 13332 | UUUUUCAACCCCAGUAUCAUU | 13653 | UGAUACUGGGGUUGAAAAAUU |
| ETD02561 | 13333 | CUUUUUAUUCAAGAGCGAAUU | 13654 | UUCGCUCUUGAAUAAAAAGUU |
| ETD02562 | 13334 | UAUUCAAGAGCGAGAUGUAUU | 13655 | UACAUCUCGCUCUUGAAUAUU |
| ETD02563 | 13335 | CAUUGCUUCAGGCAAUAAAUU | 13656 | UUUAUUGCCUGAAGCAAUGUU |
| ETD02564 | 13336 | AAUUACUUCGACAGCAGCAUU | 13657 | UGCUGCUGUCGAAGUAAUUUU |
| ETD02565 | 13337 | AUUACUUCGACAGCAGCAAUU | 13658 | UUGCUGCUGUCGAAGUAAUUU |
| ETD02566 | 13338 | CUUUUGUCAGUUGUGGUAAUU | 13659 | UUACCACAACUGACAAAAGUU |
| ETD02567 | 13339 | CUUGAUAAUACCUGUUGGAUU | 13660 | UCCAACAGGUAUUAUCAAGUU |
| ETD02568 | 13340 | UGAUAAUACCUGUUGGAAAUU | 13661 | UUUCCAACAGGUAUUAUCAUU |
| ETD02569 | 13341 | AAGAGGUGUUAUUAGAAUAUU | 13662 | UAUUCUAAUAACACCUCUUUU |
| ETD02570 | 13342 | UGUUACGAAAAAACUAUGAUU | 13663 | UCAUAGUUUUUUCGUAACAUU |
| ETD02571 | 13343 | AUCUGGCUGAGCAUAUUCAUU | 13664 | UGAAUAUGCUCAGCCAGAUUU |
| ETD02572 | 13344 | CUAUUCACUGCUAGCAAGAUU | 13665 | UCUUGCUAGCAGUGAAUAGUU |
| ETD02573 | 13345 | UAGUAAUGCAUGCCAUACAUU | 13666 | UGUAUGGCAUGCAUUACUAUU |
| ETD02574 | 13346 | AAUGGGGUACUUCAUGUCAUU | 13667 | UGACAUGAAGUACCCCAUUUU |
| ETD02575 | 13347 | AGGAAAGUUUAUCCAGUAAUU | 13668 | UUACUGGAUAAACUUUCCUUU |
| ETD02576 | 13348 | UCUGCAAAAGUUGCACAAAUU | 13669 | UUUGUGCAACUUUUGCAGAUU |
| | | Sense Strand Base | | Antisense Strand Base |
| siRNA | SEQ ID | Sequence (5′ to 3′), | SEQ ID | Sequence (5′ to 3′), |
| Name | NO″ | without 3′ overhangs | NO | without 3′ overhangs |
| ETD02553 | 13349 | AAACUGAUAGCUGAGUCCA | 13670 | UGGACUCAGCUAUCAGUUU |
| ETD02554 | 13350 | UUGUGACAUGGAUGAAUCA | 13671 | UGAUUCAUCCAUGUCACAA |
| ETD02555 | 13351 | AGAAUACAGUGUUGGUCGA | 13672 | UCGACCAACACUGUAUUCU |
| ETD02556 | 13352 | AAUACAGUGUUGGUCGAUA | 13673 | UAUCGACCAACACUGUAUU |
| ETD02557 | 13353 | AUACAGUGUUGGUCGAUGA | 13674 | UCAUCGACCAACACUGUAU |
| ETD02558 | 13354 | UACAGUGUUGGUCGAUGUA | 13675 | UACAUCGACCAACACUGUA |
| ETD02559 | 13355 | AUGUAAGCACACAAGUGAA | 13676 | UUCACUUGUGUGCUUACAU |
| ETD02560 | 13356 | UUUUUCAACCCCAGUAUCA | 13677 | UGAUACUGGGGUUGAAAAA |
| ETD02561 | 13357 | CUUUUUAUUCAAGAGCGAA | 13678 | UUCGCUCUUGAAUAAAAAG |
| ETD02562 | 13358 | UAUUCAAGAGCGAGAUGUA | 13679 | UACAUCUCGCUCUUGAAUA |
| ETD02563 | 13359 | CAUUGCUUCAGGCAAUAAA | 13680 | UUUAUUGCCUGAAGCAAUG |
| ETD02564 | 13360 | AAUUACUUCGACAGCAGCA | 13681 | UGCUGCUGUCGAAGUAAUU |
| ETD02565 | 13361 | AUUACUUCGACAGCAGCAA | 13682 | UUGCUGCUGUCGAAGUAAU |
| ETD02566 | 13362 | CUUUUGUCAGUUGUGGUAA | 13683 | UUACCACAACUGACAAAAG |
| ETD02567 | 13363 | CUUGAUAAUACCUGUUGGA | 13684 | UCCAACAGGUAUUAUCAAG |
| ETD02568 | 13364 | UGAUAAUACCUGUUGGAAA | 13685 | UUUCCAACAGGUAUUAUCA |
| ETD02569 | 13365 | AAGAGGUGUUAUUAGAAUA | 13686 | UAUUCUAAUAACACCUCUU |
| ETD02570 | 13366 | UGUUACGAAAAAACUAUGA | 13687 | UCAUAGUUUUUUCGUAACA |
| ETD02571 | 13367 | AUCUGGCUGAGCAUAUUCA | 13688 | UGAAUAUGCUCAGCCAGAU |
| ETD02572 | 13368 | CUAUUCACUGCUAGCAAGA | 13689 | UCUUGCUAGCAGUGAAUAG |
| ETD02573 | 13369 | UAGUAAUGCAUGCCAUACA | 13690 | UGUAUGGCAUGCAUUACUA |
| ETD02574 | 13370 | AAUGGGGUACUUCAUGUCA | 13691 | UGACAUGAAGUACCCCAUU |
| ETD02575 | 13371 | AGGAAAGUUUAUCCAGUAA | 13692 | UUACUGGAUAAACUUUCCU |
| ETD02576 | 13372 | UCUGCAAAAGUUGCACAAA | 13693 | UUUGUGCAACUUUUGCAGA |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02553 | 13373 | AAACUGAUAGCUGAGUCC | 13694 | GGACUCAGCUAUCAGUUU |
| ETD02554 | 13374 | UUGUGACAUGGAUGAAUC | 13695 | GAUUCAUCCAUGUCACAA |
| ETD02555 | 13375 | AGAAUACAGUGUUGGUCG | 13696 | CGACCAACACUGUAUUCU |
| ETD02556 | 13376 | AAUACAGUGUUGGUCGAU | 13697 | AUCGACCAACACUGUAUU |
| ETD02557 | 13377 | AUACAGUGUUGGUCGAUG | 13698 | CAUCGACCAACACUGUAU |
| ETD02558 | 13378 | UACAGUGUUGGUCGAUGU | 13699 | ACAUCGACCAACACUGUA |
| ETD02559 | 13379 | AUGUAAGCACACAAGUGA | 13700 | UCACUUGUGUGCUUACAU |
| ETD02560 | 13380 | UUUUUCAACCCCAGUAUC | 13701 | GAUACUGGGGUUGAAAAA |
| ETD02561 | 13381 | CUUUUUAUUCAAGAGCGA | 13702 | UCGCUCUUGAAUAAAAAG |
| ETD02562 | 13382 | UAUUCAAGAGCGAGAUGU | 13703 | ACAUCUCGCUCUUGAAUA |
| ETD02563 | 13383 | CAUUGCUUCAGGCAAUAA | 13704 | UUAUUGCCUGAAGCAAUG |
| ETD02564 | 13384 | AAUUACUUCGACAGCAGC | 13705 | GCUGCUGUCGAAGUAAUU |
| ETD02565 | 13385 | AUUACUUCGACAGCAGCA | 13706 | UGCUGCUGUCGAAGUAAU |
| ETD02566 | 13386 | CUUUUGUCAGUUGUGGUA | 13707 | UACCACAACUGACAAAAG |
| ETD02567 | 13387 | CUUGAUAAUACCUGUUGG | 13708 | CCAACAGGUAUUAUCAAG |
| ETD02568 | 13388 | UGAUAAUACCUGUUGGAA | 13709 | UUCCAACAGGUAUUAUCA |
| ETD02569 | 13389 | AAGAGGUGUUAUUAGAAU | 13710 | AUUCUAAUAACACCUCUU |
| ETD02570 | 13390 | UGUUACGAAAAAACUAUG | 13711 | CAUAGUUUUUUCGUAACA |
| ETD02571 | 13391 | AUCUGGCUGAGCAUAUUC | 13712 | GAAUAUGCUCAGCCAGAU |
| ETD02572 | 13392 | CUAUUCACUGCUAGCAAG | 13713 | CUUGCUAGCAGUGAAUAG |
| ETD02573 | 13393 | UAGUAAUGCAUGCCAUAC | 13714 | GUAUGGCAUGCAUUACUA |
| ETD02574 | 13394 | AAUGGGGUACUUCAUGUC | 13715 | GACAUGAAGUACCCCAUU |
| ETD02575 | 13395 | AGGAAAGUUUAUCCAGUA | 13716 | UACUGGAUAAACUUUCCU |
| ETD02576 | 13396 | UCUGCAAAAGUUGCACAA | 13717 | UUGUGCAACUUUUGCAGA |

TABLE 32
Relative GPAM mRNA Levels in Livers of Mice
Transfected with AAV8-TGB-h-GPAM - Part 1

| | | | Dose | Mean human GPAM mRNA level |
|---|---|---|---|---|
| Group | n | Treatment | (ug) | (Normalized to Group 1, Day 14) |
| 1 | 5 | PBS | | 1.00 |
| 2 | 5 | ETD02553 | 100 | 0.59 |
| 3 | 5 | ETD02554 | 100 | 0.96 |
| 4 | 5 | ETD02555 | 100 | 0.97 |
| 5 | 5 | ETD02556 | 100 | 0.99 |
| 6 | 5 | ETD02557 | 100 | 0.39 |
| 7 | 5 | ETD02558 | 100 | 0.75 |
| 8 | 5 | ETD02559 | 100 | 0.52 |
| 9 | 5 | ETD02560 | 100 | 0.70 |
| 10 | 5 | ETD02561 | 100 | 0.58 |
| 11 | 5 | ETD02562 | 100 | 0.28 |
| 12 | 5 | ETD02563 | 100 | 0.26 |
| 13 | 5 | ETD02564 | 100 | 0.36 |

TABLE 33
Relative GPAM mRNA Levels in Livers of Mice
Transfected with AAV8-TGB-h-GPAM - Part 2

| | | | Dose | Mean human GPAM mRNA level |
|---|---|---|---|---|
| Group | n | Treatment | (ug) | (Normalized to Group 1, Day 14) |
| 1 | 5 | PBS | | 1.00 |
| 2 | 5 | ETD02565 | 100 | 1.89 |
| 3 | 5 | ETD02566 | 100 | 1.49 |
| 4 | 5 | ETD02567 | 100 | 0.50 |
| 5 | 5 | ETD02568 | 100 | 0.40 |
| 6 | 5 | ETD02569 | 100 | 0.53 |
| 7 | 5 | ETD02570 | 100 | 0.77 |
| 8 | 5 | ETD02571 | 100 | 0.85 |
| 9 | 5 | ETD02572 | 100 | 0.21 |
| 10 | 5 | ETD02573 | 100 | 0.31 |
| 11 | 5 | ETD02574 | 100 | 0.26 |
| 12 | 5 | ETD02575 | 100 | 0.72 |
| 13 | 5 | ETD02576 | 100 | 0.33 |

### Example 18: Therapeutic siRNA-Mediated Knockdown of GPAM in a Mouse Model of Non-Alcoholic Fatty Liver Disease (NAFLD)

The effects of siRNA-mediated knockdown of GPAM in the liver was investigated in a high fat, high fructose diet-based mouse model of non-alcoholic fatty liver disease (NAFLD). Six- to eight-week-old C57BL/6NHsd female mice (Envigo) were given a Western high fat diet (Envigo TD. 120330, 0.2% cholesterol, 45% fat by calories) and high fructose water (55% fructose, 45% glucose) for eight weeks (Groups 2-4) or sixteen weeks (Groups 6-8) prior to siRNA injection.

After 8 weeks, mice in Group 1 on regular chow (n=5) and Group 2 on Western Diet (n=5) were injected with 100 ul of phosphate buffered saline (PBS) subcutaneously biweekly until 16 weeks (Day 112). Mice in Group 3 on Western Diet (n=5) were injected with 100 ug of siRNA ETD02282 targeting mouse GPAM and Group 4 on Western Diet (n=5) were injected with 100 ug of siRNA ETD02284 targeting mouse GPAM by subcutaneous injection biweekly until 16 weeks (Day 112) for a total of 4 injections. The siRNAs contain the GalNAc ligand ETL17 followed by a phosphorothioate linkage at the 5′ end of the sense strand. The siRNA sequences that were used are shown in Table 34, where Nf (e.g. Af, Cf, Gf, Tf, or Uf) is a 2′ fluoro-modified nucleoside, n (e.g. a, c, g, t, or u) is a 2′ O-methyl modified nucleoside, and “s” is a phosphorothioate linkage. The base sequences for each siRNA, with and without the 3′ UU extension, are shown in Table 35.

TABLE 34
Example siRNA Sequences

| | SEQ | Sense Strand Sequence (5′-3′) with | SEQ ID | |
|---|---|---|---|---|
| ETD# | ID NO: | GalNAc moiety | NO: | Antisense Strand Sequence (5′-3′) |
| ETD02282 | 12973 | [ETL17]scaauAfgAfcGfuuucuuaucasusu | 13080 | usGfsauAfaGfaaAfcGfuCfuAfuUfgsusu |
| ETD02284 | 12974 | [ETL17]scaauAfgAfcGfuuucuuaucasusu | 13081 | usGfsauAfaGfaAfacguCfuAfuUfgsusu |

TABLE 35
Example siRNA BASE Sequences

| siRNA | SEQ ID | Sense Strand Base Sequence | SEQ ID | Antisense Strand Base |
|---|---|---|---|---|
| Name | NO: | (5′ to 3′) | NO | Sequence (5′ to 3′) |
| ETD02282 | 13397 | CAAUAGACGUUUCUUAUCAUU | 13718 | UGAUAAGAAACGUCUAUUGUU |
| ETD02284 | 13398 | CAAUAGACGUUUCUUAUCAUU | 13719 | UGAUAAGAAACGUCUAUUGUU |
| | | Sense Strand Base | | Antisense Strand Base |
| siRNA | SEQ ID | Sequence (5′ to 3′), | SEQ ID | Sequence (5′ to 3′), |
| Name | NO: | without 3′ overhangs | NO | without 3′ overhangs |
| ETD02282 | 13399 | CAAUAGACGUUUCUUAUCA | 13720 | UGAUAAGAAACGUCUAUUG |
| ETD02284 | 13400 | CAAUAGACGUUUCUUAUCA | 13721 | UGAUAAGAAACGUCUAUUG |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02282 | 13401 | CAAUAGACGUUUCUUAUC | 13722 | GAUAAGAAACGUCUAUUG |
| ETD02284 | 13402 | CAAUAGACGUUUCUUAUC | 13723 | GAUAAGAAACGUCUAUUG |

After 16 weeks, mice in Group 5 on regular chow (n=5) and Group 6 on Western Diet (n=5) were injected with 100 ul of phosphate buffered saline (PBS) subcutaneously biweekly until 24 weeks (Day 168). Mice in Group 7 on Western Diet (n=5) were injected with 100 ug of siRNA ETD02282 targeting mouse GPAM and Group 8 on Western Diet (n=5) were injected with 100 ug of siRNA ETD02284 targeting mouse GPAM by subcutaneous injection biweekly until 24 weeks (Day 168) for a total of 4 injections. The

Body weights were recorded biweekly at the start of the Western diet until the end of the study. Mice were fasted 4-6 hours for serum collection prior to the first siRNA treatment and then 1 day prior to each biweekly siRNA injection. Serum was sent for the following clinical chemistry assays (IDEXX Laboratories, Incorporated): ALP, ALT, BUN, cholesterol, glucose, total bilirubin, total protein, triglycerides, and beta-hydroxybutarate. Ketones were measured in a drop of whole blood using Precision Xtra Ketone Test Strips (Abbott).

All mice in Groups 1-4 were euthanized via cervical dislocation following isoflurane exposure with a final serum bleed at 16 weeks and all mice in Groups 5-8 were euthanized via cervical dislocation following isoflurane exposure with a final serum bleed at 24 weeks and the liver was separated into 4 sections. One section was placed in RNAlater for qRT-PCR (Thermo Fisher #AM7020), one section was snap frozen in liquid nitrogen for liver triglyceride measurement, one section was placed into 10% formalin for fixation and paraffin embedding for H&E and Picrosirius Red staining, and one section was fixed with 10% formalin at 4C for 24 hours and then embedded in OCT for Oil Red O staining.

Total liver RNA was prepared by homogenizing the RNAlater liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The levels of liver GPAM mRNA were assessed by RT-qPCR in triplicate on a QuantStudio™ 6 Pro Real-Time PCR System using TaqMan assays for mouse GPAM (ThermoFisher, assay #Mm01261106_m1), mouse COL1A1 (ThermoFisher, assay #Mm00801666_g1), mouse TIMP1 (ThermoFisher, assay #Mm00801666_g1), mouse TGFB1 (ThermoFisher, assay #Mm01341361_m1), mouse Acta2/SMA (ThermoFisher, assay #Mm00725412_s1), mouse Tnf-α (ThermoFisher, assay #Mm00443258_m1), mouse II1B (ThermoFisher, assay #Mm00434228_m1), mouse CCL2 (ThermoFisher, assay #Mm00441242_m1), and the mouse housekeeping gene PPIA (ThermoFisher, assay #Mm02342430_g1) using PerfeCTa® qPCR FastMix®, Low ROX™ (VWR, Catalog #101419-222). Data was normalized to the mouse group on Western Diet receiving PBS (Group 2) by the delta-delta Ct method.

The 16 week GPAM mRNA levels are shown in Table. Data were normalized to the level in animals receiving PBS and on the Western Diet (Group 2). Mice on the Western Diet treated with ETD02282 (Group 3) or ETD02284 (Group 4) had reduced liver GPAM mRNA levels compared to mice on the Western Diet receiving PBS (Group 2). The mice on the Western Diet had elevated levels of GPAM compared to the mice on normal chow.

TABLE 36
16-week GPAM mRNA Levels in Mice Treated
with ETD02282 and ETD02284

| | | Relative | Mean Relative |
|---|---|---|---|
| | | Liver GPAM | Liver GPAM |
| Group # | Animal # | mRNA Level | mRNA level |
| 1 | 1 | 0.19 | 0.27 |
| | 2 | 0.34 | |
| | 3 | 0.23 | |
| | 4 | 0.21 | |
| | 5 | 0.48 | |
| 2 | 11 | 1.61 | 1.00 |
| | 12 | 0.65 | |
| | 13 | 1.07 | |
| | 14 | 0.47 | |
| | 15 | 1.89 | |
| 3 | 21 | 0.15 | 0.18 |
| | 22 | 0.16 | |
| | 23 | 0.19 | |
| | 24 | 0.30 | |
| | 25 | 0.13 | |
| 4 | 31 | 0.12 | 0.07 |
| | 32 | 0.13 | |
| | 33 | 0.02 | |
| | 34 | 0.19 | |
| | 35 | 0.02 | |

The 24-week GPAM mRNA levels are shown in Table 37. Data were normalized to the level in animals receiving PBS and on the Western Diet (Group 6). Mice on the Western Diet treated with ETD02282 (Group 7) or ETD02284 (Group 8) had reduced liver GPAM mRNA levels compared to mice on the Western Diet receiving PBS (Group 6). The mice on the Western Diet had elevated levels of GPAM compared to the mice on normal chow.

TABLE 37
24-week GPAM mRNA Levels in Mice Treated
with ETD02282 and ETD02284

| | | Relative | Mean Relative |
|---|---|---|---|
| | | Liver GPAM | Liver GPAM |
| Group # | Animal # | mRNA Level | mRNA level |
| 5 | 6 | 0.26 | 0.14 |
| | 7 | 0.20 | |
| | 8 | 0.04 | |
| | 9 | 0.21 | |
| | 10 | 0.14 | |
| 6 | 16 | 1.26 | 1.00 |
| | 17 | 1.30 | |
| | 18 | 0.67 | |
| | 19 | 1.27 | |
| | 20 | 0.96 | |
| 7 | 26 | 0.12 | 0.10 |
| | 27 | 0.20 | |
| | 28 | 0.18 | |
| | 29 | 0.06 | |
| | 30 | 0.04 | |
| 8 | 36 | 0.02 | 0.04 |
| | 37 | 0.25 | |
| | 38 | 0.02 | |
| | 39 | 0.01 | |
| | 40 | 0.16 | |

The starting (Day 0) and ending (Day 112) body weights are shown in Table 38 for the 16-week study. There was reduced weight gain in the mice fed the Western Diet and treated with ETD02284 (Group 4) compared to mice on the Western Diet and treated with PBS (Group 2) at 16 weeks.

TABLE 38
16-week Body Weights of Mice Treated
with ETD02282 and ETD02284

| | | Day 0 | | Day 112 | |
|---|---|---|---|---|---|
| | | Individual | Mean | Individual | Mean |
| | | Body | Body | Body | Body |
| | | Weight | Weight | Weight | Weight |
| Group # | Animal # | (g) | (g) | (g) | (g) |
| 1 | 1 | 17.7 | 17.48 | 21.8 | 23.14 |
| | 2 | 18.0 | | 26.23 | |
| | 3 | 17.5 | | 23.1 | |
| | 4 | 16.7 | | 22.13 | |
| | 5 | 17.5 | | 22.42 | |
| 2 | 11 | 17.3 | 18.12 | 31.28 | 32.08 |
| | 12 | 18.8 | | 35.24 | |
| | 13 | 18.1 | | 31.02 | |
| | 14 | 18.1 | | 32.04 | |
| | 15 | 18.3 | | 30.81 | |
| 3 | 21 | 18.0 | 18.74 | 33.08 | 34.01 |
| | 22 | 19.5 | | 34.41 | |
| | 23 | 18.3 | | 33.0 | |
| | 24 | 19.2 | | 34.46 | |
| | 25 | 18.7 | | 35.11 | |
| 4 | 31 | 18.3 | 18.32 | 28.30 | 29.07 |
| | 32 | 19.4 | | 34.27 | |
| | 33 | 17.9 | | 24.98 | |
| | 34 | 19.4 | | 30.52 | |
| | 35 | 16.6 | | 27.29 | |

The starting (Day 0) and ending (Day 168) body weights are shown in Table 39 for the 24-week study. There was no reduced weight gain in the mice fed the Western Diet and treated with ETD02282 (Group 7) or ETD02284 (Group 8) compared to mice on the Western Diet and treated with PBS (Group 6) at 24 weeks.

TABLE 39
24-week Body Weights of Mice Treated
with ETD02282 and ETD02284

| | | Day 0 | | Day 168 | |
|---|---|---|---|---|---|
| | | Individual | Mean | Individual | Mean |
| | | Body | Body | Body | Body |
| Group | Animal | Weight | Weight | Weight | Weight |
| # | # | (g) | (g) | (g) | (g) |
| 5 | 6 | 18.30 | 18.06 | 25.32 | 26.66 |
| | 7 | 19.00 | | 29.35 | |
| | 8 | 17.60 | | 25.35 | |
| | 9 | 18.80 | | 27.83 | |
| | 10 | 16.60 | | 25.45 | |
| 6 | 16 | 17.50 | 18.22 | 29.98 | 33.27 |
| | 17 | 18.10 | | 34.45 | |
| | 18 | 18.10 | | 33.14 | |
| | 19 | 18.70 | | 36.35 | |
| | 20 | 18.70 | | 35.44 | |
| 7 | 26 | 17.80 | 18.36 | 32.79 | 34.63 |
| | 27 | 19.10 | | 33.27 | |
| | 28 | 17.20 | | 34.99 | |
| | 29 | 18.00 | | 35.57 | |
| | 30 | 19.70 | | 36.42 | |
| 8 | 36 | 18.00 | 18.74 | 34.94 | 34.49 |
| | 37 | 19.40 | | 39.79 | |
| | 38 | 17.90 | | 34.46 | |
| | 39 | 19.60 | | 39.69 | |
| | 40 | 18.80 | | 23.56 | |

The 16-week serum levels of ALT, cholesterol, and ketones are shown in Table 40. The ALT and cholesterol levels at 16 weeks in mice treated with ETD02282 (Group 3) and ETD02284 (Group 4) were lower than mice treated with PBS (Group 2) on Western Diet. The ketone levels in mice at 16 weeks treated with ETD02284 (Group 4) were higher than mice treated with PBS (Group 2) on Western Diet.

TABLE 40
16-week ALT, Cholesterol, and Ketone Levels
in Mice Treated with ETD02282 and ETD02284

| | | | | Cholesterol | | | |
|---|---|---|---|---|---|---|---|
| | | ALT (U/L) | | (mg/dL) | | Ketones (mmol/L) | |
| Group | | Day 112 | | Day 112 | | Day 112 | |
| # | Animal # | Individual | Mean | Individual | Mean | Individual | Mean |
| 1 | 1 | 25 | 23.2 | 65 | 69.6 | | 0.55 |
| | 2 | 22 | | 80 | | 0.6 | |
| | 3 | 21 | | 74 | | 0.4 | |
| | 4 | 22 | | 64 | | 0.6 | |
| | 5 | 26 | | 65 | | 0.6 | |
| 2 | 11 | 118 | 82.4 | 208 | 187.2 | 0.4 | 0.3 |
| | 12 | 84 | | 178 | | 0.2 | |
| | 13 | 49 | | 163 | | 0.3 | |
| | 14 | 78 | | 199 | | 0.3 | |
| | 15 | 83 | | 188 | | 0.3 | |
| 3 | 21 | 34 | 24.4 | 137 | 143.4 | 0.3 | 0.36 |
| | 22 | 47 | | 146 | | 0.3 | |
| | 23 | 51 | | 129 | | 0.4 | |
| | 24 | 98 | | 154 | | 0.4 | |
| | 25 | 64 | | 148 | | 0.4 | |
| 4 | 31 | 22 | 20.2 | 132 | 140.6 | 0.6 | 0.56 |
| | 32 | 68 | | 151 | | 0.4 | |
| | 33 | 22 | | 133 | | 0.6 | |
| | 34 | 49 | | 146 | | 0.4 | |
| | 35 | 28 | | 141 | | 0.8 | |

The 24-week serum levels of ALT, cholesterol, and ketones are shown in Table 41. The ALT and cholesterol levels at 24 weeks in mice treated with ETD02282 (Group 7) and ETD02284 (Group 8) were lower than mice treated with PBS (Group 6) on Western Diet. The ketone levels in mice at 16 weeks treated with ETD02282 (Group 7) and ETD02284 (Group 8) were slightly higher than mice treated with PBS (Group 6) on Western Diet.

TABLE 41
24-week ALT, Cholesterol, and Ketone Levels
in Mice Treated with ETD02282 and ETD02284

| | | | | Cholesterol | | | |
|---|---|---|---|---|---|---|---|
| | | ALT (U/L) | | (mg/dL) | | Ketones (mmol/L) | |
| Group | | Day 168 | | Day 168 | | Day 168 | |
| # | Animal # | Individual | Mean | Individual | Mean | Individual | Mean |
| 5 | 6 | 22 | 22.2 | 77 | 72.4 | 0.4 | 0.54 |
| | 7 | 25 | | 79 | | 0.7 | |
| | 8 | 23 | | 57 | | 0.6 | |
| | 9 | 22 | | 63 | | 0.6 | |
| | 10 | 19 | | 86 | | 0.4 | |
| 6 | 16 | 54 | 70.8 | 131 | 136.4 | 0.4 | 0.48 |
| | 17 | 66 | | 134 | | 0.5 | |
| | 18 | 70 | | 116 | | 0.3 | |
| | 19 | 84 | | 160 | | 0.6 | |
| | 20 | 80 | | 141 | | 0.6 | |
| 7 | 26 | 70 | 40.8 | 118 | 105.8 | 0.6 | 0.52 |
| | 27 | 27 | | 96 | | 0.4 | |
| | 28 | 36 | | 105 | | 0.6 | |
| | 29 | 36 | | 120 | | 0.6 | |
| | 30 | 35 | | 90 | | 0.4 | |
| 8 | 36 | 57 | 44.0 | 124 | 107.8 | 0.7 | 0.56 |
| | 37 | 41 | | 106 | | 0.4 | |
| | 38 | 25 | | 138 | | 0.3 | |
| | 39 | 74 | | 122 | | 0.7 | |
| | 40 | 23 | | 49 | | 0.7 | |

To assess liver histology, liver sections were formalin fixed and paraffin embedded then stained with hematoxylin and eosin (H&E). The 0% steatotic area was calculated by measuring the white areas left behind by lipid droplets in the tissue divided by the total area of the tissue section. Five different liver serial sections were used per animal for quantification. The 16-week 0 steatotic area of the H&E stained formalin fixed liver sections is in Table 42. The 0 steatotic area at 16 weeks was reduced in mice treated with ETD02282 (Group 3) and ETD02284 (Group 4) compared to mice treated with PBS (Group 2) on Western Diet.

TABLE 42
16-week % Steatotic Area of Livers in
Mice Treated with ETD02282 and ETD02284

| | | Individual % | Mean % |
|---|---|---|---|
| Group # | Animal # | Steatotic Area | Steatotic Area |
| 2 | 11 | 9.05 | 7.91 |
| | 12 | 6.84 | |
| | 13 | 8.52 | |
| | 14 | 7.00 | |
| | 15 | 8.11 | |
| 3 | 21 | 4.76 | 6.10 |
| | 22 | 7.20 | |
| | 23 | 5.49 | |
| | 24 | 5.40 | |
| | 25 | 7.64 | |
| 4 | 31 | 3.54 | 4.93 |
| | 32 | 5.15 | |
| | 33 | 8.41 | |
| | 34 | 2.69 | |
| | 35 | 4.86 | |

To assess liver histology, liver sections were formalin fixed and paraffin embedded then stained with hematoxylin and eosin (H&E). The 0% steatotic area was calculated by measuring the white areas left behind by lipid droplets in the tissue divided by the total area of the tissue section. Five different liver serial sections were used per animal for quantification. The 24-week 00 steatotic area of the H&E stained formalin fixed liver sections is in Table 43. The % steatotic area at 24 weeks was reduced in mice treated with ETD02282 (Group 7) and ETD02284 (Group 8) compared to mice treated with PBS (Group 6) on Western Diet.

TABLE 43
24-week % Steatotic Area of Livers in
Mice Treated with ETD02282 and ETD02284

| | | Individual % | Mean % |
|---|---|---|---|
| Group # | Animal # | Steatotic Area | Steatotic Area |
| 6 | 16 | 10.48 | 12.10 |
| | 17 | 9.34 | |
| | 18 | 11.43 | |
| | 19 | 12.36 | |
| | 20 | 16.87 | |
| 7 | 26 | 8.34 | 8.23 |
| | 27 | 5.97 | |
| | 28 | 10.00 | |
| | 29 | 6.70 | |
| | 30 | 10.14 | |
| 8 | 36 | 6.61 | 5.95 |
| | 37 | 6.96 | |
| | 38 | 2.43 | |
| | 39 | 11.61 | |
| | 40 | 2.13 | |

Liver triglycerides were measured in apiece of flash frozen liver using a Triglyceride Colorimetric Kit (Elabscience PE-BC-K238) following the manufacturer's instructions. The relative levels of triglycerides in the flash frozen liver sections are shown in Table 44 with the data normalized to mice on normal diet treated with PBS (Group 1). The level of triglycerides in the liver at 16 weeks was reduced in mice treated with ETD02282 (Group 3) and ETD02284 (Group 4) compared to mice treated with PBS (Group 2) on Western Diet.

TABLE 44
16-week Liver Triglyceride Levels in Mice
Treated with ETD02282 and ETD02284

| | | Relative Liver | Relative Liver |
|---|---|---|---|
| | | Triglyceride | Triglyceride |
| Group # | Animal # | Level Individual | Level Mean |
| 1 | 1 | 0.92 | 1.00 |
| | 2 | 0.53 | |
| | 3 | 1.35 | |
| | 4 | 1.33 | |
| | 5 | 0.87 | |
| 2 | 11 | 0.98 | 1.62 |
| | 12 | 2.32 | |
| | 13 | 1.17 | |
| | 14 | 1.57 | |
| | 15 | 2.06 | |
| 3 | 21 | 1.79 | 1.11 |
| | 22 | 1.57 | |
| | 23 | 0.87 | |
| | 24 | 0.76 | |
| | 25 | 1.07 | |
| 4 | 31 | 0.61 | 0.79 |
| | 32 | 0.27 | |
| | 33 | 0.56 | |
| | 34 | 1.55 | |
| | 35 | 0.94 | |

Liver triglycerides were measured in a piece of flash frozen liver using a Triglyceride Colorimetric Kit (Elabscience #E-BC-K238) following the manufacturer's instructions. The levels of triglycerides in the flash frozen liver sections are shown in Table 45 with the data normalized to mice on normal diet treated with PBS (Group 5). The level of triglycerides in the liver at 24 weeks was not reduced in mice treated with ETD02282 (Group 7) and ETD02284 (Group 8) compared to mice treated with PBS (Group 6) on Western Diet.

TABLE 45
24-week Liver Triglyceride Levels in Mice
Treated with ETD02282 and ETD02284

| | | Relative Liver | Relative Liver |
|---|---|---|---|
| | | Triglyceride | Triglyceride |
| Group # | Animal # | Level Individual | Level Mean |
| 5 | 6 | 0.78 | 1.00 |
| | 7 | 1.14 | |
| | 8 | 0.93 | |
| | 9 | 0.97 | |
| | 10 | 1.18 | |
| 6 | 16 | 1.23 | 1.36 |
| | 17 | 1.01 | |
| | 18 | 1.41 | |
| | 19 | 1.87 | |
| | 20 | 1.27 | |
| 7 | 26 | 2.48 | 1.80 |
| | 27 | 2.48 | |
| | 28 | 1.74 | |
| | 29 | 0.77 | |
| | 30 | 2.87 | |
| 8 | 36 | 0.43 | 1.01 |
| | 37 | 1.34 | |
| | 38 | 0.96 | |
| | 39 | 1.29 | |
| | 40 | 1.01 | |

Expression of fibrotic and pro-inflammatory genes in the liver were measured by qRT-PCR and normalized to the mice on Western Diet receiving PBS (Group 2). The expression of pro-fibrotic genes COL1A1 and TIMP1 and pro-inflammatory genes TNF-α, IL1b, and CCL2 are shown in Table 46. Mice on Western Diet treated with ETD02284 (Group 4) had reduced expression of pro-fibrotic genes COL1A1 and TIMP1 and pro-inflammatory genes TNF-alpha and CCL2 at 16 weeks compared to mice treated with PBS (Group 2) on Western Diet. Mice treated with ETD02282 (Group 3) did not show reduction of any genes compared to PBS treated mice (Group 2) except TIMP1.

TABLE 46
16-week Expression of Pro-fibrotic and Pro-inflammatory
Genes in Mice Treated with ETD02282 and ETD02284

| | | COL1A1 | | TIMP1 | | TNF- alpha | | IL1b | | CCL2 | |
|---|---|---|---|---|---|---|---|---|---|---|---|
| Group | | Day 112 | | Day 112 | | Day 112 | | Day 112 | | Day 112 | |
| # | Animal # | Individual | Mean | Individual | Mean | Individual | Mean | Individual | Mean | Individual | Mean |
| 1 | 1 | 0.12 | 0.19 | 0.004 | 0.004 | 0.20 | 0.22 | 0.63 | 1.11 | 0.11 | 0.33 |
| | 2 | 0.30 | | 0.002 | | 0.25 | | 0.93 | | 0.38 | |
| | 3 | 0.11 | | 0.03 | | 1.72 | | 3.82 | | 0.36 | |
| | 4 | 0.14 | | 0.002 | | 0.10 | | 0.89 | | 0.39 | |
| | 5 | 0.49 | | 0.002 | | 0.05 | | 0.85 | | 0.67 | |
| 2 | 11 | 1.24 | 1.00 | 1.21 | 1.00 | 0.09 | 1.00 | 0.27 | 1.00 | 0.36 | 1.00 |
| | 12 | 0.92 | | 0.58 | | 0.35 | | 1.52 | | 1.20 | |
| | 13 | 0.73 | | — | | 30.96 | | — | | — | |
| | 14 | 0.75 | | 0.96 | | 0.41 | | 1.67 | | 1.76 | |
| | 15 | 1.60 | | 1.48 | | 2.48 | | 1.45 | | 1.32 | |
| 3 | 21 | 0.73 | 1.51 | 0.26 | 0.72 | 0.18 | 1.74 | 0.47 | 4.86 | 0.69 | 2.52 |
| | 22 | 2.19 | | 0.88 | | 2.86 | | 8.42 | | 2.84 | |
| | 23 | 1.40 | | — | | — | | 13.30 | | 4.06 | |
| | 24 | 2.16 | | 1.04 | | 3.27 | | 10.63 | | 3.26 | |
| | 25 | 1.65 | | 1.14 | | 5.48 | | 4.88 | | 3.92 | |
| 4 | 31 | 0.37 | 0.29 | 1.16 | 0.15 | 1.20 | 0.30 | 1.14 | 1.10 | 0.58 | 0.51 |
| | 32 | 1.30 | | 0.61 | | 1.26 | | 2.02 | | 1.56 | |
| | 33 | 0.08 | | 0.01 | | 0.09 | | 0.64 | | 0.40 | |
| | 34 | 0.90 | | 0.37 | | 0.14 | | 0.83 | | 0.27 | |
| | 35 | 0.07 | | 0.03 | | 0.14 | | 1.32 | | 0.36 | |

Expression of fibrotic and pro-inflammatory genes in the liver were measured by qRT-PCR and normalized to the mice on Western Diet receiving PBS (Group 2). The expression of pro-fibrotic genes COL1A1 and TIMP1 and pro-inflammatory genes TNF-α, IL1b, and CCL2 are shown in Table 47. Mice on Western Diet treated with ETD02282 (Group 7) and ETD02284 (Group 8) had reduced expression of pro-fibrotic genes COL1A11 and TIMP1 and pro-inflammatory gene TNF-alpha at 24 weeks compared to mice treated with PBS (Group 6). Mice treated with either siRNA did not show any significant change in expression of pro-inflammatory markers IL1b or CCL2 at 24 weeks.

TABLE 47
24-week Expression of Pro-fibrotic and Pro-inflammatory
Genes in Mice Treated with ETD02282 and ETD02284

| | | COL1A1 | | TIMP1 | | TNF-alpha | | IL1b | | CCL2 | |
|---|---|---|---|---|---|---|---|---|---|---|---|
| Group | | Day 168 | | Day 168 | | Day 168 | | Day 168 | | Day 168 | |
| # | Animal # | Individual | Mean | Individual | Mean | Individual | Mean | Individual | Mean | Individual | Mean |
| 5 | 6 | 0.51 | 0.07 | 0.004 | 0.003 | 0.38 | 0.10 | 0.86 | 0.50 | 0.34 | 0.32 |
| | 7 | 0.08 | | 0.001 | | 0.17 | | 0.29 | | 0.27 | |
| | 8 | 0.002 | | 0 | | 0.04 | | 0.55 | | 0.44 | |
| | 9 | 0.32 | | 0.003 | | 0.24 | | 0.87 | | 0.34 | |
| | 10 | 0.08 | | 0 | | 0.01 | | 0.26 | | 0.26 | |
| 6 | 16 | 1.52 | 1.00 | 0.87 | 1.00 | 2.13 | 1.00 | 0.77 | 1.00 | 0.81 | 1.00 |
| | 17 | 1.84 | | 1.12 | | 1.32 | | 0.45 | | 1.08 | |
| | 18 | 0.26 | | 0.85 | | 0.71 | | 1.68 | | 0.99 | |
| | 19 | 2.19 | | 1.38 | | 0.98 | | 0.99 | | 1.37 | |
| | 20 | 0.63 | | 0.88 | | 0.51 | | 1.76 | | 0.84 | |
| 7 | 26 | 0.43 | 0.15 | 0.29 | 0.18 | 0.77 | 0.20 | 0.30 | 0.95 | 0.80 | 0.71 |
| | 27 | 0.16 | | 0.18 | | 0.32 | | 1.07 | | 0.88 | |
| | 28 | 0.17 | | 0.46 | | 0.26 | | 2.44 | | 0.59 | |
| | 29 | 0.10 | | 0.20 | | 0.18 | | 1.27 | | 1.07 | |
| | 30 | 0.07 | | 0.04 | | 0.03 | | 0.76 | | 0.41 | |
| 8 | 36 | 0.06 | 0.43 | 0.86 | 0.56 | 0.44 | 0.47 | 0.64 | 1.10 | 0.96 | 1.06 |
| | 37 | 2.15 | | 0.63 | | 0.43 | | 0.22 | | 0.56 | |
| | 38 | 0.53 | | 0.36 | | 0.19 | | 1.06 | | 2.69 | |
| | 39 | 0.12 | | 1.65 | | 0.41 | | 1.14 | | 1.08 | |
| | 40 | 1.72 | | 0.17 | | 1.60 | | 9.19 | | 0.87 | |

### Example 19. Preventative siRNA-Mediated Knockdown of GPAM in a Mouse Model of Non-Alcoholic Fatty Liver Disease (NAFLD)

The protective effects of siRNA-mediated knockdown of GPAM in the liver was investigated in a high fat, high fructose diet-based mouse model of non-alcoholic fatty liver disease (NAFLD). Six- to eight-week-old C57BL/6NHsd female mice (Envigo) were given a Western high fat diet (Envigo TD. 120330, 0.2% cholesterol, 45% fat by calories) and high fructose water (55% fructose, 45% glucose). Mice were on diet for eight weeks (Groups 2-6) or sixteen weeks (Groups 8-12) with the following siRNAs targeting GPAM starting Day 0 of the study: Group 1 (n=5) on regular chow were injected with 100 ul of phosphate buffered saline (PBS) subcutaneously biweekly until 8 weeks (Day 56) or Group 7 (n=5) until 16 weeks (Day 112). Group 2 (n=5) on the Western diet were injected with 100 ul of phosphate buffered saline (PBS) subcutaneously biweekly until 8 weeks (Day 56) or Group 8 (n=5) until 16 weeks (Day 112).

Group 3 (n=5) on the Western diet were injected with 200 ug of siRNA ETD02282 targeting mouse GPAM subcutaneously biweekly until 8 weeks (Day 56) or Group 9 (n=5) until 16 weeks (Day 112). Group 4 (n=5) on the Western diet were injected with 60 ug of siRNA ETD02282 targeting mouse GPAM subcutaneously biweekly until 8 weeks (Day 56) or Group 10 (n=5) until 16 weeks (Day 112). Group 5 (n=5) on the Western diet were injected with 200 ug of siRNA ETD02284 targeting mouse GPAM subcutaneously biweekly until 8 weeks (Day 56) or Group 11 (n=5) until 16 weeks (Day 112). Group 6 (n=5) on the Western diet were injected with 60 ug of siRNA ETD02284 targeting mouse GPAM subcutaneously biweekly until 8 weeks (Day 56) or Group 12 (n=5) until 16 weeks (Day 112). Mice on the 8 week study received a total of 4 siRNA doses and mice on the 16 week study received a total of 16 siRNA doses. The siRNA sequences that were used are shown in Table 48, where Nf (e.g. Af, Cf, Gf, Tf, or Uf) is a 2′ fluoro-modified nucleoside, n (e.g. a, c, g, t, or u) is a 2′ O-methyl modified nucleoside, and “s” is a phosphorothioate linkage. The base sequences for each siRNA, with and without the 3′ UU extension, are shown in Table 49

TABLE 48
Example siRNA Sequences

| | SEQ | Sense Strand Sequence (5′-3′) with | SEQ ID | |
|---|---|---|---|---|
| ETD# | ID NO: | GalNAc moiety | NO: | Antisense Strand Sequence (5′-3′) |
| ETD02282 | 12973 | [ETL17]scaauAfgAfcGfuuucuuaucasusu | 13080 | usGfsauAfaGfaaAfcGfuCfuAfuUfgsusu |
| ETD02284 | 12974 | [ETL17]scaauAfgAfcGfuuucuuaucasusu | 13081 | usGfsauAfaGfaAfacguCfuAfuUfgsusu |

TABLE 49
Example siRNA BASE Sequences

| siRNA | SEQ ID | Sense Strand Base | SEQ ID | Antisense Strand Base |
|---|---|---|---|---|
| Name | NO: | Sequence (5′ to 3′) | NO | Sequence (5′ to 3′) |
| ETD02282 | 13397 | CAAUAGACGUUUCUUAUCAUU | 13718 | UGAUAAGAAACGUCUAUUGUU |
| ETD02284 | 13398 | CAAUAGACGUUUCUUAUCAUU | 13719 | UGAUAAGAAACGUCUAUUGUU |
| | | Sense Strand Base | | Antisense Strand Base |
| siRNA | SEQ ID | Sequence (5′ to 3′), | SEQ ID | Sequence (5′ to 3′), |
| Name | NO: | without 3′ overhangs | NO | without 3′ overhangs |
| ETD02282 | 13399 | CAAUAGACGUUUCUUAUCA | 13720 | UGAUAAGAAACGUCUAUUG |
| ETD02284 | 13400 | CAAUAGACGUUUCUUAUCA | 13721 | UGAUAAGAAACGUCUAUUG |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02282 | 13401 | CAAUAGACGUUUCUUAUC | 13722 | GAUAAGAAACGUCUAUUG |
| ETD02284 | 13402 | CAAUAGACGUUUCUUAUC | 13723 | GAUAAGAAACGUCUAUUG |

Body weights were recorded biweekly until the end of the study. Mice were fasted 4-6 hours for serum collection on Day 0 and then 1 day prior to each biweekly siRNA injection. Serum was sent for the following clinical chemistry assays performed at IDEXX Laboratories, Incorporated—ALP, ALT, BUN, cholesterol, glucose, total bilirubin, total protein, triglycerides, and beta-hydroxybutarate.

All mice in Groups 1-6 were euthanized via cervical dislocation following isoflurane exposure with a final serum bleed at 8 weeks and all mice in Groups 7-12 were euthanized via cervical dislocation following isoflurane exposure with a final serum bleed at 16 weeks and the liver was separated into 4 sections. One section was placed in RNAlater for qRT-PCR (Thermo Fisher #AM7020), one section was snap frozen in liquid nitrogen for liver triglyceride measurement, one section was placed into 10% formalin for fixation and paraffin embedding for H&E and Picrosirius Red staining, and one section was fixed with 10% formalin at 4C for 24 hours and then embedded in OCT for Oil Red 0 staining.

Total liver RNA was prepared by homogenizing the RNAlater liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The levels of liver GPAM mRNA were assessed by RT-qPCR in triplicate on a QuantStudio™ 6 Pro Real-Time PCR System using TaqMan assays for mouse GPAM (ThermoFisher, assay #Mm01261106_m1), mouse COL1A1 (ThermoFisher, assay #Mm00801666_g1), mouse TIMP1 (ThermoFisher, assay #Mm00801666_g1), mouse TGFB1 (ThermoFisher, assay #Mm01341361_m1), mouse Acta2/SMA (ThermoFisher, assay #Mm00725412_s1), mouse Tnf-α (ThermoFisher, assay #Mm00443258_m1), mouse II1B (ThermoFisher, assay #Mm00434228_m1), mouse CCL2 (ThermoFisher, assay #Mm00441242_m1), and the mouse housekeeping gene PPIA (ThermoFisher, assay #Mm02342430_g1) using PerfeCTa® qPCR FastMix®, Low ROX™ (VWR, Catalog #101419-222). Data was normalized to the mouse group on Western Diet receiving PBS (Group 2) using the delta-delta Ct method.

The 8-week GPAM mRNA levels are shown in Table. Data were normalized to the level in animals receiving PBS and on the Western Diet (Group 2). Mice on the Western Diet treated with ETD02282 (Groups 3 and 4) or ETD02284 (Groups 5 and 6) had reduced liver GPAM mRNA levels compared to mice on the Western Diet receiving PBS (Group 2). The mice on the Western Diet had elevated levels of GPAM compared to the mice on normal chow.

TABLE 50
8-week GPAM mRNA Levels in Mice Treated
with ETD02282 and ETD02284

| | | Relative | Mean Relative |
|---|---|---|---|
| | | Liver GPAM | Liver GPAM |
| Group # | Animal # | mRNA Level | mRNA level |
| 1 | 1 | 0.83 | 0.61 |
| | 2 | 0.54 | |
| | 3 | 0.81 | |
| | 4 | 0.49 | |
| | 5 | 0.47 | |
| 2 | 11 | 2.17 | 1.00 |
| | 12 | 1.38 | |
| | 13 | 0.60 | |
| | 14 | 0.82 | |
| | 15 | 0.65 | |
| 3 | 21 | 0.09 | 0.13 |
| | 22 | 0.13 | |
| | 23 | 0.06 | |
| | 24 | 0.15 | |
| | 25 | 0.33 | |
| 4 | 31 | 0.08 | 0.09 |
| | 32 | 0.04 | |
| | 33 | 0.21 | |
| | 34 | 0.09 | |
| | 35 | 0.10 | |
| 5 | 41 | 0.06 | 0.05 |
| | 42 | 0.01 | |
| | 43 | 0.13 | |
| | 44 | 0.05 | |
| | 45 | 0.04 | |
| 6 | 51 | 0.05 | 0.11 |
| | 52 | 0.17 | |
| | 53 | 0.17 | |
| | 54 | 0.12 | |
| | 55 | 0.09 | |

The 16-week GPAM mRNA levels are shown in Table 51. Data were normalized to the level in animals receiving PBS and on the Western Diet (Group 8). Mice on the Western Diet treated with ETD02292 (Groups 9 and 10) or ETD02284 (Groups 11 and 12) had reduced liver GPAM mRNA levels compared to mice on the Western Diet receiving PBS (Group 8). The mice on the Western Diet had elevated levels of GPAM compared to the mice on normal chow.

TABLE 51
16-week GPAM mRNA Levels in Mice Treated
with ETD02282 and ETD02284

| | | Relative | Mean Relative |
|---|---|---|---|
| | | Liver GPAM | Liver GPAM |
| Group # | Animal # | mRNA Level | mRNA level |
| 7 | 6 | 0.96 | 0.62 |
| | 7 | 0.57 | |
| | 8 | 0.87 | |
| | 9 | 0.35 | |
| | 10 | 0.57 | |
| 8 | 16 | 0.61 | 1.00 |
| | 17 | 1.26 | |
| | 18 | 0.93 | |
| | 19 | 1.28 | |
| | 20 | 1.10 | |
| 9 | 26 | 0.08 | 0.17 |
| | 27 | 0.35 | |
| | 28 | 0.12 | |
| | 29 | 0.52 | |
| | 30 | 0.09 | |
| 10 | 36 | 0.24 | 0.17 |
| | 37 | 0.18 | |
| | 38 | 0.25 | |
| | 39 | 0.22 | |
| | 40 | 0.05 | |
| 11 | 46 | 0.04 | 0.09 |
| | 47 | 0.08 | |
| | 48 | 0.19 | |
| | 49 | 0.22 | |
| | 50 | 0.03 | |
| 12 | 56 | 0.13 | 0.14 |
| | 57 | 0.47 | |
| | 58 | 0.11 | |
| | 59 | 0.09 | |
| | 60 | 0.08 | |

The starting (Day 0) and ending (Day 56) body weights are shown in Table 52 for the 8-week study. There was reduced weight gain in the mice fed the Western Diet and treated with both doses of ETD02284 (Groups 5 and 6) compared to mice on the Western Diet and treated with PBS (Group 2) at 8 weeks.

TABLE 52
8-week Body Weights of Mice Treated
with ETD02282 and ETD02284

| | | Day 0 | | Day 56 | |
|---|---|---|---|---|---|
| | | Individual | Mean | Individual | Mean |
| | | Body | Body | Body | Body |
| | | Weight | Weight | Weight | Weight |
| Group # | Animal # | (g) | (g) | (g) | (g) |
| 1 | 1 | 20.8 | 20.44 | 23.6 | 23.30 |
| | 2 | 19.7 | | 22.9 | |
| | 3 | 19.0 | | 21.3 | |
| | 4 | 20.7 | | 24.5 | |
| | 5 | 22.0 | | 24.3 | |
| 2 | 11 | 18.5 | 18.56 | 27.0 | 27.68 |
| | 12 | 17.9 | | 27.8 | |
| | 13 | 18.7 | | 28.0 | |
| | 14 | 18.6 | | 29.8 | |
| | 15 | 19.1 | | 25.8 | |
| 3 | 21 | 19.7 | 19.28 | 33.6 | 26.20 |
| | 22 | 19.2 | | 24.8 | |
| | 23 | 19.6 | | 22.8 | |
| | 24 | 19.3 | | 27.5 | |
| | 25 | 18.6 | | 22.3 | |
| 4 | 31 | 19.8 | 20.02 | 30.5 | 28.44 |
| | 32 | 21.0 | | 27.7 | |
| | 33 | 19.0 | | 27.4 | |
| | 34 | 21.1 | | 30.5 | |
| | 35 | 19.2 | | 26.1 | |
| 5 | 41 | 18.5 | 18.42 | 21.3 | 25.50 |
| | 42 | 17.0 | | 20.9 | |
| | 43 | 19.0 | | 27.6 | |
| | 44 | 19.0 | | 26.4 | |
| | 45 | 18.6 | | 31.3 | |
| 6 | 51 | 17.3 | 18.7 | 22.7 | 25.84 |
| | 52 | 18.8 | | 29.4 | |
| | 53 | 20.1 | | 23.1 | |
| | 54 | 19.2 | | 23.4 | |
| | 55 | 18.1 | | 30.6 | |

The starting (Day 0) and ending (Day 112) body weights are shown in Table 53 for the 16-week study. There was no reduced weight gain in the mice fed the Western Diet and treated with the 200 ug dose of ETD02282 (Group 9) or ETD02284 (Group 11) compared to mice on the Western Diet and treated with PBS (Group 8) at 16 weeks. However, there was reduced weight gain in mice fed the Western Diet and treated with 60 ug dose of ETD02282 (Group 10) and ETD02284 (Group 12).

TABLE 53
16-week Body Weights of Mice Treated
with ETD02282 and ETD02284

| | | Day 0 | | Day 112 | |
|---|---|---|---|---|---|
| | | Individual | Mean | Individual | Mean |
| | | Body | Body | Body | Body |
| | | Weight | Weight | Weight | Weight |
| Group # | Animal # | (g) | (g) | (g) | (g) |
| 7 | 6 | 22.93 | 21.44 | 26.78 | 27.54 |
| | 7 | 21.49 | | 30.33 | |
| | 8 | 19.88 | | 27.17 | |
| | 9 | 22.13 | | 27.05 | |
| | 10 | 20.76 | | 26.35 | |
| 8 | 16 | 19.25 | 20.06 | 30.36 | 33.53 |
| | 17 | 21.26 | | 39.01 | |
| | 18 | 17.38 | | 27.02 | |
| | 19 | 20.90 | | 33.18 | |
| | 20 | 21.52 | | 38.09 | |
| 9 | 26 | 21.46 | 20.54 | 38.82 | 34.61 |
| | 27 | 19.21 | | 29.35 | |
| | 28 | 20.53 | | 30.10 | |
| | 29 | 19.61 | | 34.82 | |
| | 30 | 21.88 | | 39.95 | |
| 10 | 36 | 20.54 | 19.23 | 34.60 | 30.07 |
| | 37 | 20.45 | | 34.13 | |
| | 38 | 17.69 | | 25.50 | |
| | 39 | 18.21 | | 22.77 | |
| | 40 | 19.27 | | 33.37 | |
| 11 | 46 | 21.44 | 21.28 | 32.61 | 37.04 |
| | 47 | 22.3 | | 44.75 | |
| | 48 | 19.55 | | 32.44 | |
| | 49 | 22.53 | | 39.20 | |
| | 50 | 20.60 | | 36.21 | |
| 12 | 56 | 19.72 | 19.00 | 26.28 | 27.39 |
| | 57 | 18.74 | | 30.20 | |
| | 58 | 19.44 | | 30.17 | |
| | 59 | 19.00 | | 26.95 | |
| | 60 | 18.09 | | 23.35 | |

The 8-week serum levels of ALT, cholesterol, and ketones are shown in Table 54. The ALT levels at 8 weeks in mice treated with both doses of ETD02282 (Groups 3 and 4) and ETD02284 (Groups 5 and 6) were lower than mice treated with PBS (Group 2) on Western Diet. The serum cholesterol levels at 8 weeks in mice treated with either ETD02282 (Groups 3 and 4) or ETD02284 (Groups 5 and 6) on Western Diet were not significantly decreased compared to mice treated with PBS (Group 2) on Western Diet. The ketone levels in mice at 8 weeks treated with both doses of ETD02284 (Groups 5 and 6) on Western Diet were higher than mice treated with PBS (Group 2) on Western Diet while the mice treated with both doses of ETD02282 (Groups 3 and 4) did not show an increase in ketones.

TABLE 54
8-week ALT, Cholesterol, and Ketone Levels
in Mice Treated with ETD02282 and ETD02284

| | | | | Cholesterol | | | |
|---|---|---|---|---|---|---|---|
| | | ALT (U/L) | | (mg/dL) | | Ketones (mmol/L) | |
| Group | | Day 56 | | Day 56 | | Day 56 | |
| # | Animal # | Individual | Mean | Individual | Mean | Individual | Mean |
| 1 | 1 | 18 | 18.4 | 72 | 65.4 | 0.3 | 0.4 |
| | 2 | 18 | | 70 | | 0.4 | |
| | 3 | 19 | | 60 | | 0.4 | |
| | 4 | 18 | | 63 | | 0.4 | |
| | 5 | 19 | | 62 | | 0.5 | |
| 2 | 11 | 24 | 32.8 | 100 | 117.0 | 0.4 | 0.42 |
| | 12 | 31 | | 105 | | 0.4 | |
| | 13 | 40 | | 150 | | 0.4 | |
| | 14 | 36 | | 113 | | 0.3 | |
| | 15 | 33 | | 117 | | 0.6 | |
| 3 | 21 | 42 | 25.4 | 134 | 119.8 | 0.6 | 0.5 |
| | 22 | 22 | | 131 | | 0.4 | |
| | 23 | 21 | | 116 | | 0.7 | |
| | 24 | 19 | | 104 | | 0.3 | |
| | 25 | 23 | | 114 | | 0.5 | |
| 4 | 31 | 28 | 25.8 | 136 | 115.6 | 0.4 | 0.4 |
| | 32 | 25 | | 116 | | 0.5 | |
| | 33 | 25 | | 101 | | 0.4 | |
| | 34 | 28 | | 112 | | 0.3 | |
| | 35 | 23 | | 113 | | 0.4 | |
| 5 | 41 | 14 | 29.2 | 124 | 147.0 | 1.0 | 0.72 |
| | 42 | 25 | | 124 | | 0.8 | |
| | 43 | 28 | | 174 | | 0.6 | |
| | 44 | 43 | | 152 | | 0.6 | |
| | 45 | 36 | | 161 | | 0.6 | |
| 6 | 51 | 19 | 24.0 | 118 | 112.2 | 0.8 | 0.58 |
| | 52 | 27 | | 129 | | 0.5 | |
| | 53 | 23 | | 78 | | 0.6 | |
| | 54 | 17 | | 114 | | 0.6 | |
| | 55 | 34 | | 122 | | 0.4 | |

The 16-week serum levels of ALT, cholesterol, and ketone betahydroxybutyrate (BHB) are shown in Table 55. The ALT levels at 16 weeks in mice treated with both doses of ETD02282 (Groups 9 and 10) and ETD02284 (Groups 11 and 12) were lower than mice treated with PBS (Group 8) on Western Diet. The serum cholesterol levels at 16 weeks in mice treated with either ETD02282 (Groups 9 and 10) or ETD02284 (Groups 11 and 12) on Western Diet increased compared to mice treated with PBS (Group 8) on Western Diet. The (BHB) serum levels at 16 weeks were not increased with either siRNA treatment or dosage.

TABLE 55
16-week ALT, Cholesterol, and Ketone Levels
in Mice Treated with ETD02282 and ETD02284

| | | | | Cholesterol | | | |
|---|---|---|---|---|---|---|---|
| | | ALT (U/L) | | (mg/dL) | | BHB (mg/dL) | |
| Group | | Day 112 | | Day 112 | | Day 112 | |
| # | Animal # | Individual | Mean | Individual | Mean | Individual | Mean |
| 7 | 6 | 25 | 23.6 | 66 | 66.2 | 4.7 | 4.08 |
| | 7 | 23 | | 66 | | 2.7 | |
| | 8 | 25 | | 66 | | 4.1 | |
| | 9 | 21 | | 61 | | 4.7 | |
| | 10 | 24 | | 72 | | 4.2 | |
| 8 | 16 | 70 | 96.8 | 138 | 111.8 | 8.9 | 6.78 |
| | 17 | 172 | | 72 | | 8.3 | |
| | 18 | 41 | | 125 | | 3.9 | |
| | 19 | 125 | | 154 | | 8.2 | |
| | 20 | 76 | | 70 | | 4.6 | |
| 9 | 26 | 62 | 46.0 | 133 | 111.8 | 5.3 | 5.62 |
| | 27 | 40 | | 129 | | 4.0 | |
| | 28 | 36 | | 129 | | 5.0 | |
| | 29 | 37 | | 105 | | 7.2 | |
| | 30 | 55 | | 63 | | 6.6 | |
| 10 | 36 | 50 | 53.6 | 135 | 139.2 | 4.9 | 3.96 |
| | 37 | 55 | | 155 | | 4.8 | |
| | 38 | 41 | | 112 | | 2.9 | |
| | 39 | 23 | | 125 | | 4.4 | |
| | 40 | 99 | | 169 | | 2.8 | |
| 11 | 46 | 49 | 77.8 | 174 | 173.4 | 5.5 | 5.02 |
| | 47 | 97 | | 207 | | 8.5 | |
| | 48 | 52 | | 156 | | 5.8 | |
| | 49 | 123 | | 158 | | 3.0 | |
| | 50 | 68 | | 172 | | 2.3 | |
| 12 | 56 | 24 | 31.4 | 129 | 123.8 | 14.8 | 6.96 |
| | 57 | 43 | | 136 | | 5.2 | |
| | 58 | 41 | | 126 | | 5.8 | |
| | 59 | 28 | | 118 | | 4.5 | |
| | 60 | 21 | | 110 | | 4.5 | |

To assess liver histology, liver sections were formalin fixed and paraffin embedded then stained with hematoxylin and eosin (H&E). The % steatotic area was calculated by measuring the white areas left behind by lipid droplets in the tissue divided by the total area of the tissue section. Five different liver serial sections were used per animal for quantification. The 8-week % steatotic area of the H&E stained formalin fixed liver sections is in Table 56. Mice on the Western diet treated with the 200 ug dose of ETD02282 (Group 3) and both doses of ETD02284 (Groups 5 and 6) had reduced % steatotic area compared to mice received PBS (Group 2) on Western Diet. The mice treated with ETD02284 had a greater reduction in steatotic area than the mice treated with ETD02282,

TABLE 56
8-week % Steatotic Area of Livers in Mice
Treated with ETD02282 and ETD02284

| | | Individual % | Mean % |
|---|---|---|---|
| Group # | Animal # | Steatotic Area | Steatotic Area |
| 2 | 11 | 1.81 | 2.59 |
| | 12 | 2.74 | |
| | 13 | 2.52 | |
| | 14 | 4.20 | |
| | 15 | 1.67 | |
| 3 | 21 | 2.45 | 1.69 |
| | 22 | 1.27 | |
| | 23 | 2.05 | |
| | 24 | 1.68 | |
| | 25 | 1.00 | |
| 4 | 31 | 2.58 | 2.81 |
| | 32 | 2.45 | |
| | 33 | 2.71 | |
| | 34 | 4.49 | |
| | 35 | 1.83 | |
| 5 | 41 | 0.80 | 1.55 |
| | 42 | 0.76 | |
| | 43 | 1.52 | |
| | 44 | 2.71 | |
| | 45 | 1.96 | |
| 6 | 51 | 1.01 | 1.52 |
| | 52 | 1.23 | |
| | 53 | 1.95 | |
| | 54 | 2.83 | |
| | 55 | 0.56 | |

To assess liver histology, liver sections were formalin fixed and paraffin embedded then stained with hematoxylin and eosin (H&E). The % steatotic area was calculated by measuring the white areas left behind by lipid droplets in the tissue divided by the total area of the tissue section. Five different liver serial sections were used per animal for quantification. The 16-week 00 steatotic area of the H&E stained formalin fixed liver sections is in Table 57. Mice on the Western Diet treated with both doses of ETD02282 (Groups 9 and 10) and ETD02284 (Groups 11 and 12) for 16 weeks had reduced % steatotic area compared to mice on Western Diet treated with PBS. Mice treated with the 60 ug dose of ETD02284 (Group 12) had a larger reduction in % steatotic area compared to mice treated with ETD02282.

TABLE 57
16-week % Steatotic Area of Livers in
Mice Treated with ETD02282 and ETD02284

| | | Individual % | Mean % |
|---|---|---|---|
| Group # | Animal # | Steatotic Area | Steatotic Area |
| 8 | 16 | 8.32 | 8.13 |
| | 17 | 10.78 | |
| | 18 | 2.25 | |
| | 19 | 11.37 | |
| | 20 | 7.95 | |
| 9 | 26 | 5.12 | 4.67 |
| | 27 | 4.36 | |
| | 28 | 3.03 | |
| | 29 | 4.35 | |
| | 30 | 6.47 | |
| 10 | 36 | 3.85 | 4.29 |
| | 37 | 5.15 | |
| | 38 | 4.38 | |
| | 39 | 3.65 | |
| | 40 | 4.44 | |
| 11 | 46 | 3.46 | 4.71 |
| | 47 | 5.73 | |
| | 48 | 3.89 | |
| | 49 | 5.58 | |
| | 50 | 4.88 | |
| 12 | 56 | 0.55 | 2.23 |
| | 57 | 3.21 | |
| | 58 | 2.51 | |
| | 59 | 2.34 | |
| | 60 | 2.56 | |

Liver triglycerides were measured in apiece of flash frozen liver using a Triglyceride Colorimetric Kit (Elabscience #E-BC-K238) following the manufacturer's instructions. The levels of triglycerides in the flash frozen liver sections are shown in Table 58 with the data normalized to mice on normal diet treated with PBS (Group 1). The level of triglycerides in the liver at 8 weeks was reduced in mice treated with 200 ug (Group 3) and 60 ug (Group 4) of ETD02282 and 200 ug (Group 5) and 60 ug (Group 6) of ETD02284 (Group 4) compared to mice treated with PBS (Group 2) on Western Diet.

TABLE 58
8-week Liver Triglyceride Levels in Mice
Treated with ETD02282 and ETD02284

| | | Relative Liver | Relative Liver |
|---|---|---|---|
| | | Triglyceride | Triglyceride |
| Group # | Animal # | Level Individual | Level Mean |
| 1 | 1 | 0.77 | 1.00 |
| | 2 | 1.76 | |
| | 3 | 0.95 | |
| | 4 | 0.91 | |
| | 5 | 0.62 | |
| 2 | 11 | 1.46 | 3.82 |
| | 12 | 2.18 | |
| | 13 | 1.35 | |
| | 14 | 1.47 | |
| | 15 | 12.63 | |
| 3 | 21 | 2.12 | 2.34 |
| | 22 | 2.16 | |
| | 23 | 1.88 | |
| | 24 | 2.67 | |
| | 25 | 2.85 | |
| 4 | 31 | 2.58 | 1.77 |
| | 32 | 1.43 | |
| | 33 | 1.64 | |
| | 34 | 1.70 | |
| | 35 | 1.51 | |
| 5 | 41 | 1.63 | 1.72 |
| | 42 | 2.45 | |
| | 43 | 1.64 | |
| | 44 | 1.21 | |
| | 45 | 1.67 | |
| 6 | 51 | 2.80 | 2.30 |
| | 52 | 2.89 | |
| | 53 | 5.54 | |
| | 54 | 0.13 | |
| | 55 | 0.14 | |

Liver triglycerides were measured in apiece of flash frozen liver using a Triglyceride Colorimetric Kit (Elabscience #E-BC-K238) following the manufacturer's instructions. The levels of triglycerides in the flash frozen liver sections are shown in Table 59 with the data normalized to mice on normal diet treated with PBS (Group 7). The level of triglycerides in the liver at 16 weeks were not significantly reduced in mice treated with either dose of ETD02282 (Groups 9 and 10) and 200 ug ETD02284 (Group 11) compared to mice treated with PBS (Group 8) on Western Diet. The samples from the 60 ug dose of ETD02284 (Group 12) could not be successfully processed for this experiment.

TABLE 59
16-week Liver Triglyceride Levels in Mice
Treated with ETD02282 and ETD02284

| | | Relative Liver | Relative Liver |
|---|---|---|---|
| | | Triglyceride | Triglyceride |
| Group # | Animal # | Level Individual | Level Mean |
| 7 | 6 | 0.94 | 1.00 |
| | 7 | 0.82 | |
| | 8 | 0.61 | |
| | 9 | 1.46 | |
| | 10 | 1.17 | |
| 8 | 16 | 1.35 | 1.64 |
| | 17 | 2.48 | |
| | 18 | 1.18 | |
| | 19 | 1.73 | |
| | 20 | 1.46 | |
| 9 | 26 | 1.54 | 1.36 |
| | 27 | 1.45 | |
| | 28 | 1.33 | |
| | 29 | 1.02 | |
| | 30 | 1.44 | |
| 10 | 36 | 1.22 | 1.45 |
| | 37 | 1.71 | |
| | 38 | 1.58 | |
| | 39 | 1.67 | |
| | 40 | 1.06 | |
| 11 | 46 | 2.09 | 2.26 |
| | 47 | 1.27 | |
| | 48 | 3.81 | |
| | 49 | 2.60 | |
| | 50 | 1.52 | |
| 12 | 56 | — | — |
| | 57 | — | |
| | 58 | — | |
| | 59 | — | |
| | 60 | — | |

Expression of fibrotic and pro-inflammatory genes in the liver were measured by qRT-PCR and normalized to the mice on Western Diet receiving PBS (Group 2). The expression of pro-fibrotic genes COL1A1 and TIMP1 and pro-inflammatory genes TNF-α, IL1b, and CCL2 are shown in Table 60. Mice on Western Diet treated with 200 ug ETD02282 (Group 3) showed no reduction of pro-fibrotic or pro-inflammatory genes compared to mice treated with PBS (Group 2). Mice on Western Diet treated with 60 ug of ETD02282 (Group 4) had reduced expression of pro-fibrotic genes COL1A1 and TIMP1 and reduced expression of CCL2 compared to mice treated with PBS (Group 2). Mice on Western Diet treated with 200 ug of ETD02284 (Group 5) and 60 ug (Group 6) had reduced expression of pro-fibrotic genes COL1A1 and TIMP1 and reduced expression of pro-inflammatory genes TNF-alpha, IL1b, and CCL2 when compared to mice treated with PBS (Group 2).

TABLE 60
8-week Expression of Pro-fibrotic and Pro-inflammatory
Genes in Mice Treated with ETD02282 and ETD02284

| | | COL1A1 | | TIMP1 | | TNF-alpha | | IL1b | | CCL2 | |
|---|---|---|---|---|---|---|---|---|---|---|---|
| Group | | Day 56 | | Day 56 | | Day 56 | | Day 56 | | Day 56 | |
| # | Animal # | Individual | Mean | Individual | Mean | Individual | Mean | Individual | Mean | Individual | Mean |
| 1 | 1 | 0.70 | 0.48 | 0.06 | 0.07 | 1.41 | 0.70 | 0.87 | 0.65 | 0.58 | 0.34 |
| | 2 | 0.48 | | 0.11 | | 0.74 | | 0.44 | | 0.32 | |
| | 3 | 1.11 | | 0.04 | | 0.37 | | 0.93 | | 0.41 | |
| | 4 | 0.27 | | 0.16 | | 0.60 | | 0.42 | | 0.25 | |
| | 5 | 0.28 | | 0.05 | | 0.72 | | 0.80 | | 0.24 | |
| 2 | 11 | 0.89 | 1.00 | 0.13 | 1.00 | 1.17 | 1.00 | 0.75 | 1.00 | 0.58 | 1.00 |
| | 12 | 1.45 | | 0.12 | | 0.67 | | 0.47 | | 0.58 | |
| | 13 | 0.72 | | 6.62 | | 2.03 | | 1.35 | | 1.36 | |
| | 14 | 4.28 | | 16.59 | | 1.85 | | 2.14 | | 1.88 | |
| | 15 | 0.25 | | 0.56 | | 0.34 | | 1.02 | | 1.18 | |
| 3 | 21 | 1.16 | 1.43 | 2.75 | 0.78 | 0.78 | 0.83 | 0.60 | 0.99 | 1.06 | 0.76 |
| | 22 | 1.58 | | 1.58 | | 2.49 | | 1.25 | | 0.81 | |
| | 23 | 0.68 | | 0.40 | | 0.67 | | 1.26 | | 0.86 | |
| | 24 | 1.09 | | 0.06 | | 0.34 | | 0.47 | | 0.50 | |
| | 25 | 4.41 | | 2.86 | | 0.88 | | 2.17 | | 0.67 | |
| 4 | 31 | 0.14 | 0.30 | 0.04 | 0.53 | 2.24 | 1.04 | 9.49 | 2.33 | 0.36 | 0.63 |
| | 32 | 2.28 | | 8.21 | | 2.17 | | 2.16 | | 0.74 | |
| | 33 | 1.84 | | 6.80 | | 0.91 | | 1.13 | | 1.01 | |
| | 34 | 0.04 | | 0.27 | | 0.85 | | 3.93 | | 0.54 | |
| | 35 | 0.09 | | 0.07 | | 0.33 | | 0.76 | | 0.71 | |
| 5 | 41 | 0.16 | 0.15 | 0.12 | 0.45 | 2.57 | 0.35 | 2.56 | 0.79 | 0.57 | 0.53 |
| | 42 | 0.01 | | | | 0.22 | | 0.32 | | 0.41 | |
| | 43 | 0.11 | | 0.03 | | 0.22 | | 0.77 | | 0.29 | |
| | 44 | 1.03 | | 13.41 | | 0.71 | | 1.40 | | 1.30 | |
| | 45 | 0.26 | | 0.75 | | 0.06 | | 0.35 | | 0.48 | |
| 6 | 51 | 0.08 | 0.25 | 0.03 | 0.30 | 0.24 | 0.58 | 0.42 | 0.78 | 0.34 | 0.67 |
| | 52 | 0.09 | | 0.20 | | 0.53 | | 0.52 | | 0.61 | |
| | 53 | 0.42 | | 5.37 | | 3.46 | | — | | 1.45 | |
| | 54 | 0.34 | | 0.04 | | 0.14 | | 0.94 | | 0.46 | |
| | 55 | 0.96 | | 1.64 | | 1.07 | | 1.78 | | 1.00 | |

Expression of fibrotic and pro-inflammatory genes in the liver were measured by qRT-PCR and normalized to the mice on Western Diet receiving PBS (Group 2). The expression of pro-fibrotic genes COL1A1 and TIMP1 and pro-inflammatory genes TNF-α, IL1b, and CCL2 are shown in Table 61. Mice on the Western Diet for 16 weeks and treated with 200 ug (Group 9) or 60 ug (Group 10) of ETD02282 or 200 ug of ETD02284 (Group 11) did not show any significant reduction in expression of pro-fibrotic or pro-inflammatory genes compared to mice treated with PBS (Group 8). The mice treated with 60 ug of ETD02284 (Group 12) showed significant reduction of all pro-fibrotic and pro-inflammatory genes measured at 16 weeks compared to mice on PBS (Group 8).

TABLE 61
16-week Expression of Pro-fibrotic and Pro-inflammatory
Genes in Mice Treated with ETD02282 and ETD02284

| | | COL1A1 | | TIMP1 | | TNF-alpha | | IL1b | | CCL2 | |
|---|---|---|---|---|---|---|---|---|---|---|---|
| Group | | Day 112 | | Day 112 | | Day 112 | | Day 112 | | Day 112 | |
| # | Animal # | Individual | Mean | Individual | Mean | Individual | Mean | Individual | Mean | Individual | Mean |
| 7 | 6 | 0.39 | 0.42 | 0.50 | 0.03 | 1.45 | 0.53 | — | 0.40 | 1.26 | 0.89 |
| | 7 | 0.53 | | 0.25 | | 1.15 | | 0.64 | | 0.93 | |
| | 8 | 0.55 | | 0.11 | | 0.69 | | 0.44 | | 1.10 | |
| | 9 | 0.29 | | 0.00 | | 0.11 | | 0.27 | | 0.56 | |
| | 10 | 0.41 | | 0.00 | | 0.34 | | 0.33 | | 0.80 | |
| 8 | 16 | 0.66 | 1.00 | 1.89 | 1.00 | 0.71 | 1.00 | 0.38 | 1.00 | 0.54 | 1.00 |
| | 17 | 2.25 | | 8.51 | | 1.72 | | 0.61 | | 1.34 | |
| | 18 | 0.46 | | 0.03 | | 0.28 | | 0.23 | | 0.74 | |
| | 19 | 1.63 | | 2.13 | | 3.55 | | 4.85 | | 1.51 | |
| | 20 | 0.90 | | 1.16 | | 0.82 | | 3.87 | | 1.24 | |
| 9 | 26 | 0.56 | 1.05 | 1.32 | 1.60 | 0.34 | 0.97 | 0.40 | 1.16 | 0.71 | 1.41 |
| | 27 | 1.30 | | 1.08 | | 5.35 | | 2.05 | | 3.29 | |
| | 28 | 0.64 | | 0.41 | | 0.21 | | 0.22 | | 0.84 | |
| | 29 | 1.65 | | 4.82 | | 2.75 | | 5.11 | | 2.66 | |
| | 30 | 1.64 | | 3.69 | | 0.82 | | 2.32 | | 1.06 | |
| 10 | 36 | 1.07 | 0.76 | 1.51 | 0.50 | 2.07 | 0.75 | 0.85 | 0.72 | 1.10 | 0.92 |
| | 37 | 0.51 | | 0.04 | | 0.41 | | 0.40 | | 0.83 | |
| | 38 | 0.86 | | 2.06 | | 4.21 | | 1.87 | | 1.25 | |
| | 39 | 0.79 | | 0.31 | | 0.65 | | 0.84 | | 1.14 | |
| | 40 | 0.68 | | 0.87 | | 0.10 | | 0.36 | | 0.50 | |
| 11 | 46 | 0.71 | 1.48 | 1.29 | 3.58 | 1.43 | 1.05 | 0.38 | 1.46 | 0.92 | 1.41 |
| | 47 | 1.68 | | 5.88 | | 1.07 | | 0.72 | | 1.11 | |
| | 48 | 2.49 | | 2.21 | | 2.53 | | 3.07 | | 2.35 | |
| | 49 | 3.03 | | 10.81 | | 3.10 | | 3.91 | | 2.86 | |
| | 50 | 0.78 | | 3.23 | | 0.11 | | 1.99 | | 0.83 | |
| 12 | 56 | 0.45 | 0.51 | 0.00 | 0.03 | 0.15 | 0.17 | 0.18 | 0.32 | 0.58 | 0.65 |
| | 57 | 1.17 | | 0.86 | | 0.80 | | 0.26 | | 1.29 | |
| | 58 | 0.79 | | 0.27 | | 0.17 | | 0.27 | | 0.61 | |
| | 59 | 0.31 | | 0.00 | | 0.09 | | 0.46 | | 0.58 | |
| | 60 | 0.28 | | 0.00 | | 0.08 | | 0.59 | | 0.46 | |

### Example 20: Determining the Activity of a siRNA ETD02318 Targeting GPAM in a Single Dose Study in Non-Human Primates

One group of four male cynomolgus monkeys>3 years old was utilized for this study. Monkeys were maintained on normal chow with ad libitum access throughout the study except prior to blood collections in which they were fasted overnight (at least 12 hours). On study Day 0, cynomolgus monkeys were injected subcutaneously (2 mL/kg) with a single dose of 3 mg/kg ETD02318 at a concentration of 1.5 mg/mL. The sequence of the siRNA used are shown in Table 62, where Nf (e.g. Af, Cf, Gf, Tf, or Uf) is a 2′ fluoro-modified nucleoside, n (e.g. a, c, g, t, or u) is a 2′ O-methyl modified nucleoside, and “s” is a phosphorothioate linkage. The base sequences for each siRNA, with and without the 3′ UU extension, are shown in Table 63.

TABLE 62
Example siRNA Sequence

| | SEQ | | SEQ | |
|---|---|---|---|---|
| | ID | Sense Strand Sequence (5′-3′) with | ID | |
| ETD# | NO: | GalNAc moiety | NO: | Antisense Strand Sequence (5′-3′) |
| ETD02318 | 12926 | [ETL17]sauucaCfUfgCfUfagcaagucasusu | 13033 | usGfsacUfuGfcUfagcaGfuGfaAfususu |

TABLE 63
Example siRNA BASE Sequence

| siRNA | SEQ ID | Sense Strand Base | SEQ ID | Antisense Strand Base |
|---|---|---|---|---|
| Name | NO: | Sequence (5′ to 3′) | NO | Sequence (5′ to 3′) |
| ETD02318 | 13224 | AUUCACUGCUAGCAAGUCAUU | 13545 | UGACUUGCUAGCAGUGAAUUU |
| | | Sense Strand Base | | Antisense Strand Base |
| siRNA | SEQ ID | Sequence (5′ to 3′), | SEQ ID | Sequence (5′ to 3′), |
| Name | NO: | without 3′ overhangs | NO | without 3′ overhangs |
| ETD02318 | 13241 | AUUCACUGCUAGCAAGUCA | 13562 | UGACUUGCUAGCAGUGAAU |
| | | Sense Strand Base | | Antisense Strand Base |
| | | Sequence (5′ to 3′), | | Sequence (5′ to 3′), |
| siRNA | SEQ ID | without 3′ overhangs | SEQ ID | without 3′ overhangs |
| Name | NO: | without 3′ A | NO: | without 5′ U |
| ETD02318 | 13258 | AUUCACUGCUAGCAAGUC | 13579 | GACUUGCUAGCAGUGAAU |

Body weights were recorded weekly on Days −8, −2, 7, 14, 21, and 28 of the study. On study Days −8, −2, 7, 14, 21, and 28 whole blood was collected into tubes with no anti-coagulant and centrifuged to obtain serum after clotting. Clinical chemistry for ALT, AST, ALP, DBIL, TBIL, GLU, UREA, CREA, TG, CHOL, TP, GGT, HDL-CH, LDL-CH, and B-HDBH were analyzed.

On study Day −8 and Day 28, a 5 mg liver biopsy was conducted by anesthetizing the animals with Zoletil (1.5-5.0 mg/kg, i.m.) and xylazine (0.5-2.0 mg/kg, i.m.). The liver biopsy was then placed into 10 v/v RNAlater™ Stabilization Solution (Thermo Fisher, Catalog #AM7020) in 20 seconds and stored for 24 hours at 4° C. The RNAlater was then removed and the liver tissue was stored in the freezer until they were shipped to Empirico.

Total liver RNA was prepared by homogenizing the RNAlater liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The levels of liver GPAM mRNA were assessed by RT-qPCR in triplicate on a QuantStudio™ 6 Pro Real-Time PCR System using TaqMan assays for monkey GPAM (ThermoFisher, assay #Mf02878271_m1), and the monkey housekeeping gene GAPDH (ThermoFisher, assay #Mf04392546_g1) using PerfeCTa® qPCRFastMix®, Low ROX™ (VWR, Catalog #101419-222).

The results of the liver biopsy mRNA analysis are summarized in Table 64. Data for each individual was normalized to its Day ~8 liver biopsy mRNA levels using the delta-delta Ct method. A 64% mean reduction in liver GPAM mRNA was observed with a single dose of ETD02318 at Day 28 after injection.

There were no significant changes in body weight or any of the clinical parameters measured during this study.

TABLE 64
GPAM liver mRNA Levels in Monkeys treated
with a single dose of ETD02318

| | | Day −8 | | Day 28 |
|---|---|---|---|---|
| | Day −8 | Mean | Day 28 | Mean |
| | Relative | Relative | Relative | Relative |
| | Liver GPAM | Liver GPAM | Liver GPAM | Liver GPAM |
| Animal # | mRNA Level | mRNA level | mRNA Level | mRNA level |
| 501 | 1.00 | 1.00 | 0.60 | 0.36 |
| 502 | 1.00 | | 0.35 | |
| 503 | 1.00 | | 0.37 | |
| 504 | 1.00 | | 1.1 | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

### IV. SEQUENCE INFORMATION

Some embodiments include one or more nucleic acid sequences in the following tables:

TABLE 65
Sequence Information

| SEQ ID NO: | Description |
|---|---|
| 1-6354 | GPAM siRNA sense strand sequences |
| 6355-12708 | GPAM siRNA antisense strand sequences |
| 12709-12733 | Modified GPAM siRNA sense strand sequences |
| 12734-12758 | Modified GPAM siRNA antisense strand sequences |
| 12759-12866 | Alternatively modified GPAM siRNA sense strand |
| | sequences |
| 12867 | Full-length human GPAM mRNA sequence |
| | (ENST00000348367.9) |
| 12868-12974 | Modified GPAM sense strand sequences |
| 12973-13081 | Modified GPAM antisense sequences |
| 13082-13402 | Unmodified GPAM sense sequences |
| 13403-13723 | Unmodified GPAM antisense sequences |
| 13724-13751 | Modified GPAM sense strand sequences |
| 13752-13723 | Modified GPAM antisense sequences |
| 13724-13751 | Modified GPAM sense strand sequences |
| 13752-13779 | Modified GPAM antisense sequences |

TABLE 66
GPAM siRNA Sequences

| | SEQ ID | | SEQ ID | Antisense strand sequence (5′- |
|---|---|---|---|---|
| siRNA Name | NO: | Sense strand sequence (5′-3′) | NO: | 3′) |
| siRNA 1 | 1 | GCGCCACUGCAGCUGGCAU | 6355 | AUGCCAGCUGCAGUGGCGC |
| siRNA 2 | 2 | CGCCACUGCAGCUGGCAUU | 6356 | AAUGCCAGCUGCAGUGGCG |
| siRNA 3 | 3 | GCCACUGCAGCUGGCAUUG | 6357 | CAAUGCCAGCUGCAGUGGC |
| siRNA 4 | 4 | CCACUGCAGCUGGCAUUGG | 6358 | CCAAUGCCAGCUGCAGUGG |
| siRNA 5 | 5 | CACUGCAGCUGGCAUUGGC | 6359 | GCCAAUGCCAGCUGCAGUG |
| siRNA 6 | 6 | ACUGCAGCUGGCAUUGGCC | 6360 | GGCCAAUGCCAGCUGCAGU |
| siRNA 7 | 7 | CUGCAGCUGGCAUUGGCCG | 6361 | CGGCCAAUGCCAGCUGCAG |
| siRNA 8 | 8 | UGCAGCUGGCAUUGGCCGG | 6362 | CCGGCCAAUGCCAGCUGCA |
| siRNA 9 | 9 | GCAGCUGGCAUUGGCCGGG | 6363 | CCCGGCCAAUGCCAGCUGC |
| siRNA 10 | 10 | CAGCUGGCAUUGGCCGGGA | 6364 | UCCCGGCCAAUGCCAGCUG |
| siRNA 11 | 11 | AGCUGGCAUUGGCCGGGAC | 6365 | GUCCCGGCCAAUGCCAGCU |
| siRNA 12 | 12 | GCUGGCAUUGGCCGGGACU | 6366 | AGUCCCGGCCAAUGCCAGC |
| siRNA 13 | 13 | CUGGCAUUGGCCGGGACUG | 6367 | CAGUCCCGGCCAAUGCCAG |
| siRNA 14 | 14 | UGGCAUUGGCCGGGACUGG | 6368 | CCAGUCCCGGCCAAUGCCA |
| siRNA 15 | 15 | GGCAUUGGCCGGGACUGGA | 6369 | UCCAGUCCCGGCCAAUGCC |
| siRNA 16 | 16 | GCAUUGGCCGGGACUGGAA | 6370 | UUCCAGUCCCGGCCAAUGC |
| siRNA 17 | 17 | CAUUGGCCGGGACUGGAAG | 6371 | CUUCCAGUCCCGGCCAAUG |
| siRNA 18 | 18 | AUUGGCCGGGACUGGAAG | 6372 | ACUUCCAGUCCCGGCCAAU |
| | | U | | |
| siRNA 19 | 19 | UUGGCCGGGACUGGAAGU | 6373 | CACUUCCAGUCCCGGCCAA |
| | | G | | |
| siRNA 20 | 20 | UGGCCGGGACUGGAAGUGC | 6374 | GCACUUCCAGUCCCGGCCA |
| siRNA 21 | 21 | GGCCGGGACUGGAAGUGCG | 6375 | CGCACUUCCAGUCCCGGCC |
| siRNA 22 | 22 | GCCGGGACUGGAAGUGCGG | 6376 | CCGCACUUCCAGUCCCGGC |
| siRNA 23 | 23 | CCGGGACUGGAAGUGCGGG | 6377 | CCCGCACUUCCAGUCCCGG |
| siRNA 24 | 24 | CGGGACUGGAAGUGCGGGC | 6378 | GCCCGCACUUCCAGUCCCG |
| siRNA 25 | 25 | GGGACUGGAAGUGCGGGC | 6379 | AGCCCGCACUUCCAGUCCC |
| | | U | | |
| siRNA 26 | 26 | GGACUGGAAGUGCGGGCU | 6380 | AAGCCCGCACUUCCAGUCC |
| | | U | | |
| siRNA 27 | 27 | GACUGGAAGUGCGGGCUUC | 6381 | GAAGCCCGCACUUCCAGUC |
| siRNA 28 | 28 | ACUGGAAGUGCGGGCUUCU | 6382 | AGAAGCCCGCACUUCCAGU |
| siRNA 29 | 29 | CUGGAAGUGCGGGCUUCUG | 6383 | CAGAAGCCCGCACUUCCAG |
| siRNA 30 | 30 | UGGAAGUGCGGGCUUCUGC | 6384 | GCAGAAGCCCGCACUUCCA |
| siRNA 31 | 31 | GGAAGUGCGGGCUUCUGCA | 6385 | UGCAGAAGCCCGCACUUCC |
| siRNA 32 | 32 | GAAGUGCGGGCUUCUGCAG | 6386 | CUGCAGAAGCCCGCACUUC |
| siRNA 33 | 33 | AAGUGCGGGCUUCUGCAGC | 6387 | GCUGCAGAAGCCCGCACUU |
| siRNA 34 | 34 | AGUGCGGGCUUCUGCAGCA | 6388 | UGCUGCAGAAGCCCGCACU |
| siRNA 35 | 35 | GUGCGGGCUUCUGCAGCAG | 6389 | CUGCUGCAGAAGCCCGCAC |
| siRNA 36 | 36 | UGCGGGCUUCUGCAGCAGC | 6390 | GCUGCUGCAGAAGCCCGCA |
| siRNA 37 | 37 | GCGGGCUUCUGCAGCAGCC | 6391 | GGCUGCUGCAGAAGCCCGC |
| siRNA 38 | 38 | CGGGCUUCUGCAGCAGCCG | 6392 | CGGCUGCUGCAGAAGCCCG |
| siRNA 39 | 39 | GGGCUUCUGCAGCAGCCGA | 6393 | UCGGCUGCUGCAGAAGCCC |
| siRNA 40 | 40 | GGCUUCUGCAGCAGCCGAA | 6394 | UUCGGCUGCUGCAGAAGCC |
| siRNA 41 | 41 | GCUUCUGCAGCAGCCGAAG | 6395 | CUUCGGCUGCUGCAGAAGC |
| siRNA 42 | 42 | CUUCUGCAGCAGCCGAAGC | 6396 | GCUUCGGCUGCUGCAGAAG |
| siRNA 43 | 43 | UUCUGCAGCAGCCGAAGCU | 6397 | AGCUUCGGCUGCUGCAGAA |
| siRNA 44 | 44 | UCUGCAGCAGCCGAAGCUG | 6398 | CAGCUUCGGCUGCUGCAGA |
| siRNA 45 | 45 | CUGCAGCAGCCGAAGCUGG | 6399 | CCAGCUUCGGCUGCUGCAG |
| siRNA 46 | 46 | UGCAGCAGCCGAAGCUGGA | 6400 | UCCAGCUUCGGCUGCUGCA |
| siRNA 47 | 47 | GCAGCAGCCGAAGCUGGAG | 6401 | CUCCAGCUUCGGCUGCUGC |
| siRNA 48 | 48 | CAGCAGCCGAAGCUGGAGC | 6402 | GCUCCAGCUUCGGCUGCUG |
| siRNA 49 | 49 | AGCAGCCGAAGCUGGAGCU | 6403 | AGCUCCAGCUUCGGCUGCU |
| siRNA 50 | 50 | GCAGCCGAAGCUGGAGCUG | 6404 | CAGCUCCAGCUUCGGCUGC |
| siRNA 51 | 51 | CAGCCGAAGCUGGAGCUGC | 6405 | GCAGCUCCAGCUUCGGCUG |
| siRNA 52 | 52 | AGCCGAAGCUGGAGCUGCU | 6406 | AGCAGCUCCAGCUUCGGCU |
| siRNA 53 | 53 | GCCGAAGCUGGAGCUGCUA | 6407 | UAGCAGCUCCAGCUUCGGC |
| siRNA 54 | 54 | CCGAAGCUGGAGCUGCUAG | 6408 | CUAGCAGCUCCAGCUUCGG |
| siRNA 55 | 55 | CGAAGCUGGAGCUGCUAGG | 6409 | CCUAGCAGCUCCAGCUUCG |
| siRNA 56 | 56 | GAAGCUGGAGCUGCUAGG | 6410 | CCCUAGCAGCUCCAGCUUC |
| | | G | | |
| siRNA 57 | 57 | AAGCUGGAGCUGCUAGGGC | 6411 | GCCCUAGCAGCUCCAGCUU |
| siRNA 58 | 58 | AGCUGGAGCUGCUAGGGCA | 6412 | UGCCCUAGCAGCUCCAGCU |
| siRNA 59 | 59 | GCUGGAGCUGCUAGGGCAG | 6413 | CUGCCCUAGCAGCUCCAGC |
| siRNA 60 | 60 | CUGGAGCUGCUAGGGCAGC | 6414 | GCUGCCCUAGCAGCUCCAG |
| siRNA 61 | 61 | UGGAGCUGCUAGGGCAGCA | 6415 | UGCUGCCCUAGCAGCUCCA |
| siRNA 62 | 62 | GGAGCUGCUAGGGCAGCAG | 6416 | CUGCUGCCCUAGCAGCUCC |
| siRNA 63 | 63 | GAGCUGCUAGGGCAGCAGC | 6417 | GCUGCUGCCCUAGCAGCUC |
| siRNA 64 | 64 | AGCUGCUAGGGCAGCAGCG | 6418 | CGCUGCUGCCCUAGCAGCU |
| siRNA 65 | 65 | GCUGCUAGGGCAGCAGCGG | 6419 | CCGCUGCUGCCCUAGCAGC |
| siRNA 66 | 66 | CUGCUAGGGCAGCAGCGGC | 6420 | GCCGCUGCUGCCCUAGCAG |
| siRNA 67 | 67 | UGCUAGGGCAGCAGCGGCU | 6421 | AGCCGCUGCUGCCCUAGCA |
| siRNA 68 | 68 | GCUAGGGCAGCAGCGGCUC | 6422 | GAGCCGCUGCUGCCCUAGC |
| siRNA 69 | 69 | CUAGGGCAGCAGCGGCUCC | 6423 | GGAGCCGCUGCUGCCCUAG |
| siRNA 70 | 70 | UAGGGCAGCAGCGGCUCCC | 6424 | GGGAGCCGCUGCUGCCCUA |
| siRNA 71 | 71 | AGGGCAGCAGCGGCUCCCC | 6425 | GGGGAGCCGCUGCUGCCCU |
| siRNA 72 | 72 | GGGCAGCAGCGGCUCCCCU | 6426 | AGGGGAGCCGCUGCUGCCC |
| siRNA 73 | 73 | GGCAGCAGCGGCUCCCCUG | 6427 | CAGGGGAGCCGCUGCUGCC |
| siRNA 74 | 74 | GCAGCAGCGGCUCCCCUGU | 6428 | ACAGGGGAGCCGCUGCUGC |
| siRNA 75 | 75 | CAGCAGCGGCUCCCCUGUU | 6429 | AACAGGGGAGCCGCUGCUG |
| siRNA 76 | 76 | AGCAGCGGCUCCCCUGUUG | 6430 | CAACAGGGGAGCCGCUGCU |
| siRNA 77 | 77 | GCAGCGGCUCCCCUGUUGU | 6431 | ACAACAGGGGAGCCGCUGC |
| siRNA 78 | 78 | CAGCGGCUCCCCUGUUGUA | 6432 | UACAACAGGGGAGCCGCUG |
| siRNA 79 | 79 | AGCGGCUCCCCUGUUGUAU | 6433 | AUACAACAGGGGAGCCGCU |
| siRNA 80 | 80 | GCGGCUCCCCUGUUGUAUG | 6434 | CAUACAACAGGGGAGCCGC |
| siRNA 81 | 81 | CGGCUCCCCUGUUGUAUGG | 6435 | CCAUACAACAGGGGAGCCG |
| siRNA 82 | 82 | GGCUCCCCUGUUGUAUGGA | 6436 | UCCAUACAACAGGGGAGCC |
| siRNA 83 | 83 | GCUCCCCUGUUGUAUGGAC | 6437 | GUCCAUACAACAGGGGAGC |
| siRNA 84 | 84 | CUCCCCUGUUGUAUGGACA | 6438 | UGUCCAUACAACAGGGGAG |
| siRNA 85 | 85 | UCCCCUGUUGUAUGGACAU | 6439 | AUGUCCAUACAACAGGGGA |
| siRNA 86 | 86 | CCCCUGUUGUAUGGACAUU | 6440 | AAUGUCCAUACAACAGGGG |
| siRNA 87 | 87 | CCCUGUUGUAUGGACAUUC | 6441 | GAAUGUCCAUACAACAGGG |
| siRNA 88 | 88 | CCUGUUGUAUGGACAUUCU | 6442 | AGAAUGUCCAUACAACAGG |
| siRNA 89 | 89 | CUGUUGUAUGGACAUUCU | 6443 | CAGAAUGUCCAUACAACAG |
| | | G | | |
| siRNA 90 | 90 | UGUUGUAUGGACAUUCUG | 6444 | GCAGAAUGUCCAUACAACA |
| | | C | | |
| siRNA 91 | 91 | GUUGUAUGGACAUUCUGC | 6445 | UGCAGAAUGUCCAUACAAC |
| | | A | | |
| siRNA 92 | 92 | UUGUAUGGACAUUCUGCAC | 6446 | GUGCAGAAUGUCCAUACAA |
| siRNA 93 | 93 | UGUAUGGACAUUCUGCACC | 6447 | GGUGCAGAAUGUCCAUACA |
| siRNA 94 | 94 | GUAUGGACAUUCUGCACCC | 6448 | GGGUGCAGAAUGUCCAUAC |
| siRNA 95 | 95 | UAUGGACAUUCUGCACCCG | 6449 | CGGGUGCAGAAUGUCCAUA |
| siRNA 96 | 96 | AUGGACAUUCUGCACCCGA | 6450 | UCGGGUGCAGAAUGUCCAU |
| siRNA 97 | 97 | UGGACAUUCUGCACCCGAA | 6451 | UUCGGGUGCAGAAUGUCCA |
| siRNA 98 | 98 | GGACAUUCUGCACCCGAAA | 6452 | UUUCGGGUGCAGAAUGUCC |
| siRNA 99 | 99 | GACAUUCUGCACCCGAAAC | 6453 | GUUUCGGGUGCAGAAUGU |
| | | | | C |
| siRNA 100 | 100 | ACAUUCUGCACCCGAAACU | 6454 | AGUUUCGGGUGCAGAAUG |
| | | | | U |
| siRNA 101 | 101 | CAUUCUGCACCCGAAACUG | 6455 | CAGUUUCGGGUGCAGAAU |
| | | | | G |
| siRNA 102 | 102 | AUUCUGCACCCGAAACUGA | 6456 | UCAGUUUCGGGUGCAGAA |
| | | | | U |
| siRNA 103 | 103 | UUCUGCACCCGAAACUGAU | 6457 | AUCAGUUUCGGGUGCAGA |
| | | | | A |
| siRNA 104 | 104 | UCUGCACCCGAAACUGAUA | 6458 | UAUCAGUUUCGGGUGCAG |
| | | | | A |
| siRNA 105 | 105 | CUGCACCCGAAACUGAUAG | 6459 | CUAUCAGUUUCGGGUGCAG |
| siRNA 106 | 106 | UGCACCCGAAACUGAUAGC | 6460 | GCUAUCAGUUUCGGGUGCA |
| siRNA 107 | 107 | GCACCCGAAACUGAUAGCU | 6461 | AGCUAUCAGUUUCGGGUGC |
| siRNA 108 | 108 | CACCCGAAACUGAUAGCUG | 6462 | CAGCUAUCAGUUUCGGGUG |
| siRNA 109 | 109 | ACCCGAAACUGAUAGCUGA | 6463 | UCAGCUAUCAGUUUCGGGU |
| siRNA 110 | 110 | CCCGAAACUGAUAGCUGAG | 6464 | CUCAGCUAUCAGUUUCGGG |
| siRNA 111 | 111 | CCGAAACUGAUAGCUGAGU | 6465 | ACUCAGCUAUCAGUUUCGG |
| siRNA 112 | 112 | CGAAACUGAUAGCUGAGUC | 6466 | GACUCAGCUAUCAGUUUCG |
| siRNA 113 | 113 | GAAACUGAUAGCUGAGUCC | 6467 | GGACUCAGCUAUCAGUUUC |
| siRNA 114 | 114 | AAACUGAUAGCUGAGUCCU | 6468 | AGGACUCAGCUAUCAGUUU |
| siRNA 115 | 115 | AACUGAUAGCUGAGUCCUG | 6469 | CAGGACUCAGCUAUCAGUU |
| siRNA 116 | 116 | ACUGAUAGCUGAGUCCUGA | 6470 | UCAGGACUCAGCUAUCAGU |
| siRNA 117 | 117 | CUGAUAGCUGAGUCCUGAA | 6471 | UUCAGGACUCAGCUAUCAG |
| siRNA 118 | 118 | UGAUAGCUGAGUCCUGAA | 6472 | CUUCAGGACUCAGCUAUCA |
| | | G | | |
| siRNA 119 | 119 | GAUAGCUGAGUCCUGAAG | 6473 | ACUUCAGGACUCAGCUAUC |
| | | U | | |
| siRNA 120 | 120 | AUAGCUGAGUCCUGAAGU | 6474 | AACUUCAGGACUCAGCUAU |
| | | U | | |
| siRNA 121 | 121 | UAGCUGAGUCCUGAAGUU | 6475 | AAACUUCAGGACUCAGCUA |
| | | U | | |
| siRNA 122 | 122 | AGCUGAGUCCUGAAGUUU | 6476 | AAAACUUCAGGACUCAGCU |
| | | U | | |
| siRNA 123 | 123 | GCUGAGUCCUGAAGUUUU | 6477 | UAAAACUUCAGGACUCAGC |
| | | A | | |
| siRNA 124 | 124 | CUGAGUCCUGAAGUUUUA | 6478 | AUAAAACUUCAGGACUCAG |
| | | U | | |
| siRNA 125 | 125 | UGAGUCCUGAAGUUUUAU | 6479 | CAUAAAACUUCAGGACUCA |
| | | G | | |
| siRNA 126 | 126 | GAGUCCUGAAGUUUUAUG | 6480 | ACAUAAAACUUCAGGACUC |
| | | U | | |
| siRNA 127 | 127 | AGUCCUGAAGUUUUAUGU | 6481 | AACAUAAAACUUCAGGACU |
| | | U | | |
| siRNA 128 | 128 | GUCCUGAAGUUUUAUGUU | 6482 | UAACAUAAAACUUCAGGAC |
| | | A | | |
| siRNA 129 | 129 | UCCUGAAGUUUUAUGUUA | 6483 | AUAACAUAAAACUUCAGG |
| | | U | | A |
| siRNA 130 | 130 | CCUGAAGUUUUAUGUUAU | 6484 | CAUAACAUAAAACUUCAGG |
| | | G | | |
| siRNA 131 | 131 | CUGAAGUUUUAUGUUAUG | 6485 | UCAUAACAUAAAACUUCAG |
| | | A | | |
| siRNA 132 | 132 | UGAAGUUUUAUGUUAUGA | 6486 | UUCAUAACAUAAAACUUCA |
| | | A | | |
| siRNA 133 | 133 | GAAGUUUUAUGUUAUGAA | 6487 | UUUCAUAACAUAAAACUUC |
| | | A | | |
| siRNA 134 | 134 | AAGUUUUAUGUUAUGAAA | 6488 | GUUUCAUAACAUAAAACU |
| | | C | | U |
| siRNA 135 | 135 | AGUUUUAUGUUAUGAAAC | 6489 | UGUUUCAUAACAUAAAAC |
| | | A | | U |
| siRNA 136 | 136 | GUUUUAUGUUAUGAAACA | 6490 | CUGUUUCAUAACAUAAAAC |
| | | G | | |
| siRNA 137 | 137 | UUUUAUGUUAUGAAACAG | 6491 | UCUGUUUCAUAACAUAAA |
| | | A | | A |
| siRNA 138 | 138 | UUUAUGUUAUGAAACAGA | 6492 | UUCUGUUUCAUAACAUAA |
| | | A | | A |
| siRNA 139 | 139 | UUAUGUUAUGAAACAGAA | 6493 | CUUCUGUUUCAUAACAUAA |
| | | G | | |
| siRNA 140 | 140 | UAUGUUAUGAAACAGAAG | 6494 | UCUUCUGUUUCAUAACAUA |
| | | A | | |
| siRNA 141 | 141 | AUGUUAUGAAACAGAAGA | 6495 | UUCUUCUGUUUCAUAACAU |
| | | A | | |
| siRNA 142 | 142 | UGUUAUGAAACAGAAGAA | 6496 | GUUCUUCUGUUUCAUAACA |
| | | C | | |
| siRNA 143 | 143 | GUUAUGAAACAGAAGAAC | 6497 | AGUUCUUCUGUUUCAUAAC |
| | | U | | |
| siRNA 144 | 144 | UUAUGAAACAGAAGAACU | 6498 | AAGUUCUUCUGUUUCAUA |
| | | U | | A |
| siRNA 145 | 145 | UAUGAAACAGAAGAACUU | 6499 | AAAGUUCUUCUGUUUCAU |
| | | U | | A |
| siRNA 146 | 146 | AUGAAACAGAAGAACUUU | 6500 | GAAAGUUCUUCUGUUUCA |
| | | C | | U |
| siRNA 147 | 147 | UGAAACAGAAGAACUUUC | 6501 | UGAAAGUUCUUCUGUUUC |
| | | A | | A |
| siRNA 148 | 148 | GAAACAGAAGAACUUUCA | 6502 | AUGAAAGUUCUUCUGUUU |
| | | U | | C |
| siRNA 149 | 149 | AAACAGAAGAACUUUCAUC | 6503 | GAUGAAAGUUCUUCUGUU |
| | | | | U |
| siRNA 150 | 150 | AACAGAAGAACUUUCAUCC | 6504 | GGAUGAAAGUUCUUCUGU |
| | | | | U |
| siRNA 151 | 151 | ACAGAAGAACUUUCAUCCC | 6505 | GGGAUGAAAGUUCUUCUG |
| | | | | U |
| siRNA 152 | 152 | CAGAAGAACUUUCAUCCCA | 6506 | UGGGAUGAAAGUUCUUCU |
| | | | | G |
| siRNA 153 | 153 | AGAAGAACUUUCAUCCCAG | 6507 | CUGGGAUGAAAGUUCUUC |
| | | | | U |
| siRNA 154 | 154 | GAAGAACUUUCAUCCCAGC | 6508 | GCUGGGAUGAAAGUUCUU |
| | | | | C |
| siRNA 155 | 155 | AAGAACUUUCAUCCCAGCA | 6509 | UGCUGGGAUGAAAGUUCU |
| | | | | U |
| siRNA 156 | 156 | AGAACUUUCAUCCCAGCAC | 6510 | GUGCUGGGAUGAAAGUUC |
| | | | | U |
| siRNA 157 | 157 | GAACUUUCAUCCCAGCACA | 6511 | UGUGCUGGGAUGAAAGUU |
| | | | | C |
| siRNA 158 | 158 | AACUUUCAUCCCAGCACAU | 6512 | AUGUGCUGGGAUGAAAGU |
| | | | | U |
| siRNA 159 | 159 | ACUUUCAUCCCAGCACAUG | 6513 | CAUGUGCUGGGAUGAAAG |
| | | | | U |
| siRNA 160 | 160 | CUUUCAUCCCAGCACAUGA | 6514 | UCAUGUGCUGGGAUGAAA |
| | | | | G |
| siRNA 161 | 161 | UUUCAUCCCAGCACAUGAU | 6515 | AUCAUGUGCUGGGAUGAA |
| | | | | A |
| siRNA 162 | 162 | UUCAUCCCAGCACAUGAUU | 6516 | AAUCAUGUGCUGGGAUGA |
| | | | | A |
| siRNA 163 | 163 | UCAUCCCAGCACAUGAUUU | 6517 | AAAUCAUGUGCUGGGAUG |
| | | | | A |
| siRNA 164 | 164 | CAUCCCAGCACAUGAUUUG | 6518 | CAAAUCAUGUGCUGGGAU |
| | | | | G |
| siRNA 165 | 165 | AUCCCAGCACAUGAUUUGG | 6519 | CCAAAUCAUGUGCUGGGAU |
| siRNA 166 | 166 | UCCCAGCACAUGAUUUGGG | 6520 | CCCAAAUCAUGUGCUGGGA |
| siRNA 167 | 167 | CCCAGCACAUGAUUUGGGA | 6521 | UCCCAAAUCAUGUGCUGGG |
| siRNA 168 | 168 | CCAGCACAUGAUUUGGGAA | 6522 | UUCCCAAAUCAUGUGCUGG |
| siRNA 169 | 169 | CAGCACAUGAUUUGGGAA | 6523 | AUUCCCAAAUCAUGUGCUG |
| | | U | | |
| siRNA 170 | 170 | AGCACAUGAUUUGGGAAU | 6524 | AAUUCCCAAAUCAUGUGCU |
| | | U | | |
| siRNA 171 | 171 | GCACAUGAUUUGGGAAUU | 6525 | UAAUUCCCAAAUCAUGUGC |
| | | A | | |
| siRNA 172 | 172 | CACAUGAUUUGGGAAUUA | 6526 | GUAAUUCCCAAAUCAUGUG |
| | | C | | |
| siRNA 173 | 173 | ACAUGAUUUGGGAAUUAC | 6527 | UGUAAUUCCCAAAUCAUGU |
| | | A | | |
| siRNA 174 | 174 | CAUGAUUUGGGAAUUACA | 6528 | GUGUAAUUCCCAAAUCAUG |
| | | C | | |
| siRNA 175 | 175 | AUGAUUUGGGAAUUACAC | 6529 | AGUGUAAUUCCCAAAUCAU |
| | | U | | |
| siRNA 176 | 176 | UGAUUUGGGAAUUACACU | 6530 | AAGUGUAAUUCCCAAAUCA |
| | | U | | |
| siRNA 177 | 177 | GAUUUGGGAAUUACACUU | 6531 | AAAGUGUAAUUCCCAAAUC |
| | | U | | |
| siRNA 178 | 178 | AUUUGGGAAUUACACUUU | 6532 | CAAAGUGUAAUUCCCAAAU |
| | | G | | |
| siRNA 179 | 179 | UUUGGGAAUUACACUUUG | 6533 | ACAAAGUGUAAUUCCCAAA |
| | | U | | |
| siRNA 180 | 180 | UUGGGAAUUACACUUUGU | 6534 | CACAAAGUGUAAUUCCCAA |
| | | G | | |
| siRNA 181 | 181 | UGGGAAUUACACUUUGUG | 6535 | UCACAAAGUGUAAUUCCCA |
| | | A | | |
| siRNA 182 | 182 | GGGAAUUACACUUUGUGA | 6536 | GUCACAAAGUGUAAUUCCC |
| | | C | | |
| siRNA 183 | 183 | GGAAUUACACUUUGUGAC | 6537 | UGUCACAAAGUGUAAUUCC |
| | | A | | |
| siRNA 184 | 184 | GAAUUACACUUUGUGACA | 6538 | AUGUCACAAAGUGUAAUU |
| | | U | | C |
| siRNA 185 | 185 | AAUUACACUUUGUGACAU | 6539 | CAUGUCACAAAGUGUAAU |
| | | G | | U |
| siRNA 186 | 186 | AUUACACUUUGUGACAUG | 6540 | CCAUGUCACAAAGUGUAAU |
| | | G | | |
| siRNA 187 | 187 | UUACACUUUGUGACAUGG | 6541 | UCCAUGUCACAAAGUGUAA |
| | | A | | |
| siRNA 188 | 188 | UACACUUUGUGACAUGGA | 6542 | AUCCAUGUCACAAAGUGUA |
| | | U | | |
| siRNA 189 | 189 | ACACUUUGUGACAUGGAU | 6543 | CAUCCAUGUCACAAAGUGU |
| | | G | | |
| siRNA 190 | 190 | CACUUUGUGACAUGGAUG | 6544 | UCAUCCAUGUCACAAAGUG |
| | | A | | |
| siRNA 191 | 191 | ACUUUGUGACAUGGAUGA | 6545 | UUCAUCCAUGUCACAAAGU |
| | | A | | |
| siRNA 192 | 192 | CUUUGUGACAUGGAUGAA | 6546 | AUUCAUCCAUGUCACAAAG |
| | | U | | |
| siRNA 193 | 193 | UUUGUGACAUGGAUGAAU | 6547 | GAUUCAUCCAUGUCACAAA |
| | | C | | |
| siRNA 194 | 194 | UUGUGACAUGGAUGAAUC | 6548 | AGAUUCAUCCAUGUCACAA |
| | | U | | |
| siRNA 195 | 195 | UGUGACAUGGAUGAAUCU | 6549 | CAGAUUCAUCCAUGUCACA |
| | | G | | |
| siRNA 196 | 196 | GUGACAUGGAUGAAUCUG | 6550 | GCAGAUUCAUCCAUGUCAC |
| | | C | | |
| siRNA 197 | 197 | UGACAUGGAUGAAUCUGC | 6551 | UGCAGAUUCAUCCAUGUCA |
| | | A | | |
| siRNA 198 | 198 | GACAUGGAUGAAUCUGCAC | 6552 | GUGCAGAUUCAUCCAUGUC |
| siRNA 199 | 199 | ACAUGGAUGAAUCUGCACU | 6553 | AGUGCAGAUUCAUCCAUGU |
| siRNA 200 | 200 | CAUGGAUGAAUCUGCACUG | 6554 | CAGUGCAGAUUCAUCCAUG |
| siRNA 201 | 201 | AUGGAUGAAUCUGCACUG | 6555 | UCAGUGCAGAUUCAUCCAU |
| | | A | | |
| siRNA 202 | 202 | UGGAUGAAUCUGCACUGAC | 6556 | GUCAGUGCAGAUUCAUCCA |
| siRNA 203 | 203 | GGAUGAAUCUGCACUGACC | 6557 | GGUCAGUGCAGAUUCAUCC |
| siRNA 204 | 204 | GAUGAAUCUGCACUGACCC | 6558 | GGGUCAGUGCAGAUUCAUC |
| siRNA 205 | 205 | AUGAAUCUGCACUGACCCU | 6559 | AGGGUCAGUGCAGAUUCA |
| | | | | U |
| siRNA 206 | 206 | UGAAUCUGCACUGACCCUU | 6560 | AAGGGUCAGUGCAGAUUC |
| | | | | A |
| siRNA 207 | 207 | GAAUCUGCACUGACCCUUG | 6561 | CAAGGGUCAGUGCAGAUUC |
| siRNA 208 | 208 | AAUCUGCACUGACCCUUGG | 6562 | CCAAGGGUCAGUGCAGAUU |
| siRNA 209 | 209 | AUCUGCACUGACCCUUGGU | 6563 | ACCAAGGGUCAGUGCAGAU |
| siRNA 210 | 210 | UCUGCACUGACCCUUGGUA | 6564 | UACCAAGGGUCAGUGCAGA |
| siRNA 211 | 211 | CUGCACUGACCCUUGGUAC | 6565 | GUACCAAGGGUCAGUGCAG |
| siRNA 212 | 212 | UGCACUGACCCUUGGUACA | 6566 | UGUACCAAGGGUCAGUGCA |
| siRNA 213 | 213 | GCACUGACCCUUGGUACAA | 6567 | UUGUACCAAGGGUCAGUGC |
| siRNA 214 | 214 | CACUGACCCUUGGUACAAU | 6568 | AUUGUACCAAGGGUCAGU |
| | | | | G |
| siRNA 215 | 215 | ACUGACCCUUGGUACAAUA | 6569 | UAUUGUACCAAGGGUCAG |
| | | | | U |
| siRNA 216 | 216 | CUGACCCUUGGUACAAUAG | 6570 | CUAUUGUACCAAGGGUCAG |
| siRNA 217 | 217 | UGACCCUUGGUACAAUAGA | 6571 | UCUAUUGUACCAAGGGUCA |
| siRNA 218 | 218 | GACCCUUGGUACAAUAGAU | 6572 | AUCUAUUGUACCAAGGGUC |
| siRNA 219 | 219 | ACCCUUGGUACAAUAGAUG | 6573 | CAUCUAUUGUACCAAGGGU |
| siRNA 220 | 220 | CCCUUGGUACAAUAGAUGU | 6574 | ACAUCUAUUGUACCAAGGG |
| siRNA 221 | 221 | CCUUGGUACAAUAGAUGU | 6575 | AACAUCUAUUGUACCAAGG |
| | | U | | |
| siRNA 222 | 222 | CUUGGUACAAUAGAUGUU | 6576 | AAACAUCUAUUGUACCAAG |
| | | U | | |
| siRNA 223 | 223 | UUGGUACAAUAGAUGUUU | 6577 | GAAACAUCUAUUGUACCAA |
| | | C | | |
| siRNA 224 | 224 | UGGUACAAUAGAUGUUUC | 6578 | AGAAACAUCUAUUGUACCA |
| | | U | | |
| siRNA 225 | 225 | GGUACAAUAGAUGUUUCU | 6579 | AAGAAACAUCUAUUGUACC |
| | | U | | |
| siRNA 226 | 226 | GUACAAUAGAUGUUUCUU | 6580 | UAAGAAACAUCUAUUGUA |
| | | A | | C |
| siRNA 227 | 227 | UACAAUAGAUGUUUCUUA | 6581 | AUAAGAAACAUCUAUUGU |
| | | U | | A |
| siRNA 228 | 228 | ACAAUAGAUGUUUCUUAU | 6582 | GAUAAGAAACAUCUAUUG |
| | | C | | U |
| siRNA 229 | 229 | CAAUAGAUGUUUCUUAUC | 6583 | AGAUAAGAAACAUCUAUU |
| | | U | | G |
| siRNA 230 | 230 | AAUAGAUGUUUCUUAUCU | 6584 | CAGAUAAGAAACAUCUAU |
| | | G | | U |
| siRNA 231 | 231 | AUAGAUGUUUCUUAUCUG | 6585 | GCAGAUAAGAAACAUCUA |
| | | C | | U |
| siRNA 232 | 232 | UAGAUGUUUCUUAUCUGCC | 6586 | GGCAGAUAAGAAACAUCU |
| | | | | A |
| siRNA 233 | 233 | AGAUGUUUCUUAUCUGCCA | 6587 | UGGCAGAUAAGAAACAUC |
| | | | | U |
| siRNA 234 | 234 | GAUGUUUCUUAUCUGCCAC | 6588 | GUGGCAGAUAAGAAACAU |
| | | | | C |
| siRNA 235 | 235 | AUGUUUCUUAUCUGCCACA | 6589 | UGUGGCAGAUAAGAAACA |
| | | | | U |
| siRNA 236 | 236 | UGUUUCUUAUCUGCCACAU | 6590 | AUGUGGCAGAUAAGAAAC |
| | | | | A |
| siRNA 237 | 237 | GUUUCUUAUCUGCCACAUU | 6591 | AAUGUGGCAGAUAAGAAA |
| | | | | C |
| siRNA 238 | 238 | UUUCUUAUCUGCCACAUUC | 6592 | GAAUGUGGCAGAUAAGAA |
| | | | | A |
| siRNA 239 | 239 | UUCUUAUCUGCCACAUUCA | 6593 | UGAAUGUGGCAGAUAAGA |
| | | | | A |
| siRNA 240 | 240 | UCUUAUCUGCCACAUUCAU | 6594 | AUGAAUGUGGCAGAUAAG |
| | | | | A |
| siRNA 241 | 241 | CUUAUCUGCCACAUUCAUC | 6595 | GAUGAAUGUGGCAGAUAA |
| | | | | G |
| siRNA 242 | 242 | UUAUCUGCCACAUUCAUCA | 6596 | UGAUGAAUGUGGCAGAUA |
| | | | | A |
| siRNA 243 | 243 | UAUCUGCCACAUUCAUCAG | 6597 | CUGAUGAAUGUGGCAGAU |
| | | | | A |
| siRNA 244 | 244 | AUCUGCCACAUUCAUCAGA | 6598 | UCUGAUGAAUGUGGCAGA |
| | | | | U |
| siRNA 245 | 245 | UCUGCCACAUUCAUCAGAA | 6599 | UUCUGAUGAAUGUGGCAG |
| | | | | A |
| siRNA 246 | 246 | CUGCCACAUUCAUCAGAAU | 6600 | AUUCUGAUGAAUGUGGCA |
| | | | | G |
| siRNA 247 | 247 | UGCCACAUUCAUCAGAAUA | 6601 | UAUUCUGAUGAAUGUGGC |
| | | | | A |
| siRNA 248 | 248 | GCCACAUUCAUCAGAAUAC | 6602 | GUAUUCUGAUGAAUGUGG |
| | | | | C |
| siRNA 249 | 249 | CCACAUUCAUCAGAAUACA | 6603 | UGUAUUCUGAUGAAUGUG |
| | | | | G |
| siRNA 250 | 250 | CACAUUCAUCAGAAUACAG | 6604 | CUGUAUUCUGAUGAAUGU |
| | | | | G |
| siRNA 251 | 251 | ACAUUCAUCAGAAUACAGU | 6605 | ACUGUAUUCUGAUGAAUG |
| | | | | U |
| siRNA 252 | 252 | CAUUCAUCAGAAUACAGUG | 6606 | CACUGUAUUCUGAUGAAU |
| | | | | G |
| siRNA 253 | 253 | AUUCAUCAGAAUACAGUG | 6607 | ACACUGUAUUCUGAUGAA |
| | | U | | U |
| siRNA 254 | 254 | UUCAUCAGAAUACAGUGU | 6608 | AACACUGUAUUCUGAUGA |
| | | U | | A |
| siRNA 255 | 255 | UCAUCAGAAUACAGUGUU | 6609 | CAACACUGUAUUCUGAUGA |
| | | G | | |
| siRNA 256 | 256 | CAUCAGAAUACAGUGUUG | 6610 | CCAACACUGUAUUCUGAUG |
| | | G | | |
| siRNA 257 | 257 | AUCAGAAUACAGUGUUGG | 6611 | ACCAACACUGUAUUCUGAU |
| | | U | | |
| siRNA 258 | 258 | UCAGAAUACAGUGUUGGU | 6612 | GACCAACACUGUAUUCUGA |
| | | C | | |
| siRNA 259 | 259 | CAGAAUACAGUGUUGGUC | 6613 | CGACCAACACUGUAUUCUG |
| | | G | | |
| siRNA 260 | 260 | AGAAUACAGUGUUGGUCG | 6614 | UCGACCAACACUGUAUUCU |
| | | A | | |
| siRNA 261 | 261 | GAAUACAGUGUUGGUCGA | 6615 | AUCGACCAACACUGUAUUC |
| | | U | | |
| siRNA 262 | 262 | AAUACAGUGUUGGUCGAU | 6616 | CAUCGACCAACACUGUAUU |
| | | G | | |
| siRNA 263 | 263 | AUACAGUGUUGGUCGAUG | 6617 | ACAUCGACCAACACUGUAU |
| | | U | | |
| siRNA 264 | 264 | UACAGUGUUGGUCGAUGU | 6618 | UACAUCGACCAACACUGUA |
| | | A | | |
| siRNA 265 | 265 | ACAGUGUUGGUCGAUGUA | 6619 | UUACAUCGACCAACACUGU |
| | | A | | |
| siRNA 266 | 266 | CAGUGUUGGUCGAUGUAA | 6620 | CUUACAUCGACCAACACUG |
| | | G | | |
| siRNA 267 | 267 | AGUGUUGGUCGAUGUAAG | 6621 | GCUUACAUCGACCAACACU |
| | | C | | |
| siRNA 268 | 268 | GUGUUGGUCGAUGUAAGC | 6622 | UGCUUACAUCGACCAACAC |
| | | A | | |
| siRNA 269 | 269 | UGUUGGUCGAUGUAAGCA | 6623 | GUGCUUACAUCGACCAACA |
| | | C | | |
| siRNA 270 | 270 | GUUGGUCGAUGUAAGCAC | 6624 | UGUGCUUACAUCGACCAAC |
| | | A | | |
| siRNA 271 | 271 | UUGGUCGAUGUAAGCACAC | 6625 | GUGUGCUUACAUCGACCAA |
| siRNA 272 | 272 | UGGUCGAUGUAAGCACACA | 6626 | UGUGUGCUUACAUCGACCA |
| siRNA 273 | 273 | GGUCGAUGUAAGCACACAA | 6627 | UUGUGUGCUUACAUCGACC |
| siRNA 274 | 274 | GUCGAUGUAAGCACACAAG | 6628 | CUUGUGUGCUUACAUCGAC |
| siRNA 275 | 275 | UCGAUGUAAGCACACAAGU | 6629 | ACUUGUGUGCUUACAUCGA |
| siRNA 276 | 276 | CGAUGUAAGCACACAAGUG | 6630 | CACUUGUGUGCUUACAUCG |
| siRNA 277 | 277 | GAUGUAAGCACACAAGUG | 6631 | UCACUUGUGUGCUUACAUC |
| | | A | | |
| siRNA 278 | 278 | AUGUAAGCACACAAGUGA | 6632 | CUCACUUGUGUGCUUACAU |
| | | G | | |
| siRNA 279 | 279 | UGUAAGCACACAAGUGAG | 6633 | CCUCACUUGUGUGCUUACA |
| | | G | | |
| siRNA 280 | 280 | GUAAGCACACAAGUGAGG | 6634 | UCCUCACUUGUGUGCUUAC |
| | | A | | |
| siRNA 281 | 281 | UAAGCACACAAGUGAGGA | 6635 | UUCCUCACUUGUGUGCUUA |
| | | A | | |
| siRNA 282 | 282 | AAGCACACAAGUGAGGAA | 6636 | AUUCCUCACUUGUGUGCUU |
| | | U | | |
| siRNA 283 | 283 | AGCACACAAGUGAGGAAU | 6637 | CAUUCCUCACUUGUGUGCU |
| | | G | | |
| siRNA 284 | 284 | GCACACAAGUGAGGAAUG | 6638 | CCAUUCCUCACUUGUGUGC |
| | | G | | |
| siRNA 285 | 285 | CACACAAGUGAGGAAUGG | 6639 | CCCAUUCCUCACUUGUGUG |
| | | G | | |
| siRNA 286 | 286 | ACACAAGUGAGGAAUGGG | 6640 | CCCCAUUCCUCACUUGUGU |
| | | G | | |
| siRNA 287 | 287 | CACAAGUGAGGAAUGGGG | 6641 | ACCCCAUUCCUCACUUGUG |
| | | U | | |
| siRNA 288 | 288 | ACAAGUGAGGAAUGGGGU | 6642 | CACCCCAUUCCUCACUUGU |
| | | G | | |
| siRNA 289 | 289 | CAAGUGAGGAAUGGGGUG | 6643 | UCACCCCAUUCCUCACUUG |
| | | A | | |
| siRNA 290 | 290 | AAGUGAGGAAUGGGGUGA | 6644 | CUCACCCCAUUCCUCACUU |
| | | G | | |
| siRNA 291 | 291 | AGUGAGGAAUGGGGUGAG | 6645 | ACUCACCCCAUUCCUCACU |
| | | U | | |
| siRNA 292 | 292 | GUGAGGAAUGGGGUGAGU | 6646 | CACUCACCCCAUUCCUCAC |
| | | G | | |
| siRNA 293 | 293 | UGAGGAAUGGGGUGAGUG | 6647 | ACACUCACCCCAUUCCUCA |
| | | U | | |
| siRNA 294 | 294 | GAGGAAUGGGGUGAGUGU | 6648 | CACACUCACCCCAUUCCUC |
| | | G | | |
| siRNA 295 | 295 | AGGAAUGGGGUGAGUGUG | 6649 | CCACACUCACCCCAUUCCU |
| | | G | | |
| siRNA 296 | 296 | GGAAUGGGGUGAGUGUGG | 6650 | GCCACACUCACCCCAUUCC |
| | | C | | |
| siRNA 297 | 297 | GAAUGGGGUGAGUGUGGC | 6651 | AGCCACACUCACCCCAUUC |
| | | U | | |
| siRNA 298 | 298 | AAUGGGGUGAGUGUGGCU | 6652 | AAGCCACACUCACCCCAUU |
| | | U | | |
| siRNA 299 | 299 | AUGGGGUGAGUGUGGCUU | 6653 | AAAGCCACACUCACCCCAU |
| | | U | | |
| siRNA 300 | 300 | UGGGGUGAGUGUGGCUUU | 6654 | UAAAGCCACACUCACCCCA |
| | | A | | |
| siRNA 301 | 301 | GGGGUGAGUGUGGCUUUA | 6655 | CUAAAGCCACACUCACCCC |
| | | G | | |
| siRNA 302 | 302 | GGGUGAGUGUGGCUUUAG | 6656 | UCUAAAGCCACACUCACCC |
| | | A | | |
| siRNA 303 | 303 | GGUGAGUGUGGCUUUAGA | 6657 | GUCUAAAGCCACACUCACC |
| | | C | | |
| siRNA 304 | 304 | GUGAGUGUGGCUUUAGAC | 6658 | GGUCUAAAGCCACACUCAC |
| | | C | | |
| siRNA 305 | 305 | UGAGUGUGGCUUUAGACCC | 6659 | GGGUCUAAAGCCACACUCA |
| siRNA 306 | 306 | GAGUGUGGCUUUAGACCCA | 6660 | UGGGUCUAAAGCCACACUC |
| siRNA 307 | 307 | AGUGUGGCUUUAGACCCAC | 6661 | GUGGGUCUAAAGCCACACU |
| siRNA 308 | 308 | GUGUGGCUUUAGACCCACC | 6662 | GGUGGGUCUAAAGCCACAC |
| siRNA 309 | 309 | UGUGGCUUUAGACCCACCA | 6663 | UGGUGGGUCUAAAGCCACA |
| siRNA 310 | 310 | GUGGCUUUAGACCCACCAU | 6664 | AUGGUGGGUCUAAAGCCAC |
| siRNA 311 | 311 | UGGCUUUAGACCCACCAUC | 6665 | GAUGGUGGGUCUAAAGCC |
| | | | | A |
| siRNA 312 | 312 | GGCUUUAGACCCACCAUCU | 6666 | AGAUGGUGGGUCUAAAGC |
| | | | | C |
| siRNA 313 | 313 | GCUUUAGACCCACCAUCUU | 6667 | AAGAUGGUGGGUCUAAAG |
| | | | | C |
| siRNA 314 | 314 | CUUUAGACCCACCAUCUUC | 6668 | GAAGAUGGUGGGUCUAAA |
| | | | | G |
| siRNA 315 | 315 | UUUAGACCCACCAUCUUCA | 6669 | UGAAGAUGGUGGGUCUAA |
| | | | | A |
| siRNA 316 | 316 | UUAGACCCACCAUCUUCAG | 6670 | CUGAAGAUGGUGGGUCUA |
| | | | | A |
| siRNA 317 | 317 | UAGACCCACCAUCUUCAGA | 6671 | UCUGAAGAUGGUGGGUCU |
| | | | | A |
| siRNA 318 | 318 | AGACCCACCAUCUUCAGAU | 6672 | AUCUGAAGAUGGUGGGUC |
| | | | | U |
| siRNA 319 | 319 | GACCCACCAUCUUCAGAUC | 6673 | GAUCUGAAGAUGGUGGGU |
| | | | | C |
| siRNA 320 | 320 | ACCCACCAUCUUCAGAUCU | 6674 | AGAUCUGAAGAUGGUGGG |
| | | | | U |
| siRNA 321 | 321 | CCCACCAUCUUCAGAUCUG | 6675 | CAGAUCUGAAGAUGGUGG |
| | | | | G |
| siRNA 322 | 322 | CCACCAUCUUCAGAUCUGC | 6676 | GCAGAUCUGAAGAUGGUG |
| | | | | G |
| siRNA 323 | 323 | CACCAUCUUCAGAUCUGCA | 6677 | UGCAGAUCUGAAGAUGGU |
| | | | | G |
| siRNA 324 | 324 | ACCAUCUUCAGAUCUGCAA | 6678 | UUGCAGAUCUGAAGAUGG |
| | | | | U |
| siRNA 325 | 325 | CCAUCUUCAGAUCUGCAAC | 6679 | GUUGCAGAUCUGAAGAUG |
| | | | | G |
| siRNA 326 | 326 | CAUCUUCAGAUCUGCAACU | 6680 | AGUUGCAGAUCUGAAGAU |
| | | | | G |
| siRNA 327 | 327 | AUCUUCAGAUCUGCAACUU | 6681 | AAGUUGCAGAUCUGAAGA |
| | | | | U |
| siRNA 328 | 328 | UCUUCAGAUCUGCAACUUU | 6682 | AAAGUUGCAGAUCUGAAG |
| | | | | A |
| siRNA 329 | 329 | CUUCAGAUCUGCAACUUUA | 6683 | UAAAGUUGCAGAUCUGAA |
| | | | | G |
| siRNA 330 | 330 | UUCAGAUCUGCAACUUUAA | 6684 | UUAAAGUUGCAGAUCUGA |
| | | | | A |
| siRNA 331 | 331 | UCAGAUCUGCAACUUUAAA | 6685 | UUUAAAGUUGCAGAUCUG |
| | | | | A |
| siRNA 332 | 332 | CAGAUCUGCAACUUUAAAA | 6686 | UUUUAAAGUUGCAGAUCU |
| | | | | G |
| siRNA 333 | 333 | AGAUCUGCAACUUUAAAA | 6687 | AUUUUAAAGUUGCAGAUC |
| | | U | | U |
| siRNA 334 | 334 | GAUCUGCAACUUUAAAAU | 6688 | CAUUUUAAAGUUGCAGAU |
| | | G | | C |
| siRNA 335 | 335 | AUCUGCAACUUUAAAAUG | 6689 | CCAUUUUAAAGUUGCAGA |
| | | G | | U |
| siRNA 336 | 336 | UCUGCAACUUUAAAAUGG | 6690 | UCCAUUUUAAAGUUGCAG |
| | | A | | A |
| siRNA 337 | 337 | CUGCAACUUUAAAAUGGA | 6691 | UUCCAUUUUAAAGUUGCA |
| | | A | | G |
| siRNA 338 | 338 | UGCAACUUUAAAAUGGAA | 6692 | UUUCCAUUUUAAAGUUGC |
| | | A | | A |
| siRNA 339 | 339 | GCAACUUUAAAAUGGAAA | 6693 | CUUUCCAUUUUAAAGUUGC |
| | | G | | |
| siRNA 340 | 340 | CAACUUUAAAAUGGAAAG | 6694 | UCUUUCCAUUUUAAAGUU |
| | | A | | G |
| siRNA 341 | 341 | AACUUUAAAAUGGAAAGA | 6695 | UUCUUUCCAUUUUAAAGU |
| | | A | | U |
| siRNA 342 | 342 | ACUUUAAAAUGGAAAGAA | 6696 | UUUCUUUCCAUUUUAAAG |
| | | A | | U |
| siRNA 343 | 343 | CUUUAAAAUGGAAAGAAA | 6697 | CUUUCUUUCCAUUUUAAAG |
| | | G | | |
| siRNA 344 | 344 | UUUAAAAUGGAAAGAAAG | 6698 | GCUUUCUUUCCAUUUUAAA |
| | | C | | |
| siRNA 345 | 345 | UUAAAAUGGAAAGAAAGC | 6699 | GGCUUUCUUUCCAUUUUAA |
| | | C | | |
| siRNA 346 | 346 | UAAAAUGGAAAGAAAGCC | 6700 | AGGCUUUCUUUCCAUUUUA |
| | | U | | |
| siRNA 347 | 347 | AAAAUGGAAAGAAAGCCU | 6701 | UAGGCUUUCUUUCCAUUUU |
| | | A | | |
| siRNA 348 | 348 | AAAUGGAAAGAAAGCCUA | 6702 | UUAGGCUUUCUUUCCAUUU |
| | | A | | |
| siRNA 349 | 349 | AAUGGAAAGAAAGCCUAA | 6703 | AUUAGGCUUUCUUUCCAUU |
| | | U | | |
| siRNA 350 | 350 | AUGGAAAGAAAGCCUAAU | 6704 | CAUUAGGCUUUCUUUCCAU |
| | | G | | |
| siRNA 351 | 351 | UGGAAAGAAAGCCUAAUG | 6705 | UCAUUAGGCUUUCUUUCCA |
| | | A | | |
| siRNA 352 | 352 | GGAAAGAAAGCCUAAUGA | 6706 | CUCAUUAGGCUUUCUUUCC |
| | | G | | |
| siRNA 353 | 353 | GAAAGAAAGCCUAAUGAG | 6707 | ACUCAUUAGGCUUUCUUUC |
| | | U | | |
| siRNA 354 | 354 | AAAGAAAGCCUAAUGAGU | 6708 | GACUCAUUAGGCUUUCUUU |
| | | C | | |
| siRNA 355 | 355 | AAGAAAGCCUAAUGAGUC | 6709 | CGACUCAUUAGGCUUUCUU |
| | | G | | |
| siRNA 356 | 356 | AGAAAGCCUAAUGAGUCG | 6710 | CCGACUCAUUAGGCUUUCU |
| | | G | | |
| siRNA 357 | 357 | GAAAGCCUAAUGAGUCGG | 6711 | UCCGACUCAUUAGGCUUUC |
| | | A | | |
| siRNA 358 | 358 | AAAGCCUAAUGAGUCGGA | 6712 | UUCCGACUCAUUAGGCUUU |
| | | A | | |
| siRNA 359 | 359 | AAGCCUAAUGAGUCGGAA | 6713 | UUUCCGACUCAUUAGGCUU |
| | | A | | |
| siRNA 360 | 360 | AGCCUAAUGAGUCGGAAA | 6714 | UUUUCCGACUCAUUAGGCU |
| | | A | | |
| siRNA 361 | 361 | GCCUAAUGAGUCGGAAAA | 6715 | CUUUUCCGACUCAUUAGGC |
| | | G | | |
| siRNA 362 | 362 | CCUAAUGAGUCGGAAAAG | 6716 | CCUUUUCCGACUCAUUAGG |
| | | G | | |
| siRNA 363 | 363 | CUAAUGAGUCGGAAAAGG | 6717 | GCCUUUUCCGACUCAUUAG |
| | | C | | |
| siRNA 364 | 364 | UAAUGAGUCGGAAAAGGC | 6718 | GGCCUUUUCCGACUCAUUA |
| | | C | | |
| siRNA 365 | 365 | AAUGAGUCGGAAAAGGCC | 6719 | UGGCCUUUUCCGACUCAUU |
| | | A | | |
| siRNA 366 | 366 | AUGAGUCGGAAAAGGCCA | 6720 | AUGGCCUUUUCCGACUCAU |
| | | U | | |
| siRNA 367 | 367 | UGAGUCGGAAAAGGCCAU | 6721 | AAUGGCCUUUUCCGACUCA |
| | | U | | |
| siRNA 368 | 368 | GAGUCGGAAAAGGCCAUU | 6722 | AAAUGGCCUUUUCCGACUC |
| | | U | | |
| siRNA 369 | 369 | AGUCGGAAAAGGCCAUUU | 6723 | CAAAUGGCCUUUUCCGACU |
| | | G | | |
| siRNA 370 | 370 | GUCGGAAAAGGCCAUUUG | 6724 | ACAAAUGGCCUUUUCCGAC |
| | | U | | |
| siRNA 371 | 371 | UCGGAAAAGGCCAUUUGU | 6725 | AACAAAUGGCCUUUUCCGA |
| | | U | | |
| siRNA 372 | 372 | CGGAAAAGGCCAUUUGUU | 6726 | CAACAAAUGGCCUUUUCCG |
| | | G | | |
| siRNA 373 | 373 | GGAAAAGGCCAUUUGUUG | 6727 | CCAACAAAUGGCCUUUUCC |
| | | G | | |
| siRNA 374 | 374 | GAAAAGGCCAUUUGUUGG | 6728 | UCCAACAAAUGGCCUUUUC |
| | | A | | |
| siRNA 375 | 375 | AAAAGGCCAUUUGUUGGA | 6729 | UUCCAACAAAUGGCCUUUU |
| | | A | | |
| siRNA 376 | 376 | AAAGGCCAUUUGUUGGAA | 6730 | CUUCCAACAAAUGGCCUUU |
| | | G | | |
| siRNA 377 | 377 | AAGGCCAUUUGUUGGAAG | 6731 | UCUUCCAACAAAUGGCCUU |
| | | A | | |
| siRNA 378 | 378 | AGGCCAUUUGUUGGAAGA | 6732 | AUCUUCCAACAAAUGGCCU |
| | | U | | |
| siRNA 379 | 379 | GGCCAUUUGUUGGAAGAU | 6733 | CAUCUUCCAACAAAUGGCC |
| | | G | | |
| siRNA 380 | 380 | GCCAUUUGUUGGAAGAUG | 6734 | ACAUCUUCCAACAAAUGGC |
| | | U | | |
| siRNA 381 | 381 | CCAUUUGUUGGAAGAUGU | 6735 | AACAUCUUCCAACAAAUGG |
| | | U | | |
| siRNA 382 | 382 | CAUUUGUUGGAAGAUGUU | 6736 | CAACAUCUUCCAACAAAUG |
| | | G | | |
| siRNA 383 | 383 | AUUUGUUGGAAGAUGUUG | 6737 | ACAACAUCUUCCAACAAAU |
| | | U | | |
| siRNA 384 | 384 | UUUGUUGGAAGAUGUUGU | 6738 | AACAACAUCUUCCAACAAA |
| | | U | | |
| siRNA 385 | 385 | UUGUUGGAAGAUGUUGUU | 6739 | UAACAACAUCUUCCAACAA |
| | | A | | |
| siRNA 386 | 386 | UGUUGGAAGAUGUUGUUA | 6740 | GUAACAACAUCUUCCAACA |
| | | C | | |
| siRNA 387 | 387 | GUUGGAAGAUGUUGUUAC | 6741 | AGUAACAACAUCUUCCAAC |
| | | U | | |
| siRNA 388 | 388 | UUGGAAGAUGUUGUUACU | 6742 | GAGUAACAACAUCUUCCAA |
| | | C | | |
| siRNA 389 | 389 | UGGAAGAUGUUGUUACUC | 6743 | GGAGUAACAACAUCUUCCA |
| | | C | | |
| siRNA 390 | 390 | GGAAGAUGUUGUUACUCC | 6744 | AGGAGUAACAACAUCUUCC |
| | | U | | |
| siRNA 391 | 391 | GAAGAUGUUGUUACUCCU | 6745 | CAGGAGUAACAACAUCUUC |
| | | G | | |
| siRNA 392 | 392 | AAGAUGUUGUUACUCCUGC | 6746 | GCAGGAGUAACAACAUCUU |
| siRNA 393 | 393 | AGAUGUUGUUACUCCUGCA | 6747 | UGCAGGAGUAACAACAUCU |
| siRNA 394 | 394 | GAUGUUGUUACUCCUGCAC | 6748 | GUGCAGGAGUAACAACAUC |
| siRNA 395 | 395 | AUGUUGUUACUCCUGCACU | 6749 | AGUGCAGGAGUAACAACA |
| | | | | U |
| siRNA 396 | 396 | UGUUGUUACUCCUGCACUC | 6750 | GAGUGCAGGAGUAACAAC |
| | | | | A |
| siRNA 397 | 397 | GUUGUUACUCCUGCACUCC | 6751 | GGAGUGCAGGAGUAACAA |
| | | | | C |
| siRNA 398 | 398 | UUGUUACUCCUGCACUCCC | 6752 | GGGAGUGCAGGAGUAACA |
| | | | | A |
| siRNA 399 | 399 | UGUUACUCCUGCACUCCCC | 6753 | GGGGAGUGCAGGAGUAAC |
| | | | | A |
| siRNA 400 | 400 | GUUACUCCUGCACUCCCCA | 6754 | UGGGGAGUGCAGGAGUAA |
| | | | | C |
| siRNA 401 | 401 | UUACUCCUGCACUCCCCAG | 6755 | CUGGGGAGUGCAGGAGUA |
| | | | | A |
| siRNA 402 | 402 | UACUCCUGCACUCCCCAGA | 6756 | UCUGGGGAGUGCAGGAGU |
| | | | | A |
| siRNA 403 | 403 | ACUCCUGCACUCCCCAGAG | 6757 | CUCUGGGGAGUGCAGGAG |
| | | | | U |
| siRNA 404 | 404 | CUCCUGCACUCCCCAGAGC | 6758 | GCUCUGGGGAGUGCAGGA |
| | | | | G |
| siRNA 405 | 405 | UCCUGCACUCCCCAGAGCU | 6759 | AGCUCUGGGGAGUGCAGG |
| | | | | A |
| siRNA 406 | 406 | CCUGCACUCCCCAGAGCUG | 6760 | CAGCUCUGGGGAGUGCAGG |
| siRNA 407 | 407 | CUGCACUCCCCAGAGCUGG | 6761 | CCAGCUCUGGGGAGUGCAG |
| siRNA 408 | 408 | UGCACUCCCCAGAGCUGGG | 6762 | CCCAGCUCUGGGGAGUGCA |
| siRNA 409 | 409 | GCACUCCCCAGAGCUGGGA | 6763 | UCCCAGCUCUGGGGAGUGC |
| siRNA 410 | 410 | CACUCCCCAGAGCUGGGAC | 6764 | GUCCCAGCUCUGGGGAGUG |
| siRNA 411 | 411 | ACUCCCCAGAGCUGGGACA | 6765 | UGUCCCAGCUCUGGGGAGU |
| siRNA 412 | 412 | CUCCCCAGAGCUGGGACAA | 6766 | UUGUCCCAGCUCUGGGGAG |
| siRNA 413 | 413 | UCCCCAGAGCUGGGACAAA | 6767 | UUUGUCCCAGCUCUGGGGA |
| siRNA 414 | 414 | CCCCAGAGCUGGGACAAAU | 6768 | AUUUGUCCCAGCUCUGGGG |
| siRNA 415 | 415 | CCCAGAGCUGGGACAAAUU | 6769 | AAUUUGUCCCAGCUCUGGG |
| siRNA 416 | 416 | CCAGAGCUGGGACAAAUUU | 6770 | AAAUUUGUCCCAGCUCUGG |
| siRNA 417 | 417 | CAGAGCUGGGACAAAUUU | 6771 | AAAAUUUGUCCCAGCUCUG |
| | | U | | |
| siRNA 418 | 418 | AGAGCUGGGACAAAUUUU | 6772 | AAAAAUUUGUCCCAGCUCU |
| | | U | | |
| siRNA 419 | 419 | GAGCUGGGACAAAUUUUU | 6773 | GAAAAAUUUGUCCCAGCUC |
| | | C | | |
| siRNA 420 | 420 | AGCUGGGACAAAUUUUUC | 6774 | UGAAAAAUUUGUCCCAGCU |
| | | A | | |
| siRNA 421 | 421 | GCUGGGACAAAUUUUUCA | 6775 | UUGAAAAAUUUGUCCCAGC |
| | | A | | |
| siRNA 422 | 422 | CUGGGACAAAUUUUUCAAC | 6776 | GUUGAAAAAUUUGUCCCA |
| | | | | G |
| siRNA 423 | 423 | UGGGACAAAUUUUUCAACC | 6777 | GGUUGAAAAAUUUGUCCC |
| | | | | A |
| siRNA 424 | 424 | GGGACAAAUUUUUCAACCC | 6778 | GGGUUGAAAAAUUUGUCC |
| | | | | C |
| siRNA 425 | 425 | GGACAAAUUUUUCAACCCC | 6779 | GGGGUUGAAAAAUUUGUC |
| | | | | C |
| siRNA 426 | 426 | GACAAAUUUUUCAACCCCA | 6780 | UGGGGUUGAAAAAUUUGU |
| | | | | C |
| siRNA 427 | 427 | ACAAAUUUUUCAACCCCAG | 6781 | CUGGGGUUGAAAAAUUUG |
| | | | | U |
| siRNA 428 | 428 | CAAAUUUUUCAACCCCAGU | 6782 | ACUGGGGUUGAAAAAUUU |
| | | | | G |
| siRNA 429 | 429 | AAAUUUUUCAACCCCAGUA | 6783 | UACUGGGGUUGAAAAAUU |
| | | | | U |
| siRNA 430 | 430 | AAUUUUUCAACCCCAGUAU | 6784 | AUACUGGGGUUGAAAAAU |
| | | | | U |
| siRNA 431 | 431 | AUUUUUCAACCCCAGUAUC | 6785 | GAUACUGGGGUUGAAAAA |
| | | | | U |
| siRNA 432 | 432 | UUUUUCAACCCCAGUAUCC | 6786 | GGAUACUGGGGUUGAAAA |
| | | | | A |
| siRNA 433 | 433 | UUUUCAACCCCAGUAUCCC | 6787 | GGGAUACUGGGGUUGAAA |
| | | | | A |
| siRNA 434 | 434 | UUUCAACCCCAGUAUCCCG | 6788 | CGGGAUACUGGGGUUGAA |
| | | | | A |
| siRNA 435 | 435 | UUCAACCCCAGUAUCCCGU | 6789 | ACGGGAUACUGGGGUUGA |
| | | | | A |
| siRNA 436 | 436 | UCAACCCCAGUAUCCCGUC | 6790 | GACGGGAUACUGGGGUUG |
| | | | | A |
| siRNA 437 | 437 | CAACCCCAGUAUCCCGUCU | 6791 | AGACGGGAUACUGGGGUU |
| | | | | G |
| siRNA 438 | 438 | AACCCCAGUAUCCCGUCUU | 6792 | AAGACGGGAUACUGGGGU |
| | | | | U |
| siRNA 439 | 439 | ACCCCAGUAUCCCGUCUUU | 6793 | AAAGACGGGAUACUGGGG |
| | | | | U |
| siRNA 440 | 440 | CCCCAGUAUCCCGUCUUUG | 6794 | CAAAGACGGGAUACUGGG |
| | | | | G |
| siRNA 441 | 441 | CCCAGUAUCCCGUCUUUGG | 6795 | CCAAAGACGGGAUACUGGG |
| siRNA 442 | 442 | CCAGUAUCCCGUCUUUGGG | 6796 | CCCAAAGACGGGAUACUGG |
| siRNA 443 | 443 | CAGUAUCCCGUCUUUGGGU | 6797 | ACCCAAAGACGGGAUACUG |
| siRNA 444 | 444 | AGUAUCCCGUCUUUGGGUU | 6798 | AACCCAAAGACGGGAUACU |
| siRNA 445 | 445 | GUAUCCCGUCUUUGGGUUU | 6799 | AAACCCAAAGACGGGAUAC |
| siRNA 446 | 446 | UAUCCCGUCUUUGGGUUUG | 6800 | CAAACCCAAAGACGGGAUA |
| siRNA 447 | 447 | AUCCCGUCUUUGGGUUUGC | 6801 | GCAAACCCAAAGACGGGAU |
| siRNA 448 | 448 | UCCCGUCUUUGGGUUUGCG | 6802 | CGCAAACCCAAAGACGGGA |
| siRNA 449 | 449 | CCCGUCUUUGGGUUUGCGG | 6803 | CCGCAAACCCAAAGACGGG |
| siRNA 450 | 450 | CCGUCUUUGGGUUUGCGGA | 6804 | UCCGCAAACCCAAAGACGG |
| siRNA 451 | 451 | CGUCUUUGGGUUUGCGGA | 6805 | UUCCGCAAACCCAAAGACG |
| | | A | | |
| siRNA 452 | 452 | GUCUUUGGGUUUGCGGAA | 6806 | AUUCCGCAAACCCAAAGAC |
| | | U | | |
| siRNA 453 | 453 | UCUUUGGGUUUGCGGAAU | 6807 | CAUUCCGCAAACCCAAAGA |
| | | G | | |
| siRNA 454 | 454 | CUUUGGGUUUGCGGAAUG | 6808 | ACAUUCCGCAAACCCAAAG |
| | | U | | |
| siRNA 455 | 455 | UUUGGGUUUGCGGAAUGU | 6809 | AACAUUCCGCAAACCCAAA |
| | | U | | |
| siRNA 456 | 456 | UUGGGUUUGCGGAAUGUU | 6810 | UAACAUUCCGCAAACCCAA |
| | | A | | |
| siRNA 457 | 457 | UGGGUUUGCGGAAUGUUA | 6811 | AUAACAUUCCGCAAACCCA |
| | | U | | |
| siRNA 458 | 458 | GGGUUUGCGGAAUGUUAU | 6812 | AAUAACAUUCCGCAAACCC |
| | | U | | |
| siRNA 459 | 459 | GGUUUGCGGAAUGUUAUU | 6813 | AAAUAACAUUCCGCAAACC |
| | | U | | |
| siRNA 460 | 460 | GUUUGCGGAAUGUUAUUU | 6814 | UAAAUAACAUUCCGCAAAC |
| | | A | | |
| siRNA 461 | 461 | UUUGCGGAAUGUUAUUUA | 6815 | AUAAAUAACAUUCCGCAAA |
| | | U | | |
| siRNA 462 | 462 | UUGCGGAAUGUUAUUUAU | 6816 | UAUAAAUAACAUUCCGCAA |
| | | A | | |
| siRNA 463 | 463 | UGCGGAAUGUUAUUUAUA | 6817 | AUAUAAAUAACAUUCCGCA |
| | | U | | |
| siRNA 464 | 464 | GCGGAAUGUUAUUUAUAU | 6818 | GAUAUAAAUAACAUUCCGC |
| | | C | | |
| siRNA 465 | 465 | CGGAAUGUUAUUUAUAUC | 6819 | UGAUAUAAAUAACAUUCC |
| | | A | | G |
| siRNA 466 | 466 | GGAAUGUUAUUUAUAUCA | 6820 | UUGAUAUAAAUAACAUUC |
| | | A | | C |
| siRNA 467 | 467 | GAAUGUUAUUUAUAUCAA | 6821 | AUUGAUAUAAAUAACAUU |
| | | U | | C |
| siRNA 468 | 468 | AAUGUUAUUUAUAUCAAU | 6822 | CAUUGAUAUAAAUAACAU |
| | | G | | U |
| siRNA 469 | 469 | AUGUUAUUUAUAUCAAUG | 6823 | UCAUUGAUAUAAAUAACA |
| | | A | | U |
| siRNA 470 | 470 | UGUUAUUUAUAUCAAUGA | 6824 | UUCAUUGAUAUAAAUAAC |
| | | A | | A |
| siRNA 471 | 471 | GUUAUUUAUAUCAAUGAA | 6825 | UUUCAUUGAUAUAAAUAA |
| | | A | | C |
| siRNA 472 | 472 | UUAUUUAUAUCAAUGAAA | 6826 | GUUUCAUUGAUAUAAAUA |
| | | C | | A |
| siRNA 473 | 473 | UAUUUAUAUCAAUGAAAC | 6827 | AGUUUCAUUGAUAUAAAU |
| | | U | | A |
| siRNA 474 | 474 | AUUUAUAUCAAUGAAACU | 6828 | GAGUUUCAUUGAUAUAAA |
| | | C | | U |
| siRNA 475 | 475 | UUUAUAUCAAUGAAACUC | 6829 | UGAGUUUCAUUGAUAUAA |
| | | A | | A |
| siRNA 476 | 476 | UUAUAUCAAUGAAACUCAC | 6830 | GUGAGUUUCAUUGAUAUA |
| | | | | A |
| siRNA 477 | 477 | UAUAUCAAUGAAACUCACA | 6831 | UGUGAGUUUCAUUGAUAU |
| | | | | A |
| siRNA 478 | 478 | AUAUCAAUGAAACUCACAC | 6832 | GUGUGAGUUUCAUUGAUA |
| | | | | U |
| siRNA 479 | 479 | UAUCAAUGAAACUCACACA | 6833 | UGUGUGAGUUUCAUUGAU |
| | | | | A |
| siRNA 480 | 480 | AUCAAUGAAACUCACACAA | 6834 | UUGUGUGAGUUUCAUUGA |
| | | | | U |
| siRNA 481 | 481 | UCAAUGAAACUCACACAAG | 6835 | CUUGUGUGAGUUUCAUUG |
| | | | | A |
| siRNA 482 | 482 | CAAUGAAACUCACACAAGA | 6836 | UCUUGUGUGAGUUUCAUU |
| | | | | G |
| siRNA 483 | 483 | AAUGAAACUCACACAAGAC | 6837 | GUCUUGUGUGAGUUUCAU |
| | | | | U |
| siRNA 484 | 484 | AUGAAACUCACACAAGACA | 6838 | UGUCUUGUGUGAGUUUCA |
| | | | | U |
| siRNA 485 | 485 | UGAAACUCACACAAGACAC | 6839 | GUGUCUUGUGUGAGUUUC |
| | | | | A |
| siRNA 486 | 486 | GAAACUCACACAAGACACC | 6840 | GGUGUCUUGUGUGAGUUU |
| | | | | C |
| siRNA 487 | 487 | AAACUCACACAAGACACCG | 6841 | CGGUGUCUUGUGUGAGUU |
| | | | | U |
| siRNA 488 | 488 | AACUCACACAAGACACCGC | 6842 | GCGGUGUCUUGUGUGAGU |
| | | | | U |
| siRNA 489 | 489 | ACUCACACAAGACACCGCG | 6843 | CGCGGUGUCUUGUGUGAG |
| | | | | U |
| siRNA 490 | 490 | CUCACACAAGACACCGCGG | 6844 | CCGCGGUGUCUUGUGUGAG |
| siRNA 491 | 491 | UCACACAAGACACCGCGGA | 6845 | UCCGCGGUGUCUUGUGUGA |
| siRNA 492 | 492 | CACACAAGACACCGCGGAU | 6846 | AUCCGCGGUGUCUUGUGUG |
| siRNA 493 | 493 | ACACAAGACACCGCGGAUG | 6847 | CAUCCGCGGUGUCUUGUGU |
| siRNA 494 | 494 | CACAAGACACCGCGGAUGG | 6848 | CCAUCCGCGGUGUCUUGUG |
| siRNA 495 | 495 | ACAAGACACCGCGGAUGGC | 6849 | GCCAUCCGCGGUGUCUUGU |
| siRNA 496 | 496 | CAAGACACCGCGGAUGGCU | 6850 | AGCCAUCCGCGGUGUCUUG |
| siRNA 497 | 497 | AAGACACCGCGGAUGGCUU | 6851 | AAGCCAUCCGCGGUGUCUU |
| siRNA 498 | 498 | AGACACCGCGGAUGGCUUG | 6852 | CAAGCCAUCCGCGGUGUCU |
| siRNA 499 | 499 | GACACCGCGGAUGGCUUGC | 6853 | GCAAGCCAUCCGCGGUGUC |
| siRNA 500 | 500 | ACACCGCGGAUGGCUUGCA | 6854 | UGCAAGCCAUCCGCGGUGU |
| siRNA 501 | 501 | CACCGCGGAUGGCUUGCAA | 6855 | UUGCAAGCCAUCCGCGGUG |
| siRNA 502 | 502 | ACCGCGGAUGGCUUGCAAG | 6856 | CUUGCAAGCCAUCCGCGGU |
| siRNA 503 | 503 | CCGCGGAUGGCUUGCAAGA | 6857 | UCUUGCAAGCCAUCCGCGG |
| siRNA 504 | 504 | CGCGGAUGGCUUGCAAGAC | 6858 | GUCUUGCAAGCCAUCCGCG |
| siRNA 505 | 505 | GCGGAUGGCUUGCAAGACG | 6859 | CGUCUUGCAAGCCAUCCGC |
| siRNA 506 | 506 | CGGAUGGCUUGCAAGACGC | 6860 | GCGUCUUGCAAGCCAUCCG |
| siRNA 507 | 507 | GGAUGGCUUGCAAGACGCC | 6861 | GGCGUCUUGCAAGCCAUCC |
| siRNA 508 | 508 | GAUGGCUUGCAAGACGCCU | 6862 | AGGCGUCUUGCAAGCCAUC |
| siRNA 509 | 509 | AUGGCUUGCAAGACGCCUU | 6863 | AAGGCGUCUUGCAAGCCAU |
| siRNA 510 | 510 | UGGCUUGCAAGACGCCUUU | 6864 | AAAGGCGUCUUGCAAGCCA |
| siRNA 511 | 511 | GGCUUGCAAGACGCCUUUC | 6865 | GAAAGGCGUCUUGCAAGCC |
| siRNA 512 | 512 | GCUUGCAAGACGCCUUUCU | 6866 | AGAAAGGCGUCUUGCAAGC |
| siRNA 513 | 513 | CUUGCAAGACGCCUUUCUU | 6867 | AAGAAAGGCGUCUUGCAA |
| | | | | G |
| siRNA 514 | 514 | UUGCAAGACGCCUUUCUUA | 6868 | UAAGAAAGGCGUCUUGCA |
| | | | | A |
| siRNA 515 | 515 | UGCAAGACGCCUUUCUUAC | 6869 | GUAAGAAAGGCGUCUUGC |
| | | | | A |
| siRNA 516 | 516 | GCAAGACGCCUUUCUUACG | 6870 | CGUAAGAAAGGCGUCUUGC |
| siRNA 517 | 517 | CAAGACGCCUUUCUUACGU | 6871 | ACGUAAGAAAGGCGUCUU |
| | | | | G |
| siRNA 518 | 518 | AAGACGCCUUUCUUACGUU | 6872 | AACGUAAGAAAGGCGUCU |
| | | | | U |
| siRNA 519 | 519 | AGACGCCUUUCUUACGUUC | 6873 | GAACGUAAGAAAGGCGUC |
| | | | | U |
| siRNA 520 | 520 | GACGCCUUUCUUACGUUCU | 6874 | AGAACGUAAGAAAGGCGU |
| | | | | C |
| siRNA 521 | 521 | ACGCCUUUCUUACGUUCUU | 6875 | AAGAACGUAAGAAAGGCG |
| | | | | U |
| siRNA 522 | 522 | CGCCUUUCUUACGUUCUUU | 6876 | AAAGAACGUAAGAAAGGC |
| | | | | G |
| siRNA 523 | 523 | GCCUUUCUUACGUUCUUUU | 6877 | AAAAGAACGUAAGAAAGG |
| | | | | C |
| siRNA 524 | 524 | CCUUUCUUACGUUCUUUUU | 6878 | AAAAAGAACGUAAGAAAG |
| | | | | G |
| siRNA 525 | 525 | CUUUCUUACGUUCUUUUUA | 6879 | UAAAAAGAACGUAAGAAA |
| | | | | G |
| siRNA 526 | 526 | UUUCUUACGUUCUUUUUA | 6880 | AUAAAAAGAACGUAAGAA |
| | | U | | A |
| siRNA 527 | 527 | UUCUUACGUUCUUUUUAU | 6881 | AAUAAAAAGAACGUAAGA |
| | | U | | A |
| siRNA 528 | 528 | UCUUACGUUCUUUUUAUUC | 6882 | GAAUAAAAAGAACGUAAG |
| | | | | A |
| siRNA 529 | 529 | CUUACGUUCUUUUUAUUCA | 6883 | UGAAUAAAAAGAACGUAA |
| | | | | G |
| siRNA 530 | 530 | UUACGUUCUUUUUAUUCA | 6884 | UUGAAUAAAAAGAACGUA |
| | | A | | A |
| siRNA 531 | 531 | UACGUUCUUUUUAUUCAA | 6885 | CUUGAAUAAAAAGAACGU |
| | | G | | A |
| siRNA 532 | 532 | ACGUUCUUUUUAUUCAAG | 6886 | UCUUGAAUAAAAAGAACG |
| | | A | | U |
| siRNA 533 | 533 | CGUUCUUUUUAUUCAAGA | 6887 | CUCUUGAAUAAAAAGAAC |
| | | G | | G |
| siRNA 534 | 534 | GUUCUUUUUAUUCAAGAG | 6888 | GCUCUUGAAUAAAAAGAA |
| | | C | | C |
| siRNA 535 | 535 | UUCUUUUUAUUCAAGAGC | 6889 | CGCUCUUGAAUAAAAAGA |
| | | G | | A |
| siRNA 536 | 536 | UCUUUUUAUUCAAGAGCG | 6890 | UCGCUCUUGAAUAAAAAG |
| | | A | | A |
| siRNA 537 | 537 | CUUUUUAUUCAAGAGCGA | 6891 | CUCGCUCUUGAAUAAAAAG |
| | | G | | |
| siRNA 538 | 538 | UUUUUAUUCAAGAGCGAG | 6892 | UCUCGCUCUUGAAUAAAAA |
| | | A | | |
| siRNA 539 | 539 | UUUUAUUCAAGAGCGAGA | 6893 | AUCUCGCUCUUGAAUAAAA |
| | | U | | |
| siRNA 540 | 540 | UUUAUUCAAGAGCGAGAU | 6894 | CAUCUCGCUCUUGAAUAAA |
| | | G | | |
| siRNA 541 | 541 | UUAUUCAAGAGCGAGAUG | 6895 | ACAUCUCGCUCUUGAAUAA |
| | | U | | |
| siRNA 542 | 542 | UAUUCAAGAGCGAGAUGU | 6896 | CACAUCUCGCUCUUGAAUA |
| | | G | | |
| siRNA 543 | 543 | AUUCAAGAGCGAGAUGUG | 6897 | GCACAUCUCGCUCUUGAAU |
| | | C | | |
| siRNA 544 | 544 | UUCAAGAGCGAGAUGUGC | 6898 | UGCACAUCUCGCUCUUGAA |
| | | A | | |
| siRNA 545 | 545 | UCAAGAGCGAGAUGUGCA | 6899 | AUGCACAUCUCGCUCUUGA |
| | | U | | |
| siRNA 546 | 546 | CAAGAGCGAGAUGUGCAU | 6900 | UAUGCACAUCUCGCUCUUG |
| | | A | | |
| siRNA 547 | 547 | AAGAGCGAGAUGUGCAUA | 6901 | UUAUGCACAUCUCGCUCUU |
| | | A | | |
| siRNA 548 | 548 | AGAGCGAGAUGUGCAUAA | 6902 | CUUAUGCACAUCUCGCUCU |
| | | G | | |
| siRNA 549 | 549 | GAGCGAGAUGUGCAUAAG | 6903 | CCUUAUGCACAUCUCGCUC |
| | | G | | |
| siRNA 550 | 550 | AGCGAGAUGUGCAUAAGG | 6904 | CCCUUAUGCACAUCUCGCU |
| | | G | | |
| siRNA 551 | 551 | GCGAGAUGUGCAUAAGGG | 6905 | GCCCUUAUGCACAUCUCGC |
| | | C | | |
| siRNA 552 | 552 | CGAGAUGUGCAUAAGGGC | 6906 | UGCCCUUAUGCACAUCUCG |
| | | A | | |
| siRNA 553 | 553 | GAGAUGUGCAUAAGGGCA | 6907 | AUGCCCUUAUGCACAUCUC |
| | | U | | |
| siRNA 554 | 554 | AGAUGUGCAUAAGGGCAU | 6908 | CAUGCCCUUAUGCACAUCU |
| | | G | | |
| siRNA 555 | 555 | GAUGUGCAUAAGGGCAUG | 6909 | ACAUGCCCUUAUGCACAUC |
| | | U | | |
| siRNA 556 | 556 | AUGUGCAUAAGGGCAUGU | 6910 | AACAUGCCCUUAUGCACAU |
| | | U | | |
| siRNA 557 | 557 | UGUGCAUAAGGGCAUGUU | 6911 | AAACAUGCCCUUAUGCACA |
| | | U | | |
| siRNA 558 | 558 | GUGCAUAAGGGCAUGUUU | 6912 | CAAACAUGCCCUUAUGCAC |
| | | G | | |
| siRNA 559 | 559 | UGCAUAAGGGCAUGUUUG | 6913 | GCAAACAUGCCCUUAUGCA |
| | | C | | |
| siRNA 560 | 560 | GCAUAAGGGCAUGUUUGCC | 6914 | GGCAAACAUGCCCUUAUGC |
| siRNA 561 | 561 | CAUAAGGGCAUGUUUGCCA | 6915 | UGGCAAACAUGCCCUUAUG |
| siRNA 562 | 562 | AUAAGGGCAUGUUUGCCAC | 6916 | GUGGCAAACAUGCCCUUAU |
| siRNA 563 | 563 | UAAGGGCAUGUUUGCCACC | 6917 | GGUGGCAAACAUGCCCUUA |
| siRNA 564 | 564 | AAGGGCAUGUUUGCCACCA | 6918 | UGGUGGCAAACAUGCCCUU |
| siRNA 565 | 565 | AGGGCAUGUUUGCCACCAA | 6919 | UUGGUGGCAAACAUGCCCU |
| siRNA 566 | 566 | GGGCAUGUUUGCCACCAAU | 6920 | AUUGGUGGCAAACAUGCCC |
| siRNA 567 | 567 | GGCAUGUUUGCCACCAAUG | 6921 | CAUUGGUGGCAAACAUGCC |
| siRNA 568 | 568 | GCAUGUUUGCCACCAAUGU | 6922 | ACAUUGGUGGCAAACAUGC |
| siRNA 569 | 569 | CAUGUUUGCCACCAAUGUG | 6923 | CACAUUGGUGGCAAACAUG |
| siRNA 570 | 570 | AUGUUUGCCACCAAUGUGA | 6924 | UCACAUUGGUGGCAAACAU |
| siRNA 571 | 571 | UGUUUGCCACCAAUGUGAC | 6925 | GUCACAUUGGUGGCAAACA |
| siRNA 572 | 572 | GUUUGCCACCAAUGUGACU | 6926 | AGUCACAUUGGUGGCAAAC |
| siRNA 573 | 573 | UUUGCCACCAAUGUGACUG | 6927 | CAGUCACAUUGGUGGCAAA |
| siRNA 574 | 574 | UUGCCACCAAUGUGACUGA | 6928 | UCAGUCACAUUGGUGGCAA |
| siRNA 575 | 575 | UGCCACCAAUGUGACUGAA | 6929 | UUCAGUCACAUUGGUGGCA |
| siRNA 576 | 576 | GCCACCAAUGUGACUGAAA | 6930 | UUUCAGUCACAUUGGUGGC |
| siRNA 577 | 577 | CCACCAAUGUGACUGAAAA | 6931 | UUUUCAGUCACAUUGGUG |
| | | | | G |
| siRNA 578 | 578 | CACCAAUGUGACUGAAAAU | 6932 | AUUUUCAGUCACAUUGGU |
| | | | | G |
| siRNA 579 | 579 | ACCAAUGUGACUGAAAAU | 6933 | CAUUUUCAGUCACAUUGGU |
| | | G | | |
| siRNA 580 | 580 | CCAAUGUGACUGAAAAUG | 6934 | ACAUUUUCAGUCACAUUGG |
| | | U | | |
| siRNA 581 | 581 | CAAUGUGACUGAAAAUGU | 6935 | CACAUUUUCAGUCACAUUG |
| | | G | | |
| siRNA 582 | 582 | AAUGUGACUGAAAAUGUG | 6936 | GCACAUUUUCAGUCACAUU |
| | | C | | |
| siRNA 583 | 583 | AUGUGACUGAAAAUGUGC | 6937 | AGCACAUUUUCAGUCACAU |
| | | U | | |
| siRNA 584 | 584 | UGUGACUGAAAAUGUGCU | 6938 | CAGCACAUUUUCAGUCACA |
| | | G | | |
| siRNA 585 | 585 | GUGACUGAAAAUGUGCUG | 6939 | UCAGCACAUUUUCAGUCAC |
| | | A | | |
| siRNA 586 | 586 | UGACUGAAAAUGUGCUGA | 6940 | UUCAGCACAUUUUCAGUCA |
| | | A | | |
| siRNA 587 | 587 | GACUGAAAAUGUGCUGAA | 6941 | GUUCAGCACAUUUUCAGUC |
| | | C | | |
| siRNA 588 | 588 | ACUGAAAAUGUGCUGAAC | 6942 | UGUUCAGCACAUUUUCAGU |
| | | A | | |
| siRNA 589 | 589 | CUGAAAAUGUGCUGAACA | 6943 | CUGUUCAGCACAUUUUCAG |
| | | G | | |
| siRNA 590 | 590 | UGAAAAUGUGCUGAACAG | 6944 | GCUGUUCAGCACAUUUUCA |
| | | C | | |
| siRNA 591 | 591 | GAAAAUGUGCUGAACAGC | 6945 | UGCUGUUCAGCACAUUUUC |
| | | A | | |
| siRNA 592 | 592 | AAAAUGUGCUGAACAGCA | 6946 | CUGCUGUUCAGCACAUUUU |
| | | G | | |
| siRNA 593 | 593 | AAAUGUGCUGAACAGCAG | 6947 | ACUGCUGUUCAGCACAUUU |
| | | U | | |
| siRNA 594 | 594 | AAUGUGCUGAACAGCAGU | 6948 | UACUGCUGUUCAGCACAUU |
| | | A | | |
| siRNA 595 | 595 | AUGUGCUGAACAGCAGUA | 6949 | CUACUGCUGUUCAGCACAU |
| | | G | | |
| siRNA 596 | 596 | UGUGCUGAACAGCAGUAG | 6950 | UCUACUGCUGUUCAGCACA |
| | | A | | |
| siRNA 597 | 597 | GUGCUGAACAGCAGUAGA | 6951 | CUCUACUGCUGUUCAGCAC |
| | | G | | |
| siRNA 598 | 598 | UGCUGAACAGCAGUAGAG | 6952 | ACUCUACUGCUGUUCAGCA |
| | | U | | |
| siRNA 599 | 599 | GCUGAACAGCAGUAGAGU | 6953 | UACUCUACUGCUGUUCAGC |
| | | A | | |
| siRNA 600 | 600 | CUGAACAGCAGUAGAGUAC | 6954 | GUACUCUACUGCUGUUCAG |
| siRNA 601 | 601 | UGAACAGCAGUAGAGUAC | 6955 | UGUACUCUACUGCUGUUCA |
| | | A | | |
| siRNA 602 | 602 | GAACAGCAGUAGAGUACA | 6956 | UUGUACUCUACUGCUGUUC |
| | | A | | |
| siRNA 603 | 603 | AACAGCAGUAGAGUACAA | 6957 | CUUGUACUCUACUGCUGUU |
| | | G | | |
| siRNA 604 | 604 | ACAGCAGUAGAGUACAAG | 6958 | UCUUGUACUCUACUGCUGU |
| | | A | | |
| siRNA 605 | 605 | CAGCAGUAGAGUACAAGA | 6959 | CUCUUGUACUCUACUGCUG |
| | | G | | |
| siRNA 606 | 606 | AGCAGUAGAGUACAAGAG | 6960 | CCUCUUGUACUCUACUGCU |
| | | G | | |
| siRNA 607 | 607 | GCAGUAGAGUACAAGAGG | 6961 | GCCUCUUGUACUCUACUGC |
| | | C | | |
| siRNA 608 | 608 | CAGUAGAGUACAAGAGGC | 6962 | UGCCUCUUGUACUCUACUG |
| | | A | | |
| siRNA 609 | 609 | AGUAGAGUACAAGAGGCA | 6963 | UUGCCUCUUGUACUCUACU |
| | | A | | |
| siRNA 610 | 610 | GUAGAGUACAAGAGGCAA | 6964 | AUUGCCUCUUGUACUCUAC |
| | | U | | |
| siRNA 611 | 611 | UAGAGUACAAGAGGCAAU | 6965 | AAUUGCCUCUUGUACUCUA |
| | | U | | |
| siRNA 612 | 612 | AGAGUACAAGAGGCAAUU | 6966 | CAAUUGCCUCUUGUACUCU |
| | | G | | |
| siRNA 613 | 613 | GAGUACAAGAGGCAAUUG | 6967 | GCAAUUGCCUCUUGUACUC |
| | | C | | |
| siRNA 614 | 614 | AGUACAAGAGGCAAUUGC | 6968 | UGCAAUUGCCUCUUGUACU |
| | | A | | |
| siRNA 615 | 615 | GUACAAGAGGCAAUUGCA | 6969 | CUGCAAUUGCCUCUUGUAC |
| | | G | | |
| siRNA 616 | 616 | UACAAGAGGCAAUUGCAG | 6970 | UCUGCAAUUGCCUCUUGUA |
| | | A | | |
| siRNA 617 | 617 | ACAAGAGGCAAUUGCAGA | 6971 | UUCUGCAAUUGCCUCUUGU |
| | | A | | |
| siRNA 618 | 618 | CAAGAGGCAAUUGCAGAA | 6972 | CUUCUGCAAUUGCCUCUUG |
| | | G | | |
| siRNA 619 | 619 | AAGAGGCAAUUGCAGAAG | 6973 | ACUUCUGCAAUUGCCUCUU |
| | | U | | |
| siRNA 620 | 620 | AGAGGCAAUUGCAGAAGU | 6974 | CACUUCUGCAAUUGCCUCU |
| | | G | | |
| siRNA 621 | 621 | GAGGCAAUUGCAGAAGUG | 6975 | CCACUUCUGCAAUUGCCUC |
| | | G | | |
| siRNA 622 | 622 | AGGCAAUUGCAGAAGUGG | 6976 | GCCACUUCUGCAAUUGCCU |
| | | C | | |
| siRNA 623 | 623 | GGCAAUUGCAGAAGUGGC | 6977 | AGCCACUUCUGCAAUUGCC |
| | | U | | |
| siRNA 624 | 624 | GCAAUUGCAGAAGUGGCU | 6978 | CAGCCACUUCUGCAAUUGC |
| | | G | | |
| siRNA 625 | 625 | CAAUUGCAGAAGUGGCUGC | 6979 | GCAGCCACUUCUGCAAUUG |
| siRNA 626 | 626 | AAUUGCAGAAGUGGCUGC | 6980 | AGCAGCCACUUCUGCAAUU |
| | | U | | |
| siRNA 627 | 627 | AUUGCAGAAGUGGCUGCU | 6981 | CAGCAGCCACUUCUGCAAU |
| | | G | | |
| siRNA 628 | 628 | UUGCAGAAGUGGCUGCUG | 6982 | UCAGCAGCCACUUCUGCAA |
| | | A | | |
| siRNA 629 | 629 | UGCAGAAGUGGCUGCUGA | 6983 | UUCAGCAGCCACUUCUGCA |
| | | A | | |
| siRNA 630 | 630 | GCAGAAGUGGCUGCUGAA | 6984 | AUUCAGCAGCCACUUCUGC |
| | | U | | |
| siRNA 631 | 631 | CAGAAGUGGCUGCUGAAU | 6985 | AAUUCAGCAGCCACUUCUG |
| | | U | | |
| siRNA 632 | 632 | AGAAGUGGCUGCUGAAUU | 6986 | UAAUUCAGCAGCCACUUCU |
| | | A | | |
| siRNA 633 | 633 | GAAGUGGCUGCUGAAUUA | 6987 | UUAAUUCAGCAGCCACUUC |
| | | A | | |
| siRNA 634 | 634 | AAGUGGCUGCUGAAUUAA | 6988 | UUUAAUUCAGCAGCCACUU |
| | | A | | |
| siRNA 635 | 635 | AGUGGCUGCUGAAUUAAA | 6989 | GUUUAAUUCAGCAGCCACU |
| | | C | | |
| siRNA 636 | 636 | GUGGCUGCUGAAUUAAACC | 6990 | GGUUUAAUUCAGCAGCCAC |
| siRNA 637 | 637 | UGGCUGCUGAAUUAAACCC | 6991 | GGGUUUAAUUCAGCAGCCA |
| siRNA 638 | 638 | GGCUGCUGAAUUAAACCCU | 6992 | AGGGUUUAAUUCAGCAGCC |
| siRNA 639 | 639 | GCUGCUGAAUUAAACCCUG | 6993 | CAGGGUUUAAUUCAGCAGC |
| siRNA 640 | 640 | CUGCUGAAUUAAACCCUGA | 6994 | UCAGGGUUUAAUUCAGCA |
| | | | | G |
| siRNA 641 | 641 | UGCUGAAUUAAACCCUGAU | 6995 | AUCAGGGUUUAAUUCAGC |
| | | | | A |
| siRNA 642 | 642 | GCUGAAUUAAACCCUGAUG | 6996 | CAUCAGGGUUUAAUUCAGC |
| siRNA 643 | 643 | CUGAAUUAAACCCUGAUGG | 6997 | CCAUCAGGGUUUAAUUCAG |
| siRNA 644 | 644 | UGAAUUAAACCCUGAUGG | 6998 | ACCAUCAGGGUUUAAUUCA |
| | | U | | |
| siRNA 645 | 645 | GAAUUAAACCCUGAUGGU | 6999 | AACCAUCAGGGUUUAAUUC |
| | | U | | |
| siRNA 646 | 646 | AAUUAAACCCUGAUGGUUC | 7000 | GAACCAUCAGGGUUUAAU |
| | | | | U |
| siRNA 647 | 647 | AUUAAACCCUGAUGGUUCU | 7001 | AGAACCAUCAGGGUUUAA |
| | | | | U |
| siRNA 648 | 648 | UUAAACCCUGAUGGUUCUG | 7002 | CAGAACCAUCAGGGUUUAA |
| siRNA 649 | 649 | UAAACCCUGAUGGUUCUGC | 7003 | GCAGAACCAUCAGGGUUUA |
| siRNA 650 | 650 | AAACCCUGAUGGUUCUGCC | 7004 | GGCAGAACCAUCAGGGUUU |
| siRNA 651 | 651 | AACCCUGAUGGUUCUGCCC | 7005 | GGGCAGAACCAUCAGGGUU |
| siRNA 652 | 652 | ACCCUGAUGGUUCUGCCCA | 7006 | UGGGCAGAACCAUCAGGGU |
| siRNA 653 | 653 | CCCUGAUGGUUCUGCCCAG | 7007 | CUGGGCAGAACCAUCAGGG |
| siRNA 654 | 654 | CCUGAUGGUUCUGCCCAGC | 7008 | GCUGGGCAGAACCAUCAGG |
| siRNA 655 | 655 | CUGAUGGUUCUGCCCAGCA | 7009 | UGCUGGGCAGAACCAUCAG |
| siRNA 656 | 656 | UGAUGGUUCUGCCCAGCAG | 7010 | CUGCUGGGCAGAACCAUCA |
| siRNA 657 | 657 | GAUGGUUCUGCCCAGCAGC | 7011 | GCUGCUGGGCAGAACCAUC |
| siRNA 658 | 658 | AUGGUUCUGCCCAGCAGCA | 7012 | UGCUGCUGGGCAGAACCAU |
| siRNA 659 | 659 | UGGUUCUGCCCAGCAGCAA | 7013 | UUGCUGCUGGGCAGAACCA |
| siRNA 660 | 660 | GGUUCUGCCCAGCAGCAAU | 7014 | AUUGCUGCUGGGCAGAACC |
| siRNA 661 | 661 | GUUCUGCCCAGCAGCAAUC | 7015 | GAUUGCUGCUGGGCAGAAC |
| siRNA 662 | 662 | UUCUGCCCAGCAGCAAUCA | 7016 | UGAUUGCUGCUGGGCAGA |
| | | | | A |
| siRNA 663 | 663 | UCUGCCCAGCAGCAAUCAA | 7017 | UUGAUUGCUGCUGGGCAG |
| | | | | A |
| siRNA 664 | 664 | CUGCCCAGCAGCAAUCAAA | 7018 | UUUGAUUGCUGCUGGGCA |
| | | | | G |
| siRNA 665 | 665 | UGCCCAGCAGCAAUCAAAA | 7019 | UUUUGAUUGCUGCUGGGC |
| | | | | A |
| siRNA 666 | 666 | GCCCAGCAGCAAUCAAAAG | 7020 | CUUUUGAUUGCUGCUGGGC |
| siRNA 667 | 667 | CCCAGCAGCAAUCAAAAGC | 7021 | GCUUUUGAUUGCUGCUGG |
| | | | | G |
| siRNA 668 | 668 | CCAGCAGCAAUCAAAAGCC | 7022 | GGCUUUUGAUUGCUGCUG |
| | | | | G |
| siRNA 669 | 669 | CAGCAGCAAUCAAAAGCCG | 7023 | CGGCUUUUGAUUGCUGCUG |
| siRNA 670 | 670 | AGCAGCAAUCAAAAGCCGU | 7024 | ACGGCUUUUGAUUGCUGCU |
| siRNA 671 | 671 | GCAGCAAUCAAAAGCCGUU | 7025 | AACGGCUUUUGAUUGCUGC |
| siRNA 672 | 672 | CAGCAAUCAAAAGCCGUUA | 7026 | UAACGGCUUUUGAUUGCU |
| | | | | G |
| siRNA 673 | 673 | AGCAAUCAAAAGCCGUUAA | 7027 | UUAACGGCUUUUGAUUGC |
| | | | | U |
| siRNA 674 | 674 | GCAAUCAAAAGCCGUUAAC | 7028 | GUUAACGGCUUUUGAUUG |
| | | | | C |
| siRNA 675 | 675 | CAAUCAAAAGCCGUUAACA | 7029 | UGUUAACGGCUUUUGAUU |
| | | | | G |
| siRNA 676 | 676 | AAUCAAAAGCCGUUAACAA | 7030 | UUGUUAACGGCUUUUGAU |
| | | | | U |
| siRNA 677 | 677 | AUCAAAAGCCGUUAACAAA | 7031 | UUUGUUAACGGCUUUUGA |
| | | | | U |
| siRNA 678 | 678 | UCAAAAGCCGUUAACAAAG | 7032 | CUUUGUUAACGGCUUUUG |
| | | | | A |
| siRNA 679 | 679 | CAAAAGCCGUUAACAAAGU | 7033 | ACUUUGUUAACGGCUUUU |
| | | | | G |
| siRNA 680 | 680 | AAAAGCCGUUAACAAAGU | 7034 | CACUUUGUUAACGGCUUUU |
| | | G | | |
| siRNA 681 | 681 | AAAGCCGUUAACAAAGUG | 7035 | UCACUUUGUUAACGGCUUU |
| | | A | | |
| siRNA 682 | 682 | AAGCCGUUAACAAAGUGA | 7036 | UUCACUUUGUUAACGGCUU |
| | | A | | |
| siRNA 683 | 683 | AGCCGUUAACAAAGUGAA | 7037 | UUUCACUUUGUUAACGGCU |
| | | A | | |
| siRNA 684 | 684 | GCCGUUAACAAAGUGAAA | 7038 | UUUUCACUUUGUUAACGGC |
| | | A | | |
| siRNA 685 | 685 | CCGUUAACAAAGUGAAAA | 7039 | UUUUUCACUUUGUUAACG |
| | | A | | G |
| siRNA 686 | 686 | CGUUAACAAAGUGAAAAA | 7040 | CUUUUUCACUUUGUUAACG |
| | | G | | |
| siRNA 687 | 687 | GUUAACAAAGUGAAAAAG | 7041 | UCUUUUUCACUUUGUUAAC |
| | | A | | |
| siRNA 688 | 688 | UUAACAAAGUGAAAAAGA | 7042 | UUCUUUUUCACUUUGUUA |
| | | A | | A |
| siRNA 689 | 689 | UAACAAAGUGAAAAAGAA | 7043 | UUUCUUUUUCACUUUGUU |
| | | A | | A |
| siRNA 690 | 690 | AACAAAGUGAAAAAGAAA | 7044 | CUUUCUUUUUCACUUUGUU |
| | | G | | |
| siRNA 691 | 691 | ACAAAGUGAAAAAGAAAG | 7045 | GCUUUCUUUUUCACUUUGU |
| | | C | | |
| siRNA 692 | 692 | CAAAGUGAAAAAGAAAGC | 7046 | AGCUUUCUUUUUCACUUUG |
| | | U | | |
| siRNA 693 | 693 | AAAGUGAAAAAGAAAGCU | 7047 | UAGCUUUCUUUUUCACUUU |
| | | A | | |
| siRNA 694 | 694 | AAGUGAAAAAGAAAGCUA | 7048 | UUAGCUUUCUUUUUCACUU |
| | | A | | |
| siRNA 695 | 695 | AGUGAAAAAGAAAGCUAA | 7049 | UUUAGCUUUCUUUUUCACU |
| | | A | | |
| siRNA 696 | 696 | GUGAAAAAGAAAGCUAAA | 7050 | UUUUAGCUUUCUUUUUCAC |
| | | A | | |
| siRNA 697 | 697 | UGAAAAAGAAAGCUAAAA | 7051 | CUUUUAGCUUUCUUUUUCA |
| | | G | | |
| siRNA 698 | 698 | GAAAAAGAAAGCUAAAAG | 7052 | CCUUUUAGCUUUCUUUUUC |
| | | G | | |
| siRNA 699 | 699 | AAAAAGAAAGCUAAAAGG | 7053 | UCCUUUUAGCUUUCUUUUU |
| | | A | | |
| siRNA 700 | 700 | AAAAGAAAGCUAAAAGGA | 7054 | AUCCUUUUAGCUUUCUUUU |
| | | U | | |
| siRNA 701 | 701 | AAAGAAAGCUAAAAGGAU | 7055 | AAUCCUUUUAGCUUUCUUU |
| | | U | | |
| siRNA 702 | 702 | AAGAAAGCUAAAAGGAUU | 7056 | GAAUCCUUUUAGCUUUCUU |
| | | C | | |
| siRNA 703 | 703 | AGAAAGCUAAAAGGAUUC | 7057 | AGAAUCCUUUUAGCUUUCU |
| | | U | | |
| siRNA 704 | 704 | GAAAGCUAAAAGGAUUCU | 7058 | AAGAAUCCUUUUAGCUUUC |
| | | U | | |
| siRNA 705 | 705 | AAAGCUAAAAGGAUUCUU | 7059 | GAAGAAUCCUUUUAGCUU |
| | | C | | U |
| siRNA 706 | 706 | AAGCUAAAAGGAUUCUUC | 7060 | UGAAGAAUCCUUUUAGCU |
| | | A | | U |
| siRNA 707 | 707 | AGCUAAAAGGAUUCUUCA | 7061 | UUGAAGAAUCCUUUUAGC |
| | | A | | U |
| siRNA 708 | 708 | GCUAAAAGGAUUCUUCAA | 7062 | CUUGAAGAAUCCUUUUAGC |
| | | G | | |
| siRNA 709 | 709 | CUAAAAGGAUUCUUCAAG | 7063 | UCUUGAAGAAUCCUUUUA |
| | | A | | G |
| siRNA 710 | 710 | UAAAAGGAUUCUUCAAGA | 7064 | UUCUUGAAGAAUCCUUUU |
| | | A | | A |
| siRNA 711 | 711 | AAAAGGAUUCUUCAAGAA | 7065 | UUUCUUGAAGAAUCCUUU |
| | | A | | U |
| siRNA 712 | 712 | AAAGGAUUCUUCAAGAAA | 7066 | AUUUCUUGAAGAAUCCUU |
| | | U | | U |
| siRNA 713 | 713 | AAGGAUUCUUCAAGAAAU | 7067 | CAUUUCUUGAAGAAUCCUU |
| | | G | | |
| siRNA 714 | 714 | AGGAUUCUUCAAGAAAUG | 7068 | CCAUUUCUUGAAGAAUCCU |
| | | G | | |
| siRNA 715 | 715 | GGAUUCUUCAAGAAAUGG | 7069 | ACCAUUUCUUGAAGAAUCC |
| | | U | | |
| siRNA 716 | 716 | GAUUCUUCAAGAAAUGGU | 7070 | AACCAUUUCUUGAAGAAUC |
| | | U | | |
| siRNA 717 | 717 | AUUCUUCAAGAAAUGGUU | 7071 | CAACCAUUUCUUGAAGAAU |
| | | G | | |
| siRNA 718 | 718 | UUCUUCAAGAAAUGGUUG | 7072 | GCAACCAUUUCUUGAAGAA |
| | | C | | |
| siRNA 719 | 719 | UCUUCAAGAAAUGGUUGCC | 7073 | GGCAACCAUUUCUUGAAGA |
| siRNA 720 | 720 | CUUCAAGAAAUGGUUGCCA | 7074 | UGGCAACCAUUUCUUGAAG |
| siRNA 721 | 721 | UUCAAGAAAUGGUUGCCAC | 7075 | GUGGCAACCAUUUCUUGAA |
| siRNA 722 | 722 | UCAAGAAAUGGUUGCCACU | 7076 | AGUGGCAACCAUUUCUUGA |
| siRNA 723 | 723 | CAAGAAAUGGUUGCCACUG | 7077 | CAGUGGCAACCAUUUCUUG |
| siRNA 724 | 724 | AAGAAAUGGUUGCCACUG | 7078 | ACAGUGGCAACCAUUUCUU |
| | | U | | |
| siRNA 725 | 725 | AGAAAUGGUUGCCACUGUC | 7079 | GACAGUGGCAACCAUUUCU |
| siRNA 726 | 726 | GAAAUGGUUGCCACUGUCU | 7080 | AGACAGUGGCAACCAUUUC |
| siRNA 727 | 727 | AAAUGGUUGCCACUGUCUC | 7081 | GAGACAGUGGCAACCAUUU |
| siRNA 728 | 728 | AAUGGUUGCCACUGUCUCA | 7082 | UGAGACAGUGGCAACCAUU |
| siRNA 729 | 729 | AUGGUUGCCACUGUCUCAC | 7083 | GUGAGACAGUGGCAACCAU |
| siRNA 730 | 730 | UGGUUGCCACUGUCUCACC | 7084 | GGUGAGACAGUGGCAACCA |
| siRNA 731 | 731 | GGUUGCCACUGUCUCACCG | 7085 | CGGUGAGACAGUGGCAACC |
| siRNA 732 | 732 | GUUGCCACUGUCUCACCGG | 7086 | CCGGUGAGACAGUGGCAAC |
| siRNA 733 | 733 | UUGCCACUGUCUCACCGGC | 7087 | GCCGGUGAGACAGUGGCAA |
| siRNA 734 | 734 | UGCCACUGUCUCACCGGCA | 7088 | UGCCGGUGAGACAGUGGCA |
| siRNA 735 | 735 | GCCACUGUCUCACCGGCAA | 7089 | UUGCCGGUGAGACAGUGGC |
| siRNA 736 | 736 | CCACUGUCUCACCGGCAAU | 7090 | AUUGCCGGUGAGACAGUG |
| | | | | G |
| siRNA 737 | 737 | CACUGUCUCACCGGCAAUG | 7091 | CAUUGCCGGUGAGACAGUG |
| siRNA 738 | 738 | ACUGUCUCACCGGCAAUGA | 7092 | UCAUUGCCGGUGAGACAGU |
| siRNA 739 | 739 | CUGUCUCACCGGCAAUGAU | 7093 | AUCAUUGCCGGUGAGACAG |
| siRNA 740 | 740 | UGUCUCACCGGCAAUGAUC | 7094 | GAUCAUUGCCGGUGAGACA |
| siRNA 741 | 741 | GUCUCACCGGCAAUGAUCA | 7095 | UGAUCAUUGCCGGUGAGAC |
| siRNA 742 | 742 | UCUCACCGGCAAUGAUCAG | 7096 | CUGAUCAUUGCCGGUGAGA |
| siRNA 743 | 743 | CUCACCGGCAAUGAUCAGA | 7097 | UCUGAUCAUUGCCGGUGAG |
| siRNA 744 | 744 | UCACCGGCAAUGAUCAGAC | 7098 | GUCUGAUCAUUGCCGGUGA |
| siRNA 745 | 745 | CACCGGCAAUGAUCAGACU | 7099 | AGUCUGAUCAUUGCCGGUG |
| siRNA 746 | 746 | ACCGGCAAUGAUCAGACUG | 7100 | CAGUCUGAUCAUUGCCGGU |
| siRNA 747 | 747 | CCGGCAAUGAUCAGACUGA | 7101 | UCAGUCUGAUCAUUGCCGG |
| siRNA 748 | 748 | CGGCAAUGAUCAGACUGAC | 7102 | GUCAGUCUGAUCAUUGCCG |
| siRNA 749 | 749 | GGCAAUGAUCAGACUGACU | 7103 | AGUCAGUCUGAUCAUUGCC |
| siRNA 750 | 750 | GCAAUGAUCAGACUGACUG | 7104 | CAGUCAGUCUGAUCAUUGC |
| siRNA 751 | 751 | CAAUGAUCAGACUGACUGG | 7105 | CCAGUCAGUCUGAUCAUUG |
| siRNA 752 | 752 | AAUGAUCAGACUGACUGG | 7106 | CCCAGUCAGUCUGAUCAUU |
| | | G | | |
| siRNA 753 | 753 | AUGAUCAGACUGACUGGG | 7107 | ACCCAGUCAGUCUGAUCAU |
| | | U | | |
| siRNA 754 | 754 | UGAUCAGACUGACUGGGU | 7108 | CACCCAGUCAGUCUGAUCA |
| | | G | | |
| siRNA 755 | 755 | GAUCAGACUGACUGGGUG | 7109 | CCACCCAGUCAGUCUGAUC |
| | | G | | |
| siRNA 756 | 756 | AUCAGACUGACUGGGUGG | 7110 | CCCACCCAGUCAGUCUGAU |
| | | G | | |
| siRNA 757 | 757 | UCAGACUGACUGGGUGGG | 7111 | ACCCACCCAGUCAGUCUGA |
| | | U | | |
| siRNA 758 | 758 | CAGACUGACUGGGUGGGU | 7112 | CACCCACCCAGUCAGUCUG |
| | | G | | |
| siRNA 759 | 759 | AGACUGACUGGGUGGGUG | 7113 | GCACCCACCCAGUCAGUCU |
| | | C | | |
| siRNA 760 | 760 | GACUGACUGGGUGGGUGC | 7114 | AGCACCCACCCAGUCAGUC |
| | | U | | |
| siRNA 761 | 761 | ACUGACUGGGUGGGUGCU | 7115 | CAGCACCCACCCAGUCAGU |
| | | G | | |
| siRNA 762 | 762 | CUGACUGGGUGGGUGCUGC | 7116 | GCAGCACCCACCCAGUCAG |
| siRNA 763 | 763 | UGACUGGGUGGGUGCUGC | 7117 | AGCAGCACCCACCCAGUCA |
| | | U | | |
| siRNA 764 | 764 | GACUGGGUGGGUGCUGCU | 7118 | UAGCAGCACCCACCCAGUC |
| | | A | | |
| siRNA 765 | 765 | ACUGGGUGGGUGCUGCUA | 7119 | UUAGCAGCACCCACCCAGU |
| | | A | | |
| siRNA 766 | 766 | CUGGGUGGGUGCUGCUAA | 7120 | UUUAGCAGCACCCACCCAG |
| | | A | | |
| siRNA 767 | 767 | UGGGUGGGUGCUGCUAAA | 7121 | UUUUAGCAGCACCCACCCA |
| | | A | | |
| siRNA 768 | 768 | GGGUGGGUGCUGCUAAAA | 7122 | GUUUUAGCAGCACCCACCC |
| | | C | | |
| siRNA 769 | 769 | GGUGGGUGCUGCUAAAAC | 7123 | AGUUUUAGCAGCACCCACC |
| | | U | | |
| siRNA 770 | 770 | GUGGGUGCUGCUAAAACU | 7124 | CAGUUUUAGCAGCACCCAC |
| | | G | | |
| siRNA 771 | 771 | UGGGUGCUGCUAAAACUG | 7125 | ACAGUUUUAGCAGCACCCA |
| | | U | | |
| siRNA 772 | 772 | GGGUGCUGCUAAAACUGU | 7126 | AACAGUUUUAGCAGCACCC |
| | | U | | |
| siRNA 773 | 773 | GGUGCUGCUAAAACUGUUC | 7127 | GAACAGUUUUAGCAGCACC |
| siRNA 774 | 774 | GUGCUGCUAAAACUGUUCA | 7128 | UGAACAGUUUUAGCAGCAC |
| siRNA 775 | 775 | UGCUGCUAAAACUGUUCAA | 7129 | UUGAACAGUUUUAGCAGC |
| | | | | A |
| siRNA 776 | 776 | GCUGCUAAAACUGUUCAAC | 7130 | GUUGAACAGUUUUAGCAG |
| | | | | C |
| siRNA 777 | 777 | CUGCUAAAACUGUUCAACA | 7131 | UGUUGAACAGUUUUAGCA |
| | | | | G |
| siRNA 778 | 778 | UGCUAAAACUGUUCAACAG | 7132 | CUGUUGAACAGUUUUAGC |
| | | | | A |
| siRNA 779 | 779 | GCUAAAACUGUUCAACAGC | 7133 | GCUGUUGAACAGUUUUAG |
| | | | | C |
| siRNA 780 | 780 | CUAAAACUGUUCAACAGCU | 7134 | AGCUGUUGAACAGUUUUA |
| | | | | G |
| siRNA 781 | 781 | UAAAACUGUUCAACAGCUU | 7135 | AAGCUGUUGAACAGUUUU |
| | | | | A |
| siRNA 782 | 782 | AAAACUGUUCAACAGCUUC | 7136 | GAAGCUGUUGAACAGUUU |
| | | | | U |
| siRNA 783 | 783 | AAACUGUUCAACAGCUUCU | 7137 | AGAAGCUGUUGAACAGUU |
| | | | | U |
| siRNA 784 | 784 | AACUGUUCAACAGCUUCUU | 7138 | AAGAAGCUGUUGAACAGU |
| | | | | U |
| siRNA 785 | 785 | ACUGUUCAACAGCUUCUUU | 7139 | AAAGAAGCUGUUGAACAG |
| | | | | U |
| siRNA 786 | 786 | CUGUUCAACAGCUUCUUUU | 7140 | AAAAGAAGCUGUUGAACA |
| | | | | G |
| siRNA 787 | 787 | UGUUCAACAGCUUCUUUUG | 7141 | CAAAAGAAGCUGUUGAAC |
| | | | | A |
| siRNA 788 | 788 | GUUCAACAGCUUCUUUUGG | 7142 | CCAAAAGAAGCUGUUGAAC |
| siRNA 789 | 789 | UUCAACAGCUUCUUUUGGA | 7143 | UCCAAAAGAAGCUGUUGA |
| | | | | A |
| siRNA 790 | 790 | UCAACAGCUUCUUUUGGAA | 7144 | UUCCAAAAGAAGCUGUUG |
| | | | | A |
| siRNA 791 | 791 | CAACAGCUUCUUUUGGAAC | 7145 | GUUCCAAAAGAAGCUGUU |
| | | | | G |
| siRNA 792 | 792 | AACAGCUUCUUUUGGAACA | 7146 | UGUUCCAAAAGAAGCUGU |
| | | | | U |
| siRNA 793 | 793 | ACAGCUUCUUUUGGAACAU | 7147 | AUGUUCCAAAAGAAGCUG |
| | | | | U |
| siRNA 794 | 794 | CAGCUUCUUUUGGAACAUU | 7148 | AAUGUUCCAAAAGAAGCU |
| | | | | G |
| siRNA 795 | 795 | AGCUUCUUUUGGAACAUUC | 7149 | GAAUGUUCCAAAAGAAGC |
| | | | | U |
| siRNA 796 | 796 | GCUUCUUUUGGAACAUUCA | 7150 | UGAAUGUUCCAAAAGAAG |
| | | | | C |
| siRNA 797 | 797 | CUUCUUUUGGAACAUUCAA | 7151 | UUGAAUGUUCCAAAAGAA |
| | | | | G |
| siRNA 798 | 798 | UUCUUUUGGAACAUUCAA | 7152 | UUUGAAUGUUCCAAAAGA |
| | | A | | A |
| siRNA 799 | 799 | UCUUUUGGAACAUUCAAA | 7153 | AUUUGAAUGUUCCAAAAG |
| | | U | | A |
| siRNA 800 | 800 | CUUUUGGAACAUUCAAAU | 7154 | AAUUUGAAUGUUCCAAAA |
| | | U | | G |
| siRNA 801 | 801 | UUUUGGAACAUUCAAAUU | 7155 | GAAUUUGAAUGUUCCAAA |
| | | C | | A |
| siRNA 802 | 802 | UUUGGAACAUUCAAAUUC | 7156 | UGAAUUUGAAUGUUCCAA |
| | | A | | A |
| siRNA 803 | 803 | UUGGAACAUUCAAAUUCAC | 7157 | GUGAAUUUGAAUGUUCCA |
| | | | | A |
| siRNA 804 | 804 | UGGAACAUUCAAAUUCACA | 7158 | UGUGAAUUUGAAUGUUCC |
| | | | | A |
| siRNA 805 | 805 | GGAACAUUCAAAUUCACAA | 7159 | UUGUGAAUUUGAAUGUUC |
| | | | | C |
| siRNA 806 | 806 | GAACAUUCAAAUUCACAAA | 7160 | UUUGUGAAUUUGAAUGUU |
| | | | | C |
| siRNA 807 | 807 | AACAUUCAAAUUCACAAAG | 7161 | CUUUGUGAAUUUGAAUGU |
| | | | | U |
| siRNA 808 | 808 | ACAUUCAAAUUCACAAAGG | 7162 | CCUUUGUGAAUUUGAAUG |
| | | | | U |
| siRNA 809 | 809 | CAUUCAAAUUCACAAAGGU | 7163 | ACCUUUGUGAAUUUGAAU |
| | | | | G |
| siRNA 810 | 810 | AUUCAAAUUCACAAAGGUC | 7164 | GACCUUUGUGAAUUUGAA |
| | | | | U |
| siRNA 811 | 811 | UUCAAAUUCACAAAGGUCA | 7165 | UGACCUUUGUGAAUUUGA |
| | | | | A |
| siRNA 812 | 812 | UCAAAUUCACAAAGGUCAA | 7166 | UUGACCUUUGUGAAUUUG |
| | | | | A |
| siRNA 813 | 813 | CAAAUUCACAAAGGUCAAC | 7167 | GUUGACCUUUGUGAAUUU |
| | | | | G |
| siRNA 814 | 814 | AAAUUCACAAAGGUCAACU | 7168 | AGUUGACCUUUGUGAAUU |
| | | | | U |
| siRNA 815 | 815 | AAUUCACAAAGGUCAACUU | 7169 | AAGUUGACCUUUGUGAAU |
| | | | | U |
| siRNA 816 | 816 | AUUCACAAAGGUCAACUUG | 7170 | CAAGUUGACCUUUGUGAA |
| | | | | U |
| siRNA 817 | 817 | UUCACAAAGGUCAACUUGA | 7171 | UCAAGUUGACCUUUGUGA |
| | | | | A |
| siRNA 818 | 818 | UCACAAAGGUCAACUUGAG | 7172 | CUCAAGUUGACCUUUGUGA |
| siRNA 819 | 819 | CACAAAGGUCAACUUGAGA | 7173 | UCUCAAGUUGACCUUUGUG |
| siRNA 820 | 820 | ACAAAGGUCAACUUGAGA | 7174 | AUCUCAAGUUGACCUUUGU |
| | | U | | |
| siRNA 821 | 821 | CAAAGGUCAACUUGAGAU | 7175 | CAUCUCAAGUUGACCUUUG |
| | | G | | |
| siRNA 822 | 822 | AAAGGUCAACUUGAGAUG | 7176 | CCAUCUCAAGUUGACCUUU |
| | | G | | |
| siRNA 823 | 823 | AAGGUCAACUUGAGAUGG | 7177 | ACCAUCUCAAGUUGACCUU |
| | | U | | |
| siRNA 824 | 824 | AGGUCAACUUGAGAUGGU | 7178 | AACCAUCUCAAGUUGACCU |
| | | U | | |
| siRNA 825 | 825 | GGUCAACUUGAGAUGGUU | 7179 | UAACCAUCUCAAGUUGACC |
| | | A | | |
| siRNA 826 | 826 | GUCAACUUGAGAUGGUUA | 7180 | UUAACCAUCUCAAGUUGAC |
| | | A | | |
| siRNA 827 | 827 | UCAACUUGAGAUGGUUAA | 7181 | UUUAACCAUCUCAAGUUGA |
| | | A | | |
| siRNA 828 | 828 | CAACUUGAGAUGGUUAAA | 7182 | CUUUAACCAUCUCAAGUUG |
| | | G | | |
| siRNA 829 | 829 | AACUUGAGAUGGUUAAAG | 7183 | GCUUUAACCAUCUCAAGUU |
| | | C | | |
| siRNA 830 | 830 | ACUUGAGAUGGUUAAAGC | 7184 | AGCUUUAACCAUCUCAAGU |
| | | U | | |
| siRNA 831 | 831 | CUUGAGAUGGUUAAAGCU | 7185 | CAGCUUUAACCAUCUCAAG |
| | | G | | |
| siRNA 832 | 832 | UUGAGAUGGUUAAAGCUG | 7186 | GCAGCUUUAACCAUCUCAA |
| | | C | | |
| siRNA 833 | 833 | UGAGAUGGUUAAAGCUGC | 7187 | UGCAGCUUUAACCAUCUCA |
| | | A | | |
| siRNA 834 | 834 | GAGAUGGUUAAAGCUGCA | 7188 | UUGCAGCUUUAACCAUCUC |
| | | A | | |
| siRNA 835 | 835 | AGAUGGUUAAAGCUGCAA | 7189 | GUUGCAGCUUUAACCAUCU |
| | | C | | |
| siRNA 836 | 836 | GAUGGUUAAAGCUGCAAC | 7190 | AGUUGCAGCUUUAACCAUC |
| | | U | | |
| siRNA 837 | 837 | AUGGUUAAAGCUGCAACU | 7191 | CAGUUGCAGCUUUAACCAU |
| | | G | | |
| siRNA 838 | 838 | UGGUUAAAGCUGCAACUG | 7192 | UCAGUUGCAGCUUUAACCA |
| | | A | | |
| siRNA 839 | 839 | GGUUAAAGCUGCAACUGA | 7193 | CUCAGUUGCAGCUUUAACC |
| | | G | | |
| siRNA 840 | 840 | GUUAAAGCUGCAACUGAG | 7194 | UCUCAGUUGCAGCUUUAAC |
| | | A | | |
| siRNA 841 | 841 | UUAAAGCUGCAACUGAGAC | 7195 | GUCUCAGUUGCAGCUUUAA |
| siRNA 842 | 842 | UAAAGCUGCAACUGAGACG | 7196 | CGUCUCAGUUGCAGCUUUA |
| siRNA 843 | 843 | AAAGCUGCAACUGAGACGA | 7197 | UCGUCUCAGUUGCAGCUUU |
| siRNA 844 | 844 | AAGCUGCAACUGAGACGAA | 7198 | UUCGUCUCAGUUGCAGCUU |
| siRNA 845 | 845 | AGCUGCAACUGAGACGAAU | 7199 | AUUCGUCUCAGUUGCAGCU |
| siRNA 846 | 846 | GCUGCAACUGAGACGAAUU | 7200 | AAUUCGUCUCAGUUGCAGC |
| siRNA 847 | 847 | CUGCAACUGAGACGAAUUU | 7201 | AAAUUCGUCUCAGUUGCAG |
| siRNA 848 | 848 | UGCAACUGAGACGAAUUU | 7202 | CAAAUUCGUCUCAGUUGCA |
| | | G | | |
| siRNA 849 | 849 | GCAACUGAGACGAAUUUGC | 7203 | GCAAAUUCGUCUCAGUUGC |
| siRNA 850 | 850 | CAACUGAGACGAAUUUGCC | 7204 | GGCAAAUUCGUCUCAGUUG |
| siRNA 851 | 851 | AACUGAGACGAAUUUGCCG | 7205 | CGGCAAAUUCGUCUCAGUU |
| siRNA 852 | 852 | ACUGAGACGAAUUUGCCGC | 7206 | GCGGCAAAUUCGUCUCAGU |
| siRNA 853 | 853 | CUGAGACGAAUUUGCCGCU | 7207 | AGCGGCAAAUUCGUCUCAG |
| siRNA 854 | 854 | UGAGACGAAUUUGCCGCUU | 7208 | AAGCGGCAAAUUCGUCUCA |
| siRNA 855 | 855 | GAGACGAAUUUGCCGCUUC | 7209 | GAAGCGGCAAAUUCGUCUC |
| siRNA 856 | 856 | AGACGAAUUUGCCGCUUCU | 7210 | AGAAGCGGCAAAUUCGUCU |
| siRNA 857 | 857 | GACGAAUUUGCCGCUUCUG | 7211 | CAGAAGCGGCAAAUUCGUC |
| siRNA 858 | 858 | ACGAAUUUGCCGCUUCUGU | 7212 | ACAGAAGCGGCAAAUUCGU |
| siRNA 859 | 859 | CGAAUUUGCCGCUUCUGUU | 7213 | AACAGAAGCGGCAAAUUCG |
| siRNA 860 | 860 | GAAUUUGCCGCUUCUGUUU | 7214 | AAACAGAAGCGGCAAAUUC |
| siRNA 861 | 861 | AAUUUGCCGCUUCUGUUUC | 7215 | GAAACAGAAGCGGCAAAU |
| | | | | U |
| siRNA 862 | 862 | AUUUGCCGCUUCUGUUUCU | 7216 | AGAAACAGAAGCGGCAAA |
| | | | | U |
| siRNA 863 | 863 | UUUGCCGCUUCUGUUUCUA | 7217 | UAGAAACAGAAGCGGCAA |
| | | | | A |
| siRNA 864 | 864 | UUGCCGCUUCUGUUUCUAC | 7218 | GUAGAAACAGAAGCGGCA |
| | | | | A |
| siRNA 865 | 865 | UGCCGCUUCUGUUUCUACC | 7219 | GGUAGAAACAGAAGCGGC |
| | | | | A |
| siRNA 866 | 866 | GCCGCUUCUGUUUCUACCA | 7220 | UGGUAGAAACAGAAGCGG |
| | | | | C |
| siRNA 867 | 867 | CCGCUUCUGUUUCUACCAG | 7221 | CUGGUAGAAACAGAAGCG |
| | | | | G |
| siRNA 868 | 868 | CGCUUCUGUUUCUACCAGU | 7222 | ACUGGUAGAAACAGAAGC |
| | | | | G |
| siRNA 869 | 869 | GCUUCUGUUUCUACCAGUU | 7223 | AACUGGUAGAAACAGAAG |
| | | | | C |
| siRNA 870 | 870 | CUUCUGUUUCUACCAGUUC | 7224 | GAACUGGUAGAAACAGAA |
| | | | | G |
| siRNA 871 | 871 | UUCUGUUUCUACCAGUUCA | 7225 | UGAACUGGUAGAAACAGA |
| | | | | A |
| siRNA 872 | 872 | UCUGUUUCUACCAGUUCAU | 7226 | AUGAACUGGUAGAAACAG |
| | | | | A |
| siRNA 873 | 873 | CUGUUUCUACCAGUUCAUA | 7227 | UAUGAACUGGUAGAAACA |
| | | | | G |
| siRNA 874 | 874 | UGUUUCUACCAGUUCAUAG | 7228 | CUAUGAACUGGUAGAAAC |
| | | | | A |
| siRNA 875 | 875 | GUUUCUACCAGUUCAUAGA | 7229 | UCUAUGAACUGGUAGAAA |
| | | | | C |
| siRNA 876 | 876 | UUUCUACCAGUUCAUAGAU | 7230 | AUCUAUGAACUGGUAGAA |
| | | | | A |
| siRNA 877 | 877 | UUCUACCAGUUCAUAGAUC | 7231 | GAUCUAUGAACUGGUAGA |
| | | | | A |
| siRNA 878 | 878 | UCUACCAGUUCAUAGAUCC | 7232 | GGAUCUAUGAACUGGUAG |
| | | | | A |
| siRNA 879 | 879 | CUACCAGUUCAUAGAUCCC | 7233 | GGGAUCUAUGAACUGGUA |
| | | | | G |
| siRNA 880 | 880 | UACCAGUUCAUAGAUCCCA | 7234 | UGGGAUCUAUGAACUGGU |
| | | | | A |
| siRNA 881 | 881 | ACCAGUUCAUAGAUCCCAU | 7235 | AUGGGAUCUAUGAACUGG |
| | | | | U |
| siRNA 882 | 882 | CCAGUUCAUAGAUCCCAUA | 7236 | UAUGGGAUCUAUGAACUG |
| | | | | G |
| siRNA 883 | 883 | CAGUUCAUAGAUCCCAUAU | 7237 | AUAUGGGAUCUAUGAACU |
| | | | | G |
| siRNA 884 | 884 | AGUUCAUAGAUCCCAUAUU | 7238 | AAUAUGGGAUCUAUGAAC |
| | | | | U |
| siRNA 885 | 885 | GUUCAUAGAUCCCAUAUUG | 7239 | CAAUAUGGGAUCUAUGAA |
| | | | | C |
| siRNA 886 | 886 | UUCAUAGAUCCCAUAUUGA | 7240 | UCAAUAUGGGAUCUAUGA |
| | | | | A |
| siRNA 887 | 887 | UCAUAGAUCCCAUAUUGAC | 7241 | GUCAAUAUGGGAUCUAUG |
| | | | | A |
| siRNA 888 | 888 | CAUAGAUCCCAUAUUGACU | 7242 | AGUCAAUAUGGGAUCUAU |
| | | | | G |
| siRNA 889 | 889 | AUAGAUCCCAUAUUGACUA | 7243 | UAGUCAAUAUGGGAUCUA |
| | | | | U |
| siRNA 890 | 890 | UAGAUCCCAUAUUGACUAU | 7244 | AUAGUCAAUAUGGGAUCU |
| | | | | A |
| siRNA 891 | 891 | AGAUCCCAUAUUGACUAUC | 7245 | GAUAGUCAAUAUGGGAUC |
| | | | | U |
| siRNA 892 | 892 | GAUCCCAUAUUGACUAUCU | 7246 | AGAUAGUCAAUAUGGGAU |
| | | | | C |
| siRNA 893 | 893 | AUCCCAUAUUGACUAUCUG | 7247 | CAGAUAGUCAAUAUGGGA |
| | | | | U |
| siRNA 894 | 894 | UCCCAUAUUGACUAUCUGC | 7248 | GCAGAUAGUCAAUAUGGG |
| | | | | A |
| siRNA 895 | 895 | CCCAUAUUGACUAUCUGCU | 7249 | AGCAGAUAGUCAAUAUGG |
| | | | | G |
| siRNA 896 | 896 | CCAUAUUGACUAUCUGCUG | 7250 | CAGCAGAUAGUCAAUAUG |
| | | | | G |
| siRNA 897 | 897 | CAUAUUGACUAUCUGCUGC | 7251 | GCAGCAGAUAGUCAAUAU |
| | | | | G |
| siRNA 898 | 898 | AUAUUGACUAUCUGCUGCU | 7252 | AGCAGCAGAUAGUCAAUA |
| | | | | U |
| siRNA 899 | 899 | UAUUGACUAUCUGCUGCUC | 7253 | GAGCAGCAGAUAGUCAAU |
| | | | | A |
| siRNA 900 | 900 | AUUGACUAUCUGCUGCUCA | 7254 | UGAGCAGCAGAUAGUCAA |
| | | | | U |
| siRNA 901 | 901 | UUGACUAUCUGCUGCUCAC | 7255 | GUGAGCAGCAGAUAGUCA |
| | | | | A |
| siRNA 902 | 902 | UGACUAUCUGCUGCUCACU | 7256 | AGUGAGCAGCAGAUAGUC |
| | | | | A |
| siRNA 903 | 903 | GACUAUCUGCUGCUCACUU | 7257 | AAGUGAGCAGCAGAUAGU |
| | | | | C |
| siRNA 904 | 904 | ACUAUCUGCUGCUCACUUU | 7258 | AAAGUGAGCAGCAGAUAG |
| | | | | U |
| siRNA 905 | 905 | CUAUCUGCUGCUCACUUUC | 7259 | GAAAGUGAGCAGCAGAUA |
| | | | | G |
| siRNA 906 | 906 | UAUCUGCUGCUCACUUUCA | 7260 | UGAAAGUGAGCAGCAGAU |
| | | | | A |
| siRNA 907 | 907 | AUCUGCUGCUCACUUUCAU | 7261 | AUGAAAGUGAGCAGCAGA |
| | | | | U |
| siRNA 908 | 908 | UCUGCUGCUCACUUUCAUU | 7262 | AAUGAAAGUGAGCAGCAG |
| | | | | A |
| siRNA 909 | 909 | CUGCUGCUCACUUUCAUUC | 7263 | GAAUGAAAGUGAGCAGCA |
| | | | | G |
| siRNA 910 | 910 | UGCUGCUCACUUUCAUUCU | 7264 | AGAAUGAAAGUGAGCAGC |
| | | | | A |
| siRNA 911 | 911 | GCUGCUCACUUUCAUUCUC | 7265 | GAGAAUGAAAGUGAGCAG |
| | | | | C |
| siRNA 912 | 912 | CUGCUCACUUUCAUUCUCU | 7266 | AGAGAAUGAAAGUGAGCA |
| | | | | G |
| siRNA 913 | 913 | UGCUCACUUUCAUUCUCUU | 7267 | AAGAGAAUGAAAGUGAGC |
| | | | | A |
| siRNA 914 | 914 | GCUCACUUUCAUUCUCUUC | 7268 | GAAGAGAAUGAAAGUGAG |
| | | | | C |
| siRNA 915 | 915 | CUCACUUUCAUUCUCUUCU | 7269 | AGAAGAGAAUGAAAGUGA |
| | | | | G |
| siRNA 916 | 916 | UCACUUUCAUUCUCUUCUG | 7270 | CAGAAGAGAAUGAAAGUG |
| | | | | A |
| siRNA 917 | 917 | CACUUUCAUUCUCUUCUGC | 7271 | GCAGAAGAGAAUGAAAGU |
| | | | | G |
| siRNA 918 | 918 | ACUUUCAUUCUCUUCUGCC | 7272 | GGCAGAAGAGAAUGAAAG |
| | | | | U |
| siRNA 919 | 919 | CUUUCAUUCUCUUCUGCCA | 7273 | UGGCAGAAGAGAAUGAAA |
| | | | | G |
| siRNA 920 | 920 | UUUCAUUCUCUUCUGCCAU | 7274 | AUGGCAGAAGAGAAUGAA |
| | | | | A |
| siRNA 921 | 921 | UUCAUUCUCUUCUGCCAUA | 7275 | UAUGGCAGAAGAGAAUGA |
| | | | | A |
| siRNA 922 | 922 | UCAUUCUCUUCUGCCAUAA | 7276 | UUAUGGCAGAAGAGAAUG |
| | | | | A |
| siRNA 923 | 923 | CAUUCUCUUCUGCCAUAAC | 7277 | GUUAUGGCAGAAGAGAAU |
| | | | | G |
| siRNA 924 | 924 | AUUCUCUUCUGCCAUAACA | 7278 | UGUUAUGGCAGAAGAGAA |
| | | | | U |
| siRNA 925 | 925 | UUCUCUUCUGCCAUAACAU | 7279 | AUGUUAUGGCAGAAGAGA |
| | | | | A |
| siRNA 926 | 926 | UCUCUUCUGCCAUAACAUC | 7280 | GAUGUUAUGGCAGAAGAG |
| | | | | A |
| siRNA 927 | 927 | CUCUUCUGCCAUAACAUCA | 7281 | UGAUGUUAUGGCAGAAGA |
| | | | | G |
| siRNA 928 | 928 | UCUUCUGCCAUAACAUCAA | 7282 | UUGAUGUUAUGGCAGAAG |
| | | | | A |
| siRNA 929 | 929 | CUUCUGCCAUAACAUCAAA | 7283 | UUUGAUGUUAUGGCAGAA |
| | | | | G |
| siRNA 930 | 930 | UUCUGCCAUAACAUCAAAG | 7284 | CUUUGAUGUUAUGGCAGA |
| | | | | A |
| siRNA 931 | 931 | UCUGCCAUAACAUCAAAGC | 7285 | GCUUUGAUGUUAUGGCAG |
| | | | | A |
| siRNA 932 | 932 | CUGCCAUAACAUCAAAGCA | 7286 | UGCUUUGAUGUUAUGGCA |
| | | | | G |
| siRNA 933 | 933 | UGCCAUAACAUCAAAGCAC | 7287 | GUGCUUUGAUGUUAUGGC |
| | | | | A |
| siRNA 934 | 934 | GCCAUAACAUCAAAGCACC | 7288 | GGUGCUUUGAUGUUAUGG |
| | | | | C |
| siRNA 935 | 935 | CCAUAACAUCAAAGCACCA | 7289 | UGGUGCUUUGAUGUUAUG |
| | | | | G |
| siRNA 936 | 936 | CAUAACAUCAAAGCACCAU | 7290 | AUGGUGCUUUGAUGUUAU |
| | | | | G |
| siRNA 937 | 937 | AUAACAUCAAAGCACCAUA | 7291 | UAUGGUGCUUUGAUGUUA |
| | | | | U |
| siRNA 938 | 938 | UAACAUCAAAGCACCAUAC | 7292 | GUAUGGUGCUUUGAUGUU |
| | | | | A |
| siRNA 939 | 939 | AACAUCAAAGCACCAUACA | 7293 | UGUAUGGUGCUUUGAUGU |
| | | | | U |
| siRNA 940 | 940 | ACAUCAAAGCACCAUACAU | 7294 | AUGUAUGGUGCUUUGAUG |
| | | | | U |
| siRNA 941 | 941 | CAUCAAAGCACCAUACAUU | 7295 | AAUGUAUGGUGCUUUGAU |
| | | | | G |
| siRNA 942 | 942 | AUCAAAGCACCAUACAUUG | 7296 | CAAUGUAUGGUGCUUUGA |
| | | | | U |
| siRNA 943 | 943 | UCAAAGCACCAUACAUUGC | 7297 | GCAAUGUAUGGUGCUUUG |
| | | | | A |
| siRNA 944 | 944 | CAAAGCACCAUACAUUGCU | 7298 | AGCAAUGUAUGGUGCUUU |
| | | | | G |
| siRNA 945 | 945 | AAAGCACCAUACAUUGCUU | 7299 | AAGCAAUGUAUGGUGCUU |
| | | | | U |
| siRNA 946 | 946 | AAGCACCAUACAUUGCUUC | 7300 | GAAGCAAUGUAUGGUGCU |
| | | | | U |
| siRNA 947 | 947 | AGCACCAUACAUUGCUUCA | 7301 | UGAAGCAAUGUAUGGUGC |
| | | | | U |
| siRNA 948 | 948 | GCACCAUACAUUGCUUCAG | 7302 | CUGAAGCAAUGUAUGGUG |
| | | | | C |
| siRNA 949 | 949 | CACCAUACAUUGCUUCAGG | 7303 | CCUGAAGCAAUGUAUGGU |
| | | | | G |
| siRNA 950 | 950 | ACCAUACAUUGCUUCAGGC | 7304 | GCCUGAAGCAAUGUAUGG |
| | | | | U |
| siRNA 951 | 951 | CCAUACAUUGCUUCAGGCA | 7305 | UGCCUGAAGCAAUGUAUG |
| | | | | G |
| siRNA 952 | 952 | CAUACAUUGCUUCAGGCAA | 7306 | UUGCCUGAAGCAAUGUAU |
| | | | | G |
| siRNA 953 | 953 | AUACAUUGCUUCAGGCAAU | 7307 | AUUGCCUGAAGCAAUGUA |
| | | | | U |
| siRNA 954 | 954 | UACAUUGCUUCAGGCAAUA | 7308 | UAUUGCCUGAAGCAAUGU |
| | | | | A |
| siRNA 955 | 955 | ACAUUGCUUCAGGCAAUAA | 7309 | UUAUUGCCUGAAGCAAUG |
| | | | | U |
| siRNA 956 | 956 | CAUUGCUUCAGGCAAUAAU | 7310 | AUUAUUGCCUGAAGCAAU |
| | | | | G |
| siRNA 957 | 957 | AUUGCUUCAGGCAAUAAUC | 7311 | GAUUAUUGCCUGAAGCAA |
| | | | | U |
| siRNA 958 | 958 | UUGCUUCAGGCAAUAAUCU | 7312 | AGAUUAUUGCCUGAAGCA |
| | | | | A |
| siRNA 959 | 959 | UGCUUCAGGCAAUAAUCUC | 7313 | GAGAUUAUUGCCUGAAGC |
| | | | | A |
| siRNA 960 | 960 | GCUUCAGGCAAUAAUCUCA | 7314 | UGAGAUUAUUGCCUGAAG |
| | | | | C |
| siRNA 961 | 961 | CUUCAGGCAAUAAUCUCAA | 7315 | UUGAGAUUAUUGCCUGAA |
| | | | | G |
| siRNA 962 | 962 | UUCAGGCAAUAAUCUCAAC | 7316 | GUUGAGAUUAUUGCCUGA |
| | | | | A |
| siRNA 963 | 963 | UCAGGCAAUAAUCUCAACA | 7317 | UGUUGAGAUUAUUGCCUG |
| | | | | A |
| siRNA 964 | 964 | CAGGCAAUAAUCUCAACAU | 7318 | AUGUUGAGAUUAUUGCCU |
| | | | | G |
| siRNA 965 | 965 | AGGCAAUAAUCUCAACAUC | 7319 | GAUGUUGAGAUUAUUGCC |
| | | | | U |
| siRNA 966 | 966 | GGCAAUAAUCUCAACAUCC | 7320 | GGAUGUUGAGAUUAUUGC |
| | | | | C |
| siRNA 967 | 967 | GCAAUAAUCUCAACAUCCC | 7321 | GGGAUGUUGAGAUUAUUG |
| | | | | C |
| siRNA 968 | 968 | CAAUAAUCUCAACAUCCCA | 7322 | UGGGAUGUUGAGAUUAUU |
| | | | | G |
| siRNA 969 | 969 | AAUAAUCUCAACAUCCCAA | 7323 | UUGGGAUGUUGAGAUUAU |
| | | | | U |
| siRNA 970 | 970 | AUAAUCUCAACAUCCCAAU | 7324 | AUUGGGAUGUUGAGAUUA |
| | | | | U |
| siRNA 971 | 971 | UAAUCUCAACAUCCCAAUC | 7325 | GAUUGGGAUGUUGAGAUU |
| | | | | A |
| siRNA 972 | 972 | AAUCUCAACAUCCCAAUCU | 7326 | AGAUUGGGAUGUUGAGAU |
| | | | | U |
| siRNA 973 | 973 | AUCUCAACAUCCCAAUCUU | 7327 | AAGAUUGGGAUGUUGAGA |
| | | | | U |
| siRNA 974 | 974 | UCUCAACAUCCCAAUCUUC | 7328 | GAAGAUUGGGAUGUUGAG |
| | | | | A |
| siRNA 975 | 975 | CUCAACAUCCCAAUCUUCA | 7329 | UGAAGAUUGGGAUGUUGA |
| | | | | G |
| siRNA 976 | 976 | UCAACAUCCCAAUCUUCAG | 7330 | CUGAAGAUUGGGAUGUUG |
| | | | | A |
| siRNA 977 | 977 | CAACAUCCCAAUCUUCAGU | 7331 | ACUGAAGAUUGGGAUGUU |
| | | | | G |
| siRNA 978 | 978 | AACAUCCCAAUCUUCAGUA | 7332 | UACUGAAGAUUGGGAUGU |
| | | | | U |
| siRNA 979 | 979 | ACAUCCCAAUCUUCAGUAC | 7333 | GUACUGAAGAUUGGGAUG |
| | | | | U |
| siRNA 980 | 980 | CAUCCCAAUCUUCAGUACC | 7334 | GGUACUGAAGAUUGGGAU |
| | | | | G |
| siRNA 981 | 981 | AUCCCAAUCUUCAGUACCU | 7335 | AGGUACUGAAGAUUGGGA |
| | | | | U |
| siRNA 982 | 982 | UCCCAAUCUUCAGUACCUU | 7336 | AAGGUACUGAAGAUUGGG |
| | | | | A |
| siRNA 983 | 983 | CCCAAUCUUCAGUACCUUG | 7337 | CAAGGUACUGAAGAUUGG |
| | | | | G |
| siRNA 984 | 984 | CCAAUCUUCAGUACCUUGA | 7338 | UCAAGGUACUGAAGAUUG |
| | | | | G |
| siRNA 985 | 985 | CAAUCUUCAGUACCUUGAU | 7339 | AUCAAGGUACUGAAGAUU |
| | | | | G |
| siRNA 986 | 986 | AAUCUUCAGUACCUUGAUC | 7340 | GAUCAAGGUACUGAAGAU |
| | | | | U |
| siRNA 987 | 987 | AUCUUCAGUACCUUGAUCC | 7341 | GGAUCAAGGUACUGAAGA |
| | | | | U |
| siRNA 988 | 988 | UCUUCAGUACCUUGAUCCA | 7342 | UGGAUCAAGGUACUGAAG |
| | | | | A |
| siRNA 989 | 989 | CUUCAGUACCUUGAUCCAU | 7343 | AUGGAUCAAGGUACUGAA |
| | | | | G |
| siRNA 990 | 990 | UUCAGUACCUUGAUCCAUA | 7344 | UAUGGAUCAAGGUACUGA |
| | | | | A |
| siRNA 991 | 991 | UCAGUACCUUGAUCCAUAA | 7345 | UUAUGGAUCAAGGUACUG |
| | | | | A |
| siRNA 992 | 992 | CAGUACCUUGAUCCAUAAG | 7346 | CUUAUGGAUCAAGGUACU |
| | | | | G |
| siRNA 993 | 993 | AGUACCUUGAUCCAUAAGC | 7347 | GCUUAUGGAUCAAGGUAC |
| | | | | U |
| siRNA 994 | 994 | GUACCUUGAUCCAUAAGCU | 7348 | AGCUUAUGGAUCAAGGUA |
| | | | | C |
| siRNA 995 | 995 | UACCUUGAUCCAUAAGCUU | 7349 | AAGCUUAUGGAUCAAGGU |
| | | | | A |
| siRNA 996 | 996 | ACCUUGAUCCAUAAGCUUG | 7350 | CAAGCUUAUGGAUCAAGG |
| | | | | U |
| siRNA 997 | 997 | CCUUGAUCCAUAAGCUUGG | 7351 | CCAAGCUUAUGGAUCAAGG |
| siRNA 998 | 998 | CUUGAUCCAUAAGCUUGGG | 7352 | CCCAAGCUUAUGGAUCAAG |
| siRNA 999 | 999 | UUGAUCCAUAAGCUUGGG | 7353 | CCCCAAGCUUAUGGAUCAA |
| | | G | | |
| siRNA 1000 | 1000 | UGAUCCAUAAGCUUGGGG | 7354 | CCCCCAAGCUUAUGGAUCA |
| | | G | | |
| siRNA 1001 | 1001 | GAUCCAUAAGCUUGGGGGC | 7355 | GCCCCCAAGCUUAUGGAUC |
| siRNA 1002 | 1002 | AUCCAUAAGCUUGGGGGCU | 7356 | AGCCCCCAAGCUUAUGGAU |
| siRNA 1003 | 1003 | UCCAUAAGCUUGGGGGCUU | 7357 | AAGCCCCCAAGCUUAUGGA |
| siRNA 1004 | 1004 | CCAUAAGCUUGGGGGCUUC | 7358 | GAAGCCCCCAAGCUUAUGG |
| siRNA 1005 | 1005 | CAUAAGCUUGGGGGCUUCU | 7359 | AGAAGCCCCCAAGCUUAUG |
| siRNA 1006 | 1006 | AUAAGCUUGGGGGCUUCU | 7360 | AAGAAGCCCCCAAGCUUAU |
| | | U | | |
| siRNA 1007 | 1007 | UAAGCUUGGGGGCUUCUUC | 7361 | GAAGAAGCCCCCAAGCUUA |
| siRNA 1008 | 1008 | AAGCUUGGGGGCUUCUUCA | 7362 | UGAAGAAGCCCCCAAGCUU |
| siRNA 1009 | 1009 | AGCUUGGGGGCUUCUUCAU | 7363 | AUGAAGAAGCCCCCAAGCU |
| siRNA 1010 | 1010 | GCUUGGGGGCUUCUUCAUA | 7364 | UAUGAAGAAGCCCCCAAGC |
| siRNA 1011 | 1011 | CUUGGGGGCUUCUUCAUAC | 7365 | GUAUGAAGAAGCCCCCAAG |
| siRNA 1012 | 1012 | UUGGGGGCUUCUUCAUACG | 7366 | CGUAUGAAGAAGCCCCCAA |
| siRNA 1013 | 1013 | UGGGGGCUUCUUCAUACGA | 7367 | UCGUAUGAAGAAGCCCCCA |
| siRNA 1014 | 1014 | GGGGGCUUCUUCAUACGAC | 7368 | GUCGUAUGAAGAAGCCCCC |
| siRNA 1015 | 1015 | GGGGCUUCUUCAUACGACG | 7369 | CGUCGUAUGAAGAAGCCCC |
| siRNA 1016 | 1016 | GGGCUUCUUCAUACGACGA | 7370 | UCGUCGUAUGAAGAAGCCC |
| siRNA 1017 | 1017 | GGCUUCUUCAUACGACGAA | 7371 | UUCGUCGUAUGAAGAAGCC |
| siRNA 1018 | 1018 | GCUUCUUCAUACGACGAAG | 7372 | CUUCGUCGUAUGAAGAAGC |
| siRNA 1019 | 1019 | CUUCUUCAUACGACGAAGG | 7373 | CCUUCGUCGUAUGAAGAAG |
| siRNA 1020 | 1020 | UUCUUCAUACGACGAAGGC | 7374 | GCCUUCGUCGUAUGAAGAA |
| siRNA 1021 | 1021 | UCUUCAUACGACGAAGGCU | 7375 | AGCCUUCGUCGUAUGAAGA |
| siRNA 1022 | 1022 | CUUCAUACGACGAAGGCUC | 7376 | GAGCCUUCGUCGUAUGAAG |
| siRNA 1023 | 1023 | UUCAUACGACGAAGGCUCG | 7377 | CGAGCCUUCGUCGUAUGAA |
| siRNA 1024 | 1024 | UCAUACGACGAAGGCUCGA | 7378 | UCGAGCCUUCGUCGUAUGA |
| siRNA 1025 | 1025 | CAUACGACGAAGGCUCGAU | 7379 | AUCGAGCCUUCGUCGUAUG |
| siRNA 1026 | 1026 | AUACGACGAAGGCUCGAUG | 7380 | CAUCGAGCCUUCGUCGUAU |
| siRNA 1027 | 1027 | UACGACGAAGGCUCGAUGA | 7381 | UCAUCGAGCCUUCGUCGUA |
| siRNA 1028 | 1028 | ACGACGAAGGCUCGAUGAA | 7382 | UUCAUCGAGCCUUCGUCGU |
| siRNA 1029 | 1029 | CGACGAAGGCUCGAUGAAA | 7383 | UUUCAUCGAGCCUUCGUCG |
| siRNA 1030 | 1030 | GACGAAGGCUCGAUGAAAC | 7384 | GUUUCAUCGAGCCUUCGUC |
| siRNA 1031 | 1031 | ACGAAGGCUCGAUGAAACA | 7385 | UGUUUCAUCGAGCCUUCGU |
| siRNA 1032 | 1032 | CGAAGGCUCGAUGAAACAC | 7386 | GUGUUUCAUCGAGCCUUCG |
| siRNA 1033 | 1033 | GAAGGCUCGAUGAAACACC | 7387 | GGUGUUUCAUCGAGCCUUC |
| siRNA 1034 | 1034 | AAGGCUCGAUGAAACACCA | 7388 | UGGUGUUUCAUCGAGCCUU |
| siRNA 1035 | 1035 | AGGCUCGAUGAAACACCAG | 7389 | CUGGUGUUUCAUCGAGCCU |
| siRNA 1036 | 1036 | GGCUCGAUGAAACACCAGA | 7390 | UCUGGUGUUUCAUCGAGCC |
| siRNA 1037 | 1037 | GCUCGAUGAAACACCAGAU | 7391 | AUCUGGUGUUUCAUCGAGC |
| siRNA 1038 | 1038 | CUCGAUGAAACACCAGAUG | 7392 | CAUCUGGUGUUUCAUCGAG |
| siRNA 1039 | 1039 | UCGAUGAAACACCAGAUGG | 7393 | CCAUCUGGUGUUUCAUCGA |
| siRNA 1040 | 1040 | CGAUGAAACACCAGAUGGA | 7394 | UCCAUCUGGUGUUUCAUCG |
| siRNA 1041 | 1041 | GAUGAAACACCAGAUGGAC | 7395 | GUCCAUCUGGUGUUUCAUC |
| siRNA 1042 | 1042 | AUGAAACACCAGAUGGACG | 7396 | CGUCCAUCUGGUGUUUCAU |
| siRNA 1043 | 1043 | UGAAACACCAGAUGGACGG | 7397 | CCGUCCAUCUGGUGUUUCA |
| siRNA 1044 | 1044 | GAAACACCAGAUGGACGGA | 7398 | UCCGUCCAUCUGGUGUUUC |
| siRNA 1045 | 1045 | AAACACCAGAUGGACGGAA | 7399 | UUCCGUCCAUCUGGUGUUU |
| siRNA 1046 | 1046 | AACACCAGAUGGACGGAAA | 7400 | UUUCCGUCCAUCUGGUGUU |
| siRNA 1047 | 1047 | ACACCAGAUGGACGGAAAG | 7401 | CUUUCCGUCCAUCUGGUGU |
| siRNA 1048 | 1048 | CACCAGAUGGACGGAAAGA | 7402 | UCUUUCCGUCCAUCUGGUG |
| siRNA 1049 | 1049 | ACCAGAUGGACGGAAAGA | 7403 | AUCUUUCCGUCCAUCUGGU |
| | | U | | |
| siRNA 1050 | 1050 | CCAGAUGGACGGAAAGAU | 7404 | CAUCUUUCCGUCCAUCUGG |
| | | G | | |
| siRNA 1051 | 1051 | CAGAUGGACGGAAAGAUG | 7405 | ACAUCUUUCCGUCCAUCUG |
| | | U | | |
| siRNA 1052 | 1052 | AGAUGGACGGAAAGAUGU | 7406 | AACAUCUUUCCGUCCAUCU |
| | | U | | |
| siRNA 1053 | 1053 | GAUGGACGGAAAGAUGUU | 7407 | GAACAUCUUUCCGUCCAUC |
| | | C | | |
| siRNA 1054 | 1054 | AUGGACGGAAAGAUGUUC | 7408 | AGAACAUCUUUCCGUCCAU |
| | | U | | |
| siRNA 1055 | 1055 | UGGACGGAAAGAUGUUCU | 7409 | GAGAACAUCUUUCCGUCCA |
| | | C | | |
| siRNA 1056 | 1056 | GGACGGAAAGAUGUUCUC | 7410 | AGAGAACAUCUUUCCGUCC |
| | | U | | |
| siRNA 1057 | 1057 | GACGGAAAGAUGUUCUCU | 7411 | UAGAGAACAUCUUUCCGUC |
| | | A | | |
| siRNA 1058 | 1058 | ACGGAAAGAUGUUCUCUA | 7412 | AUAGAGAACAUCUUUCCGU |
| | | U | | |
| siRNA 1059 | 1059 | CGGAAAGAUGUUCUCUAU | 7413 | UAUAGAGAACAUCUUUCCG |
| | | A | | |
| siRNA 1060 | 1060 | GGAAAGAUGUUCUCUAUA | 7414 | CUAUAGAGAACAUCUUUCC |
| | | G | | |
| siRNA 1061 | 1061 | GAAAGAUGUUCUCUAUAG | 7415 | UCUAUAGAGAACAUCUUUC |
| | | A | | |
| siRNA 1062 | 1062 | AAAGAUGUUCUCUAUAGA | 7416 | CUCUAUAGAGAACAUCUUU |
| | | G | | |
| siRNA 1063 | 1063 | AAGAUGUUCUCUAUAGAG | 7417 | GCUCUAUAGAGAACAUCUU |
| | | C | | |
| siRNA 1064 | 1064 | AGAUGUUCUCUAUAGAGC | 7418 | AGCUCUAUAGAGAACAUCU |
| | | U | | |
| siRNA 1065 | 1065 | GAUGUUCUCUAUAGAGCU | 7419 | AAGCUCUAUAGAGAACAUC |
| | | U | | |
| siRNA 1066 | 1066 | AUGUUCUCUAUAGAGCUU | 7420 | AAAGCUCUAUAGAGAACA |
| | | U | | U |
| siRNA 1067 | 1067 | UGUUCUCUAUAGAGCUUU | 7421 | CAAAGCUCUAUAGAGAACA |
| | | G | | |
| siRNA 1068 | 1068 | GUUCUCUAUAGAGCUUUGC | 7422 | GCAAAGCUCUAUAGAGAAC |
| siRNA 1069 | 1069 | UUCUCUAUAGAGCUUUGCU | 7423 | AGCAAAGCUCUAUAGAGA |
| | | | | A |
| siRNA 1070 | 1070 | UCUCUAUAGAGCUUUGCUC | 7424 | GAGCAAAGCUCUAUAGAG |
| | | | | A |
| siRNA 1071 | 1071 | CUCUAUAGAGCUUUGCUCC | 7425 | GGAGCAAAGCUCUAUAGA |
| | | | | G |
| siRNA 1072 | 1072 | UCUAUAGAGCUUUGCUCCA | 7426 | UGGAGCAAAGCUCUAUAG |
| | | | | A |
| siRNA 1073 | 1073 | CUAUAGAGCUUUGCUCCAU | 7427 | AUGGAGCAAAGCUCUAUA |
| | | | | G |
| siRNA 1074 | 1074 | UAUAGAGCUUUGCUCCAUG | 7428 | CAUGGAGCAAAGCUCUAUA |
| siRNA 1075 | 1075 | AUAGAGCUUUGCUCCAUGG | 7429 | CCAUGGAGCAAAGCUCUAU |
| siRNA 1076 | 1076 | UAGAGCUUUGCUCCAUGGG | 7430 | CCCAUGGAGCAAAGCUCUA |
| siRNA 1077 | 1077 | AGAGCUUUGCUCCAUGGGC | 7431 | GCCCAUGGAGCAAAGCUCU |
| siRNA 1078 | 1078 | GAGCUUUGCUCCAUGGGCA | 7432 | UGCCCAUGGAGCAAAGCUC |
| siRNA 1079 | 1079 | AGCUUUGCUCCAUGGGCAU | 7433 | AUGCCCAUGGAGCAAAGCU |
| siRNA 1080 | 1080 | GCUUUGCUCCAUGGGCAUA | 7434 | UAUGCCCAUGGAGCAAAGC |
| siRNA 1081 | 1081 | CUUUGCUCCAUGGGCAUAU | 7435 | AUAUGCCCAUGGAGCAAAG |
| siRNA 1082 | 1082 | UUUGCUCCAUGGGCAUAUA | 7436 | UAUAUGCCCAUGGAGCAAA |
| siRNA 1083 | 1083 | UUGCUCCAUGGGCAUAUAG | 7437 | CUAUAUGCCCAUGGAGCAA |
| siRNA 1084 | 1084 | UGCUCCAUGGGCAUAUAGU | 7438 | ACUAUAUGCCCAUGGAGCA |
| siRNA 1085 | 1085 | GCUCCAUGGGCAUAUAGUU | 7439 | AACUAUAUGCCCAUGGAGC |
| siRNA 1086 | 1086 | CUCCAUGGGCAUAUAGUUG | 7440 | CAACUAUAUGCCCAUGGAG |
| siRNA 1087 | 1087 | UCCAUGGGCAUAUAGUUG | 7441 | UCAACUAUAUGCCCAUGGA |
| | | A | | |
| siRNA 1088 | 1088 | CCAUGGGCAUAUAGUUGA | 7442 | UUCAACUAUAUGCCCAUGG |
| | | A | | |
| siRNA 1089 | 1089 | CAUGGGCAUAUAGUUGAA | 7443 | AUUCAACUAUAUGCCCAUG |
| | | U | | |
| siRNA 1090 | 1090 | AUGGGCAUAUAGUUGAAU | 7444 | AAUUCAACUAUAUGCCCAU |
| | | U | | |
| siRNA 1091 | 1091 | UGGGCAUAUAGUUGAAUU | 7445 | UAAUUCAACUAUAUGCCCA |
| | | A | | |
| siRNA 1092 | 1092 | GGGCAUAUAGUUGAAUUA | 7446 | GUAAUUCAACUAUAUGCCC |
| | | C | | |
| siRNA 1093 | 1093 | GGCAUAUAGUUGAAUUAC | 7447 | AGUAAUUCAACUAUAUGCC |
| | | U | | |
| siRNA 1094 | 1094 | GCAUAUAGUUGAAUUACU | 7448 | AAGUAAUUCAACUAUAUG |
| | | U | | C |
| siRNA 1095 | 1095 | CAUAUAGUUGAAUUACUU | 7449 | GAAGUAAUUCAACUAUAU |
| | | C | | G |
| siRNA 1096 | 1096 | AUAUAGUUGAAUUACUUC | 7450 | CGAAGUAAUUCAACUAUA |
| | | G | | U |
| siRNA 1097 | 1097 | UAUAGUUGAAUUACUUCG | 7451 | UCGAAGUAAUUCAACUAU |
| | | A | | A |
| siRNA 1098 | 1098 | AUAGUUGAAUUACUUCGA | 7452 | GUCGAAGUAAUUCAACUA |
| | | C | | U |
| siRNA 1099 | 1099 | UAGUUGAAUUACUUCGAC | 7453 | UGUCGAAGUAAUUCAACU |
| | | A | | A |
| siRNA 1100 | 1100 | AGUUGAAUUACUUCGACA | 7454 | CUGUCGAAGUAAUUCAACU |
| | | G | | |
| siRNA 1101 | 1101 | GUUGAAUUACUUCGACAGC | 7455 | GCUGUCGAAGUAAUUCAAC |
| siRNA 1102 | 1102 | UUGAAUUACUUCGACAGCA | 7456 | UGCUGUCGAAGUAAUUCA |
| | | | | A |
| siRNA 1103 | 1103 | UGAAUUACUUCGACAGCAG | 7457 | CUGCUGUCGAAGUAAUUCA |
| siRNA 1104 | 1104 | GAAUUACUUCGACAGCAGC | 7458 | GCUGCUGUCGAAGUAAUUC |
| siRNA 1105 | 1105 | AAUUACUUCGACAGCAGCA | 7459 | UGCUGCUGUCGAAGUAAU |
| | | | | U |
| siRNA 1106 | 1106 | AUUACUUCGACAGCAGCAA | 7460 | UUGCUGCUGUCGAAGUAA |
| | | | | U |
| siRNA 1107 | 1107 | UUACUUCGACAGCAGCAAU | 7461 | AUUGCUGCUGUCGAAGUA |
| | | | | A |
| siRNA 1108 | 1108 | UACUUCGACAGCAGCAAUU | 7462 | AAUUGCUGCUGUCGAAGU |
| | | | | A |
| siRNA 1109 | 1109 | ACUUCGACAGCAGCAAUUC | 7463 | GAAUUGCUGCUGUCGAAG |
| | | | | U |
| siRNA 1110 | 1110 | CUUCGACAGCAGCAAUUCU | 7464 | AGAAUUGCUGCUGUCGAA |
| | | | | G |
| siRNA 1111 | 1111 | UUCGACAGCAGCAAUUCUU | 7465 | AAGAAUUGCUGCUGUCGA |
| | | | | A |
| siRNA 1112 | 1112 | UCGACAGCAGCAAUUCUUG | 7466 | CAAGAAUUGCUGCUGUCGA |
| siRNA 1113 | 1113 | CGACAGCAGCAAUUCUUGG | 7467 | CCAAGAAUUGCUGCUGUCG |
| siRNA 1114 | 1114 | GACAGCAGCAAUUCUUGGA | 7468 | UCCAAGAAUUGCUGCUGUC |
| siRNA 1115 | 1115 | ACAGCAGCAAUUCUUGGAG | 7469 | CUCCAAGAAUUGCUGCUGU |
| siRNA 1116 | 1116 | CAGCAGCAAUUCUUGGAGA | 7470 | UCUCCAAGAAUUGCUGCUG |
| siRNA 1117 | 1117 | AGCAGCAAUUCUUGGAGA | 7471 | AUCUCCAAGAAUUGCUGCU |
| | | U | | |
| siRNA 1118 | 1118 | GCAGCAAUUCUUGGAGAUC | 7472 | GAUCUCCAAGAAUUGCUGC |
| siRNA 1119 | 1119 | CAGCAAUUCUUGGAGAUCU | 7473 | AGAUCUCCAAGAAUUGCUG |
| siRNA 1120 | 1120 | AGCAAUUCUUGGAGAUCU | 7474 | AAGAUCUCCAAGAAUUGCU |
| | | U | | |
| siRNA 1121 | 1121 | GCAAUUCUUGGAGAUCUUC | 7475 | GAAGAUCUCCAAGAAUUGC |
| siRNA 1122 | 1122 | CAAUUCUUGGAGAUCUUCC | 7476 | GGAAGAUCUCCAAGAAUU |
| | | | | G |
| siRNA 1123 | 1123 | AAUUCUUGGAGAUCUUCCU | 7477 | AGGAAGAUCUCCAAGAAU |
| | | | | U |
| siRNA 1124 | 1124 | AUUCUUGGAGAUCUUCCUG | 7478 | CAGGAAGAUCUCCAAGAAU |
| siRNA 1125 | 1125 | UUCUUGGAGAUCUUCCUGG | 7479 | CCAGGAAGAUCUCCAAGAA |
| siRNA 1126 | 1126 | UCUUGGAGAUCUUCCUGGA | 7480 | UCCAGGAAGAUCUCCAAGA |
| siRNA 1127 | 1127 | CUUGGAGAUCUUCCUGGAA | 7481 | UUCCAGGAAGAUCUCCAAG |
| siRNA 1128 | 1128 | UUGGAGAUCUUCCUGGAA | 7482 | CUUCCAGGAAGAUCUCCAA |
| | | G | | |
| siRNA 1129 | 1129 | UGGAGAUCUUCCUGGAAG | 7483 | CCUUCCAGGAAGAUCUCCA |
| | | G | | |
| siRNA 1130 | 1130 | GGAGAUCUUCCUGGAAGGC | 7484 | GCCUUCCAGGAAGAUCUCC |
| siRNA 1131 | 1131 | GAGAUCUUCCUGGAAGGCA | 7485 | UGCCUUCCAGGAAGAUCUC |
| siRNA 1132 | 1132 | AGAUCUUCCUGGAAGGCAC | 7486 | GUGCCUUCCAGGAAGAUCU |
| siRNA 1133 | 1133 | GAUCUUCCUGGAAGGCACA | 7487 | UGUGCCUUCCAGGAAGAUC |
| siRNA 1134 | 1134 | AUCUUCCUGGAAGGCACAC | 7488 | GUGUGCCUUCCAGGAAGAU |
| siRNA 1135 | 1135 | UCUUCCUGGAAGGCACACG | 7489 | CGUGUGCCUUCCAGGAAGA |
| siRNA 1136 | 1136 | CUUCCUGGAAGGCACACGU | 7490 | ACGUGUGCCUUCCAGGAAG |
| siRNA 1137 | 1137 | UUCCUGGAAGGCACACGUU | 7491 | AACGUGUGCCUUCCAGGAA |
| siRNA 1138 | 1138 | UCCUGGAAGGCACACGUUC | 7492 | GAACGUGUGCCUUCCAGGA |
| siRNA 1139 | 1139 | CCUGGAAGGCACACGUUCU | 7493 | AGAACGUGUGCCUUCCAGG |
| siRNA 1140 | 1140 | CUGGAAGGCACACGUUCUA | 7494 | UAGAACGUGUGCCUUCCAG |
| siRNA 1141 | 1141 | UGGAAGGCACACGUUCUAG | 7495 | CUAGAACGUGUGCCUUCCA |
| siRNA 1142 | 1142 | GGAAGGCACACGUUCUAGG | 7496 | CCUAGAACGUGUGCCUUCC |
| siRNA 1143 | 1143 | GAAGGCACACGUUCUAGGA | 7497 | UCCUAGAACGUGUGCCUUC |
| siRNA 1144 | 1144 | AAGGCACACGUUCUAGGAG | 7498 | CUCCUAGAACGUGUGCCUU |
| siRNA 1145 | 1145 | AGGCACACGUUCUAGGAGU | 7499 | ACUCCUAGAACGUGUGCCU |
| siRNA 1146 | 1146 | GGCACACGUUCUAGGAGUG | 7500 | CACUCCUAGAACGUGUGCC |
| siRNA 1147 | 1147 | GCACACGUUCUAGGAGUGG | 7501 | CCACUCCUAGAACGUGUGC |
| siRNA 1148 | 1148 | CACACGUUCUAGGAGUGGA | 7502 | UCCACUCCUAGAACGUGUG |
| siRNA 1149 | 1149 | ACACGUUCUAGGAGUGGA | 7503 | UUCCACUCCUAGAACGUGU |
| | | A | | |
| siRNA 1150 | 1150 | CACGUUCUAGGAGUGGAA | 7504 | UUUCCACUCCUAGAACGUG |
| | | A | | |
| siRNA 1151 | 1151 | ACGUUCUAGGAGUGGAAA | 7505 | UUUUCCACUCCUAGAACGU |
| | | A | | |
| siRNA 1152 | 1152 | CGUUCUAGGAGUGGAAAA | 7506 | UUUUUCCACUCCUAGAACG |
| | | A | | |
| siRNA 1153 | 1153 | GUUCUAGGAGUGGAAAAA | 7507 | GUUUUUCCACUCCUAGAAC |
| | | C | | |
| siRNA 1154 | 1154 | UUCUAGGAGUGGAAAAAC | 7508 | GGUUUUUCCACUCCUAGAA |
| | | C | | |
| siRNA 1155 | 1155 | UCUAGGAGUGGAAAAACC | 7509 | AGGUUUUUCCACUCCUAGA |
| | | U | | |
| siRNA 1156 | 1156 | CUAGGAGUGGAAAAACCUC | 7510 | GAGGUUUUUCCACUCCUAG |
| siRNA 1157 | 1157 | UAGGAGUGGAAAAACCUC | 7511 | AGAGGUUUUUCCACUCCUA |
| | | U | | |
| siRNA 1158 | 1158 | AGGAGUGGAAAAACCUCU | 7512 | AAGAGGUUUUUCCACUCCU |
| | | U | | |
| siRNA 1159 | 1159 | GGAGUGGAAAAACCUCUU | 7513 | CAAGAGGUUUUUCCACUCC |
| | | G | | |
| siRNA 1160 | 1160 | GAGUGGAAAAACCUCUUG | 7514 | ACAAGAGGUUUUUCCACUC |
| | | U | | |
| siRNA 1161 | 1161 | AGUGGAAAAACCUCUUGU | 7515 | CACAAGAGGUUUUUCCACU |
| | | G | | |
| siRNA 1162 | 1162 | GUGGAAAAACCUCUUGUGC | 7516 | GCACAAGAGGUUUUUCCAC |
| siRNA 1163 | 1163 | UGGAAAAACCUCUUGUGCU | 7517 | AGCACAAGAGGUUUUUCCA |
| siRNA 1164 | 1164 | GGAAAAACCUCUUGUGCUC | 7518 | GAGCACAAGAGGUUUUUCC |
| siRNA 1165 | 1165 | GAAAAACCUCUUGUGCUCG | 7519 | CGAGCACAAGAGGUUUUUC |
| siRNA 1166 | 1166 | AAAAACCUCUUGUGCUCGG | 7520 | CCGAGCACAAGAGGUUUUU |
| siRNA 1167 | 1167 | AAAACCUCUUGUGCUCGGG | 7521 | CCCGAGCACAAGAGGUUUU |
| siRNA 1168 | 1168 | AAACCUCUUGUGCUCGGGC | 7522 | GCCCGAGCACAAGAGGUUU |
| siRNA 1169 | 1169 | AACCUCUUGUGCUCGGGCA | 7523 | UGCCCGAGCACAAGAGGUU |
| siRNA 1170 | 1170 | ACCUCUUGUGCUCGGGCAG | 7524 | CUGCCCGAGCACAAGAGGU |
| siRNA 1171 | 1171 | CCUCUUGUGCUCGGGCAGG | 7525 | CCUGCCCGAGCACAAGAGG |
| siRNA 1172 | 1172 | CUCUUGUGCUCGGGCAGGA | 7526 | UCCUGCCCGAGCACAAGAG |
| siRNA 1173 | 1173 | UCUUGUGCUCGGGCAGGAC | 7527 | GUCCUGCCCGAGCACAAGA |
| siRNA 1174 | 1174 | CUUGUGCUCGGGCAGGACU | 7528 | AGUCCUGCCCGAGCACAAG |
| siRNA 1175 | 1175 | UUGUGCUCGGGCAGGACUU | 7529 | AAGUCCUGCCCGAGCACAA |
| siRNA 1176 | 1176 | UGUGCUCGGGCAGGACUUU | 7530 | AAAGUCCUGCCCGAGCACA |
| siRNA 1177 | 1177 | GUGCUCGGGCAGGACUUUU | 7531 | AAAAGUCCUGCCCGAGCAC |
| siRNA 1178 | 1178 | UGCUCGGGCAGGACUUUUG | 7532 | CAAAAGUCCUGCCCGAGCA |
| siRNA 1179 | 1179 | GCUCGGGCAGGACUUUUGU | 7533 | ACAAAAGUCCUGCCCGAGC |
| siRNA 1180 | 1180 | CUCGGGCAGGACUUUUGUC | 7534 | GACAAAAGUCCUGCCCGAG |
| siRNA 1181 | 1181 | UCGGGCAGGACUUUUGUCA | 7535 | UGACAAAAGUCCUGCCCGA |
| siRNA 1182 | 1182 | CGGGCAGGACUUUUGUCAG | 7536 | CUGACAAAAGUCCUGCCCG |
| siRNA 1183 | 1183 | GGGCAGGACUUUUGUCAG | 7537 | ACUGACAAAAGUCCUGCCC |
| | | U | | |
| siRNA 1184 | 1184 | GGCAGGACUUUUGUCAGU | 7538 | AACUGACAAAAGUCCUGCC |
| | | U | | |
| siRNA 1185 | 1185 | GCAGGACUUUUGUCAGUU | 7539 | CAACUGACAAAAGUCCUGC |
| | | G | | |
| siRNA 1186 | 1186 | CAGGACUUUUGUCAGUUG | 7540 | ACAACUGACAAAAGUCCUG |
| | | U | | |
| siRNA 1187 | 1187 | AGGACUUUUGUCAGUUGU | 7541 | CACAACUGACAAAAGUCCU |
| | | G | | |
| siRNA 1188 | 1188 | GGACUUUUGUCAGUUGUG | 7542 | CCACAACUGACAAAAGUCC |
| | | G | | |
| siRNA 1189 | 1189 | GACUUUUGUCAGUUGUGG | 7543 | ACCACAACUGACAAAAGUC |
| | | U | | |
| siRNA 1190 | 1190 | ACUUUUGUCAGUUGUGGU | 7544 | UACCACAACUGACAAAAGU |
| | | A | | |
| siRNA 1191 | 1191 | CUUUUGUCAGUUGUGGUA | 7545 | CUACCACAACUGACAAAAG |
| | | G | | |
| siRNA 1192 | 1192 | UUUUGUCAGUUGUGGUAG | 7546 | UCUACCACAACUGACAAAA |
| | | A | | |
| siRNA 1193 | 1193 | UUUGUCAGUUGUGGUAGA | 7547 | AUCUACCACAACUGACAAA |
| | | U | | |
| siRNA 1194 | 1194 | UUGUCAGUUGUGGUAGAU | 7548 | UAUCUACCACAACUGACAA |
| | | A | | |
| siRNA 1195 | 1195 | UGUCAGUUGUGGUAGAUA | 7549 | GUAUCUACCACAACUGACA |
| | | C | | |
| siRNA 1196 | 1196 | GUCAGUUGUGGUAGAUAC | 7550 | AGUAUCUACCACAACUGAC |
| | | U | | |
| siRNA 1197 | 1197 | UCAGUUGUGGUAGAUACU | 7551 | GAGUAUCUACCACAACUGA |
| | | C | | |
| siRNA 1198 | 1198 | CAGUUGUGGUAGAUACUC | 7552 | AGAGUAUCUACCACAACUG |
| | | U | | |
| siRNA 1199 | 1199 | AGUUGUGGUAGAUACUCU | 7553 | CAGAGUAUCUACCACAACU |
| | | G | | |
| siRNA 1200 | 1200 | GUUGUGGUAGAUACUCUG | 7554 | ACAGAGUAUCUACCACAAC |
| | | U | | |
| siRNA 1201 | 1201 | UUGUGGUAGAUACUCUGU | 7555 | GACAGAGUAUCUACCACAA |
| | | C | | |
| siRNA 1202 | 1202 | UGUGGUAGAUACUCUGUC | 7556 | AGACAGAGUAUCUACCACA |
| | | U | | |
| siRNA 1203 | 1203 | GUGGUAGAUACUCUGUCU | 7557 | UAGACAGAGUAUCUACCAC |
| | | A | | |
| siRNA 1204 | 1204 | UGGUAGAUACUCUGUCUAC | 7558 | GUAGACAGAGUAUCUACCA |
| siRNA 1205 | 1205 | GGUAGAUACUCUGUCUACC | 7559 | GGUAGACAGAGUAUCUACC |
| siRNA 1206 | 1206 | GUAGAUACUCUGUCUACCA | 7560 | UGGUAGACAGAGUAUCUA |
| | | | | C |
| siRNA 1207 | 1207 | UAGAUACUCUGUCUACCAA | 7561 | UUGGUAGACAGAGUAUCU |
| | | | | A |
| siRNA 1208 | 1208 | AGAUACUCUGUCUACCAAU | 7562 | AUUGGUAGACAGAGUAUC |
| | | | | U |
| siRNA 1209 | 1209 | GAUACUCUGUCUACCAAUG | 7563 | CAUUGGUAGACAGAGUAU |
| | | | | C |
| siRNA 1210 | 1210 | AUACUCUGUCUACCAAUGU | 7564 | ACAUUGGUAGACAGAGUA |
| | | | | U |
| siRNA 1211 | 1211 | UACUCUGUCUACCAAUGUC | 7565 | GACAUUGGUAGACAGAGU |
| | | | | A |
| siRNA 1212 | 1212 | ACUCUGUCUACCAAUGUCA | 7566 | UGACAUUGGUAGACAGAG |
| | | | | U |
| siRNA 1213 | 1213 | CUCUGUCUACCAAUGUCAU | 7567 | AUGACAUUGGUAGACAGA |
| | | | | G |
| siRNA 1214 | 1214 | UCUGUCUACCAAUGUCAUC | 7568 | GAUGACAUUGGUAGACAG |
| | | | | A |
| siRNA 1215 | 1215 | CUGUCUACCAAUGUCAUCC | 7569 | GGAUGACAUUGGUAGACA |
| | | | | G |
| siRNA 1216 | 1216 | UGUCUACCAAUGUCAUCCC | 7570 | GGGAUGACAUUGGUAGAC |
| | | | | A |
| siRNA 1217 | 1217 | GUCUACCAAUGUCAUCCCA | 7571 | UGGGAUGACAUUGGUAGA |
| | | | | C |
| siRNA 1218 | 1218 | UCUACCAAUGUCAUCCCAG | 7572 | CUGGGAUGACAUUGGUAG |
| | | | | A |
| siRNA 1219 | 1219 | CUACCAAUGUCAUCCCAGA | 7573 | UCUGGGAUGACAUUGGUA |
| | | | | G |
| siRNA 1220 | 1220 | UACCAAUGUCAUCCCAGAC | 7574 | GUCUGGGAUGACAUUGGU |
| | | | | A |
| siRNA 1221 | 1221 | ACCAAUGUCAUCCCAGACA | 7575 | UGUCUGGGAUGACAUUGG |
| | | | | U |
| siRNA 1222 | 1222 | CCAAUGUCAUCCCAGACAU | 7576 | AUGUCUGGGAUGACAUUG |
| | | | | G |
| siRNA 1223 | 1223 | CAAUGUCAUCCCAGACAUC | 7577 | GAUGUCUGGGAUGACAUU |
| | | | | G |
| siRNA 1224 | 1224 | AAUGUCAUCCCAGACAUCU | 7578 | AGAUGUCUGGGAUGACAU |
| | | | | U |
| siRNA 1225 | 1225 | AUGUCAUCCCAGACAUCUU | 7579 | AAGAUGUCUGGGAUGACA |
| | | | | U |
| siRNA 1226 | 1226 | UGUCAUCCCAGACAUCUUG | 7580 | CAAGAUGUCUGGGAUGAC |
| | | | | A |
| siRNA 1227 | 1227 | GUCAUCCCAGACAUCUUGA | 7581 | UCAAGAUGUCUGGGAUGA |
| | | | | C |
| siRNA 1228 | 1228 | UCAUCCCAGACAUCUUGAU | 7582 | AUCAAGAUGUCUGGGAUG |
| | | | | A |
| siRNA 1229 | 1229 | CAUCCCAGACAUCUUGAUA | 7583 | UAUCAAGAUGUCUGGGAU |
| | | | | G |
| siRNA 1230 | 1230 | AUCCCAGACAUCUUGAUAA | 7584 | UUAUCAAGAUGUCUGGGA |
| | | | | U |
| siRNA 1231 | 1231 | UCCCAGACAUCUUGAUAAU | 7585 | AUUAUCAAGAUGUCUGGG |
| | | | | A |
| siRNA 1232 | 1232 | CCCAGACAUCUUGAUAAUA | 7586 | UAUUAUCAAGAUGUCUGG |
| | | | | G |
| siRNA 1233 | 1233 | CCAGACAUCUUGAUAAUAC | 7587 | GUAUUAUCAAGAUGUCUG |
| | | | | G |
| siRNA 1234 | 1234 | CAGACAUCUUGAUAAUACC | 7588 | GGUAUUAUCAAGAUGUCU |
| | | | | G |
| siRNA 1235 | 1235 | AGACAUCUUGAUAAUACCU | 7589 | AGGUAUUAUCAAGAUGUC |
| | | | | U |
| siRNA 1236 | 1236 | GACAUCUUGAUAAUACCUG | 7590 | CAGGUAUUAUCAAGAUGU |
| | | | | C |
| siRNA 1237 | 1237 | ACAUCUUGAUAAUACCUGU | 7591 | ACAGGUAUUAUCAAGAUG |
| | | | | U |
| siRNA 1238 | 1238 | CAUCUUGAUAAUACCUGUU | 7592 | AACAGGUAUUAUCAAGAU |
| | | | | G |
| siRNA 1239 | 1239 | AUCUUGAUAAUACCUGUU | 7593 | CAACAGGUAUUAUCAAGA |
| | | G | | U |
| siRNA 1240 | 1240 | UCUUGAUAAUACCUGUUG | 7594 | CCAACAGGUAUUAUCAAGA |
| | | G | | |
| siRNA 1241 | 1241 | CUUGAUAAUACCUGUUGG | 7595 | UCCAACAGGUAUUAUCAAG |
| | | A | | |
| siRNA 1242 | 1242 | UUGAUAAUACCUGUUGGA | 7596 | UUCCAACAGGUAUUAUCAA |
| | | A | | |
| siRNA 1243 | 1243 | UGAUAAUACCUGUUGGAA | 7597 | AUUCCAACAGGUAUUAUCA |
| | | U | | |
| siRNA 1244 | 1244 | GAUAAUACCUGUUGGAAU | 7598 | GAUUCCAACAGGUAUUAUC |
| | | C | | |
| siRNA 1245 | 1245 | AUAAUACCUGUUGGAAUC | 7599 | AGAUUCCAACAGGUAUUA |
| | | U | | U |
| siRNA 1246 | 1246 | UAAUACCUGUUGGAAUCUC | 7600 | GAGAUUCCAACAGGUAUU |
| | | | | A |
| siRNA 1247 | 1247 | AAUACCUGUUGGAAUCUCC | 7601 | GGAGAUUCCAACAGGUAU |
| | | | | U |
| siRNA 1248 | 1248 | AUACCUGUUGGAAUCUCCU | 7602 | AGGAGAUUCCAACAGGUA |
| | | | | U |
| siRNA 1249 | 1249 | UACCUGUUGGAAUCUCCUA | 7603 | UAGGAGAUUCCAACAGGU |
| | | | | A |
| siRNA 1250 | 1250 | ACCUGUUGGAAUCUCCUAU | 7604 | AUAGGAGAUUCCAACAGG |
| | | | | U |
| siRNA 1251 | 1251 | CCUGUUGGAAUCUCCUAUG | 7605 | CAUAGGAGAUUCCAACAGG |
| siRNA 1252 | 1252 | CUGUUGGAAUCUCCUAUGA | 7606 | UCAUAGGAGAUUCCAACAG |
| siRNA 1253 | 1253 | UGUUGGAAUCUCCUAUGA | 7607 | AUCAUAGGAGAUUCCAACA |
| | | U | | |
| siRNA 1254 | 1254 | GUUGGAAUCUCCUAUGAUC | 7608 | GAUCAUAGGAGAUUCCAAC |
| siRNA 1255 | 1255 | UUGGAAUCUCCUAUGAUCG | 7609 | CGAUCAUAGGAGAUUCCAA |
| siRNA 1256 | 1256 | UGGAAUCUCCUAUGAUCGC | 7610 | GCGAUCAUAGGAGAUUCCA |
| siRNA 1257 | 1257 | GGAAUCUCCUAUGAUCGCA | 7611 | UGCGAUCAUAGGAGAUUCC |
| siRNA 1258 | 1258 | GAAUCUCCUAUGAUCGCAU | 7612 | AUGCGAUCAUAGGAGAUU |
| | | | | C |
| siRNA 1259 | 1259 | AAUCUCCUAUGAUCGCAUU | 7613 | AAUGCGAUCAUAGGAGAU |
| | | | | U |
| siRNA 1260 | 1260 | AUCUCCUAUGAUCGCAUUA | 7614 | UAAUGCGAUCAUAGGAGA |
| | | | | U |
| siRNA 1261 | 1261 | UCUCCUAUGAUCGCAUUAU | 7615 | AUAAUGCGAUCAUAGGAG |
| | | | | A |
| siRNA 1262 | 1262 | CUCCUAUGAUCGCAUUAUC | 7616 | GAUAAUGCGAUCAUAGGA |
| | | | | G |
| siRNA 1263 | 1263 | UCCUAUGAUCGCAUUAUCG | 7617 | CGAUAAUGCGAUCAUAGG |
| | | | | A |
| siRNA 1264 | 1264 | CCUAUGAUCGCAUUAUCGA | 7618 | UCGAUAAUGCGAUCAUAG |
| | | | | G |
| siRNA 1265 | 1265 | CUAUGAUCGCAUUAUCGAA | 7619 | UUCGAUAAUGCGAUCAUA |
| | | | | G |
| siRNA 1266 | 1266 | UAUGAUCGCAUUAUCGAA | 7620 | CUUCGAUAAUGCGAUCAUA |
| | | G | | |
| siRNA 1267 | 1267 | AUGAUCGCAUUAUCGAAG | 7621 | CCUUCGAUAAUGCGAUCAU |
| | | G | | |
| siRNA 1268 | 1268 | UGAUCGCAUUAUCGAAGG | 7622 | ACCUUCGAUAAUGCGAUCA |
| | | U | | |
| siRNA 1269 | 1269 | GAUCGCAUUAUCGAAGGUC | 7623 | GACCUUCGAUAAUGCGAUC |
| siRNA 1270 | 1270 | AUCGCAUUAUCGAAGGUCA | 7624 | UGACCUUCGAUAAUGCGAU |
| siRNA 1271 | 1271 | UCGCAUUAUCGAAGGUCAC | 7625 | GUGACCUUCGAUAAUGCGA |
| siRNA 1272 | 1272 | CGCAUUAUCGAAGGUCACU | 7626 | AGUGACCUUCGAUAAUGCG |
| siRNA 1273 | 1273 | GCAUUAUCGAAGGUCACUA | 7627 | UAGUGACCUUCGAUAAUGC |
| siRNA 1274 | 1274 | CAUUAUCGAAGGUCACUAC | 7628 | GUAGUGACCUUCGAUAAU |
| | | | | G |
| siRNA 1275 | 1275 | AUUAUCGAAGGUCACUACA | 7629 | UGUAGUGACCUUCGAUAA |
| | | | | U |
| siRNA 1276 | 1276 | UUAUCGAAGGUCACUACAA | 7630 | UUGUAGUGACCUUCGAUA |
| | | | | A |
| siRNA 1277 | 1277 | UAUCGAAGGUCACUACAAU | 7631 | AUUGUAGUGACCUUCGAU |
| | | | | A |
| siRNA 1278 | 1278 | AUCGAAGGUCACUACAAUG | 7632 | CAUUGUAGUGACCUUCGAU |
| siRNA 1279 | 1279 | UCGAAGGUCACUACAAUGG | 7633 | CCAUUGUAGUGACCUUCGA |
| siRNA 1280 | 1280 | CGAAGGUCACUACAAUGGU | 7634 | ACCAUUGUAGUGACCUUCG |
| siRNA 1281 | 1281 | GAAGGUCACUACAAUGGU | 7635 | CACCAUUGUAGUGACCUUC |
| | | G | | |
| siRNA 1282 | 1282 | AAGGUCACUACAAUGGUG | 7636 | UCACCAUUGUAGUGACCUU |
| | | A | | |
| siRNA 1283 | 1283 | AGGUCACUACAAUGGUGA | 7637 | UUCACCAUUGUAGUGACCU |
| | | A | | |
| siRNA 1284 | 1284 | GGUCACUACAAUGGUGAAC | 7638 | GUUCACCAUUGUAGUGACC |
| siRNA 1285 | 1285 | GUCACUACAAUGGUGAACA | 7639 | UGUUCACCAUUGUAGUGAC |
| siRNA 1286 | 1286 | UCACUACAAUGGUGAACAA | 7640 | UUGUUCACCAUUGUAGUG |
| | | | | A |
| siRNA 1287 | 1287 | CACUACAAUGGUGAACAAC | 7641 | GUUGUUCACCAUUGUAGU |
| | | | | G |
| siRNA 1288 | 1288 | ACUACAAUGGUGAACAACU | 7642 | AGUUGUUCACCAUUGUAG |
| | | | | U |
| siRNA 1289 | 1289 | CUACAAUGGUGAACAACUG | 7643 | CAGUUGUUCACCAUUGUAG |
| siRNA 1290 | 1290 | UACAAUGGUGAACAACUG | 7644 | CCAGUUGUUCACCAUUGUA |
| | | G | | |
| siRNA 1291 | 1291 | ACAAUGGUGAACAACUGG | 7645 | CCCAGUUGUUCACCAUUGU |
| | | G | | |
| siRNA 1292 | 1292 | CAAUGGUGAACAACUGGGC | 7646 | GCCCAGUUGUUCACCAUUG |
| siRNA 1293 | 1293 | AAUGGUGAACAACUGGGC | 7647 | UGCCCAGUUGUUCACCAUU |
| | | A | | |
| siRNA 1294 | 1294 | AUGGUGAACAACUGGGCA | 7648 | UUGCCCAGUUGUUCACCAU |
| | | A | | |
| siRNA 1295 | 1295 | UGGUGAACAACUGGGCAA | 7649 | UUUGCCCAGUUGUUCACCA |
| | | A | | |
| siRNA 1296 | 1296 | GGUGAACAACUGGGCAAAC | 7650 | GUUUGCCCAGUUGUUCACC |
| siRNA 1297 | 1297 | GUGAACAACUGGGCAAACC | 7651 | GGUUUGCCCAGUUGUUCAC |
| siRNA 1298 | 1298 | UGAACAACUGGGCAAACCU | 7652 | AGGUUUGCCCAGUUGUUCA |
| siRNA 1299 | 1299 | GAACAACUGGGCAAACCUA | 7653 | UAGGUUUGCCCAGUUGUUC |
| siRNA 1300 | 1300 | AACAACUGGGCAAACCUAA | 7654 | UUAGGUUUGCCCAGUUGU |
| | | | | U |
| siRNA 1301 | 1301 | ACAACUGGGCAAACCUAAG | 7655 | CUUAGGUUUGCCCAGUUGU |
| siRNA 1302 | 1302 | CAACUGGGCAAACCUAAGA | 7656 | UCUUAGGUUUGCCCAGUUG |
| siRNA 1303 | 1303 | AACUGGGCAAACCUAAGAA | 7657 | UUCUUAGGUUUGCCCAGUU |
| siRNA 1304 | 1304 | ACUGGGCAAACCUAAGAAG | 7658 | CUUCUUAGGUUUGCCCAGU |
| siRNA 1305 | 1305 | CUGGGCAAACCUAAGAAGA | 7659 | UCUUCUUAGGUUUGCCCAG |
| siRNA 1306 | 1306 | UGGGCAAACCUAAGAAGA | 7660 | UUCUUCUUAGGUUUGCCCA |
| | | A | | |
| siRNA 1307 | 1307 | GGGCAAACCUAAGAAGAA | 7661 | AUUCUUCUUAGGUUUGCCC |
| | | U | | |
| siRNA 1308 | 1308 | GGCAAACCUAAGAAGAAU | 7662 | CAUUCUUCUUAGGUUUGCC |
| | | G | | |
| siRNA 1309 | 1309 | GCAAACCUAAGAAGAAUG | 7663 | UCAUUCUUCUUAGGUUUGC |
| | | A | | |
| siRNA 1310 | 1310 | CAAACCUAAGAAGAAUGA | 7664 | CUCAUUCUUCUUAGGUUUG |
| | | G | | |
| siRNA 1311 | 1311 | AAACCUAAGAAGAAUGAG | 7665 | UCUCAUUCUUCUUAGGUUU |
| | | A | | |
| siRNA 1312 | 1312 | AACCUAAGAAGAAUGAGA | 7666 | CUCUCAUUCUUCUUAGGUU |
| | | G | | |
| siRNA 1313 | 1313 | ACCUAAGAAGAAUGAGAG | 7667 | GCUCUCAUUCUUCUUAGGU |
| | | C | | |
| siRNA 1314 | 1314 | CCUAAGAAGAAUGAGAGCC | 7668 | GGCUCUCAUUCUUCUUAGG |
| siRNA 1315 | 1315 | CUAAGAAGAAUGAGAGCC | 7669 | AGGCUCUCAUUCUUCUUAG |
| | | U | | |
| siRNA 1316 | 1316 | UAAGAAGAAUGAGAGCCU | 7670 | CAGGCUCUCAUUCUUCUUA |
| | | G | | |
| siRNA 1317 | 1317 | AAGAAGAAUGAGAGCCUG | 7671 | ACAGGCUCUCAUUCUUCUU |
| | | U | | |
| siRNA 1318 | 1318 | AGAAGAAUGAGAGCCUGU | 7672 | CACAGGCUCUCAUUCUUCU |
| | | G | | |
| siRNA 1319 | 1319 | GAAGAAUGAGAGCCUGUG | 7673 | CCACAGGCUCUCAUUCUUC |
| | | G | | |
| siRNA 1320 | 1320 | AAGAAUGAGAGCCUGUGG | 7674 | UCCACAGGCUCUCAUUCUU |
| | | A | | |
| siRNA 1321 | 1321 | AGAAUGAGAGCCUGUGGA | 7675 | CUCCACAGGCUCUCAUUCU |
| | | G | | |
| siRNA 1322 | 1322 | GAAUGAGAGCCUGUGGAG | 7676 | ACUCCACAGGCUCUCAUUC |
| | | U | | |
| siRNA 1323 | 1323 | AAUGAGAGCCUGUGGAGU | 7677 | CACUCCACAGGCUCUCAUU |
| | | G | | |
| siRNA 1324 | 1324 | AUGAGAGCCUGUGGAGUG | 7678 | ACACUCCACAGGCUCUCAU |
| | | U | | |
| siRNA 1325 | 1325 | UGAGAGCCUGUGGAGUGU | 7679 | UACACUCCACAGGCUCUCA |
| | | A | | |
| siRNA 1326 | 1326 | GAGAGCCUGUGGAGUGUA | 7680 | CUACACUCCACAGGCUCUC |
| | | G | | |
| siRNA 1327 | 1327 | AGAGCCUGUGGAGUGUAG | 7681 | GCUACACUCCACAGGCUCU |
| | | C | | |
| siRNA 1328 | 1328 | GAGCCUGUGGAGUGUAGC | 7682 | UGCUACACUCCACAGGCUC |
| | | A | | |
| siRNA 1329 | 1329 | AGCCUGUGGAGUGUAGCA | 7683 | UUGCUACACUCCACAGGCU |
| | | A | | |
| siRNA 1330 | 1330 | GCCUGUGGAGUGUAGCAA | 7684 | CUUGCUACACUCCACAGGC |
| | | G | | |
| siRNA 1331 | 1331 | CCUGUGGAGUGUAGCAAG | 7685 | UCUUGCUACACUCCACAGG |
| | | A | | |
| siRNA 1332 | 1332 | CUGUGGAGUGUAGCAAGA | 7686 | CUCUUGCUACACUCCACAG |
| | | G | | |
| siRNA 1333 | 1333 | UGUGGAGUGUAGCAAGAG | 7687 | CCUCUUGCUACACUCCACA |
| | | G | | |
| siRNA 1334 | 1334 | GUGGAGUGUAGCAAGAGG | 7688 | ACCUCUUGCUACACUCCAC |
| | | U | | |
| siRNA 1335 | 1335 | UGGAGUGUAGCAAGAGGU | 7689 | CACCUCUUGCUACACUCCA |
| | | G | | |
| siRNA 1336 | 1336 | GGAGUGUAGCAAGAGGUG | 7690 | ACACCUCUUGCUACACUCC |
| | | U | | |
| siRNA 1337 | 1337 | GAGUGUAGCAAGAGGUGU | 7691 | AACACCUCUUGCUACACUC |
| | | U | | |
| siRNA 1338 | 1338 | AGUGUAGCAAGAGGUGUU | 7692 | UAACACCUCUUGCUACACU |
| | | A | | |
| siRNA 1339 | 1339 | GUGUAGCAAGAGGUGUUA | 7693 | AUAACACCUCUUGCUACAC |
| | | U | | |
| siRNA 1340 | 1340 | UGUAGCAAGAGGUGUUAU | 7694 | AAUAACACCUCUUGCUACA |
| | | U | | |
| siRNA 1341 | 1341 | GUAGCAAGAGGUGUUAUU | 7695 | UAAUAACACCUCUUGCUAC |
| | | A | | |
| siRNA 1342 | 1342 | UAGCAAGAGGUGUUAUUA | 7696 | CUAAUAACACCUCUUGCUA |
| | | G | | |
| siRNA 1343 | 1343 | AGCAAGAGGUGUUAUUAG | 7697 | UCUAAUAACACCUCUUGCU |
| | | A | | |
| siRNA 1344 | 1344 | GCAAGAGGUGUUAUUAGA | 7698 | UUCUAAUAACACCUCUUGC |
| | | A | | |
| siRNA 1345 | 1345 | CAAGAGGUGUUAUUAGAA | 7699 | AUUCUAAUAACACCUCUUG |
| | | U | | |
| siRNA 1346 | 1346 | AAGAGGUGUUAUUAGAAU | 7700 | CAUUCUAAUAACACCUCUU |
| | | G | | |
| siRNA 1347 | 1347 | AGAGGUGUUAUUAGAAUG | 7701 | ACAUUCUAAUAACACCUCU |
| | | U | | |
| siRNA 1348 | 1348 | GAGGUGUUAUUAGAAUGU | 7702 | AACAUUCUAAUAACACCUC |
| | | U | | |
| siRNA 1349 | 1349 | AGGUGUUAUUAGAAUGUU | 7703 | UAACAUUCUAAUAACACCU |
| | | A | | |
| siRNA 1350 | 1350 | GGUGUUAUUAGAAUGUUA | 7704 | GUAACAUUCUAAUAACACC |
| | | C | | |
| siRNA 1351 | 1351 | GUGUUAUUAGAAUGUUAC | 7705 | CGUAACAUUCUAAUAACAC |
| | | G | | |
| siRNA 1352 | 1352 | UGUUAUUAGAAUGUUACG | 7706 | UCGUAACAUUCUAAUAACA |
| | | A | | |
| siRNA 1353 | 1353 | GUUAUUAGAAUGUUACGA | 7707 | UUCGUAACAUUCUAAUAAC |
| | | A | | |
| siRNA 1354 | 1354 | UUAUUAGAAUGUUACGAA | 7708 | UUUCGUAACAUUCUAAUA |
| | | A | | A |
| siRNA 1355 | 1355 | UAUUAGAAUGUUACGAAA | 7709 | UUUUCGUAACAUUCUAAU |
| | | A | | A |
| siRNA 1356 | 1356 | AUUAGAAUGUUACGAAAA | 7710 | UUUUUCGUAACAUUCUAA |
| | | A | | U |
| siRNA 1357 | 1357 | UUAGAAUGUUACGAAAAA | 7711 | UUUUUUCGUAACAUUCUA |
| | | A | | A |
| siRNA 1358 | 1358 | UAGAAUGUUACGAAAAAA | 7712 | GUUUUUUCGUAACAUUCU |
| | | C | | A |
| siRNA 1359 | 1359 | AGAAUGUUACGAAAAAAC | 7713 | AGUUUUUUCGUAACAUUC |
| | | U | | U |
| siRNA 1360 | 1360 | GAAUGUUACGAAAAAACU | 7714 | UAGUUUUUUCGUAACAUU |
| | | A | | C |
| siRNA 1361 | 1361 | AAUGUUACGAAAAAACUA | 7715 | AUAGUUUUUUCGUAACAU |
| | | U | | U |
| siRNA 1362 | 1362 | AUGUUACGAAAAAACUAU | 7716 | CAUAGUUUUUUCGUAACA |
| | | G | | U |
| siRNA 1363 | 1363 | UGUUACGAAAAAACUAUG | 7717 | CCAUAGUUUUUUCGUAACA |
| | | G | | |
| siRNA 1364 | 1364 | GUUACGAAAAAACUAUGG | 7718 | ACCAUAGUUUUUUCGUAAC |
| | | U | | |
| siRNA 1365 | 1365 | UUACGAAAAAACUAUGGU | 7719 | AACCAUAGUUUUUUCGUA |
| | | U | | A |
| siRNA 1366 | 1366 | UACGAAAAAACUAUGGUU | 7720 | CAACCAUAGUUUUUUCGUA |
| | | G | | |
| siRNA 1367 | 1367 | ACGAAAAAACUAUGGUUG | 7721 | ACAACCAUAGUUUUUUCGU |
| | | U | | |
| siRNA 1368 | 1368 | CGAAAAAACUAUGGUUGU | 7722 | CACAACCAUAGUUUUUUCG |
| | | G | | |
| siRNA 1369 | 1369 | GAAAAAACUAUGGUUGUG | 7723 | ACACAACCAUAGUUUUUUC |
| | | U | | |
| siRNA 1370 | 1370 | AAAAAACUAUGGUUGUGU | 7724 | GACACAACCAUAGUUUUUU |
| | | C | | |
| siRNA 1371 | 1371 | AAAAACUAUGGUUGUGUC | 7725 | GGACACAACCAUAGUUUUU |
| | | C | | |
| siRNA 1372 | 1372 | AAAACUAUGGUUGUGUCC | 7726 | CGGACACAACCAUAGUUUU |
| | | G | | |
| siRNA 1373 | 1373 | AAACUAUGGUUGUGUCCG | 7727 | UCGGACACAACCAUAGUUU |
| | | A | | |
| siRNA 1374 | 1374 | AACUAUGGUUGUGUCCGA | 7728 | CUCGGACACAACCAUAGUU |
| | | G | | |
| siRNA 1375 | 1375 | ACUAUGGUUGUGUCCGAG | 7729 | ACUCGGACACAACCAUAGU |
| | | U | | |
| siRNA 1376 | 1376 | CUAUGGUUGUGUCCGAGU | 7730 | CACUCGGACACAACCAUAG |
| | | G | | |
| siRNA 1377 | 1377 | UAUGGUUGUGUCCGAGUG | 7731 | CCACUCGGACACAACCAUA |
| | | G | | |
| siRNA 1378 | 1378 | AUGGUUGUGUCCGAGUGG | 7732 | UCCACUCGGACACAACCAU |
| | | A | | |
| siRNA 1379 | 1379 | UGGUUGUGUCCGAGUGGA | 7733 | AUCCACUCGGACACAACCA |
| | | U | | |
| siRNA 1380 | 1380 | GGUUGUGUCCGAGUGGAU | 7734 | AAUCCACUCGGACACAACC |
| | | U | | |
| siRNA 1381 | 1381 | GUUGUGUCCGAGUGGAUU | 7735 | AAAUCCACUCGGACACAAC |
| | | U | | |
| siRNA 1382 | 1382 | UUGUGUCCGAGUGGAUUU | 7736 | AAAAUCCACUCGGACACAA |
| | | U | | |
| siRNA 1383 | 1383 | UGUGUCCGAGUGGAUUUU | 7737 | CAAAAUCCACUCGGACACA |
| | | G | | |
| siRNA 1384 | 1384 | GUGUCCGAGUGGAUUUUG | 7738 | GCAAAAUCCACUCGGACAC |
| | | C | | |
| siRNA 1385 | 1385 | UGUCCGAGUGGAUUUUGC | 7739 | UGCAAAAUCCACUCGGACA |
| | | A | | |
| siRNA 1386 | 1386 | GUCCGAGUGGAUUUUGCAC | 7740 | GUGCAAAAUCCACUCGGAC |
| siRNA 1387 | 1387 | UCCGAGUGGAUUUUGCACA | 7741 | UGUGCAAAAUCCACUCGGA |
| siRNA 1388 | 1388 | CCGAGUGGAUUUUGCACAG | 7742 | CUGUGCAAAAUCCACUCGG |
| siRNA 1389 | 1389 | CGAGUGGAUUUUGCACAGC | 7743 | GCUGUGCAAAAUCCACUCG |
| siRNA 1390 | 1390 | GAGUGGAUUUUGCACAGCC | 7744 | GGCUGUGCAAAAUCCACUC |
| siRNA 1391 | 1391 | AGUGGAUUUUGCACAGCCA | 7745 | UGGCUGUGCAAAAUCCACU |
| siRNA 1392 | 1392 | GUGGAUUUUGCACAGCCAU | 7746 | AUGGCUGUGCAAAAUCCAC |
| siRNA 1393 | 1393 | UGGAUUUUGCACAGCCAUU | 7747 | AAUGGCUGUGCAAAAUCCA |
| siRNA 1394 | 1394 | GGAUUUUGCACAGCCAUUU | 7748 | AAAUGGCUGUGCAAAAUCC |
| siRNA 1395 | 1395 | GAUUUUGCACAGCCAUUUU | 7749 | AAAAUGGCUGUGCAAAAU |
| | | | | C |
| siRNA 1396 | 1396 | AUUUUGCACAGCCAUUUUC | 7750 | GAAAAUGGCUGUGCAAAA |
| | | | | U |
| siRNA 1397 | 1397 | UUUUGCACAGCCAUUUUCC | 7751 | GGAAAAUGGCUGUGCAAA |
| | | | | A |
| siRNA 1398 | 1398 | UUUGCACAGCCAUUUUCCU | 7752 | AGGAAAAUGGCUGUGCAA |
| | | | | A |
| siRNA 1399 | 1399 | UUGCACAGCCAUUUUCCUU | 7753 | AAGGAAAAUGGCUGUGCA |
| | | | | A |
| siRNA 1400 | 1400 | UGCACAGCCAUUUUCCUUA | 7754 | UAAGGAAAAUGGCUGUGC |
| | | | | A |
| siRNA 1401 | 1401 | GCACAGCCAUUUUCCUUAA | 7755 | UUAAGGAAAAUGGCUGUG |
| | | | | C |
| siRNA 1402 | 1402 | CACAGCCAUUUUCCUUAAA | 7756 | UUUAAGGAAAAUGGCUGU |
| | | | | G |
| siRNA 1403 | 1403 | ACAGCCAUUUUCCUUAAAG | 7757 | CUUUAAGGAAAAUGGCUG |
| | | | | U |
| siRNA 1404 | 1404 | CAGCCAUUUUCCUUAAAGG | 7758 | CCUUUAAGGAAAAUGGCU |
| | | | | G |
| siRNA 1405 | 1405 | AGCCAUUUUCCUUAAAGGA | 7759 | UCCUUUAAGGAAAAUGGC |
| | | | | U |
| siRNA 1406 | 1406 | GCCAUUUUCCUUAAAGGAA | 7760 | UUCCUUUAAGGAAAAUGG |
| | | | | C |
| siRNA 1407 | 1407 | CCAUUUUCCUUAAAGGAAU | 7761 | AUUCCUUUAAGGAAAAUG |
| | | | | G |
| siRNA 1408 | 1408 | CAUUUUCCUUAAAGGAAU | 7762 | UAUUCCUUUAAGGAAAAU |
| | | A | | G |
| siRNA 1409 | 1409 | AUUUUCCUUAAAGGAAUA | 7763 | AUAUUCCUUUAAGGAAAA |
| | | U | | U |
| siRNA 1410 | 1410 | UUUUCCUUAAAGGAAUAU | 7764 | AAUAUUCCUUUAAGGAAA |
| | | U | | A |
| siRNA 1411 | 1411 | UUUCCUUAAAGGAAUAUU | 7765 | AAAUAUUCCUUUAAGGAA |
| | | U | | A |
| siRNA 1412 | 1412 | UUCCUUAAAGGAAUAUUU | 7766 | UAAAUAUUCCUUUAAGGA |
| | | A | | A |
| siRNA 1413 | 1413 | UCCUUAAAGGAAUAUUUA | 7767 | CUAAAUAUUCCUUUAAGG |
| | | G | | A |
| siRNA 1414 | 1414 | CCUUAAAGGAAUAUUUAG | 7768 | UCUAAAUAUUCCUUUAAG |
| | | A | | G |
| siRNA 1415 | 1415 | CUUAAAGGAAUAUUUAGA | 7769 | UUCUAAAUAUUCCUUUAA |
| | | A | | G |
| siRNA 1416 | 1416 | UUAAAGGAAUAUUUAGAA | 7770 | UUUCUAAAUAUUCCUUUA |
| | | A | | A |
| siRNA 1417 | 1417 | UAAAGGAAUAUUUAGAAA | 7771 | CUUUCUAAAUAUUCCUUUA |
| | | G | | |
| siRNA 1418 | 1418 | AAAGGAAUAUUUAGAAAG | 7772 | GCUUUCUAAAUAUUCCUUU |
| | | C | | |
| siRNA 1419 | 1419 | AAGGAAUAUUUAGAAAGC | 7773 | GGCUUUCUAAAUAUUCCUU |
| | | C | | |
| siRNA 1420 | 1420 | AGGAAUAUUUAGAAAGCC | 7774 | UGGCUUUCUAAAUAUUCCU |
| | | A | | |
| siRNA 1421 | 1421 | GGAAUAUUUAGAAAGCCA | 7775 | UUGGCUUUCUAAAUAUUCC |
| | | A | | |
| siRNA 1422 | 1422 | GAAUAUUUAGAAAGCCAA | 7776 | UUUGGCUUUCUAAAUAUU |
| | | A | | C |
| siRNA 1423 | 1423 | AAUAUUUAGAAAGCCAAA | 7777 | CUUUGGCUUUCUAAAUAU |
| | | G | | U |
| siRNA 1424 | 1424 | AUAUUUAGAAAGCCAAAG | 7778 | ACUUUGGCUUUCUAAAUA |
| | | U | | U |
| siRNA 1425 | 1425 | UAUUUAGAAAGCCAAAGU | 7779 | GACUUUGGCUUUCUAAAU |
| | | C | | A |
| siRNA 1426 | 1426 | AUUUAGAAAGCCAAAGUC | 7780 | UGACUUUGGCUUUCUAAA |
| | | A | | U |
| siRNA 1427 | 1427 | UUUAGAAAGCCAAAGUCA | 7781 | CUGACUUUGGCUUUCUAAA |
| | | G | | |
| siRNA 1428 | 1428 | UUAGAAAGCCAAAGUCAG | 7782 | UCUGACUUUGGCUUUCUAA |
| | | A | | |
| siRNA 1429 | 1429 | UAGAAAGCCAAAGUCAGA | 7783 | UUCUGACUUUGGCUUUCUA |
| | | A | | |
| siRNA 1430 | 1430 | AGAAAGCCAAAGUCAGAA | 7784 | UUUCUGACUUUGGCUUUCU |
| | | A | | |
| siRNA 1431 | 1431 | GAAAGCCAAAGUCAGAAAC | 7785 | GUUUCUGACUUUGGCUUUC |
| siRNA 1432 | 1432 | AAAGCCAAAGUCAGAAACC | 7786 | GGUUUCUGACUUUGGCUU |
| | | | | U |
| siRNA 1433 | 1433 | AAGCCAAAGUCAGAAACCG | 7787 | CGGUUUCUGACUUUGGCUU |
| siRNA 1434 | 1434 | AGCCAAAGUCAGAAACCGG | 7788 | CCGGUUUCUGACUUUGGCU |
| siRNA 1435 | 1435 | GCCAAAGUCAGAAACCGGU | 7789 | ACCGGUUUCUGACUUUGGC |
| siRNA 1436 | 1436 | CCAAAGUCAGAAACCGGUG | 7790 | CACCGGUUUCUGACUUUGG |
| siRNA 1437 | 1437 | CAAAGUCAGAAACCGGUGU | 7791 | ACACCGGUUUCUGACUUUG |
| siRNA 1438 | 1438 | AAAGUCAGAAACCGGUGUC | 7792 | GACACCGGUUUCUGACUUU |
| siRNA 1439 | 1439 | AAGUCAGAAACCGGUGUCU | 7793 | AGACACCGGUUUCUGACUU |
| siRNA 1440 | 1440 | AGUCAGAAACCGGUGUCUG | 7794 | CAGACACCGGUUUCUGACU |
| siRNA 1441 | 1441 | GUCAGAAACCGGUGUCUGC | 7795 | GCAGACACCGGUUUCUGAC |
| siRNA 1442 | 1442 | UCAGAAACCGGUGUCUGCU | 7796 | AGCAGACACCGGUUUCUGA |
| siRNA 1443 | 1443 | CAGAAACCGGUGUCUGCUC | 7797 | GAGCAGACACCGGUUUCUG |
| siRNA 1444 | 1444 | AGAAACCGGUGUCUGCUCU | 7798 | AGAGCAGACACCGGUUUCU |
| siRNA 1445 | 1445 | GAAACCGGUGUCUGCUCUA | 7799 | UAGAGCAGACACCGGUUUC |
| siRNA 1446 | 1446 | AAACCGGUGUCUGCUCUAC | 7800 | GUAGAGCAGACACCGGUUU |
| siRNA 1447 | 1447 | AACCGGUGUCUGCUCUACU | 7801 | AGUAGAGCAGACACCGGUU |
| siRNA 1448 | 1448 | ACCGGUGUCUGCUCUACUU | 7802 | AAGUAGAGCAGACACCGGU |
| siRNA 1449 | 1449 | CCGGUGUCUGCUCUACUUU | 7803 | AAAGUAGAGCAGACACCGG |
| siRNA 1450 | 1450 | CGGUGUCUGCUCUACUUUC | 7804 | GAAAGUAGAGCAGACACCG |
| siRNA 1451 | 1451 | GGUGUCUGCUCUACUUUCC | 7805 | GGAAAGUAGAGCAGACACC |
| siRNA 1452 | 1452 | GUGUCUGCUCUACUUUCCC | 7806 | GGGAAAGUAGAGCAGACA |
| | | | | C |
| siRNA 1453 | 1453 | UGUCUGCUCUACUUUCCCU | 7807 | AGGGAAAGUAGAGCAGAC |
| | | | | A |
| siRNA 1454 | 1454 | GUCUGCUCUACUUUCCCUG | 7808 | CAGGGAAAGUAGAGCAGA |
| | | | | C |
| siRNA 1455 | 1455 | UCUGCUCUACUUUCCCUGG | 7809 | CCAGGGAAAGUAGAGCAG |
| | | | | A |
| siRNA 1456 | 1456 | CUGCUCUACUUUCCCUGGA | 7810 | UCCAGGGAAAGUAGAGCA |
| | | | | G |
| siRNA 1457 | 1457 | UGCUCUACUUUCCCUGGAG | 7811 | CUCCAGGGAAAGUAGAGCA |
| siRNA 1458 | 1458 | GCUCUACUUUCCCUGGAGC | 7812 | GCUCCAGGGAAAGUAGAGC |
| siRNA 1459 | 1459 | CUCUACUUUCCCUGGAGCA | 7813 | UGCUCCAGGGAAAGUAGA |
| | | | | G |
| siRNA 1460 | 1460 | UCUACUUUCCCUGGAGCAA | 7814 | UUGCUCCAGGGAAAGUAG |
| | | | | A |
| siRNA 1461 | 1461 | CUACUUUCCCUGGAGCAAG | 7815 | CUUGCUCCAGGGAAAGUAG |
| siRNA 1462 | 1462 | UACUUUCCCUGGAGCAAGC | 7816 | GCUUGCUCCAGGGAAAGUA |
| siRNA 1463 | 1463 | ACUUUCCCUGGAGCAAGCG | 7817 | CGCUUGCUCCAGGGAAAGU |
| siRNA 1464 | 1464 | CUUUCCCUGGAGCAAGCGU | 7818 | ACGCUUGCUCCAGGGAAAG |
| siRNA 1465 | 1465 | UUUCCCUGGAGCAAGCGUU | 7819 | AACGCUUGCUCCAGGGAAA |
| siRNA 1466 | 1466 | UUCCCUGGAGCAAGCGUUG | 7820 | CAACGCUUGCUCCAGGGAA |
| siRNA 1467 | 1467 | UCCCUGGAGCAAGCGUUGU | 7821 | ACAACGCUUGCUCCAGGGA |
| siRNA 1468 | 1468 | CCCUGGAGCAAGCGUUGUU | 7822 | AACAACGCUUGCUCCAGGG |
| siRNA 1469 | 1469 | CCUGGAGCAAGCGUUGUUA | 7823 | UAACAACGCUUGCUCCAGG |
| siRNA 1470 | 1470 | CUGGAGCAAGCGUUGUUAC | 7824 | GUAACAACGCUUGCUCCAG |
| siRNA 1471 | 1471 | UGGAGCAAGCGUUGUUACC | 7825 | GGUAACAACGCUUGCUCCA |
| siRNA 1472 | 1472 | GGAGCAAGCGUUGUUACCA | 7826 | UGGUAACAACGCUUGCUCC |
| siRNA 1473 | 1473 | GAGCAAGCGUUGUUACCAG | 7827 | CUGGUAACAACGCUUGCUC |
| siRNA 1474 | 1474 | AGCAAGCGUUGUUACCAGC | 7828 | GCUGGUAACAACGCUUGCU |
| siRNA 1475 | 1475 | GCAAGCGUUGUUACCAGCU | 7829 | AGCUGGUAACAACGCUUGC |
| siRNA 1476 | 1476 | CAAGCGUUGUUACCAGCUA | 7830 | UAGCUGGUAACAACGCUUG |
| siRNA 1477 | 1477 | AAGCGUUGUUACCAGCUAU | 7831 | AUAGCUGGUAACAACGCUU |
| siRNA 1478 | 1478 | AGCGUUGUUACCAGCUAUA | 7832 | UAUAGCUGGUAACAACGCU |
| siRNA 1479 | 1479 | GCGUUGUUACCAGCUAUAC | 7833 | GUAUAGCUGGUAACAACGC |
| siRNA 1480 | 1480 | CGUUGUUACCAGCUAUACU | 7834 | AGUAUAGCUGGUAACAAC |
| | | | | G |
| siRNA 1481 | 1481 | GUUGUUACCAGCUAUACUU | 7835 | AAGUAUAGCUGGUAACAA |
| | | | | C |
| siRNA 1482 | 1482 | UUGUUACCAGCUAUACUUC | 7836 | GAAGUAUAGCUGGUAACA |
| | | | | A |
| siRNA 1483 | 1483 | UGUUACCAGCUAUACUUCC | 7837 | GGAAGUAUAGCUGGUAAC |
| | | | | A |
| siRNA 1484 | 1484 | GUUACCAGCUAUACUUCCU | 7838 | AGGAAGUAUAGCUGGUAA |
| | | | | C |
| siRNA 1485 | 1485 | UUACCAGCUAUACUUCCUU | 7839 | AAGGAAGUAUAGCUGGUA |
| | | | | A |
| siRNA 1486 | 1486 | UACCAGCUAUACUUCCUUC | 7840 | GAAGGAAGUAUAGCUGGU |
| | | | | A |
| siRNA 1487 | 1487 | ACCAGCUAUACUUCCUUCA | 7841 | UGAAGGAAGUAUAGCUGG |
| | | | | U |
| siRNA 1488 | 1488 | CCAGCUAUACUUCCUUCAA | 7842 | UUGAAGGAAGUAUAGCUG |
| | | | | G |
| siRNA 1489 | 1489 | CAGCUAUACUUCCUUCAAG | 7843 | CUUGAAGGAAGUAUAGCU |
| | | | | G |
| siRNA 1490 | 1490 | AGCUAUACUUCCUUCAAGA | 7844 | UCUUGAAGGAAGUAUAGC |
| | | | | U |
| siRNA 1491 | 1491 | GCUAUACUUCCUUCAAGAC | 7845 | GUCUUGAAGGAAGUAUAG |
| | | | | C |
| siRNA 1492 | 1492 | CUAUACUUCCUUCAAGACC | 7846 | GGUCUUGAAGGAAGUAUA |
| | | | | G |
| siRNA 1493 | 1493 | UAUACUUCCUUCAAGACCC | 7847 | GGGUCUUGAAGGAAGUAU |
| | | | | A |
| siRNA 1494 | 1494 | AUACUUCCUUCAAGACCCA | 7848 | UGGGUCUUGAAGGAAGUA |
| | | | | U |
| siRNA 1495 | 1495 | UACUUCCUUCAAGACCCAG | 7849 | CUGGGUCUUGAAGGAAGU |
| | | | | A |
| siRNA 1496 | 1496 | ACUUCCUUCAAGACCCAGU | 7850 | ACUGGGUCUUGAAGGAAG |
| | | | | U |
| siRNA 1497 | 1497 | CUUCCUUCAAGACCCAGUG | 7851 | CACUGGGUCUUGAAGGAA |
| | | | | G |
| siRNA 1498 | 1498 | UUCCUUCAAGACCCAGUGA | 7852 | UCACUGGGUCUUGAAGGA |
| | | | | A |
| siRNA 1499 | 1499 | UCCUUCAAGACCCAGUGAU | 7853 | AUCACUGGGUCUUGAAGG |
| | | | | A |
| siRNA 1500 | 1500 | CCUUCAAGACCCAGUGAUG | 7854 | CAUCACUGGGUCUUGAAGG |
| siRNA 1501 | 1501 | CUUCAAGACCCAGUGAUGC | 7855 | GCAUCACUGGGUCUUGAAG |
| siRNA 1502 | 1502 | UUCAAGACCCAGUGAUGCU | 7856 | AGCAUCACUGGGUCUUGAA |
| siRNA 1503 | 1503 | UCAAGACCCAGUGAUGCUG | 7857 | CAGCAUCACUGGGUCUUGA |
| siRNA 1504 | 1504 | CAAGACCCAGUGAUGCUGC | 7858 | GCAGCAUCACUGGGUCUUG |
| siRNA 1505 | 1505 | AAGACCCAGUGAUGCUGCU | 7859 | AGCAGCAUCACUGGGUCUU |
| siRNA 1506 | 1506 | AGACCCAGUGAUGCUGCUG | 7860 | CAGCAGCAUCACUGGGUCU |
| siRNA 1507 | 1507 | GACCCAGUGAUGCUGCUGA | 7861 | UCAGCAGCAUCACUGGGUC |
| siRNA 1508 | 1508 | ACCCAGUGAUGCUGCUGAU | 7862 | AUCAGCAGCAUCACUGGGU |
| siRNA 1509 | 1509 | CCCAGUGAUGCUGCUGAUG | 7863 | CAUCAGCAGCAUCACUGGG |
| siRNA 1510 | 1510 | CCAGUGAUGCUGCUGAUGA | 7864 | UCAUCAGCAGCAUCACUGG |
| siRNA 1511 | 1511 | CAGUGAUGCUGCUGAUGA | 7865 | UUCAUCAGCAGCAUCACUG |
| | | A | | |
| siRNA 1512 | 1512 | AGUGAUGCUGCUGAUGAA | 7866 | CUUCAUCAGCAGCAUCACU |
| | | G | | |
| siRNA 1513 | 1513 | GUGAUGCUGCUGAUGAAG | 7867 | CCUUCAUCAGCAGCAUCAC |
| | | G | | |
| siRNA 1514 | 1514 | UGAUGCUGCUGAUGAAGG | 7868 | ACCUUCAUCAGCAGCAUCA |
| | | U | | |
| siRNA 1515 | 1515 | GAUGCUGCUGAUGAAGGU | 7869 | UACCUUCAUCAGCAGCAUC |
| | | A | | |
| siRNA 1516 | 1516 | AUGCUGCUGAUGAAGGUA | 7870 | CUACCUUCAUCAGCAGCAU |
| | | G | | |
| siRNA 1517 | 1517 | UGCUGCUGAUGAAGGUAG | 7871 | UCUACCUUCAUCAGCAGCA |
| | | A | | |
| siRNA 1518 | 1518 | GCUGCUGAUGAAGGUAGA | 7872 | CUCUACCUUCAUCAGCAGC |
| | | G | | |
| siRNA 1519 | 1519 | CUGCUGAUGAAGGUAGAG | 7873 | UCUCUACCUUCAUCAGCAG |
| | | A | | |
| siRNA 1520 | 1520 | UGCUGAUGAAGGUAGAGA | 7874 | GUCUCUACCUUCAUCAGCA |
| | | C | | |
| siRNA 1521 | 1521 | GCUGAUGAAGGUAGAGAC | 7875 | UGUCUCUACCUUCAUCAGC |
| | | A | | |
| siRNA 1522 | 1522 | CUGAUGAAGGUAGAGACA | 7876 | GUGUCUCUACCUUCAUCAG |
| | | C | | |
| siRNA 1523 | 1523 | UGAUGAAGGUAGAGACAC | 7877 | CGUGUCUCUACCUUCAUCA |
| | | G | | |
| siRNA 1524 | 1524 | GAUGAAGGUAGAGACACG | 7878 | ACGUGUCUCUACCUUCAUC |
| | | U | | |
| siRNA 1525 | 1525 | AUGAAGGUAGAGACACGU | 7879 | GACGUGUCUCUACCUUCAU |
| | | C | | |
| siRNA 1526 | 1526 | UGAAGGUAGAGACACGUCC | 7880 | GGACGUGUCUCUACCUUCA |
| siRNA 1527 | 1527 | GAAGGUAGAGACACGUCCA | 7881 | UGGACGUGUCUCUACCUUC |
| siRNA 1528 | 1528 | AAGGUAGAGACACGUCCAU | 7882 | AUGGACGUGUCUCUACCUU |
| siRNA 1529 | 1529 | AGGUAGAGACACGUCCAUU | 7883 | AAUGGACGUGUCUCUACCU |
| siRNA 1530 | 1530 | GGUAGAGACACGUCCAUUA | 7884 | UAAUGGACGUGUCUCUACC |
| siRNA 1531 | 1531 | GUAGAGACACGUCCAUUAA | 7885 | UUAAUGGACGUGUCUCUAC |
| siRNA 1532 | 1532 | UAGAGACACGUCCAUUAAU | 7886 | AUUAAUGGACGUGUCUCU |
| | | | | A |
| siRNA 1533 | 1533 | AGAGACACGUCCAUUAAUG | 7887 | CAUUAAUGGACGUGUCUCU |
| siRNA 1534 | 1534 | GAGACACGUCCAUUAAUGA | 7888 | UCAUUAAUGGACGUGUCUC |
| siRNA 1535 | 1535 | AGACACGUCCAUUAAUGAG | 7889 | CUCAUUAAUGGACGUGUCU |
| siRNA 1536 | 1536 | GACACGUCCAUUAAUGAGU | 7890 | ACUCAUUAAUGGACGUGUC |
| siRNA 1537 | 1537 | ACACGUCCAUUAAUGAGUC | 7891 | GACUCAUUAAUGGACGUG |
| | | | | U |
| siRNA 1538 | 1538 | CACGUCCAUUAAUGAGUCC | 7892 | GGACUCAUUAAUGGACGU |
| | | | | G |
| siRNA 1539 | 1539 | ACGUCCAUUAAUGAGUCCA | 7893 | UGGACUCAUUAAUGGACG |
| | | | | U |
| siRNA 1540 | 1540 | CGUCCAUUAAUGAGUCCAG | 7894 | CUGGACUCAUUAAUGGACG |
| siRNA 1541 | 1541 | GUCCAUUAAUGAGUCCAGA | 7895 | UCUGGACUCAUUAAUGGAC |
| siRNA 1542 | 1542 | UCCAUUAAUGAGUCCAGAA | 7896 | UUCUGGACUCAUUAAUGG |
| | | | | A |
| siRNA 1543 | 1543 | CCAUUAAUGAGUCCAGAAA | 7897 | UUUCUGGACUCAUUAAUG |
| | | | | G |
| siRNA 1544 | 1544 | CAUUAAUGAGUCCAGAAA | 7898 | AUUUCUGGACUCAUUAAU |
| | | U | | G |
| siRNA 1545 | 1545 | AUUAAUGAGUCCAGAAAU | 7899 | CAUUUCUGGACUCAUUAAU |
| | | G | | |
| siRNA 1546 | 1546 | UUAAUGAGUCCAGAAAUG | 7900 | GCAUUUCUGGACUCAUUAA |
| | | C | | |
| siRNA 1547 | 1547 | UAAUGAGUCCAGAAAUGC | 7901 | UGCAUUUCUGGACUCAUUA |
| | | A | | |
| siRNA 1548 | 1548 | AAUGAGUCCAGAAAUGCA | 7902 | UUGCAUUUCUGGACUCAUU |
| | | A | | |
| siRNA 1549 | 1549 | AUGAGUCCAGAAAUGCAAC | 7903 | GUUGCAUUUCUGGACUCAU |
| siRNA 1550 | 1550 | UGAGUCCAGAAAUGCAACA | 7904 | UGUUGCAUUUCUGGACUCA |
| siRNA 1551 | 1551 | GAGUCCAGAAAUGCAACAG | 7905 | CUGUUGCAUUUCUGGACUC |
| siRNA 1552 | 1552 | AGUCCAGAAAUGCAACAGA | 7906 | UCUGUUGCAUUUCUGGACU |
| siRNA 1553 | 1553 | GUCCAGAAAUGCAACAGAU | 7907 | AUCUGUUGCAUUUCUGGAC |
| siRNA 1554 | 1554 | UCCAGAAAUGCAACAGAUG | 7908 | CAUCUGUUGCAUUUCUGGA |
| siRNA 1555 | 1555 | CCAGAAAUGCAACAGAUGA | 7909 | UCAUCUGUUGCAUUUCUGG |
| siRNA 1556 | 1556 | CAGAAAUGCAACAGAUGA | 7910 | UUCAUCUGUUGCAUUUCUG |
| | | A | | |
| siRNA 1557 | 1557 | AGAAAUGCAACAGAUGAA | 7911 | AUUCAUCUGUUGCAUUUCU |
| | | U | | |
| siRNA 1558 | 1558 | GAAAUGCAACAGAUGAAU | 7912 | GAUUCAUCUGUUGCAUUUC |
| | | C | | |
| siRNA 1559 | 1559 | AAAUGCAACAGAUGAAUCC | 7913 | GGAUUCAUCUGUUGCAUU |
| | | | | U |
| siRNA 1560 | 1560 | AAUGCAACAGAUGAAUCCC | 7914 | GGGAUUCAUCUGUUGCAU |
| | | | | U |
| siRNA 1561 | 1561 | AUGCAACAGAUGAAUCCCU | 7915 | AGGGAUUCAUCUGUUGCA |
| | | | | U |
| siRNA 1562 | 1562 | UGCAACAGAUGAAUCCCUA | 7916 | UAGGGAUUCAUCUGUUGC |
| | | | | A |
| siRNA 1563 | 1563 | GCAACAGAUGAAUCCCUAC | 7917 | GUAGGGAUUCAUCUGUUG |
| | | | | C |
| siRNA 1564 | 1564 | CAACAGAUGAAUCCCUACG | 7918 | CGUAGGGAUUCAUCUGUU |
| | | | | G |
| siRNA 1565 | 1565 | AACAGAUGAAUCCCUACGA | 7919 | UCGUAGGGAUUCAUCUGU |
| | | | | U |
| siRNA 1566 | 1566 | ACAGAUGAAUCCCUACGAA | 7920 | UUCGUAGGGAUUCAUCUG |
| | | | | U |
| siRNA 1567 | 1567 | CAGAUGAAUCCCUACGAAG | 7921 | CUUCGUAGGGAUUCAUCUG |
| siRNA 1568 | 1568 | AGAUGAAUCCCUACGAAGG | 7922 | CCUUCGUAGGGAUUCAUCU |
| siRNA 1569 | 1569 | GAUGAAUCCCUACGAAGGA | 7923 | UCCUUCGUAGGGAUUCAUC |
| siRNA 1570 | 1570 | AUGAAUCCCUACGAAGGAG | 7924 | CUCCUUCGUAGGGAUUCAU |
| siRNA 1571 | 1571 | UGAAUCCCUACGAAGGAGG | 7925 | CCUCCUUCGUAGGGAUUCA |
| siRNA 1572 | 1572 | GAAUCCCUACGAAGGAGGU | 7926 | ACCUCCUUCGUAGGGAUUC |
| siRNA 1573 | 1573 | AAUCCCUACGAAGGAGGUU | 7927 | AACCUCCUUCGUAGGGAUU |
| siRNA 1574 | 1574 | AUCCCUACGAAGGAGGUUG | 7928 | CAACCUCCUUCGUAGGGAU |
| siRNA 1575 | 1575 | UCCCUACGAAGGAGGUUGA | 7929 | UCAACCUCCUUCGUAGGGA |
| siRNA 1576 | 1576 | CCCUACGAAGGAGGUUGAU | 7930 | AUCAACCUCCUUCGUAGGG |
| siRNA 1577 | 1577 | CCUACGAAGGAGGUUGAU | 7931 | AAUCAACCUCCUUCGUAGG |
| | | U | | |
| siRNA 1578 | 1578 | CUACGAAGGAGGUUGAUU | 7932 | CAAUCAACCUCCUUCGUAG |
| | | G | | |
| siRNA 1579 | 1579 | UACGAAGGAGGUUGAUUG | 7933 | GCAAUCAACCUCCUUCGUA |
| | | C | | |
| siRNA 1580 | 1580 | ACGAAGGAGGUUGAUUGC | 7934 | UGCAAUCAACCUCCUUCGU |
| | | A | | |
| siRNA 1581 | 1581 | CGAAGGAGGUUGAUUGCA | 7935 | UUGCAAUCAACCUCCUUCG |
| | | A | | |
| siRNA 1582 | 1582 | GAAGGAGGUUGAUUGCAA | 7936 | UUUGCAAUCAACCUCCUUC |
| | | A | | |
| siRNA 1583 | 1583 | AAGGAGGUUGAUUGCAAA | 7937 | AUUUGCAAUCAACCUCCUU |
| | | U | | |
| siRNA 1584 | 1584 | AGGAGGUUGAUUGCAAAU | 7938 | GAUUUGCAAUCAACCUCCU |
| | | C | | |
| siRNA 1585 | 1585 | GGAGGUUGAUUGCAAAUC | 7939 | AGAUUUGCAAUCAACCUCC |
| | | U | | |
| siRNA 1586 | 1586 | GAGGUUGAUUGCAAAUCU | 7940 | CAGAUUUGCAAUCAACCUC |
| | | G | | |
| siRNA 1587 | 1587 | AGGUUGAUUGCAAAUCUG | 7941 | CCAGAUUUGCAAUCAACCU |
| | | G | | |
| siRNA 1588 | 1588 | GGUUGAUUGCAAAUCUGG | 7942 | GCCAGAUUUGCAAUCAACC |
| | | C | | |
| siRNA 1589 | 1589 | GUUGAUUGCAAAUCUGGC | 7943 | AGCCAGAUUUGCAAUCAAC |
| | | U | | |
| siRNA 1590 | 1590 | UUGAUUGCAAAUCUGGCU | 7944 | CAGCCAGAUUUGCAAUCAA |
| | | G | | |
| siRNA 1591 | 1591 | UGAUUGCAAAUCUGGCUG | 7945 | UCAGCCAGAUUUGCAAUCA |
| | | A | | |
| siRNA 1592 | 1592 | GAUUGCAAAUCUGGCUGA | 7946 | CUCAGCCAGAUUUGCAAUC |
| | | G | | |
| siRNA 1593 | 1593 | AUUGCAAAUCUGGCUGAGC | 7947 | GCUCAGCCAGAUUUGCAAU |
| siRNA 1594 | 1594 | UUGCAAAUCUGGCUGAGCA | 7948 | UGCUCAGCCAGAUUUGCAA |
| siRNA 1595 | 1595 | UGCAAAUCUGGCUGAGCAU | 7949 | AUGCUCAGCCAGAUUUGCA |
| siRNA 1596 | 1596 | GCAAAUCUGGCUGAGCAUA | 7950 | UAUGCUCAGCCAGAUUUGC |
| siRNA 1597 | 1597 | CAAAUCUGGCUGAGCAUAU | 7951 | AUAUGCUCAGCCAGAUUUG |
| siRNA 1598 | 1598 | AAAUCUGGCUGAGCAUAU | 7952 | AAUAUGCUCAGCCAGAUUU |
| | | U | | |
| siRNA 1599 | 1599 | AAUCUGGCUGAGCAUAUUC | 7953 | GAAUAUGCUCAGCCAGAUU |
| siRNA 1600 | 1600 | AUCUGGCUGAGCAUAUUCU | 7954 | AGAAUAUGCUCAGCCAGAU |
| siRNA 1601 | 1601 | UCUGGCUGAGCAUAUUCUA | 7955 | UAGAAUAUGCUCAGCCAGA |
| siRNA 1602 | 1602 | CUGGCUGAGCAUAUUCUAU | 7956 | AUAGAAUAUGCUCAGCCAG |
| siRNA 1603 | 1603 | UGGCUGAGCAUAUUCUAU | 7957 | AAUAGAAUAUGCUCAGCCA |
| | | U | | |
| siRNA 1604 | 1604 | GGCUGAGCAUAUUCUAUUC | 7958 | GAAUAGAAUAUGCUCAGCC |
| siRNA 1605 | 1605 | GCUGAGCAUAUUCUAUUCA | 7959 | UGAAUAGAAUAUGCUCAG |
| | | | | C |
| siRNA 1606 | 1606 | CUGAGCAUAUUCUAUUCAC | 7960 | GUGAAUAGAAUAUGCUCA |
| | | | | G |
| siRNA 1607 | 1607 | UGAGCAUAUUCUAUUCACU | 7961 | AGUGAAUAGAAUAUGCUC |
| | | | | A |
| siRNA 1608 | 1608 | GAGCAUAUUCUAUUCACUG | 7962 | CAGUGAAUAGAAUAUGCU |
| | | | | C |
| siRNA 1609 | 1609 | AGCAUAUUCUAUUCACUGC | 7963 | GCAGUGAAUAGAAUAUGC |
| | | | | U |
| siRNA 1610 | 1610 | GCAUAUUCUAUUCACUGCU | 7964 | AGCAGUGAAUAGAAUAUG |
| | | | | C |
| siRNA 1611 | 1611 | CAUAUUCUAUUCACUGCUA | 7965 | UAGCAGUGAAUAGAAUAU |
| | | | | G |
| siRNA 1612 | 1612 | AUAUUCUAUUCACUGCUAG | 7966 | CUAGCAGUGAAUAGAAUA |
| | | | | U |
| siRNA 1613 | 1613 | UAUUCUAUUCACUGCUAGC | 7967 | GCUAGCAGUGAAUAGAAU |
| | | | | A |
| siRNA 1614 | 1614 | AUUCUAUUCACUGCUAGCA | 7968 | UGCUAGCAGUGAAUAGAA |
| | | | | U |
| siRNA 1615 | 1615 | UUCUAUUCACUGCUAGCAA | 7969 | UUGCUAGCAGUGAAUAGA |
| | | | | A |
| siRNA 1616 | 1616 | UCUAUUCACUGCUAGCAAG | 7970 | CUUGCUAGCAGUGAAUAG |
| | | | | A |
| siRNA 1617 | 1617 | CUAUUCACUGCUAGCAAGU | 7971 | ACUUGCUAGCAGUGAAUA |
| | | | | G |
| siRNA 1618 | 1618 | UAUUCACUGCUAGCAAGUC | 7972 | GACUUGCUAGCAGUGAAU |
| | | | | A |
| siRNA 1619 | 1619 | AUUCACUGCUAGCAAGUCC | 7973 | GGACUUGCUAGCAGUGAA |
| | | | | U |
| siRNA 1620 | 1620 | UUCACUGCUAGCAAGUCCU | 7974 | AGGACUUGCUAGCAGUGA |
| | | | | A |
| siRNA 1621 | 1621 | UCACUGCUAGCAAGUCCUG | 7975 | CAGGACUUGCUAGCAGUGA |
| siRNA 1622 | 1622 | CACUGCUAGCAAGUCCUGU | 7976 | ACAGGACUUGCUAGCAGUG |
| siRNA 1623 | 1623 | ACUGCUAGCAAGUCCUGUG | 7977 | CACAGGACUUGCUAGCAGU |
| siRNA 1624 | 1624 | CUGCUAGCAAGUCCUGUGC | 7978 | GCACAGGACUUGCUAGCAG |
| siRNA 1625 | 1625 | UGCUAGCAAGUCCUGUGCC | 7979 | GGCACAGGACUUGCUAGCA |
| siRNA 1626 | 1626 | GCUAGCAAGUCCUGUGCCA | 7980 | UGGCACAGGACUUGCUAGC |
| siRNA 1627 | 1627 | CUAGCAAGUCCUGUGCCAU | 7981 | AUGGCACAGGACUUGCUAG |
| siRNA 1628 | 1628 | UAGCAAGUCCUGUGCCAUU | 7982 | AAUGGCACAGGACUUGCUA |
| siRNA 1629 | 1629 | AGCAAGUCCUGUGCCAUUA | 7983 | UAAUGGCACAGGACUUGCU |
| siRNA 1630 | 1630 | GCAAGUCCUGUGCCAUUAU | 7984 | AUAAUGGCACAGGACUUGC |
| siRNA 1631 | 1631 | CAAGUCCUGUGCCAUUAUG | 7985 | CAUAAUGGCACAGGACUUG |
| siRNA 1632 | 1632 | AAGUCCUGUGCCAUUAUGU | 7986 | ACAUAAUGGCACAGGACUU |
| siRNA 1633 | 1633 | AGUCCUGUGCCAUUAUGUC | 7987 | GACAUAAUGGCACAGGACU |
| siRNA 1634 | 1634 | GUCCUGUGCCAUUAUGUCC | 7988 | GGACAUAAUGGCACAGGAC |
| siRNA 1635 | 1635 | UCCUGUGCCAUUAUGUCCA | 7989 | UGGACAUAAUGGCACAGG |
| | | | | A |
| siRNA 1636 | 1636 | CCUGUGCCAUUAUGUCCAC | 7990 | GUGGACAUAAUGGCACAG |
| | | | | G |
| siRNA 1637 | 1637 | CUGUGCCAUUAUGUCCACA | 7991 | UGUGGACAUAAUGGCACA |
| | | | | G |
| siRNA 1638 | 1638 | UGUGCCAUUAUGUCCACAC | 7992 | GUGUGGACAUAAUGGCAC |
| | | | | A |
| siRNA 1639 | 1639 | GUGCCAUUAUGUCCACACA | 7993 | UGUGUGGACAUAAUGGCA |
| | | | | C |
| siRNA 1640 | 1640 | UGCCAUUAUGUCCACACAC | 7994 | GUGUGUGGACAUAAUGGC |
| | | | | A |
| siRNA 1641 | 1641 | GCCAUUAUGUCCACACACA | 7995 | UGUGUGUGGACAUAAUGG |
| | | | | C |
| siRNA 1642 | 1642 | CCAUUAUGUCCACACACAU | 7996 | AUGUGUGUGGACAUAAUG |
| | | | | G |
| siRNA 1643 | 1643 | CAUUAUGUCCACACACAUU | 7997 | AAUGUGUGUGGACAUAAU |
| | | | | G |
| siRNA 1644 | 1644 | AUUAUGUCCACACACAUUG | 7998 | CAAUGUGUGUGGACAUAA |
| | | | | U |
| siRNA 1645 | 1645 | UUAUGUCCACACACAUUGU | 7999 | ACAAUGUGUGUGGACAUA |
| | | | | A |
| siRNA 1646 | 1646 | UAUGUCCACACACAUUGUG | 8000 | CACAAUGUGUGUGGACAU |
| | | | | A |
| siRNA 1647 | 1647 | AUGUCCACACACAUUGUGG | 8001 | CCACAAUGUGUGUGGACAU |
| siRNA 1648 | 1648 | UGUCCACACACAUUGUGGC | 8002 | GCCACAAUGUGUGUGGACA |
| siRNA 1649 | 1649 | GUCCACACACAUUGUGGCU | 8003 | AGCCACAAUGUGUGUGGAC |
| siRNA 1650 | 1650 | UCCACACACAUUGUGGCUU | 8004 | AAGCCACAAUGUGUGUGG |
| | | | | A |
| siRNA 1651 | 1651 | CCACACACAUUGUGGCUUG | 8005 | CAAGCCACAAUGUGUGUGG |
| siRNA 1652 | 1652 | CACACACAUUGUGGCUUGC | 8006 | GCAAGCCACAAUGUGUGUG |
| siRNA 1653 | 1653 | ACACACAUUGUGGCUUGCC | 8007 | GGCAAGCCACAAUGUGUGU |
| siRNA 1654 | 1654 | CACACAUUGUGGCUUGCCU | 8008 | AGGCAAGCCACAAUGUGUG |
| siRNA 1655 | 1655 | ACACAUUGUGGCUUGCCUG | 8009 | CAGGCAAGCCACAAUGUGU |
| siRNA 1656 | 1656 | CACAUUGUGGCUUGCCUGC | 8010 | GCAGGCAAGCCACAAUGUG |
| siRNA 1657 | 1657 | ACAUUGUGGCUUGCCUGCU | 8011 | AGCAGGCAAGCCACAAUGU |
| siRNA 1658 | 1658 | CAUUGUGGCUUGCCUGCUC | 8012 | GAGCAGGCAAGCCACAAUG |
| siRNA 1659 | 1659 | AUUGUGGCUUGCCUGCUCC | 8013 | GGAGCAGGCAAGCCACAAU |
| siRNA 1660 | 1660 | UUGUGGCUUGCCUGCUCCU | 8014 | AGGAGCAGGCAAGCCACAA |
| siRNA 1661 | 1661 | UGUGGCUUGCCUGCUCCUC | 8015 | GAGGAGCAGGCAAGCCACA |
| siRNA 1662 | 1662 | GUGGCUUGCCUGCUCCUCU | 8016 | AGAGGAGCAGGCAAGCCAC |
| siRNA 1663 | 1663 | UGGCUUGCCUGCUCCUCUA | 8017 | UAGAGGAGCAGGCAAGCCA |
| siRNA 1664 | 1664 | GGCUUGCCUGCUCCUCUAC | 8018 | GUAGAGGAGCAGGCAAGCC |
| siRNA 1665 | 1665 | GCUUGCCUGCUCCUCUACA | 8019 | UGUAGAGGAGCAGGCAAG |
| | | | | C |
| siRNA 1666 | 1666 | CUUGCCUGCUCCUCUACAG | 8020 | CUGUAGAGGAGCAGGCAA |
| | | | | G |
| siRNA 1667 | 1667 | UUGCCUGCUCCUCUACAGA | 8021 | UCUGUAGAGGAGCAGGCA |
| | | | | A |
| siRNA 1668 | 1668 | UGCCUGCUCCUCUACAGAC | 8022 | GUCUGUAGAGGAGCAGGC |
| | | | | A |
| siRNA 1669 | 1669 | GCCUGCUCCUCUACAGACA | 8023 | UGUCUGUAGAGGAGCAGG |
| | | | | C |
| siRNA 1670 | 1670 | CCUGCUCCUCUACAGACAC | 8024 | GUGUCUGUAGAGGAGCAG |
| | | | | G |
| siRNA 1671 | 1671 | CUGCUCCUCUACAGACACA | 8025 | UGUGUCUGUAGAGGAGCA |
| | | | | G |
| siRNA 1672 | 1672 | UGCUCCUCUACAGACACAG | 8026 | CUGUGUCUGUAGAGGAGC |
| | | | | A |
| siRNA 1673 | 1673 | GCUCCUCUACAGACACAGG | 8027 | CCUGUGUCUGUAGAGGAGC |
| siRNA 1674 | 1674 | CUCCUCUACAGACACAGGC | 8028 | GCCUGUGUCUGUAGAGGA |
| | | | | G |
| siRNA 1675 | 1675 | UCCUCUACAGACACAGGCA | 8029 | UGCCUGUGUCUGUAGAGG |
| | | | | A |
| siRNA 1676 | 1676 | CCUCUACAGACACAGGCAG | 8030 | CUGCCUGUGUCUGUAGAGG |
| siRNA 1677 | 1677 | CUCUACAGACACAGGCAGG | 8031 | CCUGCCUGUGUCUGUAGAG |
| siRNA 1678 | 1678 | UCUACAGACACAGGCAGGG | 8032 | CCCUGCCUGUGUCUGUAGA |
| siRNA 1679 | 1679 | CUACAGACACAGGCAGGGA | 8033 | UCCCUGCCUGUGUCUGUAG |
| siRNA 1680 | 1680 | UACAGACACAGGCAGGGAA | 8034 | UUCCCUGCCUGUGUCUGUA |
| siRNA 1681 | 1681 | ACAGACACAGGCAGGGAAU | 8035 | AUUCCCUGCCUGUGUCUGU |
| siRNA 1682 | 1682 | CAGACACAGGCAGGGAAUU | 8036 | AAUUCCCUGCCUGUGUCUG |
| siRNA 1683 | 1683 | AGACACAGGCAGGGAAUU | 8037 | CAAUUCCCUGCCUGUGUCU |
| | | G | | |
| siRNA 1684 | 1684 | GACACAGGCAGGGAAUUG | 8038 | UCAAUUCCCUGCCUGUGUC |
| | | A | | |
| siRNA 1685 | 1685 | ACACAGGCAGGGAAUUGA | 8039 | AUCAAUUCCCUGCCUGUGU |
| | | U | | |
| siRNA 1686 | 1686 | CACAGGCAGGGAAUUGAUC | 8040 | GAUCAAUUCCCUGCCUGUG |
| siRNA 1687 | 1687 | ACAGGCAGGGAAUUGAUC | 8041 | AGAUCAAUUCCCUGCCUGU |
| | | U | | |
| siRNA 1688 | 1688 | CAGGCAGGGAAUUGAUCUC | 8042 | GAGAUCAAUUCCCUGCCUG |
| siRNA 1689 | 1689 | AGGCAGGGAAUUGAUCUC | 8043 | AGAGAUCAAUUCCCUGCCU |
| | | U | | |
| siRNA 1690 | 1690 | GGCAGGGAAUUGAUCUCUC | 8044 | GAGAGAUCAAUUCCCUGCC |
| siRNA 1691 | 1691 | GCAGGGAAUUGAUCUCUCC | 8045 | GGAGAGAUCAAUUCCCUGC |
| siRNA 1692 | 1692 | CAGGGAAUUGAUCUCUCCA | 8046 | UGGAGAGAUCAAUUCCCUG |
| siRNA 1693 | 1693 | AGGGAAUUGAUCUCUCCAC | 8047 | GUGGAGAGAUCAAUUCCCU |
| siRNA 1694 | 1694 | GGGAAUUGAUCUCUCCACA | 8048 | UGUGGAGAGAUCAAUUCCC |
| siRNA 1695 | 1695 | GGAAUUGAUCUCUCCACAU | 8049 | AUGUGGAGAGAUCAAUUC |
| | | | | C |
| siRNA 1696 | 1696 | GAAUUGAUCUCUCCACAUU | 8050 | AAUGUGGAGAGAUCAAUU |
| | | | | C |
| siRNA 1697 | 1697 | AAUUGAUCUCUCCACAUUG | 8051 | CAAUGUGGAGAGAUCAAU |
| | | | | U |
| siRNA 1698 | 1698 | AUUGAUCUCUCCACAUUGG | 8052 | CCAAUGUGGAGAGAUCAA |
| | | | | U |
| siRNA 1699 | 1699 | UUGAUCUCUCCACAUUGGU | 8053 | ACCAAUGUGGAGAGAUCA |
| | | | | A |
| siRNA 1700 | 1700 | UGAUCUCUCCACAUUGGUC | 8054 | GACCAAUGUGGAGAGAUC |
| | | | | A |
| siRNA 1701 | 1701 | GAUCUCUCCACAUUGGUCG | 8055 | CGACCAAUGUGGAGAGAUC |
| siRNA 1702 | 1702 | AUCUCUCCACAUUGGUCGA | 8056 | UCGACCAAUGUGGAGAGA |
| | | | | U |
| siRNA 1703 | 1703 | UCUCUCCACAUUGGUCGAA | 8057 | UUCGACCAAUGUGGAGAG |
| | | | | A |
| siRNA 1704 | 1704 | CUCUCCACAUUGGUCGAAG | 8058 | CUUCGACCAAUGUGGAGAG |
| siRNA 1705 | 1705 | UCUCCACAUUGGUCGAAGA | 8059 | UCUUCGACCAAUGUGGAGA |
| siRNA 1706 | 1706 | CUCCACAUUGGUCGAAGAC | 8060 | GUCUUCGACCAAUGUGGAG |
| siRNA 1707 | 1707 | UCCACAUUGGUCGAAGACU | 8061 | AGUCUUCGACCAAUGUGGA |
| siRNA 1708 | 1708 | CCACAUUGGUCGAAGACUU | 8062 | AAGUCUUCGACCAAUGUGG |
| siRNA 1709 | 1709 | CACAUUGGUCGAAGACUUC | 8063 | GAAGUCUUCGACCAAUGUG |
| siRNA 1710 | 1710 | ACAUUGGUCGAAGACUUCU | 8064 | AGAAGUCUUCGACCAAUGU |
| siRNA 1711 | 1711 | CAUUGGUCGAAGACUUCUU | 8065 | AAGAAGUCUUCGACCAAUG |
| siRNA 1712 | 1712 | AUUGGUCGAAGACUUCUU | 8066 | AAAGAAGUCUUCGACCAAU |
| | | U | | |
| siRNA 1713 | 1713 | UUGGUCGAAGACUUCUUU | 8067 | CAAAGAAGUCUUCGACCAA |
| | | G | | |
| siRNA 1714 | 1714 | UGGUCGAAGACUUCUUUG | 8068 | ACAAAGAAGUCUUCGACCA |
| | | U | | |
| siRNA 1715 | 1715 | GGUCGAAGACUUCUUUGU | 8069 | CACAAAGAAGUCUUCGACC |
| | | G | | |
| siRNA 1716 | 1716 | GUCGAAGACUUCUUUGUG | 8070 | UCACAAAGAAGUCUUCGAC |
| | | A | | |
| siRNA 1717 | 1717 | UCGAAGACUUCUUUGUGA | 8071 | AUCACAAAGAAGUCUUCGA |
| | | U | | |
| siRNA 1718 | 1718 | CGAAGACUUCUUUGUGAU | 8072 | CAUCACAAAGAAGUCUUCG |
| | | G | | |
| siRNA 1719 | 1719 | GAAGACUUCUUUGUGAUG | 8073 | UCAUCACAAAGAAGUCUUC |
| | | A | | |
| siRNA 1720 | 1720 | AAGACUUCUUUGUGAUGA | 8074 | UUCAUCACAAAGAAGUCUU |
| | | A | | |
| siRNA 1721 | 1721 | AGACUUCUUUGUGAUGAA | 8075 | UUUCAUCACAAAGAAGUCU |
| | | A | | |
| siRNA 1722 | 1722 | GACUUCUUUGUGAUGAAA | 8076 | CUUUCAUCACAAAGAAGUC |
| | | G | | |
| siRNA 1723 | 1723 | ACUUCUUUGUGAUGAAAG | 8077 | UCUUUCAUCACAAAGAAGU |
| | | A | | |
| siRNA 1724 | 1724 | CUUCUUUGUGAUGAAAGA | 8078 | CUCUUUCAUCACAAAGAAG |
| | | G | | |
| siRNA 1725 | 1725 | UUCUUUGUGAUGAAAGAG | 8079 | CCUCUUUCAUCACAAAGAA |
| | | G | | |
| siRNA 1726 | 1726 | UCUUUGUGAUGAAAGAGG | 8080 | UCCUCUUUCAUCACAAAGA |
| | | A | | |
| siRNA 1727 | 1727 | CUUUGUGAUGAAAGAGGA | 8081 | UUCCUCUUUCAUCACAAAG |
| | | A | | |
| siRNA 1728 | 1728 | UUUGUGAUGAAAGAGGAA | 8082 | CUUCCUCUUUCAUCACAAA |
| | | G | | |
| siRNA 1729 | 1729 | UUGUGAUGAAAGAGGAAG | 8083 | ACUUCCUCUUUCAUCACAA |
| | | U | | |
| siRNA 1730 | 1730 | UGUGAUGAAAGAGGAAGU | 8084 | GACUUCCUCUUUCAUCACA |
| | | C | | |
| siRNA 1731 | 1731 | GUGAUGAAAGAGGAAGUC | 8085 | GGACUUCCUCUUUCAUCAC |
| | | C | | |
| siRNA 1732 | 1732 | UGAUGAAAGAGGAAGUCC | 8086 | AGGACUUCCUCUUUCAUCA |
| | | U | | |
| siRNA 1733 | 1733 | GAUGAAAGAGGAAGUCCU | 8087 | CAGGACUUCCUCUUUCAUC |
| | | G | | |
| siRNA 1734 | 1734 | AUGAAAGAGGAAGUCCUG | 8088 | CCAGGACUUCCUCUUUCAU |
| | | G | | |
| siRNA 1735 | 1735 | UGAAAGAGGAAGUCCUGG | 8089 | GCCAGGACUUCCUCUUUCA |
| | | C | | |
| siRNA 1736 | 1736 | GAAAGAGGAAGUCCUGGC | 8090 | AGCCAGGACUUCCUCUUUC |
| | | U | | |
| siRNA 1737 | 1737 | AAAGAGGAAGUCCUGGCUC | 8091 | GAGCCAGGACUUCCUCUUU |
| siRNA 1738 | 1738 | AAGAGGAAGUCCUGGCUCG | 8092 | CGAGCCAGGACUUCCUCUU |
| siRNA 1739 | 1739 | AGAGGAAGUCCUGGCUCGU | 8093 | ACGAGCCAGGACUUCCUCU |
| siRNA 1740 | 1740 | GAGGAAGUCCUGGCUCGUG | 8094 | CACGAGCCAGGACUUCCUC |
| siRNA 1741 | 1741 | AGGAAGUCCUGGCUCGUGA | 8095 | UCACGAGCCAGGACUUCCU |
| siRNA 1742 | 1742 | GGAAGUCCUGGCUCGUGAU | 8096 | AUCACGAGCCAGGACUUCC |
| siRNA 1743 | 1743 | GAAGUCCUGGCUCGUGAUU | 8097 | AAUCACGAGCCAGGACUUC |
| siRNA 1744 | 1744 | AAGUCCUGGCUCGUGAUUU | 8098 | AAAUCACGAGCCAGGACUU |
| siRNA 1745 | 1745 | AGUCCUGGCUCGUGAUUUU | 8099 | AAAAUCACGAGCCAGGACU |
| siRNA 1746 | 1746 | GUCCUGGCUCGUGAUUUUG | 8100 | CAAAAUCACGAGCCAGGAC |
| siRNA 1747 | 1747 | UCCUGGCUCGUGAUUUUGA | 8101 | UCAAAAUCACGAGCCAGGA |
| siRNA 1748 | 1748 | CCUGGCUCGUGAUUUUGAC | 8102 | GUCAAAAUCACGAGCCAGG |
| siRNA 1749 | 1749 | CUGGCUCGUGAUUUUGACC | 8103 | GGUCAAAAUCACGAGCCAG |
| siRNA 1750 | 1750 | UGGCUCGUGAUUUUGACCU | 8104 | AGGUCAAAAUCACGAGCCA |
| siRNA 1751 | 1751 | GGCUCGUGAUUUUGACCUG | 8105 | CAGGUCAAAAUCACGAGCC |
| siRNA 1752 | 1752 | GCUCGUGAUUUUGACCUGG | 8106 | CCAGGUCAAAAUCACGAGC |
| siRNA 1753 | 1753 | CUCGUGAUUUUGACCUGGG | 8107 | CCCAGGUCAAAAUCACGAG |
| siRNA 1754 | 1754 | UCGUGAUUUUGACCUGGG | 8108 | CCCCAGGUCAAAAUCACGA |
| | | G | | |
| siRNA 1755 | 1755 | CGUGAUUUUGACCUGGGG | 8109 | ACCCCAGGUCAAAAUCACG |
| | | U | | |
| siRNA 1756 | 1756 | GUGAUUUUGACCUGGGGU | 8110 | AACCCCAGGUCAAAAUCAC |
| | | U | | |
| siRNA 1757 | 1757 | UGAUUUUGACCUGGGGUU | 8111 | GAACCCCAGGUCAAAAUCA |
| | | C | | |
| siRNA 1758 | 1758 | GAUUUUGACCUGGGGUUC | 8112 | AGAACCCCAGGUCAAAAUC |
| | | U | | |
| siRNA 1759 | 1759 | AUUUUGACCUGGGGUUCUC | 8113 | GAGAACCCCAGGUCAAAAU |
| siRNA 1760 | 1760 | UUUUGACCUGGGGUUCUCA | 8114 | UGAGAACCCCAGGUCAAAA |
| siRNA 1761 | 1761 | UUUGACCUGGGGUUCUCAG | 8115 | CUGAGAACCCCAGGUCAAA |
| siRNA 1762 | 1762 | UUGACCUGGGGUUCUCAGG | 8116 | CCUGAGAACCCCAGGUCAA |
| siRNA 1763 | 1763 | UGACCUGGGGUUCUCAGGA | 8117 | UCCUGAGAACCCCAGGUCA |
| siRNA 1764 | 1764 | GACCUGGGGUUCUCAGGAA | 8118 | UUCCUGAGAACCCCAGGUC |
| siRNA 1765 | 1765 | ACCUGGGGUUCUCAGGAAA | 8119 | UUUCCUGAGAACCCCAGGU |
| siRNA 1766 | 1766 | CCUGGGGUUCUCAGGAAAU | 8120 | AUUUCCUGAGAACCCCAGG |
| siRNA 1767 | 1767 | CUGGGGUUCUCAGGAAAU | 8121 | AAUUUCCUGAGAACCCCAG |
| | | U | | |
| siRNA 1768 | 1768 | UGGGGUUCUCAGGAAAUU | 8122 | GAAUUUCCUGAGAACCCCA |
| | | C | | |
| siRNA 1769 | 1769 | GGGGUUCUCAGGAAAUUC | 8123 | UGAAUUUCCUGAGAACCCC |
| | | A | | |
| siRNA 1770 | 1770 | GGGUUCUCAGGAAAUUCA | 8124 | CUGAAUUUCCUGAGAACCC |
| | | G | | |
| siRNA 1771 | 1771 | GGUUCUCAGGAAAUUCAG | 8125 | UCUGAAUUUCCUGAGAACC |
| | | A | | |
| siRNA 1772 | 1772 | GUUCUCAGGAAAUUCAGA | 8126 | UUCUGAAUUUCCUGAGAAC |
| | | A | | |
| siRNA 1773 | 1773 | UUCUCAGGAAAUUCAGAA | 8127 | CUUCUGAAUUUCCUGAGAA |
| | | G | | |
| siRNA 1774 | 1774 | UCUCAGGAAAUUCAGAAG | 8128 | UCUUCUGAAUUUCCUGAGA |
| | | A | | |
| siRNA 1775 | 1775 | CUCAGGAAAUUCAGAAGA | 8129 | AUCUUCUGAAUUUCCUGAG |
| | | U | | |
| siRNA 1776 | 1776 | UCAGGAAAUUCAGAAGAU | 8130 | CAUCUUCUGAAUUUCCUGA |
| | | G | | |
| siRNA 1777 | 1777 | CAGGAAAUUCAGAAGAUG | 8131 | ACAUCUUCUGAAUUUCCUG |
| | | U | | |
| siRNA 1778 | 1778 | AGGAAAUUCAGAAGAUGU | 8132 | UACAUCUUCUGAAUUUCCU |
| | | A | | |
| siRNA 1779 | 1779 | GGAAAUUCAGAAGAUGUA | 8133 | CUACAUCUUCUGAAUUUCC |
| | | G | | |
| siRNA 1780 | 1780 | GAAAUUCAGAAGAUGUAG | 8134 | ACUACAUCUUCUGAAUUUC |
| | | U | | |
| siRNA 1781 | 1781 | AAAUUCAGAAGAUGUAGU | 8135 | UACUACAUCUUCUGAAUUU |
| | | A | | |
| siRNA 1782 | 1782 | AAUUCAGAAGAUGUAGUA | 8136 | UUACUACAUCUUCUGAAUU |
| | | A | | |
| siRNA 1783 | 1783 | AUUCAGAAGAUGUAGUAA | 8137 | AUUACUACAUCUUCUGAAU |
| | | U | | |
| siRNA 1784 | 1784 | UUCAGAAGAUGUAGUAAU | 8138 | CAUUACUACAUCUUCUGAA |
| | | G | | |
| siRNA 1785 | 1785 | UCAGAAGAUGUAGUAAUG | 8139 | GCAUUACUACAUCUUCUGA |
| | | C | | |
| siRNA 1786 | 1786 | CAGAAGAUGUAGUAAUGC | 8140 | UGCAUUACUACAUCUUCUG |
| | | A | | |
| siRNA 1787 | 1787 | AGAAGAUGUAGUAAUGCA | 8141 | AUGCAUUACUACAUCUUCU |
| | | U | | |
| siRNA 1788 | 1788 | GAAGAUGUAGUAAUGCAU | 8142 | CAUGCAUUACUACAUCUUC |
| | | G | | |
| siRNA 1789 | 1789 | AAGAUGUAGUAAUGCAUG | 8143 | GCAUGCAUUACUACAUCUU |
| | | C | | |
| siRNA 1790 | 1790 | AGAUGUAGUAAUGCAUGC | 8144 | GGCAUGCAUUACUACAUCU |
| | | C | | |
| siRNA 1791 | 1791 | GAUGUAGUAAUGCAUGCC | 8145 | UGGCAUGCAUUACUACAUC |
| | | A | | |
| siRNA 1792 | 1792 | AUGUAGUAAUGCAUGCCA | 8146 | AUGGCAUGCAUUACUACAU |
| | | U | | |
| siRNA 1793 | 1793 | UGUAGUAAUGCAUGCCAU | 8147 | UAUGGCAUGCAUUACUACA |
| | | A | | |
| siRNA 1794 | 1794 | GUAGUAAUGCAUGCCAUAC | 8148 | GUAUGGCAUGCAUUACUAC |
| siRNA 1795 | 1795 | UAGUAAUGCAUGCCAUACA | 8149 | UGUAUGGCAUGCAUUACU |
| | | | | A |
| siRNA 1796 | 1796 | AGUAAUGCAUGCCAUACAG | 8150 | CUGUAUGGCAUGCAUUACU |
| siRNA 1797 | 1797 | GUAAUGCAUGCCAUACAGC | 8151 | GCUGUAUGGCAUGCAUUAC |
| siRNA 1798 | 1798 | UAAUGCAUGCCAUACAGCU | 8152 | AGCUGUAUGGCAUGCAUU |
| | | | | A |
| siRNA 1799 | 1799 | AAUGCAUGCCAUACAGCUG | 8153 | CAGCUGUAUGGCAUGCAUU |
| siRNA 1800 | 1800 | AUGCAUGCCAUACAGCUGC | 8154 | GCAGCUGUAUGGCAUGCAU |
| siRNA 1801 | 1801 | UGCAUGCCAUACAGCUGCU | 8155 | AGCAGCUGUAUGGCAUGCA |
| siRNA 1802 | 1802 | GCAUGCCAUACAGCUGCUG | 8156 | CAGCAGCUGUAUGGCAUGC |
| siRNA 1803 | 1803 | CAUGCCAUACAGCUGCUGG | 8157 | CCAGCAGCUGUAUGGCAUG |
| siRNA 1804 | 1804 | AUGCCAUACAGCUGCUGGG | 8158 | CCCAGCAGCUGUAUGGCAU |
| siRNA 1805 | 1805 | UGCCAUACAGCUGCUGGGA | 8159 | UCCCAGCAGCUGUAUGGCA |
| siRNA 1806 | 1806 | GCCAUACAGCUGCUGGGAA | 8160 | UUCCCAGCAGCUGUAUGGC |
| siRNA 1807 | 1807 | CCAUACAGCUGCUGGGAAA | 8161 | UUUCCCAGCAGCUGUAUGG |
| siRNA 1808 | 1808 | CAUACAGCUGCUGGGAAAU | 8162 | AUUUCCCAGCAGCUGUAUG |
| siRNA 1809 | 1809 | AUACAGCUGCUGGGAAAU | 8163 | AAUUUCCCAGCAGCUGUAU |
| | | U | | |
| siRNA 1810 | 1810 | UACAGCUGCUGGGAAAUU | 8164 | CAAUUUCCCAGCAGCUGUA |
| | | G | | |
| siRNA 1811 | 1811 | ACAGCUGCUGGGAAAUUG | 8165 | ACAAUUUCCCAGCAGCUGU |
| | | U | | |
| siRNA 1812 | 1812 | CAGCUGCUGGGAAAUUGU | 8166 | CACAAUUUCCCAGCAGCUG |
| | | G | | |
| siRNA 1813 | 1813 | AGCUGCUGGGAAAUUGUG | 8167 | ACACAAUUUCCCAGCAGCU |
| | | U | | |
| siRNA 1814 | 1814 | GCUGCUGGGAAAUUGUGU | 8168 | GACACAAUUUCCCAGCAGC |
| | | C | | |
| siRNA 1815 | 1815 | CUGCUGGGAAAUUGUGUC | 8169 | UGACACAAUUUCCCAGCAG |
| | | A | | |
| siRNA 1816 | 1816 | UGCUGGGAAAUUGUGUCA | 8170 | GUGACACAAUUUCCCAGCA |
| | | C | | |
| siRNA 1817 | 1817 | GCUGGGAAAUUGUGUCAC | 8171 | UGUGACACAAUUUCCCAGC |
| | | A | | |
| siRNA 1818 | 1818 | CUGGGAAAUUGUGUCACA | 8172 | UUGUGACACAAUUUCCCAG |
| | | A | | |
| siRNA 1819 | 1819 | UGGGAAAUUGUGUCACAA | 8173 | AUUGUGACACAAUUUCCCA |
| | | U | | |
| siRNA 1820 | 1820 | GGGAAAUUGUGUCACAAU | 8174 | GAUUGUGACACAAUUUCCC |
| | | C | | |
| siRNA 1821 | 1821 | GGAAAUUGUGUCACAAUC | 8175 | UGAUUGUGACACAAUUUCC |
| | | A | | |
| siRNA 1822 | 1822 | GAAAUUGUGUCACAAUCAC | 8176 | GUGAUUGUGACACAAUUU |
| | | | | C |
| siRNA 1823 | 1823 | AAAUUGUGUCACAAUCACC | 8177 | GGUGAUUGUGACACAAUU |
| | | | | U |
| siRNA 1824 | 1824 | AAUUGUGUCACAAUCACCC | 8178 | GGGUGAUUGUGACACAAU |
| | | | | U |
| siRNA 1825 | 1825 | AUUGUGUCACAAUCACCCA | 8179 | UGGGUGAUUGUGACACAA |
| | | | | U |
| siRNA 1826 | 1826 | UUGUGUCACAAUCACCCAC | 8180 | GUGGGUGAUUGUGACACA |
| | | | | A |
| siRNA 1827 | 1827 | UGUGUCACAAUCACCCACA | 8181 | UGUGGGUGAUUGUGACAC |
| | | | | A |
| siRNA 1828 | 1828 | GUGUCACAAUCACCCACAC | 8182 | GUGUGGGUGAUUGUGACA |
| | | | | C |
| siRNA 1829 | 1829 | UGUCACAAUCACCCACACU | 8183 | AGUGUGGGUGAUUGUGAC |
| | | | | A |
| siRNA 1830 | 1830 | GUCACAAUCACCCACACUA | 8184 | UAGUGUGGGUGAUUGUGA |
| | | | | C |
| siRNA 1831 | 1831 | UCACAAUCACCCACACUAG | 8185 | CUAGUGUGGGUGAUUGUG |
| | | | | A |
| siRNA 1832 | 1832 | CACAAUCACCCACACUAGC | 8186 | GCUAGUGUGGGUGAUUGU |
| | | | | G |
| siRNA 1833 | 1833 | ACAAUCACCCACACUAGCA | 8187 | UGCUAGUGUGGGUGAUUG |
| | | | | U |
| siRNA 1834 | 1834 | CAAUCACCCACACUAGCAG | 8188 | CUGCUAGUGUGGGUGAUU |
| | | | | G |
| siRNA 1835 | 1835 | AAUCACCCACACUAGCAGG | 8189 | CCUGCUAGUGUGGGUGAU |
| | | | | U |
| siRNA 1836 | 1836 | AUCACCCACACUAGCAGGA | 8190 | UCCUGCUAGUGUGGGUGA |
| | | | | U |
| siRNA 1837 | 1837 | UCACCCACACUAGCAGGAA | 8191 | UUCCUGCUAGUGUGGGUG |
| | | | | A |
| siRNA 1838 | 1838 | CACCCACACUAGCAGGAAC | 8192 | GUUCCUGCUAGUGUGGGU |
| | | | | G |
| siRNA 1839 | 1839 | ACCCACACUAGCAGGAACG | 8193 | CGUUCCUGCUAGUGUGGGU |
| siRNA 1840 | 1840 | CCCACACUAGCAGGAACGA | 8194 | UCGUUCCUGCUAGUGUGGG |
| siRNA 1841 | 1841 | CCACACUAGCAGGAACGAU | 8195 | AUCGUUCCUGCUAGUGUGG |
| siRNA 1842 | 1842 | CACACUAGCAGGAACGAUG | 8196 | CAUCGUUCCUGCUAGUGUG |
| siRNA 1843 | 1843 | ACACUAGCAGGAACGAUGA | 8197 | UCAUCGUUCCUGCUAGUGU |
| siRNA 1844 | 1844 | CACUAGCAGGAACGAUGAG | 8198 | CUCAUCGUUCCUGCUAGUG |
| siRNA 1845 | 1845 | ACUAGCAGGAACGAUGAG | 8199 | ACUCAUCGUUCCUGCUAGU |
| | | U | | |
| siRNA 1846 | 1846 | CUAGCAGGAACGAUGAGU | 8200 | AACUCAUCGUUCCUGCUAG |
| | | U | | |
| siRNA 1847 | 1847 | UAGCAGGAACGAUGAGUU | 8201 | AAACUCAUCGUUCCUGCUA |
| | | U | | |
| siRNA 1848 | 1848 | AGCAGGAACGAUGAGUUU | 8202 | AAAACUCAUCGUUCCUGCU |
| | | U | | |
| siRNA 1849 | 1849 | GCAGGAACGAUGAGUUUU | 8203 | AAAAACUCAUCGUUCCUGC |
| | | U | | |
| siRNA 1850 | 1850 | CAGGAACGAUGAGUUUUU | 8204 | AAAAAACUCAUCGUUCCUG |
| | | U | | |
| siRNA 1851 | 1851 | AGGAACGAUGAGUUUUUU | 8205 | UAAAAAACUCAUCGUUCCU |
| | | A | | |
| siRNA 1852 | 1852 | GGAACGAUGAGUUUUUUA | 8206 | AUAAAAAACUCAUCGUUCC |
| | | U | | |
| siRNA 1853 | 1853 | GAACGAUGAGUUUUUUAU | 8207 | GAUAAAAAACUCAUCGUUC |
| | | C | | |
| siRNA 1854 | 1854 | AACGAUGAGUUUUUUAUC | 8208 | UGAUAAAAAACUCAUCGU |
| | | A | | U |
| siRNA 1855 | 1855 | ACGAUGAGUUUUUUAUCA | 8209 | GUGAUAAAAAACUCAUCG |
| | | C | | U |
| siRNA 1856 | 1856 | CGAUGAGUUUUUUAUCACC | 8210 | GGUGAUAAAAAACUCAUC |
| | | | | G |
| siRNA 1857 | 1857 | GAUGAGUUUUUUAUCACCC | 8211 | GGGUGAUAAAAAACUCAU |
| | | | | C |
| siRNA 1858 | 1858 | AUGAGUUUUUUAUCACCCC | 8212 | GGGGUGAUAAAAAACUCA |
| | | | | U |
| siRNA 1859 | 1859 | UGAGUUUUUUAUCACCCCC | 8213 | GGGGGUGAUAAAAAACUC |
| | | | | A |
| siRNA 1860 | 1860 | GAGUUUUUUAUCACCCCCA | 8214 | UGGGGGUGAUAAAAAACU |
| | | | | C |
| siRNA 1861 | 1861 | AGUUUUUUAUCACCCCCAG | 8215 | CUGGGGGUGAUAAAAAAC |
| | | | | U |
| siRNA 1862 | 1862 | GUUUUUUAUCACCCCCAGC | 8216 | GCUGGGGGUGAUAAAAAA |
| | | | | C |
| siRNA 1863 | 1863 | UUUUUUAUCACCCCCAGCA | 8217 | UGCUGGGGGUGAUAAAAA |
| | | | | A |
| siRNA 1864 | 1864 | UUUUUAUCACCCCCAGCAC | 8218 | GUGCUGGGGGUGAUAAAA |
| | | | | A |
| siRNA 1865 | 1865 | UUUUAUCACCCCCAGCACA | 8219 | UGUGCUGGGGGUGAUAAA |
| | | | | A |
| siRNA 1866 | 1866 | UUUAUCACCCCCAGCACAA | 8220 | UUGUGCUGGGGGUGAUAA |
| | | | | A |
| siRNA 1867 | 1867 | UUAUCACCCCCAGCACAAC | 8221 | GUUGUGCUGGGGGUGAUA |
| | | | | A |
| siRNA 1868 | 1868 | UAUCACCCCCAGCACAACU | 8222 | AGUUGUGCUGGGGGUGAU |
| | | | | A |
| siRNA 1869 | 1869 | AUCACCCCCAGCACAACUG | 8223 | CAGUUGUGCUGGGGGUGA |
| | | | | U |
| siRNA 1870 | 1870 | UCACCCCCAGCACAACUGU | 8224 | ACAGUUGUGCUGGGGGUG |
| | | | | A |
| siRNA 1871 | 1871 | CACCCCCAGCACAACUGUC | 8225 | GACAGUUGUGCUGGGGGU |
| | | | | G |
| siRNA 1872 | 1872 | ACCCCCAGCACAACUGUCC | 8226 | GGACAGUUGUGCUGGGGG |
| | | | | U |
| siRNA 1873 | 1873 | CCCCCAGCACAACUGUCCC | 8227 | GGGACAGUUGUGCUGGGG |
| | | | | G |
| siRNA 1874 | 1874 | CCCCAGCACAACUGUCCCA | 8228 | UGGGACAGUUGUGCUGGG |
| | | | | G |
| siRNA 1875 | 1875 | CCCAGCACAACUGUCCCAU | 8229 | AUGGGACAGUUGUGCUGG |
| | | | | G |
| siRNA 1876 | 1876 | CCAGCACAACUGUCCCAUC | 8230 | GAUGGGACAGUUGUGCUG |
| | | | | G |
| siRNA 1877 | 1877 | CAGCACAACUGUCCCAUCA | 8231 | UGAUGGGACAGUUGUGCU |
| | | | | G |
| siRNA 1878 | 1878 | AGCACAACUGUCCCAUCAG | 8232 | CUGAUGGGACAGUUGUGC |
| | | | | U |
| siRNA 1879 | 1879 | GCACAACUGUCCCAUCAGU | 8233 | ACUGAUGGGACAGUUGUG |
| | | | | C |
| siRNA 1880 | 1880 | CACAACUGUCCCAUCAGUC | 8234 | GACUGAUGGGACAGUUGU |
| | | | | G |
| siRNA 1881 | 1881 | ACAACUGUCCCAUCAGUCU | 8235 | AGACUGAUGGGACAGUUG |
| | | | | U |
| siRNA 1882 | 1882 | CAACUGUCCCAUCAGUCUU | 8236 | AAGACUGAUGGGACAGUU |
| | | | | G |
| siRNA 1883 | 1883 | AACUGUCCCAUCAGUCUUC | 8237 | GAAGACUGAUGGGACAGU |
| | | | | U |
| siRNA 1884 | 1884 | ACUGUCCCAUCAGUCUUCG | 8238 | CGAAGACUGAUGGGACAG |
| | | | | U |
| siRNA 1885 | 1885 | CUGUCCCAUCAGUCUUCGA | 8239 | UCGAAGACUGAUGGGACA |
| | | | | G |
| siRNA 1886 | 1886 | UGUCCCAUCAGUCUUCGAA | 8240 | UUCGAAGACUGAUGGGAC |
| | | | | A |
| siRNA 1887 | 1887 | GUCCCAUCAGUCUUCGAAC | 8241 | GUUCGAAGACUGAUGGGA |
| | | | | C |
| siRNA 1888 | 1888 | UCCCAUCAGUCUUCGAACU | 8242 | AGUUCGAAGACUGAUGGG |
| | | | | A |
| siRNA 1889 | 1889 | CCCAUCAGUCUUCGAACUC | 8243 | GAGUUCGAAGACUGAUGG |
| | | | | G |
| siRNA 1890 | 1890 | CCAUCAGUCUUCGAACUCA | 8244 | UGAGUUCGAAGACUGAUG |
| | | | | G |
| siRNA 1891 | 1891 | CAUCAGUCUUCGAACUCAA | 8245 | UUGAGUUCGAAGACUGAU |
| | | | | G |
| siRNA 1892 | 1892 | AUCAGUCUUCGAACUCAAC | 8246 | GUUGAGUUCGAAGACUGA |
| | | | | U |
| siRNA 1893 | 1893 | UCAGUCUUCGAACUCAACU | 8247 | AGUUGAGUUCGAAGACUG |
| | | | | A |
| siRNA 1894 | 1894 | CAGUCUUCGAACUCAACUU | 8248 | AAGUUGAGUUCGAAGACU |
| | | | | G |
| siRNA 1895 | 1895 | AGUCUUCGAACUCAACUUC | 8249 | GAAGUUGAGUUCGAAGAC |
| | | | | U |
| siRNA 1896 | 1896 | GUCUUCGAACUCAACUUCU | 8250 | AGAAGUUGAGUUCGAAGA |
| | | | | C |
| siRNA 1897 | 1897 | UCUUCGAACUCAACUUCUA | 8251 | UAGAAGUUGAGUUCGAAG |
| | | | | A |
| siRNA 1898 | 1898 | CUUCGAACUCAACUUCUAC | 8252 | GUAGAAGUUGAGUUCGAA |
| | | | | G |
| siRNA 1899 | 1899 | UUCGAACUCAACUUCUACA | 8253 | UGUAGAAGUUGAGUUCGA |
| | | | | A |
| siRNA 1900 | 1900 | UCGAACUCAACUUCUACAG | 8254 | CUGUAGAAGUUGAGUUCG |
| | | | | A |
| siRNA 1901 | 1901 | CGAACUCAACUUCUACAGC | 8255 | GCUGUAGAAGUUGAGUUC |
| | | | | G |
| siRNA 1902 | 1902 | GAACUCAACUUCUACAGCA | 8256 | UGCUGUAGAAGUUGAGUU |
| | | | | C |
| siRNA 1903 | 1903 | AACUCAACUUCUACAGCAA | 8257 | UUGCUGUAGAAGUUGAGU |
| | | | | U |
| siRNA 1904 | 1904 | ACUCAACUUCUACAGCAAU | 8258 | AUUGCUGUAGAAGUUGAG |
| | | | | U |
| siRNA 1905 | 1905 | CUCAACUUCUACAGCAAUG | 8259 | CAUUGCUGUAGAAGUUGA |
| | | | | G |
| siRNA 1906 | 1906 | UCAACUUCUACAGCAAUGG | 8260 | CCAUUGCUGUAGAAGUUG |
| | | | | A |
| siRNA 1907 | 1907 | CAACUUCUACAGCAAUGGG | 8261 | CCCAUUGCUGUAGAAGUUG |
| siRNA 1908 | 1908 | AACUUCUACAGCAAUGGGG | 8262 | CCCCAUUGCUGUAGAAGUU |
| siRNA 1909 | 1909 | ACUUCUACAGCAAUGGGGU | 8263 | ACCCCAUUGCUGUAGAAGU |
| siRNA 1910 | 1910 | CUUCUACAGCAAUGGGGUA | 8264 | UACCCCAUUGCUGUAGAAG |
| siRNA 1911 | 1911 | UUCUACAGCAAUGGGGUAC | 8265 | GUACCCCAUUGCUGUAGAA |
| siRNA 1912 | 1912 | UCUACAGCAAUGGGGUACU | 8266 | AGUACCCCAUUGCUGUAGA |
| siRNA 1913 | 1913 | CUACAGCAAUGGGGUACUU | 8267 | AAGUACCCCAUUGCUGUAG |
| siRNA 1914 | 1914 | UACAGCAAUGGGGUACUUC | 8268 | GAAGUACCCCAUUGCUGUA |
| siRNA 1915 | 1915 | ACAGCAAUGGGGUACUUCA | 8269 | UGAAGUACCCCAUUGCUGU |
| siRNA 1916 | 1916 | CAGCAAUGGGGUACUUCAU | 8270 | AUGAAGUACCCCAUUGCUG |
| siRNA 1917 | 1917 | AGCAAUGGGGUACUUCAU | 8271 | CAUGAAGUACCCCAUUGCU |
| | | G | | |
| siRNA 1918 | 1918 | GCAAUGGGGUACUUCAUG | 8272 | ACAUGAAGUACCCCAUUGC |
| | | U | | |
| siRNA 1919 | 1919 | CAAUGGGGUACUUCAUGUC | 8273 | GACAUGAAGUACCCCAUUG |
| siRNA 1920 | 1920 | AAUGGGGUACUUCAUGUC | 8274 | AGACAUGAAGUACCCCAUU |
| | | U | | |
| siRNA 1921 | 1921 | AUGGGGUACUUCAUGUCU | 8275 | AAGACAUGAAGUACCCCAU |
| | | U | | |
| siRNA 1922 | 1922 | UGGGGUACUUCAUGUCUU | 8276 | AAAGACAUGAAGUACCCCA |
| | | U | | |
| siRNA 1923 | 1923 | GGGGUACUUCAUGUCUUU | 8277 | UAAAGACAUGAAGUACCCC |
| | | A | | |
| siRNA 1924 | 1924 | GGGUACUUCAUGUCUUUA | 8278 | AUAAAGACAUGAAGUACCC |
| | | U | | |
| siRNA 1925 | 1925 | GGUACUUCAUGUCUUUAUC | 8279 | GAUAAAGACAUGAAGUAC |
| | | | | C |
| siRNA 1926 | 1926 | GUACUUCAUGUCUUUAUCA | 8280 | UGAUAAAGACAUGAAGUA |
| | | | | C |
| siRNA 1927 | 1927 | UACUUCAUGUCUUUAUCAU | 8281 | AUGAUAAAGACAUGAAGU |
| | | | | A |
| siRNA 1928 | 1928 | ACUUCAUGUCUUUAUCAUG | 8282 | CAUGAUAAAGACAUGAAG |
| | | | | U |
| siRNA 1929 | 1929 | CUUCAUGUCUUUAUCAUGG | 8283 | CCAUGAUAAAGACAUGAA |
| | | | | G |
| siRNA 1930 | 1930 | UUCAUGUCUUUAUCAUGG | 8284 | UCCAUGAUAAAGACAUGA |
| | | A | | A |
| siRNA 1931 | 1931 | UCAUGUCUUUAUCAUGGA | 8285 | CUCCAUGAUAAAGACAUGA |
| | | G | | |
| siRNA 1932 | 1932 | CAUGUCUUUAUCAUGGAG | 8286 | CCUCCAUGAUAAAGACAUG |
| | | G | | |
| siRNA 1933 | 1933 | AUGUCUUUAUCAUGGAGG | 8287 | GCCUCCAUGAUAAAGACAU |
| | | C | | |
| siRNA 1934 | 1934 | UGUCUUUAUCAUGGAGGCC | 8288 | GGCCUCCAUGAUAAAGACA |
| siRNA 1935 | 1935 | GUCUUUAUCAUGGAGGCCA | 8289 | UGGCCUCCAUGAUAAAGAC |
| siRNA 1936 | 1936 | UCUUUAUCAUGGAGGCCAU | 8290 | AUGGCCUCCAUGAUAAAGA |
| siRNA 1937 | 1937 | CUUUAUCAUGGAGGCCAUC | 8291 | GAUGGCCUCCAUGAUAAAG |
| siRNA 1938 | 1938 | UUUAUCAUGGAGGCCAUCA | 8292 | UGAUGGCCUCCAUGAUAAA |
| siRNA 1939 | 1939 | UUAUCAUGGAGGCCAUCAU | 8293 | AUGAUGGCCUCCAUGAUAA |
| siRNA 1940 | 1940 | UAUCAUGGAGGCCAUCAUA | 8294 | UAUGAUGGCCUCCAUGAUA |
| siRNA 1941 | 1941 | AUCAUGGAGGCCAUCAUAG | 8295 | CUAUGAUGGCCUCCAUGAU |
| siRNA 1942 | 1942 | UCAUGGAGGCCAUCAUAGC | 8296 | GCUAUGAUGGCCUCCAUGA |
| siRNA 1943 | 1943 | CAUGGAGGCCAUCAUAGCU | 8297 | AGCUAUGAUGGCCUCCAUG |
| siRNA 1944 | 1944 | AUGGAGGCCAUCAUAGCUU | 8298 | AAGCUAUGAUGGCCUCCAU |
| siRNA 1945 | 1945 | UGGAGGCCAUCAUAGCUUG | 8299 | CAAGCUAUGAUGGCCUCCA |
| siRNA 1946 | 1946 | GGAGGCCAUCAUAGCUUGC | 8300 | GCAAGCUAUGAUGGCCUCC |
| siRNA 1947 | 1947 | GAGGCCAUCAUAGCUUGCA | 8301 | UGCAAGCUAUGAUGGCCUC |
| siRNA 1948 | 1948 | AGGCCAUCAUAGCUUGCAG | 8302 | CUGCAAGCUAUGAUGGCCU |
| siRNA 1949 | 1949 | GGCCAUCAUAGCUUGCAGC | 8303 | GCUGCAAGCUAUGAUGGCC |
| siRNA 1950 | 1950 | GCCAUCAUAGCUUGCAGCC | 8304 | GGCUGCAAGCUAUGAUGGC |
| siRNA 1951 | 1951 | CCAUCAUAGCUUGCAGCCU | 8305 | AGGCUGCAAGCUAUGAUG |
| | | | | G |
| siRNA 1952 | 1952 | CAUCAUAGCUUGCAGCCUU | 8306 | AAGGCUGCAAGCUAUGAU |
| | | | | G |
| siRNA 1953 | 1953 | AUCAUAGCUUGCAGCCUUU | 8307 | AAAGGCUGCAAGCUAUGA |
| | | | | U |
| siRNA 1954 | 1954 | UCAUAGCUUGCAGCCUUUA | 8308 | UAAAGGCUGCAAGCUAUG |
| | | | | A |
| siRNA 1955 | 1955 | CAUAGCUUGCAGCCUUUAU | 8309 | AUAAAGGCUGCAAGCUAU |
| | | | | G |
| siRNA 1956 | 1956 | AUAGCUUGCAGCCUUUAUG | 8310 | CAUAAAGGCUGCAAGCUAU |
| siRNA 1957 | 1957 | UAGCUUGCAGCCUUUAUGC | 8311 | GCAUAAAGGCUGCAAGCUA |
| siRNA 1958 | 1958 | AGCUUGCAGCCUUUAUGCA | 8312 | UGCAUAAAGGCUGCAAGCU |
| siRNA 1959 | 1959 | GCUUGCAGCCUUUAUGCAG | 8313 | CUGCAUAAAGGCUGCAAGC |
| siRNA 1960 | 1960 | CUUGCAGCCUUUAUGCAGU | 8314 | ACUGCAUAAAGGCUGCAAG |
| siRNA 1961 | 1961 | UUGCAGCCUUUAUGCAGUU | 8315 | AACUGCAUAAAGGCUGCAA |
| siRNA 1962 | 1962 | UGCAGCCUUUAUGCAGUUC | 8316 | GAACUGCAUAAAGGCUGCA |
| siRNA 1963 | 1963 | GCAGCCUUUAUGCAGUUCU | 8317 | AGAACUGCAUAAAGGCUGC |
| siRNA 1964 | 1964 | CAGCCUUUAUGCAGUUCUG | 8318 | CAGAACUGCAUAAAGGCUG |
| siRNA 1965 | 1965 | AGCCUUUAUGCAGUUCUGA | 8319 | UCAGAACUGCAUAAAGGCU |
| siRNA 1966 | 1966 | GCCUUUAUGCAGUUCUGAA | 8320 | UUCAGAACUGCAUAAAGGC |
| siRNA 1967 | 1967 | CCUUUAUGCAGUUCUGAAC | 8321 | GUUCAGAACUGCAUAAAG |
| | | | | G |
| siRNA 1968 | 1968 | CUUUAUGCAGUUCUGAACA | 8322 | UGUUCAGAACUGCAUAAA |
| | | | | G |
| siRNA 1969 | 1969 | UUUAUGCAGUUCUGAACA | 8323 | UUGUUCAGAACUGCAUAA |
| | | A | | A |
| siRNA 1970 | 1970 | UUAUGCAGUUCUGAACAA | 8324 | CUUGUUCAGAACUGCAUAA |
| | | G | | |
| siRNA 1971 | 1971 | UAUGCAGUUCUGAACAAG | 8325 | UCUUGUUCAGAACUGCAUA |
| | | A | | |
| siRNA 1972 | 1972 | AUGCAGUUCUGAACAAGA | 8326 | CUCUUGUUCAGAACUGCAU |
| | | G | | |
| siRNA 1973 | 1973 | UGCAGUUCUGAACAAGAG | 8327 | CCUCUUGUUCAGAACUGCA |
| | | G | | |
| siRNA 1974 | 1974 | GCAGUUCUGAACAAGAGG | 8328 | CCCUCUUGUUCAGAACUGC |
| | | G | | |
| siRNA 1975 | 1975 | CAGUUCUGAACAAGAGGG | 8329 | CCCCUCUUGUUCAGAACUG |
| | | G | | |
| siRNA 1976 | 1976 | AGUUCUGAACAAGAGGGG | 8330 | UCCCCUCUUGUUCAGAACU |
| | | A | | |
| siRNA 1977 | 1977 | GUUCUGAACAAGAGGGGA | 8331 | GUCCCCUCUUGUUCAGAAC |
| | | C | | |
| siRNA 1978 | 1978 | UUCUGAACAAGAGGGGAC | 8332 | AGUCCCCUCUUGUUCAGAA |
| | | U | | |
| siRNA 1979 | 1979 | UCUGAACAAGAGGGGACU | 8333 | CAGUCCCCUCUUGUUCAGA |
| | | G | | |
| siRNA 1980 | 1980 | CUGAACAAGAGGGGACUG | 8334 | CCAGUCCCCUCUUGUUCAG |
| | | G | | |
| siRNA 1981 | 1981 | UGAACAAGAGGGGACUGG | 8335 | CCCAGUCCCCUCUUGUUCA |
| | | G | | |
| siRNA 1982 | 1982 | GAACAAGAGGGGACUGGG | 8336 | CCCCAGUCCCCUCUUGUUC |
| | | G | | |
| siRNA 1983 | 1983 | AACAAGAGGGGACUGGGG | 8337 | CCCCCAGUCCCCUCUUGUU |
| | | G | | |
| siRNA 1984 | 1984 | ACAAGAGGGGACUGGGGG | 8338 | CCCCCCAGUCCCCUCUUGU |
| | | G | | |
| siRNA 1985 | 1985 | CAAGAGGGGACUGGGGGG | 8339 | ACCCCCCAGUCCCCUCUUG |
| | | U | | |
| siRNA 1986 | 1986 | AAGAGGGGACUGGGGGGU | 8340 | GACCCCCCAGUCCCCUCUU |
| | | C | | |
| siRNA 1987 | 1987 | AGAGGGGACUGGGGGGUC | 8341 | GGACCCCCCAGUCCCCUCU |
| | | C | | |
| siRNA 1988 | 1988 | GAGGGGACUGGGGGGUCCC | 8342 | GGGACCCCCCAGUCCCCUC |
| siRNA 1989 | 1989 | AGGGGACUGGGGGGUCCCA | 8343 | UGGGACCCCCCAGUCCCCU |
| siRNA 1990 | 1990 | GGGGACUGGGGGGUCCCAC | 8344 | GUGGGACCCCCCAGUCCCC |
| siRNA 1991 | 1991 | GGGACUGGGGGGUCCCACU | 8345 | AGUGGGACCCCCCAGUCCC |
| siRNA 1992 | 1992 | GGACUGGGGGGUCCCACUA | 8346 | UAGUGGGACCCCCCAGUCC |
| siRNA 1993 | 1993 | GACUGGGGGGUCCCACUAG | 8347 | CUAGUGGGACCCCCCAGUC |
| siRNA 1994 | 1994 | ACUGGGGGGUCCCACUAGC | 8348 | GCUAGUGGGACCCCCCAGU |
| siRNA 1995 | 1995 | CUGGGGGGUCCCACUAGCA | 8349 | UGCUAGUGGGACCCCCCAG |
| siRNA 1996 | 1996 | UGGGGGGUCCCACUAGCAC | 8350 | GUGCUAGUGGGACCCCCCA |
| siRNA 1997 | 1997 | GGGGGGUCCCACUAGCACC | 8351 | GGUGCUAGUGGGACCCCCC |
| siRNA 1998 | 1998 | GGGGGUCCCACUAGCACCC | 8352 | GGGUGCUAGUGGGACCCCC |
| siRNA 1999 | 1999 | GGGGUCCCACUAGCACCCC | 8353 | GGGGUGCUAGUGGGACCCC |
| siRNA 2000 | 2000 | GGGUCCCACUAGCACCCCA | 8354 | UGGGGUGCUAGUGGGACCC |
| siRNA 2001 | 2001 | GGUCCCACUAGCACCCCAC | 8355 | GUGGGGUGCUAGUGGGAC |
| | | | | C |
| siRNA 2002 | 2002 | GUCCCACUAGCACCCCACC | 8356 | GGUGGGGUGCUAGUGGGA |
| | | | | C |
| siRNA 2003 | 2003 | UCCCACUAGCACCCCACCU | 8357 | AGGUGGGGUGCUAGUGGG |
| | | | | A |
| siRNA 2004 | 2004 | CCCACUAGCACCCCACCUA | 8358 | UAGGUGGGGUGCUAGUGG |
| | | | | G |
| siRNA 2005 | 2005 | CCACUAGCACCCCACCUAA | 8359 | UUAGGUGGGGUGCUAGUG |
| | | | | G |
| siRNA 2006 | 2006 | CACUAGCACCCCACCUAAC | 8360 | GUUAGGUGGGGUGCUAGU |
| | | | | G |
| siRNA 2007 | 2007 | ACUAGCACCCCACCUAACC | 8361 | GGUUAGGUGGGGUGCUAG |
| | | | | U |
| siRNA 2008 | 2008 | CUAGCACCCCACCUAACCU | 8362 | AGGUUAGGUGGGGUGCUA |
| | | | | G |
| siRNA 2009 | 2009 | UAGCACCCCACCUAACCUG | 8363 | CAGGUUAGGUGGGGUGCU |
| | | | | A |
| siRNA 2010 | 2010 | AGCACCCCACCUAACCUGA | 8364 | UCAGGUUAGGUGGGGUGC |
| | | | | U |
| siRNA 2011 | 2011 | GCACCCCACCUAACCUGAU | 8365 | AUCAGGUUAGGUGGGGUG |
| | | | | C |
| siRNA 2012 | 2012 | CACCCCACCUAACCUGAUC | 8366 | GAUCAGGUUAGGUGGGGU |
| | | | | G |
| siRNA 2013 | 2013 | ACCCCACCUAACCUGAUCA | 8367 | UGAUCAGGUUAGGUGGGG |
| | | | | U |
| siRNA 2014 | 2014 | CCCCACCUAACCUGAUCAG | 8368 | CUGAUCAGGUUAGGUGGG |
| | | | | G |
| siRNA 2015 | 2015 | CCCACCUAACCUGAUCAGC | 8369 | GCUGAUCAGGUUAGGUGG |
| | | | | G |
| siRNA 2016 | 2016 | CCACCUAACCUGAUCAGCC | 8370 | GGCUGAUCAGGUUAGGUG |
| | | | | G |
| siRNA 2017 | 2017 | CACCUAACCUGAUCAGCCA | 8371 | UGGCUGAUCAGGUUAGGU |
| | | | | G |
| siRNA 2018 | 2018 | ACCUAACCUGAUCAGCCAG | 8372 | CUGGCUGAUCAGGUUAGG |
| | | | | U |
| siRNA 2019 | 2019 | CCUAACCUGAUCAGCCAGG | 8373 | CCUGGCUGAUCAGGUUAGG |
| siRNA 2020 | 2020 | CUAACCUGAUCAGCCAGGA | 8374 | UCCUGGCUGAUCAGGUUAG |
| siRNA 2021 | 2021 | UAACCUGAUCAGCCAGGAG | 8375 | CUCCUGGCUGAUCAGGUUA |
| siRNA 2022 | 2022 | AACCUGAUCAGCCAGGAGC | 8376 | GCUCCUGGCUGAUCAGGUU |
| siRNA 2023 | 2023 | ACCUGAUCAGCCAGGAGCA | 8377 | UGCUCCUGGCUGAUCAGGU |
| siRNA 2024 | 2024 | CCUGAUCAGCCAGGAGCAG | 8378 | CUGCUCCUGGCUGAUCAGG |
| siRNA 2025 | 2025 | CUGAUCAGCCAGGAGCAGC | 8379 | GCUGCUCCUGGCUGAUCAG |
| siRNA 2026 | 2026 | UGAUCAGCCAGGAGCAGCU | 8380 | AGCUGCUCCUGGCUGAUCA |
| siRNA 2027 | 2027 | GAUCAGCCAGGAGCAGCUG | 8381 | CAGCUGCUCCUGGCUGAUC |
| siRNA 2028 | 2028 | AUCAGCCAGGAGCAGCUGG | 8382 | CCAGCUGCUCCUGGCUGAU |
| siRNA 2029 | 2029 | UCAGCCAGGAGCAGCUGGU | 8383 | ACCAGCUGCUCCUGGCUGA |
| siRNA 2030 | 2030 | CAGCCAGGAGCAGCUGGUG | 8384 | CACCAGCUGCUCCUGGCUG |
| siRNA 2031 | 2031 | AGCCAGGAGCAGCUGGUGC | 8385 | GCACCAGCUGCUCCUGGCU |
| siRNA 2032 | 2032 | GCCAGGAGCAGCUGGUGCG | 8386 | CGCACCAGCUGCUCCUGGC |
| siRNA 2033 | 2033 | CCAGGAGCAGCUGGUGCGG | 8387 | CCGCACCAGCUGCUCCUGG |
| siRNA 2034 | 2034 | CAGGAGCAGCUGGUGCGGA | 8388 | UCCGCACCAGCUGCUCCUG |
| siRNA 2035 | 2035 | AGGAGCAGCUGGUGCGGA | 8389 | UUCCGCACCAGCUGCUCCU |
| | | A | | |
| siRNA 2036 | 2036 | GGAGCAGCUGGUGCGGAA | 8390 | CUUCCGCACCAGCUGCUCC |
| | | G | | |
| siRNA 2037 | 2037 | GAGCAGCUGGUGCGGAAG | 8391 | CCUUCCGCACCAGCUGCUC |
| | | G | | |
| siRNA 2038 | 2038 | AGCAGCUGGUGCGGAAGGC | 8392 | GCCUUCCGCACCAGCUGCU |
| siRNA 2039 | 2039 | GCAGCUGGUGCGGAAGGCG | 8393 | CGCCUUCCGCACCAGCUGC |
| siRNA 2040 | 2040 | CAGCUGGUGCGGAAGGCGG | 8394 | CCGCCUUCCGCACCAGCUG |
| siRNA 2041 | 2041 | AGCUGGUGCGGAAGGCGGC | 8395 | GCCGCCUUCCGCACCAGCU |
| siRNA 2042 | 2042 | GCUGGUGCGGAAGGCGGCC | 8396 | GGCCGCCUUCCGCACCAGC |
| siRNA 2043 | 2043 | CUGGUGCGGAAGGCGGCCA | 8397 | UGGCCGCCUUCCGCACCAG |
| siRNA 2044 | 2044 | UGGUGCGGAAGGCGGCCAG | 8398 | CUGGCCGCCUUCCGCACCA |
| siRNA 2045 | 2045 | GGUGCGGAAGGCGGCCAGC | 8399 | GCUGGCCGCCUUCCGCACC |
| siRNA 2046 | 2046 | GUGCGGAAGGCGGCCAGCC | 8400 | GGCUGGCCGCCUUCCGCAC |
| siRNA 2047 | 2047 | UGCGGAAGGCGGCCAGCCU | 8401 | AGGCUGGCCGCCUUCCGCA |
| siRNA 2048 | 2048 | GCGGAAGGCGGCCAGCCUG | 8402 | CAGGCUGGCCGCCUUCCGC |
| siRNA 2049 | 2049 | CGGAAGGCGGCCAGCCUGU | 8403 | ACAGGCUGGCCGCCUUCCG |
| siRNA 2050 | 2050 | GGAAGGCGGCCAGCCUGUG | 8404 | CACAGGCUGGCCGCCUUCC |
| siRNA 2051 | 2051 | GAAGGCGGCCAGCCUGUGC | 8405 | GCACAGGCUGGCCGCCUUC |
| siRNA 2052 | 2052 | AAGGCGGCCAGCCUGUGCU | 8406 | AGCACAGGCUGGCCGCCUU |
| siRNA 2053 | 2053 | AGGCGGCCAGCCUGUGCUA | 8407 | UAGCACAGGCUGGCCGCCU |
| siRNA 2054 | 2054 | GGCGGCCAGCCUGUGCUAC | 8408 | GUAGCACAGGCUGGCCGCC |
| siRNA 2055 | 2055 | GCGGCCAGCCUGUGCUACC | 8409 | GGUAGCACAGGCUGGCCGC |
| siRNA 2056 | 2056 | CGGCCAGCCUGUGCUACCU | 8410 | AGGUAGCACAGGCUGGCCG |
| siRNA 2057 | 2057 | GGCCAGCCUGUGCUACCUU | 8411 | AAGGUAGCACAGGCUGGCC |
| siRNA 2058 | 2058 | GCCAGCCUGUGCUACCUUC | 8412 | GAAGGUAGCACAGGCUGGC |
| siRNA 2059 | 2059 | CCAGCCUGUGCUACCUUCU | 8413 | AGAAGGUAGCACAGGCUG |
| | | | | G |
| siRNA 2060 | 2060 | CAGCCUGUGCUACCUUCUC | 8414 | GAGAAGGUAGCACAGGCU |
| | | | | G |
| siRNA 2061 | 2061 | AGCCUGUGCUACCUUCUCU | 8415 | AGAGAAGGUAGCACAGGC |
| | | | | U |
| siRNA 2062 | 2062 | GCCUGUGCUACCUUCUCUC | 8416 | GAGAGAAGGUAGCACAGG |
| | | | | C |
| siRNA 2063 | 2063 | CCUGUGCUACCUUCUCUCC | 8417 | GGAGAGAAGGUAGCACAG |
| | | | | G |
| siRNA 2064 | 2064 | CUGUGCUACCUUCUCUCCA | 8418 | UGGAGAGAAGGUAGCACA |
| | | | | G |
| siRNA 2065 | 2065 | UGUGCUACCUUCUCUCCAA | 8419 | UUGGAGAGAAGGUAGCAC |
| | | | | A |
| siRNA 2066 | 2066 | GUGCUACCUUCUCUCCAAU | 8420 | AUUGGAGAGAAGGUAGCA |
| | | | | C |
| siRNA 2067 | 2067 | UGCUACCUUCUCUCCAAUG | 8421 | CAUUGGAGAGAAGGUAGC |
| | | | | A |
| siRNA 2068 | 2068 | GCUACCUUCUCUCCAAUGA | 8422 | UCAUUGGAGAGAAGGUAG |
| | | | | C |
| siRNA 2069 | 2069 | CUACCUUCUCUCCAAUGAA | 8423 | UUCAUUGGAGAGAAGGUA |
| | | | | G |
| siRNA 2070 | 2070 | UACCUUCUCUCCAAUGAAG | 8424 | CUUCAUUGGAGAGAAGGU |
| | | | | A |
| siRNA 2071 | 2071 | ACCUUCUCUCCAAUGAAGG | 8425 | CCUUCAUUGGAGAGAAGG |
| | | | | U |
| siRNA 2072 | 2072 | CCUUCUCUCCAAUGAAGGC | 8426 | GCCUUCAUUGGAGAGAAG |
| | | | | G |
| siRNA 2073 | 2073 | CUUCUCUCCAAUGAAGGCA | 8427 | UGCCUUCAUUGGAGAGAA |
| | | | | G |
| siRNA 2074 | 2074 | UUCUCUCCAAUGAAGGCAC | 8428 | GUGCCUUCAUUGGAGAGA |
| | | | | A |
| siRNA 2075 | 2075 | UCUCUCCAAUGAAGGCACC | 8429 | GGUGCCUUCAUUGGAGAG |
| | | | | A |
| siRNA 2076 | 2076 | CUCUCCAAUGAAGGCACCA | 8430 | UGGUGCCUUCAUUGGAGA |
| | | | | G |
| siRNA 2077 | 2077 | UCUCCAAUGAAGGCACCAU | 8431 | AUGGUGCCUUCAUUGGAG |
| | | | | A |
| siRNA 2078 | 2078 | CUCCAAUGAAGGCACCAUC | 8432 | GAUGGUGCCUUCAUUGGA |
| | | | | G |
| siRNA 2079 | 2079 | UCCAAUGAAGGCACCAUCU | 8433 | AGAUGGUGCCUUCAUUGG |
| | | | | A |
| siRNA 2080 | 2080 | CCAAUGAAGGCACCAUCUC | 8434 | GAGAUGGUGCCUUCAUUG |
| | | | | G |
| siRNA 2081 | 2081 | CAAUGAAGGCACCAUCUCA | 8435 | UGAGAUGGUGCCUUCAUU |
| | | | | G |
| siRNA 2082 | 2082 | AAUGAAGGCACCAUCUCAC | 8436 | GUGAGAUGGUGCCUUCAU |
| | | | | U |
| siRNA 2083 | 2083 | AUGAAGGCACCAUCUCACU | 8437 | AGUGAGAUGGUGCCUUCA |
| | | | | U |
| siRNA 2084 | 2084 | UGAAGGCACCAUCUCACUG | 8438 | CAGUGAGAUGGUGCCUUCA |
| siRNA 2085 | 2085 | GAAGGCACCAUCUCACUGC | 8439 | GCAGUGAGAUGGUGCCUUC |
| siRNA 2086 | 2086 | AAGGCACCAUCUCACUGCC | 8440 | GGCAGUGAGAUGGUGCCU |
| | | | | U |
| siRNA 2087 | 2087 | AGGCACCAUCUCACUGCCU | 8441 | AGGCAGUGAGAUGGUGCC |
| | | | | U |
| siRNA 2088 | 2088 | GGCACCAUCUCACUGCCUU | 8442 | AAGGCAGUGAGAUGGUGC |
| | | | | C |
| siRNA 2089 | 2089 | GCACCAUCUCACUGCCUUG | 8443 | CAAGGCAGUGAGAUGGUG |
| | | | | C |
| siRNA 2090 | 2090 | CACCAUCUCACUGCCUUGC | 8444 | GCAAGGCAGUGAGAUGGU |
| | | | | G |
| siRNA 2091 | 2091 | ACCAUCUCACUGCCUUGCC | 8445 | GGCAAGGCAGUGAGAUGG |
| | | | | U |
| siRNA 2092 | 2092 | CCAUCUCACUGCCUUGCCA | 8446 | UGGCAAGGCAGUGAGAUG |
| | | | | G |
| siRNA 2093 | 2093 | CAUCUCACUGCCUUGCCAG | 8447 | CUGGCAAGGCAGUGAGAU |
| | | | | G |
| siRNA 2094 | 2094 | AUCUCACUGCCUUGCCAGA | 8448 | UCUGGCAAGGCAGUGAGA |
| | | | | U |
| siRNA 2095 | 2095 | UCUCACUGCCUUGCCAGAC | 8449 | GUCUGGCAAGGCAGUGAG |
| | | | | A |
| siRNA 2096 | 2096 | CUCACUGCCUUGCCAGACA | 8450 | UGUCUGGCAAGGCAGUGA |
| | | | | G |
| siRNA 2097 | 2097 | UCACUGCCUUGCCAGACAU | 8451 | AUGUCUGGCAAGGCAGUG |
| | | | | A |
| siRNA 2098 | 2098 | CACUGCCUUGCCAGACAUU | 8452 | AAUGUCUGGCAAGGCAGU |
| | | | | G |
| siRNA 2099 | 2099 | ACUGCCUUGCCAGACAUUU | 8453 | AAAUGUCUGGCAAGGCAG |
| | | | | U |
| siRNA 2100 | 2100 | CUGCCUUGCCAGACAUUUU | 8454 | AAAAUGUCUGGCAAGGCA |
| | | | | G |
| siRNA 2101 | 2101 | UGCCUUGCCAGACAUUUUA | 8455 | UAAAAUGUCUGGCAAGGC |
| | | | | A |
| siRNA 2102 | 2102 | GCCUUGCCAGACAUUUUAC | 8456 | GUAAAAUGUCUGGCAAGG |
| | | | | C |
| siRNA 2103 | 2103 | CCUUGCCAGACAUUUUACC | 8457 | GGUAAAAUGUCUGGCAAG |
| | | | | G |
| siRNA 2104 | 2104 | CUUGCCAGACAUUUUACCA | 8458 | UGGUAAAAUGUCUGGCAA |
| | | | | G |
| siRNA 2105 | 2105 | UUGCCAGACAUUUUACCAA | 8459 | UUGGUAAAAUGUCUGGCA |
| | | | | A |
| siRNA 2106 | 2106 | UGCCAGACAUUUUACCAAG | 8460 | CUUGGUAAAAUGUCUGGC |
| | | | | A |
| siRNA 2107 | 2107 | GCCAGACAUUUUACCAAGU | 8461 | ACUUGGUAAAAUGUCUGG |
| | | | | C |
| siRNA 2108 | 2108 | CCAGACAUUUUACCAAGUC | 8462 | GACUUGGUAAAAUGUCUG |
| | | | | G |
| siRNA 2109 | 2109 | CAGACAUUUUACCAAGUCU | 8463 | AGACUUGGUAAAAUGUCU |
| | | | | G |
| siRNA 2110 | 2110 | AGACAUUUUACCAAGUCUG | 8464 | CAGACUUGGUAAAAUGUC |
| | | | | U |
| siRNA 2111 | 2111 | GACAUUUUACCAAGUCUGC | 8465 | GCAGACUUGGUAAAAUGU |
| | | | | C |
| siRNA 2112 | 2112 | ACAUUUUACCAAGUCUGCC | 8466 | GGCAGACUUGGUAAAAUG |
| | | | | U |
| siRNA 2113 | 2113 | CAUUUUACCAAGUCUGCCA | 8467 | UGGCAGACUUGGUAAAAU |
| | | | | G |
| siRNA 2114 | 2114 | AUUUUACCAAGUCUGCCAU | 8468 | AUGGCAGACUUGGUAAAA |
| | | | | U |
| siRNA 2115 | 2115 | UUUUACCAAGUCUGCCAUG | 8469 | CAUGGCAGACUUGGUAAA |
| | | | | A |
| siRNA 2116 | 2116 | UUUACCAAGUCUGCCAUGA | 8470 | UCAUGGCAGACUUGGUAA |
| | | | | A |
| siRNA 2117 | 2117 | UUACCAAGUCUGCCAUGAA | 8471 | UUCAUGGCAGACUUGGUA |
| | | | | A |
| siRNA 2118 | 2118 | UACCAAGUCUGCCAUGAAA | 8472 | UUUCAUGGCAGACUUGGU |
| | | | | A |
| siRNA 2119 | 2119 | ACCAAGUCUGCCAUGAAAC | 8473 | GUUUCAUGGCAGACUUGG |
| | | | | U |
| siRNA 2120 | 2120 | CCAAGUCUGCCAUGAAACA | 8474 | UGUUUCAUGGCAGACUUG |
| | | | | G |
| siRNA 2121 | 2121 | CAAGUCUGCCAUGAAACAG | 8475 | CUGUUUCAUGGCAGACUUG |
| siRNA 2122 | 2122 | AAGUCUGCCAUGAAACAGU | 8476 | ACUGUUUCAUGGCAGACUU |
| siRNA 2123 | 2123 | AGUCUGCCAUGAAACAGUA | 8477 | UACUGUUUCAUGGCAGACU |
| siRNA 2124 | 2124 | GUCUGCCAUGAAACAGUAG | 8478 | CUACUGUUUCAUGGCAGAC |
| siRNA 2125 | 2125 | UCUGCCAUGAAACAGUAGG | 8479 | CCUACUGUUUCAUGGCAGA |
| siRNA 2126 | 2126 | CUGCCAUGAAACAGUAGGA | 8480 | UCCUACUGUUUCAUGGCAG |
| siRNA 2127 | 2127 | UGCCAUGAAACAGUAGGA | 8481 | UUCCUACUGUUUCAUGGCA |
| | | A | | |
| siRNA 2128 | 2128 | GCCAUGAAACAGUAGGAA | 8482 | UUUCCUACUGUUUCAUGGC |
| | | A | | |
| siRNA 2129 | 2129 | CCAUGAAACAGUAGGAAA | 8483 | CUUUCCUACUGUUUCAUGG |
| | | G | | |
| siRNA 2130 | 2130 | CAUGAAACAGUAGGAAAG | 8484 | ACUUUCCUACUGUUUCAUG |
| | | U | | |
| siRNA 2131 | 2131 | AUGAAACAGUAGGAAAGU | 8485 | AACUUUCCUACUGUUUCAU |
| | | U | | |
| siRNA 2132 | 2132 | UGAAACAGUAGGAAAGUU | 8486 | AAACUUUCCUACUGUUUCA |
| | | U | | |
| siRNA 2133 | 2133 | GAAACAGUAGGAAAGUUU | 8487 | UAAACUUUCCUACUGUUUC |
| | | A | | |
| siRNA 2134 | 2134 | AAACAGUAGGAAAGUUUA | 8488 | AUAAACUUUCCUACUGUUU |
| | | U | | |
| siRNA 2135 | 2135 | AACAGUAGGAAAGUUUAU | 8489 | GAUAAACUUUCCUACUGUU |
| | | C | | |
| siRNA 2136 | 2136 | ACAGUAGGAAAGUUUAUC | 8490 | GGAUAAACUUUCCUACUGU |
| | | C | | |
| siRNA 2137 | 2137 | CAGUAGGAAAGUUUAUCC | 8491 | UGGAUAAACUUUCCUACUG |
| | | A | | |
| siRNA 2138 | 2138 | AGUAGGAAAGUUUAUCCA | 8492 | CUGGAUAAACUUUCCUACU |
| | | G | | |
| siRNA 2139 | 2139 | GUAGGAAAGUUUAUCCAG | 8493 | ACUGGAUAAACUUUCCUAC |
| | | U | | |
| siRNA 2140 | 2140 | UAGGAAAGUUUAUCCAGU | 8494 | UACUGGAUAAACUUUCCUA |
| | | A | | |
| siRNA 2141 | 2141 | AGGAAAGUUUAUCCAGUA | 8495 | AUACUGGAUAAACUUUCCU |
| | | U | | |
| siRNA 2142 | 2142 | GGAAAGUUUAUCCAGUAU | 8496 | CAUACUGGAUAAACUUUCC |
| | | G | | |
| siRNA 2143 | 2143 | GAAAGUUUAUCCAGUAUG | 8497 | CCAUACUGGAUAAACUUUC |
| | | G | | |
| siRNA 2144 | 2144 | AAAGUUUAUCCAGUAUGG | 8498 | GCCAUACUGGAUAAACUUU |
| | | C | | |
| siRNA 2145 | 2145 | AAGUUUAUCCAGUAUGGC | 8499 | UGCCAUACUGGAUAAACUU |
| | | A | | |
| siRNA 2146 | 2146 | AGUUUAUCCAGUAUGGCA | 8500 | AUGCCAUACUGGAUAAACU |
| | | U | | |
| siRNA 2147 | 2147 | GUUUAUCCAGUAUGGCAU | 8501 | AAUGCCAUACUGGAUAAAC |
| | | U | | |
| siRNA 2148 | 2148 | UUUAUCCAGUAUGGCAUUC | 8502 | GAAUGCCAUACUGGAUAA |
| | | | | A |
| siRNA 2149 | 2149 | UUAUCCAGUAUGGCAUUCU | 8503 | AGAAUGCCAUACUGGAUA |
| | | | | A |
| siRNA 2150 | 2150 | UAUCCAGUAUGGCAUUCUU | 8504 | AAGAAUGCCAUACUGGAU |
| | | | | A |
| siRNA 2151 | 2151 | AUCCAGUAUGGCAUUCUUA | 8505 | UAAGAAUGCCAUACUGGA |
| | | | | U |
| siRNA 2152 | 2152 | UCCAGUAUGGCAUUCUUAC | 8506 | GUAAGAAUGCCAUACUGG |
| | | | | A |
| siRNA 2153 | 2153 | CCAGUAUGGCAUUCUUACA | 8507 | UGUAAGAAUGCCAUACUG |
| | | | | G |
| siRNA 2154 | 2154 | CAGUAUGGCAUUCUUACAG | 8508 | CUGUAAGAAUGCCAUACUG |
| siRNA 2155 | 2155 | AGUAUGGCAUUCUUACAG | 8509 | ACUGUAAGAAUGCCAUACU |
| | | U | | |
| siRNA 2156 | 2156 | GUAUGGCAUUCUUACAGU | 8510 | CACUGUAAGAAUGCCAUAC |
| | | G | | |
| siRNA 2157 | 2157 | UAUGGCAUUCUUACAGUG | 8511 | CCACUGUAAGAAUGCCAUA |
| | | G | | |
| siRNA 2158 | 2158 | AUGGCAUUCUUACAGUGGC | 8512 | GCCACUGUAAGAAUGCCAU |
| siRNA 2159 | 2159 | UGGCAUUCUUACAGUGGCA | 8513 | UGCCACUGUAAGAAUGCCA |
| siRNA 2160 | 2160 | GGCAUUCUUACAGUGGCAG | 8514 | CUGCCACUGUAAGAAUGCC |
| siRNA 2161 | 2161 | GCAUUCUUACAGUGGCAGA | 8515 | UCUGCCACUGUAAGAAUGC |
| siRNA 2162 | 2162 | CAUUCUUACAGUGGCAGAG | 8516 | CUCUGCCACUGUAAGAAUG |
| siRNA 2163 | 2163 | AUUCUUACAGUGGCAGAGC | 8517 | GCUCUGCCACUGUAAGAAU |
| siRNA 2164 | 2164 | UUCUUACAGUGGCAGAGCA | 8518 | UGCUCUGCCACUGUAAGAA |
| siRNA 2165 | 2165 | UCUUACAGUGGCAGAGCAC | 8519 | GUGCUCUGCCACUGUAAGA |
| siRNA 2166 | 2166 | CUUACAGUGGCAGAGCACG | 8520 | CGUGCUCUGCCACUGUAAG |
| siRNA 2167 | 2167 | UUACAGUGGCAGAGCACGA | 8521 | UCGUGCUCUGCCACUGUAA |
| siRNA 2168 | 2168 | UACAGUGGCAGAGCACGAU | 8522 | AUCGUGCUCUGCCACUGUA |
| siRNA 2169 | 2169 | ACAGUGGCAGAGCACGAUG | 8523 | CAUCGUGCUCUGCCACUGU |
| siRNA 2170 | 2170 | CAGUGGCAGAGCACGAUGA | 8524 | UCAUCGUGCUCUGCCACUG |
| siRNA 2171 | 2171 | AGUGGCAGAGCACGAUGAC | 8525 | GUCAUCGUGCUCUGCCACU |
| siRNA 2172 | 2172 | GUGGCAGAGCACGAUGACC | 8526 | GGUCAUCGUGCUCUGCCAC |
| siRNA 2173 | 2173 | UGGCAGAGCACGAUGACCA | 8527 | UGGUCAUCGUGCUCUGCCA |
| siRNA 2174 | 2174 | GGCAGAGCACGAUGACCAG | 8528 | CUGGUCAUCGUGCUCUGCC |
| siRNA 2175 | 2175 | GCAGAGCACGAUGACCAGG | 8529 | CCUGGUCAUCGUGCUCUGC |
| siRNA 2176 | 2176 | CAGAGCACGAUGACCAGGA | 8530 | UCCUGGUCAUCGUGCUCUG |
| siRNA 2177 | 2177 | AGAGCACGAUGACCAGGAA | 8531 | UUCCUGGUCAUCGUGCUCU |
| siRNA 2178 | 2178 | GAGCACGAUGACCAGGAAG | 8532 | CUUCCUGGUCAUCGUGCUC |
| siRNA 2179 | 2179 | AGCACGAUGACCAGGAAGA | 8533 | UCUUCCUGGUCAUCGUGCU |
| siRNA 2180 | 2180 | GCACGAUGACCAGGAAGAU | 8534 | AUCUUCCUGGUCAUCGUGC |
| siRNA 2181 | 2181 | CACGAUGACCAGGAAGAUA | 8535 | UAUCUUCCUGGUCAUCGUG |
| siRNA 2182 | 2182 | ACGAUGACCAGGAAGAUA | 8536 | AUAUCUUCCUGGUCAUCGU |
| | | U | | |
| siRNA 2183 | 2183 | CGAUGACCAGGAAGAUAUC | 8537 | GAUAUCUUCCUGGUCAUCG |
| siRNA 2184 | 2184 | GAUGACCAGGAAGAUAUC | 8538 | UGAUAUCUUCCUGGUCAUC |
| | | A | | |
| siRNA 2185 | 2185 | AUGACCAGGAAGAUAUCA | 8539 | CUGAUAUCUUCCUGGUCAU |
| | | G | | |
| siRNA 2186 | 2186 | UGACCAGGAAGAUAUCAG | 8540 | ACUGAUAUCUUCCUGGUCA |
| | | U | | |
| siRNA 2187 | 2187 | GACCAGGAAGAUAUCAGUC | 8541 | GACUGAUAUCUUCCUGGUC |
| siRNA 2188 | 2188 | ACCAGGAAGAUAUCAGUCC | 8542 | GGACUGAUAUCUUCCUGGU |
| siRNA 2189 | 2189 | CCAGGAAGAUAUCAGUCCU | 8543 | AGGACUGAUAUCUUCCUGG |
| siRNA 2190 | 2190 | CAGGAAGAUAUCAGUCCUA | 8544 | UAGGACUGAUAUCUUCCUG |
| siRNA 2191 | 2191 | AGGAAGAUAUCAGUCCUA | 8545 | CUAGGACUGAUAUCUUCCU |
| | | G | | |
| siRNA 2192 | 2192 | GGAAGAUAUCAGUCCUAG | 8546 | ACUAGGACUGAUAUCUUCC |
| | | U | | |
| siRNA 2193 | 2193 | GAAGAUAUCAGUCCUAGUC | 8547 | GACUAGGACUGAUAUCUUC |
| siRNA 2194 | 2194 | AAGAUAUCAGUCCUAGUCU | 8548 | AGACUAGGACUGAUAUCU |
| | | | | U |
| siRNA 2195 | 2195 | AGAUAUCAGUCCUAGUCUU | 8549 | AAGACUAGGACUGAUAUC |
| | | | | U |
| siRNA 2196 | 2196 | GAUAUCAGUCCUAGUCUUG | 8550 | CAAGACUAGGACUGAUAUC |
| siRNA 2197 | 2197 | AUAUCAGUCCUAGUCUUGC | 8551 | GCAAGACUAGGACUGAUA |
| | | | | U |
| siRNA 2198 | 2198 | UAUCAGUCCUAGUCUUGCU | 8552 | AGCAAGACUAGGACUGAU |
| | | | | A |
| siRNA 2199 | 2199 | AUCAGUCCUAGUCUUGCUG | 8553 | CAGCAAGACUAGGACUGAU |
| siRNA 2200 | 2200 | UCAGUCCUAGUCUUGCUGA | 8554 | UCAGCAAGACUAGGACUGA |
| siRNA 2201 | 2201 | CAGUCCUAGUCUUGCUGAG | 8555 | CUCAGCAAGACUAGGACUG |
| siRNA 2202 | 2202 | AGUCCUAGUCUUGCUGAGC | 8556 | GCUCAGCAAGACUAGGACU |
| siRNA 2203 | 2203 | GUCCUAGUCUUGCUGAGCA | 8557 | UGCUCAGCAAGACUAGGAC |
| siRNA 2204 | 2204 | UCCUAGUCUUGCUGAGCAG | 8558 | CUGCUCAGCAAGACUAGGA |
| siRNA 2205 | 2205 | CCUAGUCUUGCUGAGCAGC | 8559 | GCUGCUCAGCAAGACUAGG |
| siRNA 2206 | 2206 | CUAGUCUUGCUGAGCAGCA | 8560 | UGCUGCUCAGCAAGACUAG |
| siRNA 2207 | 2207 | UAGUCUUGCUGAGCAGCAG | 8561 | CUGCUGCUCAGCAAGACUA |
| siRNA 2208 | 2208 | AGUCUUGCUGAGCAGCAGU | 8562 | ACUGCUGCUCAGCAAGACU |
| siRNA 2209 | 2209 | GUCUUGCUGAGCAGCAGUG | 8563 | CACUGCUGCUCAGCAAGAC |
| siRNA 2210 | 2210 | UCUUGCUGAGCAGCAGUGG | 8564 | CCACUGCUGCUCAGCAAGA |
| siRNA 2211 | 2211 | CUUGCUGAGCAGCAGUGGG | 8565 | CCCACUGCUGCUCAGCAAG |
| siRNA 2212 | 2212 | UUGCUGAGCAGCAGUGGG | 8566 | UCCCACUGCUGCUCAGCAA |
| | | A | | |
| siRNA 2213 | 2213 | UGCUGAGCAGCAGUGGGAC | 8567 | GUCCCACUGCUGCUCAGCA |
| siRNA 2214 | 2214 | GCUGAGCAGCAGUGGGACA | 8568 | UGUCCCACUGCUGCUCAGC |
| siRNA 2215 | 2215 | CUGAGCAGCAGUGGGACAA | 8569 | UUGUCCCACUGCUGCUCAG |
| siRNA 2216 | 2216 | UGAGCAGCAGUGGGACAA | 8570 | CUUGUCCCACUGCUGCUCA |
| | | G | | |
| siRNA 2217 | 2217 | GAGCAGCAGUGGGACAAG | 8571 | UCUUGUCCCACUGCUGCUC |
| | | A | | |
| siRNA 2218 | 2218 | AGCAGCAGUGGGACAAGA | 8572 | UUCUUGUCCCACUGCUGCU |
| | | A | | |
| siRNA 2219 | 2219 | GCAGCAGUGGGACAAGAA | 8573 | CUUCUUGUCCCACUGCUGC |
| | | G | | |
| siRNA 2220 | 2220 | CAGCAGUGGGACAAGAAGC | 8574 | GCUUCUUGUCCCACUGCUG |
| siRNA 2221 | 2221 | AGCAGUGGGACAAGAAGC | 8575 | AGCUUCUUGUCCCACUGCU |
| | | U | | |
| siRNA 2222 | 2222 | GCAGUGGGACAAGAAGCU | 8576 | AAGCUUCUUGUCCCACUGC |
| | | U | | |
| siRNA 2223 | 2223 | CAGUGGGACAAGAAGCUUC | 8577 | GAAGCUUCUUGUCCCACUG |
| siRNA 2224 | 2224 | AGUGGGACAAGAAGCUUCC | 8578 | GGAAGCUUCUUGUCCCACU |
| siRNA 2225 | 2225 | GUGGGACAAGAAGCUUCCA | 8579 | UGGAAGCUUCUUGUCCCAC |
| siRNA 2226 | 2226 | UGGGACAAGAAGCUUCCAG | 8580 | CUGGAAGCUUCUUGUCCCA |
| siRNA 2227 | 2227 | GGGACAAGAAGCUUCCAGA | 8581 | UCUGGAAGCUUCUUGUCCC |
| siRNA 2228 | 2228 | GGACAAGAAGCUUCCAGAA | 8582 | UUCUGGAAGCUUCUUGUCC |
| siRNA 2229 | 2229 | GACAAGAAGCUUCCAGAAC | 8583 | GUUCUGGAAGCUUCUUGUC |
| siRNA 2230 | 2230 | ACAAGAAGCUUCCAGAACC | 8584 | GGUUCUGGAAGCUUCUUG |
| | | | | U |
| siRNA 2231 | 2231 | CAAGAAGCUUCCAGAACCU | 8585 | AGGUUCUGGAAGCUUCUU |
| | | | | G |
| siRNA 2232 | 2232 | AAGAAGCUUCCAGAACCUU | 8586 | AAGGUUCUGGAAGCUUCU |
| | | | | U |
| siRNA 2233 | 2233 | AGAAGCUUCCAGAACCUUU | 8587 | AAAGGUUCUGGAAGCUUC |
| | | | | U |
| siRNA 2234 | 2234 | GAAGCUUCCAGAACCUUUG | 8588 | CAAAGGUUCUGGAAGCUUC |
| siRNA 2235 | 2235 | AAGCUUCCAGAACCUUUGU | 8589 | ACAAAGGUUCUGGAAGCU |
| | | | | U |
| siRNA 2236 | 2236 | AGCUUCCAGAACCUUUGUC | 8590 | GACAAAGGUUCUGGAAGC |
| | | | | U |
| siRNA 2237 | 2237 | GCUUCCAGAACCUUUGUCU | 8591 | AGACAAAGGUUCUGGAAG |
| | | | | C |
| siRNA 2238 | 2238 | CUUCCAGAACCUUUGUCUU | 8592 | AAGACAAAGGUUCUGGAA |
| | | | | G |
| siRNA 2239 | 2239 | UUCCAGAACCUUUGUCUUG | 8593 | CAAGACAAAGGUUCUGGA |
| | | | | A |
| siRNA 2240 | 2240 | UCCAGAACCUUUGUCUUGG | 8594 | CCAAGACAAAGGUUCUGGA |
| siRNA 2241 | 2241 | CCAGAACCUUUGUCUUGGA | 8595 | UCCAAGACAAAGGUUCUGG |
| siRNA 2242 | 2242 | CAGAACCUUUGUCUUGGAG | 8596 | CUCCAAGACAAAGGUUCUG |
| siRNA 2243 | 2243 | AGAACCUUUGUCUUGGAG | 8597 | UCUCCAAGACAAAGGUUCU |
| | | A | | |
| siRNA 2244 | 2244 | GAACCUUUGUCUUGGAGA | 8598 | UUCUCCAAGACAAAGGUUC |
| | | A | | |
| siRNA 2245 | 2245 | AACCUUUGUCUUGGAGAA | 8599 | CUUCUCCAAGACAAAGGUU |
| | | G | | |
| siRNA 2246 | 2246 | ACCUUUGUCUUGGAGAAG | 8600 | ACUUCUCCAAGACAAAGGU |
| | | U | | |
| siRNA 2247 | 2247 | CCUUUGUCUUGGAGAAGU | 8601 | CACUUCUCCAAGACAAAGG |
| | | G | | |
| siRNA 2248 | 2248 | CUUUGUCUUGGAGAAGUG | 8602 | UCACUUCUCCAAGACAAAG |
| | | A | | |
| siRNA 2249 | 2249 | UUUGUCUUGGAGAAGUGA | 8603 | AUCACUUCUCCAAGACAAA |
| | | U | | |
| siRNA 2250 | 2250 | UUGUCUUGGAGAAGUGAU | 8604 | CAUCACUUCUCCAAGACAA |
| | | G | | |
| siRNA 2251 | 2251 | UGUCUUGGAGAAGUGAUG | 8605 | UCAUCACUUCUCCAAGACA |
| | | A | | |
| siRNA 2252 | 2252 | GUCUUGGAGAAGUGAUGA | 8606 | UUCAUCACUUCUCCAAGAC |
| | | A | | |
| siRNA 2253 | 2253 | UCUUGGAGAAGUGAUGAA | 8607 | CUUCAUCACUUCUCCAAGA |
| | | G | | |
| siRNA 2254 | 2254 | CUUGGAGAAGUGAUGAAG | 8608 | UCUUCAUCACUUCUCCAAG |
| | | A | | |
| siRNA 2255 | 2255 | UUGGAGAAGUGAUGAAGA | 8609 | UUCUUCAUCACUUCUCCAA |
| | | A | | |
| siRNA 2256 | 2256 | UGGAGAAGUGAUGAAGAA | 8610 | CUUCUUCAUCACUUCUCCA |
| | | G | | |
| siRNA 2257 | 2257 | GGAGAAGUGAUGAAGAAG | 8611 | UCUUCUUCAUCACUUCUCC |
| | | A | | |
| siRNA 2258 | 2258 | GAGAAGUGAUGAAGAAGA | 8612 | AUCUUCUUCAUCACUUCUC |
| | | U | | |
| siRNA 2259 | 2259 | AGAAGUGAUGAAGAAGAU | 8613 | CAUCUUCUUCAUCACUUCU |
| | | G | | |
| siRNA 2260 | 2260 | GAAGUGAUGAAGAAGAUG | 8614 | UCAUCUUCUUCAUCACUUC |
| | | A | | |
| siRNA 2261 | 2261 | AAGUGAUGAAGAAGAUGA | 8615 | UUCAUCUUCUUCAUCACUU |
| | | A | | |
| siRNA 2262 | 2262 | AGUGAUGAAGAAGAUGAA | 8616 | CUUCAUCUUCUUCAUCACU |
| | | G | | |
| siRNA 2263 | 2263 | GUGAUGAAGAAGAUGAAG | 8617 | UCUUCAUCUUCUUCAUCAC |
| | | A | | |
| siRNA 2264 | 2264 | UGAUGAAGAAGAUGAAGA | 8618 | GUCUUCAUCUUCUUCAUCA |
| | | C | | |
| siRNA 2265 | 2265 | GAUGAAGAAGAUGAAGAC | 8619 | UGUCUUCAUCUUCUUCAUC |
| | | A | | |
| siRNA 2266 | 2266 | AUGAAGAAGAUGAAGACA | 8620 | CUGUCUUCAUCUUCUUCAU |
| | | G | | |
| siRNA 2267 | 2267 | UGAAGAAGAUGAAGACAG | 8621 | ACUGUCUUCAUCUUCUUCA |
| | | U | | |
| siRNA 2268 | 2268 | GAAGAAGAUGAAGACAGU | 8622 | CACUGUCUUCAUCUUCUUC |
| | | G | | |
| siRNA 2269 | 2269 | AAGAAGAUGAAGACAGUG | 8623 | UCACUGUCUUCAUCUUCUU |
| | | A | | |
| siRNA 2270 | 2270 | AGAAGAUGAAGACAGUGA | 8624 | GUCACUGUCUUCAUCUUCU |
| | | C | | |
| siRNA 2271 | 2271 | GAAGAUGAAGACAGUGAC | 8625 | AGUCACUGUCUUCAUCUUC |
| | | U | | |
| siRNA 2272 | 2272 | AAGAUGAAGACAGUGACU | 8626 | AAGUCACUGUCUUCAUCUU |
| | | U | | |
| siRNA 2273 | 2273 | AGAUGAAGACAGUGACUU | 8627 | AAAGUCACUGUCUUCAUCU |
| | | U | | |
| siRNA 2274 | 2274 | GAUGAAGACAGUGACUUU | 8628 | CAAAGUCACUGUCUUCAUC |
| | | G | | |
| siRNA 2275 | 2275 | AUGAAGACAGUGACUUUG | 8629 | CCAAAGUCACUGUCUUCAU |
| | | G | | |
| siRNA 2276 | 2276 | UGAAGACAGUGACUUUGG | 8630 | CCCAAAGUCACUGUCUUCA |
| | | G | | |
| siRNA 2277 | 2277 | GAAGACAGUGACUUUGGG | 8631 | CCCCAAAGUCACUGUCUUC |
| | | G | | |
| siRNA 2278 | 2278 | AAGACAGUGACUUUGGGG | 8632 | UCCCCAAAGUCACUGUCUU |
| | | A | | |
| siRNA 2279 | 2279 | AGACAGUGACUUUGGGGA | 8633 | CUCCCCAAAGUCACUGUCU |
| | | G | | |
| siRNA 2280 | 2280 | GACAGUGACUUUGGGGAG | 8634 | CCUCCCCAAAGUCACUGUC |
| | | G | | |
| siRNA 2281 | 2281 | ACAGUGACUUUGGGGAGG | 8635 | UCCUCCCCAAAGUCACUGU |
| | | A | | |
| siRNA 2282 | 2282 | CAGUGACUUUGGGGAGGA | 8636 | UUCCUCCCCAAAGUCACUG |
| | | A | | |
| siRNA 2283 | 2283 | AGUGACUUUGGGGAGGAA | 8637 | GUUCCUCCCCAAAGUCACU |
| | | C | | |
| siRNA 2284 | 2284 | GUGACUUUGGGGAGGAAC | 8638 | UGUUCCUCCCCAAAGUCAC |
| | | A | | |
| siRNA 2285 | 2285 | UGACUUUGGGGAGGAACA | 8639 | CUGUUCCUCCCCAAAGUCA |
| | | G | | |
| siRNA 2286 | 2286 | GACUUUGGGGAGGAACAG | 8640 | GCUGUUCCUCCCCAAAGUC |
| | | C | | |
| siRNA 2287 | 2287 | ACUUUGGGGAGGAACAGC | 8641 | CGCUGUUCCUCCCCAAAGU |
| | | G | | |
| siRNA 2288 | 2288 | CUUUGGGGAGGAACAGCG | 8642 | UCGCUGUUCCUCCCCAAAG |
| | | A | | |
| siRNA 2289 | 2289 | UUUGGGGAGGAACAGCGA | 8643 | CUCGCUGUUCCUCCCCAAA |
| | | G | | |
| siRNA 2290 | 2290 | UUGGGGAGGAACAGCGAG | 8644 | UCUCGCUGUUCCUCCCCAA |
| | | A | | |
| siRNA 2291 | 2291 | UGGGGAGGAACAGCGAGA | 8645 | AUCUCGCUGUUCCUCCCCA |
| | | U | | |
| siRNA 2292 | 2292 | GGGGAGGAACAGCGAGAU | 8646 | AAUCUCGCUGUUCCUCCCC |
| | | U | | |
| siRNA 2293 | 2293 | GGGAGGAACAGCGAGAUU | 8647 | CAAUCUCGCUGUUCCUCCC |
| | | G | | |
| siRNA 2294 | 2294 | GGAGGAACAGCGAGAUUG | 8648 | GCAAUCUCGCUGUUCCUCC |
| | | C | | |
| siRNA 2295 | 2295 | GAGGAACAGCGAGAUUGC | 8649 | AGCAAUCUCGCUGUUCCUC |
| | | U | | |
| siRNA 2296 | 2296 | AGGAACAGCGAGAUUGCU | 8650 | UAGCAAUCUCGCUGUUCCU |
| | | A | | |
| siRNA 2297 | 2297 | GGAACAGCGAGAUUGCUAC | 8651 | GUAGCAAUCUCGCUGUUCC |
| siRNA 2298 | 2298 | GAACAGCGAGAUUGCUACC | 8652 | GGUAGCAAUCUCGCUGUUC |
| siRNA 2299 | 2299 | AACAGCGAGAUUGCUACCU | 8653 | AGGUAGCAAUCUCGCUGUU |
| siRNA 2300 | 2300 | ACAGCGAGAUUGCUACCUG | 8654 | CAGGUAGCAAUCUCGCUGU |
| siRNA 2301 | 2301 | CAGCGAGAUUGCUACCUGA | 8655 | UCAGGUAGCAAUCUCGCUG |
| siRNA 2302 | 2302 | AGCGAGAUUGCUACCUGAA | 8656 | UUCAGGUAGCAAUCUCGCU |
| siRNA 2303 | 2303 | GCGAGAUUGCUACCUGAAG | 8657 | CUUCAGGUAGCAAUCUCGC |
| siRNA 2304 | 2304 | CGAGAUUGCUACCUGAAGG | 8658 | CCUUCAGGUAGCAAUCUCG |
| siRNA 2305 | 2305 | GAGAUUGCUACCUGAAGG | 8659 | ACCUUCAGGUAGCAAUCUC |
| | | U | | |
| siRNA 2306 | 2306 | AGAUUGCUACCUGAAGGU | 8660 | CACCUUCAGGUAGCAAUCU |
| | | G | | |
| siRNA 2307 | 2307 | GAUUGCUACCUGAAGGUG | 8661 | UCACCUUCAGGUAGCAAUC |
| | | A | | |
| siRNA 2308 | 2308 | AUUGCUACCUGAAGGUGA | 8662 | CUCACCUUCAGGUAGCAAU |
| | | G | | |
| siRNA 2309 | 2309 | UUGCUACCUGAAGGUGAGC | 8663 | GCUCACCUUCAGGUAGCAA |
| siRNA 2310 | 2310 | UGCUACCUGAAGGUGAGCC | 8664 | GGCUCACCUUCAGGUAGCA |
| siRNA 2311 | 2311 | GCUACCUGAAGGUGAGCCA | 8665 | UGGCUCACCUUCAGGUAGC |
| siRNA 2312 | 2312 | CUACCUGAAGGUGAGCCAA | 8666 | UUGGCUCACCUUCAGGUAG |
| siRNA 2313 | 2313 | UACCUGAAGGUGAGCCAAU | 8667 | AUUGGCUCACCUUCAGGUA |
| siRNA 2314 | 2314 | ACCUGAAGGUGAGCCAAUC | 8668 | GAUUGGCUCACCUUCAGGU |
| siRNA 2315 | 2315 | CCUGAAGGUGAGCCAAUCC | 8669 | GGAUUGGCUCACCUUCAGG |
| siRNA 2316 | 2316 | CUGAAGGUGAGCCAAUCCA | 8670 | UGGAUUGGCUCACCUUCAG |
| siRNA 2317 | 2317 | UGAAGGUGAGCCAAUCCAA | 8671 | UUGGAUUGGCUCACCUUCA |
| siRNA 2318 | 2318 | GAAGGUGAGCCAAUCCAAG | 8672 | CUUGGAUUGGCUCACCUUC |
| siRNA 2319 | 2319 | AAGGUGAGCCAAUCCAAGG | 8673 | CCUUGGAUUGGCUCACCUU |
| siRNA 2320 | 2320 | AGGUGAGCCAAUCCAAGGA | 8674 | UCCUUGGAUUGGCUCACCU |
| siRNA 2321 | 2321 | GGUGAGCCAAUCCAAGGAG | 8675 | CUCCUUGGAUUGGCUCACC |
| siRNA 2322 | 2322 | GUGAGCCAAUCCAAGGAGC | 8676 | GCUCCUUGGAUUGGCUCAC |
| siRNA 2323 | 2323 | UGAGCCAAUCCAAGGAGCA | 8677 | UGCUCCUUGGAUUGGCUCA |
| siRNA 2324 | 2324 | GAGCCAAUCCAAGGAGCAC | 8678 | GUGCUCCUUGGAUUGGCUC |
| siRNA 2325 | 2325 | AGCCAAUCCAAGGAGCACC | 8679 | GGUGCUCCUUGGAUUGGCU |
| siRNA 2326 | 2326 | GCCAAUCCAAGGAGCACCA | 8680 | UGGUGCUCCUUGGAUUGGC |
| siRNA 2327 | 2327 | CCAAUCCAAGGAGCACCAG | 8681 | CUGGUGCUCCUUGGAUUGG |
| siRNA 2328 | 2328 | CAAUCCAAGGAGCACCAGC | 8682 | GCUGGUGCUCCUUGGAUUG |
| siRNA 2329 | 2329 | AAUCCAAGGAGCACCAGCA | 8683 | UGCUGGUGCUCCUUGGAUU |
| siRNA 2330 | 2330 | AUCCAAGGAGCACCAGCAG | 8684 | CUGCUGGUGCUCCUUGGAU |
| siRNA 2331 | 2331 | UCCAAGGAGCACCAGCAGU | 8685 | ACUGCUGGUGCUCCUUGGA |
| siRNA 2332 | 2332 | CCAAGGAGCACCAGCAGUU | 8686 | AACUGCUGGUGCUCCUUGG |
| siRNA 2333 | 2333 | CAAGGAGCACCAGCAGUUU | 8687 | AAACUGCUGGUGCUCCUUG |
| siRNA 2334 | 2334 | AAGGAGCACCAGCAGUUUA | 8688 | UAAACUGCUGGUGCUCCUU |
| siRNA 2335 | 2335 | AGGAGCACCAGCAGUUUAU | 8689 | AUAAACUGCUGGUGCUCCU |
| siRNA 2336 | 2336 | GGAGCACCAGCAGUUUAUC | 8690 | GAUAAACUGCUGGUGCUCC |
| siRNA 2337 | 2337 | GAGCACCAGCAGUUUAUCA | 8691 | UGAUAAACUGCUGGUGCUC |
| siRNA 2338 | 2338 | AGCACCAGCAGUUUAUCAC | 8692 | GUGAUAAACUGCUGGUGC |
| | | | | U |
| siRNA 2339 | 2339 | GCACCAGCAGUUUAUCACC | 8693 | GGUGAUAAACUGCUGGUG |
| | | | | C |
| siRNA 2340 | 2340 | CACCAGCAGUUUAUCACCU | 8694 | AGGUGAUAAACUGCUGGU |
| | | | | G |
| siRNA 2341 | 2341 | ACCAGCAGUUUAUCACCUU | 8695 | AAGGUGAUAAACUGCUGG |
| | | | | U |
| siRNA 2342 | 2342 | CCAGCAGUUUAUCACCUUC | 8696 | GAAGGUGAUAAACUGCUG |
| | | | | G |
| siRNA 2343 | 2343 | CAGCAGUUUAUCACCUUCU | 8697 | AGAAGGUGAUAAACUGCU |
| | | | | G |
| siRNA 2344 | 2344 | AGCAGUUUAUCACCUUCUU | 8698 | AAGAAGGUGAUAAACUGC |
| | | | | U |
| siRNA 2345 | 2345 | GCAGUUUAUCACCUUCUUA | 8699 | UAAGAAGGUGAUAAACUG |
| | | | | C |
| siRNA 2346 | 2346 | CAGUUUAUCACCUUCUUAC | 8700 | GUAAGAAGGUGAUAAACU |
| | | | | G |
| siRNA 2347 | 2347 | AGUUUAUCACCUUCUUACA | 8701 | UGUAAGAAGGUGAUAAAC |
| | | | | U |
| siRNA 2348 | 2348 | GUUUAUCACCUUCUUACAG | 8702 | CUGUAAGAAGGUGAUAAA |
| | | | | C |
| siRNA 2349 | 2349 | UUUAUCACCUUCUUACAGA | 8703 | UCUGUAAGAAGGUGAUAA |
| | | | | A |
| siRNA 2350 | 2350 | UUAUCACCUUCUUACAGAG | 8704 | CUCUGUAAGAAGGUGAUA |
| | | | | A |
| siRNA 2351 | 2351 | UAUCACCUUCUUACAGAGA | 8705 | UCUCUGUAAGAAGGUGAU |
| | | | | A |
| siRNA 2352 | 2352 | AUCACCUUCUUACAGAGAC | 8706 | GUCUCUGUAAGAAGGUGA |
| | | | | U |
| siRNA 2353 | 2353 | UCACCUUCUUACAGAGACU | 8707 | AGUCUCUGUAAGAAGGUG |
| | | | | A |
| siRNA 2354 | 2354 | CACCUUCUUACAGAGACUC | 8708 | GAGUCUCUGUAAGAAGGU |
| | | | | G |
| siRNA 2355 | 2355 | ACCUUCUUACAGAGACUCC | 8709 | GGAGUCUCUGUAAGAAGG |
| | | | | U |
| siRNA 2356 | 2356 | CCUUCUUACAGAGACUCCU | 8710 | AGGAGUCUCUGUAAGAAG |
| | | | | G |
| siRNA 2357 | 2357 | CUUCUUACAGAGACUCCUU | 8711 | AAGGAGUCUCUGUAAGAA |
| | | | | G |
| siRNA 2358 | 2358 | UUCUUACAGAGACUCCUUG | 8712 | CAAGGAGUCUCUGUAAGA |
| | | | | A |
| siRNA 2359 | 2359 | UCUUACAGAGACUCCUUGG | 8713 | CCAAGGAGUCUCUGUAAGA |
| siRNA 2360 | 2360 | CUUACAGAGACUCCUUGGG | 8714 | CCCAAGGAGUCUCUGUAAG |
| siRNA 2361 | 2361 | UUACAGAGACUCCUUGGGC | 8715 | GCCCAAGGAGUCUCUGUAA |
| siRNA 2362 | 2362 | UACAGAGACUCCUUGGGCC | 8716 | GGCCCAAGGAGUCUCUGUA |
| siRNA 2363 | 2363 | ACAGAGACUCCUUGGGCCU | 8717 | AGGCCCAAGGAGUCUCUGU |
| siRNA 2364 | 2364 | CAGAGACUCCUUGGGCCUU | 8718 | AAGGCCCAAGGAGUCUCUG |
| siRNA 2365 | 2365 | AGAGACUCCUUGGGCCUUU | 8719 | AAAGGCCCAAGGAGUCUCU |
| siRNA 2366 | 2366 | GAGACUCCUUGGGCCUUUG | 8720 | CAAAGGCCCAAGGAGUCUC |
| siRNA 2367 | 2367 | AGACUCCUUGGGCCUUUGC | 8721 | GCAAAGGCCCAAGGAGUCU |
| siRNA 2368 | 2368 | GACUCCUUGGGCCUUUGCU | 8722 | AGCAAAGGCCCAAGGAGUC |
| siRNA 2369 | 2369 | ACUCCUUGGGCCUUUGCUG | 8723 | CAGCAAAGGCCCAAGGAGU |
| siRNA 2370 | 2370 | CUCCUUGGGCCUUUGCUGG | 8724 | CCAGCAAAGGCCCAAGGAG |
| siRNA 2371 | 2371 | UCCUUGGGCCUUUGCUGGA | 8725 | UCCAGCAAAGGCCCAAGGA |
| siRNA 2372 | 2372 | CCUUGGGCCUUUGCUGGAG | 8726 | CUCCAGCAAAGGCCCAAGG |
| siRNA 2373 | 2373 | CUUGGGCCUUUGCUGGAGG | 8727 | CCUCCAGCAAAGGCCCAAG |
| siRNA 2374 | 2374 | UUGGGCCUUUGCUGGAGGC | 8728 | GCCUCCAGCAAAGGCCCAA |
| siRNA 2375 | 2375 | UGGGCCUUUGCUGGAGGCC | 8729 | GGCCUCCAGCAAAGGCCCA |
| siRNA 2376 | 2376 | GGGCCUUUGCUGGAGGCCU | 8730 | AGGCCUCCAGCAAAGGCCC |
| siRNA 2377 | 2377 | GGCCUUUGCUGGAGGCCUA | 8731 | UAGGCCUCCAGCAAAGGCC |
| siRNA 2378 | 2378 | GCCUUUGCUGGAGGCCUAC | 8732 | GUAGGCCUCCAGCAAAGGC |
| siRNA 2379 | 2379 | CCUUUGCUGGAGGCCUACA | 8733 | UGUAGGCCUCCAGCAAAGG |
| siRNA 2380 | 2380 | CUUUGCUGGAGGCCUACAG | 8734 | CUGUAGGCCUCCAGCAAAG |
| siRNA 2381 | 2381 | UUUGCUGGAGGCCUACAGC | 8735 | GCUGUAGGCCUCCAGCAAA |
| siRNA 2382 | 2382 | UUGCUGGAGGCCUACAGCU | 8736 | AGCUGUAGGCCUCCAGCAA |
| siRNA 2383 | 2383 | UGCUGGAGGCCUACAGCUC | 8737 | GAGCUGUAGGCCUCCAGCA |
| siRNA 2384 | 2384 | GCUGGAGGCCUACAGCUCU | 8738 | AGAGCUGUAGGCCUCCAGC |
| siRNA 2385 | 2385 | CUGGAGGCCUACAGCUCUG | 8739 | CAGAGCUGUAGGCCUCCAG |
| siRNA 2386 | 2386 | UGGAGGCCUACAGCUCUGC | 8740 | GCAGAGCUGUAGGCCUCCA |
| siRNA 2387 | 2387 | GGAGGCCUACAGCUCUGCU | 8741 | AGCAGAGCUGUAGGCCUCC |
| siRNA 2388 | 2388 | GAGGCCUACAGCUCUGCUG | 8742 | CAGCAGAGCUGUAGGCCUC |
| siRNA 2389 | 2389 | AGGCCUACAGCUCUGCUGC | 8743 | GCAGCAGAGCUGUAGGCCU |
| siRNA 2390 | 2390 | GGCCUACAGCUCUGCUGCC | 8744 | GGCAGCAGAGCUGUAGGCC |
| siRNA 2391 | 2391 | GCCUACAGCUCUGCUGCCA | 8745 | UGGCAGCAGAGCUGUAGGC |
| siRNA 2392 | 2392 | CCUACAGCUCUGCUGCCAU | 8746 | AUGGCAGCAGAGCUGUAG |
| | | | | G |
| siRNA 2393 | 2393 | CUACAGCUCUGCUGCCAUC | 8747 | GAUGGCAGCAGAGCUGUA |
| | | | | G |
| siRNA 2394 | 2394 | UACAGCUCUGCUGCCAUCU | 8748 | AGAUGGCAGCAGAGCUGU |
| | | | | A |
| siRNA 2395 | 2395 | ACAGCUCUGCUGCCAUCUU | 8749 | AAGAUGGCAGCAGAGCUG |
| | | | | U |
| siRNA 2396 | 2396 | CAGCUCUGCUGCCAUCUUU | 8750 | AAAGAUGGCAGCAGAGCU |
| | | | | G |
| siRNA 2397 | 2397 | AGCUCUGCUGCCAUCUUUG | 8751 | CAAAGAUGGCAGCAGAGCU |
| siRNA 2398 | 2398 | GCUCUGCUGCCAUCUUUGU | 8752 | ACAAAGAUGGCAGCAGAGC |
| siRNA 2399 | 2399 | CUCUGCUGCCAUCUUUGUU | 8753 | AACAAAGAUGGCAGCAGA |
| | | | | G |
| siRNA 2400 | 2400 | UCUGCUGCCAUCUUUGUUC | 8754 | GAACAAAGAUGGCAGCAG |
| | | | | A |
| siRNA 2401 | 2401 | CUGCUGCCAUCUUUGUUCA | 8755 | UGAACAAAGAUGGCAGCA |
| | | | | G |
| siRNA 2402 | 2402 | UGCUGCCAUCUUUGUUCAC | 8756 | GUGAACAAAGAUGGCAGC |
| | | | | A |
| siRNA 2403 | 2403 | GCUGCCAUCUUUGUUCACA | 8757 | UGUGAACAAAGAUGGCAG |
| | | | | C |
| siRNA 2404 | 2404 | CUGCCAUCUUUGUUCACAA | 8758 | UUGUGAACAAAGAUGGCA |
| | | | | G |
| siRNA 2405 | 2405 | UGCCAUCUUUGUUCACAAC | 8759 | GUUGUGAACAAAGAUGGC |
| | | | | A |
| siRNA 2406 | 2406 | GCCAUCUUUGUUCACAACU | 8760 | AGUUGUGAACAAAGAUGG |
| | | | | C |
| siRNA 2407 | 2407 | CCAUCUUUGUUCACAACUU | 8761 | AAGUUGUGAACAAAGAUG |
| | | | | G |
| siRNA 2408 | 2408 | CAUCUUUGUUCACAACUUC | 8762 | GAAGUUGUGAACAAAGAU |
| | | | | G |
| siRNA 2409 | 2409 | AUCUUUGUUCACAACUUCA | 8763 | UGAAGUUGUGAACAAAGA |
| | | | | U |
| siRNA 2410 | 2410 | UCUUUGUUCACAACUUCAG | 8764 | CUGAAGUUGUGAACAAAG |
| | | | | A |
| siRNA 2411 | 2411 | CUUUGUUCACAACUUCAGU | 8765 | ACUGAAGUUGUGAACAAA |
| | | | | G |
| siRNA 2412 | 2412 | UUUGUUCACAACUUCAGUG | 8766 | CACUGAAGUUGUGAACAA |
| | | | | A |
| siRNA 2413 | 2413 | UUGUUCACAACUUCAGUGG | 8767 | CCACUGAAGUUGUGAACAA |
| siRNA 2414 | 2414 | UGUUCACAACUUCAGUGGU | 8768 | ACCACUGAAGUUGUGAACA |
| siRNA 2415 | 2415 | GUUCACAACUUCAGUGGUC | 8769 | GACCACUGAAGUUGUGAAC |
| siRNA 2416 | 2416 | UUCACAACUUCAGUGGUCC | 8770 | GGACCACUGAAGUUGUGA |
| | | | | A |
| siRNA 2417 | 2417 | UCACAACUUCAGUGGUCCU | 8771 | AGGACCACUGAAGUUGUG |
| | | | | A |
| siRNA 2418 | 2418 | CACAACUUCAGUGGUCCUG | 8772 | CAGGACCACUGAAGUUGUG |
| siRNA 2419 | 2419 | ACAACUUCAGUGGUCCUGU | 8773 | ACAGGACCACUGAAGUUGU |
| siRNA 2420 | 2420 | CAACUUCAGUGGUCCUGUU | 8774 | AACAGGACCACUGAAGUUG |
| siRNA 2421 | 2421 | AACUUCAGUGGUCCUGUUC | 8775 | GAACAGGACCACUGAAGUU |
| siRNA 2422 | 2422 | ACUUCAGUGGUCCUGUUCC | 8776 | GGAACAGGACCACUGAAGU |
| siRNA 2423 | 2423 | CUUCAGUGGUCCUGUUCCA | 8777 | UGGAACAGGACCACUGAAG |
| siRNA 2424 | 2424 | UUCAGUGGUCCUGUUCCAG | 8778 | CUGGAACAGGACCACUGAA |
| siRNA 2425 | 2425 | UCAGUGGUCCUGUUCCAGA | 8779 | UCUGGAACAGGACCACUGA |
| siRNA 2426 | 2426 | CAGUGGUCCUGUUCCAGAA | 8780 | UUCUGGAACAGGACCACUG |
| siRNA 2427 | 2427 | AGUGGUCCUGUUCCAGAAC | 8781 | GUUCUGGAACAGGACCACU |
| siRNA 2428 | 2428 | GUGGUCCUGUUCCAGAACC | 8782 | GGUUCUGGAACAGGACCAC |
| siRNA 2429 | 2429 | UGGUCCUGUUCCAGAACCU | 8783 | AGGUUCUGGAACAGGACCA |
| siRNA 2430 | 2430 | GGUCCUGUUCCAGAACCUG | 8784 | CAGGUUCUGGAACAGGACC |
| siRNA 2431 | 2431 | GUCCUGUUCCAGAACCUGA | 8785 | UCAGGUUCUGGAACAGGAC |
| siRNA 2432 | 2432 | UCCUGUUCCAGAACCUGAG | 8786 | CUCAGGUUCUGGAACAGGA |
| siRNA 2433 | 2433 | CCUGUUCCAGAACCUGAGU | 8787 | ACUCAGGUUCUGGAACAGG |
| siRNA 2434 | 2434 | CUGUUCCAGAACCUGAGUA | 8788 | UACUCAGGUUCUGGAACAG |
| siRNA 2435 | 2435 | UGUUCCAGAACCUGAGUAU | 8789 | AUACUCAGGUUCUGGAACA |
| siRNA 2436 | 2436 | GUUCCAGAACCUGAGUAUC | 8790 | GAUACUCAGGUUCUGGAAC |
| siRNA 2437 | 2437 | UUCCAGAACCUGAGUAUCU | 8791 | AGAUACUCAGGUUCUGGA |
| | | | | A |
| siRNA 2438 | 2438 | UCCAGAACCUGAGUAUCUG | 8792 | CAGAUACUCAGGUUCUGGA |
| siRNA 2439 | 2439 | CCAGAACCUGAGUAUCUGC | 8793 | GCAGAUACUCAGGUUCUGG |
| siRNA 2440 | 2440 | CAGAACCUGAGUAUCUGCA | 8794 | UGCAGAUACUCAGGUUCUG |
| siRNA 2441 | 2441 | AGAACCUGAGUAUCUGCAA | 8795 | UUGCAGAUACUCAGGUUCU |
| siRNA 2442 | 2442 | GAACCUGAGUAUCUGCAAA | 8796 | UUUGCAGAUACUCAGGUUC |
| siRNA 2443 | 2443 | AACCUGAGUAUCUGCAAAA | 8797 | UUUUGCAGAUACUCAGGU |
| | | | | U |
| siRNA 2444 | 2444 | ACCUGAGUAUCUGCAAAAG | 8798 | CUUUUGCAGAUACUCAGGU |
| siRNA 2445 | 2445 | CCUGAGUAUCUGCAAAAGU | 8799 | ACUUUUGCAGAUACUCAGG |
| siRNA 2446 | 2446 | CUGAGUAUCUGCAAAAGU | 8800 | AACUUUUGCAGAUACUCAG |
| | | U | | |
| siRNA 2447 | 2447 | UGAGUAUCUGCAAAAGUU | 8801 | CAACUUUUGCAGAUACUCA |
| | | G | | |
| siRNA 2448 | 2448 | GAGUAUCUGCAAAAGUUG | 8802 | GCAACUUUUGCAGAUACUC |
| | | C | | |
| siRNA 2449 | 2449 | AGUAUCUGCAAAAGUUGC | 8803 | UGCAACUUUUGCAGAUACU |
| | | A | | |
| siRNA 2450 | 2450 | GUAUCUGCAAAAGUUGCAC | 8804 | GUGCAACUUUUGCAGAUAC |
| siRNA 2451 | 2451 | UAUCUGCAAAAGUUGCACA | 8805 | UGUGCAACUUUUGCAGAU |
| | | | | A |
| siRNA 2452 | 2452 | AUCUGCAAAAGUUGCACAA | 8806 | UUGUGCAACUUUUGCAGA |
| | | | | U |
| siRNA 2453 | 2453 | UCUGCAAAAGUUGCACAAA | 8807 | UUUGUGCAACUUUUGCAG |
| | | | | A |
| siRNA 2454 | 2454 | CUGCAAAAGUUGCACAAAU | 8808 | AUUUGUGCAACUUUUGCA |
| | | | | G |
| siRNA 2455 | 2455 | UGCAAAAGUUGCACAAAU | 8809 | UAUUUGUGCAACUUUUGC |
| | | A | | A |
| siRNA 2456 | 2456 | GCAAAAGUUGCACAAAUAC | 8810 | GUAUUUGUGCAACUUUUG |
| | | | | C |
| siRNA 2457 | 2457 | CAAAAGUUGCACAAAUACC | 8811 | GGUAUUUGUGCAACUUUU |
| | | | | G |
| siRNA 2458 | 2458 | AAAAGUUGCACAAAUACCU | 8812 | AGGUAUUUGUGCAACUUU |
| | | | | U |
| siRNA 2459 | 2459 | AAAGUUGCACAAAUACCUA | 8813 | UAGGUAUUUGUGCAACUU |
| | | | | U |
| siRNA 2460 | 2460 | AAGUUGCACAAAUACCUAA | 8814 | UUAGGUAUUUGUGCAACU |
| | | | | U |
| siRNA 2461 | 2461 | AGUUGCACAAAUACCUAAU | 8815 | AUUAGGUAUUUGUGCAAC |
| | | | | U |
| siRNA 2462 | 2462 | GUUGCACAAAUACCUAAUA | 8816 | UAUUAGGUAUUUGUGCAA |
| | | | | C |
| siRNA 2463 | 2463 | UUGCACAAAUACCUAAUAA | 8817 | UUAUUAGGUAUUUGUGCA |
| | | | | A |
| siRNA 2464 | 2464 | UGCACAAAUACCUAAUAAC | 8818 | GUUAUUAGGUAUUUGUGC |
| | | | | A |
| siRNA 2465 | 2465 | GCACAAAUACCUAAUAACC | 8819 | GGUUAUUAGGUAUUUGUG |
| | | | | C |
| siRNA 2466 | 2466 | CACAAAUACCUAAUAACCA | 8820 | UGGUUAUUAGGUAUUUGU |
| | | | | G |
| siRNA 2467 | 2467 | ACAAAUACCUAAUAACCAG | 8821 | CUGGUUAUUAGGUAUUUG |
| | | | | U |
| siRNA 2468 | 2468 | CAAAUACCUAAUAACCAGA | 8822 | UCUGGUUAUUAGGUAUUU |
| | | | | G |
| siRNA 2469 | 2469 | AAAUACCUAAUAACCAGAA | 8823 | UUCUGGUUAUUAGGUAUU |
| | | | | U |
| siRNA 2470 | 2470 | AAUACCUAAUAACCAGAAC | 8824 | GUUCUGGUUAUUAGGUAU |
| | | | | U |
| siRNA 2471 | 2471 | AUACCUAAUAACCAGAACA | 8825 | UGUUCUGGUUAUUAGGUA |
| | | | | U |
| siRNA 2472 | 2472 | UACCUAAUAACCAGAACAG | 8826 | CUGUUCUGGUUAUUAGGU |
| | | | | A |
| siRNA 2473 | 2473 | ACCUAAUAACCAGAACAGA | 8827 | UCUGUUCUGGUUAUUAGG |
| | | | | U |
| siRNA 2474 | 2474 | CCUAAUAACCAGAACAGAA | 8828 | UUCUGUUCUGGUUAUUAG |
| | | | | G |
| siRNA 2475 | 2475 | CUAAUAACCAGAACAGAAA | 8829 | UUUCUGUUCUGGUUAUUA |
| | | | | G |
| siRNA 2476 | 2476 | UAAUAACCAGAACAGAAA | 8830 | CUUUCUGUUCUGGUUAUU |
| | | G | | A |
| siRNA 2477 | 2477 | AAUAACCAGAACAGAAAG | 8831 | UCUUUCUGUUCUGGUUAU |
| | | A | | U |
| siRNA 2478 | 2478 | AUAACCAGAACAGAAAGA | 8832 | UUCUUUCUGUUCUGGUUA |
| | | A | | U |
| siRNA 2479 | 2479 | UAACCAGAACAGAAAGAA | 8833 | UUUCUUUCUGUUCUGGUU |
| | | A | | A |
| siRNA 2480 | 2480 | AACCAGAACAGAAAGAAA | 8834 | AUUUCUUUCUGUUCUGGU |
| | | U | | U |
| siRNA 2481 | 2481 | ACCAGAACAGAAAGAAAU | 8835 | CAUUUCUUUCUGUUCUGGU |
| | | G | | |
| siRNA 2482 | 2482 | CCAGAACAGAAAGAAAUG | 8836 | ACAUUUCUUUCUGUUCUGG |
| | | U | | |
| siRNA 2483 | 2483 | CAGAACAGAAAGAAAUGU | 8837 | AACAUUUCUUUCUGUUCUG |
| | | U | | |
| siRNA 2484 | 2484 | AGAACAGAAAGAAAUGUU | 8838 | CAACAUUUCUUUCUGUUCU |
| | | G | | |
| siRNA 2485 | 2485 | GAACAGAAAGAAAUGUUG | 8839 | GCAACAUUUCUUUCUGUUC |
| | | C | | |
| siRNA 2486 | 2486 | AACAGAAAGAAAUGUUGC | 8840 | UGCAACAUUUCUUUCUGUU |
| | | A | | |
| siRNA 2487 | 2487 | ACAGAAAGAAAUGUUGCA | 8841 | CUGCAACAUUUCUUUCUGU |
| | | G | | |
| siRNA 2488 | 2488 | CAGAAAGAAAUGUUGCAG | 8842 | ACUGCAACAUUUCUUUCUG |
| | | U | | |
| siRNA 2489 | 2489 | AGAAAGAAAUGUUGCAGU | 8843 | UACUGCAACAUUUCUUUCU |
| | | A | | |
| siRNA 2490 | 2490 | GAAAGAAAUGUUGCAGUA | 8844 | AUACUGCAACAUUUCUUUC |
| | | U | | |
| siRNA 2491 | 2491 | AAAGAAAUGUUGCAGUAU | 8845 | UAUACUGCAACAUUUCUUU |
| | | A | | |
| siRNA 2492 | 2492 | AAGAAAUGUUGCAGUAUA | 8846 | AUAUACUGCAACAUUUCUU |
| | | U | | |
| siRNA 2493 | 2493 | AGAAAUGUUGCAGUAUAU | 8847 | CAUAUACUGCAACAUUUCU |
| | | G | | |
| siRNA 2494 | 2494 | GAAAUGUUGCAGUAUAUG | 8848 | GCAUAUACUGCAACAUUUC |
| | | C | | |
| siRNA 2495 | 2495 | AAAUGUUGCAGUAUAUGC | 8849 | AGCAUAUACUGCAACAUUU |
| | | U | | |
| siRNA 2496 | 2496 | AAUGUUGCAGUAUAUGCU | 8850 | CAGCAUAUACUGCAACAUU |
| | | G | | |
| siRNA 2497 | 2497 | AUGUUGCAGUAUAUGCUG | 8851 | UCAGCAUAUACUGCAACAU |
| | | A | | |
| siRNA 2498 | 2498 | UGUUGCAGUAUAUGCUGA | 8852 | CUCAGCAUAUACUGCAACA |
| | | G | | |
| siRNA 2499 | 2499 | GUUGCAGUAUAUGCUGAG | 8853 | UCUCAGCAUAUACUGCAAC |
| | | A | | |
| siRNA 2500 | 2500 | UUGCAGUAUAUGCUGAGA | 8854 | CUCUCAGCAUAUACUGCAA |
| | | G | | |
| siRNA 2501 | 2501 | UGCAGUAUAUGCUGAGAG | 8855 | ACUCUCAGCAUAUACUGCA |
| | | U | | |
| siRNA 2502 | 2502 | GCAGUAUAUGCUGAGAGU | 8856 | CACUCUCAGCAUAUACUGC |
| | | G | | |
| siRNA 2503 | 2503 | CAGUAUAUGCUGAGAGUG | 8857 | GCACUCUCAGCAUAUACUG |
| | | C | | |
| siRNA 2504 | 2504 | AGUAUAUGCUGAGAGUGC | 8858 | GGCACUCUCAGCAUAUACU |
| | | C | | |
| siRNA 2505 | 2505 | GUAUAUGCUGAGAGUGCC | 8859 | UGGCACUCUCAGCAUAUAC |
| | | A | | |
| siRNA 2506 | 2506 | UAUAUGCUGAGAGUGCCAC | 8860 | GUGGCACUCUCAGCAUAUA |
| siRNA 2507 | 2507 | AUAUGCUGAGAGUGCCACA | 8861 | UGUGGCACUCUCAGCAUAU |
| siRNA 2508 | 2508 | UAUGCUGAGAGUGCCACAU | 8862 | AUGUGGCACUCUCAGCAUA |
| siRNA 2509 | 2509 | AUGCUGAGAGUGCCACAUA | 8863 | UAUGUGGCACUCUCAGCAU |
| siRNA 2510 | 2510 | UGCUGAGAGUGCCACAUAU | 8864 | AUAUGUGGCACUCUCAGCA |
| siRNA 2511 | 2511 | GCUGAGAGUGCCACAUAUU | 8865 | AAUAUGUGGCACUCUCAGC |
| siRNA 2512 | 2512 | CUGAGAGUGCCACAUAUUG | 8866 | CAAUAUGUGGCACUCUCAG |
| siRNA 2513 | 2513 | UGAGAGUGCCACAUAUUG | 8867 | ACAAUAUGUGGCACUCUCA |
| | | U | | |
| siRNA 2514 | 2514 | GAGAGUGCCACAUAUUGUC | 8868 | GACAAUAUGUGGCACUCUC |
| siRNA 2515 | 2515 | AGAGUGCCACAUAUUGUCU | 8869 | AGACAAUAUGUGGCACUCU |
| siRNA 2516 | 2516 | GAGUGCCACAUAUUGUCUU | 8870 | AAGACAAUAUGUGGCACUC |
| siRNA 2517 | 2517 | AGUGCCACAUAUUGUCUUG | 8871 | CAAGACAAUAUGUGGCACU |
| siRNA 2518 | 2518 | GUGCCACAUAUUGUCUUGU | 8872 | ACAAGACAAUAUGUGGCAC |
| siRNA 2519 | 2519 | UGCCACAUAUUGUCUUGUG | 8873 | CACAAGACAAUAUGUGGCA |
| siRNA 2520 | 2520 | GCCACAUAUUGUCUUGUGA | 8874 | UCACAAGACAAUAUGUGGC |
| siRNA 2521 | 2521 | CCACAUAUUGUCUUGUGAA | 8875 | UUCACAAGACAAUAUGUG |
| | | | | G |
| siRNA 2522 | 2522 | CACAUAUUGUCUUGUGAA | 8876 | CUUCACAAGACAAUAUGUG |
| | | G | | |
| siRNA 2523 | 2523 | ACAUAUUGUCUUGUGAAG | 8877 | UCUUCACAAGACAAUAUGU |
| | | A | | |
| siRNA 2524 | 2524 | CAUAUUGUCUUGUGAAGA | 8878 | UUCUUCACAAGACAAUAUG |
| | | A | | |
| siRNA 2525 | 2525 | AUAUUGUCUUGUGAAGAA | 8879 | AUUCUUCACAAGACAAUAU |
| | | U | | |
| siRNA 2526 | 2526 | UAUUGUCUUGUGAAGAAU | 8880 | CAUUCUUCACAAGACAAUA |
| | | G | | |
| siRNA 2527 | 2527 | AUUGUCUUGUGAAGAAUG | 8881 | GCAUUCUUCACAAGACAAU |
| | | C | | |
| siRNA 2528 | 2528 | UUGUCUUGUGAAGAAUGC | 8882 | AGCAUUCUUCACAAGACAA |
| | | U | | |
| siRNA 2529 | 2529 | UGUCUUGUGAAGAAUGCU | 8883 | CAGCAUUCUUCACAAGACA |
| | | G | | |
| siRNA 2530 | 2530 | GUCUUGUGAAGAAUGCUG | 8884 | ACAGCAUUCUUCACAAGAC |
| | | U | | |
| siRNA 2531 | 2531 | UCUUGUGAAGAAUGCUGU | 8885 | CACAGCAUUCUUCACAAGA |
| | | G | | |
| siRNA 2532 | 2532 | CUUGUGAAGAAUGCUGUG | 8886 | UCACAGCAUUCUUCACAAG |
| | | A | | |
| siRNA 2533 | 2533 | UUGUGAAGAAUGCUGUGA | 8887 | UUCACAGCAUUCUUCACAA |
| | | A | | |
| siRNA 2534 | 2534 | UGUGAAGAAUGCUGUGAA | 8888 | UUUCACAGCAUUCUUCACA |
| | | A | | |
| siRNA 2535 | 2535 | GUGAAGAAUGCUGUGAAA | 8889 | UUUUCACAGCAUUCUUCAC |
| | | A | | |
| siRNA 2536 | 2536 | UGAAGAAUGCUGUGAAAA | 8890 | AUUUUCACAGCAUUCUUCA |
| | | U | | |
| siRNA 2537 | 2537 | GAAGAAUGCUGUGAAAAU | 8891 | CAUUUUCACAGCAUUCUUC |
| | | G | | |
| siRNA 2538 | 2538 | AAGAAUGCUGUGAAAAUG | 8892 | ACAUUUUCACAGCAUUCUU |
| | | U | | |
| siRNA 2539 | 2539 | AGAAUGCUGUGAAAAUGU | 8893 | AACAUUUUCACAGCAUUCU |
| | | U | | |
| siRNA 2540 | 2540 | GAAUGCUGUGAAAAUGUU | 8894 | AAACAUUUUCACAGCAUUC |
| | | U | | |
| siRNA 2541 | 2541 | AAUGCUGUGAAAAUGUUU | 8895 | UAAACAUUUUCACAGCAUU |
| | | A | | |
| siRNA 2542 | 2542 | AUGCUGUGAAAAUGUUUA | 8896 | UUAAACAUUUUCACAGCAU |
| | | A | | |
| siRNA 2543 | 2543 | UGCUGUGAAAAUGUUUAA | 8897 | CUUAAACAUUUUCACAGCA |
| | | G | | |
| siRNA 2544 | 2544 | GCUGUGAAAAUGUUUAAG | 8898 | CCUUAAACAUUUUCACAGC |
| | | G | | |
| siRNA 2545 | 2545 | CUGUGAAAAUGUUUAAGG | 8899 | UCCUUAAACAUUUUCACAG |
| | | A | | |
| siRNA 2546 | 2546 | UGUGAAAAUGUUUAAGGA | 8900 | AUCCUUAAACAUUUUCACA |
| | | U | | |
| siRNA 2547 | 2547 | GUGAAAAUGUUUAAGGAU | 8901 | UAUCCUUAAACAUUUUCAC |
| | | A | | |
| siRNA 2548 | 2548 | UGAAAAUGUUUAAGGAUA | 8902 | AUAUCCUUAAACAUUUUCA |
| | | U | | |
| siRNA 2549 | 2549 | GAAAAUGUUUAAGGAUAU | 8903 | AAUAUCCUUAAACAUUUUC |
| | | U | | |
| siRNA 2550 | 2550 | AAAAUGUUUAAGGAUAUU | 8904 | CAAUAUCCUUAAACAUUUU |
| | | G | | |
| siRNA 2551 | 2551 | AAAUGUUUAAGGAUAUUG | 8905 | CCAAUAUCCUUAAACAUUU |
| | | G | | |
| siRNA 2552 | 2552 | AAUGUUUAAGGAUAUUGG | 8906 | CCCAAUAUCCUUAAACAUU |
| | | G | | |
| siRNA 2553 | 2553 | AUGUUUAAGGAUAUUGGG | 8907 | CCCCAAUAUCCUUAAACAU |
| | | G | | |
| siRNA 2554 | 2554 | UGUUUAAGGAUAUUGGGG | 8908 | ACCCCAAUAUCCUUAAACA |
| | | U | | |
| siRNA 2555 | 2555 | GUUUAAGGAUAUUGGGGU | 8909 | AACCCCAAUAUCCUUAAAC |
| | | U | | |
| siRNA 2556 | 2556 | UUUAAGGAUAUUGGGGUU | 8910 | AAACCCCAAUAUCCUUAAA |
| | | U | | |
| siRNA 2557 | 2557 | UUAAGGAUAUUGGGGUUU | 8911 | AAAACCCCAAUAUCCUUAA |
| | | U | | |
| siRNA 2558 | 2558 | UAAGGAUAUUGGGGUUUU | 8912 | GAAAACCCCAAUAUCCUUA |
| | | C | | |
| siRNA 2559 | 2559 | AAGGAUAUUGGGGUUUUC | 8913 | UGAAAACCCCAAUAUCCUU |
| | | A | | |
| siRNA 2560 | 2560 | AGGAUAUUGGGGUUUUCA | 8914 | UUGAAAACCCCAAUAUCCU |
| | | A | | |
| siRNA 2561 | 2561 | GGAUAUUGGGGUUUUCAA | 8915 | CUUGAAAACCCCAAUAUCC |
| | | G | | |
| siRNA 2562 | 2562 | GAUAUUGGGGUUUUCAAG | 8916 | CCUUGAAAACCCCAAUAUC |
| | | G | | |
| siRNA 2563 | 2563 | AUAUUGGGGUUUUCAAGG | 8917 | UCCUUGAAAACCCCAAUAU |
| | | A | | |
| siRNA 2564 | 2564 | UAUUGGGGUUUUCAAGGA | 8918 | CUCCUUGAAAACCCCAAUA |
| | | G | | |
| siRNA 2565 | 2565 | AUUGGGGUUUUCAAGGAG | 8919 | UCUCCUUGAAAACCCCAAU |
| | | A | | |
| siRNA 2566 | 2566 | UUGGGGUUUUCAAGGAGA | 8920 | GUCUCCUUGAAAACCCCAA |
| | | C | | |
| siRNA 2567 | 2567 | UGGGGUUUUCAAGGAGAC | 8921 | GGUCUCCUUGAAAACCCCA |
| | | C | | |
| siRNA 2568 | 2568 | GGGGUUUUCAAGGAGACC | 8922 | UGGUCUCCUUGAAAACCCC |
| | | A | | |
| siRNA 2569 | 2569 | GGGUUUUCAAGGAGACCA | 8923 | UUGGUCUCCUUGAAAACCC |
| | | A | | |
| siRNA 2570 | 2570 | GGUUUUCAAGGAGACCAA | 8924 | UUUGGUCUCCUUGAAAACC |
| | | A | | |
| siRNA 2571 | 2571 | GUUUUCAAGGAGACCAAAC | 8925 | GUUUGGUCUCCUUGAAAAC |
| siRNA 2572 | 2572 | UUUUCAAGGAGACCAAACA | 8926 | UGUUUGGUCUCCUUGAAA |
| | | | | A |
| siRNA 2573 | 2573 | UUUCAAGGAGACCAAACAA | 8927 | UUGUUUGGUCUCCUUGAA |
| | | | | A |
| siRNA 2574 | 2574 | UUCAAGGAGACCAAACAAA | 8928 | UUUGUUUGGUCUCCUUGA |
| | | | | A |
| siRNA 2575 | 2575 | UCAAGGAGACCAAACAAAA | 8929 | UUUUGUUUGGUCUCCUUG |
| | | | | A |
| siRNA 2576 | 2576 | CAAGGAGACCAAACAAAAG | 8930 | CUUUUGUUUGGUCUCCUUG |
| siRNA 2577 | 2577 | AAGGAGACCAAACAAAAG | 8931 | UCUUUUGUUUGGUCUCCUU |
| | | A | | |
| siRNA 2578 | 2578 | AGGAGACCAAACAAAAGA | 8932 | CUCUUUUGUUUGGUCUCCU |
| | | G | | |
| siRNA 2579 | 2579 | GGAGACCAAACAAAAGAG | 8933 | UCUCUUUUGUUUGGUCUCC |
| | | A | | |
| siRNA 2580 | 2580 | GAGACCAAACAAAAGAGA | 8934 | CUCUCUUUUGUUUGGUCUC |
| | | G | | |
| siRNA 2581 | 2581 | AGACCAAACAAAAGAGAG | 8935 | ACUCUCUUUUGUUUGGUCU |
| | | U | | |
| siRNA 2582 | 2582 | GACCAAACAAAAGAGAGU | 8936 | CACUCUCUUUUGUUUGGUC |
| | | G | | |
| siRNA 2583 | 2583 | ACCAAACAAAAGAGAGUG | 8937 | ACACUCUCUUUUGUUUGGU |
| | | U | | |
| siRNA 2584 | 2584 | CCAAACAAAAGAGAGUGUC | 8938 | GACACUCUCUUUUGUUUGG |
| siRNA 2585 | 2585 | CAAACAAAAGAGAGUGUC | 8939 | AGACACUCUCUUUUGUUUG |
| | | U | | |
| siRNA 2586 | 2586 | AAACAAAAGAGAGUGUCU | 8940 | CAGACACUCUCUUUUGUUU |
| | | G | | |
| siRNA 2587 | 2587 | AACAAAAGAGAGUGUCUG | 8941 | ACAGACACUCUCUUUUGUU |
| | | U | | |
| siRNA 2588 | 2588 | ACAAAAGAGAGUGUCUGU | 8942 | AACAGACACUCUCUUUUGU |
| | | U | | |
| siRNA 2589 | 2589 | CAAAAGAGAGUGUCUGUU | 8943 | AAACAGACACUCUCUUUUG |
| | | U | | |
| siRNA 2590 | 2590 | AAAAGAGAGUGUCUGUUU | 8944 | AAAACAGACACUCUCUUUU |
| | | U | | |
| siRNA 2591 | 2591 | AAAGAGAGUGUCUGUUUU | 8945 | UAAAACAGACACUCUCUUU |
| | | A | | |
| siRNA 2592 | 2592 | AAGAGAGUGUCUGUUUUA | 8946 | CUAAAACAGACACUCUCUU |
| | | G | | |
| siRNA 2593 | 2593 | AGAGAGUGUCUGUUUUAG | 8947 | UCUAAAACAGACACUCUCU |
| | | A | | |
| siRNA 2594 | 2594 | GAGAGUGUCUGUUUUAGA | 8948 | UUCUAAAACAGACACUCUC |
| | | A | | |
| siRNA 2595 | 2595 | AGAGUGUCUGUUUUAGAA | 8949 | GUUCUAAAACAGACACUCU |
| | | C | | |
| siRNA 2596 | 2596 | GAGUGUCUGUUUUAGAAC | 8950 | AGUUCUAAAACAGACACUC |
| | | U | | |
| siRNA 2597 | 2597 | AGUGUCUGUUUUAGAACU | 8951 | CAGUUCUAAAACAGACACU |
| | | G | | |
| siRNA 2598 | 2598 | GUGUCUGUUUUAGAACUG | 8952 | UCAGUUCUAAAACAGACAC |
| | | A | | |
| siRNA 2599 | 2599 | UGUCUGUUUUAGAACUGA | 8953 | CUCAGUUCUAAAACAGACA |
| | | G | | |
| siRNA 2600 | 2600 | GUCUGUUUUAGAACUGAG | 8954 | GCUCAGUUCUAAAACAGAC |
| | | C | | |
| siRNA 2601 | 2601 | UCUGUUUUAGAACUGAGC | 8955 | UGCUCAGUUCUAAAACAGA |
| | | A | | |
| siRNA 2602 | 2602 | CUGUUUUAGAACUGAGCA | 8956 | CUGCUCAGUUCUAAAACAG |
| | | G | | |
| siRNA 2603 | 2603 | UGUUUUAGAACUGAGCAG | 8957 | GCUGCUCAGUUCUAAAACA |
| | | C | | |
| siRNA 2604 | 2604 | GUUUUAGAACUGAGCAGC | 8958 | UGCUGCUCAGUUCUAAAAC |
| | | A | | |
| siRNA 2605 | 2605 | UUUUAGAACUGAGCAGCAC | 8959 | GUGCUGCUCAGUUCUAAAA |
| siRNA 2606 | 2606 | UUUAGAACUGAGCAGCACU | 8960 | AGUGCUGCUCAGUUCUAAA |
| siRNA 2607 | 2607 | UUAGAACUGAGCAGCACUU | 8961 | AAGUGCUGCUCAGUUCUAA |
| siRNA 2608 | 2608 | UAGAACUGAGCAGCACUUU | 8962 | AAAGUGCUGCUCAGUUCUA |
| siRNA 2609 | 2609 | AGAACUGAGCAGCACUUUU | 8963 | AAAAGUGCUGCUCAGUUCU |
| siRNA 2610 | 2610 | GAACUGAGCAGCACUUUUC | 8964 | GAAAAGUGCUGCUCAGUUC |
| siRNA 2611 | 2611 | AACUGAGCAGCACUUUUCU | 8965 | AGAAAAGUGCUGCUCAGU |
| | | | | U |
| siRNA 2612 | 2612 | ACUGAGCAGCACUUUUCUA | 8966 | UAGAAAAGUGCUGCUCAG |
| | | | | U |
| siRNA 2613 | 2613 | CUGAGCAGCACUUUUCUAC | 8967 | GUAGAAAAGUGCUGCUCA |
| | | | | G |
| siRNA 2614 | 2614 | UGAGCAGCACUUUUCUACC | 8968 | GGUAGAAAAGUGCUGCUC |
| | | | | A |
| siRNA 2615 | 2615 | GAGCAGCACUUUUCUACCU | 8969 | AGGUAGAAAAGUGCUGCU |
| | | | | C |
| siRNA 2616 | 2616 | AGCAGCACUUUUCUACCUC | 8970 | GAGGUAGAAAAGUGCUGC |
| | | | | U |
| siRNA 2617 | 2617 | GCAGCACUUUUCUACCUCA | 8971 | UGAGGUAGAAAAGUGCUG |
| | | | | C |
| siRNA 2618 | 2618 | CAGCACUUUUCUACCUCAA | 8972 | UUGAGGUAGAAAAGUGCU |
| | | | | G |
| siRNA 2619 | 2619 | AGCACUUUUCUACCUCAAU | 8973 | AUUGAGGUAGAAAAGUGC |
| | | | | U |
| siRNA 2620 | 2620 | GCACUUUUCUACCUCAAUG | 8974 | CAUUGAGGUAGAAAAGUG |
| | | | | C |
| siRNA 2621 | 2621 | CACUUUUCUACCUCAAUGC | 8975 | GCAUUGAGGUAGAAAAGU |
| | | | | G |
| siRNA 2622 | 2622 | ACUUUUCUACCUCAAUGCA | 8976 | UGCAUUGAGGUAGAAAAG |
| | | | | U |
| siRNA 2623 | 2623 | CUUUUCUACCUCAAUGCAA | 8977 | UUGCAUUGAGGUAGAAAA |
| | | | | G |
| siRNA 2624 | 2624 | UUUUCUACCUCAAUGCAAC | 8978 | GUUGCAUUGAGGUAGAAA |
| | | | | A |
| siRNA 2625 | 2625 | UUUCUACCUCAAUGCAACC | 8979 | GGUUGCAUUGAGGUAGAA |
| | | | | A |
| siRNA 2626 | 2626 | UUCUACCUCAAUGCAACCG | 8980 | CGGUUGCAUUGAGGUAGA |
| | | | | A |
| siRNA 2627 | 2627 | UCUACCUCAAUGCAACCGA | 8981 | UCGGUUGCAUUGAGGUAG |
| | | | | A |
| siRNA 2628 | 2628 | CUACCUCAAUGCAACCGAC | 8982 | GUCGGUUGCAUUGAGGUA |
| | | | | G |
| siRNA 2629 | 2629 | UACCUCAAUGCAACCGACA | 8983 | UGUCGGUUGCAUUGAGGU |
| | | | | A |
| siRNA 2630 | 2630 | ACCUCAAUGCAACCGACAA | 8984 | UUGUCGGUUGCAUUGAGG |
| | | | | U |
| siRNA 2631 | 2631 | CCUCAAUGCAACCGACAAA | 8985 | UUUGUCGGUUGCAUUGAG |
| | | | | G |
| siRNA 2632 | 2632 | CUCAAUGCAACCGACAAAA | 8986 | UUUUGUCGGUUGCAUUGA |
| | | | | G |
| siRNA 2633 | 2633 | UCAAUGCAACCGACAAAAA | 8987 | UUUUUGUCGGUUGCAUUG |
| | | | | A |
| siRNA 2634 | 2634 | CAAUGCAACCGACAAAAAC | 8988 | GUUUUUGUCGGUUGCAUU |
| | | | | G |
| siRNA 2635 | 2635 | AAUGCAACCGACAAAAACU | 8989 | AGUUUUUGUCGGUUGCAU |
| | | | | U |
| siRNA 2636 | 2636 | AUGCAACCGACAAAAACUU | 8990 | AAGUUUUUGUCGGUUGCA |
| | | | | U |
| siRNA 2637 | 2637 | UGCAACCGACAAAAACUUC | 8991 | GAAGUUUUUGUCGGUUGC |
| | | | | A |
| siRNA 2638 | 2638 | GCAACCGACAAAAACUUCU | 8992 | AGAAGUUUUUGUCGGUUG |
| | | | | C |
| siRNA 2639 | 2639 | CAACCGACAAAAACUUCUA | 8993 | UAGAAGUUUUUGUCGGUU |
| | | | | G |
| siRNA 2640 | 2640 | AACCGACAAAAACUUCUAG | 8994 | CUAGAAGUUUUUGUCGGU |
| | | | | U |
| siRNA 2641 | 2641 | ACCGACAAAAACUUCUAGA | 8995 | UCUAGAAGUUUUUGUCGG |
| | | | | U |
| siRNA 2642 | 2642 | CCGACAAAAACUUCUAGAA | 8996 | UUCUAGAAGUUUUUGUCG |
| | | | | G |
| siRNA 2643 | 2643 | CGACAAAAACUUCUAGAAU | 8997 | AUUCUAGAAGUUUUUGUC |
| | | | | G |
| siRNA 2644 | 2644 | GACAAAAACUUCUAGAAU | 8998 | UAUUCUAGAAGUUUUUGU |
| | | A | | C |
| siRNA 2645 | 2645 | ACAAAAACUUCUAGAAUA | 8999 | AUAUUCUAGAAGUUUUUG |
| | | U | | U |
| siRNA 2646 | 2646 | CAAAAACUUCUAGAAUAU | 9000 | UAUAUUCUAGAAGUUUUU |
| | | A | | G |
| siRNA 2647 | 2647 | AAAAACUUCUAGAAUAUA | 9001 | AUAUAUUCUAGAAGUUUU |
| | | U | | U |
| siRNA 2648 | 2648 | AAAACUUCUAGAAUAUAU | 9002 | AAUAUAUUCUAGAAGUUU |
| | | U | | U |
| siRNA 2649 | 2649 | AAACUUCUAGAAUAUAUU | 9003 | GAAUAUAUUCUAGAAGUU |
| | | C | | U |
| siRNA 2650 | 2650 | AACUUCUAGAAUAUAUUC | 9004 | AGAAUAUAUUCUAGAAGU |
| | | U | | U |
| siRNA 2651 | 2651 | ACUUCUAGAAUAUAUUCU | 9005 | CAGAAUAUAUUCUAGAAG |
| | | G | | U |
| siRNA 2652 | 2652 | CUUCUAGAAUAUAUUCUG | 9006 | UCAGAAUAUAUUCUAGAA |
| | | A | | G |
| siRNA 2653 | 2653 | UUCUAGAAUAUAUUCUGA | 9007 | CUCAGAAUAUAUUCUAGA |
| | | G | | A |
| siRNA 2654 | 2654 | UCUAGAAUAUAUUCUGAG | 9008 | ACUCAGAAUAUAUUCUAG |
| | | U | | A |
| siRNA 2655 | 2655 | CUAGAAUAUAUUCUGAGU | 9009 | AACUCAGAAUAUAUUCUA |
| | | U | | G |
| siRNA 2656 | 2656 | UAGAAUAUAUUCUGAGUU | 9010 | AAACUCAGAAUAUAUUCU |
| | | U | | A |
| siRNA 2657 | 2657 | AGAAUAUAUUCUGAGUUU | 9011 | AAAACUCAGAAUAUAUUC |
| | | U | | U |
| siRNA 2658 | 2658 | GAAUAUAUUCUGAGUUUU | 9012 | CAAAACUCAGAAUAUAUUC |
| | | G | | |
| siRNA 2659 | 2659 | AAUAUAUUCUGAGUUUUG | 9013 | ACAAAACUCAGAAUAUAU |
| | | U | | U |
| siRNA 2660 | 2660 | AUAUAUUCUGAGUUUUGU | 9014 | CACAAAACUCAGAAUAUAU |
| | | G | | |
| siRNA 2661 | 2661 | UAUAUUCUGAGUUUUGUG | 9015 | CCACAAAACUCAGAAUAUA |
| | | G | | |
| siRNA 2662 | 2662 | AUAUUCUGAGUUUUGUGG | 9016 | ACCACAAAACUCAGAAUAU |
| | | U | | |
| siRNA 2663 | 2663 | UAUUCUGAGUUUUGUGGU | 9017 | CACCACAAAACUCAGAAUA |
| | | G | | |
| siRNA 2664 | 2664 | AUUCUGAGUUUUGUGGUG | 9018 | GCACCACAAAACUCAGAAU |
| | | C | | |
| siRNA 2665 | 2665 | UUCUGAGUUUUGUGGUGC | 9019 | AGCACCACAAAACUCAGAA |
| | | U | | |
| siRNA 2666 | 2666 | UCUGAGUUUUGUGGUGCU | 9020 | CAGCACCACAAAACUCAGA |
| | | G | | |
| siRNA 2667 | 2667 | CUGAGUUUUGUGGUGCUG | 9021 | ACAGCACCACAAAACUCAG |
| | | U | | |
| siRNA 2668 | 2668 | UGAGUUUUGUGGUGCUGU | 9022 | UACAGCACCACAAAACUCA |
| | | A | | |
| siRNA 2669 | 2669 | GAGUUUUGUGGUGCUGUA | 9023 | CUACAGCACCACAAAACUC |
| | | G | | |
| siRNA 2670 | 2670 | AGUUUUGUGGUGCUGUAG | 9024 | CCUACAGCACCACAAAACU |
| | | G | | |
| siRNA 2671 | 2671 | GUUUUGUGGUGCUGUAGG | 9025 | ACCUACAGCACCACAAAAC |
| | | U | | |
| siRNA 2672 | 2672 | UUUUGUGGUGCUGUAGGU | 9026 | UACCUACAGCACCACAAAA |
| | | A | | |
| siRNA 2673 | 2673 | UUUGUGGUGCUGUAGGUA | 9027 | UUACCUACAGCACCACAAA |
| | | A | | |
| siRNA 2674 | 2674 | UUGUGGUGCUGUAGGUAA | 9028 | GUUACCUACAGCACCACAA |
| | | C | | |
| siRNA 2675 | 2675 | UGUGGUGCUGUAGGUAAC | 9029 | CGUUACCUACAGCACCACA |
| | | G | | |
| siRNA 2676 | 2676 | GUGGUGCUGUAGGUAACG | 9030 | ACGUUACCUACAGCACCAC |
| | | U | | |
| siRNA 2677 | 2677 | UGGUGCUGUAGGUAACGU | 9031 | CACGUUACCUACAGCACCA |
| | | G | | |
| siRNA 2678 | 2678 | GGUGCUGUAGGUAACGUG | 9032 | ACACGUUACCUACAGCACC |
| | | U | | |
| siRNA 2679 | 2679 | GUGCUGUAGGUAACGUGU | 9033 | CACACGUUACCUACAGCAC |
| | | G | | |
| siRNA 2680 | 2680 | UGCUGUAGGUAACGUGUG | 9034 | CCACACGUUACCUACAGCA |
| | | G | | |
| siRNA 2681 | 2681 | GCUGUAGGUAACGUGUGG | 9035 | GCCACACGUUACCUACAGC |
| | | C | | |
| siRNA 2682 | 2682 | CUGUAGGUAACGUGUGGC | 9036 | UGCCACACGUUACCUACAG |
| | | A | | |
| siRNA 2683 | 2683 | UGUAGGUAACGUGUGGCA | 9037 | GUGCCACACGUUACCUACA |
| | | C | | |
| siRNA 2684 | 2684 | GUAGGUAACGUGUGGCAC | 9038 | AGUGCCACACGUUACCUAC |
| | | U | | |
| siRNA 2685 | 2685 | UAGGUAACGUGUGGCACU | 9039 | CAGUGCCACACGUUACCUA |
| | | G | | |
| siRNA 2686 | 2686 | AGGUAACGUGUGGCACUGC | 9040 | GCAGUGCCACACGUUACCU |
| siRNA 2687 | 2687 | GGUAACGUGUGGCACUGCU | 9041 | AGCAGUGCCACACGUUACC |
| siRNA 2688 | 2688 | GUAACGUGUGGCACUGCUG | 9042 | CAGCAGUGCCACACGUUAC |
| siRNA 2689 | 2689 | UAACGUGUGGCACUGCUGG | 9043 | CCAGCAGUGCCACACGUUA |
| siRNA 2690 | 2690 | AACGUGUGGCACUGCUGGC | 9044 | GCCAGCAGUGCCACACGUU |
| siRNA 2691 | 2691 | ACGUGUGGCACUGCUGGCA | 9045 | UGCCAGCAGUGCCACACGU |
| siRNA 2692 | 2692 | CGUGUGGCACUGCUGGCAA | 9046 | UUGCCAGCAGUGCCACACG |
| siRNA 2693 | 2693 | GUGUGGCACUGCUGGCAAA | 9047 | UUUGCCAGCAGUGCCACAC |
| siRNA 2694 | 2694 | UGUGGCACUGCUGGCAAAU | 9048 | AUUUGCCAGCAGUGCCACA |
| siRNA 2695 | 2695 | GUGGCACUGCUGGCAAAUG | 9049 | CAUUUGCCAGCAGUGCCAC |
| siRNA 2696 | 2696 | UGGCACUGCUGGCAAAUGA | 9050 | UCAUUUGCCAGCAGUGCCA |
| siRNA 2697 | 2697 | GGCACUGCUGGCAAAUGAA | 9051 | UUCAUUUGCCAGCAGUGCC |
| siRNA 2698 | 2698 | GCACUGCUGGCAAAUGAAG | 9052 | CUUCAUUUGCCAGCAGUGC |
| siRNA 2699 | 2699 | CACUGCUGGCAAAUGAAGG | 9053 | CCUUCAUUUGCCAGCAGUG |
| siRNA 2700 | 2700 | ACUGCUGGCAAAUGAAGG | 9054 | ACCUUCAUUUGCCAGCAGU |
| | | U | | |
| siRNA 2701 | 2701 | CUGCUGGCAAAUGAAGGUC | 9055 | GACCUUCAUUUGCCAGCAG |
| siRNA 2702 | 2702 | UGCUGGCAAAUGAAGGUC | 9056 | UGACCUUCAUUUGCCAGCA |
| | | A | | |
| siRNA 2703 | 2703 | GCUGGCAAAUGAAGGUCA | 9057 | AUGACCUUCAUUUGCCAGC |
| | | U | | |
| siRNA 2704 | 2704 | CUGGCAAAUGAAGGUCAU | 9058 | CAUGACCUUCAUUUGCCAG |
| | | G | | |
| siRNA 2705 | 2705 | UGGCAAAUGAAGGUCAUG | 9059 | UCAUGACCUUCAUUUGCCA |
| | | A | | |
| siRNA 2706 | 2706 | GGCAAAUGAAGGUCAUGA | 9060 | CUCAUGACCUUCAUUUGCC |
| | | G | | |
| siRNA 2707 | 2707 | GCAAAUGAAGGUCAUGAG | 9061 | UCUCAUGACCUUCAUUUGC |
| | | A | | |
| siRNA 2708 | 2708 | CAAAUGAAGGUCAUGAGA | 9062 | AUCUCAUGACCUUCAUUUG |
| | | U | | |
| siRNA 2709 | 2709 | AAAUGAAGGUCAUGAGAU | 9063 | CAUCUCAUGACCUUCAUUU |
| | | G | | |
| siRNA 2710 | 2710 | AAUGAAGGUCAUGAGAUG | 9064 | UCAUCUCAUGACCUUCAUU |
| | | A | | |
| siRNA 2711 | 2711 | AUGAAGGUCAUGAGAUGA | 9065 | CUCAUCUCAUGACCUUCAU |
| | | G | | |
| siRNA 2712 | 2712 | UGAAGGUCAUGAGAUGAG | 9066 | ACUCAUCUCAUGACCUUCA |
| | | U | | |
| siRNA 2713 | 2713 | GAAGGUCAUGAGAUGAGU | 9067 | AACUCAUCUCAUGACCUUC |
| | | U | | |
| siRNA 2714 | 2714 | AAGGUCAUGAGAUGAGUU | 9068 | GAACUCAUCUCAUGACCUU |
| | | C | | |
| siRNA 2715 | 2715 | AGGUCAUGAGAUGAGUUC | 9069 | GGAACUCAUCUCAUGACCU |
| | | C | | |
| siRNA 2716 | 2716 | GGUCAUGAGAUGAGUUCC | 9070 | AGGAACUCAUCUCAUGACC |
| | | U | | |
| siRNA 2717 | 2717 | GUCAUGAGAUGAGUUCCU | 9071 | AAGGAACUCAUCUCAUGAC |
| | | U | | |
| siRNA 2718 | 2718 | UCAUGAGAUGAGUUCCUU | 9072 | CAAGGAACUCAUCUCAUGA |
| | | G | | |
| siRNA 2719 | 2719 | CAUGAGAUGAGUUCCUUG | 9073 | ACAAGGAACUCAUCUCAUG |
| | | U | | |
| siRNA 2720 | 2720 | AUGAGAUGAGUUCCUUGU | 9074 | UACAAGGAACUCAUCUCAU |
| | | A | | |
| siRNA 2721 | 2721 | UGAGAUGAGUUCCUUGUA | 9075 | CUACAAGGAACUCAUCUCA |
| | | G | | |
| siRNA 2722 | 2722 | GAGAUGAGUUCCUUGUAG | 9076 | CCUACAAGGAACUCAUCUC |
| | | G | | |
| siRNA 2723 | 2723 | AGAUGAGUUCCUUGUAGG | 9077 | ACCUACAAGGAACUCAUCU |
| | | U | | |
| siRNA 2724 | 2724 | GAUGAGUUCCUUGUAGGU | 9078 | UACCUACAAGGAACUCAUC |
| | | A | | |
| siRNA 2725 | 2725 | AUGAGUUCCUUGUAGGUA | 9079 | GUACCUACAAGGAACUCAU |
| | | C | | |
| siRNA 2726 | 2726 | UGAGUUCCUUGUAGGUACC | 9080 | GGUACCUACAAGGAACUCA |
| siRNA 2727 | 2727 | GAGUUCCUUGUAGGUACCA | 9081 | UGGUACCUACAAGGAACUC |
| siRNA 2728 | 2728 | AGUUCCUUGUAGGUACCAG | 9082 | CUGGUACCUACAAGGAACU |
| siRNA 2729 | 2729 | GUUCCUUGUAGGUACCAGC | 9083 | GCUGGUACCUACAAGGAAC |
| siRNA 2730 | 2730 | UUCCUUGUAGGUACCAGCU | 9084 | AGCUGGUACCUACAAGGAA |
| siRNA 2731 | 2731 | UCCUUGUAGGUACCAGCUU | 9085 | AAGCUGGUACCUACAAGGA |
| siRNA 2732 | 2732 | CCUUGUAGGUACCAGCUUC | 9086 | GAAGCUGGUACCUACAAGG |
| siRNA 2733 | 2733 | CUUGUAGGUACCAGCUUCU | 9087 | AGAAGCUGGUACCUACAAG |
| siRNA 2734 | 2734 | UUGUAGGUACCAGCUUCUG | 9088 | CAGAAGCUGGUACCUACAA |
| siRNA 2735 | 2735 | UGUAGGUACCAGCUUCUGG | 9089 | CCAGAAGCUGGUACCUACA |
| siRNA 2736 | 2736 | GUAGGUACCAGCUUCUGGC | 9090 | GCCAGAAGCUGGUACCUAC |
| siRNA 2737 | 2737 | UAGGUACCAGCUUCUGGCU | 9091 | AGCCAGAAGCUGGUACCUA |
| siRNA 2738 | 2738 | AGGUACCAGCUUCUGGCUC | 9092 | GAGCCAGAAGCUGGUACCU |
| siRNA 2739 | 2739 | GGUACCAGCUUCUGGCUCA | 9093 | UGAGCCAGAAGCUGGUACC |
| siRNA 2740 | 2740 | GUACCAGCUUCUGGCUCAA | 9094 | UUGAGCCAGAAGCUGGUAC |
| siRNA 2741 | 2741 | UACCAGCUUCUGGCUCAAG | 9095 | CUUGAGCCAGAAGCUGGUA |
| siRNA 2742 | 2742 | ACCAGCUUCUGGCUCAAGA | 9096 | UCUUGAGCCAGAAGCUGGU |
| siRNA 2743 | 2743 | CCAGCUUCUGGCUCAAGAG | 9097 | CUCUUGAGCCAGAAGCUGG |
| siRNA 2744 | 2744 | CAGCUUCUGGCUCAAGAGU | 9098 | ACUCUUGAGCCAGAAGCUG |
| siRNA 2745 | 2745 | AGCUUCUGGCUCAAGAGUU | 9099 | AACUCUUGAGCCAGAAGCU |
| siRNA 2746 | 2746 | GCUUCUGGCUCAAGAGUUG | 9100 | CAACUCUUGAGCCAGAAGC |
| siRNA 2747 | 2747 | CUUCUGGCUCAAGAGUUGA | 9101 | UCAACUCUUGAGCCAGAAG |
| siRNA 2748 | 2748 | UUCUGGCUCAAGAGUUGA | 9102 | UUCAACUCUUGAGCCAGAA |
| | | A | | |
| siRNA 2749 | 2749 | UCUGGCUCAAGAGUUGAA | 9103 | CUUCAACUCUUGAGCCAGA |
| | | G | | |
| siRNA 2750 | 2750 | CUGGCUCAAGAGUUGAAG | 9104 | CCUUCAACUCUUGAGCCAG |
| | | G | | |
| siRNA 2751 | 2751 | UGGCUCAAGAGUUGAAGG | 9105 | ACCUUCAACUCUUGAGCCA |
| | | U | | |
| siRNA 2752 | 2752 | GGCUCAAGAGUUGAAGGU | 9106 | CACCUUCAACUCUUGAGCC |
| | | G | | |
| siRNA 2753 | 2753 | GCUCAAGAGUUGAAGGUG | 9107 | GCACCUUCAACUCUUGAGC |
| | | C | | |
| siRNA 2754 | 2754 | CUCAAGAGUUGAAGGUGCC | 9108 | GGCACCUUCAACUCUUGAG |
| siRNA 2755 | 2755 | UCAAGAGUUGAAGGUGCC | 9109 | UGGCACCUUCAACUCUUGA |
| | | A | | |
| siRNA 2756 | 2756 | CAAGAGUUGAAGGUGCCA | 9110 | AUGGCACCUUCAACUCUUG |
| | | U | | |
| siRNA 2757 | 2757 | AAGAGUUGAAGGUGCCAU | 9111 | GAUGGCACCUUCAACUCUU |
| | | C | | |
| siRNA 2758 | 2758 | AGAGUUGAAGGUGCCAUC | 9112 | CGAUGGCACCUUCAACUCU |
| | | G | | |
| siRNA 2759 | 2759 | GAGUUGAAGGUGCCAUCGC | 9113 | GCGAUGGCACCUUCAACUC |
| siRNA 2760 | 2760 | AGUUGAAGGUGCCAUCGCA | 9114 | UGCGAUGGCACCUUCAACU |
| siRNA 2761 | 2761 | GUUGAAGGUGCCAUCGCAG | 9115 | CUGCGAUGGCACCUUCAAC |
| siRNA 2762 | 2762 | UUGAAGGUGCCAUCGCAGG | 9116 | CCUGCGAUGGCACCUUCAA |
| siRNA 2763 | 2763 | UGAAGGUGCCAUCGCAGGG | 9117 | CCCUGCGAUGGCACCUUCA |
| siRNA 2764 | 2764 | GAAGGUGCCAUCGCAGGGU | 9118 | ACCCUGCGAUGGCACCUUC |
| siRNA 2765 | 2765 | AAGGUGCCAUCGCAGGGUC | 9119 | GACCCUGCGAUGGCACCUU |
| siRNA 2766 | 2766 | AGGUGCCAUCGCAGGGUCA | 9120 | UGACCCUGCGAUGGCACCU |
| siRNA 2767 | 2767 | GGUGCCAUCGCAGGGUCAG | 9121 | CUGACCCUGCGAUGGCACC |
| siRNA 2768 | 2768 | GUGCCAUCGCAGGGUCAGG | 9122 | CCUGACCCUGCGAUGGCAC |
| siRNA 2769 | 2769 | UGCCAUCGCAGGGUCAGGC | 9123 | GCCUGACCCUGCGAUGGCA |
| siRNA 2770 | 2770 | GCCAUCGCAGGGUCAGGCC | 9124 | GGCCUGACCCUGCGAUGGC |
| siRNA 2771 | 2771 | CCAUCGCAGGGUCAGGCCU | 9125 | AGGCCUGACCCUGCGAUGG |
| siRNA 2772 | 2772 | CAUCGCAGGGUCAGGCCUG | 9126 | CAGGCCUGACCCUGCGAUG |
| siRNA 2773 | 2773 | AUCGCAGGGUCAGGCCUGC | 9127 | GCAGGCCUGACCCUGCGAU |
| siRNA 2774 | 2774 | UCGCAGGGUCAGGCCUGCC | 9128 | GGCAGGCCUGACCCUGCGA |
| siRNA 2775 | 2775 | CGCAGGGUCAGGCCUGCCC | 9129 | GGGCAGGCCUGACCCUGCG |
| siRNA 2776 | 2776 | GCAGGGUCAGGCCUGCCCU | 9130 | AGGGCAGGCCUGACCCUGC |
| siRNA 2777 | 2777 | CAGGGUCAGGCCUGCCCUG | 9131 | CAGGGCAGGCCUGACCCUG |
| siRNA 2778 | 2778 | AGGGUCAGGCCUGCCCUGU | 9132 | ACAGGGCAGGCCUGACCCU |
| siRNA 2779 | 2779 | GGGUCAGGCCUGCCCUGUC | 9133 | GACAGGGCAGGCCUGACCC |
| siRNA 2780 | 2780 | GGUCAGGCCUGCCCUGUCC | 9134 | GGACAGGGCAGGCCUGACC |
| siRNA 2781 | 2781 | GUCAGGCCUGCCCUGUCCC | 9135 | GGGACAGGGCAGGCCUGAC |
| siRNA 2782 | 2782 | UCAGGCCUGCCCUGUCCCG | 9136 | CGGGACAGGGCAGGCCUGA |
| siRNA 2783 | 2783 | CAGGCCUGCCCUGUCCCGA | 9137 | UCGGGACAGGGCAGGCCUG |
| siRNA 2784 | 2784 | AGGCCUGCCCUGUCCCGAA | 9138 | UUCGGGACAGGGCAGGCCU |
| siRNA 2785 | 2785 | GGCCUGCCCUGUCCCGAAG | 9139 | CUUCGGGACAGGGCAGGCC |
| siRNA 2786 | 2786 | GCCUGCCCUGUCCCGAAGU | 9140 | ACUUCGGGACAGGGCAGGC |
| siRNA 2787 | 2787 | CCUGCCCUGUCCCGAAGUG | 9141 | CACUUCGGGACAGGGCAGG |
| siRNA 2788 | 2788 | CUGCCCUGUCCCGAAGUGA | 9142 | UCACUUCGGGACAGGGCAG |
| siRNA 2789 | 2789 | UGCCCUGUCCCGAAGUGAU | 9143 | AUCACUUCGGGACAGGGCA |
| siRNA 2790 | 2790 | GCCCUGUCCCGAAGUGAUC | 9144 | GAUCACUUCGGGACAGGGC |
| siRNA 2791 | 2791 | CCCUGUCCCGAAGUGAUCU | 9145 | AGAUCACUUCGGGACAGGG |
| siRNA 2792 | 2792 | CCUGUCCCGAAGUGAUCUC | 9146 | GAGAUCACUUCGGGACAGG |
| siRNA 2793 | 2793 | CUGUCCCGAAGUGAUCUCC | 9147 | GGAGAUCACUUCGGGACAG |
| siRNA 2794 | 2794 | UGUCCCGAAGUGAUCUCCU | 9148 | AGGAGAUCACUUCGGGACA |
| siRNA 2795 | 2795 | GUCCCGAAGUGAUCUCCUG | 9149 | CAGGAGAUCACUUCGGGAC |
| siRNA 2796 | 2796 | UCCCGAAGUGAUCUCCUGG | 9150 | CCAGGAGAUCACUUCGGGA |
| siRNA 2797 | 2797 | CCCGAAGUGAUCUCCUGGA | 9151 | UCCAGGAGAUCACUUCGGG |
| siRNA 2798 | 2798 | CCGAAGUGAUCUCCUGGAA | 9152 | UUCCAGGAGAUCACUUCGG |
| siRNA 2799 | 2799 | CGAAGUGAUCUCCUGGAAG | 9153 | CUUCCAGGAGAUCACUUCG |
| siRNA 2800 | 2800 | GAAGUGAUCUCCUGGAAG | 9154 | UCUUCCAGGAGAUCACUUC |
| | | A | | |
| siRNA 2801 | 2801 | AAGUGAUCUCCUGGAAGAC | 9155 | GUCUUCCAGGAGAUCACUU |
| siRNA 2802 | 2802 | AGUGAUCUCCUGGAAGACA | 9156 | UGUCUUCCAGGAGAUCACU |
| siRNA 2803 | 2803 | GUGAUCUCCUGGAAGACAA | 9157 | UUGUCUUCCAGGAGAUCAC |
| siRNA 2804 | 2804 | UGAUCUCCUGGAAGACAAG | 9158 | CUUGUCUUCCAGGAGAUCA |
| siRNA 2805 | 2805 | GAUCUCCUGGAAGACAAGU | 9159 | ACUUGUCUUCCAGGAGAUC |
| siRNA 2806 | 2806 | AUCUCCUGGAAGACAAGUG | 9160 | CACUUGUCUUCCAGGAGAU |
| siRNA 2807 | 2807 | UCUCCUGGAAGACAAGUGC | 9161 | GCACUUGUCUUCCAGGAGA |
| siRNA 2808 | 2808 | CUCCUGGAAGACAAGUGCC | 9162 | GGCACUUGUCUUCCAGGAG |
| siRNA 2809 | 2809 | UCCUGGAAGACAAGUGCCU | 9163 | AGGCACUUGUCUUCCAGGA |
| siRNA 2810 | 2810 | CCUGGAAGACAAGUGCCUU | 9164 | AAGGCACUUGUCUUCCAGG |
| siRNA 2811 | 2811 | CUGGAAGACAAGUGCCUUC | 9165 | GAAGGCACUUGUCUUCCAG |
| siRNA 2812 | 2812 | UGGAAGACAAGUGCCUUCU | 9166 | AGAAGGCACUUGUCUUCCA |
| siRNA 2813 | 2813 | GGAAGACAAGUGCCUUCUC | 9167 | GAGAAGGCACUUGUCUUCC |
| siRNA 2814 | 2814 | GAAGACAAGUGCCUUCUCC | 9168 | GGAGAAGGCACUUGUCUUC |
| siRNA 2815 | 2815 | AAGACAAGUGCCUUCUCCC | 9169 | GGGAGAAGGCACUUGUCU |
| | | | | U |
| siRNA 2816 | 2816 | AGACAAGUGCCUUCUCCCU | 9170 | AGGGAGAAGGCACUUGUC |
| | | | | U |
| siRNA 2817 | 2817 | GACAAGUGCCUUCUCCCUC | 9171 | GAGGGAGAAGGCACUUGU |
| | | | | C |
| siRNA 2818 | 2818 | ACAAGUGCCUUCUCCCUCC | 9172 | GGAGGGAGAAGGCACUUG |
| | | | | U |
| siRNA 2819 | 2819 | CAAGUGCCUUCUCCCUCCA | 9173 | UGGAGGGAGAAGGCACUU |
| | | | | G |
| siRNA 2820 | 2820 | AAGUGCCUUCUCCCUCCAU | 9174 | AUGGAGGGAGAAGGCACU |
| | | | | U |
| siRNA 2821 | 2821 | AGUGCCUUCUCCCUCCAUG | 9175 | CAUGGAGGGAGAAGGCAC |
| | | | | U |
| siRNA 2822 | 2822 | GUGCCUUCUCCCUCCAUGG | 9176 | CCAUGGAGGGAGAAGGCAC |
| siRNA 2823 | 2823 | UGCCUUCUCCCUCCAUGGA | 9177 | UCCAUGGAGGGAGAAGGC |
| | | | | A |
| siRNA 2824 | 2824 | GCCUUCUCCCUCCAUGGAU | 9178 | AUCCAUGGAGGGAGAAGG |
| | | | | C |
| siRNA 2825 | 2825 | CCUUCUCCCUCCAUGGAUC | 9179 | GAUCCAUGGAGGGAGAAG |
| | | | | G |
| siRNA 2826 | 2826 | CUUCUCCCUCCAUGGAUCU | 9180 | AGAUCCAUGGAGGGAGAA |
| | | | | G |
| siRNA 2827 | 2827 | UUCUCCCUCCAUGGAUCUG | 9181 | CAGAUCCAUGGAGGGAGA |
| | | | | A |
| siRNA 2828 | 2828 | UCUCCCUCCAUGGAUCUGU | 9182 | ACAGAUCCAUGGAGGGAG |
| | | | | A |
| siRNA 2829 | 2829 | CUCCCUCCAUGGAUCUGUG | 9183 | CACAGAUCCAUGGAGGGAG |
| siRNA 2830 | 2830 | UCCCUCCAUGGAUCUGUGA | 9184 | UCACAGAUCCAUGGAGGGA |
| siRNA 2831 | 2831 | CCCUCCAUGGAUCUGUGAU | 9185 | AUCACAGAUCCAUGGAGGG |
| siRNA 2832 | 2832 | CCUCCAUGGAUCUGUGAUC | 9186 | GAUCACAGAUCCAUGGAGG |
| siRNA 2833 | 2833 | CUCCAUGGAUCUGUGAUCU | 9187 | AGAUCACAGAUCCAUGGAG |
| siRNA 2834 | 2834 | UCCAUGGAUCUGUGAUCUU | 9188 | AAGAUCACAGAUCCAUGGA |
| siRNA 2835 | 2835 | CCAUGGAUCUGUGAUCUUC | 9189 | GAAGAUCACAGAUCCAUGG |
| siRNA 2836 | 2836 | CAUGGAUCUGUGAUCUUCC | 9190 | GGAAGAUCACAGAUCCAUG |
| siRNA 2837 | 2837 | AUGGAUCUGUGAUCUUCCC | 9191 | GGGAAGAUCACAGAUCCAU |
| siRNA 2838 | 2838 | UGGAUCUGUGAUCUUCCCA | 9192 | UGGGAAGAUCACAGAUCCA |
| siRNA 2839 | 2839 | GGAUCUGUGAUCUUCCCAG | 9193 | CUGGGAAGAUCACAGAUCC |
| siRNA 2840 | 2840 | GAUCUGUGAUCUUCCCAGC | 9194 | GCUGGGAAGAUCACAGAUC |
| siRNA 2841 | 2841 | AUCUGUGAUCUUCCCAGCU | 9195 | AGCUGGGAAGAUCACAGA |
| | | | | U |
| siRNA 2842 | 2842 | UCUGUGAUCUUCCCAGCUC | 9196 | GAGCUGGGAAGAUCACAG |
| | | | | A |
| siRNA 2843 | 2843 | CUGUGAUCUUCCCAGCUCU | 9197 | AGAGCUGGGAAGAUCACA |
| | | | | G |
| siRNA 2844 | 2844 | UGUGAUCUUCCCAGCUCUG | 9198 | CAGAGCUGGGAAGAUCACA |
| siRNA 2845 | 2845 | GUGAUCUUCCCAGCUCUGC | 9199 | GCAGAGCUGGGAAGAUCAC |
| siRNA 2846 | 2846 | UGAUCUUCCCAGCUCUGCA | 9200 | UGCAGAGCUGGGAAGAUC |
| | | | | A |
| siRNA 2847 | 2847 | GAUCUUCCCAGCUCUGCAU | 9201 | AUGCAGAGCUGGGAAGAU |
| | | | | C |
| siRNA 2848 | 2848 | AUCUUCCCAGCUCUGCAUC | 9202 | GAUGCAGAGCUGGGAAGA |
| | | | | U |
| siRNA 2849 | 2849 | UCUUCCCAGCUCUGCAUCA | 9203 | UGAUGCAGAGCUGGGAAG |
| | | | | A |
| siRNA 2850 | 2850 | CUUCCCAGCUCUGCAUCAA | 9204 | UUGAUGCAGAGCUGGGAA |
| | | | | G |
| siRNA 2851 | 2851 | UUCCCAGCUCUGCAUCAAC | 9205 | GUUGAUGCAGAGCUGGGA |
| | | | | A |
| siRNA 2852 | 2852 | UCCCAGCUCUGCAUCAACA | 9206 | UGUUGAUGCAGAGCUGGG |
| | | | | A |
| siRNA 2853 | 2853 | CCCAGCUCUGCAUCAACAC | 9207 | GUGUUGAUGCAGAGCUGG |
| | | | | G |
| siRNA 2854 | 2854 | CCAGCUCUGCAUCAACACA | 9208 | UGUGUUGAUGCAGAGCUG |
| | | | | G |
| siRNA 2855 | 2855 | CAGCUCUGCAUCAACACAG | 9209 | CUGUGUUGAUGCAGAGCU |
| | | | | G |
| siRNA 2856 | 2856 | AGCUCUGCAUCAACACAGC | 9210 | GCUGUGUUGAUGCAGAGC |
| | | | | U |
| siRNA 2857 | 2857 | GCUCUGCAUCAACACAGCA | 9211 | UGCUGUGUUGAUGCAGAG |
| | | | | C |
| siRNA 2858 | 2858 | CUCUGCAUCAACACAGCAG | 9212 | CUGCUGUGUUGAUGCAGA |
| | | | | G |
| siRNA 2859 | 2859 | UCUGCAUCAACACAGCAGC | 9213 | GCUGCUGUGUUGAUGCAG |
| | | | | A |
| siRNA 2860 | 2860 | CUGCAUCAACACAGCAGCC | 9214 | GGCUGCUGUGUUGAUGCA |
| | | | | G |
| siRNA 2861 | 2861 | UGCAUCAACACAGCAGCCU | 9215 | AGGCUGCUGUGUUGAUGC |
| | | | | A |
| siRNA 2862 | 2862 | GCAUCAACACAGCAGCCUG | 9216 | CAGGCUGCUGUGUUGAUGC |
| siRNA 2863 | 2863 | CAUCAACACAGCAGCCUGC | 9217 | GCAGGCUGCUGUGUUGAU |
| | | | | G |
| siRNA 2864 | 2864 | AUCAACACAGCAGCCUGCA | 9218 | UGCAGGCUGCUGUGUUGA |
| | | | | U |
| siRNA 2865 | 2865 | UCAACACAGCAGCCUGCAG | 9219 | CUGCAGGCUGCUGUGUUGA |
| siRNA 2866 | 2866 | CAACACAGCAGCCUGCAGA | 9220 | UCUGCAGGCUGCUGUGUUG |
| siRNA 2867 | 2867 | AACACAGCAGCCUGCAGAU | 9221 | AUCUGCAGGCUGCUGUGUU |
| siRNA 2868 | 2868 | ACACAGCAGCCUGCAGAUA | 9222 | UAUCUGCAGGCUGCUGUGU |
| siRNA 2869 | 2869 | CACAGCAGCCUGCAGAUAA | 9223 | UUAUCUGCAGGCUGCUGUG |
| siRNA 2870 | 2870 | ACAGCAGCCUGCAGAUAAC | 9224 | GUUAUCUGCAGGCUGCUGU |
| siRNA 2871 | 2871 | CAGCAGCCUGCAGAUAACA | 9225 | UGUUAUCUGCAGGCUGCUG |
| siRNA 2872 | 2872 | AGCAGCCUGCAGAUAACAC | 9226 | GUGUUAUCUGCAGGCUGCU |
| siRNA 2873 | 2873 | GCAGCCUGCAGAUAACACU | 9227 | AGUGUUAUCUGCAGGCUGC |
| siRNA 2874 | 2874 | CAGCCUGCAGAUAACACUU | 9228 | AAGUGUUAUCUGCAGGCU |
| | | | | G |
| siRNA 2875 | 2875 | AGCCUGCAGAUAACACUUG | 9229 | CAAGUGUUAUCUGCAGGCU |
| siRNA 2876 | 2876 | GCCUGCAGAUAACACUUGG | 9230 | CCAAGUGUUAUCUGCAGGC |
| siRNA 2877 | 2877 | CCUGCAGAUAACACUUGGG | 9231 | CCCAAGUGUUAUCUGCAGG |
| siRNA 2878 | 2878 | CUGCAGAUAACACUUGGGG | 9232 | CCCCAAGUGUUAUCUGCAG |
| siRNA 2879 | 2879 | UGCAGAUAACACUUGGGG | 9233 | CCCCCAAGUGUUAUCUGCA |
| | | G | | |
| siRNA 2880 | 2880 | GCAGAUAACACUUGGGGG | 9234 | CCCCCCAAGUGUUAUCUGC |
| | | G | | |
| siRNA 2881 | 2881 | CAGAUAACACUUGGGGGG | 9235 | UCCCCCCAAGUGUUAUCUG |
| | | A | | |
| siRNA 2882 | 2882 | AGAUAACACUUGGGGGGA | 9236 | GUCCCCCCAAGUGUUAUCU |
| | | C | | |
| siRNA 2883 | 2883 | GAUAACACUUGGGGGGACC | 9237 | GGUCCCCCCAAGUGUUAUC |
| siRNA 2884 | 2884 | AUAACACUUGGGGGGACCU | 9238 | AGGUCCCCCCAAGUGUUAU |
| siRNA 2885 | 2885 | UAACACUUGGGGGGACCUC | 9239 | GAGGUCCCCCCAAGUGUUA |
| siRNA 2886 | 2886 | AACACUUGGGGGGACCUCA | 9240 | UGAGGUCCCCCCAAGUGUU |
| siRNA 2887 | 2887 | ACACUUGGGGGGACCUCAG | 9241 | CUGAGGUCCCCCCAAGUGU |
| siRNA 2888 | 2888 | CACUUGGGGGGACCUCAGC | 9242 | GCUGAGGUCCCCCCAAGUG |
| siRNA 2889 | 2889 | ACUUGGGGGGACCUCAGCC | 9243 | GGCUGAGGUCCCCCCAAGU |
| siRNA 2890 | 2890 | CUUGGGGGGACCUCAGCCU | 9244 | AGGCUGAGGUCCCCCCAAG |
| siRNA 2891 | 2891 | UUGGGGGGACCUCAGCCUC | 9245 | GAGGCUGAGGUCCCCCCAA |
| siRNA 2892 | 2892 | UGGGGGGACCUCAGCCUCU | 9246 | AGAGGCUGAGGUCCCCCCA |
| siRNA 2893 | 2893 | GGGGGGACCUCAGCCUCUA | 9247 | UAGAGGCUGAGGUCCCCCC |
| siRNA 2894 | 2894 | GGGGGACCUCAGCCUCUAU | 9248 | AUAGAGGCUGAGGUCCCCC |
| siRNA 2895 | 2895 | GGGGACCUCAGCCUCUAUU | 9249 | AAUAGAGGCUGAGGUCCCC |
| siRNA 2896 | 2896 | GGGACCUCAGCCUCUAUUC | 9250 | GAAUAGAGGCUGAGGUCCC |
| siRNA 2897 | 2897 | GGACCUCAGCCUCUAUUCG | 9251 | CGAAUAGAGGCUGAGGUCC |
| siRNA 2898 | 2898 | GACCUCAGCCUCUAUUCGC | 9252 | GCGAAUAGAGGCUGAGGU |
| | | | | C |
| siRNA 2899 | 2899 | ACCUCAGCCUCUAUUCGCA | 9253 | UGCGAAUAGAGGCUGAGG |
| | | | | U |
| siRNA 2900 | 2900 | CCUCAGCCUCUAUUCGCAA | 9254 | UUGCGAAUAGAGGCUGAG |
| | | | | G |
| siRNA 2901 | 2901 | CUCAGCCUCUAUUCGCAAC | 9255 | GUUGCGAAUAGAGGCUGA |
| | | | | G |
| siRNA 2902 | 2902 | UCAGCCUCUAUUCGCAACU | 9256 | AGUUGCGAAUAGAGGCUG |
| | | | | A |
| siRNA 2903 | 2903 | CAGCCUCUAUUCGCAACUC | 9257 | GAGUUGCGAAUAGAGGCU |
| | | | | G |
| siRNA 2904 | 2904 | AGCCUCUAUUCGCAACUCA | 9258 | UGAGUUGCGAAUAGAGGC |
| | | | | U |
| siRNA 2905 | 2905 | GCCUCUAUUCGCAACUCAU | 9259 | AUGAGUUGCGAAUAGAGG |
| | | | | C |
| siRNA 2906 | 2906 | CCUCUAUUCGCAACUCAUA | 9260 | UAUGAGUUGCGAAUAGAG |
| | | | | G |
| siRNA 2907 | 2907 | CUCUAUUCGCAACUCAUAA | 9261 | UUAUGAGUUGCGAAUAGA |
| | | | | G |
| siRNA 2908 | 2908 | UCUAUUCGCAACUCAUAAU | 9262 | AUUAUGAGUUGCGAAUAG |
| | | | | A |
| siRNA 2909 | 2909 | CUAUUCGCAACUCAUAAUC | 9263 | GAUUAUGAGUUGCGAAUA |
| | | | | G |
| siRNA 2910 | 2910 | UAUUCGCAACUCAUAAUCC | 9264 | GGAUUAUGAGUUGCGAAU |
| | | | | A |
| siRNA 2911 | 2911 | AUUCGCAACUCAUAAUCCG | 9265 | CGGAUUAUGAGUUGCGAA |
| | | | | U |
| siRNA 2912 | 2912 | UUCGCAACUCAUAAUCCGU | 9266 | ACGGAUUAUGAGUUGCGA |
| | | | | A |
| siRNA 2913 | 2913 | UCGCAACUCAUAAUCCGUA | 9267 | UACGGAUUAUGAGUUGCG |
| | | | | A |
| siRNA 2914 | 2914 | CGCAACUCAUAAUCCGUAG | 9268 | CUACGGAUUAUGAGUUGC |
| | | | | G |
| siRNA 2915 | 2915 | GCAACUCAUAAUCCGUAGA | 9269 | UCUACGGAUUAUGAGUUG |
| | | | | C |
| siRNA 2916 | 2916 | CAACUCAUAAUCCGUAGAC | 9270 | GUCUACGGAUUAUGAGUU |
| | | | | G |
| siRNA 2917 | 2917 | AACUCAUAAUCCGUAGACU | 9271 | AGUCUACGGAUUAUGAGU |
| | | | | U |
| siRNA 2918 | 2918 | ACUCAUAAUCCGUAGACUA | 9272 | UAGUCUACGGAUUAUGAG |
| | | | | U |
| siRNA 2919 | 2919 | CUCAUAAUCCGUAGACUAC | 9273 | GUAGUCUACGGAUUAUGA |
| | | | | G |
| siRNA 2920 | 2920 | UCAUAAUCCGUAGACUACA | 9274 | UGUAGUCUACGGAUUAUG |
| | | | | A |
| siRNA 2921 | 2921 | CAUAAUCCGUAGACUACAA | 9275 | UUGUAGUCUACGGAUUAU |
| | | | | G |
| siRNA 2922 | 2922 | AUAAUCCGUAGACUACAAG | 9276 | CUUGUAGUCUACGGAUUA |
| | | | | U |
| siRNA 2923 | 2923 | UAAUCCGUAGACUACAAGA | 9277 | UCUUGUAGUCUACGGAUU |
| | | | | A |
| siRNA 2924 | 2924 | AAUCCGUAGACUACAAGAU | 9278 | AUCUUGUAGUCUACGGAU |
| | | | | U |
| siRNA 2925 | 2925 | AUCCGUAGACUACAAGAUG | 9279 | CAUCUUGUAGUCUACGGAU |
| siRNA 2926 | 2926 | UCCGUAGACUACAAGAUGA | 9280 | UCAUCUUGUAGUCUACGGA |
| siRNA 2927 | 2927 | CCGUAGACUACAAGAUGAA | 9281 | UUCAUCUUGUAGUCUACGG |
| siRNA 2928 | 2928 | CGUAGACUACAAGAUGAA | 9282 | UUUCAUCUUGUAGUCUACG |
| | | A | | |
| siRNA 2929 | 2929 | GUAGACUACAAGAUGAAA | 9283 | AUUUCAUCUUGUAGUCUAC |
| | | U | | |
| siRNA 2930 | 2930 | UAGACUACAAGAUGAAAU | 9284 | GAUUUCAUCUUGUAGUCU |
| | | C | | A |
| siRNA 2931 | 2931 | AGACUACAAGAUGAAAUC | 9285 | AGAUUUCAUCUUGUAGUC |
| | | U | | U |
| siRNA 2932 | 2932 | GACUACAAGAUGAAAUCUC | 9286 | GAGAUUUCAUCUUGUAGU |
| | | | | C |
| siRNA 2933 | 2933 | ACUACAAGAUGAAAUCUCA | 9287 | UGAGAUUUCAUCUUGUAG |
| | | | | U |
| siRNA 2934 | 2934 | CUACAAGAUGAAAUCUCAA | 9288 | UUGAGAUUUCAUCUUGUA |
| | | | | G |
| siRNA 2935 | 2935 | UACAAGAUGAAAUCUCAA | 9289 | AUUGAGAUUUCAUCUUGU |
| | | U | | A |
| siRNA 2936 | 2936 | ACAAGAUGAAAUCUCAAU | 9290 | UAUUGAGAUUUCAUCUUG |
| | | A | | U |
| siRNA 2937 | 2937 | CAAGAUGAAAUCUCAAUA | 9291 | UUAUUGAGAUUUCAUCUU |
| | | A | | G |
| siRNA 2938 | 2938 | AAGAUGAAAUCUCAAUAA | 9292 | UUUAUUGAGAUUUCAUCU |
| | | A | | U |
| siRNA 2939 | 2939 | AGAUGAAAUCUCAAUAAA | 9293 | AUUUAUUGAGAUUUCAUC |
| | | U | | U |
| siRNA 2940 | 2940 | GAUGAAAUCUCAAUAAAU | 9294 | AAUUUAUUGAGAUUUCAU |
| | | U | | C |
| siRNA 2941 | 2941 | AUGAAAUCUCAAUAAAUU | 9295 | UAAUUUAUUGAGAUUUCA |
| | | A | | U |
| siRNA 2942 | 2942 | UGAAAUCUCAAUAAAUUA | 9296 | AUAAUUUAUUGAGAUUUC |
| | | U | | A |
| siRNA 2943 | 2943 | GAAAUCUCAAUAAAUUAU | 9297 | AAUAAUUUAUUGAGAUUU |
| | | U | | C |
| siRNA 2944 | 2944 | AAAUCUCAAUAAAUUAUU | 9298 | AAAUAAUUUAUUGAGAUU |
| | | U | | U |
| siRNA 2945 | 2945 | AAUCUCAAUAAAUUAUUU | 9299 | AAAAUAAUUUAUUGAGAU |
| | | U | | U |
| siRNA 2946 | 2946 | AUCUCAAUAAAUUAUUUU | 9300 | AAAAAUAAUUUAUUGAGA |
| | | U | | U |
| siRNA 2947 | 2947 | UCUCAAUAAAUUAUUUUU | 9301 | CAAAAAUAAUUUAUUGAG |
| | | G | | A |
| siRNA 2948 | 2948 | CUCAAUAAAUUAUUUUUG | 9302 | UCAAAAAUAAUUUAUUGA |
| | | A | | G |
| siRNA 2949 | 2949 | UCAAUAAAUUAUUUUUGA | 9303 | CUCAAAAAUAAUUUAUUG |
| | | G | | A |
| siRNA 2950 | 2950 | CAAUAAAUUAUUUUUGAG | 9304 | ACUCAAAAAUAAUUUAUU |
| | | U | | G |
| siRNA 2951 | 2951 | AAUAAAUUAUUUUUGAGU | 9305 | AACUCAAAAAUAAUUUAU |
| | | U | | U |
| siRNA 2952 | 2952 | AUAAAUUAUUUUUGAGUU | 9306 | AAACUCAAAAAUAAUUUA |
| | | U | | U |
| siRNA 2953 | 2953 | UAAAUUAUUUUUGAGUUU | 9307 | UAAACUCAAAAAUAAUUU |
| | | A | | A |
| siRNA 2954 | 2954 | AAAUUAUUUUUGAGUUUA | 9308 | AUAAACUCAAAAAUAAUU |
| | | U | | U |
| siRNA 2955 | 2955 | AAUUAUUUUUGAGUUUAU | 9309 | AAUAAACUCAAAAAUAAU |
| | | U | | U |
| siRNA 2956 | 2956 | AUUAUUUUUGAGUUUAUU | 9310 | UAAUAAACUCAAAAAUAA |
| | | A | | U |
| siRNA 2957 | 2957 | UUAUUUUUGAGUUUAUUA | 9311 | UUAAUAAACUCAAAAAUA |
| | | A | | A |
| siRNA 2958 | 2958 | UAUUUUUGAGUUUAUUAA | 9312 | UUUAAUAAACUCAAAAAU |
| | | A | | A |
| siRNA 2959 | 2959 | AUUUUUGAGUUUAUUAAA | 9313 | CUUUAAUAAACUCAAAAA |
| | | G | | U |
| siRNA 2960 | 2960 | UUUUUGAGUUUAUUAAAG | 9314 | UCUUUAAUAAACUCAAAA |
| | | A | | A |
| siRNA 2961 | 2961 | UUUUGAGUUUAUUAAAGA | 9315 | AUCUUUAAUAAACUCAAA |
| | | U | | A |
| siRNA 2962 | 2962 | UUUGAGUUUAUUAAAGAU | 9316 | AAUCUUUAAUAAACUCAA |
| | | U | | A |
| siRNA 2963 | 2963 | UUGAGUUUAUUAAAGAUU | 9317 | CAAUCUUUAAUAAACUCAA |
| | | G | | |
| siRNA 2964 | 2964 | UGAGUUUAUUAAAGAUUG | 9318 | UCAAUCUUUAAUAAACUCA |
| | | A | | |
| siRNA 2965 | 2965 | GAGUUUAUUAAAGAUUGA | 9319 | GUCAAUCUUUAAUAAACUC |
| | | C | | |
| siRNA 2966 | 2966 | AGUUUAUUAAAGAUUGAC | 9320 | UGUCAAUCUUUAAUAAAC |
| | | A | | U |
| siRNA 2967 | 2967 | GUUUAUUAAAGAUUGACA | 9321 | AUGUCAAUCUUUAAUAAA |
| | | U | | C |
| siRNA 2968 | 2968 | UUUAUUAAAGAUUGACAU | 9322 | AAUGUCAAUCUUUAAUAA |
| | | U | | A |
| siRNA 2969 | 2969 | UUAUUAAAGAUUGACAUU | 9323 | AAAUGUCAAUCUUUAAUA |
| | | U | | A |
| siRNA 2970 | 2970 | UAUUAAAGAUUGACAUUU | 9324 | AAAAUGUCAAUCUUUAAU |
| | | U | | A |
| siRNA 2971 | 2971 | AUUAAAGAUUGACAUUUU | 9325 | UAAAAUGUCAAUCUUUAA |
| | | A | | U |
| siRNA 2972 | 2972 | UUAAAGAUUGACAUUUUA | 9326 | UUAAAAUGUCAAUCUUUA |
| | | A | | A |
| siRNA 2973 | 2973 | UAAAGAUUGACAUUUUAA | 9327 | CUUAAAAUGUCAAUCUUU |
| | | G | | A |
| siRNA 2974 | 2974 | AAAGAUUGACAUUUUAAG | 9328 | ACUUAAAAUGUCAAUCUU |
| | | U | | U |
| siRNA 2975 | 2975 | AAGAUUGACAUUUUAAGU | 9329 | UACUUAAAAUGUCAAUCU |
| | | A | | U |
| siRNA 2976 | 2976 | AGAUUGACAUUUUAAGUA | 9330 | GUACUUAAAAUGUCAAUC |
| | | C | | U |
| siRNA 2977 | 2977 | GAUUGACAUUUUAAGUAC | 9331 | UGUACUUAAAAUGUCAAU |
| | | A | | C |
| siRNA 2978 | 2978 | AUUGACAUUUUAAGUACA | 9332 | UUGUACUUAAAAUGUCAA |
| | | A | | U |
| siRNA 2979 | 2979 | UUGACAUUUUAAGUACAA | 9333 | GUUGUACUUAAAAUGUCA |
| | | C | | A |
| siRNA 2980 | 2980 | UGACAUUUUAAGUACAAC | 9334 | AGUUGUACUUAAAAUGUC |
| | | U | | A |
| siRNA 2981 | 2981 | GACAUUUUAAGUACAACU | 9335 | AAGUUGUACUUAAAAUGU |
| | | U | | C |
| siRNA 2982 | 2982 | ACAUUUUAAGUACAACUU | 9336 | AAAGUUGUACUUAAAAUG |
| | | U | | U |
| siRNA 2983 | 2983 | CAUUUUAAGUACAACUUU | 9337 | AAAAGUUGUACUUAAAAU |
| | | U | | G |
| siRNA 2984 | 2984 | AUUUUAAGUACAACUUUU | 9338 | UAAAAGUUGUACUUAAAA |
| | | A | | U |
| siRNA 2985 | 2985 | UUUUAAGUACAACUUUUA | 9339 | UUAAAAGUUGUACUUAAA |
| | | A | | A |
| siRNA 2986 | 2986 | UUUAAGUACAACUUUUAA | 9340 | CUUAAAAGUUGUACUUAA |
| | | G | | A |
| siRNA 2987 | 2987 | UUAAGUACAACUUUUAAG | 9341 | CCUUAAAAGUUGUACUUA |
| | | G | | A |
| siRNA 2988 | 2988 | UAAGUACAACUUUUAAGG | 9342 | UCCUUAAAAGUUGUACUU |
| | | A | | A |
| siRNA 2989 | 2989 | AAGUACAACUUUUAAGGA | 9343 | GUCCUUAAAAGUUGUACU |
| | | C | | U |
| siRNA 2990 | 2990 | AGUACAACUUUUAAGGAC | 9344 | AGUCCUUAAAAGUUGUAC |
| | | U | | U |
| siRNA 2991 | 2991 | GUACAACUUUUAAGGACU | 9345 | UAGUCCUUAAAAGUUGUA |
| | | A | | C |
| siRNA 2992 | 2992 | UACAACUUUUAAGGACUA | 9346 | UUAGUCCUUAAAAGUUGU |
| | | A | | A |
| siRNA 2993 | 2993 | ACAACUUUUAAGGACUAA | 9347 | AUUAGUCCUUAAAAGUUG |
| | | U | | U |
| siRNA 2994 | 2994 | CAACUUUUAAGGACUAAU | 9348 | AAUUAGUCCUUAAAAGUU |
| | | U | | G |
| siRNA 2995 | 2995 | AACUUUUAAGGACUAAUU | 9349 | UAAUUAGUCCUUAAAAGU |
| | | A | | U |
| siRNA 2996 | 2996 | ACUUUUAAGGACUAAUUA | 9350 | GUAAUUAGUCCUUAAAAG |
| | | C | | U |
| siRNA 2997 | 2997 | CUUUUAAGGACUAAUUAC | 9351 | AGUAAUUAGUCCUUAAAA |
| | | U | | G |
| siRNA 2998 | 2998 | UUUUAAGGACUAAUUACU | 9352 | CAGUAAUUAGUCCUUAAA |
| | | G | | A |
| siRNA 2999 | 2999 | UUUAAGGACUAAUUACUG | 9353 | ACAGUAAUUAGUCCUUAA |
| | | U | | A |
| siRNA 3000 | 3000 | UUAAGGACUAAUUACUGU | 9354 | CACAGUAAUUAGUCCUUAA |
| | | G | | |
| siRNA 3001 | 3001 | UAAGGACUAAUUACUGUG | 9355 | UCACAGUAAUUAGUCCUUA |
| | | A | | |
| siRNA 3002 | 3002 | AAGGACUAAUUACUGUGA | 9356 | AUCACAGUAAUUAGUCCUU |
| | | U | | |
| siRNA 3003 | 3003 | AGGACUAAUUACUGUGAU | 9357 | CAUCACAGUAAUUAGUCCU |
| | | G | | |
| siRNA 3004 | 3004 | GGACUAAUUACUGUGAUG | 9358 | CCAUCACAGUAAUUAGUCC |
| | | G | | |
| siRNA 3005 | 3005 | GACUAAUUACUGUGAUGG | 9359 | UCCAUCACAGUAAUUAGUC |
| | | A | | |
| siRNA 3006 | 3006 | ACUAAUUACUGUGAUGGA | 9360 | GUCCAUCACAGUAAUUAGU |
| | | C | | |
| siRNA 3007 | 3007 | CUAAUUACUGUGAUGGAC | 9361 | UGUCCAUCACAGUAAUUAG |
| | | A | | |
| siRNA 3008 | 3008 | UAAUUACUGUGAUGGACA | 9362 | GUGUCCAUCACAGUAAUUA |
| | | C | | |
| siRNA 3009 | 3009 | AAUUACUGUGAUGGACAC | 9363 | UGUGUCCAUCACAGUAAUU |
| | | A | | |
| siRNA 3010 | 3010 | AUUACUGUGAUGGACACA | 9364 | CUGUGUCCAUCACAGUAAU |
| | | G | | |
| siRNA 3011 | 3011 | UUACUGUGAUGGACACAG | 9365 | UCUGUGUCCAUCACAGUAA |
| | | A | | |
| siRNA 3012 | 3012 | UACUGUGAUGGACACAGA | 9366 | UUCUGUGUCCAUCACAGUA |
| | | A | | |
| siRNA 3013 | 3013 | ACUGUGAUGGACACAGAA | 9367 | UUUCUGUGUCCAUCACAGU |
| | | A | | |
| siRNA 3014 | 3014 | CUGUGAUGGACACAGAAA | 9368 | AUUUCUGUGUCCAUCACAG |
| | | U | | |
| siRNA 3015 | 3015 | UGUGAUGGACACAGAAAU | 9369 | CAUUUCUGUGUCCAUCACA |
| | | G | | |
| siRNA 3016 | 3016 | GUGAUGGACACAGAAAUG | 9370 | ACAUUUCUGUGUCCAUCAC |
| | | U | | |
| siRNA 3017 | 3017 | UGAUGGACACAGAAAUGU | 9371 | UACAUUUCUGUGUCCAUCA |
| | | A | | |
| siRNA 3018 | 3018 | GAUGGACACAGAAAUGUA | 9372 | CUACAUUUCUGUGUCCAUC |
| | | G | | |
| siRNA 3019 | 3019 | AUGGACACAGAAAUGUAG | 9373 | GCUACAUUUCUGUGUCCAU |
| | | C | | |
| siRNA 3020 | 3020 | UGGACACAGAAAUGUAGC | 9374 | AGCUACAUUUCUGUGUCCA |
| | | U | | |
| siRNA 3021 | 3021 | GGACACAGAAAUGUAGCU | 9375 | CAGCUACAUUUCUGUGUCC |
| | | G | | |
| siRNA 3022 | 3022 | GACACAGAAAUGUAGCUG | 9376 | ACAGCUACAUUUCUGUGUC |
| | | U | | |
| siRNA 3023 | 3023 | ACACAGAAAUGUAGCUGU | 9377 | CACAGCUACAUUUCUGUGU |
| | | G | | |
| siRNA 3024 | 3024 | CACAGAAAUGUAGCUGUG | 9378 | ACACAGCUACAUUUCUGUG |
| | | U | | |
| siRNA 3025 | 3025 | ACAGAAAUGUAGCUGUGU | 9379 | AACACAGCUACAUUUCUGU |
| | | U | | |
| siRNA 3026 | 3026 | CAGAAAUGUAGCUGUGUU | 9380 | GAACACAGCUACAUUUCUG |
| | | C | | |
| siRNA 3027 | 3027 | AGAAAUGUAGCUGUGUUC | 9381 | AGAACACAGCUACAUUUCU |
| | | U | | |
| siRNA 3028 | 3028 | GAAAUGUAGCUGUGUUCU | 9382 | CAGAACACAGCUACAUUUC |
| | | G | | |
| siRNA 3029 | 3029 | AAAUGUAGCUGUGUUCUG | 9383 | CCAGAACACAGCUACAUUU |
| | | G | | |
| siRNA 3030 | 3030 | AAUGUAGCUGUGUUCUGG | 9384 | UCCAGAACACAGCUACAUU |
| | | A | | |
| siRNA 3031 | 3031 | AUGUAGCUGUGUUCUGGA | 9385 | UUCCAGAACACAGCUACAU |
| | | A | | |
| siRNA 3032 | 3032 | UGUAGCUGUGUUCUGGAA | 9386 | GUUCCAGAACACAGCUACA |
| | | C | | |
| siRNA 3033 | 3033 | GUAGCUGUGUUCUGGAAC | 9387 | AGUUCCAGAACACAGCUAC |
| | | U | | |
| siRNA 3034 | 3034 | UAGCUGUGUUCUGGAACU | 9388 | CAGUUCCAGAACACAGCUA |
| | | G | | |
| siRNA 3035 | 3035 | AGCUGUGUUCUGGAACUG | 9389 | UCAGUUCCAGAACACAGCU |
| | | A | | |
| siRNA 3036 | 3036 | GCUGUGUUCUGGAACUGA | 9390 | UUCAGUUCCAGAACACAGC |
| | | A | | |
| siRNA 3037 | 3037 | CUGUGUUCUGGAACUGAA | 9391 | AUUCAGUUCCAGAACACAG |
| | | U | | |
| siRNA 3038 | 3038 | UGUGUUCUGGAACUGAAU | 9392 | GAUUCAGUUCCAGAACACA |
| | | C | | |
| siRNA 3039 | 3039 | GUGUUCUGGAACUGAAUC | 9393 | AGAUUCAGUUCCAGAACAC |
| | | U | | |
| siRNA 3040 | 3040 | UGUUCUGGAACUGAAUCU | 9394 | AAGAUUCAGUUCCAGAACA |
| | | U | | |
| siRNA 3041 | 3041 | GUUCUGGAACUGAAUCUU | 9395 | UAAGAUUCAGUUCCAGAAC |
| | | A | | |
| siRNA 3042 | 3042 | UUCUGGAACUGAAUCUUAC | 9396 | GUAAGAUUCAGUUCCAGA |
| | | | | A |
| siRNA 3043 | 3043 | UCUGGAACUGAAUCUUACA | 9397 | UGUAAGAUUCAGUUCCAG |
| | | | | A |
| siRNA 3044 | 3044 | CUGGAACUGAAUCUUACAU | 9398 | AUGUAAGAUUCAGUUCCA |
| | | | | G |
| siRNA 3045 | 3045 | UGGAACUGAAUCUUACAU | 9399 | CAUGUAAGAUUCAGUUCCA |
| | | G | | |
| siRNA 3046 | 3046 | GGAACUGAAUCUUACAUG | 9400 | CCAUGUAAGAUUCAGUUCC |
| | | G | | |
| siRNA 3047 | 3047 | GAACUGAAUCUUACAUGG | 9401 | ACCAUGUAAGAUUCAGUUC |
| | | U | | |
| siRNA 3048 | 3048 | AACUGAAUCUUACAUGGU | 9402 | UACCAUGUAAGAUUCAGU |
| | | A | | U |
| siRNA 3049 | 3049 | ACUGAAUCUUACAUGGUA | 9403 | AUACCAUGUAAGAUUCAG |
| | | U | | U |
| siRNA 3050 | 3050 | CUGAAUCUUACAUGGUAU | 9404 | UAUACCAUGUAAGAUUCA |
| | | A | | G |
| siRNA 3051 | 3051 | UGAAUCUUACAUGGUAUA | 9405 | GUAUACCAUGUAAGAUUC |
| | | C | | A |
| siRNA 3052 | 3052 | GAAUCUUACAUGGUAUAC | 9406 | AGUAUACCAUGUAAGAUU |
| | | U | | C |
| siRNA 3053 | 3053 | AAUCUUACAUGGUAUACU | 9407 | AAGUAUACCAUGUAAGAU |
| | | U | | U |
| siRNA 3054 | 3054 | AUCUUACAUGGUAUACUU | 9408 | UAAGUAUACCAUGUAAGA |
| | | A | | U |
| siRNA 3055 | 3055 | UCUUACAUGGUAUACUUA | 9409 | CUAAGUAUACCAUGUAAG |
| | | G | | A |
| siRNA 3056 | 3056 | CUUACAUGGUAUACUUAG | 9410 | ACUAAGUAUACCAUGUAA |
| | | U | | G |
| siRNA 3057 | 3057 | UUACAUGGUAUACUUAGU | 9411 | CACUAAGUAUACCAUGUAA |
| | | G | | |
| siRNA 3058 | 3058 | UACAUGGUAUACUUAGUG | 9412 | GCACUAAGUAUACCAUGUA |
| | | C | | |
| siRNA 3059 | 3059 | ACAUGGUAUACUUAGUGC | 9413 | AGCACUAAGUAUACCAUGU |
| | | U | | |
| siRNA 3060 | 3060 | CAUGGUAUACUUAGUGCU | 9414 | CAGCACUAAGUAUACCAUG |
| | | G | | |
| siRNA 3061 | 3061 | AUGGUAUACUUAGUGCUG | 9415 | GCAGCACUAAGUAUACCAU |
| | | C | | |
| siRNA 3062 | 3062 | UGGUAUACUUAGUGCUGC | 9416 | AGCAGCACUAAGUAUACCA |
| | | U | | |
| siRNA 3063 | 3063 | GGUAUACUUAGUGCUGCU | 9417 | CAGCAGCACUAAGUAUACC |
| | | G | | |
| siRNA 3064 | 3064 | GUAUACUUAGUGCUGCUG | 9418 | CCAGCAGCACUAAGUAUAC |
| | | G | | |
| siRNA 3065 | 3065 | UAUACUUAGUGCUGCUGG | 9419 | CCCAGCAGCACUAAGUAUA |
| | | G | | |
| siRNA 3066 | 3066 | AUACUUAGUGCUGCUGGG | 9420 | ACCCAGCAGCACUAAGUAU |
| | | U | | |
| siRNA 3067 | 3067 | UACUUAGUGCUGCUGGGU | 9421 | UACCCAGCAGCACUAAGUA |
| | | A | | |
| siRNA 3068 | 3068 | ACUUAGUGCUGCUGGGUA | 9422 | UUACCCAGCAGCACUAAGU |
| | | A | | |
| siRNA 3069 | 3069 | CUUAGUGCUGCUGGGUAA | 9423 | AUUACCCAGCAGCACUAAG |
| | | U | | |
| siRNA 3070 | 3070 | UUAGUGCUGCUGGGUAAU | 9424 | AAUUACCCAGCAGCACUAA |
| | | U | | |
| siRNA 3071 | 3071 | UAGUGCUGCUGGGUAAUU | 9425 | AAAUUACCCAGCAGCACUA |
| | | U | | |
| siRNA 3072 | 3072 | AGUGCUGCUGGGUAAUUU | 9426 | CAAAUUACCCAGCAGCACU |
| | | G | | |
| siRNA 3073 | 3073 | GUGCUGCUGGGUAAUUUG | 9427 | ACAAAUUACCCAGCAGCAC |
| | | U | | |
| siRNA 3074 | 3074 | UGCUGCUGGGUAAUUUGU | 9428 | AACAAAUUACCCAGCAGCA |
| | | U | | |
| siRNA 3075 | 3075 | GCUGCUGGGUAAUUUGUU | 9429 | CAACAAAUUACCCAGCAGC |
| | | G | | |
| siRNA 3076 | 3076 | CUGCUGGGUAAUUUGUUG | 9430 | CCAACAAAUUACCCAGCAG |
| | | G | | |
| siRNA 3077 | 3077 | UGCUGGGUAAUUUGUUGG | 9431 | ACCAACAAAUUACCCAGCA |
| | | U | | |
| siRNA 3078 | 3078 | GCUGGGUAAUUUGUUGGU | 9432 | UACCAACAAAUUACCCAGC |
| | | A | | |
| siRNA 3079 | 3079 | CUGGGUAAUUUGUUGGUA | 9433 | AUACCAACAAAUUACCCAG |
| | | U | | |
| siRNA 3080 | 3080 | UGGGUAAUUUGUUGGUAU | 9434 | UAUACCAACAAAUUACCCA |
| | | A | | |
| siRNA 3081 | 3081 | GGGUAAUUUGUUGGUAUA | 9435 | AUAUACCAACAAAUUACCC |
| | | U | | |
| siRNA 3082 | 3082 | GGUAAUUUGUUGGUAUAU | 9436 | AAUAUACCAACAAAUUACC |
| | | U | | |
| siRNA 3083 | 3083 | GUAAUUUGUUGGUAUAUU | 9437 | UAAUAUACCAACAAAUUAC |
| | | A | | |
| siRNA 3084 | 3084 | UAAUUUGUUGGUAUAUUA | 9438 | AUAAUAUACCAACAAAUU |
| | | U | | A |
| siRNA 3085 | 3085 | AAUUUGUUGGUAUAUUAU | 9439 | GAUAAUAUACCAACAAAU |
| | | C | | U |
| siRNA 3086 | 3086 | AUUUGUUGGUAUAUUAUC | 9440 | AGAUAAUAUACCAACAAA |
| | | U | | U |
| siRNA 3087 | 3087 | UUUGUUGGUAUAUUAUCU | 9441 | CAGAUAAUAUACCAACAAA |
| | | G | | |
| siRNA 3088 | 3088 | UUGUUGGUAUAUUAUCUG | 9442 | CCAGAUAAUAUACCAACAA |
| | | G | | |
| siRNA 3089 | 3089 | UGUUGGUAUAUUAUCUGG | 9443 | ACCAGAUAAUAUACCAACA |
| | | U | | |
| siRNA 3090 | 3090 | GUUGGUAUAUUAUCUGGU | 9444 | AACCAGAUAAUAUACCAAC |
| | | U | | |
| siRNA 3091 | 3091 | UUGGUAUAUUAUCUGGUU | 9445 | UAACCAGAUAAUAUACCAA |
| | | A | | |
| siRNA 3092 | 3092 | UGGUAUAUUAUCUGGUUA | 9446 | CUAACCAGAUAAUAUACCA |
| | | G | | |
| siRNA 3093 | 3093 | GGUAUAUUAUCUGGUUAG | 9447 | ACUAACCAGAUAAUAUACC |
| | | U | | |
| siRNA 3094 | 3094 | GUAUAUUAUCUGGUUAGU | 9448 | CACUAACCAGAUAAUAUAC |
| | | G | | |
| siRNA 3095 | 3095 | UAUAUUAUCUGGUUAGUG | 9449 | CCACUAACCAGAUAAUAUA |
| | | G | | |
| siRNA 3096 | 3096 | AUAUUAUCUGGUUAGUGG | 9450 | ACCACUAACCAGAUAAUAU |
| | | U | | |
| siRNA 3097 | 3097 | UAUUAUCUGGUUAGUGGU | 9451 | AACCACUAACCAGAUAAUA |
| | | U | | |
| siRNA 3098 | 3098 | AUUAUCUGGUUAGUGGUU | 9452 | UAACCACUAACCAGAUAAU |
| | | A | | |
| siRNA 3099 | 3099 | UUAUCUGGUUAGUGGUUA | 9453 | UUAACCACUAACCAGAUAA |
| | | A | | |
| siRNA 3100 | 3100 | UAUCUGGUUAGUGGUUAA | 9454 | AUUAACCACUAACCAGAUA |
| | | U | | |
| siRNA 3101 | 3101 | AUCUGGUUAGUGGUUAAU | 9455 | CAUUAACCACUAACCAGAU |
| | | G | | |
| siRNA 3102 | 3102 | UCUGGUUAGUGGUUAAUG | 9456 | GCAUUAACCACUAACCAGA |
| | | C | | |
| siRNA 3103 | 3103 | CUGGUUAGUGGUUAAUGC | 9457 | AGCAUUAACCACUAACCAG |
| | | U | | |
| siRNA 3104 | 3104 | UGGUUAGUGGUUAAUGCU | 9458 | AAGCAUUAACCACUAACCA |
| | | U | | |
| siRNA 3105 | 3105 | GGUUAGUGGUUAAUGCUU | 9459 | GAAGCAUUAACCACUAACC |
| | | C | | |
| siRNA 3106 | 3106 | GUUAGUGGUUAAUGCUUC | 9460 | GGAAGCAUUAACCACUAAC |
| | | C | | |
| siRNA 3107 | 3107 | UUAGUGGUUAAUGCUUCC | 9461 | AGGAAGCAUUAACCACUAA |
| | | U | | |
| siRNA 3108 | 3108 | UAGUGGUUAAUGCUUCCU | 9462 | AAGGAAGCAUUAACCACUA |
| | | U | | |
| siRNA 3109 | 3109 | AGUGGUUAAUGCUUCCUU | 9463 | AAAGGAAGCAUUAACCACU |
| | | U | | |
| siRNA 3110 | 3110 | GUGGUUAAUGCUUCCUUU | 9464 | UAAAGGAAGCAUUAACCAC |
| | | A | | |
| siRNA 3111 | 3111 | UGGUUAAUGCUUCCUUUA | 9465 | UUAAAGGAAGCAUUAACC |
| | | A | | A |
| siRNA 3112 | 3112 | GGUUAAUGCUUCCUUUAA | 9466 | UUUAAAGGAAGCAUUAAC |
| | | A | | C |
| siRNA 3113 | 3113 | GUUAAUGCUUCCUUUAAA | 9467 | UUUUAAAGGAAGCAUUAA |
| | | A | | C |
| siRNA 3114 | 3114 | UUAAUGCUUCCUUUAAAA | 9468 | UUUUUAAAGGAAGCAUUA |
| | | A | | A |
| siRNA 3115 | 3115 | UAAUGCUUCCUUUAAAAA | 9469 | AUUUUUAAAGGAAGCAUU |
| | | U | | A |
| siRNA 3116 | 3116 | AAUGCUUCCUUUAAAAAU | 9470 | UAUUUUUAAAGGAAGCAU |
| | | A | | U |
| siRNA 3117 | 3117 | AUGCUUCCUUUAAAAAUA | 9471 | UUAUUUUUAAAGGAAGCA |
| | | A | | U |
| siRNA 3118 | 3118 | UGCUUCCUUUAAAAAUAA | 9472 | AUUAUUUUUAAAGGAAGC |
| | | U | | A |
| siRNA 3119 | 3119 | GCUUCCUUUAAAAAUAAU | 9473 | AAUUAUUUUUAAAGGAAG |
| | | U | | C |
| siRNA 3120 | 3120 | CUUCCUUUAAAAAUAAUU | 9474 | CAAUUAUUUUUAAAGGAA |
| | | G | | G |
| siRNA 3121 | 3121 | UUCCUUUAAAAAUAAUUG | 9475 | UCAAUUAUUUUUAAAGGA |
| | | A | | A |
| siRNA 3122 | 3122 | UCCUUUAAAAAUAAUUGA | 9476 | CUCAAUUAUUUUUAAAGG |
| | | G | | A |
| siRNA 3123 | 3123 | CCUUUAAAAAUAAUUGAG | 9477 | ACUCAAUUAUUUUUAAAG |
| | | U | | G |
| siRNA 3124 | 3124 | CUUUAAAAAUAAUUGAGU | 9478 | GACUCAAUUAUUUUUAAA |
| | | C | | G |
| siRNA 3125 | 3125 | UUUAAAAAUAAUUGAGUC | 9479 | UGACUCAAUUAUUUUUAA |
| | | A | | A |
| siRNA 3126 | 3126 | UUAAAAAUAAUUGAGUCA | 9480 | AUGACUCAAUUAUUUUUA |
| | | U | | A |
| siRNA 3127 | 3127 | UAAAAAUAAUUGAGUCAU | 9481 | GAUGACUCAAUUAUUUUU |
| | | C | | A |
| siRNA 3128 | 3128 | AAAAAUAAUUGAGUCAUC | 9482 | GGAUGACUCAAUUAUUUU |
| | | C | | U |
| siRNA 3129 | 3129 | AAAAUAAUUGAGUCAUCC | 9483 | UGGAUGACUCAAUUAUUU |
| | | A | | U |
| siRNA 3130 | 3130 | AAAUAAUUGAGUCAUCCA | 9484 | AUGGAUGACUCAAUUAUU |
| | | U | | U |
| siRNA 3131 | 3131 | AAUAAUUGAGUCAUCCAU | 9485 | AAUGGAUGACUCAAUUAU |
| | | U | | U |
| siRNA 3132 | 3132 | AUAAUUGAGUCAUCCAUUC | 9486 | GAAUGGAUGACUCAAUUA |
| | | | | U |
| siRNA 3133 | 3133 | UAAUUGAGUCAUCCAUUCA | 9487 | UGAAUGGAUGACUCAAUU |
| | | | | A |
| siRNA 3134 | 3134 | AAUUGAGUCAUCCAUUCAC | 9488 | GUGAAUGGAUGACUCAAU |
| | | | | U |
| siRNA 3135 | 3135 | AUUGAGUCAUCCAUUCACU | 9489 | AGUGAAUGGAUGACUCAA |
| | | | | U |
| siRNA 3136 | 3136 | UUGAGUCAUCCAUUCACUC | 9490 | GAGUGAAUGGAUGACUCA |
| | | | | A |
| siRNA 3137 | 3137 | UGAGUCAUCCAUUCACUCU | 9491 | AGAGUGAAUGGAUGACUC |
| | | | | A |
| siRNA 3138 | 3138 | GAGUCAUCCAUUCACUCUU | 9492 | AAGAGUGAAUGGAUGACU |
| | | | | C |
| siRNA 3139 | 3139 | AGUCAUCCAUUCACUCUUU | 9493 | AAAGAGUGAAUGGAUGAC |
| | | | | U |
| siRNA 3140 | 3140 | GUCAUCCAUUCACUCUUUU | 9494 | AAAAGAGUGAAUGGAUGA |
| | | | | C |
| siRNA 3141 | 3141 | UCAUCCAUUCACUCUUUUU | 9495 | AAAAAGAGUGAAUGGAUG |
| | | | | A |
| siRNA 3142 | 3142 | CAUCCAUUCACUCUUUUUC | 9496 | GAAAAAGAGUGAAUGGAU |
| | | | | G |
| siRNA 3143 | 3143 | AUCCAUUCACUCUUUUUCA | 9497 | UGAAAAAGAGUGAAUGGA |
| | | | | U |
| siRNA 3144 | 3144 | UCCAUUCACUCUUUUUCAG | 9498 | CUGAAAAAGAGUGAAUGG |
| | | | | A |
| siRNA 3145 | 3145 | CCAUUCACUCUUUUUCAGU | 9499 | ACUGAAAAAGAGUGAAUG |
| | | | | G |
| siRNA 3146 | 3146 | CAUUCACUCUUUUUCAGUU | 9500 | AACUGAAAAAGAGUGAAU |
| | | | | G |
| siRNA 3147 | 3147 | AUUCACUCUUUUUCAGUUU | 9501 | AAACUGAAAAAGAGUGAA |
| | | | | U |
| siRNA 3148 | 3148 | UUCACUCUUUUUCAGUUUU | 9502 | AAAACUGAAAAAGAGUGA |
| | | | | A |
| siRNA 3149 | 3149 | UCACUCUUUUUCAGUUUUA | 9503 | UAAAACUGAAAAAGAGUG |
| | | | | A |
| siRNA 3150 | 3150 | CACUCUUUUUCAGUUUUAU | 9504 | AUAAAACUGAAAAAGAGU |
| | | | | G |
| siRNA 3151 | 3151 | ACUCUUUUUCAGUUUUAUC | 9505 | GAUAAAACUGAAAAAGAG |
| | | | | U |
| siRNA 3152 | 3152 | CUCUUUUUCAGUUUUAUCU | 9506 | AGAUAAAACUGAAAAAGA |
| | | | | G |
| siRNA 3153 | 3153 | UCUUUUUCAGUUUUAUCU | 9507 | CAGAUAAAACUGAAAAAG |
| | | G | | A |
| siRNA 3154 | 3154 | CUUUUUCAGUUUUAUCUG | 9508 | ACAGAUAAAACUGAAAAA |
| | | U | | G |
| siRNA 3155 | 3155 | UUUUUCAGUUUUAUCUGU | 9509 | GACAGAUAAAACUGAAAA |
| | | C | | A |
| siRNA 3156 | 3156 | UUUUCAGUUUUAUCUGUC | 9510 | UGACAGAUAAAACUGAAA |
| | | A | | A |
| siRNA 3157 | 3157 | UUUCAGUUUUAUCUGUCA | 9511 | UUGACAGAUAAAACUGAA |
| | | A | | A |
| siRNA 3158 | 3158 | UUCAGUUUUAUCUGUCAA | 9512 | AUUGACAGAUAAAACUGA |
| | | U | | A |
| siRNA 3159 | 3159 | UCAGUUUUAUCUGUCAAU | 9513 | UAUUGACAGAUAAAACUG |
| | | A | | A |
| siRNA 3160 | 3160 | CAGUUUUAUCUGUCAAUA | 9514 | CUAUUGACAGAUAAAACU |
| | | G | | G |
| siRNA 3161 | 3161 | AGUUUUAUCUGUCAAUAG | 9515 | ACUAUUGACAGAUAAAAC |
| | | U | | U |
| siRNA 3162 | 3162 | GUUUUAUCUGUCAAUAGU | 9516 | UACUAUUGACAGAUAAAA |
| | | A | | C |
| siRNA 3163 | 3163 | UUUUAUCUGUCAAUAGUA | 9517 | CUACUAUUGACAGAUAAA |
| | | G | | A |
| siRNA 3164 | 3164 | UUUAUCUGUCAAUAGUAG | 9518 | GCUACUAUUGACAGAUAA |
| | | C | | A |
| siRNA 3165 | 3165 | UUAUCUGUCAAUAGUAGC | 9519 | AGCUACUAUUGACAGAUA |
| | | U | | A |
| siRNA 3166 | 3166 | UAUCUGUCAAUAGUAGCU | 9520 | UAGCUACUAUUGACAGAU |
| | | A | | A |
| siRNA 3167 | 3167 | AUCUGUCAAUAGUAGCUAC | 9521 | GUAGCUACUAUUGACAGA |
| | | | | U |
| siRNA 3168 | 3168 | UCUGUCAAUAGUAGCUACA | 9522 | UGUAGCUACUAUUGACAG |
| | | | | A |
| siRNA 3169 | 3169 | CUGUCAAUAGUAGCUACAU | 9523 | AUGUAGCUACUAUUGACA |
| | | | | G |
| siRNA 3170 | 3170 | UGUCAAUAGUAGCUACAU | 9524 | AAUGUAGCUACUAUUGAC |
| | | U | | A |
| siRNA 3171 | 3171 | GUCAAUAGUAGCUACAUU | 9525 | AAAUGUAGCUACUAUUGA |
| | | U | | C |
| siRNA 3172 | 3172 | UCAAUAGUAGCUACAUUU | 9526 | AAAAUGUAGCUACUAUUG |
| | | U | | A |
| siRNA 3173 | 3173 | CAAUAGUAGCUACAUUUU | 9527 | AAAAAUGUAGCUACUAUU |
| | | U | | G |
| siRNA 3174 | 3174 | AAUAGUAGCUACAUUUUU | 9528 | UAAAAAUGUAGCUACUAU |
| | | A | | U |
| siRNA 3175 | 3175 | AUAGUAGCUACAUUUUUA | 9529 | UUAAAAAUGUAGCUACUA |
| | | A | | U |
| siRNA 3176 | 3176 | UAGUAGCUACAUUUUUAA | 9530 | AUUAAAAAUGUAGCUACU |
| | | U | | A |
| siRNA 3177 | 3177 | AGUAGCUACAUUUUUAAU | 9531 | CAUUAAAAAUGUAGCUAC |
| | | G | | U |
| siRNA 3178 | 3178 | GUAGCUACAUUUUUAAUG | 9532 | CCAUUAAAAAUGUAGCUAC |
| | | G | | |
| siRNA 3179 | 3179 | UAGCUACAUUUUUAAUGG | 9533 | CCCAUUAAAAAUGUAGCUA |
| | | G | | |
| siRNA 3180 | 3180 | AGCUACAUUUUUAAUGGG | 9534 | UCCCAUUAAAAAUGUAGCU |
| | | A | | |
| siRNA 3181 | 3181 | GCUACAUUUUUAAUGGGA | 9535 | CUCCCAUUAAAAAUGUAGC |
| | | G | | |
| siRNA 3182 | 3182 | CUACAUUUUUAAUGGGAG | 9536 | GCUCCCAUUAAAAAUGUAG |
| | | C | | |
| siRNA 3183 | 3183 | UACAUUUUUAAUGGGAGC | 9537 | UGCUCCCAUUAAAAAUGUA |
| | | A | | |
| siRNA 3184 | 3184 | ACAUUUUUAAUGGGAGCA | 9538 | GUGCUCCCAUUAAAAAUGU |
| | | C | | |
| siRNA 3185 | 3185 | CAUUUUUAAUGGGAGCACC | 9539 | GGUGCUCCCAUUAAAAAUG |
| siRNA 3186 | 3186 | AUUUUUAAUGGGAGCACC | 9540 | AGGUGCUCCCAUUAAAAAU |
| | | U | | |
| siRNA 3187 | 3187 | UUUUUAAUGGGAGCACCU | 9541 | AAGGUGCUCCCAUUAAAAA |
| | | U | | |
| siRNA 3188 | 3188 | UUUUAAUGGGAGCACCUU | 9542 | AAAGGUGCUCCCAUUAAAA |
| | | U | | |
| siRNA 3189 | 3189 | UUUAAUGGGAGCACCUUU | 9543 | AAAAGGUGCUCCCAUUAAA |
| | | U | | |
| siRNA 3190 | 3190 | UUAAUGGGAGCACCUUUU | 9544 | UAAAAGGUGCUCCCAUUAA |
| | | A | | |
| siRNA 3191 | 3191 | UAAUGGGAGCACCUUUUA | 9545 | AUAAAAGGUGCUCCCAUUA |
| | | U | | |
| siRNA 3192 | 3192 | AAUGGGAGCACCUUUUAUC | 9546 | GAUAAAAGGUGCUCCCAUU |
| siRNA 3193 | 3193 | AUGGGAGCACCUUUUAUCC | 9547 | GGAUAAAAGGUGCUCCCAU |
| siRNA 3194 | 3194 | UGGGAGCACCUUUUAUCCC | 9548 | GGGAUAAAAGGUGCUCCCA |
| siRNA 3195 | 3195 | GGGAGCACCUUUUAUCCCA | 9549 | UGGGAUAAAAGGUGCUCCC |
| siRNA 3196 | 3196 | GGAGCACCUUUUAUCCCAA | 9550 | UUGGGAUAAAAGGUGCUC |
| | | | | C |
| siRNA 3197 | 3197 | GAGCACCUUUUAUCCCAAA | 9551 | UUUGGGAUAAAAGGUGCU |
| | | | | C |
| siRNA 3198 | 3198 | AGCACCUUUUAUCCCAAAG | 9552 | CUUUGGGAUAAAAGGUGC |
| | | | | U |
| siRNA 3199 | 3199 | GCACCUUUUAUCCCAAAGU | 9553 | ACUUUGGGAUAAAAGGUG |
| | | | | C |
| siRNA 3200 | 3200 | CACCUUUUAUCCCAAAGUG | 9554 | CACUUUGGGAUAAAAGGU |
| | | | | G |
| siRNA 3201 | 3201 | ACCUUUUAUCCCAAAGUGC | 9555 | GCACUUUGGGAUAAAAGG |
| | | | | U |
| siRNA 3202 | 3202 | CCUUUUAUCCCAAAGUGCU | 9556 | AGCACUUUGGGAUAAAAG |
| | | | | G |
| siRNA 3203 | 3203 | CUUUUAUCCCAAAGUGCUU | 9557 | AAGCACUUUGGGAUAAAA |
| | | | | G |
| siRNA 3204 | 3204 | UUUUAUCCCAAAGUGCUUU | 9558 | AAAGCACUUUGGGAUAAA |
| | | | | A |
| siRNA 3205 | 3205 | UUUAUCCCAAAGUGCUUUA | 9559 | UAAAGCACUUUGGGAUAA |
| | | | | A |
| siRNA 3206 | 3206 | UUAUCCCAAAGUGCUUUAU | 9560 | AUAAAGCACUUUGGGAUA |
| | | | | A |
| siRNA 3207 | 3207 | UAUCCCAAAGUGCUUUAUA | 9561 | UAUAAAGCACUUUGGGAU |
| | | | | A |
| siRNA 3208 | 3208 | AUCCCAAAGUGCUUUAUAA | 9562 | UUAUAAAGCACUUUGGGA |
| | | | | U |
| siRNA 3209 | 3209 | UCCCAAAGUGCUUUAUAAA | 9563 | UUUAUAAAGCACUUUGGG |
| | | | | A |
| siRNA 3210 | 3210 | CCCAAAGUGCUUUAUAAAU | 9564 | AUUUAUAAAGCACUUUGG |
| | | | | G |
| siRNA 3211 | 3211 | CCAAAGUGCUUUAUAAAU | 9565 | AAUUUAUAAAGCACUUUG |
| | | U | | G |
| siRNA 3212 | 3212 | CAAAGUGCUUUAUAAAUU | 9566 | CAAUUUAUAAAGCACUUU |
| | | G | | G |
| siRNA 3213 | 3213 | AAAGUGCUUUAUAAAUUG | 9567 | UCAAUUUAUAAAGCACUU |
| | | A | | U |
| siRNA 3214 | 3214 | AAGUGCUUUAUAAAUUGA | 9568 | CUCAAUUUAUAAAGCACUU |
| | | G | | |
| siRNA 3215 | 3215 | AGUGCUUUAUAAAUUGAG | 9569 | ACUCAAUUUAUAAAGCACU |
| | | U | | |
| siRNA 3216 | 3216 | GUGCUUUAUAAAUUGAGU | 9570 | CACUCAAUUUAUAAAGCAC |
| | | G | | |
| siRNA 3217 | 3217 | UGCUUUAUAAAUUGAGUG | 9571 | CCACUCAAUUUAUAAAGCA |
| | | G | | |
| siRNA 3218 | 3218 | GCUUUAUAAAUUGAGUGG | 9572 | UCCACUCAAUUUAUAAAGC |
| | | A | | |
| siRNA 3219 | 3219 | CUUUAUAAAUUGAGUGGA | 9573 | GUCCACUCAAUUUAUAAAG |
| | | C | | |
| siRNA 3220 | 3220 | UUUAUAAAUUGAGUGGAC | 9574 | AGUCCACUCAAUUUAUAAA |
| | | U | | |
| siRNA 3221 | 3221 | UUAUAAAUUGAGUGGACU | 9575 | CAGUCCACUCAAUUUAUAA |
| | | G | | |
| siRNA 3222 | 3222 | UAUAAAUUGAGUGGACUG | 9576 | UCAGUCCACUCAAUUUAUA |
| | | A | | |
| siRNA 3223 | 3223 | AUAAAUUGAGUGGACUGA | 9577 | AUCAGUCCACUCAAUUUAU |
| | | U | | |
| siRNA 3224 | 3224 | UAAAUUGAGUGGACUGAU | 9578 | UAUCAGUCCACUCAAUUUA |
| | | A | | |
| siRNA 3225 | 3225 | AAAUUGAGUGGACUGAUA | 9579 | AUAUCAGUCCACUCAAUUU |
| | | U | | |
| siRNA 3226 | 3226 | AAUUGAGUGGACUGAUAU | 9580 | UAUAUCAGUCCACUCAAUU |
| | | A | | |
| siRNA 3227 | 3227 | AUUGAGUGGACUGAUAUA | 9581 | AUAUAUCAGUCCACUCAAU |
| | | U | | |
| siRNA 3228 | 3228 | UUGAGUGGACUGAUAUAU | 9582 | UAUAUAUCAGUCCACUCAA |
| | | A | | |
| siRNA 3229 | 3229 | UGAGUGGACUGAUAUAUA | 9583 | AUAUAUAUCAGUCCACUCA |
| | | U | | |
| siRNA 3230 | 3230 | GAGUGGACUGAUAUAUAU | 9584 | GAUAUAUAUCAGUCCACUC |
| | | C | | |
| siRNA 3231 | 3231 | AGUGGACUGAUAUAUAUC | 9585 | UGAUAUAUAUCAGUCCACU |
| | | A | | |
| siRNA 3232 | 3232 | GUGGACUGAUAUAUAUCA | 9586 | GUGAUAUAUAUCAGUCCAC |
| | | C | | |
| siRNA 3233 | 3233 | UGGACUGAUAUAUAUCAC | 9587 | UGUGAUAUAUAUCAGUCC |
| | | A | | A |
| siRNA 3234 | 3234 | GGACUGAUAUAUAUCACAC | 9588 | GUGUGAUAUAUAUCAGUC |
| | | | | C |
| siRNA 3235 | 3235 | GACUGAUAUAUAUCACACC | 9589 | GGUGUGAUAUAUAUCAGU |
| | | | | C |
| siRNA 3236 | 3236 | ACUGAUAUAUAUCACACCC | 9590 | GGGUGUGAUAUAUAUCAG |
| | | | | U |
| siRNA 3237 | 3237 | CUGAUAUAUAUCACACCCA | 9591 | UGGGUGUGAUAUAUAUCA |
| | | | | G |
| siRNA 3238 | 3238 | UGAUAUAUAUCACACCCAG | 9592 | CUGGGUGUGAUAUAUAUC |
| | | | | A |
| siRNA 3239 | 3239 | GAUAUAUAUCACACCCAGG | 9593 | CCUGGGUGUGAUAUAUAU |
| | | | | C |
| siRNA 3240 | 3240 | AUAUAUAUCACACCCAGGU | 9594 | ACCUGGGUGUGAUAUAUA |
| | | | | U |
| siRNA 3241 | 3241 | UAUAUAUCACACCCAGGUA | 9595 | UACCUGGGUGUGAUAUAU |
| | | | | A |
| siRNA 3242 | 3242 | AUAUAUCACACCCAGGUAU | 9596 | AUACCUGGGUGUGAUAUA |
| | | | | U |
| siRNA 3243 | 3243 | UAUAUCACACCCAGGUAUC | 9597 | GAUACCUGGGUGUGAUAU |
| | | | | A |
| siRNA 3244 | 3244 | AUAUCACACCCAGGUAUCA | 9598 | UGAUACCUGGGUGUGAUA |
| | | | | U |
| siRNA 3245 | 3245 | UAUCACACCCAGGUAUCAC | 9599 | GUGAUACCUGGGUGUGAU |
| | | | | A |
| siRNA 3246 | 3246 | AUCACACCCAGGUAUCACU | 9600 | AGUGAUACCUGGGUGUGA |
| | | | | U |
| siRNA 3247 | 3247 | UCACACCCAGGUAUCACUG | 9601 | CAGUGAUACCUGGGUGUG |
| | | | | A |
| siRNA 3248 | 3248 | CACACCCAGGUAUCACUGU | 9602 | ACAGUGAUACCUGGGUGU |
| | | | | G |
| siRNA 3249 | 3249 | ACACCCAGGUAUCACUGUG | 9603 | CACAGUGAUACCUGGGUGU |
| siRNA 3250 | 3250 | CACCCAGGUAUCACUGUGC | 9604 | GCACAGUGAUACCUGGGUG |
| siRNA 3251 | 3251 | ACCCAGGUAUCACUGUGCU | 9605 | AGCACAGUGAUACCUGGGU |
| siRNA 3252 | 3252 | CCCAGGUAUCACUGUGCUG | 9606 | CAGCACAGUGAUACCUGGG |
| siRNA 3253 | 3253 | CCAGGUAUCACUGUGCUGU | 9607 | ACAGCACAGUGAUACCUGG |
| siRNA 3254 | 3254 | CAGGUAUCACUGUGCUGUC | 9608 | GACAGCACAGUGAUACCUG |
| siRNA 3255 | 3255 | AGGUAUCACUGUGCUGUCC | 9609 | GGACAGCACAGUGAUACCU |
| siRNA 3256 | 3256 | GGUAUCACUGUGCUGUCCU | 9610 | AGGACAGCACAGUGAUACC |
| siRNA 3257 | 3257 | GUAUCACUGUGCUGUCCUU | 9611 | AAGGACAGCACAGUGAUAC |
| siRNA 3258 | 3258 | UAUCACUGUGCUGUCCUUU | 9612 | AAAGGACAGCACAGUGAU |
| | | | | A |
| siRNA 3259 | 3259 | AUCACUGUGCUGUCCUUUG | 9613 | CAAAGGACAGCACAGUGAU |
| siRNA 3260 | 3260 | UCACUGUGCUGUCCUUUGC | 9614 | GCAAAGGACAGCACAGUGA |
| siRNA 3261 | 3261 | CACUGUGCUGUCCUUUGCU | 9615 | AGCAAAGGACAGCACAGUG |
| siRNA 3262 | 3262 | ACUGUGCUGUCCUUUGCUG | 9616 | CAGCAAAGGACAGCACAGU |
| siRNA 3263 | 3263 | CUGUGCUGUCCUUUGCUGU | 9617 | ACAGCAAAGGACAGCACAG |
| siRNA 3264 | 3264 | UGUGCUGUCCUUUGCUGUC | 9618 | GACAGCAAAGGACAGCACA |
| siRNA 3265 | 3265 | GUGCUGUCCUUUGCUGUCA | 9619 | UGACAGCAAAGGACAGCAC |
| siRNA 3266 | 3266 | UGCUGUCCUUUGCUGUCAG | 9620 | CUGACAGCAAAGGACAGCA |
| siRNA 3267 | 3267 | GCUGUCCUUUGCUGUCAGA | 9621 | UCUGACAGCAAAGGACAGC |
| siRNA 3268 | 3268 | CUGUCCUUUGCUGUCAGAU | 9622 | AUCUGACAGCAAAGGACAG |
| siRNA 3269 | 3269 | UGUCCUUUGCUGUCAGAUU | 9623 | AAUCUGACAGCAAAGGACA |
| siRNA 3270 | 3270 | GUCCUUUGCUGUCAGAUUU | 9624 | AAAUCUGACAGCAAAGGAC |
| siRNA 3271 | 3271 | UCCUUUGCUGUCAGAUUUA | 9625 | UAAAUCUGACAGCAAAGG |
| | | | | A |
| siRNA 3272 | 3272 | CCUUUGCUGUCAGAUUUAG | 9626 | CUAAAUCUGACAGCAAAGG |
| siRNA 3273 | 3273 | CUUUGCUGUCAGAUUUAG | 9627 | UCUAAAUCUGACAGCAAAG |
| | | A | | |
| siRNA 3274 | 3274 | UUUGCUGUCAGAUUUAGA | 9628 | UUCUAAAUCUGACAGCAAA |
| | | A | | |
| siRNA 3275 | 3275 | UUGCUGUCAGAUUUAGAA | 9629 | UUUCUAAAUCUGACAGCAA |
| | | A | | |
| siRNA 3276 | 3276 | UGCUGUCAGAUUUAGAAA | 9630 | AUUUCUAAAUCUGACAGCA |
| | | U | | |
| siRNA 3277 | 3277 | GCUGUCAGAUUUAGAAAU | 9631 | CAUUUCUAAAUCUGACAGC |
| | | G | | |
| siRNA 3278 | 3278 | CUGUCAGAUUUAGAAAUG | 9632 | ACAUUUCUAAAUCUGACAG |
| | | U | | |
| siRNA 3279 | 3279 | UGUCAGAUUUAGAAAUGU | 9633 | AACAUUUCUAAAUCUGACA |
| | | U | | |
| siRNA 3280 | 3280 | GUCAGAUUUAGAAAUGUU | 9634 | AAACAUUUCUAAAUCUGAC |
| | | U | | |
| siRNA 3281 | 3281 | UCAGAUUUAGAAAUGUUU | 9635 | AAAACAUUUCUAAAUCUG |
| | | U | | A |
| siRNA 3282 | 3282 | CAGAUUUAGAAAUGUUUU | 9636 | AAAAACAUUUCUAAAUCU |
| | | U | | G |
| siRNA 3283 | 3283 | AGAUUUAGAAAUGUUUUU | 9637 | UAAAAACAUUUCUAAAUC |
| | | A | | U |
| siRNA 3284 | 3284 | GAUUUAGAAAUGUUUUUA | 9638 | UUAAAAACAUUUCUAAAU |
| | | A | | C |
| siRNA 3285 | 3285 | AUUUAGAAAUGUUUUUAA | 9639 | CUUAAAAACAUUUCUAAA |
| | | G | | U |
| siRNA 3286 | 3286 | UUUAGAAAUGUUUUUAAG | 9640 | UCUUAAAAACAUUUCUAA |
| | | A | | A |
| siRNA 3287 | 3287 | UUAGAAAUGUUUUUAAGA | 9641 | CUCUUAAAAACAUUUCUAA |
| | | G | | |
| siRNA 3288 | 3288 | UAGAAAUGUUUUUAAGAG | 9642 | GCUCUUAAAAACAUUUCUA |
| | | C | | |
| siRNA 3289 | 3289 | AGAAAUGUUUUUAAGAGC | 9643 | AGCUCUUAAAAACAUUUCU |
| | | U | | |
| siRNA 3290 | 3290 | GAAAUGUUUUUAAGAGCU | 9644 | UAGCUCUUAAAAACAUUUC |
| | | A | | |
| siRNA 3291 | 3291 | AAAUGUUUUUAAGAGCUA | 9645 | AUAGCUCUUAAAAACAUU |
| | | U | | U |
| siRNA 3292 | 3292 | AAUGUUUUUAAGAGCUAU | 9646 | CAUAGCUCUUAAAAACAUU |
| | | G | | |
| siRNA 3293 | 3293 | AUGUUUUUAAGAGCUAUG | 9647 | ACAUAGCUCUUAAAAACAU |
| | | U | | |
| siRNA 3294 | 3294 | UGUUUUUAAGAGCUAUGU | 9648 | CACAUAGCUCUUAAAAACA |
| | | G | | |
| siRNA 3295 | 3295 | GUUUUUAAGAGCUAUGUG | 9649 | UCACAUAGCUCUUAAAAAC |
| | | A | | |
| siRNA 3296 | 3296 | UUUUUAAGAGCUAUGUGA | 9650 | UUCACAUAGCUCUUAAAAA |
| | | A | | |
| siRNA 3297 | 3297 | UUUUAAGAGCUAUGUGAA | 9651 | UUUCACAUAGCUCUUAAAA |
| | | A | | |
| siRNA 3298 | 3298 | UUUAAGAGCUAUGUGAAA | 9652 | UUUUCACAUAGCUCUUAAA |
| | | A | | |
| siRNA 3299 | 3299 | UUAAGAGCUAUGUGAAAA | 9653 | GUUUUCACAUAGCUCUUAA |
| | | C | | |
| siRNA 3300 | 3300 | UAAGAGCUAUGUGAAAAC | 9654 | UGUUUUCACAUAGCUCUUA |
| | | A | | |
| siRNA 3301 | 3301 | AAGAGCUAUGUGAAAACA | 9655 | CUGUUUUCACAUAGCUCUU |
| | | G | | |
| siRNA 3302 | 3302 | AGAGCUAUGUGAAAACAG | 9656 | UCUGUUUUCACAUAGCUCU |
| | | A | | |
| siRNA 3303 | 3303 | GAGCUAUGUGAAAACAGA | 9657 | GUCUGUUUUCACAUAGCUC |
| | | C | | |
| siRNA 3304 | 3304 | AGCUAUGUGAAAACAGAC | 9658 | UGUCUGUUUUCACAUAGCU |
| | | A | | |
| siRNA 3305 | 3305 | GCUAUGUGAAAACAGACA | 9659 | UUGUCUGUUUUCACAUAGC |
| | | A | | |
| siRNA 3306 | 3306 | CUAUGUGAAAACAGACAA | 9660 | AUUGUCUGUUUUCACAUA |
| | | U | | G |
| siRNA 3307 | 3307 | UAUGUGAAAACAGACAAU | 9661 | UAUUGUCUGUUUUCACAU |
| | | A | | A |
| siRNA 3308 | 3308 | AUGUGAAAACAGACAAUA | 9662 | AUAUUGUCUGUUUUCACA |
| | | U | | U |
| siRNA 3309 | 3309 | UGUGAAAACAGACAAUAU | 9663 | AAUAUUGUCUGUUUUCAC |
| | | U | | A |
| siRNA 3310 | 3310 | GUGAAAACAGACAAUAUU | 9664 | UAAUAUUGUCUGUUUUCA |
| | | A | | C |
| siRNA 3311 | 3311 | UGAAAACAGACAAUAUUA | 9665 | CUAAUAUUGUCUGUUUUC |
| | | G | | A |
| siRNA 3312 | 3312 | GAAAACAGACAAUAUUAG | 9666 | ACUAAUAUUGUCUGUUUU |
| | | U | | C |
| siRNA 3313 | 3313 | AAAACAGACAAUAUUAGU | 9667 | AACUAAUAUUGUCUGUUU |
| | | U | | U |
| siRNA 3314 | 3314 | AAACAGACAAUAUUAGUU | 9668 | AAACUAAUAUUGUCUGUU |
| | | U | | U |
| siRNA 3315 | 3315 | AACAGACAAUAUUAGUUU | 9669 | UAAACUAAUAUUGUCUGU |
| | | A | | U |
| siRNA 3316 | 3316 | ACAGACAAUAUUAGUUUA | 9670 | CUAAACUAAUAUUGUCUG |
| | | G | | U |
| siRNA 3317 | 3317 | CAGACAAUAUUAGUUUAG | 9671 | CCUAAACUAAUAUUGUCUG |
| | | G | | |
| siRNA 3318 | 3318 | AGACAAUAUUAGUUUAGG | 9672 | ACCUAAACUAAUAUUGUCU |
| | | U | | |
| siRNA 3319 | 3319 | GACAAUAUUAGUUUAGGU | 9673 | GACCUAAACUAAUAUUGUC |
| | | C | | |
| siRNA 3320 | 3320 | ACAAUAUUAGUUUAGGUC | 9674 | CGACCUAAACUAAUAUUGU |
| | | G | | |
| siRNA 3321 | 3321 | CAAUAUUAGUUUAGGUCG | 9675 | CCGACCUAAACUAAUAUUG |
| | | G | | |
| siRNA 3322 | 3322 | AAUAUUAGUUUAGGUCGG | 9676 | CCCGACCUAAACUAAUAUU |
| | | G | | |
| siRNA 3323 | 3323 | AUAUUAGUUUAGGUCGGG | 9677 | UCCCGACCUAAACUAAUAU |
| | | A | | |
| siRNA 3324 | 3324 | UAUUAGUUUAGGUCGGGA | 9678 | UUCCCGACCUAAACUAAUA |
| | | A | | |
| siRNA 3325 | 3325 | AUUAGUUUAGGUCGGGAA | 9679 | GUUCCCGACCUAAACUAAU |
| | | C | | |
| siRNA 3326 | 3326 | UUAGUUUAGGUCGGGAAC | 9680 | AGUUCCCGACCUAAACUAA |
| | | U | | |
| siRNA 3327 | 3327 | UAGUUUAGGUCGGGAACU | 9681 | CAGUUCCCGACCUAAACUA |
| | | G | | |
| siRNA 3328 | 3328 | AGUUUAGGUCGGGAACUG | 9682 | UCAGUUCCCGACCUAAACU |
| | | A | | |
| siRNA 3329 | 3329 | GUUUAGGUCGGGAACUGA | 9683 | CUCAGUUCCCGACCUAAAC |
| | | G | | |
| siRNA 3330 | 3330 | UUUAGGUCGGGAACUGAG | 9684 | UCUCAGUUCCCGACCUAAA |
| | | A | | |
| siRNA 3331 | 3331 | UUAGGUCGGGAACUGAGA | 9685 | AUCUCAGUUCCCGACCUAA |
| | | U | | |
| siRNA 3332 | 3332 | UAGGUCGGGAACUGAGAU | 9686 | UAUCUCAGUUCCCGACCUA |
| | | A | | |
| siRNA 3333 | 3333 | AGGUCGGGAACUGAGAUA | 9687 | AUAUCUCAGUUCCCGACCU |
| | | U | | |
| siRNA 3334 | 3334 | GGUCGGGAACUGAGAUAU | 9688 | AAUAUCUCAGUUCCCGACC |
| | | U | | |
| siRNA 3335 | 3335 | GUCGGGAACUGAGAUAUU | 9689 | CAAUAUCUCAGUUCCCGAC |
| | | G | | |
| siRNA 3336 | 3336 | UCGGGAACUGAGAUAUUG | 9690 | ACAAUAUCUCAGUUCCCGA |
| | | U | | |
| siRNA 3337 | 3337 | CGGGAACUGAGAUAUUGU | 9691 | UACAAUAUCUCAGUUCCCG |
| | | A | | |
| siRNA 3338 | 3338 | GGGAACUGAGAUAUUGUA | 9692 | UUACAAUAUCUCAGUUCCC |
| | | A | | |
| siRNA 3339 | 3339 | GGAACUGAGAUAUUGUAA | 9693 | AUUACAAUAUCUCAGUUCC |
| | | U | | |
| siRNA 3340 | 3340 | GAACUGAGAUAUUGUAAU | 9694 | GAUUACAAUAUCUCAGUUC |
| | | C | | |
| siRNA 3341 | 3341 | AACUGAGAUAUUGUAAUC | 9695 | UGAUUACAAUAUCUCAGU |
| | | A | | U |
| siRNA 3342 | 3342 | ACUGAGAUAUUGUAAUCA | 9696 | UUGAUUACAAUAUCUCAG |
| | | A | | U |
| siRNA 3343 | 3343 | CUGAGAUAUUGUAAUCAA | 9697 | UUUGAUUACAAUAUCUCA |
| | | A | | G |
| siRNA 3344 | 3344 | UGAGAUAUUGUAAUCAAA | 9698 | AUUUGAUUACAAUAUCUC |
| | | U | | A |
| siRNA 3345 | 3345 | GAGAUAUUGUAAUCAAAU | 9699 | UAUUUGAUUACAAUAUCU |
| | | A | | C |
| siRNA 3346 | 3346 | AGAUAUUGUAAUCAAAUA | 9700 | CUAUUUGAUUACAAUAUC |
| | | G | | U |
| siRNA 3347 | 3347 | GAUAUUGUAAUCAAAUAG | 9701 | ACUAUUUGAUUACAAUAU |
| | | U | | C |
| siRNA 3348 | 3348 | AUAUUGUAAUCAAAUAGU | 9702 | AACUAUUUGAUUACAAUA |
| | | U | | U |
| siRNA 3349 | 3349 | UAUUGUAAUCAAAUAGUU | 9703 | UAACUAUUUGAUUACAAU |
| | | A | | A |
| siRNA 3350 | 3350 | AUUGUAAUCAAAUAGUUA | 9704 | UUAACUAUUUGAUUACAA |
| | | A | | U |
| siRNA 3351 | 3351 | UUGUAAUCAAAUAGUUAA | 9705 | GUUAACUAUUUGAUUACA |
| | | C | | A |
| siRNA 3352 | 3352 | UGUAAUCAAAUAGUUAAC | 9706 | UGUUAACUAUUUGAUUAC |
| | | A | | A |
| siRNA 3353 | 3353 | GUAAUCAAAUAGUUAACA | 9707 | AUGUUAACUAUUUGAUUA |
| | | U | | C |
| siRNA 3354 | 3354 | UAAUCAAAUAGUUAACAU | 9708 | GAUGUUAACUAUUUGAUU |
| | | C | | A |
| siRNA 3355 | 3355 | AAUCAAAUAGUUAACAUC | 9709 | UGAUGUUAACUAUUUGAU |
| | | A | | U |
| siRNA 3356 | 3356 | AUCAAAUAGUUAACAUCA | 9710 | CUGAUGUUAACUAUUUGA |
| | | G | | U |
| siRNA 3357 | 3357 | UCAAAUAGUUAACAUCAG | 9711 | CCUGAUGUUAACUAUUUG |
| | | G | | A |
| siRNA 3358 | 3358 | CAAAUAGUUAACAUCAGG | 9712 | UCCUGAUGUUAACUAUUU |
| | | A | | G |
| siRNA 3359 | 3359 | AAAUAGUUAACAUCAGGA | 9713 | UUCCUGAUGUUAACUAUU |
| | | A | | U |
| siRNA 3360 | 3360 | AAUAGUUAACAUCAGGAA | 9714 | CUUCCUGAUGUUAACUAUU |
| | | G | | |
| siRNA 3361 | 3361 | AUAGUUAACAUCAGGAAG | 9715 | ACUUCCUGAUGUUAACUAU |
| | | U | | |
| siRNA 3362 | 3362 | UAGUUAACAUCAGGAAGU | 9716 | AACUUCCUGAUGUUAACUA |
| | | U | | |
| siRNA 3363 | 3363 | AGUUAACAUCAGGAAGUU | 9717 | UAACUUCCUGAUGUUAACU |
| | | A | | |
| siRNA 3364 | 3364 | GUUAACAUCAGGAAGUUA | 9718 | UUAACUUCCUGAUGUUAAC |
| | | A | | |
| siRNA 3365 | 3365 | UUAACAUCAGGAAGUUAA | 9719 | AUUAACUUCCUGAUGUUA |
| | | U | | A |
| siRNA 3366 | 3366 | UAACAUCAGGAAGUUAAU | 9720 | AAUUAACUUCCUGAUGUU |
| | | U | | A |
| siRNA 3367 | 3367 | AACAUCAGGAAGUUAAUU | 9721 | AAAUUAACUUCCUGAUGU |
| | | U | | U |
| siRNA 3368 | 3368 | ACAUCAGGAAGUUAAUUU | 9722 | CAAAUUAACUUCCUGAUGU |
| | | G | | |
| siRNA 3369 | 3369 | CAUCAGGAAGUUAAUUUG | 9723 | CCAAAUUAACUUCCUGAUG |
| | | G | | |
| siRNA 3370 | 3370 | AUCAGGAAGUUAAUUUGG | 9724 | GCCAAAUUAACUUCCUGAU |
| | | C | | |
| siRNA 3371 | 3371 | UCAGGAAGUUAAUUUGGC | 9725 | AGCCAAAUUAACUUCCUGA |
| | | U | | |
| siRNA 3372 | 3372 | CAGGAAGUUAAUUUGGCU | 9726 | CAGCCAAAUUAACUUCCUG |
| | | G | | |
| siRNA 3373 | 3373 | AGGAAGUUAAUUUGGCUG | 9727 | CCAGCCAAAUUAACUUCCU |
| | | G | | |
| siRNA 3374 | 3374 | GGAAGUUAAUUUGGCUGG | 9728 | GCCAGCCAAAUUAACUUCC |
| | | C | | |
| siRNA 3375 | 3375 | GAAGUUAAUUUGGCUGGC | 9729 | UGCCAGCCAAAUUAACUUC |
| | | A | | |
| siRNA 3376 | 3376 | AAGUUAAUUUGGCUGGCA | 9730 | UUGCCAGCCAAAUUAACUU |
| | | A | | |
| siRNA 3377 | 3377 | AGUUAAUUUGGCUGGCAA | 9731 | UUUGCCAGCCAAAUUAACU |
| | | A | | |
| siRNA 3378 | 3378 | GUUAAUUUGGCUGGCAAA | 9732 | UUUUGCCAGCCAAAUUAAC |
| | | A | | |
| siRNA 3379 | 3379 | UUAAUUUGGCUGGCAAAA | 9733 | AUUUUGCCAGCCAAAUUAA |
| | | U | | |
| siRNA 3380 | 3380 | UAAUUUGGCUGGCAAAAU | 9734 | AAUUUUGCCAGCCAAAUUA |
| | | U | | |
| siRNA 3381 | 3381 | AAUUUGGCUGGCAAAAUU | 9735 | GAAUUUUGCCAGCCAAAUU |
| | | C | | |
| siRNA 3382 | 3382 | AUUUGGCUGGCAAAAUUC | 9736 | AGAAUUUUGCCAGCCAAAU |
| | | U | | |
| siRNA 3383 | 3383 | UUUGGCUGGCAAAAUUCU | 9737 | UAGAAUUUUGCCAGCCAAA |
| | | A | | |
| siRNA 3384 | 3384 | UUGGCUGGCAAAAUUCUA | 9738 | CUAGAAUUUUGCCAGCCAA |
| | | G | | |
| siRNA 3385 | 3385 | UGGCUGGCAAAAUUCUAG | 9739 | CCUAGAAUUUUGCCAGCCA |
| | | G | | |
| siRNA 3386 | 3386 | GGCUGGCAAAAUUCUAGG | 9740 | CCCUAGAAUUUUGCCAGCC |
| | | G | | |
| siRNA 3387 | 3387 | GCUGGCAAAAUUCUAGGG | 9741 | UCCCUAGAAUUUUGCCAGC |
| | | A | | |
| siRNA 3388 | 3388 | CUGGCAAAAUUCUAGGGA | 9742 | UUCCCUAGAAUUUUGCCAG |
| | | A | | |
| siRNA 3389 | 3389 | UGGCAAAAUUCUAGGGAA | 9743 | UUUCCCUAGAAUUUUGCCA |
| | | A | | |
| siRNA 3390 | 3390 | GGCAAAAUUCUAGGGAAA | 9744 | GUUUCCCUAGAAUUUUGCC |
| | | C | | |
| siRNA 3391 | 3391 | GCAAAAUUCUAGGGAAAC | 9745 | AGUUUCCCUAGAAUUUUGC |
| | | U | | |
| siRNA 3392 | 3392 | CAAAAUUCUAGGGAAACU | 9746 | AAGUUUCCCUAGAAUUUU |
| | | U | | G |
| siRNA 3393 | 3393 | AAAAUUCUAGGGAAACUU | 9747 | CAAGUUUCCCUAGAAUUUU |
| | | G | | |
| siRNA 3394 | 3394 | AAAUUCUAGGGAAACUUG | 9748 | CCAAGUUUCCCUAGAAUUU |
| | | G | | |
| siRNA 3395 | 3395 | AAUUCUAGGGAAACUUGG | 9749 | GCCAAGUUUCCCUAGAAUU |
| | | C | | |
| siRNA 3396 | 3396 | AUUCUAGGGAAACUUGGCC | 9750 | GGCCAAGUUUCCCUAGAAU |
| siRNA 3397 | 3397 | UUCUAGGGAAACUUGGCCA | 9751 | UGGCCAAGUUUCCCUAGAA |
| siRNA 3398 | 3398 | UCUAGGGAAACUUGGCCAG | 9752 | CUGGCCAAGUUUCCCUAGA |
| siRNA 3399 | 3399 | CUAGGGAAACUUGGCCAGA | 9753 | UCUGGCCAAGUUUCCCUAG |
| siRNA 3400 | 3400 | UAGGGAAACUUGGCCAGA | 9754 | UUCUGGCCAAGUUUCCCUA |
| | | A | | |
| siRNA 3401 | 3401 | AGGGAAACUUGGCCAGAA | 9755 | UUUCUGGCCAAGUUUCCCU |
| | | A | | |
| siRNA 3402 | 3402 | GGGAAACUUGGCCAGAAA | 9756 | UUUUCUGGCCAAGUUUCCC |
| | | A | | |
| siRNA 3403 | 3403 | GGAAACUUGGCCAGAAAAC | 9757 | GUUUUCUGGCCAAGUUUCC |
| siRNA 3404 | 3404 | GAAACUUGGCCAGAAAACU | 9758 | AGUUUUCUGGCCAAGUUUC |
| siRNA 3405 | 3405 | AAACUUGGCCAGAAAACUG | 9759 | CAGUUUUCUGGCCAAGUUU |
| siRNA 3406 | 3406 | AACUUGGCCAGAAAACUGG | 9760 | CCAGUUUUCUGGCCAAGUU |
| siRNA 3407 | 3407 | ACUUGGCCAGAAAACUGGU | 9761 | ACCAGUUUUCUGGCCAAGU |
| siRNA 3408 | 3408 | CUUGGCCAGAAAACUGGUG | 9762 | CACCAGUUUUCUGGCCAAG |
| siRNA 3409 | 3409 | UUGGCCAGAAAACUGGUG | 9763 | ACACCAGUUUUCUGGCCAA |
| | | U | | |
| siRNA 3410 | 3410 | UGGCCAGAAAACUGGUGU | 9764 | AACACCAGUUUUCUGGCCA |
| | | U | | |
| siRNA 3411 | 3411 | GGCCAGAAAACUGGUGUU | 9765 | CAACACCAGUUUUCUGGCC |
| | | G | | |
| siRNA 3412 | 3412 | GCCAGAAAACUGGUGUUG | 9766 | UCAACACCAGUUUUCUGGC |
| | | A | | |
| siRNA 3413 | 3413 | CCAGAAAACUGGUGUUGA | 9767 | UUCAACACCAGUUUUCUGG |
| | | A | | |
| siRNA 3414 | 3414 | CAGAAAACUGGUGUUGAA | 9768 | CUUCAACACCAGUUUUCUG |
| | | G | | |
| siRNA 3415 | 3415 | AGAAAACUGGUGUUGAAG | 9769 | CCUUCAACACCAGUUUUCU |
| | | G | | |
| siRNA 3416 | 3416 | GAAAACUGGUGUUGAAGG | 9770 | GCCUUCAACACCAGUUUUC |
| | | C | | |
| siRNA 3417 | 3417 | AAAACUGGUGUUGAAGGC | 9771 | AGCCUUCAACACCAGUUUU |
| | | U | | |
| siRNA 3418 | 3418 | AAACUGGUGUUGAAGGCU | 9772 | AAGCCUUCAACACCAGUUU |
| | | U | | |
| siRNA 3419 | 3419 | AACUGGUGUUGAAGGCUU | 9773 | AAAGCCUUCAACACCAGUU |
| | | U | | |
| siRNA 3420 | 3420 | ACUGGUGUUGAAGGCUUU | 9774 | AAAAGCCUUCAACACCAGU |
| | | U | | |
| siRNA 3421 | 3421 | CUGGUGUUGAAGGCUUUU | 9775 | CAAAAGCCUUCAACACCAG |
| | | G | | |
| siRNA 3422 | 3422 | UGGUGUUGAAGGCUUUUG | 9776 | GCAAAAGCCUUCAACACCA |
| | | C | | |
| siRNA 3423 | 3423 | GGUGUUGAAGGCUUUUGC | 9777 | AGCAAAAGCCUUCAACACC |
| | | U | | |
| siRNA 3424 | 3424 | GUGUUGAAGGCUUUUGCU | 9778 | GAGCAAAAGCCUUCAACAC |
| | | C | | |
| siRNA 3425 | 3425 | UGUUGAAGGCUUUUGCUC | 9779 | UGAGCAAAAGCCUUCAACA |
| | | A | | |
| siRNA 3426 | 3426 | GUUGAAGGCUUUUGCUCA | 9780 | AUGAGCAAAAGCCUUCAAC |
| | | U | | |
| siRNA 3427 | 3427 | UUGAAGGCUUUUGCUCAU | 9781 | UAUGAGCAAAAGCCUUCAA |
| | | A | | |
| siRNA 3428 | 3428 | UGAAGGCUUUUGCUCAUA | 9782 | AUAUGAGCAAAAGCCUUCA |
| | | U | | |
| siRNA 3429 | 3429 | GAAGGCUUUUGCUCAUAU | 9783 | UAUAUGAGCAAAAGCCUUC |
| | | A | | |
| siRNA 3430 | 3430 | AAGGCUUUUGCUCAUAUA | 9784 | UUAUAUGAGCAAAAGCCU |
| | | A | | U |
| siRNA 3431 | 3431 | AGGCUUUUGCUCAUAUAA | 9785 | UUUAUAUGAGCAAAAGCC |
| | | A | | U |
| siRNA 3432 | 3432 | GGCUUUUGCUCAUAUAAAC | 9786 | GUUUAUAUGAGCAAAAGC |
| | | | | C |
| siRNA 3433 | 3433 | GCUUUUGCUCAUAUAAACA | 9787 | UGUUUAUAUGAGCAAAAG |
| | | | | C |
| siRNA 3434 | 3434 | CUUUUGCUCAUAUAAACAA | 9788 | UUGUUUAUAUGAGCAAAA |
| | | | | G |
| siRNA 3435 | 3435 | UUUUGCUCAUAUAAACAA | 9789 | CUUGUUUAUAUGAGCAAA |
| | | G | | A |
| siRNA 3436 | 3436 | UUUGCUCAUAUAAACAAG | 9790 | ACUUGUUUAUAUGAGCAA |
| | | U | | A |
| siRNA 3437 | 3437 | UUGCUCAUAUAAACAAGU | 9791 | CACUUGUUUAUAUGAGCA |
| | | G | | A |
| siRNA 3438 | 3438 | UGCUCAUAUAAACAAGUGC | 9792 | GCACUUGUUUAUAUGAGC |
| | | | | A |
| siRNA 3439 | 3439 | GCUCAUAUAAACAAGUGCC | 9793 | GGCACUUGUUUAUAUGAG |
| | | | | C |
| siRNA 3440 | 3440 | CUCAUAUAAACAAGUGCCA | 9794 | UGGCACUUGUUUAUAUGA |
| | | | | G |
| siRNA 3441 | 3441 | UCAUAUAAACAAGUGCCAU | 9795 | AUGGCACUUGUUUAUAUG |
| | | | | A |
| siRNA 3442 | 3442 | CAUAUAAACAAGUGCCAUU | 9796 | AAUGGCACUUGUUUAUAU |
| | | | | G |
| siRNA 3443 | 3443 | AUAUAAACAAGUGCCAUU | 9797 | CAAUGGCACUUGUUUAUA |
| | | G | | U |
| siRNA 3444 | 3444 | UAUAAACAAGUGCCAUUG | 9798 | UCAAUGGCACUUGUUUAU |
| | | A | | A |
| siRNA 3445 | 3445 | AUAAACAAGUGCCAUUGA | 9799 | CUCAAUGGCACUUGUUUAU |
| | | G | | |
| siRNA 3446 | 3446 | UAAACAAGUGCCAUUGAG | 9800 | ACUCAAUGGCACUUGUUUA |
| | | U | | |
| siRNA 3447 | 3447 | AAACAAGUGCCAUUGAGU | 9801 | AACUCAAUGGCACUUGUUU |
| | | U | | |
| siRNA 3448 | 3448 | AACAAGUGCCAUUGAGUU | 9802 | AAACUCAAUGGCACUUGUU |
| | | U | | |
| siRNA 3449 | 3449 | ACAAGUGCCAUUGAGUUUC | 9803 | GAAACUCAAUGGCACUUGU |
| siRNA 3450 | 3450 | CAAGUGCCAUUGAGUUUCA | 9804 | UGAAACUCAAUGGCACUUG |
| siRNA 3451 | 3451 | AAGUGCCAUUGAGUUUCA | 9805 | UUGAAACUCAAUGGCACUU |
| | | A | | |
| siRNA 3452 | 3452 | AGUGCCAUUGAGUUUCAA | 9806 | UUUGAAACUCAAUGGCACU |
| | | A | | |
| siRNA 3453 | 3453 | GUGCCAUUGAGUUUCAAA | 9807 | AUUUGAAACUCAAUGGCAC |
| | | U | | |
| siRNA 3454 | 3454 | UGCCAUUGAGUUUCAAAU | 9808 | CAUUUGAAACUCAAUGGCA |
| | | G | | |
| siRNA 3455 | 3455 | GCCAUUGAGUUUCAAAUG | 9809 | UCAUUUGAAACUCAAUGGC |
| | | A | | |
| siRNA 3456 | 3456 | CCAUUGAGUUUCAAAUGAC | 9810 | GUCAUUUGAAACUCAAUG |
| | | | | G |
| siRNA 3457 | 3457 | CAUUGAGUUUCAAAUGACC | 9811 | GGUCAUUUGAAACUCAAU |
| | | | | G |
| siRNA 3458 | 3458 | AUUGAGUUUCAAAUGACC | 9812 | UGGUCAUUUGAAACUCAA |
| | | A | | U |
| siRNA 3459 | 3459 | UUGAGUUUCAAAUGACCA | 9813 | CUGGUCAUUUGAAACUCAA |
| | | G | | |
| siRNA 3460 | 3460 | UGAGUUUCAAAUGACCAGC | 9814 | GCUGGUCAUUUGAAACUCA |
| siRNA 3461 | 3461 | GAGUUUCAAAUGACCAGCA | 9815 | UGCUGGUCAUUUGAAACUC |
| siRNA 3462 | 3462 | AGUUUCAAAUGACCAGCAA | 9816 | UUGCUGGUCAUUUGAAAC |
| | | | | U |
| siRNA 3463 | 3463 | GUUUCAAAUGACCAGCAAA | 9817 | UUUGCUGGUCAUUUGAAA |
| | | | | C |
| siRNA 3464 | 3464 | UUUCAAAUGACCAGCAAAU | 9818 | AUUUGCUGGUCAUUUGAA |
| | | | | A |
| siRNA 3465 | 3465 | UUCAAAUGACCAGCAAAUA | 9819 | UAUUUGCUGGUCAUUUGA |
| | | | | A |
| siRNA 3466 | 3466 | UCAAAUGACCAGCAAAUAU | 9820 | AUAUUUGCUGGUCAUUUG |
| | | | | A |
| siRNA 3467 | 3467 | CAAAUGACCAGCAAAUAUA | 9821 | UAUAUUUGCUGGUCAUUU |
| | | | | G |
| siRNA 3468 | 3468 | AAAUGACCAGCAAAUAUA | 9822 | AUAUAUUUGCUGGUCAUU |
| | | U | | U |
| siRNA 3469 | 3469 | AAUGACCAGCAAAUAUAU | 9823 | AAUAUAUUUGCUGGUCAU |
| | | U | | U |
| siRNA 3470 | 3470 | AUGACCAGCAAAUAUAUU | 9824 | AAAUAUAUUUGCUGGUCA |
| | | U | | U |
| siRNA 3471 | 3471 | UGACCAGCAAAUAUAUUU | 9825 | UAAAUAUAUUUGCUGGUC |
| | | A | | A |
| siRNA 3472 | 3472 | GACCAGCAAAUAUAUUUA | 9826 | CUAAAUAUAUUUGCUGGU |
| | | G | | C |
| siRNA 3473 | 3473 | ACCAGCAAAUAUAUUUAG | 9827 | UCUAAAUAUAUUUGCUGG |
| | | A | | U |
| siRNA 3474 | 3474 | CCAGCAAAUAUAUUUAGA | 9828 | UUCUAAAUAUAUUUGCUG |
| | | A | | G |
| siRNA 3475 | 3475 | CAGCAAAUAUAUUUAGAA | 9829 | GUUCUAAAUAUAUUUGCU |
| | | C | | G |
| siRNA 3476 | 3476 | AGCAAAUAUAUUUAGAAC | 9830 | GGUUCUAAAUAUAUUUGC |
| | | C | | U |
| siRNA 3477 | 3477 | GCAAAUAUAUUUAGAACCC | 9831 | GGGUUCUAAAUAUAUUUG |
| | | | | C |
| siRNA 3478 | 3478 | CAAAUAUAUUUAGAACCCU | 9832 | AGGGUUCUAAAUAUAUUU |
| | | | | G |
| siRNA 3479 | 3479 | AAAUAUAUUUAGAACCCU | 9833 | AAGGGUUCUAAAUAUAUU |
| | | U | | U |
| siRNA 3480 | 3480 | AAUAUAUUUAGAACCCUUC | 9834 | GAAGGGUUCUAAAUAUAU |
| | | | | U |
| siRNA 3481 | 3481 | AUAUAUUUAGAACCCUUCC | 9835 | GGAAGGGUUCUAAAUAUA |
| | | | | U |
| siRNA 3482 | 3482 | UAUAUUUAGAACCCUUCCU | 9836 | AGGAAGGGUUCUAAAUAU |
| | | | | A |
| siRNA 3483 | 3483 | AUAUUUAGAACCCUUCCUG | 9837 | CAGGAAGGGUUCUAAAUA |
| | | | | U |
| siRNA 3484 | 3484 | UAUUUAGAACCCUUCCUGU | 9838 | ACAGGAAGGGUUCUAAAU |
| | | | | A |
| siRNA 3485 | 3485 | AUUUAGAACCCUUCCUGUU | 9839 | AACAGGAAGGGUUCUAAA |
| | | | | U |
| siRNA 3486 | 3486 | UUUAGAACCCUUCCUGUUU | 9840 | AAACAGGAAGGGUUCUAA |
| | | | | A |
| siRNA 3487 | 3487 | UUAGAACCCUUCCUGUUUU | 9841 | AAAACAGGAAGGGUUCUA |
| | | | | A |
| siRNA 3488 | 3488 | UAGAACCCUUCCUGUUUUA | 9842 | UAAAACAGGAAGGGUUCU |
| | | | | A |
| siRNA 3489 | 3489 | AGAACCCUUCCUGUUUUAU | 9843 | AUAAAACAGGAAGGGUUC |
| | | | | U |
| siRNA 3490 | 3490 | GAACCCUUCCUGUUUUAUG | 9844 | CAUAAAACAGGAAGGGUU |
| | | | | C |
| siRNA 3491 | 3491 | AACCCUUCCUGUUUUAUGU | 9845 | ACAUAAAACAGGAAGGGU |
| | | | | U |
| siRNA 3492 | 3492 | ACCCUUCCUGUUUUAUGUC | 9846 | GACAUAAAACAGGAAGGG |
| | | | | U |
| siRNA 3493 | 3493 | CCCUUCCUGUUUUAUGUCU | 9847 | AGACAUAAAACAGGAAGG |
| | | | | G |
| siRNA 3494 | 3494 | CCUUCCUGUUUUAUGUCUG | 9848 | CAGACAUAAAACAGGAAG |
| | | | | G |
| siRNA 3495 | 3495 | CUUCCUGUUUUAUGUCUGU | 9849 | ACAGACAUAAAACAGGAA |
| | | | | G |
| siRNA 3496 | 3496 | UUCCUGUUUUAUGUCUGU | 9850 | UACAGACAUAAAACAGGA |
| | | A | | A |
| siRNA 3497 | 3497 | UCCUGUUUUAUGUCUGUAC | 9851 | GUACAGACAUAAAACAGG |
| | | | | A |
| siRNA 3498 | 3498 | CCUGUUUUAUGUCUGUACC | 9852 | GGUACAGACAUAAAACAG |
| | | | | G |
| siRNA 3499 | 3499 | CUGUUUUAUGUCUGUACCU | 9853 | AGGUACAGACAUAAAACA |
| | | | | G |
| siRNA 3500 | 3500 | UGUUUUAUGUCUGUACCUC | 9854 | GAGGUACAGACAUAAAAC |
| | | | | A |
| siRNA 3501 | 3501 | GUUUUAUGUCUGUACCUCG | 9855 | CGAGGUACAGACAUAAAAC |
| siRNA 3502 | 3502 | UUUUAUGUCUGUACCUCGU | 9856 | ACGAGGUACAGACAUAAA |
| | | | | A |
| siRNA 3503 | 3503 | UUUAUGUCUGUACCUCGUC | 9857 | GACGAGGUACAGACAUAA |
| | | | | A |
| siRNA 3504 | 3504 | UUAUGUCUGUACCUCGUCC | 9858 | GGACGAGGUACAGACAUA |
| | | | | A |
| siRNA 3505 | 3505 | UAUGUCUGUACCUCGUCCA | 9859 | UGGACGAGGUACAGACAU |
| | | | | A |
| siRNA 3506 | 3506 | AUGUCUGUACCUCGUCCAC | 9860 | GUGGACGAGGUACAGACA |
| | | | | U |
| siRNA 3507 | 3507 | UGUCUGUACCUCGUCCACC | 9861 | GGUGGACGAGGUACAGAC |
| | | | | A |
| siRNA 3508 | 3508 | GUCUGUACCUCGUCCACCC | 9862 | GGGUGGACGAGGUACAGA |
| | | | | C |
| siRNA 3509 | 3509 | UCUGUACCUCGUCCACCCC | 9863 | GGGGUGGACGAGGUACAG |
| | | | | A |
| siRNA 3510 | 3510 | CUGUACCUCGUCCACCCCU | 9864 | AGGGGUGGACGAGGUACA |
| | | | | G |
| siRNA 3511 | 3511 | UGUACCUCGUCCACCCCUC | 9865 | GAGGGGUGGACGAGGUAC |
| | | | | A |
| siRNA 3512 | 3512 | GUACCUCGUCCACCCCUCA | 9866 | UGAGGGGUGGACGAGGUA |
| | | | | C |
| siRNA 3513 | 3513 | UACCUCGUCCACCCCUCAG | 9867 | CUGAGGGGUGGACGAGGU |
| | | | | A |
| siRNA 3514 | 3514 | ACCUCGUCCACCCCUCAGG | 9868 | CCUGAGGGGUGGACGAGG |
| | | | | U |
| siRNA 3515 | 3515 | CCUCGUCCACCCCUCAGGU | 9869 | ACCUGAGGGGUGGACGAG |
| | | | | G |
| siRNA 3516 | 3516 | CUCGUCCACCCCUCAGGUA | 9870 | UACCUGAGGGGUGGACGA |
| | | | | G |
| siRNA 3517 | 3517 | UCGUCCACCCCUCAGGUAA | 9871 | UUACCUGAGGGGUGGACG |
| | | | | A |
| siRNA 3518 | 3518 | CGUCCACCCCUCAGGUAAU | 9872 | AUUACCUGAGGGGUGGAC |
| | | | | G |
| siRNA 3519 | 3519 | GUCCACCCCUCAGGUAAUA | 9873 | UAUUACCUGAGGGGUGGA |
| | | | | C |
| siRNA 3520 | 3520 | UCCACCCCUCAGGUAAUAC | 9874 | GUAUUACCUGAGGGGUGG |
| | | | | A |
| siRNA 3521 | 3521 | CCACCCCUCAGGUAAUACC | 9875 | GGUAUUACCUGAGGGGUG |
| | | | | G |
| siRNA 3522 | 3522 | CACCCCUCAGGUAAUACCU | 9876 | AGGUAUUACCUGAGGGGU |
| | | | | G |
| siRNA 3523 | 3523 | ACCCCUCAGGUAAUACCUG | 9877 | CAGGUAUUACCUGAGGGG |
| | | | | U |
| siRNA 3524 | 3524 | CCCCUCAGGUAAUACCUGC | 9878 | GCAGGUAUUACCUGAGGG |
| | | | | G |
| siRNA 3525 | 3525 | CCCUCAGGUAAUACCUGCC | 9879 | GGCAGGUAUUACCUGAGG |
| | | | | G |
| siRNA 3526 | 3526 | CCUCAGGUAAUACCUGCCU | 9880 | AGGCAGGUAUUACCUGAG |
| | | | | G |
| siRNA 3527 | 3527 | CUCAGGUAAUACCUGCCUC | 9881 | GAGGCAGGUAUUACCUGA |
| | | | | G |
| siRNA 3528 | 3528 | UCAGGUAAUACCUGCCUCU | 9882 | AGAGGCAGGUAUUACCUG |
| | | | | A |
| siRNA 3529 | 3529 | CAGGUAAUACCUGCCUCUC | 9883 | GAGAGGCAGGUAUUACCU |
| | | | | G |
| siRNA 3530 | 3530 | AGGUAAUACCUGCCUCUCA | 9884 | UGAGAGGCAGGUAUUACC |
| | | | | U |
| siRNA 3531 | 3531 | GGUAAUACCUGCCUCUCAC | 9885 | GUGAGAGGCAGGUAUUAC |
| | | | | C |
| siRNA 3532 | 3532 | GUAAUACCUGCCUCUCACA | 9886 | UGUGAGAGGCAGGUAUUA |
| | | | | C |
| siRNA 3533 | 3533 | UAAUACCUGCCUCUCACAG | 9887 | CUGUGAGAGGCAGGUAUU |
| | | | | A |
| siRNA 3534 | 3534 | AAUACCUGCCUCUCACAGG | 9888 | CCUGUGAGAGGCAGGUAU |
| | | | | U |
| siRNA 3535 | 3535 | AUACCUGCCUCUCACAGGU | 9889 | ACCUGUGAGAGGCAGGUA |
| | | | | U |
| siRNA 3536 | 3536 | UACCUGCCUCUCACAGGUA | 9890 | UACCUGUGAGAGGCAGGU |
| | | | | A |
| siRNA 3537 | 3537 | ACCUGCCUCUCACAGGUAC | 9891 | GUACCUGUGAGAGGCAGG |
| | | | | U |
| siRNA 3538 | 3538 | CCUGCCUCUCACAGGUACA | 9892 | UGUACCUGUGAGAGGCAG |
| | | | | G |
| siRNA 3539 | 3539 | CUGCCUCUCACAGGUACAG | 9893 | CUGUACCUGUGAGAGGCAG |
| siRNA 3540 | 3540 | UGCCUCUCACAGGUACAGC | 9894 | GCUGUACCUGUGAGAGGCA |
| siRNA 3541 | 3541 | GCCUCUCACAGGUACAGCU | 9895 | AGCUGUACCUGUGAGAGGC |
| siRNA 3542 | 3542 | CCUCUCACAGGUACAGCUG | 9896 | CAGCUGUACCUGUGAGAGG |
| siRNA 3543 | 3543 | CUCUCACAGGUACAGCUGU | 9897 | ACAGCUGUACCUGUGAGAG |
| siRNA 3544 | 3544 | UCUCACAGGUACAGCUGUU | 9898 | AACAGCUGUACCUGUGAGA |
| siRNA 3545 | 3545 | CUCACAGGUACAGCUGUUU | 9899 | AAACAGCUGUACCUGUGAG |
| siRNA 3546 | 3546 | UCACAGGUACAGCUGUUUC | 9900 | GAAACAGCUGUACCUGUGA |
| siRNA 3547 | 3547 | CACAGGUACAGCUGUUUCU | 9901 | AGAAACAGCUGUACCUGUG |
| siRNA 3548 | 3548 | ACAGGUACAGCUGUUUCUU | 9902 | AAGAAACAGCUGUACCUGU |
| siRNA 3549 | 3549 | CAGGUACAGCUGUUUCUUG | 9903 | CAAGAAACAGCUGUACCUG |
| siRNA 3550 | 3550 | AGGUACAGCUGUUUCUUG | 9904 | CCAAGAAACAGCUGUACCU |
| | | G | | |
| siRNA 3551 | 3551 | GGUACAGCUGUUUCUUGG | 9905 | UCCAAGAAACAGCUGUACC |
| | | A | | |
| siRNA 3552 | 3552 | GUACAGCUGUUUCUUGGA | 9906 | UUCCAAGAAACAGCUGUAC |
| | | A | | |
| siRNA 3553 | 3553 | UACAGCUGUUUCUUGGAA | 9907 | UUUCCAAGAAACAGCUGUA |
| | | A | | |
| siRNA 3554 | 3554 | ACAGCUGUUUCUUGGAAA | 9908 | AUUUCCAAGAAACAGCUGU |
| | | U | | |
| siRNA 3555 | 3555 | CAGCUGUUUCUUGGAAAUC | 9909 | GAUUUCCAAGAAACAGCUG |
| siRNA 3556 | 3556 | AGCUGUUUCUUGGAAAUCC | 9910 | GGAUUUCCAAGAAACAGCU |
| siRNA 3557 | 3557 | GCUGUUUCUUGGAAAUCCU | 9911 | AGGAUUUCCAAGAAACAGC |
| siRNA 3558 | 3558 | CUGUUUCUUGGAAAUCCUC | 9912 | GAGGAUUUCCAAGAAACA |
| | | | | G |
| siRNA 3559 | 3559 | UGUUUCUUGGAAAUCCUCC | 9913 | GGAGGAUUUCCAAGAAAC |
| | | | | A |
| siRNA 3560 | 3560 | GUUUCUUGGAAAUCCUCCA | 9914 | UGGAGGAUUUCCAAGAAA |
| | | | | C |
| siRNA 3561 | 3561 | UUUCUUGGAAAUCCUCCAA | 9915 | UUGGAGGAUUUCCAAGAA |
| | | | | A |
| siRNA 3562 | 3562 | UUCUUGGAAAUCCUCCAAC | 9916 | GUUGGAGGAUUUCCAAGA |
| | | | | A |
| siRNA 3563 | 3563 | UCUUGGAAAUCCUCCAACC | 9917 | GGUUGGAGGAUUUCCAAG |
| | | | | A |
| siRNA 3564 | 3564 | CUUGGAAAUCCUCCAACCA | 9918 | UGGUUGGAGGAUUUCCAA |
| | | | | G |
| siRNA 3565 | 3565 | UUGGAAAUCCUCCAACCAA | 9919 | UUGGUUGGAGGAUUUCCA |
| | | | | A |
| siRNA 3566 | 3566 | UGGAAAUCCUCCAACCAAA | 9920 | UUUGGUUGGAGGAUUUCC |
| | | | | A |
| siRNA 3567 | 3567 | GGAAAUCCUCCAACCAAAU | 9921 | AUUUGGUUGGAGGAUUUC |
| | | | | C |
| siRNA 3568 | 3568 | GAAAUCCUCCAACCAAAUA | 9922 | UAUUUGGUUGGAGGAUUU |
| | | | | C |
| siRNA 3569 | 3569 | AAAUCCUCCAACCAAAUAG | 9923 | CUAUUUGGUUGGAGGAUU |
| | | | | U |
| siRNA 3570 | 3570 | AAUCCUCCAACCAAAUAGC | 9924 | GCUAUUUGGUUGGAGGAU |
| | | | | U |
| siRNA 3571 | 3571 | AUCCUCCAACCAAAUAGCA | 9925 | UGCUAUUUGGUUGGAGGA |
| | | | | U |
| siRNA 3572 | 3572 | UCCUCCAACCAAAUAGCAG | 9926 | CUGCUAUUUGGUUGGAGG |
| | | | | A |
| siRNA 3573 | 3573 | CCUCCAACCAAAUAGCAGU | 9927 | ACUGCUAUUUGGUUGGAG |
| | | | | G |
| siRNA 3574 | 3574 | CUCCAACCAAAUAGCAGUU | 9928 | AACUGCUAUUUGGUUGGA |
| | | | | G |
| siRNA 3575 | 3575 | UCCAACCAAAUAGCAGUUU | 9929 | AAACUGCUAUUUGGUUGG |
| | | | | A |
| siRNA 3576 | 3576 | CCAACCAAAUAGCAGUUUU | 9930 | AAAACUGCUAUUUGGUUG |
| | | | | G |
| siRNA 3577 | 3577 | CAACCAAAUAGCAGUUUUC | 9931 | GAAAACUGCUAUUUGGUU |
| | | | | G |
| siRNA 3578 | 3578 | AACCAAAUAGCAGUUUUCC | 9932 | GGAAAACUGCUAUUUGGU |
| | | | | U |
| siRNA 3579 | 3579 | ACCAAAUAGCAGUUUUCCU | 9933 | AGGAAAACUGCUAUUUGG |
| | | | | U |
| siRNA 3580 | 3580 | CCAAAUAGCAGUUUUCCUA | 9934 | UAGGAAAACUGCUAUUUG |
| | | | | G |
| siRNA 3581 | 3581 | CAAAUAGCAGUUUUCCUAA | 9935 | UUAGGAAAACUGCUAUUU |
| | | | | G |
| siRNA 3582 | 3582 | AAAUAGCAGUUUUCCUAAC | 9936 | GUUAGGAAAACUGCUAUU |
| | | | | U |
| siRNA 3583 | 3583 | AAUAGCAGUUUUCCUAACU | 9937 | AGUUAGGAAAACUGCUAU |
| | | | | U |
| siRNA 3584 | 3584 | AUAGCAGUUUUCCUAACUU | 9938 | AAGUUAGGAAAACUGCUA |
| | | | | U |
| siRNA 3585 | 3585 | UAGCAGUUUUCCUAACUUG | 9939 | CAAGUUAGGAAAACUGCU |
| | | | | A |
| siRNA 3586 | 3586 | AGCAGUUUUCCUAACUUGA | 9940 | UCAAGUUAGGAAAACUGC |
| | | | | U |
| siRNA 3587 | 3587 | GCAGUUUUCCUAACUUGAU | 9941 | AUCAAGUUAGGAAAACUG |
| | | | | C |
| siRNA 3588 | 3588 | CAGUUUUCCUAACUUGAUU | 9942 | AAUCAAGUUAGGAAAACU |
| | | | | G |
| siRNA 3589 | 3589 | AGUUUUCCUAACUUGAUU | 9943 | UAAUCAAGUUAGGAAAAC |
| | | A | | U |
| siRNA 3590 | 3590 | GUUUUCCUAACUUGAUUA | 9944 | CUAAUCAAGUUAGGAAAA |
| | | G | | C |
| siRNA 3591 | 3591 | UUUUCCUAACUUGAUUAGC | 9945 | GCUAAUCAAGUUAGGAAA |
| | | | | A |
| siRNA 3592 | 3592 | UUUCCUAACUUGAUUAGCU | 9946 | AGCUAAUCAAGUUAGGAA |
| | | | | A |
| siRNA 3593 | 3593 | UUCCUAACUUGAUUAGCUU | 9947 | AAGCUAAUCAAGUUAGGA |
| | | | | A |
| siRNA 3594 | 3594 | UCCUAACUUGAUUAGCUUG | 9948 | CAAGCUAAUCAAGUUAGG |
| | | | | A |
| siRNA 3595 | 3595 | CCUAACUUGAUUAGCUUGA | 9949 | UCAAGCUAAUCAAGUUAG |
| | | | | G |
| siRNA 3596 | 3596 | CUAACUUGAUUAGCUUGA | 9950 | CUCAAGCUAAUCAAGUUAG |
| | | G | | |
| siRNA 3597 | 3597 | UAACUUGAUUAGCUUGAG | 9951 | GCUCAAGCUAAUCAAGUUA |
| | | C | | |
| siRNA 3598 | 3598 | AACUUGAUUAGCUUGAGC | 9952 | AGCUCAAGCUAAUCAAGUU |
| | | U | | |
| siRNA 3599 | 3599 | ACUUGAUUAGCUUGAGCU | 9953 | CAGCUCAAGCUAAUCAAGU |
| | | G | | |
| siRNA 3600 | 3600 | CUUGAUUAGCUUGAGCUG | 9954 | UCAGCUCAAGCUAAUCAAG |
| | | A | | |
| siRNA 3601 | 3601 | UUGAUUAGCUUGAGCUGA | 9955 | GUCAGCUCAAGCUAAUCAA |
| | | C | | |
| siRNA 3602 | 3602 | UGAUUAGCUUGAGCUGAC | 9956 | UGUCAGCUCAAGCUAAUCA |
| | | A | | |
| siRNA 3603 | 3603 | GAUUAGCUUGAGCUGACA | 9957 | CUGUCAGCUCAAGCUAAUC |
| | | G | | |
| siRNA 3604 | 3604 | AUUAGCUUGAGCUGACAG | 9958 | UCUGUCAGCUCAAGCUAAU |
| | | A | | |
| siRNA 3605 | 3605 | UUAGCUUGAGCUGACAGAC | 9959 | GUCUGUCAGCUCAAGCUAA |
| siRNA 3606 | 3606 | UAGCUUGAGCUGACAGACU | 9960 | AGUCUGUCAGCUCAAGCUA |
| siRNA 3607 | 3607 | AGCUUGAGCUGACAGACUG | 9961 | CAGUCUGUCAGCUCAAGCU |
| siRNA 3608 | 3608 | GCUUGAGCUGACAGACUGU | 9962 | ACAGUCUGUCAGCUCAAGC |
| siRNA 3609 | 3609 | CUUGAGCUGACAGACUGUU | 9963 | AACAGUCUGUCAGCUCAAG |
| siRNA 3610 | 3610 | UUGAGCUGACAGACUGUU | 9964 | UAACAGUCUGUCAGCUCAA |
| | | A | | |
| siRNA 3611 | 3611 | UGAGCUGACAGACUGUUA | 9965 | CUAACAGUCUGUCAGCUCA |
| | | G | | |
| siRNA 3612 | 3612 | GAGCUGACAGACUGUUAG | 9966 | UCUAACAGUCUGUCAGCUC |
| | | A | | |
| siRNA 3613 | 3613 | AGCUGACAGACUGUUAGA | 9967 | UUCUAACAGUCUGUCAGCU |
| | | A | | |
| siRNA 3614 | 3614 | GCUGACAGACUGUUAGAA | 9968 | AUUCUAACAGUCUGUCAGC |
| | | U | | |
| siRNA 3615 | 3615 | CUGACAGACUGUUAGAAU | 9969 | UAUUCUAACAGUCUGUCAG |
| | | A | | |
| siRNA 3616 | 3616 | UGACAGACUGUUAGAAUA | 9970 | GUAUUCUAACAGUCUGUCA |
| | | C | | |
| siRNA 3617 | 3617 | GACAGACUGUUAGAAUAC | 9971 | UGUAUUCUAACAGUCUGUC |
| | | A | | |
| siRNA 3618 | 3618 | ACAGACUGUUAGAAUACA | 9972 | CUGUAUUCUAACAGUCUGU |
| | | G | | |
| siRNA 3619 | 3619 | CAGACUGUUAGAAUACAG | 9973 | ACUGUAUUCUAACAGUCUG |
| | | U | | |
| siRNA 3620 | 3620 | AGACUGUUAGAAUACAGU | 9974 | AACUGUAUUCUAACAGUCU |
| | | U | | |
| siRNA 3621 | 3621 | GACUGUUAGAAUACAGUU | 9975 | GAACUGUAUUCUAACAGUC |
| | | C | | |
| siRNA 3622 | 3622 | ACUGUUAGAAUACAGUUC | 9976 | AGAACUGUAUUCUAACAG |
| | | U | | U |
| siRNA 3623 | 3623 | CUGUUAGAAUACAGUUCUC | 9977 | GAGAACUGUAUUCUAACA |
| | | | | G |
| siRNA 3624 | 3624 | UGUUAGAAUACAGUUCUC | 9978 | AGAGAACUGUAUUCUAAC |
| | | U | | A |
| siRNA 3625 | 3625 | GUUAGAAUACAGUUCUCU | 9979 | CAGAGAACUGUAUUCUAAC |
| | | G | | |
| siRNA 3626 | 3626 | UUAGAAUACAGUUCUCUG | 9980 | CCAGAGAACUGUAUUCUAA |
| | | G | | |
| siRNA 3627 | 3627 | UAGAAUACAGUUCUCUGGC | 9981 | GCCAGAGAACUGUAUUCUA |
| siRNA 3628 | 3628 | AGAAUACAGUUCUCUGGCC | 9982 | GGCCAGAGAACUGUAUUCU |
| siRNA 3629 | 3629 | GAAUACAGUUCUCUGGCCA | 9983 | UGGCCAGAGAACUGUAUUC |
| siRNA 3630 | 3630 | AAUACAGUUCUCUGGCCAC | 9984 | GUGGCCAGAGAACUGUAU |
| | | | | U |
| siRNA 3631 | 3631 | AUACAGUUCUCUGGCCACA | 9985 | UGUGGCCAGAGAACUGUA |
| | | | | U |
| siRNA 3632 | 3632 | UACAGUUCUCUGGCCACAG | 9986 | CUGUGGCCAGAGAACUGUA |
| siRNA 3633 | 3633 | ACAGUUCUCUGGCCACAGC | 9987 | GCUGUGGCCAGAGAACUGU |
| siRNA 3634 | 3634 | CAGUUCUCUGGCCACAGCU | 9988 | AGCUGUGGCCAGAGAACUG |
| siRNA 3635 | 3635 | AGUUCUCUGGCCACAGCUG | 9989 | CAGCUGUGGCCAGAGAACU |
| siRNA 3636 | 3636 | GUUCUCUGGCCACAGCUGA | 9990 | UCAGCUGUGGCCAGAGAAC |
| siRNA 3637 | 3637 | UUCUCUGGCCACAGCUGAU | 9991 | AUCAGCUGUGGCCAGAGAA |
| siRNA 3638 | 3638 | UCUCUGGCCACAGCUGAUG | 9992 | CAUCAGCUGUGGCCAGAGA |
| siRNA 3639 | 3639 | CUCUGGCCACAGCUGAUGA | 9993 | UCAUCAGCUGUGGCCAGAG |
| siRNA 3640 | 3640 | UCUGGCCACAGCUGAUGAG | 9994 | CUCAUCAGCUGUGGCCAGA |
| siRNA 3641 | 3641 | CUGGCCACAGCUGAUGAGG | 9995 | CCUCAUCAGCUGUGGCCAG |
| siRNA 3642 | 3642 | UGGCCACAGCUGAUGAGGG | 9996 | CCCUCAUCAGCUGUGGCCA |
| siRNA 3643 | 3643 | GGCCACAGCUGAUGAGGGC | 9997 | GCCCUCAUCAGCUGUGGCC |
| siRNA 3644 | 3644 | GCCACAGCUGAUGAGGGCU | 9998 | AGCCCUCAUCAGCUGUGGC |
| siRNA 3645 | 3645 | CCACAGCUGAUGAGGGCUU | 9999 | AAGCCCUCAUCAGCUGUGG |
| siRNA 3646 | 3646 | CACAGCUGAUGAGGGCUUU | 10000 | AAAGCCCUCAUCAGCUGUG |
| siRNA 3647 | 3647 | ACAGCUGAUGAGGGCUUUC | 10001 | GAAAGCCCUCAUCAGCUGU |
| siRNA 3648 | 3648 | CAGCUGAUGAGGGCUUUCU | 10002 | AGAAAGCCCUCAUCAGCUG |
| siRNA 3649 | 3649 | AGCUGAUGAGGGCUUUCU | 10003 | CAGAAAGCCCUCAUCAGCU |
| | | G | | |
| siRNA 3650 | 3650 | GCUGAUGAGGGCUUUCUG | 10004 | ACAGAAAGCCCUCAUCAGC |
| | | U | | |
| siRNA 3651 | 3651 | CUGAUGAGGGCUUUCUGU | 10005 | UACAGAAAGCCCUCAUCAG |
| | | A | | |
| siRNA 3652 | 3652 | UGAUGAGGGCUUUCUGUA | 10006 | GUACAGAAAGCCCUCAUCA |
| | | C | | |
| siRNA 3653 | 3653 | GAUGAGGGCUUUCUGUAC | 10007 | AGUACAGAAAGCCCUCAUC |
| | | U | | |
| siRNA 3654 | 3654 | AUGAGGGCUUUCUGUACU | 10008 | CAGUACAGAAAGCCCUCAU |
| | | G | | |
| siRNA 3655 | 3655 | UGAGGGCUUUCUGUACUGC | 10009 | GCAGUACAGAAAGCCCUCA |
| siRNA 3656 | 3656 | GAGGGCUUUCUGUACUGCA | 10010 | UGCAGUACAGAAAGCCCUC |
| siRNA 3657 | 3657 | AGGGCUUUCUGUACUGCAC | 10011 | GUGCAGUACAGAAAGCCCU |
| siRNA 3658 | 3658 | GGGCUUUCUGUACUGCACA | 10012 | UGUGCAGUACAGAAAGCCC |
| siRNA 3659 | 3659 | GGCUUUCUGUACUGCACAC | 10013 | GUGUGCAGUACAGAAAGCC |
| siRNA 3660 | 3660 | GCUUUCUGUACUGCACACA | 10014 | UGUGUGCAGUACAGAAAG |
| | | | | C |
| siRNA 3661 | 3661 | CUUUCUGUACUGCACACAG | 10015 | CUGUGUGCAGUACAGAAA |
| | | | | G |
| siRNA 3662 | 3662 | UUUCUGUACUGCACACAGA | 10016 | UCUGUGUGCAGUACAGAA |
| | | | | A |
| siRNA 3663 | 3663 | UUCUGUACUGCACACAGAU | 10017 | AUCUGUGUGCAGUACAGA |
| | | | | A |
| siRNA 3664 | 3664 | UCUGUACUGCACACAGAUU | 10018 | AAUCUGUGUGCAGUACAG |
| | | | | A |
| siRNA 3665 | 3665 | CUGUACUGCACACAGAUUG | 10019 | CAAUCUGUGUGCAGUACAG |
| siRNA 3666 | 3666 | UGUACUGCACACAGAUUGU | 10020 | ACAAUCUGUGUGCAGUACA |
| siRNA 3667 | 3667 | GUACUGCACACAGAUUGUG | 10021 | CACAAUCUGUGUGCAGUAC |
| siRNA 3668 | 3668 | UACUGCACACAGAUUGUGU | 10022 | ACACAAUCUGUGUGCAGUA |
| siRNA 3669 | 3669 | ACUGCACACAGAUUGUGUA | 10023 | UACACAAUCUGUGUGCAGU |
| siRNA 3670 | 3670 | CUGCACACAGAUUGUGUAC | 10024 | GUACACAAUCUGUGUGCAG |
| siRNA 3671 | 3671 | UGCACACAGAUUGUGUACU | 10025 | AGUACACAAUCUGUGUGCA |
| siRNA 3672 | 3672 | GCACACAGAUUGUGUACUG | 10026 | CAGUACACAAUCUGUGUGC |
| siRNA 3673 | 3673 | CACACAGAUUGUGUACUGC | 10027 | GCAGUACACAAUCUGUGUG |
| siRNA 3674 | 3674 | ACACAGAUUGUGUACUGCA | 10028 | UGCAGUACACAAUCUGUGU |
| siRNA 3675 | 3675 | CACAGAUUGUGUACUGCAC | 10029 | GUGCAGUACACAAUCUGUG |
| siRNA 3676 | 3676 | ACAGAUUGUGUACUGCACC | 10030 | GGUGCAGUACACAAUCUGU |
| siRNA 3677 | 3677 | CAGAUUGUGUACUGCACCC | 10031 | GGGUGCAGUACACAAUCUG |
| siRNA 3678 | 3678 | AGAUUGUGUACUGCACCCC | 10032 | GGGGUGCAGUACACAAUCU |
| siRNA 3679 | 3679 | GAUUGUGUACUGCACCCCA | 10033 | UGGGGUGCAGUACACAAUC |
| siRNA 3680 | 3680 | AUUGUGUACUGCACCCCAG | 10034 | CUGGGGUGCAGUACACAAU |
| siRNA 3681 | 3681 | UUGUGUACUGCACCCCAGU | 10035 | ACUGGGGUGCAGUACACAA |
| siRNA 3682 | 3682 | UGUGUACUGCACCCCAGUC | 10036 | GACUGGGGUGCAGUACACA |
| siRNA 3683 | 3683 | GUGUACUGCACCCCAGUCC | 10037 | GGACUGGGGUGCAGUACAC |
| siRNA 3684 | 3684 | UGUACUGCACCCCAGUCCA | 10038 | UGGACUGGGGUGCAGUAC |
| | | | | A |
| siRNA 3685 | 3685 | GUACUGCACCCCAGUCCAG | 10039 | CUGGACUGGGGUGCAGUAC |
| siRNA 3686 | 3686 | UACUGCACCCCAGUCCAGG | 10040 | CCUGGACUGGGGUGCAGUA |
| siRNA 3687 | 3687 | ACUGCACCCCAGUCCAGGU | 10041 | ACCUGGACUGGGGUGCAGU |
| siRNA 3688 | 3688 | CUGCACCCCAGUCCAGGUG | 10042 | CACCUGGACUGGGGUGCAG |
| siRNA 3689 | 3689 | UGCACCCCAGUCCAGGUGA | 10043 | UCACCUGGACUGGGGUGCA |
| siRNA 3690 | 3690 | GCACCCCAGUCCAGGUGAC | 10044 | GUCACCUGGACUGGGGUGC |
| siRNA 3691 | 3691 | CACCCCAGUCCAGGUGACU | 10045 | AGUCACCUGGACUGGGGUG |
| siRNA 3692 | 3692 | ACCCCAGUCCAGGUGACUG | 10046 | CAGUCACCUGGACUGGGGU |
| siRNA 3693 | 3693 | CCCCAGUCCAGGUGACUGG | 10047 | CCAGUCACCUGGACUGGGG |
| siRNA 3694 | 3694 | CCCAGUCCAGGUGACUGGU | 10048 | ACCAGUCACCUGGACUGGG |
| siRNA 3695 | 3695 | CCAGUCCAGGUGACUGGUA | 10049 | UACCAGUCACCUGGACUGG |
| siRNA 3696 | 3696 | CAGUCCAGGUGACUGGUAC | 10050 | GUACCAGUCACCUGGACUG |
| siRNA 3697 | 3697 | AGUCCAGGUGACUGGUACC | 10051 | GGUACCAGUCACCUGGACU |
| siRNA 3698 | 3698 | GUCCAGGUGACUGGUACCC | 10052 | GGGUACCAGUCACCUGGAC |
| siRNA 3699 | 3699 | UCCAGGUGACUGGUACCCA | 10053 | UGGGUACCAGUCACCUGGA |
| siRNA 3700 | 3700 | CCAGGUGACUGGUACCCAC | 10054 | GUGGGUACCAGUCACCUGG |
| siRNA 3701 | 3701 | CAGGUGACUGGUACCCACU | 10055 | AGUGGGUACCAGUCACCUG |
| siRNA 3702 | 3702 | AGGUGACUGGUACCCACUC | 10056 | GAGUGGGUACCAGUCACCU |
| siRNA 3703 | 3703 | GGUGACUGGUACCCACUCG | 10057 | CGAGUGGGUACCAGUCACC |
| siRNA 3704 | 3704 | GUGACUGGUACCCACUCGA | 10058 | UCGAGUGGGUACCAGUCAC |
| siRNA 3705 | 3705 | UGACUGGUACCCACUCGAG | 10059 | CUCGAGUGGGUACCAGUCA |
| siRNA 3706 | 3706 | GACUGGUACCCACUCGAGU | 10060 | ACUCGAGUGGGUACCAGUC |
| siRNA 3707 | 3707 | ACUGGUACCCACUCGAGUU | 10061 | AACUCGAGUGGGUACCAGU |
| siRNA 3708 | 3708 | CUGGUACCCACUCGAGUUG | 10062 | CAACUCGAGUGGGUACCAG |
| siRNA 3709 | 3709 | UGGUACCCACUCGAGUUGU | 10063 | ACAACUCGAGUGGGUACCA |
| siRNA 3710 | 3710 | GGUACCCACUCGAGUUGUG | 10064 | CACAACUCGAGUGGGUACC |
| siRNA 3711 | 3711 | GUACCCACUCGAGUUGUGC | 10065 | GCACAACUCGAGUGGGUAC |
| siRNA 3712 | 3712 | UACCCACUCGAGUUGUGCC | 10066 | GGCACAACUCGAGUGGGUA |
| siRNA 3713 | 3713 | ACCCACUCGAGUUGUGCCG | 10067 | CGGCACAACUCGAGUGGGU |
| siRNA 3714 | 3714 | CCCACUCGAGUUGUGCCGU | 10068 | ACGGCACAACUCGAGUGGG |
| siRNA 3715 | 3715 | CCACUCGAGUUGUGCCGUG | 10069 | CACGGCACAACUCGAGUGG |
| siRNA 3716 | 3716 | CACUCGAGUUGUGCCGUGC | 10070 | GCACGGCACAACUCGAGUG |
| siRNA 3717 | 3717 | ACUCGAGUUGUGCCGUGCA | 10071 | UGCACGGCACAACUCGAGU |
| siRNA 3718 | 3718 | CUCGAGUUGUGCCGUGCAC | 10072 | GUGCACGGCACAACUCGAG |
| siRNA 3719 | 3719 | UCGAGUUGUGCCGUGCACA | 10073 | UGUGCACGGCACAACUCGA |
| siRNA 3720 | 3720 | CGAGUUGUGCCGUGCACAA | 10074 | UUGUGCACGGCACAACUCG |
| siRNA 3721 | 3721 | GAGUUGUGCCGUGCACAAC | 10075 | GUUGUGCACGGCACAACUC |
| siRNA 3722 | 3722 | AGUUGUGCCGUGCACAACC | 10076 | GGUUGUGCACGGCACAACU |
| siRNA 3723 | 3723 | GUUGUGCCGUGCACAACCU | 10077 | AGGUUGUGCACGGCACAAC |
| siRNA 3724 | 3724 | UUGUGCCGUGCACAACCUG | 10078 | CAGGUUGUGCACGGCACAA |
| siRNA 3725 | 3725 | UGUGCCGUGCACAACCUGU | 10079 | ACAGGUUGUGCACGGCACA |
| siRNA 3726 | 3726 | GUGCCGUGCACAACCUGUC | 10080 | GACAGGUUGUGCACGGCAC |
| siRNA 3727 | 3727 | UGCCGUGCACAACCUGUCC | 10081 | GGACAGGUUGUGCACGGCA |
| siRNA 3728 | 3728 | GCCGUGCACAACCUGUCCA | 10082 | UGGACAGGUUGUGCACGGC |
| siRNA 3729 | 3729 | CCGUGCACAACCUGUCCAG | 10083 | CUGGACAGGUUGUGCACGG |
| siRNA 3730 | 3730 | CGUGCACAACCUGUCCAGU | 10084 | ACUGGACAGGUUGUGCACG |
| siRNA 3731 | 3731 | GUGCACAACCUGUCCAGUA | 10085 | UACUGGACAGGUUGUGCAC |
| siRNA 3732 | 3732 | UGCACAACCUGUCCAGUAU | 10086 | AUACUGGACAGGUUGUGC |
| | | | | A |
| siRNA 3733 | 3733 | GCACAACCUGUCCAGUAUA | 10087 | UAUACUGGACAGGUUGUG |
| | | | | C |
| siRNA 3734 | 3734 | CACAACCUGUCCAGUAUAU | 10088 | AUAUACUGGACAGGUUGU |
| | | | | G |
| siRNA 3735 | 3735 | ACAACCUGUCCAGUAUAUG | 10089 | CAUAUACUGGACAGGUUG |
| | | | | U |
| siRNA 3736 | 3736 | CAACCUGUCCAGUAUAUGC | 10090 | GCAUAUACUGGACAGGUU |
| | | | | G |
| siRNA 3737 | 3737 | AACCUGUCCAGUAUAUGCA | 10091 | UGCAUAUACUGGACAGGU |
| | | | | U |
| siRNA 3738 | 3738 | ACCUGUCCAGUAUAUGCAU | 10092 | AUGCAUAUACUGGACAGG |
| | | | | U |
| siRNA 3739 | 3739 | CCUGUCCAGUAUAUGCAUG | 10093 | CAUGCAUAUACUGGACAGG |
| siRNA 3740 | 3740 | CUGUCCAGUAUAUGCAUGU | 10094 | ACAUGCAUAUACUGGACAG |
| siRNA 3741 | 3741 | UGUCCAGUAUAUGCAUGU | 10095 | CACAUGCAUAUACUGGACA |
| | | G | | |
| siRNA 3742 | 3742 | GUCCAGUAUAUGCAUGUG | 10096 | CCACAUGCAUAUACUGGAC |
| | | G | | |
| siRNA 3743 | 3743 | UCCAGUAUAUGCAUGUGG | 10097 | ACCACAUGCAUAUACUGGA |
| | | U | | |
| siRNA 3744 | 3744 | CCAGUAUAUGCAUGUGGU | 10098 | CACCACAUGCAUAUACUGG |
| | | G | | |
| siRNA 3745 | 3745 | CAGUAUAUGCAUGUGGUG | 10099 | CCACCACAUGCAUAUACUG |
| | | G | | |
| siRNA 3746 | 3746 | AGUAUAUGCAUGUGGUGG | 10100 | GCCACCACAUGCAUAUACU |
| | | C | | |
| siRNA 3747 | 3747 | GUAUAUGCAUGUGGUGGC | 10101 | GGCCACCACAUGCAUAUAC |
| | | C | | |
| siRNA 3748 | 3748 | UAUAUGCAUGUGGUGGCCC | 10102 | GGGCCACCACAUGCAUAUA |
| siRNA 3749 | 3749 | AUAUGCAUGUGGUGGCCCU | 10103 | AGGGCCACCACAUGCAUAU |
| siRNA 3750 | 3750 | UAUGCAUGUGGUGGCCCUA | 10104 | UAGGGCCACCACAUGCAUA |
| siRNA 3751 | 3751 | AUGCAUGUGGUGGCCCUAC | 10105 | GUAGGGCCACCACAUGCAU |
| siRNA 3752 | 3752 | UGCAUGUGGUGGCCCUACU | 10106 | AGUAGGGCCACCACAUGCA |
| siRNA 3753 | 3753 | GCAUGUGGUGGCCCUACUG | 10107 | CAGUAGGGCCACCACAUGC |
| siRNA 3754 | 3754 | CAUGUGGUGGCCCUACUGA | 10108 | UCAGUAGGGCCACCACAUG |
| siRNA 3755 | 3755 | AUGUGGUGGCCCUACUGAC | 10109 | GUCAGUAGGGCCACCACAU |
| siRNA 3756 | 3756 | UGUGGUGGCCCUACUGACU | 10110 | AGUCAGUAGGGCCACCACA |
| siRNA 3757 | 3757 | GUGGUGGCCCUACUGACUG | 10111 | CAGUCAGUAGGGCCACCAC |
| siRNA 3758 | 3758 | UGGUGGCCCUACUGACUGG | 10112 | CCAGUCAGUAGGGCCACCA |
| siRNA 3759 | 3759 | GGUGGCCCUACUGACUGGU | 10113 | ACCAGUCAGUAGGGCCACC |
| siRNA 3760 | 3760 | GUGGCCCUACUGACUGGUA | 10114 | UACCAGUCAGUAGGGCCAC |
| siRNA 3761 | 3761 | UGGCCCUACUGACUGGUAA | 10115 | UUACCAGUCAGUAGGGCCA |
| siRNA 3762 | 3762 | GGCCCUACUGACUGGUAAU | 10116 | AUUACCAGUCAGUAGGGCC |
| siRNA 3763 | 3763 | GCCCUACUGACUGGUAAUG | 10117 | CAUUACCAGUCAGUAGGGC |
| siRNA 3764 | 3764 | CCCUACUGACUGGUAAUGG | 10118 | CCAUUACCAGUCAGUAGGG |
| siRNA 3765 | 3765 | CCUACUGACUGGUAAUGGU | 10119 | ACCAUUACCAGUCAGUAGG |
| siRNA 3766 | 3766 | CUACUGACUGGUAAUGGU | 10120 | AACCAUUACCAGUCAGUAG |
| | | U | | |
| siRNA 3767 | 3767 | UACUGACUGGUAAUGGUU | 10121 | UAACCAUUACCAGUCAGUA |
| | | A | | |
| siRNA 3768 | 3768 | ACUGACUGGUAAUGGUUA | 10122 | CUAACCAUUACCAGUCAGU |
| | | G | | |
| siRNA 3769 | 3769 | CUGACUGGUAAUGGUUAG | 10123 | UCUAACCAUUACCAGUCAG |
| | | A | | |
| siRNA 3770 | 3770 | UGACUGGUAAUGGUUAGA | 10124 | CUCUAACCAUUACCAGUCA |
| | | G | | |
| siRNA 3771 | 3771 | GACUGGUAAUGGUUAGAG | 10125 | CCUCUAACCAUUACCAGUC |
| | | G | | |
| siRNA 3772 | 3772 | ACUGGUAAUGGUUAGAGG | 10126 | GCCUCUAACCAUUACCAGU |
| | | C | | |
| siRNA 3773 | 3773 | CUGGUAAUGGUUAGAGGC | 10127 | UGCCUCUAACCAUUACCAG |
| | | A | | |
| siRNA 3774 | 3774 | UGGUAAUGGUUAGAGGCA | 10128 | AUGCCUCUAACCAUUACCA |
| | | U | | |
| siRNA 3775 | 3775 | GGUAAUGGUUAGAGGCAU | 10129 | AAUGCCUCUAACCAUUACC |
| | | U | | |
| siRNA 3776 | 3776 | GUAAUGGUUAGAGGCAUU | 10130 | AAAUGCCUCUAACCAUUAC |
| | | U | | |
| siRNA 3777 | 3777 | UAAUGGUUAGAGGCAUUU | 10131 | UAAAUGCCUCUAACCAUUA |
| | | A | | |
| siRNA 3778 | 3778 | AAUGGUUAGAGGCAUUUA | 10132 | AUAAAUGCCUCUAACCAUU |
| | | U | | |
| siRNA 3779 | 3779 | AUGGUUAGAGGCAUUUAU | 10133 | CAUAAAUGCCUCUAACCAU |
| | | G | | |
| siRNA 3780 | 3780 | UGGUUAGAGGCAUUUAUG | 10134 | CCAUAAAUGCCUCUAACCA |
| | | G | | |
| siRNA 3781 | 3781 | GGUUAGAGGCAUUUAUGG | 10135 | UCCAUAAAUGCCUCUAACC |
| | | A | | |
| siRNA 3782 | 3782 | GUUAGAGGCAUUUAUGGA | 10136 | AUCCAUAAAUGCCUCUAAC |
| | | U | | |
| siRNA 3783 | 3783 | UUAGAGGCAUUUAUGGAU | 10137 | AAUCCAUAAAUGCCUCUAA |
| | | U | | |
| siRNA 3784 | 3784 | UAGAGGCAUUUAUGGAUU | 10138 | AAAUCCAUAAAUGCCUCUA |
| | | U | | |
| siRNA 3785 | 3785 | AGAGGCAUUUAUGGAUUU | 10139 | AAAAUCCAUAAAUGCCUCU |
| | | U | | |
| siRNA 3786 | 3786 | GAGGCAUUUAUGGAUUUU | 10140 | AAAAAUCCAUAAAUGCCUC |
| | | U | | |
| siRNA 3787 | 3787 | AGGCAUUUAUGGAUUUUU | 10141 | UAAAAAUCCAUAAAUGCCU |
| | | A | | |
| siRNA 3788 | 3788 | GGCAUUUAUGGAUUUUUA | 10142 | CUAAAAAUCCAUAAAUGCC |
| | | G | | |
| siRNA 3789 | 3789 | GCAUUUAUGGAUUUUUAG | 10143 | GCUAAAAAUCCAUAAAUGC |
| | | C | | |
| siRNA 3790 | 3790 | CAUUUAUGGAUUUUUAGC | 10144 | AGCUAAAAAUCCAUAAAU |
| | | U | | G |
| siRNA 3791 | 3791 | AUUUAUGGAUUUUUAGCU | 10145 | AAGCUAAAAAUCCAUAAA |
| | | U | | U |
| siRNA 3792 | 3792 | UUUAUGGAUUUUUAGCUU | 10146 | AAAGCUAAAAAUCCAUAA |
| | | U | | A |
| siRNA 3793 | 3793 | UUAUGGAUUUUUAGCUUU | 10147 | CAAAGCUAAAAAUCCAUAA |
| | | G | | |
| siRNA 3794 | 3794 | UAUGGAUUUUUAGCUUUG | 10148 | UCAAAGCUAAAAAUCCAUA |
| | | A | | |
| siRNA 3795 | 3795 | AUGGAUUUUUAGCUUUGA | 10149 | CUCAAAGCUAAAAAUCCAU |
| | | G | | |
| siRNA 3796 | 3796 | UGGAUUUUUAGCUUUGAG | 10150 | CCUCAAAGCUAAAAAUCCA |
| | | G | | |
| siRNA 3797 | 3797 | GGAUUUUUAGCUUUGAGG | 10151 | UCCUCAAAGCUAAAAAUCC |
| | | A | | |
| siRNA 3798 | 3798 | GAUUUUUAGCUUUGAGGA | 10152 | UUCCUCAAAGCUAAAAAUC |
| | | A | | |
| siRNA 3799 | 3799 | AUUUUUAGCUUUGAGGAA | 10153 | UUUCCUCAAAGCUAAAAAU |
| | | A | | |
| siRNA 3800 | 3800 | UUUUUAGCUUUGAGGAAA | 10154 | UUUUCCUCAAAGCUAAAAA |
| | | A | | |
| siRNA 3801 | 3801 | UUUUAGCUUUGAGGAAAA | 10155 | UUUUUCCUCAAAGCUAAAA |
| | | A | | |
| siRNA 3802 | 3802 | UUUAGCUUUGAGGAAAAA | 10156 | GUUUUUCCUCAAAGCUAAA |
| | | C | | |
| siRNA 3803 | 3803 | UUAGCUUUGAGGAAAAAC | 10157 | GGUUUUUCCUCAAAGCUAA |
| | | C | | |
| siRNA 3804 | 3804 | UAGCUUUGAGGAAAAACC | 10158 | UGGUUUUUCCUCAAAGCUA |
| | | A | | |
| siRNA 3805 | 3805 | AGCUUUGAGGAAAAACCA | 10159 | AUGGUUUUUCCUCAAAGCU |
| | | U | | |
| siRNA 3806 | 3806 | GCUUUGAGGAAAAACCAU | 10160 | CAUGGUUUUUCCUCAAAGC |
| | | G | | |
| siRNA 3807 | 3807 | CUUUGAGGAAAAACCAUG | 10161 | UCAUGGUUUUUCCUCAAAG |
| | | A | | |
| siRNA 3808 | 3808 | UUUGAGGAAAAACCAUGA | 10162 | GUCAUGGUUUUUCCUCAAA |
| | | C | | |
| siRNA 3809 | 3809 | UUGAGGAAAAACCAUGAC | 10163 | AGUCAUGGUUUUUCCUCAA |
| | | U | | |
| siRNA 3810 | 3810 | UGAGGAAAAACCAUGACU | 10164 | AAGUCAUGGUUUUUCCUCA |
| | | U | | |
| siRNA 3811 | 3811 | GAGGAAAAACCAUGACUU | 10165 | AAAGUCAUGGUUUUUCCUC |
| | | U | | |
| siRNA 3812 | 3812 | AGGAAAAACCAUGACUUU | 10166 | AAAAGUCAUGGUUUUUCC |
| | | U | | U |
| siRNA 3813 | 3813 | GGAAAAACCAUGACUUUU | 10167 | UAAAAGUCAUGGUUUUUC |
| | | A | | C |
| siRNA 3814 | 3814 | GAAAAACCAUGACUUUUA | 10168 | UUAAAAGUCAUGGUUUUU |
| | | A | | C |
| siRNA 3815 | 3815 | AAAAACCAUGACUUUUAAC | 10169 | GUUAAAAGUCAUGGUUUU |
| | | | | U |
| siRNA 3816 | 3816 | AAAACCAUGACUUUUAACA | 10170 | UGUUAAAAGUCAUGGUUU |
| | | | | U |
| siRNA 3817 | 3817 | AAACCAUGACUUUUAACAA | 10171 | UUGUUAAAAGUCAUGGUU |
| | | | | U |
| siRNA 3818 | 3818 | AACCAUGACUUUUAACAAA | 10172 | UUUGUUAAAAGUCAUGGU |
| | | | | U |
| siRNA 3819 | 3819 | ACCAUGACUUUUAACAAAU | 10173 | AUUUGUUAAAAGUCAUGG |
| | | | | U |
| siRNA 3820 | 3820 | CCAUGACUUUUAACAAAUU | 10174 | AAUUUGUUAAAAGUCAUG |
| | | | | G |
| siRNA 3821 | 3821 | CAUGACUUUUAACAAAUU | 10175 | AAAUUUGUUAAAAGUCAU |
| | | U | | G |
| siRNA 3822 | 3822 | AUGACUUUUAACAAAUUU | 10176 | AAAAUUUGUUAAAAGUCA |
| | | U | | U |
| siRNA 3823 | 3823 | UGACUUUUAACAAAUUUU | 10177 | AAAAAUUUGUUAAAAGUC |
| | | U | | A |
| siRNA 3824 | 3824 | GACUUUUAACAAAUUUUU | 10178 | UAAAAAUUUGUUAAAAGU |
| | | A | | C |
| siRNA 3825 | 3825 | ACUUUUAACAAAUUUUUA | 10179 | AUAAAAAUUUGUUAAAAG |
| | | U | | U |
| siRNA 3826 | 3826 | CUUUUAACAAAUUUUUAU | 10180 | CAUAAAAAUUUGUUAAAA |
| | | G | | G |
| siRNA 3827 | 3827 | UUUUAACAAAUUUUUAUG | 10181 | CCAUAAAAAUUUGUUAAA |
| | | G | | A |
| siRNA 3828 | 3828 | UUUAACAAAUUUUUAUGG | 10182 | CCCAUAAAAAUUUGUUAA |
| | | G | | A |
| siRNA 3829 | 3829 | UUAACAAAUUUUUAUGGG | 10183 | ACCCAUAAAAAUUUGUUA |
| | | U | | A |
| siRNA 3830 | 3830 | UAACAAAUUUUUAUGGGU | 10184 | AACCCAUAAAAAUUUGUU |
| | | U | | A |
| siRNA 3831 | 3831 | AACAAAUUUUUAUGGGUU | 10185 | UAACCCAUAAAAAUUUGU |
| | | A | | U |
| siRNA 3832 | 3832 | ACAAAUUUUUAUGGGUUA | 10186 | AUAACCCAUAAAAAUUUG |
| | | U | | U |
| siRNA 3833 | 3833 | CAAAUUUUUAUGGGUUAU | 10187 | UAUAACCCAUAAAAAUUU |
| | | A | | G |
| siRNA 3834 | 3834 | AAAUUUUUAUGGGUUAUA | 10188 | AUAUAACCCAUAAAAAUU |
| | | U | | U |
| siRNA 3835 | 3835 | AAUUUUUAUGGGUUAUAU | 10189 | CAUAUAACCCAUAAAAAUU |
| | | G | | |
| siRNA 3836 | 3836 | AUUUUUAUGGGUUAUAUG | 10190 | GCAUAUAACCCAUAAAAAU |
| | | C | | |
| siRNA 3837 | 3837 | UUUUUAUGGGUUAUAUGC | 10191 | GGCAUAUAACCCAUAAAAA |
| | | C | | |
| siRNA 3838 | 3838 | UUUUAUGGGUUAUAUGCC | 10192 | AGGCAUAUAACCCAUAAAA |
| | | U | | |
| siRNA 3839 | 3839 | UUUAUGGGUUAUAUGCCU | 10193 | UAGGCAUAUAACCCAUAAA |
| | | A | | |
| siRNA 3840 | 3840 | UUAUGGGUUAUAUGCCUA | 10194 | UUAGGCAUAUAACCCAUAA |
| | | A | | |
| siRNA 3841 | 3841 | UAUGGGUUAUAUGCCUAA | 10195 | UUUAGGCAUAUAACCCAUA |
| | | A | | |
| siRNA 3842 | 3842 | AUGGGUUAUAUGCCUAAA | 10196 | GUUUAGGCAUAUAACCCAU |
| | | C | | |
| siRNA 3843 | 3843 | UGGGUUAUAUGCCUAAACC | 10197 | GGUUUAGGCAUAUAACCCA |
| siRNA 3844 | 3844 | GGGUUAUAUGCCUAAACCC | 10198 | GGGUUUAGGCAUAUAACCC |
| siRNA 3845 | 3845 | GGUUAUAUGCCUAAACCCU | 10199 | AGGGUUUAGGCAUAUAAC |
| | | | | C |
| siRNA 3846 | 3846 | GUUAUAUGCCUAAACCCUU | 10200 | AAGGGUUUAGGCAUAUAA |
| | | | | C |
| siRNA 3847 | 3847 | UUAUAUGCCUAAACCCUUA | 10201 | UAAGGGUUUAGGCAUAUA |
| | | | | A |
| siRNA 3848 | 3848 | UAUAUGCCUAAACCCUUAU | 10202 | AUAAGGGUUUAGGCAUAU |
| | | | | A |
| siRNA 3849 | 3849 | AUAUGCCUAAACCCUUAUG | 10203 | CAUAAGGGUUUAGGCAUA |
| | | | | U |
| siRNA 3850 | 3850 | UAUGCCUAAACCCUUAUGC | 10204 | GCAUAAGGGUUUAGGCAU |
| | | | | A |
| siRNA 3851 | 3851 | AUGCCUAAACCCUUAUGCC | 10205 | GGCAUAAGGGUUUAGGCA |
| | | | | U |
| siRNA 3852 | 3852 | UGCCUAAACCCUUAUGCCA | 10206 | UGGCAUAAGGGUUUAGGC |
| | | | | A |
| siRNA 3853 | 3853 | GCCUAAACCCUUAUGCCAC | 10207 | GUGGCAUAAGGGUUUAGG |
| | | | | C |
| siRNA 3854 | 3854 | CCUAAACCCUUAUGCCACA | 10208 | UGUGGCAUAAGGGUUUAG |
| | | | | G |
| siRNA 3855 | 3855 | CUAAACCCUUAUGCCACAU | 10209 | AUGUGGCAUAAGGGUUUA |
| | | | | G |
| siRNA 3856 | 3856 | UAAACCCUUAUGCCACAUA | 10210 | UAUGUGGCAUAAGGGUUU |
| | | | | A |
| siRNA 3857 | 3857 | AAACCCUUAUGCCACAUAG | 10211 | CUAUGUGGCAUAAGGGUU |
| | | | | U |
| siRNA 3858 | 3858 | AACCCUUAUGCCACAUAGU | 10212 | ACUAUGUGGCAUAAGGGU |
| | | | | U |
| siRNA 3859 | 3859 | ACCCUUAUGCCACAUAGUG | 10213 | CACUAUGUGGCAUAAGGG |
| | | | | U |
| siRNA 3860 | 3860 | CCCUUAUGCCACAUAGUGG | 10214 | CCACUAUGUGGCAUAAGGG |
| siRNA 3861 | 3861 | CCUUAUGCCACAUAGUGGU | 10215 | ACCACUAUGUGGCAUAAGG |
| siRNA 3862 | 3862 | CUUAUGCCACAUAGUGGUA | 10216 | UACCACUAUGUGGCAUAAG |
| siRNA 3863 | 3863 | UUAUGCCACAUAGUGGUA | 10217 | UUACCACUAUGUGGCAUAA |
| | | A | | |
| siRNA 3864 | 3864 | UAUGCCACAUAGUGGUAA | 10218 | UUUACCACUAUGUGGCAUA |
| | | A | | |
| siRNA 3865 | 3865 | AUGCCACAUAGUGGUAAA | 10219 | AUUUACCACUAUGUGGCAU |
| | | U | | |
| siRNA 3866 | 3866 | UGCCACAUAGUGGUAAAU | 10220 | UAUUUACCACUAUGUGGCA |
| | | A | | |
| siRNA 3867 | 3867 | GCCACAUAGUGGUAAAUA | 10221 | UUAUUUACCACUAUGUGGC |
| | | A | | |
| siRNA 3868 | 3868 | CCACAUAGUGGUAAAUAA | 10222 | AUUAUUUACCACUAUGUG |
| | | U | | G |
| siRNA 3869 | 3869 | CACAUAGUGGUAAAUAAU | 10223 | AAUUAUUUACCACUAUGU |
| | | U | | G |
| siRNA 3870 | 3870 | ACAUAGUGGUAAAUAAUU | 10224 | UAAUUAUUUACCACUAUG |
| | | A | | U |
| siRNA 3871 | 3871 | CAUAGUGGUAAAUAAUUA | 10225 | AUAAUUAUUUACCACUAU |
| | | U | | G |
| siRNA 3872 | 3872 | AUAGUGGUAAAUAAUUAU | 10226 | CAUAAUUAUUUACCACUAU |
| | | G | | |
| siRNA 3873 | 3873 | UAGUGGUAAAUAAUUAUG | 10227 | UCAUAAUUAUUUACCACUA |
| | | A | | |
| siRNA 3874 | 3874 | AGUGGUAAAUAAUUAUGA | 10228 | UUCAUAAUUAUUUACCACU |
| | | A | | |
| siRNA 3875 | 3875 | GUGGUAAAUAAUUAUGAA | 10229 | UUUCAUAAUUAUUUACCAC |
| | | A | | |
| siRNA 3876 | 3876 | UGGUAAAUAAUUAUGAAA | 10230 | UUUUCAUAAUUAUUUACC |
| | | A | | A |
| siRNA 3877 | 3877 | GGUAAAUAAUUAUGAAAA | 10231 | UUUUUCAUAAUUAUUUAC |
| | | A | | C |
| siRNA 3878 | 3878 | GUAAAUAAUUAUGAAAAA | 10232 | AUUUUUCAUAAUUAUUUA |
| | | U | | C |
| siRNA 3879 | 3879 | UAAAUAAUUAUGAAAAAU | 10233 | CAUUUUUCAUAAUUAUUU |
| | | G | | A |
| siRNA 3880 | 3880 | AAAUAAUUAUGAAAAAUG | 10234 | CCAUUUUUCAUAAUUAUU |
| | | G | | U |
| siRNA 3881 | 3881 | AAUAAUUAUGAAAAAUGG | 10235 | ACCAUUUUUCAUAAUUAU |
| | | U | | U |
| siRNA 3882 | 3882 | AUAAUUAUGAAAAAUGGU | 10236 | GACCAUUUUUCAUAAUUA |
| | | C | | U |
| siRNA 3883 | 3883 | UAAUUAUGAAAAAUGGUC | 10237 | AGACCAUUUUUCAUAAUU |
| | | U | | A |
| siRNA 3884 | 3884 | AAUUAUGAAAAAUGGUCU | 10238 | CAGACCAUUUUUCAUAAUU |
| | | G | | |
| siRNA 3885 | 3885 | AUUAUGAAAAAUGGUCUG | 10239 | ACAGACCAUUUUUCAUAAU |
| | | U | | |
| siRNA 3886 | 3886 | UUAUGAAAAAUGGUCUGU | 10240 | AACAGACCAUUUUUCAUAA |
| | | U | | |
| siRNA 3887 | 3887 | UAUGAAAAAUGGUCUGUU | 10241 | GAACAGACCAUUUUUCAUA |
| | | C | | |
| siRNA 3888 | 3888 | AUGAAAAAUGGUCUGUUC | 10242 | UGAACAGACCAUUUUUCAU |
| | | A | | |
| siRNA 3889 | 3889 | UGAAAAAUGGUCUGUUCA | 10243 | AUGAACAGACCAUUUUUCA |
| | | U | | |
| siRNA 3890 | 3890 | GAAAAAUGGUCUGUUCAU | 10244 | UAUGAACAGACCAUUUUUC |
| | | A | | |
| siRNA 3891 | 3891 | AAAAAUGGUCUGUUCAUA | 10245 | UUAUGAACAGACCAUUUU |
| | | A | | U |
| siRNA 3892 | 3892 | AAAAUGGUCUGUUCAUAA | 10246 | AUUAUGAACAGACCAUUU |
| | | U | | U |
| siRNA 3893 | 3893 | AAAUGGUCUGUUCAUAAU | 10247 | AAUUAUGAACAGACCAUU |
| | | U | | U |
| siRNA 3894 | 3894 | AAUGGUCUGUUCAUAAUU | 10248 | CAAUUAUGAACAGACCAUU |
| | | G | | |
| siRNA 3895 | 3895 | AUGGUCUGUUCAUAAUUG | 10249 | CCAAUUAUGAACAGACCAU |
| | | G | | |
| siRNA 3896 | 3896 | UGGUCUGUUCAUAAUUGG | 10250 | ACCAAUUAUGAACAGACCA |
| | | U | | |
| siRNA 3897 | 3897 | GGUCUGUUCAUAAUUGGU | 10251 | UACCAAUUAUGAACAGACC |
| | | A | | |
| siRNA 3898 | 3898 | GUCUGUUCAUAAUUGGUA | 10252 | CUACCAAUUAUGAACAGAC |
| | | G | | |
| siRNA 3899 | 3899 | UCUGUUCAUAAUUGGUAG | 10253 | CCUACCAAUUAUGAACAGA |
| | | G | | |
| siRNA 3900 | 3900 | CUGUUCAUAAUUGGUAGG | 10254 | ACCUACCAAUUAUGAACAG |
| | | U | | |
| siRNA 3901 | 3901 | UGUUCAUAAUUGGUAGGU | 10255 | CACCUACCAAUUAUGAACA |
| | | G | | |
| siRNA 3902 | 3902 | GUUCAUAAUUGGUAGGUG | 10256 | GCACCUACCAAUUAUGAAC |
| | | C | | |
| siRNA 3903 | 3903 | UUCAUAAUUGGUAGGUGC | 10257 | GGCACCUACCAAUUAUGAA |
| | | C | | |
| siRNA 3904 | 3904 | UCAUAAUUGGUAGGUGCC | 10258 | AGGCACCUACCAAUUAUGA |
| | | U | | |
| siRNA 3905 | 3905 | CAUAAUUGGUAGGUGCCU | 10259 | AAGGCACCUACCAAUUAUG |
| | | U | | |
| siRNA 3906 | 3906 | AUAAUUGGUAGGUGCCUU | 10260 | AAAGGCACCUACCAAUUAU |
| | | U | | |
| siRNA 3907 | 3907 | UAAUUGGUAGGUGCCUUU | 10261 | AAAAGGCACCUACCAAUUA |
| | | U | | |
| siRNA 3908 | 3908 | AAUUGGUAGGUGCCUUUU | 10262 | CAAAAGGCACCUACCAAUU |
| | | G | | |
| siRNA 3909 | 3909 | AUUGGUAGGUGCCUUUUG | 10263 | ACAAAAGGCACCUACCAAU |
| | | U | | |
| siRNA 3910 | 3910 | UUGGUAGGUGCCUUUUGU | 10264 | CACAAAAGGCACCUACCAA |
| | | G | | |
| siRNA 3911 | 3911 | UGGUAGGUGCCUUUUGUG | 10265 | UCACAAAAGGCACCUACCA |
| | | A | | |
| siRNA 3912 | 3912 | GGUAGGUGCCUUUUGUGA | 10266 | CUCACAAAAGGCACCUACC |
| | | G | | |
| siRNA 3913 | 3913 | GUAGGUGCCUUUUGUGAG | 10267 | GCUCACAAAAGGCACCUAC |
| | | C | | |
| siRNA 3914 | 3914 | UAGGUGCCUUUUGUGAGC | 10268 | UGCUCACAAAAGGCACCUA |
| | | A | | |
| siRNA 3915 | 3915 | AGGUGCCUUUUGUGAGCA | 10269 | CUGCUCACAAAAGGCACCU |
| | | G | | |
| siRNA 3916 | 3916 | GGUGCCUUUUGUGAGCAG | 10270 | CCUGCUCACAAAAGGCACC |
| | | G | | |
| siRNA 3917 | 3917 | GUGCCUUUUGUGAGCAGG | 10271 | CCCUGCUCACAAAAGGCAC |
| | | G | | |
| siRNA 3918 | 3918 | UGCCUUUUGUGAGCAGGG | 10272 | UCCCUGCUCACAAAAGGCA |
| | | A | | |
| siRNA 3919 | 3919 | GCCUUUUGUGAGCAGGGA | 10273 | CUCCCUGCUCACAAAAGGC |
| | | G | | |
| siRNA 3920 | 3920 | CCUUUUGUGAGCAGGGAGC | 10274 | GCUCCCUGCUCACAAAAGG |
| siRNA 3921 | 3921 | CUUUUGUGAGCAGGGAGC | 10275 | UGCUCCCUGCUCACAAAAG |
| | | A | | |
| siRNA 3922 | 3922 | UUUUGUGAGCAGGGAGCA | 10276 | AUGCUCCCUGCUCACAAAA |
| | | U | | |
| siRNA 3923 | 3923 | UUUGUGAGCAGGGAGCAU | 10277 | UAUGCUCCCUGCUCACAAA |
| | | A | | |
| siRNA 3924 | 3924 | UUGUGAGCAGGGAGCAUA | 10278 | UUAUGCUCCCUGCUCACAA |
| | | A | | |
| siRNA 3925 | 3925 | UGUGAGCAGGGAGCAUAA | 10279 | AUUAUGCUCCCUGCUCACA |
| | | U | | |
| siRNA 3926 | 3926 | GUGAGCAGGGAGCAUAAU | 10280 | AAUUAUGCUCCCUGCUCAC |
| | | U | | |
| siRNA 3927 | 3927 | UGAGCAGGGAGCAUAAUU | 10281 | UAAUUAUGCUCCCUGCUCA |
| | | A | | |
| siRNA 3928 | 3928 | GAGCAGGGAGCAUAAUUA | 10282 | AUAAUUAUGCUCCCUGCUC |
| | | U | | |
| siRNA 3929 | 3929 | AGCAGGGAGCAUAAUUAU | 10283 | AAUAAUUAUGCUCCCUGCU |
| | | U | | |
| siRNA 3930 | 3930 | GCAGGGAGCAUAAUUAUU | 10284 | CAAUAAUUAUGCUCCCUGC |
| | | G | | |
| siRNA 3931 | 3931 | CAGGGAGCAUAAUUAUUG | 10285 | CCAAUAAUUAUGCUCCCUG |
| | | G | | |
| siRNA 3932 | 3932 | AGGGAGCAUAAUUAUUGG | 10286 | ACCAAUAAUUAUGCUCCCU |
| | | U | | |
| siRNA 3933 | 3933 | GGGAGCAUAAUUAUUGGU | 10287 | AACCAAUAAUUAUGCUCCC |
| | | U | | |
| siRNA 3934 | 3934 | GGAGCAUAAUUAUUGGUU | 10288 | AAACCAAUAAUUAUGCUCC |
| | | U | | |
| siRNA 3935 | 3935 | GAGCAUAAUUAUUGGUUU | 10289 | UAAACCAAUAAUUAUGCUC |
| | | A | | |
| siRNA 3936 | 3936 | AGCAUAAUUAUUGGUUUA | 10290 | AUAAACCAAUAAUUAUGC |
| | | U | | U |
| siRNA 3937 | 3937 | GCAUAAUUAUUGGUUUAU | 10291 | AAUAAACCAAUAAUUAUG |
| | | U | | C |
| siRNA 3938 | 3938 | CAUAAUUAUUGGUUUAUU | 10292 | UAAUAAACCAAUAAUUAU |
| | | A | | G |
| siRNA 3939 | 3939 | AUAAUUAUUGGUUUAUUA | 10293 | AUAAUAAACCAAUAAUUA |
| | | U | | U |
| siRNA 3940 | 3940 | UAAUUAUUGGUUUAUUAU | 10294 | CAUAAUAAACCAAUAAUU |
| | | G | | A |
| siRNA 3941 | 3941 | AAUUAUUGGUUUAUUAUG | 10295 | CCAUAAUAAACCAAUAAUU |
| | | G | | |
| siRNA 3942 | 3942 | AUUAUUGGUUUAUUAUGG | 10296 | ACCAUAAUAAACCAAUAAU |
| | | U | | |
| siRNA 3943 | 3943 | UUAUUGGUUUAUUAUGGU | 10297 | UACCAUAAUAAACCAAUAA |
| | | A | | |
| siRNA 3944 | 3944 | UAUUGGUUUAUUAUGGUA | 10298 | UUACCAUAAUAAACCAAUA |
| | | A | | |
| siRNA 3945 | 3945 | AUUGGUUUAUUAUGGUAA | 10299 | AUUACCAUAAUAAACCAAU |
| | | U | | |
| siRNA 3946 | 3946 | UUGGUUUAUUAUGGUAAU | 10300 | AAUUACCAUAAUAAACCAA |
| | | U | | |
| siRNA 3947 | 3947 | UGGUUUAUUAUGGUAAUU | 10301 | UAAUUACCAUAAUAAACCA |
| | | A | | |
| siRNA 3948 | 3948 | GGUUUAUUAUGGUAAUUA | 10302 | AUAAUUACCAUAAUAAACC |
| | | U | | |
| siRNA 3949 | 3949 | GUUUAUUAUGGUAAUUAU | 10303 | CAUAAUUACCAUAAUAAAC |
| | | G | | |
| siRNA 3950 | 3950 | UUUAUUAUGGUAAUUAUG | 10304 | CCAUAAUUACCAUAAUAAA |
| | | G | | |
| siRNA 3951 | 3951 | UUAUUAUGGUAAUUAUGG | 10305 | ACCAUAAUUACCAUAAUAA |
| | | U | | |
| siRNA 3952 | 3952 | UAUUAUGGUAAUUAUGGU | 10306 | CACCAUAAUUACCAUAAUA |
| | | G | | |
| siRNA 3953 | 3953 | AUUAUGGUAAUUAUGGUG | 10307 | UCACCAUAAUUACCAUAAU |
| | | A | | |
| siRNA 3954 | 3954 | UUAUGGUAAUUAUGGUGA | 10308 | AUCACCAUAAUUACCAUAA |
| | | U | | |
| siRNA 3955 | 3955 | UAUGGUAAUUAUGGUGAU | 10309 | AAUCACCAUAAUUACCAUA |
| | | U | | |
| siRNA 3956 | 3956 | AUGGUAAUUAUGGUGAUU | 10310 | AAAUCACCAUAAUUACCAU |
| | | U | | |
| siRNA 3957 | 3957 | UGGUAAUUAUGGUGAUUU | 10311 | AAAAUCACCAUAAUUACCA |
| | | U | | |
| siRNA 3958 | 3958 | GGUAAUUAUGGUGAUUUU | 10312 | AAAAAUCACCAUAAUUACC |
| | | U | | |
| siRNA 3959 | 3959 | GUAAUUAUGGUGAUUUUU | 10313 | AAAAAAUCACCAUAAUUAC |
| | | U | | |
| siRNA 3960 | 3960 | UAAUUAUGGUGAUUUUUU | 10314 | UAAAAAAUCACCAUAAUU |
| | | A | | A |
| siRNA 3961 | 3961 | AAUUAUGGUGAUUUUUUA | 10315 | UUAAAAAAUCACCAUAAU |
| | | A | | U |
| siRNA 3962 | 3962 | AUUAUGGUGAUUUUUUAA | 10316 | UUUAAAAAAUCACCAUAA |
| | | A | | U |
| siRNA 3963 | 3963 | UUAUGGUGAUUUUUUAAA | 10317 | AUUUAAAAAAUCACCAUA |
| | | U | | A |
| siRNA 3964 | 3964 | UAUGGUGAUUUUUUAAAU | 10318 | UAUUUAAAAAAUCACCAU |
| | | A | | A |
| siRNA 3965 | 3965 | AUGGUGAUUUUUUAAAUA | 10319 | AUAUUUAAAAAAUCACCA |
| | | U | | U |
| siRNA 3966 | 3966 | UGGUGAUUUUUUAAAUAU | 10320 | GAUAUUUAAAAAAUCACC |
| | | C | | A |
| siRNA 3967 | 3967 | GGUGAUUUUUUAAAUAUC | 10321 | UGAUAUUUAAAAAAUCAC |
| | | A | | C |
| siRNA 3968 | 3968 | GUGAUUUUUUAAAUAUCA | 10322 | AUGAUAUUUAAAAAAUCA |
| | | U | | C |
| siRNA 3969 | 3969 | UGAUUUUUUAAAUAUCAU | 10323 | CAUGAUAUUUAAAAAAUC |
| | | G | | A |
| siRNA 3970 | 3970 | GAUUUUUUAAAUAUCAUG | 10324 | ACAUGAUAUUUAAAAAAU |
| | | U | | C |
| siRNA 3971 | 3971 | AUUUUUUAAAUAUCAUGU | 10325 | UACAUGAUAUUUAAAAAA |
| | | A | | U |
| siRNA 3972 | 3972 | UUUUUUAAAUAUCAUGUA | 10326 | UUACAUGAUAUUUAAAAA |
| | | A | | A |
| siRNA 3973 | 3973 | UUUUUAAAUAUCAUGUAA | 10327 | AUUACAUGAUAUUUAAAA |
| | | U | | A |
| siRNA 3974 | 3974 | UUUUAAAUAUCAUGUAAU | 10328 | CAUUACAUGAUAUUUAAA |
| | | G | | A |
| siRNA 3975 | 3975 | UUUAAAUAUCAUGUAAUG | 10329 | ACAUUACAUGAUAUUUAA |
| | | U | | A |
| siRNA 3976 | 3976 | UUAAAUAUCAUGUAAUGU | 10330 | AACAUUACAUGAUAUUUA |
| | | U | | A |
| siRNA 3977 | 3977 | UAAAUAUCAUGUAAUGUU | 10331 | UAACAUUACAUGAUAUUU |
| | | A | | A |
| siRNA 3978 | 3978 | AAAUAUCAUGUAAUGUUA | 10332 | UUAACAUUACAUGAUAUU |
| | | A | | U |
| siRNA 3979 | 3979 | AAUAUCAUGUAAUGUUAA | 10333 | UUUAACAUUACAUGAUAU |
| | | A | | U |
| siRNA 3980 | 3980 | AUAUCAUGUAAUGUUAAA | 10334 | UUUUAACAUUACAUGAUA |
| | | A | | U |
| siRNA 3981 | 3981 | UAUCAUGUAAUGUUAAAA | 10335 | GUUUUAACAUUACAUGAU |
| | | C | | A |
| siRNA 3982 | 3982 | AUCAUGUAAUGUUAAAAC | 10336 | CGUUUUAACAUUACAUGA |
| | | G | | U |
| siRNA 3983 | 3983 | UCAUGUAAUGUUAAAACG | 10337 | ACGUUUUAACAUUACAUG |
| | | U | | A |
| siRNA 3984 | 3984 | CAUGUAAUGUUAAAACGU | 10338 | AACGUUUUAACAUUACAU |
| | | U | | G |
| siRNA 3985 | 3985 | AUGUAAUGUUAAAACGUU | 10339 | AAACGUUUUAACAUUACA |
| | | U | | U |
| siRNA 3986 | 3986 | UGUAAUGUUAAAACGUUU | 10340 | AAAACGUUUUAACAUUAC |
| | | U | | A |
| siRNA 3987 | 3987 | GUAAUGUUAAAACGUUUU | 10341 | GAAAACGUUUUAACAUUA |
| | | C | | C |
| siRNA 3988 | 3988 | UAAUGUUAAAACGUUUUC | 10342 | AGAAAACGUUUUAACAUU |
| | | U | | A |
| siRNA 3989 | 3989 | AAUGUUAAAACGUUUUCU | 10343 | UAGAAAACGUUUUAACAU |
| | | A | | U |
| siRNA 3990 | 3990 | AUGUUAAAACGUUUUCUA | 10344 | UUAGAAAACGUUUUAACA |
| | | A | | U |
| siRNA 3991 | 3991 | UGUUAAAACGUUUUCUAA | 10345 | GUUAGAAAACGUUUUAAC |
| | | C | | A |
| siRNA 3992 | 3992 | GUUAAAACGUUUUCUAAC | 10346 | UGUUAGAAAACGUUUUAA |
| | | A | | C |
| siRNA 3993 | 3993 | UUAAAACGUUUUCUAACA | 10347 | CUGUUAGAAAACGUUUUA |
| | | G | | A |
| siRNA 3994 | 3994 | UAAAACGUUUUCUAACAG | 10348 | ACUGUUAGAAAACGUUUU |
| | | U | | A |
| siRNA 3995 | 3995 | AAAACGUUUUCUAACAGU | 10349 | AACUGUUAGAAAACGUUU |
| | | U | | U |
| siRNA 3996 | 3996 | AAACGUUUUCUAACAGUU | 10350 | AAACUGUUAGAAAACGUU |
| | | U | | U |
| siRNA 3997 | 3997 | AACGUUUUCUAACAGUUU | 10351 | UAAACUGUUAGAAAACGU |
| | | A | | U |
| siRNA 3998 | 3998 | ACGUUUUCUAACAGUUUAC | 10352 | GUAAACUGUUAGAAAACG |
| | | | | U |
| siRNA 3999 | 3999 | CGUUUUCUAACAGUUUACU | 10353 | AGUAAACUGUUAGAAAAC |
| | | | | G |
| siRNA 4000 | 4000 | GUUUUCUAACAGUUUACU | 10354 | CAGUAAACUGUUAGAAAA |
| | | G | | C |
| siRNA 4001 | 4001 | UUUUCUAACAGUUUACUG | 10355 | ACAGUAAACUGUUAGAAA |
| | | U | | A |
| siRNA 4002 | 4002 | UUUCUAACAGUUUACUGU | 10356 | AACAGUAAACUGUUAGAA |
| | | U | | A |
| siRNA 4003 | 4003 | UUCUAACAGUUUACUGUU | 10357 | CAACAGUAAACUGUUAGA |
| | | G | | A |
| siRNA 4004 | 4004 | UCUAACAGUUUACUGUUGC | 10358 | GCAACAGUAAACUGUUAG |
| | | | | A |
| siRNA 4005 | 4005 | CUAACAGUUUACUGUUGCU | 10359 | AGCAACAGUAAACUGUUA |
| | | | | G |
| siRNA 4006 | 4006 | UAACAGUUUACUGUUGCU | 10360 | AAGCAACAGUAAACUGUU |
| | | U | | A |
| siRNA 4007 | 4007 | AACAGUUUACUGUUGCUU | 10361 | UAAGCAACAGUAAACUGU |
| | | A | | U |
| siRNA 4008 | 4008 | ACAGUUUACUGUUGCUUA | 10362 | AUAAGCAACAGUAAACUG |
| | | U | | U |
| siRNA 4009 | 4009 | CAGUUUACUGUUGCUUAUC | 10363 | GAUAAGCAACAGUAAACU |
| | | | | G |
| siRNA 4010 | 4010 | AGUUUACUGUUGCUUAUC | 10364 | AGAUAAGCAACAGUAAAC |
| | | U | | U |
| siRNA 4011 | 4011 | GUUUACUGUUGCUUAUCUC | 10365 | GAGAUAAGCAACAGUAAA |
| | | | | C |
| siRNA 4012 | 4012 | UUUACUGUUGCUUAUCUCC | 10366 | GGAGAUAAGCAACAGUAA |
| | | | | A |
| siRNA 4013 | 4013 | UUACUGUUGCUUAUCUCCA | 10367 | UGGAGAUAAGCAACAGUA |
| | | | | A |
| siRNA 4014 | 4014 | UACUGUUGCUUAUCUCCAA | 10368 | UUGGAGAUAAGCAACAGU |
| | | | | A |
| siRNA 4015 | 4015 | ACUGUUGCUUAUCUCCAAG | 10369 | CUUGGAGAUAAGCAACAG |
| | | | | U |
| siRNA 4016 | 4016 | CUGUUGCUUAUCUCCAAGA | 10370 | UCUUGGAGAUAAGCAACA |
| | | | | G |
| siRNA 4017 | 4017 | UGUUGCUUAUCUCCAAGAU | 10371 | AUCUUGGAGAUAAGCAAC |
| | | | | A |
| siRNA 4018 | 4018 | GUUGCUUAUCUCCAAGAUA | 10372 | UAUCUUGGAGAUAAGCAA |
| | | | | C |
| siRNA 4019 | 4019 | UUGCUUAUCUCCAAGAUAU | 10373 | AUAUCUUGGAGAUAAGCA |
| | | | | A |
| siRNA 4020 | 4020 | UGCUUAUCUCCAAGAUAUU | 10374 | AAUAUCUUGGAGAUAAGC |
| | | | | A |
| siRNA 4021 | 4021 | GCUUAUCUCCAAGAUAUUA | 10375 | UAAUAUCUUGGAGAUAAG |
| | | | | C |
| siRNA 4022 | 4022 | CUUAUCUCCAAGAUAUUAU | 10376 | AUAAUAUCUUGGAGAUAA |
| | | | | G |
| siRNA 4023 | 4023 | UUAUCUCCAAGAUAUUAU | 10377 | CAUAAUAUCUUGGAGAUA |
| | | G | | A |
| siRNA 4024 | 4024 | UAUCUCCAAGAUAUUAUG | 10378 | CCAUAAUAUCUUGGAGAU |
| | | G | | A |
| siRNA 4025 | 4025 | AUCUCCAAGAUAUUAUGG | 10379 | UCCAUAAUAUCUUGGAGA |
| | | A | | U |
| siRNA 4026 | 4026 | UCUCCAAGAUAUUAUGGA | 10380 | UUCCAUAAUAUCUUGGAG |
| | | A | | A |
| siRNA 4027 | 4027 | CUCCAAGAUAUUAUGGAA | 10381 | AUUCCAUAAUAUCUUGGA |
| | | U | | G |
| siRNA 4028 | 4028 | UCCAAGAUAUUAUGGAAU | 10382 | AAUUCCAUAAUAUCUUGG |
| | | U | | A |
| siRNA 4029 | 4029 | CCAAGAUAUUAUGGAAUU | 10383 | UAAUUCCAUAAUAUCUUG |
| | | A | | G |
| siRNA 4030 | 4030 | CAAGAUAUUAUGGAAUUA | 10384 | UUAAUUCCAUAAUAUCUU |
| | | A | | G |
| siRNA 4031 | 4031 | AAGAUAUUAUGGAAUUAA | 10385 | CUUAAUUCCAUAAUAUCUU |
| | | G | | |
| siRNA 4032 | 4032 | AGAUAUUAUGGAAUUAAG | 10386 | UCUUAAUUCCAUAAUAUCU |
| | | A | | |
| siRNA 4033 | 4033 | GAUAUUAUGGAAUUAAGA | 10387 | UUCUUAAUUCCAUAAUAUC |
| | | A | | |
| siRNA 4034 | 4034 | AUAUUAUGGAAUUAAGAA | 10388 | AUUCUUAAUUCCAUAAUA |
| | | U | | U |
| siRNA 4035 | 4035 | UAUUAUGGAAUUAAGAAU | 10389 | AAUUCUUAAUUCCAUAAU |
| | | U | | A |
| siRNA 4036 | 4036 | AUUAUGGAAUUAAGAAUU | 10390 | AAAUUCUUAAUUCCAUAA |
| | | U | | U |
| siRNA 4037 | 4037 | UUAUGGAAUUAAGAAUUU | 10391 | AAAAUUCUUAAUUCCAUA |
| | | U | | A |
| siRNA 4038 | 4038 | UAUGGAAUUAAGAAUUUU | 10392 | AAAAAUUCUUAAUUCCAU |
| | | U | | A |
| siRNA 4039 | 4039 | AUGGAAUUAAGAAUUUUU | 10393 | GAAAAAUUCUUAAUUCCA |
| | | C | | U |
| siRNA 4040 | 4040 | UGGAAUUAAGAAUUUUUC | 10394 | GGAAAAAUUCUUAAUUCC |
| | | C | | A |
| siRNA 4041 | 4041 | GGAAUUAAGAAUUUUUCC | 10395 | UGGAAAAAUUCUUAAUUC |
| | | A | | C |
| siRNA 4042 | 4042 | GAAUUAAGAAUUUUUCCA | 10396 | CUGGAAAAAUUCUUAAUU |
| | | G | | C |
| siRNA 4043 | 4043 | AAUUAAGAAUUUUUCCAG | 10397 | UCUGGAAAAAUUCUUAAU |
| | | A | | U |
| siRNA 4044 | 4044 | AUUAAGAAUUUUUCCAGA | 10398 | AUCUGGAAAAAUUCUUAA |
| | | U | | U |
| siRNA 4045 | 4045 | UUAAGAAUUUUUCCAGAU | 10399 | CAUCUGGAAAAAUUCUUA |
| | | G | | A |
| siRNA 4046 | 4046 | UAAGAAUUUUUCCAGAUG | 10400 | UCAUCUGGAAAAAUUCUU |
| | | A | | A |
| siRNA 4047 | 4047 | AAGAAUUUUUCCAGAUGA | 10401 | CUCAUCUGGAAAAAUUCUU |
| | | G | | |
| siRNA 4048 | 4048 | AGAAUUUUUCCAGAUGAG | 10402 | ACUCAUCUGGAAAAAUUCU |
| | | U | | |
| siRNA 4049 | 4049 | GAAUUUUUCCAGAUGAGU | 10403 | CACUCAUCUGGAAAAAUUC |
| | | G | | |
| siRNA 4050 | 4050 | AAUUUUUCCAGAUGAGUG | 10404 | ACACUCAUCUGGAAAAAUU |
| | | U | | |
| siRNA 4051 | 4051 | AUUUUUCCAGAUGAGUGU | 10405 | AACACUCAUCUGGAAAAAU |
| | | U | | |
| siRNA 4052 | 4052 | UUUUUCCAGAUGAGUGUU | 10406 | UAACACUCAUCUGGAAAAA |
| | | A | | |
| siRNA 4053 | 4053 | UUUUCCAGAUGAGUGUUA | 10407 | GUAACACUCAUCUGGAAAA |
| | | C | | |
| siRNA 4054 | 4054 | UUUCCAGAUGAGUGUUAC | 10408 | UGUAACACUCAUCUGGAAA |
| | | A | | |
| siRNA 4055 | 4055 | UUCCAGAUGAGUGUUACA | 10409 | AUGUAACACUCAUCUGGAA |
| | | U | | |
| siRNA 4056 | 4056 | UCCAGAUGAGUGUUACAU | 10410 | UAUGUAACACUCAUCUGGA |
| | | A | | |
| siRNA 4057 | 4057 | CCAGAUGAGUGUUACAUA | 10411 | CUAUGUAACACUCAUCUGG |
| | | G | | |
| siRNA 4058 | 4058 | CAGAUGAGUGUUACAUAG | 10412 | UCUAUGUAACACUCAUCUG |
| | | A | | |
| siRNA 4059 | 4059 | AGAUGAGUGUUACAUAGA | 10413 | AUCUAUGUAACACUCAUCU |
| | | U | | |
| siRNA 4060 | 4060 | GAUGAGUGUUACAUAGAU | 10414 | AAUCUAUGUAACACUCAUC |
| | | U | | |
| siRNA 4061 | 4061 | AUGAGUGUUACAUAGAUU | 10415 | GAAUCUAUGUAACACUCAU |
| | | C | | |
| siRNA 4062 | 4062 | UGAGUGUUACAUAGAUUC | 10416 | AGAAUCUAUGUAACACUCA |
| | | U | | |
| siRNA 4063 | 4063 | GAGUGUUACAUAGAUUCU | 10417 | AAGAAUCUAUGUAACACUC |
| | | U | | |
| siRNA 4064 | 4064 | AGUGUUACAUAGAUUCUU | 10418 | AAAGAAUCUAUGUAACAC |
| | | U | | U |
| siRNA 4065 | 4065 | GUGUUACAUAGAUUCUUU | 10419 | CAAAGAAUCUAUGUAACAC |
| | | G | | |
| siRNA 4066 | 4066 | UGUUACAUAGAUUCUUUG | 10420 | UCAAAGAAUCUAUGUAAC |
| | | A | | A |
| siRNA 4067 | 4067 | GUUACAUAGAUUCUUUGA | 10421 | UUCAAAGAAUCUAUGUAA |
| | | A | | C |
| siRNA 4068 | 4068 | UUACAUAGAUUCUUUGAA | 10422 | AUUCAAAGAAUCUAUGUA |
| | | U | | A |
| siRNA 4069 | 4069 | UACAUAGAUUCUUUGAAU | 10423 | AAUUCAAAGAAUCUAUGU |
| | | U | | A |
| siRNA 4070 | 4070 | ACAUAGAUUCUUUGAAUU | 10424 | AAAUUCAAAGAAUCUAUG |
| | | U | | U |
| siRNA 4071 | 4071 | CAUAGAUUCUUUGAAUUU | 10425 | UAAAUUCAAAGAAUCUAU |
| | | A | | G |
| siRNA 4072 | 4072 | AUAGAUUCUUUGAAUUUA | 10426 | CUAAAUUCAAAGAAUCUA |
| | | G | | U |
| siRNA 4073 | 4073 | UAGAUUCUUUGAAUUUAG | 10427 | ACUAAAUUCAAAGAAUCU |
| | | U | | A |
| siRNA 4074 | 4074 | AGAUUCUUUGAAUUUAGU | 10428 | UACUAAAUUCAAAGAAUC |
| | | A | | U |
| siRNA 4075 | 4075 | GAUUCUUUGAAUUUAGUA | 10429 | AUACUAAAUUCAAAGAAU |
| | | U | | C |
| siRNA 4076 | 4076 | AUUCUUUGAAUUUAGUAU | 10430 | UAUACUAAAUUCAAAGAA |
| | | A | | U |
| siRNA 4077 | 4077 | UUCUUUGAAUUUAGUAUA | 10431 | UUAUACUAAAUUCAAAGA |
| | | A | | A |
| siRNA 4078 | 4078 | UCUUUGAAUUUAGUAUAA | 10432 | UUUAUACUAAAUUCAAAG |
| | | A | | A |
| siRNA 4079 | 4079 | CUUUGAAUUUAGUAUAAA | 10433 | UUUUAUACUAAAUUCAAA |
| | | A | | G |
| siRNA 4080 | 4080 | UUUGAAUUUAGUAUAAAA | 10434 | CUUUUAUACUAAAUUCAA |
| | | G | | A |
| siRNA 4081 | 4081 | UUGAAUUUAGUAUAAAAG | 10435 | ACUUUUAUACUAAAUUCA |
| | | U | | A |
| siRNA 4082 | 4082 | UGAAUUUAGUAUAAAAGU | 10436 | UACUUUUAUACUAAAUUC |
| | | A | | A |
| siRNA 4083 | 4083 | GAAUUUAGUAUAAAAGUA | 10437 | GUACUUUUAUACUAAAUU |
| | | C | | C |
| siRNA 4084 | 4084 | AAUUUAGUAUAAAAGUAC | 10438 | AGUACUUUUAUACUAAAU |
| | | U | | U |
| siRNA 4085 | 4085 | AUUUAGUAUAAAAGUACU | 10439 | CAGUACUUUUAUACUAAA |
| | | G | | U |
| siRNA 4086 | 4086 | UUUAGUAUAAAAGUACUG | 10440 | UCAGUACUUUUAUACUAA |
| | | A | | A |
| siRNA 4087 | 4087 | UUAGUAUAAAAGUACUGA | 10441 | CUCAGUACUUUUAUACUAA |
| | | G | | |
| siRNA 4088 | 4088 | UAGUAUAAAAGUACUGAG | 10442 | UCUCAGUACUUUUAUACUA |
| | | A | | |
| siRNA 4089 | 4089 | AGUAUAAAAGUACUGAGA | 10443 | UUCUCAGUACUUUUAUACU |
| | | A | | |
| siRNA 4090 | 4090 | GUAUAAAAGUACUGAGAA | 10444 | AUUCUCAGUACUUUUAUAC |
| | | U | | |
| siRNA 4091 | 4091 | UAUAAAAGUACUGAGAAU | 10445 | AAUUCUCAGUACUUUUAU |
| | | U | | A |
| siRNA 4092 | 4092 | AUAAAAGUACUGAGAAUU | 10446 | UAAUUCUCAGUACUUUUA |
| | | A | | U |
| siRNA 4093 | 4093 | UAAAAGUACUGAGAAUUA | 10447 | UUAAUUCUCAGUACUUUU |
| | | A | | A |
| siRNA 4094 | 4094 | AAAAGUACUGAGAAUUAA | 10448 | CUUAAUUCUCAGUACUUUU |
| | | G | | |
| siRNA 4095 | 4095 | AAAGUACUGAGAAUUAAG | 10449 | ACUUAAUUCUCAGUACUUU |
| | | U | | |
| siRNA 4096 | 4096 | AAGUACUGAGAAUUAAGU | 10450 | AACUUAAUUCUCAGUACUU |
| | | U | | |
| siRNA 4097 | 4097 | AGUACUGAGAAUUAAGUU | 10451 | AAACUUAAUUCUCAGUACU |
| | | U | | |
| siRNA 4098 | 4098 | GUACUGAGAAUUAAGUUU | 10452 | CAAACUUAAUUCUCAGUAC |
| | | G | | |
| siRNA 4099 | 4099 | UACUGAGAAUUAAGUUUG | 10453 | ACAAACUUAAUUCUCAGUA |
| | | U | | |
| siRNA 4100 | 4100 | ACUGAGAAUUAAGUUUGU | 10454 | UACAAACUUAAUUCUCAGU |
| | | A | | |
| siRNA 4101 | 4101 | CUGAGAAUUAAGUUUGUA | 10455 | GUACAAACUUAAUUCUCAG |
| | | C | | |
| siRNA 4102 | 4102 | UGAGAAUUAAGUUUGUAC | 10456 | AGUACAAACUUAAUUCUCA |
| | | U | | |
| siRNA 4103 | 4103 | GAGAAUUAAGUUUGUACU | 10457 | AAGUACAAACUUAAUUCUC |
| | | U | | |
| siRNA 4104 | 4104 | AGAAUUAAGUUUGUACUU | 10458 | GAAGUACAAACUUAAUUC |
| | | C | | U |
| siRNA 4105 | 4105 | GAAUUAAGUUUGUACUUC | 10459 | GGAAGUACAAACUUAAUU |
| | | C | | C |
| siRNA 4106 | 4106 | AAUUAAGUUUGUACUUCC | 10460 | UGGAAGUACAAACUUAAU |
| | | A | | U |
| siRNA 4107 | 4107 | AUUAAGUUUGUACUUCCA | 10461 | AUGGAAGUACAAACUUAA |
| | | U | | U |
| siRNA 4108 | 4108 | UUAAGUUUGUACUUCCAU | 10462 | UAUGGAAGUACAAACUUA |
| | | A | | A |
| siRNA 4109 | 4109 | UAAGUUUGUACUUCCAUA | 10463 | UUAUGGAAGUACAAACUU |
| | | A | | A |
| siRNA 4110 | 4110 | AAGUUUGUACUUCCAUAA | 10464 | CUUAUGGAAGUACAAACU |
| | | G | | U |
| siRNA 4111 | 4111 | AGUUUGUACUUCCAUAAGC | 10465 | GCUUAUGGAAGUACAAAC |
| | | | | U |
| siRNA 4112 | 4112 | GUUUGUACUUCCAUAAGCU | 10466 | AGCUUAUGGAAGUACAAA |
| | | | | C |
| siRNA 4113 | 4113 | UUUGUACUUCCAUAAGCUU | 10467 | AAGCUUAUGGAAGUACAA |
| | | | | A |
| siRNA 4114 | 4114 | UUGUACUUCCAUAAGCUUG | 10468 | CAAGCUUAUGGAAGUACA |
| | | | | A |
| siRNA 4115 | 4115 | UGUACUUCCAUAAGCUUGG | 10469 | CCAAGCUUAUGGAAGUACA |
| siRNA 4116 | 4116 | GUACUUCCAUAAGCUUGGA | 10470 | UCCAAGCUUAUGGAAGUAC |
| siRNA 4117 | 4117 | UACUUCCAUAAGCUUGGAU | 10471 | AUCCAAGCUUAUGGAAGU |
| | | | | A |
| siRNA 4118 | 4118 | ACUUCCAUAAGCUUGGAUU | 10472 | AAUCCAAGCUUAUGGAAG |
| | | | | U |
| siRNA 4119 | 4119 | CUUCCAUAAGCUUGGAUUU | 10473 | AAAUCCAAGCUUAUGGAA |
| | | | | G |
| siRNA 4120 | 4120 | UUCCAUAAGCUUGGAUUU | 10474 | AAAAUCCAAGCUUAUGGA |
| | | U | | A |
| siRNA 4121 | 4121 | UCCAUAAGCUUGGAUUUU | 10475 | UAAAAUCCAAGCUUAUGG |
| | | A | | A |
| siRNA 4122 | 4122 | CCAUAAGCUUGGAUUUUA | 10476 | UUAAAAUCCAAGCUUAUG |
| | | A | | G |
| siRNA 4123 | 4123 | CAUAAGCUUGGAUUUUAA | 10477 | UUUAAAAUCCAAGCUUAU |
| | | A | | G |
| siRNA 4124 | 4124 | AUAAGCUUGGAUUUUAAA | 10478 | GUUUAAAAUCCAAGCUUA |
| | | C | | U |
| siRNA 4125 | 4125 | UAAGCUUGGAUUUUAAAC | 10479 | UGUUUAAAAUCCAAGCUU |
| | | A | | A |
| siRNA 4126 | 4126 | AAGCUUGGAUUUUAAACA | 10480 | GUGUUUAAAAUCCAAGCU |
| | | C | | U |
| siRNA 4127 | 4127 | AGCUUGGAUUUUAAACAC | 10481 | AGUGUUUAAAAUCCAAGC |
| | | U | | U |
| siRNA 4128 | 4128 | GCUUGGAUUUUAAACACU | 10482 | CAGUGUUUAAAAUCCAAGC |
| | | G | | |
| siRNA 4129 | 4129 | CUUGGAUUUUAAACACUG | 10483 | UCAGUGUUUAAAAUCCAA |
| | | A | | G |
| siRNA 4130 | 4130 | UUGGAUUUUAAACACUGA | 10484 | AUCAGUGUUUAAAAUCCA |
| | | U | | A |
| siRNA 4131 | 4131 | UGGAUUUUAAACACUGAU | 10485 | UAUCAGUGUUUAAAAUCC |
| | | A | | A |
| siRNA 4132 | 4132 | GGAUUUUAAACACUGAUA | 10486 | CUAUCAGUGUUUAAAAUCC |
| | | G | | |
| siRNA 4133 | 4133 | GAUUUUAAACACUGAUAG | 10487 | ACUAUCAGUGUUUAAAAU |
| | | U | | C |
| siRNA 4134 | 4134 | AUUUUAAACACUGAUAGU | 10488 | UACUAUCAGUGUUUAAAA |
| | | A | | U |
| siRNA 4135 | 4135 | UUUUAAACACUGAUAGUA | 10489 | AUACUAUCAGUGUUUAAA |
| | | U | | A |
| siRNA 4136 | 4136 | UUUAAACACUGAUAGUAU | 10490 | GAUACUAUCAGUGUUUAA |
| | | C | | A |
| siRNA 4137 | 4137 | UUAAACACUGAUAGUAUC | 10491 | AGAUACUAUCAGUGUUUA |
| | | U | | A |
| siRNA 4138 | 4138 | UAAACACUGAUAGUAUCUC | 10492 | GAGAUACUAUCAGUGUUU |
| | | | | A |
| siRNA 4139 | 4139 | AAACACUGAUAGUAUCUCA | 10493 | UGAGAUACUAUCAGUGUU |
| | | | | U |
| siRNA 4140 | 4140 | AACACUGAUAGUAUCUCAU | 10494 | AUGAGAUACUAUCAGUGU |
| | | | | U |
| siRNA 4141 | 4141 | ACACUGAUAGUAUCUCAUG | 10495 | CAUGAGAUACUAUCAGUG |
| | | | | U |
| siRNA 4142 | 4142 | CACUGAUAGUAUCUCAUGA | 10496 | UCAUGAGAUACUAUCAGU |
| | | | | G |
| siRNA 4143 | 4143 | ACUGAUAGUAUCUCAUGA | 10497 | CUCAUGAGAUACUAUCAGU |
| | | G | | |
| siRNA 4144 | 4144 | CUGAUAGUAUCUCAUGAG | 10498 | ACUCAUGAGAUACUAUCAG |
| | | U | | |
| siRNA 4145 | 4145 | UGAUAGUAUCUCAUGAGU | 10499 | UACUCAUGAGAUACUAUCA |
| | | A | | |
| siRNA 4146 | 4146 | GAUAGUAUCUCAUGAGUA | 10500 | UUACUCAUGAGAUACUAUC |
| | | A | | |
| siRNA 4147 | 4147 | AUAGUAUCUCAUGAGUAA | 10501 | AUUACUCAUGAGAUACUA |
| | | U | | U |
| siRNA 4148 | 4148 | UAGUAUCUCAUGAGUAAU | 10502 | CAUUACUCAUGAGAUACUA |
| | | G | | |
| siRNA 4149 | 4149 | AGUAUCUCAUGAGUAAUG | 10503 | ACAUUACUCAUGAGAUACU |
| | | U | | |
| siRNA 4150 | 4150 | GUAUCUCAUGAGUAAUGU | 10504 | CACAUUACUCAUGAGAUAC |
| | | G | | |
| siRNA 4151 | 4151 | UAUCUCAUGAGUAAUGUG | 10505 | ACACAUUACUCAUGAGAUA |
| | | U | | |
| siRNA 4152 | 4152 | AUCUCAUGAGUAAUGUGU | 10506 | CACACAUUACUCAUGAGAU |
| | | G | | |
| siRNA 4153 | 4153 | UCUCAUGAGUAAUGUGUG | 10507 | ACACACAUUACUCAUGAGA |
| | | U | | |
| siRNA 4154 | 4154 | CUCAUGAGUAAUGUGUGU | 10508 | AACACACAUUACUCAUGAG |
| | | U | | |
| siRNA 4155 | 4155 | UCAUGAGUAAUGUGUGUU | 10509 | AAACACACAUUACUCAUGA |
| | | U | | |
| siRNA 4156 | 4156 | CAUGAGUAAUGUGUGUUU | 10510 | AAAACACACAUUACUCAUG |
| | | U | | |
| siRNA 4157 | 4157 | AUGAGUAAUGUGUGUUUU | 10511 | CAAAACACACAUUACUCAU |
| | | G | | |
| siRNA 4158 | 4158 | UGAGUAAUGUGUGUUUUG | 10512 | CCAAAACACACAUUACUCA |
| | | G | | |
| siRNA 4159 | 4159 | GAGUAAUGUGUGUUUUGG | 10513 | CCCAAAACACACAUUACUC |
| | | G | | |
| siRNA 4160 | 4160 | AGUAAUGUGUGUUUUGGG | 10514 | UCCCAAAACACACAUUACU |
| | | A | | |
| siRNA 4161 | 4161 | GUAAUGUGUGUUUUGGGA | 10515 | CUCCCAAAACACACAUUAC |
| | | G | | |
| siRNA 4162 | 4162 | UAAUGUGUGUUUUGGGAG | 10516 | UCUCCCAAAACACACAUUA |
| | | A | | |
| siRNA 4163 | 4163 | AAUGUGUGUUUUGGGAGA | 10517 | CUCUCCCAAAACACACAUU |
| | | G | | |
| siRNA 4164 | 4164 | AUGUGUGUUUUGGGAGAG | 10518 | CCUCUCCCAAAACACACAU |
| | | G | | |
| siRNA 4165 | 4165 | UGUGUGUUUUGGGAGAGG | 10519 | CCCUCUCCCAAAACACACA |
| | | G | | |
| siRNA 4166 | 4166 | GUGUGUUUUGGGAGAGGG | 10520 | UCCCUCUCCCAAAACACAC |
| | | A | | |
| siRNA 4167 | 4167 | UGUGUUUUGGGAGAGGGA | 10521 | CUCCCUCUCCCAAAACACA |
| | | G | | |
| siRNA 4168 | 4168 | GUGUUUUGGGAGAGGGAG | 10522 | CCUCCCUCUCCCAAAACAC |
| | | G | | |
| siRNA 4169 | 4169 | UGUUUUGGGAGAGGGAGG | 10523 | CCCUCCCUCUCCCAAAACA |
| | | G | | |
| siRNA 4170 | 4170 | GUUUUGGGAGAGGGAGGG | 10524 | UCCCUCCCUCUCCCAAAAC |
| | | A | | |
| siRNA 4171 | 4171 | UUUUGGGAGAGGGAGGGA | 10525 | AUCCCUCCCUCUCCCAAAA |
| | | U | | |
| siRNA 4172 | 4172 | UUUGGGAGAGGGAGGGAU | 10526 | CAUCCCUCCCUCUCCCAAA |
| | | G | | |
| siRNA 4173 | 4173 | UUGGGAGAGGGAGGGAUG | 10527 | GCAUCCCUCCCUCUCCCAA |
| | | C | | |
| siRNA 4174 | 4174 | UGGGAGAGGGAGGGAUGC | 10528 | AGCAUCCCUCCCUCUCCCA |
| | | U | | |
| siRNA 4175 | 4175 | GGGAGAGGGAGGGAUGCU | 10529 | CAGCAUCCCUCCCUCUCCC |
| | | G | | |
| siRNA 4176 | 4176 | GGAGAGGGAGGGAUGCUG | 10530 | UCAGCAUCCCUCCCUCUCC |
| | | A | | |
| siRNA 4177 | 4177 | GAGAGGGAGGGAUGCUGA | 10531 | AUCAGCAUCCCUCCCUCUC |
| | | U | | |
| siRNA 4178 | 4178 | AGAGGGAGGGAUGCUGAU | 10532 | AAUCAGCAUCCCUCCCUCU |
| | | U | | |
| siRNA 4179 | 4179 | GAGGGAGGGAUGCUGAUU | 10533 | CAAUCAGCAUCCCUCCCUC |
| | | G | | |
| siRNA 4180 | 4180 | AGGGAGGGAUGCUGAUUG | 10534 | UCAAUCAGCAUCCCUCCCU |
| | | A | | |
| siRNA 4181 | 4181 | GGGAGGGAUGCUGAUUGA | 10535 | AUCAAUCAGCAUCCCUCCC |
| | | U | | |
| siRNA 4182 | 4182 | GGAGGGAUGCUGAUUGAU | 10536 | UAUCAAUCAGCAUCCCUCC |
| | | A | | |
| siRNA 4183 | 4183 | GAGGGAUGCUGAUUGAUA | 10537 | AUAUCAAUCAGCAUCCCUC |
| | | U | | |
| siRNA 4184 | 4184 | AGGGAUGCUGAUUGAUAU | 10538 | AAUAUCAAUCAGCAUCCCU |
| | | U | | |
| siRNA 4185 | 4185 | GGGAUGCUGAUUGAUAUU | 10539 | AAAUAUCAAUCAGCAUCCC |
| | | U | | |
| siRNA 4186 | 4186 | GGAUGCUGAUUGAUAUUU | 10540 | GAAAUAUCAAUCAGCAUCC |
| | | C | | |
| siRNA 4187 | 4187 | GAUGCUGAUUGAUAUUUC | 10541 | UGAAAUAUCAAUCAGCAUC |
| | | A | | |
| siRNA 4188 | 4188 | AUGCUGAUUGAUAUUUCA | 10542 | GUGAAAUAUCAAUCAGCA |
| | | C | | U |
| siRNA 4189 | 4189 | UGCUGAUUGAUAUUUCAC | 10543 | UGUGAAAUAUCAAUCAGC |
| | | A | | A |
| siRNA 4190 | 4190 | GCUGAUUGAUAUUUCACA | 10544 | AUGUGAAAUAUCAAUCAG |
| | | U | | C |
| siRNA 4191 | 4191 | CUGAUUGAUAUUUCACAU | 10545 | AAUGUGAAAUAUCAAUCA |
| | | U | | G |
| siRNA 4192 | 4192 | UGAUUGAUAUUUCACAUU | 10546 | CAAUGUGAAAUAUCAAUC |
| | | G | | A |
| siRNA 4193 | 4193 | GAUUGAUAUUUCACAUUG | 10547 | ACAAUGUGAAAUAUCAAU |
| | | U | | C |
| siRNA 4194 | 4194 | AUUGAUAUUUCACAUUGU | 10548 | UACAAUGUGAAAUAUCAA |
| | | A | | U |
| siRNA 4195 | 4195 | UUGAUAUUUCACAUUGUA | 10549 | AUACAAUGUGAAAUAUCA |
| | | U | | A |
| siRNA 4196 | 4196 | UGAUAUUUCACAUUGUAU | 10550 | CAUACAAUGUGAAAUAUC |
| | | G | | A |
| siRNA 4197 | 4197 | GAUAUUUCACAUUGUAUG | 10551 | UCAUACAAUGUGAAAUAU |
| | | A | | C |
| siRNA 4198 | 4198 | AUAUUUCACAUUGUAUGA | 10552 | UUCAUACAAUGUGAAAUA |
| | | A | | U |
| siRNA 4199 | 4199 | UAUUUCACAUUGUAUGAA | 10553 | UUUCAUACAAUGUGAAAU |
| | | A | | A |
| siRNA 4200 | 4200 | AUUUCACAUUGUAUGAAA | 10554 | AUUUCAUACAAUGUGAAA |
| | | U | | U |
| siRNA 4201 | 4201 | UUUCACAUUGUAUGAAAU | 10555 | UAUUUCAUACAAUGUGAA |
| | | A | | A |
| siRNA 4202 | 4202 | UUCACAUUGUAUGAAAUA | 10556 | GUAUUUCAUACAAUGUGA |
| | | C | | A |
| siRNA 4203 | 4203 | UCACAUUGUAUGAAAUACC | 10557 | GGUAUUUCAUACAAUGUG |
| | | | | A |
| siRNA 4204 | 4204 | CACAUUGUAUGAAAUACCA | 10558 | UGGUAUUUCAUACAAUGU |
| | | | | G |
| siRNA 4205 | 4205 | ACAUUGUAUGAAAUACCA | 10559 | AUGGUAUUUCAUACAAUG |
| | | U | | U |
| siRNA 4206 | 4206 | CAUUGUAUGAAAUACCAU | 10560 | CAUGGUAUUUCAUACAAU |
| | | G | | G |
| siRNA 4207 | 4207 | AUUGUAUGAAAUACCAUG | 10561 | ACAUGGUAUUUCAUACAA |
| | | U | | U |
| siRNA 4208 | 4208 | UUGUAUGAAAUACCAUGU | 10562 | AACAUGGUAUUUCAUACA |
| | | U | | A |
| siRNA 4209 | 4209 | UGUAUGAAAUACCAUGUU | 10563 | AAACAUGGUAUUUCAUAC |
| | | U | | A |
| siRNA 4210 | 4210 | GUAUGAAAUACCAUGUUU | 10564 | CAAACAUGGUAUUUCAUAC |
| | | G | | |
| siRNA 4211 | 4211 | UAUGAAAUACCAUGUUUG | 10565 | UCAAACAUGGUAUUUCAU |
| | | A | | A |
| siRNA 4212 | 4212 | AUGAAAUACCAUGUUUGA | 10566 | UUCAAACAUGGUAUUUCA |
| | | A | | U |
| siRNA 4213 | 4213 | UGAAAUACCAUGUUUGAA | 10567 | UUUCAAACAUGGUAUUUC |
| | | A | | A |
| siRNA 4214 | 4214 | GAAAUACCAUGUUUGAAA | 10568 | GUUUCAAACAUGGUAUUU |
| | | C | | C |
| siRNA 4215 | 4215 | AAAUACCAUGUUUGAAAC | 10569 | AGUUUCAAACAUGGUAUU |
| | | U | | U |
| siRNA 4216 | 4216 | AAUACCAUGUUUGAAACUC | 10570 | GAGUUUCAAACAUGGUAU |
| | | | | U |
| siRNA 4217 | 4217 | AUACCAUGUUUGAAACUCA | 10571 | UGAGUUUCAAACAUGGUA |
| | | | | U |
| siRNA 4218 | 4218 | UACCAUGUUUGAAACUCAU | 10572 | AUGAGUUUCAAACAUGGU |
| | | | | A |
| siRNA 4219 | 4219 | ACCAUGUUUGAAACUCAUA | 10573 | UAUGAGUUUCAAACAUGG |
| | | | | U |
| siRNA 4220 | 4220 | CCAUGUUUGAAACUCAUAG | 10574 | CUAUGAGUUUCAAACAUG |
| | | | | G |
| siRNA 4221 | 4221 | CAUGUUUGAAACUCAUAGC | 10575 | GCUAUGAGUUUCAAACAU |
| | | | | G |
| siRNA 4222 | 4222 | AUGUUUGAAACUCAUAGC | 10576 | UGCUAUGAGUUUCAAACA |
| | | A | | U |
| siRNA 4223 | 4223 | UGUUUGAAACUCAUAGCA | 10577 | UUGCUAUGAGUUUCAAAC |
| | | A | | A |
| siRNA 4224 | 4224 | GUUUGAAACUCAUAGCAA | 10578 | AUUGCUAUGAGUUUCAAA |
| | | U | | C |
| siRNA 4225 | 4225 | UUUGAAACUCAUAGCAAU | 10579 | UAUUGCUAUGAGUUUCAA |
| | | A | | A |
| siRNA 4226 | 4226 | UUGAAACUCAUAGCAAUA | 10580 | UUAUUGCUAUGAGUUUCA |
| | | A | | A |
| siRNA 4227 | 4227 | UGAAACUCAUAGCAAUAA | 10581 | AUUAUUGCUAUGAGUUUC |
| | | U | | A |
| siRNA 4228 | 4228 | GAAACUCAUAGCAAUAAU | 10582 | CAUUAUUGCUAUGAGUUU |
| | | G | | C |
| siRNA 4229 | 4229 | AAACUCAUAGCAAUAAUGC | 10583 | GCAUUAUUGCUAUGAGUU |
| | | | | U |
| siRNA 4230 | 4230 | AACUCAUAGCAAUAAUGCU | 10584 | AGCAUUAUUGCUAUGAGU |
| | | | | U |
| siRNA 4231 | 4231 | ACUCAUAGCAAUAAUGCUA | 10585 | UAGCAUUAUUGCUAUGAG |
| | | | | U |
| siRNA 4232 | 4232 | CUCAUAGCAAUAAUGCUAU | 10586 | AUAGCAUUAUUGCUAUGA |
| | | | | G |
| siRNA 4233 | 4233 | UCAUAGCAAUAAUGCUAU | 10587 | CAUAGCAUUAUUGCUAUG |
| | | G | | A |
| siRNA 4234 | 4234 | CAUAGCAAUAAUGCUAUGC | 10588 | GCAUAGCAUUAUUGCUAU |
| | | | | G |
| siRNA 4235 | 4235 | AUAGCAAUAAUGCUAUGC | 10589 | AGCAUAGCAUUAUUGCUA |
| | | U | | U |
| siRNA 4236 | 4236 | UAGCAAUAAUGCUAUGCU | 10590 | CAGCAUAGCAUUAUUGCUA |
| | | G | | |
| siRNA 4237 | 4237 | AGCAAUAAUGCUAUGCUG | 10591 | ACAGCAUAGCAUUAUUGCU |
| | | U | | |
| siRNA 4238 | 4238 | GCAAUAAUGCUAUGCUGU | 10592 | AACAGCAUAGCAUUAUUGC |
| | | U | | |
| siRNA 4239 | 4239 | CAAUAAUGCUAUGCUGUU | 10593 | CAACAGCAUAGCAUUAUUG |
| | | G | | |
| siRNA 4240 | 4240 | AAUAAUGCUAUGCUGUUG | 10594 | ACAACAGCAUAGCAUUAUU |
| | | U | | |
| siRNA 4241 | 4241 | AUAAUGCUAUGCUGUUGU | 10595 | CACAACAGCAUAGCAUUAU |
| | | G | | |
| siRNA 4242 | 4242 | UAAUGCUAUGCUGUUGUG | 10596 | UCACAACAGCAUAGCAUUA |
| | | A | | |
| siRNA 4243 | 4243 | AAUGCUAUGCUGUUGUGA | 10597 | AUCACAACAGCAUAGCAUU |
| | | U | | |
| siRNA 4244 | 4244 | AUGCUAUGCUGUUGUGAU | 10598 | GAUCACAACAGCAUAGCAU |
| | | C | | |
| siRNA 4245 | 4245 | UGCUAUGCUGUUGUGAUCC | 10599 | GGAUCACAACAGCAUAGCA |
| siRNA 4246 | 4246 | GCUAUGCUGUUGUGAUCCC | 10600 | GGGAUCACAACAGCAUAGC |
| siRNA 4247 | 4247 | CUAUGCUGUUGUGAUCCCU | 10601 | AGGGAUCACAACAGCAUAG |
| siRNA 4248 | 4248 | UAUGCUGUUGUGAUCCCUC | 10602 | GAGGGAUCACAACAGCAUA |
| siRNA 4249 | 4249 | AUGCUGUUGUGAUCCCUCU | 10603 | AGAGGGAUCACAACAGCAU |
| siRNA 4250 | 4250 | UGCUGUUGUGAUCCCUCUC | 10604 | GAGAGGGAUCACAACAGCA |
| siRNA 4251 | 4251 | GCUGUUGUGAUCCCUCUCA | 10605 | UGAGAGGGAUCACAACAGC |
| siRNA 4252 | 4252 | CUGUUGUGAUCCCUCUCAA | 10606 | UUGAGAGGGAUCACAACA |
| | | | | G |
| siRNA 4253 | 4253 | UGUUGUGAUCCCUCUCAAG | 10607 | CUUGAGAGGGAUCACAACA |
| siRNA 4254 | 4254 | GUUGUGAUCCCUCUCAAGU | 10608 | ACUUGAGAGGGAUCACAAC |
| siRNA 4255 | 4255 | UUGUGAUCCCUCUCAAGUU | 10609 | AACUUGAGAGGGAUCACA |
| | | | | A |
| siRNA 4256 | 4256 | UGUGAUCCCUCUCAAGUUC | 10610 | GAACUUGAGAGGGAUCAC |
| | | | | A |
| siRNA 4257 | 4257 | GUGAUCCCUCUCAAGUUCU | 10611 | AGAACUUGAGAGGGAUCA |
| | | | | C |
| siRNA 4258 | 4258 | UGAUCCCUCUCAAGUUCUG | 10612 | CAGAACUUGAGAGGGAUC |
| | | | | A |
| siRNA 4259 | 4259 | GAUCCCUCUCAAGUUCUGC | 10613 | GCAGAACUUGAGAGGGAU |
| | | | | C |
| siRNA 4260 | 4260 | AUCCCUCUCAAGUUCUGCA | 10614 | UGCAGAACUUGAGAGGGA |
| | | | | U |
| siRNA 4261 | 4261 | UCCCUCUCAAGUUCUGCAU | 10615 | AUGCAGAACUUGAGAGGG |
| | | | | A |
| siRNA 4262 | 4262 | CCCUCUCAAGUUCUGCAUU | 10616 | AAUGCAGAACUUGAGAGG |
| | | | | G |
| siRNA 4263 | 4263 | CCUCUCAAGUUCUGCAUUU | 10617 | AAAUGCAGAACUUGAGAG |
| | | | | G |
| siRNA 4264 | 4264 | CUCUCAAGUUCUGCAUUUA | 10618 | UAAAUGCAGAACUUGAGA |
| | | | | G |
| siRNA 4265 | 4265 | UCUCAAGUUCUGCAUUUAA | 10619 | UUAAAUGCAGAACUUGAG |
| | | | | A |
| siRNA 4266 | 4266 | CUCAAGUUCUGCAUUUAAA | 10620 | UUUAAAUGCAGAACUUGA |
| | | | | G |
| siRNA 4267 | 4267 | UCAAGUUCUGCAUUUAAA | 10621 | UUUUAAAUGCAGAACUUG |
| | | A | | A |
| siRNA 4268 | 4268 | CAAGUUCUGCAUUUAAAA | 10622 | AUUUUAAAUGCAGAACUU |
| | | U | | G |
| siRNA 4269 | 4269 | AAGUUCUGCAUUUAAAAU | 10623 | UAUUUUAAAUGCAGAACU |
| | | A | | U |
| siRNA 4270 | 4270 | AGUUCUGCAUUUAAAAUA | 10624 | AUAUUUUAAAUGCAGAAC |
| | | U | | U |
| siRNA 4271 | 4271 | GUUCUGCAUUUAAAAUAU | 10625 | UAUAUUUUAAAUGCAGAA |
| | | A | | C |
| siRNA 4272 | 4272 | UUCUGCAUUUAAAAUAUA | 10626 | AUAUAUUUUAAAUGCAGA |
| | | U | | A |
| siRNA 4273 | 4273 | UCUGCAUUUAAAAUAUAU | 10627 | AAUAUAUUUUAAAUGCAG |
| | | U | | A |
| siRNA 4274 | 4274 | CUGCAUUUAAAAUAUAUU | 10628 | AAAUAUAUUUUAAAUGCA |
| | | U | | G |
| siRNA 4275 | 4275 | UGCAUUUAAAAUAUAUUU | 10629 | AAAAUAUAUUUUAAAUGC |
| | | U | | A |
| siRNA 4276 | 4276 | GCAUUUAAAAUAUAUUUU | 10630 | AAAAAUAUAUUUUAAAUG |
| | | U | | C |
| siRNA 4277 | 4277 | CAUUUAAAAUAUAUUUUU | 10631 | AAAAAAUAUAUUUUAAAU |
| | | U | | G |
| siRNA 4278 | 4278 | AUUUAAAAUAUAUUUUUU | 10632 | GAAAAAAUAUAUUUUAAA |
| | | C | | U |
| siRNA 4279 | 4279 | UUUAAAAUAUAUUUUUUC | 10633 | AGAAAAAAUAUAUUUUAA |
| | | U | | A |
| siRNA 4280 | 4280 | UUAAAAUAUAUUUUUUCU | 10634 | AAGAAAAAAUAUAUUUUA |
| | | U | | A |
| siRNA 4281 | 4281 | UAAAAUAUAUUUUUUCUU | 10635 | AAAGAAAAAAUAUAUUUU |
| | | U | | A |
| siRNA 4282 | 4282 | AAAAUAUAUUUUUUCUUU | 10636 | UAAAGAAAAAAUAUAUUU |
| | | A | | U |
| siRNA 4283 | 4283 | AAAUAUAUUUUUUCUUUA | 10637 | AUAAAGAAAAAAUAUAUU |
| | | U | | U |
| siRNA 4284 | 4284 | AAUAUAUUUUUUCUUUAU | 10638 | UAUAAAGAAAAAAUAUAU |
| | | A | | U |
| siRNA 4285 | 4285 | AUAUAUUUUUUCUUUAUA | 10639 | CUAUAAAGAAAAAAUAUA |
| | | G | | U |
| siRNA 4286 | 4286 | UAUAUUUUUUCUUUAUAG | 10640 | CCUAUAAAGAAAAAAUAU |
| | | G | | A |
| siRNA 4287 | 4287 | AUAUUUUUUCUUUAUAGG | 10641 | UCCUAUAAAGAAAAAAUA |
| | | A | | U |
| siRNA 4288 | 4288 | UAUUUUUUCUUUAUAGGA | 10642 | UUCCUAUAAAGAAAAAAU |
| | | A | | A |
| siRNA 4289 | 4289 | AUUUUUUCUUUAUAGGAA | 10643 | AUUCCUAUAAAGAAAAAA |
| | | U | | U |
| siRNA 4290 | 4290 | UUUUUUCUUUAUAGGAAU | 10644 | AAUUCCUAUAAAGAAAAA |
| | | U | | A |
| siRNA 4291 | 4291 | UUUUUCUUUAUAGGAAUU | 10645 | CAAUUCCUAUAAAGAAAA |
| | | G | | A |
| siRNA 4292 | 4292 | UUUUCUUUAUAGGAAUUG | 10646 | UCAAUUCCUAUAAAGAAA |
| | | A | | A |
| siRNA 4293 | 4293 | UUUCUUUAUAGGAAUUGA | 10647 | AUCAAUUCCUAUAAAGAA |
| | | U | | A |
| siRNA 4294 | 4294 | UUCUUUAUAGGAAUUGAU | 10648 | CAUCAAUUCCUAUAAAGAA |
| | | G | | |
| siRNA 4295 | 4295 | UCUUUAUAGGAAUUGAUG | 10649 | ACAUCAAUUCCUAUAAAGA |
| | | U | | |
| siRNA 4296 | 4296 | CUUUAUAGGAAUUGAUGU | 10650 | UACAUCAAUUCCUAUAAAG |
| | | A | | |
| siRNA 4297 | 4297 | UUUAUAGGAAUUGAUGUA | 10651 | AUACAUCAAUUCCUAUAAA |
| | | U | | |
| siRNA 4298 | 4298 | UUAUAGGAAUUGAUGUAU | 10652 | UAUACAUCAAUUCCUAUAA |
| | | A | | |
| siRNA 4299 | 4299 | UAUAGGAAUUGAUGUAUA | 10653 | GUAUACAUCAAUUCCUAUA |
| | | C | | |
| siRNA 4300 | 4300 | AUAGGAAUUGAUGUAUAC | 10654 | GGUAUACAUCAAUUCCUAU |
| | | C | | |
| siRNA 4301 | 4301 | UAGGAAUUGAUGUAUACC | 10655 | UGGUAUACAUCAAUUCCUA |
| | | A | | |
| siRNA 4302 | 4302 | AGGAAUUGAUGUAUACCA | 10656 | AUGGUAUACAUCAAUUCCU |
| | | U | | |
| siRNA 4303 | 4303 | GGAAUUGAUGUAUACCAU | 10657 | CAUGGUAUACAUCAAUUCC |
| | | G | | |
| siRNA 4304 | 4304 | GAAUUGAUGUAUACCAUG | 10658 | UCAUGGUAUACAUCAAUUC |
| | | A | | |
| siRNA 4305 | 4305 | AAUUGAUGUAUACCAUGA | 10659 | UUCAUGGUAUACAUCAAU |
| | | A | | U |
| siRNA 4306 | 4306 | AUUGAUGUAUACCAUGAA | 10660 | CUUCAUGGUAUACAUCAAU |
| | | G | | |
| siRNA 4307 | 4307 | UUGAUGUAUACCAUGAAG | 10661 | ACUUCAUGGUAUACAUCAA |
| | | U | | |
| siRNA 4308 | 4308 | UGAUGUAUACCAUGAAGU | 10662 | GACUUCAUGGUAUACAUCA |
| | | C | | |
| siRNA 4309 | 4309 | GAUGUAUACCAUGAAGUC | 10663 | UGACUUCAUGGUAUACAUC |
| | | A | | |
| siRNA 4310 | 4310 | AUGUAUACCAUGAAGUCA | 10664 | AUGACUUCAUGGUAUACA |
| | | U | | U |
| siRNA 4311 | 4311 | UGUAUACCAUGAAGUCAU | 10665 | AAUGACUUCAUGGUAUAC |
| | | U | | A |
| siRNA 4312 | 4312 | GUAUACCAUGAAGUCAUU | 10666 | CAAUGACUUCAUGGUAUAC |
| | | G | | |
| siRNA 4313 | 4313 | UAUACCAUGAAGUCAUUG | 10667 | ACAAUGACUUCAUGGUAU |
| | | U | | A |
| siRNA 4314 | 4314 | AUACCAUGAAGUCAUUGUC | 10668 | GACAAUGACUUCAUGGUA |
| | | | | U |
| siRNA 4315 | 4315 | UACCAUGAAGUCAUUGUCA | 10669 | UGACAAUGACUUCAUGGU |
| | | | | A |
| siRNA 4316 | 4316 | ACCAUGAAGUCAUUGUCAG | 10670 | CUGACAAUGACUUCAUGGU |
| siRNA 4317 | 4317 | CCAUGAAGUCAUUGUCAGU | 10671 | ACUGACAAUGACUUCAUGG |
| siRNA 4318 | 4318 | CAUGAAGUCAUUGUCAGU | 10672 | AACUGACAAUGACUUCAUG |
| | | U | | |
| siRNA 4319 | 4319 | AUGAAGUCAUUGUCAGUU | 10673 | CAACUGACAAUGACUUCAU |
| | | G | | |
| siRNA 4320 | 4320 | UGAAGUCAUUGUCAGUUG | 10674 | ACAACUGACAAUGACUUCA |
| | | U | | |
| siRNA 4321 | 4321 | GAAGUCAUUGUCAGUUGU | 10675 | UACAACUGACAAUGACUUC |
| | | A | | |
| siRNA 4322 | 4322 | AAGUCAUUGUCAGUUGUA | 10676 | CUACAACUGACAAUGACUU |
| | | G | | |
| siRNA 4323 | 4323 | AGUCAUUGUCAGUUGUAG | 10677 | ACUACAACUGACAAUGACU |
| | | U | | |
| siRNA 4324 | 4324 | GUCAUUGUCAGUUGUAGU | 10678 | UACUACAACUGACAAUGAC |
| | | A | | |
| siRNA 4325 | 4325 | UCAUUGUCAGUUGUAGUA | 10679 | CUACUACAACUGACAAUGA |
| | | G | | |
| siRNA 4326 | 4326 | CAUUGUCAGUUGUAGUAG | 10680 | GCUACUACAACUGACAAUG |
| | | C | | |
| siRNA 4327 | 4327 | AUUGUCAGUUGUAGUAGC | 10681 | AGCUACUACAACUGACAAU |
| | | U | | |
| siRNA 4328 | 4328 | UUGUCAGUUGUAGUAGCU | 10682 | GAGCUACUACAACUGACAA |
| | | C | | |
| siRNA 4329 | 4329 | UGUCAGUUGUAGUAGCUC | 10683 | AGAGCUACUACAACUGACA |
| | | U | | |
| siRNA 4330 | 4330 | GUCAGUUGUAGUAGCUCU | 10684 | CAGAGCUACUACAACUGAC |
| | | G | | |
| siRNA 4331 | 4331 | UCAGUUGUAGUAGCUCUG | 10685 | UCAGAGCUACUACAACUGA |
| | | A | | |
| siRNA 4332 | 4332 | CAGUUGUAGUAGCUCUGA | 10686 | AUCAGAGCUACUACAACUG |
| | | U | | |
| siRNA 4333 | 4333 | AGUUGUAGUAGCUCUGAU | 10687 | CAUCAGAGCUACUACAACU |
| | | G | | |
| siRNA 4334 | 4334 | GUUGUAGUAGCUCUGAUG | 10688 | ACAUCAGAGCUACUACAAC |
| | | U | | |
| siRNA 4335 | 4335 | UUGUAGUAGCUCUGAUGU | 10689 | AACAUCAGAGCUACUACAA |
| | | U | | |
| siRNA 4336 | 4336 | UGUAGUAGCUCUGAUGUU | 10690 | CAACAUCAGAGCUACUACA |
| | | G | | |
| siRNA 4337 | 4337 | GUAGUAGCUCUGAUGUUG | 10691 | UCAACAUCAGAGCUACUAC |
| | | A | | |
| siRNA 4338 | 4338 | UAGUAGCUCUGAUGUUGA | 10692 | UUCAACAUCAGAGCUACUA |
| | | A | | |
| siRNA 4339 | 4339 | AGUAGCUCUGAUGUUGAA | 10693 | AUUCAACAUCAGAGCUACU |
| | | U | | |
| siRNA 4340 | 4340 | GUAGCUCUGAUGUUGAAU | 10694 | CAUUCAACAUCAGAGCUAC |
| | | G | | |
| siRNA 4341 | 4341 | UAGCUCUGAUGUUGAAUG | 10695 | UCAUUCAACAUCAGAGCUA |
| | | A | | |
| siRNA 4342 | 4342 | AGCUCUGAUGUUGAAUGA | 10696 | CUCAUUCAACAUCAGAGCU |
| | | G | | |
| siRNA 4343 | 4343 | GCUCUGAUGUUGAAUGAG | 10697 | UCUCAUUCAACAUCAGAGC |
| | | A | | |
| siRNA 4344 | 4344 | CUCUGAUGUUGAAUGAGA | 10698 | AUCUCAUUCAACAUCAGAG |
| | | U | | |
| siRNA 4345 | 4345 | UCUGAUGUUGAAUGAGAU | 10699 | UAUCUCAUUCAACAUCAGA |
| | | A | | |
| siRNA 4346 | 4346 | CUGAUGUUGAAUGAGAUA | 10700 | AUAUCUCAUUCAACAUCAG |
| | | U | | |
| siRNA 4347 | 4347 | UGAUGUUGAAUGAGAUAU | 10701 | GAUAUCUCAUUCAACAUCA |
| | | C | | |
| siRNA 4348 | 4348 | GAUGUUGAAUGAGAUAUC | 10702 | UGAUAUCUCAUUCAACAUC |
| | | A | | |
| siRNA 4349 | 4349 | AUGUUGAAUGAGAUAUCA | 10703 | AUGAUAUCUCAUUCAACAU |
| | | U | | |
| siRNA 4350 | 4350 | UGUUGAAUGAGAUAUCAU | 10704 | CAUGAUAUCUCAUUCAACA |
| | | G | | |
| siRNA 4351 | 4351 | GUUGAAUGAGAUAUCAUG | 10705 | ACAUGAUAUCUCAUUCAAC |
| | | U | | |
| siRNA 4352 | 4352 | UUGAAUGAGAUAUCAUGU | 10706 | AACAUGAUAUCUCAUUCAA |
| | | U | | |
| siRNA 4353 | 4353 | UGAAUGAGAUAUCAUGUU | 10707 | AAACAUGAUAUCUCAUUCA |
| | | U | | |
| siRNA 4354 | 4354 | GAAUGAGAUAUCAUGUUU | 10708 | AAAACAUGAUAUCUCAUUC |
| | | U | | |
| siRNA 4355 | 4355 | AAUGAGAUAUCAUGUUUU | 10709 | UAAAACAUGAUAUCUCAU |
| | | A | | U |
| siRNA 4356 | 4356 | AUGAGAUAUCAUGUUUUA | 10710 | CUAAAACAUGAUAUCUCAU |
| | | G | | |
| siRNA 4357 | 4357 | UGAGAUAUCAUGUUUUAG | 10711 | GCUAAAACAUGAUAUCUCA |
| | | C | | |
| siRNA 4358 | 4358 | GAGAUAUCAUGUUUUAGC | 10712 | UGCUAAAACAUGAUAUCUC |
| | | A | | |
| siRNA 4359 | 4359 | AGAUAUCAUGUUUUAGCA | 10713 | AUGCUAAAACAUGAUAUC |
| | | U | | U |
| siRNA 4360 | 4360 | GAUAUCAUGUUUUAGCAU | 10714 | AAUGCUAAAACAUGAUAU |
| | | U | | C |
| siRNA 4361 | 4361 | AUAUCAUGUUUUAGCAUU | 10715 | GAAUGCUAAAACAUGAUA |
| | | C | | U |
| siRNA 4362 | 4362 | UAUCAUGUUUUAGCAUUCC | 10716 | GGAAUGCUAAAACAUGAU |
| | | | | A |
| siRNA 4363 | 4363 | AUCAUGUUUUAGCAUUCCA | 10717 | UGGAAUGCUAAAACAUGA |
| | | | | U |
| siRNA 4364 | 4364 | UCAUGUUUUAGCAUUCCAU | 10718 | AUGGAAUGCUAAAACAUG |
| | | | | A |
| siRNA 4365 | 4365 | CAUGUUUUAGCAUUCCAUU | 10719 | AAUGGAAUGCUAAAACAU |
| | | | | G |
| siRNA 4366 | 4366 | AUGUUUUAGCAUUCCAUU | 10720 | AAAUGGAAUGCUAAAACA |
| | | U | | U |
| siRNA 4367 | 4367 | UGUUUUAGCAUUCCAUUU | 10721 | AAAAUGGAAUGCUAAAAC |
| | | U | | A |
| siRNA 4368 | 4368 | GUUUUAGCAUUCCAUUUU | 10722 | UAAAAUGGAAUGCUAAAA |
| | | A | | C |
| siRNA 4369 | 4369 | UUUUAGCAUUCCAUUUUAC | 10723 | GUAAAAUGGAAUGCUAAA |
| | | | | A |
| siRNA 4370 | 4370 | UUUAGCAUUCCAUUUUACU | 10724 | AGUAAAAUGGAAUGCUAA |
| | | | | A |
| siRNA 4371 | 4371 | UUAGCAUUCCAUUUUACUG | 10725 | CAGUAAAAUGGAAUGCUA |
| | | | | A |
| siRNA 4372 | 4372 | UAGCAUUCCAUUUUACUGA | 10726 | UCAGUAAAAUGGAAUGCU |
| | | | | A |
| siRNA 4373 | 4373 | AGCAUUCCAUUUUACUGAC | 10727 | GUCAGUAAAAUGGAAUGC |
| | | | | U |
| siRNA 4374 | 4374 | GCAUUCCAUUUUACUGACU | 10728 | AGUCAGUAAAAUGGAAUG |
| | | | | C |
| siRNA 4375 | 4375 | CAUUCCAUUUUACUGACUA | 10729 | UAGUCAGUAAAAUGGAAU |
| | | | | G |
| siRNA 4376 | 4376 | AUUCCAUUUUACUGACUAG | 10730 | CUAGUCAGUAAAAUGGAA |
| | | | | U |
| siRNA 4377 | 4377 | UUCCAUUUUACUGACUAGG | 10731 | CCUAGUCAGUAAAAUGGA |
| | | | | A |
| siRNA 4378 | 4378 | UCCAUUUUACUGACUAGGG | 10732 | CCCUAGUCAGUAAAAUGGA |
| siRNA 4379 | 4379 | CCAUUUUACUGACUAGGGU | 10733 | ACCCUAGUCAGUAAAAUGG |
| siRNA 4380 | 4380 | CAUUUUACUGACUAGGGU | 10734 | UACCCUAGUCAGUAAAAUG |
| | | A | | |
| siRNA 4381 | 4381 | AUUUUACUGACUAGGGUA | 10735 | CUACCCUAGUCAGUAAAAU |
| | | G | | |
| siRNA 4382 | 4382 | UUUUACUGACUAGGGUAG | 10736 | UCUACCCUAGUCAGUAAAA |
| | | A | | |
| siRNA 4383 | 4383 | UUUACUGACUAGGGUAGA | 10737 | UUCUACCCUAGUCAGUAAA |
| | | A | | |
| siRNA 4384 | 4384 | UUACUGACUAGGGUAGAA | 10738 | CUUCUACCCUAGUCAGUAA |
| | | G | | |
| siRNA 4385 | 4385 | UACUGACUAGGGUAGAAG | 10739 | UCUUCUACCCUAGUCAGUA |
| | | A | | |
| siRNA 4386 | 4386 | ACUGACUAGGGUAGAAGA | 10740 | UUCUUCUACCCUAGUCAGU |
| | | A | | |
| siRNA 4387 | 4387 | CUGACUAGGGUAGAAGAA | 10741 | GUUCUUCUACCCUAGUCAG |
| | | C | | |
| siRNA 4388 | 4388 | UGACUAGGGUAGAAGAAC | 10742 | UGUUCUUCUACCCUAGUCA |
| | | A | | |
| siRNA 4389 | 4389 | GACUAGGGUAGAAGAACA | 10743 | GUGUUCUUCUACCCUAGUC |
| | | C | | |
| siRNA 4390 | 4390 | ACUAGGGUAGAAGAACAC | 10744 | AGUGUUCUUCUACCCUAGU |
| | | U | | |
| siRNA 4391 | 4391 | CUAGGGUAGAAGAACACU | 10745 | AAGUGUUCUUCUACCCUAG |
| | | U | | |
| siRNA 4392 | 4392 | UAGGGUAGAAGAACACUU | 10746 | AAAGUGUUCUUCUACCCUA |
| | | U | | |
| siRNA 4393 | 4393 | AGGGUAGAAGAACACUUU | 10747 | AAAAGUGUUCUUCUACCCU |
| | | U | | |
| siRNA 4394 | 4394 | GGGUAGAAGAACACUUUU | 10748 | GAAAAGUGUUCUUCUACCC |
| | | C | | |
| siRNA 4395 | 4395 | GGUAGAAGAACACUUUUC | 10749 | AGAAAAGUGUUCUUCUACC |
| | | U | | |
| siRNA 4396 | 4396 | GUAGAAGAACACUUUUCU | 10750 | AAGAAAAGUGUUCUUCUA |
| | | U | | C |
| siRNA 4397 | 4397 | UAGAAGAACACUUUUCUU | 10751 | CAAGAAAAGUGUUCUUCU |
| | | G | | A |
| siRNA 4398 | 4398 | AGAAGAACACUUUUCUUG | 10752 | CCAAGAAAAGUGUUCUUCU |
| | | G | | |
| siRNA 4399 | 4399 | GAAGAACACUUUUCUUGGC | 10753 | GCCAAGAAAAGUGUUCUUC |
| siRNA 4400 | 4400 | AAGAACACUUUUCUUGGCU | 10754 | AGCCAAGAAAAGUGUUCU |
| | | | | U |
| siRNA 4401 | 4401 | AGAACACUUUUCUUGGCUA | 10755 | UAGCCAAGAAAAGUGUUC |
| | | | | U |
| siRNA 4402 | 4402 | GAACACUUUUCUUGGCUAC | 10756 | GUAGCCAAGAAAAGUGUU |
| | | | | C |
| siRNA 4403 | 4403 | AACACUUUUCUUGGCUACA | 10757 | UGUAGCCAAGAAAAGUGU |
| | | | | U |
| siRNA 4404 | 4404 | ACACUUUUCUUGGCUACAU | 10758 | AUGUAGCCAAGAAAAGUG |
| | | | | U |
| siRNA 4405 | 4405 | CACUUUUCUUGGCUACAUU | 10759 | AAUGUAGCCAAGAAAAGU |
| | | | | G |
| siRNA 4406 | 4406 | ACUUUUCUUGGCUACAUUU | 10760 | AAAUGUAGCCAAGAAAAG |
| | | | | U |
| siRNA 4407 | 4407 | CUUUUCUUGGCUACAUUUG | 10761 | CAAAUGUAGCCAAGAAAA |
| | | | | G |
| siRNA 4408 | 4408 | UUUUCUUGGCUACAUUUG | 10762 | CCAAAUGUAGCCAAGAAAA |
| | | G | | |
| siRNA 4409 | 4409 | UUUCUUGGCUACAUUUGG | 10763 | UCCAAAUGUAGCCAAGAAA |
| | | A | | |
| siRNA 4410 | 4410 | UUCUUGGCUACAUUUGGA | 10764 | CUCCAAAUGUAGCCAAGAA |
| | | G | | |
| siRNA 4411 | 4411 | UCUUGGCUACAUUUGGAG | 10765 | CCUCCAAAUGUAGCCAAGA |
| | | G | | |
| siRNA 4412 | 4412 | CUUGGCUACAUUUGGAGG | 10766 | UCCUCCAAAUGUAGCCAAG |
| | | A | | |
| siRNA 4413 | 4413 | UUGGCUACAUUUGGAGGA | 10767 | AUCCUCCAAAUGUAGCCAA |
| | | U | | |
| siRNA 4414 | 4414 | UGGCUACAUUUGGAGGAU | 10768 | UAUCCUCCAAAUGUAGCCA |
| | | A | | |
| siRNA 4415 | 4415 | GGCUACAUUUGGAGGAUA | 10769 | GUAUCCUCCAAAUGUAGCC |
| | | C | | |
| siRNA 4416 | 4416 | GCUACAUUUGGAGGAUACC | 10770 | GGUAUCCUCCAAAUGUAGC |
| siRNA 4417 | 4417 | CUACAUUUGGAGGAUACCC | 10771 | GGGUAUCCUCCAAAUGUAG |
| siRNA 4418 | 4418 | UACAUUUGGAGGAUACCCA | 10772 | UGGGUAUCCUCCAAAUGUA |
| siRNA 4419 | 4419 | ACAUUUGGAGGAUACCCAG | 10773 | CUGGGUAUCCUCCAAAUGU |
| siRNA 4420 | 4420 | CAUUUGGAGGAUACCCAGG | 10774 | CCUGGGUAUCCUCCAAAUG |
| siRNA 4421 | 4421 | AUUUGGAGGAUACCCAGG | 10775 | CCCUGGGUAUCCUCCAAAU |
| | | G | | |
| siRNA 4422 | 4422 | UUUGGAGGAUACCCAGGG | 10776 | UCCCUGGGUAUCCUCCAAA |
| | | A | | |
| siRNA 4423 | 4423 | UUGGAGGAUACCCAGGGA | 10777 | CUCCCUGGGUAUCCUCCAA |
| | | G | | |
| siRNA 4424 | 4424 | UGGAGGAUACCCAGGGAG | 10778 | ACUCCCUGGGUAUCCUCCA |
| | | U | | |
| siRNA 4425 | 4425 | GGAGGAUACCCAGGGAGUC | 10779 | GACUCCCUGGGUAUCCUCC |
| siRNA 4426 | 4426 | GAGGAUACCCAGGGAGUCU | 10780 | AGACUCCCUGGGUAUCCUC |
| siRNA 4427 | 4427 | AGGAUACCCAGGGAGUCUU | 10781 | AAGACUCCCUGGGUAUCCU |
| siRNA 4428 | 4428 | GGAUACCCAGGGAGUCUUG | 10782 | CAAGACUCCCUGGGUAUCC |
| siRNA 4429 | 4429 | GAUACCCAGGGAGUCUUGG | 10783 | CCAAGACUCCCUGGGUAUC |
| siRNA 4430 | 4430 | AUACCCAGGGAGUCUUGGG | 10784 | CCCAAGACUCCCUGGGUAU |
| siRNA 4431 | 4431 | UACCCAGGGAGUCUUGGGU | 10785 | ACCCAAGACUCCCUGGGUA |
| siRNA 4432 | 4432 | ACCCAGGGAGUCUUGGGUG | 10786 | CACCCAAGACUCCCUGGGU |
| siRNA 4433 | 4433 | CCCAGGGAGUCUUGGGUGU | 10787 | ACACCCAAGACUCCCUGGG |
| siRNA 4434 | 4434 | CCAGGGAGUCUUGGGUGU | 10788 | AACACCCAAGACUCCCUGG |
| | | U | | |
| siRNA 4435 | 4435 | CAGGGAGUCUUGGGUGUU | 10789 | GAACACCCAAGACUCCCUG |
| | | C | | |
| siRNA 4436 | 4436 | AGGGAGUCUUGGGUGUUC | 10790 | GGAACACCCAAGACUCCCU |
| | | C | | |
| siRNA 4437 | 4437 | GGGAGUCUUGGGUGUUCC | 10791 | AGGAACACCCAAGACUCCC |
| | | U | | |
| siRNA 4438 | 4438 | GGAGUCUUGGGUGUUCCU | 10792 | AAGGAACACCCAAGACUCC |
| | | U | | |
| siRNA 4439 | 4439 | GAGUCUUGGGUGUUCCUU | 10793 | UAAGGAACACCCAAGACUC |
| | | A | | |
| siRNA 4440 | 4440 | AGUCUUGGGUGUUCCUUA | 10794 | AUAAGGAACACCCAAGACU |
| | | U | | |
| siRNA 4441 | 4441 | GUCUUGGGUGUUCCUUAUC | 10795 | GAUAAGGAACACCCAAGAC |
| siRNA 4442 | 4442 | UCUUGGGUGUUCCUUAUCU | 10796 | AGAUAAGGAACACCCAAGA |
| siRNA 4443 | 4443 | CUUGGGUGUUCCUUAUCUG | 10797 | CAGAUAAGGAACACCCAAG |
| siRNA 4444 | 4444 | UUGGGUGUUCCUUAUCUG | 10798 | CCAGAUAAGGAACACCCAA |
| | | G | | |
| siRNA 4445 | 4445 | UGGGUGUUCCUUAUCUGG | 10799 | CCCAGAUAAGGAACACCCA |
| | | G | | |
| siRNA 4446 | 4446 | GGGUGUUCCUUAUCUGGG | 10800 | CCCCAGAUAAGGAACACCC |
| | | G | | |
| siRNA 4447 | 4447 | GGUGUUCCUUAUCUGGGG | 10801 | UCCCCAGAUAAGGAACACC |
| | | A | | |
| siRNA 4448 | 4448 | GUGUUCCUUAUCUGGGGA | 10802 | UUCCCCAGAUAAGGAACAC |
| | | A | | |
| siRNA 4449 | 4449 | UGUUCCUUAUCUGGGGAA | 10803 | CUUCCCCAGAUAAGGAACA |
| | | G | | |
| siRNA 4450 | 4450 | GUUCCUUAUCUGGGGAAGC | 10804 | GCUUCCCCAGAUAAGGAAC |
| siRNA 4451 | 4451 | UUCCUUAUCUGGGGAAGCA | 10805 | UGCUUCCCCAGAUAAGGAA |
| siRNA 4452 | 4452 | UCCUUAUCUGGGGAAGCAA | 10806 | UUGCUUCCCCAGAUAAGGA |
| siRNA 4453 | 4453 | CCUUAUCUGGGGAAGCAAA | 10807 | UUUGCUUCCCCAGAUAAGG |
| siRNA 4454 | 4454 | CUUAUCUGGGGAAGCAAAC | 10808 | GUUUGCUUCCCCAGAUAAG |
| siRNA 4455 | 4455 | UUAUCUGGGGAAGCAAAC | 10809 | UGUUUGCUUCCCCAGAUAA |
| | | A | | |
| siRNA 4456 | 4456 | UAUCUGGGGAAGCAAACA | 10810 | AUGUUUGCUUCCCCAGAUA |
| | | U | | |
| siRNA 4457 | 4457 | AUCUGGGGAAGCAAACAU | 10811 | AAUGUUUGCUUCCCCAGAU |
| | | U | | |
| siRNA 4458 | 4458 | UCUGGGGAAGCAAACAUU | 10812 | AAAUGUUUGCUUCCCCAGA |
| | | U | | |
| siRNA 4459 | 4459 | CUGGGGAAGCAAACAUUUC | 10813 | GAAAUGUUUGCUUCCCCAG |
| siRNA 4460 | 4460 | UGGGGAAGCAAACAUUUC | 10814 | UGAAAUGUUUGCUUCCCCA |
| | | A | | |
| siRNA 4461 | 4461 | GGGGAAGCAAACAUUUCAC | 10815 | GUGAAAUGUUUGCUUCCCC |
| siRNA 4462 | 4462 | GGGAAGCAAACAUUUCACU | 10816 | AGUGAAAUGUUUGCUUCCC |
| siRNA 4463 | 4463 | GGAAGCAAACAUUUCACUA | 10817 | UAGUGAAAUGUUUGCUUC |
| | | | | C |
| siRNA 4464 | 4464 | GAAGCAAACAUUUCACUAG | 10818 | CUAGUGAAAUGUUUGCUU |
| | | | | C |
| siRNA 4465 | 4465 | AAGCAAACAUUUCACUAGU | 10819 | ACUAGUGAAAUGUUUGCU |
| | | | | U |
| siRNA 4466 | 4466 | AGCAAACAUUUCACUAGUC | 10820 | GACUAGUGAAAUGUUUGC |
| | | | | U |
| siRNA 4467 | 4467 | GCAAACAUUUCACUAGUCU | 10821 | AGACUAGUGAAAUGUUUG |
| | | | | C |
| siRNA 4468 | 4468 | CAAACAUUUCACUAGUCUC | 10822 | GAGACUAGUGAAAUGUUU |
| | | | | G |
| siRNA 4469 | 4469 | AAACAUUUCACUAGUCUCU | 10823 | AGAGACUAGUGAAAUGUU |
| | | | | U |
| siRNA 4470 | 4470 | AACAUUUCACUAGUCUCUU | 10824 | AAGAGACUAGUGAAAUGU |
| | | | | U |
| siRNA 4471 | 4471 | ACAUUUCACUAGUCUCUUU | 10825 | AAAGAGACUAGUGAAAUG |
| | | | | U |
| siRNA 4472 | 4472 | CAUUUCACUAGUCUCUUUU | 10826 | AAAAGAGACUAGUGAAAU |
| | | | | G |
| siRNA 4473 | 4473 | AUUUCACUAGUCUCUUUUU | 10827 | AAAAAGAGACUAGUGAAA |
| | | | | U |
| siRNA 4474 | 4474 | UUUCACUAGUCUCUUUUUU | 10828 | AAAAAAGAGACUAGUGAA |
| | | | | A |
| siRNA 4475 | 4475 | UUCACUAGUCUCUUUUUUU | 10829 | AAAAAAAGAGACUAGUGA |
| | | | | A |
| siRNA 4476 | 4476 | UCACUAGUCUCUUUUUUUC | 10830 | GAAAAAAAGAGACUAGUG |
| | | | | A |
| siRNA 4477 | 4477 | CACUAGUCUCUUUUUUUCA | 10831 | UGAAAAAAAGAGACUAGU |
| | | | | G |
| siRNA 4478 | 4478 | ACUAGUCUCUUUUUUUCAU | 10832 | AUGAAAAAAAGAGACUAG |
| | | | | U |
| siRNA 4479 | 4479 | CUAGUCUCUUUUUUUCAUC | 10833 | GAUGAAAAAAAGAGACUA |
| | | | | G |
| siRNA 4480 | 4480 | UAGUCUCUUUUUUUCAUCC | 10834 | GGAUGAAAAAAAGAGACU |
| | | | | A |
| siRNA 4481 | 4481 | AGUCUCUUUUUUUCAUCCU | 10835 | AGGAUGAAAAAAAGAGAC |
| | | | | U |
| siRNA 4482 | 4482 | GUCUCUUUUUUUCAUCCUU | 10836 | AAGGAUGAAAAAAAGAGA |
| | | | | C |
| siRNA 4483 | 4483 | UCUCUUUUUUUCAUCCUUU | 10837 | AAAGGAUGAAAAAAAGAG |
| | | | | A |
| siRNA 4484 | 4484 | CUCUUUUUUUCAUCCUUUA | 10838 | UAAAGGAUGAAAAAAAGA |
| | | | | G |
| siRNA 4485 | 4485 | UCUUUUUUUCAUCCUUUAA | 10839 | UUAAAGGAUGAAAAAAAG |
| | | | | A |
| siRNA 4486 | 4486 | CUUUUUUUCAUCCUUUAAA | 10840 | UUUAAAGGAUGAAAAAAA |
| | | | | G |
| siRNA 4487 | 4487 | UUUUUUUCAUCCUUUAAA | 10841 | AUUUAAAGGAUGAAAAAA |
| | | U | | A |
| siRNA 4488 | 4488 | UUUUUUCAUCCUUUAAAU | 10842 | AAUUUAAAGGAUGAAAAA |
| | | U | | A |
| siRNA 4489 | 4489 | UUUUUCAUCCUUUAAAUU | 10843 | CAAUUUAAAGGAUGAAAA |
| | | G | | A |
| siRNA 4490 | 4490 | UUUUCAUCCUUUAAAUUG | 10844 | ACAAUUUAAAGGAUGAAA |
| | | U | | A |
| siRNA 4491 | 4491 | UUUCAUCCUUUAAAUUGU | 10845 | UACAAUUUAAAGGAUGAA |
| | | A | | A |
| siRNA 4492 | 4492 | UUCAUCCUUUAAAUUGUA | 10846 | UUACAAUUUAAAGGAUGA |
| | | A | | A |
| siRNA 4493 | 4493 | UCAUCCUUUAAAUUGUAA | 10847 | UUUACAAUUUAAAGGAUG |
| | | A | | A |
| siRNA 4494 | 4494 | CAUCCUUUAAAUUGUAAA | 10848 | AUUUACAAUUUAAAGGAU |
| | | U | | G |
| siRNA 4495 | 4495 | AUCCUUUAAAUUGUAAAU | 10849 | AAUUUACAAUUUAAAGGA |
| | | U | | U |
| siRNA 4496 | 4496 | UCCUUUAAAUUGUAAAUU | 10850 | UAAUUUACAAUUUAAAGG |
| | | A | | A |
| siRNA 4497 | 4497 | CCUUUAAAUUGUAAAUUA | 10851 | UUAAUUUACAAUUUAAAG |
| | | A | | G |
| siRNA 4498 | 4498 | CUUUAAAUUGUAAAUUAA | 10852 | CUUAAUUUACAAUUUAAA |
| | | G | | G |
| siRNA 4499 | 4499 | UUUAAAUUGUAAAUUAAG | 10853 | CCUUAAUUUACAAUUUAA |
| | | G | | A |
| siRNA 4500 | 4500 | UUAAAUUGUAAAUUAAGG | 10854 | UCCUUAAUUUACAAUUUA |
| | | A | | A |
| siRNA 4501 | 4501 | UAAAUUGUAAAUUAAGGA | 10855 | AUCCUUAAUUUACAAUUU |
| | | U | | A |
| siRNA 4502 | 4502 | AAAUUGUAAAUUAAGGAU | 10856 | AAUCCUUAAUUUACAAUU |
| | | U | | U |
| siRNA 4503 | 4503 | AAUUGUAAAUUAAGGAUU | 10857 | UAAUCCUUAAUUUACAAU |
| | | A | | U |
| siRNA 4504 | 4504 | AUUGUAAAUUAAGGAUUA | 10858 | GUAAUCCUUAAUUUACAA |
| | | C | | U |
| siRNA 4505 | 4505 | UUGUAAAUUAAGGAUUAC | 10859 | AGUAAUCCUUAAUUUACA |
| | | U | | A |
| siRNA 4506 | 4506 | UGUAAAUUAAGGAUUACU | 10860 | GAGUAAUCCUUAAUUUAC |
| | | C | | A |
| siRNA 4507 | 4507 | GUAAAUUAAGGAUUACUC | 10861 | UGAGUAAUCCUUAAUUUA |
| | | A | | C |
| siRNA 4508 | 4508 | UAAAUUAAGGAUUACUCA | 10862 | UUGAGUAAUCCUUAAUUU |
| | | A | | A |
| siRNA 4509 | 4509 | AAAUUAAGGAUUACUCAA | 10863 | CUUGAGUAAUCCUUAAUU |
| | | G | | U |
| siRNA 4510 | 4510 | AAUUAAGGAUUACUCAAG | 10864 | GCUUGAGUAAUCCUUAAU |
| | | C | | U |
| siRNA 4511 | 4511 | AUUAAGGAUUACUCAAGC | 10865 | AGCUUGAGUAAUCCUUAA |
| | | U | | U |
| siRNA 4512 | 4512 | UUAAGGAUUACUCAAGCUC | 10866 | GAGCUUGAGUAAUCCUUA |
| | | | | A |
| siRNA 4513 | 4513 | UAAGGAUUACUCAAGCUCA | 10867 | UGAGCUUGAGUAAUCCUU |
| | | | | A |
| siRNA 4514 | 4514 | AAGGAUUACUCAAGCUCAC | 10868 | GUGAGCUUGAGUAAUCCU |
| | | | | U |
| siRNA 4515 | 4515 | AGGAUUACUCAAGCUCACC | 10869 | GGUGAGCUUGAGUAAUCC |
| | | | | U |
| siRNA 4516 | 4516 | GGAUUACUCAAGCUCACCA | 10870 | UGGUGAGCUUGAGUAAUC |
| | | | | C |
| siRNA 4517 | 4517 | GAUUACUCAAGCUCACCAU | 10871 | AUGGUGAGCUUGAGUAAU |
| | | | | C |
| siRNA 4518 | 4518 | AUUACUCAAGCUCACCAUU | 10872 | AAUGGUGAGCUUGAGUAA |
| | | | | U |
| siRNA 4519 | 4519 | UUACUCAAGCUCACCAUUA | 10873 | UAAUGGUGAGCUUGAGUA |
| | | | | A |
| siRNA 4520 | 4520 | UACUCAAGCUCACCAUUAU | 10874 | AUAAUGGUGAGCUUGAGU |
| | | | | A |
| siRNA 4521 | 4521 | ACUCAAGCUCACCAUUAUU | 10875 | AAUAAUGGUGAGCUUGAG |
| | | | | U |
| siRNA 4522 | 4522 | CUCAAGCUCACCAUUAUUC | 10876 | GAAUAAUGGUGAGCUUGA |
| | | | | G |
| siRNA 4523 | 4523 | UCAAGCUCACCAUUAUUCA | 10877 | UGAAUAAUGGUGAGCUUG |
| | | | | A |
| siRNA 4524 | 4524 | CAAGCUCACCAUUAUUCAA | 10878 | UUGAAUAAUGGUGAGCUU |
| | | | | G |
| siRNA 4525 | 4525 | AAGCUCACCAUUAUUCAAG | 10879 | CUUGAAUAAUGGUGAGCU |
| | | | | U |
| siRNA 4526 | 4526 | AGCUCACCAUUAUUCAAGA | 10880 | UCUUGAAUAAUGGUGAGC |
| | | | | U |
| siRNA 4527 | 4527 | GCUCACCAUUAUUCAAGAU | 10881 | AUCUUGAAUAAUGGUGAG |
| | | | | C |
| siRNA 4528 | 4528 | CUCACCAUUAUUCAAGAUU | 10882 | AAUCUUGAAUAAUGGUGA |
| | | | | G |
| siRNA 4529 | 4529 | UCACCAUUAUUCAAGAUUG | 10883 | CAAUCUUGAAUAAUGGUG |
| | | | | A |
| siRNA 4530 | 4530 | CACCAUUAUUCAAGAUUGG | 10884 | CCAAUCUUGAAUAAUGGU |
| | | | | G |
| siRNA 4531 | 4531 | ACCAUUAUUCAAGAUUGG | 10885 | CCCAAUCUUGAAUAAUGGU |
| | | G | | |
| siRNA 4532 | 4532 | CCAUUAUUCAAGAUUGGG | 10886 | UCCCAAUCUUGAAUAAUGG |
| | | A | | |
| siRNA 4533 | 4533 | CAUUAUUCAAGAUUGGGA | 10887 | GUCCCAAUCUUGAAUAAUG |
| | | C | | |
| siRNA 4534 | 4534 | AUUAUUCAAGAUUGGGAC | 10888 | AGUCCCAAUCUUGAAUAAU |
| | | U | | |
| siRNA 4535 | 4535 | UUAUUCAAGAUUGGGACU | 10889 | GAGUCCCAAUCUUGAAUAA |
| | | C | | |
| siRNA 4536 | 4536 | UAUUCAAGAUUGGGACUC | 10890 | CGAGUCCCAAUCUUGAAUA |
| | | G | | |
| siRNA 4537 | 4537 | AUUCAAGAUUGGGACUCGC | 10891 | GCGAGUCCCAAUCUUGAAU |
| siRNA 4538 | 4538 | UUCAAGAUUGGGACUCGCU | 10892 | AGCGAGUCCCAAUCUUGAA |
| siRNA 4539 | 4539 | UCAAGAUUGGGACUCGCUU | 10893 | AAGCGAGUCCCAAUCUUGA |
| siRNA 4540 | 4540 | CAAGAUUGGGACUCGCUUC | 10894 | GAAGCGAGUCCCAAUCUUG |
| siRNA 4541 | 4541 | AAGAUUGGGACUCGCUUCC | 10895 | GGAAGCGAGUCCCAAUCUU |
| siRNA 4542 | 4542 | AGAUUGGGACUCGCUUCCC | 10896 | GGGAAGCGAGUCCCAAUCU |
| siRNA 4543 | 4543 | GAUUGGGACUCGCUUCCCA | 10897 | UGGGAAGCGAGUCCCAAUC |
| siRNA 4544 | 4544 | AUUGGGACUCGCUUCCCAG | 10898 | CUGGGAAGCGAGUCCCAAU |
| siRNA 4545 | 4545 | UUGGGACUCGCUUCCCAGU | 10899 | ACUGGGAAGCGAGUCCCAA |
| siRNA 4546 | 4546 | UGGGACUCGCUUCCCAGUC | 10900 | GACUGGGAAGCGAGUCCCA |
| siRNA 4547 | 4547 | GGGACUCGCUUCCCAGUCG | 10901 | CGACUGGGAAGCGAGUCCC |
| siRNA 4548 | 4548 | GGACUCGCUUCCCAGUCGA | 10902 | UCGACUGGGAAGCGAGUCC |
| siRNA 4549 | 4549 | GACUCGCUUCCCAGUCGAC | 10903 | GUCGACUGGGAAGCGAGUC |
| siRNA 4550 | 4550 | ACUCGCUUCCCAGUCGACA | 10904 | UGUCGACUGGGAAGCGAG |
| | | | | U |
| siRNA 4551 | 4551 | CUCGCUUCCCAGUCGACAC | 10905 | GUGUCGACUGGGAAGCGA |
| | | | | G |
| siRNA 4552 | 4552 | UCGCUUCCCAGUCGACACU | 10906 | AGUGUCGACUGGGAAGCG |
| | | | | A |
| siRNA 4553 | 4553 | CGCUUCCCAGUCGACACUC | 10907 | GAGUGUCGACUGGGAAGC |
| | | | | G |
| siRNA 4554 | 4554 | GCUUCCCAGUCGACACUCU | 10908 | AGAGUGUCGACUGGGAAG |
| | | | | C |
| siRNA 4555 | 4555 | CUUCCCAGUCGACACUCUG | 10909 | CAGAGUGUCGACUGGGAA |
| | | | | G |
| siRNA 4556 | 4556 | UUCCCAGUCGACACUCUGC | 10910 | GCAGAGUGUCGACUGGGA |
| | | | | A |
| siRNA 4557 | 4557 | UCCCAGUCGACACUCUGCC | 10911 | GGCAGAGUGUCGACUGGG |
| | | | | A |
| siRNA 4558 | 4558 | CCCAGUCGACACUCUGCCC | 10912 | GGGCAGAGUGUCGACUGG |
| | | | | G |
| siRNA 4559 | 4559 | CCAGUCGACACUCUGCCCU | 10913 | AGGGCAGAGUGUCGACUG |
| | | | | G |
| siRNA 4560 | 4560 | CAGUCGACACUCUGCCCUG | 10914 | CAGGGCAGAGUGUCGACUG |
| siRNA 4561 | 4561 | AGUCGACACUCUGCCCUGC | 10915 | GCAGGGCAGAGUGUCGACU |
| siRNA 4562 | 4562 | GUCGACACUCUGCCCUGCC | 10916 | GGCAGGGCAGAGUGUCGAC |
| siRNA 4563 | 4563 | UCGACACUCUGCCCUGCCU | 10917 | AGGCAGGGCAGAGUGUCG |
| | | | | A |
| siRNA 4564 | 4564 | CGACACUCUGCCCUGCCUG | 10918 | CAGGCAGGGCAGAGUGUCG |
| siRNA 4565 | 4565 | GACACUCUGCCCUGCCUGU | 10919 | ACAGGCAGGGCAGAGUGUC |
| siRNA 4566 | 4566 | ACACUCUGCCCUGCCUGUC | 10920 | GACAGGCAGGGCAGAGUG |
| | | | | U |
| siRNA 4567 | 4567 | CACUCUGCCCUGCCUGUCA | 10921 | UGACAGGCAGGGCAGAGU |
| | | | | G |
| siRNA 4568 | 4568 | ACUCUGCCCUGCCUGUCAU | 10922 | AUGACAGGCAGGGCAGAG |
| | | | | U |
| siRNA 4569 | 4569 | CUCUGCCCUGCCUGUCAUU | 10923 | AAUGACAGGCAGGGCAGA |
| | | | | G |
| siRNA 4570 | 4570 | UCUGCCCUGCCUGUCAUUG | 10924 | CAAUGACAGGCAGGGCAGA |
| siRNA 4571 | 4571 | CUGCCCUGCCUGUCAUUGC | 10925 | GCAAUGACAGGCAGGGCAG |
| siRNA 4572 | 4572 | UGCCCUGCCUGUCAUUGCU | 10926 | AGCAAUGACAGGCAGGGCA |
| siRNA 4573 | 4573 | GCCCUGCCUGUCAUUGCUG | 10927 | CAGCAAUGACAGGCAGGGC |
| siRNA 4574 | 4574 | CCCUGCCUGUCAUUGCUGC | 10928 | GCAGCAAUGACAGGCAGGG |
| siRNA 4575 | 4575 | CCUGCCUGUCAUUGCUGCA | 10929 | UGCAGCAAUGACAGGCAGG |
| siRNA 4576 | 4576 | CUGCCUGUCAUUGCUGCAA | 10930 | UUGCAGCAAUGACAGGCAG |
| siRNA 4577 | 4577 | UGCCUGUCAUUGCUGCAAA | 10931 | UUUGCAGCAAUGACAGGCA |
| siRNA 4578 | 4578 | GCCUGUCAUUGCUGCAAAG | 10932 | CUUUGCAGCAAUGACAGGC |
| siRNA 4579 | 4579 | CCUGUCAUUGCUGCAAAGA | 10933 | UCUUUGCAGCAAUGACAGG |
| siRNA 4580 | 4580 | CUGUCAUUGCUGCAAAGAG | 10934 | CUCUUUGCAGCAAUGACAG |
| siRNA 4581 | 4581 | UGUCAUUGCUGCAAAGAGC | 10935 | GCUCUUUGCAGCAAUGACA |
| siRNA 4582 | 4582 | GUCAUUGCUGCAAAGAGCU | 10936 | AGCUCUUUGCAGCAAUGAC |
| siRNA 4583 | 4583 | UCAUUGCUGCAAAGAGCUG | 10937 | CAGCUCUUUGCAGCAAUGA |
| siRNA 4584 | 4584 | CAUUGCUGCAAAGAGCUGC | 10938 | GCAGCUCUUUGCAGCAAUG |
| siRNA 4585 | 4585 | AUUGCUGCAAAGAGCUGCU | 10939 | AGCAGCUCUUUGCAGCAAU |
| siRNA 4586 | 4586 | UUGCUGCAAAGAGCUGCUG | 10940 | CAGCAGCUCUUUGCAGCAA |
| siRNA 4587 | 4587 | UGCUGCAAAGAGCUGCUGC | 10941 | GCAGCAGCUCUUUGCAGCA |
| siRNA 4588 | 4588 | GCUGCAAAGAGCUGCUGCU | 10942 | AGCAGCAGCUCUUUGCAGC |
| siRNA 4589 | 4589 | CUGCAAAGAGCUGCUGCUU | 10943 | AAGCAGCAGCUCUUUGCAG |
| siRNA 4590 | 4590 | UGCAAAGAGCUGCUGCUUU | 10944 | AAAGCAGCAGCUCUUUGCA |
| siRNA 4591 | 4591 | GCAAAGAGCUGCUGCUUUG | 10945 | CAAAGCAGCAGCUCUUUGC |
| siRNA 4592 | 4592 | CAAAGAGCUGCUGCUUUGC | 10946 | GCAAAGCAGCAGCUCUUUG |
| siRNA 4593 | 4593 | AAAGAGCUGCUGCUUUGCC | 10947 | GGCAAAGCAGCAGCUCUUU |
| siRNA 4594 | 4594 | AAGAGCUGCUGCUUUGCCA | 10948 | UGGCAAAGCAGCAGCUCUU |
| siRNA 4595 | 4595 | AGAGCUGCUGCUUUGCCAA | 10949 | UUGGCAAAGCAGCAGCUCU |
| siRNA 4596 | 4596 | GAGCUGCUGCUUUGCCAAC | 10950 | GUUGGCAAAGCAGCAGCUC |
| siRNA 4597 | 4597 | AGCUGCUGCUUUGCCAACC | 10951 | GGUUGGCAAAGCAGCAGCU |
| siRNA 4598 | 4598 | GCUGCUGCUUUGCCAACCU | 10952 | AGGUUGGCAAAGCAGCAGC |
| siRNA 4599 | 4599 | CUGCUGCUUUGCCAACCUA | 10953 | UAGGUUGGCAAAGCAGCA |
| | | | | G |
| siRNA 4600 | 4600 | UGCUGCUUUGCCAACCUAA | 10954 | UUAGGUUGGCAAAGCAGC |
| | | | | A |
| siRNA 4601 | 4601 | GCUGCUUUGCCAACCUAAG | 10955 | CUUAGGUUGGCAAAGCAGC |
| siRNA 4602 | 4602 | CUGCUUUGCCAACCUAAGC | 10956 | GCUUAGGUUGGCAAAGCA |
| | | | | G |
| siRNA 4603 | 4603 | UGCUUUGCCAACCUAAGCA | 10957 | UGCUUAGGUUGGCAAAGC |
| | | | | A |
| siRNA 4604 | 4604 | GCUUUGCCAACCUAAGCAA | 10958 | UUGCUUAGGUUGGCAAAG |
| | | | | C |
| siRNA 4605 | 4605 | CUUUGCCAACCUAAGCAAA | 10959 | UUUGCUUAGGUUGGCAAA |
| | | | | G |
| siRNA 4606 | 4606 | UUUGCCAACCUAAGCAAAG | 10960 | CUUUGCUUAGGUUGGCAA |
| | | | | A |
| siRNA 4607 | 4607 | UUGCCAACCUAAGCAAAGA | 10961 | UCUUUGCUUAGGUUGGCA |
| | | | | A |
| siRNA 4608 | 4608 | UGCCAACCUAAGCAAAGAA | 10962 | UUCUUUGCUUAGGUUGGC |
| | | | | A |
| siRNA 4609 | 4609 | GCCAACCUAAGCAAAGAAA | 10963 | UUUCUUUGCUUAGGUUGG |
| | | | | C |
| siRNA 4610 | 4610 | CCAACCUAAGCAAAGAAAA | 10964 | UUUUCUUUGCUUAGGUUG |
| | | | | G |
| siRNA 4611 | 4611 | CAACCUAAGCAAAGAAAAU | 10965 | AUUUUCUUUGCUUAGGUU |
| | | | | G |
| siRNA 4612 | 4612 | AACCUAAGCAAAGAAAAU | 10966 | UAUUUUCUUUGCUUAGGU |
| | | A | | U |
| siRNA 4613 | 4613 | ACCUAAGCAAAGAAAAUAC | 10967 | GUAUUUUCUUUGCUUAGG |
| | | | | U |
| siRNA 4614 | 4614 | CCUAAGCAAAGAAAAUACG | 10968 | CGUAUUUUCUUUGCUUAG |
| | | | | G |
| siRNA 4615 | 4615 | CUAAGCAAAGAAAAUACG | 10969 | CCGUAUUUUCUUUGCUUAG |
| | | G | | |
| siRNA 4616 | 4616 | UAAGCAAAGAAAAUACGG | 10970 | GCCGUAUUUUCUUUGCUUA |
| | | C | | |
| siRNA 4617 | 4617 | AAGCAAAGAAAAUACGGC | 10971 | AGCCGUAUUUUCUUUGCUU |
| | | U | | |
| siRNA 4618 | 4618 | AGCAAAGAAAAUACGGCU | 10972 | AAGCCGUAUUUUCUUUGCU |
| | | U | | |
| siRNA 4619 | 4619 | GCAAAGAAAAUACGGCUUC | 10973 | GAAGCCGUAUUUUCUUUGC |
| siRNA 4620 | 4620 | CAAAGAAAAUACGGCUUCU | 10974 | AGAAGCCGUAUUUUCUUU |
| | | | | G |
| siRNA 4621 | 4621 | AAAGAAAAUACGGCUUCUC | 10975 | GAGAAGCCGUAUUUUCUU |
| | | | | U |
| siRNA 4622 | 4622 | AAGAAAAUACGGCUUCUCU | 10976 | AGAGAAGCCGUAUUUUCU |
| | | | | U |
| siRNA 4623 | 4623 | AGAAAAUACGGCUUCUCUU | 10977 | AAGAGAAGCCGUAUUUUC |
| | | | | U |
| siRNA 4624 | 4624 | GAAAAUACGGCUUCUCUUG | 10978 | CAAGAGAAGCCGUAUUUUC |
| siRNA 4625 | 4625 | AAAAUACGGCUUCUCUUGC | 10979 | GCAAGAGAAGCCGUAUUU |
| | | | | U |
| siRNA 4626 | 4626 | AAAUACGGCUUCUCUUGCA | 10980 | UGCAAGAGAAGCCGUAUU |
| | | | | U |
| siRNA 4627 | 4627 | AAUACGGCUUCUCUUGCAU | 10981 | AUGCAAGAGAAGCCGUAU |
| | | | | U |
| siRNA 4628 | 4628 | AUACGGCUUCUCUUGCAUU | 10982 | AAUGCAAGAGAAGCCGUA |
| | | | | U |
| siRNA 4629 | 4629 | UACGGCUUCUCUUGCAUUA | 10983 | UAAUGCAAGAGAAGCCGU |
| | | | | A |
| siRNA 4630 | 4630 | ACGGCUUCUCUUGCAUUAU | 10984 | AUAAUGCAAGAGAAGCCG |
| | | | | U |
| siRNA 4631 | 4631 | CGGCUUCUCUUGCAUUAUU | 10985 | AAUAAUGCAAGAGAAGCC |
| | | | | G |
| siRNA 4632 | 4632 | GGCUUCUCUUGCAUUAUUU | 10986 | AAAUAAUGCAAGAGAAGC |
| | | | | C |
| siRNA 4633 | 4633 | GCUUCUCUUGCAUUAUUUU | 10987 | AAAAUAAUGCAAGAGAAG |
| | | | | C |
| siRNA 4634 | 4634 | CUUCUCUUGCAUUAUUUUC | 10988 | GAAAAUAAUGCAAGAGAA |
| | | | | G |
| siRNA 4635 | 4635 | UUCUCUUGCAUUAUUUUCC | 10989 | GGAAAAUAAUGCAAGAGA |
| | | | | A |
| siRNA 4636 | 4636 | UCUCUUGCAUUAUUUUCCC | 10990 | GGGAAAAUAAUGCAAGAG |
| | | | | A |
| siRNA 4637 | 4637 | CUCUUGCAUUAUUUUCCCU | 10991 | AGGGAAAAUAAUGCAAGA |
| | | | | G |
| siRNA 4638 | 4638 | UCUUGCAUUAUUUUCCCUU | 10992 | AAGGGAAAAUAAUGCAAG |
| | | | | A |
| siRNA 4639 | 4639 | CUUGCAUUAUUUUCCCUUU | 10993 | AAAGGGAAAAUAAUGCAA |
| | | | | G |
| siRNA 4640 | 4640 | UUGCAUUAUUUUCCCUUUU | 10994 | AAAAGGGAAAAUAAUGCA |
| | | | | A |
| siRNA 4641 | 4641 | UGCAUUAUUUUCCCUUUUG | 10995 | CAAAAGGGAAAAUAAUGC |
| | | | | A |
| siRNA 4642 | 4642 | GCAUUAUUUUCCCUUUUGG | 10996 | CCAAAAGGGAAAAUAAUG |
| | | | | C |
| siRNA 4643 | 4643 | CAUUAUUUUCCCUUUUGGU | 10997 | ACCAAAAGGGAAAAUAAU |
| | | | | G |
| siRNA 4644 | 4644 | AUUAUUUUCCCUUUUGGU | 10998 | AACCAAAAGGGAAAAUAA |
| | | U | | U |
| siRNA 4645 | 4645 | UUAUUUUCCCUUUUGGUU | 10999 | CAACCAAAAGGGAAAAUA |
| | | G | | A |
| siRNA 4646 | 4646 | UAUUUUCCCUUUUGGUUG | 11000 | CCAACCAAAAGGGAAAAUA |
| | | G | | |
| siRNA 4647 | 4647 | AUUUUCCCUUUUGGUUGG | 11001 | ACCAACCAAAAGGGAAAAU |
| | | U | | |
| siRNA 4648 | 4648 | UUUUCCCUUUUGGUUGGU | 11002 | AACCAACCAAAAGGGAAAA |
| | | U | | |
| siRNA 4649 | 4649 | UUUCCCUUUUGGUUGGUU | 11003 | AAACCAACCAAAAGGGAAA |
| | | U | | |
| siRNA 4650 | 4650 | UUCCCUUUUGGUUGGUUU | 11004 | CAAACCAACCAAAAGGGAA |
| | | G | | |
| siRNA 4651 | 4651 | UCCCUUUUGGUUGGUUUG | 11005 | ACAAACCAACCAAAAGGGA |
| | | U | | |
| siRNA 4652 | 4652 | CCCUUUUGGUUGGUUUGU | 11006 | AACAAACCAACCAAAAGGG |
| | | U | | |
| siRNA 4653 | 4653 | CCUUUUGGUUGGUUUGUU | 11007 | AAACAAACCAACCAAAAGG |
| | | U | | |
| siRNA 4654 | 4654 | CUUUUGGUUGGUUUGUUU | 11008 | AAAACAAACCAACCAAAAG |
| | | U | | |
| siRNA 4655 | 4655 | UUUUGGUUGGUUUGUUUU | 11009 | GAAAACAAACCAACCAAAA |
| | | C | | |
| siRNA 4656 | 4656 | UUUGGUUGGUUUGUUUUC | 11010 | AGAAAACAAACCAACCAAA |
| | | U | | |
| siRNA 4657 | 4657 | UUGGUUGGUUUGUUUUCU | 11011 | UAGAAAACAAACCAACCAA |
| | | A | | |
| siRNA 4658 | 4658 | UGGUUGGUUUGUUUUCUA | 11012 | CUAGAAAACAAACCAACCA |
| | | G | | |
| siRNA 4659 | 4659 | GGUUGGUUUGUUUUCUAG | 11013 | UCUAGAAAACAAACCAACC |
| | | A | | |
| siRNA 4660 | 4660 | GUUGGUUUGUUUUCUAGA | 11014 | UUCUAGAAAACAAACCAAC |
| | | A | | |
| siRNA 4661 | 4661 | UUGGUUUGUUUUCUAGAA | 11015 | CUUCUAGAAAACAAACCAA |
| | | G | | |
| siRNA 4662 | 4662 | UGGUUUGUUUUCUAGAAG | 11016 | ACUUCUAGAAAACAAACCA |
| | | U | | |
| siRNA 4663 | 4663 | GGUUUGUUUUCUAGAAGU | 11017 | UACUUCUAGAAAACAAACC |
| | | A | | |
| siRNA 4664 | 4664 | GUUUGUUUUCUAGAAGUA | 11018 | GUACUUCUAGAAAACAAAC |
| | | C | | |
| siRNA 4665 | 4665 | UUUGUUUUCUAGAAGUAC | 11019 | CGUACUUCUAGAAAACAAA |
| | | G | | |
| siRNA 4666 | 4666 | UUGUUUUCUAGAAGUACG | 11020 | ACGUACUUCUAGAAAACAA |
| | | U | | |
| siRNA 4667 | 4667 | UGUUUUCUAGAAGUACGU | 11021 | AACGUACUUCUAGAAAACA |
| | | U | | |
| siRNA 4668 | 4668 | GUUUUCUAGAAGUACGUU | 11022 | GAACGUACUUCUAGAAAAC |
| | | C | | |
| siRNA 4669 | 4669 | UUUUCUAGAAGUACGUUC | 11023 | UGAACGUACUUCUAGAAA |
| | | A | | A |
| siRNA 4670 | 4670 | UUUCUAGAAGUACGUUCA | 11024 | CUGAACGUACUUCUAGAAA |
| | | G | | |
| siRNA 4671 | 4671 | UUCUAGAAGUACGUUCAG | 11025 | UCUGAACGUACUUCUAGAA |
| | | A | | |
| siRNA 4672 | 4672 | UCUAGAAGUACGUUCAGA | 11026 | AUCUGAACGUACUUCUAGA |
| | | U | | |
| siRNA 4673 | 4673 | CUAGAAGUACGUUCAGAU | 11027 | CAUCUGAACGUACUUCUAG |
| | | G | | |
| siRNA 4674 | 4674 | UAGAAGUACGUUCAGAUG | 11028 | GCAUCUGAACGUACUUCUA |
| | | C | | |
| siRNA 4675 | 4675 | AGAAGUACGUUCAGAUGC | 11029 | AGCAUCUGAACGUACUUCU |
| | | U | | |
| siRNA 4676 | 4676 | GAAGUACGUUCAGAUGCU | 11030 | AAGCAUCUGAACGUACUUC |
| | | U | | |
| siRNA 4677 | 4677 | AAGUACGUUCAGAUGCUU | 11031 | AAAGCAUCUGAACGUACUU |
| | | U | | |
| siRNA 4678 | 4678 | AGUACGUUCAGAUGCUUU | 11032 | CAAAGCAUCUGAACGUACU |
| | | G | | |
| siRNA 4679 | 4679 | GUACGUUCAGAUGCUUUG | 11033 | CCAAAGCAUCUGAACGUAC |
| | | G | | |
| siRNA 4680 | 4680 | UACGUUCAGAUGCUUUGG | 11034 | CCCAAAGCAUCUGAACGUA |
| | | G | | |
| siRNA 4681 | 4681 | ACGUUCAGAUGCUUUGGG | 11035 | CCCCAAAGCAUCUGAACGU |
| | | G | | |
| siRNA 4682 | 4682 | CGUUCAGAUGCUUUGGGG | 11036 | UCCCCAAAGCAUCUGAACG |
| | | A | | |
| siRNA 4683 | 4683 | GUUCAGAUGCUUUGGGGA | 11037 | UUCCCCAAAGCAUCUGAAC |
| | | A | | |
| siRNA 4684 | 4684 | UUCAGAUGCUUUGGGGAA | 11038 | AUUCCCCAAAGCAUCUGAA |
| | | U | | |
| siRNA 4685 | 4685 | UCAGAUGCUUUGGGGAAU | 11039 | CAUUCCCCAAAGCAUCUGA |
| | | G | | |
| siRNA 4686 | 4686 | CAGAUGCUUUGGGGAAUG | 11040 | GCAUUCCCCAAAGCAUCUG |
| | | C | | |
| siRNA 4687 | 4687 | AGAUGCUUUGGGGAAUGC | 11041 | UGCAUUCCCCAAAGCAUCU |
| | | A | | |
| siRNA 4688 | 4688 | GAUGCUUUGGGGAAUGCA | 11042 | UUGCAUUCCCCAAAGCAUC |
| | | A | | |
| siRNA 4689 | 4689 | AUGCUUUGGGGAAUGCAA | 11043 | AUUGCAUUCCCCAAAGCAU |
| | | U | | |
| siRNA 4690 | 4690 | UGCUUUGGGGAAUGCAAU | 11044 | CAUUGCAUUCCCCAAAGCA |
| | | G | | |
| siRNA 4691 | 4691 | GCUUUGGGGAAUGCAAUG | 11045 | ACAUUGCAUUCCCCAAAGC |
| | | U | | |
| siRNA 4692 | 4692 | CUUUGGGGAAUGCAAUGU | 11046 | UACAUUGCAUUCCCCAAAG |
| | | A | | |
| siRNA 4693 | 4693 | UUUGGGGAAUGCAAUGUA | 11047 | AUACAUUGCAUUCCCCAAA |
| | | U | | |
| siRNA 4694 | 4694 | UUGGGGAAUGCAAUGUAU | 11048 | CAUACAUUGCAUUCCCCAA |
| | | G | | |
| siRNA 4695 | 4695 | UGGGGAAUGCAAUGUAUG | 11049 | UCAUACAUUGCAUUCCCCA |
| | | A | | |
| siRNA 4696 | 4696 | GGGGAAUGCAAUGUAUGA | 11050 | AUCAUACAUUGCAUUCCCC |
| | | U | | |
| siRNA 4697 | 4697 | GGGAAUGCAAUGUAUGAU | 11051 | AAUCAUACAUUGCAUUCCC |
| | | U | | |
| siRNA 4698 | 4698 | GGAAUGCAAUGUAUGAUU | 11052 | AAAUCAUACAUUGCAUUCC |
| | | U | | |
| siRNA 4699 | 4699 | GAAUGCAAUGUAUGAUUU | 11053 | CAAAUCAUACAUUGCAUUC |
| | | G | | |
| siRNA 4700 | 4700 | AAUGCAAUGUAUGAUUUG | 11054 | GCAAAUCAUACAUUGCAUU |
| | | C | | |
| siRNA 4701 | 4701 | AUGCAAUGUAUGAUUUGC | 11055 | AGCAAAUCAUACAUUGCAU |
| | | U | | |
| siRNA 4702 | 4702 | UGCAAUGUAUGAUUUGCU | 11056 | UAGCAAAUCAUACAUUGCA |
| | | A | | |
| siRNA 4703 | 4703 | GCAAUGUAUGAUUUGCUA | 11057 | CUAGCAAAUCAUACAUUGC |
| | | G | | |
| siRNA 4704 | 4704 | CAAUGUAUGAUUUGCUAG | 11058 | GCUAGCAAAUCAUACAUUG |
| | | C | | |
| siRNA 4705 | 4705 | AAUGUAUGAUUUGCUAGC | 11059 | AGCUAGCAAAUCAUACAUU |
| | | U | | |
| siRNA 4706 | 4706 | AUGUAUGAUUUGCUAGCU | 11060 | GAGCUAGCAAAUCAUACAU |
| | | C | | |
| siRNA 4707 | 4707 | UGUAUGAUUUGCUAGCUC | 11061 | AGAGCUAGCAAAUCAUACA |
| | | U | | |
| siRNA 4708 | 4708 | GUAUGAUUUGCUAGCUCUC | 11062 | GAGAGCUAGCAAAUCAUAC |
| siRNA 4709 | 4709 | UAUGAUUUGCUAGCUCUCU | 11063 | AGAGAGCUAGCAAAUCAU |
| | | | | A |
| siRNA 4710 | 4710 | AUGAUUUGCUAGCUCUCUC | 11064 | GAGAGAGCUAGCAAAUCA |
| | | | | U |
| siRNA 4711 | 4711 | UGAUUUGCUAGCUCUCUCA | 11065 | UGAGAGAGCUAGCAAAUC |
| | | | | A |
| siRNA 4712 | 4712 | GAUUUGCUAGCUCUCUCAC | 11066 | GUGAGAGAGCUAGCAAAU |
| | | | | C |
| siRNA 4713 | 4713 | AUUUGCUAGCUCUCUCACC | 11067 | GGUGAGAGAGCUAGCAAA |
| | | | | U |
| siRNA 4714 | 4714 | UUUGCUAGCUCUCUCACCA | 11068 | UGGUGAGAGAGCUAGCAA |
| | | | | A |
| siRNA 4715 | 4715 | UUGCUAGCUCUCUCACCAC | 11069 | GUGGUGAGAGAGCUAGCA |
| | | | | A |
| siRNA 4716 | 4716 | UGCUAGCUCUCUCACCACU | 11070 | AGUGGUGAGAGAGCUAGC |
| | | | | A |
| siRNA 4717 | 4717 | GCUAGCUCUCUCACCACUU | 11071 | AAGUGGUGAGAGAGCUAG |
| | | | | C |
| siRNA 4718 | 4718 | CUAGCUCUCUCACCACUUA | 11072 | UAAGUGGUGAGAGAGCUA |
| | | | | G |
| siRNA 4719 | 4719 | UAGCUCUCUCACCACUUAA | 11073 | UUAAGUGGUGAGAGAGCU |
| | | | | A |
| siRNA 4720 | 4720 | AGCUCUCUCACCACUUAAC | 11074 | GUUAAGUGGUGAGAGAGC |
| | | | | U |
| siRNA 4721 | 4721 | GCUCUCUCACCACUUAACU | 11075 | AGUUAAGUGGUGAGAGAG |
| | | | | C |
| siRNA 4722 | 4722 | CUCUCUCACCACUUAACUC | 11076 | GAGUUAAGUGGUGAGAGA |
| | | | | G |
| siRNA 4723 | 4723 | UCUCUCACCACUUAACUCA | 11077 | UGAGUUAAGUGGUGAGAG |
| | | | | A |
| siRNA 4724 | 4724 | CUCUCACCACUUAACUCAC | 11078 | GUGAGUUAAGUGGUGAGA |
| | | | | G |
| siRNA 4725 | 4725 | UCUCACCACUUAACUCACU | 11079 | AGUGAGUUAAGUGGUGAG |
| | | | | A |
| siRNA 4726 | 4726 | CUCACCACUUAACUCACUG | 11080 | CAGUGAGUUAAGUGGUGA |
| | | | | G |
| siRNA 4727 | 4727 | UCACCACUUAACUCACUGU | 11081 | ACAGUGAGUUAAGUGGUG |
| | | | | A |
| siRNA 4728 | 4728 | CACCACUUAACUCACUGUG | 11082 | CACAGUGAGUUAAGUGGU |
| | | | | G |
| siRNA 4729 | 4729 | ACCACUUAACUCACUGUGA | 11083 | UCACAGUGAGUUAAGUGG |
| | | | | U |
| siRNA 4730 | 4730 | CCACUUAACUCACUGUGAG | 11084 | CUCACAGUGAGUUAAGUG |
| | | | | G |
| siRNA 4731 | 4731 | CACUUAACUCACUGUGAGG | 11085 | CCUCACAGUGAGUUAAGUG |
| siRNA 4732 | 4732 | ACUUAACUCACUGUGAGGA | 11086 | UCCUCACAGUGAGUUAAGU |
| siRNA 4733 | 4733 | CUUAACUCACUGUGAGGAU | 11087 | AUCCUCACAGUGAGUUAAG |
| siRNA 4734 | 4734 | UUAACUCACUGUGAGGAU | 11088 | UAUCCUCACAGUGAGUUAA |
| | | A | | |
| siRNA 4735 | 4735 | UAACUCACUGUGAGGAUA | 11089 | UUAUCCUCACAGUGAGUUA |
| | | A | | |
| siRNA 4736 | 4736 | AACUCACUGUGAGGAUAA | 11090 | UUUAUCCUCACAGUGAGUU |
| | | A | | |
| siRNA 4737 | 4737 | ACUCACUGUGAGGAUAAA | 11091 | AUUUAUCCUCACAGUGAGU |
| | | U | | |
| siRNA 4738 | 4738 | CUCACUGUGAGGAUAAAU | 11092 | UAUUUAUCCUCACAGUGAG |
| | | A | | |
| siRNA 4739 | 4739 | UCACUGUGAGGAUAAAUA | 11093 | AUAUUUAUCCUCACAGUGA |
| | | U | | |
| siRNA 4740 | 4740 | CACUGUGAGGAUAAAUAU | 11094 | CAUAUUUAUCCUCACAGUG |
| | | G | | |
| siRNA 4741 | 4741 | ACUGUGAGGAUAAAUAUG | 11095 | GCAUAUUUAUCCUCACAGU |
| | | C | | |
| siRNA 4742 | 4742 | CUGUGAGGAUAAAUAUGC | 11096 | UGCAUAUUUAUCCUCACAG |
| | | A | | |
| siRNA 4743 | 4743 | UGUGAGGAUAAAUAUGCA | 11097 | AUGCAUAUUUAUCCUCACA |
| | | U | | |
| siRNA 4744 | 4744 | GUGAGGAUAAAUAUGCAU | 11098 | CAUGCAUAUUUAUCCUCAC |
| | | G | | |
| siRNA 4745 | 4745 | UGAGGAUAAAUAUGCAUG | 11099 | GCAUGCAUAUUUAUCCUCA |
| | | C | | |
| siRNA 4746 | 4746 | GAGGAUAAAUAUGCAUGC | 11100 | AGCAUGCAUAUUUAUCCUC |
| | | U | | |
| siRNA 4747 | 4747 | AGGAUAAAUAUGCAUGCU | 11101 | AAGCAUGCAUAUUUAUCCU |
| | | U | | |
| siRNA 4748 | 4748 | GGAUAAAUAUGCAUGCUU | 11102 | AAAGCAUGCAUAUUUAUCC |
| | | U | | |
| siRNA 4749 | 4749 | GAUAAAUAUGCAUGCUUU | 11103 | AAAAGCAUGCAUAUUUAU |
| | | U | | C |
| siRNA 4750 | 4750 | AUAAAUAUGCAUGCUUUU | 11104 | AAAAAGCAUGCAUAUUUA |
| | | U | | U |
| siRNA 4751 | 4751 | UAAAUAUGCAUGCUUUUU | 11105 | CAAAAAGCAUGCAUAUUU |
| | | G | | A |
| siRNA 4752 | 4752 | AAAUAUGCAUGCUUUUUG | 11106 | ACAAAAAGCAUGCAUAUU |
| | | U | | U |
| siRNA 4753 | 4753 | AAUAUGCAUGCUUUUUGU | 11107 | UACAAAAAGCAUGCAUAU |
| | | A | | U |
| siRNA 4754 | 4754 | AUAUGCAUGCUUUUUGUA | 11108 | UUACAAAAAGCAUGCAUA |
| | | A | | U |
| siRNA 4755 | 4755 | UAUGCAUGCUUUUUGUAA | 11109 | AUUACAAAAAGCAUGCAU |
| | | U | | A |
| siRNA 4756 | 4756 | AUGCAUGCUUUUUGUAAU | 11110 | AAUUACAAAAAGCAUGCA |
| | | U | | U |
| siRNA 4757 | 4757 | UGCAUGCUUUUUGUAAUU | 11111 | UAAUUACAAAAAGCAUGC |
| | | A | | A |
| siRNA 4758 | 4758 | GCAUGCUUUUUGUAAUUA | 11112 | UUAAUUACAAAAAGCAUG |
| | | A | | C |
| siRNA 4759 | 4759 | CAUGCUUUUUGUAAUUAA | 11113 | GUUAAUUACAAAAAGCAU |
| | | C | | G |
| siRNA 4760 | 4760 | AUGCUUUUUGUAAUUAAC | 11114 | AGUUAAUUACAAAAAGCA |
| | | U | | U |
| siRNA 4761 | 4761 | UGCUUUUUGUAAUUAACU | 11115 | CAGUUAAUUACAAAAAGC |
| | | G | | A |
| siRNA 4762 | 4762 | GCUUUUUGUAAUUAACUG | 11116 | CCAGUUAAUUACAAAAAGC |
| | | G | | |
| siRNA 4763 | 4763 | CUUUUUGUAAUUAACUGG | 11117 | ACCAGUUAAUUACAAAAA |
| | | U | | G |
| siRNA 4764 | 4764 | UUUUUGUAAUUAACUGGU | 11118 | CACCAGUUAAUUACAAAAA |
| | | G | | |
| siRNA 4765 | 4765 | UUUUGUAAUUAACUGGUG | 11119 | GCACCAGUUAAUUACAAAA |
| | | C | | |
| siRNA 4766 | 4766 | UUUGUAAUUAACUGGUGC | 11120 | AGCACCAGUUAAUUACAAA |
| | | U | | |
| siRNA 4767 | 4767 | UUGUAAUUAACUGGUGCU | 11121 | AAGCACCAGUUAAUUACAA |
| | | U | | |
| siRNA 4768 | 4768 | UGUAAUUAACUGGUGCUU | 11122 | AAAGCACCAGUUAAUUACA |
| | | U | | |
| siRNA 4769 | 4769 | GUAAUUAACUGGUGCUUU | 11123 | CAAAGCACCAGUUAAUUAC |
| | | G | | |
| siRNA 4770 | 4770 | UAAUUAACUGGUGCUUUG | 11124 | UCAAAGCACCAGUUAAUUA |
| | | A | | |
| siRNA 4771 | 4771 | AAUUAACUGGUGCUUUGA | 11125 | UUCAAAGCACCAGUUAAUU |
| | | A | | |
| siRNA 4772 | 4772 | AUUAACUGGUGCUUUGAA | 11126 | UUUCAAAGCACCAGUUAAU |
| | | A | | |
| siRNA 4773 | 4773 | UUAACUGGUGCUUUGAAA | 11127 | UUUUCAAAGCACCAGUUAA |
| | | A | | |
| siRNA 4774 | 4774 | UAACUGGUGCUUUGAAAA | 11128 | AUUUUCAAAGCACCAGUUA |
| | | U | | |
| siRNA 4775 | 4775 | AACUGGUGCUUUGAAAAU | 11129 | GAUUUUCAAAGCACCAGUU |
| | | C | | |
| siRNA 4776 | 4776 | ACUGGUGCUUUGAAAAUC | 11130 | AGAUUUUCAAAGCACCAGU |
| | | U | | |
| siRNA 4777 | 4777 | CUGGUGCUUUGAAAAUCU | 11131 | AAGAUUUUCAAAGCACCAG |
| | | U | | |
| siRNA 4778 | 4778 | UGGUGCUUUGAAAAUCUU | 11132 | AAAGAUUUUCAAAGCACCA |
| | | U | | |
| siRNA 4779 | 4779 | GGUGCUUUGAAAAUCUUU | 11133 | AAAAGAUUUUCAAAGCACC |
| | | U | | |
| siRNA 4780 | 4780 | GUGCUUUGAAAAUCUUUU | 11134 | AAAAAGAUUUUCAAAGCA |
| | | U | | C |
| siRNA 4781 | 4781 | UGCUUUGAAAAUCUUUUU | 11135 | AAAAAAGAUUUUCAAAGC |
| | | U | | A |
| siRNA 4782 | 4782 | GCUUUGAAAAUCUUUUUU | 11136 | UAAAAAAGAUUUUCAAAG |
| | | A | | C |
| siRNA 4783 | 4783 | CUUUGAAAAUCUUUUUUA | 11137 | UUAAAAAAGAUUUUCAAA |
| | | A | | G |
| siRNA 4784 | 4784 | UUUGAAAAUCUUUUUUAA | 11138 | CUUAAAAAAGAUUUUCAA |
| | | G | | A |
| siRNA 4785 | 4785 | UUGAAAAUCUUUUUUAAG | 11139 | CCUUAAAAAAGAUUUUCA |
| | | G | | A |
| siRNA 4786 | 4786 | UGAAAAUCUUUUUUAAGG | 11140 | CCCUUAAAAAAGAUUUUCA |
| | | G | | |
| siRNA 4787 | 4787 | GAAAAUCUUUUUUAAGGG | 11141 | UCCCUUAAAAAAGAUUUUC |
| | | A | | |
| siRNA 4788 | 4788 | AAAAUCUUUUUUAAGGGA | 11142 | CUCCCUUAAAAAAGAUUUU |
| | | G | | |
| siRNA 4789 | 4789 | AAAUCUUUUUUAAGGGAG | 11143 | UCUCCCUUAAAAAAGAUUU |
| | | A | | |
| siRNA 4790 | 4790 | AAUCUUUUUUAAGGGAGA | 11144 | UUCUCCCUUAAAAAAGAUU |
| | | A | | |
| siRNA 4791 | 4791 | AUCUUUUUUAAGGGAGAA | 11145 | UUUCUCCCUUAAAAAAGAU |
| | | A | | |
| siRNA 4792 | 4792 | UCUUUUUUAAGGGAGAAA | 11146 | UUUUCUCCCUUAAAAAAGA |
| | | A | | |
| siRNA 4793 | 4793 | CUUUUUUAAGGGAGAAAA | 11147 | UUUUUCUCCCUUAAAAAAG |
| | | A | | |
| siRNA 4794 | 4794 | UUUUUUAAGGGAGAAAAA | 11148 | AUUUUUCUCCCUUAAAAAA |
| | | U | | |
| siRNA 4795 | 4795 | UUUUUAAGGGAGAAAAAU | 11149 | GAUUUUUCUCCCUUAAAAA |
| | | C | | |
| siRNA 4796 | 4796 | UUUUAAGGGAGAAAAAUC | 11150 | AGAUUUUUCUCCCUUAAAA |
| | | U | | |
| siRNA 4797 | 4797 | UUUAAGGGAGAAAAAUCU | 11151 | GAGAUUUUUCUCCCUUAAA |
| | | C | | |
| siRNA 4798 | 4798 | UUAAGGGAGAAAAAUCUC | 11152 | UGAGAUUUUUCUCCCUUAA |
| | | A | | |
| siRNA 4799 | 4799 | UAAGGGAGAAAAAUCUCA | 11153 | UUGAGAUUUUUCUCCCUUA |
| | | A | | |
| siRNA 4800 | 4800 | AAGGGAGAAAAAUCUCAA | 11154 | GUUGAGAUUUUUCUCCCUU |
| | | C | | |
| siRNA 4801 | 4801 | AGGGAGAAAAAUCUCAACC | 11155 | GGUUGAGAUUUUUCUCCCU |
| siRNA 4802 | 4802 | GGGAGAAAAAUCUCAACCA | 11156 | UGGUUGAGAUUUUUCUCCC |
| siRNA 4803 | 4803 | GGAGAAAAAUCUCAACCAA | 11157 | UUGGUUGAGAUUUUUCUC |
| | | | | C |
| siRNA 4804 | 4804 | GAGAAAAAUCUCAACCAAA | 11158 | UUUGGUUGAGAUUUUUCU |
| | | | | C |
| siRNA 4805 | 4805 | AGAAAAAUCUCAACCAAAG | 11159 | CUUUGGUUGAGAUUUUUC |
| | | | | U |
| siRNA 4806 | 4806 | GAAAAAUCUCAACCAAAGU | 11160 | ACUUUGGUUGAGAUUUUU |
| | | | | C |
| siRNA 4807 | 4807 | AAAAAUCUCAACCAAAGUU | 11161 | AACUUUGGUUGAGAUUUU |
| | | | | U |
| siRNA 4808 | 4808 | AAAAUCUCAACCAAAGUUA | 11162 | UAACUUUGGUUGAGAUUU |
| | | | | U |
| siRNA 4809 | 4809 | AAAUCUCAACCAAAGUUAU | 11163 | AUAACUUUGGUUGAGAUU |
| | | | | U |
| siRNA 4810 | 4810 | AAUCUCAACCAAAGUUAUG | 11164 | CAUAACUUUGGUUGAGAU |
| | | | | U |
| siRNA 4811 | 4811 | AUCUCAACCAAAGUUAUGC | 11165 | GCAUAACUUUGGUUGAGA |
| | | | | U |
| siRNA 4812 | 4812 | UCUCAACCAAAGUUAUGCU | 11166 | AGCAUAACUUUGGUUGAG |
| | | | | A |
| siRNA 4813 | 4813 | CUCAACCAAAGUUAUGCUC | 11167 | GAGCAUAACUUUGGUUGA |
| | | | | G |
| siRNA 4814 | 4814 | UCAACCAAAGUUAUGCUCA | 11168 | UGAGCAUAACUUUGGUUG |
| | | | | A |
| siRNA 4815 | 4815 | CAACCAAAGUUAUGCUCAU | 11169 | AUGAGCAUAACUUUGGUU |
| | | | | G |
| siRNA 4816 | 4816 | AACCAAAGUUAUGCUCAUC | 11170 | GAUGAGCAUAACUUUGGU |
| | | | | U |
| siRNA 4817 | 4817 | ACCAAAGUUAUGCUCAUCC | 11171 | GGAUGAGCAUAACUUUGG |
| | | | | U |
| siRNA 4818 | 4818 | CCAAAGUUAUGCUCAUCCA | 11172 | UGGAUGAGCAUAACUUUG |
| | | | | G |
| siRNA 4819 | 4819 | CAAAGUUAUGCUCAUCCAG | 11173 | CUGGAUGAGCAUAACUUU |
| | | | | G |
| siRNA 4820 | 4820 | AAAGUUAUGCUCAUCCAGA | 11174 | UCUGGAUGAGCAUAACUU |
| | | | | U |
| siRNA 4821 | 4821 | AAGUUAUGCUCAUCCAGAC | 11175 | GUCUGGAUGAGCAUAACU |
| | | | | U |
| siRNA 4822 | 4822 | AGUUAUGCUCAUCCAGACA | 11176 | UGUCUGGAUGAGCAUAAC |
| | | | | U |
| siRNA 4823 | 4823 | GUUAUGCUCAUCCAGACAA | 11177 | UUGUCUGGAUGAGCAUAA |
| | | | | C |
| siRNA 4824 | 4824 | UUAUGCUCAUCCAGACAAG | 11178 | CUUGUCUGGAUGAGCAUA |
| | | | | A |
| siRNA 4825 | 4825 | UAUGCUCAUCCAGACAAGC | 11179 | GCUUGUCUGGAUGAGCAU |
| | | | | A |
| siRNA 4826 | 4826 | AUGCUCAUCCAGACAAGCU | 11180 | AGCUUGUCUGGAUGAGCA |
| | | | | U |
| siRNA 4827 | 4827 | UGCUCAUCCAGACAAGCUG | 11181 | CAGCUUGUCUGGAUGAGCA |
| siRNA 4828 | 4828 | GCUCAUCCAGACAAGCUGA | 11182 | UCAGCUUGUCUGGAUGAGC |
| siRNA 4829 | 4829 | CUCAUCCAGACAAGCUGAC | 11183 | GUCAGCUUGUCUGGAUGA |
| | | | | G |
| siRNA 4830 | 4830 | UCAUCCAGACAAGCUGACC | 11184 | GGUCAGCUUGUCUGGAUG |
| | | | | A |
| siRNA 4831 | 4831 | CAUCCAGACAAGCUGACCU | 11185 | AGGUCAGCUUGUCUGGAU |
| | | | | G |
| siRNA 4832 | 4832 | AUCCAGACAAGCUGACCUU | 11186 | AAGGUCAGCUUGUCUGGA |
| | | | | U |
| siRNA 4833 | 4833 | UCCAGACAAGCUGACCUUU | 11187 | AAAGGUCAGCUUGUCUGG |
| | | | | A |
| siRNA 4834 | 4834 | CCAGACAAGCUGACCUUUG | 11188 | CAAAGGUCAGCUUGUCUGG |
| siRNA 4835 | 4835 | CAGACAAGCUGACCUUUGA | 11189 | UCAAAGGUCAGCUUGUCUG |
| siRNA 4836 | 4836 | AGACAAGCUGACCUUUGAG | 11190 | CUCAAAGGUCAGCUUGUCU |
| siRNA 4837 | 4837 | GACAAGCUGACCUUUGAGU | 11191 | ACUCAAAGGUCAGCUUGUC |
| siRNA 4838 | 4838 | ACAAGCUGACCUUUGAGUU | 11192 | AACUCAAAGGUCAGCUUGU |
| siRNA 4839 | 4839 | CAAGCUGACCUUUGAGUUA | 11193 | UAACUCAAAGGUCAGCUUG |
| siRNA 4840 | 4840 | AAGCUGACCUUUGAGUUA | 11194 | UUAACUCAAAGGUCAGCUU |
| | | A | | |
| siRNA 4841 | 4841 | AGCUGACCUUUGAGUUAA | 11195 | AUUAACUCAAAGGUCAGCU |
| | | U | | |
| siRNA 4842 | 4842 | GCUGACCUUUGAGUUAAU | 11196 | AAUUAACUCAAAGGUCAGC |
| | | U | | |
| siRNA 4843 | 4843 | CUGACCUUUGAGUUAAUU | 11197 | AAAUUAACUCAAAGGUCA |
| | | U | | G |
| siRNA 4844 | 4844 | UGACCUUUGAGUUAAUUU | 11198 | GAAAUUAACUCAAAGGUC |
| | | C | | A |
| siRNA 4845 | 4845 | GACCUUUGAGUUAAUUUC | 11199 | UGAAAUUAACUCAAAGGU |
| | | A | | C |
| siRNA 4846 | 4846 | ACCUUUGAGUUAAUUUCA | 11200 | CUGAAAUUAACUCAAAGG |
| | | G | | U |
| siRNA 4847 | 4847 | CCUUUGAGUUAAUUUCAGC | 11201 | GCUGAAAUUAACUCAAAG |
| | | | | G |
| siRNA 4848 | 4848 | CUUUGAGUUAAUUUCAGC | 11202 | UGCUGAAAUUAACUCAAA |
| | | A | | G |
| siRNA 4849 | 4849 | UUUGAGUUAAUUUCAGCA | 11203 | GUGCUGAAAUUAACUCAA |
| | | C | | A |
| siRNA 4850 | 4850 | UUGAGUUAAUUUCAGCAC | 11204 | UGUGCUGAAAUUAACUCA |
| | | A | | A |
| siRNA 4851 | 4851 | UGAGUUAAUUUCAGCACA | 11205 | UUGUGCUGAAAUUAACUC |
| | | A | | A |
| siRNA 4852 | 4852 | GAGUUAAUUUCAGCACAAC | 11206 | GUUGUGCUGAAAUUAACU |
| | | | | C |
| siRNA 4853 | 4853 | AGUUAAUUUCAGCACAACU | 11207 | AGUUGUGCUGAAAUUAAC |
| | | | | U |
| siRNA 4854 | 4854 | GUUAAUUUCAGCACAACUC | 11208 | GAGUUGUGCUGAAAUUAA |
| | | | | C |
| siRNA 4855 | 4855 | UUAAUUUCAGCACAACUCA | 11209 | UGAGUUGUGCUGAAAUUA |
| | | | | A |
| siRNA 4856 | 4856 | UAAUUUCAGCACAACUCAU | 11210 | AUGAGUUGUGCUGAAAUU |
| | | | | A |
| siRNA 4857 | 4857 | AAUUUCAGCACAACUCAUU | 11211 | AAUGAGUUGUGCUGAAAU |
| | | | | U |
| siRNA 4858 | 4858 | AUUUCAGCACAACUCAUUC | 11212 | GAAUGAGUUGUGCUGAAA |
| | | | | U |
| siRNA 4859 | 4859 | UUUCAGCACAACUCAUUCU | 11213 | AGAAUGAGUUGUGCUGAA |
| | | | | A |
| siRNA 4860 | 4860 | UUCAGCACAACUCAUUCUU | 11214 | AAGAAUGAGUUGUGCUGA |
| | | | | A |
| siRNA 4861 | 4861 | UCAGCACAACUCAUUCUUC | 11215 | GAAGAAUGAGUUGUGCUG |
| | | | | A |
| siRNA 4862 | 4862 | CAGCACAACUCAUUCUUCA | 11216 | UGAAGAAUGAGUUGUGCU |
| | | | | G |
| siRNA 4863 | 4863 | AGCACAACUCAUUCUUCAG | 11217 | CUGAAGAAUGAGUUGUGC |
| | | | | U |
| siRNA 4864 | 4864 | GCACAACUCAUUCUUCAGU | 11218 | ACUGAAGAAUGAGUUGUG |
| | | | | C |
| siRNA 4865 | 4865 | CACAACUCAUUCUUCAGUG | 11219 | CACUGAAGAAUGAGUUGU |
| | | | | G |
| siRNA 4866 | 4866 | ACAACUCAUUCUUCAGUGC | 11220 | GCACUGAAGAAUGAGUUG |
| | | | | U |
| siRNA 4867 | 4867 | CAACUCAUUCUUCAGUGCC | 11221 | GGCACUGAAGAAUGAGUU |
| | | | | G |
| siRNA 4868 | 4868 | AACUCAUUCUUCAGUGCCU | 11222 | AGGCACUGAAGAAUGAGU |
| | | | | U |
| siRNA 4869 | 4869 | ACUCAUUCUUCAGUGCCUC | 11223 | GAGGCACUGAAGAAUGAG |
| | | | | U |
| siRNA 4870 | 4870 | CUCAUUCUUCAGUGCCUCA | 11224 | UGAGGCACUGAAGAAUGA |
| | | | | G |
| siRNA 4871 | 4871 | UCAUUCUUCAGUGCCUCAU | 11225 | AUGAGGCACUGAAGAAUG |
| | | | | A |
| siRNA 4872 | 4872 | CAUUCUUCAGUGCCUCAUG | 11226 | CAUGAGGCACUGAAGAAU |
| | | | | G |
| siRNA 4873 | 4873 | AUUCUUCAGUGCCUCAUGA | 11227 | UCAUGAGGCACUGAAGAA |
| | | | | U |
| siRNA 4874 | 4874 | UUCUUCAGUGCCUCAUGAC | 11228 | GUCAUGAGGCACUGAAGA |
| | | | | A |
| siRNA 4875 | 4875 | UCUUCAGUGCCUCAUGACU | 11229 | AGUCAUGAGGCACUGAAG |
| | | | | A |
| siRNA 4876 | 4876 | CUUCAGUGCCUCAUGACUG | 11230 | CAGUCAUGAGGCACUGAAG |
| siRNA 4877 | 4877 | UUCAGUGCCUCAUGACUGA | 11231 | UCAGUCAUGAGGCACUGAA |
| siRNA 4878 | 4878 | UCAGUGCCUCAUGACUGAA | 11232 | UUCAGUCAUGAGGCACUGA |
| siRNA 4879 | 4879 | CAGUGCCUCAUGACUGAAA | 11233 | UUUCAGUCAUGAGGCACUG |
| siRNA 4880 | 4880 | AGUGCCUCAUGACUGAAAA | 11234 | UUUUCAGUCAUGAGGCACU |
| siRNA 4881 | 4881 | GUGCCUCAUGACUGAAAAC | 11235 | GUUUUCAGUCAUGAGGCAC |
| siRNA 4882 | 4882 | UGCCUCAUGACUGAAAACA | 11236 | UGUUUUCAGUCAUGAGGC |
| | | | | A |
| siRNA 4883 | 4883 | GCCUCAUGACUGAAAACAA | 11237 | UUGUUUUCAGUCAUGAGG |
| | | | | C |
| siRNA 4884 | 4884 | CCUCAUGACUGAAAACAAA | 11238 | UUUGUUUUCAGUCAUGAG |
| | | | | G |
| siRNA 4885 | 4885 | CUCAUGACUGAAAACAAAA | 11239 | UUUUGUUUUCAGUCAUGA |
| | | | | G |
| siRNA 4886 | 4886 | UCAUGACUGAAAACAAAA | 11240 | UUUUUGUUUUCAGUCAUG |
| | | A | | A |
| siRNA 4887 | 4887 | CAUGACUGAAAACAAAAA | 11241 | UUUUUUGUUUUCAGUCAU |
| | | A | | G |
| siRNA 4888 | 4888 | AUGACUGAAAACAAAAAA | 11242 | GUUUUUUGUUUUCAGUCA |
| | | C | | U |
| siRNA 4889 | 4889 | UGACUGAAAACAAAAAAC | 11243 | UGUUUUUUGUUUUCAGUC |
| | | A | | A |
| siRNA 4890 | 4890 | GACUGAAAACAAAAAACA | 11244 | UUGUUUUUUGUUUUCAGU |
| | | A | | C |
| siRNA 4891 | 4891 | ACUGAAAACAAAAAACAA | 11245 | UUUGUUUUUUGUUUUCAG |
| | | A | | U |
| siRNA 4892 | 4892 | CUGAAAACAAAAAACAAA | 11246 | UUUUGUUUUUUGUUUUCA |
| | | A | | G |
| siRNA 4893 | 4893 | UGAAAACAAAAAACAAAA | 11247 | UUUUUGUUUUUUGUUUUC |
| | | A | | A |
| siRNA 4894 | 4894 | GAAAACAAAAAACAAAAA | 11248 | UUUUUUGUUUUUUGUUUU |
| | | A | | C |
| siRNA 4895 | 4895 | AAAACAAAAAACAAAAAA | 11249 | UUUUUUUGUUUUUUGUUU |
| | | A | | U |
| siRNA 4896 | 4896 | AAACAAAAAACAAAAAAA | 11250 | GUUUUUUUGUUUUUUGUU |
| | | C | | U |
| siRNA 4897 | 4897 | AACAAAAAACAAAAAAAC | 11251 | CGUUUUUUUGUUUUUUGU |
| | | G | | U |
| siRNA 4898 | 4898 | ACAAAAAACAAAAAAACG | 11252 | UCGUUUUUUUGUUUUUUG |
| | | A | | U |
| siRNA 4899 | 4899 | CAAAAAACAAAAAAACGA | 11253 | UUCGUUUUUUUGUUUUUU |
| | | A | | G |
| siRNA 4900 | 4900 | AAAAAACAAAAAAACGAA | 11254 | UUUCGUUUUUUUGUUUUU |
| | | A | | U |
| siRNA 4901 | 4901 | AAAAACAAAAAAACGAAA | 11255 | CUUUCGUUUUUUUGUUUU |
| | | G | | U |
| siRNA 4902 | 4902 | AAAACAAAAAAACGAAAG | 11256 | GCUUUCGUUUUUUUGUUU |
| | | C | | U |
| siRNA 4903 | 4903 | AAACAAAAAAACGAAAGC | 11257 | UGCUUUCGUUUUUUUGUU |
| | | A | | U |
| siRNA 4904 | 4904 | AACAAAAAAACGAAAGCA | 11258 | AUGCUUUCGUUUUUUUGU |
| | | U | | U |
| siRNA 4905 | 4905 | ACAAAAAAACGAAAGCAUC | 11259 | GAUGCUUUCGUUUUUUUG |
| | | | | U |
| siRNA 4906 | 4906 | CAAAAAAACGAAAGCAUCU | 11260 | AGAUGCUUUCGUUUUUUU |
| | | | | G |
| siRNA 4907 | 4907 | AAAAAAACGAAAGCAUCU | 11261 | AAGAUGCUUUCGUUUUUU |
| | | U | | U |
| siRNA 4908 | 4908 | AAAAAACGAAAGCAUCUUC | 11262 | GAAGAUGCUUUCGUUUUU |
| | | | | U |
| siRNA 4909 | 4909 | AAAAACGAAAGCAUCUUCA | 11263 | UGAAGAUGCUUUCGUUUU |
| | | | | U |
| siRNA 4910 | 4910 | AAAACGAAAGCAUCUUCAC | 11264 | GUGAAGAUGCUUUCGUUU |
| | | | | U |
| siRNA 4911 | 4911 | AAACGAAAGCAUCUUCACA | 11265 | UGUGAAGAUGCUUUCGUU |
| | | | | U |
| siRNA 4912 | 4912 | AACGAAAGCAUCUUCACAA | 11266 | UUGUGAAGAUGCUUUCGU |
| | | | | U |
| siRNA 4913 | 4913 | ACGAAAGCAUCUUCACAAU | 11267 | AUUGUGAAGAUGCUUUCG |
| | | | | U |
| siRNA 4914 | 4914 | CGAAAGCAUCUUCACAAUG | 11268 | CAUUGUGAAGAUGCUUUC |
| | | | | G |
| siRNA 4915 | 4915 | GAAAGCAUCUUCACAAUGA | 11269 | UCAUUGUGAAGAUGCUUU |
| | | | | C |
| siRNA 4916 | 4916 | AAAGCAUCUUCACAAUGAA | 11270 | UUCAUUGUGAAGAUGCUU |
| | | | | U |
| siRNA 4917 | 4917 | AAGCAUCUUCACAAUGAAG | 11271 | CUUCAUUGUGAAGAUGCU |
| | | | | U |
| siRNA 4918 | 4918 | AGCAUCUUCACAAUGAAGC | 11272 | GCUUCAUUGUGAAGAUGC |
| | | | | U |
| siRNA 4919 | 4919 | GCAUCUUCACAAUGAAGCU | 11273 | AGCUUCAUUGUGAAGAUG |
| | | | | C |
| siRNA 4920 | 4920 | CAUCUUCACAAUGAAGCUU | 11274 | AAGCUUCAUUGUGAAGAU |
| | | | | G |
| siRNA 4921 | 4921 | AUCUUCACAAUGAAGCUUC | 11275 | GAAGCUUCAUUGUGAAGA |
| | | | | U |
| siRNA 4922 | 4922 | UCUUCACAAUGAAGCUUCC | 11276 | GGAAGCUUCAUUGUGAAG |
| | | | | A |
| siRNA 4923 | 4923 | CUUCACAAUGAAGCUUCCA | 11277 | UGGAAGCUUCAUUGUGAA |
| | | | | G |
| siRNA 4924 | 4924 | UUCACAAUGAAGCUUCCAG | 11278 | CUGGAAGCUUCAUUGUGA |
| | | | | A |
| siRNA 4925 | 4925 | UCACAAUGAAGCUUCCAGA | 11279 | UCUGGAAGCUUCAUUGUG |
| | | | | A |
| siRNA 4926 | 4926 | CACAAUGAAGCUUCCAGAU | 11280 | AUCUGGAAGCUUCAUUGU |
| | | | | G |
| siRNA 4927 | 4927 | ACAAUGAAGCUUCCAGAUA | 11281 | UAUCUGGAAGCUUCAUUG |
| | | | | U |
| siRNA 4928 | 4928 | CAAUGAAGCUUCCAGAUAG | 11282 | CUAUCUGGAAGCUUCAUUG |
| siRNA 4929 | 4929 | AAUGAAGCUUCCAGAUAGC | 11283 | GCUAUCUGGAAGCUUCAUU |
| siRNA 4930 | 4930 | AUGAAGCUUCCAGAUAGCA | 11284 | UGCUAUCUGGAAGCUUCAU |
| siRNA 4931 | 4931 | UGAAGCUUCCAGAUAGCAC | 11285 | GUGCUAUCUGGAAGCUUCA |
| siRNA 4932 | 4932 | GAAGCUUCCAGAUAGCACC | 11286 | GGUGCUAUCUGGAAGCUUC |
| siRNA 4933 | 4933 | AAGCUUCCAGAUAGCACCG | 11287 | CGGUGCUAUCUGGAAGCUU |
| siRNA 4934 | 4934 | AGCUUCCAGAUAGCACCGU | 11288 | ACGGUGCUAUCUGGAAGCU |
| siRNA 4935 | 4935 | GCUUCCAGAUAGCACCGUU | 11289 | AACGGUGCUAUCUGGAAGC |
| siRNA 4936 | 4936 | CUUCCAGAUAGCACCGUUU | 11290 | AAACGGUGCUAUCUGGAA |
| | | | | G |
| siRNA 4937 | 4937 | UUCCAGAUAGCACCGUUUU | 11291 | AAAACGGUGCUAUCUGGA |
| | | | | A |
| siRNA 4938 | 4938 | UCCAGAUAGCACCGUUUUG | 11292 | CAAAACGGUGCUAUCUGGA |
| siRNA 4939 | 4939 | CCAGAUAGCACCGUUUUGC | 11293 | GCAAAACGGUGCUAUCUGG |
| siRNA 4940 | 4940 | CAGAUAGCACCGUUUUGCU | 11294 | AGCAAAACGGUGCUAUCUG |
| siRNA 4941 | 4941 | AGAUAGCACCGUUUUGCUA | 11295 | UAGCAAAACGGUGCUAUCU |
| siRNA 4942 | 4942 | GAUAGCACCGUUUUGCUAA | 11296 | UUAGCAAAACGGUGCUAUC |
| siRNA 4943 | 4943 | AUAGCACCGUUUUGCUAAA | 11297 | UUUAGCAAAACGGUGCUA |
| | | | | U |
| siRNA 4944 | 4944 | UAGCACCGUUUUGCUAAAA | 11298 | UUUUAGCAAAACGGUGCU |
| | | | | A |
| siRNA 4945 | 4945 | AGCACCGUUUUGCUAAAAG | 11299 | CUUUUAGCAAAACGGUGCU |
| siRNA 4946 | 4946 | GCACCGUUUUGCUAAAAGA | 11300 | UCUUUUAGCAAAACGGUGC |
| siRNA 4947 | 4947 | CACCGUUUUGCUAAAAGAU | 11301 | AUCUUUUAGCAAAACGGU |
| | | | | G |
| siRNA 4948 | 4948 | ACCGUUUUGCUAAAAGAU | 11302 | UAUCUUUUAGCAAAACGG |
| | | A | | U |
| siRNA 4949 | 4949 | CCGUUUUGCUAAAAGAUAC | 11303 | GUAUCUUUUAGCAAAACG |
| | | | | G |
| siRNA 4950 | 4950 | CGUUUUGCUAAAAGAUAC | 11304 | UGUAUCUUUUAGCAAAAC |
| | | A | | G |
| siRNA 4951 | 4951 | GUUUUGCUAAAAGAUACA | 11305 | AUGUAUCUUUUAGCAAAA |
| | | U | | C |
| siRNA 4952 | 4952 | UUUUGCUAAAAGAUACAU | 11306 | AAUGUAUCUUUUAGCAAA |
| | | U | | A |
| siRNA 4953 | 4953 | UUUGCUAAAAGAUACAUU | 11307 | GAAUGUAUCUUUUAGCAA |
| | | C | | A |
| siRNA 4954 | 4954 | UUGCUAAAAGAUACAUUC | 11308 | AGAAUGUAUCUUUUAGCA |
| | | U | | A |
| siRNA 4955 | 4955 | UGCUAAAAGAUACAUUCUC | 11309 | GAGAAUGUAUCUUUUAGC |
| | | | | A |
| siRNA 4956 | 4956 | GCUAAAAGAUACAUUCUCA | 11310 | UGAGAAUGUAUCUUUUAG |
| | | | | C |
| siRNA 4957 | 4957 | CUAAAAGAUACAUUCUCAU | 11311 | AUGAGAAUGUAUCUUUUA |
| | | | | G |
| siRNA 4958 | 4958 | UAAAAGAUACAUUCUCAU | 11312 | AAUGAGAAUGUAUCUUUU |
| | | U | | A |
| siRNA 4959 | 4959 | AAAAGAUACAUUCUCAUU | 11313 | CAAUGAGAAUGUAUCUUU |
| | | G | | U |
| siRNA 4960 | 4960 | AAAGAUACAUUCUCAUUG | 11314 | ACAAUGAGAAUGUAUCUU |
| | | U | | U |
| siRNA 4961 | 4961 | AAGAUACAUUCUCAUUGU | 11315 | AACAAUGAGAAUGUAUCU |
| | | U | | U |
| siRNA 4962 | 4962 | AGAUACAUUCUCAUUGUU | 11316 | AAACAAUGAGAAUGUAUC |
| | | U | | U |
| siRNA 4963 | 4963 | GAUACAUUCUCAUUGUUU | 11317 | AAAACAAUGAGAAUGUAU |
| | | U | | C |
| siRNA 4964 | 4964 | AUACAUUCUCAUUGUUUUC | 11318 | GAAAACAAUGAGAAUGUA |
| | | | | U |
| siRNA 4965 | 4965 | UACAUUCUCAUUGUUUUCC | 11319 | GGAAAACAAUGAGAAUGU |
| | | | | A |
| siRNA 4966 | 4966 | ACAUUCUCAUUGUUUUCCA | 11320 | UGGAAAACAAUGAGAAUG |
| | | | | U |
| siRNA 4967 | 4967 | CAUUCUCAUUGUUUUCCAA | 11321 | UUGGAAAACAAUGAGAAU |
| | | | | G |
| siRNA 4968 | 4968 | AUUCUCAUUGUUUUCCAAC | 11322 | GUUGGAAAACAAUGAGAA |
| | | | | U |
| siRNA 4969 | 4969 | UUCUCAUUGUUUUCCAACA | 11323 | UGUUGGAAAACAAUGAGA |
| | | | | A |
| siRNA 4970 | 4970 | UCUCAUUGUUUUCCAACAG | 11324 | CUGUUGGAAAACAAUGAG |
| | | | | A |
| siRNA 4971 | 4971 | CUCAUUGUUUUCCAACAGU | 11325 | ACUGUUGGAAAACAAUGA |
| | | | | G |
| siRNA 4972 | 4972 | UCAUUGUUUUCCAACAGUG | 11326 | CACUGUUGGAAAACAAUG |
| | | | | A |
| siRNA 4973 | 4973 | CAUUGUUUUCCAACAGUGA | 11327 | UCACUGUUGGAAAACAAU |
| | | | | G |
| siRNA 4974 | 4974 | AUUGUUUUCCAACAGUGA | 11328 | AUCACUGUUGGAAAACAA |
| | | U | | U |
| siRNA 4975 | 4975 | UUGUUUUCCAACAGUGAU | 11329 | CAUCACUGUUGGAAAACAA |
| | | G | | |
| siRNA 4976 | 4976 | UGUUUUCCAACAGUGAUG | 11330 | CCAUCACUGUUGGAAAACA |
| | | G | | |
| siRNA 4977 | 4977 | GUUUUCCAACAGUGAUGGC | 11331 | GCCAUCACUGUUGGAAAAC |
| siRNA 4978 | 4978 | UUUUCCAACAGUGAUGGCU | 11332 | AGCCAUCACUGUUGGAAAA |
| siRNA 4979 | 4979 | UUUCCAACAGUGAUGGCUU | 11333 | AAGCCAUCACUGUUGGAAA |
| siRNA 4980 | 4980 | UUCCAACAGUGAUGGCUUC | 11334 | GAAGCCAUCACUGUUGGAA |
| siRNA 4981 | 4981 | UCCAACAGUGAUGGCUUCC | 11335 | GGAAGCCAUCACUGUUGGA |
| siRNA 4982 | 4982 | CCAACAGUGAUGGCUUCCA | 11336 | UGGAAGCCAUCACUGUUGG |
| siRNA 4983 | 4983 | CAACAGUGAUGGCUUCCAC | 11337 | GUGGAAGCCAUCACUGUUG |
| siRNA 4984 | 4984 | AACAGUGAUGGCUUCCACA | 11338 | UGUGGAAGCCAUCACUGUU |
| siRNA 4985 | 4985 | ACAGUGAUGGCUUCCACAU | 11339 | AUGUGGAAGCCAUCACUGU |
| siRNA 4986 | 4986 | CAGUGAUGGCUUCCACAUA | 11340 | UAUGUGGAAGCCAUCACUG |
| siRNA 4987 | 4987 | AGUGAUGGCUUCCACAUAA | 11341 | UUAUGUGGAAGCCAUCACU |
| siRNA 4988 | 4988 | GUGAUGGCUUCCACAUAAG | 11342 | CUUAUGUGGAAGCCAUCAC |
| siRNA 4989 | 4989 | UGAUGGCUUCCACAUAAGG | 11343 | CCUUAUGUGGAAGCCAUCA |
| siRNA 4990 | 4990 | GAUGGCUUCCACAUAAGGU | 11344 | ACCUUAUGUGGAAGCCAUC |
| siRNA 4991 | 4991 | AUGGCUUCCACAUAAGGUU | 11345 | AACCUUAUGUGGAAGCCAU |
| siRNA 4992 | 4992 | UGGCUUCCACAUAAGGUUA | 11346 | UAACCUUAUGUGGAAGCCA |
| siRNA 4993 | 4993 | GGCUUCCACAUAAGGUUAA | 11347 | UUAACCUUAUGUGGAAGCC |
| siRNA 4994 | 4994 | GCUUCCACAUAAGGUUAAA | 11348 | UUUAACCUUAUGUGGAAG |
| | | | | C |
| siRNA 4995 | 4995 | CUUCCACAUAAGGUUAAAC | 11349 | GUUUAACCUUAUGUGGAA |
| | | | | G |
| siRNA 4996 | 4996 | UUCCACAUAAGGUUAAACA | 11350 | UGUUUAACCUUAUGUGGA |
| | | | | A |
| siRNA 4997 | 4997 | UCCACAUAAGGUUAAACAA | 11351 | UUGUUUAACCUUAUGUGG |
| | | | | A |
| siRNA 4998 | 4998 | CCACAUAAGGUUAAACAAA | 11352 | UUUGUUUAACCUUAUGUG |
| | | | | G |
| siRNA 4999 | 4999 | CACAUAAGGUUAAACAAAC | 11353 | GUUUGUUUAACCUUAUGU |
| | | | | G |
| siRNA 5000 | 5000 | ACAUAAGGUUAAACAAAC | 11354 | AGUUUGUUUAACCUUAUG |
| | | U | | U |
| siRNA 5001 | 5001 | CAUAAGGUUAAACAAACU | 11355 | UAGUUUGUUUAACCUUAU |
| | | A | | G |
| siRNA 5002 | 5002 | AUAAGGUUAAACAAACUA | 11356 | CUAGUUUGUUUAACCUUA |
| | | G | | U |
| siRNA 5003 | 5003 | UAAGGUUAAACAAACUAG | 11357 | CCUAGUUUGUUUAACCUUA |
| | | G | | |
| siRNA 5004 | 5004 | AAGGUUAAACAAACUAGG | 11358 | ACCUAGUUUGUUUAACCUU |
| | | U | | |
| siRNA 5005 | 5005 | AGGUUAAACAAACUAGGU | 11359 | CACCUAGUUUGUUUAACCU |
| | | G | | |
| siRNA 5006 | 5006 | GGUUAAACAAACUAGGUG | 11360 | GCACCUAGUUUGUUUAACC |
| | | C | | |
| siRNA 5007 | 5007 | GUUAAACAAACUAGGUGC | 11361 | AGCACCUAGUUUGUUUAAC |
| | | U | | |
| siRNA 5008 | 5008 | UUAAACAAACUAGGUGCU | 11362 | AAGCACCUAGUUUGUUUA |
| | | U | | A |
| siRNA 5009 | 5009 | UAAACAAACUAGGUGCUU | 11363 | CAAGCACCUAGUUUGUUUA |
| | | G | | |
| siRNA 5010 | 5010 | AAACAAACUAGGUGCUUG | 11364 | ACAAGCACCUAGUUUGUUU |
| | | U | | |
| siRNA 5011 | 5011 | AACAAACUAGGUGCUUGU | 11365 | UACAAGCACCUAGUUUGUU |
| | | A | | |
| siRNA 5012 | 5012 | ACAAACUAGGUGCUUGUA | 11366 | UUACAAGCACCUAGUUUGU |
| | | A | | |
| siRNA 5013 | 5013 | CAAACUAGGUGCUUGUAA | 11367 | UUUACAAGCACCUAGUUUG |
| | | A | | |
| siRNA 5014 | 5014 | AAACUAGGUGCUUGUAAA | 11368 | AUUUACAAGCACCUAGUUU |
| | | U | | |
| siRNA 5015 | 5015 | AACUAGGUGCUUGUAAAU | 11369 | UAUUUACAAGCACCUAGUU |
| | | A | | |
| siRNA 5016 | 5016 | ACUAGGUGCUUGUAAAUA | 11370 | UUAUUUACAAGCACCUAGU |
| | | A | | |
| siRNA 5017 | 5017 | CUAGGUGCUUGUAAAUAA | 11371 | AUUAUUUACAAGCACCUAG |
| | | U | | |
| siRNA 5018 | 5018 | UAGGUGCUUGUAAAUAAU | 11372 | AAUUAUUUACAAGCACCUA |
| | | U | | |
| siRNA 5019 | 5019 | AGGUGCUUGUAAAUAAUU | 11373 | AAAUUAUUUACAAGCACCU |
| | | U | | |
| siRNA 5020 | 5020 | GGUGCUUGUAAAUAAUUU | 11374 | UAAAUUAUUUACAAGCACC |
| | | A | | |
| siRNA 5021 | 5021 | GUGCUUGUAAAUAAUUUA | 11375 | AUAAAUUAUUUACAAGCA |
| | | U | | C |
| siRNA 5022 | 5022 | UGCUUGUAAAUAAUUUAU | 11376 | AAUAAAUUAUUUACAAGC |
| | | U | | A |
| siRNA 5023 | 5023 | GCUUGUAAAUAAUUUAUU | 11377 | UAAUAAAUUAUUUACAAG |
| | | A | | C |
| siRNA 5024 | 5024 | CUUGUAAAUAAUUUAUUA | 11378 | GUAAUAAAUUAUUUACAA |
| | | C | | G |
| siRNA 5025 | 5025 | UUGUAAAUAAUUUAUUAC | 11379 | UGUAAUAAAUUAUUUACA |
| | | A | | A |
| siRNA 5026 | 5026 | UGUAAAUAAUUUAUUACA | 11380 | CUGUAAUAAAUUAUUUAC |
| | | G | | A |
| siRNA 5027 | 5027 | GUAAAUAAUUUAUUACAG | 11381 | ACUGUAAUAAAUUAUUUA |
| | | U | | C |
| siRNA 5028 | 5028 | UAAAUAAUUUAUUACAGU | 11382 | AACUGUAAUAAAUUAUUU |
| | | U | | A |
| siRNA 5029 | 5029 | AAAUAAUUUAUUACAGUU | 11383 | AAACUGUAAUAAAUUAUU |
| | | U | | U |
| siRNA 5030 | 5030 | AAUAAUUUAUUACAGUUU | 11384 | UAAACUGUAAUAAAUUAU |
| | | A | | U |
| siRNA 5031 | 5031 | AUAAUUUAUUACAGUUUA | 11385 | GUAAACUGUAAUAAAUUA |
| | | C | | U |
| siRNA 5032 | 5032 | UAAUUUAUUACAGUUUAC | 11386 | AGUAAACUGUAAUAAAUU |
| | | U | | A |
| siRNA 5033 | 5033 | AAUUUAUUACAGUUUACU | 11387 | GAGUAAACUGUAAUAAAU |
| | | C | | U |
| siRNA 5034 | 5034 | AUUUAUUACAGUUUACUC | 11388 | AGAGUAAACUGUAAUAAA |
| | | U | | U |
| siRNA 5035 | 5035 | UUUAUUACAGUUUACUCU | 11389 | UAGAGUAAACUGUAAUAA |
| | | A | | A |
| siRNA 5036 | 5036 | UUAUUACAGUUUACUCUA | 11390 | AUAGAGUAAACUGUAAUA |
| | | U | | A |
| siRNA 5037 | 5037 | UAUUACAGUUUACUCUAUC | 11391 | GAUAGAGUAAACUGUAAU |
| | | | | A |
| siRNA 5038 | 5038 | AUUACAGUUUACUCUAUCG | 11392 | CGAUAGAGUAAACUGUAA |
| | | | | U |
| siRNA 5039 | 5039 | UUACAGUUUACUCUAUCGC | 11393 | GCGAUAGAGUAAACUGUA |
| | | | | A |
| siRNA 5040 | 5040 | UACAGUUUACUCUAUCGCA | 11394 | UGCGAUAGAGUAAACUGU |
| | | | | A |
| siRNA 5041 | 5041 | ACAGUUUACUCUAUCGCAU | 11395 | AUGCGAUAGAGUAAACUG |
| | | | | U |
| siRNA 5042 | 5042 | CAGUUUACUCUAUCGCAUU | 11396 | AAUGCGAUAGAGUAAACU |
| | | | | G |
| siRNA 5043 | 5043 | AGUUUACUCUAUCGCAUUU | 11397 | AAAUGCGAUAGAGUAAAC |
| | | | | U |
| siRNA 5044 | 5044 | GUUUACUCUAUCGCAUUUC | 11398 | GAAAUGCGAUAGAGUAAA |
| | | | | C |
| siRNA 5045 | 5045 | UUUACUCUAUCGCAUUUCU | 11399 | AGAAAUGCGAUAGAGUAA |
| | | | | A |
| siRNA 5046 | 5046 | UUACUCUAUCGCAUUUCUG | 11400 | CAGAAAUGCGAUAGAGUA |
| | | | | A |
| siRNA 5047 | 5047 | UACUCUAUCGCAUUUCUGU | 11401 | ACAGAAAUGCGAUAGAGU |
| | | | | A |
| siRNA 5048 | 5048 | ACUCUAUCGCAUUUCUGUA | 11402 | UACAGAAAUGCGAUAGAG |
| | | | | U |
| siRNA 5049 | 5049 | CUCUAUCGCAUUUCUGUAA | 11403 | UUACAGAAAUGCGAUAGA |
| | | | | G |
| siRNA 5050 | 5050 | UCUAUCGCAUUUCUGUAAC | 11404 | GUUACAGAAAUGCGAUAG |
| | | | | A |
| siRNA 5051 | 5051 | CUAUCGCAUUUCUGUAACA | 11405 | UGUUACAGAAAUGCGAUA |
| | | | | G |
| siRNA 5052 | 5052 | UAUCGCAUUUCUGUAACAU | 11406 | AUGUUACAGAAAUGCGAU |
| | | | | A |
| siRNA 5053 | 5053 | AUCGCAUUUCUGUAACAUG | 11407 | CAUGUUACAGAAAUGCGA |
| | | | | U |
| siRNA 5054 | 5054 | UCGCAUUUCUGUAACAUGA | 11408 | UCAUGUUACAGAAAUGCG |
| | | | | A |
| siRNA 5055 | 5055 | CGCAUUUCUGUAACAUGAA | 11409 | UUCAUGUUACAGAAAUGC |
| | | | | G |
| siRNA 5056 | 5056 | GCAUUUCUGUAACAUGAA | 11410 | UUUCAUGUUACAGAAAUG |
| | | A | | C |
| siRNA 5057 | 5057 | CAUUUCUGUAACAUGAAA | 11411 | AUUUCAUGUUACAGAAAU |
| | | U | | G |
| siRNA 5058 | 5058 | AUUUCUGUAACAUGAAAU | 11412 | CAUUUCAUGUUACAGAAA |
| | | G | | U |
| siRNA 5059 | 5059 | UUUCUGUAACAUGAAAUG | 11413 | GCAUUUCAUGUUACAGAA |
| | | C | | A |
| siRNA 5060 | 5060 | UUCUGUAACAUGAAAUGC | 11414 | UGCAUUUCAUGUUACAGA |
| | | A | | A |
| siRNA 5061 | 5061 | UCUGUAACAUGAAAUGCA | 11415 | AUGCAUUUCAUGUUACAG |
| | | U | | A |
| siRNA 5062 | 5062 | CUGUAACAUGAAAUGCAU | 11416 | CAUGCAUUUCAUGUUACAG |
| | | G | | |
| siRNA 5063 | 5063 | UGUAACAUGAAAUGCAUG | 11417 | GCAUGCAUUUCAUGUUACA |
| | | C | | |
| siRNA 5064 | 5064 | GUAACAUGAAAUGCAUGCC | 11418 | GGCAUGCAUUUCAUGUUAC |
| siRNA 5065 | 5065 | UAACAUGAAAUGCAUGCCC | 11419 | GGGCAUGCAUUUCAUGUU |
| | | | | A |
| siRNA 5066 | 5066 | AACAUGAAAUGCAUGCCCU | 11420 | AGGGCAUGCAUUUCAUGU |
| | | | | U |
| siRNA 5067 | 5067 | ACAUGAAAUGCAUGCCCUU | 11421 | AAGGGCAUGCAUUUCAUG |
| | | | | U |
| siRNA 5068 | 5068 | CAUGAAAUGCAUGCCCUUC | 11422 | GAAGGGCAUGCAUUUCAU |
| | | | | G |
| siRNA 5069 | 5069 | AUGAAAUGCAUGCCCUUCU | 11423 | AGAAGGGCAUGCAUUUCA |
| | | | | U |
| siRNA 5070 | 5070 | UGAAAUGCAUGCCCUUCUU | 11424 | AAGAAGGGCAUGCAUUUC |
| | | | | A |
| siRNA 5071 | 5071 | GAAAUGCAUGCCCUUCUUC | 11425 | GAAGAAGGGCAUGCAUUU |
| | | | | C |
| siRNA 5072 | 5072 | AAAUGCAUGCCCUUCUUCA | 11426 | UGAAGAAGGGCAUGCAUU |
| | | | | U |
| siRNA 5073 | 5073 | AAUGCAUGCCCUUCUUCAG | 11427 | CUGAAGAAGGGCAUGCAU |
| | | | | U |
| siRNA 5074 | 5074 | AUGCAUGCCCUUCUUCAGG | 11428 | CCUGAAGAAGGGCAUGCAU |
| siRNA 5075 | 5075 | UGCAUGCCCUUCUUCAGGG | 11429 | CCCUGAAGAAGGGCAUGCA |
| siRNA 5076 | 5076 | GCAUGCCCUUCUUCAGGGG | 11430 | CCCCUGAAGAAGGGCAUGC |
| siRNA 5077 | 5077 | CAUGCCCUUCUUCAGGGGA | 11431 | UCCCCUGAAGAAGGGCAUG |
| siRNA 5078 | 5078 | AUGCCCUUCUUCAGGGGAA | 11432 | UUCCCCUGAAGAAGGGCAU |
| siRNA 5079 | 5079 | UGCCCUUCUUCAGGGGAAG | 11433 | CUUCCCCUGAAGAAGGGCA |
| siRNA 5080 | 5080 | GCCCUUCUUCAGGGGAAGA | 11434 | UCUUCCCCUGAAGAAGGGC |
| siRNA 5081 | 5081 | CCCUUCUUCAGGGGAAGAC | 11435 | GUCUUCCCCUGAAGAAGGG |
| siRNA 5082 | 5082 | CCUUCUUCAGGGGAAGACU | 11436 | AGUCUUCCCCUGAAGAAGG |
| siRNA 5083 | 5083 | CUUCUUCAGGGGAAGACUG | 11437 | CAGUCUUCCCCUGAAGAAG |
| siRNA 5084 | 5084 | UUCUUCAGGGGAAGACUG | 11438 | ACAGUCUUCCCCUGAAGAA |
| | | U | | |
| siRNA 5085 | 5085 | UCUUCAGGGGAAGACUGU | 11439 | CACAGUCUUCCCCUGAAGA |
| | | G | | |
| siRNA 5086 | 5086 | CUUCAGGGGAAGACUGUG | 11440 | CCACAGUCUUCCCCUGAAG |
| | | G | | |
| siRNA 5087 | 5087 | UUCAGGGGAAGACUGUGG | 11441 | ACCACAGUCUUCCCCUGAA |
| | | U | | |
| siRNA 5088 | 5088 | UCAGGGGAAGACUGUGGU | 11442 | GACCACAGUCUUCCCCUGA |
| | | C | | |
| siRNA 5089 | 5089 | CAGGGGAAGACUGUGGUC | 11443 | UGACCACAGUCUUCCCCUG |
| | | A | | |
| siRNA 5090 | 5090 | AGGGGAAGACUGUGGUCA | 11444 | UUGACCACAGUCUUCCCCU |
| | | A | | |
| siRNA 5091 | 5091 | GGGGAAGACUGUGGUCAA | 11445 | CUUGACCACAGUCUUCCCC |
| | | G | | |
| siRNA 5092 | 5092 | GGGAAGACUGUGGUCAAG | 11446 | ACUUGACCACAGUCUUCCC |
| | | U | | |
| siRNA 5093 | 5093 | GGAAGACUGUGGUCAAGU | 11447 | AACUUGACCACAGUCUUCC |
| | | U | | |
| siRNA 5094 | 5094 | GAAGACUGUGGUCAAGUU | 11448 | UAACUUGACCACAGUCUUC |
| | | A | | |
| siRNA 5095 | 5095 | AAGACUGUGGUCAAGUUA | 11449 | UUAACUUGACCACAGUCUU |
| | | A | | |
| siRNA 5096 | 5096 | AGACUGUGGUCAAGUUAA | 11450 | UUUAACUUGACCACAGUCU |
| | | A | | |
| siRNA 5097 | 5097 | GACUGUGGUCAAGUUAAA | 11451 | UUUUAACUUGACCACAGUC |
| | | A | | |
| siRNA 5098 | 5098 | ACUGUGGUCAAGUUAAAA | 11452 | UUUUUAACUUGACCACAGU |
| | | A | | |
| siRNA 5099 | 5099 | CUGUGGUCAAGUUAAAAA | 11453 | UUUUUUAACUUGACCACAG |
| | | A | | |
| siRNA 5100 | 5100 | UGUGGUCAAGUUAAAAAA | 11454 | UUUUUUUAACUUGACCACA |
| | | A | | |
| siRNA 5101 | 5101 | GUGGUCAAGUUAAAAAAA | 11455 | UUUUUUUUAACUUGACCAC |
| | | A | | |
| siRNA 5102 | 5102 | UGGUCAAGUUAAAAAAAA | 11456 | UUUUUUUUUAACUUGACC |
| | | A | | A |
| siRNA 5103 | 5103 | GGUCAAGUUAAAAAAAAA | 11457 | UUUUUUUUUUAACUUGAC |
| | | A | | C |
| siRNA 5104 | 5104 | GUCAAGUUAAAAAAAAAA | 11458 | UUUUUUUUUUUAACUUGA |
| | | A | | C |
| siRNA 5105 | 5105 | UCAAGUUAAAAAAAAAAA | 11459 | UUUUUUUUUUUUAACUUG |
| | | A | | A |
| siRNA 5106 | 5106 | CAAGUUAAAAAAAAAAAA | 11460 | GUUUUUUUUUUUUAACUU |
| | | C | | G |
| siRNA 5107 | 5107 | AAGUUAAAAAAAAAAAAC | 11461 | UGUUUUUUUUUUUUAACU |
| | | A | | U |
| siRNA 5108 | 5108 | AGUUAAAAAAAAAAAACA | 11462 | UUGUUUUUUUUUUUUAAC |
| | | A | | U |
| siRNA 5109 | 5109 | GUUAAAAAAAAAAAACAA | 11463 | AUUGUUUUUUUUUUUUAA |
| | | | | C |
| siRNA 5110 | 5110 | UUAAAAAAAAAAAACAAU | 11464 | UAUUGUUUUUUUUUUUUA |
| | | A | | A |
| siRNA 5111 | 5111 | UAAAAAAAAAAAACAAUA | 11465 | AUAUUGUUUUUUUUUUUU |
| | | U | | A |
| siRNA 5112 | 5112 | AAAAAAAAAAAACAAUAU | 11466 | AAUAUUGUUUUUUUUUUU |
| | | U | | U |
| siRNA 5113 | 5113 | AAAAAAAAAAACAAUAUU | 11467 | UAAUAUUGUUUUUUUUUU |
| | | A | | U |
| siRNA 5114 | 5114 | AAAAAAAAAACAAUAUUA | 11468 | UUAAUAUUGUUUUUUUUU |
| | | A | | U |
| siRNA 5115 | 5115 | AAAAAAAAACAAUAUUAA | 11469 | UUUAAUAUUGUUUUUUUU |
| | | A | | U |
| siRNA 5116 | 5116 | AAAAAAAACAAUAUUAAA | 11470 | GUUUAAUAUUGUUUUUUU |
| | | C | | U |
| siRNA 5117 | 5117 | AAAAAAACAAUAUUAAAC | 11471 | UGUUUAAUAUUGUUUUUU |
| | | A | | U |
| siRNA 5118 | 5118 | AAAAAACAAUAUUAAACA | 11472 | UUGUUUAAUAUUGUUUUU |
| | | A | | U |
| siRNA 5119 | 5119 | AAAAACAAUAUUAAACAA | 11473 | GUUGUUUAAUAUUGUUUU |
| | | C | | U |
| siRNA 5120 | 5120 | AAAACAAUAUUAAACAAC | 11474 | UGUUGUUUAAUAUUGUUU |
| | | A | | U |
| siRNA 5121 | 5121 | AAACAAUAUUAAACAACA | 11475 | AUGUUGUUUAAUAUUGUU |
| | | U | | U |
| siRNA 5122 | 5122 | AACAAUAUUAAACAACAU | 11476 | CAUGUUGUUUAAUAUUGU |
| | | G | | U |
| siRNA 5123 | 5123 | ACAAUAUUAAACAACAUG | 11477 | UCAUGUUGUUUAAUAUUG |
| | | A | | U |
| siRNA 5124 | 5124 | CAAUAUUAAACAACAUGA | 11478 | UUCAUGUUGUUUAAUAUU |
| | | A | | G |
| siRNA 5125 | 5125 | AAUAUUAAACAACAUGAA | 11479 | UUUCAUGUUGUUUAAUAU |
| | | A | | U |
| siRNA 5126 | 5126 | AUAUUAAACAACAUGAAA | 11480 | GUUUCAUGUUGUUUAAUA |
| | | C | | U |
| siRNA 5127 | 5127 | UAUUAAACAACAUGAAAC | 11481 | AGUUUCAUGUUGUUUAAU |
| | | U | | A |
| siRNA 5128 | 5128 | AUUAAACAACAUGAAACU | 11482 | CAGUUUCAUGUUGUUUAA |
| | | G | | U |
| siRNA 5129 | 5129 | UUAAACAACAUGAAACUGC | 11483 | GCAGUUUCAUGUUGUUUA |
| | | | | A |
| siRNA 5130 | 5130 | UAAACAACAUGAAACUGCA | 11484 | UGCAGUUUCAUGUUGUUU |
| | | | | A |
| siRNA 5131 | 5131 | AAACAACAUGAAACUGCAG | 11485 | CUGCAGUUUCAUGUUGUU |
| | | | | U |
| siRNA 5132 | 5132 | AACAACAUGAAACUGCAGU | 11486 | ACUGCAGUUUCAUGUUGU |
| | | | | U |
| siRNA 5133 | 5133 | ACAACAUGAAACUGCAGUC | 11487 | GACUGCAGUUUCAUGUUG |
| | | | | U |
| siRNA 5134 | 5134 | CAACAUGAAACUGCAGUCU | 11488 | AGACUGCAGUUUCAUGUU |
| | | | | G |
| siRNA 5135 | 5135 | AACAUGAAACUGCAGUCUG | 11489 | CAGACUGCAGUUUCAUGUU |
| siRNA 5136 | 5136 | ACAUGAAACUGCAGUCUGU | 11490 | ACAGACUGCAGUUUCAUGU |
| siRNA 5137 | 5137 | CAUGAAACUGCAGUCUGUU | 11491 | AACAGACUGCAGUUUCAUG |
| siRNA 5138 | 5138 | AUGAAACUGCAGUCUGUU | 11492 | AAACAGACUGCAGUUUCAU |
| | | U | | |
| siRNA 5139 | 5139 | UGAAACUGCAGUCUGUUU | 11493 | AAAACAGACUGCAGUUUCA |
| | | U | | |
| siRNA 5140 | 5140 | GAAACUGCAGUCUGUUUU | 11494 | AAAAACAGACUGCAGUUUC |
| | | U | | |
| siRNA 5141 | 5141 | AAACUGCAGUCUGUUUUU | 11495 | CAAAAACAGACUGCAGUUU |
| | | G | | |
| siRNA 5142 | 5142 | AACUGCAGUCUGUUUUUG | 11496 | UCAAAAACAGACUGCAGUU |
| | | A | | |
| siRNA 5143 | 5143 | ACUGCAGUCUGUUUUUGA | 11497 | UUCAAAAACAGACUGCAGU |
| | | A | | |
| siRNA 5144 | 5144 | CUGCAGUCUGUUUUUGAA | 11498 | UUUCAAAAACAGACUGCAG |
| | | A | | |
| siRNA 5145 | 5145 | UGCAGUCUGUUUUUGAAA | 11499 | UUUUCAAAAACAGACUGCA |
| | | A | | |
| siRNA 5146 | 5146 | GCAGUCUGUUUUUGAAAA | 11500 | AUUUUCAAAAACAGACUGC |
| | | U | | |
| siRNA 5147 | 5147 | CAGUCUGUUUUUGAAAAU | 11501 | CAUUUUCAAAAACAGACUG |
| | | G | | |
| siRNA 5148 | 5148 | AGUCUGUUUUUGAAAAUG | 11502 | UCAUUUUCAAAAACAGACU |
| | | A | | |
| siRNA 5149 | 5149 | GUCUGUUUUUGAAAAUGA | 11503 | CUCAUUUUCAAAAACAGAC |
| | | G | | |
| siRNA 5150 | 5150 | UCUGUUUUUGAAAAUGAG | 11504 | UCUCAUUUUCAAAAACAGA |
| | | A | | |
| siRNA 5151 | 5151 | CUGUUUUUGAAAAUGAGA | 11505 | UUCUCAUUUUCAAAAACAG |
| | | A | | |
| siRNA 5152 | 5152 | UGUUUUUGAAAAUGAGAA | 11506 | AUUCUCAUUUUCAAAAACA |
| | | U | | |
| siRNA 5153 | 5153 | GUUUUUGAAAAUGAGAAU | 11507 | CAUUCUCAUUUUCAAAAAC |
| | | G | | |
| siRNA 5154 | 5154 | UUUUUGAAAAUGAGAAUG | 11508 | ACAUUCUCAUUUUCAAAAA |
| | | U | | |
| siRNA 5155 | 5155 | UUUUGAAAAUGAGAAUGU | 11509 | GACAUUCUCAUUUUCAAAA |
| | | C | | |
| siRNA 5156 | 5156 | UUUGAAAAUGAGAAUGUC | 11510 | GGACAUUCUCAUUUUCAAA |
| | | C | | |
| siRNA 5157 | 5157 | UUGAAAAUGAGAAUGUCC | 11511 | AGGACAUUCUCAUUUUCAA |
| | | U | | |
| siRNA 5158 | 5158 | UGAAAAUGAGAAUGUCCU | 11512 | UAGGACAUUCUCAUUUUCA |
| | | A | | |
| siRNA 5159 | 5159 | GAAAAUGAGAAUGUCCUA | 11513 | UUAGGACAUUCUCAUUUUC |
| | | A | | |
| siRNA 5160 | 5160 | AAAAUGAGAAUGUCCUAA | 11514 | CUUAGGACAUUCUCAUUUU |
| | | G | | |
| siRNA 5161 | 5161 | AAAUGAGAAUGUCCUAAG | 11515 | ACUUAGGACAUUCUCAUUU |
| | | U | | |
| siRNA 5162 | 5162 | AAUGAGAAUGUCCUAAGU | 11516 | CACUUAGGACAUUCUCAUU |
| | | G | | |
| siRNA 5163 | 5163 | AUGAGAAUGUCCUAAGUG | 11517 | UCACUUAGGACAUUCUCAU |
| | | A | | |
| siRNA 5164 | 5164 | UGAGAAUGUCCUAAGUGA | 11518 | AUCACUUAGGACAUUCUCA |
| | | U | | |
| siRNA 5165 | 5165 | GAGAAUGUCCUAAGUGAU | 11519 | AAUCACUUAGGACAUUCUC |
| | | U | | |
| siRNA 5166 | 5166 | AGAAUGUCCUAAGUGAUU | 11520 | GAAUCACUUAGGACAUUCU |
| | | C | | |
| siRNA 5167 | 5167 | GAAUGUCCUAAGUGAUUC | 11521 | UGAAUCACUUAGGACAUUC |
| | | A | | |
| siRNA 5168 | 5168 | AAUGUCCUAAGUGAUUCA | 11522 | CUGAAUCACUUAGGACAUU |
| | | G | | |
| siRNA 5169 | 5169 | AUGUCCUAAGUGAUUCAG | 11523 | UCUGAAUCACUUAGGACAU |
| | | A | | |
| siRNA 5170 | 5170 | UGUCCUAAGUGAUUCAGA | 11524 | UUCUGAAUCACUUAGGACA |
| | | A | | |
| siRNA 5171 | 5171 | GUCCUAAGUGAUUCAGAA | 11525 | CUUCUGAAUCACUUAGGAC |
| | | G | | |
| siRNA 5172 | 5172 | UCCUAAGUGAUUCAGAAG | 11526 | UCUUCUGAAUCACUUAGGA |
| | | A | | |
| siRNA 5173 | 5173 | CCUAAGUGAUUCAGAAGA | 11527 | CUCUUCUGAAUCACUUAGG |
| | | G | | |
| siRNA 5174 | 5174 | CUAAGUGAUUCAGAAGAG | 11528 | UCUCUUCUGAAUCACUUAG |
| | | A | | |
| siRNA 5175 | 5175 | UAAGUGAUUCAGAAGAGA | 11529 | CUCUCUUCUGAAUCACUUA |
| | | G | | |
| siRNA 5176 | 5176 | AAGUGAUUCAGAAGAGAG | 11530 | CCUCUCUUCUGAAUCACUU |
| | | G | | |
| siRNA 5177 | 5177 | AGUGAUUCAGAAGAGAGG | 11531 | UCCUCUCUUCUGAAUCACU |
| | | A | | |
| siRNA 5178 | 5178 | GUGAUUCAGAAGAGAGGA | 11532 | CUCCUCUCUUCUGAAUCAC |
| | | G | | |
| siRNA 5179 | 5179 | UGAUUCAGAAGAGAGGAG | 11533 | CCUCCUCUCUUCUGAAUCA |
| | | G | | |
| siRNA 5180 | 5180 | GAUUCAGAAGAGAGGAGG | 11534 | CCCUCCUCUCUUCUGAAUC |
| | | G | | |
| siRNA 5181 | 5181 | AUUCAGAAGAGAGGAGGG | 11535 | UCCCUCCUCUCUUCUGAAU |
| | | A | | |
| siRNA 5182 | 5182 | UUCAGAAGAGAGGAGGGA | 11536 | UUCCCUCCUCUCUUCUGAA |
| | | A | | |
| siRNA 5183 | 5183 | UCAGAAGAGAGGAGGGAA | 11537 | CUUCCCUCCUCUCUUCUGA |
| | | G | | |
| siRNA 5184 | 5184 | CAGAAGAGAGGAGGGAAG | 11538 | ACUUCCCUCCUCUCUUCUG |
| | | U | | |
| siRNA 5185 | 5185 | AGAAGAGAGGAGGGAAGU | 11539 | AACUUCCCUCCUCUCUUCU |
| | | U | | |
| siRNA 5186 | 5186 | GAAGAGAGGAGGGAAGUU | 11540 | CAACUUCCCUCCUCUCUUC |
| | | G | | |
| siRNA 5187 | 5187 | AAGAGAGGAGGGAAGUUG | 11541 | ACAACUUCCCUCCUCUCUU |
| | | U | | |
| siRNA 5188 | 5188 | AGAGAGGAGGGAAGUUGU | 11542 | CACAACUUCCCUCCUCUCU |
| | | G | | |
| siRNA 5189 | 5189 | GAGAGGAGGGAAGUUGUG | 11543 | GCACAACUUCCCUCCUCUC |
| | | C | | |
| siRNA 5190 | 5190 | AGAGGAGGGAAGUUGUGC | 11544 | UGCACAACUUCCCUCCUCU |
| | | A | | |
| siRNA 5191 | 5191 | GAGGAGGGAAGUUGUGCA | 11545 | GUGCACAACUUCCCUCCUC |
| | | C | | |
| siRNA 5192 | 5192 | AGGAGGGAAGUUGUGCAC | 11546 | AGUGCACAACUUCCCUCCU |
| | | U | | |
| siRNA 5193 | 5193 | GGAGGGAAGUUGUGCACU | 11547 | GAGUGCACAACUUCCCUCC |
| | | C | | |
| siRNA 5194 | 5194 | GAGGGAAGUUGUGCACUC | 11548 | AGAGUGCACAACUUCCCUC |
| | | U | | |
| siRNA 5195 | 5195 | AGGGAAGUUGUGCACUCU | 11549 | CAGAGUGCACAACUUCCCU |
| | | G | | |
| siRNA 5196 | 5196 | GGGAAGUUGUGCACUCUG | 11550 | UCAGAGUGCACAACUUCCC |
| | | A | | |
| siRNA 5197 | 5197 | GGAAGUUGUGCACUCUGA | 11551 | UUCAGAGUGCACAACUUCC |
| | | A | | |
| siRNA 5198 | 5198 | GAAGUUGUGCACUCUGAA | 11552 | UUUCAGAGUGCACAACUUC |
| | | A | | |
| siRNA 5199 | 5199 | AAGUUGUGCACUCUGAAA | 11553 | UUUUCAGAGUGCACAACUU |
| | | A | | |
| siRNA 5200 | 5200 | AGUUGUGCACUCUGAAAA | 11554 | AUUUUCAGAGUGCACAACU |
| | | U | | |
| siRNA 5201 | 5201 | GUUGUGCACUCUGAAAAU | 11555 | CAUUUUCAGAGUGCACAAC |
| | | G | | |
| siRNA 5202 | 5202 | UUGUGCACUCUGAAAAUGC | 11556 | GCAUUUUCAGAGUGCACAA |
| siRNA 5203 | 5203 | UGUGCACUCUGAAAAUGCA | 11557 | UGCAUUUUCAGAGUGCACA |
| siRNA 5204 | 5204 | GUGCACUCUGAAAAUGCAU | 11558 | AUGCAUUUUCAGAGUGCAC |
| siRNA 5205 | 5205 | UGCACUCUGAAAAUGCAUG | 11559 | CAUGCAUUUUCAGAGUGCA |
| siRNA 5206 | 5206 | GCACUCUGAAAAUGCAUGA | 11560 | UCAUGCAUUUUCAGAGUGC |
| siRNA 5207 | 5207 | CACUCUGAAAAUGCAUGAA | 11561 | UUCAUGCAUUUUCAGAGU |
| | | | | G |
| siRNA 5208 | 5208 | ACUCUGAAAAUGCAUGAA | 11562 | UUUCAUGCAUUUUCAGAG |
| | | A | | U |
| siRNA 5209 | 5209 | CUCUGAAAAUGCAUGAAA | 11563 | UUUUCAUGCAUUUUCAGA |
| | | A | | G |
| siRNA 5210 | 5210 | UCUGAAAAUGCAUGAAAA | 11564 | UUUUUCAUGCAUUUUCAG |
| | | A | | A |
| siRNA 5211 | 5211 | CUGAAAAUGCAUGAAAAA | 11565 | GUUUUUCAUGCAUUUUCA |
| | | C | | G |
| siRNA 5212 | 5212 | UGAAAAUGCAUGAAAAAC | 11566 | UGUUUUUCAUGCAUUUUC |
| | | A | | A |
| siRNA 5213 | 5213 | GAAAAUGCAUGAAAAACA | 11567 | UUGUUUUUCAUGCAUUUU |
| | | A | | C |
| siRNA 5214 | 5214 | AAAAUGCAUGAAAAACAA | 11568 | UUUGUUUUUCAUGCAUUU |
| | | A | | U |
| siRNA 5215 | 5215 | AAAUGCAUGAAAAACAAA | 11569 | CUUUGUUUUUCAUGCAUU |
| | | G | | U |
| siRNA 5216 | 5216 | AAUGCAUGAAAAACAAAG | 11570 | CCUUUGUUUUUCAUGCAUU |
| | | G | | |
| siRNA 5217 | 5217 | AUGCAUGAAAAACAAAGG | 11571 | GCCUUUGUUUUUCAUGCAU |
| | | C | | |
| siRNA 5218 | 5218 | UGCAUGAAAAACAAAGGC | 11572 | UGCCUUUGUUUUUCAUGCA |
| | | A | | |
| siRNA 5219 | 5219 | GCAUGAAAAACAAAGGCA | 11573 | UUGCCUUUGUUUUUCAUGC |
| | | A | | |
| siRNA 5220 | 5220 | CAUGAAAAACAAAGGCAA | 11574 | UUUGCCUUUGUUUUUCAU |
| | | A | | G |
| siRNA 5221 | 5221 | AUGAAAAACAAAGGCAAA | 11575 | UUUUGCCUUUGUUUUUCA |
| | | A | | U |
| siRNA 5222 | 5222 | UGAAAAACAAAGGCAAAA | 11576 | UUUUUGCCUUUGUUUUUC |
| | | A | | A |
| siRNA 5223 | 5223 | GAAAAACAAAGGCAAAAA | 11577 | GUUUUUGCCUUUGUUUUU |
| | | C | | C |
| siRNA 5224 | 5224 | AAAAACAAAGGCAAAAAC | 11578 | AGUUUUUGCCUUUGUUUU |
| | | U | | U |
| siRNA 5225 | 5225 | AAAACAAAGGCAAAAACU | 11579 | UAGUUUUUGCCUUUGUUU |
| | | A | | U |
| siRNA 5226 | 5226 | AAACAAAGGCAAAAACUA | 11580 | CUAGUUUUUGCCUUUGUU |
| | | G | | U |
| siRNA 5227 | 5227 | AACAAAGGCAAAAACUAG | 11581 | ACUAGUUUUUGCCUUUGU |
| | | U | | U |
| siRNA 5228 | 5228 | ACAAAGGCAAAAACUAGU | 11582 | CACUAGUUUUUGCCUUUGU |
| | | G | | |
| siRNA 5229 | 5229 | CAAAGGCAAAAACUAGUG | 11583 | CCACUAGUUUUUGCCUUUG |
| | | G | | |
| siRNA 5230 | 5230 | AAAGGCAAAAACUAGUGG | 11584 | CCCACUAGUUUUUGCCUUU |
| | | G | | |
| siRNA 5231 | 5231 | AAGGCAAAAACUAGUGGG | 11585 | UCCCACUAGUUUUUGCCUU |
| | | A | | |
| siRNA 5232 | 5232 | AGGCAAAAACUAGUGGGA | 11586 | UUCCCACUAGUUUUUGCCU |
| | | A | | |
| siRNA 5233 | 5233 | GGCAAAAACUAGUGGGAA | 11587 | UUUCCCACUAGUUUUUGCC |
| | | A | | |
| siRNA 5234 | 5234 | GCAAAAACUAGUGGGAAA | 11588 | AUUUCCCACUAGUUUUUGC |
| | | U | | |
| siRNA 5235 | 5235 | CAAAAACUAGUGGGAAAU | 11589 | CAUUUCCCACUAGUUUUUG |
| | | G | | |
| siRNA 5236 | 5236 | AAAAACUAGUGGGAAAUG | 11590 | ACAUUUCCCACUAGUUUUU |
| | | U | | |
| siRNA 5237 | 5237 | AAAACUAGUGGGAAAUGU | 11591 | CACAUUUCCCACUAGUUUU |
| | | G | | |
| siRNA 5238 | 5238 | AAACUAGUGGGAAAUGUG | 11592 | ACACAUUUCCCACUAGUUU |
| | | U | | |
| siRNA 5239 | 5239 | AACUAGUGGGAAAUGUGU | 11593 | UACACAUUUCCCACUAGUU |
| | | A | | |
| siRNA 5240 | 5240 | ACUAGUGGGAAAUGUGUA | 11594 | CUACACAUUUCCCACUAGU |
| | | G | | |
| siRNA 5241 | 5241 | CUAGUGGGAAAUGUGUAG | 11595 | UCUACACAUUUCCCACUAG |
| | | A | | |
| siRNA 5242 | 5242 | UAGUGGGAAAUGUGUAGA | 11596 | UUCUACACAUUUCCCACUA |
| | | A | | |
| siRNA 5243 | 5243 | AGUGGGAAAUGUGUAGAA | 11597 | GUUCUACACAUUUCCCACU |
| | | C | | |
| siRNA 5244 | 5244 | GUGGGAAAUGUGUAGAAC | 11598 | AGUUCUACACAUUUCCCAC |
| | | U | | |
| siRNA 5245 | 5245 | UGGGAAAUGUGUAGAACU | 11599 | CAGUUCUACACAUUUCCCA |
| | | G | | |
| siRNA 5246 | 5246 | GGGAAAUGUGUAGAACUG | 11600 | ACAGUUCUACACAUUUCCC |
| | | U | | |
| siRNA 5247 | 5247 | GGAAAUGUGUAGAACUGU | 11601 | AACAGUUCUACACAUUUCC |
| | | U | | |
| siRNA 5248 | 5248 | GAAAUGUGUAGAACUGUU | 11602 | UAACAGUUCUACACAUUUC |
| | | A | | |
| siRNA 5249 | 5249 | AAAUGUGUAGAACUGUUA | 11603 | UUAACAGUUCUACACAUUU |
| | | A | | |
| siRNA 5250 | 5250 | AAUGUGUAGAACUGUUAA | 11604 | GUUAACAGUUCUACACAUU |
| | | C | | |
| siRNA 5251 | 5251 | AUGUGUAGAACUGUUAAC | 11605 | AGUUAACAGUUCUACACAU |
| | | U | | |
| siRNA 5252 | 5252 | UGUGUAGAACUGUUAACU | 11606 | CAGUUAACAGUUCUACACA |
| | | G | | |
| siRNA 5253 | 5253 | GUGUAGAACUGUUAACUG | 11607 | UCAGUUAACAGUUCUACAC |
| | | A | | |
| siRNA 5254 | 5254 | UGUAGAACUGUUAACUGA | 11608 | CUCAGUUAACAGUUCUACA |
| | | G | | |
| siRNA 5255 | 5255 | GUAGAACUGUUAACUGAG | 11609 | UCUCAGUUAACAGUUCUAC |
| | | A | | |
| siRNA 5256 | 5256 | UAGAACUGUUAACUGAGA | 11610 | AUCUCAGUUAACAGUUCUA |
| | | U | | |
| siRNA 5257 | 5257 | AGAACUGUUAACUGAGAU | 11611 | CAUCUCAGUUAACAGUUCU |
| | | G | | |
| siRNA 5258 | 5258 | GAACUGUUAACUGAGAUG | 11612 | CCAUCUCAGUUAACAGUUC |
| | | G | | |
| siRNA 5259 | 5259 | AACUGUUAACUGAGAUGG | 11613 | GCCAUCUCAGUUAACAGUU |
| | | C | | |
| siRNA 5260 | 5260 | ACUGUUAACUGAGAUGGC | 11614 | AGCCAUCUCAGUUAACAGU |
| | | U | | |
| siRNA 5261 | 5261 | CUGUUAACUGAGAUGGCU | 11615 | AAGCCAUCUCAGUUAACAG |
| | | U | | |
| siRNA 5262 | 5262 | UGUUAACUGAGAUGGCUU | 11616 | GAAGCCAUCUCAGUUAACA |
| | | C | | |
| siRNA 5263 | 5263 | GUUAACUGAGAUGGCUUC | 11617 | CGAAGCCAUCUCAGUUAAC |
| | | G | | |
| siRNA 5264 | 5264 | UUAACUGAGAUGGCUUCG | 11618 | UCGAAGCCAUCUCAGUUAA |
| | | A | | |
| siRNA 5265 | 5265 | UAACUGAGAUGGCUUCGA | 11619 | CUCGAAGCCAUCUCAGUUA |
| | | G | | |
| siRNA 5266 | 5266 | AACUGAGAUGGCUUCGAG | 11620 | ACUCGAAGCCAUCUCAGUU |
| | | U | | |
| siRNA 5267 | 5267 | ACUGAGAUGGCUUCGAGUC | 11621 | GACUCGAAGCCAUCUCAGU |
| siRNA 5268 | 5268 | CUGAGAUGGCUUCGAGUCU | 11622 | AGACUCGAAGCCAUCUCAG |
| siRNA 5269 | 5269 | UGAGAUGGCUUCGAGUCU | 11623 | AAGACUCGAAGCCAUCUCA |
| | | U | | |
| siRNA 5270 | 5270 | GAGAUGGCUUCGAGUCUUC | 11624 | GAAGACUCGAAGCCAUCUC |
| siRNA 5271 | 5271 | AGAUGGCUUCGAGUCUUCC | 11625 | GGAAGACUCGAAGCCAUCU |
| siRNA 5272 | 5272 | GAUGGCUUCGAGUCUUCCU | 11626 | AGGAAGACUCGAAGCCAUC |
| siRNA 5273 | 5273 | AUGGCUUCGAGUCUUCCUU | 11627 | AAGGAAGACUCGAAGCCAU |
| siRNA 5274 | 5274 | UGGCUUCGAGUCUUCCUUC | 11628 | GAAGGAAGACUCGAAGCCA |
| siRNA 5275 | 5275 | GGCUUCGAGUCUUCCUUCU | 11629 | AGAAGGAAGACUCGAAGCC |
| siRNA 5276 | 5276 | GCUUCGAGUCUUCCUUCUG | 11630 | CAGAAGGAAGACUCGAAGC |
| siRNA 5277 | 5277 | CUUCGAGUCUUCCUUCUGG | 11631 | CCAGAAGGAAGACUCGAAG |
| siRNA 5278 | 5278 | UUCGAGUCUUCCUUCUGGA | 11632 | UCCAGAAGGAAGACUCGAA |
| siRNA 5279 | 5279 | UCGAGUCUUCCUUCUGGAA | 11633 | UUCCAGAAGGAAGACUCGA |
| siRNA 5280 | 5280 | CGAGUCUUCCUUCUGGAAU | 11634 | AUUCCAGAAGGAAGACUCG |
| siRNA 5281 | 5281 | GAGUCUUCCUUCUGGAAUC | 11635 | GAUUCCAGAAGGAAGACUC |
| siRNA 5282 | 5282 | AGUCUUCCUUCUGGAAUCU | 11636 | AGAUUCCAGAAGGAAGAC |
| | | | | U |
| siRNA 5283 | 5283 | GUCUUCCUUCUGGAAUCUG | 11637 | CAGAUUCCAGAAGGAAGAC |
| siRNA 5284 | 5284 | UCUUCCUUCUGGAAUCUGU | 11638 | ACAGAUUCCAGAAGGAAG |
| | | | | A |
| siRNA 5285 | 5285 | CUUCCUUCUGGAAUCUGUU | 11639 | AACAGAUUCCAGAAGGAA |
| | | | | G |
| siRNA 5286 | 5286 | UUCCUUCUGGAAUCUGUUA | 11640 | UAACAGAUUCCAGAAGGA |
| | | | | A |
| siRNA 5287 | 5287 | UCCUUCUGGAAUCUGUUAA | 11641 | UUAACAGAUUCCAGAAGG |
| | | | | A |
| siRNA 5288 | 5288 | CCUUCUGGAAUCUGUUAAA | 11642 | UUUAACAGAUUCCAGAAG |
| | | | | G |
| siRNA 5289 | 5289 | CUUCUGGAAUCUGUUAAA | 11643 | AUUUAACAGAUUCCAGAA |
| | | U | | G |
| siRNA 5290 | 5290 | UUCUGGAAUCUGUUAAAU | 11644 | AAUUUAACAGAUUCCAGA |
| | | U | | A |
| siRNA 5291 | 5291 | UCUGGAAUCUGUUAAAUU | 11645 | AAAUUUAACAGAUUCCAG |
| | | U | | A |
| siRNA 5292 | 5292 | CUGGAAUCUGUUAAAUUU | 11646 | GAAAUUUAACAGAUUCCA |
| | | C | | G |
| siRNA 5293 | 5293 | UGGAAUCUGUUAAAUUUC | 11647 | UGAAAUUUAACAGAUUCC |
| | | A | | A |
| siRNA 5294 | 5294 | GGAAUCUGUUAAAUUUCA | 11648 | GUGAAAUUUAACAGAUUC |
| | | C | | C |
| siRNA 5295 | 5295 | GAAUCUGUUAAAUUUCAC | 11649 | UGUGAAAUUUAACAGAUU |
| | | A | | C |
| siRNA 5296 | 5296 | AAUCUGUUAAAUUUCACA | 11650 | UUGUGAAAUUUAACAGAU |
| | | A | | U |
| siRNA 5297 | 5297 | AUCUGUUAAAUUUCACAA | 11651 | UUUGUGAAAUUUAACAGA |
| | | A | | U |
| siRNA 5298 | 5298 | UCUGUUAAAUUUCACAAA | 11652 | CUUUGUGAAAUUUAACAG |
| | | G | | A |
| siRNA 5299 | 5299 | CUGUUAAAUUUCACAAAG | 11653 | ACUUUGUGAAAUUUAACA |
| | | U | | G |
| siRNA 5300 | 5300 | UGUUAAAUUUCACAAAGU | 11654 | GACUUUGUGAAAUUUAAC |
| | | C | | A |
| siRNA 5301 | 5301 | GUUAAAUUUCACAAAGUC | 11655 | UGACUUUGUGAAAUUUAA |
| | | A | | C |
| siRNA 5302 | 5302 | UUAAAUUUCACAAAGUCA | 11656 | AUGACUUUGUGAAAUUUA |
| | | U | | A |
| siRNA 5303 | 5303 | UAAAUUUCACAAAGUCAU | 11657 | CAUGACUUUGUGAAAUUU |
| | | G | | A |
| siRNA 5304 | 5304 | AAAUUUCACAAAGUCAUG | 11658 | UCAUGACUUUGUGAAAUU |
| | | A | | U |
| siRNA 5305 | 5305 | AAUUUCACAAAGUCAUGA | 11659 | CUCAUGACUUUGUGAAAU |
| | | G | | U |
| siRNA 5306 | 5306 | AUUUCACAAAGUCAUGAG | 11660 | CCUCAUGACUUUGUGAAAU |
| | | G | | |
| siRNA 5307 | 5307 | UUUCACAAAGUCAUGAGG | 11661 | CCCUCAUGACUUUGUGAAA |
| | | G | | |
| siRNA 5308 | 5308 | UUCACAAAGUCAUGAGGG | 11662 | ACCCUCAUGACUUUGUGAA |
| | | U | | |
| siRNA 5309 | 5309 | UCACAAAGUCAUGAGGGU | 11663 | UACCCUCAUGACUUUGUGA |
| | | A | | |
| siRNA 5310 | 5310 | CACAAAGUCAUGAGGGUA | 11664 | UUACCCUCAUGACUUUGUG |
| | | A | | |
| siRNA 5311 | 5311 | ACAAAGUCAUGAGGGUAA | 11665 | UUUACCCUCAUGACUUUGU |
| | | A | | |
| siRNA 5312 | 5312 | CAAAGUCAUGAGGGUAAA | 11666 | AUUUACCCUCAUGACUUUG |
| | | U | | |
| siRNA 5313 | 5313 | AAAGUCAUGAGGGUAAAU | 11667 | CAUUUACCCUCAUGACUUU |
| | | G | | |
| siRNA 5314 | 5314 | AAGUCAUGAGGGUAAAUG | 11668 | CCAUUUACCCUCAUGACUU |
| | | G | | |
| siRNA 5315 | 5315 | AGUCAUGAGGGUAAAUGG | 11669 | UCCAUUUACCCUCAUGACU |
| | | A | | |
| siRNA 5316 | 5316 | GUCAUGAGGGUAAAUGGA | 11670 | CUCCAUUUACCCUCAUGAC |
| | | G | | |
| siRNA 5317 | 5317 | UCAUGAGGGUAAAUGGAG | 11671 | UCUCCAUUUACCCUCAUGA |
| | | A | | |
| siRNA 5318 | 5318 | CAUGAGGGUAAAUGGAGA | 11672 | UUCUCCAUUUACCCUCAUG |
| | | A | | |
| siRNA 5319 | 5319 | AUGAGGGUAAAUGGAGAA | 11673 | UUUCUCCAUUUACCCUCAU |
| | | A | | |
| siRNA 5320 | 5320 | UGAGGGUAAAUGGAGAAA | 11674 | UUUUCUCCAUUUACCCUCA |
| | | A | | |
| siRNA 5321 | 5321 | GAGGGUAAAUGGAGAAAA | 11675 | AUUUUCUCCAUUUACCCUC |
| | | U | | |
| siRNA 5322 | 5322 | AGGGUAAAUGGAGAAAAU | 11676 | UAUUUUCUCCAUUUACCCU |
| | | A | | |
| siRNA 5323 | 5323 | GGGUAAAUGGAGAAAAUA | 11677 | AUAUUUUCUCCAUUUACCC |
| | | U | | |
| siRNA 5324 | 5324 | GGUAAAUGGAGAAAAUAU | 11678 | AAUAUUUUCUCCAUUUACC |
| | | U | | |
| siRNA 5325 | 5325 | GUAAAUGGAGAAAAUAUU | 11679 | AAAUAUUUUCUCCAUUUAC |
| | | U | | |
| siRNA 5326 | 5326 | UAAAUGGAGAAAAUAUUU | 11680 | GAAAUAUUUUCUCCAUUU |
| | | C | | A |
| siRNA 5327 | 5327 | AAAUGGAGAAAAUAUUUC | 11681 | AGAAAUAUUUUCUCCAUU |
| | | U | | U |
| siRNA 5328 | 5328 | AAUGGAGAAAAUAUUUCU | 11682 | CAGAAAUAUUUUCUCCAUU |
| | | G | | |
| siRNA 5329 | 5329 | AUGGAGAAAAUAUUUCUG | 11683 | CCAGAAAUAUUUUCUCCAU |
| | | G | | |
| siRNA 5330 | 5330 | UGGAGAAAAUAUUUCUGG | 11684 | CCCAGAAAUAUUUUCUCCA |
| | | G | | |
| siRNA 5331 | 5331 | GGAGAAAAUAUUUCUGGG | 11685 | UCCCAGAAAUAUUUUCUCC |
| | | A | | |
| siRNA 5332 | 5332 | GAGAAAAUAUUUCUGGGA | 11686 | AUCCCAGAAAUAUUUUCUC |
| | | U | | |
| siRNA 5333 | 5333 | AGAAAAUAUUUCUGGGAU | 11687 | AAUCCCAGAAAUAUUUUCU |
| | | U | | |
| siRNA 5334 | 5334 | GAAAAUAUUUCUGGGAUU | 11688 | UAAUCCCAGAAAUAUUUUC |
| | | A | | |
| siRNA 5335 | 5335 | AAAAUAUUUCUGGGAUUA | 11689 | GUAAUCCCAGAAAUAUUU |
| | | C | | U |
| siRNA 5336 | 5336 | AAAUAUUUCUGGGAUUAC | 11690 | UGUAAUCCCAGAAAUAUU |
| | | A | | U |
| siRNA 5337 | 5337 | AAUAUUUCUGGGAUUACA | 11691 | UUGUAAUCCCAGAAAUAU |
| | | A | | U |
| siRNA 5338 | 5338 | AUAUUUCUGGGAUUACAA | 11692 | AUUGUAAUCCCAGAAAUA |
| | | U | | U |
| siRNA 5339 | 5339 | UAUUUCUGGGAUUACAAU | 11693 | CAUUGUAAUCCCAGAAAUA |
| | | G | | |
| siRNA 5340 | 5340 | AUUUCUGGGAUUACAAUG | 11694 | UCAUUGUAAUCCCAGAAAU |
| | | A | | |
| siRNA 5341 | 5341 | UUUCUGGGAUUACAAUGA | 11695 | UUCAUUGUAAUCCCAGAAA |
| | | A | | |
| siRNA 5342 | 5342 | UUCUGGGAUUACAAUGAA | 11696 | AUUCAUUGUAAUCCCAGAA |
| | | U | | |
| siRNA 5343 | 5343 | UCUGGGAUUACAAUGAAU | 11697 | CAUUCAUUGUAAUCCCAGA |
| | | G | | |
| siRNA 5344 | 5344 | CUGGGAUUACAAUGAAUG | 11698 | ACAUUCAUUGUAAUCCCAG |
| | | U | | |
| siRNA 5345 | 5345 | UGGGAUUACAAUGAAUGU | 11699 | UACAUUCAUUGUAAUCCCA |
| | | A | | |
| siRNA 5346 | 5346 | GGGAUUACAAUGAAUGUA | 11700 | UUACAUUCAUUGUAAUCCC |
| | | A | | |
| siRNA 5347 | 5347 | GGAUUACAAUGAAUGUAA | 11701 | CUUACAUUCAUUGUAAUCC |
| | | G | | |
| siRNA 5348 | 5348 | GAUUACAAUGAAUGUAAG | 11702 | GCUUACAUUCAUUGUAAUC |
| | | C | | |
| siRNA 5349 | 5349 | AUUACAAUGAAUGUAAGC | 11703 | GGCUUACAUUCAUUGUAA |
| | | C | | U |
| siRNA 5350 | 5350 | UUACAAUGAAUGUAAGCCC | 11704 | GGGCUUACAUUCAUUGUA |
| | | | | A |
| siRNA 5351 | 5351 | UACAAUGAAUGUAAGCCCA | 11705 | UGGGCUUACAUUCAUUGU |
| | | | | A |
| siRNA 5352 | 5352 | ACAAUGAAUGUAAGCCCAA | 11706 | UUGGGCUUACAUUCAUUG |
| | | | | U |
| siRNA 5353 | 5353 | CAAUGAAUGUAAGCCCAAA | 11707 | UUUGGGCUUACAUUCAUU |
| | | | | G |
| siRNA 5354 | 5354 | AAUGAAUGUAAGCCCAAA | 11708 | AUUUGGGCUUACAUUCAU |
| | | U | | U |
| siRNA 5355 | 5355 | AUGAAUGUAAGCCCAAAU | 11709 | AAUUUGGGCUUACAUUCA |
| | | U | | U |
| siRNA 5356 | 5356 | UGAAUGUAAGCCCAAAUU | 11710 | CAAUUUGGGCUUACAUUCA |
| | | G | | |
| siRNA 5357 | 5357 | GAAUGUAAGCCCAAAUUG | 11711 | ACAAUUUGGGCUUACAUUC |
| | | U | | |
| siRNA 5358 | 5358 | AAUGUAAGCCCAAAUUGU | 11712 | CACAAUUUGGGCUUACAUU |
| | | G | | |
| siRNA 5359 | 5359 | AUGUAAGCCCAAAUUGUG | 11713 | CCACAAUUUGGGCUUACAU |
| | | G | | |
| siRNA 5360 | 5360 | UGUAAGCCCAAAUUGUGG | 11714 | UCCACAAUUUGGGCUUACA |
| | | A | | |
| siRNA 5361 | 5361 | GUAAGCCCAAAUUGUGGA | 11715 | UUCCACAAUUUGGGCUUAC |
| | | A | | |
| siRNA 5362 | 5362 | UAAGCCCAAAUUGUGGAA | 11716 | AUUCCACAAUUUGGGCUUA |
| | | U | | |
| siRNA 5363 | 5363 | AAGCCCAAAUUGUGGAAU | 11717 | AAUUCCACAAUUUGGGCUU |
| | | U | | |
| siRNA 5364 | 5364 | AGCCCAAAUUGUGGAAUU | 11718 | CAAUUCCACAAUUUGGGCU |
| | | G | | |
| siRNA 5365 | 5365 | GCCCAAAUUGUGGAAUUGC | 11719 | GCAAUUCCACAAUUUGGGC |
| siRNA 5366 | 5366 | CCCAAAUUGUGGAAUUGCC | 11720 | GGCAAUUCCACAAUUUGGG |
| siRNA 5367 | 5367 | CCAAAUUGUGGAAUUGCCA | 11721 | UGGCAAUUCCACAAUUUGG |
| siRNA 5368 | 5368 | CAAAUUGUGGAAUUGCCA | 11722 | CUGGCAAUUCCACAAUUUG |
| | | G | | |
| siRNA 5369 | 5369 | AAAUUGUGGAAUUGCCAG | 11723 | ACUGGCAAUUCCACAAUUU |
| | | U | | |
| siRNA 5370 | 5370 | AAUUGUGGAAUUGCCAGU | 11724 | UACUGGCAAUUCCACAAUU |
| | | A | | |
| siRNA 5371 | 5371 | AUUGUGGAAUUGCCAGUA | 11725 | UUACUGGCAAUUCCACAAU |
| | | A | | |
| siRNA 5372 | 5372 | UUGUGGAAUUGCCAGUAA | 11726 | GUUACUGGCAAUUCCACAA |
| | | C | | |
| siRNA 5373 | 5373 | UGUGGAAUUGCCAGUAACC | 11727 | GGUUACUGGCAAUUCCACA |
| siRNA 5374 | 5374 | GUGGAAUUGCCAGUAACCU | 11728 | AGGUUACUGGCAAUUCCAC |
| siRNA 5375 | 5375 | UGGAAUUGCCAGUAACCUG | 11729 | CAGGUUACUGGCAAUUCCA |
| siRNA 5376 | 5376 | GGAAUUGCCAGUAACCUGG | 11730 | CCAGGUUACUGGCAAUUCC |
| siRNA 5377 | 5377 | GAAUUGCCAGUAACCUGGA | 11731 | UCCAGGUUACUGGCAAUUC |
| siRNA 5378 | 5378 | AAUUGCCAGUAACCUGGAU | 11732 | AUCCAGGUUACUGGCAAUU |
| siRNA 5379 | 5379 | AUUGCCAGUAACCUGGAUG | 11733 | CAUCCAGGUUACUGGCAAU |
| siRNA 5380 | 5380 | UUGCCAGUAACCUGGAUGG | 11734 | CCAUCCAGGUUACUGGCAA |
| siRNA 5381 | 5381 | UGCCAGUAACCUGGAUGGG | 11735 | CCCAUCCAGGUUACUGGCA |
| siRNA 5382 | 5382 | GCCAGUAACCUGGAUGGGG | 11736 | CCCCAUCCAGGUUACUGGC |
| siRNA 5383 | 5383 | CCAGUAACCUGGAUGGGGA | 11737 | UCCCCAUCCAGGUUACUGG |
| siRNA 5384 | 5384 | CAGUAACCUGGAUGGGGA | 11738 | UUCCCCAUCCAGGUUACUG |
| | | A | | |
| siRNA 5385 | 5385 | AGUAACCUGGAUGGGGAA | 11739 | UUUCCCCAUCCAGGUUACU |
| | | A | | |
| siRNA 5386 | 5386 | GUAACCUGGAUGGGGAAA | 11740 | UUUUCCCCAUCCAGGUUAC |
| | | A | | |
| siRNA 5387 | 5387 | UAACCUGGAUGGGGAAAA | 11741 | CUUUUCCCCAUCCAGGUUA |
| | | G | | |
| siRNA 5388 | 5388 | AACCUGGAUGGGGAAAAG | 11742 | GCUUUUCCCCAUCCAGGUU |
| | | C | | |
| siRNA 5389 | 5389 | ACCUGGAUGGGGAAAAGC | 11743 | UGCUUUUCCCCAUCCAGGU |
| | | A | | |
| siRNA 5390 | 5390 | CCUGGAUGGGGAAAAGCA | 11744 | AUGCUUUUCCCCAUCCAGG |
| | | U | | |
| siRNA 5391 | 5391 | CUGGAUGGGGAAAAGCAU | 11745 | AAUGCUUUUCCCCAUCCAG |
| | | U | | |
| siRNA 5392 | 5392 | UGGAUGGGGAAAAGCAUU | 11746 | AAAUGCUUUUCCCCAUCCA |
| | | U | | |
| siRNA 5393 | 5393 | GGAUGGGGAAAAGCAUUU | 11747 | GAAAUGCUUUUCCCCAUCC |
| | | C | | |
| siRNA 5394 | 5394 | GAUGGGGAAAAGCAUUUC | 11748 | GGAAAUGCUUUUCCCCAUC |
| | | C | | |
| siRNA 5395 | 5395 | AUGGGGAAAAGCAUUUCCC | 11749 | GGGAAAUGCUUUUCCCCAU |
| siRNA 5396 | 5396 | UGGGGAAAAGCAUUUCCCA | 11750 | UGGGAAAUGCUUUUCCCCA |
| siRNA 5397 | 5397 | GGGGAAAAGCAUUUCCCAU | 11751 | AUGGGAAAUGCUUUUCCCC |
| siRNA 5398 | 5398 | GGGAAAAGCAUUUCCCAUA | 11752 | UAUGGGAAAUGCUUUUCCC |
| siRNA 5399 | 5399 | GGAAAAGCAUUUCCCAUAG | 11753 | CUAUGGGAAAUGCUUUUCC |
| siRNA 5400 | 5400 | GAAAAGCAUUUCCCAUAGC | 11754 | GCUAUGGGAAAUGCUUUU |
| | | | | C |
| siRNA 5401 | 5401 | AAAAGCAUUUCCCAUAGCA | 11755 | UGCUAUGGGAAAUGCUUU |
| | | | | U |
| siRNA 5402 | 5402 | AAAGCAUUUCCCAUAGCAC | 11756 | GUGCUAUGGGAAAUGCUU |
| | | | | U |
| siRNA 5403 | 5403 | AAGCAUUUCCCAUAGCACU | 11757 | AGUGCUAUGGGAAAUGCU |
| | | | | U |
| siRNA 5404 | 5404 | AGCAUUUCCCAUAGCACUC | 11758 | GAGUGCUAUGGGAAAUGC |
| | | | | U |
| siRNA 5405 | 5405 | GCAUUUCCCAUAGCACUCC | 11759 | GGAGUGCUAUGGGAAAUG |
| | | | | C |
| siRNA 5406 | 5406 | CAUUUCCCAUAGCACUCCA | 11760 | UGGAGUGCUAUGGGAAAU |
| | | | | G |
| siRNA 5407 | 5407 | AUUUCCCAUAGCACUCCAU | 11761 | AUGGAGUGCUAUGGGAAA |
| | | | | U |
| siRNA 5408 | 5408 | UUUCCCAUAGCACUCCAUG | 11762 | CAUGGAGUGCUAUGGGAA |
| | | | | A |
| siRNA 5409 | 5409 | UUCCCAUAGCACUCCAUGU | 11763 | ACAUGGAGUGCUAUGGGA |
| | | | | A |
| siRNA 5410 | 5410 | UCCCAUAGCACUCCAUGUA | 11764 | UACAUGGAGUGCUAUGGG |
| | | | | A |
| siRNA 5411 | 5411 | CCCAUAGCACUCCAUGUAA | 11765 | UUACAUGGAGUGCUAUGG |
| | | | | G |
| siRNA 5412 | 5412 | CCAUAGCACUCCAUGUAAU | 11766 | AUUACAUGGAGUGCUAUG |
| | | | | G |
| siRNA 5413 | 5413 | CAUAGCACUCCAUGUAAUA | 11767 | UAUUACAUGGAGUGCUAU |
| | | | | G |
| siRNA 5414 | 5414 | AUAGCACUCCAUGUAAUAU | 11768 | AUAUUACAUGGAGUGCUA |
| | | | | U |
| siRNA 5415 | 5415 | UAGCACUCCAUGUAAUAUG | 11769 | CAUAUUACAUGGAGUGCU |
| | | | | A |
| siRNA 5416 | 5416 | AGCACUCCAUGUAAUAUGA | 11770 | UCAUAUUACAUGGAGUGC |
| | | | | U |
| siRNA 5417 | 5417 | GCACUCCAUGUAAUAUGAG | 11771 | CUCAUAUUACAUGGAGUGC |
| siRNA 5418 | 5418 | CACUCCAUGUAAUAUGAGU | 11772 | ACUCAUAUUACAUGGAGU |
| | | | | G |
| siRNA 5419 | 5419 | ACUCCAUGUAAUAUGAGU | 11773 | CACUCAUAUUACAUGGAGU |
| | | G | | |
| siRNA 5420 | 5420 | CUCCAUGUAAUAUGAGUGC | 11774 | GCACUCAUAUUACAUGGAG |
| siRNA 5421 | 5421 | UCCAUGUAAUAUGAGUGC | 11775 | AGCACUCAUAUUACAUGGA |
| | | U | | |
| siRNA 5422 | 5422 | CCAUGUAAUAUGAGUGCUC | 11776 | GAGCACUCAUAUUACAUGG |
| siRNA 5423 | 5423 | CAUGUAAUAUGAGUGCUC | 11777 | AGAGCACUCAUAUUACAUG |
| | | U | | |
| siRNA 5424 | 5424 | AUGUAAUAUGAGUGCUCU | 11778 | CAGAGCACUCAUAUUACAU |
| | | G | | |
| siRNA 5425 | 5425 | UGUAAUAUGAGUGCUCUG | 11779 | ACAGAGCACUCAUAUUACA |
| | | U | | |
| siRNA 5426 | 5426 | GUAAUAUGAGUGCUCUGU | 11780 | CACAGAGCACUCAUAUUAC |
| | | G | | |
| siRNA 5427 | 5427 | UAAUAUGAGUGCUCUGUG | 11781 | UCACAGAGCACUCAUAUUA |
| | | A | | |
| siRNA 5428 | 5428 | AAUAUGAGUGCUCUGUGA | 11782 | CUCACAGAGCACUCAUAUU |
| | | G | | |
| siRNA 5429 | 5429 | AUAUGAGUGCUCUGUGAG | 11783 | UCUCACAGAGCACUCAUAU |
| | | A | | |
| siRNA 5430 | 5430 | UAUGAGUGCUCUGUGAGA | 11784 | AUCUCACAGAGCACUCAUA |
| | | U | | |
| siRNA 5431 | 5431 | AUGAGUGCUCUGUGAGAU | 11785 | CAUCUCACAGAGCACUCAU |
| | | G | | |
| siRNA 5432 | 5432 | UGAGUGCUCUGUGAGAUG | 11786 | ACAUCUCACAGAGCACUCA |
| | | U | | |
| siRNA 5433 | 5433 | GAGUGCUCUGUGAGAUGU | 11787 | AACAUCUCACAGAGCACUC |
| | | U | | |
| siRNA 5434 | 5434 | AGUGCUCUGUGAGAUGUU | 11788 | GAACAUCUCACAGAGCACU |
| | | C | | |
| siRNA 5435 | 5435 | GUGCUCUGUGAGAUGUUC | 11789 | UGAACAUCUCACAGAGCAC |
| | | A | | |
| siRNA 5436 | 5436 | UGCUCUGUGAGAUGUUCA | 11790 | AUGAACAUCUCACAGAGCA |
| | | U | | |
| siRNA 5437 | 5437 | GCUCUGUGAGAUGUUCAUC | 11791 | GAUGAACAUCUCACAGAGC |
| siRNA 5438 | 5438 | CUCUGUGAGAUGUUCAUCA | 11792 | UGAUGAACAUCUCACAGAG |
| siRNA 5439 | 5439 | UCUGUGAGAUGUUCAUCA | 11793 | CUGAUGAACAUCUCACAGA |
| | | G | | |
| siRNA 5440 | 5440 | CUGUGAGAUGUUCAUCAG | 11794 | ACUGAUGAACAUCUCACAG |
| | | U | | |
| siRNA 5441 | 5441 | UGUGAGAUGUUCAUCAGU | 11795 | CACUGAUGAACAUCUCACA |
| | | G | | |
| siRNA 5442 | 5442 | GUGAGAUGUUCAUCAGUG | 11796 | ACACUGAUGAACAUCUCAC |
| | | U | | |
| siRNA 5443 | 5443 | UGAGAUGUUCAUCAGUGU | 11797 | AACACUGAUGAACAUCUCA |
| | | U | | |
| siRNA 5444 | 5444 | GAGAUGUUCAUCAGUGUU | 11798 | AAACACUGAUGAACAUCUC |
| | | U | | |
| siRNA 5445 | 5445 | AGAUGUUCAUCAGUGUUU | 11799 | AAAACACUGAUGAACAUCU |
| | | U | | |
| siRNA 5446 | 5446 | GAUGUUCAUCAGUGUUUU | 11800 | UAAAACACUGAUGAACAUC |
| | | A | | |
| siRNA 5447 | 5447 | AUGUUCAUCAGUGUUUUA | 11801 | AUAAAACACUGAUGAACA |
| | | U | | U |
| siRNA 5448 | 5448 | UGUUCAUCAGUGUUUUAU | 11802 | UAUAAAACACUGAUGAAC |
| | | A | | A |
| siRNA 5449 | 5449 | GUUCAUCAGUGUUUUAUA | 11803 | CUAUAAAACACUGAUGAAC |
| | | G | | |
| siRNA 5450 | 5450 | UUCAUCAGUGUUUUAUAG | 11804 | UCUAUAAAACACUGAUGA |
| | | A | | A |
| siRNA 5451 | 5451 | UCAUCAGUGUUUUAUAGA | 11805 | UUCUAUAAAACACUGAUG |
| | | A | | A |
| siRNA 5452 | 5452 | CAUCAGUGUUUUAUAGAA | 11806 | UUUCUAUAAAACACUGAU |
| | | A | | G |
| siRNA 5453 | 5453 | AUCAGUGUUUUAUAGAAA | 11807 | AUUUCUAUAAAACACUGA |
| | | U | | U |
| siRNA 5454 | 5454 | UCAGUGUUUUAUAGAAAU | 11808 | CAUUUCUAUAAAACACUGA |
| | | G | | |
| siRNA 5455 | 5455 | CAGUGUUUUAUAGAAAUG | 11809 | CCAUUUCUAUAAAACACUG |
| | | G | | |
| siRNA 5456 | 5456 | AGUGUUUUAUAGAAAUGG | 11810 | ACCAUUUCUAUAAAACACU |
| | | U | | |
| siRNA 5457 | 5457 | GUGUUUUAUAGAAAUGGU | 11811 | CACCAUUUCUAUAAAACAC |
| | | G | | |
| siRNA 5458 | 5458 | UGUUUUAUAGAAAUGGUG | 11812 | ACACCAUUUCUAUAAAACA |
| | | U | | |
| siRNA 5459 | 5459 | GUUUUAUAGAAAUGGUGU | 11813 | AACACCAUUUCUAUAAAAC |
| | | U | | |
| siRNA 5460 | 5460 | UUUUAUAGAAAUGGUGUU | 11814 | CAACACCAUUUCUAUAAAA |
| | | G | | |
| siRNA 5461 | 5461 | UUUAUAGAAAUGGUGUUG | 11815 | GCAACACCAUUUCUAUAAA |
| | | C | | |
| siRNA 5462 | 5462 | UUAUAGAAAUGGUGUUGC | 11816 | AGCAACACCAUUUCUAUAA |
| | | U | | |
| siRNA 5463 | 5463 | UAUAGAAAUGGUGUUGCU | 11817 | CAGCAACACCAUUUCUAUA |
| | | G | | |
| siRNA 5464 | 5464 | AUAGAAAUGGUGUUGCUG | 11818 | CCAGCAACACCAUUUCUAU |
| | | G | | |
| siRNA 5465 | 5465 | UAGAAAUGGUGUUGCUGG | 11819 | CCCAGCAACACCAUUUCUA |
| | | G | | |
| siRNA 5466 | 5466 | AGAAAUGGUGUUGCUGGG | 11820 | UCCCAGCAACACCAUUUCU |
| | | A | | |
| siRNA 5467 | 5467 | GAAAUGGUGUUGCUGGGA | 11821 | UUCCCAGCAACACCAUUUC |
| | | A | | |
| siRNA 5468 | 5468 | AAAUGGUGUUGCUGGGAA | 11822 | UUUCCCAGCAACACCAUUU |
| | | A | | |
| siRNA 5469 | 5469 | AAUGGUGUUGCUGGGAAA | 11823 | GUUUCCCAGCAACACCAUU |
| | | C | | |
| siRNA 5470 | 5470 | AUGGUGUUGCUGGGAAAC | 11824 | GGUUUCCCAGCAACACCAU |
| | | C | | |
| siRNA 5471 | 5471 | UGGUGUUGCUGGGAAACC | 11825 | UGGUUUCCCAGCAACACCA |
| | | A | | |
| siRNA 5472 | 5472 | GGUGUUGCUGGGAAACCA | 11826 | UUGGUUUCCCAGCAACACC |
| | | A | | |
| siRNA 5473 | 5473 | GUGUUGCUGGGAAACCAA | 11827 | CUUGGUUUCCCAGCAACAC |
| | | G | | |
| siRNA 5474 | 5474 | UGUUGCUGGGAAACCAAG | 11828 | ACUUGGUUUCCCAGCAACA |
| | | U | | |
| siRNA 5475 | 5475 | GUUGCUGGGAAACCAAGU | 11829 | AACUUGGUUUCCCAGCAAC |
| | | U | | |
| siRNA 5476 | 5476 | UUGCUGGGAAACCAAGUU | 11830 | AAACUUGGUUUCCCAGCAA |
| | | U | | |
| siRNA 5477 | 5477 | UGCUGGGAAACCAAGUUU | 11831 | CAAACUUGGUUUCCCAGCA |
| | | G | | |
| siRNA 5478 | 5478 | GCUGGGAAACCAAGUUUGC | 11832 | GCAAACUUGGUUUCCCAGC |
| siRNA 5479 | 5479 | CUGGGAAACCAAGUUUGCA | 11833 | UGCAAACUUGGUUUCCCAG |
| siRNA 5480 | 5480 | UGGGAAACCAAGUUUGCAC | 11834 | GUGCAAACUUGGUUUCCCA |
| siRNA 5481 | 5481 | GGGAAACCAAGUUUGCACC | 11835 | GGUGCAAACUUGGUUUCCC |
| siRNA 5482 | 5482 | GGAAACCAAGUUUGCACCU | 11836 | AGGUGCAAACUUGGUUUCC |
| siRNA 5483 | 5483 | GAAACCAAGUUUGCACCUG | 11837 | CAGGUGCAAACUUGGUUUC |
| siRNA 5484 | 5484 | AAACCAAGUUUGCACCUGG | 11838 | CCAGGUGCAAACUUGGUUU |
| siRNA 5485 | 5485 | AACCAAGUUUGCACCUGGA | 11839 | UCCAGGUGCAAACUUGGUU |
| siRNA 5486 | 5486 | ACCAAGUUUGCACCUGGAA | 11840 | UUCCAGGUGCAAACUUGGU |
| siRNA 5487 | 5487 | CCAAGUUUGCACCUGGAAA | 11841 | UUUCCAGGUGCAAACUUGG |
| siRNA 5488 | 5488 | CAAGUUUGCACCUGGAAAC | 11842 | GUUUCCAGGUGCAAACUUG |
| siRNA 5489 | 5489 | AAGUUUGCACCUGGAAACU | 11843 | AGUUUCCAGGUGCAAACUU |
| siRNA 5490 | 5490 | AGUUUGCACCUGGAAACUU | 11844 | AAGUUUCCAGGUGCAAACU |
| siRNA 5491 | 5491 | GUUUGCACCUGGAAACUUA | 11845 | UAAGUUUCCAGGUGCAAAC |
| siRNA 5492 | 5492 | UUUGCACCUGGAAACUUAC | 11846 | GUAAGUUUCCAGGUGCAA |
| | | | | A |
| siRNA 5493 | 5493 | UUGCACCUGGAAACUUACA | 11847 | UGUAAGUUUCCAGGUGCA |
| | | | | A |
| siRNA 5494 | 5494 | UGCACCUGGAAACUUACAA | 11848 | UUGUAAGUUUCCAGGUGC |
| | | | | A |
| siRNA 5495 | 5495 | GCACCUGGAAACUUACAAU | 11849 | AUUGUAAGUUUCCAGGUG |
| | | | | C |
| siRNA 5496 | 5496 | CACCUGGAAACUUACAAUG | 11850 | CAUUGUAAGUUUCCAGGU |
| | | | | G |
| siRNA 5497 | 5497 | ACCUGGAAACUUACAAUGC | 11851 | GCAUUGUAAGUUUCCAGG |
| | | | | U |
| siRNA 5498 | 5498 | CCUGGAAACUUACAAUGCA | 11852 | UGCAUUGUAAGUUUCCAG |
| | | | | G |
| siRNA 5499 | 5499 | CUGGAAACUUACAAUGCAC | 11853 | GUGCAUUGUAAGUUUCCA |
| | | | | G |
| siRNA 5500 | 5500 | UGGAAACUUACAAUGCACU | 11854 | AGUGCAUUGUAAGUUUCC |
| | | | | A |
| siRNA 5501 | 5501 | GGAAACUUACAAUGCACUU | 11855 | AAGUGCAUUGUAAGUUUC |
| | | | | C |
| siRNA 5502 | 5502 | GAAACUUACAAUGCACUUU | 11856 | AAAGUGCAUUGUAAGUUU |
| | | | | C |
| siRNA 5503 | 5503 | AAACUUACAAUGCACUUUA | 11857 | UAAAGUGCAUUGUAAGUU |
| | | | | U |
| siRNA 5504 | 5504 | AACUUACAAUGCACUUUAG | 11858 | CUAAAGUGCAUUGUAAGU |
| | | | | U |
| siRNA 5505 | 5505 | ACUUACAAUGCACUUUAGC | 11859 | GCUAAAGUGCAUUGUAAG |
| | | | | U |
| siRNA 5506 | 5506 | CUUACAAUGCACUUUAGCG | 11860 | CGCUAAAGUGCAUUGUAA |
| | | | | G |
| siRNA 5507 | 5507 | UUACAAUGCACUUUAGCGC | 11861 | GCGCUAAAGUGCAUUGUA |
| | | | | A |
| siRNA 5508 | 5508 | UACAAUGCACUUUAGCGCA | 11862 | UGCGCUAAAGUGCAUUGU |
| | | | | A |
| siRNA 5509 | 5509 | ACAAUGCACUUUAGCGCAG | 11863 | CUGCGCUAAAGUGCAUUGU |
| siRNA 5510 | 5510 | CAAUGCACUUUAGCGCAGU | 11864 | ACUGCGCUAAAGUGCAUUG |
| siRNA 5511 | 5511 | AAUGCACUUUAGCGCAGUA | 11865 | UACUGCGCUAAAGUGCAUU |
| siRNA 5512 | 5512 | AUGCACUUUAGCGCAGUAA | 11866 | UUACUGCGCUAAAGUGCAU |
| siRNA 5513 | 5513 | UGCACUUUAGCGCAGUAAG | 11867 | CUUACUGCGCUAAAGUGCA |
| siRNA 5514 | 5514 | GCACUUUAGCGCAGUAAGG | 11868 | CCUUACUGCGCUAAAGUGC |
| siRNA 5515 | 5515 | CACUUUAGCGCAGUAAGGG | 11869 | CCCUUACUGCGCUAAAGUG |
| siRNA 5516 | 5516 | ACUUUAGCGCAGUAAGGGC | 11870 | GCCCUUACUGCGCUAAAGU |
| siRNA 5517 | 5517 | CUUUAGCGCAGUAAGGGCU | 11871 | AGCCCUUACUGCGCUAAAG |
| siRNA 5518 | 5518 | UUUAGCGCAGUAAGGGCU | 11872 | AAGCCCUUACUGCGCUAAA |
| | | U | | |
| siRNA 5519 | 5519 | UUAGCGCAGUAAGGGCUU | 11873 | CAAGCCCUUACUGCGCUAA |
| | | G | | |
| siRNA 5520 | 5520 | UAGCGCAGUAAGGGCUUG | 11874 | CCAAGCCCUUACUGCGCUA |
| | | G | | |
| siRNA 5521 | 5521 | AGCGCAGUAAGGGCUUGGC | 11875 | GCCAAGCCCUUACUGCGCU |
| siRNA 5522 | 5522 | GCGCAGUAAGGGCUUGGCA | 11876 | UGCCAAGCCCUUACUGCGC |
| siRNA 5523 | 5523 | CGCAGUAAGGGCUUGGCAU | 11877 | AUGCCAAGCCCUUACUGCG |
| siRNA 5524 | 5524 | GCAGUAAGGGCUUGGCAUC | 11878 | GAUGCCAAGCCCUUACUGC |
| siRNA 5525 | 5525 | CAGUAAGGGCUUGGCAUCC | 11879 | GGAUGCCAAGCCCUUACUG |
| siRNA 5526 | 5526 | AGUAAGGGCUUGGCAUCCG | 11880 | CGGAUGCCAAGCCCUUACU |
| siRNA 5527 | 5527 | GUAAGGGCUUGGCAUCCGG | 11881 | CCGGAUGCCAAGCCCUUAC |
| siRNA 5528 | 5528 | UAAGGGCUUGGCAUCCGGU | 11882 | ACCGGAUGCCAAGCCCUUA |
| siRNA 5529 | 5529 | AAGGGCUUGGCAUCCGGUA | 11883 | UACCGGAUGCCAAGCCCUU |
| siRNA 5530 | 5530 | AGGGCUUGGCAUCCGGUAG | 11884 | CUACCGGAUGCCAAGCCCU |
| siRNA 5531 | 5531 | GGGCUUGGCAUCCGGUAGU | 11885 | ACUACCGGAUGCCAAGCCC |
| siRNA 5532 | 5532 | GGCUUGGCAUCCGGUAGUG | 11886 | CACUACCGGAUGCCAAGCC |
| siRNA 5533 | 5533 | GCUUGGCAUCCGGUAGUGA | 11887 | UCACUACCGGAUGCCAAGC |
| siRNA 5534 | 5534 | CUUGGCAUCCGGUAGUGAA | 11888 | UUCACUACCGGAUGCCAAG |
| siRNA 5535 | 5535 | UUGGCAUCCGGUAGUGAA | 11889 | UUUCACUACOGGAUGCCAA |
| | | A | | |
| siRNA 5536 | 5536 | UGGCAUCCGGUAGUGAAA | 11890 | UUUUCACUACCGGAUGCCA |
| | | A | | |
| siRNA 5537 | 5537 | GGCAUCCGGUAGUGAAAA | | |
| | | A | 11891 | UUUUUCACUACCGGAUGCC |
| siRNA 5538 | 5538 | GCAUCCGGUAGUGAAAAAC | 11892 | GUUUUUCACUACCGGAUGC |
| siRNA 5539 | 5539 | CAUCCGGUAGUGAAAAACU | 11893 | AGUUUUUCACUACCGGAUG |
| siRNA 5540 | 5540 | AUCCGGUAGUGAAAAACU | 11894 | CAGUUUUUCACUACCGGAU |
| | | G | | |
| siRNA 5541 | 5541 | UCCGGUAGUGAAAAACUG | 11895 | ACAGUUUUUCACUACCGGA |
| | | U | | |
| siRNA 5542 | 5542 | CCGGUAGUGAAAAACUGUC | 11896 | GACAGUUUUUCACUACCGG |
| siRNA 5543 | 5543 | CGGUAGUGAAAAACUGUC | 11897 | AGACAGUUUUUCACUACCG |
| | | U | | |
| siRNA 5544 | 5544 | GGUAGUGAAAAACUGUCU | 11898 | UAGACAGUUUUUCACUACO |
| | | A | | |
| siRNA 5545 | 5545 | GUAGUGAAAAACUGUCUA | 11899 | UUAGACAGUUUUUCACUAC |
| | | A | | |
| siRNA 5546 | 5546 | UAGUGAAAAACUGUCUAA | 11900 | GUUAGACAGUUUUUCACU |
| | | C | | A |
| siRNA 5547 | 5547 | AGUGAAAAACUGUCUAACC | 11901 | GGUUAGACAGUUUUUCAC |
| | | | | U |
| siRNA 5548 | 5548 | GUGAAAAACUGUCUAACCC | 11902 | GGGUUAGACAGUUUUUCA |
| | | | | C |
| siRNA 5549 | 5549 | UGAAAAACUGUCUAACCCA | 11903 | UGGGUUAGACAGUUUUUC |
| | | | | A |
| siRNA 5550 | 5550 | GAAAAACUGUCUAACCCAG | 11904 | CUGGGUUAGACAGUUUUU |
| | | | | C |
| siRNA 5551 | 5551 | AAAAACUGUCUAACCCAGC | 11905 | GCUGGGUUAGACAGUUUU |
| | | | | U |
| siRNA 5552 | 5552 | AAAACUGUCUAACCCAGCA | 11906 | UGCUGGGUUAGACAGUUU |
| | | | | U |
| siRNA 5553 | 5553 | AAACUGUCUAACCCAGCAU | 11907 | AUGCUGGGUUAGACAGUU |
| | | | | U |
| siRNA 5554 | 5554 | AACUGUCUAACCCAGCAUU | 11908 | AAUGCUGGGUUAGACAGU |
| | | | | U |
| siRNA 5555 | 5555 | ACUGUCUAACCCAGCAUUG | 11909 | CAAUGCUGGGUUAGACAG |
| | | | | U |
| siRNA 5556 | 5556 | CUGUCUAACCCAGCAUUGC | 11910 | GCAAUGCUGGGUUAGACA |
| | | | | G |
| siRNA 5557 | 5557 | UGUCUAACCCAGCAUUGCC | 11911 | GGCAAUGCUGGGUUAGAC |
| | | | | A |
| siRNA 5558 | 5558 | GUCUAACCCAGCAUUGCCC | 11912 | GGGCAAUGCUGGGUUAGA |
| | | | | C |
| siRNA 5559 | 5559 | UCUAACCCAGCAUUGCCCA | 11913 | UGGGCAAUGCUGGGUUAG |
| | | | | A |
| siRNA 5560 | 5560 | CUAACCCAGCAUUGCCCAA | 11914 | UUGGGCAAUGCUGGGUUA |
| | | | | G |
| siRNA 5561 | 5561 | UAACCCAGCAUUGCCCAAA | 11915 | UUUGGGCAAUGCUGGGUU |
| | | | | A |
| siRNA 5562 | 5562 | AACCCAGCAUUGCCCAAAC | 11916 | GUUUGGGCAAUGCUGGGU |
| | | | | U |
| siRNA 5563 | 5563 | ACCCAGCAUUGCCCAAACU | 11917 | AGUUUGGGCAAUGCUGGG |
| | | | | U |
| siRNA 5564 | 5564 | CCCAGCAUUGCCCAAACUA | 11918 | UAGUUUGGGCAAUGCUGG |
| | | | | G |
| siRNA 5565 | 5565 | CCAGCAUUGCCCAAACUAU | 11919 | AUAGUUUGGGCAAUGCUG |
| | | | | G |
| siRNA 5566 | 5566 | CAGCAUUGCCCAAACUAUU | 11920 | AAUAGUUUGGGCAAUGCU |
| | | | | G |
| siRNA 5567 | 5567 | AGCAUUGCCCAAACUAUUU | 11921 | AAAUAGUUUGGGCAAUGC |
| | | | | U |
| siRNA 5568 | 5568 | GCAUUGCCCAAACUAUUUU | 11922 | AAAAUAGUUUGGGCAAUG |
| | | | | C |
| siRNA 5569 | 5569 | CAUUGCCCAAACUAUUUUG | 11923 | CAAAAUAGUUUGGGCAAU |
| | | | | G |
| siRNA 5570 | 5570 | AUUGCCCAAACUAUUUUGA | 11924 | UCAAAAUAGUUUGGGCAA |
| | | | | U |
| siRNA 5571 | 5571 | UUGCCCAAACUAUUUUGAC | 11925 | GUCAAAAUAGUUUGGGCA |
| | | | | A |
| siRNA 5572 | 5572 | UGCCCAAACUAUUUUGACA | 11926 | UGUCAAAAUAGUUUGGGC |
| | | | | A |
| siRNA 5573 | 5573 | GCCCAAACUAUUUUGACAC | 11927 | GUGUCAAAAUAGUUUGGG |
| | | | | C |
| siRNA 5574 | 5574 | CCCAAACUAUUUUGACACC | 11928 | GGUGUCAAAAUAGUUUGG |
| | | | | G |
| siRNA 5575 | 5575 | CCAAACUAUUUUGACACCA | 11929 | UGGUGUCAAAAUAGUUUG |
| | | | | G |
| siRNA 5576 | 5576 | CAAACUAUUUUGACACCAG | 11930 | CUGGUGUCAAAAUAGUUU |
| | | | | G |
| siRNA 5577 | 5577 | AAACUAUUUUGACACCAGG | 11931 | CCUGGUGUCAAAAUAGUU |
| | | | | U |
| siRNA 5578 | 5578 | AACUAUUUUGACACCAGGA | 11932 | UCCUGGUGUCAAAAUAGU |
| | | | | U |
| siRNA 5579 | 5579 | ACUAUUUUGACACCAGGAC | 11933 | GUCCUGGUGUCAAAAUAG |
| | | | | U |
| siRNA 5580 | 5580 | CUAUUUUGACACCAGGACC | 11934 | GGUCCUGGUGUCAAAAUA |
| | | | | G |
| siRNA 5581 | 5581 | UAUUUUGACACCAGGACCU | 11935 | AGGUCCUGGUGUCAAAAU |
| | | | | A |
| siRNA 5582 | 5582 | AUUUUGACACCAGGACCUU | 11936 | AAGGUCCUGGUGUCAAAA |
| | | | | U |
| siRNA 5583 | 5583 | UUUUGACACCAGGACCUUU | 11937 | AAAGGUCCUGGUGUCAAA |
| | | | | A |
| siRNA 5584 | 5584 | UUUGACACCAGGACCUUUU | 11938 | AAAAGGUCCUGGUGUCAA |
| | | | | A |
| siRNA 5585 | 5585 | UUGACACCAGGACCUUUUU | 11939 | AAAAAGGUCCUGGUGUCA |
| | | | | A |
| siRNA 5586 | 5586 | UGACACCAGGACCUUUUUC | 11940 | GAAAAAGGUCCUGGUGUC |
| | | | | A |
| siRNA 5587 | 5587 | GACACCAGGACCUUUUUCU | 11941 | AGAAAAAGGUCCUGGUGU |
| | | | | C |
| siRNA 5588 | 5588 | ACACCAGGACCUUUUUCUC | 11942 | GAGAAAAAGGUCCUGGUG |
| | | | | U |
| siRNA 5589 | 5589 | CACCAGGACCUUUUUCUCC | 11943 | GGAGAAAAAGGUCCUGGU |
| | | | | G |
| siRNA 5590 | 5590 | ACCAGGACCUUUUUCUCCU | 11944 | AGGAGAAAAAGGUCCUGG |
| | | | | U |
| siRNA 5591 | 5591 | CCAGGACCUUUUUCUCCUU | 11945 | AAGGAGAAAAAGGUCCUG |
| | | | | G |
| siRNA 5592 | 5592 | CAGGACCUUUUUCUCCUUU | 11946 | AAAGGAGAAAAAGGUCCU |
| | | | | G |
| siRNA 5593 | 5593 | AGGACCUUUUUCUCCUUUG | 11947 | CAAAGGAGAAAAAGGUCC |
| | | | | U |
| siRNA 5594 | 5594 | GGACCUUUUUCUCCUUUGG | 11948 | CCAAAGGAGAAAAAGGUCC |
| siRNA 5595 | 5595 | GACCUUUUUCUCCUUUGGG | 11949 | CCCAAAGGAGAAAAAGGUC |
| siRNA 5596 | 5596 | ACCUUUUUCUCCUUUGGGA | 11950 | UCCCAAAGGAGAAAAAGG |
| | | | | U |
| siRNA 5597 | 5597 | CCUUUUUCUCCUUUGGGAU | 11951 | AUCCCAAAGGAGAAAAAG |
| | | | | G |
| siRNA 5598 | 5598 | CUUUUUCUCCUUUGGGAUA | 11952 | UAUCCCAAAGGAGAAAAA |
| | | | | G |
| siRNA 5599 | 5599 | UUUUUCUCCUUUGGGAUAC | 11953 | GUAUCCCAAAGGAGAAAA |
| | | | | A |
| siRNA 5600 | 5600 | UUUUCUCCUUUGGGAUACU | 11954 | AGUAUCCCAAAGGAGAAA |
| | | | | A |
| siRNA 5601 | 5601 | UUUCUCCUUUGGGAUACUU | 11955 | AAGUAUCCCAAAGGAGAA |
| | | | | A |
| siRNA 5602 | 5602 | UUCUCCUUUGGGAUACUUA | 11956 | UAAGUAUCCCAAAGGAGA |
| | | | | A |
| siRNA 5603 | 5603 | UCUCCUUUGGGAUACUUAU | 11957 | AUAAGUAUCCCAAAGGAG |
| | | | | A |
| siRNA 5604 | 5604 | CUCCUUUGGGAUACUUAUG | 11958 | CAUAAGUAUCCCAAAGGAG |
| siRNA 5605 | 5605 | UCCUUUGGGAUACUUAUG | 11959 | UCAUAAGUAUCCCAAAGGA |
| | | A | | |
| siRNA 5606 | 5606 | CCUUUGGGAUACUUAUGA | 11960 | UUCAUAAGUAUCCCAAAGG |
| | | A | | |
| siRNA 5607 | 5607 | CUUUGGGAUACUUAUGAA | 11961 | GUUCAUAAGUAUCCCAAAG |
| | | C | | |
| siRNA 5608 | 5608 | UUUGGGAUACUUAUGAAC | 11962 | GGUUCAUAAGUAUCCCAAA |
| | | C | | |
| siRNA 5609 | 5609 | UUGGGAUACUUAUGAACC | 11963 | AGGUUCAUAAGUAUCCCAA |
| | | U | | |
| siRNA 5610 | 5610 | UGGGAUACUUAUGAACCUC | 11964 | GAGGUUCAUAAGUAUCCCA |
| siRNA 5611 | 5611 | GGGAUACUUAUGAACCUCU | 11965 | AGAGGUUCAUAAGUAUCCC |
| siRNA 5612 | 5612 | GGAUACUUAUGAACCUCUC | 11966 | GAGAGGUUCAUAAGUAUC |
| | | | | C |
| siRNA 5613 | 5613 | GAUACUUAUGAACCUCUCA | 11967 | UGAGAGGUUCAUAAGUAU |
| | | | | C |
| siRNA 5614 | 5614 | AUACUUAUGAACCUCUCAC | 11968 | GUGAGAGGUUCAUAAGUA |
| | | | | U |
| siRNA 5615 | 5615 | UACUUAUGAACCUCUCACU | 11969 | AGUGAGAGGUUCAUAAGU |
| | | | | A |
| siRNA 5616 | 5616 | ACUUAUGAACCUCUCACUA | 11970 | UAGUGAGAGGUUCAUAAG |
| | | | | U |
| siRNA 5617 | 5617 | CUUAUGAACCUCUCACUAA | 11971 | UUAGUGAGAGGUUCAUAA |
| | | | | G |
| siRNA 5618 | 5618 | UUAUGAACCUCUCACUAAU | 11972 | AUUAGUGAGAGGUUCAUA |
| | | | | A |
| siRNA 5619 | 5619 | UAUGAACCUCUCACUAAUG | 11973 | CAUUAGUGAGAGGUUCAU |
| | | | | A |
| siRNA 5620 | 5620 | AUGAACCUCUCACUAAUGU | 11974 | ACAUUAGUGAGAGGUUCA |
| | | | | U |
| siRNA 5621 | 5621 | UGAACCUCUCACUAAUGUC | 11975 | GACAUUAGUGAGAGGUUC |
| | | | | A |
| siRNA 5622 | 5622 | GAACCUCUCACUAAUGUCC | 11976 | GGACAUUAGUGAGAGGUU |
| | | | | C |
| siRNA 5623 | 5623 | AACCUCUCACUAAUGUCCU | 11977 | AGGACAUUAGUGAGAGGU |
| | | | | U |
| siRNA 5624 | 5624 | ACCUCUCACUAAUGUCCUG | 11978 | CAGGACAUUAGUGAGAGG |
| | | | | U |
| siRNA 5625 | 5625 | CCUCUCACUAAUGUCCUGU | 11979 | ACAGGACAUUAGUGAGAG |
| | | | | G |
| siRNA 5626 | 5626 | CUCUCACUAAUGUCCUGUG | 11980 | CACAGGACAUUAGUGAGA |
| | | | | G |
| siRNA 5627 | 5627 | UCUCACUAAUGUCCUGUGG | 11981 | CCACAGGACAUUAGUGAGA |
| siRNA 5628 | 5628 | CUCACUAAUGUCCUGUGGA | 11982 | UCCACAGGACAUUAGUGAG |
| siRNA 5629 | 5629 | UCACUAAUGUCCUGUGGAG | 11983 | CUCCACAGGACAUUAGUGA |
| siRNA 5630 | 5630 | CACUAAUGUCCUGUGGAGA | 11984 | UCUCCACAGGACAUUAGUG |
| siRNA 5631 | 5631 | ACUAAUGUCCUGUGGAGA | 11985 | UUCUCCACAGGACAUUAGU |
| | | A | | |
| siRNA 5632 | 5632 | CUAAUGUCCUGUGGAGAAC | 11986 | GUUCUCCACAGGACAUUAG |
| siRNA 5633 | 5633 | UAAUGUCCUGUGGAGAAC | 11987 | UGUUCUCCACAGGACAUUA |
| | | A | | |
| siRNA 5634 | 5634 | AAUGUCCUGUGGAGAACA | 11988 | AUGUUCUCCACAGGACAUU |
| | | U | | |
| siRNA 5635 | 5635 | AUGUCCUGUGGAGAACAU | 11989 | AAUGUUCUCCACAGGACAU |
| | | U | | |
| siRNA 5636 | 5636 | UGUCCUGUGGAGAACAUU | 11990 | AAAUGUUCUCCACAGGACA |
| | | U | | |
| siRNA 5637 | 5637 | GUCCUGUGGAGAACAUUU | 11991 | AAAAUGUUCUCCACAGGAC |
| | | U | | |
| siRNA 5638 | 5638 | UCCUGUGGAGAACAUUUU | 11992 | CAAAAUGUUCUCCACAGGA |
| | | G | | |
| siRNA 5639 | 5639 | CCUGUGGAGAACAUUUUG | 11993 | CCAAAAUGUUCUCCACAGG |
| | | G | | |
| siRNA 5640 | 5640 | CUGUGGAGAACAUUUUGG | 11994 | CCCAAAAUGUUCUCCACAG |
| | | G | | |
| siRNA 5641 | 5641 | UGUGGAGAACAUUUUGGG | 11995 | UCCCAAAAUGUUCUCCACA |
| | | A | | |
| siRNA 5642 | 5642 | GUGGAGAACAUUUUGGGA | 11996 | UUCCCAAAAUGUUCUCCAC |
| | | A | | |
| siRNA 5643 | 5643 | UGGAGAACAUUUUGGGAA | 11997 | UUUCCCAAAAUGUUCUCCA |
| | | A | | |
| siRNA 5644 | 5644 | GGAGAACAUUUUGGGAAA | 11998 | GUUUCCCAAAAUGUUCUCC |
| | | C | | |
| siRNA 5645 | 5645 | GAGAACAUUUUGGGAAAC | 11999 | UGUUUCCCAAAAUGUUCUC |
| | | A | | |
| siRNA 5646 | 5646 | AGAACAUUUUGGGAAACA | 12000 | GUGUUUCCCAAAAUGUUCU |
| | | C | | |
| siRNA 5647 | 5647 | GAACAUUUUGGGAAACAC | 12001 | AGUGUUUCCCAAAAUGUUC |
| | | U | | |
| siRNA 5648 | 5648 | AACAUUUUGGGAAACACU | 12002 | UAGUGUUUCCCAAAAUGU |
| | | A | | U |
| siRNA 5649 | 5649 | ACAUUUUGGGAAACACUA | 12003 | AUAGUGUUUCCCAAAAUG |
| | | U | | U |
| siRNA 5650 | 5650 | CAUUUUGGGAAACACUAU | 12004 | CAUAGUGUUUCCCAAAAUG |
| | | G | | |
| siRNA 5651 | 5651 | AUUUUGGGAAACACUAUG | 12005 | ACAUAGUGUUUCCCAAAAU |
| | | U | | |
| siRNA 5652 | 5652 | UUUUGGGAAACACUAUGU | 12006 | AACAUAGUGUUUCCCAAAA |
| | | U | | |
| siRNA 5653 | 5653 | UUUGGGAAACACUAUGUU | 12007 | UAACAUAGUGUUUCCCAAA |
| | | A | | |
| siRNA 5654 | 5654 | UUGGGAAACACUAUGUUA | 12008 | CUAACAUAGUGUUUCCCAA |
| | | G | | |
| siRNA 5655 | 5655 | UGGGAAACACUAUGUUAG | 12009 | UCUAACAUAGUGUUUCCCA |
| | | A | | |
| siRNA 5656 | 5656 | GGGAAACACUAUGUUAGA | 12010 | AUCUAACAUAGUGUUUCCC |
| | | U | | |
| siRNA 5657 | 5657 | GGAAACACUAUGUUAGAU | 12011 | UAUCUAACAUAGUGUUUCC |
| | | A | | |
| siRNA 5658 | 5658 | GAAACACUAUGUUAGAUA | 12012 | CUAUCUAACAUAGUGUUUC |
| | | G | | |
| siRNA 5659 | 5659 | AAACACUAUGUUAGAUAG | 12013 | ACUAUCUAACAUAGUGUU |
| | | U | | U |
| siRNA 5660 | 5660 | AACACUAUGUUAGAUAGU | 12014 | AACUAUCUAACAUAGUGU |
| | | U | | U |
| siRNA 5661 | 5661 | ACACUAUGUUAGAUAGUU | 12015 | GAACUAUCUAACAUAGUG |
| | | C | | U |
| siRNA 5662 | 5662 | CACUAUGUUAGAUAGUUC | 12016 | AGAACUAUCUAACAUAGU |
| | | U | | G |
| siRNA 5663 | 5663 | ACUAUGUUAGAUAGUUCU | 12017 | AAGAACUAUCUAACAUAG |
| | | U | | U |
| siRNA 5664 | 5664 | CUAUGUUAGAUAGUUCUU | 12018 | AAAGAACUAUCUAACAUA |
| | | U | | G |
| siRNA 5665 | 5665 | UAUGUUAGAUAGUUCUUU | 12019 | UAAAGAACUAUCUAACAU |
| | | A | | A |
| siRNA 5666 | 5666 | AUGUUAGAUAGUUCUUUA | 12020 | UUAAAGAACUAUCUAACA |
| | | A | | U |
| siRNA 5667 | 5667 | UGUUAGAUAGUUCUUUAA | 12021 | CUUAAAGAACUAUCUAACA |
| | | G | | |
| siRNA 5668 | 5668 | GUUAGAUAGUUCUUUAAG | 12022 | CCUUAAAGAACUAUCUAAC |
| | | G | | |
| siRNA 5669 | 5669 | UUAGAUAGUUCUUUAAGG | 12023 | UCCUUAAAGAACUAUCUAA |
| | | A | | |
| siRNA 5670 | 5670 | UAGAUAGUUCUUUAAGGA | 12024 | CUCCUUAAAGAACUAUCUA |
| | | G | | |
| siRNA 5671 | 5671 | AGAUAGUUCUUUAAGGAG | 12025 | UCUCCUUAAAGAACUAUCU |
| | | A | | |
| siRNA 5672 | 5672 | GAUAGUUCUUUAAGGAGA | 12026 | GUCUCCUUAAAGAACUAUC |
| | | C | | |
| siRNA 5673 | 5673 | AUAGUUCUUUAAGGAGAC | 12027 | UGUCUCCUUAAAGAACUAU |
| | | A | | |
| siRNA 5674 | 5674 | UAGUUCUUUAAGGAGACA | 12028 | UUGUCUCCUUAAAGAACUA |
| | | A | | |
| siRNA 5675 | 5675 | AGUUCUUUAAGGAGACAA | 12029 | UUUGUCUCCUUAAAGAACU |
| | | A | | |
| siRNA 5676 | 5676 | GUUCUUUAAGGAGACAAA | 12030 | UUUUGUCUCCUUAAAGAAC |
| | | A | | |
| siRNA 5677 | 5677 | UUCUUUAAGGAGACAAAA | 12031 | GUUUUGUCUCCUUAAAGA |
| | | C | | A |
| siRNA 5678 | 5678 | UCUUUAAGGAGACAAAAC | 12032 | CGUUUUGUCUCCUUAAAGA |
| | | G | | |
| siRNA 5679 | 5679 | CUUUAAGGAGACAAAACG | 12033 | CCGUUUUGUCUCCUUAAAG |
| | | G | | |
| siRNA 5680 | 5680 | UUUAAGGAGACAAAACGG | 12034 | ACCGUUUUGUCUCCUUAAA |
| | | U | | |
| siRNA 5681 | 5681 | UUAAGGAGACAAAACGGU | 12035 | UACCGUUUUGUCUCCUUAA |
| | | A | | |
| siRNA 5682 | 5682 | UAAGGAGACAAAACGGUA | 12036 | UUACCGUUUUGUCUCCUUA |
| | | A | | |
| siRNA 5683 | 5683 | AAGGAGACAAAACGGUAA | 12037 | AUUACCGUUUUGUCUCCUU |
| | | U | | |
| siRNA 5684 | 5684 | AGGAGACAAAACGGUAAU | 12038 | CAUUACCGUUUUGUCUCCU |
| | | G | | |
| siRNA 5685 | 5685 | GGAGACAAAACGGUAAUG | 12039 | UCAUUACCGUUUUGUCUCC |
| | | A | | |
| siRNA 5686 | 5686 | GAGACAAAACGGUAAUGA | 12040 | UUCAUUACCGUUUUGUCUC |
| | | A | | |
| siRNA 5687 | 5687 | AGACAAAACGGUAAUGAA | 12041 | GUUCAUUACCGUUUUGUCU |
| | | C | | |
| siRNA 5688 | 5688 | GACAAAACGGUAAUGAAC | 12042 | UGUUCAUUACCGUUUUGUC |
| | | A | | |
| siRNA 5689 | 5689 | ACAAAACGGUAAUGAACA | 12043 | CUGUUCAUUACCGUUUUGU |
| | | G | | |
| siRNA 5690 | 5690 | CAAAACGGUAAUGAACAG | 12044 | UCUGUUCAUUACCGUUUUG |
| | | A | | |
| siRNA 5691 | 5691 | AAAACGGUAAUGAACAGA | 12045 | AUCUGUUCAUUACCGUUUU |
| | | U | | |
| siRNA 5692 | 5692 | AAACGGUAAUGAACAGAU | 12046 | UAUCUGUUCAUUACCGUUU |
| | | A | | |
| siRNA 5693 | 5693 | AACGGUAAUGAACAGAUA | 12047 | CUAUCUGUUCAUUACCGUU |
| | | G | | |
| siRNA 5694 | 5694 | ACGGUAAUGAACAGAUAG | 12048 | GCUAUCUGUUCAUUACCGU |
| | | C | | |
| siRNA 5695 | 5695 | CGGUAAUGAACAGAUAGC | 12049 | UGCUAUCUGUUCAUUACCG |
| | | A | | |
| siRNA 5696 | 5696 | GGUAAUGAACAGAUAGCA | 12050 | GUGCUAUCUGUUCAUUACC |
| | | C | | |
| siRNA 5697 | 5697 | GUAAUGAACAGAUAGCAC | 12051 | AGUGCUAUCUGUUCAUUAC |
| | | U | | |
| siRNA 5698 | 5698 | UAAUGAACAGAUAGCACU | 12052 | CAGUGCUAUCUGUUCAUUA |
| | | G | | |
| siRNA 5699 | 5699 | AAUGAACAGAUAGCACUG | 12053 | CCAGUGCUAUCUGUUCAUU |
| | | G | | |
| siRNA 5700 | 5700 | AUGAACAGAUAGCACUGG | 12054 | CCCAGUGCUAUCUGUUCAU |
| | | G | | |
| siRNA 5701 | 5701 | UGAACAGAUAGCACUGGG | 12055 | CCCCAGUGCUAUCUGUUCA |
| | | G | | |
| siRNA 5702 | 5702 | GAACAGAUAGCACUGGGGC | 12056 | GCCCCAGUGCUAUCUGUUC |
| siRNA 5703 | 5703 | AACAGAUAGCACUGGGGCA | 12057 | UGCCCCAGUGCUAUCUGUU |
| siRNA 5704 | 5704 | ACAGAUAGCACUGGGGCAG | 12058 | CUGCCCCAGUGCUAUCUGU |
| siRNA 5705 | 5705 | CAGAUAGCACUGGGGCAGA | 12059 | UCUGCCCCAGUGCUAUCUG |
| siRNA 5706 | 5706 | AGAUAGCACUGGGGCAGA | 12060 | UUCUGCCCCAGUGCUAUCU |
| | | A | | |
| siRNA 5707 | 5707 | GAUAGCACUGGGGCAGAA | 12061 | AUUCUGCCCCAGUGCUAUC |
| | | U | | |
| siRNA 5708 | 5708 | AUAGCACUGGGGCAGAAU | 12062 | UAUUCUGCCCCAGUGCUAU |
| | | A | | |
| siRNA 5709 | 5709 | UAGCACUGGGGCAGAAUA | 12063 | AUAUUCUGCCCCAGUGCUA |
| | | U | | |
| siRNA 5710 | 5710 | AGCACUGGGGCAGAAUAU | 12064 | CAUAUUCUGCCCCAGUGCU |
| | | G | | |
| siRNA 5711 | 5711 | GCACUGGGGCAGAAUAUGC | 12065 | GCAUAUUCUGCCCCAGUGC |
| siRNA 5712 | 5712 | CACUGGGGCAGAAUAUGCA | 12066 | UGCAUAUUCUGCCCCAGUG |
| siRNA 5713 | 5713 | ACUGGGGCAGAAUAUGCA | 12067 | AUGCAUAUUCUGCCCCAGU |
| | | U | | |
| siRNA 5714 | 5714 | CUGGGGCAGAAUAUGCAU | 12068 | CAUGCAUAUUCUGCCCCAG |
| | | G | | |
| siRNA 5715 | 5715 | UGGGGCAGAAUAUGCAUG | 12069 | GCAUGCAUAUUCUGCCCCA |
| | | C | | |
| siRNA 5716 | 5716 | GGGGCAGAAUAUGCAUGC | 12070 | UGCAUGCAUAUUCUGCCCC |
| | | A | | |
| siRNA 5717 | 5717 | GGGCAGAAUAUGCAUGCA | 12071 | AUGCAUGCAUAUUCUGCCC |
| | | U | | |
| siRNA 5718 | 5718 | GGCAGAAUAUGCAUGCAU | 12072 | AAUGCAUGCAUAUUCUGCC |
| | | U | | |
| siRNA 5719 | 5719 | GCAGAAUAUGCAUGCAUU | 12073 | AAAUGCAUGCAUAUUCUGC |
| | | U | | |
| siRNA 5720 | 5720 | CAGAAUAUGCAUGCAUUU | 12074 | AAAAUGCAUGCAUAUUCU |
| | | U | | G |
| siRNA 5721 | 5721 | AGAAUAUGCAUGCAUUUU | 12075 | CAAAAUGCAUGCAUAUUCU |
| | | G | | |
| siRNA 5722 | 5722 | GAAUAUGCAUGCAUUUUG | 12076 | ACAAAAUGCAUGCAUAUUC |
| | | U | | |
| siRNA 5723 | 5723 | AAUAUGCAUGCAUUUUGU | 12077 | UACAAAAUGCAUGCAUAU |
| | | A | | U |
| siRNA 5724 | 5724 | AUAUGCAUGCAUUUUGUA | 12078 | UUACAAAAUGCAUGCAUA |
| | | A | | U |
| siRNA 5725 | 5725 | UAUGCAUGCAUUUUGUAA | 12079 | GUUACAAAAUGCAUGCAU |
| | | C | | A |
| siRNA 5726 | 5726 | AUGCAUGCAUUUUGUAAC | 12080 | CGUUACAAAAUGCAUGCAU |
| | | G | | |
| siRNA 5727 | 5727 | UGCAUGCAUUUUGUAACG | 12081 | ACGUUACAAAAUGCAUGCA |
| | | U | | |
| siRNA 5728 | 5728 | GCAUGCAUUUUGUAACGUC | 12082 | GACGUUACAAAAUGCAUGC |
| siRNA 5729 | 5729 | CAUGCAUUUUGUAACGUCC | 12083 | GGACGUUACAAAAUGCAU |
| | | | | G |
| siRNA 5730 | 5730 | AUGCAUUUUGUAACGUCCA | 12084 | UGGACGUUACAAAAUGCA |
| | | | | U |
| siRNA 5731 | 5731 | UGCAUUUUGUAACGUCCAG | 12085 | CUGGACGUUACAAAAUGCA |
| siRNA 5732 | 5732 | GCAUUUUGUAACGUCCAGU | 12086 | ACUGGACGUUACAAAAUGC |
| siRNA 5733 | 5733 | CAUUUUGUAACGUCCAGUG | 12087 | CACUGGACGUUACAAAAUG |
| siRNA 5734 | 5734 | AUUUUGUAACGUCCAGUG | 12088 | ACACUGGACGUUACAAAAU |
| | | U | | |
| siRNA 5735 | 5735 | UUUUGUAACGUCCAGUGU | 12089 | CACACUGGACGUUACAAAA |
| | | G | | |
| siRNA 5736 | 5736 | UUUGUAACGUCCAGUGUG | 12090 | CCACACUGGACGUUACAAA |
| | | G | | |
| siRNA 5737 | 5737 | UUGUAACGUCCAGUGUGGC | 12091 | GCCACACUGGACGUUACAA |
| siRNA 5738 | 5738 | UGUAACGUCCAGUGUGGCG | 12092 | CGCCACACUGGACGUUACA |
| siRNA 5739 | 5739 | GUAACGUCCAGUGUGGCGU | 12093 | ACGCCACACUGGACGUUAC |
| siRNA 5740 | 5740 | UAACGUCCAGUGUGGCGUU | 12094 | AACGCCACACUGGACGUUA |
| siRNA 5741 | 5741 | AACGUCCAGUGUGGCGUUG | 12095 | CAACGCCACACUGGACGUU |
| siRNA 5742 | 5742 | ACGUCCAGUGUGGCGUUGA | 12096 | UCAACGCCACACUGGACGU |
| siRNA 5743 | 5743 | CGUCCAGUGUGGCGUUGAA | 12097 | UUCAACGCCACACUGGACG |
| siRNA 5744 | 5744 | GUCCAGUGUGGCGUUGAA | 12098 | AUUCAACGCCACACUGGAC |
| | | U | | |
| siRNA 5745 | 5745 | UCCAGUGUGGCGUUGAAU | 12099 | UAUUCAACGCCACACUGGA |
| | | A | | |
| siRNA 5746 | 5746 | CCAGUGUGGCGUUGAAUA | 12100 | CUAUUCAACGCCACACUGG |
| | | G | | |
| siRNA 5747 | 5747 | CAGUGUGGCGUUGAAUAG | 12101 | UCUAUUCAACGCCACACUG |
| | | A | | |
| siRNA 5748 | 5748 | AGUGUGGCGUUGAAUAGA | 12102 | AUCUAUUCAACGCCACACU |
| | | U | | |
| siRNA 5749 | 5749 | GUGUGGCGUUGAAUAGAU | 12103 | CAUCUAUUCAACGCCACAC |
| | | G | | |
| siRNA 5750 | 5750 | UGUGGCGUUGAAUAGAUG | 12104 | ACAUCUAUUCAACGCCACA |
| | | U | | |
| siRNA 5751 | 5751 | GUGGCGUUGAAUAGAUGU | 12105 | CACAUCUAUUCAACGCCAC |
| | | G | | |
| siRNA 5752 | 5752 | UGGCGUUGAAUAGAUGUG | 12106 | ACACAUCUAUUCAACGCCA |
| | | U | | |
| siRNA 5753 | 5753 | GGCGUUGAAUAGAUGUGU | 12107 | UACACAUCUAUUCAACGCC |
| | | A | | |
| siRNA 5754 | 5754 | GCGUUGAAUAGAUGUGUA | 12108 | AUACACAUCUAUUCAACGC |
| | | U | | |
| siRNA 5755 | 5755 | CGUUGAAUAGAUGUGUAU | 12109 | AAUACACAUCUAUUCAACG |
| | | U | | |
| siRNA 5756 | 5756 | GUUGAAUAGAUGUGUAUU | 12110 | AAAUACACAUCUAUUCAAC |
| | | U | | |
| siRNA 5757 | 5757 | UUGAAUAGAUGUGUAUUU | 12111 | GAAAUACACAUCUAUUCAA |
| | | C | | |
| siRNA 5758 | 5758 | UGAAUAGAUGUGUAUUUC | 12112 | GGAAAUACACAUCUAUUCA |
| | | C | | |
| siRNA 5759 | 5759 | GAAUAGAUGUGUAUUUCC | 12113 | AGGAAAUACACAUCUAUUC |
| | | U | | |
| siRNA 5760 | 5760 | AAUAGAUGUGUAUUUCCU | 12114 | GAGGAAAUACACAUCUAU |
| | | C | | U |
| siRNA 5761 | 5761 | AUAGAUGUGUAUUUCCUCC | 12115 | GGAGGAAAUACACAUCUA |
| | | | | U |
| siRNA 5762 | 5762 | UAGAUGUGUAUUUCCUCCC | 12116 | GGGAGGAAAUACACAUCU |
| | | | | A |
| siRNA 5763 | 5763 | AGAUGUGUAUUUCCUCCCC | 12117 | GGGGAGGAAAUACACAUC |
| | | | | U |
| siRNA 5764 | 5764 | GAUGUGUAUUUCCUCCCCU | 12118 | AGGGGAGGAAAUACACAU |
| | | | | C |
| siRNA 5765 | 5765 | AUGUGUAUUUCCUCCCCUG | 12119 | CAGGGGAGGAAAUACACA |
| | | | | U |
| siRNA 5766 | 5766 | UGUGUAUUUCCUCCCCUGC | 12120 | GCAGGGGAGGAAAUACAC |
| | | | | A |
| siRNA 5767 | 5767 | GUGUAUUUCCUCCCCUGCA | 12121 | UGCAGGGGAGGAAAUACA |
| | | | | C |
| siRNA 5768 | 5768 | UGUAUUUCCUCCCCUGCAG | 12122 | CUGCAGGGGAGGAAAUAC |
| | | | | A |
| siRNA 5769 | 5769 | GUAUUUCCUCCCCUGCAGA | 12123 | UCUGCAGGGGAGGAAAUA |
| | | | | C |
| siRNA 5770 | 5770 | UAUUUCCUCCCCUGCAGAA | 12124 | UUCUGCAGGGGAGGAAAU |
| | | | | A |
| siRNA 5771 | 5771 | AUUUCCUCCCCUGCAGAAA | 12125 | UUUCUGCAGGGGAGGAAA |
| | | | | U |
| siRNA 5772 | 5772 | UUUCCUCCCCUGCAGAAAA | 12126 | UUUUCUGCAGGGGAGGAA |
| | | | | A |
| siRNA 5773 | 5773 | UUCCUCCCCUGCAGAAAAU | 12127 | AUUUUCUGCAGGGGAGGA |
| | | | | A |
| siRNA 5774 | 5774 | UCCUCCCCUGCAGAAAAUA | 12128 | UAUUUUCUGCAGGGGAGG |
| | | | | A |
| siRNA 5775 | 5775 | CCUCCCCUGCAGAAAAUAA | 12129 | UUAUUUUCUGCAGGGGAG |
| | | | | G |
| siRNA 5776 | 5776 | CUCCCCUGCAGAAAAUAAG | 12130 | CUUAUUUUCUGCAGGGGA |
| | | | | G |
| siRNA 5777 | 5777 | UCCCCUGCAGAAAAUAAGC | 12131 | GCUUAUUUUCUGCAGGGG |
| | | | | A |
| siRNA 5778 | 5778 | CCCCUGCAGAAAAUAAGCA | 12132 | UGCUUAUUUUCUGCAGGG |
| | | | | G |
| siRNA 5779 | 5779 | CCCUGCAGAAAAUAAGCAC | 12133 | GUGCUUAUUUUCUGCAGG |
| | | | | G |
| siRNA 5780 | 5780 | CCUGCAGAAAAUAAGCACA | 12134 | UGUGCUUAUUUUCUGCAG |
| | | | | G |
| siRNA 5781 | 5781 | CUGCAGAAAAUAAGCACAG | 12135 | CUGUGCUUAUUUUCUGCAG |
| siRNA 5782 | 5782 | UGCAGAAAAUAAGCACAG | 12136 | UCUGUGCUUAUUUUCUGCA |
| | | A | | |
| siRNA 5783 | 5783 | GCAGAAAAUAAGCACAGA | 12137 | UUCUGUGCUUAUUUUCUGC |
| | | A | | |
| siRNA 5784 | 5784 | CAGAAAAUAAGCACAGAA | 12138 | UUUCUGUGCUUAUUUUCU |
| | | A | | G |
| siRNA 5785 | 5785 | AGAAAAUAAGCACAGAAA | 12139 | UUUUCUGUGCUUAUUUUC |
| | | A | | U |
| siRNA 5786 | 5786 | GAAAAUAAGCACAGAAAA | 12140 | AUUUUCUGUGCUUAUUUU |
| | | U | | C |
| siRNA 5787 | 5787 | AAAAUAAGCACAGAAAAU | 12141 | AAUUUUCUGUGCUUAUUU |
| | | U | | U |
| siRNA 5788 | 5788 | AAAUAAGCACAGAAAAUU | 12142 | UAAUUUUCUGUGCUUAUU |
| | | A | | U |
| siRNA 5789 | 5789 | AAUAAGCACAGAAAAUUA | 12143 | AUAAUUUUCUGUGCUUAU |
| | | U | | U |
| siRNA 5790 | 5790 | AUAAGCACAGAAAAUUAU | 12144 | UAUAAUUUUCUGUGCUUA |
| | | A | | U |
| siRNA 5791 | 5791 | UAAGCACAGAAAAUUAUA | 12145 | UUAUAAUUUUCUGUGCUU |
| | | A | | A |
| siRNA 5792 | 5792 | AAGCACAGAAAAUUAUAA | 12146 | AUUAUAAUUUUCUGUGCU |
| | | U | | U |
| siRNA 5793 | 5793 | AGCACAGAAAAUUAUAAU | 12147 | CAUUAUAAUUUUCUGUGC |
| | | G | | U |
| siRNA 5794 | 5794 | GCACAGAAAAUUAUAAUG | 12148 | ACAUUAUAAUUUUCUGUG |
| | | U | | C |
| siRNA 5795 | 5795 | CACAGAAAAUUAUAAUGU | 12149 | UACAUUAUAAUUUUCUGU |
| | | A | | G |
| siRNA 5796 | 5796 | ACAGAAAAUUAUAAUGUA | 12150 | CUACAUUAUAAUUUUCUG |
| | | G | | U |
| siRNA 5797 | 5797 | CAGAAAAUUAUAAUGUAG | 12151 | CCUACAUUAUAAUUUUCUG |
| | | G | | |
| siRNA 5798 | 5798 | AGAAAAUUAUAAUGUAGG | 12152 | ACCUACAUUAUAAUUUUCU |
| | | U | | |
| siRNA 5799 | 5799 | GAAAAUUAUAAUGUAGGU | 12153 | CACCUACAUUAUAAUUUUC |
| | | G | | |
| siRNA 5800 | 5800 | AAAAUUAUAAUGUAGGUG | 12154 | UCACCUACAUUAUAAUUUU |
| | | A | | |
| siRNA 5801 | 5801 | AAAUUAUAAUGUAGGUGA | 12155 | AUCACCUACAUUAUAAUUU |
| | | U | | |
| siRNA 5802 | 5802 | AAUUAUAAUGUAGGUGAU | 12156 | GAUCACCUACAUUAUAAUU |
| | | C | | |
| siRNA 5803 | 5803 | AUUAUAAUGUAGGUGAUC | 12157 | CGAUCACCUACAUUAUAAU |
| | | G | | |
| siRNA 5804 | 5804 | UUAUAAUGUAGGUGAUCG | 12158 | CCGAUCACCUACAUUAUAA |
| | | G | | |
| siRNA 5805 | 5805 | UAUAAUGUAGGUGAUCGG | 12159 | UCCGAUCACCUACAUUAUA |
| | | A | | |
| siRNA 5806 | 5806 | AUAAUGUAGGUGAUCGGA | 12160 | CUCCGAUCACCUACAUUAU |
| | | G | | |
| siRNA 5807 | 5807 | UAAUGUAGGUGAUCGGAG | 12161 | GCUCCGAUCACCUACAUUA |
| | | C | | |
| siRNA 5808 | 5808 | AAUGUAGGUGAUCGGAGC | 12162 | AGCUCCGAUCACCUACAUU |
| | | U | | |
| siRNA 5809 | 5809 | AUGUAGGUGAUCGGAGCU | 12163 | GAGCUCCGAUCACCUACAU |
| | | C | | |
| siRNA 5810 | 5810 | UGUAGGUGAUCGGAGCUC | 12164 | AGAGCUCCGAUCACCUACA |
| | | U | | |
| siRNA 5811 | 5811 | GUAGGUGAUCGGAGCUCU | 12165 | AAGAGCUCCGAUCACCUAC |
| | | U | | |
| siRNA 5812 | 5812 | UAGGUGAUCGGAGCUCUU | 12166 | AAAGAGCUCCGAUCACCUA |
| | | U | | |
| siRNA 5813 | 5813 | AGGUGAUCGGAGCUCUUUC | 12167 | GAAAGAGCUCCGAUCACCU |
| siRNA 5814 | 5814 | GGUGAUCGGAGCUCUUUCC | 12168 | GGAAAGAGCUCCGAUCACC |
| siRNA 5815 | 5815 | GUGAUCGGAGCUCUUUCCU | 12169 | AGGAAAGAGCUCCGAUCAC |
| siRNA 5816 | 5816 | UGAUCGGAGCUCUUUCCUU | 12170 | AAGGAAAGAGCUCCGAUCA |
| siRNA 5817 | 5817 | GAUCGGAGCUCUUUCCUUU | 12171 | AAAGGAAAGAGCUCCGAUC |
| siRNA 5818 | 5818 | AUCGGAGCUCUUUCCUUUG | 12172 | CAAAGGAAAGAGCUCCGAU |
| siRNA 5819 | 5819 | UCGGAGCUCUUUCCUUUGA | 12173 | UCAAAGGAAAGAGCUCCGA |
| siRNA 5820 | 5820 | CGGAGCUCUUUCCUUUGAU | 12174 | AUCAAAGGAAAGAGCUCCG |
| siRNA 5821 | 5821 | GGAGCUCUUUCCUUUGAUA | 12175 | UAUCAAAGGAAAGAGCUCC |
| siRNA 5822 | 5822 | GAGCUCUUUCCUUUGAUAG | 12176 | CUAUCAAAGGAAAGAGCUC |
| siRNA 5823 | 5823 | AGCUCUUUCCUUUGAUAGA | 12177 | UCUAUCAAAGGAAAGAGC |
| | | | | U |
| siRNA 5824 | 5824 | GCUCUUUCCUUUGAUAGAG | 12178 | CUCUAUCAAAGGAAAGAGC |
| siRNA 5825 | 5825 | CUCUUUCCUUUGAUAGAGA | 12179 | UCUCUAUCAAAGGAAAGA |
| | | | | G |
| siRNA 5826 | 5826 | UCUUUCCUUUGAUAGAGA | 12180 | CUCUCUAUCAAAGGAAAGA |
| | | G | | |
| siRNA 5827 | 5827 | CUUUCCUUUGAUAGAGAG | 12181 | UCUCUCUAUCAAAGGAAAG |
| | | A | | |
| siRNA 5828 | 5828 | UUUCCUUUGAUAGAGAGA | 12182 | UUCUCUCUAUCAAAGGAAA |
| | | A | | |
| siRNA 5829 | 5829 | UUCCUUUGAUAGAGAGAA | 12183 | GUUCUCUCUAUCAAAGGAA |
| | | C | | |
| siRNA 5830 | 5830 | UCCUUUGAUAGAGAGAAC | 12184 | UGUUCUCUCUAUCAAAGGA |
| | | A | | |
| siRNA 5831 | 5831 | CCUUUGAUAGAGAGAACA | 12185 | CUGUUCUCUCUAUCAAAGG |
| | | G | | |
| siRNA 5832 | 5832 | CUUUGAUAGAGAGAACAG | 12186 | GCUGUUCUCUCUAUCAAAG |
| | | C | | |
| siRNA 5833 | 5833 | UUUGAUAGAGAGAACAGC | 12187 | GGCUGUUCUCUCUAUCAAA |
| | | C | | |
| siRNA 5834 | 5834 | UUGAUAGAGAGAACAGCCC | 12188 | GGGCUGUUCUCUCUAUCAA |
| siRNA 5835 | 5835 | UGAUAGAGAGAACAGCCCC | 12189 | GGGGCUGUUCUCUCUAUCA |
| siRNA 5836 | 5836 | GAUAGAGAGAACAGCCCCA | 12190 | UGGGGCUGUUCUCUCUAUC |
| siRNA 5837 | 5837 | AUAGAGAGAACAGCCCCAA | 12191 | UUGGGGCUGUUCUCUCUAU |
| siRNA 5838 | 5838 | UAGAGAGAACAGCCCCAAU | 12192 | AUUGGGGCUGUUCUCUCUA |
| siRNA 5839 | 5839 | AGAGAGAACAGCCCCAAUG | 12193 | CAUUGGGGCUGUUCUCUCU |
| siRNA 5840 | 5840 | GAGAGAACAGCCCCAAUGA | 12194 | UCAUUGGGGCUGUUCUCUC |
| siRNA 5841 | 5841 | AGAGAACAGCCCCAAUGAU | 12195 | AUCAUUGGGGCUGUUCUCU |
| siRNA 5842 | 5842 | GAGAACAGCCCCAAUGAUC | 12196 | GAUCAUUGGGGCUGUUCUC |
| siRNA 5843 | 5843 | AGAACAGCCCCAAUGAUCC | 12197 | GGAUCAUUGGGGCUGUUC |
| | | | | U |
| siRNA 5844 | 5844 | GAACAGCCCCAAUGAUCCU | 12198 | AGGAUCAUUGGGGCUGUU |
| | | | | C |
| siRNA 5845 | 5845 | AACAGCCCCAAUGAUCCUG | 12199 | CAGGAUCAUUGGGGCUGU |
| | | | | U |
| siRNA 5846 | 5846 | ACAGCCCCAAUGAUCCUGG | 12200 | CCAGGAUCAUUGGGGCUGU |
| siRNA 5847 | 5847 | CAGCCCCAAUGAUCCUGGC | 12201 | GCCAGGAUCAUUGGGGCUG |
| siRNA 5848 | 5848 | AGCCCCAAUGAUCCUGGCU | 12202 | AGCCAGGAUCAUUGGGGCU |
| siRNA 5849 | 5849 | GCCCCAAUGAUCCUGGCUU | 12203 | AAGCCAGGAUCAUUGGGGC |
| siRNA 5850 | 5850 | CCCCAAUGAUCCUGGCUUU | 12204 | AAAGCCAGGAUCAUUGGG |
| | | | | G |
| siRNA 5851 | 5851 | CCCAAUGAUCCUGGCUUUU | 12205 | AAAAGCCAGGAUCAUUGG |
| | | | | G |
| siRNA 5852 | 5852 | CCAAUGAUCCUGGCUUUUU | 12206 | AAAAAGCCAGGAUCAUUG |
| | | | | G |
| siRNA 5853 | 5853 | CAAUGAUCCUGGCUUUUUC | 12207 | GAAAAAGCCAGGAUCAUU |
| | | | | G |
| siRNA 5854 | 5854 | AAUGAUCCUGGCUUUUUCA | 12208 | UGAAAAAGCCAGGAUCAU |
| | | | | U |
| siRNA 5855 | 5855 | AUGAUCCUGGCUUUUUCAC | 12209 | GUGAAAAAGCCAGGAUCA |
| | | | | U |
| siRNA 5856 | 5856 | UGAUCCUGGCUUUUUCACU | 12210 | AGUGAAAAAGCCAGGAUC |
| | | | | A |
| siRNA 5857 | 5857 | GAUCCUGGCUUUUUCACUG | 12211 | CAGUGAAAAAGCCAGGAUC |
| siRNA 5858 | 5858 | AUCCUGGCUUUUUCACUGA | 12212 | UCAGUGAAAAAGCCAGGA |
| | | | | U |
| siRNA 5859 | 5859 | UCCUGGCUUUUUCACUGAA | 12213 | UUCAGUGAAAAAGCCAGG |
| | | | | A |
| siRNA 5860 | 5860 | CCUGGCUUUUUCACUGAAC | 12214 | GUUCAGUGAAAAAGCCAG |
| | | | | G |
| siRNA 5861 | 5861 | CUGGCUUUUUCACUGAACG | 12215 | CGUUCAGUGAAAAAGCCAG |
| siRNA 5862 | 5862 | UGGCUUUUUCACUGAACGU | 12216 | ACGUUCAGUGAAAAAGCCA |
| siRNA 5863 | 5863 | GGCUUUUUCACUGAACGUA | 12217 | UACGUUCAGUGAAAAAGCC |
| siRNA 5864 | 5864 | GCUUUUUCACUGAACGUAU | 12218 | AUACGUUCAGUGAAAAAG |
| | | | | C |
| siRNA 5865 | 5865 | CUUUUUCACUGAACGUAUC | 12219 | GAUACGUUCAGUGAAAAA |
| | | | | G |
| siRNA 5866 | 5866 | UUUUUCACUGAACGUAUCA | 12220 | UGAUACGUUCAGUGAAAA |
| | | | | A |
| siRNA 5867 | 5867 | UUUUCACUGAACGUAUCAG | 12221 | CUGAUACGUUCAGUGAAA |
| | | | | A |
| siRNA 5868 | 5868 | UUUCACUGAACGUAUCAGA | 12222 | UCUGAUACGUUCAGUGAA |
| | | | | A |
| siRNA 5869 | 5869 | UUCACUGAACGUAUCAGAA | 12223 | UUCUGAUACGUUCAGUGA |
| | | | | A |
| siRNA 5870 | 5870 | UCACUGAACGUAUCAGAAU | 12224 | AUUCUGAUACGUUCAGUG |
| | | | | A |
| siRNA 5871 | 5871 | CACUGAACGUAUCAGAAUA | 12225 | UAUUCUGAUACGUUCAGU |
| | | | | G |
| siRNA 5872 | 5872 | ACUGAACGUAUCAGAAUAC | 12226 | GUAUUCUGAUACGUUCAG |
| | | | | U |
| siRNA 5873 | 5873 | CUGAACGUAUCAGAAUACA | 12227 | UGUAUUCUGAUACGUUCA |
| | | | | G |
| siRNA 5874 | 5874 | UGAACGUAUCAGAAUACA | 12228 | AUGUAUUCUGAUACGUUC |
| | | U | | A |
| siRNA 5875 | 5875 | GAACGUAUCAGAAUACAU | 12229 | CAUGUAUUCUGAUACGUUC |
| | | G | | |
| siRNA 5876 | 5876 | AACGUAUCAGAAUACAUG | 12230 | CCAUGUAUUCUGAUACGUU |
| | | G | | |
| siRNA 5877 | 5877 | ACGUAUCAGAAUACAUGG | 12231 | UCCAUGUAUUCUGAUACGU |
| | | A | | |
| siRNA 5878 | 5878 | CGUAUCAGAAUACAUGGA | 12232 | AUCCAUGUAUUCUGAUACG |
| | | U | | |
| siRNA 5879 | 5879 | GUAUCAGAAUACAUGGAU | 12233 | CAUCCAUGUAUUCUGAUAC |
| | | G | | |
| siRNA 5880 | 5880 | UAUCAGAAUACAUGGAUG | 12234 | UCAUCCAUGUAUUCUGAUA |
| | | A | | |
| siRNA 5881 | 5881 | AUCAGAAUACAUGGAUGA | 12235 | UUCAUCCAUGUAUUCUGAU |
| | | A | | |
| siRNA 5882 | 5882 | UCAGAAUACAUGGAUGAA | 12236 | AUUCAUCCAUGUAUUCUGA |
| | | U | | |
| siRNA 5883 | 5883 | CAGAAUACAUGGAUGAAU | 12237 | AAUUCAUCCAUGUAUUCUG |
| | | U | | |
| siRNA 5884 | 5884 | AGAAUACAUGGAUGAAUU | 12238 | CAAUUCAUCCAUGUAUUCU |
| | | G | | |
| siRNA 5885 | 5885 | GAAUACAUGGAUGAAUUG | 12239 | CCAAUUCAUCCAUGUAUUC |
| | | G | | |
| siRNA 5886 | 5886 | AAUACAUGGAUGAAUUGG | 12240 | CCCAAUUCAUCCAUGUAUU |
| | | G | | |
| siRNA 5887 | 5887 | AUACAUGGAUGAAUUGGG | 12241 | CCCCAAUUCAUCCAUGUAU |
| | | G | | |
| siRNA 5888 | 5888 | UACAUGGAUGAAUUGGGG | 12242 | ACCCCAAUUCAUCCAUGUA |
| | | U | | |
| siRNA 5889 | 5889 | ACAUGGAUGAAUUGGGGU | 12243 | UACCCCAAUUCAUCCAUGU |
| | | A | | |
| siRNA 5890 | 5890 | CAUGGAUGAAUUGGGGUA | 12244 | UUACCCCAAUUCAUCCAUG |
| | | A | | |
| siRNA 5891 | 5891 | AUGGAUGAAUUGGGGUAA | 12245 | UUUACCCCAAUUCAUCCAU |
| | | A | | |
| siRNA 5892 | 5892 | UGGAUGAAUUGGGGUAAA | 12246 | AUUUACCCCAAUUCAUCCA |
| | | U | | |
| siRNA 5893 | 5893 | GGAUGAAUUGGGGUAAAU | 12247 | UAUUUACCCCAAUUCAUCC |
| | | A | | |
| siRNA 5894 | 5894 | GAUGAAUUGGGGUAAAUA | 12248 | UUAUUUACCCCAAUUCAUC |
| | | A | | |
| siRNA 5895 | 5895 | AUGAAUUGGGGUAAAUAA | 12249 | CUUAUUUACCCCAAUUCAU |
| | | G | | |
| siRNA 5896 | 5896 | UGAAUUGGGGUAAAUAAG | 12250 | CCUUAUUUACCCCAAUUCA |
| | | G | | |
| siRNA 5897 | 5897 | GAAUUGGGGUAAAUAAGG | 12251 | ACCUUAUUUACCCCAAUUC |
| | | U | | |
| siRNA 5898 | 5898 | AAUUGGGGUAAAUAAGGU | 12252 | AACCUUAUUUACCCCAAUU |
| | | U | | |
| siRNA 5899 | 5899 | AUUGGGGUAAAUAAGGUU | 12253 | AAACCUUAUUUACCCCAAU |
| | | U | | |
| siRNA 5900 | 5900 | UUGGGGUAAAUAAGGUUU | 12254 | AAAACCUUAUUUACCCCAA |
| | | U | | |
| siRNA 5901 | 5901 | UGGGGUAAAUAAGGUUUU | 12255 | UAAAACCUUAUUUACCCCA |
| | | A | | |
| siRNA 5902 | 5902 | GGGGUAAAUAAGGUUUUA | 12256 | UUAAAACCUUAUUUACCCC |
| | | A | | |
| siRNA 5903 | 5903 | GGGUAAAUAAGGUUUUAA | 12257 | AUUAAAACCUUAUUUACCC |
| | | U | | |
| siRNA 5904 | 5904 | GGUAAAUAAGGUUUUAAU | 12258 | AAUUAAAACCUUAUUUACC |
| | | U | | |
| siRNA 5905 | 5905 | GUAAAUAAGGUUUUAAUU | 12259 | GAAUUAAAACCUUAUUUA |
| | | C | | C |
| siRNA 5906 | 5906 | UAAAUAAGGUUUUAAUUC | 12260 | UGAAUUAAAACCUUAUUU |
| | | A | | A |
| siRNA 5907 | 5907 | AAAUAAGGUUUUAAUUCA | 12261 | CUGAAUUAAAACCUUAUU |
| | | G | | U |
| siRNA 5908 | 5908 | AAUAAGGUUUUAAUUCAG | 12262 | UCUGAAUUAAAACCUUAU |
| | | A | | U |
| siRNA 5909 | 5909 | AUAAGGUUUUAAUUCAGA | 12263 | AUCUGAAUUAAAACCUUA |
| | | U | | U |
| siRNA 5910 | 5910 | UAAGGUUUUAAUUCAGAU | 12264 | GAUCUGAAUUAAAACCUU |
| | | C | | A |
| siRNA 5911 | 5911 | AAGGUUUUAAUUCAGAUC | 12265 | AGAUCUGAAUUAAAACCU |
| | | U | | U |
| siRNA 5912 | 5912 | AGGUUUUAAUUCAGAUCU | 12266 | UAGAUCUGAAUUAAAACC |
| | | A | | U |
| siRNA 5913 | 5913 | GGUUUUAAUUCAGAUCUA | 12267 | CUAGAUCUGAAUUAAAACC |
| | | G | | |
| siRNA 5914 | 5914 | GUUUUAAUUCAGAUCUAG | 12268 | UCUAGAUCUGAAUUAAAA |
| | | A | | C |
| siRNA 5915 | 5915 | UUUUAAUUCAGAUCUAGA | 12269 | UUCUAGAUCUGAAUUAAA |
| | | A | | A |
| siRNA 5916 | 5916 | UUUAAUUCAGAUCUAGAA | 12270 | CUUCUAGAUCUGAAUUAA |
| | | G | | A |
| siRNA 5917 | 5917 | UUAAUUCAGAUCUAGAAG | 12271 | UCUUCUAGAUCUGAAUUA |
| | | A | | A |
| siRNA 5918 | 5918 | UAAUUCAGAUCUAGAAGA | 12272 | UUCUUCUAGAUCUGAAUU |
| | | A | | A |
| siRNA 5919 | 5919 | AAUUCAGAUCUAGAAGAA | 12273 | UUUCUUCUAGAUCUGAAU |
| | | A | | U |
| siRNA 5920 | 5920 | AUUCAGAUCUAGAAGAAA | 12274 | CUUUCUUCUAGAUCUGAAU |
| | | G | | |
| siRNA 5921 | 5921 | UUCAGAUCUAGAAGAAAG | 12275 | ACUUUCUUCUAGAUCUGAA |
| | | U | | |
| siRNA 5922 | 5922 | UCAGAUCUAGAAGAAAGU | 12276 | UACUUUCUUCUAGAUCUGA |
| | | A | | |
| siRNA 5923 | 5923 | CAGAUCUAGAAGAAAGUA | 12277 | AUACUUUCUUCUAGAUCUG |
| | | U | | |
| siRNA 5924 | 5924 | AGAUCUAGAAGAAAGUAU | 12278 | AAUACUUUCUUCUAGAUCU |
| | | U | | |
| siRNA 5925 | 5925 | GAUCUAGAAGAAAGUAUU | 12279 | CAAUACUUUCUUCUAGAUC |
| | | G | | |
| siRNA 5926 | 5926 | AUCUAGAAGAAAGUAUUG | 12280 | ACAAUACUUUCUUCUAGAU |
| | | U | | |
| siRNA 5927 | 5927 | UCUAGAAGAAAGUAUUGU | 12281 | UACAAUACUUUCUUCUAGA |
| | | A | | |
| siRNA 5928 | 5928 | CUAGAAGAAAGUAUUGUA | 12282 | GUACAAUACUUUCUUCUAG |
| | | C | | |
| siRNA 5929 | 5929 | UAGAAGAAAGUAUUGUAC | 12283 | CGUACAAUACUUUCUUCUA |
| | | G | | |
| siRNA 5930 | 5930 | AGAAGAAAGUAUUGUACG | 12284 | ACGUACAAUACUUUCUUCU |
| | | U | | |
| siRNA 5931 | 5931 | GAAGAAAGUAUUGUACGU | 12285 | AACGUACAAUACUUUCUUC |
| | | U | | |
| siRNA 5932 | 5932 | AAGAAAGUAUUGUACGUU | 12286 | AAACGUACAAUACUUUCUU |
| | | U | | |
| siRNA 5933 | 5933 | AGAAAGUAUUGUACGUUU | 12287 | CAAACGUACAAUACUUUCU |
| | | G | | |
| siRNA 5934 | 5934 | GAAAGUAUUGUACGUUUG | 12288 | UCAAACGUACAAUACUUUC |
| | | A | | |
| siRNA 5935 | 5935 | AAAGUAUUGUACGUUUGA | 12289 | UUCAAACGUACAAUACUUU |
| | | A | | |
| siRNA 5936 | 5936 | AAGUAUUGUACGUUUGAA | 12290 | AUUCAAACGUACAAUACUU |
| | | U | | |
| siRNA 5937 | 5937 | AGUAUUGUACGUUUGAAU | 12291 | CAUUCAAACGUACAAUACU |
| | | G | | |
| siRNA 5938 | 5938 | GUAUUGUACGUUUGAAUG | 12292 | GCAUUCAAACGUACAAUAC |
| | | C | | |
| siRNA 5939 | 5939 | UAUUGUACGUUUGAAUGC | 12293 | UGCAUUCAAACGUACAAUA |
| | | A | | |
| siRNA 5940 | 5940 | AUUGUACGUUUGAAUGCA | 12294 | CUGCAUUCAAACGUACAAU |
| | | G | | |
| siRNA 5941 | 5941 | UUGUACGUUUGAAUGCAG | 12295 | UCUGCAUUCAAACGUACAA |
| | | A | | |
| siRNA 5942 | 5942 | UGUACGUUUGAAUGCAGA | 12296 | AUCUGCAUUCAAACGUACA |
| | | U | | |
| siRNA 5943 | 5943 | GUACGUUUGAAUGCAGAU | 12297 | AAUCUGCAUUCAAACGUAC |
| | | U | | |
| siRNA 5944 | 5944 | UACGUUUGAAUGCAGAUU | 12298 | AAAUCUGCAUUCAAACGUA |
| | | U | | |
| siRNA 5945 | 5945 | ACGUUUGAAUGCAGAUUU | 12299 | AAAAUCUGCAUUCAAACGU |
| | | U | | |
| siRNA 5946 | 5946 | CGUUUGAAUGCAGAUUUU | 12300 | AAAAAUCUGCAUUCAAACG |
| | | U | | |
| siRNA 5947 | 5947 | GUUUGAAUGCAGAUUUUU | 12301 | UAAAAAUCUGCAUUCAAAC |
| | | A | | |
| siRNA 5948 | 5948 | UUUGAAUGCAGAUUUUUA | 12302 | AUAAAAAUCUGCAUUCAA |
| | | U | | A |
| siRNA 5949 | 5949 | UUGAAUGCAGAUUUUUAU | 12303 | GAUAAAAAUCUGCAUUCA |
| | | C | | A |
| siRNA 5950 | 5950 | UGAAUGCAGAUUUUUAUC | 12304 | GGAUAAAAAUCUGCAUUC |
| | | C | | A |
| siRNA 5951 | 5951 | GAAUGCAGAUUUUUAUCC | 12305 | UGGAUAAAAAUCUGCAUU |
| | | A | | C |
| siRNA 5952 | 5952 | AAUGCAGAUUUUUAUCCAC | 12306 | GUGGAUAAAAAUCUGCAU |
| | | | | U |
| siRNA 5953 | 5953 | AUGCAGAUUUUUAUCCACA | 12307 | UGUGGAUAAAAAUCUGCA |
| | | | | U |
| siRNA 5954 | 5954 | UGCAGAUUUUUAUCCACAG | 12308 | CUGUGGAUAAAAAUCUGC |
| | | | | A |
| siRNA 5955 | 5955 | GCAGAUUUUUAUCCACAGA | 12309 | UCUGUGGAUAAAAAUCUG |
| | | | | C |
| siRNA 5956 | 5956 | CAGAUUUUUAUCCACAGAU | 12310 | AUCUGUGGAUAAAAAUCU |
| | | | | G |
| siRNA 5957 | 5957 | AGAUUUUUAUCCACAGAU | 12311 | UAUCUGUGGAUAAAAAUC |
| | | A | | U |
| siRNA 5958 | 5958 | GAUUUUUAUCCACAGAUA | 12312 | CUAUCUGUGGAUAAAAAU |
| | | G | | C |
| siRNA 5959 | 5959 | AUUUUUAUCCACAGAUAG | 12313 | ACUAUCUGUGGAUAAAAA |
| | | U | | U |
| siRNA 5960 | 5960 | UUUUUAUCCACAGAUAGU | 12314 | AACUAUCUGUGGAUAAAA |
| | | U | | A |
| siRNA 5961 | 5961 | UUUUAUCCACAGAUAGUU | 12315 | CAACUAUCUGUGGAUAAA |
| | | G | | A |
| siRNA 5962 | 5962 | UUUAUCCACAGAUAGUUG | 12316 | ACAACUAUCUGUGGAUAA |
| | | U | | A |
| siRNA 5963 | 5963 | UUAUCCACAGAUAGUUGU | 12317 | UACAACUAUCUGUGGAUA |
| | | A | | A |
| siRNA 5964 | 5964 | UAUCCACAGAUAGUUGUA | 12318 | CUACAACUAUCUGUGGAUA |
| | | G | | |
| siRNA 5965 | 5965 | AUCCACAGAUAGUUGUAG | 12319 | ACUACAACUAUCUGUGGAU |
| | | U | | |
| siRNA 5966 | 5966 | UCCACAGAUAGUUGUAGU | 12320 | CACUACAACUAUCUGUGGA |
| | | G | | |
| siRNA 5967 | 5967 | CCACAGAUAGUUGUAGUG | 12321 | ACACUACAACUAUCUGUGG |
| | | U | | |
| siRNA 5968 | 5968 | CACAGAUAGUUGUAGUGU | 12322 | AACACUACAACUAUCUGUG |
| | | U | | |
| siRNA 5969 | 5969 | ACAGAUAGUUGUAGUGUU | 12323 | AAACACUACAACUAUCUGU |
| | | U | | |
| siRNA 5970 | 5970 | CAGAUAGUUGUAGUGUUU | 12324 | UAAACACUACAACUAUCUG |
| | | A | | |
| siRNA 5971 | 5971 | AGAUAGUUGUAGUGUUUA | 12325 | CUAAACACUACAACUAUCU |
| | | G | | |
| siRNA 5972 | 5972 | GAUAGUUGUAGUGUUUAG | 12326 | UCUAAACACUACAACUAUC |
| | | A | | |
| siRNA 5973 | 5973 | AUAGUUGUAGUGUUUAGA | 12327 | GUCUAAACACUACAACUAU |
| | | C | | |
| siRNA 5974 | 5974 | UAGUUGUAGUGUUUAGAC | 12328 | UGUCUAAACACUACAACUA |
| | | A | | |
| siRNA 5975 | 5975 | AGUUGUAGUGUUUAGACA | 12329 | AUGUCUAAACACUACAACU |
| | | U | | |
| siRNA 5976 | 5976 | GUUGUAGUGUUUAGACAU | 12330 | CAUGUCUAAACACUACAAC |
| | | G | | |
| siRNA 5977 | 5977 | UUGUAGUGUUUAGACAUG | 12331 | UCAUGUCUAAACACUACAA |
| | | A | | |
| siRNA 5978 | 5978 | UGUAGUGUUUAGACAUGA | 12332 | GUCAUGUCUAAACACUACA |
| | | C | | |
| siRNA 5979 | 5979 | GUAGUGUUUAGACAUGAC | 12333 | UGUCAUGUCUAAACACUAC |
| | | A | | |
| siRNA 5980 | 5980 | UAGUGUUUAGACAUGACA | 12334 | CUGUCAUGUCUAAACACUA |
| | | G | | |
| siRNA 5981 | 5981 | AGUGUUUAGACAUGACAG | 12335 | CCUGUCAUGUCUAAACACU |
| | | G | | |
| siRNA 5982 | 5982 | GUGUUUAGACAUGACAGG | 12336 | UCCUGUCAUGUCUAAACAC |
| | | A | | |
| siRNA 5983 | 5983 | UGUUUAGACAUGACAGGA | 12337 | GUCCUGUCAUGUCUAAACA |
| | | C | | |
| siRNA 5984 | 5984 | GUUUAGACAUGACAGGACC | 12338 | GGUCCUGUCAUGUCUAAAC |
| siRNA 5985 | 5985 | UUUAGACAUGACAGGACCU | 12339 | AGGUCCUGUCAUGUCUAAA |
| siRNA 5986 | 5986 | UUAGACAUGACAGGACCUA | 12340 | UAGGUCCUGUCAUGUCUAA |
| siRNA 5987 | 5987 | UAGACAUGACAGGACCUAU | 12341 | AUAGGUCCUGUCAUGUCUA |
| siRNA 5988 | 5988 | AGACAUGACAGGACCUAUC | 12342 | GAUAGGUCCUGUCAUGUCU |
| siRNA 5989 | 5989 | GACAUGACAGGACCUAUCG | 12343 | CGAUAGGUCCUGUCAUGUC |
| siRNA 5990 | 5990 | ACAUGACAGGACCUAUCGU | 12344 | ACGAUAGGUCCUGUCAUGU |
| siRNA 5991 | 5991 | CAUGACAGGACCUAUCGUU | 12345 | AACGAUAGGUCCUGUCAUG |
| siRNA 5992 | 5992 | AUGACAGGACCUAUCGUUG | 12346 | CAACGAUAGGUCCUGUCAU |
| siRNA 5993 | 5993 | UGACAGGACCUAUCGUUGA | 12347 | UCAACGAUAGGUCCUGUCA |
| siRNA 5994 | 5994 | GACAGGACCUAUCGUUGAG | 12348 | CUCAACGAUAGGUCCUGUC |
| siRNA 5995 | 5995 | ACAGGACCUAUCGUUGAGG | 12349 | CCUCAACGAUAGGUCCUGU |
| siRNA 5996 | 5996 | CAGGACCUAUCGUUGAGGU | 12350 | ACCUCAACGAUAGGUCCUG |
| siRNA 5997 | 5997 | AGGACCUAUCGUUGAGGU | 12351 | AACCUCAACGAUAGGUCCU |
| | | U | | |
| siRNA 5998 | 5998 | GGACCUAUCGUUGAGGUU | 12352 | AAACCUCAACGAUAGGUCC |
| | | U | | |
| siRNA 5999 | 5999 | GACCUAUCGUUGAGGUUUC | 12353 | GAAACCUCAACGAUAGGUC |
| siRNA 6000 | 6000 | ACCUAUCGUUGAGGUUUCU | 12354 | AGAAACCUCAACGAUAGGU |
| siRNA 6001 | 6001 | CCUAUCGUUGAGGUUUCUA | 12355 | UAGAAACCUCAACGAUAGG |
| siRNA 6002 | 6002 | CUAUCGUUGAGGUUUCUA | 12356 | UUAGAAACCUCAACGAUAG |
| | | A | | |
| siRNA 6003 | 6003 | UAUCGUUGAGGUUUCUAA | 12357 | CUUAGAAACCUCAACGAUA |
| | | G | | |
| siRNA 6004 | 6004 | AUCGUUGAGGUUUCUAAG | 12358 | UCUUAGAAACCUCAACGAU |
| | | A | | |
| siRNA 6005 | 6005 | UCGUUGAGGUUUCUAAGA | 12359 | GUCUUAGAAACCUCAACGA |
| | | C | | |
| siRNA 6006 | 6006 | CGUUGAGGUUUCUAAGAC | 12360 | AGUCUUAGAAACCUCAACG |
| | | U | | |
| siRNA 6007 | 6007 | GUUGAGGUUUCUAAGACU | 12361 | AAGUCUUAGAAACCUCAAC |
| | | U | | |
| siRNA 6008 | 6008 | UUGAGGUUUCUAAGACUU | 12362 | UAAGUCUUAGAAACCUCAA |
| | | A | | |
| siRNA 6009 | 6009 | UGAGGUUUCUAAGACUUA | 12363 | GUAAGUCUUAGAAACCUCA |
| | | C | | |
| siRNA 6010 | 6010 | GAGGUUUCUAAGACUUAC | 12364 | AGUAAGUCUUAGAAACCUC |
| | | U | | |
| siRNA 6011 | 6011 | AGGUUUCUAAGACUUACU | 12365 | UAGUAAGUCUUAGAAACC |
| | | A | | U |
| siRNA 6012 | 6012 | GGUUUCUAAGACUUACUA | 12366 | AUAGUAAGUCUUAGAAAC |
| | | U | | C |
| siRNA 6013 | 6013 | GUUUCUAAGACUUACUAU | 12367 | CAUAGUAAGUCUUAGAAA |
| | | G | | C |
| siRNA 6014 | 6014 | UUUCUAAGACUUACUAUG | 12368 | CCAUAGUAAGUCUUAGAA |
| | | G | | A |
| siRNA 6015 | 6015 | UUCUAAGACUUACUAUGG | 12369 | CCCAUAGUAAGUCUUAGAA |
| | | G | | |
| siRNA 6016 | 6016 | UCUAAGACUUACUAUGGGC | 12370 | GCCCAUAGUAAGUCUUAGA |
| siRNA 6017 | 6017 | CUAAGACUUACUAUGGGCU | 12371 | AGCCCAUAGUAAGUCUUAG |
| siRNA 6018 | 6018 | UAAGACUUACUAUGGGCU | 12372 | CAGCCCAUAGUAAGUCUUA |
| | | G | | |
| siRNA 6019 | 6019 | AAGACUUACUAUGGGCUG | 12373 | ACAGCCCAUAGUAAGUCUU |
| | | U | | |
| siRNA 6020 | 6020 | AGACUUACUAUGGGCUGU | 12374 | UACAGCCCAUAGUAAGUCU |
| | | A | | |
| siRNA 6021 | 6021 | GACUUACUAUGGGCUGUA | 12375 | UUACAGCCCAUAGUAAGUC |
| | | A | | |
| siRNA 6022 | 6022 | ACUUACUAUGGGCUGUAA | 12376 | UUUACAGCCCAUAGUAAGU |
| | | A | | |
| siRNA 6023 | 6023 | CUUACUAUGGGCUGUAAAC | 12377 | GUUUACAGCCCAUAGUAAG |
| siRNA 6024 | 6024 | UUACUAUGGGCUGUAAACC | 12378 | GGUUUACAGCCCAUAGUAA |
| siRNA 6025 | 6025 | UACUAUGGGCUGUAAACCU | 12379 | AGGUUUACAGCCCAUAGUA |
| siRNA 6026 | 6026 | ACUAUGGGCUGUAAACCUG | 12380 | CAGGUUUACAGCCCAUAGU |
| siRNA 6027 | 6027 | CUAUGGGCUGUAAACCUGU | 12381 | ACAGGUUUACAGCCCAUAG |
| siRNA 6028 | 6028 | UAUGGGCUGUAAACCUGU | 12382 | AACAGGUUUACAGCCCAUA |
| | | U | | |
| siRNA 6029 | 6029 | AUGGGCUGUAAACCUGUU | 12383 | AAACAGGUUUACAGCCCAU |
| | | U | | |
| siRNA 6030 | 6030 | UGGGCUGUAAACCUGUUU | 12384 | AAAACAGGUUUACAGCCCA |
| | | U | | |
| siRNA 6031 | 6031 | GGGCUGUAAACCUGUUUU | 12385 | AAAAACAGGUUUACAGCCC |
| | | U | | |
| siRNA 6032 | 6032 | GGCUGUAAACCUGUUUUU | 12386 | AAAAAACAGGUUUACAGCC |
| | | U | | |
| siRNA 6033 | 6033 | GCUGUAAACCUGUUUUUU | 12387 | UAAAAAACAGGUUUACAG |
| | | A | | C |
| siRNA 6034 | 6034 | CUGUAAACCUGUUUUUUA | 12388 | UUAAAAAACAGGUUUACA |
| | | A | | G |
| siRNA 6035 | 6035 | UGUAAACCUGUUUUUUAA | 12389 | UUUAAAAAACAGGUUUAC |
| | | A | | A |
| siRNA 6036 | 6036 | GUAAACCUGUUUUUUAAA | 12390 | UUUUAAAAAACAGGUUUA |
| | | A | | C |
| siRNA 6037 | 6037 | UAAACCUGUUUUUUAAAA | 12391 | GUUUUAAAAAACAGGUUU |
| | | C | | A |
| siRNA 6038 | 6038 | AAACCUGUUUUUUAAAAC | 12392 | AGUUUUAAAAAACAGGUU |
| | | U | | U |
| siRNA 6039 | 6039 | AACCUGUUUUUUAAAACU | 12393 | UAGUUUUAAAAAACAGGU |
| | | A | | U |
| siRNA 6040 | 6040 | ACCUGUUUUUUAAAACUA | 12394 | AUAGUUUUAAAAAACAGG |
| | | U | | U |
| siRNA 6041 | 6041 | CCUGUUUUUUAAAACUAU | 12395 | AAUAGUUUUAAAAAACAG |
| | | U | | G |
| siRNA 6042 | 6042 | CUGUUUUUUAAAACUAUU | 12396 | AAAUAGUUUUAAAAAACA |
| | | U | | G |
| siRNA 6043 | 6043 | UGUUUUUUAAAACUAUUU | 12397 | AAAAUAGUUUUAAAAAAC |
| | | U | | A |
| siRNA 6044 | 6044 | GUUUUUUAAAACUAUUUU | 12398 | UAAAAUAGUUUUAAAAAA |
| | | A | | C |
| siRNA 6045 | 6045 | UUUUUUAAAACUAUUUUA | 12399 | CUAAAAUAGUUUUAAAAA |
| | | G | | A |
| siRNA 6046 | 6046 | UUUUUAAAACUAUUUUAG | 12400 | UCUAAAAUAGUUUUAAAA |
| | | A | | A |
| siRNA 6047 | 6047 | UUUUAAAACUAUUUUAGA | 12401 | UUCUAAAAUAGUUUUAAA |
| | | A | | A |
| siRNA 6048 | 6048 | UUUAAAACUAUUUUAGAA | 12402 | UUUCUAAAAUAGUUUUAA |
| | | A | | A |
| siRNA 6049 | 6049 | UUAAAACUAUUUUAGAAA | 12403 | GUUUCUAAAAUAGUUUUA |
| | | C | | A |
| siRNA 6050 | 6050 | UAAAACUAUUUUAGAAAC | 12404 | GGUUUCUAAAAUAGUUUU |
| | | C | | A |
| siRNA 6051 | 6051 | AAAACUAUUUUAGAAACC | 12405 | AGGUUUCUAAAAUAGUUU |
| | | U | | U |
| siRNA 6052 | 6052 | AAACUAUUUUAGAAACCU | 12406 | CAGGUUUCUAAAAUAGUU |
| | | G | | U |
| siRNA 6053 | 6053 | AACUAUUUUAGAAACCUG | 12407 | UCAGGUUUCUAAAAUAGU |
| | | A | | U |
| siRNA 6054 | 6054 | ACUAUUUUAGAAACCUGA | 12408 | CUCAGGUUUCUAAAAUAG |
| | | G | | U |
| siRNA 6055 | 6055 | CUAUUUUAGAAACCUGAG | 12409 | UCUCAGGUUUCUAAAAUA |
| | | A | | G |
| siRNA 6056 | 6056 | UAUUUUAGAAACCUGAGA | 12410 | GUCUCAGGUUUCUAAAAU |
| | | C | | A |
| siRNA 6057 | 6057 | AUUUUAGAAACCUGAGAC | 12411 | AGUCUCAGGUUUCUAAAA |
| | | U | | U |
| siRNA 6058 | 6058 | UUUUAGAAACCUGAGACU | 12412 | AAGUCUCAGGUUUCUAAA |
| | | U | | A |
| siRNA 6059 | 6059 | UUUAGAAACCUGAGACUU | 12413 | CAAGUCUCAGGUUUCUAAA |
| | | G | | |
| siRNA 6060 | 6060 | UUAGAAACCUGAGACUUGC | 12414 | GCAAGUCUCAGGUUUCUAA |
| siRNA 6061 | 6061 | UAGAAACCUGAGACUUGCC | 12415 | GGCAAGUCUCAGGUUUCUA |
| siRNA 6062 | 6062 | AGAAACCUGAGACUUGCCG | 12416 | CGGCAAGUCUCAGGUUUCU |
| siRNA 6063 | 6063 | GAAACCUGAGACUUGCCGU | 12417 | ACGGCAAGUCUCAGGUUUC |
| siRNA 6064 | 6064 | AAACCUGAGACUUGCCGUC | 12418 | GACGGCAAGUCUCAGGUUU |
| siRNA 6065 | 6065 | AACCUGAGACUUGCCGUCU | 12419 | AGACGGCAAGUCUCAGGUU |
| siRNA 6066 | 6066 | ACCUGAGACUUGCCGUCUG | 12420 | CAGACGGCAAGUCUCAGGU |
| siRNA 6067 | 6067 | CCUGAGACUUGCCGUCUGG | 12421 | CCAGACGGCAAGUCUCAGG |
| siRNA 6068 | 6068 | CUGAGACUUGCCGUCUGGC | 12422 | GCCAGACGGCAAGUCUCAG |
| siRNA 6069 | 6069 | UGAGACUUGCCGUCUGGCA | 12423 | UGCCAGACGGCAAGUCUCA |
| siRNA 6070 | 6070 | GAGACUUGCCGUCUGGCAU | 12424 | AUGCCAGACGGCAAGUCUC |
| siRNA 6071 | 6071 | AGACUUGCCGUCUGGCAUU | 12425 | AAUGCCAGACGGCAAGUCU |
| siRNA 6072 | 6072 | GACUUGCCGUCUGGCAUUU | 12426 | AAAUGCCAGACGGCAAGUC |
| siRNA 6073 | 6073 | ACUUGCCGUCUGGCAUUUU | 12427 | AAAAUGCCAGACGGCAAGU |
| siRNA 6074 | 6074 | CUUGCCGUCUGGCAUUUUA | 12428 | UAAAAUGCCAGACGGCAAG |
| siRNA 6075 | 6075 | UUGCCGUCUGGCAUUUUAG | 12429 | CUAAAAUGCCAGACGGCAA |
| siRNA 6076 | 6076 | UGCCGUCUGGCAUUUUAGU | 12430 | ACUAAAAUGCCAGACGGCA |
| siRNA 6077 | 6077 | GCCGUCUGGCAUUUUAGUU | 12431 | AACUAAAAUGCCAGACGGC |
| siRNA 6078 | 6078 | CCGUCUGGCAUUUUAGUUU | 12432 | AAACUAAAAUGCCAGACGG |
| siRNA 6079 | 6079 | CGUCUGGCAUUUUAGUUU | 12433 | UAAACUAAAAUGCCAGACG |
| | | A | | |
| siRNA 6080 | 6080 | GUCUGGCAUUUUAGUUUA | 12434 | UUAAACUAAAAUGCCAGAC |
| | | A | | |
| siRNA 6081 | 6081 | UCUGGCAUUUUAGUUUAA | 12435 | AUUAAACUAAAAUGCCAG |
| | | U | | A |
| siRNA 6082 | 6082 | CUGGCAUUUUAGUUUAAU | 12436 | UAUUAAACUAAAAUGCCA |
| | | A | | G |
| siRNA 6083 | 6083 | UGGCAUUUUAGUUUAAUA | 12437 | GUAUUAAACUAAAAUGCC |
| | | C | | A |
| siRNA 6084 | 6084 | GGCAUUUUAGUUUAAUAC | 12438 | UGUAUUAAACUAAAAUGC |
| | | A | | C |
| siRNA 6085 | 6085 | GCAUUUUAGUUUAAUACA | 12439 | UUGUAUUAAACUAAAAUG |
| | | A | | C |
| siRNA 6086 | 6086 | CAUUUUAGUUUAAUACAA | 12440 | UUUGUAUUAAACUAAAAU |
| | | A | | G |
| siRNA 6087 | 6087 | AUUUUAGUUUAAUACAAA | 12441 | GUUUGUAUUAAACUAAAA |
| | | C | | U |
| siRNA 6088 | 6088 | UUUUAGUUUAAUACAAAC | 12442 | AGUUUGUAUUAAACUAAA |
| | | U | | A |
| siRNA 6089 | 6089 | UUUAGUUUAAUACAAACU | 12443 | UAGUUUGUAUUAAACUAA |
| | | A | | A |
| siRNA 6090 | 6090 | UUAGUUUAAUACAAACUA | 12444 | UUAGUUUGUAUUAAACUA |
| | | A | | A |
| siRNA 6091 | 6091 | UAGUUUAAUACAAACUAA | 12445 | AUUAGUUUGUAUUAAACU |
| | | U | | A |
| siRNA 6092 | 6092 | AGUUUAAUACAAACUAAU | 12446 | CAUUAGUUUGUAUUAAAC |
| | | G | | U |
| siRNA 6093 | 6093 | GUUUAAUACAAACUAAUG | 12447 | UCAUUAGUUUGUAUUAAA |
| | | A | | C |
| siRNA 6094 | 6094 | UUUAAUACAAACUAAUGA | 12448 | AUCAUUAGUUUGUAUUAA |
| | | U | | A |
| siRNA 6095 | 6095 | UUAAUACAAACUAAUGAU | 12449 | AAUCAUUAGUUUGUAUUA |
| | | U | | A |
| siRNA 6096 | 6096 | UAAUACAAACUAAUGAUU | 12450 | CAAUCAUUAGUUUGUAUU |
| | | G | | A |
| siRNA 6097 | 6097 | AAUACAAACUAAUGAUUG | 12451 | GCAAUCAUUAGUUUGUAU |
| | | C | | U |
| siRNA 6098 | 6098 | AUACAAACUAAUGAUUGC | 12452 | UGCAAUCAUUAGUUUGUA |
| | | A | | U |
| siRNA 6099 | 6099 | UACAAACUAAUGAUUGCA | 12453 | AUGCAAUCAUUAGUUUGU |
| | | U | | A |
| siRNA 6100 | 6100 | ACAAACUAAUGAUUGCAU | 12454 | AAUGCAAUCAUUAGUUUG |
| | | U | | U |
| siRNA 6101 | 6101 | CAAACUAAUGAUUGCAUU | 12455 | AAAUGCAAUCAUUAGUUU |
| | | U | | G |
| siRNA 6102 | 6102 | AAACUAAUGAUUGCAUUU | 12456 | CAAAUGCAAUCAUUAGUU |
| | | G | | U |
| siRNA 6103 | 6103 | AACUAAUGAUUGCAUUUG | 12457 | UCAAAUGCAAUCAUUAGU |
| | | A | | U |
| siRNA 6104 | 6104 | ACUAAUGAUUGCAUUUGA | 12458 | UUCAAAUGCAAUCAUUAG |
| | | A | | U |
| siRNA 6105 | 6105 | CUAAUGAUUGCAUUUGAA | 12459 | UUUCAAAUGCAAUCAUUA |
| | | A | | G |
| siRNA 6106 | 6106 | UAAUGAUUGCAUUUGAAA | 12460 | CUUUCAAAUGCAAUCAUUA |
| | | G | | |
| siRNA 6107 | 6107 | AAUGAUUGCAUUUGAAAG | 12461 | UCUUUCAAAUGCAAUCAUU |
| | | A | | |
| siRNA 6108 | 6108 | AUGAUUGCAUUUGAAAGA | 12462 | CUCUUUCAAAUGCAAUCAU |
| | | G | | |
| siRNA 6109 | 6109 | UGAUUGCAUUUGAAAGAG | 12463 | UCUCUUUCAAAUGCAAUCA |
| | | A | | |
| siRNA 6110 | 6110 | GAUUGCAUUUGAAAGAGA | 12464 | AUCUCUUUCAAAUGCAAUC |
| | | U | | |
| siRNA 6111 | 6111 | AUUGCAUUUGAAAGAGAU | 12465 | AAUCUCUUUCAAAUGCAAU |
| | | U | | |
| siRNA 6112 | 6112 | UUGCAUUUGAAAGAGAUU | 12466 | GAAUCUCUUUCAAAUGCAA |
| | | C | | |
| siRNA 6113 | 6113 | UGCAUUUGAAAGAGAUUC | 12467 | AGAAUCUCUUUCAAAUGCA |
| | | U | | |
| siRNA 6114 | 6114 | GCAUUUGAAAGAGAUUCU | 12468 | AAGAAUCUCUUUCAAAUGC |
| | | U | | |
| siRNA 6115 | 6115 | CAUUUGAAAGAGAUUCUU | 12469 | CAAGAAUCUCUUUCAAAUG |
| | | G | | |
| siRNA 6116 | 6116 | AUUUGAAAGAGAUUCUUG | 12470 | UCAAGAAUCUCUUUCAAAU |
| | | A | | |
| siRNA 6117 | 6117 | UUUGAAAGAGAUUCUUGA | 12471 | GUCAAGAAUCUCUUUCAAA |
| | | C | | |
| siRNA 6118 | 6118 | UUGAAAGAGAUUCUUGAC | 12472 | GGUCAAGAAUCUCUUUCAA |
| | | C | | |
| siRNA 6119 | 6119 | UGAAAGAGAUUCUUGACC | 12473 | AGGUCAAGAAUCUCUUUCA |
| | | U | | |
| siRNA 6120 | 6120 | GAAAGAGAUUCUUGACCU | 12474 | AAGGUCAAGAAUCUCUUUC |
| | | U | | |
| siRNA 6121 | 6121 | AAAGAGAUUCUUGACCUU | 12475 | UAAGGUCAAGAAUCUCUU |
| | | A | | U |
| siRNA 6122 | 6122 | AAGAGAUUCUUGACCUUA | 12476 | AUAAGGUCAAGAAUCUCU |
| | | U | | U |
| siRNA 6123 | 6123 | AGAGAUUCUUGACCUUAU | 12477 | AAUAAGGUCAAGAAUCUC |
| | | U | | U |
| siRNA 6124 | 6124 | GAGAUUCUUGACCUUAUU | 12478 | AAAUAAGGUCAAGAAUCU |
| | | U | | C |
| siRNA 6125 | 6125 | AGAUUCUUGACCUUAUUUC | 12479 | GAAAUAAGGUCAAGAAUC |
| | | | | U |
| siRNA 6126 | 6126 | GAUUCUUGACCUUAUUUCU | 12480 | AGAAAUAAGGUCAAGAAU |
| | | | | C |
| siRNA 6127 | 6127 | AUUCUUGACCUUAUUUCUA | 12481 | UAGAAAUAAGGUCAAGAA |
| | | | | U |
| siRNA 6128 | 6128 | UUCUUGACCUUAUUUCUAA | 12482 | UUAGAAAUAAGGUCAAGA |
| | | | | A |
| siRNA 6129 | 6129 | UCUUGACCUUAUUUCUAAA | 12483 | UUUAGAAAUAAGGUCAAG |
| | | | | A |
| siRNA 6130 | 6130 | CUUGACCUUAUUUCUAAAC | 12484 | GUUUAGAAAUAAGGUCAA |
| | | | | G |
| siRNA 6131 | 6131 | UUGACCUUAUUUCUAAACG | 12485 | CGUUUAGAAAUAAGGUCA |
| | | | | A |
| siRNA 6132 | 6132 | UGACCUUAUUUCUAAACGU | 12486 | ACGUUUAGAAAUAAGGUC |
| | | | | A |
| siRNA 6133 | 6133 | GACCUUAUUUCUAAACGUC | 12487 | GACGUUUAGAAAUAAGGU |
| | | | | C |
| siRNA 6134 | 6134 | ACCUUAUUUCUAAACGUCU | 12488 | AGACGUUUAGAAAUAAGG |
| | | | | U |
| siRNA 6135 | 6135 | CCUUAUUUCUAAACGUCUA | 12489 | UAGACGUUUAGAAAUAAG |
| | | | | G |
| siRNA 6136 | 6136 | CUUAUUUCUAAACGUCUAG | 12490 | CUAGACGUUUAGAAAUAA |
| | | | | G |
| siRNA 6137 | 6137 | UUAUUUCUAAACGUCUAG | 12491 | UCUAGACGUUUAGAAAUA |
| | | A | | A |
| siRNA 6138 | 6138 | UAUUUCUAAACGUCUAGA | 12492 | CUCUAGACGUUUAGAAAU |
| | | G | | A |
| siRNA 6139 | 6139 | AUUUCUAAACGUCUAGAGC | 12493 | GCUCUAGACGUUUAGAAA |
| | | | | U |
| siRNA 6140 | 6140 | UUUCUAAACGUCUAGAGCU | 12494 | AGCUCUAGACGUUUAGAA |
| | | | | A |
| siRNA 6141 | 6141 | UUCUAAACGUCUAGAGCUC | 12495 | GAGCUCUAGACGUUUAGA |
| | | | | A |
| siRNA 6142 | 6142 | UCUAAACGUCUAGAGCUCU | 12496 | AGAGCUCUAGACGUUUAG |
| | | | | A |
| siRNA 6143 | 6143 | CUAAACGUCUAGAGCUCUG | 12497 | CAGAGCUCUAGACGUUUAG |
| siRNA 6144 | 6144 | UAAACGUCUAGAGCUCUGA | 12498 | UCAGAGCUCUAGACGUUUA |
| siRNA 6145 | 6145 | AAACGUCUAGAGCUCUGAA | 12499 | UUCAGAGCUCUAGACGUUU |
| siRNA 6146 | 6146 | AACGUCUAGAGCUCUGAAA | 12500 | UUUCAGAGCUCUAGACGUU |
| siRNA 6147 | 6147 | ACGUCUAGAGCUCUGAAAU | 12501 | AUUUCAGAGCUCUAGACGU |
| siRNA 6148 | 6148 | CGUCUAGAGCUCUGAAAUG | 12502 | CAUUUCAGAGCUCUAGACG |
| siRNA 6149 | 6149 | GUCUAGAGCUCUGAAAUG | 12503 | |
| | | U | | ACAUUUCAGAGCUCUAGAC |
| siRNA 6150 | 6150 | UCUAGAGCUCUGAAAUGUC | 12504 | GACAUUUCAGAGCUCUAGA |
| siRNA 6151 | 6151 | CUAGAGCUCUGAAAUGUCU | 12505 | AGACAUUUCAGAGCUCUAG |
| siRNA 6152 | 6152 | UAGAGCUCUGAAAUGUCU | 12506 | AAGACAUUUCAGAGCUCUA |
| | | U | | |
| siRNA 6153 | 6153 | AGAGCUCUGAAAUGUCUU | 12507 | CAAGACAUUUCAGAGCUCU |
| | | G | | |
| siRNA 6154 | 6154 | GAGCUCUGAAAUGUCUUG | 12508 | UCAAGACAUUUCAGAGCUC |
| | | A | | |
| siRNA 6155 | 6155 | AGCUCUGAAAUGUCUUGA | 12509 | AUCAAGACAUUUCAGAGCU |
| | | U | | |
| siRNA 6156 | 6156 | GCUCUGAAAUGUCUUGAU | 12510 | CAUCAAGACAUUUCAGAGC |
| | | G | | |
| siRNA 6157 | 6157 | CUCUGAAAUGUCUUGAUG | 12511 | CCAUCAAGACAUUUCAGAG |
| | | G | | |
| siRNA 6158 | 6158 | UCUGAAAUGUCUUGAUGG | 12512 | UCCAUCAAGACAUUUCAGA |
| | | A | | |
| siRNA 6159 | 6159 | CUGAAAUGUCUUGAUGGA | 12513 | UUCCAUCAAGACAUUUCAG |
| | | A | | |
| siRNA 6160 | 6160 | UGAAAUGUCUUGAUGGAA | 12514 | CUUCCAUCAAGACAUUUCA |
| | | G | | |
| siRNA 6161 | 6161 | GAAAUGUCUUGAUGGAAG | 12515 | CCUUCCAUCAAGACAUUUC |
| | | G | | |
| siRNA 6162 | 6162 | AAAUGUCUUGAUGGAAGG | 12516 | ACCUUCCAUCAAGACAUUU |
| | | U | | |
| siRNA 6163 | 6163 | AAUGUCUUGAUGGAAGGU | 12517 | UACCUUCCAUCAAGACAUU |
| | | A | | |
| siRNA 6164 | 6164 | AUGUCUUGAUGGAAGGUA | 12518 | AUACCUUCCAUCAAGACAU |
| | | U | | |
| siRNA 6165 | 6165 | UGUCUUGAUGGAAGGUAU | 12519 | AAUACCUUCCAUCAAGACA |
| | | U | | |
| siRNA 6166 | 6166 | GUCUUGAUGGAAGGUAUU | 12520 | UAAUACCUUCCAUCAAGAC |
| | | A | | |
| siRNA 6167 | 6167 | UCUUGAUGGAAGGUAUUA | 12521 | UUAAUACCUUCCAUCAAGA |
| | | A | | |
| siRNA 6168 | 6168 | CUUGAUGGAAGGUAUUAA | 12522 | UUUAAUACCUUCCAUCAAG |
| | | A | | |
| siRNA 6169 | 6169 | UUGAUGGAAGGUAUUAAA | 12523 | GUUUAAUACCUUCCAUCAA |
| | | C | | |
| siRNA 6170 | 6170 | UGAUGGAAGGUAUUAAAC | 12524 | AGUUUAAUACCUUCCAUCA |
| | | U | | |
| siRNA 6171 | 6171 | GAUGGAAGGUAUUAAACU | 12525 | UAGUUUAAUACCUUCCAUC |
| | | A | | |
| siRNA 6172 | 6172 | AUGGAAGGUAUUAAACUA | 12526 | AUAGUUUAAUACCUUCCAU |
| | | U | | |
| siRNA 6173 | 6173 | UGGAAGGUAUUAAACUAU | 12527 | AAUAGUUUAAUACCUUCCA |
| | | U | | |
| siRNA 6174 | 6174 | GGAAGGUAUUAAACUAUU | 12528 | AAAUAGUUUAAUACCUUCC |
| | | U | | |
| siRNA 6175 | 6175 | GAAGGUAUUAAACUAUUU | 12529 | CAAAUAGUUUAAUACCUUC |
| | | G | | |
| siRNA 6176 | 6176 | AAGGUAUUAAACUAUUUG | 12530 | GCAAAUAGUUUAAUACCU |
| | | C | | U |
| siRNA 6177 | 6177 | AGGUAUUAAACUAUUUGC | 12531 | GGCAAAUAGUUUAAUACC |
| | | C | | U |
| siRNA 6178 | 6178 | GGUAUUAAACUAUUUGCC | 12532 | AGGCAAAUAGUUUAAUAC |
| | | U | | C |
| siRNA 6179 | 6179 | GUAUUAAACUAUUUGCCU | 12533 | CAGGCAAAUAGUUUAAUA |
| | | G | | C |
| siRNA 6180 | 6180 | UAUUAAACUAUUUGCCUG | 12534 | ACAGGCAAAUAGUUUAAU |
| | | U | | A |
| siRNA 6181 | 6181 | AUUAAACUAUUUGCCUGU | 12535 | AACAGGCAAAUAGUUUAA |
| | | U | | U |
| siRNA 6182 | 6182 | UUAAACUAUUUGCCUGUU | 12536 | CAACAGGCAAAUAGUUUA |
| | | G | | A |
| siRNA 6183 | 6183 | UAAACUAUUUGCCUGUUG | 12537 | ACAACAGGCAAAUAGUUU |
| | | U | | A |
| siRNA 6184 | 6184 | AAACUAUUUGCCUGUUGU | 12538 | UACAACAGGCAAAUAGUU |
| | | A | | U |
| siRNA 6185 | 6185 | AACUAUUUGCCUGUUGUAC | 12539 | GUACAACAGGCAAAUAGU |
| | | | | U |
| siRNA 6186 | 6186 | ACUAUUUGCCUGUUGUACA | 12540 | UGUACAACAGGCAAAUAG |
| | | | | U |
| siRNA 6187 | 6187 | CUAUUUGCCUGUUGUACAA | 12541 | UUGUACAACAGGCAAAUA |
| | | | | G |
| siRNA 6188 | 6188 | UAUUUGCCUGUUGUACAA | 12542 | UUUGUACAACAGGCAAAU |
| | | A | | A |
| siRNA 6189 | 6189 | AUUUGCCUGUUGUACAAA | 12543 | CUUUGUACAACAGGCAAAU |
| | | G | | |
| siRNA 6190 | 6190 | UUUGCCUGUUGUACAAAG | 12544 | UCUUUGUACAACAGGCAAA |
| | | A | | |
| siRNA 6191 | 6191 | UUGCCUGUUGUACAAAGA | 12545 | UUCUUUGUACAACAGGCAA |
| | | A | | |
| siRNA 6192 | 6192 | UGCCUGUUGUACAAAGAA | 12546 | UUUCUUUGUACAACAGGCA |
| | | A | | |
| siRNA 6193 | 6193 | GCCUGUUGUACAAAGAAA | 12547 | AUUUCUUUGUACAACAGGC |
| | | U | | |
| siRNA 6194 | 6194 | CCUGUUGUACAAAGAAAU | 12548 | CAUUUCUUUGUACAACAGG |
| | | G | | |
| siRNA 6195 | 6195 | CUGUUGUACAAAGAAAUG | 12549 | ACAUUUCUUUGUACAACAG |
| | | U | | |
| siRNA 6196 | 6196 | UGUUGUACAAAGAAAUGU | 12550 | AACAUUUCUUUGUACAACA |
| | | U | | |
| siRNA 6197 | 6197 | GUUGUACAAAGAAAUGUU | 12551 | UAACAUUUCUUUGUACAAC |
| | | A | | |
| siRNA 6198 | 6198 | UUGUACAAAGAAAUGUUA | 12552 | UUAACAUUUCUUUGUACA |
| | | A | | A |
| siRNA 6199 | 6199 | UGUACAAAGAAAUGUUAA | 12553 | CUUAACAUUUCUUUGUACA |
| | | G | | |
| siRNA 6200 | 6200 | GUACAAAGAAAUGUUAAG | 12554 | UCUUAACAUUUCUUUGUAC |
| | | A | | |
| siRNA 6201 | 6201 | UACAAAGAAAUGUUAAGA | 12555 | GUCUUAACAUUUCUUUGU |
| | | C | | A |
| siRNA 6202 | 6202 | ACAAAGAAAUGUUAAGAC | 12556 | AGUCUUAACAUUUCUUUG |
| | | U | | U |
| siRNA 6203 | 6203 | CAAAGAAAUGUUAAGACU | 12557 | GAGUCUUAACAUUUCUUU |
| | | C | | G |
| siRNA 6204 | 6204 | AAAGAAAUGUUAAGACUC | 12558 | CGAGUCUUAACAUUUCUUU |
| | | G | | |
| siRNA 6205 | 6205 | AAGAAAUGUUAAGACUCG | 12559 | ACGAGUCUUAACAUUUCUU |
| | | U | | |
| siRNA 6206 | 6206 | AGAAAUGUUAAGACUCGU | 12560 | CACGAGUCUUAACAUUUCU |
| | | G | | |
| siRNA 6207 | 6207 | GAAAUGUUAAGACUCGUG | 12561 | UCACGAGUCUUAACAUUUC |
| | | A | | |
| siRNA 6208 | 6208 | AAAUGUUAAGACUCGUGA | 12562 | UUCACGAGUCUUAACAUUU |
| | | A | | |
| siRNA 6209 | 6209 | AAUGUUAAGACUCGUGAA | 12563 | UUUCACGAGUCUUAACAUU |
| | | A | | |
| siRNA 6210 | 6210 | AUGUUAAGACUCGUGAAA | 12564 | UUUUCACGAGUCUUAACAU |
| | | A | | |
| siRNA 6211 | 6211 | UGUUAAGACUCGUGAAAA | 12565 | CUUUUCACGAGUCUUAACA |
| | | G | | |
| siRNA 6212 | 6212 | GUUAAGACUCGUGAAAAG | 12566 | UCUUUUCACGAGUCUUAAC |
| | | A | | |
| siRNA 6213 | 6213 | UUAAGACUCGUGAAAAGA | 12567 | UUCUUUUCACGAGUCUUAA |
| | | A | | |
| siRNA 6214 | 6214 | UAAGACUCGUGAAAAGAA | 12568 | AUUCUUUUCACGAGUCUUA |
| | | U | | |
| siRNA 6215 | 6215 | AAGACUCGUGAAAAGAAU | 12569 | AAUUCUUUUCACGAGUCUU |
| | | U | | |
| siRNA 6216 | 6216 | AGACUCGUGAAAAGAAUU | 12570 | UAAUUCUUUUCACGAGUCU |
| | | A | | |
| siRNA 6217 | 6217 | GACUCGUGAAAAGAAUUA | 12571 | GUAAUUCUUUUCACGAGUC |
| | | C | | |
| siRNA 6218 | 6218 | ACUCGUGAAAAGAAUUAC | 12572 | AGUAAUUCUUUUCACGAG |
| | | U | | U |
| siRNA 6219 | 6219 | CUCGUGAAAAGAAUUACU | 12573 | UAGUAAUUCUUUUCACGA |
| | | A | | G |
| siRNA 6220 | 6220 | UCGUGAAAAGAAUUACUA | 12574 | AUAGUAAUUCUUUUCACG |
| | | U | | A |
| siRNA 6221 | 6221 | CGUGAAAAGAAUUACUAU | 12575 | UAUAGUAAUUCUUUUCAC |
| | | A | | G |
| siRNA 6222 | 6222 | GUGAAAAGAAUUACUAUA | 12576 | UUAUAGUAAUUCUUUUCA |
| | | A | | C |
| siRNA 6223 | 6223 | UGAAAAGAAUUACUAUAA | 12577 | CUUAUAGUAAUUCUUUUC |
| | | G | | A |
| siRNA 6224 | 6224 | GAAAAGAAUUACUAUAAG | 12578 | CCUUAUAGUAAUUCUUUUC |
| | | G | | |
| siRNA 6225 | 6225 | AAAAGAAUUACUAUAAGG | 12579 | ACCUUAUAGUAAUUCUUU |
| | | U | | U |
| siRNA 6226 | 6226 | AAAGAAUUACUAUAAGGU | 12580 | UACCUUAUAGUAAUUCUU |
| | | A | | U |
| siRNA 6227 | 6227 | AAGAAUUACUAUAAGGUA | 12581 | GUACCUUAUAGUAAUUCU |
| | | C | | U |
| siRNA 6228 | 6228 | AGAAUUACUAUAAGGUAC | 12582 | AGUACCUUAUAGUAAUUC |
| | | U | | U |
| siRNA 6229 | 6229 | GAAUUACUAUAAGGUACU | 12583 | CAGUACCUUAUAGUAAUUC |
| | | G | | |
| siRNA 6230 | 6230 | AAUUACUAUAAGGUACUG | 12584 | ACAGUACCUUAUAGUAAU |
| | | U | | U |
| siRNA 6231 | 6231 | AUUACUAUAAGGUACUGU | 12585 | CACAGUACCUUAUAGUAAU |
| | | G | | |
| siRNA 6232 | 6232 | UUACUAUAAGGUACUGUG | 12586 | UCACAGUACCUUAUAGUAA |
| | | A | | |
| siRNA 6233 | 6233 | UACUAUAAGGUACUGUGA | 12587 | UUCACAGUACCUUAUAGUA |
| | | A | | |
| siRNA 6234 | 6234 | ACUAUAAGGUACUGUGAA | 12588 | UUUCACAGUACCUUAUAGU |
| | | A | | |
| siRNA 6235 | 6235 | CUAUAAGGUACUGUGAAA | 12589 | AUUUCACAGUACCUUAUAG |
| | | U | | |
| siRNA 6236 | 6236 | UAUAAGGUACUGUGAAAU | 12590 | UAUUUCACAGUACCUUAUA |
| | | A | | |
| siRNA 6237 | 6237 | AUAAGGUACUGUGAAAUA | 12591 | UUAUUUCACAGUACCUUAU |
| | | A | | |
| siRNA 6238 | 6238 | UAAGGUACUGUGAAAUAA | 12592 | GUUAUUUCACAGUACCUUA |
| | | C | | |
| siRNA 6239 | 6239 | AAGGUACUGUGAAAUAAC | 12593 | AGUUAUUUCACAGUACCUU |
| | | U | | |
| siRNA 6240 | 6240 | AGGUACUGUGAAAUAACU | 12594 | CAGUUAUUUCACAGUACCU |
| | | G | | |
| siRNA 6241 | 6241 | GGUACUGUGAAAUAACUG | 12595 | GCAGUUAUUUCACAGUACC |
| | | C | | |
| siRNA 6242 | 6242 | GUACUGUGAAAUAACUGC | 12596 | CGCAGUUAUUUCACAGUAC |
| | | G | | |
| siRNA 6243 | 6243 | UACUGUGAAAUAACUGCG | 12597 | UCGCAGUUAUUUCACAGUA |
| | | A | | |
| siRNA 6244 | 6244 | ACUGUGAAAUAACUGCGA | 12598 | AUCGCAGUUAUUUCACAGU |
| | | U | | |
| siRNA 6245 | 6245 | CUGUGAAAUAACUGCGAU | 12599 | AAUCGCAGUUAUUUCACAG |
| | | U | | |
| siRNA 6246 | 6246 | UGUGAAAUAACUGCGAUU | 12600 | AAAUCGCAGUUAUUUCACA |
| | | U | | |
| siRNA 6247 | 6247 | GUGAAAUAACUGCGAUUU | 12601 | AAAAUCGCAGUUAUUUCAC |
| | | U | | |
| siRNA 6248 | 6248 | UGAAAUAACUGCGAUUUU | 12602 | CAAAAUCGCAGUUAUUUCA |
| | | G | | |
| siRNA 6249 | 6249 | GAAAUAACUGCGAUUUUG | 12603 | ACAAAAUCGCAGUUAUUUC |
| | | U | | |
| siRNA 6250 | 6250 | AAAUAACUGCGAUUUUGU | 12604 | CACAAAAUCGCAGUUAUUU |
| | | G | | |
| siRNA 6251 | 6251 | AAUAACUGCGAUUUUGUG | 12605 | UCACAAAAUCGCAGUUAUU |
| | | A | | |
| siRNA 6252 | 6252 | AUAACUGCGAUUUUGUGA | 12606 | CUCACAAAAUCGCAGUUAU |
| | | G | | |
| siRNA 6253 | 6253 | UAACUGCGAUUUUGUGAG | 12607 | GCUCACAAAAUCGCAGUUA |
| | | C | | |
| siRNA 6254 | 6254 | AACUGCGAUUUUGUGAGC | 12608 | UGCUCACAAAAUCGCAGUU |
| | | A | | |
| siRNA 6255 | 6255 | ACUGCGAUUUUGUGAGCA | 12609 | UUGCUCACAAAAUCGCAGU |
| | | A | | |
| siRNA 6256 | 6256 | CUGCGAUUUUGUGAGCAA | 12610 | UUUGCUCACAAAAUCGCAG |
| | | A | | |
| siRNA 6257 | 6257 | UGCGAUUUUGUGAGCAAA | 12611 | UUUUGCUCACAAAAUCGCA |
| | | A | | |
| siRNA 6258 | 6258 | GCGAUUUUGUGAGCAAAA | 12612 | GUUUUGCUCACAAAAUCGC |
| | | C | | |
| siRNA 6259 | 6259 | CGAUUUUGUGAGCAAAAC | 12613 | UGUUUUGCUCACAAAAUCG |
| | | A | | |
| siRNA 6260 | 6260 | GAUUUUGUGAGCAAAACA | 12614 | AUGUUUUGCUCACAAAAUC |
| | | U | | |
| siRNA 6261 | 6261 | AUUUUGUGAGCAAAACAU | 12615 | UAUGUUUUGCUCACAAAA |
| | | A | | U |
| siRNA 6262 | 6262 | UUUUGUGAGCAAAACAUA | 12616 | GUAUGUUUUGCUCACAAA |
| | | C | | A |
| siRNA 6263 | 6263 | UUUGUGAGCAAAACAUAC | 12617 | AGUAUGUUUUGCUCACAA |
| | | U | | A |
| siRNA 6264 | 6264 | UUGUGAGCAAAACAUACU | 12618 | AAGUAUGUUUUGCUCACA |
| | | U | | A |
| siRNA 6265 | 6265 | UGUGAGCAAAACAUACUU | 12619 | CAAGUAUGUUUUGCUCACA |
| | | G | | |
| siRNA 6266 | 6266 | GUGAGCAAAACAUACUUG | 12620 | CCAAGUAUGUUUUGCUCAC |
| | | G | | |
| siRNA 6267 | 6267 | UGAGCAAAACAUACUUGG | 12621 | UCCAAGUAUGUUUUGCUCA |
| | | A | | |
| siRNA 6268 | 6268 | GAGCAAAACAUACUUGGA | 12622 | UUCCAAGUAUGUUUUGCUC |
| | | A | | |
| siRNA 6269 | 6269 | AGCAAAACAUACUUGGAA | 12623 | UUUCCAAGUAUGUUUUGC |
| | | A | | U |
| siRNA 6270 | 6270 | GCAAAACAUACUUGGAAA | 12624 | AUUUCCAAGUAUGUUUUG |
| | | U | | C |
| siRNA 6271 | 6271 | CAAAACAUACUUGGAAAU | 12625 | CAUUUCCAAGUAUGUUUU |
| | | G | | G |
| siRNA 6272 | 6272 | AAAACAUACUUGGAAAUG | 12626 | GCAUUUCCAAGUAUGUUU |
| | | C | | U |
| siRNA 6273 | 6273 | AAACAUACUUGGAAAUGC | 12627 | AGCAUUUCCAAGUAUGUU |
| | | U | | U |
| siRNA 6274 | 6274 | AACAUACUUGGAAAUGCU | 12628 | CAGCAUUUCCAAGUAUGUU |
| | | G | | |
| siRNA 6275 | 6275 | ACAUACUUGGAAAUGCUG | 12629 | UCAGCAUUUCCAAGUAUGU |
| | | A | | |
| siRNA 6276 | 6276 | CAUACUUGGAAAUGCUGA | 12630 | AUCAGCAUUUCCAAGUAUG |
| | | U | | |
| siRNA 6277 | 6277 | AUACUUGGAAAUGCUGAU | 12631 | AAUCAGCAUUUCCAAGUAU |
| | | U | | |
| siRNA 6278 | 6278 | UACUUGGAAAUGCUGAUU | 12632 | CAAUCAGCAUUUCCAAGUA |
| | | G | | |
| siRNA 6279 | 6279 | ACUUGGAAAUGCUGAUUG | 12633 | UCAAUCAGCAUUUCCAAGU |
| | | A | | |
| siRNA 6280 | 6280 | CUUGGAAAUGCUGAUUGA | 12634 | AUCAAUCAGCAUUUCCAAG |
| | | U | | |
| siRNA 6281 | 6281 | UUGGAAAUGCUGAUUGAU | 12635 | AAUCAAUCAGCAUUUCCAA |
| | | U | | |
| siRNA 6282 | 6282 | UGGAAAUGCUGAUUGAUU | 12636 | AAAUCAAUCAGCAUUUCCA |
| | | U | | |
| siRNA 6283 | 6283 | GGAAAUGCUGAUUGAUUU | 12637 | AAAAUCAAUCAGCAUUUCC |
| | | U | | |
| siRNA 6284 | 6284 | GAAAUGCUGAUUGAUUUU | 12638 | AAAAAUCAAUCAGCAUUUC |
| | | U | | |
| siRNA 6285 | 6285 | AAAUGCUGAUUGAUUUUU | 12639 | UAAAAAUCAAUCAGCAUU |
| | | A | | U |
| siRNA 6286 | 6286 | AAUGCUGAUUGAUUUUUA | 12640 | AUAAAAAUCAAUCAGCAU |
| | | U | | U |
| siRNA 6287 | 6287 | AUGCUGAUUGAUUUUUAU | 12641 | CAUAAAAAUCAAUCAGCAU |
| | | G | | |
| siRNA 6288 | 6288 | UGCUGAUUGAUUUUUAUG | 12642 | GCAUAAAAAUCAAUCAGCA |
| | | C | | |
| siRNA 6289 | 6289 | GCUGAUUGAUUUUUAUGC | 12643 | AGCAUAAAAAUCAAUCAGC |
| | | U | | |
| siRNA 6290 | 6290 | CUGAUUGAUUUUUAUGCU | 12644 | AAGCAUAAAAAUCAAUCA |
| | | U | | G |
| siRNA 6291 | 6291 | UGAUUGAUUUUUAUGCUU | 12645 | CAAGCAUAAAAAUCAAUCA |
| | | G | | |
| siRNA 6292 | 6292 | GAUUGAUUUUUAUGCUUG | 12646 | ACAAGCAUAAAAAUCAAUC |
| | | U | | |
| siRNA 6293 | 6293 | AUUGAUUUUUAUGCUUGU | 12647 | AACAAGCAUAAAAAUCAA |
| | | U | | U |
| siRNA 6294 | 6294 | UUGAUUUUUAUGCUUGUU | 12648 | UAACAAGCAUAAAAAUCA |
| | | A | | A |
| siRNA 6295 | 6295 | UGAUUUUUAUGCUUGUUA | 12649 | CUAACAAGCAUAAAAAUCA |
| | | G | | |
| siRNA 6296 | 6296 | GAUUUUUAUGCUUGUUAG | 12650 | ACUAACAAGCAUAAAAAUC |
| | | U | | |
| siRNA 6297 | 6297 | AUUUUUAUGCUUGUUAGU | 12651 | CACUAACAAGCAUAAAAAU |
| | | G | | |
| siRNA 6298 | 6298 | UUUUUAUGCUUGUUAGUG | 12652 | ACACUAACAAGCAUAAAAA |
| | | U | | |
| siRNA 6299 | 6299 | UUUUAUGCUUGUUAGUGU | 12653 | UACACUAACAAGCAUAAAA |
| | | A | | |
| siRNA 6300 | 6300 | UUUAUGCUUGUUAGUGUA | 12654 | AUACACUAACAAGCAUAAA |
| | | U | | |
| siRNA 6301 | 6301 | UUAUGCUUGUUAGUGUAU | 12655 | AAUACACUAACAAGCAUAA |
| | | U | | |
| siRNA 6302 | 6302 | UAUGCUUGUUAGUGUAUU | 12656 | CAAUACACUAACAAGCAUA |
| | | G | | |
| siRNA 6303 | 6303 | AUGCUUGUUAGUGUAUUG | 12657 | GCAAUACACUAACAAGCAU |
| | | C | | |
| siRNA 6304 | 6304 | UGCUUGUUAGUGUAUUGC | 12658 | UGCAAUACACUAACAAGCA |
| | | A | | |
| siRNA 6305 | 6305 | GCUUGUUAGUGUAUUGCA | 12659 | UUGCAAUACACUAACAAGC |
| | | A | | |
| siRNA 6306 | 6306 | CUUGUUAGUGUAUUGCAA | 12660 | CUUGCAAUACACUAACAAG |
| | | G | | |
| siRNA 6307 | 6307 | UUGUUAGUGUAUUGCAAG | 12661 | UCUUGCAAUACACUAACAA |
| | | A | | |
| siRNA 6308 | 6308 | UGUUAGUGUAUUGCAAGA | 12662 | UUCUUGCAAUACACUAACA |
| | | A | | |
| siRNA 6309 | 6309 | GUUAGUGUAUUGCAAGAA | 12663 | UUUCUUGCAAUACACUAAC |
| | | A | | |
| siRNA 6310 | 6310 | UUAGUGUAUUGCAAGAAA | 12664 | GUUUCUUGCAAUACACUAA |
| | | C | | |
| siRNA 6311 | 6311 | UAGUGUAUUGCAAGAAAC | 12665 | UGUUUCUUGCAAUACACUA |
| | | A | | |
| siRNA 6312 | 6312 | AGUGUAUUGCAAGAAACA | 12666 | GUGUUUCUUGCAAUACACU |
| | | C | | |
| siRNA 6313 | 6313 | GUGUAUUGCAAGAAACAC | 12667 | UGUGUUUCUUGCAAUACAC |
| | | A | | |
| siRNA 6314 | 6314 | UGUAUUGCAAGAAACACA | 12668 | CUGUGUUUCUUGCAAUACA |
| | | G | | |
| siRNA 6315 | 6315 | GUAUUGCAAGAAACACAG | 12669 | UCUGUGUUUCUUGCAAUAC |
| | | A | | |
| siRNA 6316 | 6316 | UAUUGCAAGAAACACAGA | 12670 | UUCUGUGUUUCUUGCAAU |
| | | A | | A |
| siRNA 6317 | 6317 | AUUGCAAGAAACACAGAA | 12671 | UUUCUGUGUUUCUUGCAA |
| | | A | | U |
| siRNA 6318 | 6318 | UUGCAAGAAACACAGAAA | 12672 | UUUUCUGUGUUUCUUGCA |
| | | A | | A |
| siRNA 6319 | 6319 | UGCAAGAAACACAGAAAA | 12673 | AUUUUCUGUGUUUCUUGC |
| | | U | | A |
| siRNA 6320 | 6320 | GCAAGAAACACAGAAAAU | 12674 | CAUUUUCUGUGUUUCUUGC |
| | | G | | |
| siRNA 6321 | 6321 | CAAGAAACACAGAAAAUG | 12675 | ACAUUUUCUGUGUUUCUU |
| | | U | | G |
| siRNA 6322 | 6322 | AAGAAACACAGAAAAUGU | 12676 | UACAUUUUCUGUGUUUCU |
| | | A | | U |
| siRNA 6323 | 6323 | AGAAACACAGAAAAUGUA | 12677 | CUACAUUUUCUGUGUUUCU |
| | | G | | |
| siRNA 6324 | 6324 | GAAACACAGAAAAUGUAG | 12678 | ACUACAUUUUCUGUGUUUC |
| | | U | | |
| siRNA 6325 | 6325 | AAACACAGAAAAUGUAGU | 12679 | AACUACAUUUUCUGUGUU |
| | | U | | U |
| siRNA 6326 | 6326 | AACACAGAAAAUGUAGUU | 12680 | AAACUACAUUUUCUGUGU |
| | | U | | U |
| siRNA 6327 | 6327 | ACACAGAAAAUGUAGUUU | 12681 | AAAACUACAUUUUCUGUG |
| | | U | | U |
| siRNA 6328 | 6328 | CACAGAAAAUGUAGUUUU | 12682 | CAAAACUACAUUUUCUGUG |
| | | G | | |
| siRNA 6329 | 6329 | ACAGAAAAUGUAGUUUUG | 12683 | ACAAAACUACAUUUUCUGU |
| | | U | | |
| siRNA 6330 | 6330 | CAGAAAAUGUAGUUUUGU | 12684 | AACAAAACUACAUUUUCUG |
| | | U | | |
| siRNA 6331 | 6331 | AGAAAAUGUAGUUUUGUU | 12685 | AAACAAAACUACAUUUUCU |
| | | U | | |
| siRNA 6332 | 6332 | GAAAAUGUAGUUUUGUUU | 12686 | AAAACAAAACUACAUUUUC |
| | | U | | |
| siRNA 6333 | 6333 | AAAAUGUAGUUUUGUUUU | 12687 | UAAAACAAAACUACAUUU |
| | | A | | U |
| siRNA 6334 | 6334 | AAAUGUAGUUUUGUUUUA | 12688 | UUAAAACAAAACUACAUU |
| | | A | | U |
| siRNA 6335 | 6335 | AAUGUAGUUUUGUUUUAA | 12689 | AUUAAAACAAAACUACAU |
| | | U | | U |
| siRNA 6336 | 6336 | AUGUAGUUUUGUUUUAAU | 12690 | UAUUAAAACAAAACUACA |
| | | A | | U |
| siRNA 6337 | 6337 | UGUAGUUUUGUUUUAAUA | 12691 | UUAUUAAAACAAAACUAC |
| | | A | | A |
| siRNA 6338 | 6338 | GUAGUUUUGUUUUAAUAA | 12692 | UUUAUUAAAACAAAACUA |
| | | A | | C |
| siRNA 6339 | 6339 | UAGUUUUGUUUUAAUAAA | 12693 | GUUUAUUAAAACAAAACU |
| | | C | | A |
| siRNA 6340 | 6340 | AGUUUUGUUUUAAUAAAC | 12694 | GGUUUAUUAAAACAAAAC |
| | | C | | U |
| siRNA 6341 | 6341 | GUUUUGUUUUAAUAAACC | 12695 | UGGUUUAUUAAAACAAAA |
| | | A | | C |
| siRNA 6342 | 6342 | UUUUGUUUUAAUAAACCA | 12696 | UUGGUUUAUUAAAACAAA |
| | | A | | A |
| siRNA 6343 | 6343 | UUUGUUUUAAUAAACCAA | 12697 | UUUGGUUUAUUAAAACAA |
| | | A | | A |
| siRNA 6344 | 6344 | UUGUUUUAAUAAACCAAA | 12698 | UUUUGGUUUAUUAAAACA |
| | | A | | A |
| siRNA 6345 | 6345 | UGUUUUAAUAAACCAAAA | 12699 | UUUUUGGUUUAUUAAAAC |
| | | A | | A |
| siRNA 6346 | 6346 | GUUUUAAUAAACCAAAAA | 12700 | AUUUUUGGUUUAUUAAAA |
| | | U | | C |
| siRNA 6347 | 6347 | UUUUAAUAAACCAAAAAU | 12701 | AAUUUUUGGUUUAUUAAA |
| | | U | | A |
| siRNA 6348 | 6348 | UUUAAUAAACCAAAAAUU | 12702 | CAAUUUUUGGUUUAUUAA |
| | | G | | A |
| siRNA 6349 | 6349 | UUAAUAAACCAAAAAUUG | 12703 | UCAAUUUUUGGUUUAUUA |
| | | A | | A |
| siRNA 6350 | 6350 | UAAUAAACCAAAAAUUGA | 12704 | UUCAAUUUUUGGUUUAUU |
| | | A | | A |
| siRNA 6351 | 6351 | AAUAAACCAAAAAUUGAA | 12705 | GUUCAAUUUUUGGUUUAU |
| | | C | | U |
| siRNA 6352 | 6352 | AUAAACCAAAAAUUGAAC | 12706 | UGUUCAAUUUUUGGUUUA |
| | | A | | U |
| siRNA 6353 | 6353 | UAAACCAAAAAUUGAACA | 12707 | AUGUUCAAUUUUUGGUUU |
| | | U | | A |
| siRNA 6354 | 6354 | AAACCAAAAAUUGAACAU | 12708 | UAUGUUCAAUUUUUGGUU |
| | | A | | U |

TABLE 67
Additional Sequence

| SEQ | |
|---|---|
| ID | |
| NO: | Sequence (5′ to 3′) |
| 12867 | GCGCCACTGCAGCTGGCATTGGCCGGGACTGGAAGTGCGGGCTTCTGCAGCAGCCGAAGCTGGAGC |
| | TGCTAGGGCAGCAGCGGCTCCCCTGTTGTATGGACATTCTGCACCCGAAACTGATAGCTGAGTCCTG |
| | AAGTTTTATGTTATGAAACAGAAGAACTTTCATCCCAGCACATGATTTGGGAATTACACTTTGTGACA |
| | TGGATGAATCTGCACTGACCCTTGGTACAATAGATGTTTCTTATCTGCCACATTCATCAGAATACAGT |
| | GTTGGTCGATGTAAGCACACAAGTGAGGAATGGGGTGAGTGTGGCTTTAGACCCACCATCTTCAGAT |
| | CTGCAACTTTAAAATGGAAAGAAAGCCTAATGAGTCGGAAAAGGCCATTTGTTGGAAGATGTTGTTA |
| | CTCCTGCACTCCCCAGAGCTGGGACAAATTTTTCAACCCCAGTATCCCGTCTTTGGGTTTGCGGAATG |
| | TTATTTATATCAATGAAACTCACACAAGACACCGCGGATGGCTTGCAAGACGCCTTTCTTACGTTCTT |
| | TTTATTCAAGAGCGAGATGTGCATAAGGGCATGTTTGCCACCAATGTGACTGAAAATGTGCTGAACA |
| | GCAGTAGAGTACAAGAGGCAATTGCAGAAGTGGCTGCTGAATTAAACCCTGATGGTTCTGCCCAGCA |
| | GCAATCAAAAGCCGTTAACAAAGTGAAAAAGAAAGCTAAAAGGATTCTTCAAGAAATGGTTGCCAC |
| | TGTCTCACCGGCAATGATCAGACTGACTGGGTGGGTGCTGCTAAAACTGTTCAACAGCTTCTTTTGGA |
| | ACATTCAAATTCACAAAGGTCAACTTGAGATGGTTAAAGCTGCAACTGAGACGAATTTGCCGCTTCT |
| | GTTTCTACCAGTTCATAGATCCCATATTGACTATCTGCTGCTCACTTTCATTCTCTTCTGCCATAACAT |
| | CAAAGCACCATACATTGCTTCAGGCAATAATCTCAACATCCCAATCTTCAGTACCTTGATCCATAAGC |
| | TTGGGGGCTTCTTCATACGACGAAGGCTCGATGAAACACCAGATGGACGGAAAGATGTTCTCTATAG |
| | AGCTTTGCTCCATGGGCATATAGTTGAATTACTTCGACAGCAGCAATTCTTGGAGATCTTCCTGGAAG |
| | GCACACGTTCTAGGAGTGGAAAAACCTCTTGTGCTCGGGCAGGACTTTTGTCAGTTGTGGTAGATAC |
| | TCTGTCTACCAATGTCATCCCAGACATCTTGATAATACCTGTTGGAATCTCCTATGATCGCATTATCG |
| | AAGGTCACTACAATGGTGAACAACTGGGCAAACCTAAGAAGAATGAGAGCCTGTGGAGTGTAGCAA |
| | GAGGTGTTATTAGAATGTTACGAAAAAACTATGGTTGTGTCCGAGTGGATTTTGCACAGCCATTTTCC |
| | TTAAAGGAATATTTAGAAAGCCAAAGTCAGAAACCGGTGTCTGCTCTACTTTCCCTGGAGCAAGCGT |
| | TGTTACCAGCTATACTTCCTTCAAGACCCAGTGATGCTGCTGATGAAGGTAGAGACACGTCCATTAA |
| | TGAGTCCAGAAATGCAACAGATGAATCCCTACGAAGGAGGTTGATTGCAAATCTGGCTGAGCATATT |
| | CTATTCACTGCTAGCAAGTCCTGTGCCATTATGTCCACACACATTGTGGCTTGCCTGCTCCTCTACAG |
| | ACACAGGCAGGGAATTGATCTCTCCACATTGGTCGAAGACTTCTTTGTGATGAAAGAGGAAGTCCTG |
| | GCTCGTGATTTTGACCTGGGGTTCTCAGGAAATTCAGAAGATGTAGTAATGCATGCCATACAGCTGC |
| | TGGGAAATTGTGTCACAATCACCCACACTAGCAGGAACGATGAGTTTTTTATCACCCCCAGCACAAC |
| | TGTCCCATCAGTCTTCGAACTCAACTTCTACAGCAATGGGGTACTTCATGTCTTTATCATGGAGGCCA |
| | TCATAGCTTGCAGCCTTTATGCAGTTCTGAACAAGAGGGGACTGGGGGGTCCCACTAGCACCCCACC |
| | TAACCTGATCAGCCAGGAGCAGCTGGTGCGGAAGGCGGCCAGCCTGTGCTACCTTCTCTCCAATGAA |
| | GGCACCATCTCACTGCCTTGCCAGACATTTTACCAAGTCTGCCATGAAACAGTAGGAAAGTTTATCC |
| | AGTATGGCATTCTTACAGTGGCAGAGCACGATGACCAGGAAGATATCAGTCCTAGTCTTGCTGAGCA |
| | GCAGTGGGACAAGAAGCTTCCAGAACCTTTGTCTTGGAGAAGTGATGAAGAAGATGAAGACAGTGA |
| | CTTTGGGGAGGAACAGCGAGATTGCTACCTGAAGGTGAGCCAATCCAAGGAGCACCAGCAGTTTAT |
| | CACCTTCTTACAGAGACTCCTTGGGCCTTTGCTGGAGGCCTACAGCTCTGCTGCCATCTTTGTTCACA |
| | ACTTCAGTGGTCCTGTTCCAGAACCTGAGTATCTGCAAAAGTTGCACAAATACCTAATAACCAGAAC |
| | AGAAAGAAATGTTGCAGTATATGCTGAGAGTGCCACATATTGTCTTGTGAAGAATGCTGTGAAAATG |
| | TTTAAGGATATTGGGGTTTTCAAGGAGACCAAACAAAAGAGAGTGTCTGTTTTAGAACTGAGCAGCA |
| | CTTTTCTACCTCAATGCAACCGACAAAAACTTCTAGAATATATTCTGAGTTTTGTGGTGCTGTAGGTA |
| | ACGTGTGGCACTGCTGGCAAATGAAGGTCATGAGATGAGTTCCTTGTAGGTACCAGCTTCTGGCTCA |
| | AGAGTTGAAGGTGCCATCGCAGGGTCAGGCCTGCCCTGTCCCGAAGTGATCTCCTGGAAGACAAGTG |
| | CCTTCTCCCTCCATGGATCTGTGATCTTCCCAGCTCTGCATCAACACAGCAGCCTGCAGATAACACTT |
| | GGGGGGACCTCAGCCTCTATTCGCAACTCATAATCCGTAGACTACAAGATGAAATCTCAATAAATTA |
| | TTTTTGAGTTTATTAAAGATTGACATTTTAAGTACAACTTTTAAGGACTAATTACTGTGATGGACACA |
| | GAAATGTAGCTGTGTTCTGGAACTGAATCTTACATGGTATACTTAGTGCTGCTGGGTAATTTGTTGGT |
| | ATATTATCTGGTTAGTGGTTAATGCTTCCTTTAAAAATAATTGAGTCATCCATTCACTCTTTTTCAGTT |
| | TTATCTGTCAATAGTAGCTACATTTTTAATGGGAGCACCTTTTATCCCAAAGTGCTTTATAAATTGAG |
| | TGGACTGATATATATCACACCCAGGTATCACTGTGCTGTCCTTTGCTGTCAGATTTAGAAATGTTTTT |
| | AAGAGCTATGTGAAAACAGACAATATTAGTTTAGGTCGGGAACTGAGATATTGTAATCAAATAGTTA |
| | ACATCAGGAAGTTAATTTGGCTGGCAAAATTCTAGGGAAACTTGGCCAGAAAACTGGTGTTGAAGGC |
| | TTTTGCTCATATAAACAAGTGCCATTGAGTTTCAAATGACCAGCAAATATATTTAGAACCCTTCCTGT |
| | TTTATGTCTGTACCTCGTCCACCCCTCAGGTAATACCTGCCTCTCACAGGTACAGCTGTTTCTTGGAA |
| | ATCCTCCAACCAAATAGCAGTTTTCCTAACTTGATTAGCTTGAGCTGACAGACTGTTAGAATACAGTT |
| | CTCTGGCCACAGCTGATGAGGGCTTTCTGTACTGCACACAGATTGTGTACTGCACCCCAGTCCAGGT |
| | GACTGGTACCCACTCGAGTTGTGCCGTGCACAACCTGTCCAGTATATGCATGTGGTGGCCCTACTGA |
| | CTGGTAATGGTTAGAGGCATTTATGGATTTTTAGCTTTGAGGAAAAACCATGACTTTTAACAAATTTT |
| | TATGGGTTATATGCCTAAACCCTTATGCCACATAGTGGTAAATAATTATGAAAAATGGTCTGTTCATA |
| | ATTGGTAGGTGCCTTTTGTGAGCAGGGAGCATAATTATTGGTTTATTATGGTAATTATGGTGATTTTT |
| | TAAATATCATGTAATGTTAAAACGTTTTCTAACAGTTTACTGTTGCTTATCTCCAAGATATTATGGAA |
| | TTAAGAATTTTTCCAGATGAGTGTTACATAGATTCTTTGAATTTAGTATAAAAGTACTGAGAATTAAG |
| | TTTGTACTTCCATAAGCTTGGATTTTAAACACTGATAGTATCTCATGAGTAATGTGTGTTTTGGGAGA |
| | GGGAGGGATGCTGATTGATATTTCACATTGTATGAAATACCATGTTTGAAACTCATAGCAATAATGC |
| | TATGCTGTTGTGATCCCTCTCAAGTTCTGCATTTAAAATATATTTTTTCTTTATAGGAATTGATGTATA |
| | CCATGAAGTCATTGTCAGTTGTAGTAGCTCTGATGTTGAATGAGATATCATGTTTTAGCATTCCATTT |
| | TACTGACTAGGGTAGAAGAACACTTTTCTTGGCTACATTTGGAGGATACCCAGGGAGTCTTGGGTGT |
| | TCCTTATCTGGGGAAGCAAACATTTCACTAGTCTCTTTTTTTCATCCTTTAAATTGTAAATTAAGGATT |
| | ACTCAAGCTCACCATTATTCAAGATTGGGACTCGCTTCCCAGTCGACACTCTGCCCTGCCTGTCATTG |
| | CTGCAAAGAGCTGCTGCTTTGCCAACCTAAGCAAAGAAAATACGGCTTCTCTTGCATTATTTTCCCTT |
| | TTGGTTGGTTTGTTTTCTAGAAGTACGTTCAGATGCTTTGGGGAATGCAATGTATGATTTGCTAGCTC |
| | TCTCACCACTTAACTCACTGTGAGGATAAATATGCATGCTTTTTGTAATTAACTGGTGCTTTGAAAAT |
| | CTTTTTTAAGGGAGAAAAATCTCAACCAAAGTTATGCTCATCCAGACAAGCTGACCTTTGAGTTAATT |
| | TCAGCACAACTCATTCTTCAGTGCCTCATGACTGAAAACAAAAAACAAAAAAACGAAAGCATCTTCA |
| | CAATGAAGCTTCCAGATAGCACCGTTTTGCTAAAAGATACATTCTCATTGTTTTCCAACAGTGATGGC |
| | TTCCACATAAGGTTAAACAAACTAGGTGCTTGTAAATAATTTATTACAGTTTACTCTATCGCATTTCT |
| | GTAACATGAAATGCATGCCCTTCTTCAGGGGAAGACTGTGGTCAAGTTAAAAAAAAAAAACAATATT |
| | AAACAACATGAAACTGCAGTCTGTTTTTGAAAATGAGAATGTCCTAAGTGATTCAGAAGAGAGGAG |
| | GGAAGTTGTGCACTCTGAAAATGCATGAAAAACAAAGGCAAAAACTAGTGGGAAATGTGTAGAACT |
| | GTTAACTGAGATGGCTTCGAGTCTTCCTTCTGGAATCTGTTAAATTTCACAAAGTCATGAGGGTAAAT |
| | GGAGAAAATATTTCTGGGATTACAATGAATGTAAGCCCAAATTGTGGAATTGCCAGTAACCTGGATG |
| | GGGAAAAGCATTTCCCATAGCACTCCATGTAATATGAGTGCTCTGTGAGATGTTCATCAGTGTTTTAT |
| | AGAAATGGTGTTGCTGGGAAACCAAGTTTGCACCTGGAAACTTACAATGCACTTTAGCGCAGTAAGG |
| | GCTTGGCATCCGGTAGTGAAAAACTGTCTAACCCAGCATTGCCCAAACTATTTTGACACCAGGACCT |
| | TTTTCTCCTTTGGGATACTTATGAACCTCTCACTAATGTCCTGTGGAGAACATTTTGGGAAACACTAT |
| | GTTAGATAGTTCTTTAAGGAGACAAAACGGTAATGAACAGATAGCACTGGGGCAGAATATGCATGC |
| | ATTTTGTAACGTCCAGTGTGGCGTTGAATAGATGTGTATTTCCTCCCCTGCAGAAAATAAGCACAGA |
| | AAATTATAATGTAGGTGATCGGAGCTCTTTCCTTTGATAGAGAGAACAGCCCCAATGATCCTGGCTTT |
| | TTCACTGAACGTATCAGAATACATGGATGAATTGGGGTAAATAAGGTTTTAATTCAGATCTAGAAGA |
| | AAGTATTGTACGTTTGAATGCAGATTTTTATCCACAGATAGTTGTAGTGTTTAGACATGACAGGACCT |
| | ATCGTTGAGGTTTCTAAGACTTACTATGGGCTGTAAACCTGTTTTTTAAAACTATTTTAGAAACCTGA |
| | GACTTGCCGTCTGGCATTTTAGTTTAATACAAACTAATGATTGCATTTGAAAGAGATTCTTGACCTTA |
| | TTTCTAAACGTCTAGAGCTCTGAAATGTCTTGATGGAAGGTATTAAACTATTTGCCTGTTGTACAAAG |
| | AAATGTTAAGACTCGTGAAAAGAATTACTATAAGGTACTGTGAAATAACTGCGATTTTGTGAGCAAA |
| | ACATACTTGGAAATGCTGATTGATTTTTATGCTTGTTAGTGTATTGCAAGAAACACAGAAAATGTAGT |
| | TTTGTTTTAATAAACCAAAAATTGAACATA |
| 13724 | [ETL17]suauTmcaAfGfAfGfcgAmgauguasusu |
| 13725 | [ETL17]suauTmCfaAfGfAfGfcgAmgauguasusu |
| 13726 | [ETL17]suauTmcAfAfGfAfgcGmagauguasusu |
| 13727 | [ETL17]suauTmcAfAfGfAfgcgaGmauguasusu |
| 13728 | [ETL17]suauTmcAfAfGfAfgcgAmgauguasusu |
| 13729 | [ETL17]suauTmcAfAfGfAfgcgAmgauguasusu |
| 13730 | [ETL17]suauTmcAfAfGfAfgcgAmgauguasusu |
| 13731 | [ETL17]suauTmcAfAfGfAfgcgAmgauguasusu |
| 13732 | [ETL17]suauTmcAfAfGfAfgcgAmgauguasusu |
| 13733 | [ETL17]suauTmcAfAfGfAfgcgAmgauguasusu |
| 13734 | [ETL17]suauTmcAfAfGfAfgcgAmgauguasusu |
| 13735 | [ETL17]suauTmcAfAfGfAfgcgAmgauguasusu |
| 13736 | [ETL17]scauTmgcUfUfCfAfggCmaauaaasusu |
| 13737 | [ETL17]scauTmGfcUfUfCfAfggCmaauaaasusu |
| 13738 | [ETL17]scauTmGfcUfUfCfAfgGmcaauaaasusu |
| 13739 | [ETL17]scauTmGfcUfUfCfAfggcAmauaaasusu |
| 13740 | [ETL17]scauTmgCfUfUfCfaggCmaauaaasusu |
| 13741 | [ETL17]scauTmgCfUfUfCfaggCmaauaaasusu |
| 13742 | [ETL17]scauTmgCfUfUfCfaggCmaauaaasusu |
| 13743 | [ETL17]scauTmgCfUfUfCfaggCmaauaaasusu |
| 13744 | [ETL17]scauTmgCfUfUfCfaggCmaauaaasusu |
| 13745 | [ETL17]scauTmgCfUfUfCfaggCmaauaaasusu |
| 13746 | [ETL17]scauTmgCfUfUfCfaggCmaauaaasusu |
| 13747 | [ETL17]scauTmgCfUfUfCfaggCmaauaaasusu |
| 13748 | [ETL17]saauTmacUfUfCfGfacAmgcagcasusu |
| 13749 | [ETL17]saauTmAfcUfUfCfGfacAmgcagcasusu |
| 13750 | [ETL17]saauTmaCfUfUfCfgacaGmcagcasusu |
| 13751 | [ETL17]saauTmaCfUfUfCfgaCmagcagcasusu |
| 13752 | usAfscAfuCfuCfgCfuCfuUfgAfaUfasusu |
| 13753 | usAfscAfuCfuCfgCfuCfuUfgAfaUfasusu |
| 13754 | usAfscAfuCfuCfgCfuCfuUfgAfaUfasusu |
| 13755 | usAfscAfuCfuCfgCfuCfuUfgAfaUfasusu |
| 13756 | usAfscAfuCfuCfgCfuCfuUfgAfauasusu |
| 13757 | usAfscauCfuCfgCfuCfuUfgAfaUfasusu |
| 13758 | usAfscauCfucgCfuCfuUfgAfaUfasusu |
| 13759 | usAfscauCfUfcgCfuCfuUfgAfaUfasusu |
| 13760 | usAfscaUfcUfcgCfuCfuUfgAfaUfasusu |
| 13761 | usAfscaUfcUfCfgCfuCfuUfgAfaUfasusu |
| 13762 | usAfscaUfcUfCfgcuCfuUfgAfaUfasusu |
| 13763 | usAfscaUfcUfcGfcuCfuUfgAfaUfasusu |
| 13765 | usUfsuAfuUfgCfcUfgAfaGfcAfaUfgsusu |
| 13766 | usUfsuAfuUfgCfcUfgAfaGfcAfaUfgsusu |
| 13767 | usUfsuAfuUfgCfcUfgAfaGfcAfaUfgsusu |
| 13768 | usUfsuAfuUfgCfcUfgAfaGfcAfaUfgsusu |
| 13769 | usUfsuAfuUfgCfcUfgAfaGfcAfaugsusu |
| 13770 | usUfsuauUfgCfcUfgAfaGfcAfaUfgsusu |
| 13771 | usUfsuauUfGfccUfgAfaGfcAfaUfgsusu |
| 13772 | usUfsuauUfGfcCfugaaGfcAfaUfgsusu |
| 13773 | usUfsuauuGfcCfugaaGfcAfaUfgsusu |
| 13774 | usUfsuaUfuGfcCfugAfaGfcAfaUfgsusu |
| 13775 | usUfsuaUfuGfCfcugAfaGfcAfaUfgsusu |
| 13776 | usUfsuaUfuGfcCfugaaGfcAfaUfgsusu |
| 13777 | usGfscUfgCfuGfuCfgAfaGfuAfaUfususu |
| 13778 | usGfscUfgCfuGfuCfgAfaGfuAfaUfususu |
| 13779 | usGfscUfgCfuGfuCfgAfaGfuAfaUfususu |
| 13765 | usGfscUfgCfuGfuCfgAfaGfuAfaUfususu |

What is claimed is:

1. A composition comprising an oligonucleotide that targets GPAM and when administered to a subject in an effective amount decreases circulating cholesterol, apolipoprotein B, bilirubin, alanine aminotransferase, aspartate aminotransferase, or aspartate aminotransferase in a subject.

2. The composition of claim 1, wherein the cholesterol comprises total cholesterol, low density lipoprotein cholesterol, or non-high density lipoprotein cholesterol.

3. The composition of claim 1, wherein the decreased by about 10% or more, as compared to prior to administration.

4. A composition comprising an oligonucleotide that targets GPAM and when administered to a subject in an effective amount decreases a liver fibrosis score, non-alcoholic fatty liver disease (NAFLD) activity score, or liver fat percentage in a subject.

5. The composition of claim 4, wherein the decrease is by about 10% or more, as compared to prior to administration.

6. A composition comprising an oligonucleotide that targets GPAM and when administered to a subject in an effective amount decreases a use of statin (HMG CoA reductase inhibitor) medication.

7. The composition of claim 6, wherein the decrease is by about 10% or more, as compared to prior to administration.

8. A composition comprising an oligonucleotide that targets GPAM and when administered to a subject in an effective amount decreases a measurement that reflects a phenotype of esophageal varices, portal hypertension, NAFLD, NASH, alcoholic liver disease, liver fibrosis, liver cirrhosis, hepatocellular carcinoma, hyperlipidemia, ischemic heart disease, or coronary heart disease in a subject.

9. The composition of claim 8, wherein the decrease is by about 10% or more, as compared to prior to administration.

10. A composition comprising an oligonucleotide that targets GPAM and when administered to a subject in an effective amount increases circulating ketone bodies in a subject.

11. The composition of claim 10, wherein the increase is by about 10% or more, as compared to prior to administration.

12. The composition of any one of claims 1-11, wherein the oligonucleotide comprises a modified internucleoside linkage.

13. The composition of claim 12, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

14. The composition of claim 12, wherein the modified internucleoside linkage comprises one or more phosphorothioate linkages.

15. The composition of any one of claims 1-11, wherein the oligonucleotide comprises 1, 2, 3, 4, 5, 6, 7, 8, 9, 10, 11, 12, 13, 14, 15, 16, 17, 18, 19, or 20 modified internucleoside linkages.

16. The composition of any one of claims 1-11, wherein the oligonucleotide comprises a modified nucleoside.

17. The composition of claim 16, wherein the modified nucleoside comprises a locked nucleic acid (LNA), hexitol nucleic acid (HLA), cyclohexene nucleic acid (CeNA), 2′-O-(2-methoxyethyl), 2′-O-alkyl, 2-O-allyl, 2′-O-allyl, 2′-fluoro, or 2′-deoxy, or a combination thereof.

18. The composition of claim 16, wherein the modified nucleoside comprises an LNA.

19. The composition of claim 16, wherein the modified nucleoside comprises a 2′,4′ constrained ethyl nucleic acid.

20. The composition of claim 16, wherein the modified nucleoside comprises a 2′-O-methyl nucleoside, 2′-deoxyfluoro nucleoside, 2′-O-(2-methoxyethyl), 2′-O—N-methylacetamido (2′-O-NMA) nucleoside, a 2′-O-dimethylaminoethoxyethyl (2′-G-DMAEOE) nucleoside, 2′-O-aminopropyl (2′-O-AP) nucleoside, or 2′-ara-F, or a combination thereof.

21. The composition of claim 16, wherein the modified nucleoside comprises one or more 2′fluoro modified nucleosides.

22. The composition of claim 16, wherein the modified nucleoside comprises a 2′ O-alkyl modified nucleoside.

23. The composition of any one of claims 1-11, wherein the oligonucleotide comprises 1, 2, 3, 4, 5, 6, 7, 8, 9, 10, 11, 12, 13, 14, 15, 16, 17, 18, 19, 20, or 21 modified nucleosides.

24. The composition of any one of claims 1-11, wherein the oligonucleotide comprises a hydrophobic moiety attached at a 3′ or 5′ terminus of the oligonucleotide.

25. The composition of claim 24, wherein the hydrophobic moiety comprises cholesterol, myristoyl, palmitoyl, stearoyl, lithocholoyl, docosanoyl, docosahexaenoyl, myristyl, palmityl, stearyl, or α-tocopherol, or a combination thereof.

26. The composition of claim 24, wherein the hydrophobic moiety comprises stearyl, t-butylphenyl, n-butylphenyl, octylphenyl, dodecylphenyl, phenyl n-dodecyl, octadecylbenzamide, hexadecylbenzamide, or octadecylcyclohexyl.

27. The composition of claim 24, wherein the hydrophobic moiety comprises any one of the following structures: 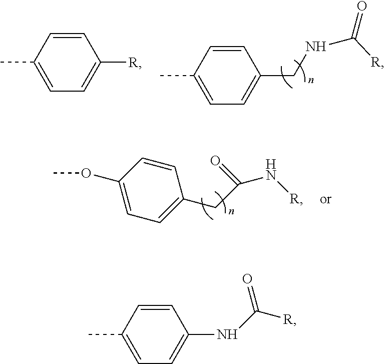
wherein the dotted line indicates a covalent connection to an end of the oligonucleotide, n is 1-3, and R is an alkyl group containing 4-18 carbons.

28. The composition of any one of claims 1-27, wherein the oligonucleotide comprises an N-acetylgalactosamine (GalNAc) moiety, an N-acetylglucosamine (GlcNAc) moiety, or a mannose moiety, attached at a 3′ or 5′ terminus of the oligonucleotide.

29. The composition of any one of claims 1-28, wherein the oligonucleotide comprises a GalNAc moiety.

30. The composition of claim 29, comprising: 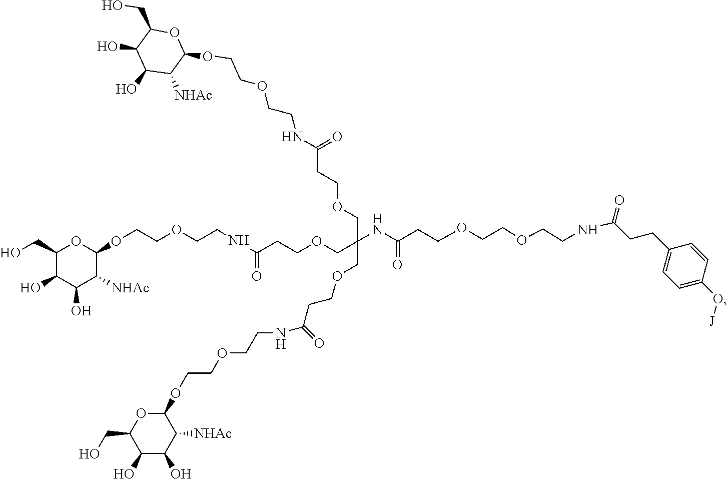
wherein J comprises the oligonucleotide, and wherein J comprises an optional phosphate or phosphorothioate linking to the oligonucleotide.

31. The composition of any one of claims 1-11, wherein the oligonucleotide comprises a small interfering RNA (siRNA) comprising a sense strand and an antisense strand.

32. The composition of claim 31, wherein the sense strand is 12-30 nucleosides in length.

33. The composition of claim 31, wherein the antisense strand is 12-30 nucleosides in length.

34. A composition comprising an oligonucleotide that inhibits the expression of GPAM, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 12867.

35. The composition of claim 31, wherein any one of the following is true with regard to the sense strand:
all purines comprise 2′ fluoro modified purines, and all pyrimidines comprise a mixture of 2′ fluoro and 2′-O-methyl modified pyrimidines;
all purines comprise 2′-O-methyl modified purines, and all pyrimidines comprise a mixture of 2′ fluoro and 2′-O-methyl modified pyrimidines;
all purines comprise 2′ fluoro modified purines, and all pyrimidines comprise 2′-O-methyl modified pyrimidines;
all pyrimidines comprise 2′ fluoro modified pyrimidines, and all purines comprise a mixture of 2′ fluoro and 2′-O-methyl modified purines;
all pyrimidines comprise 2′-O-methyl modified pyrimidines, and all purines comprise a mixture of 2′ fluoro and 2′-O-methyl modified purines; or
all pyrimidines comprise 2′ fluoro modified pyrimidines, and all purines comprise 2′-O-methyl modified purines.

36. The composition of claim 31, wherein any one of the following is true with regard to the antisense strand:
all purines comprise 2′ fluoro modified purines, and all pyrimidines comprise a mixture of 2′ fluoro and 2′-O-methyl modified pyrimidines;
all purines comprise 2′-O-methyl modified purines, and all pyrimidines comprise a mixture of 2′ fluoro and 2′-O-methyl modified pyrimidines;
all purines comprise 2′-O-methyl modified purines, and all pyrimidines comprise 2′ fluoro modified pyrimidines;
all pyrimidines comprise 2′ fluoro modified pyrimidines, and all purines comprise a mixture of 2′ fluoro and 2′-O-methyl modified purines;
all pyrimidines comprise 2′-O-methyl modified pyrimidines, and all purines comprise a mixture of 2′ fluoro and 2′-O-methyl modified purines; or
all pyrimidines comprise 2′-O-methyl modified pyrimidines, and all purines comprise 2′ fluoro modified purines.

37. The composition of any one of claims 1-11, wherein the oligonucleotide comprises an antisense oligonucleotide (ASO).

38. The composition of claim 37, wherein the ASO is 12-30 nucleosides in length.

39. A composition comprising an oligonucleotide that inhibits the expression of GPAM, wherein the oligonucleotide comprises an ASO about 12-30 nucleosides in length and a nucleoside sequence complementary to about 12-30 contiguous nucleosides of SEQ ID NO: 12867.

40. The composition of any one of claims 1-11, further comprising a pharmaceutically acceptable carrier.

41. A method of treating a subject having liver disease, comprising administering an effective amount of the composition of claim 40 to the subject.

42. The method of claim 41, wherein the liver disease comprises NAFLD, NASH, alcoholic liver disease, liver fibrosis, liver cirrhosis, or hepatocellular carcinoma.

43. A method of treating a subject having cardiometabolic disease, comprising administering an effective amount of the composition of claim 40 to the subject.

44. The method of claim 43, wherein the cardiometabolic disease comprises hyperlipidemia, ischemic heart disease, or coronary heart disease.
